SEQUENCE LISTING (4 sequences)

SEQ ID NO: 1 - protein, 188 aa, Homo sapiens [sequence omitted]

SEQ ID NO: 2 - protein, 188 aa, synthetic construct [sequence omitted]

SEQ ID NO: 3 - protein, 189 aa, Homo sapiens [sequence omitted]

SEQ ID NO: 4 - protein, 189 aa, synthetic construct
  REGION 1..189: note = KRAS4A G12D mutant
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

(19) United States
(12) Patent Application Publication
(10) Pub. No.: US 2025/0206732 A1
(43) Pub. Date: Jun. 26, 2025

(54) COMPOUNDS AND METHODS FOR THE TARGETED DEGRADATION OF KRAS

(71) Applicant: Arvinas Operations, Inc., New Haven, CT (US)

(72) Inventor: Jesus Raul MEDINA, Gilbertsville, PA (US)

(21) Appl. No.: 18/729,843

(22) PCT Filed: Jan. 20, 2023
(86) PCT No.: PCT/US23/60996

§ 371 Date: Jul. 17, 2024

Related U.S. Application Data
(60) Provisional application No. 63/301,887, filed on Jan. 21, 2022.

(51) Int. Cl. C07D 471/04; A61K 31/519; A61K 31/55; A61K 47/55; C07D 519/00

(52) U.S. Cl. CPC C07D 471/04; A61K 31/519; A61K 31/55; A61K 47/55; C07D 519/00

(57) ABSTRACT

Bifunctional compounds, which find utility as modulators of Kirsten ras sarcoma protein (KRAS), are described herein. In particular, the hetero-bifunctional compounds of the present disclosure contain on one end a moiety that binds to the Von Hippel-Lin-dau E3 ubiquitin ligase and on the other end a moiety which binds KRAS, such that the target protein is placed in proximity to the ubiquitin ligase to effect degradation (and inhibition) of target protein. The hetero-bifunctional compounds of the present disclosure exhibit a broad range of pharmacological activities associated with degradation/inhibition of target protein. Diseases or disorders that result from aberrant regulation of the target protein are treated or prevented with compounds and compositions of the present disclosure.

### RELATED APPLICATIONS

This application claims priority to U.S. Provisional Application No. 63/301,887 filed on Jan. 21, 2022, the entire content of which is hereby incorporated by reference in its entirety.

### INCORPORATION BY REFERENCE OF SEQUENCE LISTING

The instant application contains a Sequence Listing which has been submitted in ST.26 XML format via EFS-Web and is hereby incorporated by reference in its entirety. Said ST.26 XML copy, created on Jan. 20, 2023, is named “738274_ART-141PC_SeqList_ST26” and is 6 KB in size.

### BACKGROUND

Bifunctional compounds such as those described in U.S. Patent Application Publications 2015/0291562 and 2014/0356322 (incorporated herein by reference), function to recruit endogenous proteins to an E3 ubiquitin ligase for ubiquitination and subsequent degradation in the proteasome degradation pathway. In particular, the publications cited above describe bifunctional or proteolysis-targeting chimeric (PROTAC®) protein degrader compounds, which find utility as modulators of targeted ubiquitination of a variety of polypeptides and proteins, which are then degraded and/or inhibited by the bifunctional compounds.

The Kirsten rat sarcoma (KRAS) gene is an oncogene encoding KRAS, which is a small GTPase signal transduction protein. Ras proteins associate with the plasma membrane, and act as switches in the transduction of extracellular signals to intracellular response, thereby regulating, e.g., cell division. In normal cells, KRAS functions as a molecular switch, cycling between an inactive, GDP-bound “off” state and an active, GTP-bound “on” state (Milburn et al.; Ito, Y., et al., Regional polysterism in the GTP-bound form of the human c-Ha-Ras protein. Biochemistry 1997, 36 (30), 9109-9119). This switch is tightly regulated by guanine nucleotide exchange factor (GEF) proteins, which exchange GDP for GTP, and GTPase-activating proteins (GAPs), which enhance the intrinsically slow GTPase activity of KRAS (Bar-Sagi, D., The Sos (Son of sevenless) protein. Trends Endocrinol Metab 1994, 5 (4), 165-9; Pierre, S., et al., Understanding SOS (Son of Sevenless). Biochem Pharmacol 2011, 82 (9), 1049-56; Harrell Stewart, D. R., et al., Pumping the brakes on RAS-negative regulators and death effectors of RAS. J Cell Sci 2020, 133 (3)). GEF and GAP effector proteins bind at one or both of two shallow binding pockets on KRAS termed switch I (residues 30-38) and switch II (residues 59-76), the conformations of which change dramatically between GDP-bound state and GTP-bound state (Ito et al.; Boriack-Sjodin, P. A. et al., The structural basis of the activation of Ras by Sos. Nature 1998, 394 (6691), 337-43; Scheffzek, K. et al., The Ras-RasGAP complex: structural basis for GTPase activation and its loss in oncogenic Ras mutants. Science 1997, 277 (5324), 333-8).

The KRAS gene is one of the most frequently mutated oncogenes in cancer (Prior, I. A.; Lewis, P. D.; Mattos, C., A comprehensive survey of Ras mutations in cancer. Cancer Res 2012, 72 (10), 2457-67; Land, H.; Parada, L. F.; Weinberg, R. A., Tumorigenic conversion of primary embryo fibroblasts requires at least two cooperating oncogenes. Nature 1983, 304 (5927), 596-602; Newbold, R. F.; Overell, R. W., Fibroblast Immortality Is a Prerequisite for Transformation by Ej C-Ha-Ras Oncogene. Nature 1983, 304 (5927), 648-651). KRAS encodes a small, membrane bound GTPase that relays signals from receptor tyrosine kinases (RTKs), promoting cell proliferation, cell differentiation or cell death (Milburn, M. V., et al., Molecular Switch for Signal Transduction-Structural Differences between Active and Inactive Forms of Protooncogenic Ras Proteins. Science 1990, 247 (4945), 939-945; Simanshu, D. K., et al., RAS Proteins and Their Regulators in Human Disease. Cell 2017, 170 (1), 17-33). Somatic KRAS mutations attenuate the GAP-mediated enzymatic activity of the protein, resulting in accumulation of GTP-bound, active KRAS and hyperactivation of downstream signaling, which leads to uncontrolled cell proliferation (Prior et al.; Simanshu et al.). Numerous activating or gain-of-function mutations of the KRAS gene are known, and in fact, KRAS is the most frequently mutated gene in cancer. Gain-in-function KRAS mutations are found in approximately 30% of all human cancers, including, e.g., pancreatic cancer (>80%), colon cancer (approximately 40-50%), lung cancer (approximately 30-50%), non-small cell lung cancer, biliary tract malignancies, endometrial cancer, cervical cancer, bladder cancer, liver cancer, myeloid leukemia, and breast cancer. These activating mutations impair the ability of KRAS to switch between active and inactive states. Key roles for mutant KRAS have been established in initiation, maintenance, progression, and metastasis of various cancers, and mutations are frequently correlated with poor prognosis and increased resistance to chemotherapy and biological therapies, including, e.g., therapies that target epidermal growth factor receptor. However, despite its key role and prevalence in cancer, difficulties persist in developing effective therapies that directly target this oncogene. As of January 2022, Storasib (sold under the brand names Lumakras® and Lumykras®) was the only FDA-approved therapeutic directed to KRAS, and is only indicated for patients with KRAS G12C-mutated cancer, and there were no approved therapeutics targeting any other KRAS mutant. Furthermore, despite its prevalence in cancer and many years of extensive research efforts, mutant KRAS has remained a challenging therapeutic target given the scarcity of traditional druggable pockets on its surface (Spencer-Smith, R. et al., Direct inhibition of RAS: Quest for the Holy Grail? Semin Cancer Biol 2019, 54, 138-148).

An ongoing need exists in the art for effective treatments for KRAS related disease and disorders, e.g., pancreatic cancer, colon cancer, colorectal cancer, lung cancer, non-small cell lung cancer, biliary tract malignancies, endometrial cancer, cervical cancer, bladder cancer, liver cancer, myeloid leukemia, and breast cancer.

### SUMMARY

The present disclosure describes bifunctional compounds that function to recruit endogenous proteins to an E3 ubiquitin ligase for ubiquitination and degradation, and methods of using the same. In particular, the present disclosure provides bifunctional or proteolysis targeting chimeric compounds (PROTAC® protein degraders), which find utility as modulators of targeted ubiquitination of a variety of polypeptides and proteins, which are then degraded and/or otherwise inhibited by the bifunctional compounds described herein. In addition, the description provides methods of using an effective amount of the compounds described herein for the treatment or amelioration of a disease condition, such as cancer, inflammatory diseases/disorders, neurodegenerative diseases, as well as cardiovascular diseases/disorders.

In aspects, disclosed herein are bifunctional compound having the structure of Formula (Ia):

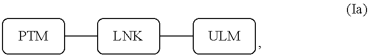

or a pharmaceutically acceptable salt, solvate, enantiomer, stereoisomer, or isotopic derivative thereof, wherein PTM is a protein/polypeptide targeting moiety, LNK is a linker, e.g. a bond (absent) or a chemical group coupling PTM to ULM, and ULM is an E3 ubiquitin ligase binding moiety. The PTM binds to a target protein or polypeptide, which is to be ubiquitinated by a ubiquitin ligase and is chemically linked directly to the ULM group or through a linker moiety LNK.

In aspects, disclosed herein are bifunctional compound having the structure of Formula (Ia):

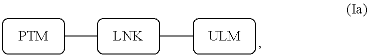

or a pharmaceutically acceptable salt thereof, wherein PTM is a protein/polypeptide targeting moiety, LNK is a linker, e.g. a bond (absent) or a chemical group coupling PTM to ULM, and ULM is an E3 ubiquitin ligase binding moiety. The PTM binds to a target protein or polypeptide, which is to be ubiquitinated by a ubiquitin ligase and is chemically linked directly to the ULM group or through a linker moiety LNK.

In aspects, disclosed herein are bifunctional compound having the structure of Formula (I):

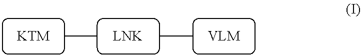

or a pharmaceutically acceptable salt, solvate, enantiomer, stereoisomer, or isotopic derivative thereof,
wherein:
KTM is a KRAS targeting moiety; LNK is a linker (e.g. a bond or a chemical linker group) covalently coupling the PTM to a Von-Hippel-Lindau (VHL) E3 ubiquitin ligase binding moiety or VLM.

In aspects, disclosed herein are bifunctional compound having the structure of Formula (I):

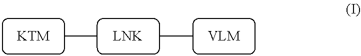

or a pharmaceutically acceptable salt thereof,
wherein:
KTM is a KRAS targeting moiety; LNK is a linker (e.g. a bond or a chemical linker group) covalently coupling the PTM to a Von-Hippel-Lindau (VHL) E3 ubiquitin ligase binding moiety or VLM.

In aspects, disclosed herein are bifunctional compound having the structure of Formula (I):

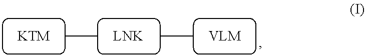

or a pharmaceutically acceptable salt, solvate, enantiomer, stereoisomer, or isotopic derivative thereof,

wherein:
(a) KTM has the structure of formula KTM-I:

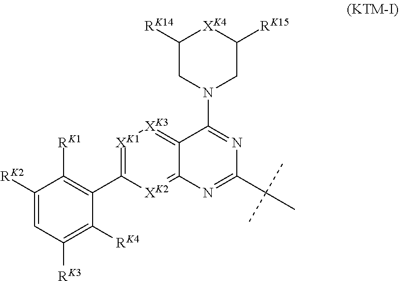

wherein:
X^{K1} is N or CR^{K5};
X^{K2} is N or CR^{K6};
X^{K3} is N or CR^{K7};
X^{K4} is NR^{K8} or C₁-C₃ alkylene, wherein the alkylene is optionally substituted with one or more R^{K9}
R^{K1} and R^{K2} are each independently selected from H, OH, Cl, F, Br, I, C₁-C₆
alkyl, C₁-C₆ haloalkyl, O—C₁-C₆ alkyl, and O—(C₁-C₆ haloalkyl);
R^{K3} and R^{K4} are each independently selected from H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), and O—(C₁-C₆ haloalkyl); or alternatively,
R^{K3} and R^{K4}, together with the carbons to which they are bonded, form C₆-C₁₀ aryl or 5- to 6-membered heteroaryl, wherein the aryl or heteroaryl is optionally substituted with one, two, three, four, or five R^{K11};
R^{K5}, R^{K6}, and R^{K7} are each independently selected from H, Cl, F, Br, I, NR^{K12}R^{K13}, C₁-C₆ alkyl, and C₁-C₆ haloalkyl;
[symbol] represents the attachment point between KTM and LNK;
R^{K8} and R^{K9} are each independently selected from H, C₁-C₆ alkyl, and C₁-C₆ haloalkyl;
each R^{K11} is independently selected from H, OH, CN, Cl, F, Br, I, NR^{K12}R^{K13} C₁-C₆ alkyl, C₂-C₆ alkenyl, C₂-C₆ alkynyl, and C₁-C₆ haloalkyl;
R^{K12} and R^{K13} are each independently selected from H, C₁-C₆ alkyl, and C₁-C₆ haloalkyl;
R^{K14} and R^{K15} are each independently selected from H, C₁-C₆ alkyl, and C₁-C₆ haloalkyl; or alternatively,
R^{K14} and R^{K15}, together with X^{K4} and the carbons to which they are bonded, form a C₄-C₇ cycloalkyl or 4- to 7-membered heterocycle;
(b) LNK is a chemical linking moiety that covalently couples the KTM to the VLM, having the structure L-I:

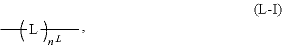

wherein:
each L is independently selected from

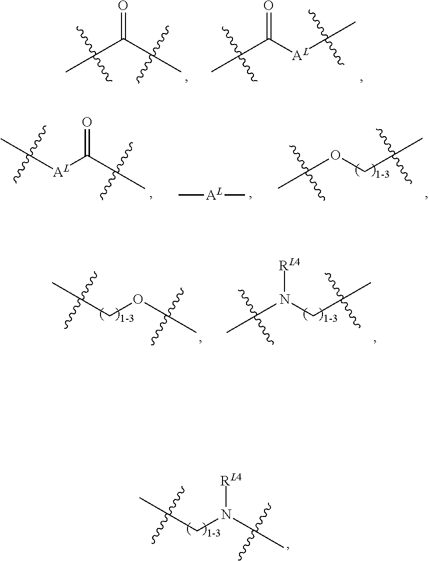

C₂-C₆ alkylene, C₂-C₆ alkenylene, C₂-C₆ alkynylene, monocyclic C₄-C₁₀ cycloalkylene, fused bicyclic C₄-C₁₀ cycloalkylene, bridged bicyclic C₆-C₁₀ cycloalkylene, or spiro-fused bicyclic C₅-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 5-12 membered heterocycloalkylene, C₆-C₁₀ arylene, and 5-6 membered heteroarylene, wherein each cycloalkylene, heterocycloalkylene, arylene, and heteroarylene is optionally substituted with one, two, three, four, or five R^{L5};
wherein each A^{L} is independently selected from CR^{L1}R^{L2}, NR^{L3}, and O;
each R^{L1} and R^{L2} is independently selected from H, C₁-C₆ alkyl, O—(C₁-C₆ alkyl), and C₁-C₆ haloalkyl, wherein the alkyl is optionally substituted with Cl, F, OH, NH₂, CN, or CF₃
each R^{L3} is independently selected from H, C₁-C₆ alkyl, O—(C₁-C₆ alkyl), and C₁-C₆ haloalkyl wherein the alkyl is optionally substituted with Cl, F, OH, NH₂, CN, or CF₃;
each R^{L4} is independently selected from C₁-C₆ alkyl, O—(C₁-C₆ alkyl), C₁-C₆ haloalkyl, NH, CN, CF₃, Cl, F, Br, I, and OH wherein the alkyl is optionally substituted with Cl, F, OH, NH₂, CN, or CF₃;
each R^{L5} is independently selected from Cl, F, Br, I, C₁-C₆ alkyl, O—(C₁-C₆ alkyl), C₁-C₆ haloalkyl, NH₂, CN, CF₃, and OH wherein the alkyl is optionally substituted with Cl, F, OH, NH₂, CN, or CF₃;
n^{L} is any integer from 1 to 50;
(c) VLM has the structure VLM-I:

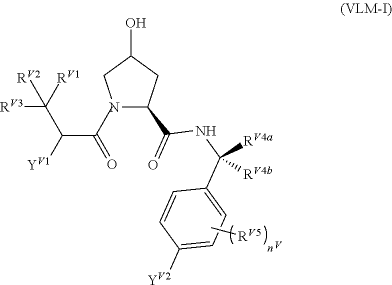

wherein:
Y^{V1} is

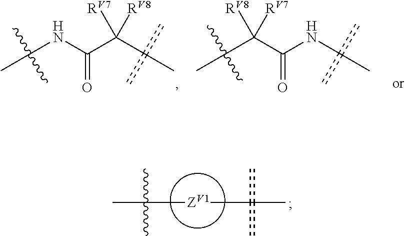

Y^{V2} is CN or

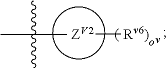

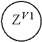

is phenylene or 5- to 6-membered heteroarylene;

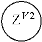

is 5-membered heteroaryl with one or two heteroatoms independently selected from N, S, and O;
R^{V1}, R^{V2}, and R^{V3} are each independently selected from H, C₁-C₆ alkyl, and C₁-C₆ haloalkyl; or, alternatively
R^{V1} and R^{V2}, together with the carbon to which they are bonded, form C₃-C₁₀ cycloalkyl or 5- to 6-membered heterocycle; and R^{V3} is selected from H, C₁-C₆ alkyl, and C₁-C₆ haloalkyl
R^{V4a} and R^{V4b} are each independently selected from H, C₁-C₆ alkyl, and C₁-C₆ haloalkyl;
each R^{V5} and R^{V6} is independently selected from H and C₁-C₆ alkyl;
R^{V7} and R^{V8} are each independently selected from H, C₁-C₆ alkyl, and C₁-C₆ haloalkyl; or, alternatively,
R^{V7} and R^{V8}, together with the atom to the carbon to which they are bonded, form C₃-C₁₀ cycloalkyl or 5- to 6-membered heterocycle;
   wherein [symbol] represents the attachment point between VLM and LNK,
n^{V} is 0, 1, 2, 3, or 4; and
o^{V} is 0, 1, 2, or 3.

In another aspect, this application pertains to a bifunctional compound having the structure of Formula (IA):

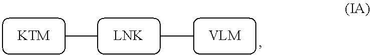

or a pharmaceutically acceptable salt, solvate, enantiomer, stereoisomer, or isotopic derivative thereof,

wherein:
(a) KTM has the structure of formula KTM-IA:

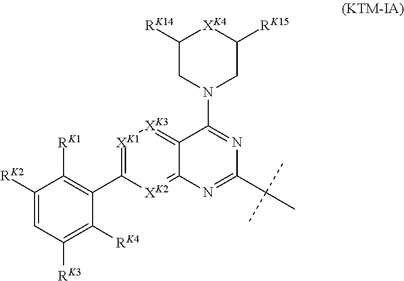

wherein:
X^{K1} is N or CR^{K5};
X^{K2} is N or CR^{K6},
X^{K3} is N or CR^{K7};
X^{K4} is NR^{K8} or C₁-C₃ alkylene, wherein the alkylene is optionally substituted with one or more R^{K9}
R^{K1} and R^{K2} are each independently selected from H, OH, Cl, F, Br, I, C₁-C₆ alkyl, C₁-C₆ haloalkyl, O—C₁-C₆ alkyl, and O—(C₁-C₆ haloalkyl);
R^{K3} and R^{K4} are each independently selected from H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), and O—(C₁-C₆ haloalkyl); or alternatively,
R^{K3} and R^{K4}, together with the carbons to which they are bonded, form C₆-C₁₀ aryl or 5- to 6-membered heteroaryl, wherein the aryl or heteroaryl is optionally substituted with one, two, three, four, or five R^{K11};
R^{K5}, R^{K6}, and R^{K7} are each independently selected from H, Cl, F, Br, I, NR^{K12}R^{K13}, C₁-C₆ alkyl, and C₁-C₆ haloalkyl;
[symbol] represents the attachment point between KTM and LNK;
R^{K8} and R^{K9} are each independently selected from H, C₁-C₆ alkyl, and C₁-C₆ haloalkyl;
each R^{K11} is independently selected from H, OH, CN, Cl, F, Br, I, NR^{K12}R^{K13} C₁-C₆ alkyl, C₂-C₆ alkenyl, C₂-C₆ alkynyl, and C₁-C₆ haloalkyl;
R^{K12} and R^{K13} are each independently selected from H, C₁-C₆ alkyl, and C₁-C₆ haloalkyl;
R^{K14} and R^{K15} are each independently selected from H, C₁-C₆ alkyl, and C₁-C₆ haloalkyl; or alternatively,
R^{K14} and R^{K15}, together with X^{K4} and the carbons to which they are bonded, form a C₄-C₇ cycloalkyl or 4- to 7-membered heterocycle;
(b) LNK is a chemical linking moiety that covalently couples the KTM to the VLM, having the structure L-IA:

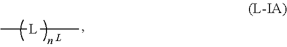

wherein:
each L is independently selected from

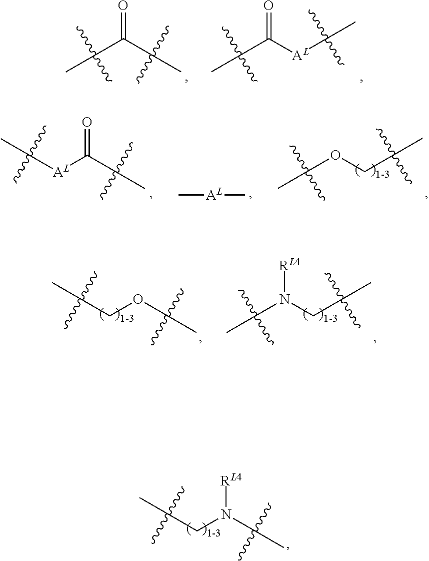

C₂-C₆ alkylene, C₂-C₆ alkenylene, C₂-C₆ alkynylene, monocyclic C₄-C₁₀ cycloalkylene, fused bicyclic C₄-C₁₀ cycloalkylene, bridged bicyclic C₆-C₁₀ cycloalkylene, or spiro-fused bicyclic C₅-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 5-12 membered heterocycloalkylene, C₆-C₁₀ arylene, and 5-6 membered heteroarylene, wherein each cycloalkylene, heterocycloalkylene, arylene, and heteroarylene is optionally substituted with one, two, three, four, or five R^{L5};
wherein each A^{L} is independently selected from CR^{L1}R^{L2}, NR^{L3}, and O;
   each R^{L1} and R^{L2} is independently selected from H, C₁-C₆ alkyl, O—(C₁-C₆ alkyl), and C₁-C₆ haloalkyl, wherein the alkyl is optionally substituted with Cl, F, OH, NH₂, CN, or CF₃
      each R^{L3} is independently selected from H, C₁-C₆ alkyl, O—(C₁-C₆ alkyl), and C₁-C₆ haloalkyl wherein the alkyl is optionally substituted with Cl, F, OH, NH₂, CN, or CF₃;
      each R^{L4} is independently selected from C₁-C₆ alkyl, O—(C₁-C₆ alkyl), C₁-C₆ haloalkyl, NH, CN, CF₃, Cl, F, Br, I, and OH wherein the alkyl is optionally substituted with Cl, F, OH, NH₂, CN, or CF₃;
      each R^{L5} is independently selected from Cl, F, Br, I, C₁-C₆ alkyl, O—(C₁-C₆ alkyl), C₁-C₆ haloalkyl, NH₂, CN, CF₃, and OH wherein the alkyl is optionally substituted with Cl, F, OH, NH₂, CN, or CF₃;
      n^{L} is any integer from 1 to 50;
(c) VLM has the structure VLM-IA:

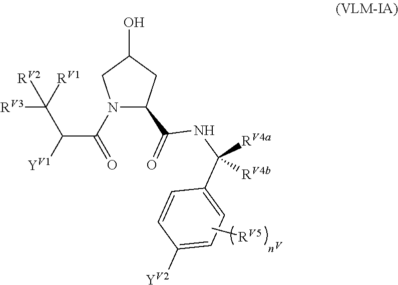

wherein:
Y^{V1} is

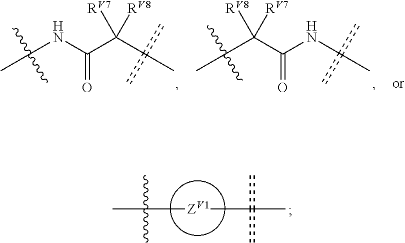

Y^{V2} is CN or

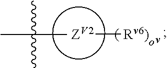

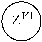

is phenylene or 5- to 6-membered heteroarylene;

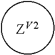

is 5-membered heteroaryl with one or two heteroatoms independently selected from N, S, and O;
R^{V1}, R^{V2}, and R^{V3} are each independently selected from H, C₁-C₆ alkyl, and C₁-C₆ haloalkyl; or, alternatively
R^{V1} and R^{V2}, together with the carbon to which they are bonded, form C₃-C₁₀ cycloalkyl or 5- to 6-membered heterocycle; and R^{V3} is selected from H, C₁-C₆ alkyl, and C₁-C₆ haloalkyl
R^{V4a} and R^{V4b} are each independently selected from H, C₁-C₆ alkyl, and C₁-C₆ haloalkyl;
each R^{V5} and R^{V6} is independently selected from H, halo, and C₁-C₆ alkyl;
R^{V7} and R^{V8} are each independently selected from H, C₁-C₆ alkyl, and C₁-C₆ haloalkyl; or, alternatively,
R^{V7} and R^{V8}, together with the atom to the carbon to which they are bonded, form C₃-C₁₀ cycloalkyl or 5- to 6-membered heterocycle;
wherein [symbol] represents the attachment point between VLM and LNK,
n^{V} is 0, 1, 2, 3, or 4; and
o^{V} is 0, 1, 2, or 3.

In embodiments, the compound of Formula IA has a structure according to Formula II:

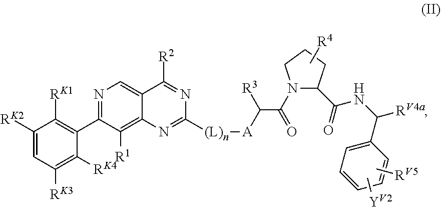

or a pharmaceutically acceptable salt thereof, wherein the variables are defined herein.

In embodiments, the compound of Formula IA has a structure according to Formula IIa:

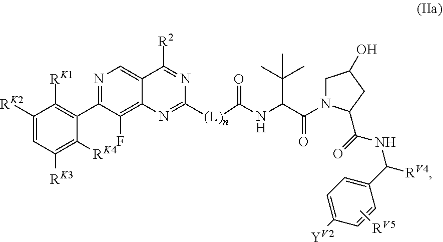

or a pharmaceutically acceptable salt thereof, wherein the variables are defined herein.

In embodiments, the compound of Formula IA has a structure according to Formula IIb:

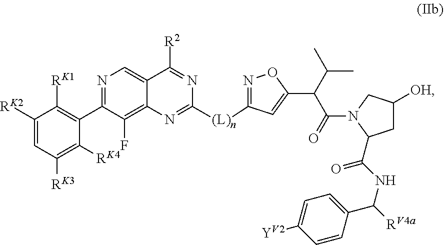

or a pharmaceutically acceptable salt thereof, wherein the variables are defined herein.

In embodiments, the compound of Formula IA has a structure according to Formula IIc:

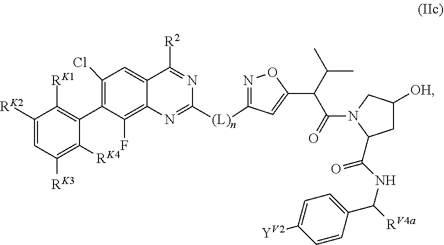

or a pharmaceutically acceptable salt thereof, wherein the variables are defined herein.

In embodiments of Formula IIa, the compound has a structure according to one of Formula IIa-i through Formula IIa-v:

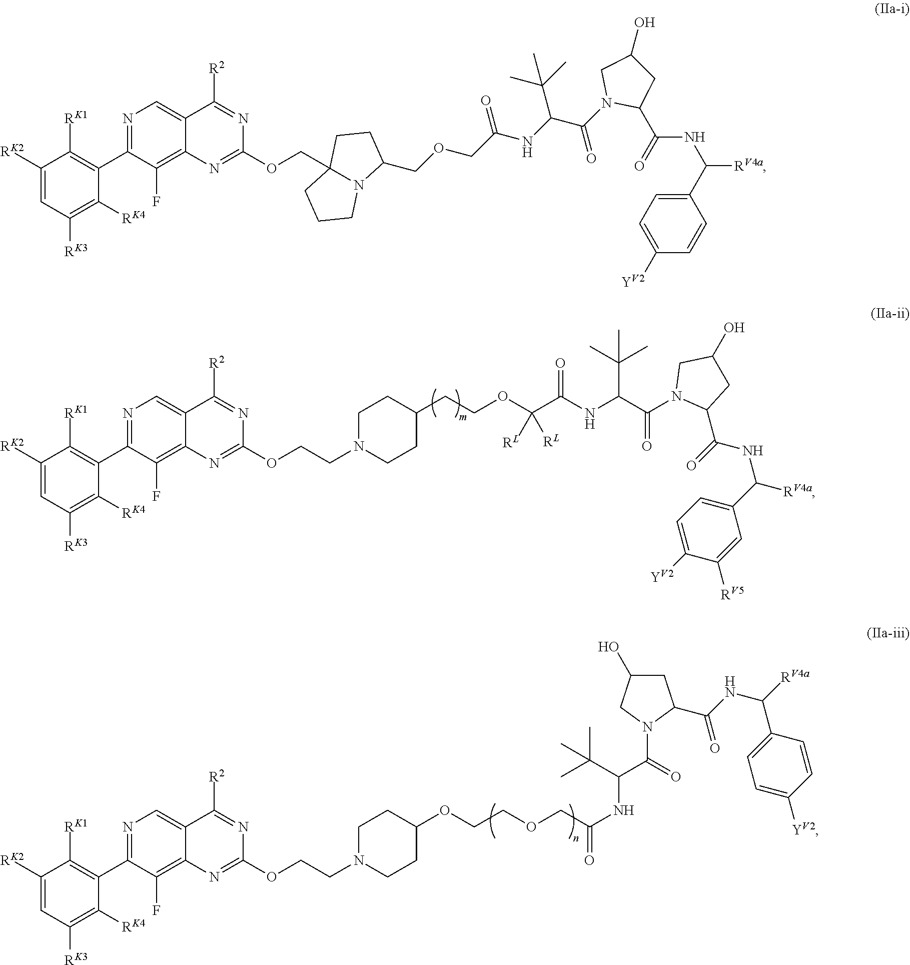

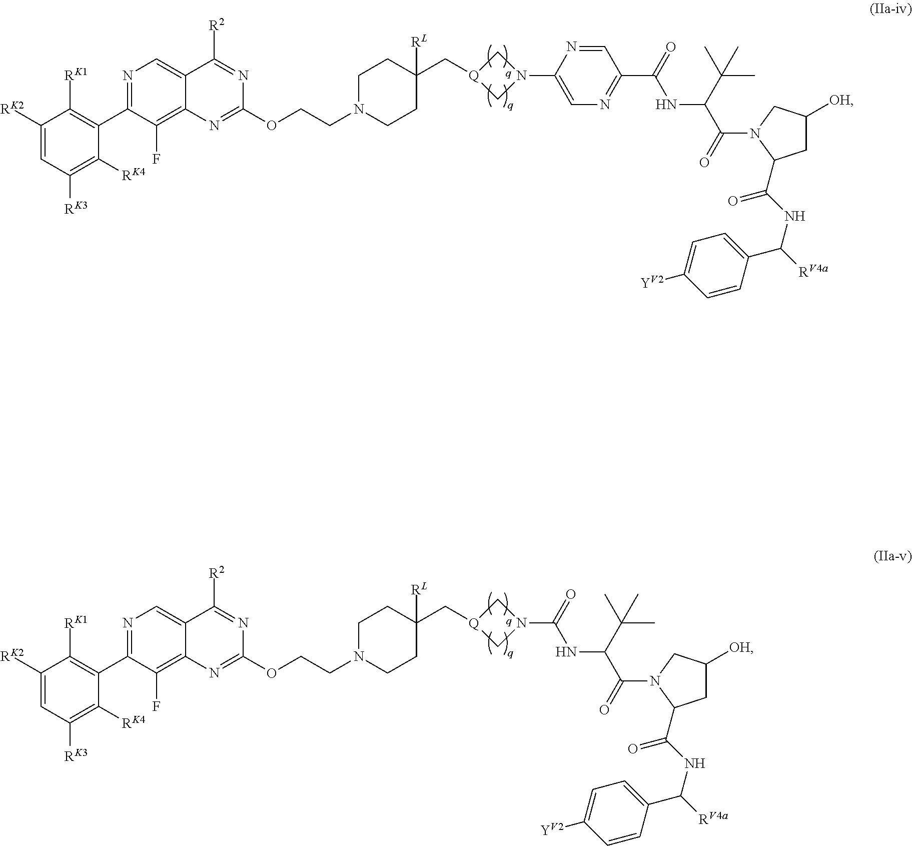

or a pharmaceutically acceptable salt thereof, wherein the variables are defined herein.

In embodiments of Formula IIb, the compound has a structure according to one of Formula IIb-i through Formula IIb-vi:

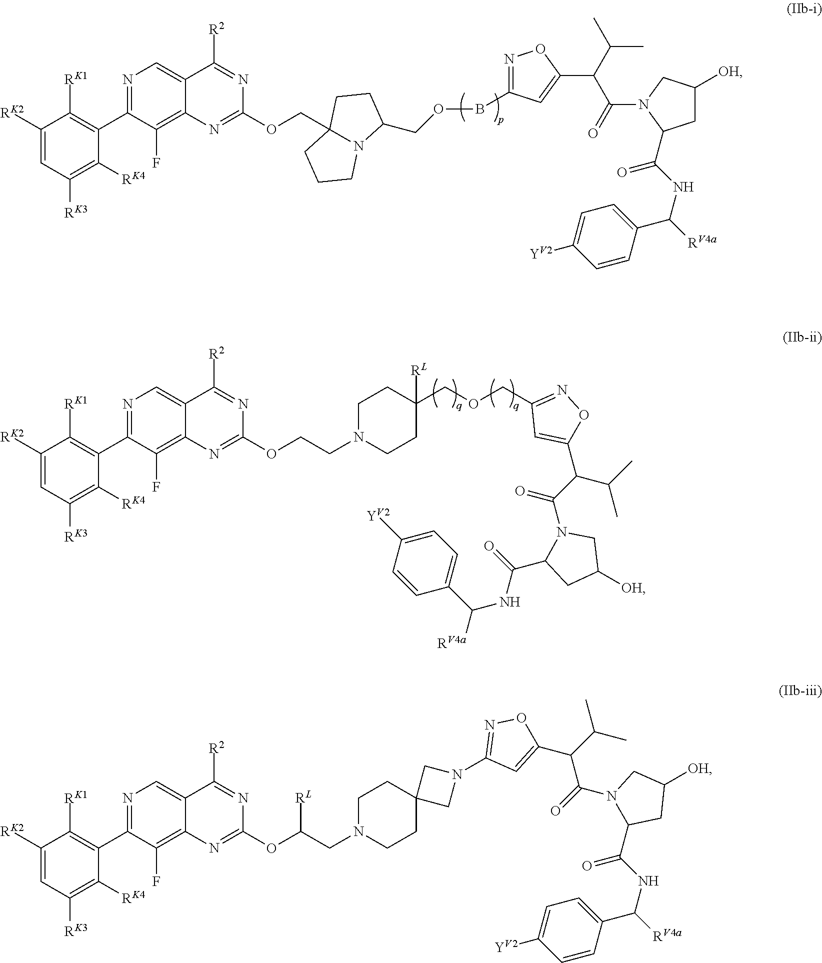

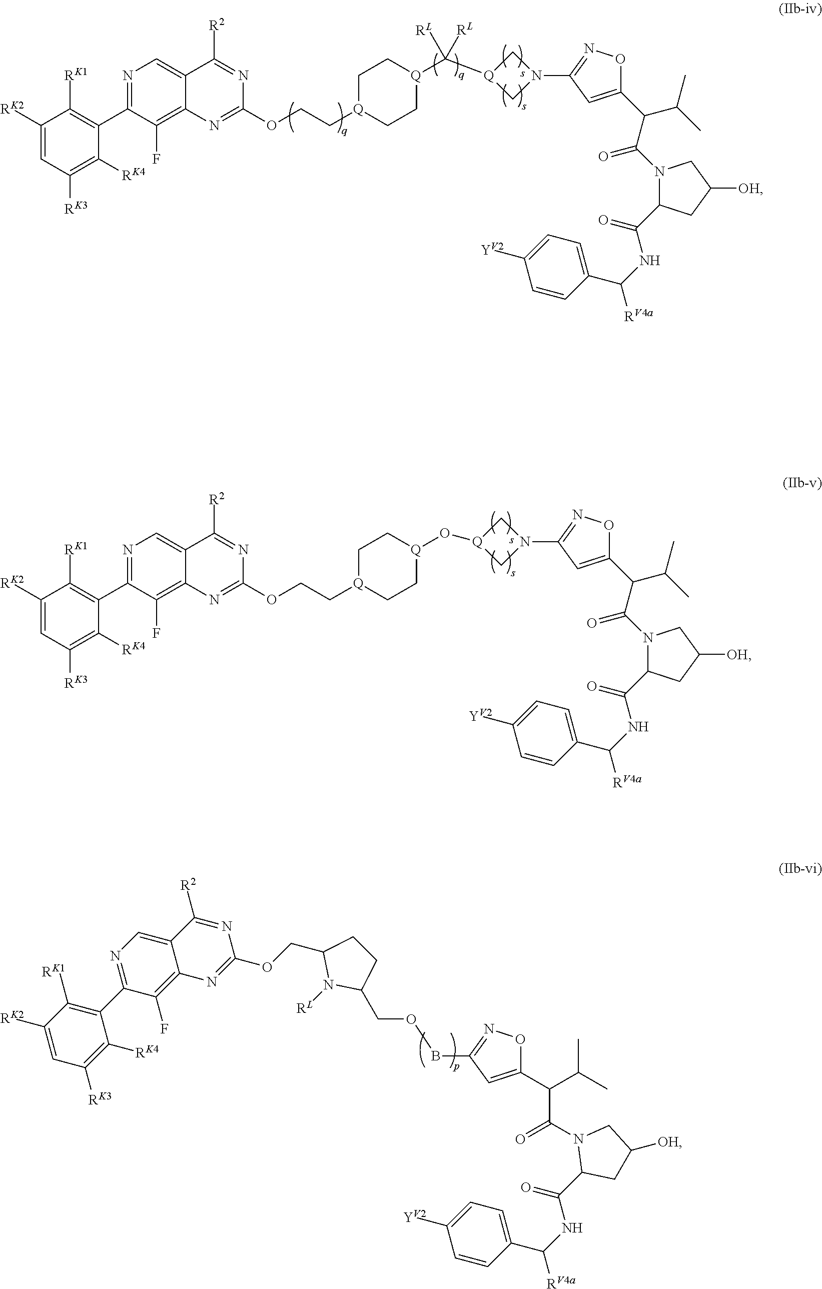

or a pharmaceutically acceptable salt thereof, wherein the variables are defined herein.

In embodiments of Formula IIc, the compound has a structure according to Formula IIc-i:

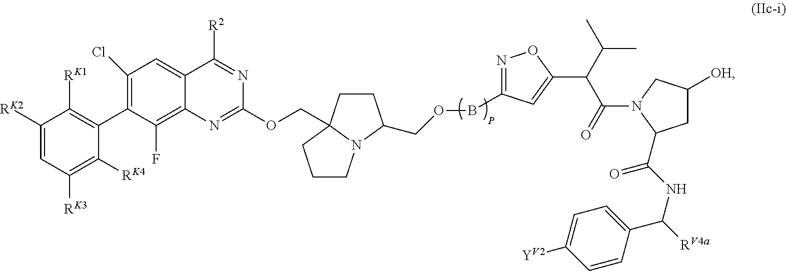

or a pharmaceutically acceptable salt thereof, wherein the variables are defined herein.

In another aspect, the present disclosure provides a pharmaceutical composition comprising a bifunctional compound of the present disclosure, or a pharmaceutically acceptable salt, solvate, enantiomer, stereoisomer, or isotopic derivative thereof, and one or more pharmaceutically acceptable excipients.

In another aspect, the present disclosure provides a method of treating a disease or disorder in a subject, the method comprising administering to the subject in need thereof a therapeutically effective amount of a bifunctional compound of the present disclosure, or a pharmaceutically acceptable salt, solvate, enantiomer, stereoisomer, or isotopic derivative thereof, or a therapeutically effective amount of a pharmaceutical composition of the present disclosure.

### BRIEF DESCRIPTION OF THE DRAWINGS

FIGS. 1A and 1B. Illustration of general principle for PROTAC function. (1A) Exemplary PROTACs comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. (1B) Illustrates the functional use of the PROTACs as described herein. Briefly, the ULM recognizes and binds to a specific E3 ubiquitin ligase, and the PTM binds and recruits a target protein bringing it into close proximity to the E3 ubiquitin ligase. Typically, the E3 ubiquitin ligase is complexed with an E2 ubiquitin conjugating protein, and either alone or via the E2 protein catalyzes attachment of ubiquitin (dark circles) to a lysine on the target protein via an isopeptide bond. The poly-ubiquitinated protein (far right) is then targeted for degradation by the proteasomal machinery of the cell.

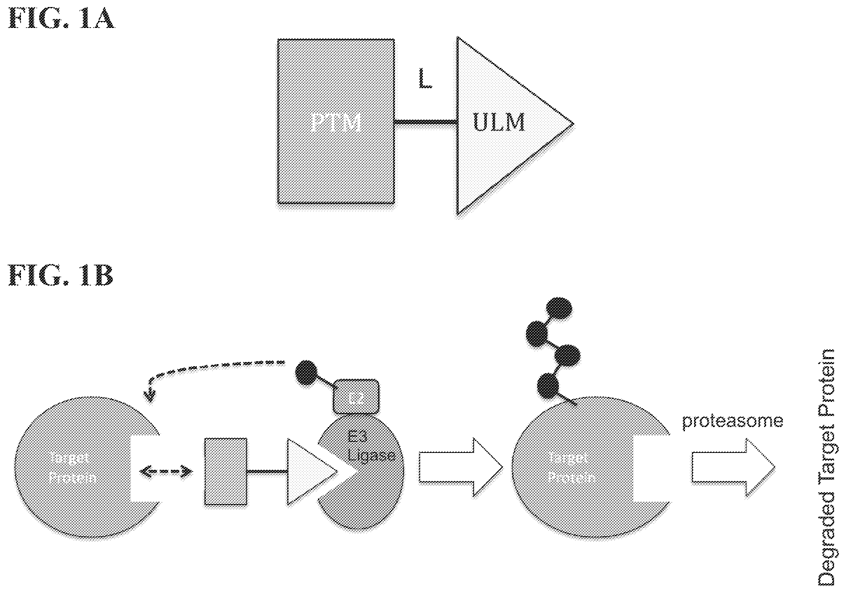

### DETAILED DESCRIPTION

In the specification, the singular forms also include the plural, unless the context clearly dictates otherwise. Unless defined otherwise, all technical and scientific terms used herein have the same meaning as commonly understood by one of ordinary skill in the art to which this invention belongs. In the case of conflict, the present specification controls. All percentages and ratios used herein, unless otherwise indicated, are by weight.

Throughout the description, where compositions are described as having, including, or comprising specific components, it is contemplated that compositions also consist essentially of, or consist of, the recited components.

Specific compounds of the present invention may be identified in the present specification by chemical name and/or chemical structure. In the event of any conflict between the chemical name and chemical structure, the chemical structure will control.

The term “alkyl”, as used herein, refers to saturated, straight-chain or branched hydrocarbon radicals containing, in certain embodiments, from one to twenty, including from one to ten, or from one to six, carbon atoms. Branched means that one or more lower C₁-C₆ alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl. Examples of C₁-C₆ alkyl radicals include, but are not limited to, methyl, ethyl, propyl, isopropyl, butyl, tert-butyl, neopentyl, n-hexyl radicals; and examples of C₁-C₈ alkyl radicals include, but are not limited to, methyl, ethyl, propyl, isopropyl, n-butyl, tert-butyl, neopentyl, n-hexyl, heptyl, octyl radicals. Examples of C₁-C₂₀ alkyl radicals include but are not limited to hexadecamethyl, hexadecaethyl, hexadecopropyl, octadecamethyl, octadecaethyl, octadecapropyl and the like. The alkyl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted.

Affixing the suffix “-ene” to a group indicates the group is a divalent moiety, e.g., alkylene (e.g., methylene (—CH₂—), ethylene (—CH₂CH₂—)) is the divalent moiety of alkyl, alkenylene is the divalent moiety of alkenyl, alkynylene is the divalent moiety of alkynyl, heteroalkylene is the divalent moiety of heteroalkyl, cycloalkylene is the divalent moiety of cycloalkyl, heterocycloalkylene is the divalent moiety of heterocycloalkyl, arylene is the divalent moiety of aryl, and heteroarylene is the divalent moiety of heteroaryl. Likewise, phenylene, oxazolylene, isoxazolylene, thiazolylene, and isothiazolylene are the divalent moieties of phenyl, oxazolyl, isoxazolyl, thiazolyl, and isothiazolyl, respectively.

The term “alkenyl”, as used herein, denotes a monovalent straight or branched group derived from a hydrocarbon moiety containing, in certain embodiments, from two to six, or two to eight, or two to twenty carbon atoms having at least one carbon-carbon double bond. The double bond may or may not be the point of attachment to another group. Examples of C₂-C₈ alkenyl groups include, but are not limited to, for example, ethenyl, propenyl, butenyl, 1-methyl-2-buten-1-yl, heptenyl, octenyl and the like. As defined herein, “alkenyl” groups include both cis- and trans-isomers. The alkenyl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆)alkyl, and —S(O)N((C₁-C₆) alkyl)₂. The substituents can themselves be optionally substituted.

The term “alkynyl”, as used herein, denotes a monovalent straight or branched group derived from a hydrocarbon moiety containing, in certain embodiments, from two to six, or two to eight, or two to twenty carbon atoms having at least one carbon-carbon triple bond. The triple bond may or may not be the point of attachment to another group. Examples of C₂-C₈ alkynyl groups include, but are not limited to, for example, ethynyl, propynyl, butynyl and the like. The alkynyl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆)alkyl, and —S(O)N((C₁-C₆) alkyl)₂. The substituents can themselves be optionally substituted.

The term “aromatic” or “aryl”, as used herein, refers to a closed ring structure which has at least one ring having a conjugated pi electron system and includes both carbocyclic aryl and heterocyclic aryl (or “heteroaryl” or “heteroaromatic”) groups. Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted. Furthermore when containing two fused rings, the aryl groups herein defined may have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

The term “C₆-C₁₀ aryl”, as used herein, refers to the cyclic, aromatic hydrocarbon groups phenyl or naphthyl, wherein said C₆-C₁₀ aryl group may be optionally substituted by one or more substituents, e.g., 1 to 5 (for phenyl) or 1 to 7 (for naphthyl) substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted. Furthermore when containing two fused rings the aryl groups herein defined may have an unsaturated or partially saturated ring fused with a fully saturated ring. Exemplary C₆-C₁₀ aryl groups include, but are not limited to, phenyl, naphthyl, and tetrahydronaphthalenyl.

One or more rings may be designated as “aromatic” by a solid circle within the ring(s). This indicates that the bonds and hydrogen atoms of the atoms in the ring are arranged so as to make the designated ring(s) aromatic. For example, the bicyclic aromatic ring naphthalene may be represented in the following interchangeable ways:

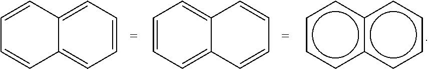

A ring may also be designated as “non-aromatic,” meaning that one of the requirements for aromaticity are not fulfilled. For example, a non-aromatic ring may contain one or more saturated carbons or may be incapable of forming a conjugated pi electron system.

Binders include, but are not limited to, hydroxypropyl methylcellulose (HPMC), hydroxypropyl cellulose (HPC), povidone, copovidone (copolymers of vinylpyrrolidone with other vinyl derivatives), methylcellulose, powdered acacia, gelatin, gum arabicum, guar gum, carbomer such as carbopol, and polymethacrylates.

Carriers include pharmaceutically acceptable excipients and diluents. The term “carrier” means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject. Examples include, but are not limited to, calcium carbonate, calcium phosphate, various sugars, starches, cellulose derivatives, gelatin, and polymers such as polyethylene glycols.

The term “cycloalkyl”, as used herein, denotes a monovalent group derived from a monocyclic or polycyclic saturated carbocyclic ring compound. Examples of C₃-C₈-cycloalkyl (3- to 8-membered cycloalkyl) include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo[2.2.1]heptyl, and bicyclo[2.2.2]octyl and the like.

When any variable (e.g., R^{K1}, R^{K2}, etc.) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present invention, one of ordinary skill in the art will recognize that the various substituents, i.e., R^{K1}, R^{K2}, etc., are to be chosen in conformity with well-known principles of chemical structure connectivity and stability. Unless expressly stated to the contrary, substitution by a named substituent is permitted on any atom in a ring (e.g., aryl, heteroaryl, cycloalkyl, heterocycloalkyl, etc.) provided such ring substitution is chemically allowed and results in a stable compound. Likewise, unless expressly stated to the contrary, when the size of a ring or chain is expressed as a range (e.g. C₁-C₆ alkyl, C₆-C₁₀ aryl, spiro-fused 5-12 membered heterocycloalkyl, etc.), the chain or ring may be selected from any size in that range, provided that such size is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

Diluents include, but are not limited to, carbohydrates such as monosaccharides like glucose, oligosaccharides like sucrose and lactose (including anhydrous lactose and lactose monohydrate), starch such as maize starch, potato starch, rice starch and wheat starch, pregelatinized starch, calcium hydrogen phosphate, and sugar alcohols like sorbitol, mannitol, erythritol, and xylitol.

Disintegrants include, but are not limited to, sodium starch glycolate, sodium carboxymethyl cellulose, calcium carboxymethyl cellulose, croscarmellose sodium, crospovidone, chitosan, agar, alginic acid, calcium alginate, methyl cellulose, microcrystalline cellulose, powdered cellulose, lower alkylsubstituted hydroxypropyl cellulose, hydroxylpropyl starch, low-substituted hydroxypropylcellulose, polacrilin potassium, starch, pregelatinized starch, sodium alginate, magnesium aluminum silicate, polacrilin potassium, povidone, sodium starch glycolate, mixtures thereof, and the like.

The term “therapeutically effective amount”, as used herein, refers to an amount of a pharmaceutical agent effective to treat, ameliorate, or prevent an identified disease, condition, or symptom, or to exhibit a detectable therapeutic or inhibitory effect. The effect can be detected by any assay or other detection method known in the art. As used herein, “therapeutically effective amount” can mean that amount necessary to make a clinically observed improvement in the patient. In some embodiments, the composition is formulated such that it comprises an amount that would not cause one or more unwanted side effects. A therapeutically effective amount of a pharmaceutical agent can also mean that amount which provides an objectively identifiable improvement as noted by a clinician or other qualified observer. The precise therapeutically effective amount for a subject will depend upon the subject's age, gender, body weight, size, and health; the nature and extent of the condition; and the therapeutic or combination of therapeutics selected for administration. Therapeutically effective amounts for a given situation can be determined by routine experimentation that is within the skill and judgment of the clinician.

Fillers include, but are not limited to, mannitol, sucrose, sorbitol, xylitol, microcrystalline cellulose, lactose, silicic acid, silicified microcrystalline cellulose, hydroxypropyl methylcellulose, hydroxypropyl cellulose, starch, pullulan and fast dissolving carbohydrates such as Pharmaburst™ fast disintegrating tablets, mixtures thereof, and the like. For examples of fast-dissolving carbohydrates see, e.g., U.S. Pat. No. 8,617,588, which is incorporated herein by reference.

Flavors include, but are not limited to, menthol, peppermint oil, peppermint spirit, vanillin, and almond oil.

Glidants include, but are not limited to, silicon dioxide, colloidal silicon dioxide, calcium silicate, magnesium silicate, magnesium trisilicate, talc, starch, mixtures thereof, and the like.

The terms “haloalkyl”, “haloalkenyl”, or “haloalkynyl”, as used herein refer to an alkyl, alkenyl or alkynyl, including straight-chain and branched, that is substituted with one or more halogens or halo groups. Examples of haloalkyl include but are not limited to CF₃, CH₂CF₃, and CCl₃.

The terms “hal”, “halo”, or “halogen”, as used herein, refer to an atom selected from fluorine, chlorine, bromine and iodine.

The term “heteroaryl”, as used herein, refers to a mono- or poly-cyclic (e.g., bi-, or tri-cyclic or more) fused or non-fused, radical or ring system having at least one aromatic ring, having from five to twelve ring atoms of which at least one ring atom is selected from S, O, P, and N. In other words, heteroaryl is aryl that contains at least one heteroatom. Examples of heteroaryl include but are not limited to pyridinyl, furanyl, thiazolyl, imidazolyl, indolyl, benzofuranyl, and the like. The heteroaryl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆)alkyl, and —S(O)N((C₁-C₆) alkyl)₂. The substituents can themselves be optionally substituted.

The term “5- or 6-membered heteroaryl”, is taken to mean a ring having five or six ring atoms of which at least one ring atom is selected from S, O, P, and N. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

“Heterocyclyl” or “heterocycloalkyl”, as used herein, are cyclic systems containing carbon and at least one heteroatom selected from N, O, S, and P, wherein there is not delocalized π electrons (aromaticity) shared among the ring carbon or heteroatoms, i.e., the cyclic ring system in non-aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl. The heterocyclyl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted.

The term “independently selected” is used herein to indicate that, for a variable which occurs in more than one location in a genus, the identity of the variable is determined separately in each instance. For example, if R^{x} appears as a substituent on two different atoms, the two instances of R^{x} may be the same moiety, or different moieties. The same is true if a single atom is substituted with more than one instance of R^{x}. The identity of R^{x} in each instance is determined independently of the identity of the other(s).

“Isomers” mean any compound having an identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.” A compound that has more than one chiral center has 2n-1 enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). The compounds of Formula (I) may contain asymmetric or chiral centers and, therefore, exist in different stereoisomeric forms. It is intended, unless specified otherwise, that all stereoisomeric forms of the compounds of Formula (I) as well as mixtures thereof, including racemic mixtures, form part of the present invention. In addition, the present invention embraces all geometric and positional isomers (including cis and trans-forms), as well as mixtures thereof, are embraced within the scope of the invention. In general, a reference to a compound is intended to cover its stereoisomers and mixture of various stereoisomers.

The present disclosure is intended to include all isotopes of atoms occurring in the present compounds. Isotopes include those atoms having the same atomic number but different mass numbers. In particular, one, some, or all hydrogens may be deuterium. Radioactive isotopes may be used, for instance for structural analysis or to facilitate tracing the fate of the compounds or their metabolic products after administration. By way of general example and without limitation, isotopes of hydrogen include deuterium and tritium and isotopes of carbon include ¹³C and ¹⁴C.

The term “isotopic derivative” includes derivatives of compounds in which one or more atoms in the compounds are replaced with corresponding isotopes of the atoms. For example, an isotopic derivative of a compound containing a carbon atom (C¹²) would be one in which one or more of the carbon atoms of the compound are replaced with the C¹³ isotope(s).

The term “KRAS” refers to polypeptide sequences forming a KRAS protein, peptide, or polypeptide (e.g. SEQ ID NO:1 and/or SEQ ID NO; 2). In some embodiments, the term “KRAS” is meant to include nucleic acid sequences encoding wild type KRAS as well KRAS protein isoforms, mutant KRAS genes, splice variants of KRAS genes, and KRAS gene polymorphisms. The term “KRAS” is used to refer to the polypeptide gene product of a KRAS gene/transcript, e.g., a KRAS protein, peptide, or polypeptide. The gene KRAS may undergo alternative splicing and thus result in two isoforms: KRAS4A (also known as KRAS2A) and KRAS4B (also known as KRAS2B). As used herein, the term “KRAS” is meant to include both isoforms.

As used herein, “KRAS G12D” refers to a mutant form of mammalian KRAS protein that contains an amino acid substitution of an aspartic acid for a glycine at amino acid position 12. As used herein, “KRAS G12V” refers to a mutant form of mammalian KRAS protein that contains an amino acid substitution of a valine for a glycine at amino acid position 12.

Lubricants include, but are not limited to, calcium stearate, glyceryl monostearate, glyceryl behenate, glyceryl palmitostearate, hexagonal boron nitride, hydrogenated vegetable oil, light mineral oil, magnesium stearate, mineral oil, polyethylene glycol, poloxamer, sodium benzoate, sodium lauryl sulfate, sodium stearyl fumarate, stearic acid, talc, zinc stearate, mixtures thereof, and the like.

“Oral dosage form” as used herein refers to a pharmaceutical drug product that contains a specified amount (dose) of a compound of the disclosure as the active ingredient, or a pharmaceutically acceptable salt and/or solvate thereof, and inactive components (excipients), formulated into a particular configuration that is suitable for oral administration, such as an oral tablet, liquid, or capsule. In some embodiments, the oral dosage form comprises a tablet. In some embodiments, the oral dosage form comprises a tablet that can be scored. In some embodiments, the oral dosage form comprises a sublingual tablet. In some embodiments, the oral dosage form comprises a capsule, which can be taken intact or used as a sprinkle onto food (e.g., applesauce or yogurt). In some embodiments, the oral dosage form comprises a sachet.

Formulations of the present invention providing “oral administration” as used herein refer to enteral, buccal, sublabial, or sublingual medications in the form of tablets, capsules, syrups, powders, granules, pastilles, solutions, tinctures, elixirs, emulsions, hydrogels, teas, films, disintegrating tablets, mouthwashes, and others.

Suitable forms for oral administration may include one or more pharmaceutically acceptable excipients, including, for example, carriers, fillers, surfactants, diluents, buffers, sweeteners, disintegrants, binders, lubricants, glidants, colorants, flavors, stabilizing agents, coatings, or any mixtures thereof.

A “pharmaceutical composition” is a formulation containing one or more therapeutic agents (e.g., one or more compounds of the present disclosure) in a form suitable for administration to a subject. In some embodiments, the pharmaceutical composition is in bulk form, e.g., for storage. Alternatively, the pharmaceutical composition is in unit dosage form. It can be advantageous to formulate compositions in unit dosage form for ease of administration and uniformity of dosage. Unit dosage form as used herein refers to physically discrete units suited as unitary dosages for the subject to be treated; each unit containing a predetermined quantity of active reagent calculated to produce the desired therapeutic effect in association with the required pharmaceutical carrier. The specifications for the unit dosage forms of the invention are dictated by and directly dependent on the unique characteristics of the active agents and the particular therapeutic effect to be achieved, and the limitations in the art of compounding such an active agent for the treatment of individuals.

A compound of the present disclosure may be administered in the form of a pharmaceutical composition comprising one or more pharmaceutically acceptable excipients. The formulation may be adapted for administration by any of a variety of routes including parenteral, buccal, rectal, vaginal, oral, intranasal, intraocular, transdermal, subcutaneous, intravenous, or intramuscular.

The term “treat,” “treated,” “treating,” or “treatment” includes the diminishment or alleviation of at least one symptom associated or caused by the state, disorder or disease being treated. In certain embodiments, the treatment comprises alleviating or preventing the symptoms of cancer.

The term “pharmaceutical” or “pharmaceutically acceptable” when used herein as an adjective, means substantially non-toxic and substantially non-deleterious to the recipient. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier or excipient” means a carrier or excipient that is useful in preparing a pharmaceutical composition that is generally safe, non-toxic and neither biologically nor otherwise undesirable, and includes any excipient that is acceptable for veterinary use and/or human pharmaceutical use. A “pharmaceutically acceptable excipient” as used herein includes both one and more than one such excipient.

As used herein, “pharmaceutically acceptable salts” can refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts can include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, or an alkaline earth metal ion, e.g., an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidinyl, pyrrolidine, benzylamine, tetramethylammonium hydroxide and the like.

It should be understood that all references to pharmaceutically acceptable salts include solvent addition forms (solvates) or crystal forms (polymorphs) as defined herein, of the same salt.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

Some of the compounds of the present disclosure may exist in unsolvated as well as solvated forms such as, for example, hydrates.

“Solvate” means a solvent addition form that contains either a stoichiometric or non-stoichiometric amounts of solvent. Some compounds can have a tendency to trap a fixed molar ratio of solvent molecules in the crystalline solid state, thus forming a solvate. If the solvent is water, the solvate formed is a hydrate; when the solvent is alcohol, the solvate formed is an alcoholate. Hydrates are formed by the combination of one or more molecules of water with one of the substances in which the water retains its molecular state as H₂O, such combination being able to form one or more hydrates. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Also suitable are the hydrates of salts of the compounds of the disclosure.

“Spirocycloalkyl” or “spirocyclyl” refers to carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₅-C₁₂) spirocycloalkyl is a spirocycle containing from 5 to 12 carbon atoms.

It will be appreciated that the compounds, as described herein, may be substituted with one, two, three, four, five or more (up to the total possible number of substituents for the particular compound) independently selected substituents or functional moieties. In general, the term “substituted” whether preceded by the term “optionally” or not, and substituents contained in formulas disclosed herein, refer to the replacement of hydrogen radicals in a given structure with the radical of a specified substituent. When more than one position in any given structure is substituted with more than one substituent selected from a specified group, the substituent may be either the same or different at each position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this disclosure, heteroatoms such as nitrogen may have hydrogen substituents and/or any permissible substituents of organic compounds described herein which satisfy the valencies of the heteroatoms. The nitrogen and sulfur heteroatoms may optionally be oxidized, and the nitrogen heteroatom may optionally be quaternized. Examples of substituents on the moieties disclosed herein (e.g., alkyl, alkenyl, alkynyl, alkoxy, aryl, heteroaryl, cycloalkyl, cycloalkenyl, non-aromatic heterocycle groups) include, but are not limited to, alkyl, alkenyl, alkynyl, halogen, haloalkyl, alkoxy, alkylthio, alkylsulfinyl, alkylsulfonyl, heteroaryl, aryl, cycloalkyl, cycloalkenyl, non-aromatic heterocycle, hydroxyl, carbamoyl, oxo, amino, nitro, azido, —SH, and —CN.

As described herein, compounds of the disclosure may optionally be substituted with one or more substituents, such as those described generally above, or as exemplified by particular classes, subclasses, and species of the disclosure. It will be appreciated that the phrase “optionally substituted” is used interchangeably with the phrase “substituted or unsubstituted.” Unless otherwise indicated, an optionally substituted group may have a substituent at any or each substitutable position of the group, and when more than one position in any given structure is substituted with more than one substituent independently selected from a specified group, the substituent may be either the same or different at each substituted every position.

Surfactants include, but are not limited to, non-ionic, anionic, cationic, amphoteric or zwitterionic surfactants. Examples of suitable non-ionic surfactants include ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™; alkyl polyglucosides; stearol ethoxylates; alkyl polyglycosides. Examples of suitable anionic surfactants include alkylether sulfates; alkylether carboxylates; alkyl benzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates. Examples of suitable cationic surfactants include fatty amine salts; fatty diamine salts; quaternary ammonium compounds; phosphonium surfactants; sulfonium surfactants; sulfoxonium surfactants. Examples of suitable zwitterionic surfactants include N-alkyl derivatives of amino acids (such as glycine, betaine, aminopropionic acid); imidazoline surfactants; amine oxides; amidobetaines. Non-limiting examples of a surfactant that can be used in solid dispersions, include, for example. Tween 20, Tween 80, Span 20, Span 80, sodium docusate (e.g., AOT), sodium lauryl sulfate, and poloxamers (e.g., poloxamer 407, Kolliphor® EL, Pluronic F68). Poloxamers are also known by the trade names Synperonics®, Pluronics®, and Kolliphor®/Cremophor®.

Sweeteners include, but are not limited to, sucrose, high fructose corn syrup, fructose, glucose, aspartame, acesulfame K, sucralose, cyclamate, sodium saccharin, neotame, rebaudioside A, and other stevia-based sweeteners.

Buffers include, but are not limited to, citrate buffer, phosphate buffer, acetate buffer and bicarbonate buffer.

### Bifunctional Compounds of Formula (Ia) and Formula (I)

In aspects, disclosed herein are bifunctional compound having the structure of Formula (Ia):

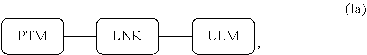

or a pharmaceutically acceptable salt, solvate, enantiomer, stereoisomer, or isotopic derivative thereof, wherein PTM is a protein/polypeptide targeting moiety, LNK is a linker, e.g. a bond (absent) or a chemical group coupling PTM to ULM, and ULM is an E3 ubiquitin ligase binding moiety. The PTM binds to a target protein or polypeptide, which is to be ubiquitinated by a ubiquitin ligase and is chemically linked directly to the ULM group or through a linker moiety LNK.

In aspects, disclosed herein are bifunctional compound having the structure of Formula (Ia):

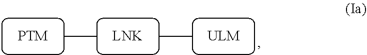

or a pharmaceutically acceptable salt thereof, wherein PTM is a protein/polypeptide targeting moiety, LNK is a linker, e.g. a bond (absent) or a chemical group coupling PTM to ULM, and ULM is an E3 ubiquitin ligase binding moiety. The PTM binds to a target protein or polypeptide, which is to be ubiquitinated by a ubiquitin ligase and is chemically linked directly to the ULM group or through a linker moiety LNK.

In aspects, disclosed herein are bifunctional compound having the structure of Formula (I):

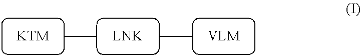

or a pharmaceutically acceptable salt, solvate, enantiomer, stereoisomer, or isotopic derivative thereof,

wherein:
KTM is a KRAS targeting moiety; LNK is a linker (e.g. a bond or a chemical linker group) covalently coupling the PTM to a Von-Hippel-Lindau (VHL) E3 ubiquitin ligase binding moiety or VLM.

In aspects, disclosed herein are bifunctional compound having the structure of Formula (I):

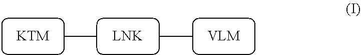

or a pharmaceutically acceptable salt thereof,

wherein:
KTM is a KRAS targeting moiety; LNK is a linker (e.g. a bond or a chemical linker group) covalently coupling the PTM to a Von-Hippel-Lindau (VHL) E3 ubiquitin ligase binding moiety or VLM.

In some embodiments, the VLM is a derivative of trans-3-hydroxyproline, where both nitrogen and carboxylic acid in trans-3-hydroxyproline are functionalized as amides. Other contemplated VLMs are described in U.S. Patent Application Publication No. 2016/0272639, U.S. Patent Application Publication No. 2014/0356322, each of which is incorporated herein by reference in its entirety.

In certain embodiments, “LNK” is a bond. In additional embodiments, the linker “LNK” is a connector with a linear non-hydrogen atom number in the range of 1 to 20. The connector “LNK” can contain, but is not limited to the functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic and tricyclic moieties. Substitution with halogen, such as Cl, F, Br and I can be included in the linker. In the case of fluorine substitution, single or multiple fluorines can be included.

In aspects, disclosed herein are bifunctional compound having the structure of Formula (I):

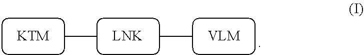

In aspects, disclosed herein are bifunctional compound having the structure of Formula (I):

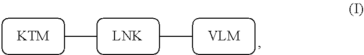

or a pharmaceutically acceptable salt, solvate, enantiomer, stereoisomer, or isotopic derivative thereof,

wherein:
(a) KTM has the structure of formula KTM-I:

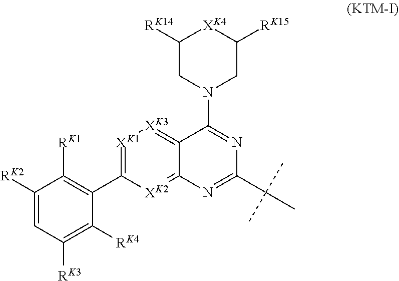

wherein:
X^{K1} is N or CR^{K5};
X^{K2} is N or CR^{K6};
X^{K3} is N or CR^{K7};
X^{K4} is NR^{K8} or C₁-C₃ alkylene, wherein the alkylene is optionally substituted with one or more R^{K9}
R^{K1} and R^{K2} are each independently selected from H, OH, Cl, F, Br, I, C₁-C₆ alkyl, C₁-C₆ haloalkyl, O—C₁-C₆ alkyl, and O—(C₁-C₆ haloalkyl);
R^{K3} and R^{K4} are each independently selected from H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), and O—(C₁-C₆ haloalkyl); or alternatively,
R^{K3} and R^{K4}, together with the carbons to which they are bonded, form C₆-C₁₀ aryl or 5- to 6-membered heteroaryl, wherein the aryl or heteroaryl is optionally substituted with one, two, three, four, or five R^{K11};
R^{K5}, R^{K6}, and R^{K7} are each independently selected from H, Cl, F, Br, I, NR^{K12}R^{K13}, C₁-C₆ alkyl, and C₁-C₆ haloalkyl;
[symbol] represents the attachment point between KTM and LNK;
R^{K8} and R^{K9} are each independently selected from H, C₁-C₆ alkyl, and C₁-C₆ haloalkyl;
each R^{K11} is independently selected from H, OH, CN, Cl, F, Br, I, NR^{K12}R^{K13} C₁-C₆ alkyl, C₂-C₆ alkenyl, C₂-C₆ alkynyl, and C₁-C₆ haloalkyl;
R^{K12} and R^{K13} are each independently selected from H, C₁-C₆ alkyl, and C₁-C₆ haloalkyl;
R^{K14} and R^{K15} are each independently selected from H, C₁-C₆ alkyl, and C₁-C₆ haloalkyl; or alternatively,
R^{K14} and R^{K15}, together with X^{K4} and the carbons to which they are bonded, form a C₄-C₇ cycloalkyl or 4- to 7-membered heterocycle;
(b) LNK is a chemical linking moiety that covalently couples the KTM to the VLM, having the structure L-I:

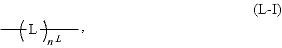

wherein:

each L is independently selected from

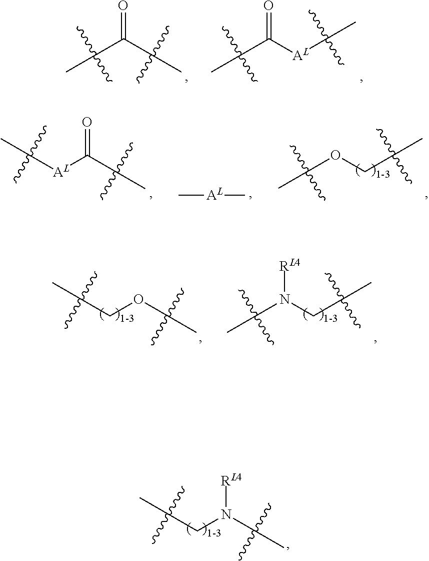

C₂-C₆ alkylene, C₂-C₆ alkenylene, C₂-C₆ alkynylene, monocyclic C₄-C₁₀ cycloalkylene, fused bicyclic C₄-C₁₀ cycloalkylene, bridged bicyclic C₆-C₁₀ cycloalkylene, or spiro-fused bicyclic C₅-C₁₂ cycloalkylene, monocyclic 4-10 membered heterocycloalkylene, bicyclic fused 4-10 membered heterocycloalkylene, bridged bicyclic 6-10 membered heterocycloalkylene, spiro-fused 5-12 membered heterocycloalkylene, C₆-C₁₀ arylene, and 5-6 membered heteroarylene, wherein each cycloalkylene, heterocycloalkylene, arylene, and heteroarylene is optionally substituted with one, two, three, four, or five R^{L5};
wherein each A^{L} is independently selected from CR^{L1}R^{L2}, NR^{L3}, and O;
   each R^{L1} and R^{L2} is independently selected from H, C₁-C₆ alkyl, O—(C₁-C₆ alkyl), and C₁-C₆ haloalkyl, wherein the alkyl is optionally substituted with Cl, F, OH, NH₂, CN, or CF₃
      each R^{L3} is independently selected from H, C₁-C₆ alkyl, O—(C₁-C₆ alkyl), and C₁-C₆ haloalkyl wherein the alkyl is optionally substituted with Cl, F, OH, NH₂, CN, or CF₃;
      each R^{L4} is independently selected from C₁-C₆ alkyl, O—(C₁-C₆ alkyl), C₁-C₆ haloalkyl, NH, CN, CF₃, Cl, F, Br, I, and OH wherein the alkyl is optionally substituted with Cl, F, OH, NH₂, CN, or CF₃;
      each R^{L5} is independently selected from Cl, F, Br, I, C₁-C₆ alkyl, O—(C₁-C₆ alkyl), C₁-C₆ haloalkyl, NH₂, CN, CF₃, and OH wherein the alkyl is optionally substituted with Cl, F, OH, NH₂, CN, or CF₃;
      n^{L} is any integer from 1 to 50;
(c) VLM has the structure VLM-I:

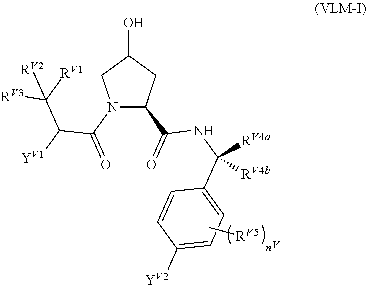

wherein:
Y^{V1} is

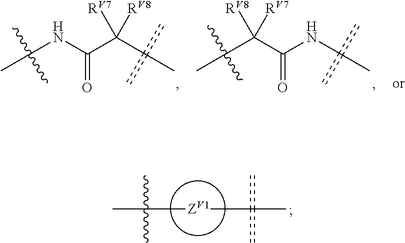

Y^{V2} is CN or

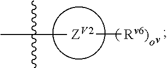

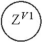

is phenylene or 5- to 6-membered heteroarylene;

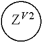

is 5-membered heteroaryl with one or two heteroatoms independently selected from N, S, and O;
R^{V1}, R^{V2}, and R^{V3} are each independently selected from H, C₁-C₆ alkyl, and C₁-C₆ haloalkyl; or, alternatively
R^{V1} and R^{V2}, together with the carbon to which they are bonded, form C₃-C₁₀ cycloalkyl or 5- to 6-membered heterocycle; and R^{V3} is selected from H, C₁-C₆ alkyl, and C₁-C₆ haloalkyl
R^{V4a} and R^{V4b} are each independently selected from H, C₁-C₆ alkyl, and C₁-C₆ haloalkyl;
each R^{V5} and R^{V6} is independently selected from H and C₁-C₆ alkyl;
R^{V7} and R^{V8} are each independently selected from H, C₁-C₆ alkyl, and C₁-C₆ haloalkyl; or, alternatively,
R^{V7} and R^{V8}, together with the atom to the carbon to which they are bonded, form C₃-C₁₀ cycloalkyl or 5- to 6-membered heterocycle;
   wherein [symbol] represents the attachment point between VLM and LNK,
n^{V} is 0, 1, 2, 3, or 4; and
o^{V} is 0, 1, 2, or 3.

In aspects, disclosed herein are bifunctional compound having the structure of Formula (I):

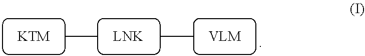

or a pharmaceutically acceptable salt, solvate, enantiomer, stereoisomer, or isotopic derivative thereof,

wherein:
(a) KTM has the structure of formula KTM-I:

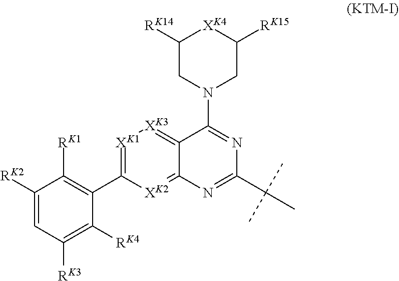

wherein:
X^{K1} is N or CR^{K5};
X^{K2} is N or CR^{K6};
X^{K3} is N or CR^{K7};
X^{K4} is NR^{K8} or C₁-C₃ alkylene, wherein the alkylene is optionally substituted with one or more R^{K9}
R^{K1} and R^{K2} are each independently selected from H, OH, Cl, F, Br, I, C₁-C₆ alkyl, C₁-C₆ haloalkyl, O—C₁-C₆ alkyl, and O—(C₁-C₆ haloalkyl);
R^{K3} and R^{K4} are each independently selected from H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), and O—(C₁-C₆ haloalkyl); or alternatively,
R^{K3} and R^{K4}, together with the carbons to which they are bonded, form C₆-C₁₀ aryl or 5- to 6-membered heteroaryl, wherein the aryl or heteroaryl is optionally substituted with one, two, three, four, or five R^{K11};
R^{K5}, R^{K6}, and R^{K7} are each independently selected from H, Cl, F, Br, I, NR^{K12}R^{K13}, C₁-C₆ alkyl, and C₁-C₆ haloalkyl;
[symbol] represents the attachment point between KTM and LNK;
R^{K8} and R^{K9} are each independently selected from H, C₁-C₆ alkyl, and C₁-C₆ haloalkyl;
each R^{K11} is independently selected from H, OH, CN, Cl, F, Br, I, NR^{K12}R^{K13}, C₁-C₆ alkyl, C₂-C₆ alkenyl, C₂-C₆ alkynyl, and C₁-C₆ haloalkyl;
R^{K12} and R^{K13} are each independently selected from H, C₁-C₆ alkyl, and C₁-C₆ haloalkyl;
R^{K14} and R^{K15} are each independently selected from H, C₁-C₆ alkyl, and C₁-C₆ haloalkyl; or alternatively,
R^{K14} and R^{K15}, together with X^{K4} and the carbons to which they are bonded, form a C₄-C₇ cycloalkyl or 4- to 7-membered heterocycle;
(b) LNK is a chemical linking moiety that covalently couples the KTM to the VLM, having the structure L-I:

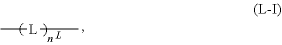

wherein:
each L is independently selected from

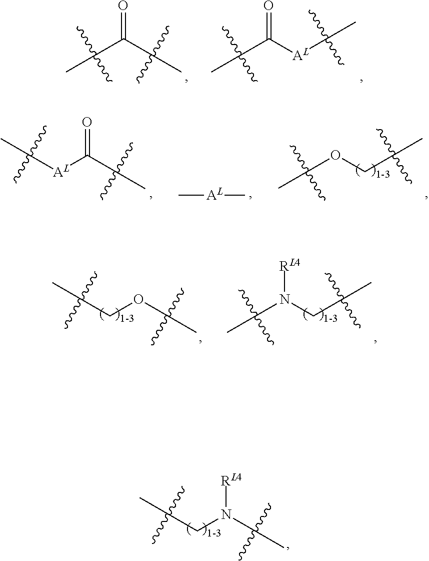

C₂-C₆ alkylene, C₂-C₆ alkenylene, C₂-C₆ alkynylene, monocyclic C₄-C₁₀ cycloalkylene, fused bicyclic C₄-C₁₀ cycloalkylene, bridged bicyclic C₆-C₁₀ cycloalkylene, or spiro-fused bicyclic C₅-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 5-12 membered heterocycloalkylene, C₆-C₁₀ arylene, and 5-6 membered heteroarylene, wherein each cycloalkylene, heterocycloalkylene, arylene, and heteroarylene is optionally substituted with one, two, three, four, or five R^{L5};
wherein each A^{L} is independently selected from CR^{L1}R^{L2}, NR^{L3}, and O;
   each R^{L1} and R^{L2} is independently selected from H, C₁-C₆ alkyl, O—(C₁-C₆ alkyl), and C₁-C₆ haloalkyl, wherein the alkyl is optionally substituted with Cl, F, OH, NH₂, CN, or CF₃
      each R^{L3} is independently selected from H, C₁-C₆ alkyl, O—(C₁-C₆ alkyl), and C₁-C₆ haloalkyl wherein the alkyl is optionally substituted with Cl, F, OH, NH₂, CN, or CF₃;
      each R^{L4} is independently selected from C₁-C₆ alkyl, O—(C₁-C₆ alkyl), C₁-C₆ haloalkyl, NH, CN, CF₃, Cl, F, Br, I, and OH wherein the alkyl is optionally substituted with Cl, F, OH, NH₂, CN, or CF₃;
      each R^{L5} is independently selected from Cl, F, Br, I, C₁-C₆ alkyl, O—(C₁-C₆ alkyl), C₁-C₆ haloalkyl, NH₂, CN, CF₃, and OH wherein the alkyl is optionally substituted with Cl, F, OH, NH₂, CN, or CF₃;
      n^{L} is 2, 3, 4, 5, or 6;
(c) VLM has the structure VLM-I:

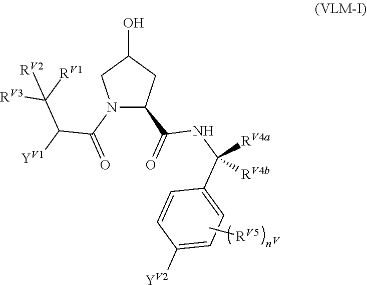

wherein:
Y^{V1} is

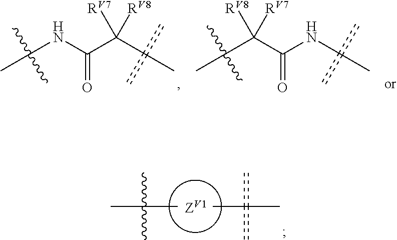

Y^{V2} is CN or

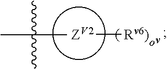

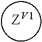

is phenylene or 5- to 6-membered heteroarylene;

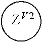

is 5-membered heteroaryl with one or two heteroatoms independently selected from N, S, and O;
R^{V1}, R^{V2}, and R^{V3} are each independently selected from H, C₁-C₆ alkyl, and C₁-C₆ haloalkyl; or, alternatively
R^{V1} and R^{V2}, together with the carbon to which they are bonded, form C₃-C₁₀ cycloalkyl or 5- to 6-membered heterocycle; and R^{V3} is selected from H, C₁-C₆ alkyl, and C₁-C₆ haloalkyl
R^{V4a} and R^{V4b} are each independently selected from H, C₁-C₆ alkyl, and C₁-C₆ haloalkyl;
each R^{V5} and R^{V6} is independently selected from H and C₁-C₆ alkyl;
R^{V7} and R^{V8} are each independently selected from H, C₁-C₆ alkyl, and C₁-C₆ haloalkyl; or, alternatively,
R^{V7} and R^{V8}, together with the atom to the carbon to which they are bonded, form C₃-C₁₀ cycloalkyl or 5- to 6-membered heterocycle;
   wherein [symbol] represents the attachment point between VLM and LNK,
n^{V} is 0, 1, 2, 3, or 4; and
o^{V} is 0, 1, 2, or 3.

In another aspect, this application pertains to a bifunctional compound having the structure of Formula (IA):

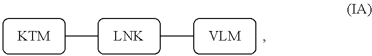

or a pharmaceutically acceptable salt, solvate, enantiomer, stereoisomer, or isotopic derivative thereof,

wherein:
(a) KTM has the structure of formula KTM-IA:

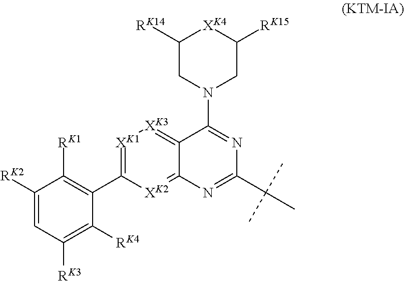

wherein:
X^{K1} is N or CR^{K5};
X^{K2} is N or CR^{K6};
X^{K3} is N or CR^{K7};
X^{K4} is NR^{K8} or C₁-C₃ alkylene, wherein the alkylene is optionally substituted with one or more R^{K9}
R^{K1} and R^{K2} are each independently selected from H, OH, Cl, F, Br, I, C₁-C₆ alkyl, C₁-C₆ haloalkyl, O—C₁-C₆ alkyl, and O—(C₁-C₆ haloalkyl);
R^{K3} and R^{K4} are each independently selected from H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), and O—(C₁-C₆ haloalkyl); or alternatively,
R^{K3} and R^{K4}, together with the carbons to which they are bonded, form C₆-C₁₀ aryl or 5- to 6-membered heteroaryl, wherein the aryl or heteroaryl is optionally substituted with one, two, three, four, or five R^{K11};
R^{K5}, R^{K6}, and R^{K7} are each independently selected from H, Cl, F, Br, I, NR^{K12}R^{K13}, C₁-C₆ alkyl, and C₁-C₆ haloalkyl;
[symbol] represents the attachment point between KTM and LNK;
R^{K8} and R^{K9} are each independently selected from H, C₁-C₆ alkyl, and C₁-C₆ haloalkyl;
each R^{K11} is independently selected from H, OH, CN, Cl, F, Br, I, NR^{K12}R^{K13} C₁-C₆ alkyl, C₂-C₆ alkenyl, C₂-C₆ alkynyl, and C₁-C₆ haloalkyl;
R^{K12} and R^{K13} are each independently selected from H, C₁-C₆ alkyl, and C₁-C₆ haloalkyl;
R^{K14} and R^{K15} are each independently selected from H, C₁-C₆ alkyl, and C₁-C₆ haloalkyl; or alternatively,
R^{K14} and R^{K15}, together with X^{K4} and the carbons to which they are bonded, form a C₄-C₇ cycloalkyl or 4- to 7-membered heterocycle;
(b) LNK is a chemical linking moiety that covalently couples the KTM to the VLM, having the structure L-IA:

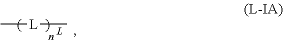

wherein:
each L is independently selected from

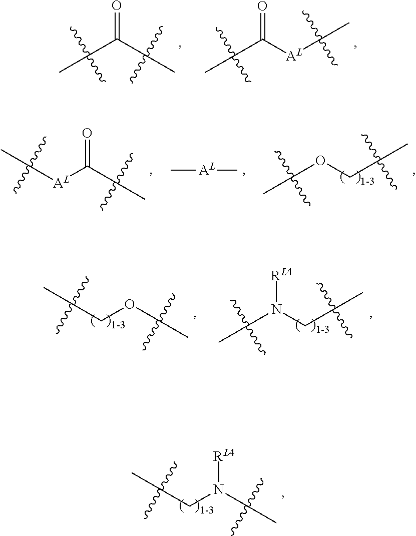

C₂-C₆ alkylene, C₂-C₆ alkenylene, C₂-C₆ alkynylene, monocyclic C₄-C₁₀ cycloalkylene, fused bicyclic C₄-C₁₀ cycloalkylene, bridged bicyclic C₆-C₁₀ cycloalkylene, or spiro-fused bicyclic C₅-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 5-12 membered heterocycloalkylene, C₆-C₁₀ arylene, and 5-6 membered heteroarylene, wherein each cycloalkylene, heterocycloalkylene, arylene, and heteroarylene is optionally substituted with one, two, three, four, or five R^{L5};
wherein each A^{L} is independently selected from CR^{L1}R^{L2}, NR^{L3}, and O;
   each R^{L1} and R^{L2} is independently selected from H, C₁-C₆ alkyl, O—(C₁-C₆ alkyl), and C₁-C₆ haloalkyl, wherein the alkyl is optionally substituted with Cl, F, OH, NH₂, CN, or CF₃
      each R^{L3} is independently selected from H, C₁-C₆ alkyl, O—(C₁-C₆ alkyl), and C₁-C₆ haloalkyl wherein the alkyl is optionally substituted with Cl, F, OH, NH₂, CN, or CF₃;
      each R^{L4} is independently selected from C₁-C₆ alkyl, O—(C₁-C₆ alkyl), C₁-C₆ haloalkyl, NH, CN, CF₃, Cl, F, Br, I, and OH wherein the alkyl is optionally substituted with Cl, F, OH, NH₂, CN, or CF₃;
      each R^{L5} is independently selected from Cl, F, Br, I, C₁-C₆ alkyl, O—(C₁-C₆ alkyl), C₁-C₆ haloalkyl, NH₂, CN, CF₃, and OH wherein the alkyl is optionally substituted with Cl, F, OH, NH₂, CN, or CF₃;
      n^{L} is any integer from 1 to 50;
(c) VLM has the structure VLM-IA:

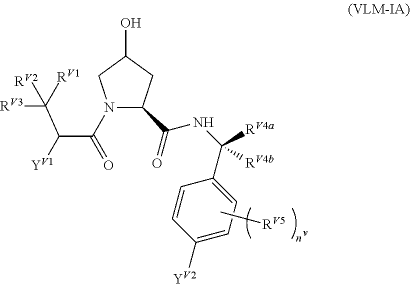

wherein:
Y^{V1} is

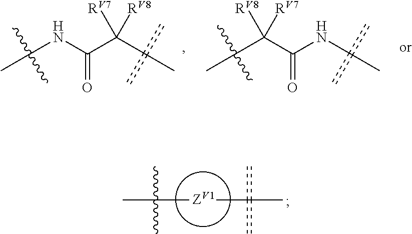

Y^{V2} is CN or

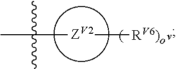

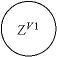

is phenylene or 5- to 6-membered heteroarylene;

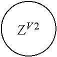

is 5-membered heteroaryl with one or two heteroatoms independently selected from N, S, and O;
R^{V1}, R^{V2}, and R^{V3} are each independently selected from H, C₁-C₆ alkyl, and C₁-C₆ haloalkyl; or, alternatively
R^{V1} and R^{V2}, together with the carbon to which they are bonded, form C₃-C₁₀ cycloalkyl or 5- to 6-membered heterocycle; and R^{V3} is selected from H, C₁-C₆ alkyl, and C₁-C₆ haloalkyl
R^{V4a} and R^{V4b} are each independently selected from H, C₁-C₆ alkyl, and C₁-C₆ haloalkyl;
each R^{V5} and R^{V6} is independently selected from H, halo, and C₁-C₆ alkyl;
R^{V7} and R^{V8} are each independently selected from H, C₁-C₆ alkyl, and C₁-C₆ haloalkyl; or, alternatively,
R^{V7} and R^{V8}, together with the atom to the carbon to which they are bonded, form C₃-C₁₀ cycloalkyl or 5- to 6-membered heterocycle;
   wherein [symbol] represents the attachment point between VLM and LNK,
n^{V} is 0, 1, 2, 3, or 4; and
o^{V} is 0, 1, 2, or 3.

In some embodiments, KTM is a KRAS targeting moiety. In some embodiments, KTM is a KRAS targeting moiety having the structure of formula KTM-I.

In some embodiments, VLM is a Von-Hippel-Lindau (VHL) E3 ubiquitin ligase binding moiety. In some embodiments, VLM is a Von-Hippel-Lindau (VHL) E3 ubiquitin ligase binding moiety having the structure VLM-I.

In some embodiments, KTM has the structure of formula KTM-I:

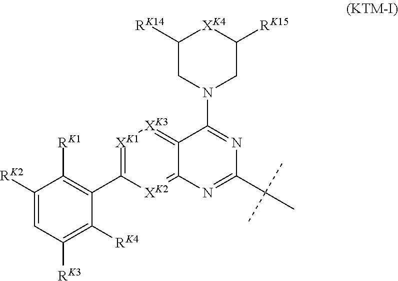

wherein:
X^{K1} is N or CR^{K5};
X^{K2} is N or CR^{K6};
X^{K3} is N or CR^{K7};
X^{K4} is NR^{K8} or C₁-C₃ alkylene, wherein the alkylene is optionally substituted with one or more R^{K9}
R^{K1} and R^{K2} are each independently selected from H, OH, Cl, F, Br, I, C₁-C₆ alkyl, C₁-C₆ haloalkyl, O—(C₁-C₆ alkyl), and O—(C₁-C₆ haloalkyl);
R^{K3} and R^{K4} are each independently selected from H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), and O—(C₁-C₆ haloalkyl); or alternatively,
R^{K3} and R^{K4}, together with the carbons to which they are bonded, form C₆-C₁₀ aryl or 5- to 6-membered heteroaryl, wherein the aryl or heteroaryl is optionally substituted with one, two, three, four, or five R^{K11};
R^{K5}, R^{K6}, and R^{K7} are each independently selected from H, Cl, F, Br, I, NR^{K12}R^{K13}, C₁-C₆ alkyl, and C₁-C₆ haloalkyl;
[symbol] represents the attachment point between KTM and LNK;
R^{K8} and R^{K9} are each independently selected from H, C₁-C₆ alkyl, and C₁-C₆ haloalkyl;
each R^{K11} is independently selected from H, OH, CN, Cl, F, Br, I, NR^{K12}R^{K13} C₁-C₆ alkyl, C₂-C₆ alkenyl, C₂-C₆ alkynyl, and C₁-C₆ haloalkyl;
R^{K12} and R^{K13} are each independently selected from H, C₁-C₆ alkyl, and C₁-C₆ haloalkyl;
R^{K14} and R^{K15} are each independently selected from H, C₁-C₆ alkyl, and C₁-C₆ haloalkyl; or alternatively, R^{K14} and R^{K15}, together with X^{K4} and the carbons to which they are bonded, form a C₄-C₇ cycloalkyl or 4- to 7-membered heterocycle.

In some embodiments, KTM has the structure of formula (KTM-Ia), (KTM-Ib), (KTM-Ic), (KTM-Id), or (KTM-Ie),

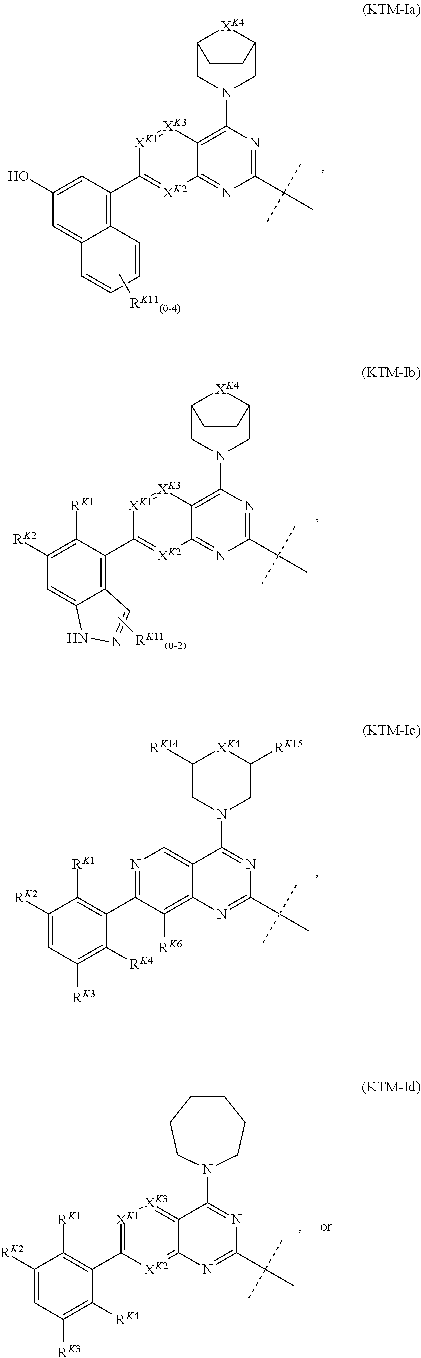

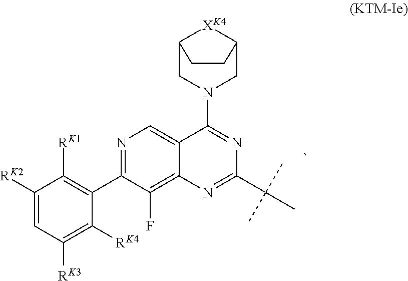

where X^{K1}, X^{K2}, X^{K3}, X^{K4}, R^{K1}, R^{K2}, R^{K3}, R^{K4}, R^{K6}, R^{K11}, R^{K14} and R^{K15} are as defined herein.

In some embodiments KTM has the structure of formula (KTM-Ia). In some embodiments KTM has the structure of formula (KTM-Ib). In some embodiments KTM has the structure of formula (KTM-Ic). In some embodiments KTM has the structure of formula (KTM-Id). In some embodiments KTM has the structure of formula (KTM-Ie).

In some embodiments, X^{K1} is N. In some embodiments X^{K1} is CR^{K5}. In some embodiments X^{K1} is CR^{K5}, and R^{K5} is Cl. In some embodiments X^{K1} is CR^{K5}, and R^{K5} is F. In some embodiments X^{K1} is CR^{K5}, and R^{K5} is Br. In some embodiments X^{K1} is CR^{K5}, and R^{K5} is I. In some embodiments X^{K1} is CR^{K5}, and R^{K5} is NR^{K12}R^{K13}. In some embodiments X^{K1} is CR^{K5}, and R^{K5} is C₁-C₆ alkyl. In some embodiments X^{K1} is CR^{K5}, and R^{K5} is C₁-C₆ haloalkyl.

In some embodiments, R^{K5} is Cl, F, Br, or I. In some embodiments, R^{K5} is C₁-C₆ alkyl or C₁-C₆ haloalkyl.

In some embodiments, R^{K5} is C₁-C₆ alkyl. In some embodiments, R^{K5} is methyl. In some embodiments, R^{K5} is ethyl. In some embodiments, R^{K5} is propyl. In some embodiments, R^{K5} is n-propyl. In some embodiments, R^{K5} is isopropyl. In some embodiments, R^{K5} is butyl. In some embodiments, R^{K5} is n-butyl. In some embodiments, R^{K5} is isobutyl. In some embodiments, R^{K5} is sec-butyl. In some embodiments, R^{K5} is tert-butyl. In some embodiments, R^{K5} is pentyl. In some embodiments, R^{K5} is hexyl.

In some embodiments, R^{K5} is C₁-C₆ haloalkyl. In some embodiments, R^{K5} is C₁ haloalkyl. In some embodiments, R^{K5} is C₂ haloalkyl. In some embodiments, R^{K5} is C₃ haloalkyl. In some embodiments, R^{K5} is C₄ haloalkyl. In some embodiments, R^{K5} is C₅ haloalkyl. In some embodiments, R^{K5} is C₆ haloalkyl.

In some embodiments, X^{K2} is N. In some embodiments X^{K2} is CR^{K6}. In some embodiments X^{K2} is CR^{K6}, and R^{K6} is Cl. In some embodiments X^{K2} is CR^{K6}, and R^{K6} is F. In some embodiments X^{K2} is CR^{K6}, and R^{K6} is Br. In some embodiments X^{K2} is CR^{K6}, and R^{K6} is I. In some embodiments X^{K2} is CR^{K6}, and R^{K6} is NR^{K12}R^{K13}. In some embodiments X^{K2} is CR^{K5}, and R^{K5} is C₁-C₆ alkyl. In some embodiments X^{K2} is CR^{K5}, and R^{K5} is C₁-C₆ haloalkyl.

In some embodiments, R^{K6} is Cl, F, Br, or I. In some embodiments, R^{K6} is C₁-C₆ alkyl or C₁-C₆ haloalkyl.

In some embodiments, R^{K6} is C₁-C₆ alkyl. In some embodiments, R^{K6} is methyl. In some embodiments, R^{K6} is ethyl. In some embodiments, R^{K6} is propyl. In some embodiments, R^{K6} is n-propyl. In some embodiments, R^{K6} is isopropyl. In some embodiments, R^{K6} is butyl. In some embodiments, R^{K6} is n-butyl. In some embodiments, R^{K6} is isobutyl. In some embodiments, R^{K6} is sec-butyl. In some embodiments, R^{K6} is tert-butyl. In some embodiments,

R^{K6} is pentyl. In some embodiments, R^{K6} is hexyl.

In some embodiments, R^{K6} is C₁-C₆ haloalkyl. In some embodiments, R^{K6} is C₁ haloalkyl. In some embodiments, R^{K6} is C₂ haloalkyl. In some embodiments, R^{K6} is C₃ haloalkyl. In some embodiments, R^{K6} is C₄ haloalkyl. In some embodiments, R^{K6} is C₅ haloalkyl. In some embodiments, R^{K6} is C₆ haloalkyl.

In some embodiments, X^{K3} is N. In some embodiments X^{K3} is CR^{K7}. In some embodiments X^{K3} is CR^{K7}, and R^{K7} is Cl. In some embodiments X^{K3} is CR^{K7}, and R^{K7} is F. In some embodiments X^{K3} is CR^{K7}, and R^{K7} is Br. In some embodiments X^{K3} is CR^{K7}, and R^{K7} is I. In some embodiments X^{K3} is CR^{K7}, and R^{K7} is NR^{K12}R^{K13}. In some embodiments X^{K3} is CR^{K7}, and R^{K7} is C₁-C₆ alkyl. In some embodiments X^{K3} is CR^{K7}, and R^{K7} is C₁-C₆ haloalkyl.

In some embodiments, R^{K7} is Cl, F, Br, or I. In some embodiments, R^{K7} is C₁-C₆ alkyl or C₁-C₆ haloalkyl.

In some embodiments, R^{K7} is C₁-C₆ alkyl. In some embodiments, R^{K7} is methyl. In some embodiments, R^{K7} is ethyl. In some embodiments, R^{K7} is propyl. In some embodiments, R^{K7} is n-propyl. In some embodiments, R^{K7} is isopropyl. In some embodiments, R^{K7} is butyl. In some embodiments, R^{K7} is n-butyl. In some embodiments, R^{K7} is isobutyl. In some embodiments, R^{K7} is sec-butyl. In some embodiments, R^{K7} is tert-butyl. In some embodiments, R^{K7} is pentyl. In some embodiments, R^{K7} is hexyl.

In some embodiments, R^{K7} is C₁-C₆ haloalkyl. In some embodiments, R^{K7} is C₁ haloalkyl. In some embodiments, R^{K7} is C₂ haloalkyl. In some embodiments, R^{K7} is C₃ haloalkyl. In some embodiments, R^{K7} is C₄ haloalkyl. In some embodiments, R^{K7} is C₅ haloalkyl. In some embodiments, R^{K7} is C₆ haloalkyl.

In some embodiments, X^{K4} is NR^{K8}. In some embodiments, X^{K4} is NH. In some embodiments, X^{K4} is C₁-C₃ alkylene.

In some embodiments, X^{K4} is unsubstituted C₁-C₃ alkylene. In some embodiments, X^{K4} is C₁-C₃ alkylene substituted with one R^{K9}. In some embodiments, X^{K4} is C₁-C₃ alkylene substituted with two R^{K9}. In some embodiments, X^{K4} is C₁-C₃ alkylene substituted with three R^{K9}.

In some embodiments, X^{K4} is unsubstituted C₁ alkylene (i.e. CH₂). In some embodiments, X^{K4} is unsubstituted C₂ alkylene (i.e. CH₂CH₂). In some embodiments, X^{K4} is unsubstituted C₃ alkylene (i.e. CH₂CH₂CH₂).

In some embodiments, R^{K1} is H, OH, Cl, F, Br, or I. In some embodiments, R^{K1} is Cl, F, Br, or I. In some embodiments, R^{K1} is C₁-C₆ alkyl, C₁-C₆ haloalkyl, O—(C₁-C₆ alkyl), or O—(C₁-C₆ haloalkyl). In some embodiments, R^{K1} is C₁-C₆ alkyl or C₁-C₆ haloalkyl. In some embodiments, R^{K1} is O—(C₁-C₆ alkyl), or O—(C₁-C₆ haloalkyl).

In some embodiments, R^{K1} is H. In some embodiments, R^{K1} is OH. In some embodiments, R^{K1} is F. In some embodiments, R^{K1} is Cl. In some embodiments, R^{K1} is Br. In some embodiments, R^{K1} is I. In some embodiments, R^{K1} is C₁-C₆ alkyl. In some embodiments, R^{K1} is C₁-C₆ haloalkyl. In some embodiments, R^{K1} is O—(C₁-C₆ alkyl). In some embodiments, R^{K1} is O—(C₁-C₆ haloalkyl).

In some embodiments, R^{K1} is methyl. In some embodiments, R^{K1} is ethyl. In some embodiments, R^{K1} is propyl. In some embodiments, R^{K1} is n-propyl. In some embodiments, R^{K1} is isopropyl. In some embodiments, R^{K1} is butyl. In some embodiments, R^{K1} is n-butyl. In some embodiments, R^{K1} is isobutyl. In some embodiments, R^{K1} is sec-butyl. In some embodiments, R^{K1} is tert-butyl. In some embodiments, R^{K1} is pentyl. In some embodiments, R^{K1} is hexyl.

In some embodiments, R^{K1} is C₁ haloalkyl. In some embodiments, R^{K1} is C₂ haloalkyl. In some embodiments, R^{K1} is C₃ haloalkyl. In some embodiments, R^{K1} is C₄ haloalkyl. In some embodiments, R^{K1} is C₅ haloalkyl. In some embodiments, R^{K1} is C₆ haloalkyl.

In some embodiments, R^{K1} is O-methyl. In some embodiments, R^{K1} is O-ethyl. In some embodiments, R^{K1} is O-propyl. In some embodiments, R^{K1} is O-n-propyl. In some embodiments, R^{K1} is O-isopropyl. In some embodiments, R^{K1} is O-butyl. In some embodiments, R^{K1} is O-n-butyl. In some embodiments, R^{K1} is O-isobutyl. In some embodiments, R^{K1} is O-sec-butyl. In some embodiments, R^{K1} is O-tert-butyl. In some embodiments, R^{K1} is O-pentyl. In some embodiments, R^{K1} is O-hexyl.

In some embodiments, R^{K1} is O—C₁ haloalkyl. In some embodiments, R^{K1} is O—C₂ haloalkyl. In some embodiments, R^{K1} is O—C₃ haloalkyl. In some embodiments, R^{K1} is O—C₄ haloalkyl. In some embodiments, R^{K1} is O—C₅ haloalkyl. In some embodiments, R^{K1} is O—C₆ haloalkyl.

In some embodiments, R^{K2} is H, OH, Cl, F, Br, or I. In some embodiments, R^{K2} is Cl, F, Br, or I. In some embodiments, R^{K2} is C₁-C₆ alkyl, C₁-C₆ haloalkyl, O—(C₁-C₆ alkyl), or O—(C₁-C₆ haloalkyl). In some embodiments, R^{K2} is C₁-C₆ alkyl or C₁-C₆ haloalkyl. In some embodiments, R^{K2} is O—(C₁-C₆ alkyl), or O—(C₁-C₆ haloalkyl).

In some embodiments, R^{K2} is H. In some embodiments, R^{K2} is OH. In some embodiments, R^{K2} is F. In some embodiments, R^{K2} is Cl. In some embodiments, R^{K2} is Br. In some embodiments, R^{K2} is I. In some embodiments, R^{K2} is C₁-C₆ alkyl. In some embodiments, R^{K2} is C₁-C₆ haloalkyl. In some embodiments, R^{K2} is O—(C₁-C₆ alkyl). In some embodiments, R^{K2} is O—(C₁-C₆ haloalkyl).

In some embodiments, R^{K2} is methyl. In some embodiments, R^{K2} is ethyl. In some embodiments, R^{K2} is propyl. In some embodiments, R^{K2} is n-propyl. In some embodiments, R^{K2} is isopropyl. In some embodiments, R^{K2} is butyl. In some embodiments, R^{K2} is n-butyl. In some embodiments, R^{K2} is isobutyl. In some embodiments, R^{K2} is sec-butyl. In some embodiments, R^{K2} is tert-butyl. In some embodiments, R^{K2} is pentyl. In some embodiments, R^{K2} is hexyl.

In some embodiments, R^{K2} is C₁ haloalkyl. In some embodiments, R^{K2} is C₂ haloalkyl. In some embodiments, R^{K2} is C₃ haloalkyl. In some embodiments, R^{K2} is C₄ haloalkyl. In some embodiments, R^{K2} is C₅ haloalkyl. In some embodiments, R^{K2} is C₆ haloalkyl.

In some embodiments, R^{K2} is O-methyl. In some embodiments, R^{K2} is O-ethyl. In some embodiments, R^{K2} is O-propyl. In some embodiments, R^{K2} is O-n-propyl. In some embodiments, R^{K2} is O-isopropyl. In some embodiments, R^{K2} is O-butyl. In some embodiments, R^{K2} is O-n-butyl. In some embodiments, R^{K2} is O-isobutyl. In some embodiments, R^{K2} is O-sec-butyl. In some embodiments, R^{K2} is O-tert-butyl. In some embodiments, R^{K2} is O-pentyl. In some embodiments, R^{K2} is O-hexyl.

In some embodiments, R^{K2} is O—C₁ haloalkyl. In some embodiments, R^{K2} is O—C₂ haloalkyl. In some embodiments, R^{K2} is O—C₃ haloalkyl. In some embodiments, R^{K2} is O—C₄ haloalkyl. In some embodiments, R^{K2} is O—C₅ haloalkyl. In some embodiments, R^{K2} is O—C₆ haloalkyl.

In some embodiments, R^{K3} is H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), or O—(C₁-C₆ haloalkyl). In some embodiments, R^{K3} is H, OH, Cl, F, Br, or I. In some embodiments, R^{K3} is Cl, F, Br, or I. In some embodiments, R^{K3} is C₁-C₆ alkyl, C₁-C₆ haloalkyl, O—(C₁-C₆ alkyl), or O—(C₁-C₆ haloalkyl). In some embodiments, R^{K3} is C₁-C₆ alkyl or C₁-C₆ haloalkyl. In some embodiments, R^{K3} is C₁-C₆ haloalkyl or O—(C₁-C₆ haloalkyl). In some embodiments, R^{K3} is CF₃ or O—CF₃. In some embodiments, R^{K3} is O—(C₁-C₆ alkyl) or O—(C₁-C₆ haloalkyl). In some embodiments, R^{K3} is C₃-C₁₀ cycloalkyl or 3- to 10-membered heterocycle.

In some embodiments, R^{K3} is H. In some embodiments, R^{K3} is OH. In some embodiments, R^{K3} is F. In some embodiments, R^{K3} is Cl. In some embodiments, R^{K3} is Br. In some embodiments, R^{K3} is I. In some embodiments, R^{K3} is C₁-C₆ alkyl. In some embodiments, R^{K3} is C₁-C₆ haloalkyl. In some embodiments, R^{K3} is C₃-C₁₀ cycloalkyl. In some embodiments, R^{K3} is C₃-C₁₀ cycloalkyl. In some embodiments, R^{K3} is O—(C₁-C₆ alkyl). In some embodiments, R^{K3} is O—(C₁-C₆ haloalkyl).

In some embodiments, R^{K3} is methyl. In some embodiments, R^{K3} is ethyl. In some embodiments, R^{K3} is propyl. In some embodiments, R^{K3} is n-propyl. In some embodiments, R^{K3} is isopropyl. In some embodiments, R^{K3} is butyl. In some embodiments, R^{K3} is n-butyl. In some embodiments, R^{K3} is isobutyl. In some embodiments, R^{K3} is sec-butyl. In some embodiments, R^{K3} is tert-butyl. In some embodiments, R^{K3} is pentyl. In some embodiments, R^{K3} is hexyl.

In some embodiments, R^{K3} is C₁ haloalkyl. In some embodiments, R^{K3} is C₂ haloalkyl. In some embodiments, R^{K3} is C₃ haloalkyl. In some embodiments, R^{K3} is C₄ haloalkyl. In some embodiments, R^{K3} is C₅ haloalkyl. In some embodiments, R^{K3} is C₆ haloalkyl. In some embodiments, R^{K3} is CF₃.

In some embodiments, R^{K3} is cyclopropyl. In some embodiments, R^{K3} is cyclobutyl. In some embodiments, R^{K3} is cyclopentyl. In some embodiments, R^{K3} is cyclohexyl. In some embodiments, R^{K3} is cycloheptyl. In some embodiments, R^{K3} is cyclooctyl. In some embodiments, R^{K3} is cyclononyl. In some embodiments, R^{K3} is cyclodecyl.

In some embodiments R^{K3} is 3- to 10-membered heterocycle. In some embodiments R^{K3} is 3- to 8-membered heterocycle. In some embodiments R^{K3} is 5- to 9-membered heterocycle.

In some embodiments, R^{K3} is a monocyclic heterocycle. In some embodiments, R^{K3} is a polycyclic heterocycle.

In some embodiments, R^{K3} is 3-membered heterocycle. In some embodiments, R^{K3} is 4-membered heterocycle. In some embodiments, R^{K3} is 5-membered heterocycle. In some embodiments, R^{K3} is 6-membered heterocycle. In some embodiments, R^{K3} is 7-membered heterocycle. In some embodiments, R^{K3} is 8-membered heterocycle. In some embodiments, R^{K3} is 9-membered heterocycle. In some embodiments, R^{K3} is 10-membered heterocycle.

In some embodiments, R^{K3} is O-methyl. In some embodiments, R^{K3} is O-ethyl. In some embodiments, R^{K3} is O-propyl. In some embodiments, R^{K3} is O-n-propyl. In some embodiments, R^{K3} is O-isopropyl. In some embodiments, R^{K3} is O-butyl. In some embodiments, R^{K3} is O-n-butyl. In some embodiments, R^{K3} is O-isobutyl. In some embodiments, R^{K3} is O-sec-butyl. In some embodiments, R^{K3} is O-tert-butyl. In some embodiments, R^{K3} is O-pentyl. In some embodiments, R^{K3} is O-hexyl.

In some embodiments, R^{K3} is O—C₁ haloalkyl. In some embodiments, R^{K3} is O—C₂ haloalkyl. In some embodiments, R^{K3} is O—C₃ haloalkyl. In some embodiments, R^{K3} is O—C₄ haloalkyl. In some embodiments, R^{K3} is O—C₅ haloalkyl. In some embodiments, R^{K3} is O—C₆ haloalkyl.

In some embodiments, R^{K4} is H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), or O—(C₁-C₆ haloalkyl). In some embodiments, R^{K4} is H, OH, Cl, F, Br, or I. In some embodiments, R^{K4} is Cl, F, Br, or I. In some embodiments, R^{K4} is C₁-C₆ alkyl, C₁-C₆ haloalkyl, O—(C₁-C₆ alkyl), or O—(C₁-C₆ haloalkyl). In some embodiments, R^{K4} is C₁-C₆ alkyl or C₁-C₆ haloalkyl. In some embodiments, R^{K4} is C₁-C₆ haloalkyl or O—(C₁-C₆ haloalkyl). In some embodiments, R^{K4} is CF₃ or O—CF₃. In some embodiments, R^{K4} is O—(C₁-C₆ alkyl) or O—(C₁-C₆ haloalkyl). In some embodiments, R^{K4} is C₃-C₁₀ cycloalkyl or 3- to 10-membered heterocycle.

In some embodiments, R^{K4} is H. In some embodiments, R^{K4} is OH. In some embodiments, R^{K4} is F. In some embodiments, R^{K4} is Cl. In some embodiments, R^{K4} is Br. In some embodiments, R^{K4} is I. In some embodiments, R^{K4} is C₁-C₆ alkyl. In some embodiments, R^{K4} is C₁-C₆ haloalkyl. In some embodiments, R^{K4} is C₃-C₁₀ cycloalkyl. In some embodiments, R^{K4} is C₃-C₁₀ cycloalkyl. In some embodiments, R^{K4} is O—(C₁-C₆ alkyl). In some embodiments, R^{K4} is O—(C₁-C₆ haloalkyl).

In some embodiments, R^{K4} is methyl. In some embodiments, R^{K4} is ethyl. In some embodiments, R^{K4} is propyl. In some embodiments, R^{K4} is n-propyl. In some embodiments, R^{K4} is isopropyl. In some embodiments, R^{K4} is butyl. In some embodiments, R^{K4} is n-butyl. In some embodiments, R^{K4} is isobutyl. In some embodiments, R^{K4} is sec-butyl. In some embodiments, R^{K4} is tert-butyl. In some embodiments, R^{K4} is pentyl. In some embodiments,

R^{K4} is hexyl.

In some embodiments, R^{K4} is C₁ haloalkyl. In some embodiments, R^{K4} is C₂ haloalkyl. In some embodiments, R^{K4} is C₃ haloalkyl. In some embodiments, R^{K4} is C₄ haloalkyl. In some embodiments, R^{K4} is C₅ haloalkyl. In some embodiments, R^{K4} is C₆ haloalkyl. In some embodiments, R^{K4} is CF₃.

In some embodiments, R^{K4} is cyclopropyl. In some embodiments, R^{K4} is cyclobutyl. In some embodiments, R^{K4} is cyclopentyl. In some embodiments, R^{K4} is cyclohexyl. In some embodiments, R^{K4} is cycloheptyl. In some embodiments, R^{K4} is cyclooctyl. In some embodiments, R^{K4} is cyclononyl. In some embodiments, R^{K4} is cyclodecyl.

In some embodiments R^{K4} is 3- to 10-membered heterocycle. In some embodiments R^{K4} is 3- to 8-membered heterocycle. In some embodiments R^{K4} is 5- to 9-membered heterocycle.

In some embodiments, R^{K4} is a monocyclic heterocycle. In some embodiments, R^{K4} is a polycyclic heterocycle.

In some embodiments, R^{K4} is 3-membered heterocycle. In some embodiments, R^{K4} is 4-membered heterocycle. In some embodiments, R^{K4} is 5-membered heterocycle. In some embodiments, R^{K4} is 6-membered heterocycle. In some embodiments, R^{K4} is 7-membered heterocycle. In some embodiments, R^{K4} is 8-membered heterocycle. In some embodiments, R^{K4} is 9-membered heterocycle. In some embodiments, R^{K4} is 10-membered heterocycle.

In some embodiments, R^{K4} is O-methyl. In some embodiments, R^{K4} is O-ethyl. In some embodiments, R^{K4} is O-propyl. In some embodiments, R^{K4} is O-n-propyl. In some embodiments, R^{K4} is O-isopropyl. In some embodiments, R^{K4} is O-butyl. In some embodiments, R^{K4} is O-n-butyl. In some embodiments, R^{K4} is O-isobutyl. In some embodiments, R^{K4} is O-sec-butyl. In some embodiments, R^{K4} is O-tert-butyl. In some embodiments, R^{K4} is O-pentyl. In some embodiments, R^{K4} is O-hexyl.

In some embodiments, R^{K4} is O—C₁ haloalkyl. In some embodiments, R^{K4} is O—C₂ haloalkyl. In some embodiments, R^{K4} is O—C₃ haloalkyl. In some embodiments, R^{K4} is O—C₄ haloalkyl. In some embodiments, R^{K4} is O—C₅ haloalkyl. In some embodiments, R^{K4} is O—C₆ haloalkyl.

In some embodiments, R^{K3} and R^{K4}, together with the carbons to which they are bonded, form C₆-C₁₀ aryl or 5- to 6-membered heteroaryl that is unsubstituted.

In some embodiments, R^{K3} and R^{K4}, together with the carbons to which they are bonded, form C₆-C₁₀ aryl or 5- to 6-membered heteroaryl that is substituted with one R^{K11}, wherein R^{K11} is selected from OH, CN, Cl, F, Br, I, NR^{K12}R^{K13}, C₁-C₆ alkyl, C₂-C₆ alkenyl, C₂-C₆ alkynyl, and C₁-C₆ haloalkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is selected from CN, Cl, F, Br, I, and C₁-C₆ alkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is selected from CN, C₁, F, and C₁-C₆ alkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is CN. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is Cl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is F. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is Br. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is I. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is C₁-C₆ alkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is C₁-C₃ alkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is C₁ alkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is C₂ alkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is C₃ alkyl. In some embodiments, R^{K3} and R^{K4}, together with the carbons to which they are bonded, form C₆-C₁₀ aryl or 5- to 6-membered heteroaryl that is substituted with one R^{K11} wherein R^{K11} is C₄ alkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is C₅ alkyl. In some embodiments, R^{K3} and R^{K4}, together with the carbons to which they are bonded, form C₆-C₁₀ aryl or 5- to 6-membered heteroaryl that is substituted with one R^{K11}, wherein R^{K11} is C₆ alkyl.

In some embodiments, R^{K3} and R^{K4}, together with the carbons to which they are bonded, form C₆-C₁₀ aryl that is substituted with one R^{K11}, wherein R^{K11} is selected from CN, Cl, F, Br, I, and C₁-C₆ alkyl. In some embodiments, R^{K3} and R^{K4}, together with the carbons to which they are bonded, form C₆-C₁₀ aryl that is substituted with one R^{K11}, wherein R^{K11} is selected from CN, Cl, F, and C₁-C₆ alkyl.

In some embodiments, R^{K3} and R^{K4}, together with the carbons to which they are bonded, form 5- to 6-membered heteroaryl that is substituted with one R^{K11}, wherein R^{K11} is selected from CN, Cl, F, Br, I, and C₁-C₆ alkyl. In some embodiments, R^{K3} and R^{K4}, together with the carbons to which they are bonded, form 5- to 6-membered heteroaryl that is substituted with one R^{K11}, wherein R^{K11} is selected from CN, Cl, F, and C₁-C₆ alkyl.

In some embodiments, R^{K3} and R^{K4}, together with the carbons to which they are bonded, form C₆-C₁₀ aryl or 5- to 6-membered heteroaryl that is substituted with two R^{K11}, wherein one R^{K11} is selected from OH, CN, Cl, F, Br, I, NR^{K12}R^{K13}, C₁-C₆ alkyl, C₂-C₆ alkenyl, C₂-C₆ alkynyl, and C₁-C₆ haloalkyl, and the other R^{K11} is selected from CN, Cl, F, Br, I, and C₁-C₆ alkyl. In some embodiments, R^{K3} and R^{K4}, together with the carbons to which they are bonded, form C₆-C₁₀ aryl or 5- to 6-membered heteroaryl that is substituted with two R^{K11}, wherein one R^{K11} is selected from OH, CN, Cl, F, Br, I, NR^{K12}R^{K13}, C₁-C₆ alkyl, C₂-C₆ alkenyl, C₂-C₆ alkynyl, and C₁-C₆ haloalkyl, and the other R^{K11} is selected from CN, Cl, F, and C₁-C₆ alkyl.

In some embodiments, R^{K3} and R^{K4}, together with the carbons to which they are bonded, form C₆-C₁₀ aryl or 5- to 6-membered heteroaryl that is substituted with two R^{K11}, wherein both R^{K11} are selected from CN, Cl, F, Br, I, and C₁-C₆ alkyl. In some embodiments, R^{K3} and R^{K4}, together with the carbons to which they are bonded, form C₆-C₁₀ aryl or 5- to 6-membered heteroaryl that is substituted with two R^{K11}, wherein both R^{K11} are selected from CN, Cl, F, and C₁-C₆ alkyl.

In some embodiments, R^{K3} and R^{K4}, together with the carbons to which they are bonded, form C₆-C₁₀ aryl or 5- to 6-membered heteroaryl that is substituted with three R^{K11}, wherein each R^{K11} is independently selected from OH, CN, Cl, F, Br, I, NR^{K12}R^{K13}, C₁-C₆ alkyl, C₂-C₆ alkenyl, C₂-C₆ alkynyl, and C₁-C₆ haloalkyl. In some embodiments, R^{K3} and R^{K4}, together with the carbons to which they are bonded, form C₆-C₁₀ aryl or 5- to 6-membered heteroaryl that is substituted with three R^{K11}, wherein each R^{K11} is independently selected from CN, Cl, F, Br, I, and C₁-C₆ alkyl. In some embodiments, R^{K3} and R^{K4}, together with the carbons to which they are bonded, form C₆-C₁₀ aryl or 5- to 6-membered heteroaryl that is substituted with three R^{K11}, wherein each R^{K11} is independently selected from CN, Cl, F, and C₁-C₆ alkyl.

In some embodiments, R^{K3} and R^{K4}, together with the carbons to which they are bonded, form C₆-C₁₀ aryl or 5- to 6-membered heteroaryl that is substituted with four R^{K11}, wherein each R^{K11} is independently selected from OH, CN, Cl, F, Br, I, NR^{K12}R^{K13}, C₁-C₆ alkyl, C₂-C₆ alkenyl, C₂-C₆ alkynyl, and C₁-C₆ haloalkyl. In some embodiments, R^{K3} and R^{K4}, together with the carbons to which they are bonded, form C₆-C₁₀ aryl or 5- to 6-membered heteroaryl that is substituted with four R^{K11}, wherein each R^{K11} is independently selected from CN, Cl, F, Br, I, and C₁-C₆ alkyl. In some embodiments, R^{K3} and R^{K4}, together with the carbons to which they are bonded, form C₆-C₁₀ aryl or 5- to 6-membered heteroaryl that is substituted with four R^{K11}, wherein each R^{K11} is independently selected from CN, Cl, F, and C₁-C₆ alkyl.

In some embodiments, R^{K3} and R^{K4}, together with the carbons to which they are bonded, form C₆-C₁₀ aryl or 5- to 6-membered heteroaryl that is substituted with five R^{K11}, wherein each R^{K11} is independently selected from OH, CN, Cl, F, Br, I, NR^{K12}R^{K13}, C₁-C₆ alkyl, C₂-C₆ alkenyl, C₂-C₆ alkynyl, and C₁-C₆ haloalkyl. In some embodiments, R^{K3} and R^{K4}, together with the carbons to which they are bonded, form C₆-C₁₀ aryl or 5- to 6-membered heteroaryl that is substituted with five R^{K11}, wherein each R^{K11} is independently selected from CN, Cl, F, Br, I, and C₁-C₆ alkyl. In some embodiments, R^{K3} and R^{K4}, together with the carbons to which they are bonded, form C₆-C₁₀ aryl or 5- to 6-membered heteroaryl that is substituted with five R^{K11}, wherein each R^{K11} is independently selected from CN, Cl, F, and C₁-C₆ alkyl.

In some embodiments, R^{K3} and R^{K4}, together with the carbons to which they are bonded, form C₆-C₁₀ aryl.

In some embodiments, R^{K3} and R^{K4}, together with the carbons to which they are bonded, form C₆ aryl. In some embodiments, R^{K3} and R^{K4}, together with the carbons to which they are bonded, form C₇ aryl. In some embodiments, R^{K3} and R^{K4}, together with the carbons to which they are bonded, form C₈ aryl. In some embodiments, R^{K3} and R^{K4}, together with the carbons to which they are bonded, form C₉ aryl. In some embodiments, R^{K3} and R^{K4}, together with the carbons to which they are bonded, form C₁₀ aryl.

In some embodiments, R^{K3} and R^{K4}, together with the carbons to which they are bonded, form C₆-C₁₀ aryl that is unsubstituted. In some embodiments, R^{K3} and R^{K4}, together with the carbons to which they are bonded, form C₆-C₁₀ aryl that is substituted with one R^{K11}. In some embodiments, R^{K3} and R^{K4}, together with the carbons to which they are bonded, form C₆-C₁₀ aryl that is substituted with two R^{K11}. In some embodiments, R^{K3} and R^{K4}, together with the carbons to which they are bonded, form C₆-C₁₀ aryl that is substituted with three R^{K11}. In some embodiments, R^{K3} and R^{K4}, together with the carbons to which they are bonded, form C₆-C₁₀ aryl that is substituted with four R^{K11}. In some embodiments, R^{K3} and R^{K4}, together with the carbons to which they are bonded, form C₆-C₁₀ aryl that is substituted with five R^{K11}.

In some embodiments, R^{K3} and R^{K4}, together with the carbons to which they are bonded, form 5- or 6-membered heteroaryl.

In some embodiments, R^{K3} and R^{K4}, together with the carbons to which they are bonded, form 5-membered heteroaryl. In some embodiments, R^{K3} and R^{K4}, together with the carbons to which they are bonded, form 6-membered heteroaryl.

In some embodiments, R^{K3} and R^{K4}, together with the carbons to which they are bonded, form 5- or 6-membered heteroaryl that is unsubstituted. In some embodiments, R^{K3} and R^{K4}, together with the carbons to which they are bonded, form 5- or 6-membered heteroaryl that is substituted with one R^{K11}. In some embodiments, R^{K3} and R^{K4}, together with the carbons to which they are bonded, form 5- or 6-membered heteroaryl that is substituted with two R^{K11}. In some embodiments, R^{K3} and R^{K4}, together with the carbons to which they are bonded, form 5- or 6-membered heteroaryl that is substituted with three R^{K11}. In some embodiments, R^{K3} and R^{K4}, together with the carbons to which they are bonded, form 5- or 6-membered heteroaryl that is substituted with four R^{K11}. In some embodiments, R^{K3} and R^{K4}, together with the carbons to which they are bonded, form 5- or 6-membered heteroaryl that is substituted with five R^{K11}.

In some embodiments, R^{K8} is H, C₁-C₆ alkyl, or C₁-C₆ haloalkyl. In some embodiments, R^{K8} is C₁-C₆ alkyl or C₁-C₆ haloalkyl. In some embodiments, R^{K8} is H. In some embodiments, R^{K8} is C₁-C₆ alkyl. In some embodiments, R^{K8} is C₁-C₆ haloalkyl.

In some embodiments, R^{K8} is methyl. In some embodiments, R^{K8} is ethyl. In some embodiments, R^{K8} is propyl. In some embodiments, R^{K8} is n-propyl. In some embodiments, R^{K8} is isopropyl. In some embodiments, R^{K8} is butyl. In some embodiments, R^{K8} is n-butyl. In some embodiments, R^{K8} is isobutyl. In some embodiments, R^{K8} is sec-butyl. In some embodiments, R^{K8} is tert-butyl. In some embodiments, R^{K8} is pentyl. In some embodiments, R^{K8} is hexyl.

In some embodiments, R^{K8} is C₁ haloalkyl. In some embodiments, R^{K8} is C₂ haloalkyl. In some embodiments, R^{K8} is C₃ haloalkyl. In some embodiments, R^{K8} is C₄ haloalkyl. In some embodiments, R^{K8} is C₅ haloalkyl. In some embodiments, R^{K8} is C₆ haloalkyl.

In some embodiments, R^{K9} is H, C₁-C₆ alkyl, or C₁-C₆ haloalkyl. In some embodiments, R^{K9} is C₁-C₆ alkyl or C₁-C₆ haloalkyl. In some embodiments, R^{K9} is H. In some embodiments, R^{K9} is C₁-C₆ alkyl. In some embodiments, R^{K9} is C₁-C₆ haloalkyl.

In some embodiments, R^{K9} is methyl. In some embodiments, R^{K9} is ethyl. In some embodiments, R^{K9} is propyl. In some embodiments, R^{K9} is n-propyl. In some embodiments, R^{K9} is isopropyl. In some embodiments, R^{K9} is butyl. In some embodiments, R^{K9} is n-butyl. In some embodiments, R^{K9} is isobutyl. In some embodiments, R^{K9} is sec-butyl. In some embodiments, R^{K9} is tert-butyl. In some embodiments, R^{K9} is pentyl. In some embodiments, R^{K9} is hexyl.

In some embodiments, R^{K9} is C₁ haloalkyl. In some embodiments, R^{K9} is C₂ haloalkyl. In some embodiments, R^{K9} is C₃ haloalkyl. In some embodiments, R^{K9} is C₄ haloalkyl. In some embodiments, R^{K9} is C₅ haloalkyl. In some embodiments, R^{K9} is C₆ haloalkyl.

In some embodiments, R^{K12} is selected from H, C₁-C₆ alkyl, and C₁-C₆ haloalkyl. In some embodiments, R^{K12} is H. In some embodiments, R^{K12} is selected from C₁-C₆ alkyl, and C₁-C₆ haloalkyl.

In some embodiments, R^{K12} is C₁-C₆ alkyl.

In some embodiments, R^{K12} is methyl. In some embodiments, R^{K12} is ethyl. In some embodiments, R^{K12} is propyl. In some embodiments, R^{K12} is n-propyl. In some embodiments, R^{K12} is isopropyl. In some embodiments, R^{K12} is butyl. In some embodiments, R^{K12} is n-butyl. In some embodiments, R^{K12} is isobutyl. In some embodiments, R^{K12} is sec-butyl. In some embodiments, R^{K12} is tert-butyl. In some embodiments, R^{K12} is pentyl. In some embodiments, R^{K12} is hexyl.

In some embodiments, R^{K12} is C₁-C₆ haloalkyl.

In some embodiments, R^{K12} is C₁ haloalkyl. In some embodiments, R^{K12} is C₂ haloalkyl. In some embodiments, R^{K12} is C₃ haloalkyl. In some embodiments, R^{K12} is C₄ haloalkyl. In some embodiments, R^{K12} is C₅ haloalkyl. In some embodiments, R^{K12} is C₆ haloalkyl.

In some embodiments, R^{K13} is selected from H, C₁-C₆ alkyl, and C₁-C₆ haloalkyl. In some embodiments, R^{K13} is H. In some embodiments, R^{K13} is selected from C₁-C₆ alkyl, and C₁-C₆ haloalkyl.

In some embodiments, R^{K13} is C₁-C₆ alkyl.

In some embodiments, R^{K13} is methyl. In some embodiments, R^{K13} is ethyl. In some embodiments, R^{K13} is propyl. In some embodiments, R^{K13} is n-propyl. In some embodiments, R^{K13} is isopropyl. In some embodiments, R^{K13} is butyl. In some embodiments, R^{K13} is n-butyl. In some embodiments, R^{K13} is isobutyl. In some embodiments, R^{K13} is sec-butyl. In some embodiments, R^{K13} is tert-butyl. In some embodiments, R^{K13} is pentyl. In some embodiments, R^{K13} is hexyl.

In some embodiments, R^{K13} is C₁-C₆ haloalkyl.

In some embodiments, R^{K13} is C₁ haloalkyl. In some embodiments, R^{K13} is C₂ haloalkyl. In some embodiments, R^{K13} is C₃ haloalkyl. In some embodiments, R^{K13} is C₄ haloalkyl. In some embodiments, R^{K13} is C₅ haloalkyl. In some embodiments, R^{K13} is C₆ haloalkyl.

In some embodiments, R^{K12} is H and R^{K13} is selected from C₁-C₆ alkyl, and C₁-C₆ haloalkyl.

In some embodiments, R^{K12} is H and R^{K13} is C₁-C₆ alkyl.

In some embodiments, R^{K12} is H and R^{K13} is methyl. In some embodiments, R^{K12} is H and R^{K13} is ethyl. In some embodiments, R^{K12} is H and R^{K13} is propyl. In some embodiments, R^{K12} is H and R^{K13} is n-propyl. In some embodiments, R^{K12} is H and R^{K13} is isopropyl. In some embodiments, R^{K12} is H and R^{K13} is butyl. In some embodiments, R^{K12} is H and R^{K13} is n-butyl. In some embodiments, R^{K12} is H and R^{K13} is isobutyl. In some embodiments, R^{K12} is H and R^{K13} is sec-butyl. In some embodiments, R^{K12} is H and R^{K13} is tert-butyl. In some embodiments, R^{K12} is H and R^{K13} is pentyl. In some embodiments, R^{K12} is H and R^{K13} is hexyl.

In some embodiments, R^{K12} is H and R^{K13} is C₁-C₆ haloalkyl.

In some embodiments, R^{K12} is H and R^{K13} is C₁ haloalkyl. In some embodiments, R^{K12} is H and R^{K13} is C₂ haloalkyl. In some embodiments, R^{K12} is H and R^{K13} is C₃ haloalkyl. In some embodiments, R^{K12} is H and R^{K13} is C₄ haloalkyl. In some embodiments, R^{K12} is H and R^{K13} is C₅ haloalkyl. In some embodiments, R^{K12} is H and R^{K13} is C₆ haloalkyl.

In some embodiments, R^{K12} is H and R^{K13} is selected from C₁-C₆ alkyl, and C₁-C₆ haloalkyl.

In some embodiments, R^{K12} is H and R^{K13} is C₁-C₆ alkyl.

In some embodiments, R^{K12} is H and R^{K13} is methyl. In some embodiments, R^{K12} is H and R^{K13} is ethyl. In some embodiments, R^{K12} is H and R^{K13} is propyl. In some embodiments, R^{K12} is H and R^{K13} is n-propyl. In some embodiments, R^{K12} is H and R^{K13} is isopropyl. In some embodiments, R^{K12} is H and R^{K13} is butyl. In some embodiments, R^{K12} is H and R^{K13} is n-butyl. In some embodiments, R^{K12} is H and R^{K13} is isobutyl. In some embodiments, R^{K12} is H and R^{K13} is sec-butyl. In some embodiments, R^{K12} is H and R^{K13} is tert-butyl. In some embodiments, R^{K12} is H and R^{K13} is pentyl. In some embodiments, R^{K12} is H and R^{K13} is hexyl.

In some embodiments, R^{K12} is H and R^{K13} is C₁-C₆ haloalkyl.

In some embodiments, R^{K12} is H and R^{K13} is C₁ haloalkyl. In some embodiments, R^{K12} is H and R^{K13} is C₂ haloalkyl. In some embodiments, R^{K12} is H and R^{K13} is C₃ haloalkyl. In some embodiments, R^{K12} is H and R^{K13} is C₄ haloalkyl. In some embodiments, R^{K12} is H and R^{K13} is C₅ haloalkyl. In some embodiments, R^{K12} is H and R^{K13} is C₆ haloalkyl.

In some embodiments, R^{K14} is selected from H, C₁-C₆ alkyl, and C₁-C₆ haloalkyl. In some embodiments, R^{K14} is H. In some embodiments, R^{K14} is selected from C₁-C₆ alkyl, and C₁-C₆ haloalkyl.

In some embodiments, R^{K14} is C₁-C₆ alkyl.

In some embodiments, R^{K14} is methyl. In some embodiments, R^{K14} is ethyl. In some embodiments, R^{K14} is propyl. In some embodiments, R^{K14} is n-propyl. In some embodiments, R^{K14} is isopropyl. In some embodiments, R^{K14} is butyl. In some embodiments, R^{K14} is n-butyl. In some embodiments, R^{K14} is isobutyl. In some embodiments, R^{K14} is sec-butyl. In some embodiments, R^{K14} is tert-butyl. In some embodiments, R^{K14} is pentyl. In some embodiments, R^{K14} is hexyl.

In some embodiments, R^{K14} is C₁-C₆ haloalkyl.

In some embodiments, R^{K14} is C₁ haloalkyl. In some embodiments, R^{K14} is C₂ haloalkyl. In some embodiments, R^{K14} is C₃ haloalkyl. In some embodiments, R^{K14} is C₄ haloalkyl. In some embodiments, R^{K14} is C₅ haloalkyl. In some embodiments, R^{K14} is C₆ haloalkyl.

In some embodiments, R^{K15} is selected from H, C₁-C₆ alkyl, and C₁-C₆ haloalkyl. In some embodiments, R^{K15} is H. In some embodiments, R^{K15} is selected from C₁-C₆ alkyl, and C₁-C₆ haloalkyl.

In some embodiments, R^{K15} is C₁-C₆ alkyl.

In some embodiments, R^{K15} is methyl. In some embodiments, R^{K15} is ethyl. In some embodiments, R^{K15} is propyl. In some embodiments, R^{K15} is n-propyl. In some embodiments, R^{K15} is isopropyl. In some embodiments, R^{K15} is butyl. In some embodiments, R^{K15} is n-butyl. In some embodiments, R^{K15} is isobutyl. In some embodiments, R^{K15} is sec-butyl. In some embodiments, R^{K15} is tert-butyl. In some embodiments, R^{K15} is pentyl. In some embodiments, R^{K15} is hexyl.

In some embodiments, R^{K15} is C₁-C₆ haloalkyl.

In some embodiments, R^{K15} is C₁ haloalkyl. In some embodiments, R^{K15} is C₂ haloalkyl. In some embodiments, R^{K15} is C₃ haloalkyl. In some embodiments, R^{K15} is C₄ haloalkyl. In some embodiments, R^{K15} is C₅ haloalkyl. In some embodiments, R^{K15} is C₆ haloalkyl.

In some embodiments, R^{K14} is H and R^{K15} is selected from C₁-C₆ alkyl, and C₁-C₆ haloalkyl.

In some embodiments, R^{K14} is H and R^{K15} is C₁-C₆ alkyl.

In some embodiments, R^{K14} is H and R^{K15} is methyl. In some embodiments, R^{K14} is H and R^{K15} is ethyl. In some embodiments, R^{K14} is H and R^{K15} is propyl. In some embodiments, R^{K14} is H and R^{K15} is n-propyl. In some embodiments, R^{K14} is H and R^{K15} is isopropyl. In some embodiments, R^{K14} is H and R^{K15} is butyl. In some embodiments, R^{K14} is H and R^{K15} is n-butyl. In some embodiments, R^{K14} is H and R^{K15} is isobutyl. In some embodiments, R^{K14} is H and R^{K15} is sec-butyl. In some embodiments, R^{K14} is H and R^{K15} is tert-butyl. In some embodiments, R^{K14} is H and R^{K15} is pentyl. In some embodiments, R^{K14} is H and R^{K15} is hexyl.

In some embodiments, R^{K14} is H and R^{K15} is C₁-C₆ haloalkyl.

In some embodiments, R^{K14} is H and R^{K15} is C₁ haloalkyl. In some embodiments, R^{K14} is H and R^{K15} is C₂ haloalkyl. In some embodiments, R^{K14} is H and R^{K15} is C₃ haloalkyl. In some embodiments, R^{K14} is H and R^{K15} is C₄ haloalkyl. In some embodiments, R^{K14} is H and R^{K15} is C₅ haloalkyl. In some embodiments, R^{K14} is H and R^{K15} is C₆ haloalkyl.

In some embodiments, R^{K14} is H and R^{K15} is selected from C₁-C₆ alkyl, and C₁-C₆ haloalkyl.

In some embodiments, R^{K14} is H and R^{K15} is C₁-C₆ alkyl.

In some embodiments, R^{K14} is H and R^{K15} is methyl. In some embodiments, R^{K14} is H and R^{K15} is ethyl. In some embodiments, R^{K14} is H and R^{K15} is propyl. In some embodiments, R^{K14} is H and R^{K15} is n-propyl. In some embodiments, R^{K14} is H and R^{K15} is isopropyl. In some embodiments, R^{K14} is H and R^{K15} is butyl. In some embodiments, R^{K14} is H and R^{K15} is n-butyl. In some embodiments, R^{K14} is H and R^{K15} is isobutyl. In some embodiments, R^{K14} is H and R^{K15} is sec-butyl. In some embodiments, R^{K14} is H and R^{K15} is tert-butyl. In some embodiments, R^{K14} is H and R^{K15} is pentyl. In some embodiments, R^{K14} is H and R^{K15} is hexyl.

In some embodiments, R^{K14} is H and R^{K15} is C₁-C₆ haloalkyl.

In some embodiments, R^{K14} is H and R^{K15} is C₁ haloalkyl. In some embodiments, R^{K14} is H and R^{K15} is C₂ haloalkyl. In some embodiments, R^{K14} is H and R^{K15} is C₃ haloalkyl. In some embodiments, R^{K14} is H and R^{K15} is C₄ haloalkyl. In some embodiments, R^{K14} is H and R^{K15} is C₅ haloalkyl. In some embodiments, R^{K14} is H and R^{K15} is C₆ haloalkyl.

In some embodiments, R^{K14} and R^{K15}, together with X^{K4} and the carbons to which they are bonded, form a C₄-C₇ cycloalkyl or 4- to 7-membered heterocycle.

In some embodiments, X^{K4} is C₁-C₃ alkylene and R^{K14} and R^{K15}, together with X^{K4} and the carbons to which they are bonded, form C₄-C₇ cycloalkyl.

In some embodiments, X^{K4} is C₁-C₃ alkylene and R^{K14} and R^{K15}, together with Xk4 and the carbons to which they are bonded, form C₄ a cycloalkyl. In some embodiments, X^{K4} is C₁-C₃ alkylene and R^{K14} and R^{K15}, together with X^{K4} and the carbons to which they are bonded, form C₅ cycloalkyl. In some embodiments, X^{K4} is C₁-C₃ alkylene and R^{K14} and R^{K15}, together with X^{K4} and the carbons to which they are bonded, form C₆ cycloalkyl. In some embodiments, X^{K4} is C₁-C₃ alkylene and R^{K14} and R^{K15}, together with X^{K4} and the carbons to which they are bonded, form C₇ cycloalkyl.

In some embodiments, R^{K14} and R^{K15}, together with X^{K4} and the carbons to which they are bonded, form 4- to 7-membered heterocycle. In some embodiments, R^{K14} and R^{K15}, together with X^{K4} and the carbons to which they are bonded, form 5- or 6-membered heterocycle.

In some embodiments, R^{K14} and R^{K15}, together with X^{K4} and the carbons to which they are bonded, form 4-membered heterocycle. In some embodiments, R^{K14} and R^{K15}, together with X^{K4} and the carbons to which they are bonded, form 5-membered heterocycle. In some embodiments, R^{K14} and R^{K15}, together with X^{K4} and the carbons to which they are bonded, form 6-membered heterocycle. In some embodiments, R^{K14} and R^{K15}, together with X^{K4} and the carbons to which they are bonded, form 7-membered heterocycle.

In some embodiments, wherein KTM has a structure selected from (KTM-1), (KTM-2), (KTM-3), (KTM-4), (KTM-5), (KTM-6), (KTM-7), (KTM-8), (KTM-9), (KTM-10), (KTM-11), (KTM-12), (KTM-13), and (KTM-14):

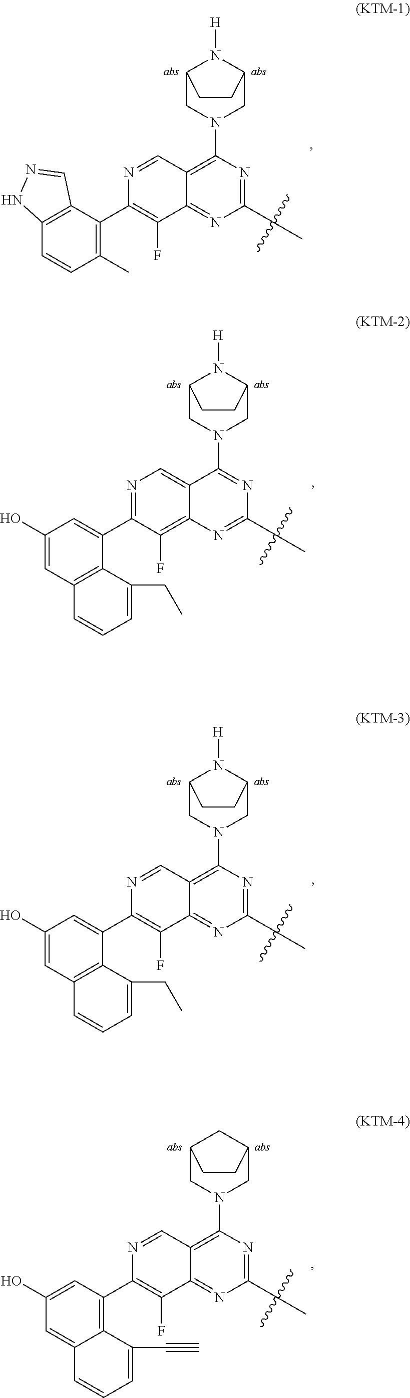 [SMILES: *c1nc(N2CC3CCC(C2)N3)c2cnc(-c3c(C)ccc4[nH]ncc34)c(F)c2n1 ; *c1nc(N2CC3CCC(C2)N3)c2cnc(-c3cc(O)cc4cccc(CC)c34)c(F)c2n1 ; *c1nc(N2CC3CCC(C2)N3)c2cnc(-c3cc(O)cc4cccc(CC)c34)c(F)c2n1 ; *c1nc(N2CC3CCC(C3)C2)c2cnc(-c3cc(O)cc4cccc(C#C)c34)c(F)c2n1]

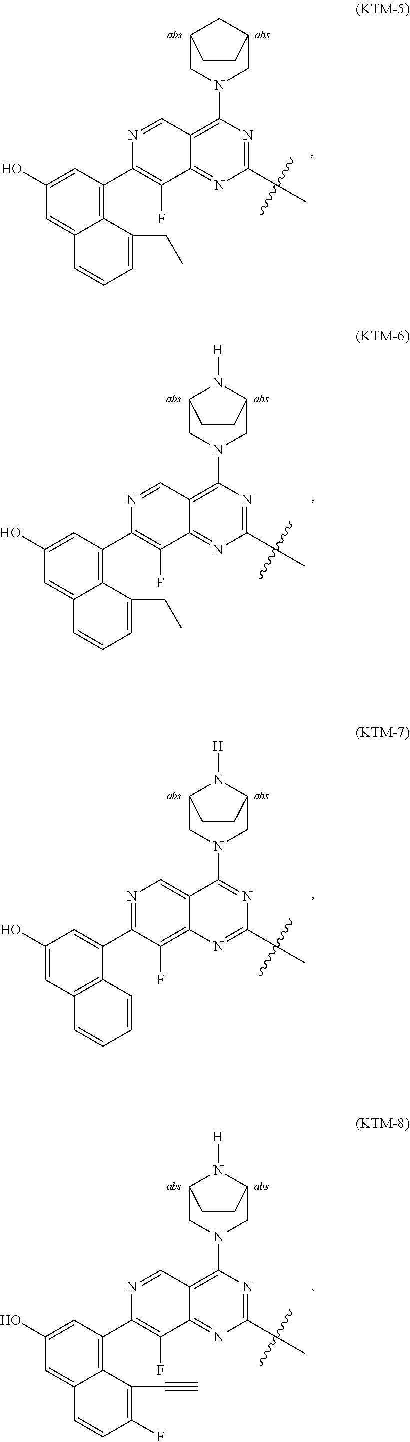 [SMILES: *c1nc(N2CC3CCC(C3)C2)c2cnc(-c3cc(O)cc4cccc(CC)c34)c(F)c2n1 ; *c1nc(N2CC3CCC(C2)N3)c2cnc(-c3cc(O)cc4cccc(CC)c34)c(F)c2n1 ; *c1nc(N2CC3CCC(C2)N3)c2cnc(-c3cc(O)cc4ccccc34)c(F)c2n1 ; *c1nc(N2CC3CCC(C2)N3)c2cnc(-c3cc(O)cc4ccc(F)c(C#C)c34)c(F)c2n1]

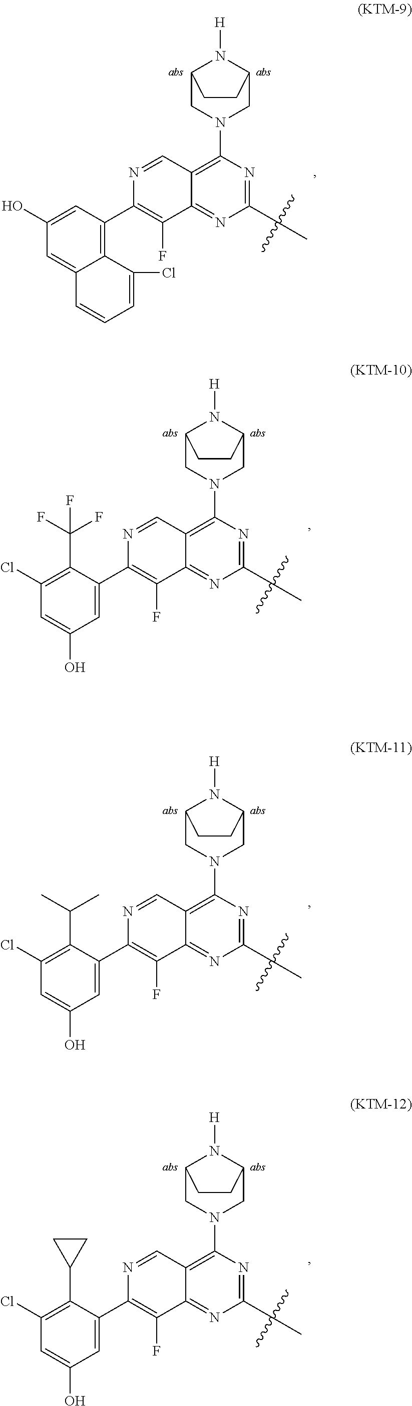 [SMILES: *c1nc(N2CC3CCC(C2)N3)c2cnc(-c3cc(O)cc4cccc(Cl)c34)c(F)c2n1 ; *c1nc(N2CC3CCC(C2)N3)c2cnc(-c3cc(O)cc(Cl)c3C(F)(F)F)c(F)c2n1 ; *c1nc(N2CC3CCC(C2)N3)c2cnc(-c3cc(O)cc(Cl)c3C(C)C)c(F)c2n1 ; *c1nc(N2CC3CCC(C2)N3)c2cnc(-c3cc(O)cc(Cl)c3C3CC3)c(F)c2n1]

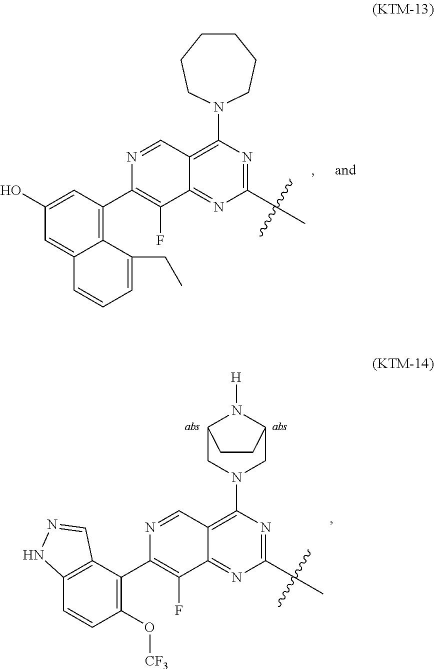 [SMILES: *c1nc(N2CCCCCC2)c2cnc(-c3cc(O)cc4cccc(CC)c34)c(F)c2n1 ; *c1nc(N2CC3CCC(C2)N3)c2cnc(-c3c(OC(F)(F)F)ccc4[nH]ncc34)c(F)c2n1]

In some embodiments, LNK is a chemical linking moiety that covalently couples the KTM to the VLM, having the structure L-I:

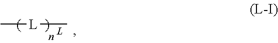

wherein L and n^{L} are as described herein.

In some embodiments, n^{L} is any integer from 1 to 50. In some embodiments, n^{L} is any integer from 1 to 40. In some embodiments, n^{L} is any integer from 1 to 30. In some embodiments, n^{L} is any integer from 1 to 20. In some embodiments, n^{L} is any integer from 1 to 10. In some embodiments, n^{L} is any integer from 1 to 60. In some embodiments, n^{L} is 2, 3, 4, 5, or 6. In some embodiments, n^{L} is 2, 3, 4, or 5. In some embodiments, n^{L} is 2 or 3. In some embodiments, n^{L} is 2. In some embodiments, n^{L} is 3. In some embodiments, n^{L} is 4. In some embodiments, n^{L} is 5. In some embodiments, n^{L} is 6.

In some embodiments, LNK has the structure (L-Ia), (L-Ib), (L-Ic), (L-Id), (L-Ie), or (L-If):

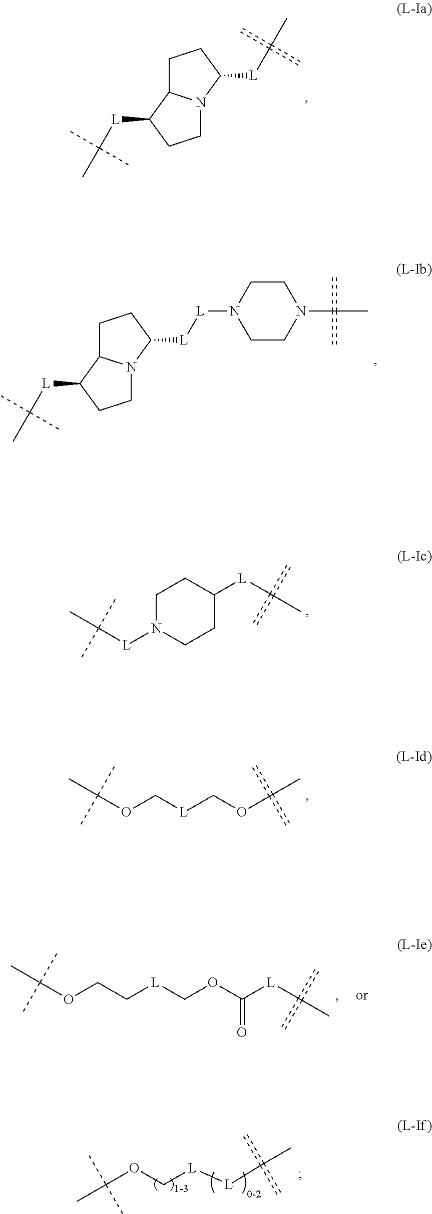

wherein L is as described herein.

In some embodiments, LNK has the structure (L-Ia) or (L-Ib). In some embodiments, LNK has the structure (L-Ia), (L-Ib), or (L-Id). In some embodiments, LNK has the structure (L-Ia), (L-Ib), or (L-Id). In some embodiments, LNK has the structure (L-Ia), (L-Ib), (L-Id), or (L-Ie). In some embodiments, LNK has the structure (L-Ia) or (L-Id). In some embodiments, LNK has the structure (L-Ib) or (L-Ie). In some embodiments, LNK has the structure (L-Ic) or (L-Id). In some embodiments, LNK has the structure (L-Ic), (L-Id), or (I-If). In some embodiments, LNK has the structure (L-Id) or (L-If). In some embodiments, LNK has the structure (L-Ia). In some embodiments, LNK has the structure (L-Ib). In some embodiments, LNK has the structure (L-Ic). In some embodiments, LNK has the structure (L-Id). In some embodiments, LNK has the structure (L-Ie). In some embodiments, LNK has the structure (L-If).

In some embodiments, LNK has the structure (L-Ia′), (L-Ib′), (L-Ic′), (L-Id′), (L-Ie′), or (L-If′):

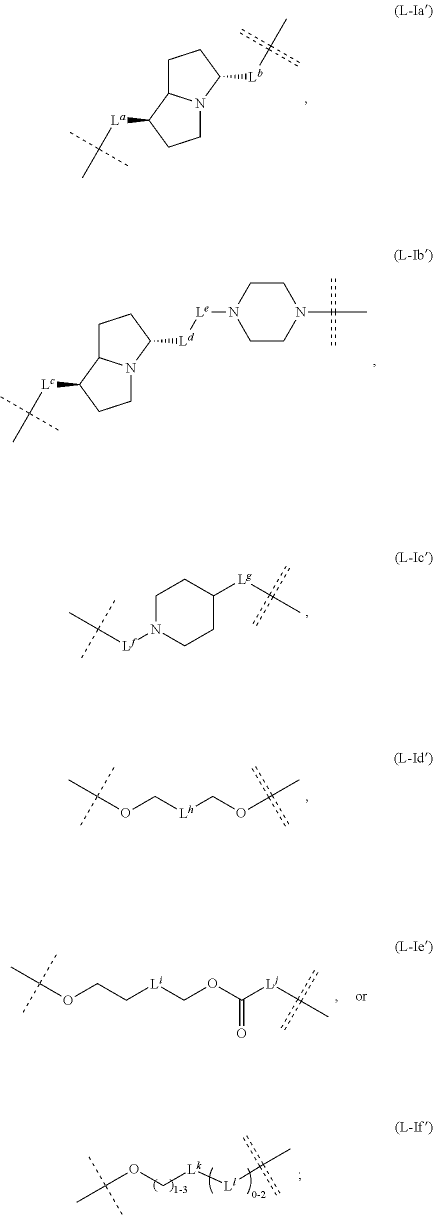

wherein each L^{a}, L^{b}, L^{c}, L^{d}, L^{e}, L^{f}, L^{g}, L^{h}, Lⁱ, L^{j}, L^{k}, and L^{l} is independently absent or selected from

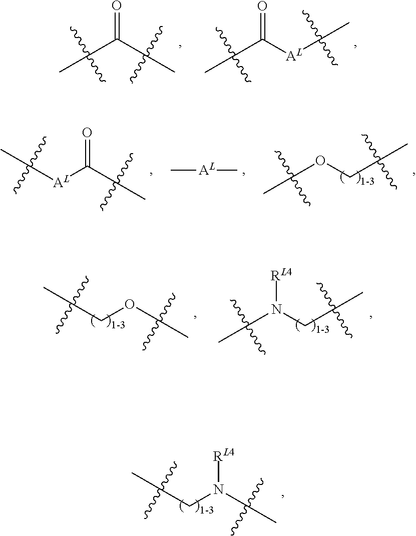

C₂-C₆ alkylene, C₂-C₆ alkenylene, C₂-C₆ alkynylene, monocyclic C₄-C₁₀ cycloalkylene, fused bicyclic C₄-C₁₀ cycloalkylene, bridged bicyclic C₆-C₁₀ cycloalkylene, or spiro-fused bicyclic C₅-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 5-12 membered heterocycloalkylene, C₆-C₁₀ arylene, and 5-6 membered heteroarylene, wherein each cycloalkylene, heterocycloalkylene, arylene, and heteroarylene is optionally substituted with one, two, three, four, or five R^{L5}; wherein A^{L}, R^{L1}, R^{L2}, R^{L3}, R^{L4}, R^{L5}, and n^{L} are as described herein.

In some embodiments, each L^{a}, L^{b}, L^{c}, L^{d}, L^{e}, L^{f}, L^{g}, L^{h}, Lⁱ, L^{j}, L^{k}, and L^{l} is independently selected from

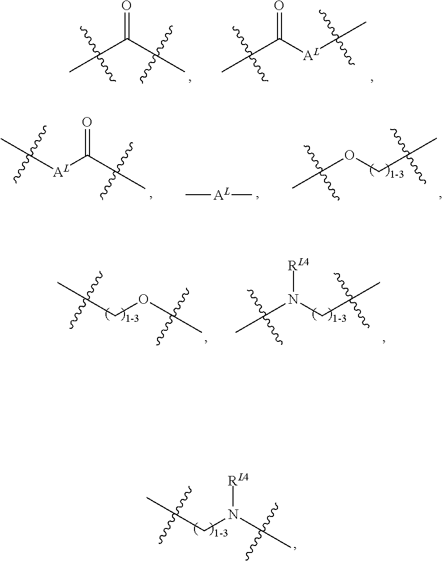

C₂-C₆ alkylene, C₂-C₆ alkenylene, C₂-C₆ alkynylene, monocyclic C₄-C₁₀ cycloalkylene, fused bicyclic C₄-C₁₀ cycloalkylene, bridged bicyclic C₆-C₁₀ cycloalkylene, or spiro-fused bicyclic C₅-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 5-12 membered heterocycloalkylene, C₆-C₁₀ arylene, and 5-6 membered heteroarylene, wherein each cycloalkylene, heterocycloalkylene, arylene, and heteroarylene is optionally substituted with one, two, three, four, or five R^{L5}; wherein A^{L}, R^{L1}, R^{L2}, R^{L3}, R^{L4}, R^{L5}, and n^{L} are as described herein.

In some embodiments, LNK has the structure (L-Ia′) or (L-Ib′). In some embodiments, LNK has the structure (L-Ia′), (L-Ib′), or (L-Id′). In some embodiments, LNK has the structure (L-Ia′), (L-Ib′), or (L-Id′). In some embodiments, LNK has the structure (L-Ia′), (L-Ib′), (L-Id′), or (L-Ie′). In some embodiments, LNK has the structure (L-Ia′) or (L-Id′). In some embodiments, LNK has the structure (L-Ib′) or (L-Ie′). In some embodiments, LNK has the structure (L-Ic′) or (L-Id′). In some embodiments, LNK has the structure (L-Ic′), (L-Id′), or (I-If′). In some embodiments, LNK has the structure (L-Id′) or (L-If′). In some embodiments, LNK has the structure (L-Ia′). In some embodiments, LNK has the structure (L-Ib′). In some embodiments, LNK has the structure (L-Ic′). In some embodiments, LNK has the structure (L-Id′). In some embodiments, LNK has the structure (L-Ie′). In some embodiments, LNK has the structure (L-If′).

In some embodiments, L^{a} is selected from -A^{L}-,

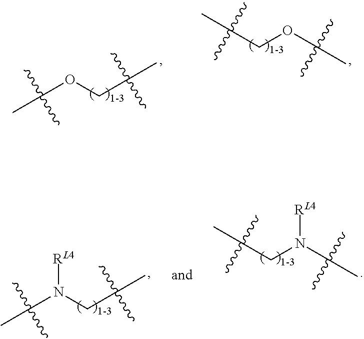

In some embodiments, L^{a} is selected from -A^{L}-,

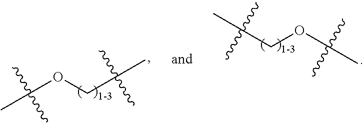

In some embodiments, L^{a} is selected from

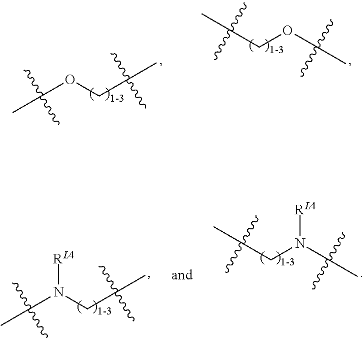

In some embodiments, L^{a} is selected from

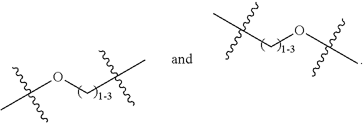

In some embodiments, L^{a} is

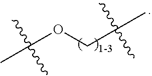

In some embodiments, L^{a} is

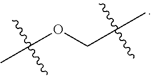 [SMILES: *CO*]

In some embodiments, L^{a} is

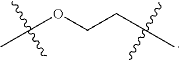 [SMILES: *CCO*]

In some embodiments, L^{a} is

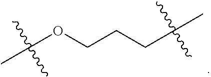 [SMILES: *CCCO*]

In some embodiments, L^{b} is selected from -A^{L}-,

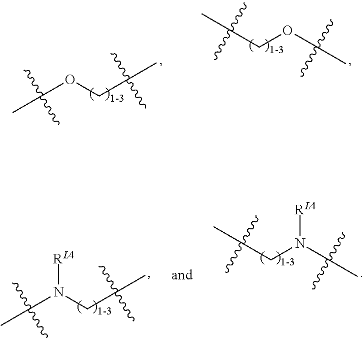

In some embodiments, L^{b} is selected from -A^{L}-,

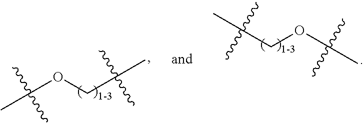

In some embodiments, L^{b} is selected from

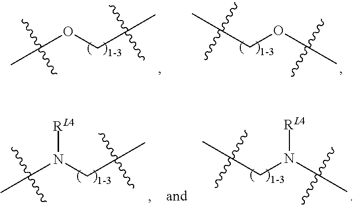

In some embodiments, L^{b} is selected from

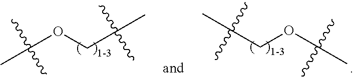

In some embodiments, L^{b} is

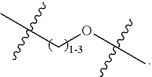

In some embodiments, L^{b} is

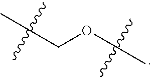 [SMILES: *CO*]

In some embodiments, L^{b} is

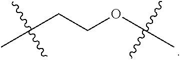 [SMILES: *CCO*]

In some embodiments, L^{b} is

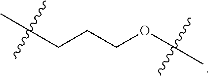 [SMILES: *CCCO*]

In some embodiments, L^{c} is selected from -A^{L}-,

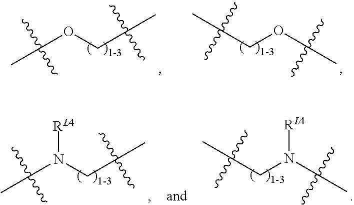

In some embodiments, L^{c} is selected from -A^{L}-,

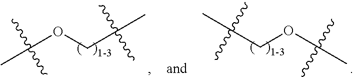

In some embodiments, L^{c} is selected from

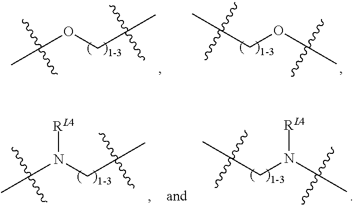

In some embodiments, L^{c} is selected from

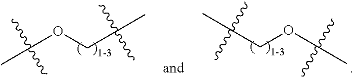

In some embodiments, L^{c} is

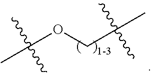

In some embodiments, L^{c} is

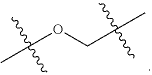 [SMILES: *CO*]

In some embodiments, L^{c} is

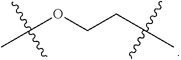 [SMILES: *CCO*]

In some embodiments, L^{c} is

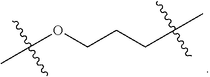 [SMILES: *CCCO*]

In some embodiments, L^{d} is selected from -A^{L}-, C₂-C₆ alkylene, C₂-C₆ alkenylene, C₂-C₆ alkynylene. In some embodiments, L^{d} is -A^{L}-. In some embodiments, L^{d} is selected from C₂-C₆ alkylene. In some embodiments, L^{d} is selected from C₂-C₆ alkenylene. In some embodiments, L^{d} is selected from C₂-C₆ alkynylene. In some embodiments, L^{d} is —CH₂—. In some embodiments, L^{d} is —CH₂CH₂—. In some embodiments, L^{d} is —CH₂CH₂CH₂—. In some embodiments, L^{d} is —CH₂CH₂CH₂CH₂—. In some embodiments, L^{d} is —CH₂CH₂CH₂CH₂CH₂—. In some embodiments, L^{d} is —CH₂CH₂CH₂CH₂CH₂CH₂—.

In some embodiments L^{e} is selected from

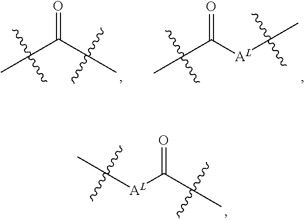

and -A^{L}-. In some embodiments L^{e} is selected from

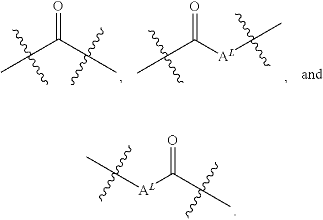

In some embodiments L^{e} is selected from

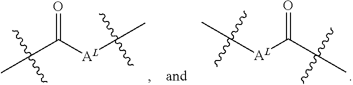

In some embodiments L^{e} is selected from

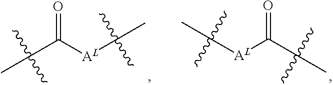

and -A^{L}-. In some embodiments L^{e} is selected from

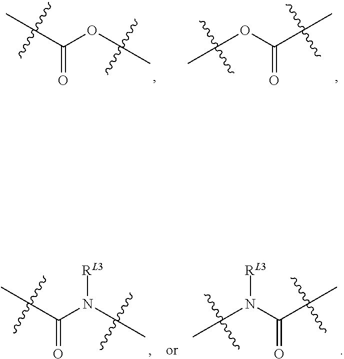

In some embodiments L^{e} is selected from

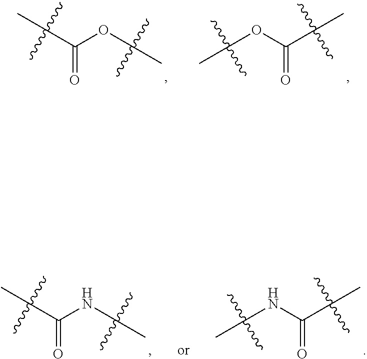 [SMILES: *OC(*)=O ; *OC(*)=O ; *NC(*)=O ; *NC(*)=O]

In some embodiments L^{e} is selected from

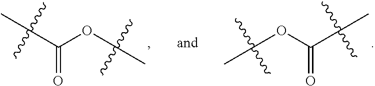 [SMILES: *OC(*)=O ; *OC(*)=O]

In some embodiments L^{e} is

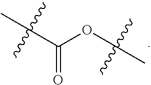 [SMILES: *OC(*)=O]

In some embodiments L^{e} is

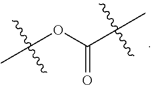 [SMILES: *OC(*)=O]

In some embodiments, L^{f} is selected from -A^{L}-,

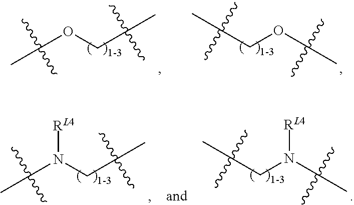

In some embodiments, L^{f} is selected from -A^{L}-,

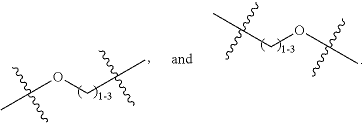

In some embodiments, L^{f} is selected from

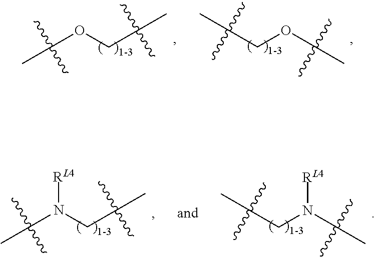

In some embodiments, L^{f} is selected from

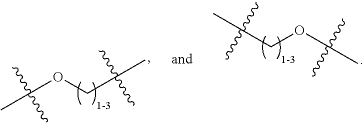

In some embodiments, L^{f} is

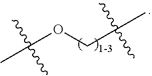

In some embodiments, L^{f} is

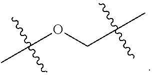 [SMILES: *CO*]

In some embodiments, L^{f} is

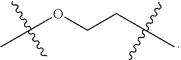 [SMILES: *CCO*]

In some embodiments, L^{f} is

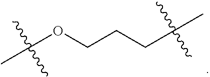 [SMILES: *CCCO*]

In some embodiments, L^{g} is selected from -A^{L}-,

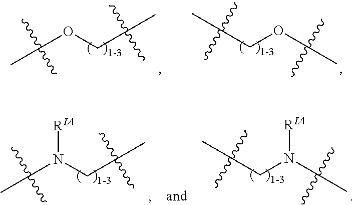

In some embodiments, L^{g} is selected from -A^{L}-,

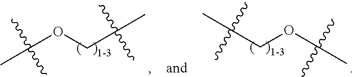

In some embodiments, L^{g} is selected from

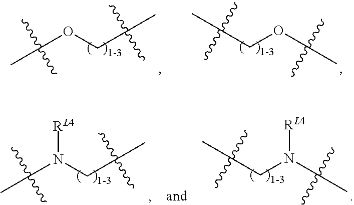

In some embodiments, L^{g} is selected from

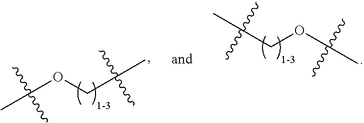

In some embodiments, L^{g} is

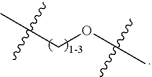

In some embodiments, L^{g} is

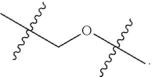 [SMILES: *CO*]

In some embodiments, L^{g} is

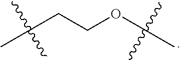 [SMILES: *CCO*]

In some embodiments, L^{g} is

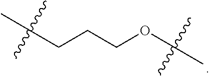 [SMILES: *CCCO*]

In some embodiments, L^{h} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene, C₆-C₁₀ arylene, and 5-6 membered heteroarylene, wherein L^{h} is optionally substituted with one, two, three, four, or five R^{L5}. In some embodiments, L^{h} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene, C₆-C₁₀ arylene, and 5-6 membered heteroarylene, wherein L^{h} is unsubstituted.

In some embodiments, L^{h} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene. In some embodiments, L^{h} is selected from monocyclic 4-10 membered heterocycloalkylene, fused bicyclic 4-10 membered heterocycloalkylene, bridged bicyclic 6-membered heterocycloalkylene, spiro-fused 4-12 membered heterocycloalkylene. In some embodiments, L^{h} is selected from C₆-C₁₀ arylene and 5-6 membered heteroarylene.

In some embodiments, L^{h} is selected from monocyclic 4-10 membered heterocycloalkylene. In some embodiments, L^{h} is selected from fused bicyclic 4-10 membered heterocycloalkylene. In some embodiments, L^{h} is selected from bridged bicyclic 6-10 membered heterocycloalkylene. In some embodiments, L^{h} is selected from spiro-fused bicyclic 4-12 membered heterocycloalkylene.

In some embodiments, L^{h} is monocyclic 4- to 10-membered heterocycloalkylene. In some embodiments, L^{h} is monocyclic 4- to 7-membered heterocycloalkylene. In some embodiments, L^{h} is monocyclic 5- or 6-membered heterocycloalkylene.

In some embodiments, L^{h} is monocyclic 4-membered heterocycloalkylene. In some embodiments, L^{h} is monocyclic 5-membered heterocycloalkylene. In some embodiments, L^{h} is monocyclic 6-membered heterocycloalkylene. In some embodiments, L^{h} is monocyclic 7-membered heterocycloalkylene. In some embodiments, L^{h} is monocyclic 8-membered heterocycloalkylene. In some embodiments, L^{h} is monocyclic 9-membered heterocycloalkylene. In some embodiments, L^{h} is monocyclic 10-membered heterocycloalkylene.

In some embodiments, L^{h} is fused bicyclic 6- to 10-membered heterocycloalkylene. In some embodiments, L^{h} is fused bicyclic 8- to 10-membered heterocycloalkylene. In some embodiments, L^{h} is fused bicyclic 5- or 6-membered heterocycloalkylene.

In some embodiments, L^{h} is fused bicyclic 4-membered heterocycloalkylene. In some embodiments, L^{h} is fused bicyclic 5-membered heterocycloalkylene. In some embodiments, L^{h} is fused bicyclic 6-membered heterocycloalkylene. In some embodiments, L^{h} is fused bicyclic 7-membered heterocycloalkylene. In some embodiments, L^{h} is fused bicyclic 8-membered heterocycloalkylene. In some embodiments, L^{h} is fused bicyclic 9-membered heterocycloalkylene. In some embodiments, L^{h} is fused bicyclic 10-membered heterocycloalkylene.

In some embodiments, L^{h} is bridged bicyclic 6- to 10-membered heterocycloalkylene. In some embodiments, L^{h} is bridged bicyclic 6 or 7-membered heterocycloalkylene.

In some embodiments, L^{h} is bridged bicyclic 6-membered heterocycloalkylene. In some embodiments, L^{h} is bridged bicyclic 7-membered heterocycloalkylene. In some embodiments, L^{h} is bridged bicyclic 8-membered heterocycloalkylene. In some embodiments, L^{h} is bridged bicyclic 9-membered heterocycloalkylene. In some embodiments, L^{h} is bridged bicyclic 10-membered heterocycloalkylene.

In some embodiments, L^{h} is spiro-fused bicyclic 4- to 12-membered heterocycloalkylene. In some embodiments, L^{h} is spiro-fused bicyclic 7- to 11-membered heterocycloalkylene. In some embodiments, L^{h} is spiro-fused bicyclic 7- or 8-membered heterocycloalkylene.

In some embodiments, L^{h} is spiro-fused bicyclic 4-membered heterocycloalkylene. In some embodiments, L^{h} is spiro-fused bicyclic 5-membered heterocycloalkylene. In some embodiments, L^{h} is spiro-fused bicyclic 6-membered heterocycloalkylene. In some embodiments, L^{h} is spiro-fused bicyclic 7-membered heterocycloalkylene. In some embodiments, L^{h} is spiro-fused bicyclic 8-membered heterocycloalkylene. In some embodiments, L^{h} is spiro-fused bicyclic 9-membered heterocycloalkylene. In some embodiments, L^{h} is spiro-fused bicyclic 10-membered heterocycloalkylene. In some embodiments, L^{h} is spiro-fused bicyclic 11-membered heterocycloalkylene. In some embodiments, L^{h} is spiro-fused bicyclic 12-membered heterocycloalkylene.

In some embodiments, L^{h} is selected from

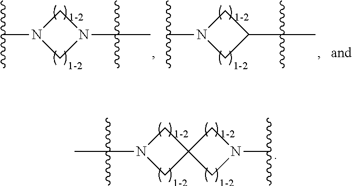

In some embodiments, L^{h} is selected from

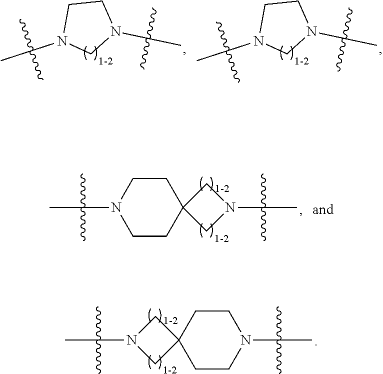

In some embodiments, L^{h} is selected from

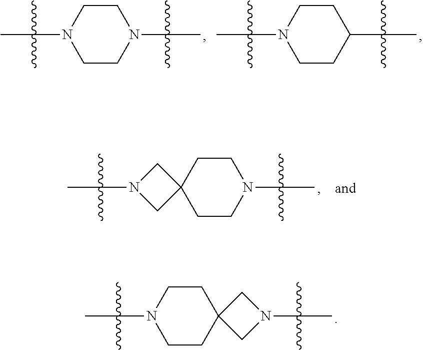 [SMILES: *N1CCN(*)CC1 ; *C1CCN(*)CC1 ; *N1CCC2(CC1)CN(*)C2 ; *N1CCC2(CC1)CN(*)C2]

In some embodiments, L^{h} is selected from

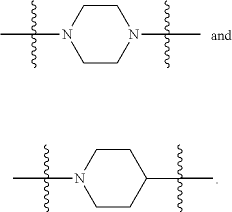 [SMILES: *N1CCN(*)CC1 ; *C1CCN(*)CC1]

In some embodiments, L^{h} is selected from

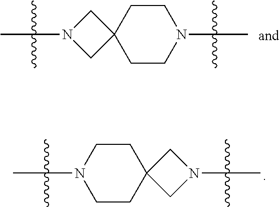 [SMILES: *N1CCC2(CC1)CN(*)C2 ; *N1CCC2(CC1)CN(*)C2]

In some embodiments, L^{h} is

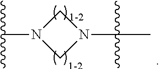

In some embodiments, L^{h} is

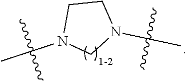

In some embodiments, L^{h} is

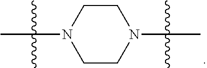 [SMILES: *N1CCN(*)CC1]

In some embodiments, L^{h} is

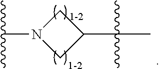

In some embodiments, L^{h} is

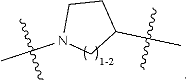

In some embodiments, L^{h} is

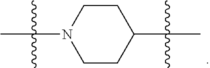 [SMILES: *C1CCN(*)CC1]

In some embodiments, L^{h} is

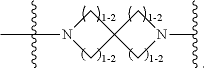

In some embodiments, L^{h} is

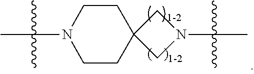

In some embodiments, L^{h} is. In some embodiments, L^{h} is

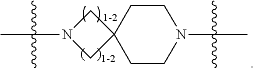

In some embodiments, L^{h} is

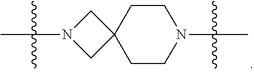 [SMILES: *N1CCC2(CC1)CN(*)C2]

In some embodiments, L^{h} is

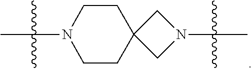 [SMILES: *N1CCC2(CC1)CN(*)C2]

In some embodiments, Lⁱ is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene, C₆-C₁₀ arylene, and 5-6 membered heteroarylene, wherein Lⁱ is optionally substituted with one, two, three, four, or five R^{L5}. In some embodiments, Lⁱ is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene, C₆-C₁₀ arylene, and 5-6 membered heteroarylene, wherein Lⁱ is unsubstituted.

In some embodiments, Lⁱ is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene. In some embodiments, Lⁱ is selected from monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene. In some embodiments, Lⁱ is selected from C₆-C₁₀ arylene and 5-6 membered heteroarylene.

In some embodiments, Lⁱ is selected from monocyclic 4-10 membered heterocycloalkylene. In some embodiments, Lⁱ is selected from fused bicyclic 4-10 membered heterocycloalkylene. In some embodiments, Lⁱ is selected from bridged bicyclic 6-10 membered heterocycloalkylene. In some embodiments, Lⁱ is selected from spiro-fused bicyclic 4-12 membered heterocycloalkylene.

In some embodiments, Lⁱ is monocyclic 4- to 10-membered heterocycloalkylene. In some embodiments, Lⁱ is monocyclic 4- to 7-membered heterocycloalkylene. In some embodiments, Lⁱ is monocyclic 5- or 6-membered heterocycloalkylene.

In some embodiments, Lⁱ is monocyclic 4-membered heterocycloalkylene. In some embodiments, Lⁱ is monocyclic 5-membered heterocycloalkylene. In some embodiments, Lⁱ is monocyclic 6-membered heterocycloalkylene. In some embodiments, Lⁱ is monocyclic 7-membered heterocycloalkylene. In some embodiments, Lⁱ is monocyclic 8-membered heterocycloalkylene. In some embodiments, Lⁱ is monocyclic 9-membered heterocycloalkylene. In some embodiments, Lⁱ is monocyclic 10-membered heterocycloalkylene.

In some embodiments, Lⁱ is fused bicyclic 6- to 10-membered heterocycloalkylene. In some embodiments, Lⁱ is fused bicyclic 8- to 10-membered heterocycloalkylene. In some embodiments, Lⁱ is fused bicyclic 5- or 6-membered heterocycloalkylene.

In some embodiments, Lⁱ is fused bicyclic 4-membered heterocycloalkylene. In some embodiments, Lⁱ is fused bicyclic 5-membered heterocycloalkylene. In some embodiments, Lⁱ is fused bicyclic 6-membered heterocycloalkylene. In some embodiments, Lⁱ is fused bicyclic 7-membered heterocycloalkylene. In some embodiments, Lⁱ is fused bicyclic 8-membered heterocycloalkylene. In some embodiments, Lⁱ is fused bicyclic 9-membered heterocycloalkylene. In some embodiments, Lⁱ is fused bicyclic 10-membered heterocycloalkylene.

In some embodiments, Lⁱ is bridged bicyclic 6- to 10-membered heterocycloalkylene. In some embodiments, Lⁱ is bridged bicyclic 6 or 7-membered heterocycloalkylene.

In some embodiments, Lⁱ is bridged bicyclic 6-membered heterocycloalkylene. In some embodiments, Lⁱ is bridged bicyclic 7-membered heterocycloalkylene. In some embodiments, Lⁱ is bridged bicyclic 8-membered heterocycloalkylene. In some embodiments, Lⁱ is bridged bicyclic 9-membered heterocycloalkylene. In some embodiments, Lⁱ is bridged bicyclic 10-membered heterocycloalkylene.

In some embodiments, Lⁱ is spiro-fused bicyclic 4- to 12-membered heterocycloalkylene. In some embodiments, Lⁱ is spiro-fused bicyclic 7- to 11-membered heterocycloalkylene. In some embodiments, Lⁱ is spiro-fused bicyclic 7- or 8-membered heterocycloalkylene.

In some embodiments, Lⁱ is spiro-fused bicyclic 4-membered heterocycloalkylene. In some embodiments, Lⁱ is spiro-fused bicyclic 5-membered heterocycloalkylene. In some embodiments, Lⁱ is spiro-fused bicyclic 6-membered heterocycloalkylene. In some embodiments, Lⁱ is spiro-fused bicyclic 7-membered heterocycloalkylene. In some embodiments, Lⁱ is spiro-fused bicyclic 8-membered heterocycloalkylene. In some embodiments, is spiro-fused bicyclic 9-membered heterocycloalkylene. In some Lⁱ embodiments, Lⁱ is spiro-fused bicyclic 10-membered heterocycloalkylene. In some embodiments, Lⁱ is spiro-fused bicyclic 11-membered heterocycloalkylene. In some embodiments, Lⁱ is spiro-fused bicyclic 12-membered heterocycloalkylene.

In some embodiments, Lⁱ is selected from

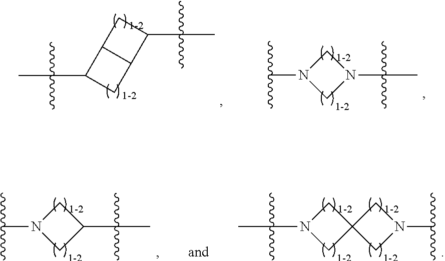

In some embodiments, Lⁱ is selected from

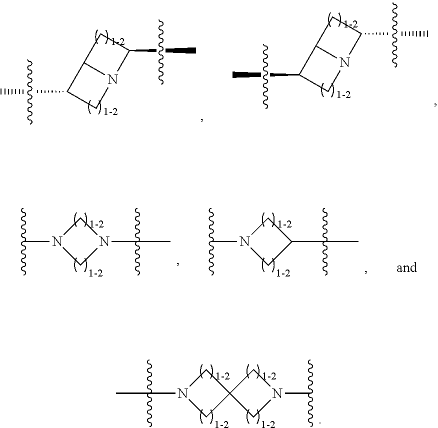

In some embodiments, Lⁱ is selected from

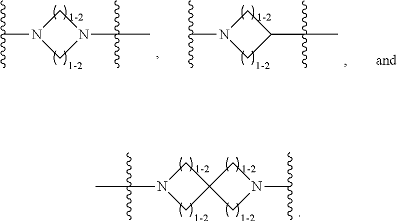

In some embodiments, Lⁱ is selected from

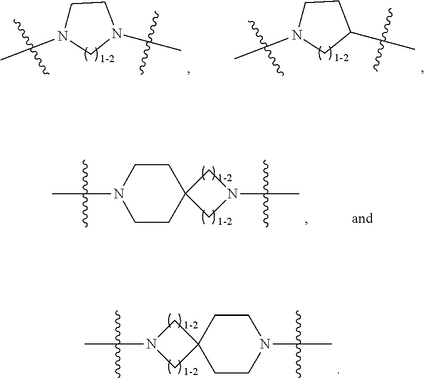

In some embodiments, Lⁱ is selected from

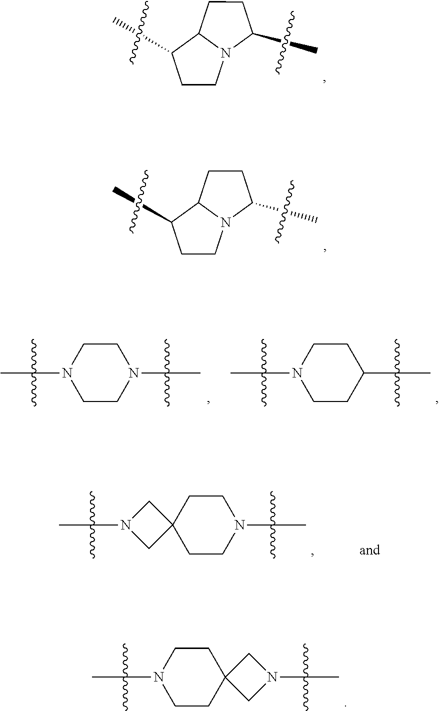

In some embodiments, Lⁱ is selected from

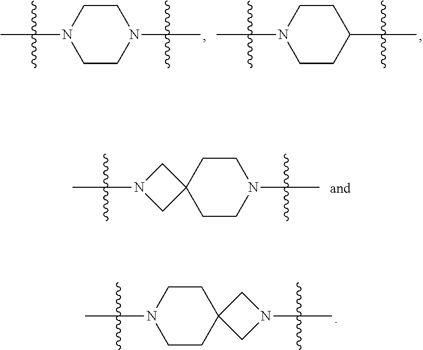 [SMILES: *N1CCN(*)CC1 ; *C1CCN(*)CC1 ; *N1CCC2(CC1)CN(*)C2 ; *N1CCC2(CC1)CN(*)C2]

In some embodiments, Lⁱ is selected from

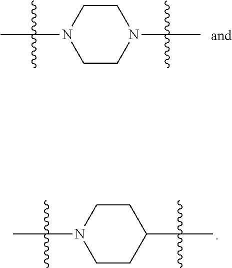 [SMILES: *N1CCN(*)CC1 ; *C1CCN(*)CC1]

In some embodiments, Lⁱ is selected from

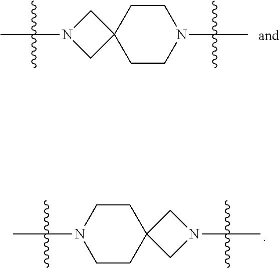 [SMILES: *N1CCC2(CC1)CN(*)C2 ; *N1CCC2(CC1)CN(*)C2]

In some embodiments, Lⁱ is

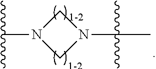

In some embodiments, Lⁱ is

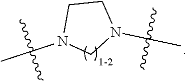

In some embodiments, Lⁱ is

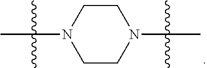 [SMILES: *N1CCN(*)CC1]

In some embodiments, Lⁱ is

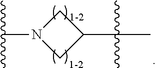

In some embodiments, Lⁱ is

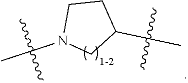

In some embodiments, Lⁱ is

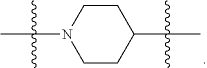 [SMILES: *C1CCN(*)CC1]

In some embodiments, Lⁱ is

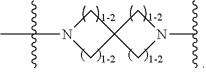

In some embodiments, Lⁱ is

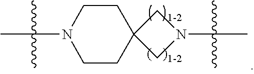

In some embodiments, Lⁱ is. In some embodiments, Lⁱ is

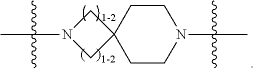

In some embodiments, Lⁱ is

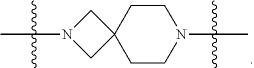 [SMILES: *N1CCC2(CC1)CN(*)C2]

In some embodiments, Lⁱ is

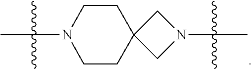 [SMILES: *N1CCC2(CC1)CN(*)C2]

In some embodiments, Lⁱ is

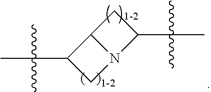

In some embodiments, Lⁱ is

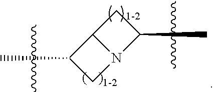

In some embodiments, Lⁱ is

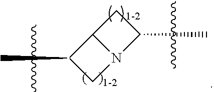

In some embodiments, Lⁱ is

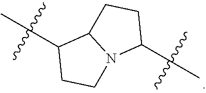 [SMILES: *C1CCN2C(*)CCC12]

In some embodiments, Lⁱ is

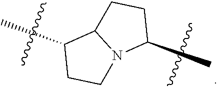

In some embodiments, Lⁱ is

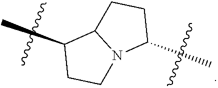

In some embodiments, L^{j} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene, C₆-C₁₀ arylene, and 5-6 membered heteroarylene, wherein L^{j} is optionally substituted with one, two, three, four, or five R^{L5}. In some embodiments, L^{j} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene, C₆-C₁₀ arylene, and 5-6 membered heteroarylene, wherein L^{j} is unsubstituted.

In some embodiments, L^{j} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene. In some embodiments, L^{j} is selected from monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene. In some embodiments, L^{j} is selected from C₆-C₁₀ arylene and 5-6 membered heteroarylene.

In some embodiments, L^{j} is selected from monocyclic 4-10 membered heterocycloalkylene. In some embodiments, L^{j} is selected from fused bicyclic 4-10 membered heterocycloalkylene. In some embodiments, L^{j} is selected from bridged bicyclic 6-10 membered heterocycloalkylene. In some embodiments, L^{j} is selected from spiro-fused bicyclic 4-12 membered heterocycloalkylene.

In some embodiments, L^{j} is monocyclic 4- to 10-membered heterocycloalkylene. In some embodiments, L^{j} is monocyclic 4- to 7-membered heterocycloalkylene. In some embodiments, L^{j} is monocyclic 5- or 6-membered heterocycloalkylene.

In some embodiments, L^{j} is monocyclic 4-membered heterocycloalkylene. In some embodiments, L^{j} is monocyclic 5-membered heterocycloalkylene. In some embodiments, L^{j} is monocyclic 6-membered heterocycloalkylene. In some embodiments, L^{j} is monocyclic 7-membered heterocycloalkylene. In some embodiments, L^{j} is monocyclic 8-membered heterocycloalkylene. In some embodiments, L^{j} is monocyclic 9-membered heterocycloalkylene. In some embodiments, L^{j} is monocyclic 10-membered heterocycloalkylene.

In some embodiments, L^{j} is fused bicyclic 6- to 10-membered heterocycloalkylene. In some embodiments, L^{j} is fused bicyclic 8- to 10-membered heterocycloalkylene. In some embodiments, L^{j} is fused bicyclic 5- or 6-membered heterocycloalkylene.

In some embodiments, L^{j} is fused bicyclic 4-membered heterocycloalkylene. In some embodiments, L^{j} is fused bicyclic 5-membered heterocycloalkylene. In some embodiments, L^{j} is fused bicyclic 6-membered heterocycloalkylene. In some embodiments, L^{j} is fused bicyclic 7-membered heterocycloalkylene. In some embodiments, L^{j} is fused bicyclic 8-membered heterocycloalkylene. In some embodiments, L^{j} is fused bicyclic 9-membered heterocycloalkylene. In some embodiments, L^{j} is fused bicyclic 10-membered heterocycloalkylene.

In some embodiments, L^{j} is bridged bicyclic 6- to 10-membered heterocycloalkylene. In some embodiments, L^{j} is bridged bicyclic 6 or 7-membered heterocycloalkylene.

In some embodiments, L^{j} is bridged bicyclic 6-membered heterocycloalkylene. In some embodiments, L^{j} is bridged bicyclic 7-membered heterocycloalkylene. In some embodiments, L^{j} is bridged bicyclic 8-membered heterocycloalkylene. In some embodiments, L^{j} is bridged bicyclic 9-membered heterocycloalkylene. In some embodiments, L^{j} is bridged bicyclic 10-membered heterocycloalkylene.

In some embodiments, L^{j} is spiro-fused bicyclic 4- to 12-membered heterocycloalkylene. In some embodiments, L^{j} is spiro-fused bicyclic 7- to 11-membered heterocycloalkylene. In some embodiments, L^{j} is spiro-fused bicyclic 7- or 8-membered heterocycloalkylene.

In some embodiments, L^{j} is spiro-fused bicyclic 4-membered heterocycloalkylene. In some embodiments, L^{j} is spiro-fused bicyclic 5-membered heterocycloalkylene. In some embodiments, L^{j} is spiro-fused bicyclic 6-membered heterocycloalkylene. In some embodiments, L^{j} is spiro-fused bicyclic 7-membered heterocycloalkylene. In some embodiments, is spiro-fused bicyclic 8-membered heterocycloalkylene. In some L^{j} embodiments, L^{j} is spiro-fused bicyclic 9-membered heterocycloalkylene. In some embodiments, L^{j} is spiro-fused bicyclic 10-membered heterocycloalkylene. In some embodiments, L^{j} is spiro-fused bicyclic 11-membered heterocycloalkylene. In some embodiments, L^{j} is spiro-fused bicyclic 12-membered heterocycloalkylene.

In some embodiments, L^{j} is selected from

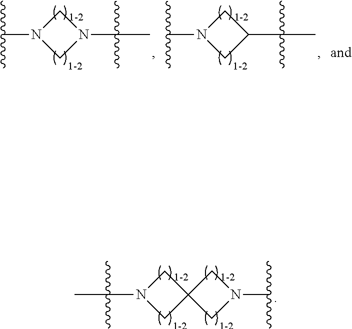

In some embodiments, L^{j} is selected from

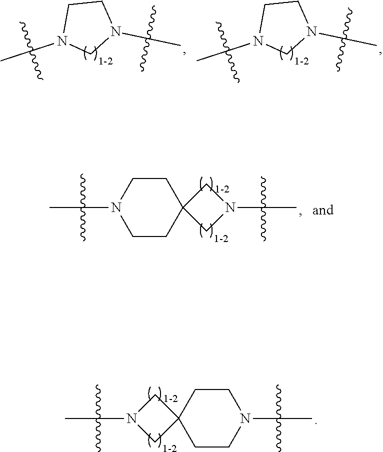

In some embodiments, L^{j} is selected from

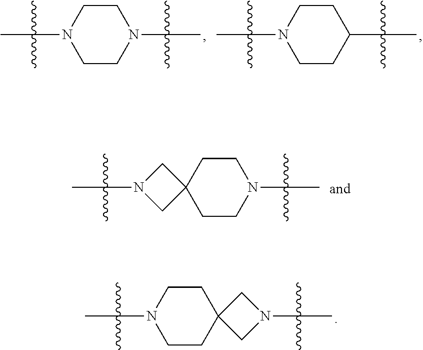 [SMILES: *N1CCN(*)CC1 ; *C1CCN(*)CC1 ; *N1CCC2(CC1)CN(*)C2 ; *N1CCC2(CC1)CN(*)C2]

In some embodiments, L^{j} is selected from

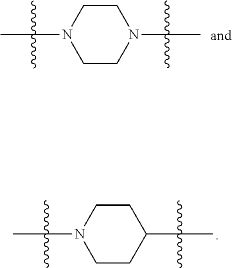 [SMILES: *N1CCN(*)CC1 ; *C1CCN(*)CC1]

In some embodiments, L^{j} is selected from

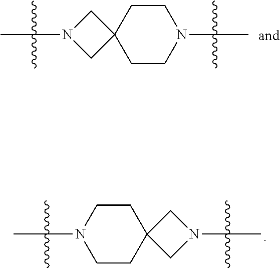 [SMILES: *N1CCC2(CC1)CN(*)C2 ; *N1CCC2(CC1)CN(*)C2]

In some embodiments, L^{j} is

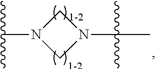

In some embodiments, L^{j} is

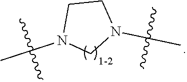

In some embodiments, L^{j} is

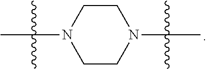 [SMILES: *N1CCN(*)CC1]

In some embodiments, L^{j} is

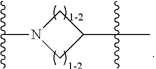

In some embodiments, L^{j} is

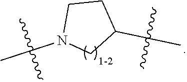

In some embodiments, L^{j} is

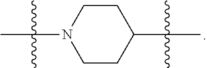 [SMILES: *C1CCN(*)CC1]

In some embodiments, L^{j} is

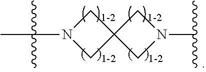

In some embodiments, L^{j} is

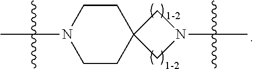

In some embodiments, L^{j} is. In some embodiments, L^{j} is

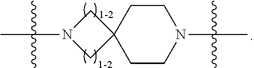

In some embodiments, L^{j} is

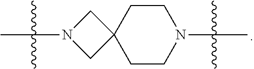 [SMILES: *N1CCC2(CC1)CN(*)C2]

In some embodiments, L^{j} is

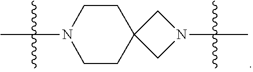 [SMILES: *N1CCC2(CC1)CN(*)C2]

In some embodiments, L^{k} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene, C₆-C₁₀ arylene, and 5-6 membered heteroarylene, wherein L^{k} is optionally substituted with one, two, three, four, or five R^{L5}. In some embodiments, L^{k} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene, C₆-C₁₀ arylene, and 5-6 membered heteroarylene, wherein L^{k} is unsubstituted.

In some embodiments, L^{k} is selected from monocyclic C₄-C₁₀ cycloalkylene, fused bicyclic C₄-C₁₀ cycloalkylene, bridged bicyclic C₆-C₁₀ cycloalkylene, or spiro-fused bicyclic C₄-C₁₂ cycloalkylene. In some embodiments, L^{k} is selected from monocyclic 4-10 membered heterocycloalkylene, fused bicyclic 4-10 membered heterocycloalkylene, bridged bicyclic 6-10 membered heterocycloalkylene, spiro-fused 4-12 membered heterocycloalkylene. In some embodiments, L^{k} is selected from C₆-C₁₀ arylene and 5-6 membered heteroarylene.

In some embodiments, L^{k} is selected from monocyclic 4-10 membered heterocycloalkylene. In some embodiments, L^{k} is selected from fused bicyclic 4-10 membered heterocycloalkylene. In some embodiments, L^{k} is selected from bridged bicyclic 6-10 membered heterocycloalkylene. In some embodiments, L^{k} is selected from spiro-fused bicyclic 4-12 membered heterocycloalkylene.

In some embodiments, L^{k} is monocyclic 4- to 10-membered heterocycloalkylene. In some embodiments, L^{k} is monocyclic 4- to 7-membered heterocycloalkylene. In some embodiments, L^{k} is monocyclic 5- or 6-membered heterocycloalkylene.

In some embodiments, L^{k} is monocyclic 4-membered heterocycloalkylene. In some embodiments, L^{k} is monocyclic 5-membered heterocycloalkylene. In some embodiments, L^{k} is monocyclic 6-membered heterocycloalkylene. In some embodiments, L^{k} is monocyclic 7-membered heterocycloalkylene. In some embodiments, L^{k} is monocyclic 8-membered heterocycloalkylene. In some embodiments, L^{k} is monocyclic 9-membered embodiments, L^{k} is monocyclic 10-membered heterocycloalkylene. In some heterocycloalkylene.

In some embodiments, L^{k} is fused bicyclic 6- to 10-membered heterocycloalkylene. In some embodiments, L^{k} is fused bicyclic 8- to 10-membered heterocycloalkylene. In some embodiments, L^{k} is fused bicyclic 5- or 6-membered heterocycloalkylene.

In some embodiments, L^{k} is fused bicyclic 4-membered heterocycloalkylene. In some embodiments, L^{k} is fused bicyclic 5-membered heterocycloalkylene. In some embodiments, L^{k} is fused bicyclic 6-membered heterocycloalkylene. In some embodiments, L^{k} is fused bicyclic 7-membered heterocycloalkylene. In some embodiments, L^{k} is fused bicyclic 8-membered heterocycloalkylene. In some embodiments, L^{k} is fused bicyclic 9-membered heterocycloalkylene. In some embodiments, L^{k} is fused bicyclic 10-membered heterocycloalkylene.

In some embodiments, L^{k} is bridged bicyclic 6- to 10-membered heterocycloalkylene. In some embodiments, L^{k} is bridged bicyclic 6 or 7-membered heterocycloalkylene.

In some embodiments, L^{k} is bridged bicyclic 6-membered heterocycloalkylene. In some embodiments, L^{k} is bridged bicyclic 7-membered heterocycloalkylene. In some embodiments, L^{k} is bridged bicyclic 8-membered heterocycloalkylene. In some embodiments, L^{k} is bridged bicyclic 9-membered heterocycloalkylene. In some embodiments, L^{k} is bridged bicyclic 10-membered heterocycloalkylene.

In some embodiments, L^{k} is spiro-fused bicyclic 4- to 12-membered heterocycloalkylene. In some embodiments, L^{k} is spiro-fused bicyclic 7- to 11-membered heterocycloalkylene. In some embodiments, L^{k} is spiro-fused bicyclic 7- or 8-membered heterocycloalkylene.

In some embodiments, L^{k} is spiro-fused bicyclic 4-membered heterocycloalkylene. In some embodiments, L^{k} is spiro-fused bicyclic 5-membered heterocycloalkylene. In some embodiments, L^{k} is spiro-fused bicyclic 6-membered heterocycloalkylene. In some embodiments, L^{k} is spiro-fused bicyclic 7-membered heterocycloalkylene. In some embodiments, L^{k} is spiro-fused bicyclic 8-membered heterocycloalkylene. In some embodiments, L^{k} is spiro-fused bicyclic 9-membered heterocycloalkylene. In some embodiments, L^{k} is spiro-fused bicyclic 10-membered heterocycloalkylene. In some embodiments, L^{k} is spiro-fused bicyclic 11-membered heterocycloalkylene. In some embodiments, L^{k} is spiro-fused bicyclic 12-membered heterocycloalkylene.

In some embodiments, L^{k} is selected from

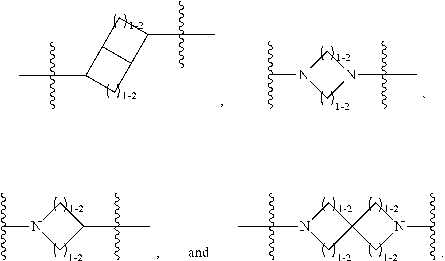

In some embodiments, L^{k} is selected from

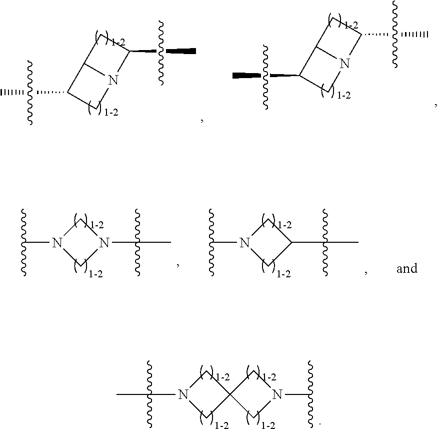

In some embodiments, L^{k} is selected from

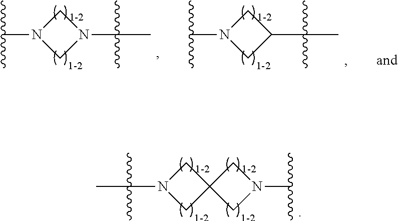

In some embodiments, L^{k} is selected from

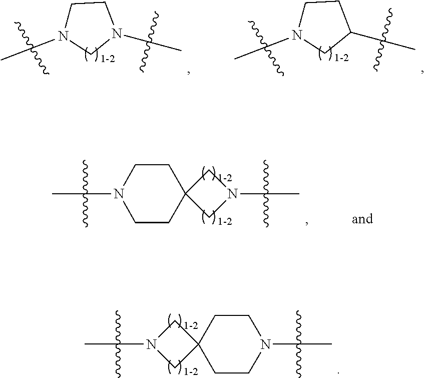

In some embodiments, L^{k} is selected from

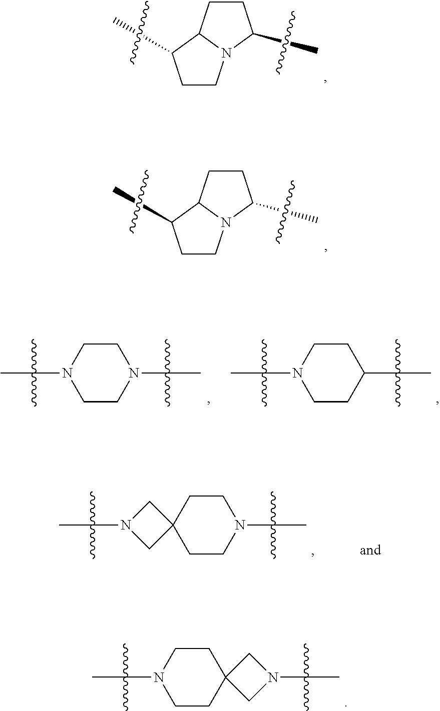

In some embodiments, L^{k} is selected from

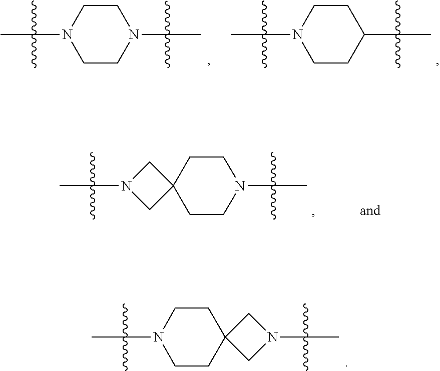 [SMILES: *N1CCN(*)CC1 ; *C1CCN(*)CC1 ; *N1CCC2(CC1)CN(*)C2 ; *N1CCC2(CC1)CN(*)C2]

In some embodiments, L^{k} is selected from

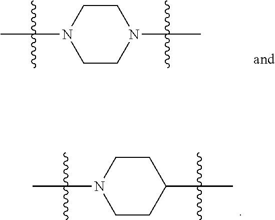 [SMILES: *N1CCN(*)CC1 ; *C1CCN(*)CC1]

In some embodiments, L^{k} is selected from

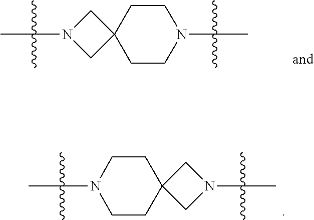 [SMILES: *N1CCC2(CC1)CN(*)C2 ; *N1CCC2(CC1)CN(*)C2]

In some embodiments, L^{k} is

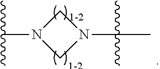

In some embodiments, L^{k} is

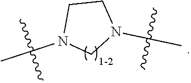

In some embodiments, L^{k} is

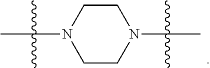 [SMILES: *N1CCN(*)CC1]

In some embodiments, L^{k} is

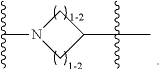

In some embodiments, L^{k} is

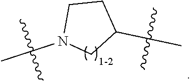

In some embodiments, L^{k} is

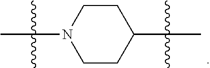 [SMILES: *C1CCN(*)CC1]

In some embodiments, L^{k} is

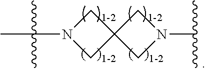

In some embodiments, L^{k} is

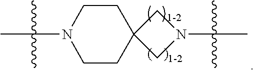

In some embodiments, L^{k} is. In some embodiments, L^{k} is

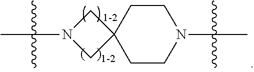

In some embodiments, L^{k} is

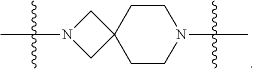 [SMILES: *N1CCC2(CC1)CN(*)C2]

In some embodiments, L^{k} is

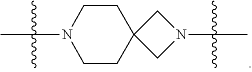 [SMILES: *N1CCC2(CC1)CN(*)C2]

In some embodiments, L^{k} is

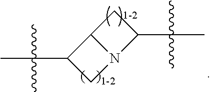

In some embodiments, L^{k} is

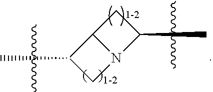

In some embodiments, L^{k} is

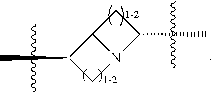

In some embodiments, L^{k} is

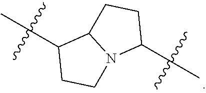 [SMILES: *C1CCN2C(*)CCC12]

In some embodiments, L^{k} is

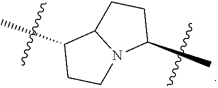

In some embodiments, L^{k} is

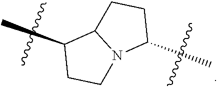

In some embodiments, (L-If′) contains no L^{l}. In some embodiments, (L-If′) contains one L^{l}. In some embodiments, (L-If′) contains two L^{l}.

In some embodiments, (L-If′) contains one L^{l} selected from -A^{L}-,

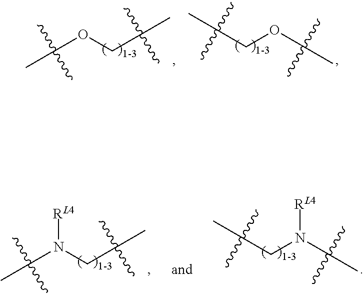

In some embodiments, (L-If′) contains one L^{l} selected from -A^{L}-,

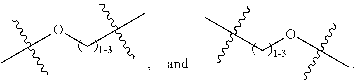

In some embodiments, (L-If′) contains one L^{l} selected from

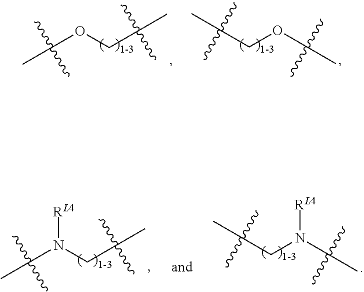

In some embodiments, (L-If′) contains one L^{l} selected from

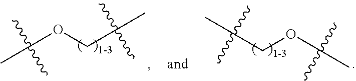

In some embodiments, (L-If′) contains one L^{l}

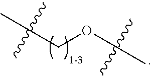

In some embodiments, (L-If′) contains one L^{l}

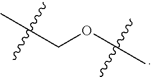 [SMILES: *CO*]

In some embodiments, (L-If′) contains one L^{l}

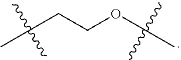 [SMILES: *CCO*]

In some embodiments, (L-If′) contains one L^{l}

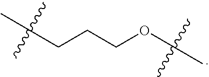 [SMILES: *CCCO*]

In some embodiments, (L-If′) contains two L^{l}, wherein both L^{l} are selected from -A^{L}-,

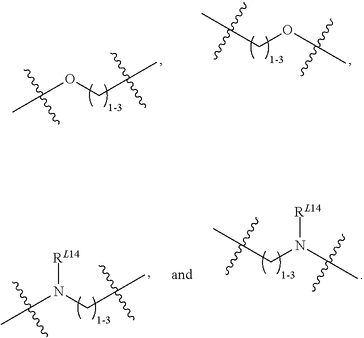

In some embodiments, (L-If′) contains two L^{l}, wherein one L^{l} is selected from -A^{L}-,

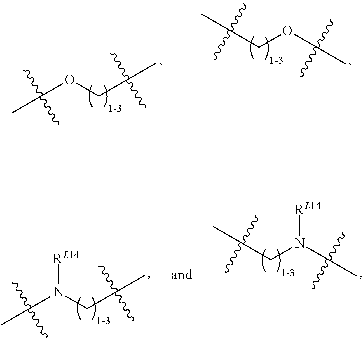

and the other L^{l} is selected from -A^{L}-,

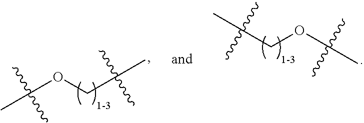

In some embodiments, (L-If′) contains two L^{l}, wherein one L^{l} is selected from -A^{L}-,

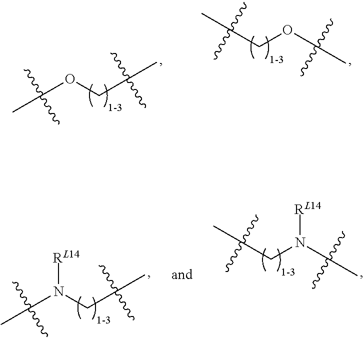

and the other L^{l} is selected from

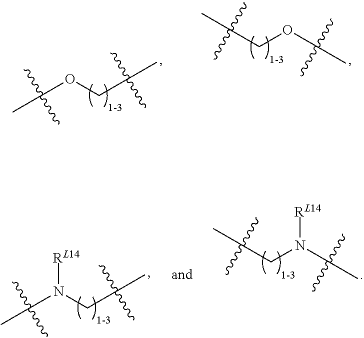

In some embodiments, contains two L^{l}, wherein one L^{l} is selected from -A^{L}-,

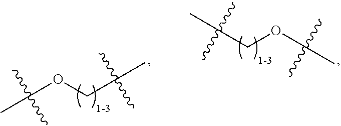

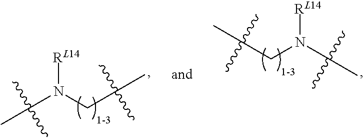

and the other L^{l} is selected from

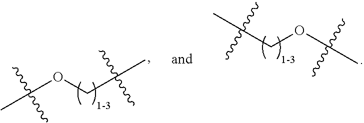

In some embodiments, contains two L^{l}, wherein one L^{l} is selected from -A^{L}-,

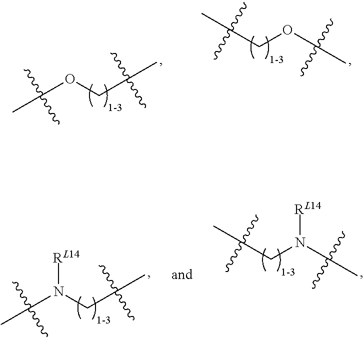

and the other L^{l} is

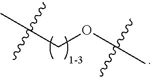

In some embodiments, contains two L^{l}, wherein one L^{l} is selected from -A^{L}-,

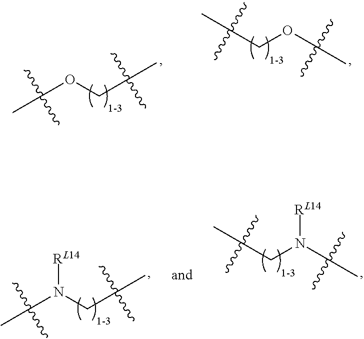

and the other L^{l} is

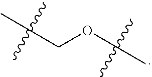 [SMILES: *CO*]

In some embodiments, contains two L^{l}, wherein one L^{l} is selected from -A^{L}-,

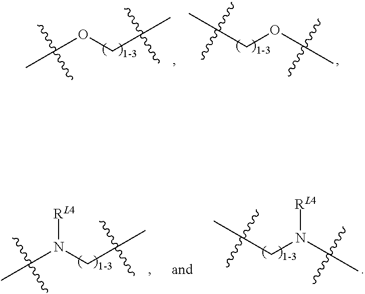

and the other L^{l} is

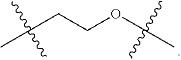 [SMILES: *CCO*]

In some embodiments, contains two L^{l}, wherein one L^{l} is selected from -A^{L}-,

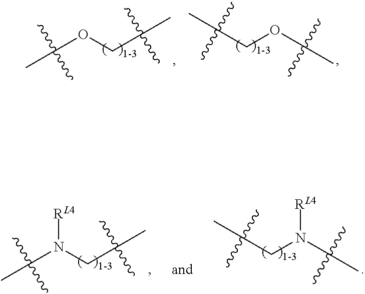

and the other L^{l} is

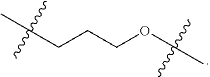 [SMILES: *CCCO*]

In some embodiments, (L-If′) contains two L^{l}, wherein one L^{l} is O, and the other L^{l} is selected from -A^{L}-,

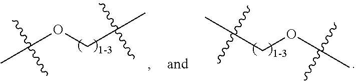

In some embodiments, (L-If′) contains two L^{l}, wherein one L^{l} is O, and the other L^{l} is selected from

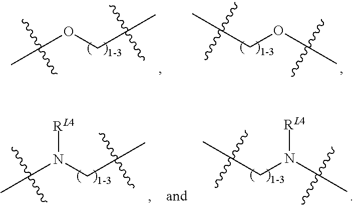

In some embodiments, contains two L^{l}, wherein one L^{l} is selected from -A^{L}-,

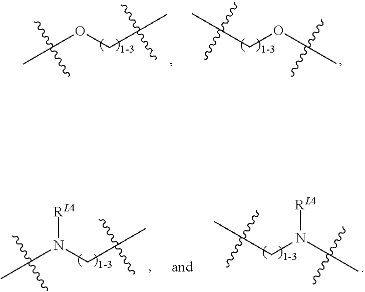

and the other L^{l} is selected from

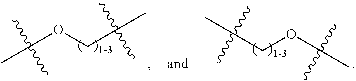

In some embodiments, contains two L^{l}, wherein one L^{l} is O, and the other L^{l} is

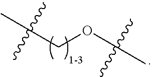

In some embodiments, contains two L^{l}, wherein one L^{l} is O, and the other L^{l} is

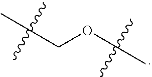 [SMILES: *CO*]

In some embodiments, contains two L^{l}, wherein one L^{l} is O, and the other L^{l} is

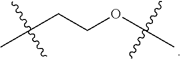 [SMILES: *CCO*]

In some embodiments, contains two L^{l}, wherein one L^{l} is O, and the other L^{l} is

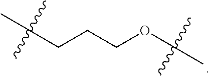 [SMILES: *CCCO*]

In some embodiments, LNK has a structure selected from (LNK-1), (LNK-2), (LNK-3), (LNK-4), (LNK-5), (LNK-6), (LNK-7), (LNK-8), (LNK-9), and (LNK-10):

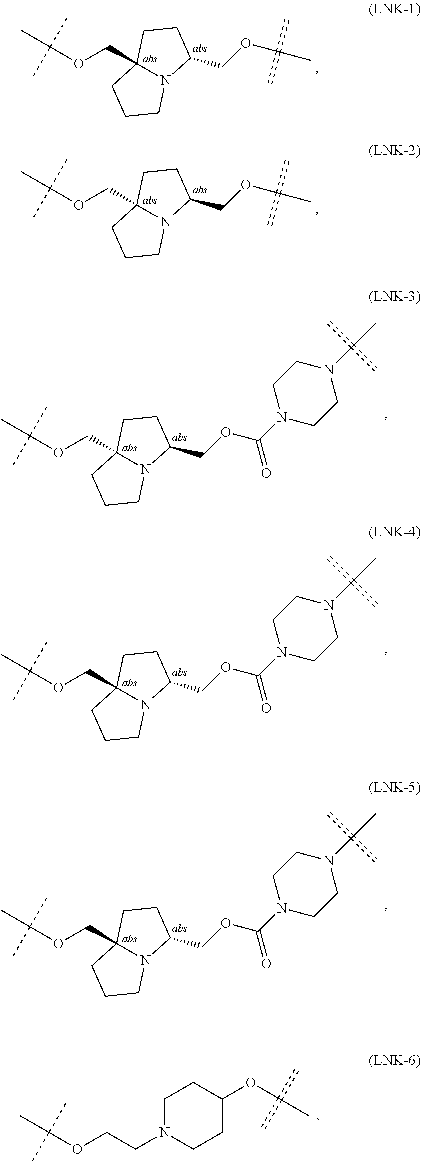

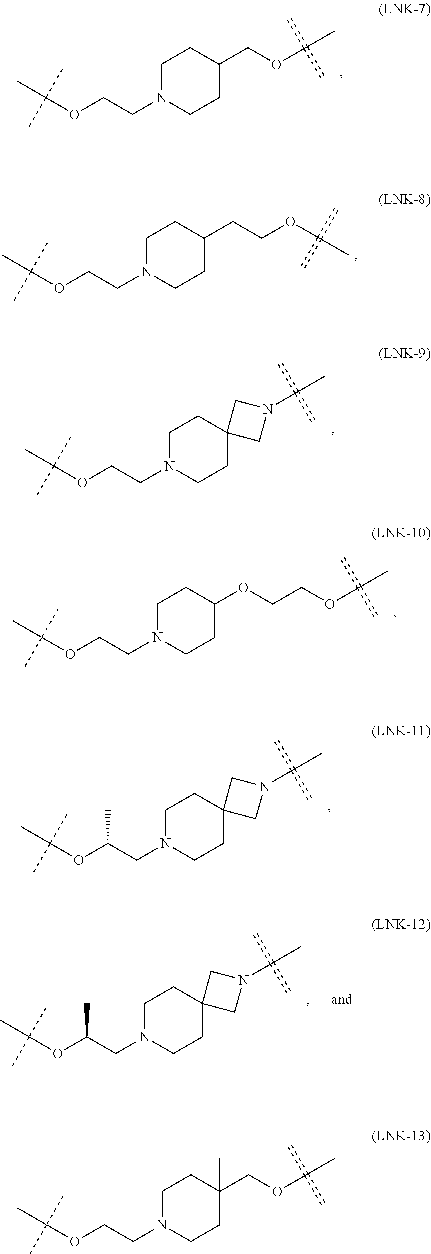

In some embodiments, LNK has the structure of (LNK-1), (LNK-2), (LNK-3), (LNK-4), or (LNK-5). In some embodiments, LNK has the structure of (LNK-6), (LNK-7), (LNK-8), (LNK-9), (LNK-10), (LNK-11), (LNK-12), or (LNK-13). In some embodiments, LNK has the structure of (LNK-9), (LNK-11), or (LNK-12). In some embodiments, LNK has the structure of (LNK-1). In some embodiments, LNK has the structure of (LNK-2). In some embodiments, LNK has the structure of (LNK-3). In some embodiments, LNK has the structure of (LNK-4). In some embodiments, LNK has the structure of (LNK-5). In some embodiments, LNK has the structure of (LNK-6). In some embodiments, LNK has the structure of (LNK-7). In some embodiments, LNK has the structure of (LNK-8). In some embodiments, LNK has the structure of (LNK-9). In some embodiments, LNK has the structure of (LNK-10). In some embodiments, LNK has the structure of (LNK-11). In some embodiments, LNK has the structure of (LNK-12). In some embodiments, LNK has the structure of (LNK-13).

In some embodiments, VLM is a Von-Hippel-Lindau (VHL) E3 ubiquitin ligase binding moiety having a structure VLM-I:

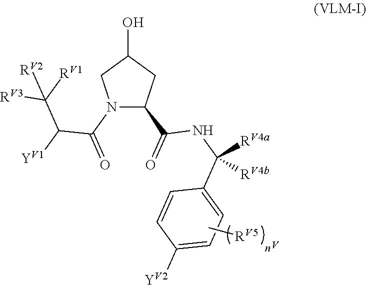

wherein:
Y^{V1} is

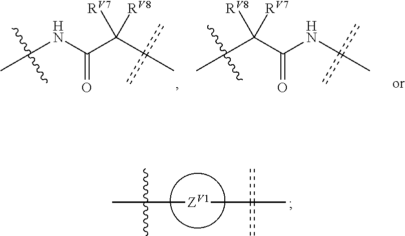

Y^{V2} is CN or

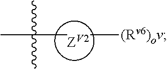

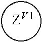

is phenylene or 5- to 6-membered heteroarylene;

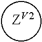

is 5-membered heteroaryl with one or two heteroatoms independently selected from N, S, and O;
R^{V1}, R^{V2}, and R^{V3} are each independently selected from H, C₁-C₆ alkyl, and C₁-C₆ haloalkyl; or, alternatively
R^{V1} and R^{V2}, together with the carbon to which they are bonded, form C₃-C₁₀ cycloalkyl or 5- to 6-membered heterocycle; and R^{V3} is selected from H, C₁-C₆ alkyl, and C₁-C₆ haloalkyl
R^{V4a} and R^{V4b} are each independently selected from H, C₁-C₆ alkyl, and C₁-C₆ haloalkyl;
each R^{V5} and R^{V6} is independently selected from H and C₁-C₆ alkyl;
R^{V7} and R^{V8} are each independently selected from H, C₁-C₆ alkyl, and C₁-C₆ haloalkyl; or, alternatively,
R^{V7} and R^{V8}, together with the atom to the carbon to which they are bonded, form C₃-C₁₀ cycloalkyl or 5- to 6-membered heterocycle;
   wherein [symbol] represents the attachment point between VLM and LNK,
n^{V} is 0, 1, 2, 3, or 4; and
o^{V} is 0, 1, 2, or 3.

In some embodiments, VLM is a Von-Hippel-Lindau (VHL) E3 ubiquitin ligase binding moiety having a structure VLM-I′:

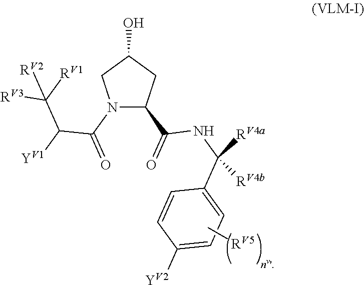

In some embodiments, VLM is a Von-Hippel-Lindau (VHL) E3 ubiquitin ligase binding moiety having a structure VLM-I″:

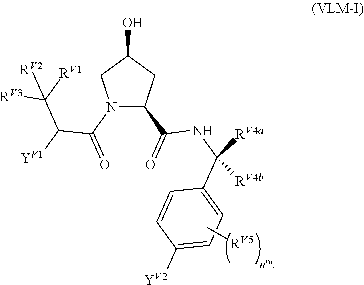

In some embodiments, VLM has the structure of formula (VLM-Ia), (VLM-Ib), (VLM-Ic), or (VLM-Id):

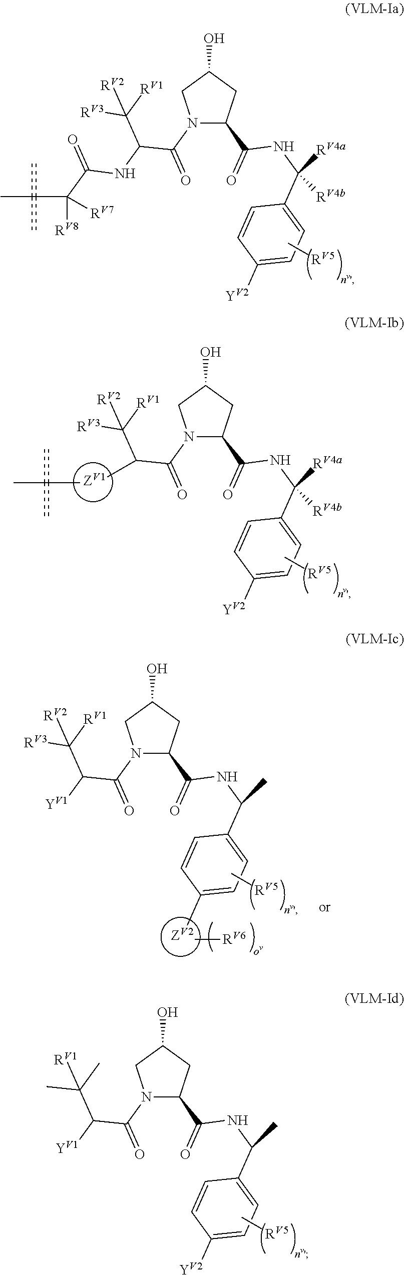

wherein [symbol] Y^{V1}, Y^{V2}, Z^{V1}, Z^{V2}, R^{V1}, R^{V2}, R^{V3}, R^{V4a}, R^{V4b} R^{V5}, R^{V6}, R^{V7}, R^{V8}, n^{V}, and o^{V} are as disclosed herein.

In some embodiments, Y^{V1} is

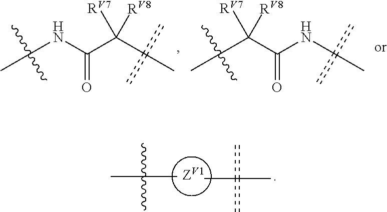

In some embodiments, Y^{V1} is

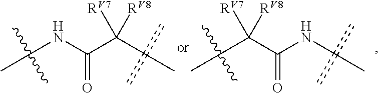

In some embodiments, Y^{V1} is

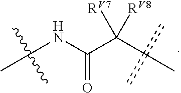

In some embodiments, Y^{V1} is

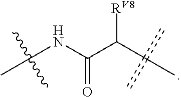

In some embodiments, Y^{V1} is

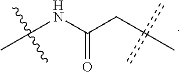

In some embodiments, Y^{V1} is

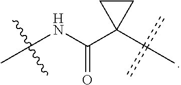

In some embodiments, Y^{V1} is

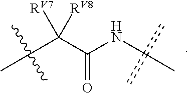

In some embodiments, Y^{V1} is

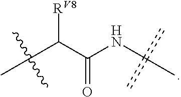

In some embodiments, Y^{V1} is

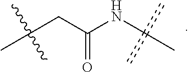

In some embodiments, Y^{V1} is

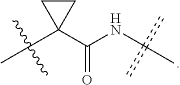

In some embodiments, Y^{V1} is

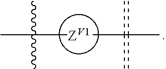

In some embodiments, Z^{V1} is phenylene. In some embodiments, Z^{V1} is 5- to 6-membered heteroarylene. In some embodiments, Z^{V1} is 5-membered heteroarylene. In some embodiments, Z^{V1} is 6-membered heteroarylene. In some embodiments, Z^{V1} is selected from oxazolylene, isoxazolylene, thiazolylene, and isothiazolylene. In some embodiments Z^{V1} is selected from oxazolylene and isoxazolylene. In some embodiments Z^{V1} is selected from thiazolylene and isothiazolylene. In some embodiments, Z^{V1} is oxazolylene. In some embodiments, Z^{V1} is isoxazolylene. In some embodiments, Z^{V1} is thiazolylene. In some embodiments, Z^{V1} is isothiazolylene.

In some embodiments, Z^{V1} is selected from

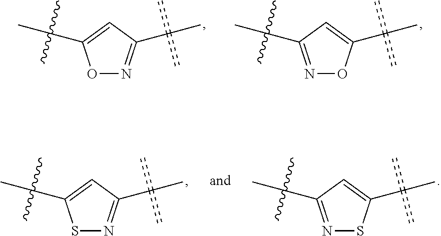

In some embodiments, Z^{V1} is selected from

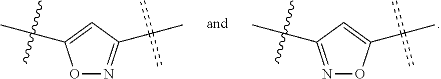

In some embodiments, Z^{V1} is selected from

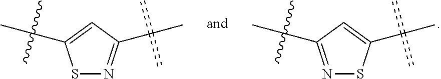

In some embodiments, Z^{V1} is

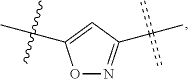

In some embodiments, Z^{V1} is

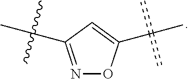

In some embodiments, Z^{V1} is

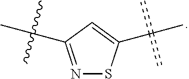

In some embodiments, Z^{V1} is

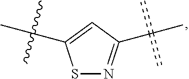

In some embodiments Y^{V2} is CN or

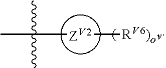

In some embodiments Y^{V2} is CN. In some embodiments, Y^{V2} is

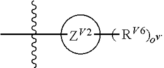

In some embodiments, Z^{V2} is 5-membered heteroaryl with one or two heteroatoms independently selected from N, S, and O. In some embodiments, Z^{V2} is 5-membered heteroaryl with one heteroatom selected from N, S, and O. In some embodiments, Z^{V2} is 5-membered heteroaryl with two heteroatoms independently selected from N, S, and O. In some embodiments, Z^{V2} is 5-membered heteroaryl with two heteroatoms independently selected from N and O. In some embodiments, Z^{V2} is 5-membered heteroaryl with two heteroatoms independently selected from N and S. In some embodiments, Z^{V2} is selected from pyrazolyl, imidazolyl, oxazolyl, isoxazolyl, thiazolyl, and isothiazolyl. In some embodiments, Z^{V2} is selected from pyrazolyl and imidazolyl. In some embodiments, Z^{V2} is selected from oxazolyl and isoxazolyl. In some embodiments, Z^{V2} is selected from thiazolyl and isothiazolyl. In some embodiments, Z^{V2} is pyrazolyl. In some embodiments, Z^{V2} is imidazolyl. In some embodiments, Z^{V2} is oxazolyl. In some embodiments, Z^{V2} is isoxazolyl. In some embodiments, Z^{V2} is thiazolyl. In some embodiments, Z^{V2} is isothiazolyl.

In some embodiments, Y^{V2} is

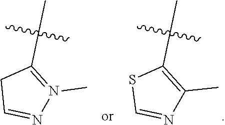

In some embodiments, Y^{V2} is

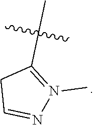

In some embodiments, Y^{V2} is

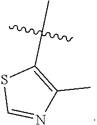 [SMILES: *c1scnc1C]

In some embodiments, o^{V} is 0, 1, 2, or 3. In some embodiments, o^{V} is 1, 2, or 3. In some embodiments, o^{V} is 0 or 1. In some embodiments, o^{V} is 0. In some embodiments, o^{V} is 1. In some embodiments, o^{V} is 2. In some embodiments, o^{V} is 3.

In some embodiments, each R^{V6} is independently selected from H and C₁-C₆ alkyl. In some embodiments, each R^{V6} is independently C₁-C₆ alkyl.

In some embodiments, o^{V} is 1 and R^{V6} is C₁-C₆ alkyl. In some embodiments, o^{V} is 1 and R^{V6} is methyl. In some embodiments, o^{V} is 1 and R^{V6} is ethyl. In some embodiments, o^{V} is 1 and R^{V6} is propyl. In some embodiments, o^{V} is 1 and R^{V6} is n-propyl. In some embodiments, o^{V} is 1 and R^{V6} is isopropyl. In some embodiments, o^{V} is 1 and R^{V6} is butyl. In some embodiments, o^{V} is 1 and R^{V6} is n-butyl. In some embodiments, o^{V} is 1 and R^{V6} is isobutyl. In some embodiments, o^{V} is 1 and R^{V6} is sec-butyl. In some embodiments, o^{V} is 1 and R^{V6} is tert-butyl. In some embodiments, o^{V} is 1 and R^{V6} is pentyl. In some embodiments, o^{V} is 1 and R^{V6} is hexyl.

In some embodiments, R^{V1}, R^{V2}, and R^{V3} are each independently selected from H, C₁-C₆ alkyl, and C₁-C₆ haloalkyl.

In some embodiments, R^{V1} is selected from H, C₁-C₆ alkyl, and C₁-C₆ haloalkyl. In some embodiments, R^{V1} is selected from C₁-C₆ alkyl and C₁-C₆ haloalkyl. In some embodiments, R^{V1} is selected from H and C₁-C₆ alkyl. In some embodiments, R^{V1} is H.

In some embodiments, R^{V1} is C₁-C₆ alkyl.

In some embodiments, R^{V1} is methyl. In some embodiments, R^{V1} is ethyl. In some embodiments, R^{V1} is propyl. In some embodiments, R^{V1} is n-propyl. In some embodiments, R^{V1} is isopropyl. In some embodiments, R^{V1} is butyl. In some embodiments, R^{V1} is n-butyl. In some embodiments, R^{V1} is isobutyl. In some embodiments, R^{V1} is sec-butyl. In some embodiments, R^{V1} is tert-butyl. In some embodiments, R^{V1} is pentyl. In some embodiments, R^{V1} is hexyl.

In some embodiments, R^{V1} is C₁-C₆ haloalkyl.

In some embodiments, R^{V1} is C₁ haloalkyl. In some embodiments, R^{V1} is C₂ haloalkyl. In some embodiments, R^{V1} is C₃ haloalkyl. In some embodiments, R^{V1} is C₄ haloalkyl. In some embodiments, R^{V1} is C₅ haloalkyl. In some embodiments, R^{V1} is C₆ haloalkyl.

In some embodiments, R^{V2} is selected from H, C₁-C₆ alkyl, and C₁-C₆ haloalkyl. In some embodiments, R^{V2} is selected from C₁-C₆ alkyl and C₁-C₆ haloalkyl. In some embodiments, R^{V2} is selected from H and C₁-C₆ alkyl. In some embodiments, R^{V2} is H.

In some embodiments, R^{V2} is C₁-C₆ alkyl.

In some embodiments, R^{V2} is methyl. In some embodiments, R^{V2} is ethyl. In some embodiments, R^{V2} is propyl. In some embodiments, R^{V2} is n-propyl. In some embodiments, R^{V2} is isopropyl. In some embodiments, R^{V2} is butyl. In some embodiments, R^{V2} is n-butyl. In some embodiments, R^{V2} is isobutyl. In some embodiments, R^{V2} is sec-butyl. In some embodiments, R^{V2} is tert-butyl. In some embodiments, R^{V2} is pentyl. In some embodiments, R^{V2} is hexyl.

In some embodiments, R^{V2} is C₁-C₆ haloalkyl.

In some embodiments, R^{V2} is C₁ haloalkyl. In some embodiments, R^{V2} is C₂ haloalkyl. In some embodiments, R^{V2} is C₃ haloalkyl. In some embodiments, R^{V2} is C₄ haloalkyl. In some embodiments, R^{V2} is C₅ haloalkyl. In some embodiments, R^{V2} is C₆ haloalkyl.

In some embodiments, R^{V3} is selected from H, C₁-C₆ alkyl, and C₁-C₆ haloalkyl. In some embodiments, R^{V3} is selected from C₁-C₆ alkyl and C₁-C₆ haloalkyl. In some embodiments, R^{V3} is selected from H and C₁-C₆ alkyl. In some embodiments, R^{V3} is H.

In some embodiments, R^{V3} is C₁-C₆ alkyl.

In some embodiments, R^{V3} is methyl. In some embodiments, R^{V3} is ethyl. In some embodiments, R^{V3} is propyl. In some embodiments, R^{V3} is n-propyl. In some embodiments, R^{V3} is isopropyl. In some embodiments, R^{V3} is butyl. In some embodiments, R^{V3} is n-butyl. In some embodiments, R^{V3} is isobutyl. In some embodiments, R^{V3} is sec-butyl. In some embodiments, R^{V3} is tert-butyl. In some embodiments, R^{V3} is pentyl. In some embodiments, R^{V3} is hexyl.

In some embodiments, R^{V3} is C₁-C₆ haloalkyl.

In some embodiments, R^{V3} is C₁ haloalkyl. In some embodiments, R^{V3} is C₂ haloalkyl. In some embodiments, R^{V3} is C₃ haloalkyl. In some embodiments, R^{V3} is C₄ haloalkyl. In some embodiments, R^{V3} is C₅ haloalkyl. In some embodiments, R^{V3} is C₆ haloalkyl.

In some embodiments, R^{V1} and R^{V2}, together with the carbon to which they are bonded, form C₃-C₁₀ cycloalkyl or 5- to 6-membered heterocycle; and R^{V3} is selected from H, C₁-C₆ alkyl, and C₁-C₆ haloalkyl. In some embodiments, R^{V1} and R^{V2}, together with the carbon to which they are bonded, form C₃-C₁₀ cycloalkyl or 5- to 6-membered heterocycle; and R^{V3} is selected from C₁-C₆ alkyl and C₁-C₆ haloalkyl. In some embodiments, R^{V1} and R^{V2}, together with the carbon to which they are bonded, form C₃-C₁₀ cycloalkyl or 5- to 6-membered heterocycle; and R^{V3} is selected from H and C₁-C₆ alkyl. In some embodiments, R^{V1} and R^{V2}, together with the carbon to which they are bonded, form C₃-C₁₀ cycloalkyl or 5- to 6-membered heterocycle; and R^{V3} is H.

In some embodiments, R^{V1} and R^{V2}, together with the carbon to which they are bonded, form C₃-C₁₀ cycloalkyl or 5- to 6-membered heterocycle.

In some embodiments, R^{V1} and R^{V2}, together with the carbon to which they are bonded, form C₃-C₁₀ cycloalkyl. In some embodiments, R^{V1} and R^{V2}, together with the carbon to which they are bonded, form cyclopropyl. In some embodiments, R^{V1} and R^{V2}, together with the carbon to which they are bonded, form cyclobutyl. In some embodiments, R^{V1} and R^{V2}, together with the carbon to which they are bonded, form cyclopentyl. In some embodiments, R^{V1} and R^{V2}, together with the carbon to which they are bonded, form cyclohexyl. In some embodiments, R^{V1} and R^{V2}, together with the carbon to which they are bonded, form cycloheptyl. In some embodiments, R^{V1} and R^{V2}, together with the carbon to which they are bonded, form cyclooctyl. In some embodiments, R^{V1} and R^{V2}, together with the carbon to which they are bonded, form cyclononyl. In some embodiments, R^{V1} and R^{V2}, together with the carbon to which they are bonded, form cyclodecyl.

In some embodiments, R^{V1} and R^{V2}, together with the carbon to which they are bonded, form 5- to 6-membered heterocycle. In some embodiments, R^{V1} and R^{V2}, together with the carbon to which they are bonded, form 5-membered heterocycle. In some embodiments, R^{V1} and R^{V2}, together with the carbon to which they are bonded, form 6-membered heterocycle.

In some embodiments, R^{V4a} and R^{V4b} are each independently selected from H, C₁-C₆ alkyl, and C₁-C₆ haloalkyl.

In some embodiments, R^{V4a} is selected from H, C₁-C₆ alkyl, and C₁-C₆ haloalkyl. In some embodiments, R^{V4a} is selected from C₁-C₆ alkyl and C₁-C₆ haloalkyl. In some embodiments, R^{V4a} is selected from H and C₁-C₆ alkyl. In some embodiments, R^{V4a} is H.

In some embodiments, R^{V4a} is C₁-C₆ alkyl.

In some embodiments, R^{V4a} is methyl. In some embodiments, R^{V4a} is ethyl. In some embodiments, R^{V4a} is propyl. In some embodiments, R^{V4a} is n-propyl. In some embodiments, R^{V4a} is isopropyl. In some embodiments, R^{V4a} is butyl. In some embodiments, R^{V4a} is n-butyl. In some embodiments, R^{V4a} is isobutyl. In some embodiments, R^{V4a} is sec-butyl. In some embodiments, R^{V4a} is tert-butyl. In some embodiments, R^{V4a} is pentyl. In some embodiments, R^{V4a} is hexyl.

In some embodiments, R^{V4a} is C₁-C₆ haloalkyl.

In some embodiments, R^{V4a} is C₁ haloalkyl. In some embodiments, R^{V4a} is C₂ haloalkyl. In some embodiments, R^{V4a} is C₃ haloalkyl. In some embodiments, R^{V4a} is C₄ haloalkyl. In some embodiments, R^{V4a} is C₅ haloalkyl. In some embodiments, R^{V4a} is C₆ haloalkyl.

In some embodiments, R^{V4b} is selected from H, C₁-C₆ alkyl, and C₁-C₆ haloalkyl. In some embodiments, R^{V4b} is selected from C₁-C₆ alkyl and C₁-C₆ haloalkyl. In some embodiments, R^{V4b} is selected from H and C₁-C₆ alkyl. In some embodiments, R^{V4b} is H.

In some embodiments, R^{V4b} is C₁-C₆ alkyl.

In some embodiments, R^{V4b} is methyl. In some embodiments, R^{V4b} is ethyl. In some embodiments, R^{V4b} is propyl. In some embodiments, R^{V4b} is n-propyl. In some embodiments, R^{V4b} is isopropyl. In some embodiments, R^{V4b} is butyl. In some embodiments, R^{V4b} is n-butyl. In some embodiments, R^{V4b} is isobutyl. In some embodiments, R^{V4b} is sec-butyl. In some embodiments, R^{V4b} is tert-butyl. In some embodiments, R^{V4b} is pentyl. In some embodiments, R^{V4b} is hexyl. In some embodiments, R^{V4b} is C₁-C₆ haloalkyl.

In some embodiments, R^{V4b} is C₁ haloalkyl. In some embodiments, R^{V4b} is C₂ haloalkyl. In some embodiments, R^{V4b} is C₃ haloalkyl. In some embodiments, R^{V4b} is C₄ haloalkyl. In some embodiments, R^{V4b} is C₅ haloalkyl. In some embodiments, R^{V4b} is C₆ haloalkyl.

In some embodiments, R^{V4b} is H and R^{V4a} is selected from H, C₁-C₆ alkyl, and C₁-C₆ haloalkyl. In some embodiments, R^{V4b} is H and R^{V4a} is selected from C₁-C₆ alkyl and C₁-C₆ haloalkyl. In some embodiments, R^{V4b} is H and R^{V4a} is selected from H and C₁-C₆ alkyl. In some embodiments, R^{V4b} is H and R^{V4a} is H.

In some embodiments, R^{V4b} is H and R^{V4a} is C₁-C₆ alkyl.

In some embodiments, R^{V4b} is H and R^{V4a} is methyl. In some embodiments, R^{V4b} is H and R^{V4a} is ethyl. In some embodiments, R^{V4b} is H and R^{V4a} is propyl. In some embodiments, R^{V4b} is H and R^{V4a} is n-propyl. In some embodiments, R^{V4b} is H and R^{V4a} is isopropyl. In some embodiments, R^{V4b} is H and R^{V4a} is butyl. In some embodiments, R^{V4b} is H and R^{V4a} is n-butyl. In some embodiments, R^{V4b} is H and R^{V4a} is isobutyl. In some embodiments, R^{V4b} is H and R^{V4a} is sec-butyl. In some embodiments, R^{V4b} is H and R^{V4a} is tert-butyl. In some embodiments, R^{V4b} is H and R^{V4a} is pentyl. In some embodiments, R^{V4b} is H and R^{V4a} is hexyl.

In some embodiments, R^{V4b} is H and R^{V4a} is C₁-C₆ haloalkyl.

In some embodiments, R^{V4b} is H and R^{V4a} is C₁ haloalkyl. In some embodiments, R^{V4b} is H and R^{V4a} is C₂ haloalkyl. In some embodiments, R^{V4b} is H and R^{V4a} is C₃ haloalkyl. In some embodiments, R^{V4b} is H and R^{V4a} is C₄ haloalkyl. In some embodiments, R^{V4b} is H and R^{V4a} is C₅ haloalkyl. In some embodiments, R^{V4b} is H and R^{V4a} is C₆ haloalkyl.

In some embodiments, R^{V4a} is H and R^{V4b} is selected from H, C₁-C₆ alkyl, and C₁-C₆ haloalkyl. In some embodiments, R^{V4a} is H and R^{V4b} is selected from C₁-C₆ alkyl and C₁-C₆ haloalkyl. In some embodiments, R^{V4a} is H and R^{V4b} is selected from H and C₁-C₆ alkyl. In some embodiments, R^{V4a} is H and R^{V4b} is H.

In some embodiments, R^{V4a} is H and R^{V4b} is C₁-C₆ alkyl.

In some embodiments, R^{V4a} is H and R^{V4b} is methyl. In some embodiments, R^{V4a} is H and R^{V4b} is ethyl. In some embodiments, R^{V4a} is H and R^{V4b} is propyl. In some embodiments, R^{V4a} is H and R^{V4b} is n-propyl. In some embodiments, R^{V4a} is H and R^{V4b} is isopropyl. In some embodiments, R^{V4a} is H and R^{V4b} is butyl. In some embodiments, R^{V4a} is H and R^{V4b} is n-butyl. In some embodiments, R^{V4a} is H and R^{V4b} is isobutyl. In some embodiments, R^{V4a} is H and R^{V4b} is sec-butyl. In some embodiments, R^{V4a} is H and R^{V4b} is tert-butyl. In some embodiments, R^{V4a} is H and R^{V4b} is pentyl. In some embodiments, R^{V4a} is H and R^{V4b} is hexyl.

In some embodiments, R^{V4a} is H and R^{V4b} is C₁-C₆ haloalkyl.

In some embodiments, R^{V4a} is H and R^{V4b} is C₁ haloalkyl. In some embodiments, R^{V4a} is H and R^{V4b} is C₂ haloalkyl. In some embodiments, R^{V4a} is H and R^{V4b} is C₃ haloalkyl. In some embodiments, R^{V4a} is H and R^{V4b} is C₄ haloalkyl. In some embodiments, R^{V4a} is H and R^{V4b} is C₅ haloalkyl. In some embodiments, R^{V4a} is H and R^{V4b} is C₆ haloalkyl.

In some embodiments, R^{V7} is selected from H, C₁-C₆ alkyl, and C₁-C₆ haloalkyl. In some embodiments, R^{V7} is selected from C₁-C₆ alkyl and C₁-C₆ haloalkyl. In some embodiments, R^{V7} is selected from H and C₁-C₆ alkyl. In some embodiments, R^{V7} is H.

In some embodiments, R^{V7} is C₁-C₆ alkyl.

In some embodiments, R^{V7} is methyl. In some embodiments, R^{V7} is ethyl. In some embodiments, R^{V7} is propyl. In some embodiments, R^{V7} is n-propyl. In some embodiments, R^{V7} is isopropyl. In some embodiments, R^{V7} is butyl. In some embodiments, R^{V7} is n-butyl. In some embodiments, R^{V7} is isobutyl. In some embodiments, R^{V7} is sec-butyl. In some embodiments, R^{V7} is tert-butyl. In some embodiments, R^{V7} is pentyl. In some embodiments, R^{V7} is hexyl.

In some embodiments, R^{V7} is C₁-C₆ haloalkyl.

In some embodiments, R^{V7} is C₁ haloalkyl. In some embodiments, R^{V7} is C₂ haloalkyl. In some embodiments, R^{V7} is C₃ haloalkyl. In some embodiments, R^{V7} is C₄ haloalkyl. In some embodiments, R^{V7} is C₅ haloalkyl. In some embodiments, R^{V7} is C₆ haloalkyl.

In some embodiments, R^{V8} is selected from H, C₁-C₆ alkyl, and C₁-C₆ haloalkyl. In some embodiments, R^{V8} is selected from C₁-C₆ alkyl and C₁-C₆ haloalkyl. In some embodiments, R^{V8} is selected from H and C₁-C₆ alkyl. In some embodiments, R^{V8} is H.

In some embodiments, R^{V8} is C₁-C₆ alkyl.

In some embodiments, R^{V8} is methyl. In some embodiments, R^{V8} is ethyl. In some embodiments, R^{V8} is propyl. In some embodiments, R^{V8} is n-propyl. In some embodiments, R^{V8} is isopropyl. In some embodiments, R^{V8} is butyl. In some embodiments, R^{V8} is n-butyl. In some embodiments, R^{V8} is isobutyl. In some embodiments, R^{V8} is sec-butyl. In some embodiments, R^{V8} is tert-butyl. In some embodiments, R^{V8} is pentyl. In some embodiments, R^{V8} is hexyl.

In some embodiments, R^{V8} is C₁-C₆ haloalkyl.

In some embodiments, R^{V8} is C₁ haloalkyl. In some embodiments, R^{V8} is C₂ haloalkyl. In some embodiments, R^{V8} is C₃ haloalkyl. In some embodiments, R^{V8} is C₄ haloalkyl. In some embodiments, R^{V8} is C₅ haloalkyl. In some embodiments, R^{V8} is C₆ haloalkyl.

In some embodiments, R^{V7} and R^{V8}, together with the carbon to which they are bonded, form C₃-C₁₀ cycloalkyl or 5- to 6-membered heterocycle.

In some embodiments, R^{V7} and R^{V8}, together with the carbon to which they are bonded, form C₃-C₁₀ cycloalkyl. In some embodiments, R^{V7} and R^{V8}, together with the carbon to which they are bonded, form cyclopropyl. In some embodiments, R^{V7} and R^{V8} together with the carbon to which they are bonded, form cyclobutyl. In some embodiments, R^{V7} and R^{V8}, together with the carbon to which they are bonded, form cyclopentyl. In some embodiments, R^{V7} and R^{V8}, together with the carbon to which they are bonded, form cyclohexyl. In some embodiments, R^{V7} and R^{V8}, together with the carbon to which they are bonded, form cycloheptyl. In some embodiments, R^{V7} and R^{V8}, together with the carbon to which they are bonded, form cyclooctyl. In some embodiments, R^{V7} and R^{V8}, together with the carbon to which they are bonded, form cyclononyl. In some embodiments, R^{V7} and R^{V8}, together with the carbon to which they are bonded, form cyclodecyl.

In some embodiments, R^{V7} and R^{V8}, together with the carbon to which they are bonded, form 5- to 6-membered heterocycle. In some embodiments, R^{V7} and R^{V8}, together with the carbon to which they are bonded, form 5-membered heterocycle. In some embodiments, R^{V7} and R^{V8}, together with the carbon to which they are bonded, form 6-membered heterocycle.

In some embodiments, n° is 0, 1, 2, 3 or. In some embodiments, n^{V} is 1, 2, 3 or 4. In some embodiments, n is 0 or 1. In some embodiments, n^{V} is 0. In some embodiments, n^{V} is 1. In some embodiments, n° is 2. In some embodiments, n^{V} is 3. In some embodiments, n^{V} is 4.

In some embodiments, each R^{V5} is independently selected from H and C₁-C₆ alkyl. In some embodiments, each R^{V5} is independently C₁-C₆ alkyl.

In some embodiments, n is 1 and R^{V5} is C₁-C₆ alkyl. In some embodiments, n° is 1 and R^{V5} is methyl. In some embodiments, n^{V} is 1 and R^{V5} is ethyl. In some embodiments, n^{V} is 1 and R^{V5} is propyl. In some embodiments, n^{V} is 1 and R^{V5} is n-propyl. In some embodiments, n^{V} is 1 and R^{V5} is isopropyl. In some embodiments, n^{V} is 1 and R^{V5} is butyl. In some embodiments, n^{V} is 1 and R^{V5} is n-butyl. In some embodiments, n^{V} is 1 and R^{V5} is isobutyl. In some embodiments, n^{V} is 1 and R^{V5} is sec-butyl. In some embodiments, n^{V} is 1 and R^{V5} is tert-butyl. In some embodiments, n^{V} is 1 and R^{V5} is pentyl. In some embodiments, n^{V} is 1 and R^{V5} is hexyl.

In some embodiments, VLM has a structure selected from (VLM-1), (VLM-2), (VLM-3), (VLM-4), (VLM-5), (VLM-6), (VLM-7), (VLM-8), (VLM-9), and (VLM-10):

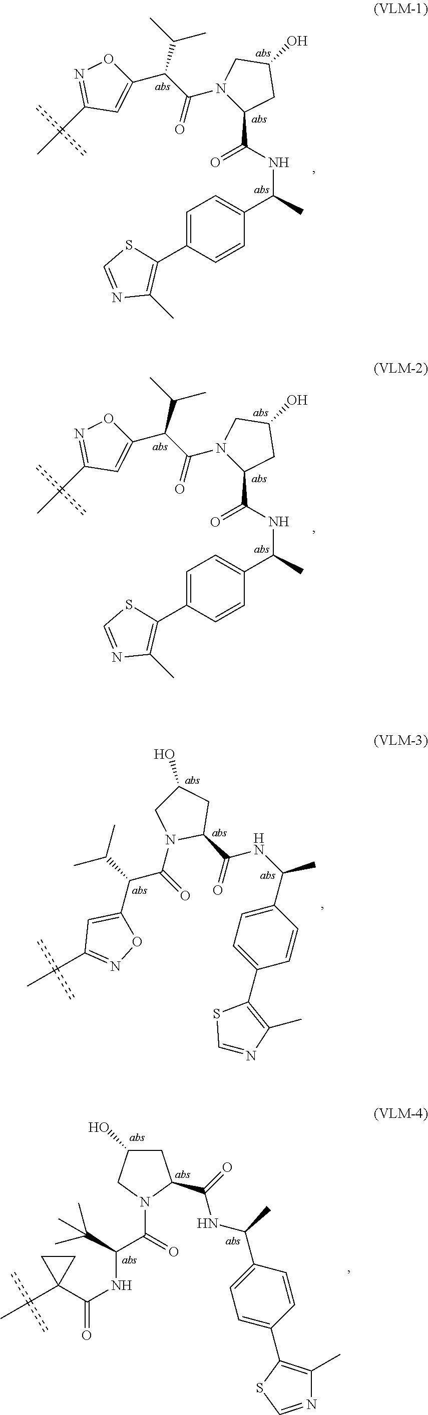

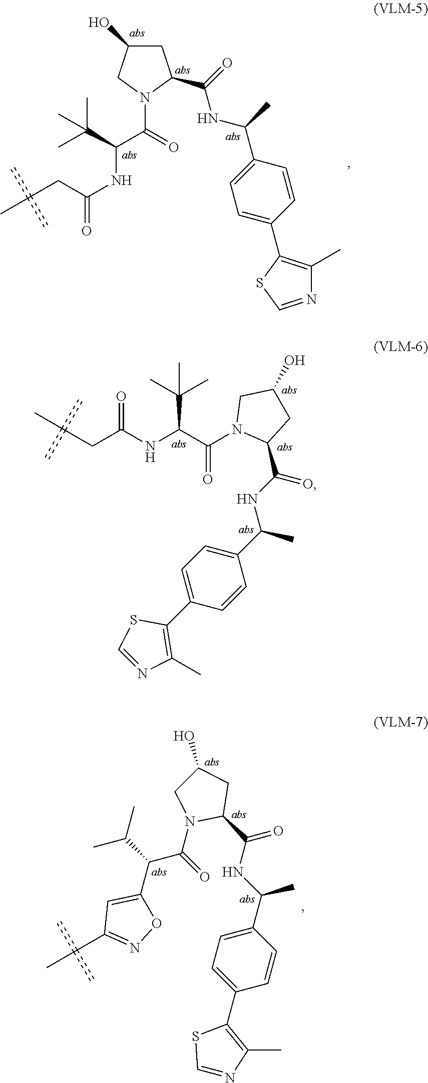

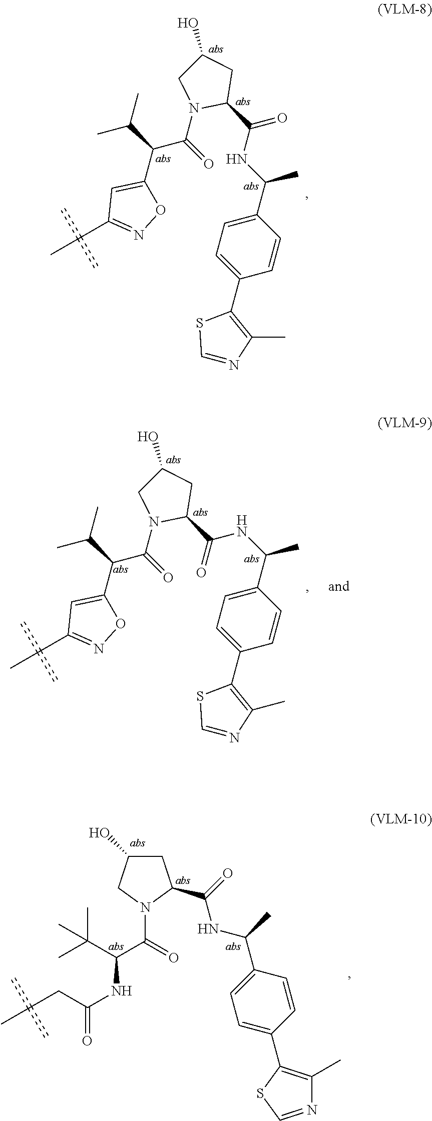

In some embodiments, VLM has the structure of (VLM-1). In some embodiments, VLM has the structure of (VLM-2). In some embodiments, VLM has the structure of (VLM-3). In some embodiments, VLM has the structure of (VLM-4). In some embodiments, VLM has the structure of (VLM-5). In some embodiments, VLM has the structure of (VLM-6). In some embodiments, VLM has the structure of (VLM-7). In some embodiments, VLM has the structure of (VLM-8). In some embodiments, VLM has the structure of (VLM-9). In some embodiments, VLM has the structure of (VLM-10).

In embodiments, the compound of Formula I has a structure according to Formula II:

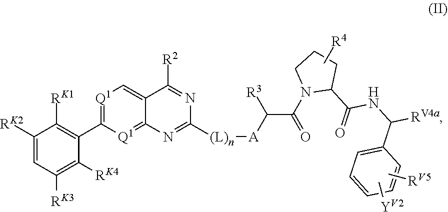

or a pharmaceutically acceptable salt thereof,

wherein,
Q¹ is CR¹ or N;
R^{K1}, R^{K2}, R^{K3}, and R^{K4} are each independently selected from H, OH, Cl, F, Br, I, C₁-C₆ alkyl, C₁-C₆ haloalkyl, O—C₁-C₆ alkyl, O—C₁-C₆ haloalkyl, C₃-C₁₀ cycloalkyl, and 3-10-membered heterocycle;
alternatively, R^{K3} and R^{K4}, together with the carbons to which they are bonded, form C₆ aryl or 5-6 membered heteroaryl, wherein aryl and heteroaryl are optionally substituted with one or two R^{K11};
each R^{K11} is independently selected from OH, CN, Cl, F, Br, I, C₁-C₆ alkyl, C₂-C₆ alkenyl, C₂-C₆ alkynyl, and C₁-C₆ haloalkyl;
R¹ is selected from H, OH, CN, Cl, F, Br, I, and C₁-C₆ alkyl;
R² is selected from H, C₁-C₆ alkyl, 4-10 membered cycloalkyl, 4-10 membered heterocycloalkyl, and C₆-C₁₀ aryl, wherein C₁-C₆ alkyl, 4-10 membered cycloalkyl, 4-10 membered heterocycloalkyl, and C₆-C₁₀ aryl are optionally substituted with OH, C(O)OH, C(O)H, or C(O)OC₁₋₆ alkyl;
R³ is selected from H, OH, CN, Cl, F, Br, I, C₁-C₆ alkyl, and C₁-C₆ haloalkyl;
R⁴ is selected from H, OH, CN, Cl, F, Br, I, and C₁-C₆ alkyl;
R^{V4a} is selected from H, C₁-C₆ alkyl, and C₁-C₆ haloalkyl;
R^{V5} is selected from H, halo, and C₁-C₆ alkyl;
Y^{V2} is CN or 5-10 membered heteroaryl with one or two heteroatoms independently selected from N, S, and O, wherein 5-10 membered heteroaryl is optionally substituted with C₁-C₆ alkyl or C₁-C₆ haloalkyl;
each L is independently selected from CH₂, NH, N(C₁-C₆ alkyl), O, C(O), 5-10 membered heteroarylene, C₂-C₆ alkylene, monocyclic C₄-C₁₀ cycloalkylene, monocyclic 4-10 membered heterocycloalkylene, fused bicyclic 4-10 membered heterocycloalkylene, and spiro-fused 5-12 membered heterocycloalkylene, wherein each heterocycloalkylene is optionally substituted with one or two instances of halo, C₁-C₆ alkyl, O—C₁-C₆ alkyl, and C₁-C₆ haloalkyl;
A is selected from C(O), NH, C(O)N(H), N(H) C(O), 6-10 membered arylene, and 5-10 membered heteroarylene; and
n is 1, 2, 3, 4, 5, 6, 7, 8, 9, 10, 11, 12, 13, 14, or 15.

In embodiments of Formula II,
Q¹ is CR¹ or N;
R^{K1}, R^{K2}, R^{K3}, and R^{K4} are each independently selected from H, OH, Cl, F, Br, I, C₁-C₆ alkyl, C₁-C₆ haloalkyl, O—C₁-C₆ alkyl, O—C₁-C₆ haloalkyl, and C₃-C₁₀ cycloalkyl;
alternatively, R^{K3} and R^{K4}, together with the carbons to which they are bonded, form C₆ aryl or 5-6-membered heteroaryl, wherein the aryl or heteroaryl is optionally substituted with one or two R^{K11};
each R^{K11} is independently selected from H, OH, CN, Cl, F, Br, I, C₁-C₆ alkyl, C₂-C₆ alkenyl, C₂-C₆ alkynyl, and C₁-C₆ haloalkyl;
R¹ is selected from H, OH, CN, Cl, F, Br, and I;
R² is selected from H, C₁-C₆ alkyl, and 4-10 membered heterocycloalkyl, wherein 4-10 membered heterocycloalkyl is optionally substituted with OH, C(O)OH, C(O)H, or C(O)OC₁₋₆ alkyl;
R³ is selected from H, C₁-C₆ alkyl, and C₁-C₆ haloalkyl;
R⁴ is selected from H, OH, CN, Cl, F, Br, and I;
R^{V4a} is selected from H, C₁-C₆ alkyl, and C₁-C₆ haloalkyl;
R^{V5} is selected from H, halo, and C₁-C₆ alkyl;
Y^{V2} is CN or 5-7 membered heteroaryl with one or two heteroatoms independently selected from N, S, and O, wherein 5-7 membered heteroaryl is optionally substituted with C₁-C₆ alkyl or C₁-C₆ haloalkyl;
each L is independently selected from CH₂, NH, N(C₁-C₆ alkyl), O, C(O), 5-10 membered heteroarylene, C₂-C₆ alkylene, monocyclic C₄-C₁₀ cycloalkylene, monocyclic 4-10 membered heterocycloalkylene, fused bicyclic 4-10 membered heterocycloalkylene, and spiro-fused 5-12 membered heterocycloalkylene, wherein each heterocycloalkylene is optionally substituted with one or two instances of halo, C₁-C₆ alkyl, O—C₁-C₆ alkyl, and C₁-C₆ haloalkyl;
A is selected from C(O)N(H), N(H) C(O), and 5-7 membered heteroarylene; and
n is 1, 2, 3, 4, 5, 6, 7, 8, 9, or 10.

In embodiments of Formula II,
Q¹ is CR¹ or N;
R^{K1}, R^{K2}, R^{K3}, and R^{K4} are each independently selected from H, OH, Cl, F, Br, I, C₁-C₆ alkyl, C₁-C₆ haloalkyl, O—C₁-C₆ alkyl, O—C₁-C₆ haloalkyl, and C₃-C₆ cycloalkyl;
alternatively, R^{K3} and R^{K4}, together with the carbons to which they are bonded, form C₆ aryl or 5-6-membered heteroaryl, wherein the aryl or heteroaryl is optionally substituted with one or two R^{K11};
each R^{K11} is independently selected from H, OH, CN, Cl, F, Br, I, C₁-C₆ alkyl, C₂-C₆ alkenyl, C₂-C₆ alkynyl, and C₁-C₆ haloalkyl;
R¹ is selected from H, OH, CN, Cl, F, Br, and I;
R² is selected from H, C₁-C₆ alkyl, and 7-8 membered heterocycloalkyl, wherein 7-8 membered heterocycloalkyl is optionally substituted with OH, C(O)OH, C(O)H, or C(O)OC₁₋₆ alkyl;
R³ is selected from H, C₁-C₆ alkyl, and C₁-C₆ haloalkyl; R⁴ is selected from H, OH, CN, Cl, F, Br, and I;
R^{V4a} is selected from H, C₁-C₆ alkyl, and C₁-C₆ haloalkyl;
R^{V5} is selected from H, halo, and C₁-C₆ alkyl;
Y^{V2} is CN or 5-membered heteroaryl with one or two heteroatoms independently selected from N, S, and O, wherein 5-membered heteroaryl is optionally substituted with C₁-C₆ alkyl or C₁-C₆ haloalkyl;
each L is independently selected from CH₂, NH, N(C₁-C₆ alkyl), O, C(O), 5-7 membered heteroarylene, C₂-C₆ alkylene, monocyclic C₄-C₁₀ cycloalkylene, monocyclic 4-10 membered heterocycloalkylene, fused bicyclic 4-10 membered heterocycloalkylene, and spiro-fused 5-12 membered heterocycloalkylene, wherein each heterocycloalkylene is optionally substituted with one or two instances of halo, C₁-C₆ alkyl, O—C₁-C₆ alkyl, and C₁-C₆ haloalkyl;
A is selected from C(O)N(H), N(H) C(O), and 5-membered heteroarylene; and
n is 1, 2, 3, 4, 5, 6, 7, 8, 9, or 10.

In embodiments, the compound of Formula I has a structure according to Formula IIa:

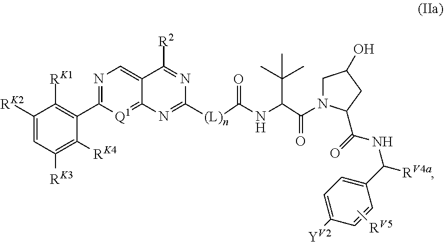

or a pharmaceutically acceptable salt thereof.

In embodiments, the compound of Formula I has a structure according to Formula IIb:

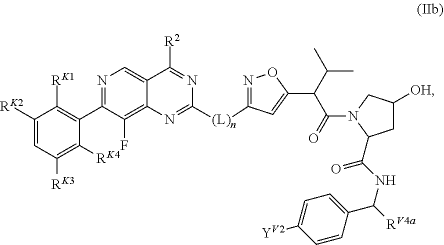

or a pharmaceutically acceptable salt thereof.

In embodiments, the compound of Formula I has a structure according to Formula IIc:

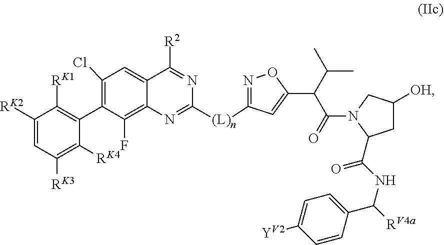

or a pharmaceutically acceptable salt thereof.

In embodiments of Formula IIa, the compound has a structure according to one of Formula IIa-i through Formula IIa-v:

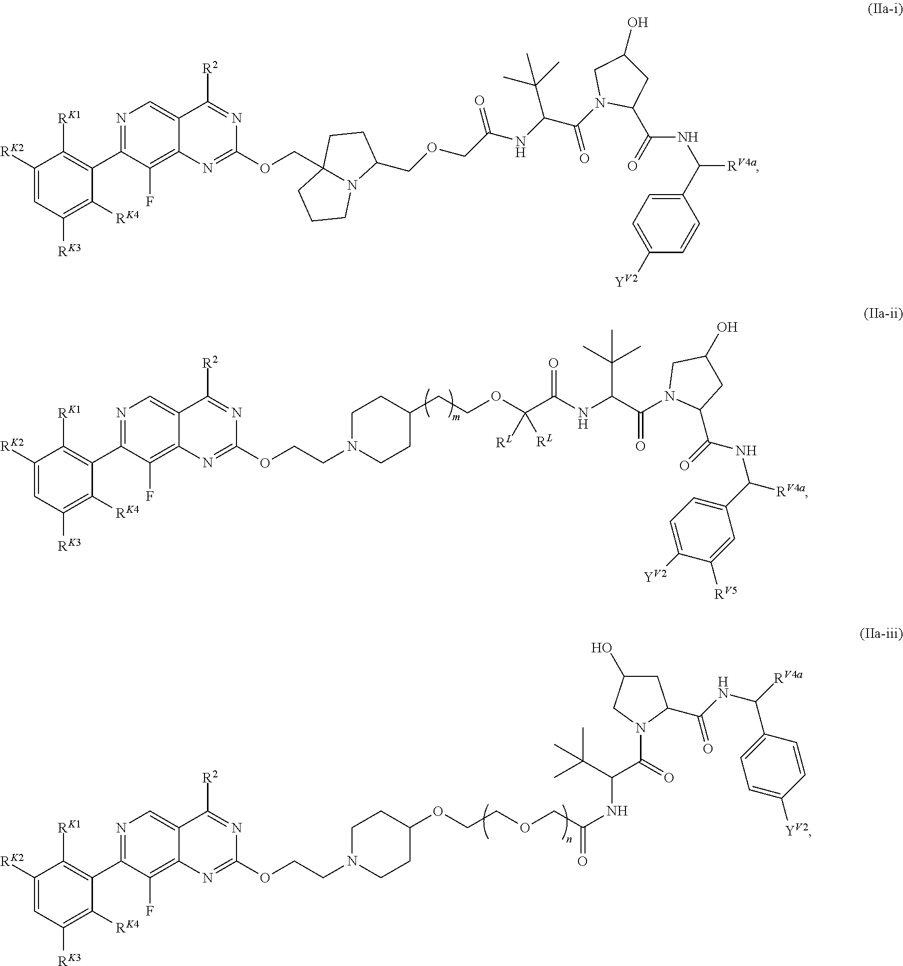

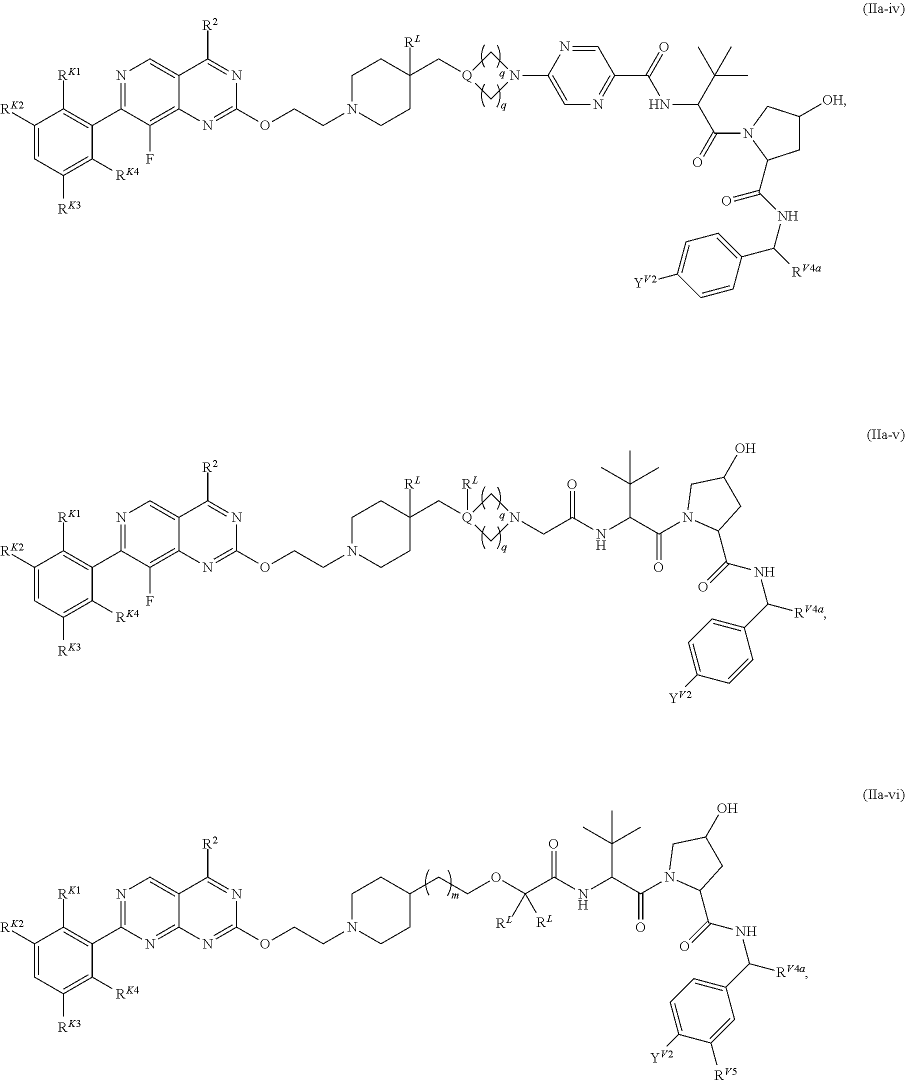

or a pharmaceutically acceptable salt thereof,

wherein,
each R^{L} is independently selected from H, halo, C₁-C₆ alkyl, C₁-C₆ haloalkyl, or O—C₁-C₆ alkyl;
alternatively, both R^{L}, together with the carbons to which they are bonded, form C₃-C₆ cycloalkyl;
Q is CR^{L} or N; and
m, n, and q are each independently 0, 1, or 2.

In embodiments of Formula IIb, the compound has a structure according to one of Formula IIb-i through Formula IIb-vi:

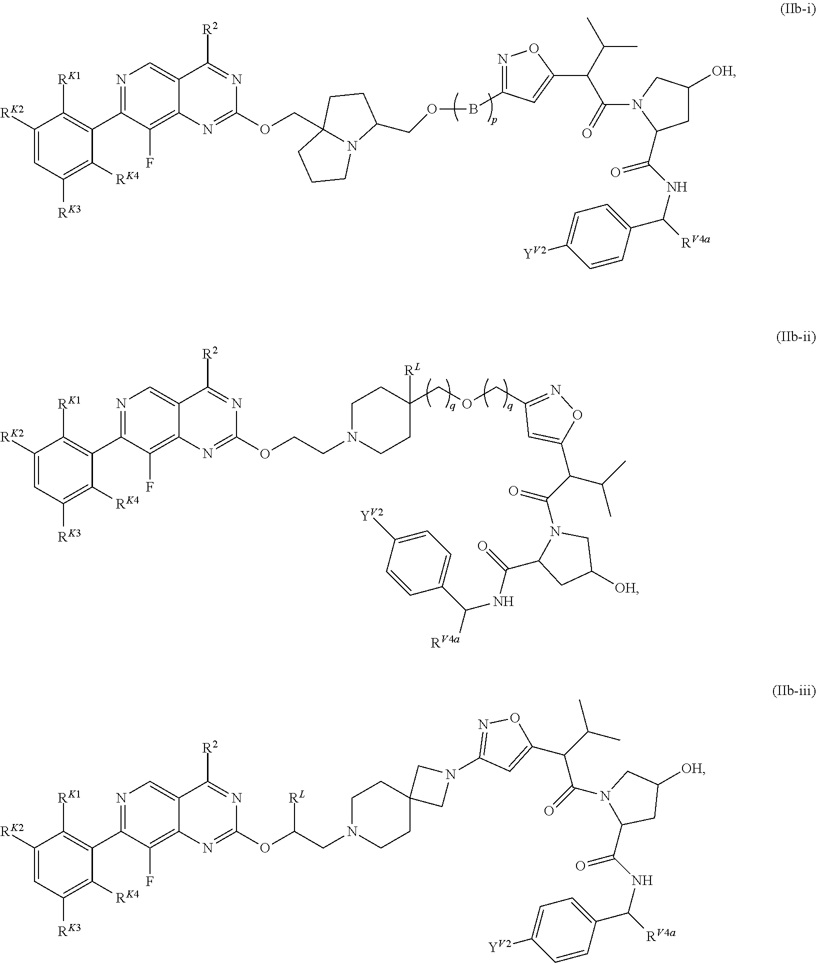

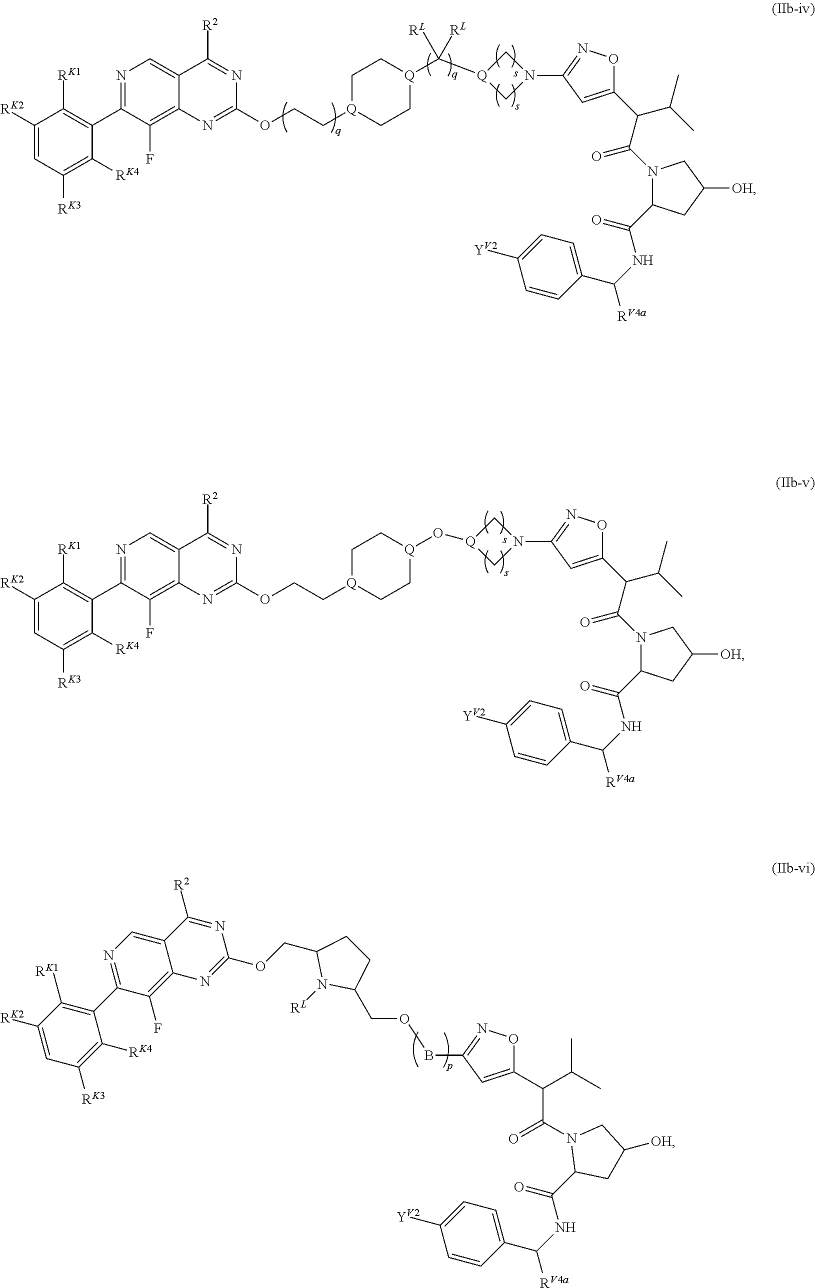

or a pharmaceutically acceptable salt thereof,

wherein,
B is selected from:

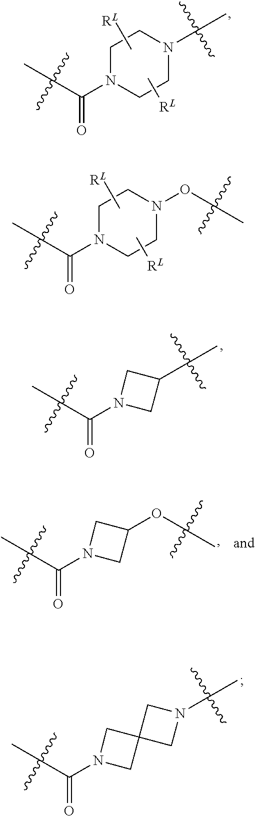

each Q is independently CR^{L} or N;
R^{L} is H, OH, halo, C₁-C₆ alkyl, C₁-C₆ haloalkyl, or O—C₁-C₆ alkyl; and
p is 0 or 1;
each q is independently 0, 1, or 2; and
each s is independently 1 or 2.

In embodiments of Formula IIc, the compound has a structure according to Formula IIc-i:

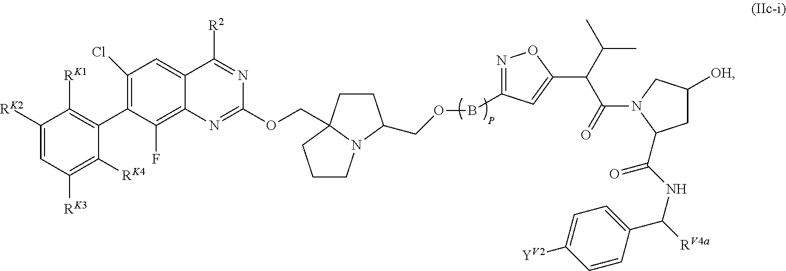

or a pharmaceutically acceptable salt thereof,

wherein,
B is selected from:

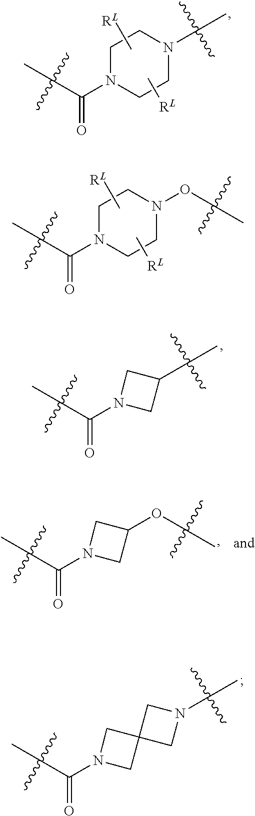

R^{L} is H, OH, halo, C₁-C₆ alkyl, C₁-C₆ haloalkyl, or O—C₁-C₆ alkyl; and
p is 0 or 1.

In embodiments, R² is selected from:

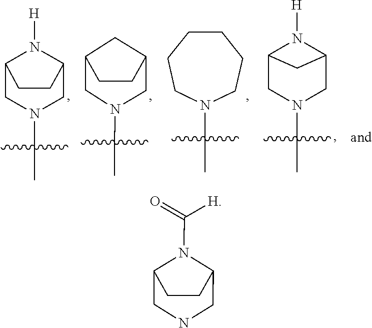

In embodiments, R^{K1}, R^{K2}, R^{K3}, and R^{K4} are each independently selected from H, OH, Cl, F, Br, I, C₁-C₆ alkyl, C₁-C₆ haloalkyl, O—C₁-C₆ haloalkyl, and C₃-C₈ cycloalkyl.

In embodiments, R^{K3} and R^{K4}, together with the carbons to which they are bonded, form C₆ aryl, wherein aryl is optionally substituted with one or two Cl, F, Br, I, C₁-C₆ alkyl, C₂-C₆ alkynyl, and C₁-C₆ haloalkyl.

In embodiments, R^{K3} and R^{K4}, together with the carbons to which they are bonded, form 5-membered heteroaryl.

In embodiments, R¹ is selected from Cl, F, Br, and I. In embodiments, R² is 7-8 membered heterocycloalkyl. In embodiments, R³ is C₁-C₆ alkyl. In embodiments, R⁴ is OH.

In embodiments, R^{V4a} is H or C₁-C₆ alkyl. In embodiments, Y^{V2} is CN or 5-membered heteroaryl with one or two heteroatoms independently selected from N, S, and O, wherein 5-membered heteroaryl is optionally substituted with C₁-C₆ alkyl or C₁-C₆ haloalkyl. In embodiments Y^{V2} is CN. In embodiments, Y^{V2} is thiazolyl optionally substituted with C₁-C₆ alkyl.

In embodiments, Y^{V2} is pyrazolyl optionally substituted with C₁-C₆ alkyl.

### Various Embodiments of Formula II, Formula IIa, and Formula IIb.

In one embodiment, n is 6 and each L forms the following LNK: (O)—(C₁-C₆ alkyl)-(fused bicyclic 4-10 membered heterocycloalkylene)-(C₁-C₆ alkyl)-(O)—(C₁-C₆ alkyl).

In one embodiment, n is 6 and each L forms the following LNK: (O)—(C₁-C₆ alkyl)-(fused bicyclic 8 membered heterocycloalkylene)-(C₁-C₆ alkyl)-(O)—(C₁-C₆ alkyl).

In one embodiment, n is 7 and each L forms the following LNK: (O)—(C₁-C₆ alkyl)-(fused bicyclic 4-10 membered heterocycloalkylene)-(C₁-C₆ alkyl)-(O)—C(O)-(monocyclic 4-membered heterocycloalkylene), wherein heterocycloalkylene is optionally substituted with one or two instances of C₁₋₆ alkyl.

In one embodiment, n is 7 and each L forms the following LNK: (O)—(C₁-C₆ alkyl)-(fused bicyclic 8 membered heterocycloalkylene)-(C₁-C₆ alkyl)-(O)—C(O)-(monocyclic 6 membered heterocycloalkylene), wherein heterocycloalkylene is optionally substituted with one or two instances of C₁₋₆ alkyl.

In one embodiment, n is 3 and each L forms the following LNK: (O)—(C₁-C₆ alkyl)-(spiro-fused 5-12 membered heterocycloalkylene).

In one embodiment, n is 3 and each L forms the following LNK: (O)—(C₁-C₆ alkyl)-(spiro-fused 9 membered heterocycloalkylene).

In one embodiment, n is 3 and each L forms the following LNK: (O)—(C₁-C₆ alkyl)-(fused bicyclic 4-10 membered heterocycloalkylene).

In one embodiment, n is 3 and each L forms the following LNK: (O)—(C₁-C₆ alkyl)-(fused bicyclic 8 membered heterocycloalkylene).

In one embodiment, n is 3 and each L forms the following LNK: (O)—(C₁-C₆ alkyl)-(fused bicyclic 9 membered heterocycloalkylene).

In one embodiment, n is 5 and each L forms the following LNK: (O)—(C₁-C₆ alkyl)-(fused bicyclic 4-10 membered heterocycloalkylene)-(C₁-C₆ alkyl)-(spiro-fused 5-12 membered heterocycloalkylene).

In one embodiment, n is 5 and each L forms the following LNK: (O)—(C₁-C₆ alkyl)-(fused bicyclic 8 membered heterocycloalkylene)-(C₁-C₆ alkyl)-(spiro-fused 9 membered heterocycloalkylene).

In one embodiment, n is 8 and each L forms the following LNK: (O)—(C₁-C₆ alkyl)-(fused bicyclic 4-10 membered heterocycloalkylene)-(C₁-C₆ alkyl)-(O)—C(O)—N(C₁-C₆ alkyl)-(C₁₋₆ alkyl).

In one embodiment, n is 8 and each L forms the following LNK: (O)—(C₁-C₆ alkyl)-(fused bicyclic 8 membered heterocycloalkylene)-(C₁-C₆ alkyl)-(O)—C(O)—N(C₁-C₆ alkyl)-(C₁-C₆ alkyl).

In one embodiment, n is 5 and each L forms the following LNK: (O)—(C₁-C₆ alkyl)-(fused bicyclic 4-10 membered heterocycloalkylene)-(C₁-C₆ alkyl)-(monocyclic 4-10 membered heterocycloalkylene).

In one embodiment, n is 5 and each L forms the following LNK: (O)—(C₁-C₆ alkyl)-(fused bicyclic 8 membered heterocycloalkylene)-(C₁-C₆ alkyl)-(monocyclic 6 membered heterocycloalkylene).

In one embodiment, n is 7 and each L forms the following LNK: (O)—(C₁-C₆ alkyl)-(fused bicyclic 4-10 membered heterocycloalkylene)-(C₁-C₆ alkyl)-(monocyclic 4-10 membered heterocycloalkylene)-(C₁-C₆ alkyl)-(O).

In one embodiment, n is 7 and each L forms the following LNK: (O)—(C₁-C₆ alkyl)-(fused bicyclic 8 membered heterocycloalkylene)-(C₁-C₆ alkyl)-(monocyclic 6 membered heterocycloalkylene)-(C₁-C₆ alkyl)-(O).

In one embodiment, n is 7 and each L forms the following LNK: (O)—(C₁-C₆ alkyl)-(fused bicyclic 4-10 membered heterocycloalkylene)-(C₁-C₆ alkyl)-(O)—C(O)-(spiro-fused 5-12 membered heterocycloalkylene).

In one embodiment, n is 7 and each L forms the following LNK: (O)—(C₁-C₆ alkyl)-(fused bicyclic 8 membered heterocycloalkylene)-(C₁-C₆ alkyl)-(O)—C(O)-(spiro-fused 7 membered heterocycloalkylene).

In one embodiment, n is 9 and each L forms the following LNK: (O)—(C₁-C₆ alkyl)-(fused bicyclic 4-10 membered heterocycloalkylene)-(C₁-C₆ alkyl)-(O)—C(O)-(monocyclic 4-10 membered heterocycloalkylene)-(C₁-C₆ alkyl)-(O).

In one embodiment, n is 9 and each L forms the following LNK: (O)—(C₁-C₆ alkyl)-(fused bicyclic 8 membered heterocycloalkylene)-(C₁-C₆ alkyl)-(O)—C(O)-(monocyclic 4 membered heterocycloalkylene)-(C₁-C₆ alkyl)-(O).

In one embodiment, n is 8 and each L forms the following LNK: (O)—(C₁-C₆ alkyl)-(fused bicyclic 4-10 membered heterocycloalkylene)-(C₁-C₆ alkyl)-(O)—C(O)-(monocyclic 4-10 membered heterocycloalkylene)-(O).

In one embodiment, n is 8 and each L forms the following LNK: (O)—(C₁-C₆ alkyl)-(fused bicyclic 8 membered heterocycloalkylene)-(C₁-C₆ alkyl)-(O)—C(O)-(monocyclic 6 membered heterocycloalkylene)-(O).

In one embodiment, n is 4 and each L forms the following LNK: (O)—(C₁-C₆ alkyl)-(monocyclic 4-10 membered heterocycloalkylene)-N(C₁-C₆ alkyl).

In one embodiment, n is 4 and each L forms the following LNK: (O)—(C₁-C₆ alkyl)-(monocyclic 6 membered heterocycloalkylene)-N(C₁-C₆ alkyl).

In one embodiment, n is 5 and each L forms the following LNK: (O)—(C₁-C₆ alkyl)-(monocyclic 4-10 membered heterocycloalkylene)-(C₁-C₆ alkyl)-(O), wherein heterocycloalkylene is optionally substituted with C₁-C₆ alkyl, O—(C₁-C₆ alkyl), and C₁-C₆ haloalkyl.

In one embodiment, n is 5 and each L forms the following LNK: (O)—(C₁-C₆ alkyl)-(monocyclic 6 membered heterocycloalkylene)-(C₁-C₆ alkyl)-(O), wherein heterocycloalkylene is optionally substituted with C₁-C₆ alkyl.

In one embodiment, n is 7 and each L forms the following LNK: (O)—(C₁-C₆ alkyl)-(monocyclic 4-10 membered heterocycloalkylene)-(C₁-C₆ alkyl)-(O)—C(O)-(monocyclic 4-10 membered heterocycloalkylene), wherein heterocycloalkylene is optionally substituted with C₁-C₆ alkyl, O—(C₁-C₆ alkyl), and C₁-C₆ haloalkyl.

In one embodiment, n is 7 and each L forms the following LNK: (O)—(C₁-C₆ alkyl)-(monocyclic 5 membered heterocycloalkylene)-(C₁₋₆ alkyl)-(O)—C(O)-(monocyclic 6 membered heterocycloalkylene), wherein heterocycloalkylene is optionally substituted with C₁-C₆ alkyl.

In one embodiment, n is 5 and each L forms the following LNK: (O)—(C₁-C₆ alkyl)-(monocyclic 4-10 membered heterocycloalkylene)-(C₁-C₆ alkyl)-(monocyclic 4-10 membered heterocycloalkylene), wherein heterocycloalkylene is optionally substituted with C₁-C₆ alkyl, O—(C₁-C₆ alkyl), and C₁-C₆ haloalkyl.

In one embodiment, n is 5 and each L forms the following LNK: (O)—(C₁-C₆ alkyl)-(monocyclic 6 membered heterocycloalkylene)-(C₁-C₆ alkyl)-(monocyclic 4 membered heterocycloalkylene).

In one embodiment, n is 4 and each L forms the following LNK: (O)—(C₁-C₆ alkyl)-(monocyclic 4-10 membered heterocycloalkylene)-(O).

In one embodiment, n is 4 and each L forms the following LNK: (O)—(C₁-C₆ alkyl)-(monocyclic 6 membered heterocycloalkylene)-(O).

In one embodiment, each L forms the following LNK:

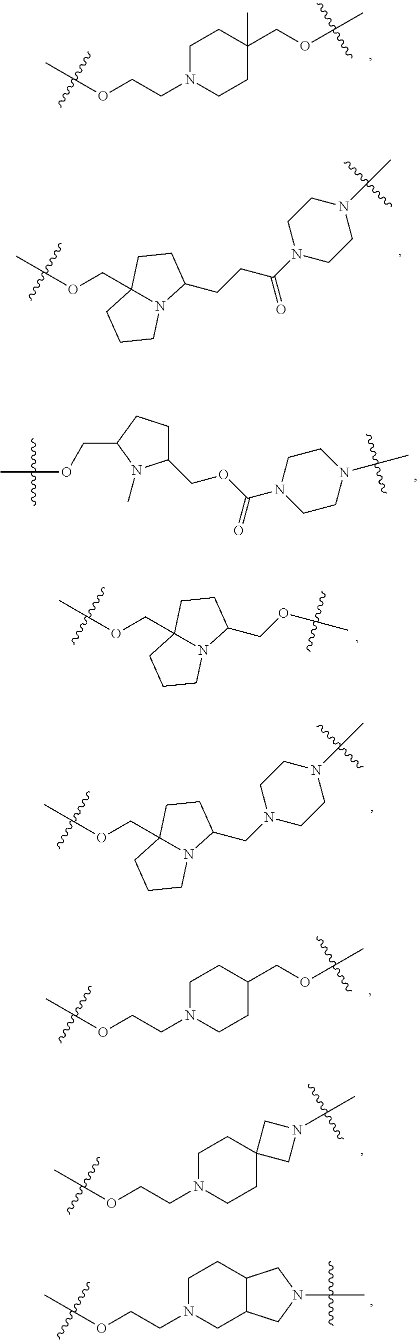

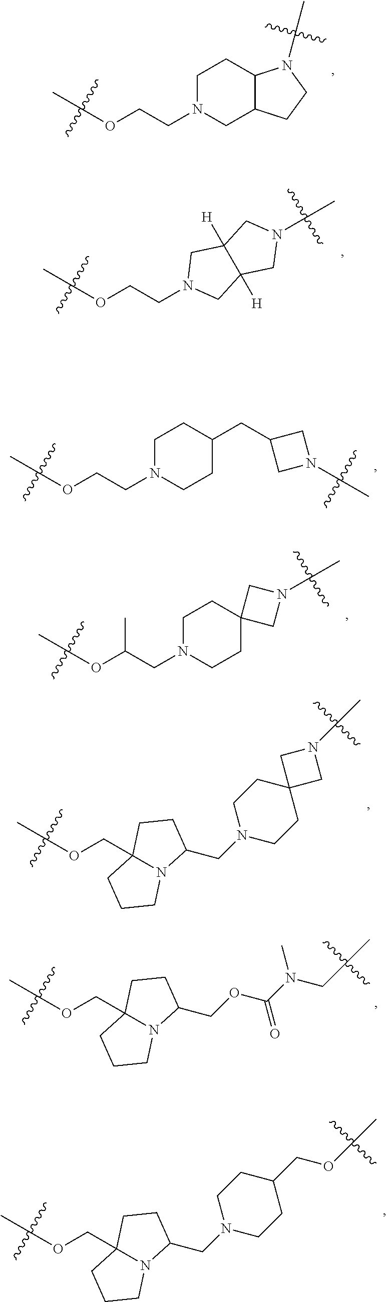

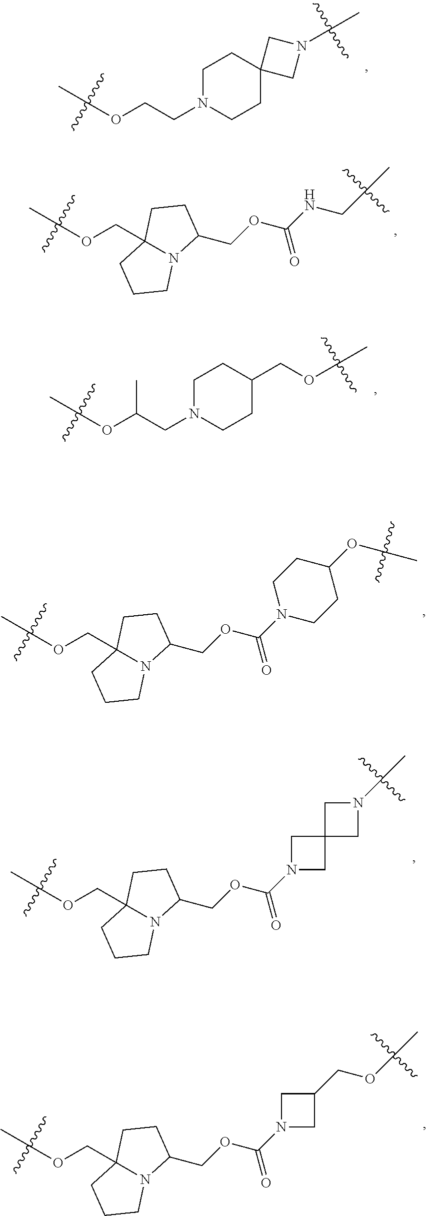 [SMILES: *OCCN1CCC2(CC1)CN(*)C2 ; *CNC(=O)OCC1CCC2(CO*)CCCN12 ; *OCC1CCN(CC(C)O*)CC1 ; *OCC12CCCN1C(COC(=O)N1CCC(O*)CC1)CC2 ; *OCC12CCCN1C(COC(=O)N1CC3(CN(*)C3)C1)CC2 ; *OCC1CN(C(=O)OCC2CCC3(CO*)CCCN23)C1]

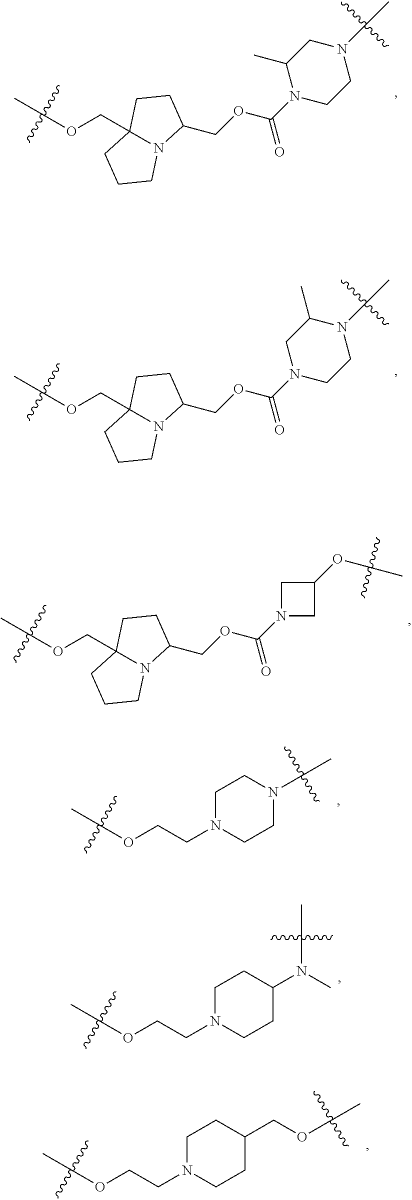 [SMILES: *OCC12CCCN1C(COC(=O)N1CCN(*)CC1C)CC2 ; *OCC12CCCN1C(COC(=O)N1CCN(*)C(C)C1)CC2 ; *OCC12CCCN1C(COC(=O)N1CC(O*)C1)CC2 ; *OCCN1CCN(*)CC1 ; *OCCN1CCC(N(*)C)CC1 ; *OCCN1CCC(CO*)CC1]

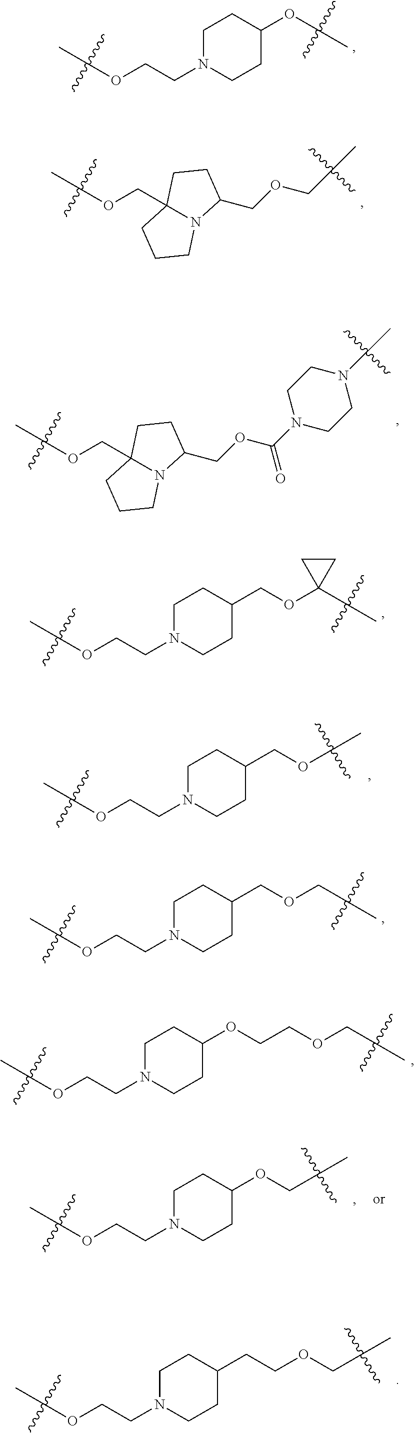

In another aspect, the application pertains to a compound, wherein the compound is:

| Cmpd. | | |
|---|---|---|
| No | Structure | Name |
| 1 | 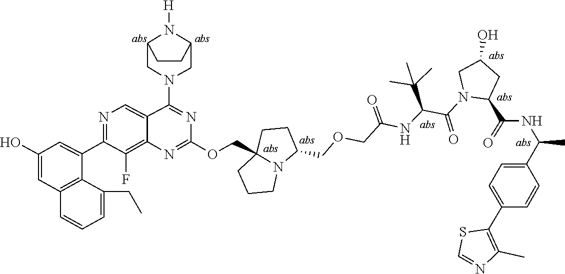 [SMILES: CCc1cccc2cc(O)cc(-c3ncc4c(N5CC6CCC(C5)N6)nc(OC[C@]56CCCN5[C@@H](COCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] | (2S,4R)-1-[(2S)-2-(2- {[(3R,7aR)-7a-{[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}acetamido)- 3,3-dimethylbutanoyl]-4- hydroxy-N-[(1S)-1-[4-(4- methyl-1,3-thiazol-5- yl)phenyl]ethyl] pyrrolidine- 2-carboxamide |
| 2 | 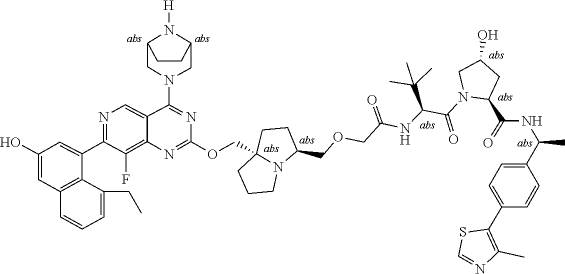 [SMILES: CCc1cccc2cc(O)cc(-c3ncc4c(N5CC6CCC(C5)N6)nc(OC[C@@]56CCCN5[C@H](COCC(=O)N[C@H](C(=O)N5C[C@H](O)C[C@H]5C(=O)N[C@@H](C)c5ccc(-c7scnc7C)cc5)C(C)(C)C)CC6)nc4c3F)c12] | (2S,4R)-1-[(2S)-2-(2- {[(3S,7aS)-7a-{[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}acetamido)- 3,3-dimethylbutanoyl]-4- hydroxy-N-[(1S)-1-[4-(4- methyl-1,3-thiazol-5- yl)phenyl]ethyl] pyrrolidine- 2-carboxamide |
| 3 | 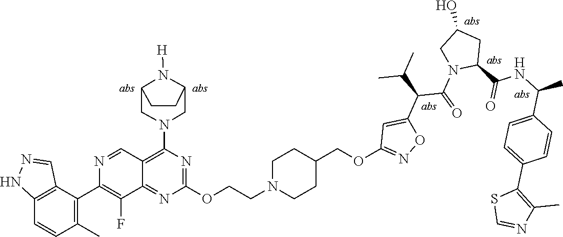 [SMILES: Cc1ccc2[nH]ncc2c1-c1ncc2c(N3CC4CCC(C3)N4)nc(OCCN3CCC(COc4cc([C@H](C(=O)N5C[C@H](O)C[C@H]5C(=O)N[C@@H](C)c5ccc(-c6scnc6C)cc5)C(C)C)on4)CC3)nc2c1F] | (2S,4R)-1-[(2R)-2-{3-[(1- {2-[(4-{3,8- diazabicyclo[3.2.1] octan-3-yl}-8- fluoro-7-(5-methyl- 1H-indazol-4- yl)pyrido[4,3-d]pyrimidin- 2-yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl] pyrrolidine- 2-carboxamide |
| 4 | 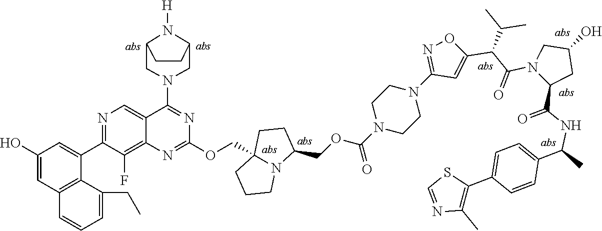 [SMILES: CCc1cccc2cc(O)cc(-c3ncc4c(N5CC6CCC(C5)N6)nc(OC[C@@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 5 | 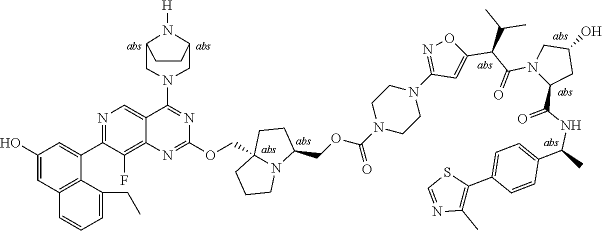 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 6 | 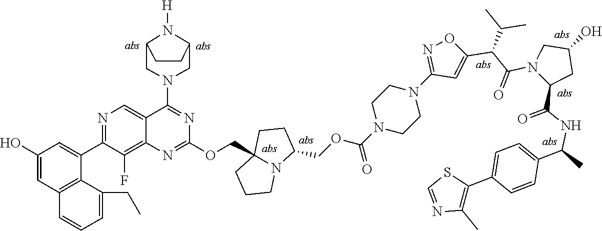 [SMILES: CCc1cccc2cc(O)cc(-c3ncc4c(N5CC6CCC(C5)N6)nc(OC[C@]56CCCN5[C@@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 7 | 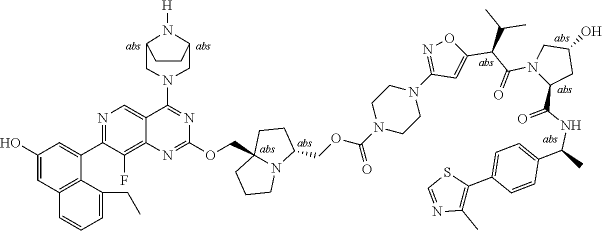 [SMILES: CCc1cccc2cc(O)cc(-c3ncc4c(N5CC6CCC(C5)N6)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 8 | 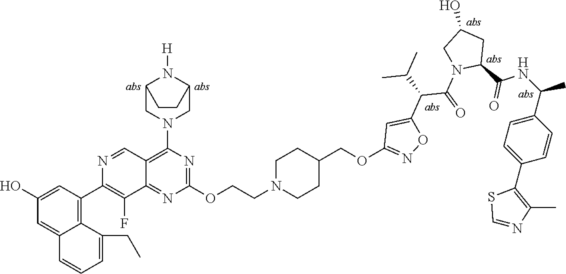 [SMILES: CCc1cccc2cc(O)cc(-c3ncc4c(N5CC6CCC(C5)N6)nc(OCCN5CCC(COc6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2S)-2-{3-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 9 | 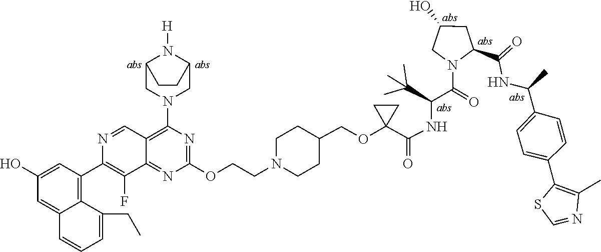 [SMILES: CCc1cccc2cc(O)cc(-c3ncc4c(N5CC6CCC(C5)N6)nc(OCCN5CCC(COC6(C(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12] | (2S,4R)-1-[(2S)-2-({1-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]cyclopropyl} formamido)-3,3- dimethylbutanoyl]-4- hydroxy-N-[(1S)-1-[4-(4- methyl-1,3-thiazol-5- yl)phenyl]ethyl]pyrrolidine- 2-carboxamide |
| 10 | 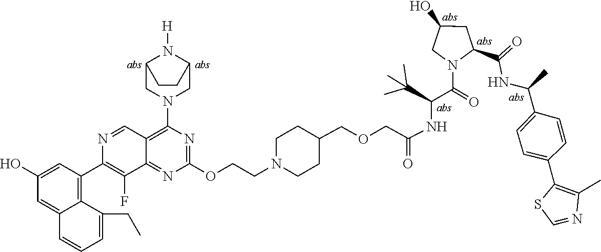 [SMILES: CCc1cccc2cc(O)cc(-c3ncc4c(N5CC6CCC(C5)N6)nc(OCCN5CCC(COCC(=O)N[C@H](C(=O)N6C[C@@H](O)C[C@H]6C(=O)N[C@@H](C)c6ccc(-c7scnc7C)cc6)C(C)(C)C)CC5)nc4c3F)c12] | (2S,4S)-1-[(2S)-2-{2-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]acetamido}- 3,3-dimethylbutanoyl]-4- hydroxy-N-[(1S)-1-[4-(4- methyl-1,3-thiazol-5- yl)phenyl]ethyl]pyrrolidine- 2-carboxamide |
| 11 | 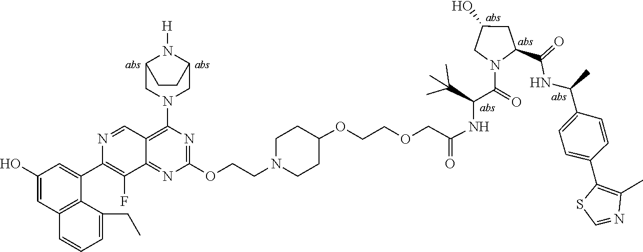 [SMILES: CCc1cccc2cc(O)cc(-c3ncc4c(N5CC6CCC(C5)N6)nc(OCCN5CCC(OCCOCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] | (2S,4R)-1-[(2S)-2-(2-{2- [(1-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)oxy]ethoxy}acetamido)- 3,3-dimethylbutanoyl]-4- hydroxy-N-[(1S)-1-[4-(4- methyl-1,3-thiazol-5- yl)phenyl]ethyl]pyrrolidine- 2-carboxamide |
| 12 | 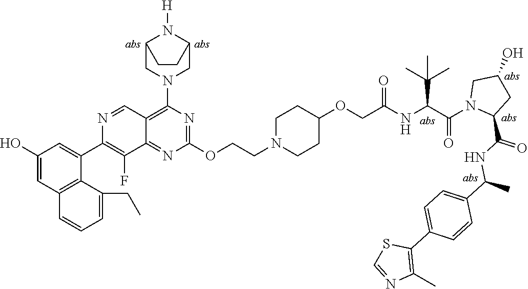 [SMILES: CCc1cccc2cc(O)cc(-c3ncc4c(N5CC6CCC(C5)N6)nc(OCCN5CCC(OCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] | (2S,4R)-1-[(2S)-2-{2-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)oxy]acetamido}-3,3- dimethylbutanoyl]-4- hydroxy-N-[(1S)-1-[4-(4- methyl-1,3-thiazol-5- yl)phenyl]ethyl]pyrrolidine- 2-carboxamide |
| 13 | 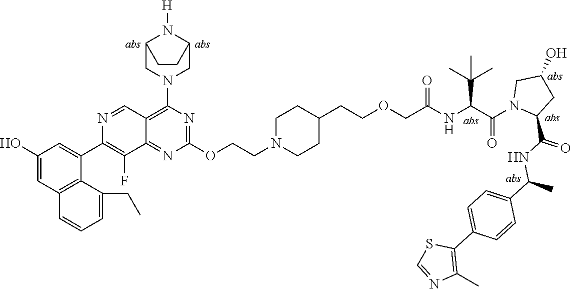 [SMILES: CCc1cccc2cc(O)cc(-c3ncc4c(N5CC6CCC(C5)N6)nc(OCCN5CCC(CCOCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] | (2S,4R)-1-[(2S)-2-{2-[2- (1-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)ethoxy]acetamido}-3,3- dimethylbutanoyl]-4- hydroxy-N-[(1S)-1-[4-(4- methyl-1,3-thiazol-5- yl)phenyl]ethyl]pyrrolidine- 2-carboxamide |
| 14 | 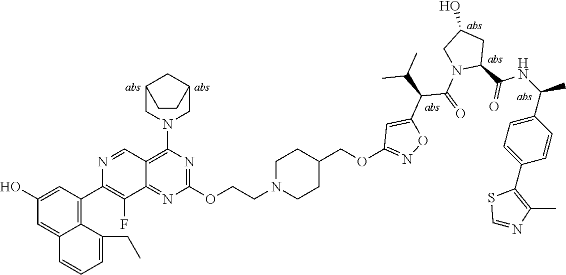 [SMILES: CCc1cccc2cc(O)cc(-c3ncc4c(N5CC6CCC(C6)C5)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3- azabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 15 | 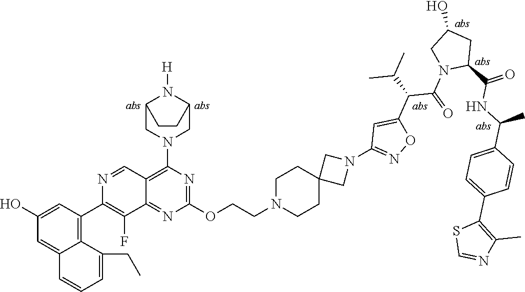 [SMILES: CCc1cccc2cc(O)cc(-c3ncc4c(N5CC6CCC(C5)N6)nc(OCCN5CCC6(CC5)CN(c5cc([C@@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2S)-2-[3-(7- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(4- methyl-1,3-thiazol-5- yl)phenyl]ethyl]pyrrolidine- 2-carboxamide |
| 16 | 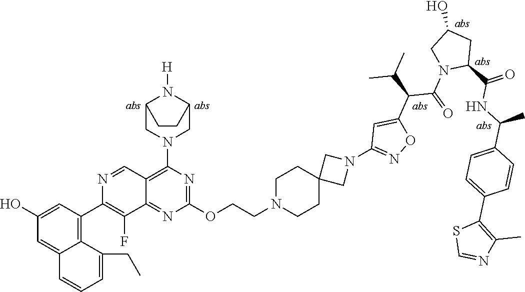 [SMILES: CCc1cccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2R)-2-[3-(7- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(4- methyl-1,3-thiazol-5- yl)phenyl]ethyl]pyrrolidine- 2-carboxamide |
| 17 | 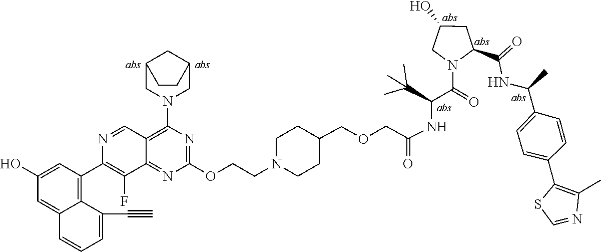 [SMILES: C#Cc1cccc2cc(O)cc(-c3ncc4c(N5CC6CCC(C6)C5)nc(OCCN5CCC(COCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] | (2S,4R)-1-[(2S)-2-{2-[(1- {2-[(4-{3- azabicyclo[3.2.1]octan-3- yl}-7-(8-ethynyl-3- hydroxynaphthalen-1-yl)- 8-fluoropyrido[4,3- d]pyrimidin-2- yl)oxy]ethyl}piperidin-4- yl)methoxy]acetamido}- 3,3-dimethylbutanoyl]-4- hydroxy-N-[(1S)-1-[4-(4- methyl-1,3-thiazol-5- yl)phenyl]ethyl]pyrrolidine- 2-carboxamide |
| 18 | 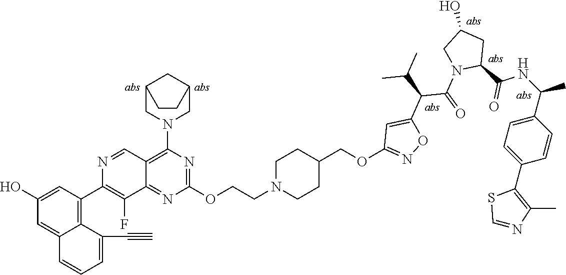 [SMILES: C#Cc1cccc2cc(O)cc(-c3ncc4c(N5CC6CCC(C6)C5)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3- azabicyclo[3.2.1]octan-3- yl}-7-(8-ethynyl-3- hydroxynaphthalen-1-yl)- 8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 19 | 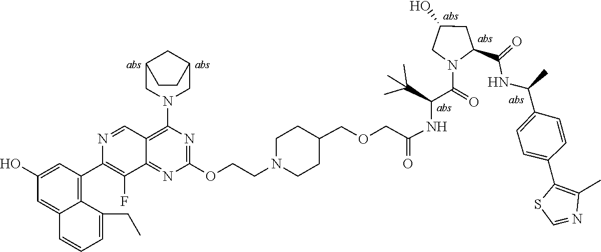 [SMILES: CCc1cccc2cc(O)cc(-c3ncc4c(N5CC6CCC(C6)C5)nc(OCCN5CCC(COCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] | (2S,4R)-1-[(2S)-2-{2-[(1- {2-[(4-{3- azabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]acetamido}- 3,3-dimethylbutanoyl]-4- hydroxy-N-[(1S)-1-[4-(4- methyl-1,3-thiazol-5- yl)phenyl]ethyl]pyrrolidine- 2-carboxamide |
| 20 | 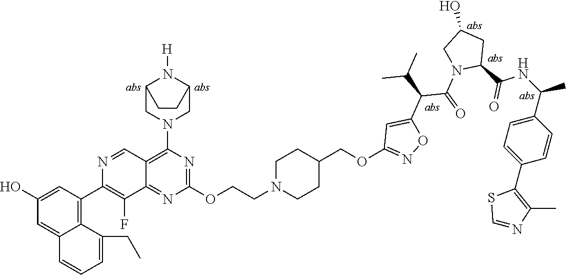 [SMILES: CCc1cccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 21 | 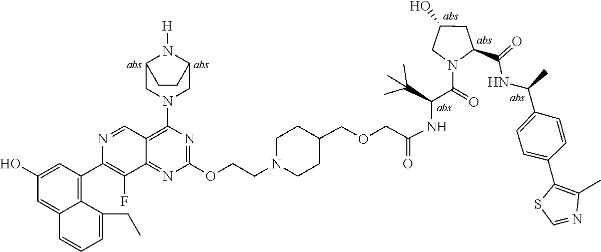 [SMILES: CCc1cccc2cc(O)cc(-c3ncc4c(N5CC6CCC(C5)N6)nc(OCCN5CCC(COCC(=O)N[C@H](C(=O)N6C[C@H](O)C[C@H]6C(=O)N[C@@H](C)c6ccc(-c7scnc7C)cc6)C(C)(C)C)CC5)nc4c3F)c12] | (2S,4R)-1-[(2S)-2-{2-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]acetamido}- 3,3-dimethylbutanoyl]-4- hydroxy-N-[(1S)-1-[4-(4- methyl-1,3-thiazol-5- yl)phenyl]ethyl]pyrrolidine- 2-carboxamide |
| 22 | 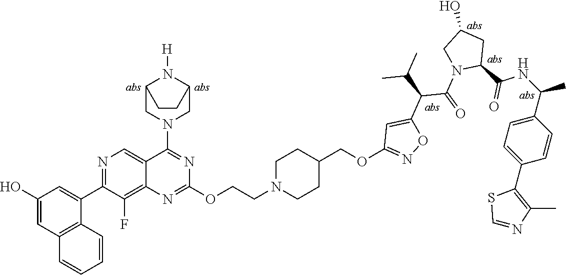 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc7ccccc67)c(F)c5n4)CC3)no2)C(C)C)cc1] | (2S,4R)-1-[(2R)-2-{3-[(1- {2-[(4-{3,8- diazabicyclo[3.2.1]octan-3- yl}-8-fluoro-7-(3- hydroxynaphthalen-1- yl)pyrido[4,3-d]pyrimidin- 2-yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 23 | 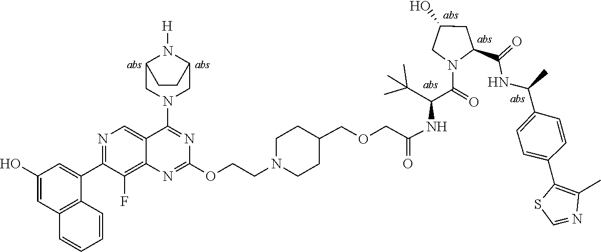 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CC5CCC(C4)N5)c4cnc(-c5cc(O)cc6ccccc56)c(F)c4n3)CC2)C(C)(C)C)cc1] | (2S,4R)-1-[(2S)-2-{2-[(1- {2-[(4-{3,8- diazabicyclo[3.2.1]octan-3- yl}-8-fluoro-7-(3- hydroxynaphthalen-1- yl)pyrido[4,3-d]pyrimidin- 2-yl)oxy]ethyl}piperidin-4- yl)methoxy]acetamido}- 3,3-dimethylbutanoyl]-4- hydroxy-N-[(1S)-1-[4-(4- methyl-1,3-thiazol-5- yl)phenyl]ethyl]pyrrolidine- 2-carboxamide |
| 24 | 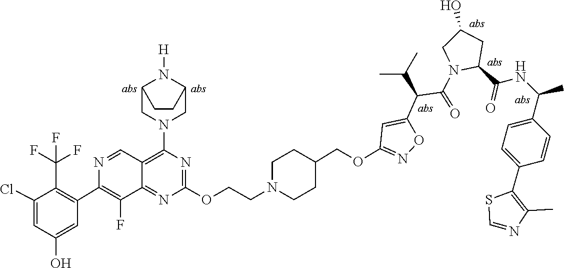 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc(Cl)c6C(F)(F)F)c(F)c5n4)CC3)no2)C(C)C)cc1] | (2S,4R)-1-[(2R)-2-[3-({1- [2-({7-[3-chloro-5- hydroxy-2- (trifluoromethyl)phenyl]-4- {3,8- diazabicyclo[3.2.1]octan-3- yl}-8-fluoropyrido[4,3- d]pyrimidin-2- yl}oxy)ethyl]piperidin-4- yl}methoxy)-1,2-oxazol-5- yl]-3-methylbutanoyl]-4- hydroxy-N-[(1S)-1-[4-(4- methyl-1,3-thiazol-5- yl)phenyl]ethyl]pyrrolidine- 2-carboxamide |
| 25 | 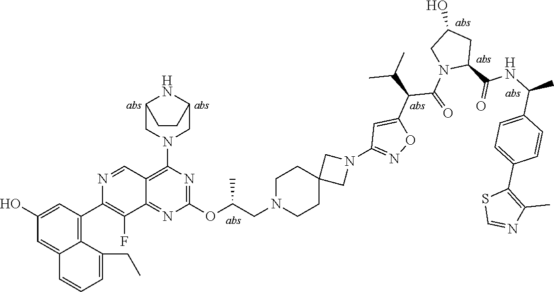 [SMILES: CCc1cccc2cc(O)cc(-c3ncc4c(N5CC6CCC(C5)N6)nc(O[C@H](C)CN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2R)-2-(3-{7- [(2R)-2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]propyl]-2,7- diazaspiro[3.5]nonan-2- yl}-1,2-oxazol-5-yl)-3- methylbutanoyl]-4- hydroxy-N-[(1S)-1-[4-(4- methyl-1,3-thiazol-5- yl)phenyl]ethyl]pyrrolidine- 2-carboxamide |
| 26 | 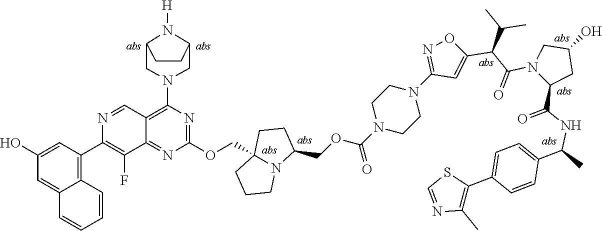 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@]5(COc6nc(N7CC8CCC(C7)N8)c7cnc(-c8cc(O)cc9ccccc89)c(F)c7n6)CCCN45)CC3)no2)C(C)C)cc1] | (2S,4R)-1-[(2R)-2-[3-(7- {2-[(4-{3,8- diazabicyclo[3.2.1]octan-3- yl}-7-(8-ethynyl-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(4- methyl-1,3-thiazol-5- yl)phenyl]ethyl] |
| | | pyrrolidine- |
| | | 2-carboxamide |
| 27 | 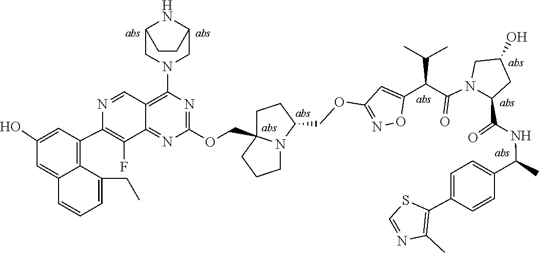 [SMILES: CCc1cccc2cc(O)cc(-c3ncc4c(N5CC6CCC(C5)N6)nc(OC[C@]56CCCN5[C@@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl] pyrrolidine- 2-carboxamide |
| 28 | 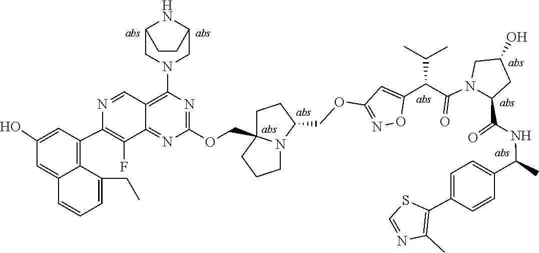 [SMILES: CCc1cccc2cc(O)cc(-c3ncc4c(N5CC6CCC(C5)N6)nc(OC[C@]56CCCN5[C@@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 29 | 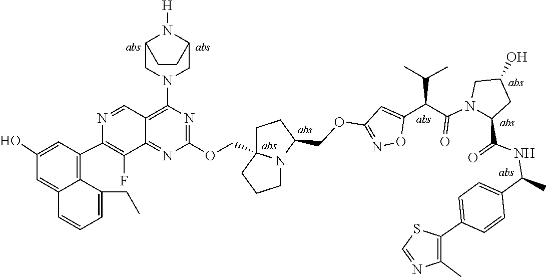 [SMILES: CCc1cccc2cc(O)cc(-c3ncc4c(N5CC6CCC(C5)N6)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 30 | 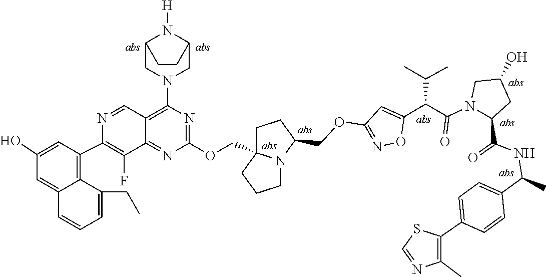 [SMILES: CCc1cccc2cc(O)cc(-c3ncc4c(N5CC6CCC(C5)N6)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 31 | 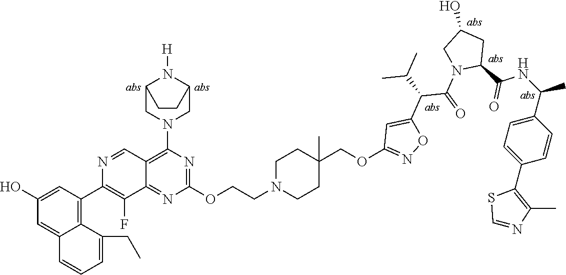 [SMILES: CCc1cccc2cc(O)cc(-c3ncc4c(N5CC6CCC(C5)N6)nc(OCCN5CCC(C)(COc6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2S)-2-{3-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-4- methylpiperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 32 | 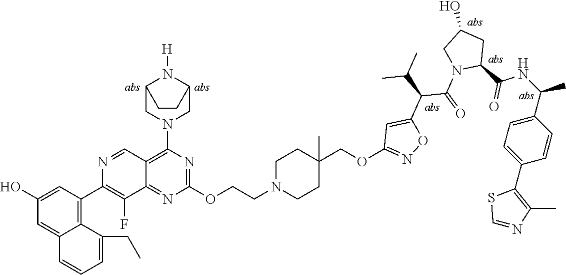 [SMILES: CCc1cccc2cc(O)cc(-c3ncc4c(N5CC6CCC(C5)N6)nc(OCCN5CCC(C)(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-4- methylpiperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 33 | 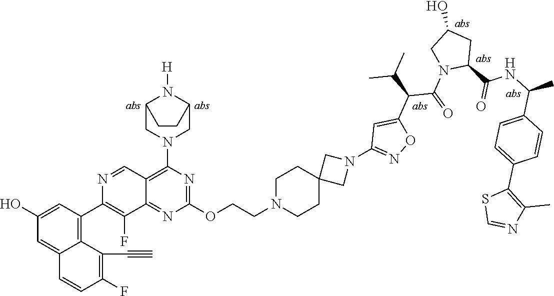 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2R)-2-[3-(7- {2-[(4-{3,8- diazabicyclo[3.2.1]octan-3- yl}-7-(8-ethynyl-7-fluoro- 3-hydroxynaphthalen-1- yl)-8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(4- methyl-1,3-thiazol-5- yl)phenyl]ethyl]pyrrolidine- 2-carboxamide |
| 34 | 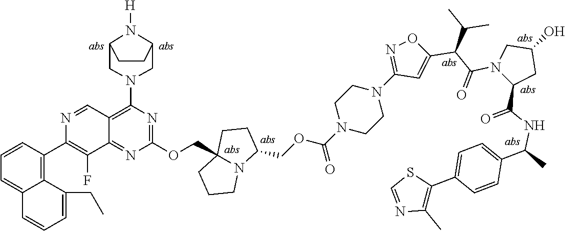 [SMILES: CCc1cccc2cccc(-c3ncc4c(N5CC6CCC(C5)N6)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aR)-7a-{[(4-{3,8- diazabicyclo[3.2.1]octan-3- yl}-7-(8-ethylnaphthalen- 1-yl)-8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}piperazine-1- |
| | | carboxylate |
| 35 | 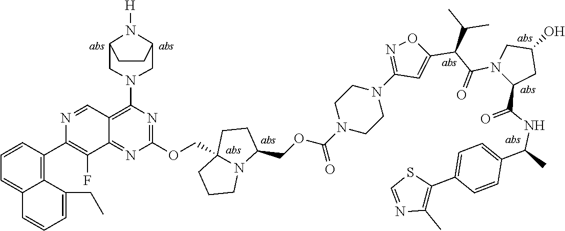 [SMILES: CCc1cccc2cccc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aS)-7a-{[(4-{3,8- diazabicyclo[3.2.1]octan-3- yl}-7-(8-ethylnaphthalen- 1-yl)-8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}piperazine-1- |
| | | carboxylate |
| 36 | 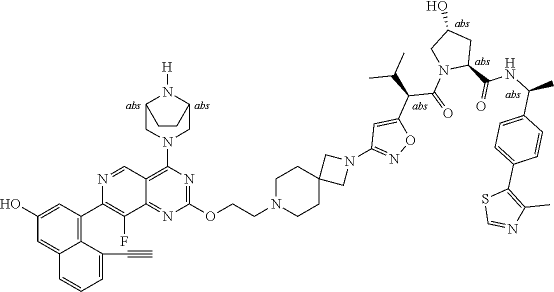 [SMILES: C#Cc1cccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2R)-2-[3-(7- {2-[(4-{3,8- diazabicyclo[3.2.1]octan-3- yl}-7-(8-ethynyl-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(4- methyl-1,3-thiazol-5- yl)phenyl]ethyl]pyrrolidine- 2-carboxamide |
| 37 | 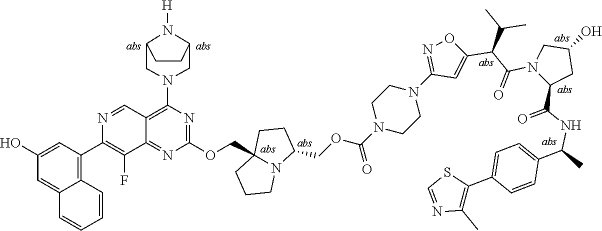 [SMILES: Cc1ncsc1-c1ccc([C@H](C)NC(=O)[C@@H]2C[C@@H](O)CN2C(=O)[C@@H](c2cc(N3CCN(C(=O)OC[C@H]4CC[C@@]5(COc6nc(N7CC8CCC(C7)N8)c7cnc(-c8cc(O)cc9ccccc89)c(F)c7n6)CCCN45)CC3)no2)C(C)C)cc1] | [(3R,7aR)-7a-{[(4-{3,8- diazabicyclo[3.2.1]octan-3- yl}-8-fluoro-7-(3- hydroxynaphthalen-1- yl)pyrido[4,3-d]pyrimidin- 2-yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}piperazine-1- |
| | | carboxylate |
| 38 | 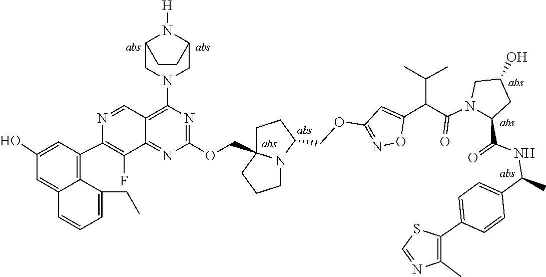 [SMILES: CCc1cccc2cc(O)cc(-c3ncc4c(N5CC6CCC(C5)N6)nc(OC[C@]56CCCN5[C@@H](COc5cc(C(C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[2-(3- {[(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 39 | 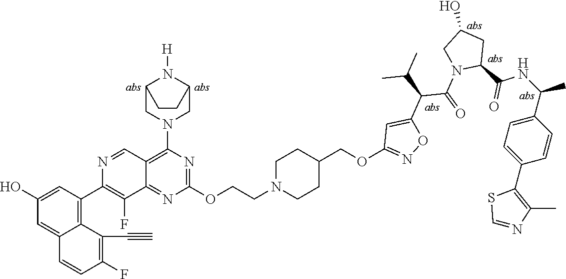 [SMILES: C#Cc1c(F)ccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3,8- diazabicyclo[3.2.1]octan-3- yl}-7-(8-ethynyl-7-fluoro- 3-hydroxynaphthalen-1- yl)-8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 40 | 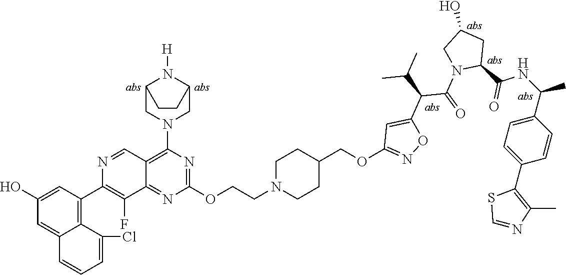 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc7cccc(Cl)c67)c(F)c5n4)CC3)no2)C(C)C)cc1] | (2S,4R)-1-[(2R)-2-(3-{[1- (2-{[7-(8-chloro-3- hydroxynaphthalen-1-yl)- 4-{3,8- diazabicyclo[3.2.1]octan-3- yl}-8-fluoropyrido[4,3- d]pyrimidin-2- yl]oxy}ethyl)piperidin-4- yl]methoxy}-1,2-oxazol-5- yl)-3-methylbutanoyl]-4- hydroxy-N-[(1S)-1-[4-(4- methyl-1,3-thiazol-5- yl)phenyl]ethyl]pyrrolidine- 2-carboxamide |
| 41 | 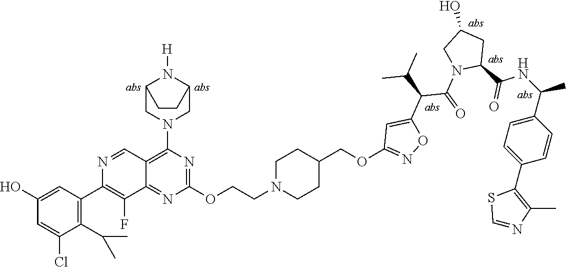 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc(Cl)c6C(C)C)c(F)c5n4)CC3)no2)C(C)C)cc1] | (2S,4R)-1-[(2R)-2-[3-({1- [2-({7-[3-chloro-5- hydroxy-2-(propan-2- yl)phenyl]-4-{3,8- diazabicyclo[3.2.1]octan-3- yl}-8-fluoropyrido[4,3- d]pyrimidin-2- yl}oxy)ethyl]piperidin-4- yl}methoxy)-1,2-oxazol-5- yl]-3-methylbutanoyl]-4- hydroxy-N-[(1S)-1-[4-(4- methyl-1,3-thiazol-5- yl)phenyl]ethyl]pyrrolidine- 2-carboxamide |
| 42 | 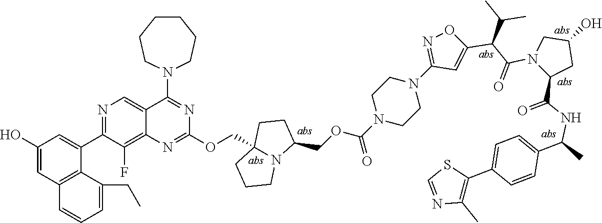 [SMILES: CCc1cccc2cc(O)cc(-c3ncc4c(N5CCCCCC5)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aS)-7a-({[4-(azepan- 1-yl)-7-(8-ethyl-3- hydroxynaphthalen-1-yl)- 8-fluoropyrido[4,3- d]pyrimidin-2- yl]oxy}methyl)- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 43 | 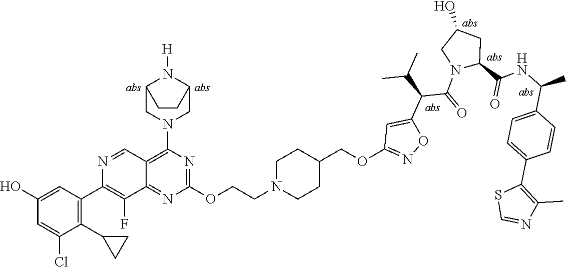 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc(Cl)c6C6CC6)c(F)c5n4)CC3)no2)C(C)C)cc1] | (2S,4R)-1-[(2R)-2-(3-{[1- (2-{[7-(3-chloro-2- cyclopropyl-5- hydroxyphenyl)-4-{3,8- diazabicyclo[3.2.1]octan-3- yl}-8-fluoropyrido[4,3- d]pyrimidin-2- yl]oxy}ethyl)piperidin-4- yl]methoxy}-1,2-oxazol-5- yl)-3-methylbutanoyl]-4- hydroxy-N-[(1S)-1-[4-(4- methyl-1,3-thiazol-5- yl)phenyl]ethyl]pyrrolidine- 2-carboxamide |
| 44 | 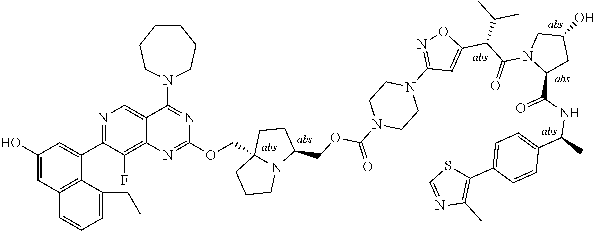 [SMILES: CCc1cccc2cc(O)cc(-c3ncc4c(N5CCCCCC5)nc(OC[C@@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aS)-7a-({[4-(azepan- 1-yl)-7-(8-ethyl-3- hydroxynaphthalen-1-yl)- 8-fluoropyrido[4,3- d]pyrimidin-2- yl]oxy}methyl)- hexahydro-1H-pyrrolizin- 3-yl]methyl 4-{5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}piperazine-1- |
| | | carboxylate |
| 45 | 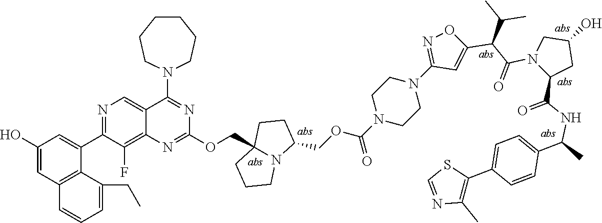 [SMILES: CCc1cccc2cc(O)cc(-c3ncc4c(N5CCCCCC5)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aR)-7a-({[4-(azepan- 1-yl)-7-(8-ethyl-3- hydroxynaphthalen-1-yl)- 8-fluoropyrido[4,3- d]pyrimidin-2- yl]oxy}methyl)- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 46 | 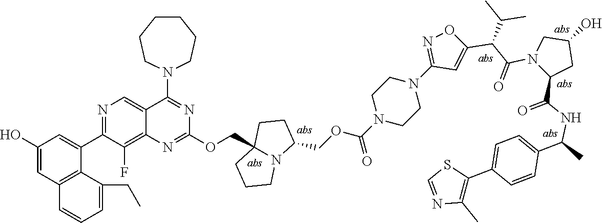 [SMILES: CCc1cccc2cc(O)cc(-c3ncc4c(N5CCCCCC5)nc(OC[C@]56CCCN5[C@@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aR)-7a-({[4-(azepan- 1-yl)-7-(8-ethyl-3- hydroxynaphthalen-1-yl)- 8-fluoropyrido[4,3- d]pyrimidin-2- yl]oxy}methyl)- hexahydro-1H-pyrrolizin- 3-yl]methyl 4-{5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}piperazine-1- |
| | | carboxylate |
| 47 | 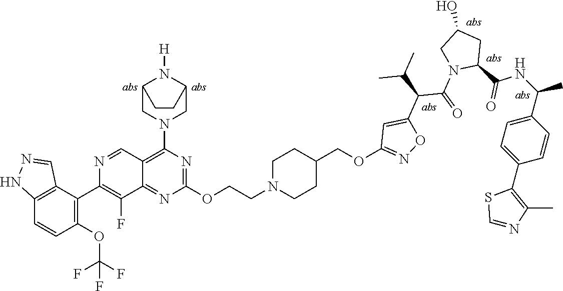 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6c(OC(F)(F)F)ccc7[nH]ncc67)c(F)c5n4)CC3)no2)C(C)C)cc1] | (2S,4R)-1-[(2R)-2-{3-[(1- {2-[(4-{3,8- diazabicyclo[3.2.1]octan-3- yl}-8-fluoro-7-[5- (trifluoromethoxy)-1H- indazol-4-yl]pyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 48 | 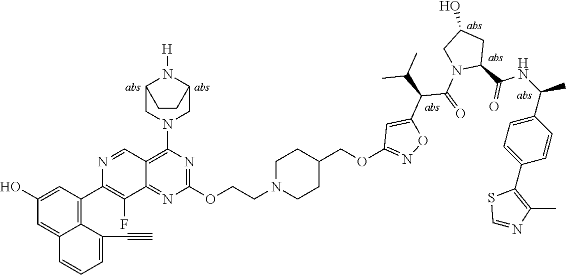 [SMILES: C#Cc1cccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3,8- diazabicyclo[3.2.1]octan-3- yl}-7-(8-ethynyl-3- hydroxynaphthalen-1-yl)- 8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 49 | 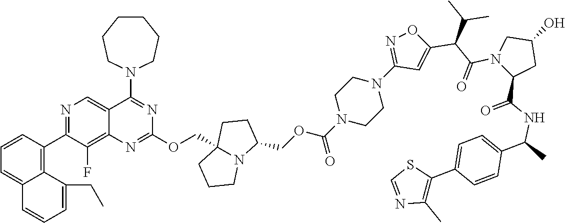 [SMILES: CCc1cccc2cccc(-c3ncc4c(N5CCCCCC5)nc(OC[C@@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aR)-7a-{[4-(azepan- 1-yl)-7-(8-ethylnaphthalen- 1-yl)-8-fluoropyrido[4,3- d]pyrimidin-2-yl]oxy}- hexahydro-1H-pyrrolizin- 3-yl]methyl 4-{5-[(2R*)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}piperazine-1- carboxylate |
| 50 | 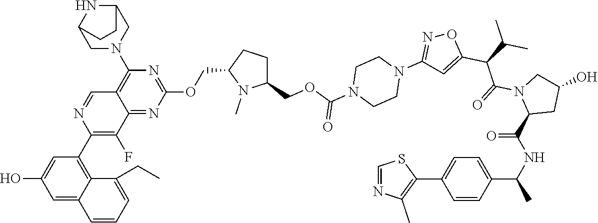 [SMILES: CCc1cccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12] | [(2S,5S)-5-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- |
| | | 1-oxobutan-2-yl]-1,2- |
| | | oxazol-3-yl}piperazine-1- |
| | | carboxylate |
| 51 | 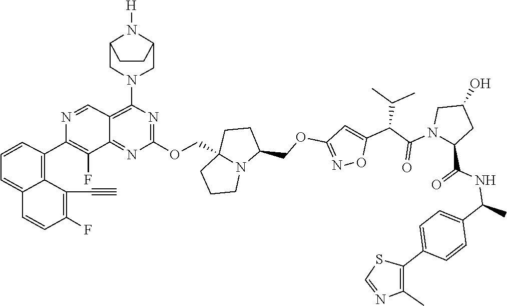 [SMILES: C#Cc1c(F)ccc2cccc(-c3ncc4c(N5CC6CCC(C5)N6)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aS)-7a-{[(4-{3,8- diazabicyclo[3.2.1]octan-3- yl}-7-(8-ethynyl-7- fluoronaphthalen-1-yl)-8- fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 52 | 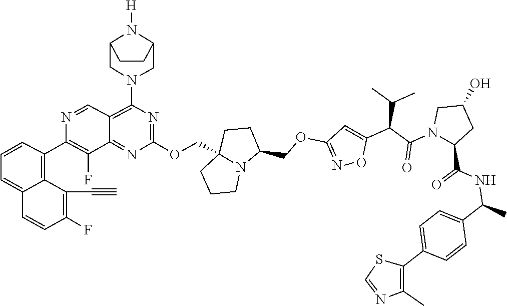 [SMILES: C#Cc1c(F)ccc2cccc(-c3ncc4c(N5CC6CCC(C5)N6)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aS)-7a-{[(4-{3,8- diazabicyclo[3.2.1]octan-3- yl}-7-(8-ethynyl-7- fluoronaphthalen-1-yl)-8- fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 53 | 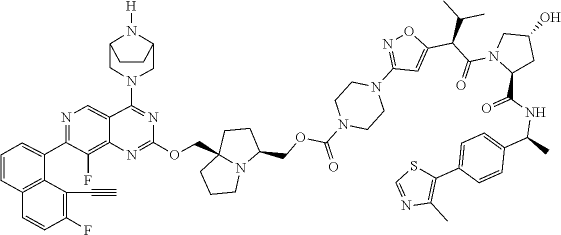 [SMILES: C#Cc1c(F)ccc2cccc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-{[(4-{3,8- diazabicyclo[3.2.1]octan-3- yl}-7-(8-ethynyl-7- fluoronaphthalen-1-yl)-8- fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 54 | 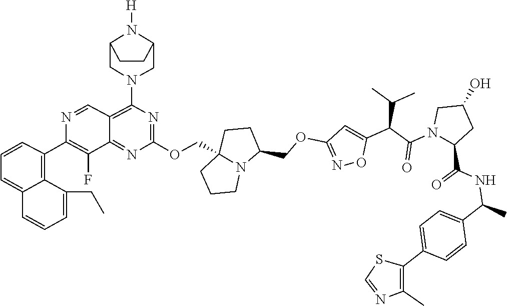 [SMILES: CCc1cccc2cccc(-c3ncc4c(N5CC6CCC(C5)N6)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aS)-7a-{[(4-{3,8- diazabicyclo[3.2.1]octan-3- yl}-7-(8-ethylnaphthalen- 1-yl)-8-fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 55 | 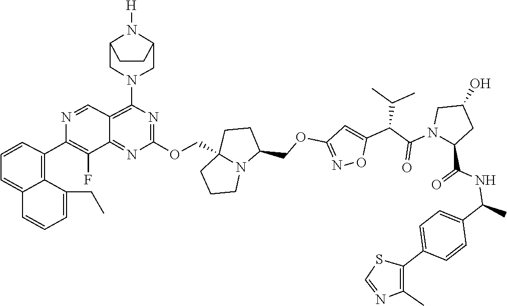 [SMILES: CCc1cccc2cccc(-c3ncc4c(N5CC6CCC(C5)N6)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aS)-7a-{[(4-{3,8- diazabicyclo[3.2.1]octan-3- yl}-7-(8-ethylnaphthalen- 1-yl)-8-fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 56 | 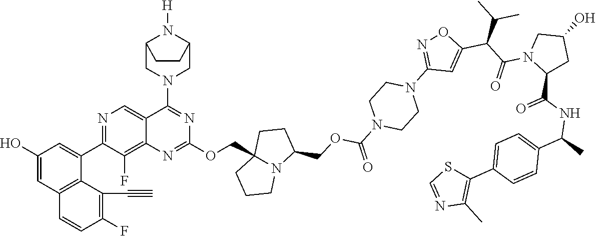 [SMILES: C#Cc1c(F)ccc2cc(O)cc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-{[(4-{3,8- diazabicyclo[3.2.1]octan-3- yl}-7-(8-ethynyl-7-fluoro- 3-hydroxynaphthalen-1- yl)-8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 57 | 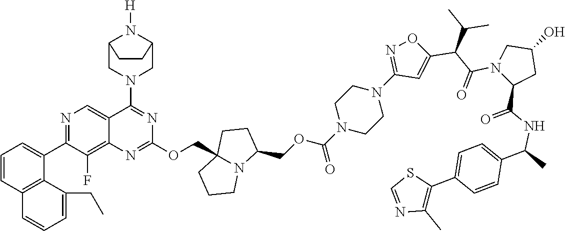 [SMILES: CCc1cccc2cccc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-{[(4-{3,8- diazabicyclo[3.2.1]octan-3- yl}-7-(8-ethylnaphthalen- 1-yl)-8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}piperazine-1- |
| | | carboxylate |
| 58 | 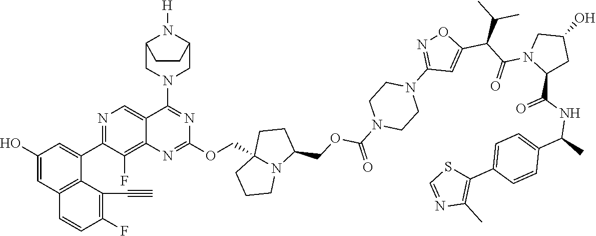 [SMILES: C#Cc1c(F)ccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aS)-7a-{[(4-{3,8- diazabicyclo[3.2.1]octan-3- yl}-7-(8-ethynyl-7-fluoro- 3-hydroxynaphthalen-1- yl)-8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 59 | 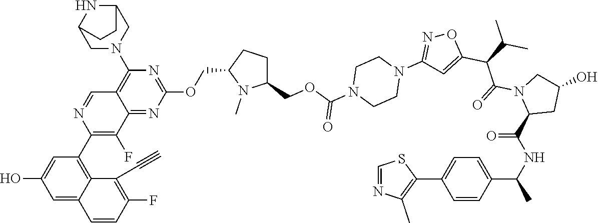 [SMILES: C#Cc1c(F)ccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12] | [(2S,5S)-5-{[(4-{3,8- diazabicyclo[3.2.1]octan-3- yl}-7-(8-ethynyl-7-fluoro- 3-hydroxynaphthalen-1- yl)-8-fluoropyrido[4,3- d]pyrimidin-2- yl)oxy]methyl}-1- methylpyrrolidin-2- yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- |
| | | 1-oxobutan-2-yl]-1,2- |
| | | oxazol-3-yl}piperazine-1- |
| | | carboxylate |
| 60 | 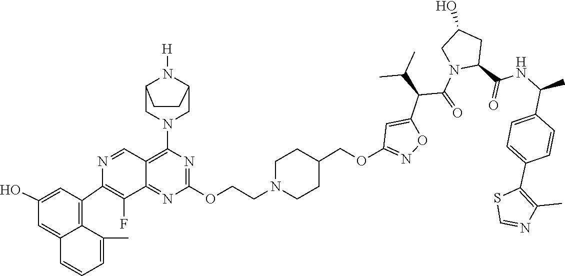 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc7cccc(C)c67)c(F)c5n4)CC3)no2)C(C)C)cc1] | (2S,4R)-1-[(2R)-2-{3-[(1- {2-[(4-{3,8- diazabicyclo[3.2.1]octan-3- yl}-8-fluoro-7-(3-hydroxy- 8-methylnaphthalen-1- yl)pyrido[4,3-d]pyrimidin- 2-yl)oxy]ethyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- 2-carboxamide |
| 61 | 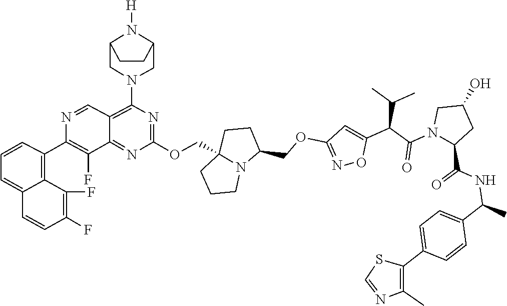 [SMILES: Cc1ncsc1-c1ccc([C@H](C)NC(=O)[C@@H]2C[C@@H](O)CN2C(=O)[C@@H](c2cc(OC[C@@H]3CC[C@]4(COc5nc(N6CC7CCC(C6)N7)c6cnc(-c7cccc8ccc(F)c(F)c78)c(F)c6n5)CCCN34)no2)C(C)C)cc1] | (2S,4R)-1-[(2R)-2-(3- {[(3S,7aS)-7a-{[(4-{3,8- diazabicyclo[3.2.1]octan-3- yl}-7-(7,8- difluoronaphthalen-1-yl)-8- fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 62 | 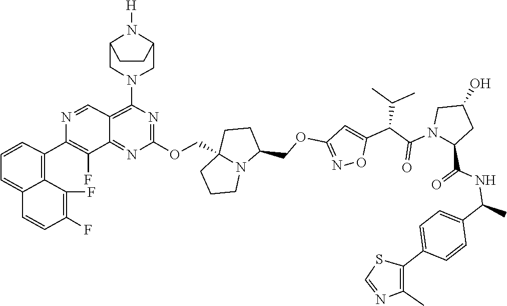 [SMILES: Cc1ncsc1-c1ccc([C@H](C)NC(=O)[C@@H]2C[C@@H](O)CN2C(=O)[C@H](c2cc(OC[C@@H]3CC[C@]4(COc5nc(N6CC7CCC(C6)N7)c6cnc(-c7cccc8ccc(F)c(F)c78)c(F)c6n5)CCCN34)no2)C(C)C)cc1] | (2S,4R)-1-[(2S)-2-(3- {[(3S,7aS)-7a-{[(4-{3,8- diazabicyclo[3.2.1]octan-3- yl}-7-(7,8- difluoronaphthalen-1-yl)-8- fluoropyrido[4,3- d]pyrimidin-2- yl)oxy]methyl}- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 63 | 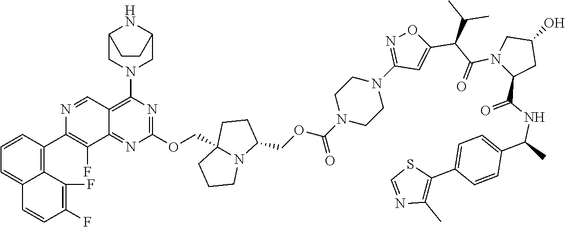 [SMILES: Cc1ncsc1-c1ccc([C@H](C)NC(=O)[C@@H]2C[C@@H](O)CN2C(=O)[C@@H](c2cc(N3CCN(C(=O)OC[C@H]4CC[C@]5(COc6nc(N7CC8CCC(C7)N8)c7cnc(-c8cccc9ccc(F)c(F)c89)c(F)c7n6)CCCN45)CC3)no2)C(C)C)cc1] | [(3R,7aS)-7a-{[(4-{3,8- diazabicyclo[3.2.1]octan-3- yl}-7-(7,8- difluoronaphthalen-1-yl)-8- fluoropyrido[4,3- d]pyrimidin-2- yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 64 | 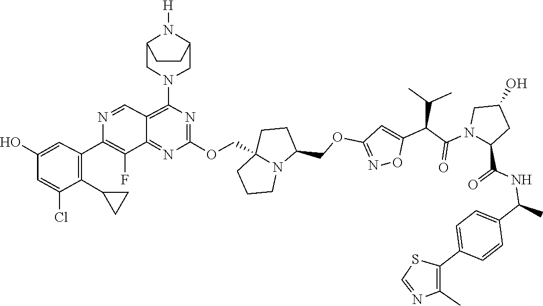 [SMILES: Cc1ncsc1-c1ccc([C@H](C)NC(=O)[C@@H]2C[C@@H](O)CN2C(=O)[C@@H](c2cc(OC[C@@H]3CC[C@]4(COc5nc(N6CC7CCC(C6)N7)c6cnc(-c7cc(O)cc(Cl)c7C7CC7)c(F)c6n5)CCCN34)no2)C(C)C)cc1] | (2S,4R)-1-[(2R)-2-(3- {[(3S,7aS)-7a-({[7-(3- chloro-2-cyclopropyl-5- hydroxyphenyl)-4-{3,8- diazabicyclo[3.2.1]octan-3- yl}-8-fluoropyrido[4,3- d]pyrimidin-2- yl]oxy}methyl)- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 65 | 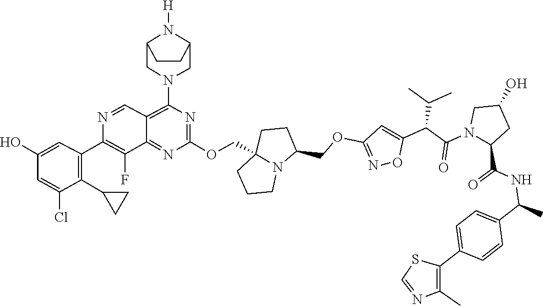 [SMILES: Cc1ncsc1-c1ccc([C@H](C)NC(=O)[C@@H]2C[C@@H](O)CN2C(=O)[C@H](c2cc(OC[C@@H]3CC[C@]4(COc5nc(N6CC7CCC(C6)N7)c6cnc(-c7cc(O)cc(Cl)c7C7CC7)c(F)c6n5)CCCN34)no2)C(C)C)cc1] | (2S,4R)-1-[(2S)-2-(3- {[(3S,7aS)-7a-({[7-(3- chloro-2-cyclopropyl-5- hydroxyphenyl)-4-{3,8- diazabicyclo[3.2.1]octan-3- yl}-8-fluoropyrido[4,3- d]pyrimidin-2- yl]oxy}methyl)- hexahydro-1H-pyrrolizin- 3-yl]methoxy}-1,2-oxazol- 5-yl)-3-methylbutanoyl]-4- hydroxy-N-[(1S)-1-[4-(4- methyl-1,3-thiazol-5- yl)phenyl]ethyl]pyrrolidine- 2-carboxamide |
| 66 | 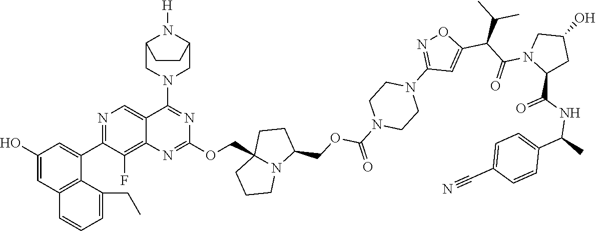 [SMILES: CCc1cccc2cc(O)cc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(C#N)cc8)C(C)C)on7)CC5)CC6)nc4c3F)c12] | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R)-1- [(2S,4R)-2-{[(1S)-1-(4- cyanophenyl)ethyl] carbamoyl}- 4-hydroxypyrrolidin- 1-yl]-3-methyl-1- oxobutan-2-yl]-1,2-oxazol- |
| | | 3-yl}piperazine-1- |
| | | carboxylate |
| 67 | 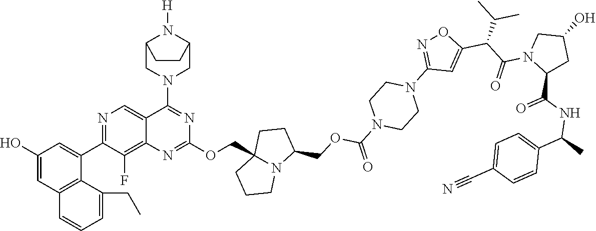 [SMILES: CCc1cccc2cc(O)cc(-c3ncc4c(N5CC6CCC(C5)N6)nc(OC[C@]56CCCN5[C@H](COC(=O)N5CCN(c7cc([C@@H](C(=O)N8C[C@H](O)C[C@H]8C(=O)N[C@@H](C)c8ccc(C#N)cc8)C(C)C)on7)CC5)CC6)nc4c3F)c12] | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2S)-1- [(2S,4R)-2-{[(1S)-1-(4- cyanophenyl)ethyl] carbamoyl}- 4-hydroxypyrrolidin- 1-yl]-3-methyl-1- oxobutan-2-yl]-1,2-oxazol- |
| | | 3-yl}piperazine-1- |
| | | carboxylate |
| 68 | 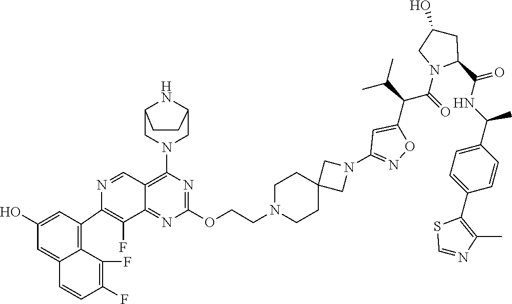 [SMILES: Cc1ncsc1-c1ccc([C@H](C)NC(=O)[C@@H]2C[C@@H](O)CN2C(=O)[C@@H](c2cc(N3CC4(CCN(CCOc5nc(N6CC7CCC(C6)N7)c6cnc(-c7cc(O)cc8ccc(F)c(F)c78)c(F)c6n5)CC4)C3)no2)C(C)C)cc1] | (2S,4R)-1-[(2R*)-2-[3-(7- {2-[(4-{3,8- diazabicyclo[3.2.1]octan-3- yl}-7-(7,8-difluoro-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(4- methyl-1,3-thiazol-5- yl)phenyl]ethyl]pyrrolidine- 2-carboxamide |
| 69 | 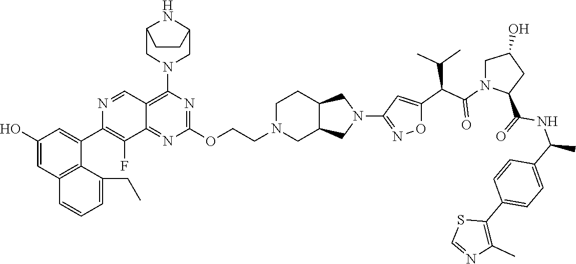 [SMILES: CCc1cccc2cc(O)cc(-c3ncc4c(N5CC6CCC(C5)N6)nc(OCCN5CC[C@H]6CN(c7cc([C@H](C(=O)N8C[C@H](O)C[C@H]8C(=O)N[C@@H](C)c8ccc(-c9scnc9C)cc8)C(C)C)on7)C[C@H]6C5)nc4c3F)c12] | (2S,4R)-1-[(2R*)-2-{3- [(3aR**,7aR***)-5-{2-[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-octahydro- 1H-pyrrolo[3,4-c]pyridin- 2-yl]-1,2-oxazol-5-yl}-3- methylbutanoyl]-4- hydroxy-N-[(1S)-1-[4-(4- methyl-1,3-thiazol-5- yl)phenyl]ethyl]pyrrolidine- 2-carboxamide |
| 70 | 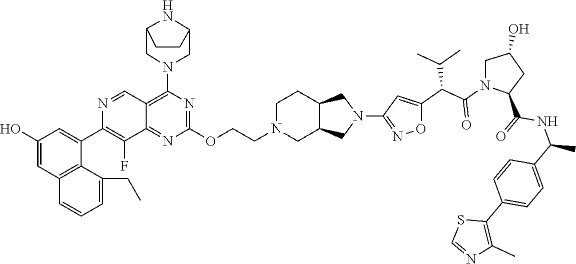 [SMILES: CCc1cccc2cc(O)cc(-c3ncc4c(N5CC6CCC(C5)N6)nc(OCCN5CC[C@H]6CN(c7cc([C@@H](C(=O)N8C[C@H](O)C[C@H]8C(=O)N[C@@H](C)c8ccc(-c9scnc9C)cc8)C(C)C)on7)C[C@H]6C5)nc4c3F)c12] | (2S,4R)-1-[(2R*)-2-{3- [(3aR**,7aR***)-5-{2-[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-octahydro- 1H-pyrrolo[3,4-c]pyridin- 2-yl]-1,2-oxazol-5-yl}-3- methylbutanoyl]-4- hydroxy-N-[(1S)-1-[4-(4- methyl-1,3-thiazol-5- yl)phenyl]ethyl]pyrrolidine- 2-carboxamide |
| 71 | 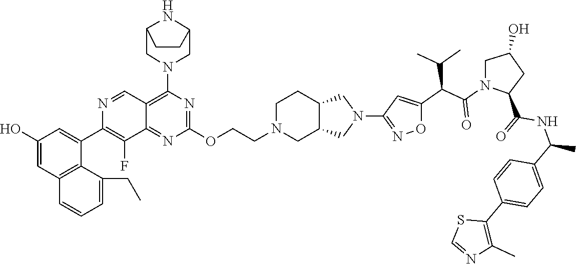 [SMILES: CCc1cccc2cc(O)cc(-c3ncc4c(N5CC6CCC(C5)N6)nc(OCCN5CC[C@@H]6CN(c7cc([C@H](C(=O)N8C[C@H](O)C[C@H]8C(=O)N[C@@H](C)c8ccc(-c9scnc9C)cc8)C(C)C)on7)C[C@@H]6C5)nc4c3F)c12] | (2S,4R)-1-[(2R*)-2-{3- [(3aR**,7aR***)-5-{2-[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-octahydro- 1H-pyrrolo[3,4-c]pyridin- 2-yl]-1,2-oxazol-5-yl}-3- methylbutanoyl]-4- hydroxy-N-[(1S)-1-[4-(4- methyl-1,3-thiazol-5- yl)phenyl]ethyl]pyrrolidine- 2-carboxamide |
| 72 | 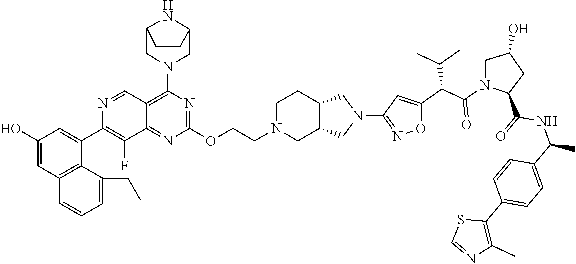 [SMILES: CCc1cccc2cc(O)cc(-c3ncc4c(N5CC6CCC(C5)N6)nc(OCCN5CC[C@@H]6CN(c7cc([C@@H](C(=O)N8C[C@H](O)C[C@H]8C(=O)N[C@@H](C)c8ccc(-c9scnc9C)cc8)C(C)C)on7)C[C@@H]6C5)nc4c3F)c12] | (2S,4R)-1-[(2R*)-2-{3- [(3aR**,7aR***)-5-{2-[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-octahydro- 1H-pyrrolo[3,4-c]pyridin- 2-yl]-1,2-oxazol-5-yl}-3- methylbutanoyl]-4- hydroxy-N-[(1S)-1-[4-(4- methyl-1,3-thiazol-5- yl)phenyl]ethyl]pyrrolidine- 2-carboxamide |
| 73 | 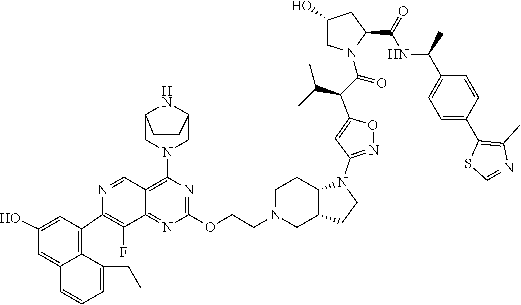 [SMILES: CCc1cccc2cc(O)cc(-c3ncc4c(N5CC6CCC(C5)N6)nc(OCCN5CC[C@H]6[C@H](CCN6c6cc([C@H](C(=O)N7C[C@H](O)C[C@H]7C(=O)N[C@@H](C)c7ccc(-c8scnc8C)cc7)C(C)C)on6)C5)nc4c3F)c12] | (2S,4R)-1-[(2R*)-2-{3- [(3aR**,7aR***)-5-{2-[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-octahydro- 1H-pyrrolo[3,2-c]pyridin- 1-yl]-1,2-oxazol-5-yl}-3- methylbutanoyl]-4- hydroxy-N-[(1S)-1-[4-(4- methyl-1,3-thiazol-5- yl)phenyl]ethyl]pyrrolidine- 2-carboxamide |
| 74 | 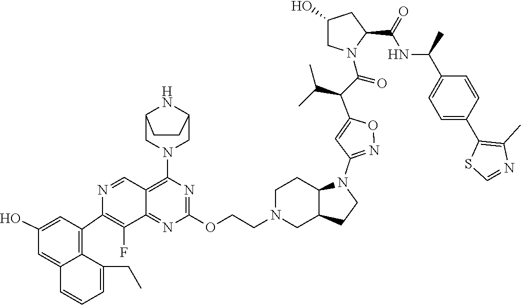 [SMILES: CCc1cccc2cc(O)cc(-c3ncc4c(N5CC6CCC(C5)N6)nc(OCCN5CC[C@@H]6[C@@H](CCN6c6cc([C@H](C(=O)N7C[C@H](O)C[C@H]7C(=O)N[C@@H](C)c7ccc(-c8scnc8C)cc7)C(C)C)on6)C5)nc4c3F)c12] | (2S,4R)-1-[(2R*)-2-{3- [(3aR**,7aR***)-5-{2-[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-octahydro- 1H-pyrrolo[3,2-c]pyridin- 1-yl]-1,2-oxazol-5-yl}-3- methylbutanoyl]-4- hydroxy-N-[(1S)-1-[4-(4- methyl-1,3-thiazol-5- yl)phenyl]ethyl]pyrrolidine- 2-carboxamide |
| 75 | 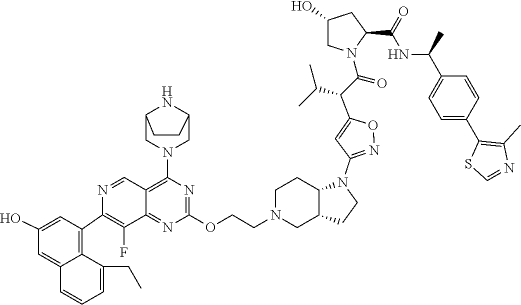 [SMILES: CCc1cccc2cc(O)cc(-c3ncc4c(N5CC6CCC(C5)N6)nc(OCCN5CC[C@H]6[C@H](CCN6c6cc([C@@H](C(=O)N7C[C@H](O)C[C@H]7C(=O)N[C@@H](C)c7ccc(-c8scnc8C)cc7)C(C)C)on6)C5)nc4c3F)c12] | (2S,4R)-1-[(2R*)-2-{3- [(3aR**,7aR***)-5-{2-[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-octahydro- 1H-pyrrolo[3,2-c]pyridin- 1-yl]-1,2-oxazol-5-yl}-3- methylbutanoyl]-4- hydroxy-N-[(1S)-1-[4-(4- methyl-1,3-thiazol-5- yl)phenyl]ethyl]pyrrolidine- 2-carboxamide |
| 76 | 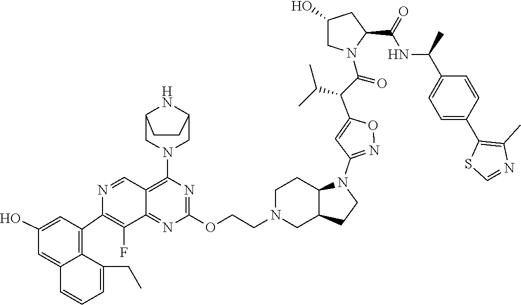 [SMILES: CCc1cccc2cc(O)cc(-c3ncc4c(N5CC6CCC(C5)N6)nc(OCCN5CC[C@@H]6[C@@H](CCN6c6cc([C@@H](C(=O)N7C[C@H](O)C[C@H]7C(=O)N[C@@H](C)c7ccc(-c8scnc8C)cc7)C(C)C)on6)C5)nc4c3F)c12] | (2S,4R)-1-[(2R*)-2-{3- [(3aR**,7aR***)-5-{2-[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-octahydro- 1H-pyrrolo[3,2-c]pyridin- 1-yl]-1,2-oxazol-5-yl}-3- methylbutanoyl]-4- hydroxy-N-[(1S)-1-[4-(4- methyl-1,3-thiazol-5- yl)phenyl]ethyl]pyrrolidine- 2-carboxamide |
| 77 | 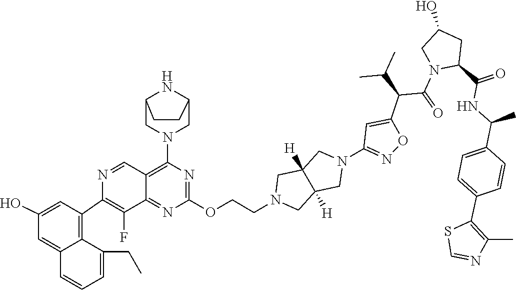 [SMILES: CCc1cccc2cc(O)cc(-c3ncc4c(N5CC6CCC(C5)N6)nc(OCCN5C[C@@H]6CN(c7cc([C@H](C(=O)N8C[C@H](O)C[C@H]8C(=O)N[C@@H](C)c8ccc(-c9scnc9C)cc8)C(C)C)on7)C[C@H]6C5)nc4c3F)c12] | (2S,4R)-1-[(2R*)-2-{3- [(3aR,6aR)-5-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}- octahydropyrrolo[3,4- c]pyrrol-2-yl]-1,2-oxazol- 5-yl}-3-methylbutanoyl]-4- hydroxy-N-[(1S)-1-[4-(4- methyl-1,3-thiazol-5- yl)phenyl]ethyl]pyrrolidine- 2-carboxamide |
| 78 | 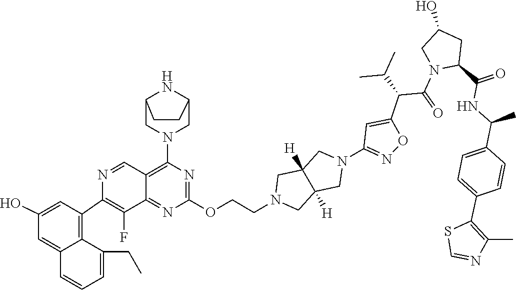 [SMILES: CCc1cccc2cc(O)cc(-c3ncc4c(N5CC6CCC(C5)N6)nc(OCCN5C[C@@H]6CN(c7cc([C@@H](C(=O)N8C[C@H](O)C[C@H]8C(=O)N[C@@H](C)c8ccc(-c9scnc9C)cc8)C(C)C)on7)C[C@H]6C5)nc4c3F)c12] | (2S,4R)-1-[(2R*)-2-{3- [(3aR,6aR)-5-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}- octahydropyrrolo[3,4- c]pyrrol-2-yl]-1,2-oxazol- 5-yl}-3-methylbutanoyl]-4- hydroxy-N-[(1S)-1-[4-(4- methyl-1,3-thiazol-5- yl)phenyl]ethyl]pyrrolidine- 2-carboxamide |
| 79 | 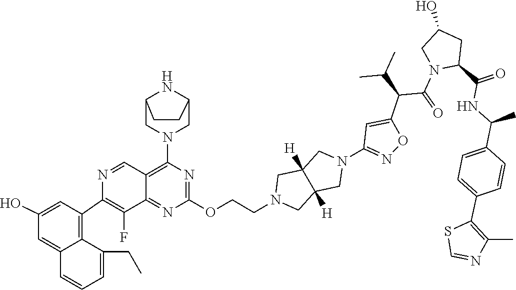 [SMILES: CCc1cccc2cc(O)cc(-c3ncc4c(N5CC6CCC(C5)N6)nc(OCCN5C[C@@H]6CN(c7cc([C@H](C(=O)N8C[C@H](O)C[C@H]8C(=O)N[C@@H](C)c8ccc(-c9scnc9C)cc8)C(C)C)on7)C[C@@H]6C5)nc4c3F)c12] | (2S,4R)-1-[(2R*)-2-{3- [(3aR,6aS)-5-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}- octahydropyrrolo[3,4- c]pyrrol-2-yl]-1,2-oxazol- 5-yl}-3-methylbutanoyl]-4- hydroxy-N-[(1S)-1-[4-(4- methyl-1,3-thiazol-5- yl)phenyl]ethyl]pyrrolidine- 2-carboxamide |
| 80 | 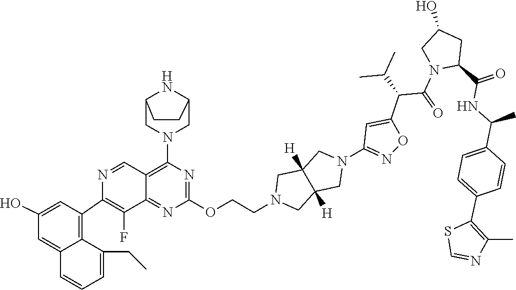 [SMILES: CCc1cccc2cc(O)cc(-c3ncc4c(N5CC6CCC(C5)N6)nc(OCCN5C[C@@H]6CN(c7cc([C@@H](C(=O)N8C[C@H](O)C[C@H]8C(=O)N[C@@H](C)c8ccc(-c9scnc9C)cc8)C(C)C)on7)C[C@@H]6C5)nc4c3F)c12] | (2S,4R)-1-[(2R*)-2-{3- [(3aR,6aS)-5-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}- octahydropyrrolo[3,4- c]pyrrol-2-yl]-1,2-oxazol- 5-yl}-3-methylbutanoyl]-4- hydroxy-N-[(1S)-1-[4-(4- methyl-1,3-thiazol-5- yl)phenyl]ethyl]pyrrolidine- 2-carboxamide |
| 81 | 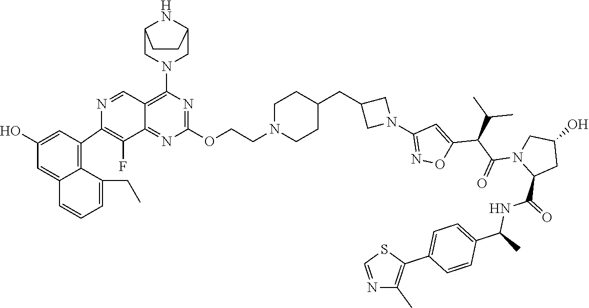 [SMILES: CCc1cccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-(3-{3- [(1-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl]pyrrolidine- 2-carboxamide |
| 82 | 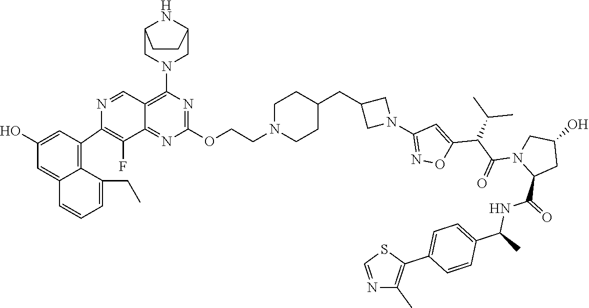 | (2S,4R)-1-[(2R*)-2-(3-{3- [(1-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl]pyrrolidine- 2-carboxamide |
| 83 | 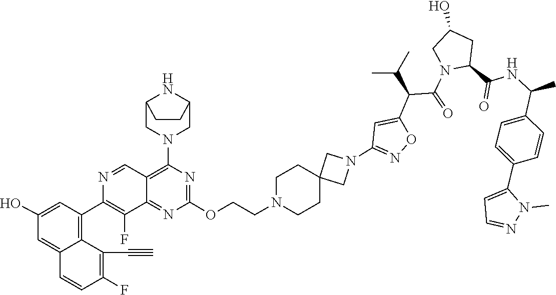 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8ccnn8C)cc7)C(C)C)on5)C6)nc4c3F)c12] | (2S,4R)-1-[(2R*)-2-[3-(7- {2-[(4-{3,8- diazabicyclo[3.2.1]octan-3- yl}-7-(8-ethynyl-7-fluoro- 3-hydroxynaphthalen-1- yl)-8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(1- methyl-1H-pyrazol-5- yl)phenyl]ethyl]pyrrolidine- 2-carboxamide |
| 84 | 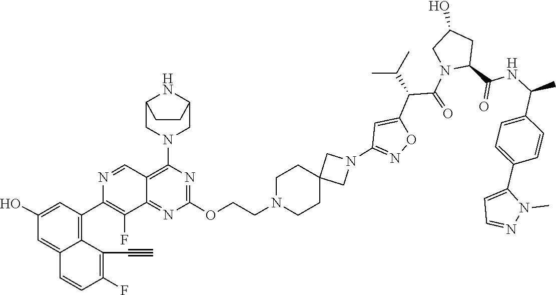 [SMILES: C#Cc1c(F)ccc2cc(O)cc(-c3ncc4c(N5CC6CCC(C5)N6)nc(OCCN5CCC6(CC5)CN(c5cc([C@@H](C(=O)N7C[C@H](O)C[C@H]7C(=O)N[C@@H](C)c7ccc(-c8ccnn8C)cc7)C(C)C)on5)C6)nc4c3F)c12] | (2S,4R)-1-[(2S)-2-[3-[7-[2- [4-(3,8-diazabicyclo [3.2.1]octan-3-yl)-7-(8- ethynyl-7-fluoro-3- hydroxy-1-naphthyl)-8- fluoro-pyrido[4,3- d]pyrimidin-2- yl]oxyethyl]-2,7- diazaspiro[3.5]nonan-2- yl]isoxazol-5-yl]-3-methyl- butanoyl]-4-hydroxy-N- [(1S)-1-[4-(2- methylpyrazol-3- yl)phenyl]ethyl]pyrrolidine- 2-carboxamide |
| 85 | 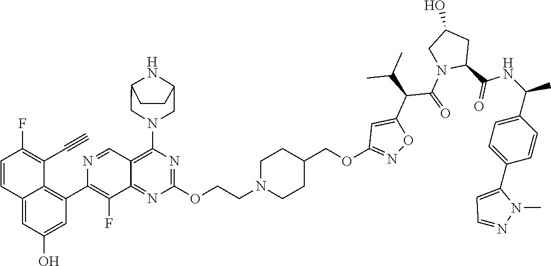 [SMILES: C#Cc1c(F)ccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8ccnn8C)cc7)C(C)C)on6)CC5)nc4c3F)c12] | (2S,4R)-1-[(2R*)-2-{3-[(1- {2-[(4-{3,8- diazabicyclo[3.2.1]octan-3- yl}-7-(8-ethynyl-7-fluoro- 3-hydroxynaphthalen-1- yl)-8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(1- methyl-1H-pyrazol-5- yl)phenyl]ethyl]pyrrolidine- 2-carboxamide |
| 86 | 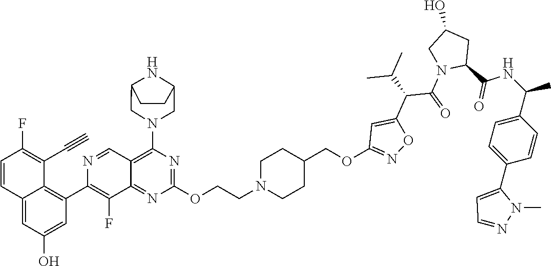 [SMILES: C#Cc1c(F)ccc2cc(O)cc(-c3ncc4c(N5CC6CCC(C5)N6)nc(OCCN5CCC(COc6cc([C@@H](C(=O)N7C[C@H](O)C[C@H]7C(=O)N[C@@H](C)c7ccc(-c8ccnn8C)cc7)C(C)C)on6)CC5)nc4c3F)c12] | (2S,4R)-1-[(2R*)-2-{3-[(1- {2-[(4-{3,8- diazabicyclo[3.2.1]octan-3- yl}-7-(8-ethynyl-7-fluoro- 3-hydroxynaphthalen-1- yl)-8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(1- methyl-1H-pyrazol-5- yl)phenyl]ethyl]pyrrolidine- 2-carboxamide |
| 87 | 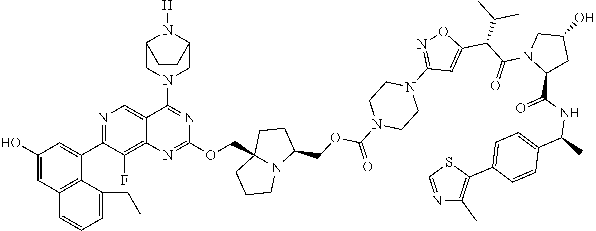 [SMILES: CCc1cccc2cc(O)cc(-c3ncc4c(N5CC6CCC(C5)N6)nc(OC[C@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 88 | 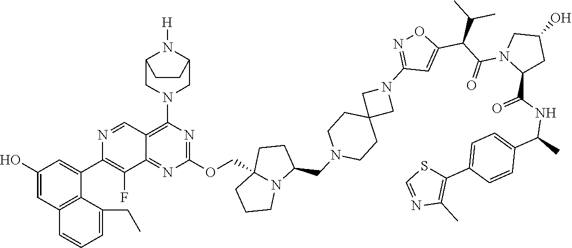 [SMILES: CCc1cccc2cc(O)cc(-c3ncc4c(N5CC6CCC(C5)N6)nc(OC[C@@]56CCCN5[C@H](CN5CCC7(CC5)CN(c5cc([C@H](C(=O)N8C[C@H](O)C[C@H]8C(=O)N[C@@H](C)c8ccc(-c9scnc9C)cc8)C(C)C)on5)C7)CC6)nc4c3F)c12] | (2S,4R)-1-[(2R)-2-[3-(7- {[(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl}-2,7- diazaspiro[3.5]nonan-2- yl)-1,2-oxazol-5-yl]-3- methylbutanoyl]-4- hydroxy-N-[(1S)-1-[4-(4- methyl-1,3-thiazol-5- yl)phenyl]ethyl]pyrrolidine- |
| | | 2-carboxamide |
| 89 | 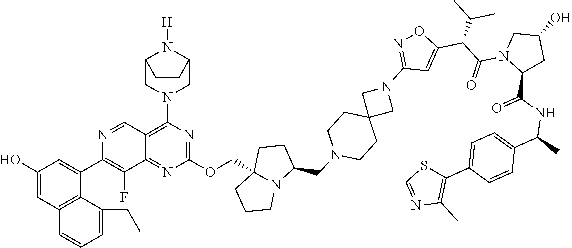 [SMILES: CCc1cccc2cc(O)cc(-c3ncc4c(N5CC6CCC(C5)N6)nc(OC[C@@]56CCCN5[C@H](CN5CCC7(CC5)CN(c5cc([C@@H](C(=O)N8C[C@H](O)C[C@H]8C(=O)N[C@@H](C)c8ccc(-c9scnc9C)cc8)C(C)C)on5)C7)CC6)nc4c3F)c12] | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}methyl)-N- |
| | | methylcarbamate |
| 90 | 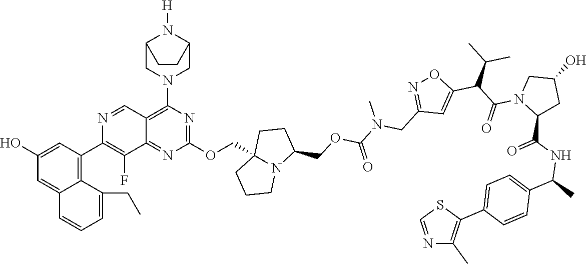 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}methyl)-N- methylcarbamate |
| 91 | 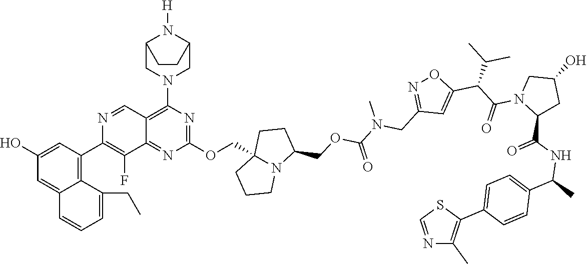 [SMILES: CCc1cccc2cc(O)cc(-c3ncc4c(N5CC6CCC(C5)N6)nc(OC[C@@]56CCCN5[C@H](COC(=O)N(C)Cc5cc([C@@H](C(=O)N7C[C@H](O)C[C@H]7C(=O)N[C@@H](C)c7ccc(-c8scnc8C)cc7)C(C)C)on5)CC6)nc4c3F)c12] | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}methyl)-N- methylcarbamate |
| 92 | 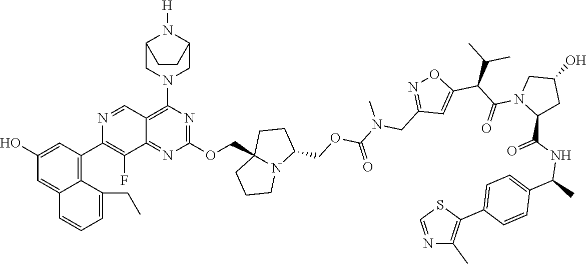 [SMILES: CCc1cccc2cc(O)cc(-c3ncc4c(N5CC6CCC(C5)N6)nc(OC[C@]56CCCN5[C@@H](COC(=O)N(C)Cc5cc([C@H](C(=O)N7C[C@H](O)C[C@H]7C(=O)N[C@@H](C)c7ccc(-c8scnc8C)cc7)C(C)C)on5)CC6)nc4c3F)c12] | [(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}methyl)-N- methylcarbamate |
| 93 | 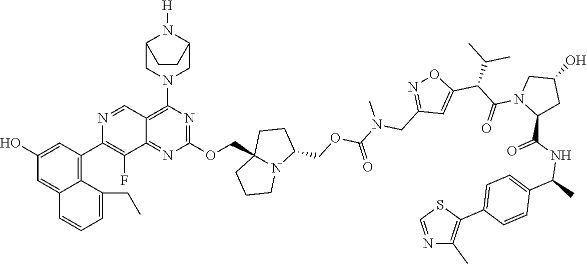 [SMILES: CCc1cccc2cc(O)cc(-c3ncc4c(N5CC6CCC(C5)N6)nc(OC[C@]56CCCN5[C@@H](COC(=O)N(C)Cc5cc([C@@H](C(=O)N7C[C@H](O)C[C@H]7C(=O)N[C@@H](C)c7ccc(-c8scnc8C)cc7)C(C)C)on5)CC6)nc4c3F)c12] | [(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}methyl)-N- methylcarbamate |
| 94 | 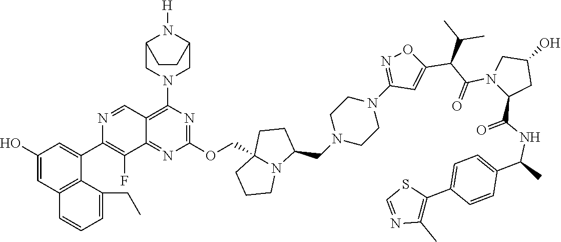 [SMILES: CCc1cccc2cc(O)cc(-c3ncc4c(N5CC6CCC(C5)N6)nc(OC[C@@]56CCCN5[C@H](CN5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | (2S,4R)-1-[(2R)-2-[3-(4- {[(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl}piperazin-1- yl)-1,2-oxazol-5-yl]-3- methylbutanoyl]-4- hydroxy-N-[(1S)-1-[4-(4- methyl-1,3-thiazol-5- yl)phenyl]ethyl]pyrrolidine- 2-carboxamide |
| 95 | 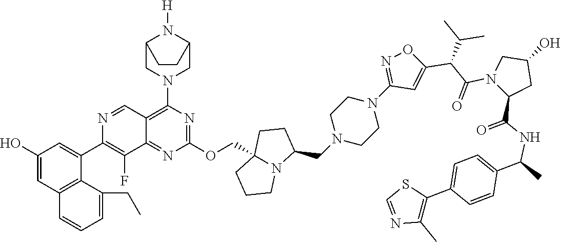 [SMILES: CCc1cccc2cc(O)cc(-c3ncc4c(N5CC6CCC(C5)N6)nc(OC[C@@]56CCCN5[C@H](CN5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12] | (2S,4R)-1-[(2S)-2-[3-(4- {[(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl}piperazin-1- yl)-1,2-oxazol-5-yl]-3- methylbutanoyl]-4- hydroxy-N-[(1S)-1-[4-(4- methyl-1,3-thiazol-5- yl)phenyl]ethyl]pyrrolidine- 2-carboxamide |
| 96 | 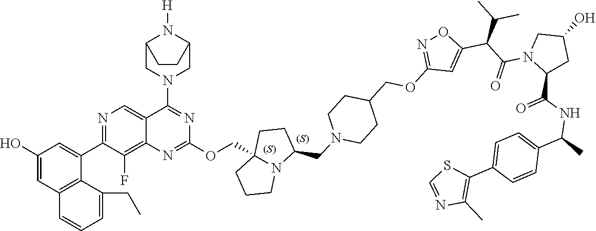 | (2S,4R)-1-[(2R)-2-{3-[(1- {[(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- |
| | | 2-carboxamide |
| 97 | 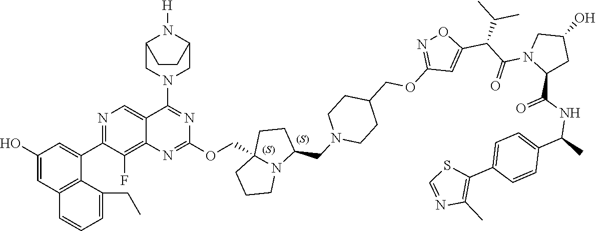 | (2S,4R)-1-[(2S)-2-{3-[(1- {[(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- |
| | | 2-carboxamide |
| 98 | 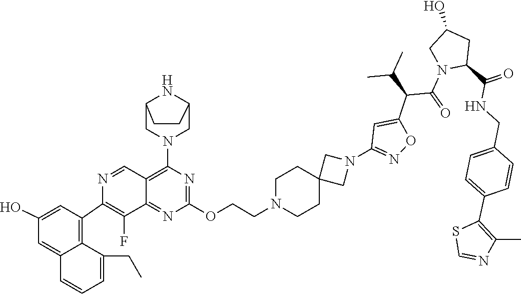 [SMILES: CCc1cccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)NCc7ccc(-c8scnc8C)cc7)C(C)C)on5)C6)nc4c3F)c12] | (2S,4R)-1-[(2R*)-2-[3-(7- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-{[4-(4-methyl- 1,3-thiazol-5- yl)phenyl]methyl} pyrrolidine- 2-carboxamide |
| 99 | 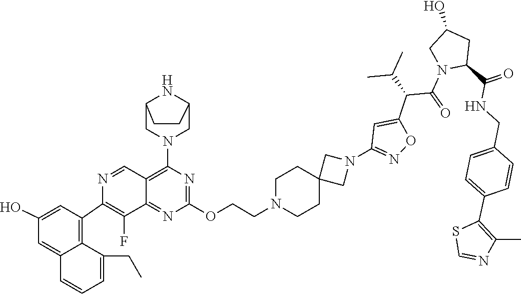 [SMILES: CCc1cccc2cc(O)cc(-c3ncc4c(N5CC6CCC(C5)N6)nc(OCCN5CCC6(CC5)CN(c5cc([C@@H](C(=O)N7C[C@H](O)C[C@H]7C(=O)NCc7ccc(-c8scnc8C)cc7)C(C)C)on5)C6)nc4c3F)c12] | (2S,4R)-1-[(2R*)-2-[3-(7- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-{[4-(4-methyl- 1,3-thiazol-5- yl)phenyl]methyl} pyrrolidine- 2-carboxamide |
| 100 | 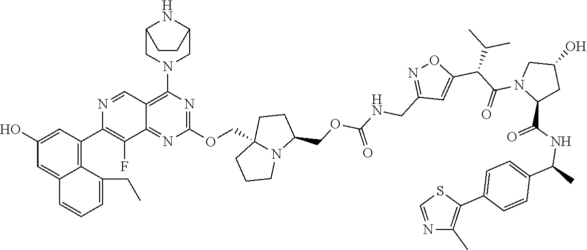 [SMILES: CCc1cccc2cc(O)cc(-c3ncc4c(N5CC6CCC(C5)N6)nc(OC[C@@]56CCCN5[C@H](COC(=O)NCc5cc([C@@H](C(=O)N7C[C@H](O)C[C@H]7C(=O)N[C@@H](C)c7ccc(-c8scnc8C)cc7)C(C)C)on5)CC6)nc4c3F)c12] | [(3S,7aS)-7a-{[(4-{3,8- diazabicyclo [3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2S)-1- [(2S,4R)-4-hydroxy-2- {[(1S)-1-[4-(4-methyl-1,3- thiazol-5- yl)phenyl]ethyl] carbamoyl} pyrrolidin-1-yl]-3-methyl- |
| | | 1-oxobutan-2-yl]-1,2- |
| | | oxazol-3- |
| | | yl}methyl)carbamate |
| 101 | 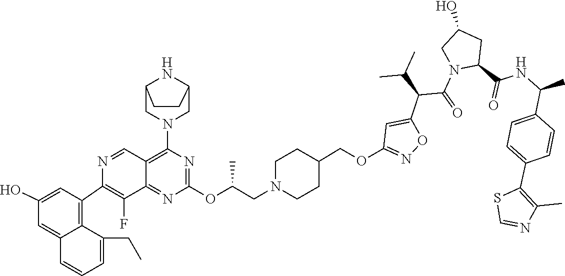 [SMILES: CCc1cccc2cc(O)cc(-c3ncc4c(N5CC6CCC(C5)N6)nc(O[C@H](C)CN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-[3-({1- [(2R)-2-[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]propyl]piperidin-4- yl}methoxy)-1,2-oxazol-5- yl]-3-methylbutanoyl]-4- hydroxy-N-[(1S)-1-[4-(4- methyl-1,3-thiazol-5- yl)phenyl]ethyl] pyrrolidine- 2-carboxamide |
| 102 | 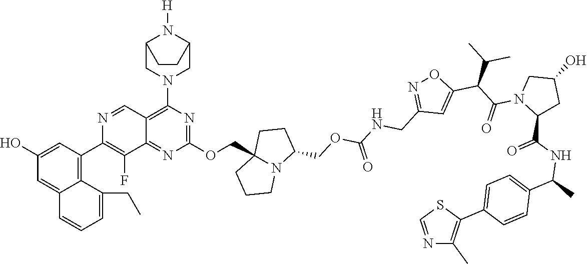 [SMILES: CCc1cccc2cc(O)cc(-c3ncc4c(N5CC6CCC(C5)N6)nc(OC[C@]56CCCN5[C@@H](COC(=O)NCc5cc([C@H](C(=O)N7C[C@H](O)C[C@H]7C(=O)N[C@@H](C)c7ccc(-c8scnc8C)cc7)C(C)C)on5)CC6)nc4c3F)c12] | [(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3- yl}methyl)carbamate |
| 103 | 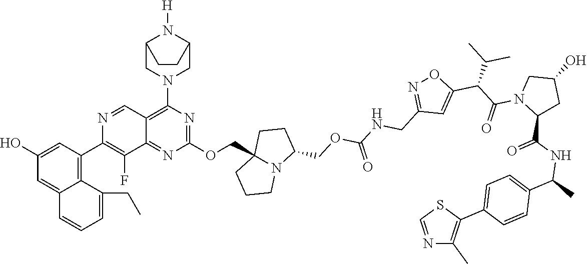 [SMILES: CCc1cccc2cc(O)cc(-c3ncc4c(N5CC6CCC(C5)N6)nc(OC[C@]56CCCN5[C@@H](COC(=O)NCc5cc([C@@H](C(=O)N7C[C@H](O)C[C@H]7C(=O)N[C@@H](C)c7ccc(-c8scnc8C)cc7)C(C)C)on5)CC6)nc4c3F)c12] | [(3R,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3- yl}methyl)carbamate |
| 104 | 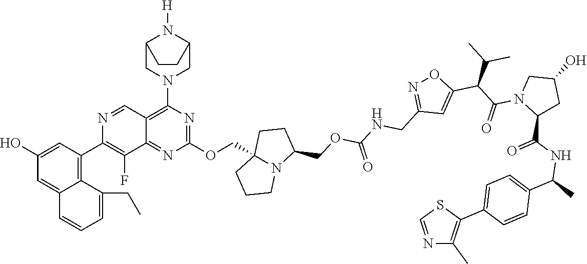 [SMILES: CCc1cccc2cc(O)cc(-c3ncc4c(N5CC6CCC(C5)N6)nc(OC[C@@]56CCCN5[C@H](COC(=O)NCc5cc([C@H](C(=O)N7C[C@H](O)C[C@H]7C(=O)N[C@@H](C)c7ccc(-c8scnc8C)cc7)C(C)C)on5)CC6)nc4c3F)c12] | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl N-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3- yl}methyl)carbamate |
| 105 | 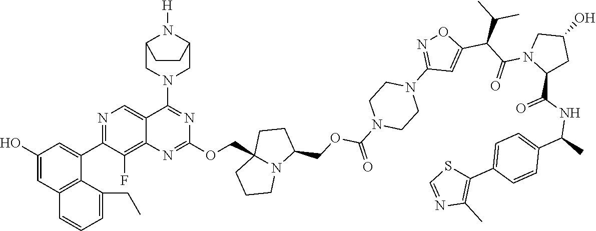 [SMILES: CCc1cccc2cc(O)cc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 106 | 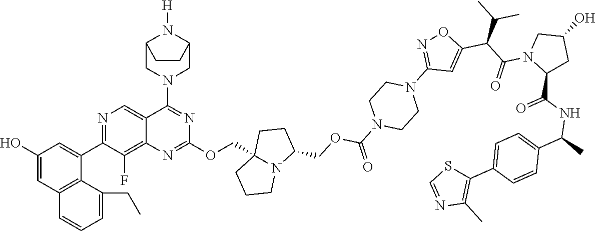 [SMILES: CCc1cccc2cc(O)cc(-c3ncc4c(N5CC6CCC(C5)N6)nc(OC[C@@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 107 | 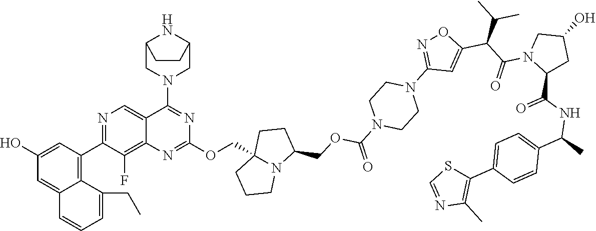 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R*,7aR**)-7a-{[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R***)- 1-[(2S,4R)-4-hydroxy-2- {[(1S)-1-[4-(4-methyl-1,3- thiazol-5- yl)phenyl]ethyl]carbamoyl} pyrrolidin-1-yl]-3-methyl- |
| | | 1-oxobutan-2-yl]-1,2- |
| | | oxazol-3-yl}piperazine-1- |
| | | carboxylate |
| 108 | 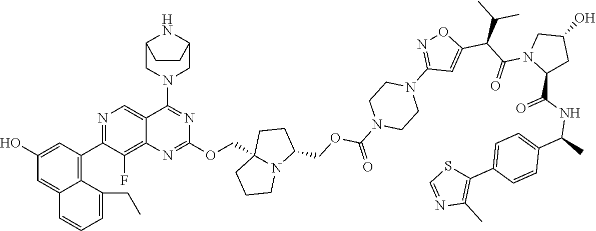 [SMILES: CCc1cccc2cc(O)cc(-c3ncc4c(N5CC6CCC(C5)N6)nc(OC[C@@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 109 | 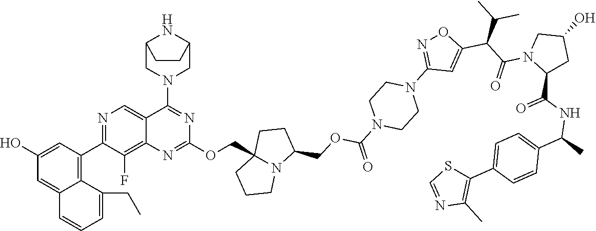 [SMILES: CCc1cccc2cc(O)cc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}piperazine-1- |
| | | carboxylate |
| 110 | 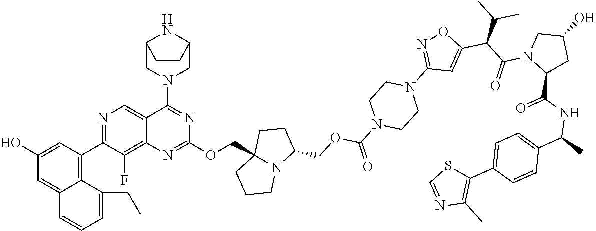 [SMILES: CCc1cccc2cc(O)cc(-c3ncc4c(N5CC6CCC(C5)N6)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R*,7aR**)-7a-{[(4- {3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-{5-[(2R***)- 1-[(2S,4R)-4-hydroxy-2- {[(1S)-1-[4-(4-methyl-1,3- thiazol-5- yl)phenyl]ethyl]carbamoyl} pyrrolidin-1-yl]-3-methyl- |
| | | 1-oxobutan-2-yl]-1,2- |
| | | oxazol-3-yl}piperazine-1- |
| | | carboxylate |
| 111 | 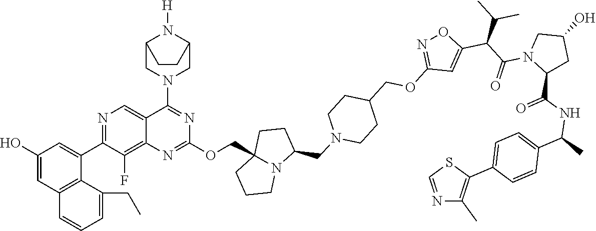 [SMILES: CCc1cccc2cc(O)cc(-c3ncc4c(N5CC6CCC(C5)N6)nc(OC[C@]56CCCN5[C@H](CN5CCC(COc7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | (2S,4R)-1-[(2R)-2-{3-[(1- {[(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl}piperidin-4- yl)methoxy]-1,2-oxazol-5- yl}-3-methylbutanoyl]-4- hydroxy-N-[(1S)-1-[4-(4- methyl-1,3-thiazol-5- yl)phenyl]ethyl]pyrrolidine- |
| | | 2-carboxamide |
| 112 | 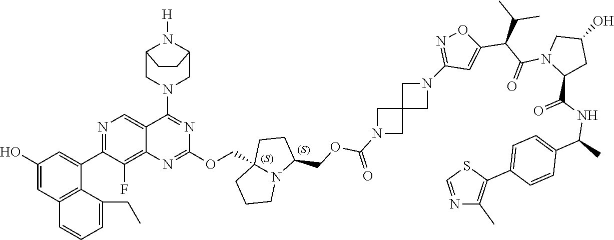 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 6-{5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}-2,6- |
| | | diazaspiro[3.3]heptane-2- |
| | | carboxylate |
| 113 | 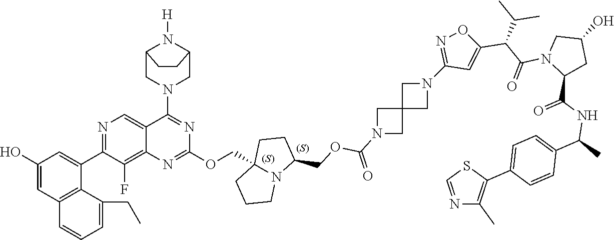 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 6-{5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3-yl}-2,6- |
| | | diazaspiro[3.3]heptane-2- |
| | | carboxylate |
| 114 | 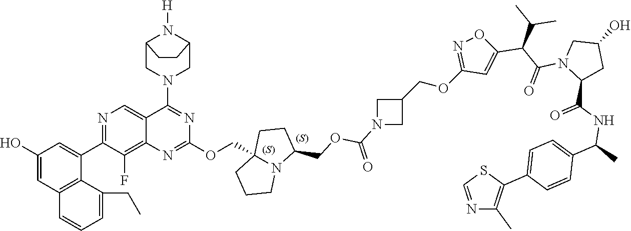 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 3-[({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3- |
| | | yl}oxy)methyl]azetidine-1- |
| | | carboxylate |
| 115 | 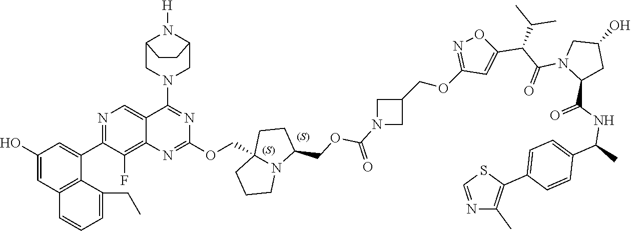 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 3-[({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3- |
| | | yl}oxy)methyl]azetidine-1- |
| | | carboxylate |
| 116 | 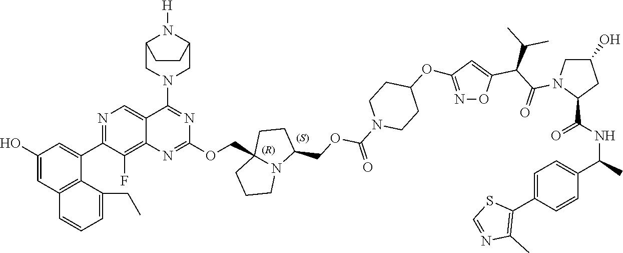 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3- |
| | | yl}oxy)piperidine-1- |
| | | carboxylate |
| 117 | 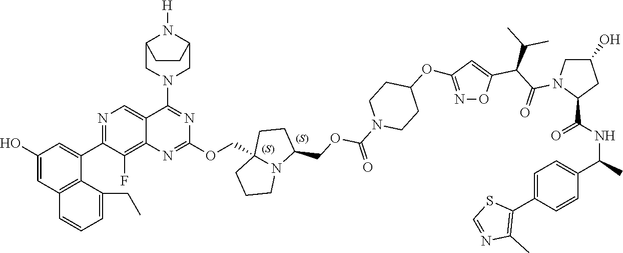 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3- |
| | | yl}oxy)piperidine-1- |
| | | carboxylate |
| 118 | 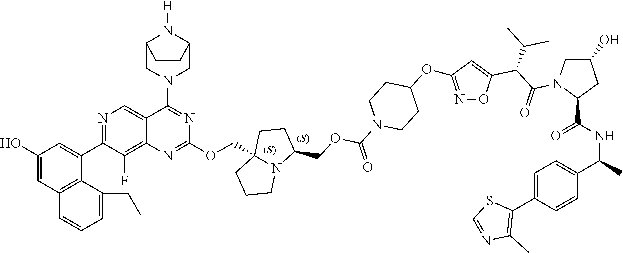 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3- |
| | | yl}oxy)piperidine-1- |
| | | carboxylate |
| 119 | 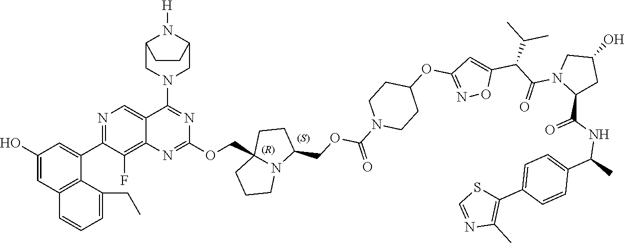 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 4-({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3- |
| | | yl}oxy)piperidine-1- |
| | | carboxylate |
| 120 | 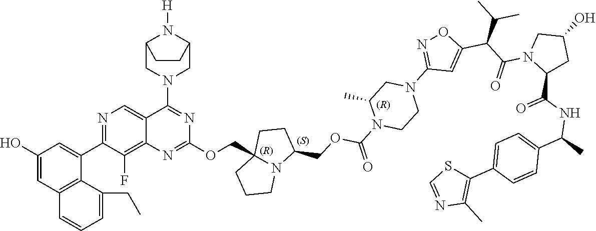 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl- 1-oxobutan-2-yl]-1,2- |
| | | oxazol-3-yl}-2- |
| | | methylpiperazine-1- |
| | | carboxylate |
| 121 | 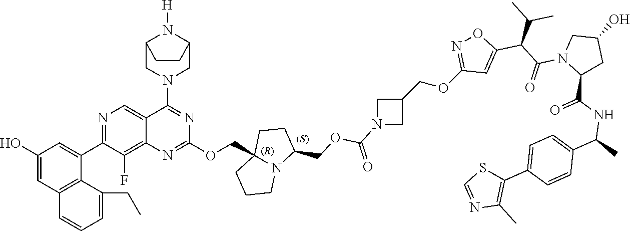 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 3-[({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3- |
| | | yl}oxy)methyl]azetidine-1- |
| | | carboxylate |
| 122 | 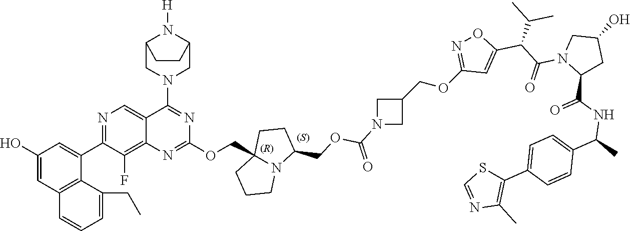 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 3-[({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- |
| | | oxazol-3- |
| | | yl}oxy)methyl]azetidine-1- |
| | | carboxylate |
| 123 | 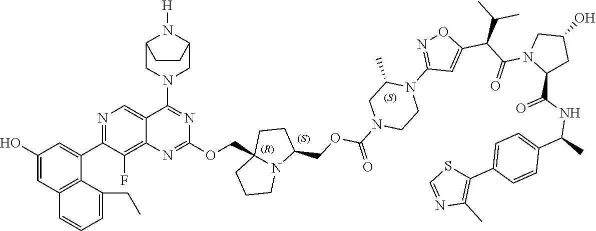 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (3S)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl- 1-oxobutan-2-yl]-1,2- |
| | | oxazol-3-yl}-3- |
| | | methylpiperazine-1- |
| | | carboxylate |
| 124 | 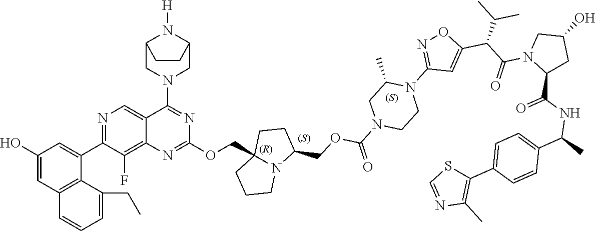 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (3S)-4-{5- [(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl- 1-oxobutan-2-yl]-1,2- |
| | | oxazol-3-yl}-3- |
| | | methylpiperazine-1- |
| | | carboxylate |
| 125 | 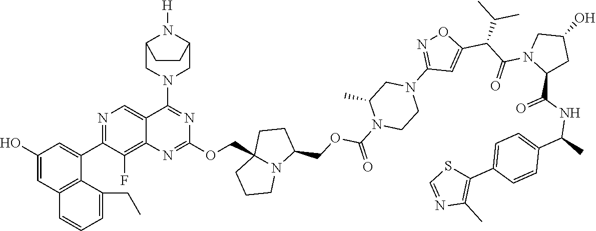 [SMILES: CCc1cccc2cc(O)cc(-c3ncc4c(N5CC6CCC(C5)N6)nc(OC[C@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)C[C@H]5C)CC6)nc4c3F)c12] | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2R)-4-{5- [(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl- 1-oxobutan-2-yl]-1,2- |
| | | oxazol-3-yl}-2- |
| | | methylpiperazine-1- |
| | | carboxylate |
| 126 | 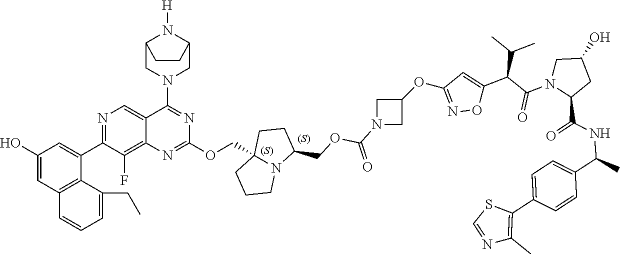 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 3-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}oxy)azetidine- |
| | | 1-carboxylate |
| 127 | 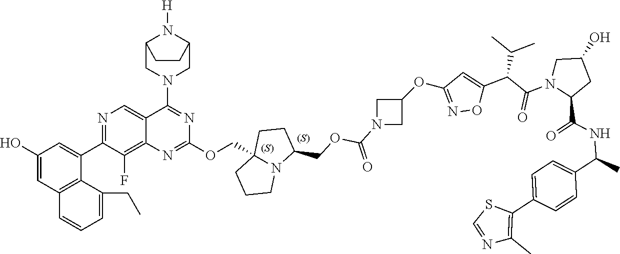 | [(3S,7aS)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 3-({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}oxy)azetidine- |
| | | 1-carboxylate |
| 128 | 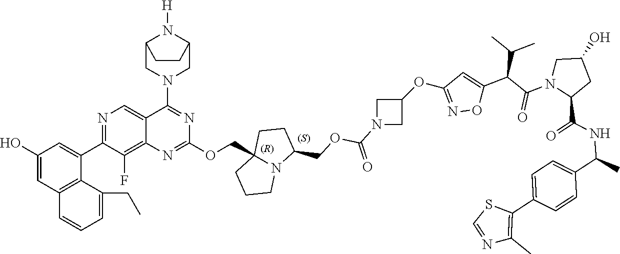 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 3-({5-[(2R)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}oxy)azetidine- |
| | | 1-carboxylate |
| 129 | 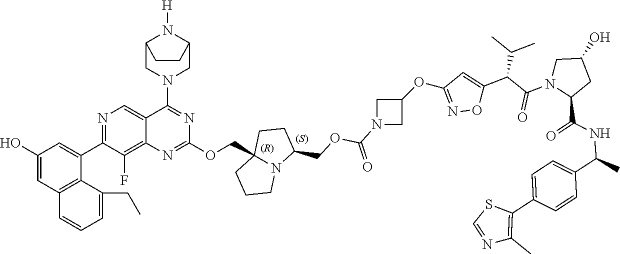 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 3-({5-[(2S)-1- [(2S,4R)-4-hydroxy-2- {[(1S)-1-[4-(4-methyl-1,3- thiazol-5- yl)phenyl]ethyl]carbamoyl} pyrrolidin-1-yl]-3-methyl- 1-oxobutan-2-yl]-1,2- oxazol-3-yl}oxy)azetidine- |
| | | 1-carboxylate |
| 130 | 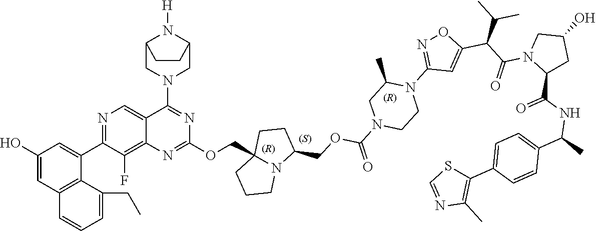 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (3R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl- 1-oxobutan-2-yl]-1,2- |
| | | oxazol-3-yl}-3- |
| | | methylpiperazine-1- |
| | | carboxylate |
| 131 | 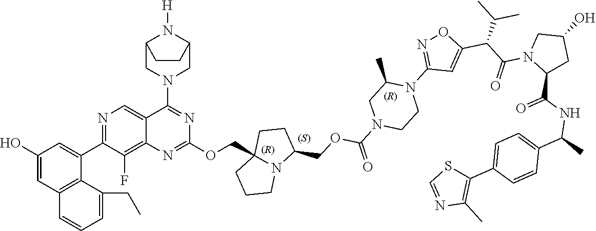 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (3R)-4-{5- [(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl- 1-oxobutan-2-yl]-1,2- |
| | | oxazol-3-yl}-3- |
| | | methylpiperazine-1- |
| | | carboxylate |
| 132 | 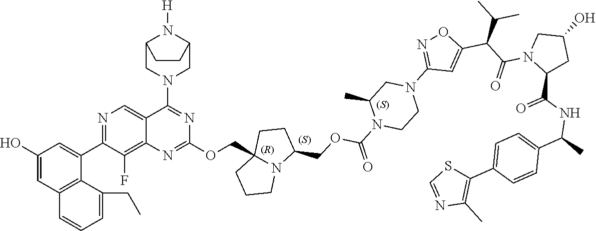 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2S)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl- 1-oxobutan-2-yl]-1,2- |
| | | oxazol-3-yl}-2- |
| | | methylpiperazine-1- |
| | | carboxylate |
| 133 | 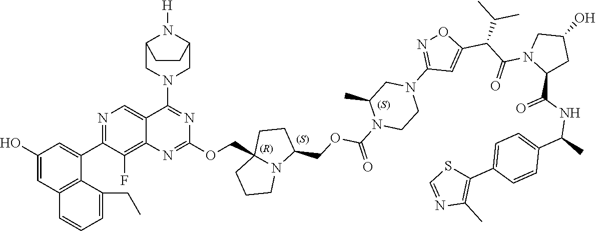 | [(3S,7aR)-7a-{[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2S)-4-{5- [(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl- 1-oxobutan-2-yl]-1,2- |
| | | oxazol-3-yl}-2- |
| | | methylpiperazine-1- |
| | | carboxylate |
| 134 | 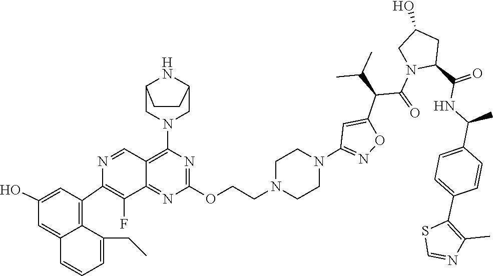 [SMILES: CCc1cccc2cc(O)cc(-c3ncc4c(N5CC6CCC(C5)N6)nc(OCCN5CCN(c6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R*)-2-[3-(4- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperazin-1- yl)-1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 135 | 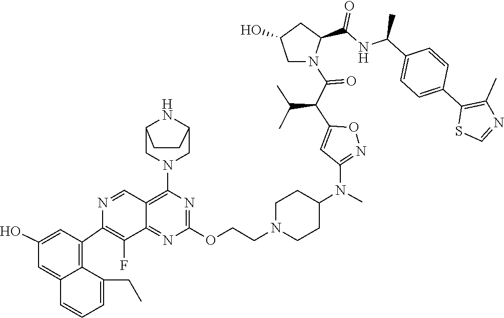 [SMILES: CCc1cccc2cc(O)cc(-c3ncc4c(N5CC6CCC(C5)N6)nc(OCCN5CCC(N(C)c6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R*)-2- {3-[(1-{2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)(methyl)amino]-1,2- oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 136 | 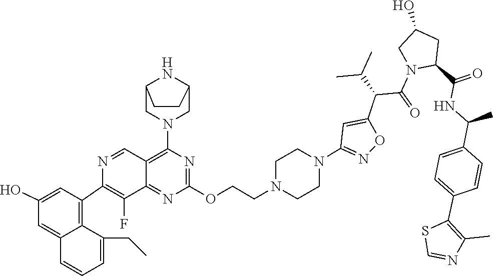 [SMILES: CCc1cccc2cc(O)cc(-c3ncc4c(N5CC6CCC(C5)N6)nc(OCCN5CCN(c6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R*)-2-[3-(4- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperazin-1- yl)-1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 137 | 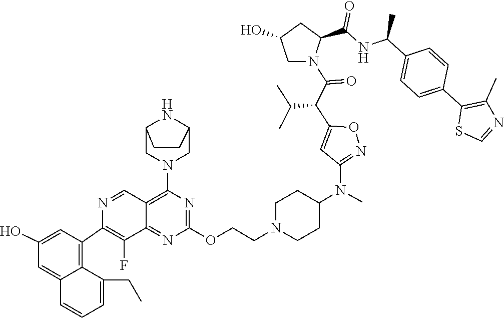 [SMILES: CCc1cccc2cc(O)cc(-c3ncc4c(N5CC6CCC(C5)N6)nc(OCCN5CCC(N(C)c6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R*)-2-{3-[(1- {2-[(4-{3,8- diazabicyclo[3.2.1]octan-3- yl}-7-(8-ethyl-3- hydroxynaphthalen-1-yl)- 8-fluoropyrido[4,3- d]pyrimidin-2- yl)oxy]ethyl}piperidin-4- yl)(methyl)amino]-1,2- oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 138 | 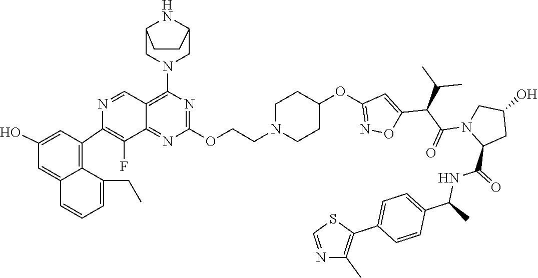 [SMILES: CCc1cccc2cc(O)cc(-c3ncc4c(N5CC6CCC(C5)N6)nc(OCCN5CCC(Oc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R*)-2- {3-[(1-{2-[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]ethyl}piperidin-4- yl)oxy]-1,2-oxazol-5-yl}- 3-methylbutanoyl]-4- hydroxy-N-[(1S)-1-[4-(4- methyl-1,3-thiazol-5- yl)phenyl]ethyl] pyrrolidine- 2-carboxamide |
| 139 | 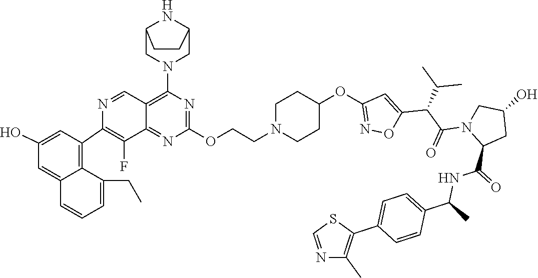 [SMILES: CCc1cccc2cc(O)cc(-c3ncc4c(N5CC6CCC(C5)N6)nc(OCCN5CCC(Oc6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R*)-2- {3-[(1-{2-[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]ethyl}piperidin-4- yl)oxy]-1,2-oxazol-5-yl}- 3-methylbutanoyl]-4- hydroxy-N-[(1S)-1-[4-(4- methyl-1,3-thiazol-5- yl)phenyl]ethyl] pyrrolidine- 2-carboxamide |
| 140 | 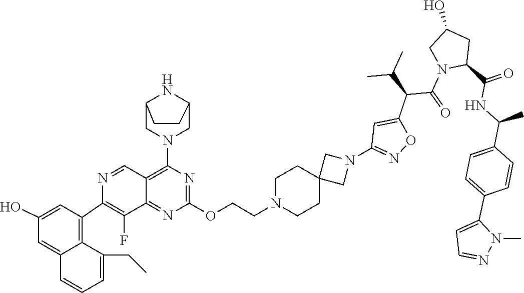 [SMILES: CCc1cccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8ccnn8C)cc7)C(C)C)on5)C6)nc4c3F)c12] | (2S,4R)-1-[(2R*)-2- [3-(7-{2-[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(1- methyl-1H-pyrazol-5- yl)phenyl]ethyl] pyrrolidine- 2-carboxamide |
| 141 | 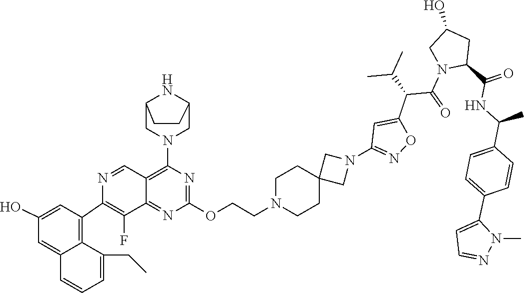 [SMILES: CCc1cccc2cc(O)cc(-c3ncc4c(N5CC6CCC(C5)N6)nc(OCCN5CCC6(CC5)CN(c5cc([C@@H](C(=O)N7C[C@H](O)C[C@H]7C(=O)N[C@@H](C)c7ccc(-c8ccnn8C)cc7)C(C)C)on5)C6)nc4c3F)c12] | (2S,4R)-1-[(2R*)-2- [3-(7-{2-[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]ethyl}-2,7- diazaspiro[3.5]nonan-2- yl)-1,2-oxazol-5-yl]-3- methylbutanoyl]-4- hydroxy-N-[(1S)-1-[4-(1- methyl-1H-pyrazol-5- yl)phenyl]ethyl] pyrrolidine- 2-carboxamide |
| 142 | 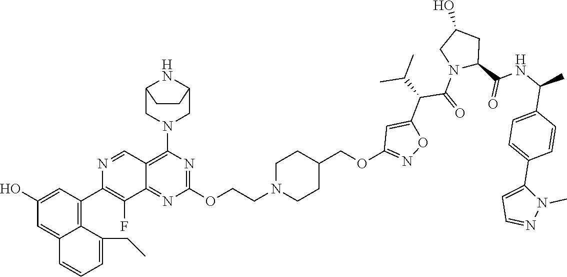 [SMILES: CCc1cccc2cc(O)cc(-c3ncc4c(N5CC6CCC(C5)N6)nc(OCCN5CCC(COc6cc([C@@H](C(=O)N7C[C@H](O)C[C@H]7C(=O)N[C@@H](C)c7ccc(-c8ccnn8C)cc7)C(C)C)on6)CC5)nc4c3F)c12] | (2S,4R*)-1-[(2S)-2- {3-[(1-{2-[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(1- methyl-1H-pyrazol-5- yl)phenyl]ethyl] pyrrolidine- 2-carboxamide |
| 143 | 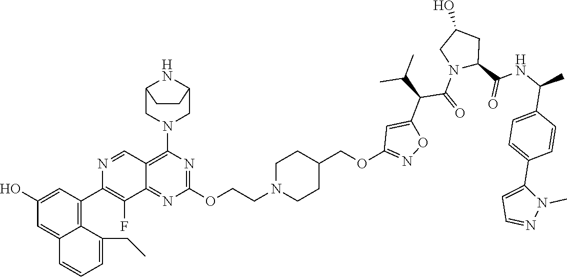 [SMILES: CCc1cccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8ccnn8C)cc7)C(C)C)on6)CC5)nc4c3F)c12] | (2S,4R)-1-[(2R*)-2- {3-[(1-{2-[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]ethyl}piperidin-4- yl)methoxy]-1,2-oxazol-5- yl}-3-methylbutanoyl]-4- hydroxy-N-[(1S)-1-[4-(1- methyl-1H-pyrazol-5- yl)phenyl]ethyl] pyrrolidine- 2-carboxamide |
| 144 | 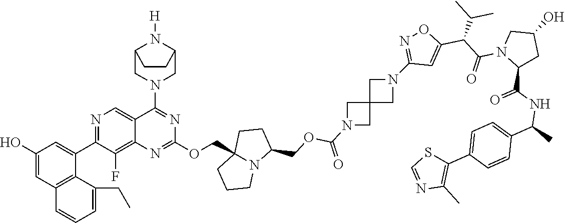 [SMILES: CCc1cccc2cc(O)cc(-c3ncc4c(N5CC6CCC(C5)N6)nc(OC[C@]56CCCN5[C@H](COC(=O)N5CC7(C5)CN(c5cc([C@@H](C(=O)N8C[C@H](O)C[C@H]8C(=O)N[C@@H](C)c8ccc(-c9scnc9C)cc8)C(C)C)on5)C7)CC6)nc4c3F)c12] | [(3S,7aR)-7a-{[(4-{3,8- diazabicyclo[3.2.1] octan-3- yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 6-{5-[(2S)-1- [(2S,4R)-4-hydroxy-2- {[(1S)-1-[4-(4-methyl-1,3- thiazol-5- yl)phenyl]ethyl]carbamoyl} |
| | | pyrrolidin-1-yl]-3-methyl- |
| | | 1-oxobutan-2-yl]-1,2- |
| | | oxazol-3-yl}-2,6- |
| | | diazaspiro[3.3]heptane-2- |
| | | carboxylate |
| 145 | 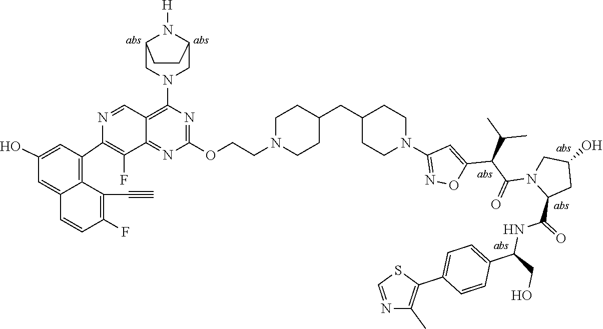 [SMILES: C#Cc1c(F)ccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](CO)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1R)-2- hydroxy-1-[4-(4-methyl- 1,3-thiazol-5- yl)phenyl]ethyl] pyrrolidine- 2-carboxamide |
| 146 | 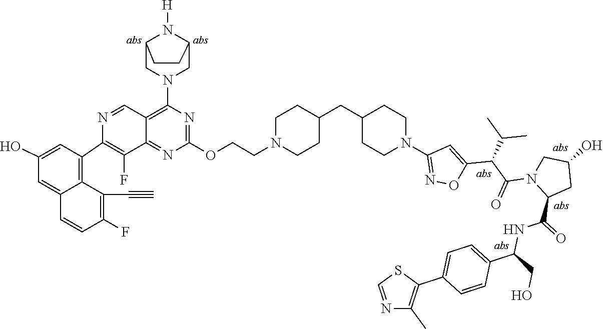 [SMILES: C#Cc1c(F)ccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](CO)c8ccc(-c9scnc9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1R)-2- hydroxy-1-[4-(4-methyl- 1,3-thiazol-5- yl)phenyl]ethyl] pyrrolidine- 2-carboxamide |
| 147 | 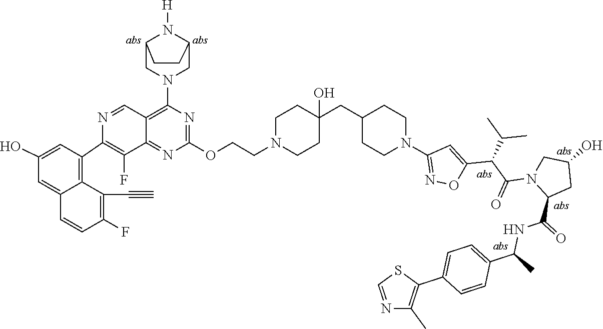 [SMILES: C#Cc1c(F)ccc2cc(O)cc(-c3ncc4c(N5CC6CCC(C5)N6)nc(OCCN5CCC(O)(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- hydroxypiperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 148 | 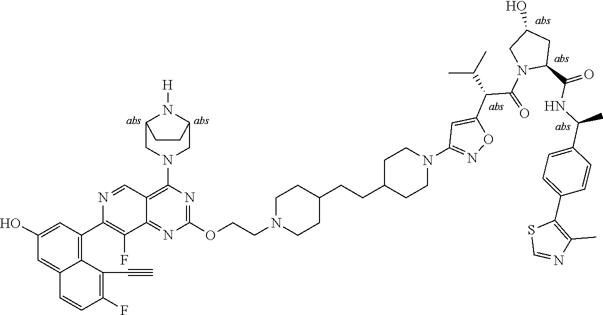 [SMILES: C#Cc1c(F)ccc2cc(O)cc(-c3ncc4c(N5CC6CCC(C5)N6)nc(OCCN5CCC(CCC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2S)-2-(3-{4- [2-(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)ethyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 149 | 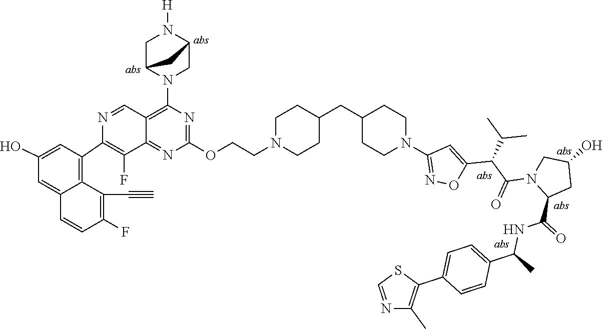 [SMILES: C#Cc1c(F)ccc2cc(O)cc(-c3ncc4c(N5C[C@@H]6C[C@H]5CN6)nc(OCCN5CCC(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2S)-2-{3-[4- ({1-[2-({4-[(1S,4S)-2,5- diazabicyclo[2.2.1]heptan- 2-yl]-7-(8-ethynyl-7- fluoro-3- hydroxynaphthalen-1-yl)- 8-fluoropyrido[4,3- d]pyrimidin-2- yl}oxy)ethyl]piperidin-4- yl}methyl)piperidin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 150 | 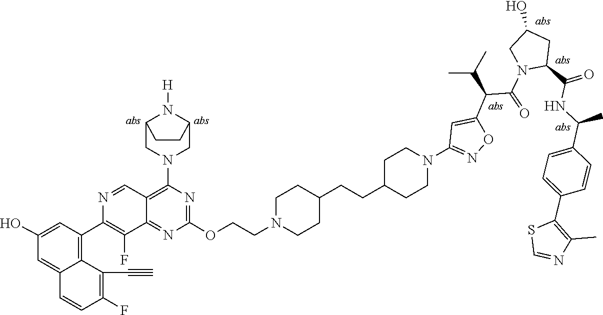 [SMILES: C#Cc1c(F)ccc2cc(O)cc(-c3ncc4c(N5CC6CCC(C5)N6)nc(OCCN5CCC(CCC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [2-(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)ethyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 151 | 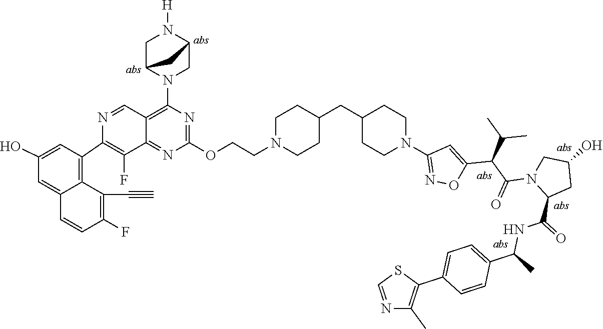 [SMILES: C#Cc1c(F)ccc2cc(O)cc(-c3ncc4c(N5C[C@@H]6C[C@H]5CN6)nc(OCCN5CCC(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-{3-[4- ({1-[2-({4-[(1S,4S)-2,5- diazabicyclo[2.2.1]heptan- 2-yl]-7-(8-ethynyl-7- fluoro-3- hydroxynaphthalen-1-yl)- 8-fluoropyrido[4,3- d]pyrimidin-2- yl}oxy)ethyl]piperidin-4- yl}methyl)piperidin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 152 | 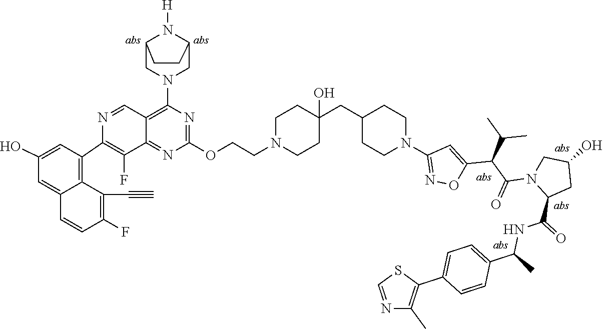 [SMILES: C#Cc1c(F)ccc2cc(O)cc(-c3ncc4c(N5CC6CCC(C5)N6)nc(OCCN5CCC(O)(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- hydroxypiperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 153 | 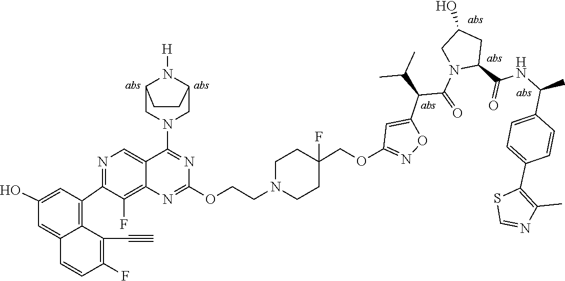 [SMILES: C#Cc1c(F)ccc2cc(O)cc(-c3ncc4c(N5CC6CCC(C5)N6)nc(OCCN5CCC(F)(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- fluoropiperidin-4- yl)methoxy]-1,2-oxazol- 5-yl}-3-methylbutanoyl]- 4-hydroxy-N-[(1S)-1-[4- (4-methyl-1,3-thiazol-5- yl)phenyl]ethyl] pyrrolidine- 2-carboxamide |
| 154 | 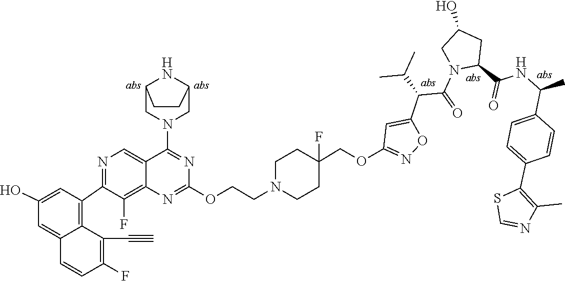 [SMILES: C#Cc1c(F)ccc2cc(O)cc(-c3ncc4c(N5CC6CCC(C5)N6)nc(OCCN5CCC(F)(COc6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2S)-2-{3-[(1- {2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- fluoropiperidin-4- yl)methoxy]-1,2-oxazol- 5-yl}-3-methylbutanoyl]- 4-hydroxy-N-[(1S)-1-[4- (4-methyl-1,3-thiazol-5- yl)phenyl]ethyl] pyrrolidine- 2-carboxamide |
| 155 | 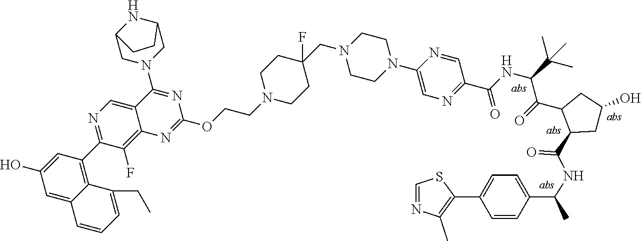 [SMILES: CCc1cccc2cc(O)cc(-c3ncc4c(N5CC6CCC(C5)N6)nc(OCCN5CCC(F)(CN6CCN(c7cnc(C(=O)N[C@H](C(=O)C8C[C@H](O)C[C@H]8C(=O)N[C@@H](C)c8ccc(-c9scnc9C)cc8)C(C)(C)C)cn7)CC6)CC5)nc4c3F)c12] | 5-{4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]piperazin-1-yl}- N-[(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3,3- dimethyl-1-oxobutan-2- |
| | | yl]pyrazine-2- |
| | | carboxamide |
| 156 | 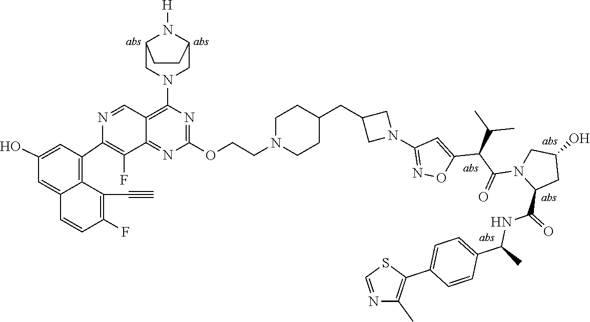 [SMILES: C#Cc1c(F)ccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-(3-{3- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 157 | 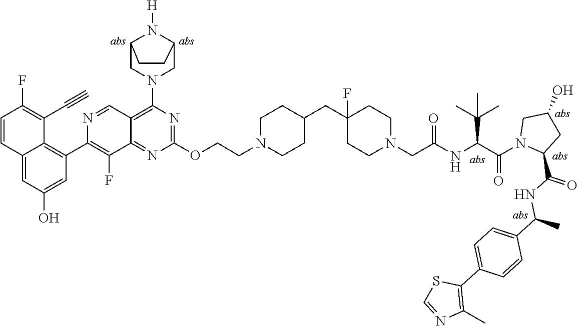 [SMILES: C#Cc1c(F)ccc2cc(O)cc(-c3ncc4c(N5CC6CCC(C5)N6)nc(OCCN5CCC(CC6(F)CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12] | (2S,4R)-1-[(2S)-2-(2-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]-4- fluoropiperidin-1- yl}acetamido)-3,3- dimethylbutanoyl]-4- hydroxy-N-[(1S)-1-[4-(4- methyl-1,3-thiazol-5- yl)phenyl]ethyl] pyrrolidine- 2-carboxamide |
| 158 | 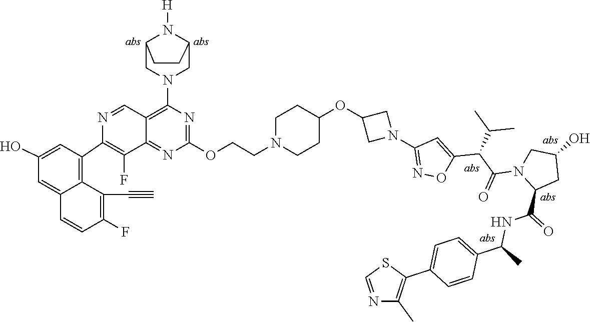 [SMILES: C#Cc1c(F)ccc2cc(O)cc(-c3ncc4c(N5CC6CCC(C5)N6)nc(OCCN5CCC(OC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2S)-2-(3-{3- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)oxy]azetidin-1-yl}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 159 | 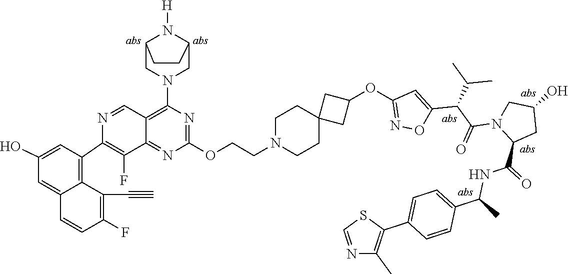 [SMILES: C#Cc1c(F)ccc2cc(O)cc(-c3ncc4c(N5CC6CCC(C5)N6)nc(OCCN5CCC6(CC5)CC(Oc5cc([C@@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2S)-2-{3-[(7- {2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-7- azaspiro[3.5]nonan-2- yl)oxy]-1,2-oxazol-5-yl}- 3-methylbutanoyl]-4- hydroxy-N-[(1S)-1-[4-(4- methyl-1,3-thiazol-5- yl)phenyl]ethyl] pyrrolidine- 2-carboxamide |
| 160 | 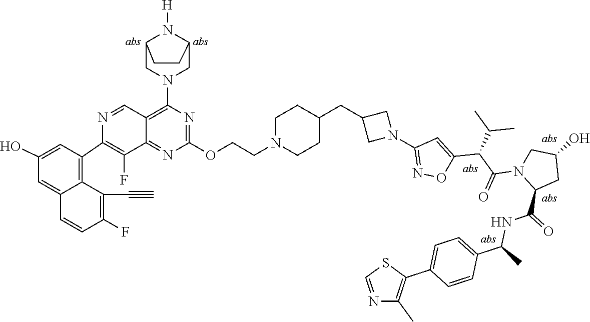 [SMILES: C#Cc1c(F)ccc2cc(O)cc(-c3ncc4c(N5CC6CCC(C5)N6)nc(OCCN5CCC(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2S)-2-(3-{3- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 161 | 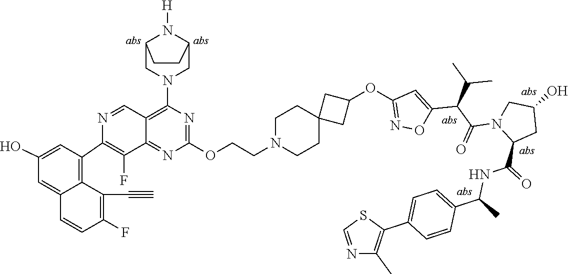 [SMILES: C#Cc1c(F)ccc2cc(O)cc(-c3ncc4c(N5CC6CCC(C5)N6)nc(OCCN5CCC6(CC5)CC(Oc5cc([C@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2R)-2-{3-[(7- {2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-7- azaspiro[3.5]nonan-2- yl)oxy]-1,2-oxazol-5-yl}- 3-methylbutanoyl]-4- hydroxy-N-[(1S)-1-[4-(4- methyl-1,3-thiazol-5- yl)phenyl]ethyl] pyrrolidine- 2-carboxamide |
| 162 | 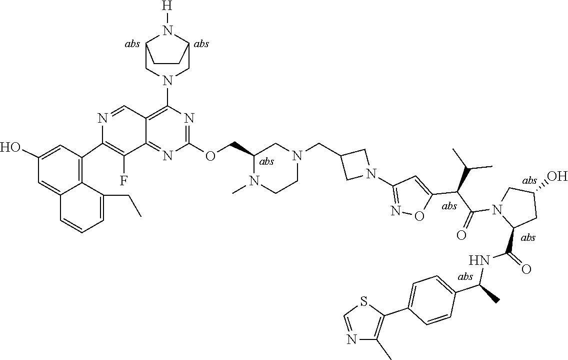 [SMILES: CCc1cccc2cc(O)cc(-c3ncc4c(N5CC6CCC(C5)N6)nc(OC[C@H]5CN(CC6CN(c7cc([C@H](C(=O)N8C[C@H](O)C[C@H]8C(=O)N[C@@H](C)c8ccc(-c9scnc9C)cc8)C(C)C)on7)C6)CCN5C)nc4c3F)c12] | (2S,4R)-1-[(2R)-2-[3-(3- {[(3R)-3-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-4- methylpiperazin-1- yl]methyl}azetidin-1-yl)- 1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 163 | 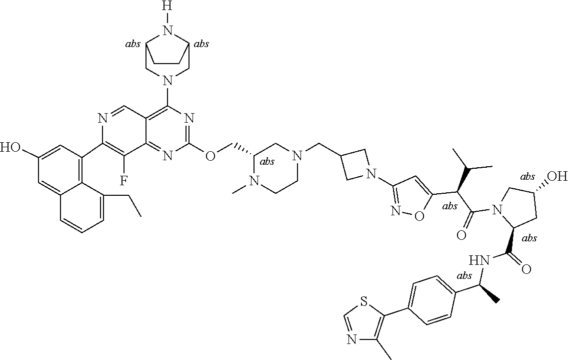 [SMILES: CCc1cccc2cc(O)cc(-c3ncc4c(N5CC6CCC(C5)N6)nc(OC[C@@H]5CN(CC6CN(c7cc([C@H](C(=O)N8C[C@H](O)C[C@H]8C(=O)N[C@@H](C)c8ccc(-c9scnc9C)cc8)C(C)C)on7)C6)CCN5C)nc4c3F)c12] | (2S,4R)-1-[(2R)-2-[3-(3- {[(3S)-3-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-4- methylpiperazin-1- yl]methyl}azetidin-1-yl)- 1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 164 | 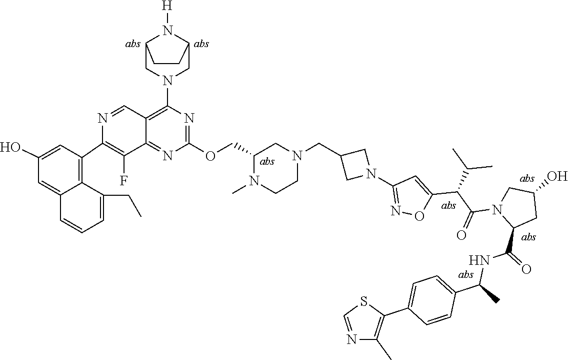 [SMILES: CCc1cccc2cc(O)cc(-c3ncc4c(N5CC6CCC(C5)N6)nc(OC[C@@H]5CN(CC6CN(c7cc([C@@H](C(=O)N8C[C@H](O)C[C@H]8C(=O)N[C@@H](C)c8ccc(-c9scnc9C)cc8)C(C)C)on7)C6)CCN5C)nc4c3F)c12] | (2S,4R)-1-[(2S)-2-[3-(3- {[(3S)-3-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-4- methylpiperazin-1- yl]methyl}azetidin-1-yl)- 1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 165 | 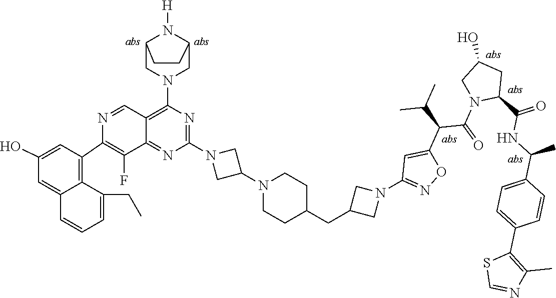 [SMILES: CCc1cccc2cc(O)cc(-c3ncc4c(N5CC6CCC(C5)N6)nc(N5CC(N6CCC(CC7CN(c8cc([C@H](C(=O)N9C[C@H](O)C[C@H]9C(=O)N[C@@H](C)c9ccc(-c%10scnc%10C)cc9)C(C)C)on8)C7)CC6)C5)nc4c3F)c12] | (2S,4R)-1-[(2R)-2-{3-[3- ({1-[1-(4-{3,8- diazabicyclo[3.2.1]octan- 3-yl}-7-(8-ethyl-3- hydroxynaphthalen-1-yl)- 8-fluoropyrido[4,3- d]pyrimidin-2-yl)azetidin- 3-yl]piperidin-4- yl}methyl)azetidin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 166 | 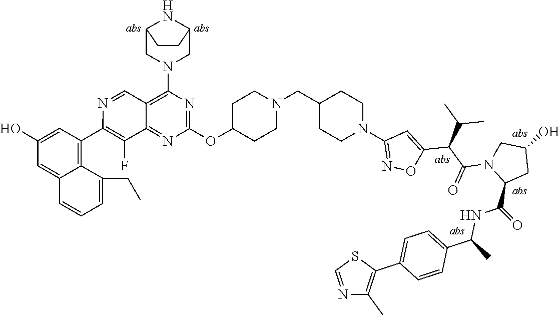 [SMILES: CCc1cccc2cc(O)cc(-c3ncc4c(N5CC6CCC(C5)N6)nc(OC5CCN(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-{3-[4- ({4-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]piperidin-1- yl}methyl)piperidin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 167 | 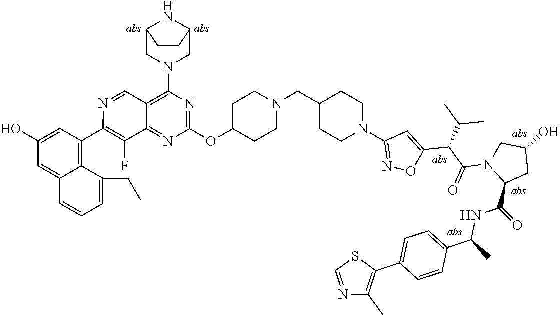 [SMILES: CCc1cccc2cc(O)cc(-c3ncc4c(N5CC6CCC(C5)N6)nc(OC5CCN(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2S)-2-{3-[4- ({4-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]piperidin-1- yl}methyl)piperidin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 168 | 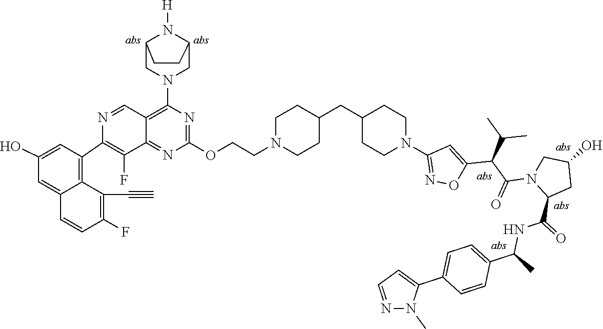 [SMILES: C#Cc1c(F)ccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 169 | 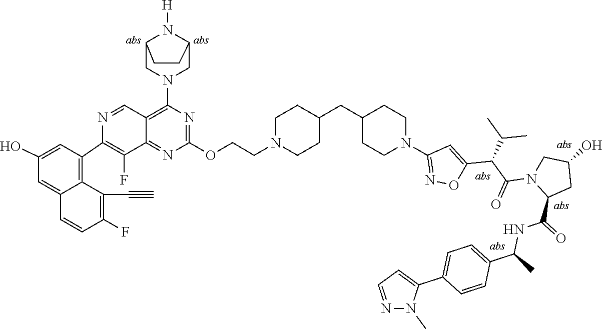 [SMILES: C#Cc1c(F)ccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 170 | 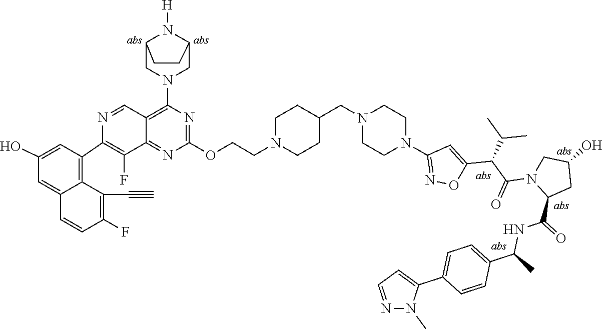 [SMILES: C#Cc1c(F)ccc2cc(O)cc(-c3ncc4c(N5CC6CCC(C5)N6)nc(OCCN5CCC(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperazin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 171 | 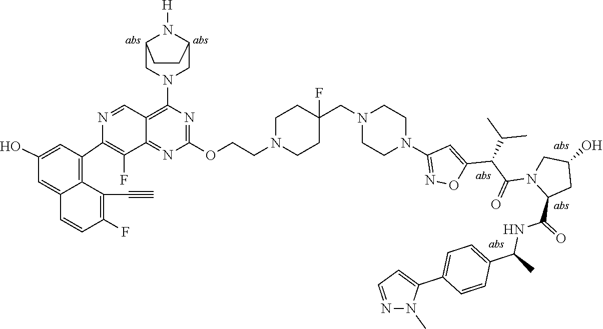 [SMILES: C#Cc1c(F)ccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- fluoropiperidin-4- yl)methyl]piperazin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 172 | 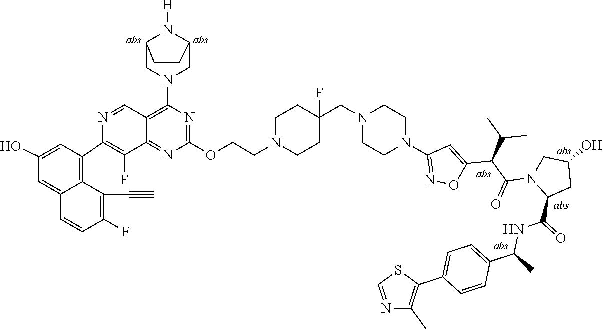 [SMILES: C#Cc1c(F)ccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- fluoropiperidin-4- yl)methyl]piperazin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 173 | 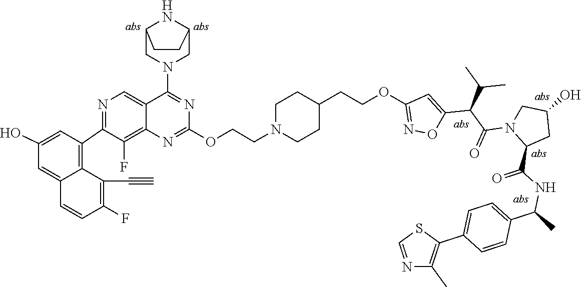 [SMILES: C#Cc1c(F)ccc2cc(O)cc(-c3ncc4c(N5CC6CCC(C5)N6)nc(OCCN5CCC(CCOc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[2- (1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)ethoxy]-1,2-oxazol-5- yl}-3-methylbutanoyl]-4- hydroxy-N-[(1S)-1-[4-(4- methyl-1,3-thiazol-5- yl)phenyl]ethyl] pyrrolidine- 2-carboxamide |
| 174 | 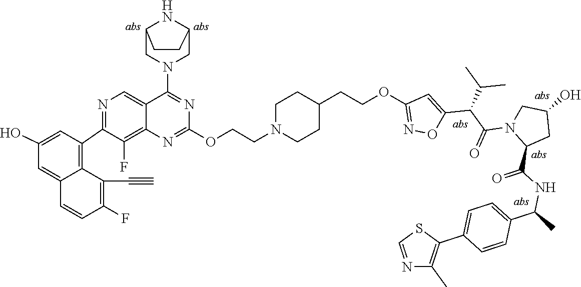 [SMILES: C#Cc1c(F)ccc2cc(O)cc(-c3ncc4c(N5CC6CCC(C5)N6)nc(OCCN5CCC(CCOc6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2S)-2-{3-[2- (1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)ethoxy]-1,2-oxazol-5- yl}-3-methylbutanoyl]-4- hydroxy-N-[(1S)-1-[4-(4- methyl-1,3-thiazol-5- yl)phenyl]ethyl] pyrrolidine- 2-carboxamide |
| 175 | 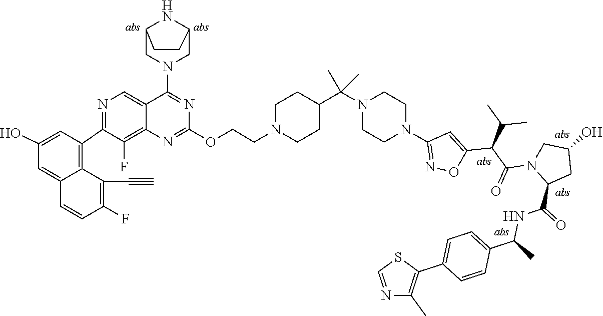 [SMILES: C#Cc1c(F)ccc2cc(O)cc(-c3ncc4c(N5CC6CCC(C5)N6)nc(OCCN5CCC(C(C)(C)N6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [2-(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)propan-2-yl]piperazin- 1-yl}-1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 176 | 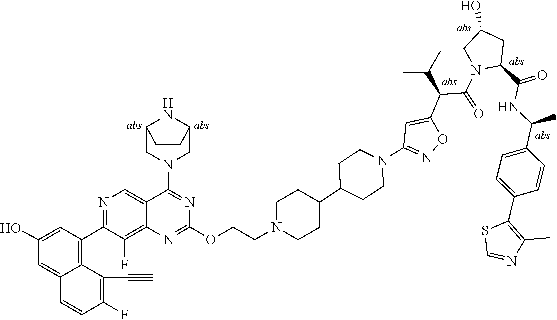 [SMILES: C#Cc1c(F)ccc2cc(O)cc(-c3ncc4c(N5CC6CCC(C5)N6)nc(OCCN5CCC(C6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-[3-(1′- {2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-[4,4′- bipiperidin]-1-yl)-1,2- oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 177 | 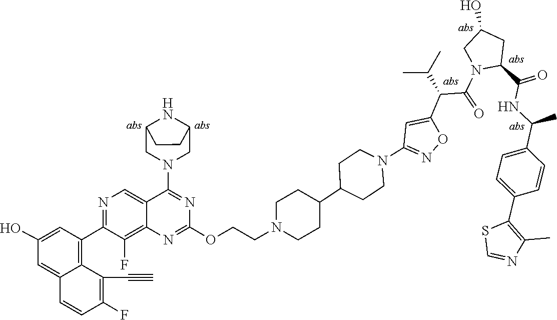 [SMILES: C#Cc1c(F)ccc2cc(O)cc(-c3ncc4c(N5CC6CCC(C5)N6)nc(OCCN5CCC(C6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2S)-2-[3-(1′- {2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-[4,4′- bipiperidin]-1-yl)-1,2- oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 178 | 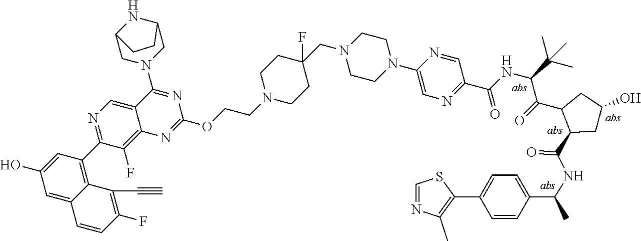 [SMILES: C#Cc1c(F)ccc2cc(O)cc(-c3ncc4c(N5CC6CCC(C5)N6)nc(OCCN5CCC(F)(CN6CCN(c7cnc(C(=O)N[C@H](C(=O)C8C[C@H](O)C[C@H]8C(=O)N[C@@H](C)c8ccc(-c9scnc9C)cc8)C(C)(C)C)cn7)CC6)CC5)nc4c3F)c12] | 5-{4-[(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- fluoropiperidin-4- yl)methyl]piperazin-1-yl}- N-[(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3,3- |
| | | dimethyl-1-oxobutan-2- |
| | | yl]pyrazine-2- |
| | | carboxamide |
| 179 | 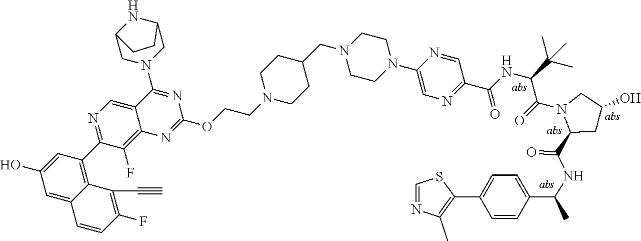 [SMILES: C#Cc1c(F)ccc2cc(O)cc(-c3ncc4c(N5CC6CCC(C5)N6)nc(OCCN5CCC(CN6CCN(c7cnc(C(=O)N[C@H](C(=O)N8C[C@H](O)C[C@H]8C(=O)N[C@@H](C)c8ccc(-c9scnc9C)cc8)C(C)(C)C)cn7)CC6)CC5)nc4c3F)c12] | 5-{4-[(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperazin-1-yl}- N-[(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3,3- dimethyl-1-oxobutan-2- yl]pyrazine-2- |
| | | carboxamide |
| 180 | 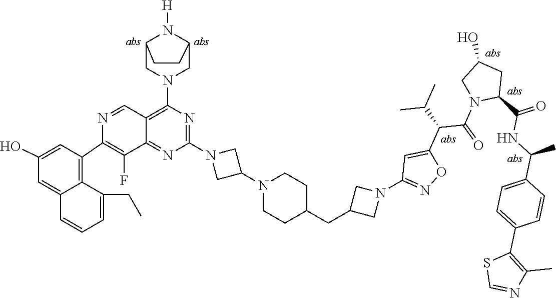 [SMILES: CCc1cccc2cc(O)cc(-c3ncc4c(N5CC6CCC(C5)N6)nc(N5CC(N6CCC(CC7CN(c8cc([C@@H](C(=O)N9C[C@H](O)C[C@H]9C(=O)N[C@@H](C)c9ccc(-c%10scnc%10C)cc9)C(C)C)on8)C7)CC6)C5)nc4c3F)c12] | (2S,4R)-1-[(2S)-2-{3-[3- ({1-[1-(4-{3,8- diazabicyclo[3.2.1]octan- 3-yl}-7-(8-ethyl-3- hydroxynaphthalen-1-yl)- 8-fluoropyrido[4,3- d]pyrimidin-2-yl)azetidin- 3-yl]piperidin-4- yl}methyl)azetidin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 181 | 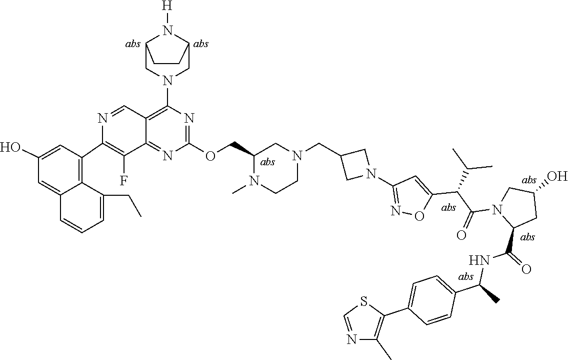 [SMILES: CCc1cccc2cc(O)cc(-c3ncc4c(N5CC6CCC(C5)N6)nc(OC[C@H]5CN(CC6CN(c7cc([C@@H](C(=O)N8C[C@H](O)C[C@H]8C(=O)N[C@@H](C)c8ccc(-c9scnc9C)cc8)C(C)C)on7)C6)CCN5C)nc4c3F)c12] | (2S,4R)-1-[(2S)-2-[3-(3- {[(3R)-3-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-4- methylpiperazin-1- yl]methyl}azetidin-1-yl)- 1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 182 | 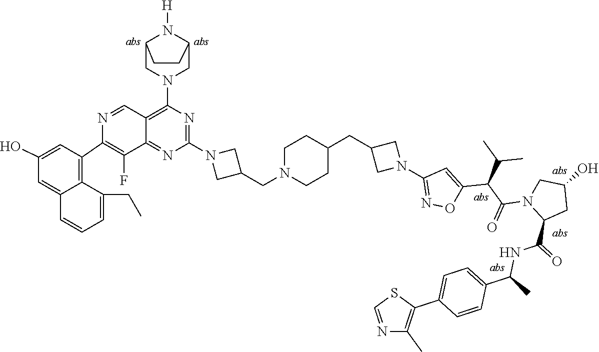 [SMILES: CCc1cccc2cc(O)cc(-c3ncc4c(N5CC6CCC(C5)N6)nc(N5CC(CN6CCC(CC7CN(c8cc([C@H](C(=O)N9C[C@H](O)C[C@H]9C(=O)N[C@@H](C)c9ccc(-c%10scnc%10C)cc9)C(C)C)on8)C7)CC6)C5)nc4c3F)c12] | (2S,4R)-1-[(2R)-2-(3-{3- [(1-{[1-(4-{3,8- diazabicyclo[3.2.1]octan- 3-yl}-7-(8-ethyl-3- hydroxynaphthalen-1-yl)- 8-fluoropyrido[4,3- d]pyrimidin-2-yl)azetidin- 3-yl]methyl}piperidin-4- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 183 | 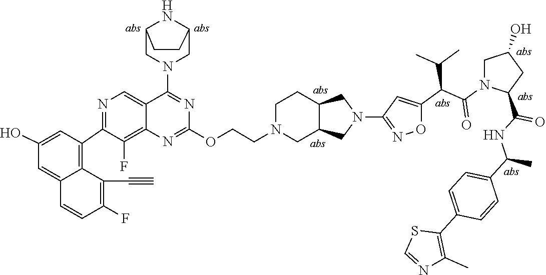 [SMILES: C#Cc1c(F)ccc2cc(O)cc(-c3ncc4c(N5CC6CCC(C5)N6)nc(OCCN5CC[C@H]6CN(c7cc([C@H](C(=O)N8C[C@H](O)C[C@H]8C(=O)N[C@@H](C)c8ccc(-c9scnc9C)cc8)C(C)C)on7)C[C@H]6C5)nc4c3F)c12] | (2S,4R)-1-[(2R)-2-{3- [(3aR,7aR)-5-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-octahydro- 1H-pyrrolo[3,4-c]pyridin- 2-yl]-1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 184 | 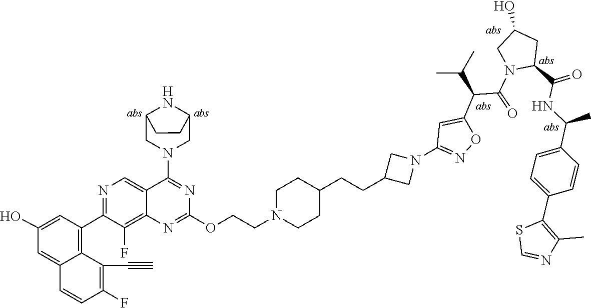 [SMILES: C#Cc1c(F)ccc2cc(O)cc(-c3ncc4c(N5CC6CCC(C5)N6)nc(OCCN5CCC(CCC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-(3-{3- [2-(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)ethyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 185 | 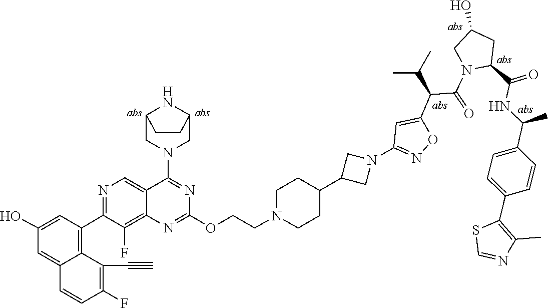 [SMILES: C#Cc1c(F)ccc2cc(O)cc(-c3ncc4c(N5CC6CCC(C5)N6)nc(OCCN5CCC(C6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-{3-[3- (1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)azetidin-1-yl]-1,2- oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 186 | 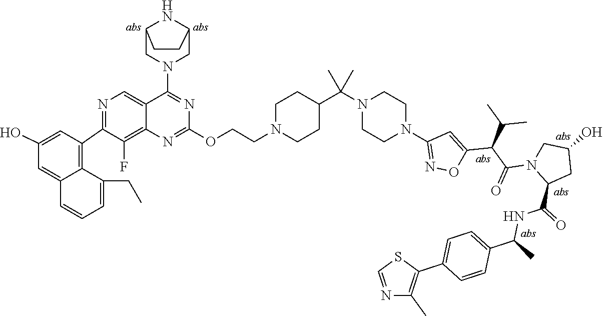 [SMILES: CCc1cccc2cc(O)cc(-c3ncc4c(N5CC6CCC(C5)N6)nc(OCCN5CCC(C(C)(C)N6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [2-(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)propan-2-yl]piperazin- 1-yl}-1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 187 | 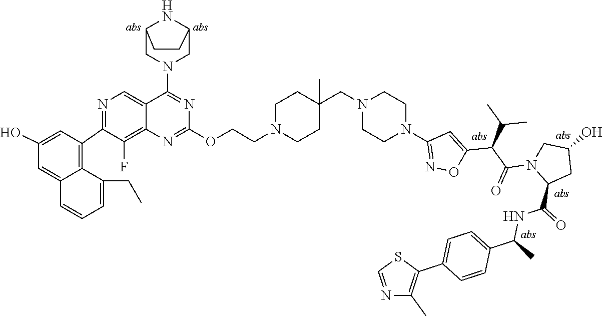 [SMILES: CCc1cccc2cc(O)cc(-c3ncc4c(N5CC6CCC(C5)N6)nc(OCCN5CCC(C)(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- methylpiperidin-4- yl)methyl]piperazin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 188 | 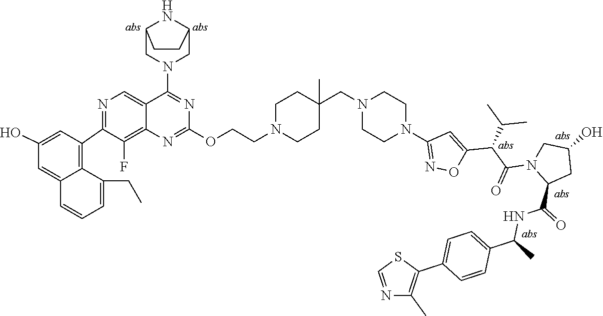 [SMILES: CCc1cccc2cc(O)cc(-c3ncc4c(N5CC6CCC(C5)N6)nc(OCCN5CCC(C)(CN6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- methylpiperidin-4- yl)methyl]piperazin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 189 | 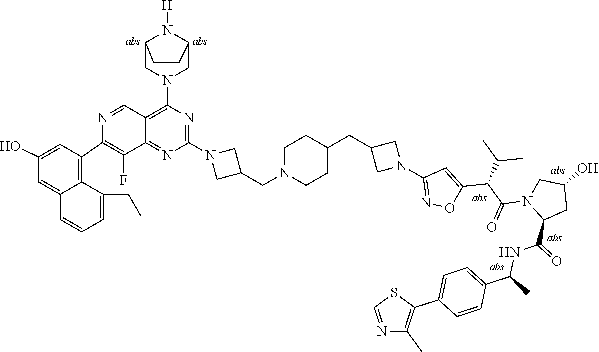 [SMILES: CCc1cccc2cc(O)cc(-c3ncc4c(N5CC6CCC(C5)N6)nc(N5CC(CN6CCC(CC7CN(c8cc([C@@H](C(=O)N9C[C@H](O)C[C@H]9C(=O)N[C@@H](C)c9ccc(-c%10scnc%10C)cc9)C(C)C)on8)C7)CC6)C5)nc4c3F)c12] | (2S,4R)-1-[(2S)-2-(3-{3- [(1-{[1-(4-{3,8- diazabicyclo[3.2.1]octan- 3-yl}-7-(8-ethyl-3- hydroxynaphthalen-1-yl)- 8-fluoropyrido[4,3- d]pyrimidin-2-yl)azetidin- 3-yl]methyl}piperidin-4- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 190 | 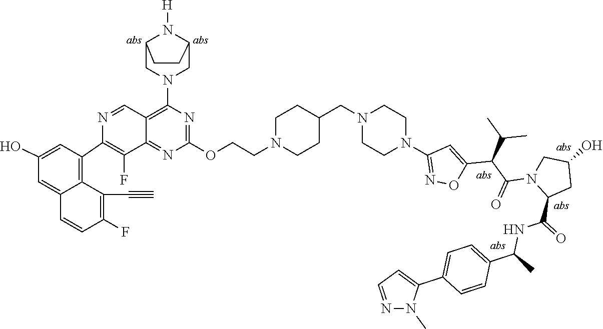 [SMILES: C#Cc1c(F)ccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperazin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 191 | 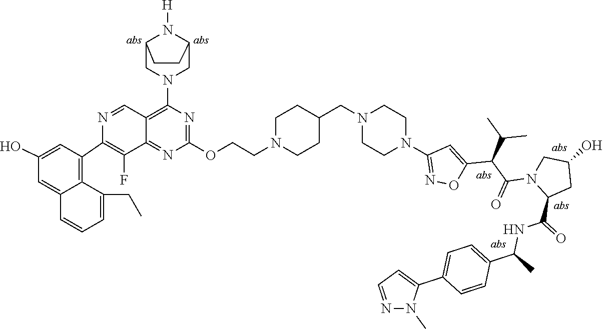 [SMILES: CCc1cccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperazin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 192 | 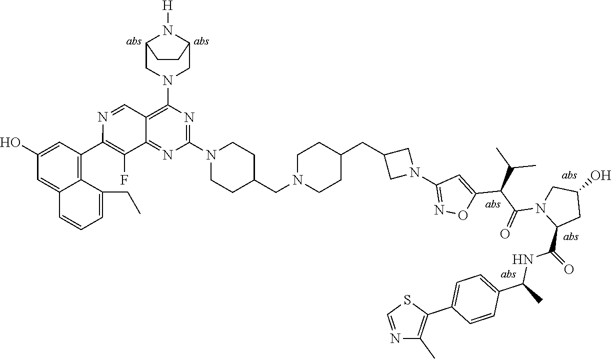 [SMILES: CCc1cccc2cc(O)cc(-c3ncc4c(N5CC6CCC(C5)N6)nc(N5CCC(CN6CCC(CC7CN(c8cc([C@H](C(=O)N9C[C@H](O)C[C@H]9C(=O)N[C@@H](C)c9ccc(-c%10scnc%10C)cc9)C(C)C)on8)C7)CC6)CC5)nc4c3F)c12] | (2S,4R)-1-[(2R)-2-(3-{3- [(1-{[1-(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)piperidin-4- yl]methyl}piperidin-4- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 193 | 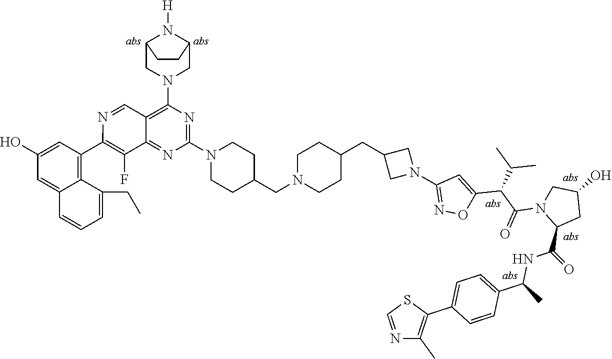 [SMILES: CCc1cccc2cc(O)cc(-c3ncc4c(N5CC6CCC(C5)N6)nc(N5CCC(CN6CCC(CC7CN(c8cc([C@@H](C(=O)N9C[C@H](O)C[C@H]9C(=O)N[C@@H](C)c9ccc(-c%10scnc%10C)cc9)C(C)C)on8)C7)CC6)CC5)nc4c3F)c12] | (2S,4R)-1-[(2S)-2-(3-{3- [(1-{[1-(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)piperidin-4- yl]methyl}piperidin-4- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 194 | 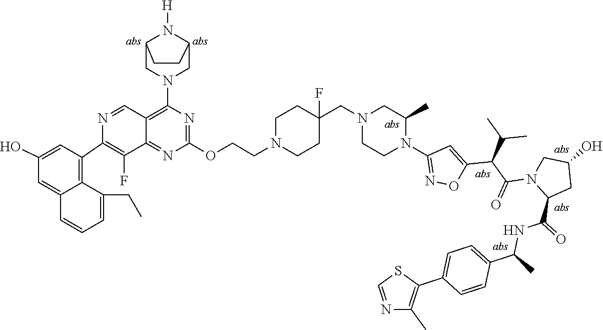 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)[C@H](C)C6)CC5)nc4c3F)c12] | (2S,4R)-1-[(2R)-2-{3- [(2R)-4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]-2- methylpiperazin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 195 | 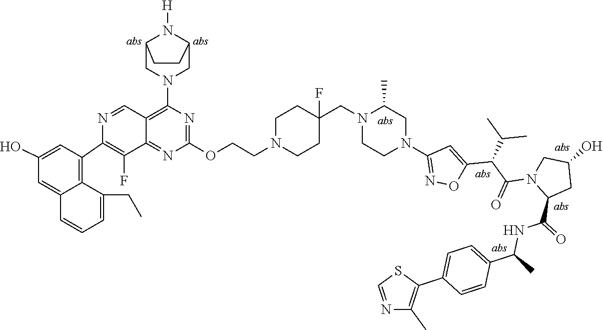 [SMILES: CCc1cccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)C[C@H]6C)CC5)nc4c3F)c12] | (2S,4R)-1-[(2S)-2-{3- [(3R)-4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]-3- methylpiperazin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 196 | 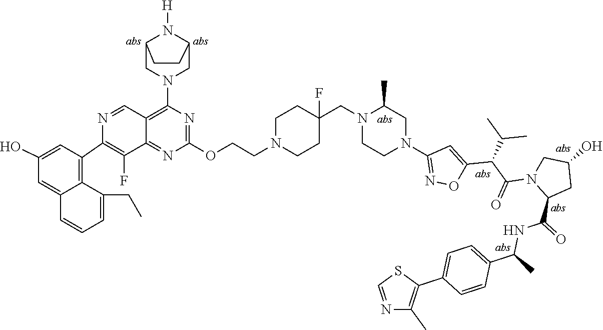 [SMILES: CCc1cccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)C[C@@H]6C)CC5)nc4c3F)c12] | (2S,4R)-1-[(2S)-2-{3- [(3S)-4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]-3- methylpiperazin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 197 | 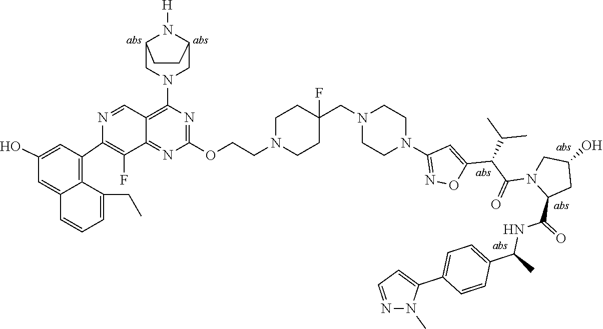 [SMILES: CCc1cccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]piperazin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 198 | 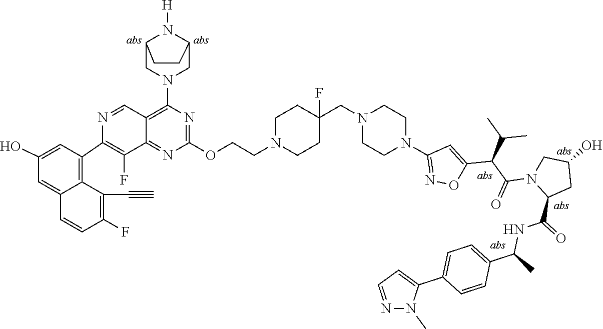 [SMILES: C#Cc1c(F)ccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- fluoropiperidin-4- yl)methyl]piperazin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 199 | 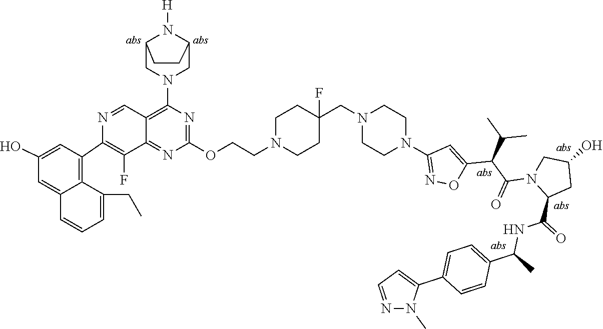 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]piperazin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 200 | 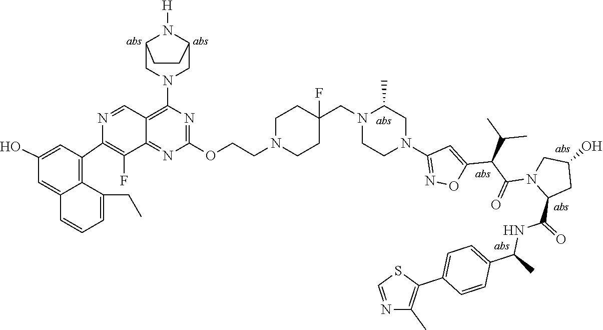 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)C[C@H]6C)CC5)nc4c3F)c12] | (2S,4R)-1-[(2R)-2-{3- [(3R)-4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]-3- methylpiperazin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 201 | 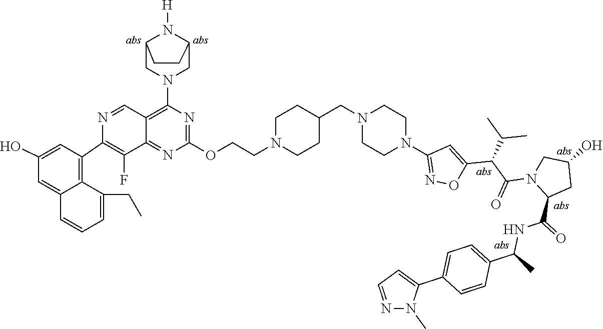 [SMILES: CCc1cccc2cc(O)cc(-c3ncc4c(N5CC6CCC(C5)N6)nc(OCCN5CCC(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperazin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 202 | 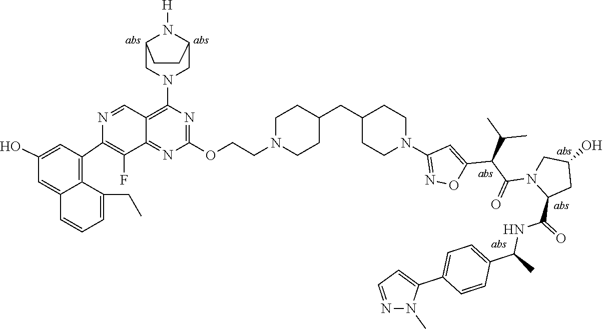 [SMILES: CCc1cccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 203 | 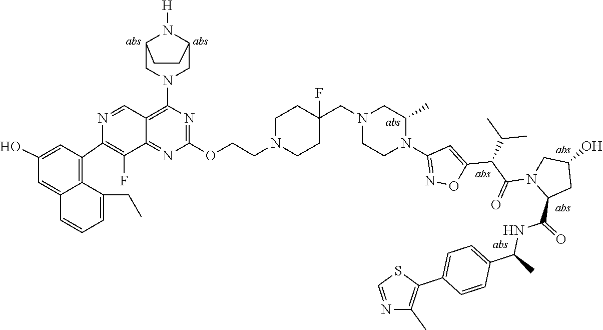 [SMILES: CCc1cccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)[C@@H](C)C6)CC5)nc4c3F)c12] | (2S,4R)-1-[(2S)-2-{3- [(2S)-4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]-2- methylpiperazin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 204 | 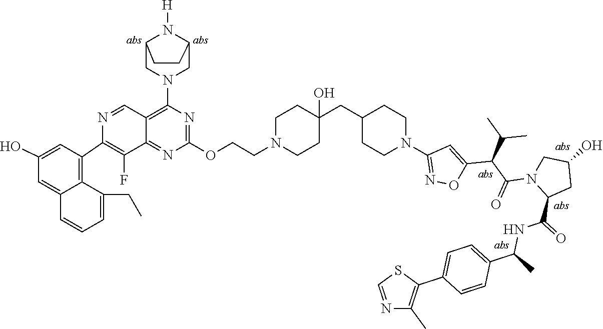 [SMILES: CCc1cccc2cc(O)cc(-c3ncc4c(N5CC6CCC(C5)N6)nc(OCCN5CCC(O)(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- hydroxypiperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 205 | 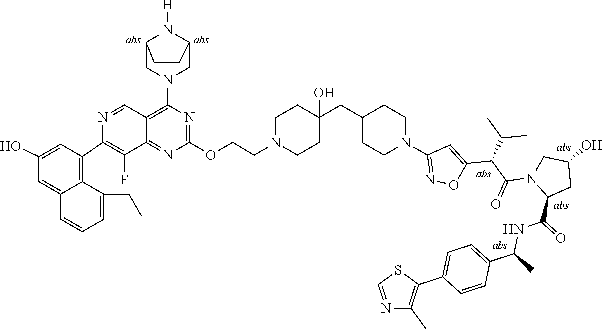 [SMILES: CCc1cccc2cc(O)cc(-c3ncc4c(N5CC6CCC(C5)N6)nc(OCCN5CCC(O)(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- hydroxypiperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 206 | 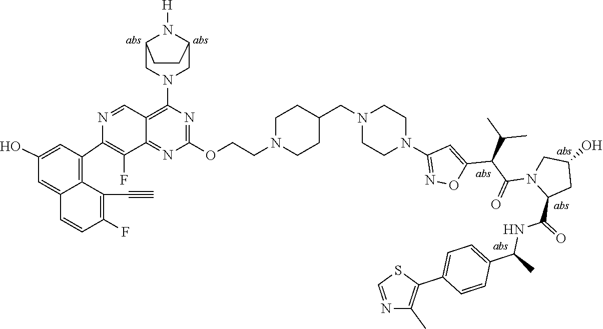 [SMILES: C#Cc1c(F)ccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperazin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 207 | 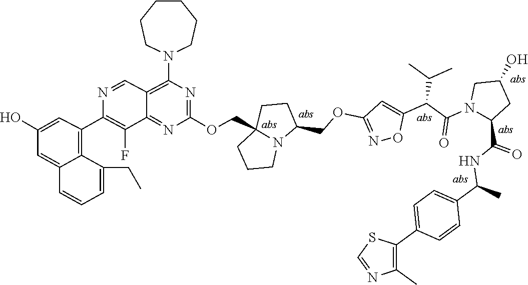 [SMILES: CCc1cccc2cc(O)cc(-c3ncc4c(N5CCCCCC5)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 208 | 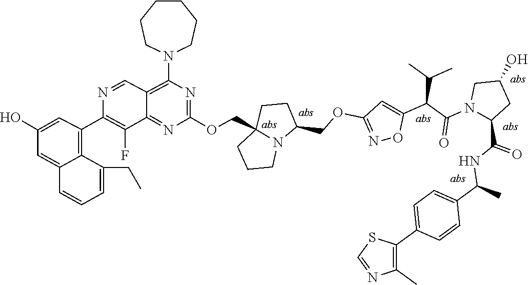 [SMILES: CCc1cccc2cc(O)cc(-c3ncc4c(N5CCCCCC5)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 209 | 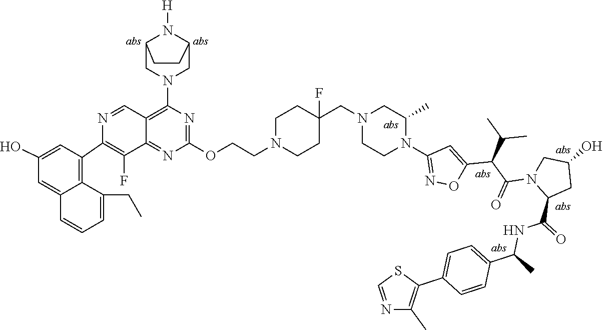 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)[C@@H](C)C6)CC5)nc4c3F)c12] | (2S,4R)-1-[(2R)-2-{3- [(2S)-4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]-2- methylpiperazin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 210 | 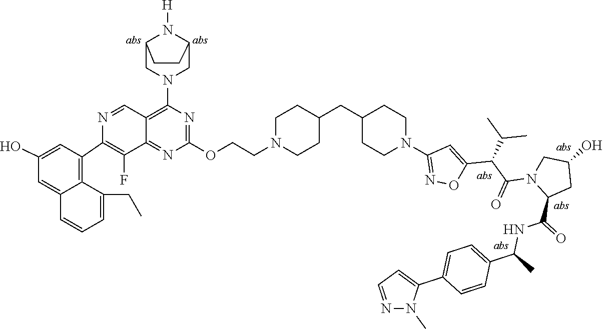 [SMILES: CCc1cccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 211 | 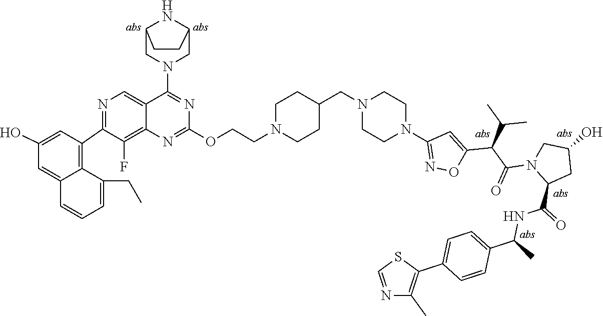 [SMILES: CCc1cccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperazin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 212 | 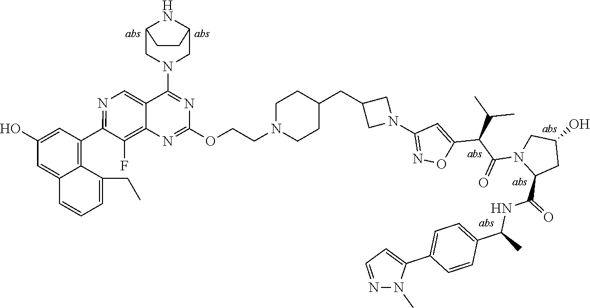 [SMILES: CCc1cccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9ccnn9C)cc8)C(C)C)on7)C6)CC5)nc4c3F)c12] | (2S,4R)-1-[(2R)-2-(3-{3- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 213 | 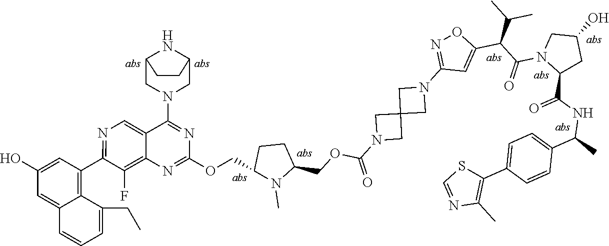 [SMILES: CCc1cccc2cc(O)cc(-c3ncc4c(N5CC6CCC(C5)N6)nc(OC[C@@H]5CC[C@@H](COC(=O)N6CC7(C6)CN(c6cc([C@H](C(=O)N8C[C@H](O)C[C@H]8C(=O)N[C@@H](C)c8ccc(-c9scnc9C)cc8)C(C)C)on6)C7)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 6-{5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3-yl}-2,6- diazaspiro[3.3]heptane- 2-carboxylate |
| 214 | 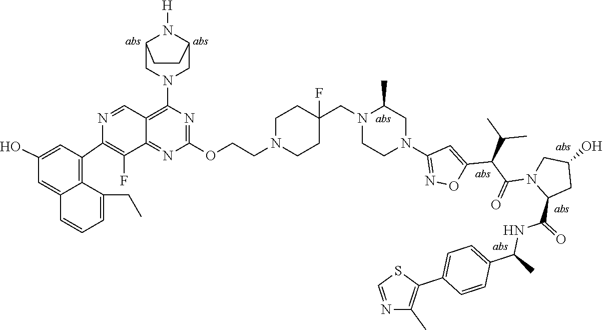 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)C[C@@H]6C)CC5)nc4c3F)c12] | (2S,4R)-1-[(2R)-2-{3- [(3S)-4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]-3- methylpiperazin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 215 | 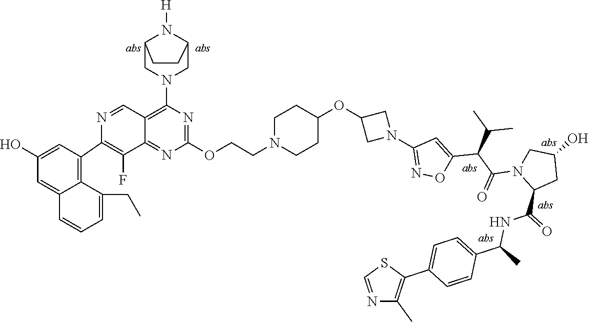 [SMILES: CCc1cccc2cc(O)cc(-c3ncc4c(N5CC6CCC(C5)N6)nc(OCCN5CCC(OC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-(3-{3- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)oxy]azetidin-1-yl}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 216 | 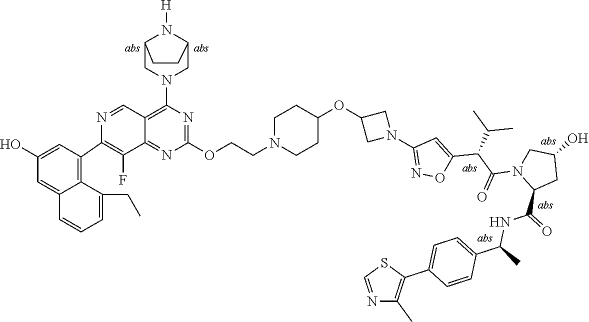 [SMILES: CCc1cccc2cc(O)cc(-c3ncc4c(N5CC6CCC(C5)N6)nc(OCCN5CCC(OC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2S)-2-(3-{3- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)oxy]azetidin-1-yl}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 217 | 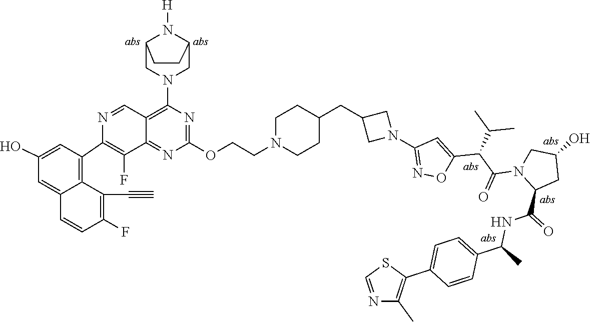 [SMILES: C#Cc1c(F)ccc2cc(O)cc(-c3ncc4c(N5CC6CCC(C5)N6)nc(OCCN5CCC(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2S)-2-(3-{3- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 218 | 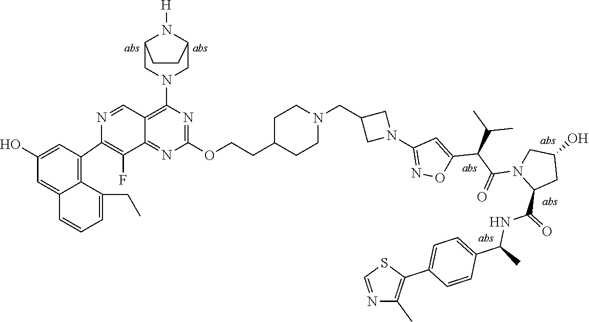 [SMILES: CCc1cccc2cc(O)cc(-c3ncc4c(N5CC6CCC(C5)N6)nc(OCCC5CCN(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-(3-{3- [(4-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-1- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 219 | 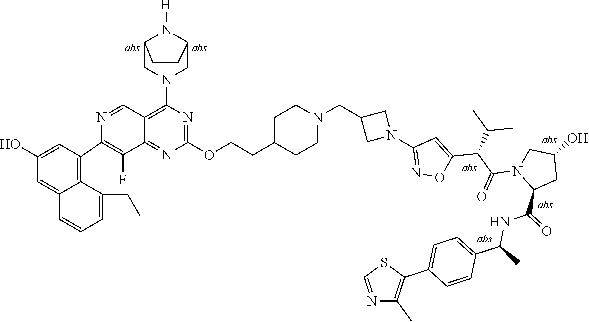 [SMILES: CCc1cccc2cc(O)cc(-c3ncc4c(N5CC6CCC(C5)N6)nc(OCCC5CCN(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2S)-2-(3-{3- [(4-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-1- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 220 | 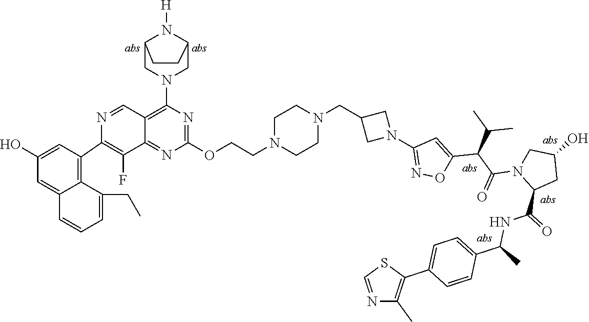 [SMILES: CCc1cccc2cc(O)cc(-c3ncc4c(N5CC6CCC(C5)N6)nc(OCCN5CCN(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-(3-{3- [(4-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperazin-1- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 221 | 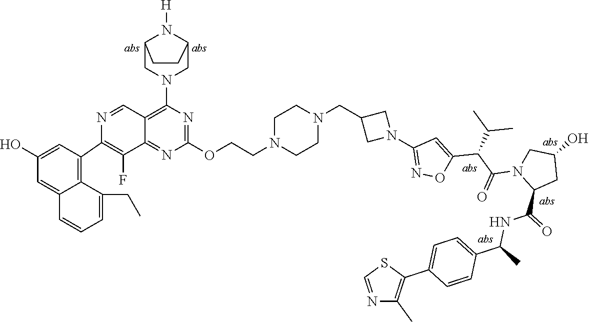 [SMILES: CCc1cccc2cc(O)cc(-c3ncc4c(N5CC6CCC(C5)N6)nc(OCCN5CCN(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2S)-2-(3-{3- [(4-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperazin-1- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 222 | 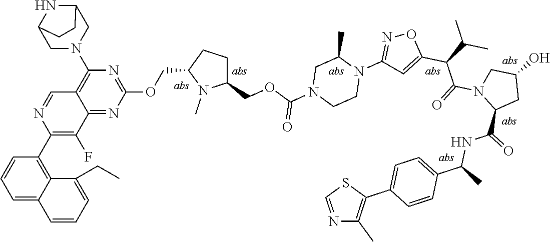 [SMILES: CCc1cccc2cccc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)[C@H](C)C6)N5C)nc4c3F)c12] | [(2S,5S)-5-{[(4-{3,8- diazabicyclo[3.2.1]octan- 3-yl}-7-(8- ethylnaphthalen-1-yl)-8- fluoropyrido[4,3- d]pyrimidin-2- yl)oxy]methyl}-1- methylpyrrolidin-2- yl]methyl (3R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- 1,2-oxazol-3-yl}-3- |
| | | methylpiperazine-1- |
| | | carboxylate |
| 223 | 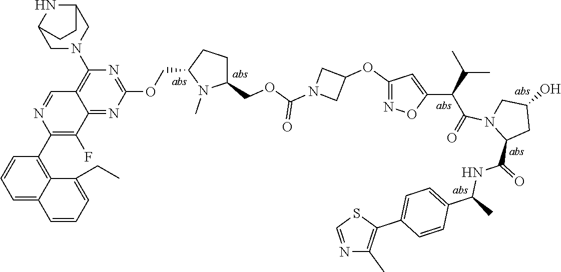 [SMILES: CCc1cccc2cccc(-c3ncc4c(N5CC6CCC(C5)N6)nc(OC[C@@H]5CC[C@@H](COC(=O)N6CC(Oc7cc([C@H](C(=O)N8C[C@H](O)C[C@H]8C(=O)N[C@@H](C)c8ccc(-c9scnc9C)cc8)C(C)C)on7)C6)N5C)nc4c3F)c12] | [(2S,5S)-5-{[(4-{3,8- diazabicyclo[3.2.1]octan- 3-yl}-7-(8- ethylnaphthalen-1-yl)-8- fluoropyrido[4,3- d]pyrimidin-2- yl)oxy]methyl}-1- methylpyrrolidin-2- yl]methyl 3-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- yl}oxy)azetidine-1- |
| | | carboxylate |
| 224 | 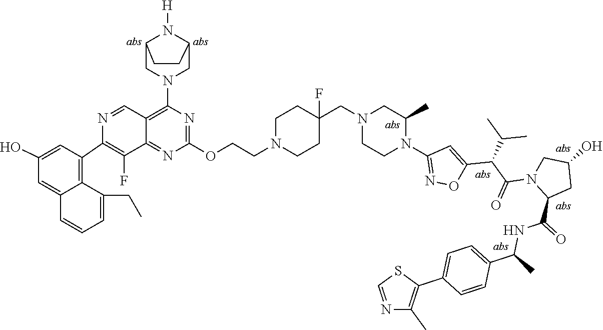 [SMILES: CCc1cccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)[C@H](C)C6)CC5)nc4c3F)c12] | (2S,4R)-1-[(2S)-2-{3- [(2R)-4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]-2- methylpiperazin-1-yl]- 1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 225 | 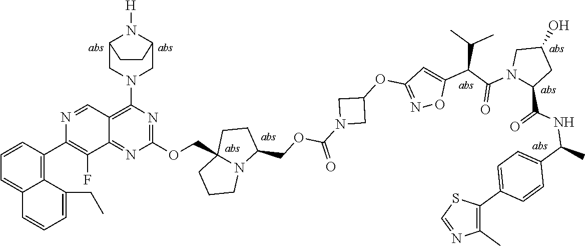 [SMILES: CCc1cccc2cccc(-c3ncc4c(N5CC6CCC(C5)N6)nc(OC[C@]56CCCN5[C@H](COC(=O)N5CC(Oc7cc([C@H](C(=O)N8C[C@H](O)C[C@H]8C(=O)N[C@@H](C)c8ccc(-c9scnc9C)cc8)C(C)C)on7)C5)CC6)nc4c3F)c12] | [(3S,7aR)-7a-{[(4-{3,8- diazabicyclo[3.2.1]octan- 3-yl}-7-(8- ethylnaphthalen-1-yl)-8- fluoropyrido[4,3- d]pyrimidin-2- yl)oxy]methyl}- hexahydro-1H-pyrrolizin- 3-yl]methyl 3-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- |
| | | yl}oxy)azetidine-1- |
| | | carboxylate |
| 226 | 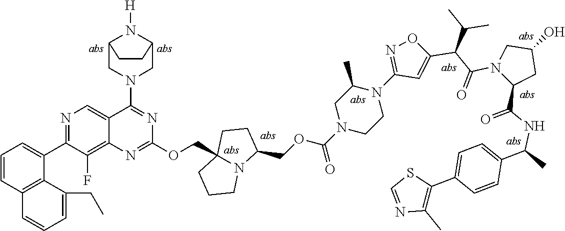 [SMILES: CCc1cccc2cccc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)[C@H](C)C5)CC6)nc4c3F)c12] | [(3S,7aR)-7a-{[(4-{3,8- diazabicyclo[3.2.1]octan- 3-yl}-7-(8- ethylnaphthalen-1-yl)-8- fluoropyrido[4,3- d]pyrimidin-2- yl)oxy]methyl}- hexahydro-1H-pyrrolizin- 3-yl]methyl (3R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-3- |
| | | methylpiperazine-1- |
| | | carboxylate |
| 227 | 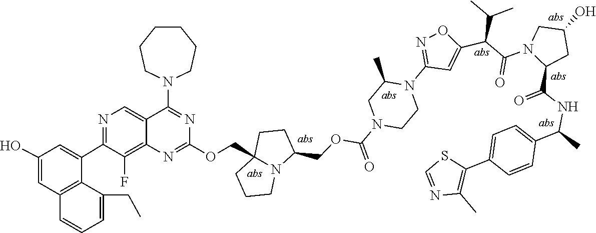 [SMILES: CCc1cccc2cc(O)cc(-c3ncc4c(N5CCCCCC5)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)[C@H](C)C5)CC6)nc4c3F)c12] | [(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl (3R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- 1,2-oxazol-3-yl}-3- |
| | | methylpiperazine-1- |
| | | carboxylate |
| 228 | 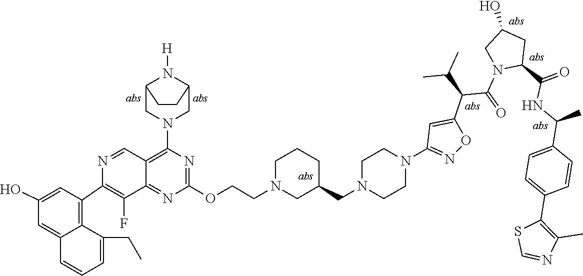 [SMILES: CCc1cccc2cc(O)cc(-c3ncc4c(N5CC6CCC(C5)N6)nc(OCCN5CCC[C@@H](CN6CCN(c7cc([C@H](C(=O)N8C[C@H](O)C[C@H]8C(=O)N[C@@H](C)c8ccc(-c9scnc9C)cc8)C(C)C)on7)CC6)C5)nc4c3F)c12] | (2S,4R)-1-[(2R)-2-[3-(4- {[(3S)-1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-3- yl]methyl}piperazin-1-yl)- 1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 229 | 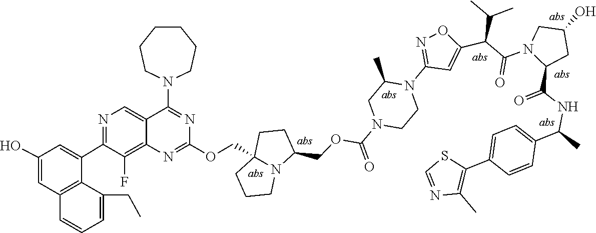 [SMILES: CCc1cccc2cc(O)cc(-c3ncc4c(N5CCCCCC5)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)[C@H](C)C5)CC6)nc4c3F)c12] | [(3S,7aS)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl (3R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- 1,2-oxazol-3-yl}-3- |
| | | methylpiperazine-1- |
| | | carboxylate |
| 230 | 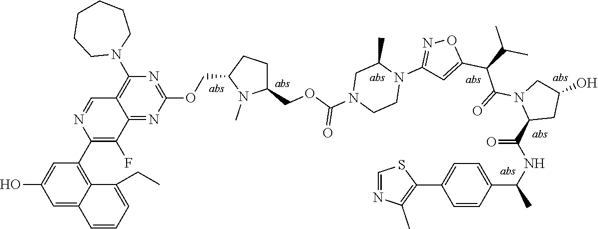 [SMILES: CCc1cccc2cc(O)cc(-c3ncc4c(N5CCCCCC5)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)[C@H](C)C6)N5C)nc4c3F)c12] | [(2S,5S)-5-({[4-(azepan- 1-yl)-7-(8-ethyl-3- hydroxynaphthalen-1-yl)- 8-fluoropyrido[4,3- d]pyrimidin-2- yl]oxy}methyl)-1- methylpyrrolidin-2- yl]methyl (3R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- 1,2-oxazol-3-yl}-3- |
| | | methylpiperazine-1- |
| | | carboxylate |
| 231 | 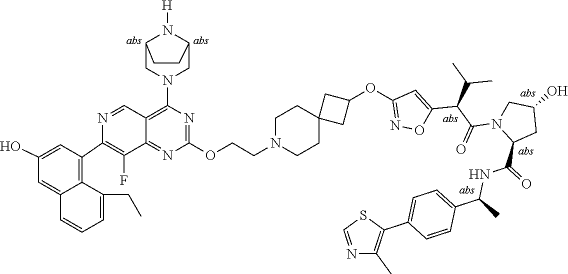 [SMILES: CCc1cccc2cc(O)cc(-c3ncc4c(N5CC6CCC(C5)N6)nc(OCCN5CCC6(CC5)CC(Oc5cc([C@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2R)-2-{3-[(7- {2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-7- azaspiro[3.5]nonan-2- yl)oxy]-1,2-oxazol-5-yl}- 3-methylbutanoyl]-4- hydroxy-N-[(1S)-1-[4-(4- methyl-1,3-thiazol-5- yl)phenyl]ethyl] pyrrolidine- 2-carboxamide |
| 232 | 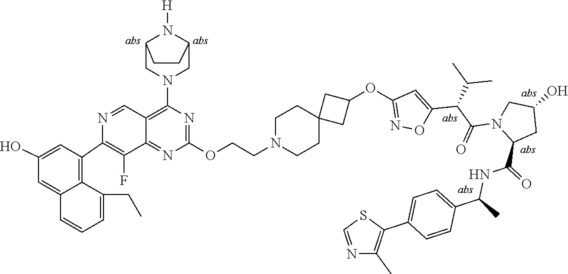 [SMILES: CCc1cccc2cc(O)cc(-c3ncc4c(N5CC6CCC(C5)N6)nc(OCCN5CCC6(CC5)CC(Oc5cc([C@@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2S)-2-{3-[(7- {2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-7- azaspiro[3.5]nonan-2- yl)oxy]-1,2-oxazol-5-yl}- 3-methylbutanoyl]-4- hydroxy-N-[(1S)-1-[4-(4- methyl-1,3-thiazol-5- yl)phenyl]ethyl] pyrrolidine- 2-carboxamide |
| 233 | 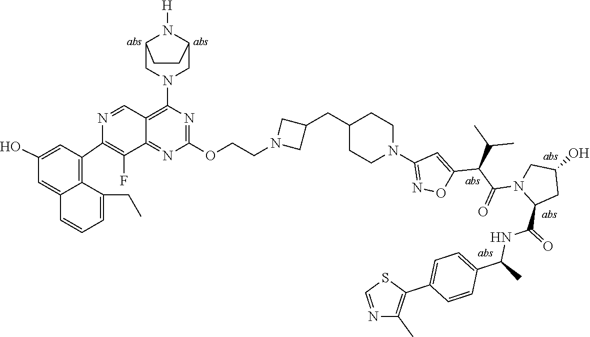 [SMILES: CCc1cccc2cc(O)cc(-c3ncc4c(N5CC6CCC(C5)N6)nc(OCCN5CC(CC6CCN(c7cc([C@H](C(=O)N8C[C@H](O)C[C@H]8C(=O)N[C@@H](C)c8ccc(-c9scnc9C)cc8)C(C)C)on7)CC6)C5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}azetidin-3- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 234 | 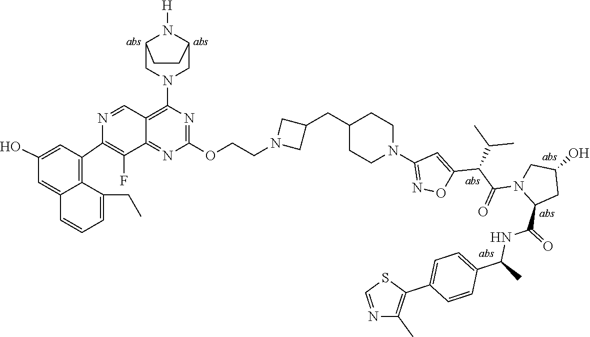 [SMILES: CCc1cccc2cc(O)cc(-c3ncc4c(N5CC6CCC(C5)N6)nc(OCCN5CC(CC6CCN(c7cc([C@@H](C(=O)N8C[C@H](O)C[C@H]8C(=O)N[C@@H](C)c8ccc(-c9scnc9C)cc8)C(C)C)on7)CC6)C5)nc4c3F)c12] | (2S,4R)-1-[(2S)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}azetidin-3- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 235 | 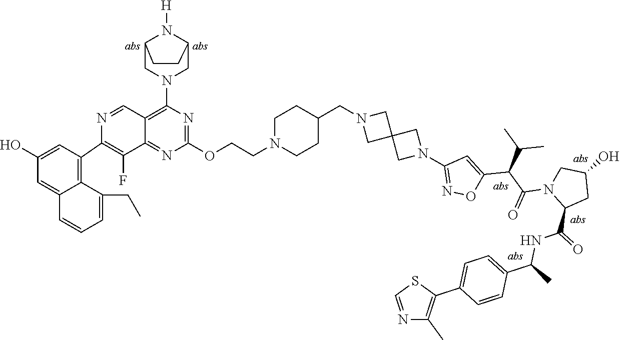 [SMILES: CCc1cccc2cc(O)cc(-c3ncc4c(N5CC6CCC(C5)N6)nc(OCCN5CCC(CN6CC7(C6)CN(c6cc([C@H](C(=O)N8C[C@H](O)C[C@H]8C(=O)N[C@@H](C)c8ccc(-c9scnc9C)cc8)C(C)C)on6)C7)CC5)nc4c3F)c12] | (2S,4R)-1-[(2R)-2-(3-{6- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]-2,6- diazaspiro[3.3]heptan-2- yl}-1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 236 | 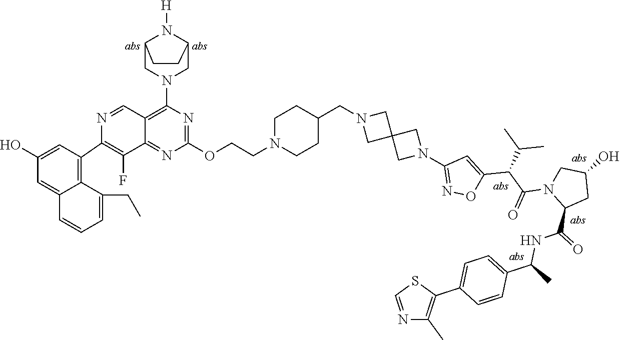 [SMILES: CCc1cccc2cc(O)cc(-c3ncc4c(N5CC6CCC(C5)N6)nc(OCCN5CCC(CN6CC7(C6)CN(c6cc([C@@H](C(=O)N8C[C@H](O)C[C@H]8C(=O)N[C@@H](C)c8ccc(-c9scnc9C)cc8)C(C)C)on6)C7)CC5)nc4c3F)c12] | (2S,4R)-1-[(2S)-2-(3-{6- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]-2,6- diazaspiro[3.3]heptan-2- yl}-1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 237 | 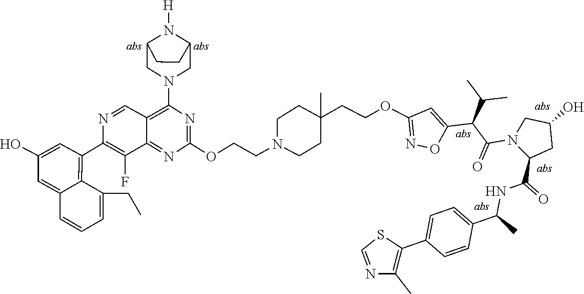 [SMILES: CCc1cccc2cc(O)cc(-c3ncc4c(N5CC6CCC(C5)N6)nc(OCCN5CCC(C)(CCOc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[2- (1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- methylpiperidin-4- yl)ethoxy]-1,2-oxazol-5- yl}-3-methylbutanoyl]-4- hydroxy-N-[(1S)-1-[4-(4- methyl-1,3-thiazol-5- yl)phenyl]ethyl] pyrrolidine- 2-carboxamide |
| 238 | 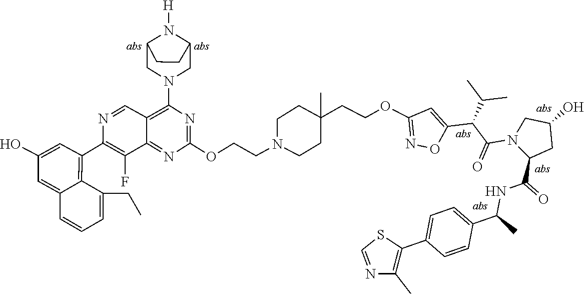 [SMILES: CCc1cccc2cc(O)cc(-c3ncc4c(N5CC6CCC(C5)N6)nc(OCCN5CCC(C)(CCOc6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2S)-2-{3-[2- (1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- methylpiperidin-4- yl)ethoxy]-1,2-oxazol-5- yl}-3-methylbutanoyl]-4- hydroxy-N-[(1S)-1-[4-(4- methyl-1,3-thiazol-5- yl)phenyl]ethyl] pyrrolidine- 2-carboxamide |
| 239 | 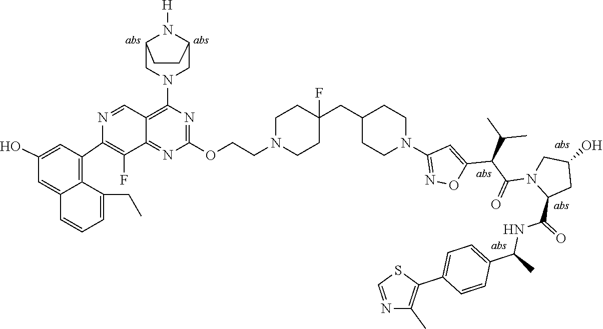 [SMILES: CCc1cccc2cc(O)cc(-c3ncc4c(N5CC6CCC(C5)N6)nc(OCCN5CCC(F)(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 240 | 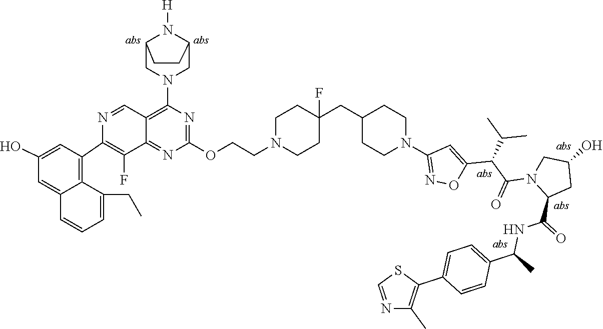 [SMILES: CCc1cccc2cc(O)cc(-c3ncc4c(N5CC6CCC(C5)N6)nc(OCCN5CCC(F)(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2S)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 241 | 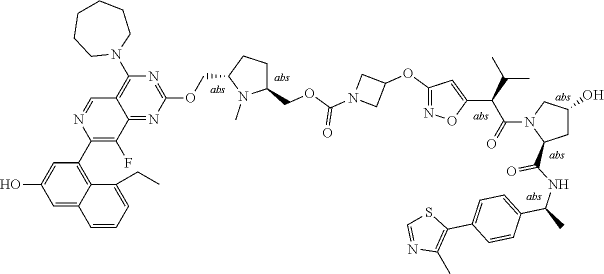 [SMILES: CCc1cccc2cc(O)cc(-c3ncc4c(N5CCCCCC5)nc(OC[C@@H]5CC[C@@H](COC(=O)N6CC(Oc7cc([C@H](C(=O)N8C[C@H](O)C[C@H]8C(=O)N[C@@H](C)c8ccc(-c9scnc9C)cc8)C(C)C)on7)C6)N5C)nc4c3F)c12] | [(2S,5S)-5-({[4-(azepan- 1-yl)-7-(8-ethyl-3- hydroxynaphthalen-1-yl)- 8-fluoropyrido[4,3- d]pyrimidin-2- yl]oxy}methyl)-1- methylpyrrolidin-2- yl]methyl 3-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- yl}oxy)azetidine-1- carboxylate |
| 242 | 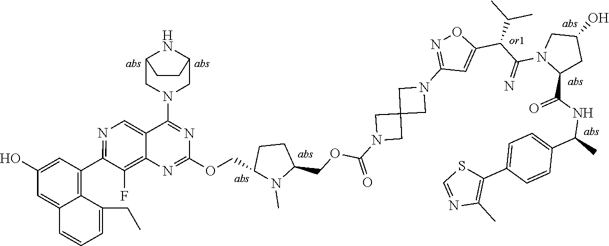 [SMILES: CCc1cccc2cc(O)cc(-c3ncc4c(N5CC6CCC(C5)N6)nc(OC[C@@H]5CC[C@@H](COC(=O)N6CC7(C6)CN(c6cc([C@@H](C(=N)N8C[C@H](O)C[C@H]8C(=O)N[C@@H](C)c8ccc(-c9scnc9C)cc8)C(C)C)on6)C7)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 6-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3-yl}-2,6- |
| | | diazaspiro[3.3]heptane- |
| | | 2-carboxylate |
| 243 | 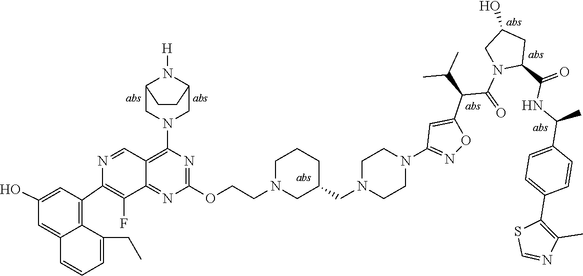 [SMILES: CCc1cccc2cc(O)cc(-c3ncc4c(N5CC6CCC(C5)N6)nc(OCCN5CCC[C@H](CN6CCN(c7cc([C@H](C(=O)N8C[C@H](O)C[C@H]8C(=O)N[C@@H](C)c8ccc(-c9scnc9C)cc8)C(C)C)on7)CC6)C5)nc4c3F)c12] | (2S,4R)-1-[(2R)-2-[3-(4- {[(3R)-1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-3- yl]methyl}piperazin-1-yl)- 1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 244 | 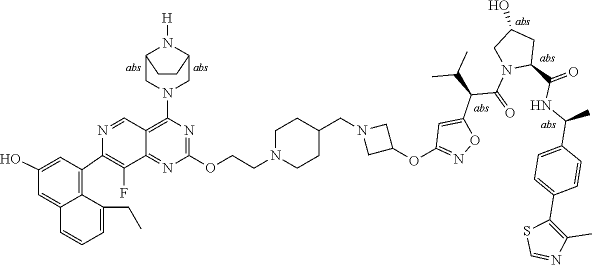 [SMILES: CCc1cccc2cc(O)cc(-c3ncc4c(N5CC6CCC(C5)N6)nc(OCCN5CCC(CN6CC(Oc7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-[3-({1- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-3- yl}oxy)-1,2-oxazol-5-yl]- 3-methylbutanoyl]-4- hydroxy-N-[(1S)-1-[4-(4- methyl-1,3-thiazol-5- yl)phenyl]ethyl] pyrrolidine- 2-carboxamide |
| 245 | 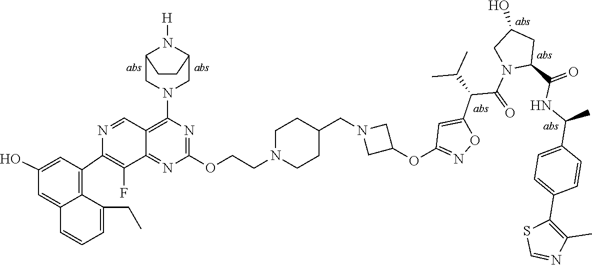 [SMILES: CCc1cccc2cc(O)cc(-c3ncc4c(N5CC6CCC(C5)N6)nc(OCCN5CCC(CN6CC(Oc7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2S)-2-[3-({1- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-3- yl}oxy)-1,2-oxazol-5-yl]- 3-methylbutanoyl]-4- hydroxy-N-[(1S)-1-[4-(4- methyl-1,3-thiazol-5- yl)phenyl]ethyl] pyrrolidine- 2-carboxamide |
| 246 | 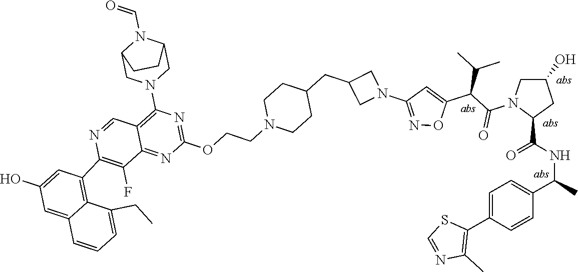 [SMILES: CCc1cccc2cc(O)cc(-c3ncc4c(N5CC6CCC(C5)N6C=O)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R)-2-[3-(3- {[1-(2-{[7-(8-ethyl-3- hydroxynaphthalen-1-yl)- 8-fluoro-4-{8-formyl-3,8- diazabicyclo[3.2.1]octan- 3-yl}pyrido[4,3- d]pyrimidin-2- yl]oxy}ethyl)piperidin-4- yl]methyl}azetidin-1-yl)- 1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 247 | 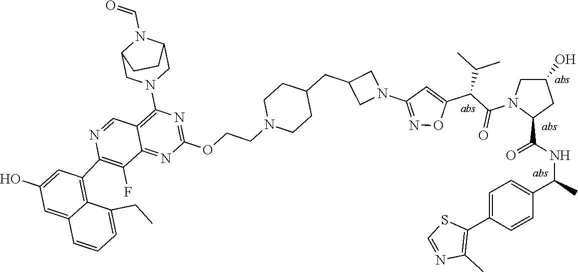 [SMILES: CCc1cccc2cc(O)cc(-c3ncc4c(N5CC6CCC(C5)N6C=O)nc(OCCN5CCC(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2S)-2-[3-(3- {[1-(2-{[7-(8-ethyl-3- hydroxynaphthalen-1-yl)- 8-fluoro-4-{8-formyl-3,8- diazabicyclo[3.2.1]octan- 3-yl}pyrido[4,3- d]pyrimidin-2- yl]oxy}ethyl)piperidin-4- yl]methyl}azetidin-1-yl)- 1,2-oxazol-5-yl]-3- methylbutanoyl]-4- hydroxy-N-[(1S)-1-[4-(4- methyl-1,3-thiazol-5- yl)phenyl]ethyl] pyrrolidine- 2-carboxamide |
| 248 | 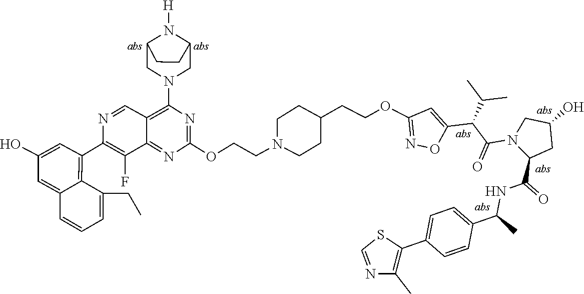 [SMILES: CCc1cccc2cc(O)cc(-c3ncc4c(N5CC6CCC(C5)N6)nc(OCCN5CCC(CCOc6cc([C@@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2S)-2-{3-[2- (1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)ethoxy]-1,2-oxazol-5- yl}-3-methylbutanoyl]-4- hydroxy-N-[(1S)-1-[4-(4- methyl-1,3-thiazol-5- yl)phenyl]ethyl] pyrrolidine- 2-carboxamide |
| 249 | 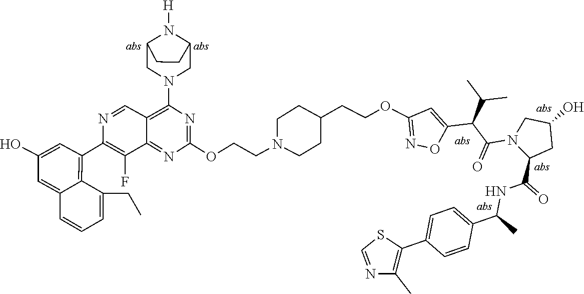 [SMILES: CCc1cccc2cc(O)cc(-c3ncc4c(N5CC6CCC(C5)N6)nc(OCCN5CCC(CCOc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[2- (1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)ethoxy]-1,2-oxazol-5- yl}-3-methylbutanoyl]-4- hydroxy-N-[(1S)-1-[4-(4- methyl-1,3-thiazol-5- yl)phenyl]ethyl] pyrrolidine- 2-carboxamide |
| 250 | 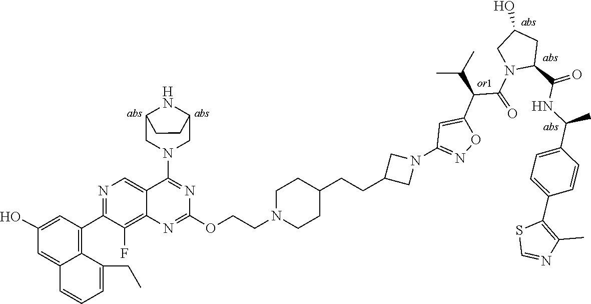 [SMILES: CCc1cccc2cc(O)cc(-c3ncc4c(N5CC6CCC(C5)N6)nc(OCCN5CCC(CCC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R*)-2-(3-{3- [2-(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)ethyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 251 | 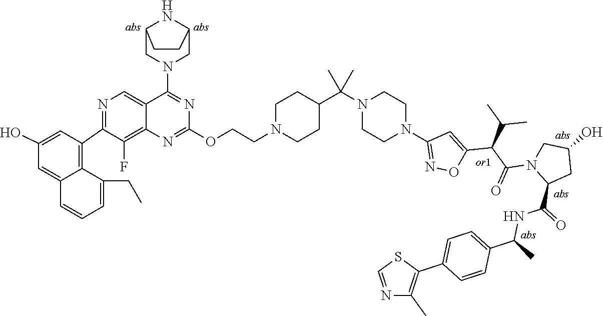 [SMILES: CCc1cccc2cc(O)cc(-c3ncc4c(N5CC6CCC(C5)N6)nc(OCCN5CCC(C(C)(C)N6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R*)-2-(3-{4- [2-(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)propan-2-yl]piperazin- 1-yl}-1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 252 | 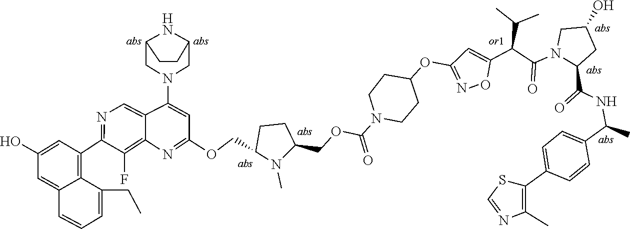 [SMILES: CCc1cccc2cc(O)cc(-c3ncc4c(N5CC6CCC(C5)N6)cc(OC[C@@H]5CC[C@@H](COC(=O)N6CCC(Oc7cc([C@H](C(=O)N8C[C@H](O)C[C@H]8C(=O)N[C@@H](C)c8ccc(-c9scnc9C)cc8)C(C)C)on7)CC6)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 4-({5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}oxy)piperidine-1- |
| | | carboxylate |
| 253 | 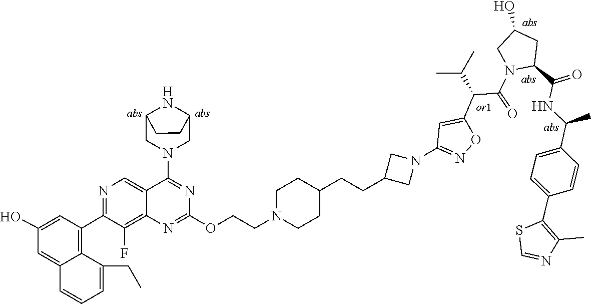 [SMILES: CCc1cccc2cc(O)cc(-c3ncc4c(N5CC6CCC(C5)N6)nc(OCCN5CCC(CCC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R*)-2-(3-{3- [2-(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)ethyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 254 | 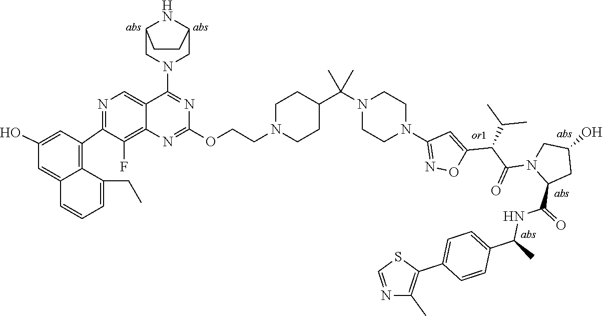 [SMILES: CCc1cccc2cc(O)cc(-c3ncc4c(N5CC6CCC(C5)N6)nc(OCCN5CCC(C(C)(C)N6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R*)-2-(3-{4- [2-(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)propan-2-yl]piperazin- 1-yl}-1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 255 | 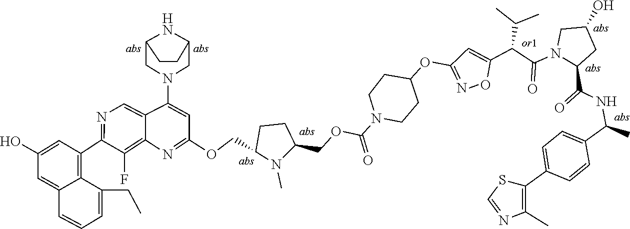 [SMILES: CCc1cccc2cc(O)cc(-c3ncc4c(N5CC6CCC(C5)N6)cc(OC[C@@H]5CC[C@@H](COC(=O)N6CCC(Oc7cc([C@@H](C(=O)N8C[C@H](O)C[C@H]8C(=O)N[C@@H](C)c8ccc(-c9scnc9C)cc8)C(C)C)on7)CC6)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 4-({5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}oxy)piperidine-1- |
| | | carboxylate |
| 256 | 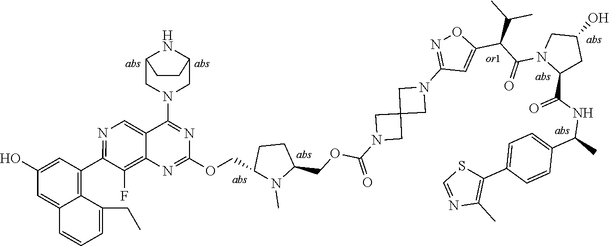 [SMILES: CCc1cccc2cc(O)cc(-c3ncc4c(N5CC6CCC(C5)N6)nc(OC[C@@H]5CC[C@@H](COC(=O)N6CC7(C6)CN(c6cc([C@H](C(=O)N8C[C@H](O)C[C@H]8C(=O)N[C@@H](C)c8ccc(-c9scnc9C)cc8)C(C)C)on6)C7)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 6-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3-yl}-2,6- |
| | | diazaspiro[3.3]heptane- |
| | | 2-carboxylate |
| 257 | 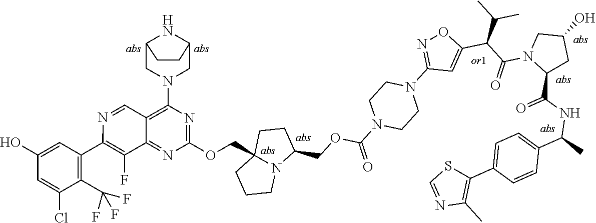 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cnc(-c8cc(O)cc(Cl)c8C(F)(F)F)c(F)c7n6)CCCN45)CC3)no2)C(C)C)cc1] | [(3S,7aR)-7a-[({7-[3- chloro-5-hydroxy-2- (trifluoromethyl)phenyl]- 4-{3,8- diazabicyclo[3.2.1]octan- 3-yl}-8-fluoropyrido[4,3- d]pyrimidin-2- yl}oxy)methyl]- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- |
| | | methyl-1-oxobutan-2-yl]- |
| | | 1,2-oxazol-3- |
| | | yl}piperazine-1- |
| | | carboxylate |
| 258 | 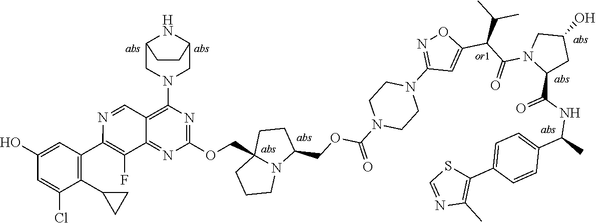 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cnc(-c8cc(O)cc(Cl)c8C8CC8)c(F)c7n6)CCCN45)CC3)no2)C(C)C)cc1] | [(3S,7aR)-7a-({[7-(3- chloro-2-cyclopropyl-5- hydroxyphenyl)-4-{3,8- diazabicyclo[3.2.1]octan- 3-yl}-8-fluoropyrido[4,3- d]pyrimidin-2- yl]oxy}methyl)- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 259 | 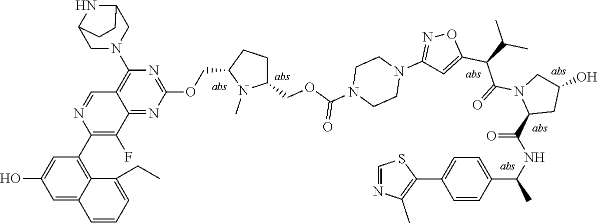 [SMILES: CCc1cccc2cc(O)cc(-c3ncc4c(N5CC6CCC(C5)N6)nc(OC[C@@H]5CC[C@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12] | [(2R,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 4-{5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 260 | 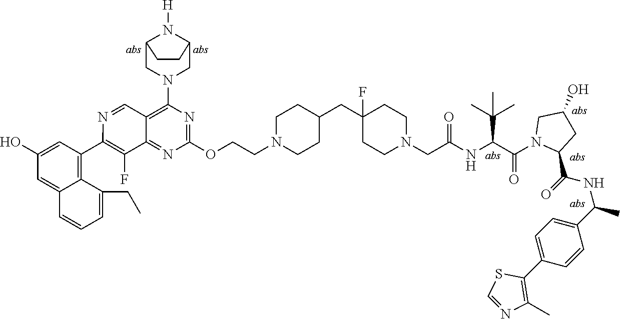 [SMILES: CCc1cccc2cc(O)cc(-c3ncc4c(N5CC6CCC(C5)N6)nc(OCCN5CCC(CC6(F)CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12] | (2S,4R)-1-[(2S)-2-(2-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]-4- fluoropiperidin-1- yl}acetamido)-3,3- dimethylbutanoyl]-4- hydroxy-N-[(1S)-1-[4-(4- methyl-1,3-thiazol-5- yl)phenyl]ethyl] pyrrolidine- 2-carboxamide |
| 261 | 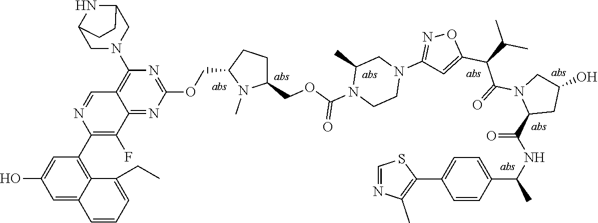 [SMILES: CCc1cccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)C[C@@H]6C)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl (2S)-4-{5-[(2R)- 1-[(2S,4R)-4-hydroxy-2- {[(1S)-1-[4-(4-methyl- 1,3-thiazol-5- yl)phenyl]ethyl]carbamoyl} pyrrolidin-1-yl]-3- |
| | | methyl-1-oxobutan-2-yl]- |
| | | 1,2-oxazol-3-yl}-2- |
| | | methylpiperazine-1- |
| | | carboxylate |
| 262 | 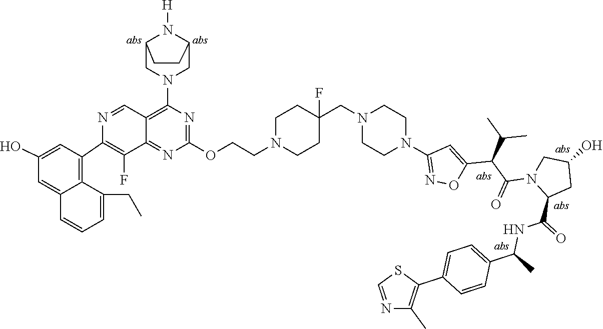 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methyl]piperazin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 263 | 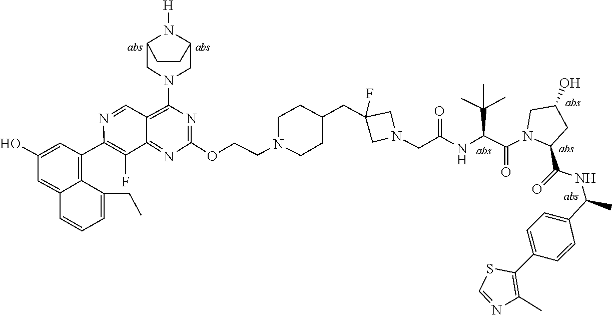 [SMILES: CCc1cccc2cc(O)cc(-c3ncc4c(N5CC6CCC(C5)N6)nc(OCCN5CCC(CC6(F)CN(CC(=O)N[C@H](C(=O)N7C[C@H](O)C[C@H]7C(=O)N[C@@H](C)c7ccc(-c8scnc8C)cc7)C(C)(C)C)C6)CC5)nc4c3F)c12] | (2S,4R)-1-[(2S)-2-(2-{3- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]-3- fluoroazetidin-1- yl}acetamido)-3,3- dimethylbutanoyl]-4- hydroxy-N-[(1S)-1-[4-(4- methyl-1,3-thiazol-5- yl)phenyl]ethyl] pyrrolidine- 2-carboxamide |
| 264 | 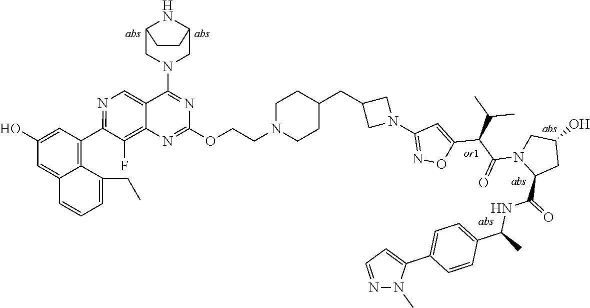 [SMILES: CCc1cccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9ccnn9C)cc8)C(C)C)on7)C6)CC5)nc4c3F)c12] | (2S,4R)-1-[(2R*)-2-(3-{3- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 265 | 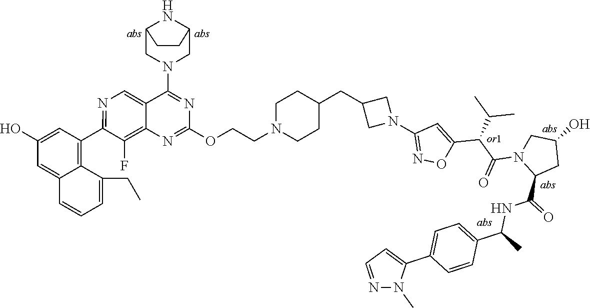 [SMILES: CCc1cccc2cc(O)cc(-c3ncc4c(N5CC6CCC(C5)N6)nc(OCCN5CCC(CC6CN(c7cc([C@@H](C(=O)N8C[C@H](O)C[C@H]8C(=O)N[C@@H](C)c8ccc(-c9ccnn9C)cc8)C(C)C)on7)C6)CC5)nc4c3F)c12] | (2S,4R)-1-[(2R*)-2-(3-{3- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(1- methyl-1H-pyrazol-5- yl)phenyl]ethyl] pyrrolidine- 2-carboxamide |
| 266 | 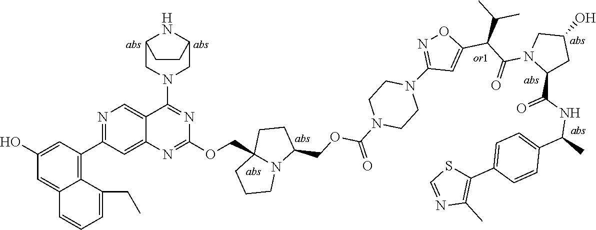 [SMILES: CCc1cccc2cc(O)cc(-c3cc4nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc(N5CC6CCC(C5)N6)c4cn3)c12] | [(3S,7aR)-7a-{[(4-{3,8- diazabicyclo[3.2.1]octan- 3-yl}-7-(8-ethyl-3- hydroxynaphthalen-1- yl)pyrido[4,3-d]pyrimidin- 2-yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- |
| | | yl}piperazine-1- |
| | | carboxylate |
| 267 | 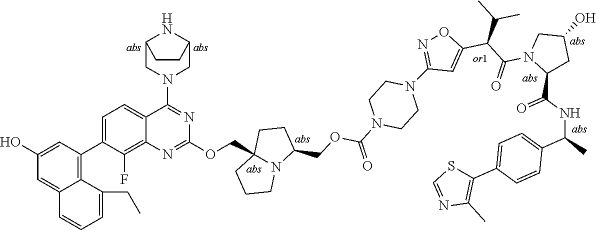 [SMILES: CCc1cccc2cc(O)cc(-c3ccc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-{[(4-{3,8- diazabicyclo[3.2.1]octan- 3-yl}-7-(8-ethyl-3- hydroxynaphthalen-1-yl)- 8-fluoroquinazolin-2- yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- |
| | | yl}piperazine-1- |
| | | carboxylate |
| 268 | 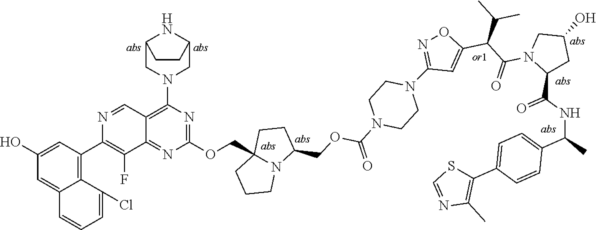 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cnc(-c8cc(O)cc9cccc(Cl)c89)c(F)c7n6)CCCN45)CC3)no2)C(C)C)cc1] | [(3S,7aR)-7a-({[7-(8- chloro-3- hydroxynaphthalen-1-yl)- 4-{3,8- diazabicyclo[3.2.1]octan- 3-yl}-8-fluoropyrido[4,3- d]pyrimidin-2- yl]oxy}methyl)- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- |
| | | methyl-1-oxobutan-2-yl]- |
| | | 1,2-oxazol-3- |
| | | yl}piperazine-1- |
| | | carboxylate |
| 269 | 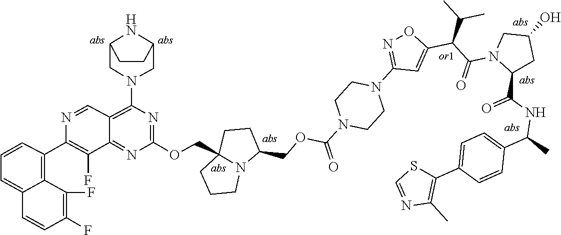 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cnc(-c8cccc9ccc(F)c(F)c89)c(F)c7n6)CCCN45)CC3)no2)C(C)C)cc1] | [(3S,7aR)-7a-{[(4-{3,8- diazabicyclo[3.2.1]octan- 3-yl}-7-(7,8- difluoronaphthalen-1-yl)- 8-fluoropyrido[4,3- d]pyrimidin-2- yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 270 | 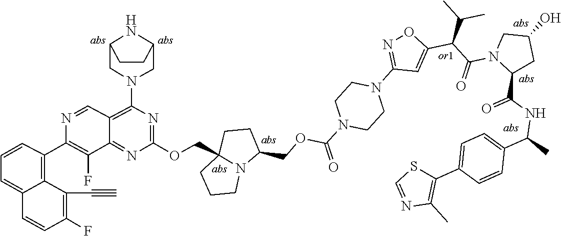 [SMILES: C#Cc1c(F)ccc2cccc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-{[(4-{3,8- diazabicyclo[3.2.1]octan- 3-yl}-7-(8-ethynyl-7- fluoronaphthalen-1-yl)-8- fluoropyrido[4,3- d]pyrimidin-2- yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 271 | 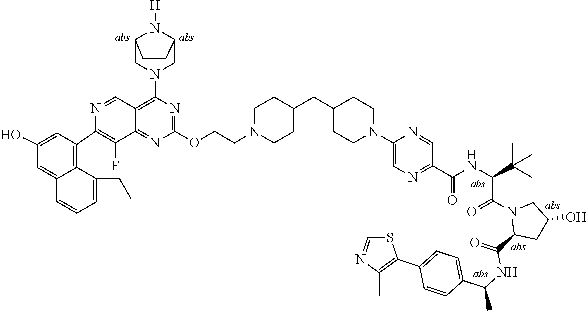 [SMILES: CCc1cccc2cc(O)cc(-c3ncc4c(N5CC6CCC(C5)N6)nc(OCCN5CCC(CC6CCN(c7cnc(C(=O)N[C@H](C(=O)N8C[C@H](O)C[C@H]8C(=O)N[C@@H](C)c8ccc(-c9scnc9C)cc8)C(C)(C)C)cn7)CC6)CC5)nc4c3F)c12] | 5-{4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperidin-1-yl}- N-[(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3,3- dimethyl-1-oxobutan-2- yl]pyrazine-2- carboxamide |
| 272 | 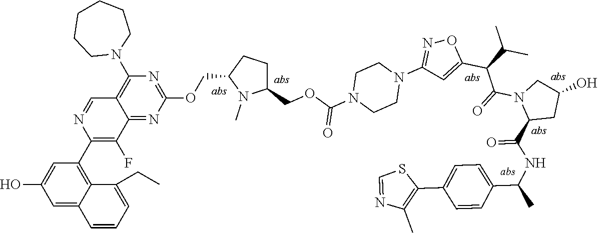 [SMILES: CCc1cccc2cc(O)cc(-c3ncc4c(N5CCCCCC5)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12] | [(2S,5S)-5-({[4-(azepan- 1-yl)-7-(8-ethyl-3- hydroxynaphthalen-1-yl)- 8-fluoropyrido[4,3- d]pyrimidin-2- yl]oxy}methyl)-1- methylpyrrolidin-2- yl]methyl 4-{5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- |
| | | yl}piperazine-1- |
| | | carboxylate |
| 273 | 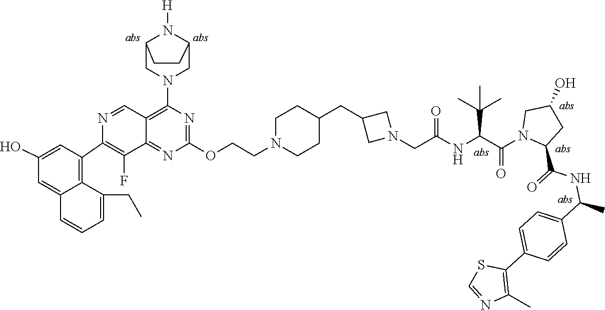 [SMILES: CCc1cccc2cc(O)cc(-c3ncc4c(N5CC6CCC(C5)N6)nc(OCCN5CCC(CC6CN(CC(=O)N[C@H](C(=O)N7C[C@H](O)C[C@H]7C(=O)N[C@@H](C)c7ccc(-c8scnc8C)cc7)C(C)(C)C)C6)CC5)nc4c3F)c12] | (2S,4R)-1-[(2S)-2-(2-{3- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-1- yl}acetamido)-3,3- dimethylbutanoyl]-4- hydroxy-N-[(1S)-1-[4-(4- methyl-1,3-thiazol-5- yl)phenyl]ethyl] pyrrolidine- 2-carboxamide |
| 274 | 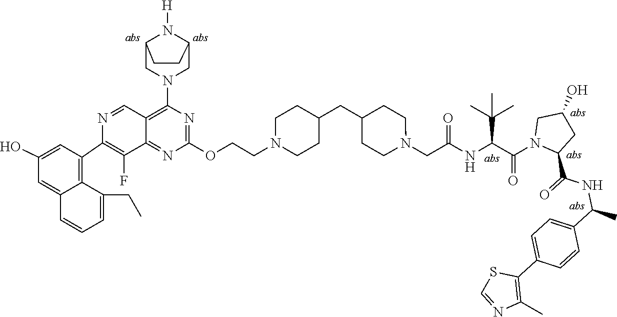 [SMILES: CCc1cccc2cc(O)cc(-c3ncc4c(N5CC6CCC(C5)N6)nc(OCCN5CCC(CC6CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12] | (2S,4R)-1-[(2S)-2-(2-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperidin-1- yl}acetamido)-3,3- dimethylbutanoyl]-4- hydroxy-N-[(1S)-1-[4-(4- methyl-1,3-thiazol-5- yl)phenyl]ethyl] pyrrolidine- 2-carboxamide |
| 275 | 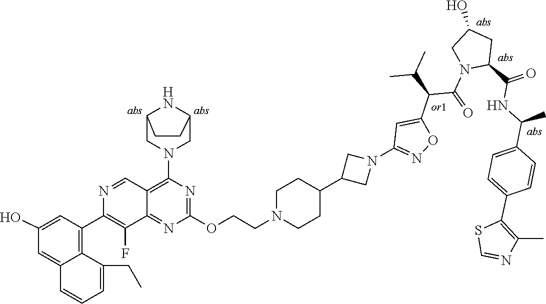 [SMILES: CCc1cccc2cc(O)cc(-c3ncc4c(N5CC6CCC(C5)N6)nc(OCCN5CCC(C6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R*)-2-{3-[3- (1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)azetidin-1-yl]-1,2- oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 276 | 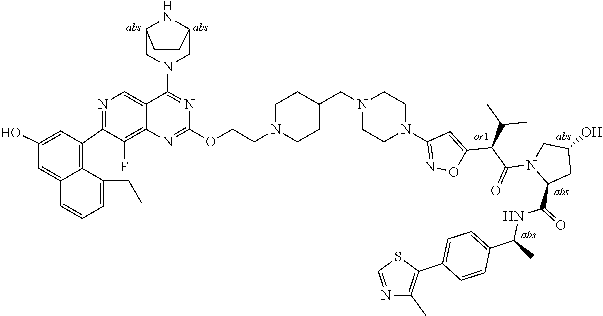 [SMILES: CCc1cccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R*)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperazin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 277 | 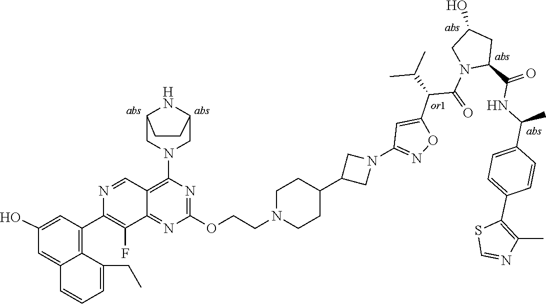 [SMILES: CCc1cccc2cc(O)cc(-c3ncc4c(N5CC6CCC(C5)N6)nc(OCCN5CCC(C6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R*)-2-{3-[3- (1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)azetidin-1-yl]-1,2- oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 278 | 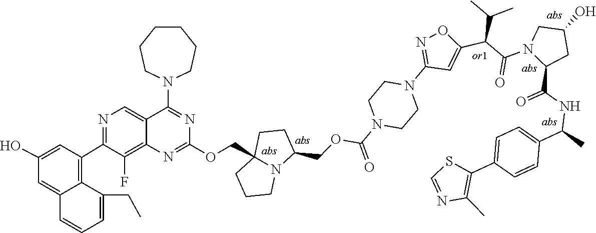 [SMILES: CCc1cccc2cc(O)cc(-c3ncc4c(N5CCCCCC5)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- |
| | | yl}piperazine-1- |
| | | carboxylate |
| 279 | 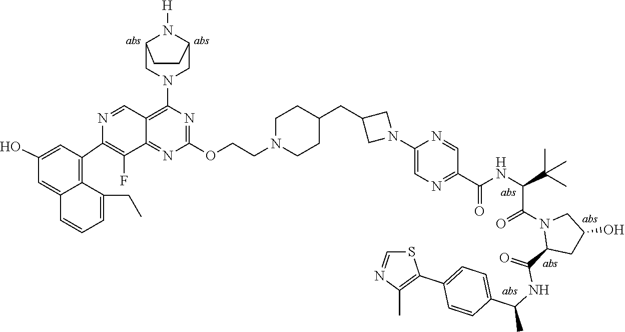 [SMILES: CCc1cccc2cc(O)cc(-c3ncc4c(N5CC6CCC(C5)N6)nc(OCCN5CCC(CC6CN(c7cnc(C(=O)N[C@H](C(=O)N8C[C@H](O)C[C@H]8C(=O)N[C@@H](C)c8ccc(-c9scnc9C)cc8)C(C)(C)C)cn7)C6)CC5)nc4c3F)c12] | 5-{3-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]azetidin-1-yl}- N-[(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3,3- dimethyl-1-oxobutan-2- yl]pyrazine-2- carboxamide |
| 280 | 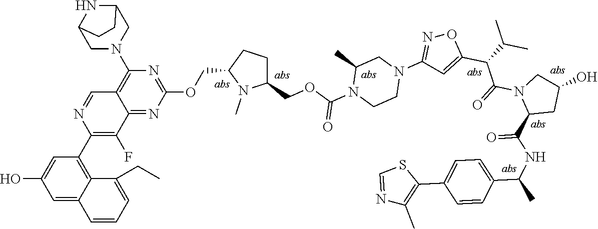 [SMILES: CCc1cccc2cc(O)cc(-c3ncc4c(N5CC6CCC(C5)N6)nc(OC[C@@H]5CC[C@@H](COC(=O)N6CCN(c7cc([C@@H](C(=O)N8C[C@H](O)C[C@H]8C(=O)N[C@@H](C)c8ccc(-c9scnc9C)cc8)C(C)C)on7)C[C@@H]6C)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl (2S)-4-{5-[(2S)- 1-[(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3-yl}-2- |
| | | methylpiperazine-1- |
| | | carboxylate |
| 281 | 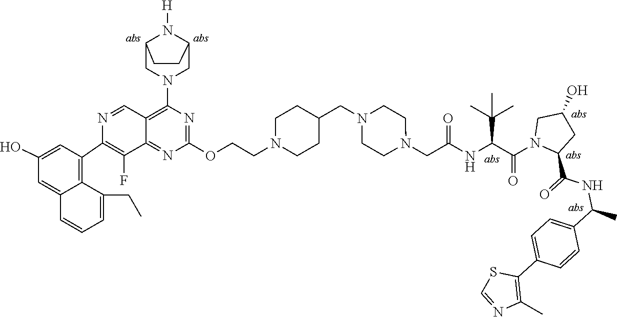 [SMILES: CCc1cccc2cc(O)cc(-c3ncc4c(N5CC6CCC(C5)N6)nc(OCCN5CCC(CN6CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12] | (2S,4R)-1-[(2S)-2-(2-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperazin-1- yl}acetamido)-3,3- dimethylbutanoyl]-4- hydroxy-N-[(1S)-1-[4-(4- methyl-1,3-thiazol-5- yl)phenyl]ethyl] pyrrolidine- 2-carboxamide |
| 282 | 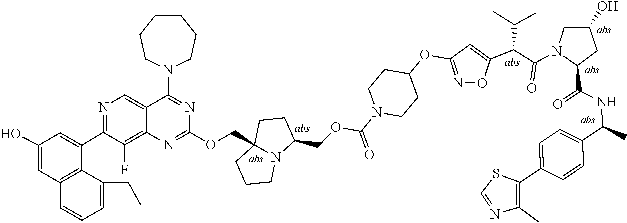 [SMILES: CCc1cccc2cc(O)cc(-c3ncc4c(N5CCCCCC5)nc(OC[C@]56CCCN5[C@H](COC(=O)N5CCC(Oc7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl 4-({5-[(2S)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- |
| | | yl}oxy)piperidine-1- |
| | | carboxylate |
| 283 | 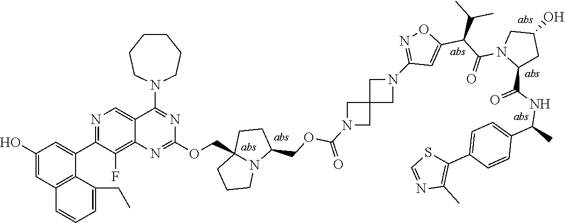 [SMILES: CCc1cccc2cc(O)cc(-c3ncc4c(N5CCCCCC5)nc(OC[C@]56CCCN5[C@H](COC(=O)N5CC7(C5)CN(c5cc([C@H](C(=O)N8C[C@H](O)C[C@H]8C(=O)N[C@@H](C)c8ccc(-c9scnc9C)cc8)C(C)C)on5)C7)CC6)nc4c3F)c12] | [(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl 6-{5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3-yl}-2,6- |
| | | diazaspiro[3.3]heptane- |
| | | 2-carboxylate |
| 284 | 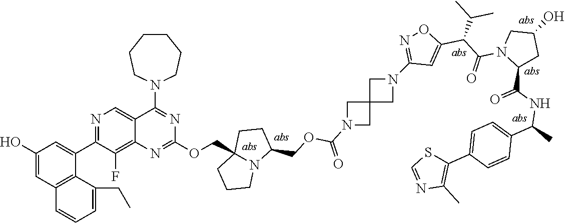 [SMILES: CCc1cccc2cc(O)cc(-c3ncc4c(N5CCCCCC5)nc(OC[C@]56CCCN5[C@H](COC(=O)N5CC7(C5)CN(c5cc([C@@H](C(=O)N8C[C@H](O)C[C@H]8C(=O)N[C@@H](C)c8ccc(-c9scnc9C)cc8)C(C)C)on5)C7)CC6)nc4c3F)c12] | [(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl 6-{5-[(2S)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3-yl}-2,6- |
| | | diazaspiro[3.3]heptane- |
| | | 2-carboxylate |
| 285 | 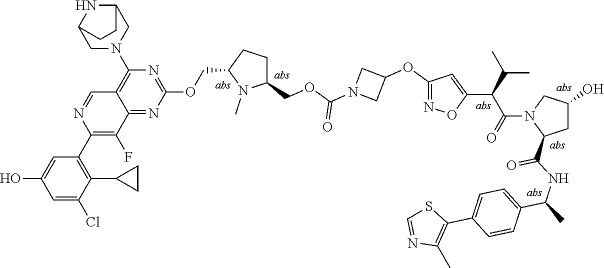 | [(2S,5S)-5-({[7-(3-chloro- 2-cyclopropyl-5- hydroxyphenyl)-4-{3,8- diazabicyclo[3.2.1]octan- 3-yl}-8-fluoropyrido[4,3- d]pyrimidin-2- yl]oxy}methyl)-1- methylpyrrolidin-2- yl]methyl 3-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- yl}oxy)azetidine-1- |
| | | carboxylate |
| 286 | 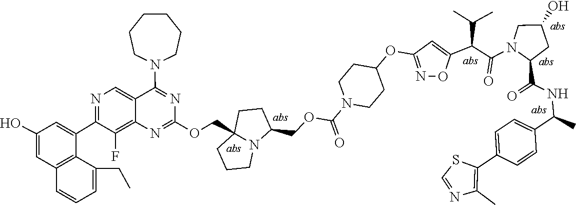 [SMILES: CCc1cccc2cc(O)cc(-c3ncc4c(N5CCCCCC5)nc(OC[C@]56CCCN5[C@H](COC(=O)N5CCC(Oc7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl 4-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}oxy)piperidine-1- |
| | | carboxylate |
| 287 | 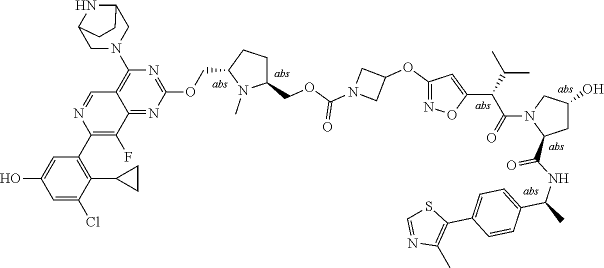 | [(2S,5S)-5-({[7-(3-chloro- 2-cyclopropyl-5- hydroxyphenyl)-4-{3,8- diazabicyclo[3.2.1]octan- 3-yl}-8-fluoropyrido[4,3- d]pyrimidin-2- yl]oxy}methyl)-1- methylpyrrolidin-2- yl]methyl 3-({5-[(2S)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- yl}oxy)azetidine-1- |
| | | carboxylate |
| 288 | 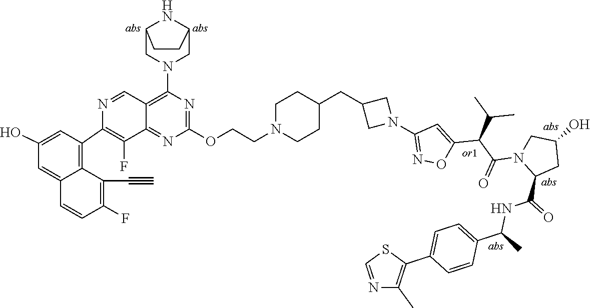 [SMILES: C#Cc1c(F)ccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12] | (2S,4R)-1-[(2R*)-2-(3-{3- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]azetidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 289 | 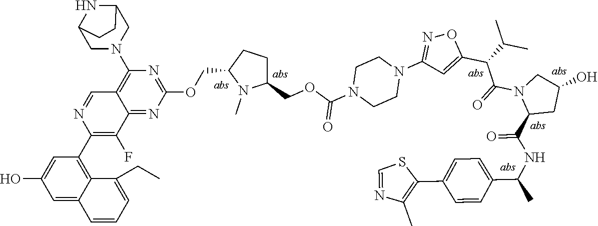 [SMILES: CCc1cccc2cc(O)cc(-c3ncc4c(N5CC6CCC(C5)N6)nc(OC[C@@H]5CC[C@@H](COC(=O)N6CCN(c7cc([C@@H](C(=O)N8C[C@H](O)C[C@H]8C(=O)N[C@@H](C)c8ccc(-c9scnc9C)cc8)C(C)C)on7)CC6)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 4-{5-[(2S)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 290 | 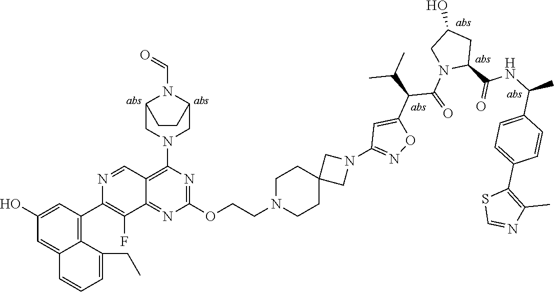 [SMILES: CCc1cccc2cc(O)cc(-c3ncc4c(N5CC6CCC(C5)N6C=O)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12] | (2S,4R)-1-[(2R)-2-{3-[7- (2-{[7-(8-ethyl-3- hydroxynaphthalen-1-yl)- 8-fluoro-4-{8-formyl-3,8- diazabicyclo[3.2.1]octan- 3-yl}pyrido[4,3- d]pyrimidin-2- yl]oxy}ethyl)-2,7- diazaspiro[3.5]nonan-2- yl]-1,2-oxazol-5-yl}-3- methylbutanoyl]-4- hydroxy-N-[(1S)-1-[4-(4- methyl-1,3-thiazol-5- yl)phenyl]ethyl] pyrrolidine- 2-carboxamide |
| 291 | 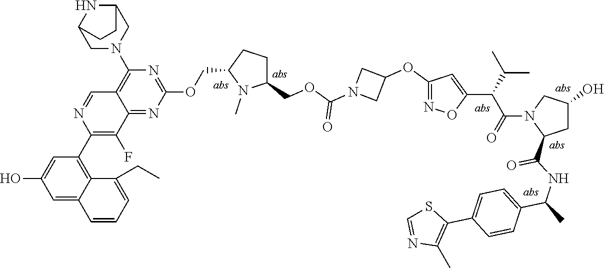 | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 3-({5-[(2S)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- yl}oxy)azetidine-1- |
| | | carboxylate |
| 292 | 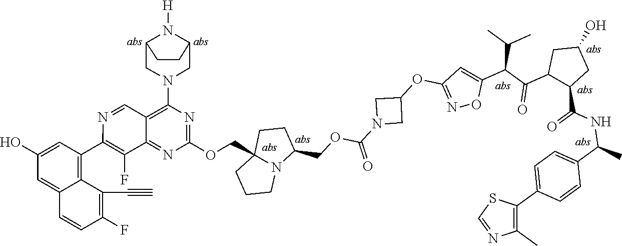 [SMILES: C#Cc1c(F)ccc2cc(O)cc(-c3ncc4c(N5CC6CCC(C5)N6)nc(OC[C@]56CCCN5[C@H](COC(=O)N5CC(Oc7cc([C@H](C(=O)C8C[C@H](O)C[C@H]8C(=O)N[C@@H](C)c8ccc(-c9scnc9C)cc8)C(C)C)on7)C5)CC6)nc4c3F)c12] | [(3S,7aR)-7a-{[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]methyl}- hexahydro-1H-pyrrolizin- 3-yl]methyl 3-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}oxy)azetidine-1- |
| | | carboxylate |
| 293 | 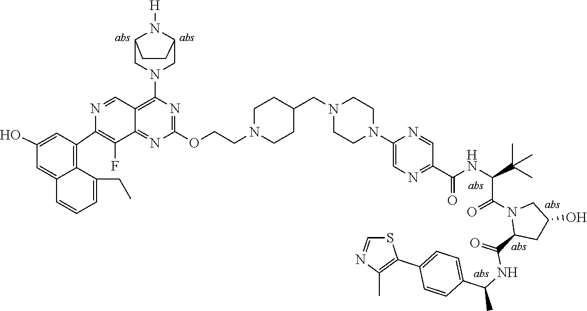 [SMILES: CCc1cccc2cc(O)cc(-c3ncc4c(N5CC6CCC(C5)N6)nc(OCCN5CCC(CN6CCN(c7cnc(C(=O)N[C@H](C(=O)N8C[C@H](O)C[C@H]8C(=O)N[C@@H](C)c8ccc(-c9scnc9C)cc8)C(C)(C)C)cn7)CC6)CC5)nc4c3F)c12] | 5-{4-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperazin-1-yl}- N-[(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3,3- dimethyl-1-oxobutan-2- yl]pyrazine-2- carboxamide |
| 294 | 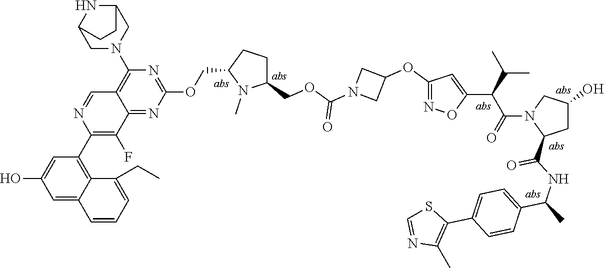 | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl 3-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- yl}oxy)azetidine-1- |
| | | carboxylate |
| 295 | 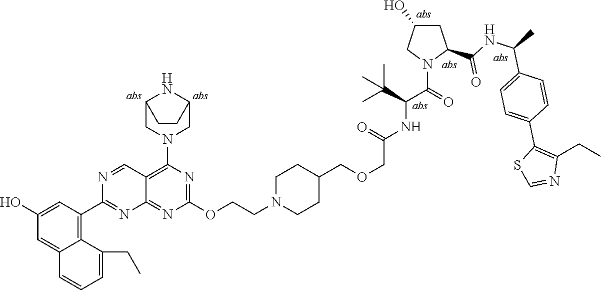 [SMILES: CCc1ncsc1-c1ccc([C@H](C)NC(=O)[C@@H]2C[C@@H](O)CN2C(=O)[C@@H](NC(=O)COCC2CCN(CCOc3nc(N4CC5CCC(C4)N5)c4cnc(-c5cc(O)cc6cccc(CC)c56)nc4n3)CC2)C(C)(C)C)cc1] | (2S,4R)-1-[(2S)-2-{2-[(1- {2-[(4-{3,8- diazabicyclo[3.2.1]octan- 3-yl}-7-(8-ethyl-3- hydroxynaphthalen-1- yl)pyrimido[4,5- d][1,3]diazin-2- yl)oxy]ethyl}piperidin-4- yl)methoxy]acetamido}- 3,3-dimethylbutanoyl]-N- [(1S)-1-[4-(4-ethyl-1,3- thiazol-5- yl)phenyl]ethyl]-4- hydroxypyrrolidine-2- carboxamide |
| 296 | 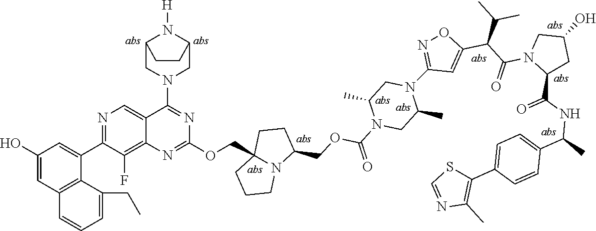 [SMILES: CCc1cccc2cc(O)cc(-c3ncc4c(N5CC6CCC(C5)N6)nc(OC[C@]56CCCN5[C@H](COC(=O)N5C[C@H](C)N(c7cc([C@H](C(=O)N8C[C@H](O)C[C@H]8C(=O)N[C@@H](C)c8ccc(-c9scnc9C)cc8)C(C)C)on7)C[C@H]5C)CC6)nc4c3F)c12] | [(3S,7aR)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2R,5S)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-2,5- |
| | | dimethylpiperazine-1- |
| | | carboxylate |
| 297 | 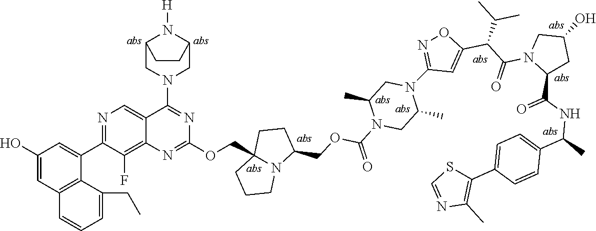 [SMILES: CCc1cccc2cc(O)cc(-c3ncc4c(N5CC6CCC(C5)N6)nc(OC[C@]56CCCN5[C@H](COC(=O)N5C[C@@H](C)N(c7cc([C@@H](C(=O)N8C[C@H](O)C[C@H]8C(=O)N[C@@H](C)c8ccc(-c9scnc9C)cc8)C(C)C)on7)C[C@@H]5C)CC6)nc4c3F)c12] | [(3S,7aR)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2S,5R)-4-{5- [(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-2,5- |
| | | dimethylpiperazine-1- |
| | | carboxylate |
| 298 | 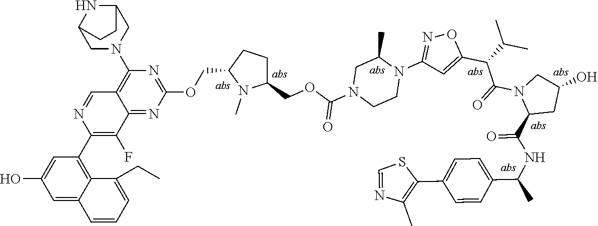 [SMILES: CCc1cccc2cc(O)cc(-c3ncc4c(N5CC6CCC(C5)N6)nc(OC[C@@H]5CC[C@@H](COC(=O)N6CCN(c7cc([C@@H](C(=O)N8C[C@H](O)C[C@H]8C(=O)N[C@@H](C)c8ccc(-c9scnc9C)cc8)C(C)C)on7)[C@H](C)C6)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl (3R)-4-{5-[(2S)- 1-[(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3-yl}-3- |
| | | methylpiperazine-1- |
| | | carboxylate |
| 299 | 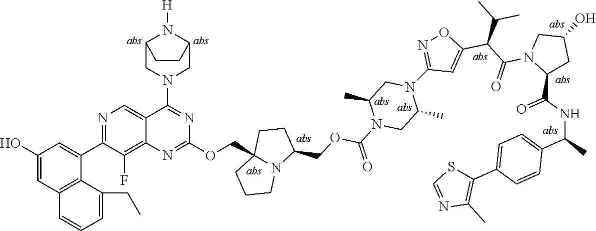 [SMILES: CCc1cccc2cc(O)cc(-c3ncc4c(N5CC6CCC(C5)N6)nc(OC[C@]56CCCN5[C@H](COC(=O)N5C[C@@H](C)N(c7cc([C@H](C(=O)N8C[C@H](O)C[C@H]8C(=O)N[C@@H](C)c8ccc(-c9scnc9C)cc8)C(C)C)on7)C[C@@H]5C)CC6)nc4c3F)c12] | [(3S,7aR)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2S,5R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-2,5- |
| | | dimethylpiperazine-1- |
| | | carboxylate |
| 300 | 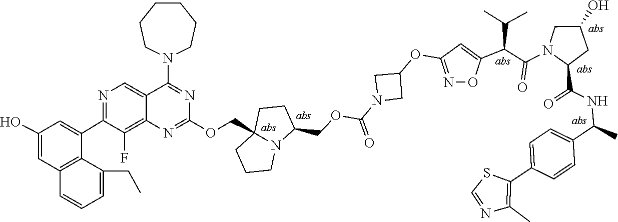 [SMILES: CCc1cccc2cc(O)cc(-c3ncc4c(N5CCCCCC5)nc(OC[C@]56CCCN5[C@H](COC(=O)N5CC(Oc7cc([C@H](C(=O)N8C[C@H](O)C[C@H]8C(=O)N[C@@H](C)c8ccc(-c9scnc9C)cc8)C(C)C)on7)C5)CC6)nc4c3F)c12] | [(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl 3-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}oxy)azetidine-1- |
| | | carboxylate |
| 301 | 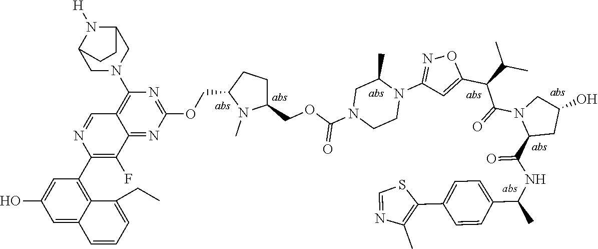 [SMILES: CCc1cccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)[C@H](C)C6)N5C)nc4c3F)c12] | [(2S,5S)-5-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}-1- methylpyrrolidin-2- yl]methyl (3R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- 1,2-oxazol-3-yl}-3- |
| | | methylpiperazine-1- |
| | | carboxylate |
| 302 | 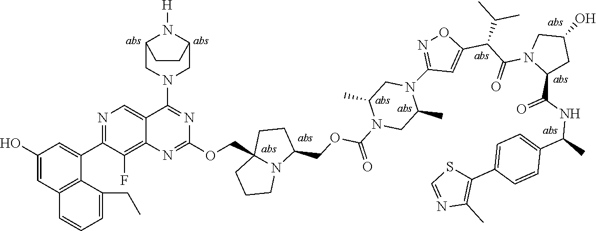 [SMILES: CCc1cccc2cc(O)cc(-c3ncc4c(N5CC6CCC(C5)N6)nc(OC[C@]56CCCN5[C@H](COC(=O)N5C[C@H](C)N(c7cc([C@@H](C(=O)N8C[C@H](O)C[C@H]8C(=O)N[C@@H](C)c8ccc(-c9scnc9C)cc8)C(C)C)on7)C[C@H]5C)CC6)nc4c3F)c12] | [(3S,7aR)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2R,5S)-4-{5- [(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-2,5- |
| | | dimethylpiperazine-1- |
| | | carboxylate |
| 303 | 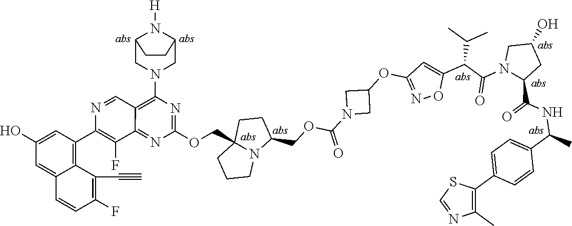 [SMILES: C#Cc1c(F)ccc2cc(O)cc(-c3ncc4c(N5CC6CCC(C5)N6)nc(OC[C@]56CCCN5[C@H](COC(=O)N5CC(Oc7cc([C@@H](C(=O)N8C[C@H](O)C[C@H]8C(=O)N[C@@H](C)c8ccc(-c9scnc9C)cc8)C(C)C)on7)C5)CC6)nc4c3F)c12] | [(3S,7aR)-7a-{[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]methyl}- hexahydro-1H-pyrrolizin- 3-yl]methyl 3-({5-[(2S)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- |
| | | methyl-1-oxobutan-2-yl]- |
| | | 1,2-oxazol-3- |
| | | yl}oxy)azetidine-1- |
| | | carboxylate |
| 304 | 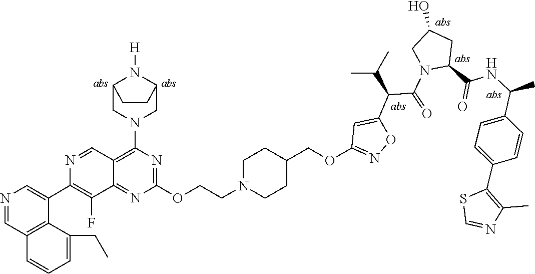 [SMILES: CCc1cccc2cncc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3,8- diazabicyclo[3.2.1]octan- 3-yl}-7-(5- ethylisoquinolin-4-yl)-8- fluoropyrido[4,3- d]pyrimidin-2- yl)oxy]ethyl}piperidin-4- yl)methoxy]-1,2-oxazol- 5-yl}-3-methylbutanoyl]- 4-hydroxy-N-[(1S)-1-[4- (4-methyl-1,3-thiazol-5- yl)phenyl]ethyl] pyrrolidine- 2-carboxamide |
| 305 | 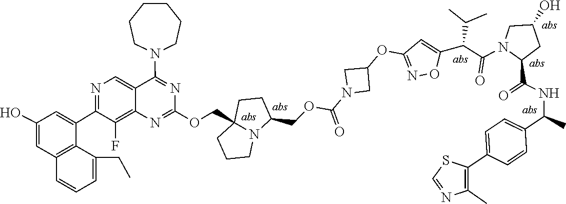 [SMILES: CCc1cccc2cc(O)cc(-c3ncc4c(N5CCCCCC5)nc(OC[C@]56CCCN5[C@H](COC(=O)N5CC(Oc7cc([C@@H](C(=O)N8C[C@H](O)C[C@H]8C(=O)N[C@@H](C)c8ccc(-c9scnc9C)cc8)C(C)C)on7)C5)CC6)nc4c3F)c12] | [(3S,7aR)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methyl 3-({5-[(2S)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}oxy)azetidine-1- |
| | | carboxylate |
| 306 | 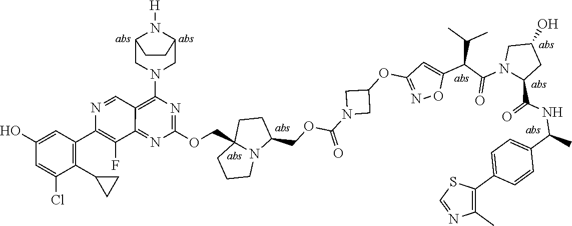 [SMILES: Cc1ncsc1-c1ccc([C@H](C)NC(=O)[C@@H]2C[C@@H](O)CN2C(=O)[C@@H](c2cc(OC3CN(C(=O)OC[C@@H]4CC[C@@]5(COc6nc(N7CC8CCC(C7)N8)c7cnc(-c8cc(O)cc(Cl)c8C8CC8)c(F)c7n6)CCCN45)C3)no2)C(C)C)cc1] | [(3S,7aR)-7a-({[7-(3- chloro-2-cyclopropyl-5- hydroxyphenyl)-4-{3,8- diazabicyclo[3.2.1]octan- 3-yl}-8-fluoropyrido[4,3- d]pyrimidin-2- yl]oxy}methyl)- hexahydro-1H-pyrrolizin- 3-yl]methyl 3-({5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}oxy)azetidine-1- |
| | | carboxylate |
| 307 | 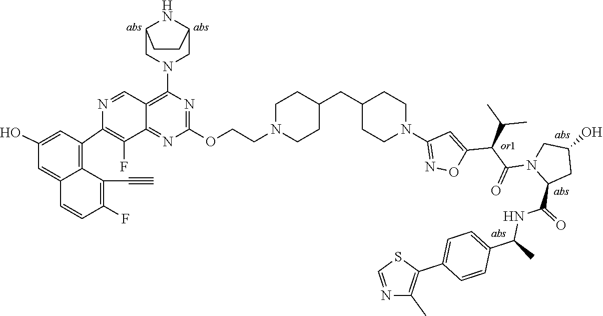 [SMILES: C#Cc1c(F)ccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R*)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 308 | 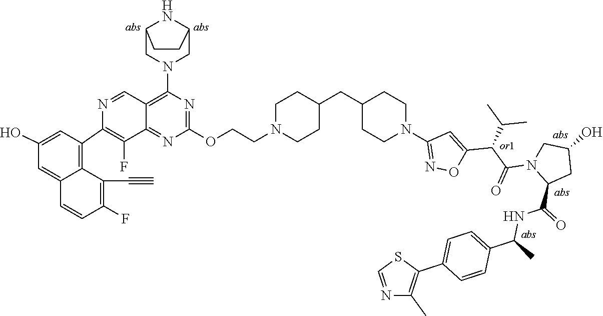 [SMILES: C#Cc1c(F)ccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R*)-2-(3-{4- [(1-{2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 309 | 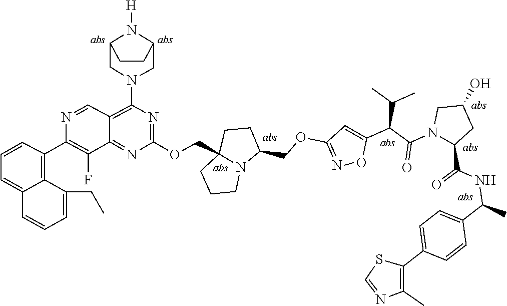 [SMILES: CCc1cccc2cccc(-c3ncc4c(N5CC6CCC(C5)N6)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aR)-7a-{[(4-{3,8- diazabicyclo[3.2.1]octan- 3-yl}-7-(8- ethylnaphthalen-1-yl)-8- fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 310 | 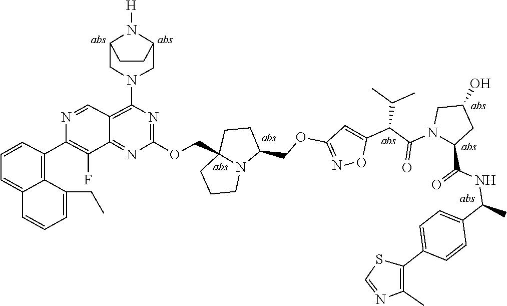 [SMILES: CCc1cccc2cccc(-c3ncc4c(N5CC6CCC(C5)N6)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aR)-7a-{[(4-{3,8- diazabicyclo[3.2.1]octan- 3-yl}-7-(8- ethylnaphthalen-1-yl)-8- fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 311 | 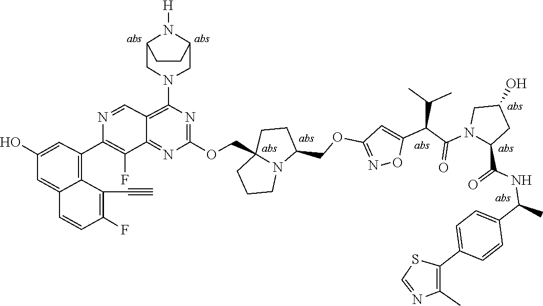 [SMILES: C#Cc1c(F)ccc2cc(O)cc(-c3ncc4c(N5CC6CCC(C5)N6)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aR)-7a-{[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 312 | 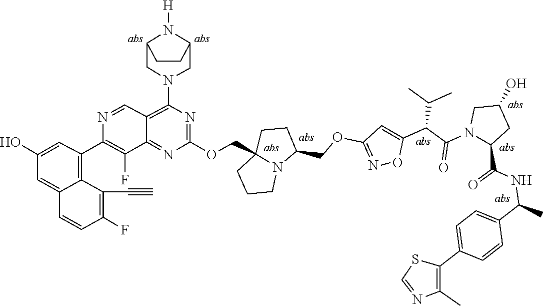 [SMILES: C#Cc1c(F)ccc2cc(O)cc(-c3ncc4c(N5CC6CCC(C5)N6)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aR)-7a-{[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 313 | 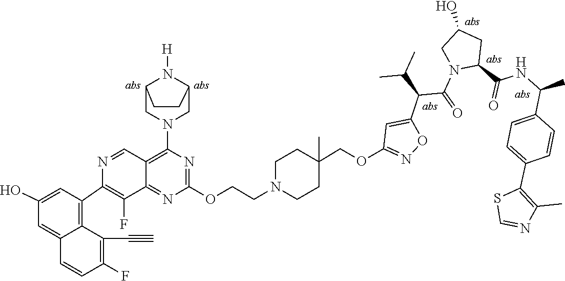 [SMILES: C#Cc1c(F)ccc2cc(O)cc(-c3ncc4c(N5CC6CCC(C5)N6)nc(OCCN5CCC(C)(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]ethyl}-4- methylpiperidin-4- yl)methoxy]-1,2-oxazol- 5-yl}-3-methylbutanoyl]- 4-hydroxy-N-[(1S)-1-[4- (4-methyl-1,3-thiazol-5- yl)phenyl]ethyl] pyrrolidine- 2-carboxamide |
| 314 | 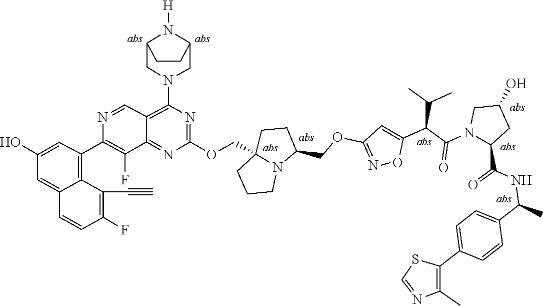 [SMILES: C#Cc1c(F)ccc2cc(O)cc(-c3ncc4c(N5CC6CCC(C5)N6)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aS)-7a-{[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 315 | 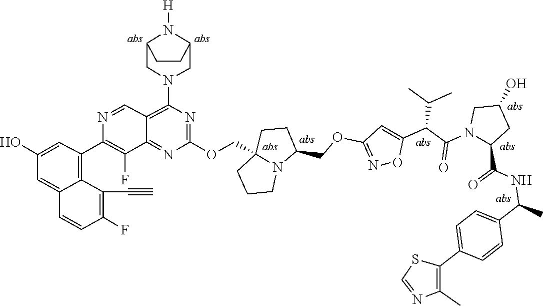 [SMILES: C#Cc1c(F)ccc2cc(O)cc(-c3ncc4c(N5CC6CCC(C5)N6)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aS)-7a-{[(4-{3,8- diazabicyclo[3.2.1]octan- 3-yl}-7-(8-ethynyl-7- fluoro-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 316 | 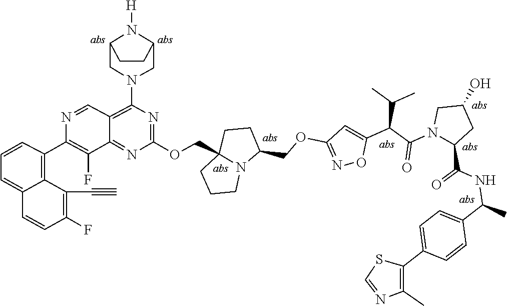 [SMILES: C#Cc1c(F)ccc2cccc(-c3ncc4c(N5CC6CCC(C5)N6)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aR)-7a-{[(4-{3,8- diazabicyclo[3.2.1]octan- 3-yl}-7-(8-ethynyl-7- fluoronaphthalen-1-yl)-8- fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 317 | 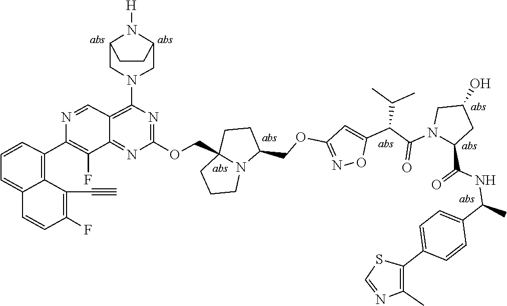 [SMILES: C#Cc1c(F)ccc2cccc(-c3ncc4c(N5CC6CCC(C5)N6)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aR)-7a-{[(4-{3,8- diazabicyclo[3.2.1]octan- 3-yl}-7-(8-ethynyl-7- fluoronaphthalen-1-yl)-8- fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 318 | 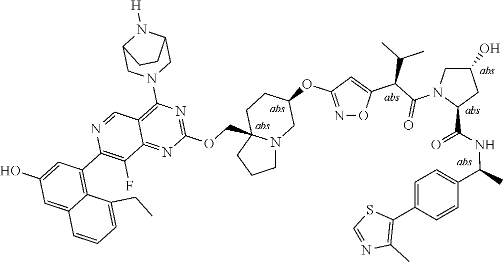 [SMILES: CCc1cccc2cc(O)cc(-c3ncc4c(N5CC6CCC(C5)N6)nc(OC[C@]56CCCN5C[C@H](Oc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(6R,8aR)-8a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- octahydroindolizin-6- yl]oxy}-1,2-oxazol-5-yl)- 3-methylbutanoyl]-4- hydroxy-N-[(1S)-1-[4-(4- methyl-1,3-thiazol-5- yl)phenyl]ethyl] pyrrolidine- 2-carboxamide |
| 319 | 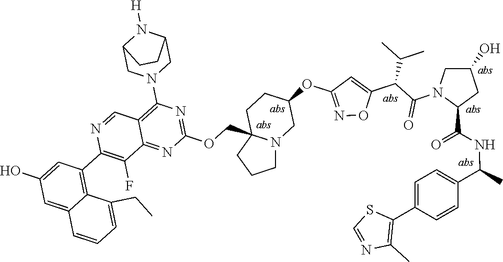 [SMILES: CCc1cccc2cc(O)cc(-c3ncc4c(N5CC6CCC(C5)N6)nc(OC[C@]56CCCN5C[C@H](Oc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(6R,8aR)-8a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- octahydroindolizin-6- yl]oxy}-1,2-oxazol-5-yl)- 3-methylbutanoyl]-4- hydroxy-N-[(1S)-1-[4-(4- methyl-1,3-thiazol-5- yl)phenyl]ethyl] pyrrolidine- 2-carboxamide |
| 320 | 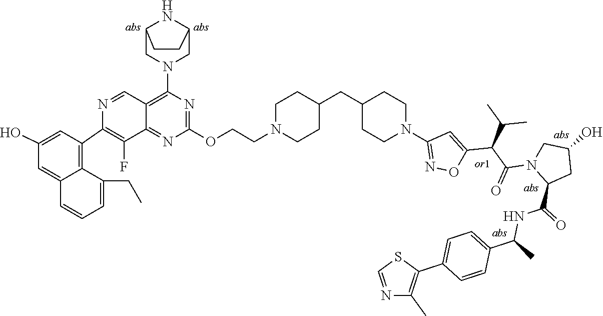 [SMILES: CCc1cccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R*)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 321 | 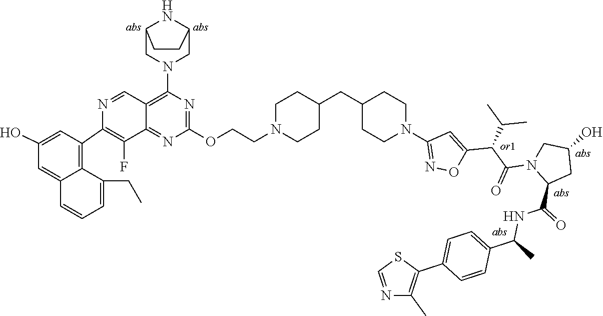 [SMILES: CCc1cccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12] | (2S,4R)-1-[(2R*)-2-(3-{4- [(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methyl]piperidin-1-yl}- 1,2-oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 322 | 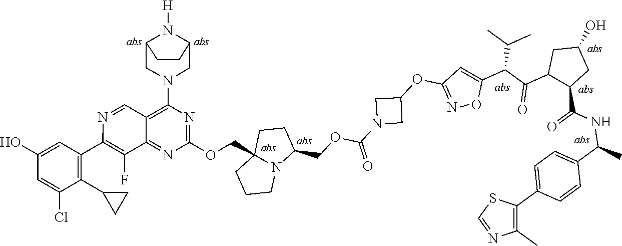 [SMILES: Cc1ncsc1-c1ccc([C@H](C)NC(=O)[C@@H]2C[C@@H](O)CC2C(=O)[C@H](c2cc(OC3CN(C(=O)OC[C@@H]4CC[C@@]5(COc6nc(N7CC8CCC(C7)N8)c7cnc(-c8cc(O)cc(Cl)c8C8CC8)c(F)c7n6)CCCN45)C3)no2)C(C)C)cc1] | [(3S,7aR)-7a-({[7-(3- chloro-2-cyclopropyl-5- hydroxyphenyl)-4-{3,8- diazabicyclo[3.2.1]octan- 3-yl}-8-fluoropyrido[4,3- d]pyrimidin-2- yl]oxy}methyl)- hexahydro-1H-pyrrolizin- 3-yl]methyl 3-({5-[(2S)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- |
| | | yl}oxy)azetidine-1- |
| | | carboxylate |
| 323 | 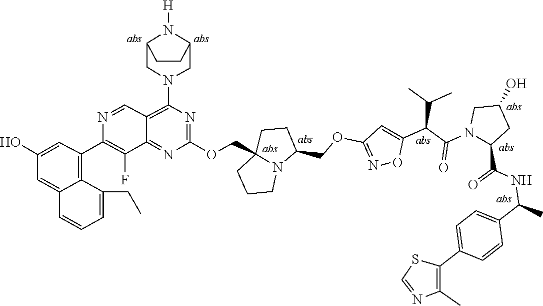 [SMILES: CCc1cccc2cc(O)cc(-c3ncc4c(N5CC6CCC(C5)N6)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aR)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 324 | 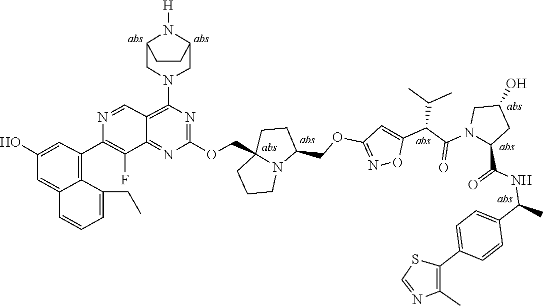 [SMILES: CCc1cccc2cc(O)cc(-c3ncc4c(N5CC6CCC(C5)N6)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aR)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 325 | 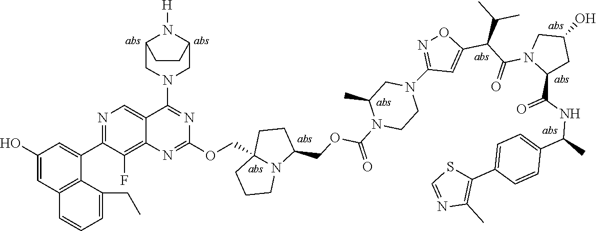 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)C[C@@H]5C)CC6)nc4c3F)c12] | [(3S,7aS)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2S)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-2- |
| | | methylpiperazine-1- |
| | | carboxylate |
| 326 | 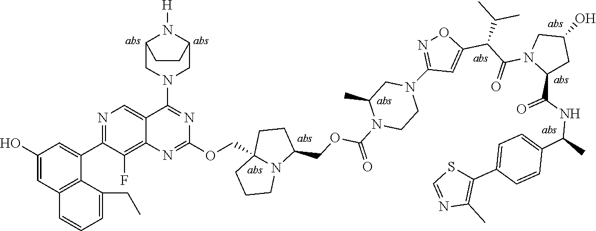 [SMILES: CCc1cccc2cc(O)cc(-c3ncc4c(N5CC6CCC(C5)N6)nc(OC[C@@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)C[C@@H]5C)CC6)nc4c3F)c12] | [(3S,7aS)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (2S)-4-{5- [(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-2- |
| | | methylpiperazine-1- |
| | | carboxylate |
| 327 | 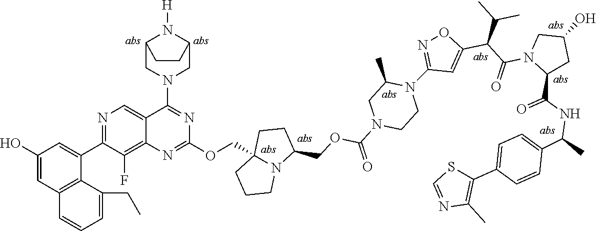 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)[C@H](C)C5)CC6)nc4c3F)c12] | [(3S,7aS)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (3R)-4-{5- [(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-3- |
| | | methylpiperazine-1- |
| | | carboxylate |
| 328 | 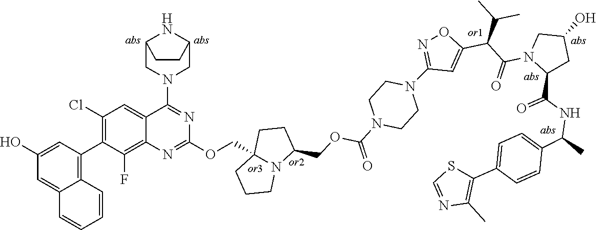 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@]5(COc6nc(N7CC8CCC(C7)N8)c7cc(Cl)c(-c8cc(O)cc9ccccc89)c(F)c7n6)CCCN45)CC3)no2)C(C)C)cc1] | [(3R*,7aR**)-7a-{[(6- chloro-4-{3,8- diazabicyclo[3.2.1]octan- 3-yl}-8-fluoro-7-(3- hydroxynaphthalen-1- yl)quinazolin-2- yl)oxy]methyl}- hexahydro-1H-pyrrolizin- 3-yl]methyl 4-{5-[(2R***)- 1-[(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 329 | 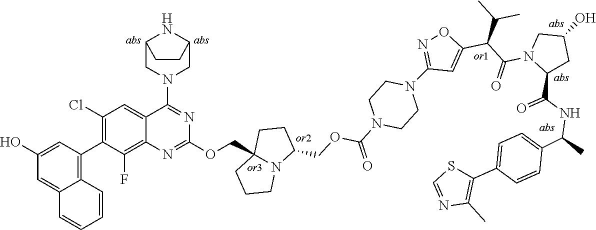 [SMILES: Cc1ncsc1-c1ccc([C@H](C)NC(=O)[C@@H]2C[C@@H](O)CN2C(=O)[C@@H](c2cc(N3CCN(C(=O)OC[C@H]4CC[C@@]5(COc6nc(N7CC8CCC(C7)N8)c7cc(Cl)c(-c8cc(O)cc9ccccc89)c(F)c7n6)CCCN45)CC3)no2)C(C)C)cc1] | [(3R*,7aR**)-7a-{[(6- chloro-4-{3,8- diazabicyclo[3.2.1]octan- 3-yl}-8-fluoro-7-(3- hydroxynaphthalen-1- yl)quinazolin-2- yl)oxy]methyl}- hexahydro-1H-pyrrolizin- 3-yl]methyl 4-{5-[(2R***)- 1-[(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 330 | 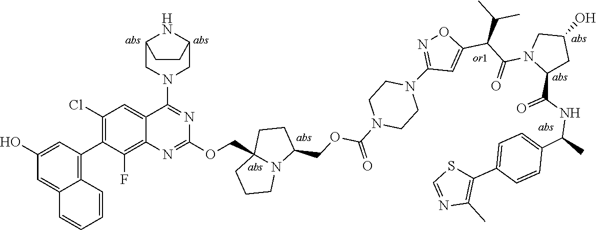 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cc(Cl)c(-c8cc(O)cc9ccccc89)c(F)c7n6)CCCN45)CC3)no2)C(C)C)cc1] | [(3S,7aR)-7a-{[(6-chloro- 4-{3,8- diazabicyclo[3.2.1]octan- 3-yl}-8-fluoro-7-(3- hydroxynaphthalen-1- yl)quinazolin-2- yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 331 | 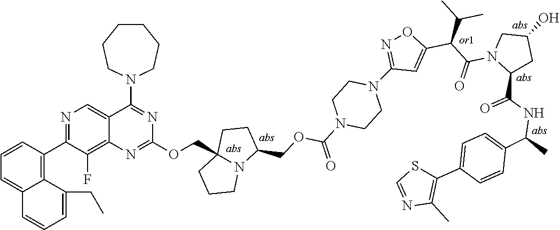 [SMILES: CCc1cccc2cccc(-c3ncc4c(N5CCCCCC5)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3S,7aR)-7a-({[4- (azepan-1-yl)-7-(8- ethylnaphthalen-1-yl)-8- fluoropyrido[4,3- d]pyrimidin-2- yl]oxy}methyl)- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- |
| | | yl}piperazine-1- |
| | | carboxylate |
| 332 | 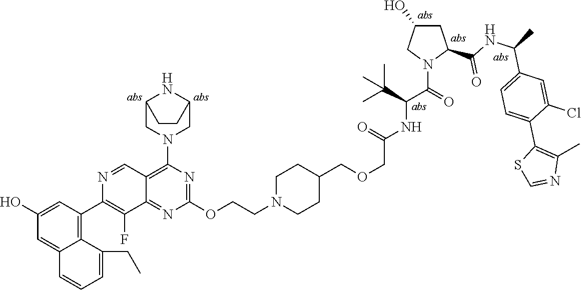 [SMILES: CCc1cccc2cc(O)cc(-c3ncc4c(N5CC6CCC(C5)N6)nc(OCCN5CCC(COCC(=O)N[C@H](C(=O)N6C[C@H](O)C[C@H]6C(=O)N[C@@H](C)c6ccc(-c7scnc7C)c(Cl)c6)C(C)(C)C)CC5)nc4c3F)c12] | (2S,4R)-N-[(1S)-1-[3- chloro-4-(4-methyl-1,3- thiazol-5- yl)phenyl]ethyl]-1-[(2S)- 2-{2-[(1-{2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}piperidin-4- yl)methoxy]acetamido}- 3,3-dimethylbutanoyl]-4- hydroxypyrrolidine-2- carboxamide |
| 333 | 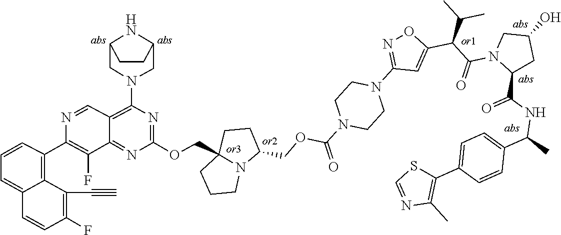 [SMILES: C#Cc1c(F)ccc2cccc(-c3ncc4c(N5CC6CCC(C5)N6)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R*,7aR**)-7a-{[(4- {3,8- diazabicyclo[3.2.1]octan- 3-yl}-7-(8-ethynyl-7- fluoronaphthalen-1-yl)-8- fluoropyrido[4,3- d]pyrimidin-2- yl)oxy]methyl}- hexahydro-1H-pyrrolizin- 3-yl]methyl 4-{5-[(2R***)- 1-[(2S,4R)-4-hydroxy-2- {[(1S)-1-[4-(4-methyl- 1,3-thiazol-5- yl)phenyl]ethyl]carbamoyl} pyrrolidin-1-yl]-3- |
| | | methyl-1-oxobutan-2-yl]- |
| | | 1,2-oxazol-3- |
| | | yl}piperazine-1- |
| | | carboxylate |
| 334 | 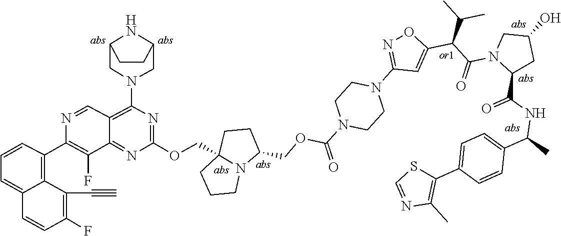 [SMILES: C#Cc1c(F)ccc2cccc(-c3ncc4c(N5CC6CCC(C5)N6)nc(OC[C@@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | [(3R,7aS)-7a-{[(4-{3,8- diazabicyclo[3.2.1]octan- 3-yl}-7-(8-ethynyl-7- fluoronaphthalen-1-yl)-8- fluoropyrido[4,3- d]pyrimidin-2- yl)oxy]methyl}- hexahydro-1H-pyrrolizin- 3-yl]methyl 4-{5-[(2R*)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- |
| | | 1,2-oxazol-3- |
| | | yl}piperazine-1- |
| | | carboxylate |
| 335 | 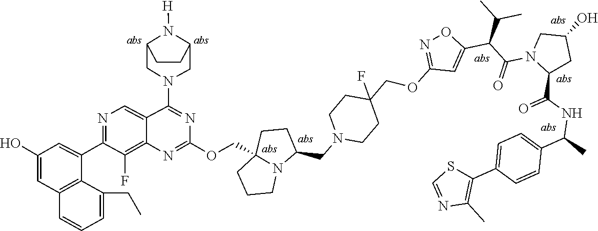 [SMILES: CCc1cccc2cc(O)cc(-c3ncc4c(N5CC6CCC(C5)N6)nc(OC[C@@]56CCCN5[C@H](CN5CCC(F)(COc7cc([C@H](C(=O)N8C[C@H](O)C[C@H]8C(=O)N[C@@H](C)c8ccc(-c9scnc9C)cc8)C(C)C)on7)CC5)CC6)nc4c3F)c12] | (2S,4R)-1-[(2R)-2-{3-[(1- {[(3S,7aS)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl}-4- fluoropiperidin-4- yl)methoxy]-1,2-oxazol- 5-yl}-3-methylbutanoyl]- 4-hydroxy-N-[(1S)-1-[4- (4-methyl-1,3-thiazol-5- |
| | | yl)phenyl]ethyl] |
| | | pyrrolidine- |
| | | 2-carboxamide |
| 336 | 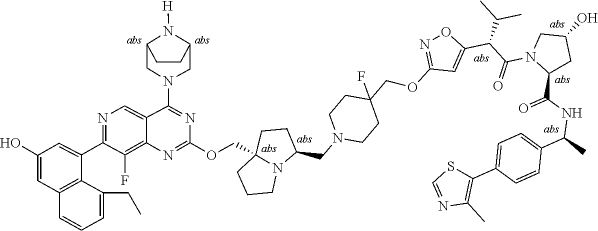 [SMILES: CCc1cccc2cc(O)cc(-c3ncc4c(N5CC6CCC(C5)N6)nc(OC[C@@]56CCCN5[C@H](CN5CCC(F)(COc7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12] | (2S,4R)-1-[(2S)-2-{3-[(1- {[(3S,7aS)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl}-4- fluoropiperidin-4- yl)methoxy]-1,2-oxazol- 5-yl}-3-methylbutanoyl]- 4-hydroxy-N-[(1S)-1-[4- (4-methyl-1,3-thiazol-5- |
| | | yl)phenyl]ethyl] |
| | | pyrrolidine- |
| | | 2-carboxamide |
| 337 | 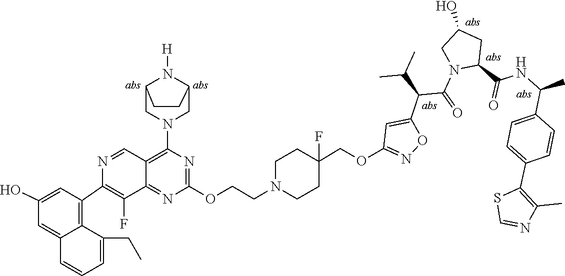 [SMILES: CCc1cccc2cc(O)cc(-c3ncc4c(N5CC6CCC(C5)N6)nc(OCCN5CCC(F)(COc6cc([C@H](C(=O)N7C[C@H](O)C[C@H]7C(=O)N[C@@H](C)c7ccc(-c8scnc8C)cc7)C(C)C)on6)CC5)nc4c3F)c12] | (2S,4R)-1-[(2R)-2-{3-[(1- {2-[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]ethyl}-4- fluoropiperidin-4- yl)methoxy]-1,2-oxazol- 5-yl}-3-methylbutanoyl]- 4-hydroxy-N-[(1S)-1-[4- (4-methyl-1,3-thiazol-5- yl)phenyl]ethyl] pyrrolidine- 2-carboxamide |
| 338 | 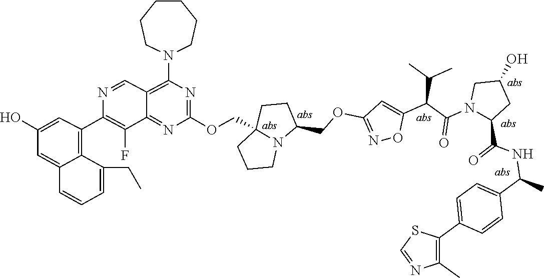 [SMILES: CCc1cccc2cc(O)cc(-c3ncc4c(N5CCCCCC5)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2R)-2-(3- {[(3S,7aS)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 339 | 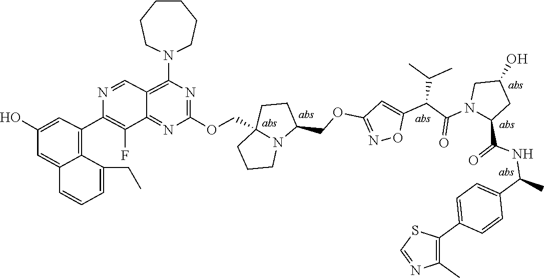 [SMILES: CCc1cccc2cc(O)cc(-c3ncc4c(N5CCCCCC5)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12] | (2S,4R)-1-[(2S)-2-(3- {[(3S,7aS)-7a-({[4- (azepan-1-yl)-7-(8-ethyl- 3-hydroxynaphthalen-1- yl)-8-fluoropyrido[4,3- d]pyrimidin-2- yl]oxy}methyl)- hexahydro-1H-pyrrolizin- 3-yl]methoxy}-1,2- oxazol-5-yl)-3- methylbutanoyl]-4- hydroxy-N-[(1S)-1-[4-(4- methyl-1,3-thiazol-5- yl)phenyl]ethyl] pyrrolidine- 2-carboxamide |
| 340 | 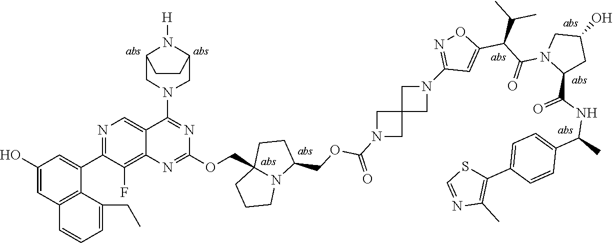 [SMILES: CCc1cccc2cc(O)cc(-c3ncc4c(N5CC6CCC(C5)N6)nc(OC[C@]56CCCN5[C@H](COC(=O)N5CC7(C5)CN(c5cc([C@H](C(=O)N8C[C@H](O)C[C@H]8C(=O)N[C@@H](C)c8ccc(-c9scnc9C)cc8)C(C)C)on5)C7)CC6)nc4c3F)c12] | [(3S,7aR)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl 6-{5-[(2R)-1- [(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3-yl}-2,6- |
| | | diazaspiro[3.3]heptane- |
| | | 2-carboxylate |
| 341 | 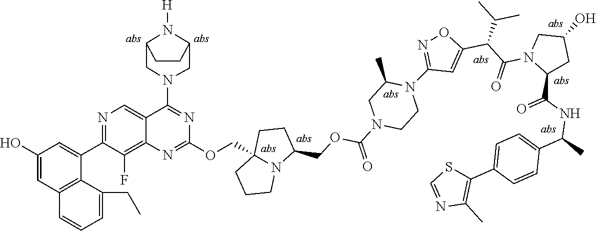 [SMILES: CCc1cccc2cc(O)cc(-c3ncc4c(N5CC6CCC(C5)N6)nc(OC[C@@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)[C@H](C)C5)CC6)nc4c3F)c12] | [(3S,7aS)-7a-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 3-yl]methyl (3R)-4-{5- [(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3- methyl-1-oxobutan-2-yl]- |
| | | 1,2-oxazol-3-yl}-3- |
| | | methylpiperazine-1- |
| | | carboxylate |
| 342 | 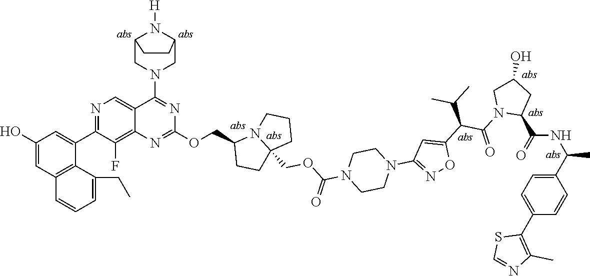 [SMILES: CCc1cccc2cc(O)cc(-c3ncc4c(N5CC6CCC(C5)N6)nc(OC[C@@H]5CC[C@]6(COC(=O)N7CCN(c8cc([C@H](C(=O)N9C[C@H](O)C[C@H]9C(=O)N[C@@H](C)c9ccc(-c%10scnc%10C)cc9)C(C)C)on8)CC7)CCCN56)nc4c3F)c12] | [(3S,7aS)-3-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 7a-yl]methyl 4-{5-[(2R)- 1-[(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- yl}piperazine-1- carboxylate |
| 343 | 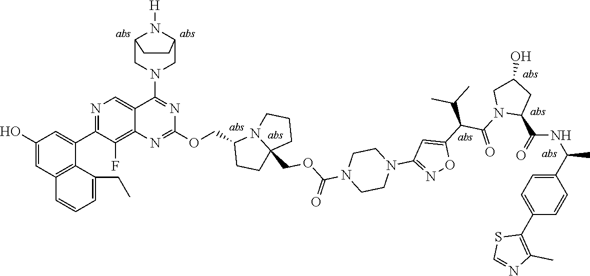 [SMILES: CCc1cccc2cc(O)cc(-c3ncc4c(N5CC6CCC(C5)N6)nc(OC[C@H]5CC[C@@]6(COC(=O)N7CCN(c8cc([C@H](C(=O)N9C[C@H](O)C[C@H]9C(=O)N[C@@H](C)c9ccc(-c%10scnc%10C)cc9)C(C)C)on8)CC7)CCCN56)nc4c3F)c12] | [(3R,7aR)-3-{[(4-{3,8- diazabicyclo[3.2.1]octan- 3-yl}-7-(8-ethyl-3- hydroxynaphthalen-1-yl)- 8-fluoropyrido[4,3- d]pyrimidin-2- yl)oxy]methyl}- hexahydro-1H-pyrrolizin- 7a-yl]methyl 4-{5-[(2R)- 1-[(2S,4R)-4-hydroxy-2- {[(1S)-1-[4-(4-methyl- 1,3-thiazol-5- yl)phenyl]ethyl]carbamoyl} pyrrolidin-1-yl]-3- methyl-1-oxobutan-2-yl]- 1,2-oxazol-3- yl}piperazine-1- carboxylate |

A compound of the disclosure may be synthesized using standard synthetic methods and procedures for the preparation of organic molecules and functional group transformations and manipulations, including the use of protective groups, as can be obtained from the relevant scientific literature or from standard reference textbooks in the field in view of this disclosure. Although not limited to any one or several sources, recognized reference textbooks of organic synthesis include: Smith, M. B.; March, J. March's Advanced Organic Chemistry: Reactions, Mechanisms, and Structure, 5th ed.; John Wiley & Sons: New York, 2001; and Greene, T. W.; Wuts, P. G. M. Protective Groups in Organic Synthesis, 3^{rd}; John Wiley & Sons: New York, 1999. The synthetic methods described in International Publication No. WO/2021/207172 are incorporated herein by reference in their entireties.

### Methods of Ubiquitinating/Degrading a Target Protein in a Cell

The present disclosure provides a method of ubiquitinating/degrading a target protein in a cell. The method comprises administering a bifunctional composition comprising an E3 ubiquitin ligase binding moiety and a protein targeting moiety, preferably linked through a linker moiety, as otherwise described herein, wherein the E3 ubiquitin ligase binding moiety is coupled to the protein targeting moiety and wherein the E3 ubiquitin ligase binding moiety recognizes a ubiquitin pathway protein (e.g., a ubiquitin ligase, preferably an E3 ubiquitin ligase) and the protein targeting moiety recognizes the target protein such that the target protein will be ubiquitinated when the target protein is placed in proximity to the ubiquitin ligase, resulting in degradation/inhibition of the effects of the target protein and the control of protein levels. The control of protein levels afforded by the present disclosure provides treatment of a disease state or condition, which is modulated through the target protein by lowering the level of that protein in the cells of a patient

In some embodiments, a bifunctional compound described herein binds to KRAS. In some embodiments, a bifunctional compound described herein reversibly binds to KRAS. In some embodiments, the KTM of a bifunctional compound binds to KRAS. In some embodiments, the KTM of a bifunctional compound reversibly binds KRAS.

In some embodiments, a bifunctional compound described herein binds to, and causes the degradation of KRAS. In some embodiments, a bifunctional compound described herein reversibly binds to, and causes the degradation of KRAS.

In aspects, disclosed herein are compounds of Formula (I), or a pharmaceutically acceptable salt, enantiomer, stereoisomer, solvate, polymorph, isotopic derivative, or prodrug thereof, that degrades KRAS.

In some embodiments, KRAS exists in two isoforms: KRAS4A (also known as KRAS2A) and KRAS4B (also known as KRAS2B). In some embodiments, these isoforms differ in the HVR residues 167-189. In some embodiments, KRAS residues 151, 153, 165 and 166 are dissimilar between isoforms KRAS4A and KRAS4B.

KRAS comprises a flexible, membrane anchoring, C-terminal structural element, named the hypervariable region (HVR). Because KRAS signaling occurs at the membrane, the HVR undergoes a post-translational modification including farnesylation at C185, proteolytic cleavage of the three terminal residues, and methylation of the terminal carboxyl group of C185. A polybasic region of the HVR, composed of multiple lysine residues, is also involved in the membrane association. As KRAS4A does not contain this polybasic region, it is further palmitoylated at an additional cysteine residue C180.

In some embodiments, the KRAS is isoform KRAS4B. In some embodiments, the KRAS4B isoform comprises the amino acid sequence of SEQ ID NO: 1.

SEQ ID NO: 1
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM

In some embodiments, the KRAS is isoform KRAS4A. In some embodiments, the KRAS4A isoform comprises the amino acid sequence of SEQ ID NO: 3.

SEQ ID NO: 3
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

In some embodiments, the KRAS is a mutant KRAS. In some embodiments, the mutant KRAS is selected from one or more of KRAS G12D, KRAS G12C, KRAS G12V, KRAS G12S, KRAS G12R, KRAS G12A, and KRAS G13C. In some embodiments, the mutant KRAS is selected from one or more of KRAS G12D, KRAS G12C, and KRAS G12V. In some embodiments, the mutant KRAS is a G12D mutant. In some embodiments, the mutant KRAS is a G12C mutant. In some embodiments, the mutant KRAS is a G12V mutant. In some embodiments, the mutant KRAS G12D comprises the amino acid sequence of SEQ ID NO: 2. In some embodiments, the mutant KRAS G12D comprises the amino acid sequence of SEQ ID NO: 4.

In some embodiments, the KRAS is a mammalian KRAS. In some embodiments, the KRAS is a human KRAS. In some embodiments, the KRAS is a non-human primate KRAS. In some embodiments, a bifunctional compound described herein binds to KRAS comprising the amino acid sequence of SEQ ID NO: 1. In some embodiments, a bifunctional compound described herein binds to KRAS comprising the amino acid sequence of SEQ ID NO: 2. In some embodiments, a bifunctional compound described herein binds to KRAS comprising the amino acid sequence of SEQ ID NO: 3. In some embodiments, a bifunctional compound described herein binds to KRAS comprising the amino acid sequence of SEQ ID NO: 4. In some embodiments, a bifunctional compound described herein binds to, and causes the degradation of KRAS comprising the amino acid sequence of SEQ ID NO: 1. In some embodiments, a bifunctional compound described herein binds to, and causes the degradation of KRAS comprising the amino acid sequence of SEQ ID NO: 2. In some embodiments, a bifunctional compound described herein binds to, and causes the degradation of KRAS comprising the amino acid sequence of SEQ ID NO: 3. In some embodiments, a bifunctional compound described herein binds to, and causes the degradation of KRAS comprising the amino acid sequence of SEQ ID NO: 4.

In some embodiments, a bifunctional compound described herein binds to a KRAS mutant comprising an amino acid sequence with at least 80%, at least 85%, at least 90%, at least 95%, or at least 99% identity to SEQ ID NO: 1. In some embodiments, a bifunctional compound described herein binds to, and causes the degradation of a KRAS mutant comprising an amino acid sequence with at least 80%, at least 85%, at least 90%, at least 95%, or at least 99% identity to SEQ ID NO: 1.

In some embodiments, a bifunctional compound described herein binds to a KRAS G12D mutant comprising an amino acid sequence with at least 80%, at least 85%, at least 90%, at least 95%, or at least 99% identity to SEQ ID NO: 2. In some embodiments, a bifunctional compound described herein binds to, and causes the degradation of a KRAS G12D mutant comprising an amino acid sequence with at least 80%, at least 85%, at least 90%, at least 95%, or at least 99% identity to SEQ ID NO: 2.

In some embodiments, a bifunctional compound described herein binds to a KRAS mutant comprising an amino acid sequence with at least 80%, at least 85%, at least 90%, at least 95%, or at least 99% identity to SEQ ID NO: 3. In some embodiments, a bifunctional compound described herein binds to, and causes the degradation of a KRAS mutant comprising an amino acid sequence with at least 80%, at least 85%, at least 90%, at least 95%, or at least 99% identity to SEQ ID NO: 3.

In some embodiments, a bifunctional compound described herein binds to a KRAS G12D mutant comprising an amino acid sequence with at least 80%, at least 85%, at least 90%, at least 95%, or at least 99% identity to SEQ ID NO: 4. In some embodiments, a bifunctional compound described herein binds to, and causes the degradation of a KRAS G12D mutant comprising an amino acid sequence with at least 80%, at least 85%, at least 90%, at least 95%, or at least 99% identity to SEQ ID NO: 4.

SEQ ID NO: 2
MTEYKLVVVGADGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM
SEQ ID NO: 4
MTEYKLVVVGADGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM

In some embodiments, the bifunctional compound described herein binds to all KRAS mutants and isoforms. In some embodiments, the bifunctional compound described herein binds to, and causes the degradation of all KRAS mutants and isoforms.

In some embodiments, the present disclosure is directed to a method of treating a patient in need for a disease state or condition modulated through a protein where the degradation of that protein will produce a therapeutic effect in that patient, the method comprising administering to a patient in need an effective amount of a compound of Formula (I), optionally in combination with another anti-cancer agent. The disease state or condition may be a disease caused by a microbial agent or other exogenous agent such as a virus, bacteria, fungus, protozoa, or other microbe or may be a disease state caused by overexpression of a protein, which leads to a disease state and/or condition.

### Methods of Treatment

In aspects, disclosed herein are methods of treating and/or preventing a disease or disorder in a subject, the method comprising administering to the subject in need thereof a therapeutically effective amount of a bifunctional compound of the disclosure, or a pharmaceutically acceptable salt, enantiomer, stereoisomer, solvate, or isotopic derivative thereof.

In some embodiments, the disease or disorder is causally related to KRAS. In some embodiments, the disease or disorder is related to KRAS activity, overactivity, constitutive activity, expression, overexpression, or accumulation.

In some embodiments, the disease or disorder is cancer. In some embodiments, the cancer is pancreatic cancer, colon cancer, colorectal cancer, lung cancer, non-small cell lung cancer, endometrial cancer, cervical cancer, bladder cancer, liver cancer, myeloid leukemia, ovarian cancer or breast cancer.

In aspects, disclosed herein are methods of treating and/or preventing cancer comprising administering to a subject in need thereof a therapeutically effective amount of a compound of the disclosure, or a pharmaceutically acceptable salt, enantiomer, stereoisomer, solvate, polymorph, isotopic derivative, or prodrug thereof, in combination with one or more additional anti-cancer agents. In some embodiments, the disease or disorder is cancer. In some embodiments, the cancer is pancreatic cancer, colon cancer, colorectal cancer, lung cancer, non-small cell lung cancer, endometrial cancer, cervical cancer, bladder cancer, liver cancer, myeloid leukemia, ovarian cancer or breast cancer.

The methods of treating cancer described herein may result in a reduction in tumor size. Alternatively, or in addition, the cancer is metastatic cancer and this method of treatment includes inhibition of metastatic cancer cell invasion.

In one aspect, treating cancer results in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by 5% or greater relative to its volume prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In another aspect, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means.

In a preferred aspect, an increase in average survival time of a population may be measured, for example, by calculating for a population the average length of survival following initiation of treatment with an active agent or compound of the disclosure. In another preferred aspect, an increase in average survival time of a population may also be measured, for example, by calculating for a population the average length of survival following completion of a first round of treatment with an active agent or compound of the disclosure.

In another aspect, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured by calculating for a population the average length of survival following initiation of treatment with an active agent or compound of the disclosure. In another preferred aspect, an increase in average survival time of a population may be measured by calculating for a population the average length of survival following completion of a first round of treatment with a compound of the disclosure.

In another aspect, treating cancer results in a decrease in tumor growth rate. Preferably, after treatment, tumor growth rate is reduced by at least 5% relative to growth rate prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured by measuring an increase in the diameter of a tumor after a prior tumor shrinkage that followed treatment. In another preferred aspect, a decrease in tumor regrowth is indicated by failure of tumors to reoccur after treatment has stopped.

The dosages of the compound of the disclosure for any of the methods and uses described herein vary depending on the agent, the age, weight, and clinical condition of the recipient subject, and the experience and judgment of the clinician or practitioner administering the therapy, among other factors affecting the selected dosage.

In a non-limiting embodiment, the therapeutically effective amount of the compound of the disclosure may be administered one or more times over a day for up to 30 or more days, followed by 1 or more days of non-administration of the compound. This type of treatment schedule, i.e., administration of a the compound of the disclosure on consecutive days followed by non-administration of the compound on consecutive days may be referred to as a treatment cycle. A treatment cycle may be repeated as many times as necessary to achieve the intended affect.

In some embodiments, the therapeutically effective amount of the compound of the disclosure is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, twenty, twenty-five, thirty consecutive days, or, once, twice, three times, four times, or more daily, in single or divided doses, for 2 months, 3 months, 4 months, 5 months, 6 months, or longer.

In some embodiments, the therapeutically effective amount of the compound of the disclosure is about 10 to about 40 mg, about 20 to about 50 mg, about 30 to about 60 mg, about 40 to about 70 mg, about 50 to about 80 mg, about 60 to about 90 mg, about 70 to about 100 mg, about 80 to about 110 mg, about 90 to about 120 mg, about 100 to about 130 mg, about 110 to about 140 mg, about 120 to about 150 mg, about 130 to about 160 mg, about 140 to about 170 mg, about 150 to about 180 mg, about 160 to about 190 mg, about 170 to about 200 mg, about 180 to about 210 mg, about 190 to about 220 mg, about 200 to about 230 mg, about 210 to about 240 mg, about 220 to about 250 mg, about 230 to about 260 mg, about 240 to about 270 mg, about 250 to about 280 mg, about 260 to about 290 mg, about 270 to about 300 mg, about 280 to about 310 mg, about 290 to about 320 mg, about 300 to about 330 mg, about 310 to about 340 mg, about 320 to about 350 mg, about 330 to about 360 mg, about 340 to about 370 mg, about 350 to about 380 mg, about 360 to about 390 mg, about 370 to about 400 mg, about 380 to about 410 mg, about 390 to about 420 mg, about 400 to about 430 mg, about 410 to about 440 mg, about 420 to about 450 mg, about 430 to about 460 mg, about 440 to about 470 mg, about 450 to about 480 mg, about 460 to about 490 mg, about 470 to about 500 mg, about 480 to about 510 mg, about 490 to about 520 mg, about 500 to about 530 mg, about 510 to about 540 mg, about 520 to about 550 mg, about 530 to about 560 mg, about 540 to about 570 mg, about 550 to about 580 mg, about 560 to about 590 mg, about 570 to about 600 mg, about 580 to about 610 mg, about 590 to about 620 mg, about 600 to about 630 mg, about 610 to about 640 mg, about 620 to about 650 mg, about 630 to about 660 mg, about 640 to about 670 mg, about 650 to about 680 mg, about 660 to about 690 mg, about 670 to about 700 mg, about 680 to about 710 mg, about 690 to about 720 mg, about 700 to about 730 mg, about 710 to about 740 mg, about 720 to about 750 mg, about 730 to about 760 mg, about 740 to about 770 mg, about 750 to about 780 mg, about 760 to about 790 mg, about 770 to about 800 mg, about 780 to about 810 mg, about 790 to about 820 mg, about 800 to about 830 mg, about 810 to about 840 mg, about 820 to about 850 mg, about 830 to about 860 mg, about 840 to about 870 mg, about 850 to about 880 mg, about 860 to about 890 mg, about 870 to about 900 mg, about 880 to about 910 mg, about 890 to about 920 mg, about 900 to about 930 mg, about 910 to about 940 mg, about 920 to about 950 mg, about 930 to about 960 mg, about 940 to about 970 mg, about 950 to about 980 mg, about 960 to about 990 mg, or about 970 to about 1,000 mg administered once, twice, three times, four times, or more daily in single or divided doses (which dose may be adjusted for the patient's weight in kg, body surface area in m², and/or age in years).

In some embodiments, the therapeutically effective amount of the compound of the disclosure is about 70 mg to about 1000 mg administered once, twice, three times, four times, or more daily in single or divided doses (which dose may be adjusted for the patient's weight in kg, body surface area in m², and/or age in years).

In some embodiments, the therapeutically effective amount of the compound of the disclosure is about 70 mg, 105 mg, 140 mg, 175 mg, 210 mg, 245 mg, 280 mg, 315 mg, 350 mg, 385 mg, 420 mg, 455 mg, 490 mg, 525 mg, 560 mg, 595 mg, 630 mg, 665 mg, or 700 mg administered once, twice, three times, four times, or more daily in single or divided doses (which dose may be adjusted for the patient's weight in kg, body surface area in m², and/or age in years).

The therapeutically effective amount of the compound of the disclosure can also range from about 0.01 mg/kg per day to about 100 mg/kg per day. In an aspect, therapeutically effective amount of the compound of the disclosure can range from about 0.05 mg/kg per day to about 10 mg/kg per day. In an aspect, therapeutically effective amount of the compound of the disclosure can range from about 0.075 mg/kg per day to about 5 mg/kg per day. In an aspect, therapeutically effective amount of the compound of the disclosure can range from about 0.10 mg/kg per day to about 1 mg/kg per day. In an aspect, therapeutically effective amount of the compound of the disclosure can range from about 0.20 mg/kg per day to about 0.70 mg/kg per day.

In some embodiments, the therapeutically effective amount of the compound of the disclosure is about 0.10 mg/kg per day, about 0.15 mg/kg per day, about 0.20 mg/kg per day, about 0.25 mg/kg per day, about 0.30 mg/kg per day, about 0.35 mg/kg per day, about 0.40 mg/kg per day, about 0.45 mg/kg per day, about 0.50 mg/kg per day, about 0.55 mg/kg per day, about 0.60 mg/kg per day, about 0.65 mg/kg per day, about 0.70 mg/kg per day, about 0.75 mg/kg per day, about 0.80 mg/kg per day, about 0.85 mg/kg per day, about 0.90 mg/kg per day, about 0.95 mg/kg per day, or about 1.00 mg/kg per day.

In some embodiments, the therapeutically effective amount of the compound of the disclosure is about 1.05 mg/kg per day, about 1.10 mg/kg per day, about 1.15 mg/kg per day, about 1.20 mg/kg per day, about 1.25 mg/kg per day, about 1.30 mg/kg per day, about 1.35 mg/kg per day, about 1.40 mg/kg per day, about 1.45 mg/kg per day, about 1.50 mg/kg per day, about 1.55 mg/kg per day, about 1.60 mg/kg per day, about 1.65 mg/kg per day, about 1.70 mg/kg per day, about 1.75 mg/kg per day, about 1.80 mg/kg per day, about 1.85 mg/kg per day, about 1.90 mg/kg per day, about 1.95 mg/kg per day, or about 2.00 mg/kg per day.

In some embodiments, the therapeutically effective amount of the compound of the disclosure is about 2 mg/kg per day, about 2.5 mg/kg per day, about 3 mg/kg per day, about 3.5 mg/kg per day, about 4 mg/kg per day, about 4.5 mg/kg per day, about 5 mg/kg per day, about 5.5 mg/kg per day, about 6 mg/kg per day, about 6.5 mg/kg per day, about 7 mg/kg per day, about 7.5 mg/kg per day, about 8.0 mg/kg per day, about 8.5 mg/kg per day, about 9.0 mg/kg per day, about 9.5 mg/kg per day, or about 10 mg/kg per day.

In some embodiments, the therapeutically effective amount of the compound of the disclosure is administered to the subject once daily. In some embodiments, this daily dose of a compound of the compound of the disclosure may administered to the subject all at once. In some embodiments, this daily dose of the compound of the disclosure may administered to the subject in two portions (i.e., a divided dose). In some embodiments, this daily dose of the compound of the disclosure may administered to the subject in three divided doses. In some embodiments, this daily dose of the compound of the disclosure may administered to the subject in four divided doses. In some embodiments, this daily dose of the compound of the disclosure may be administered to the subject in five or more divided doses. In some embodiments, these portions or divided doses are administered to the subject at regular intervals throughout the day, for example, every 12 hours, every 8 hours, every 6 hours, every 5 hours, every 4 hours, etc.

The therapeutically effective amount of the compound of the disclosure can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the compound of the disclosure or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, once every two weeks, or monthly depending on half-life and clearance rate of the particular formulation.

### Pharmaceutical Compositions

In aspects, this application pertains to a pharmaceutical composition comprising a bifunctional compound as disclosed herein and one or more pharmaceutically acceptable excipients.

In some embodiments, the compound of the disclosure is formulated for parenteral administration. In some embodiments, the parenteral formulations are prepared as an injectable formulation, e.g., for intravenous administration. For example, in some embodiments, when a compound of the disclosure is formulated for parenteral administration by injection (e.g., continuous infusion or bolus injection), the formulation can be in the form of a suspension, solution, or emulsion in an oily or aqueous vehicle, and such formulations can further comprise pharmaceutically necessary additives such as one or more stabilizing agents, suspending agents, dispersing agents, and the like. When a compound of the disclosure is to be injected parenterally, it can be, e.g., in the form of an isotonic sterile solution. A compound of the disclosure can also be in the form of a powder for reconstitution as an injectable formulation.

In some embodiments, the compound of the disclosure is formulated for oral administration. For example, in some embodiments, the compound of the disclosure is formulated as a tablet that comprises zero, one, two, or more of each of the following: emulsifier; surfactant, binder; disintegrant, glidant; and lubricant.

The pharmaceutical compositions containing the compound of the disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the compound of the disclosure into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the disclosure in the required amount in an appropriate solvent with one or a combination of ingredients enumerated above, as required, followed by filtered sterilization. Generally, dispersions are prepared by incorporating the active agent or compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the compound of the disclosure can be incorporated with excipients and used in the form of tablets, troches, or capsules. Oral compositions can also be prepared using a fluid carrier for use as a mouthwash, wherein the agent or compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the agents or compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents or compounds are formulated into ointments, salves, gels, or creams as generally known in the art.

In aspects, the compound of the disclosure is prepared with pharmaceutically acceptable carriers that will protect the agent or compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in unit dosage form for ease of administration and uniformity of dosage. Unit dosage form as used herein refers to physically discrete units suited as unitary dosages for the subject to be treated; each unit containing a predetermined quantity of active agent or compound calculated to produce the desired therapeutic effect in association with the required pharmaceutical carrier. The specification for the unit dosage forms of the application are dictated by and directly dependent on the unique characteristics of the compound of the disclosure and the particular therapeutic effect to be achieved.

The pharmaceutical compositions can be included in a container, pack, or dispenser together with instructions for administration.

Illustrative modes of administration for the compound of the disclosure includes systemic or local administration such as parenteral, oral, nasal, transdermal, subcutaneous, vaginal, buccal, rectal or topical administration modes. In some embodiments, the compound of the disclosure is administered orally. In some embodiments, the compound of the disclosure is administered as a tablet, capsule, caplet, solution, suspension, syrup, granule, bead, powder, or pellet.

Illustrative pharmaceutical compositions are tablets and gelatin capsules comprising a salt of the compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the salt such as cyclodextrin, hydroxypropyl-cyclodextrin, PEG400, and/or PEG200.

For preparing pharmaceutical compositions from the compound of the disclosure, or a salt or hydrate thereof, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed salt is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions and emulsions.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, intrathecal, subcutaneous or intramuscular form, and all using forms well known to those skilled in the pharmaceutical arts.

Pharmaceutical compositions can be prepared according to conventional mixing, granulating or coating methods, respectively, and the present pharmaceutical compositions can contain from about 0.1% to about 99%, from about 5% to about 90%, or from about 1% to about 20% of the disclosed free base or salt by weight or volume.

The pharmaceutical compositions containing the compound of the disclosure may further comprising one or more additional anti-cancer agents, including any of those disclosed herein.

All amounts of any component of an oral dosage form described herein, e.g., a tablet, that are indicated based on % w/w refer to the total weight of the oral dosage form, unless otherwise indicated.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Abbreviations:

Ac₂O acetic anhydride
Boc tert-butoxycarbonyl
Boc₂O di-tert-butyl dicarbonate
BSA bovine serum albumin
CbzCl benzyl chloroformate
DABCO 1,4-diazabicyclo[2.2.2]octane
DCE 1,2-dichloroethane
DIEA N,N-diisopropylethylamine
DMAP 4-dimethylaminopyridine
DMEM Dulbecco's Modified Eagle Medium
DMF dimethylformamide
DMSO dimethyl sulfoxide
dppf 1,1′-bis(diphenylphosphino)ferrocene
EtOAc ethyl acetate
EtOH ethanol
FBS fetal bovine serum
HATU 1-[bis(dimethylamino)methylene]-1H-1,2,3-triazolo[4,5-b]pyridinium 3-oxide hexafluorophosphate
i-PrOH or IPA isopropanol
KOAc potassium acetate
LiHMDS Lithium bis(trimethylsilyl)amide
m-CPBA meta-chloroperoxybenzoic acid
MOMCl methoxymethyl chloride
MOPS 3-(N-morpholino) propanesulfonic acid
NIS N-iodosuccinimide
OAc acetate group
PBS phosphate buffered saline
Pd(dppf)Cl₂ 1,1′-bis(diphenylphosphino)ferrocene]dichloropalladium (II)
PG protecting group
p-TsOH p-toluenesulfonic acid
SFC supercritical fluid chromatography
TBAF tetra-n-butylammonium fluoride
TBDPSCl tert-butyldiphenylchlorosilane
TBME tert-butyl methyl ether
TBS tris-buffered saline
TBS-T mixture of tris-buffered saline and polysorbate 20 (also known as Tween 20)
Tf₂O trifluoromethanesulfonic anhydride
TFA trifluoroacetic acid
THF tetrahydrofuran
THP tetrahydropyran
TLC thin layer chromatography

### Example 1: General Synthetic Schemes

The compounds of the present disclosure may be prepared according to, e.g. the routes described in Schemes 1-4:

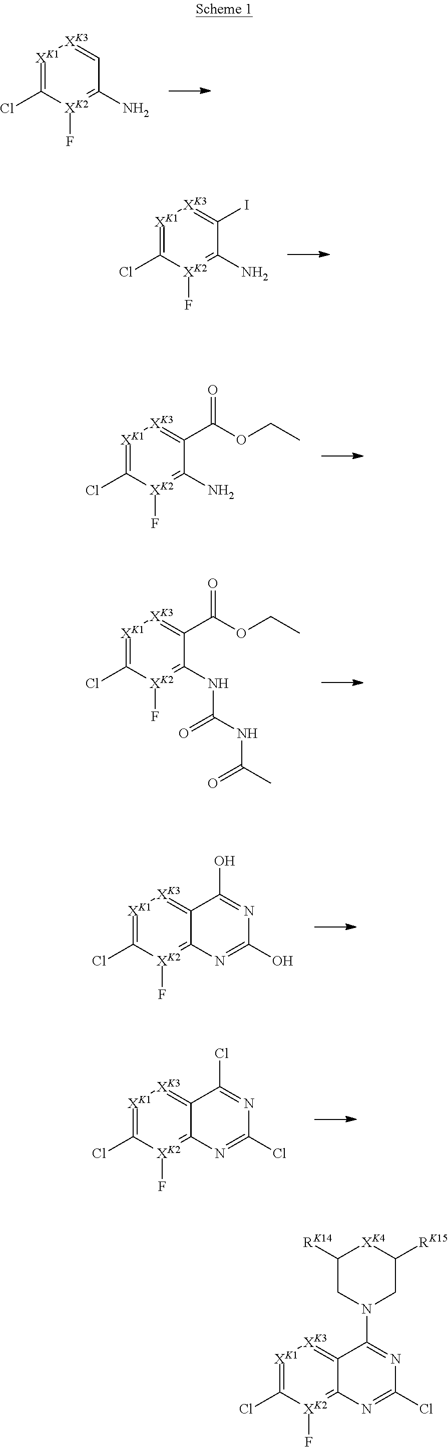

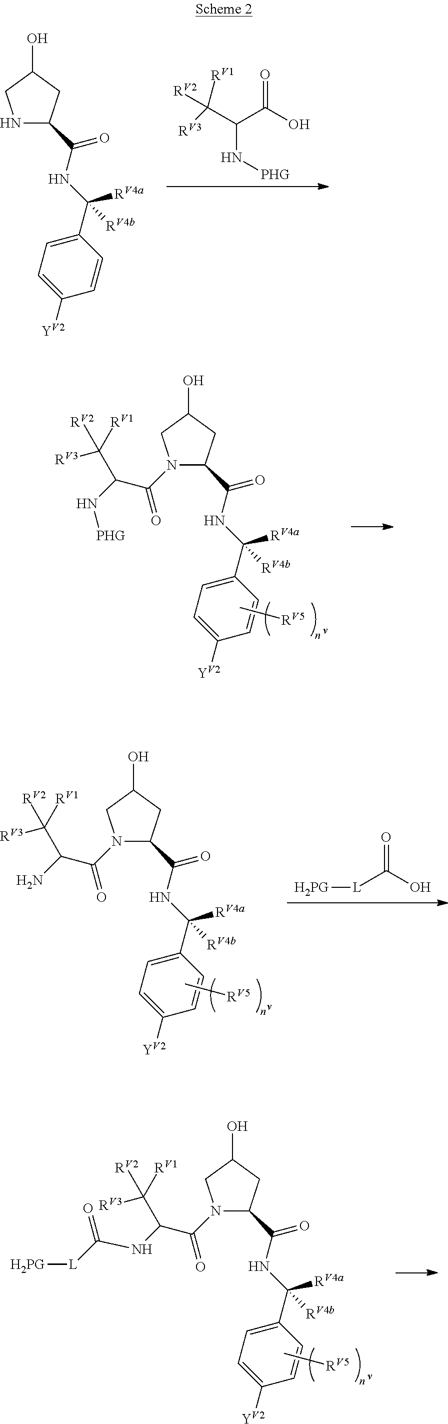

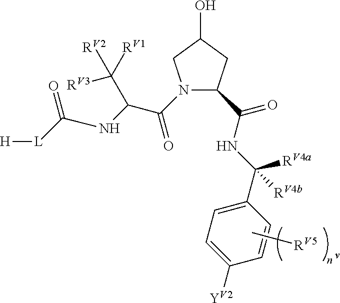

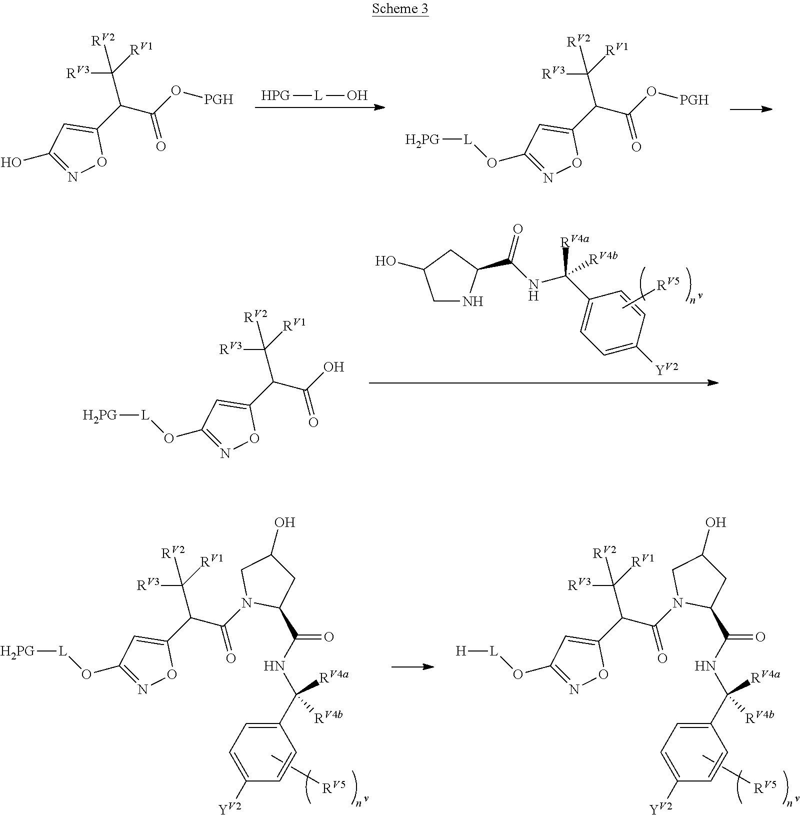

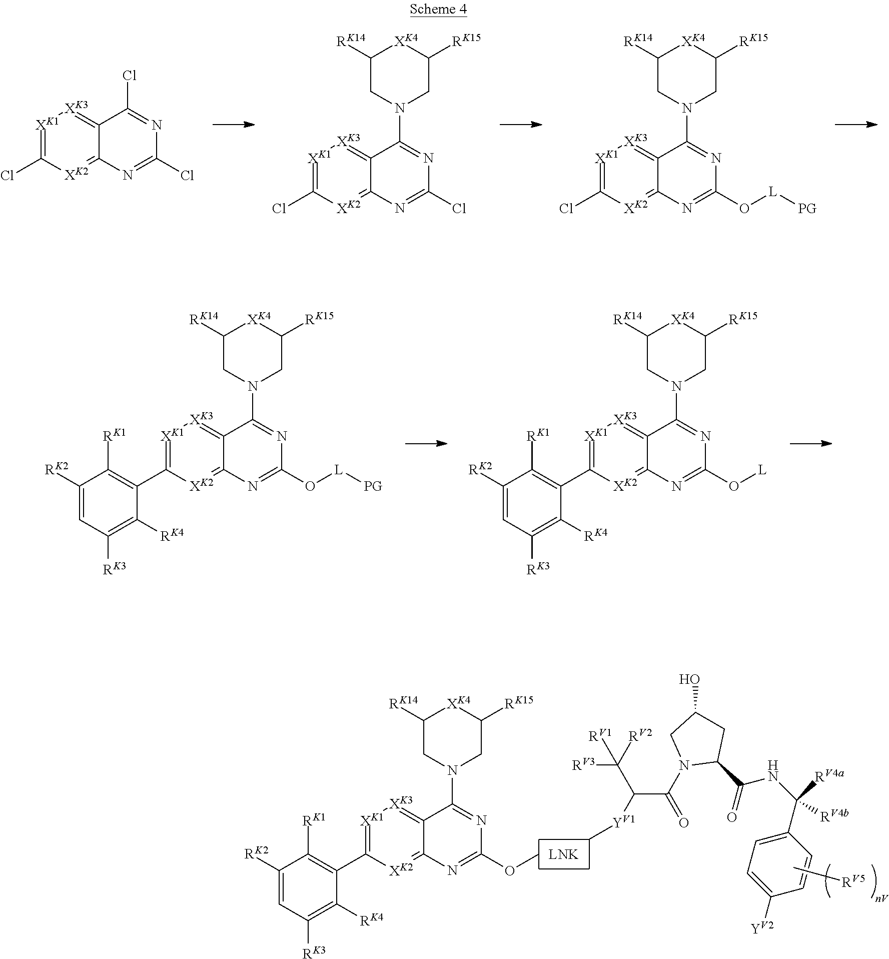

### Scheme 4

### Exemplary Synthesis of (2S,4R)-1-[(2S)-2-[[2-[[1-[2-[4-(3,8-diazabicyclo[3.2.1]octan-3-yl)-8-fluoro-7-(3-hydroxy-1-naphthyl)pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 23)

### Step 1: Preparation of 2-chloro-3-fluoro-5-iodo-pyridin-4-amine

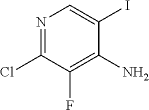

To a solution of 2-chloro-3-fluoro-pyridin-4-amine (2.00 g, 13.7 mmol, 1 eq) and NIS (3.68 g, 16.4 mmol, 1.2 eq) in CH₃CN (15 mL) was added p-TsOH (118 mg, 0.682 mmol, 0.05 eq), and the reaction mixture was stirred at 70° C. for 16 hours. The reaction mixture was diluted with EtOAc (40 mL), and the resulting mixture was washed with saturated aqueous Na₂CO₃ (2×30 mL), saturated aqueous Na₂SO₃ (40 mL), brine (30 mL), dried over anhydrous Na₂SO₄, filtered, and concentrated under reduced pressure to give 2-chloro-3-fluoro-5-iodo-pyridin-4-amine (3.63 g, 13.3 mmol, 98% yield) as a yellow solid. LC/MS (ESI) m/z: 272.8 [M+H]⁺. ¹H-NMR (400 MHz, DMSO-d₆) δ 8.09 (s, 1H), 6.67 (br s, 2H).

### Step 2: Preparation of ethyl 4-amino-6-chloro-5-fluoro-pyridine-3-carboxylate

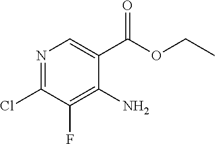

To a solution of 2-chloro-3-fluoro-5-iodo-pyridin-4-amine (3.63 g, 13.3 mmol, 1 eq) in EtOH (70 mL) were added triethylamine (4.85 g, 48.0 mmol, 6.68 mL, 3.6 eq) and Pd(PPh₃)₂Cl₂ (935 mg, 1.33 mmol, 0.1 eq), and the reaction mixture was stirred at 80° C. under CO (15 psi) (degassed under vacuum and purged with CO several times) for 16 hours. The reaction mixture was concentrated under reduced pressure to remove ˜70% of EtOH and then filtered. The filter cake was washed with TBME (2×30 mL) and then dried under reduced pressure to give ethyl 4-amino-6-chloro-5-fluoro-pyridine-3-carboxylate (3.40 g, crude) as a yellow solid. LC/MS (ESI) m/z: 219.0 [M+H]⁺.

### Step 3: Preparation of ethyl 6-chloro-5-fluoro-4-[(2,2,2-trichloroacetyl)carbamoylamino]pyridine-3-carboxylate

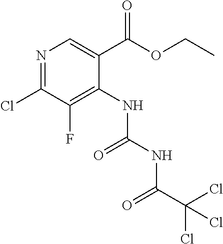 [SMILES: CCOC(=O)c1cnc(Cl)c(F)c1NC(=O)NC(=O)C(Cl)(Cl)Cl]

To a solution of ethyl 4-amino-6-chloro-5-fluoro-pyridine-3-carboxylate (3.40 g, 15.6 mmol, 1 eq) in THF (10 mL) was added 2,2,2-trichloroacetyl isocyanate (3.22 g, 17.1 mmol, 2.03 mL, 1.1 eq), and the reaction mixture was stirred at 20° C. under N₂ for 1 hour. The reaction mixture was concentrated under reduced pressure to give ethyl 6-chloro-5-fluoro-4-[(2,2,2-trichloroacetyl)carbamoylamino]pyridine-3-carboxylate (6.10 g, crude) as a brown solid. LC/MS (ESI) m/z: 408.1 [M+H]⁺.

### Step 4: Preparation of 7-chloro-8-fluoro-pyrido[4,3-d]pyrimidine-2,4-diol

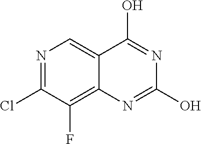 [SMILES: Oc1nc(O)c2cnc(Cl)c(F)c2n1]

To a solution of ethyl 6-chloro-5-fluoro-4-[(2,2,2-trichloroacetyl)carbamoylamino]pyridine-3-carboxylate (6.10 g, 15.0 mmol, 1 eq) in CH₃OH (55 mL) was added ammonia (7 M, 10.7 mL, 5 eq), and the reaction mixture was stirred at 20° C. for 1 hour. The reaction mixture was filtered, and the filter cake was washed with TBME (3×20 mL), then dried under reduced pressure to give 7-chloro-8-fluoro-pyrido[4,3-d]pyrimidine-2,4-diol (3.03 g, 14.06 mmol, 94% yield) as a white solid. LC/MS (ESI) m/z: 216.1 [M+H]⁺.

### Step 5: Preparation of 2,4,7-trichloro-8-fluoro-pyrido[4,3-d]pyrimidine

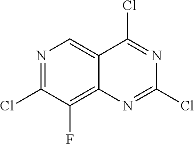 [SMILES: Fc1c(Cl)ncc2c(Cl)nc(Cl)nc12]

To a solution of 7-chloro-8-fluoro-pyrido[4,3-d]pyrimidine-2,4-diol (2.50 g, 11.6 mmol, 1 eq) in toluene (30 mL) were added DIEA (4.50 g, 34.8 mmol, 6.06 mL, 3 eq) and POCl₃ (8.89 g, 58.0 mmol, 5.39 mL, 5 eq), and the reaction mixture was stirred at 100° C. under N₂ for 1 hour. The reaction mixture was concentrated under reduced pressure to give 2,4,7-trichloro-8-fluoro-pyrido[4,3-d]pyrimidine (2.9 g, crude) as a yellow oil. LC/MS (ESI) m/z: 253.7 [M+H]⁺.

### Step 6: Preparation of tert-butyl 3-(2,7-dichloro-8-fluoro-pyrido[4,3-d]pyrimidin-4-yl)-3,8-diazabicyclo[3.2.1]octane-8-carboxylate

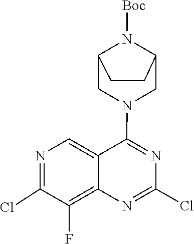 [SMILES: CC(C)(C)OC(=O)N1C2CCC1CN(c1nc(Cl)nc3c(F)c(Cl)ncc13)C2]

To a solution of 2,4,7-trichloro-8-fluoro-pyrido[4,3-d]pyrimidine (2.90 g, 11.5 mmol, 1 eq) in CH₂Cl₂ (50 mL) were added DIEA (7.42 g, 57.4 mmol, 5 eq) and tert-butyl(1S,5R)-3,8-diazabicyclo[3.2.1]octane-8-carboxylate (2.44 g, 11.5 mmol, 1 eq) at −40° C., and the reaction mixture was stirred at −40° C. under N₂ for 0.5 hour. The reaction mixture was poured into water (50 mL) and extracted with CH₂Cl₂ (3×50 mL). The combined organic layer was washed with brine (2×60 mL), dried over anhydrous Na₂SO₄, filtered, and concentrated under reduced pressure to give crude product. The crude product was purified by flash silica gel chromatography (eluent: 0˜25% EtOAc/petroleum ether) to give tert-butyl 3-(2,7-dichloro-8-fluoro-pyrido[4,3-d]pyrimidin-4-yl)-3,8-diazabicyclo[3.2.1]octane-8-carboxylate (2.47 g, 5.77 mmol, 50% yield) as a yellow solid confirmed). LC/MS (ESI) m/z: 428.2 [M+H]⁺. ¹H-NMR (400 MHz, DMSO-d₆) δ 9.06 (s, 1H), 4.60-4.40 (m, 2H), 4.31-4.22 (m, 2H), 3.80-3.59 (m, 2H), 1.85-1.74 (m, 2H), 1.66-1.57 (m, 2H), 1.46 (s, 9H).

### Step 7: Preparation of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate

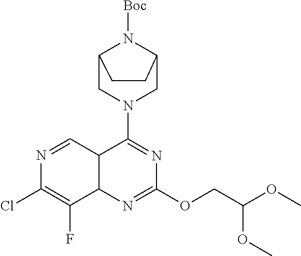 [SMILES: COC(COC1=NC2C(F)=C(Cl)N=CC2C(N2CC3CCC(C2)N3C(=O)OC(C)(C)C)=N1)OC]

To a solution of tert-butyl 3-(2,7-dichloro-8-fluoro-pyrido[4,3-d]pyrimidin-4-yl)-3,8-diazabicyclo[3.2.1]octane-8-carboxylate (500 mg, 1.17 mmol, 1 eq) and 2,2-dimethoxyethanol (186 mg, 1.75 mmol, 1.5 eq) in CH₃CN (10 mL) were added Cs₂CO₃ (456 mg, 1.40 mmol, 1.2 eq) and DABCO (13 mg, 0.17 mmol, 0.1 eq), and the reaction mixture was stirred at 20° C. under N₂ for 16 hours. The reaction mixture was filtered, and the filtrate was concentrated under reduced pressure to give crude product. The crude product was purified by flash silica gel chromatography (eluent: 0˜15% THF/petroleum ether) to give tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (685 mg, 1.38 mmol, 98% yield) as a yellow solid. LC/MS (ESI) m/z: 498.3 [M+H]⁺. ¹H-NMR (400 MHz, CDCl₃) δ 8.74 (s, 1H), 4.81 (t, J=5.6 Hz, 1H), 4.54-4.45 (m, 4H), 4.43-4.27 (m, 2H), 3.74-3.55 (m, 2H), 3.48 (s, 6H), 2.02-1.90 (m, 2H), 1.75-1.65 (m, 2H), 1.52 (s, 9H).

### Step 8: Preparation of tert-butyl 3-[[2-(2,2-dimethoxyethoxy)-8-fluoro-7-(3-hydroxy-1-naphthyl)pyrido[4,3-d]pyrimidin-4-yl]-3,8-diazabicyclo[3.2.1]octane-8-carboxylate

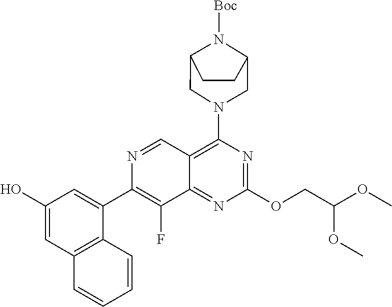 [SMILES: COC(COc1nc(N2CC3CCC(C2)N3C(=O)OC(C)(C)C)c2cnc(-c3cc(O)cc4ccccc34)c(F)c2n1)OC]

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (685 mg, 1.38 mmol, 1 eq) and 4-(4,4,5,5-tetramethyl-1,3,2-dioxaborolan-2-yl) naphthalen-2-ol (557 mg, 2.06 mmol, 1.5 eq) in dioxane (8 mL)/Water (2 mL) were added Cs₂CO₃ (896 mg, 2.75 mmol, 2 eq) and Pd(PPh₃)₄ (207 mg, 0.179 mmol, 0.13 eq), and the reaction mixture was stirred at 100° C. under N₂ (degassed under vacuum and purged with N₂ several times) for 16 hours. The reaction mixture was diluted with EtOAc (20 mL), then dried over anhydrous Na₂SO₄ and concentrated under reduced pressure to give crude product. The crude product was purified by flash silica gel chromatography (eluent: 0˜40% THF/petroleum ether) to give tert-butyl 3-[2-(2,2-dimethoxyethoxy)-8-fluoro-7-(3-hydroxy-1-naphthyl)pyrido[4,3-d]pyrimidin-4-yl]-3,8-diazabicyclo[3.2.1]octane-8-carboxylate (688 mg, 1.14 mmol, 83% yield) as a yellow solid. LC/MS (ESI) m/z: 606.5 [M+H]⁺.

### Step 9: Preparation of tert-butyl 3-[8-fluoro-7-(3-hydroxy-1-naphthyl)-2-(2-oxoethoxy)pyrido[4,3-d]pyrimidin-4-yl]-3,8-diazabicyclo[3.2.1]octane-8-carboxylate

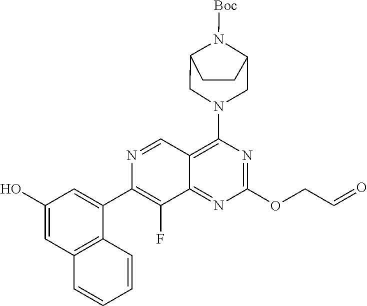 [SMILES: CC(C)(C)OC(=O)N1C2CCC1CN(c1nc(OCC=O)nc3c(F)c(-c4cc(O)cc5ccccc45)ncc13)C2]

A mixture of tert-butyl 3-[2-(2,2-dimethoxyethoxy)-8-fluoro-7-(3-hydroxy-1-naphthyl)pyrido[4,3-d]pyrimidin-4-yl]-3,8-diazabicyclo[3.2.1]octane-8-carboxylate (300 mg, 0.495 mmol, 1 eq) in acetone (0.62 mL)/HCl (12 M, 0.619 mL, 15 eq) was stirred at 20° C. for 20 minutes. A solution of NaHCO₃ (1.25 g, 14.9 mmol, 30 eq) in water (2 mL), Boc₂O (324 mg, 1.49 mmol, 3 eq), and THF (2 mL) were then added, and the resulting mixture was stirred at 20° C. for 0.5 hour. The reaction mixture was extracted with EtOAc (3×5 mL), and the combined organic layer was washed with brine (2×5 mL), dried over anhydrous Na₂SO₄, filtered, and concentrated under reduced pressure to give crude product. The crude product was purified by flash silica gel chromatography (eluent: 0˜50% THF/petroleum ether) to give tert-butyl 3-[8-fluoro-7-(3-hydroxy-1-naphthyl)-2-(2-oxoethoxy)pyrido[4,3-d]pyrimidin-4-yl]-3,8-diazabicyclo[3.2.1]octane-8-carboxylate (222 mg, 0.397 mmol, 80% yield) as a white solid. LC/MS (ESI) m/z: 560.4 [M+H]⁺.

### Step 10: Preparation of tert-butyl4-[(2-ethoxy-2-oxo-ethoxy)methyl]piperidine-1-carboxylate

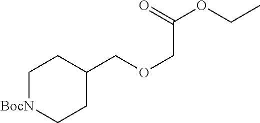 [SMILES: CCOC(=O)COCC1CCN(C(=O)OC(C)(C)C)CC1]

To a solution of tert-butyl 4-(hydroxymethyl) piperidine-1-carboxylate (2.00 g, 9.29 mmol, 1.00 eq) in CH₂Cl₂ (50 mL) were added diacetoxyrhodium (1.20 g, 4.64 mmol, 0.50 eq) and ethyl 2-diazoacetate (12.00 g, 92.90 mmol, 10 mL, 10.00 eq) at 0° C., and the reaction mixture was stirred at 25° C. for 12 hours. The solution was concentrated under reduced pressure and dried under vacuum. Purification by column chromatography on SiO₂ (0-20% EtOAc in petroleum ether) afforded tert-butyl 4-[(2-ethoxy-2-oxo-ethoxy)methyl]piperidine-1-carboxylate (1.50 g, 4.98 mmol, 54% yield) as a yellow oil. LC/MS (ESI) m/z: 202.2 [M-Boc+1]⁺.

### Step 11: Preparation of 2-[(1-tert-butoxycarbonyl-4-piperidyl)methoxy]acetic acid

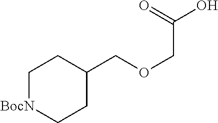 [SMILES: CC(C)(C)OC(=O)N1CCC(COCC(=O)O)CC1]

To a solution of tert-butyl 4-[(2-ethoxy-2-oxo-ethoxy)methyl]piperidine-1-carboxylate (1.50 g, 4.98 mmol, 1.00 eq) in THF (10 mL), CH₃OH (5 mL) and H₂O (5 mL) was added LiOH hydrate (700 mg, 14.93 mmol, 3.00 eq), and the reaction mixture was stirred at 25° C. for 12 hours. The reaction was acidified (pH=5) with dilute hydrochloric acid, and the resulting mixture was concentrated under reduced pressure to afford 2-[(1-tert-butoxycarbonyl-4-piperidyl)methoxy]acetic acid (1.00 g, 3.66 mmol, 74% yield) as a yellow oil.

Step 12: Preparation of tert-butyl4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]piperidine-1-carboxylate

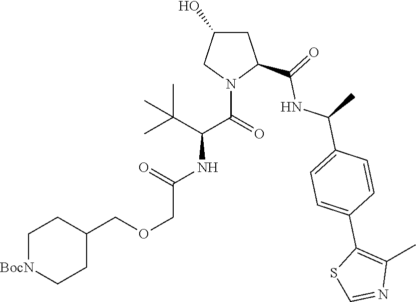 [SMILES: Cc1ncsc1-c1ccc([C@H](C)NC(=O)[C@@H]2C[C@@H](O)CN2C(=O)[C@@H](NC(=O)COCC2CCN(C(=O)OC(C)(C)C)CC2)C(C)(C)C)cc1]

To a solution of 2-[(1-tert-butoxycarbonyl-4-piperidyl)methoxy]acetic acid (115 mg, 0.42 mmol, 1.00 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (200 mg, 0.42 mmol, 1.00 eq, hydrochloride salt) in CH₂Cl₂ (4 mL) was added hydroxybenzotriazole (85 mg, 0.62 mmol, 1.50 eq), 1-(3-dimethylaminopropyl)-3-ethylcarbodiimide hydrochloride (120 mg, 0.62 mmol, 1.50 eq), and diisopropylethylamine (270 mg, 2.08 mmol, 5.00 eq), and the reaction mixture was stirred at 25° C. for 12 hours. The solution was concentrated under reduced pressure and dried under vacuum. Purification by thin layer chromatography (EtOAc/CH₃OH=20/1) afforded tert-butyl4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]piperidine-1-carboxylate (210 mg, 0.30 mmol, 72% yield) as a yellow oil. LC/MS (ESI) m/z: 700.4 [M+H]⁺.

Step 13: Preparation of (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide

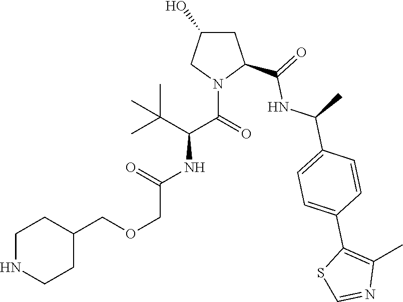 [SMILES: Cc1ncsc1-c1ccc([C@H](C)NC(=O)[C@@H]2C[C@@H](O)CN2C(=O)[C@@H](NC(=O)COCC2CCNCC2)C(C)(C)C)cc1]

To a solution of tert-butyl 4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]piperidine-1-carboxylate (100 mg, 0.14 mmol, 1.00 eq) in CH₂Cl₂ (2 mL) was added HCl (4 M in dioxane, 2 mL), and the reaction mixture was stirred at 25° C. for 0.5 hour. The solution was concentrated under reduced pressure and dried under vacuum to afford (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (90 mg, 0.14 mmol, 99% yield, hydrochloride salt) was obtained as a yellow solid.

Step 14: Preparation of tert-butyl 3-[8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)pyrido[4,3-d]pyrimidin-4-yl]-3,8-diazabicyclo[3.2.1]octane-8-carboxylate

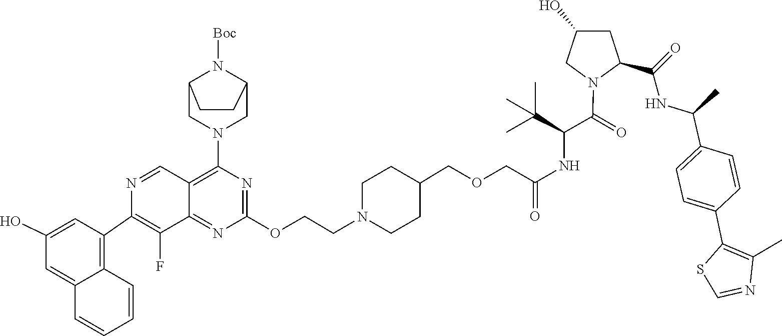 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CC5CCC(C4)N5C(=O)OC(C)(C)C)c4cnc(-c5cc(O)cc6ccccc56)c(F)c4n3)CC2)C(C)(C)C)cc1]

To a solution of tert-butyl 3-[8-fluoro-7-(3-hydroxy-1-naphthyl)-2-(2-oxoethoxy)pyrido[4,3-d]pyrimidin-4-yl]-3,8-diazabicyclo[3.2.1]octane-8-carboxylate (110 mg, 0.197 mmol, 1 eq) and (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (118 mg, 0.197 mmol, 1 eq) in CH₂Cl₂ (5 mL)/i-PrOH (0.5 mL) were added acetic acid (35 mg, 0.59 mmol, 3 eq) and 2-methylpyridine borane (105 mg, 0.983 mmol, 5 eq), and the reaction mixture was stirred at 20° C. for 0.5 hour. The reaction mixture was filtered, and the filtrate was purified by flash silica gel chromatography (eluent: 0˜10% CH₃OH/CH₂Cl₂) to give tert-butyl 3-[8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)pyrido[4,3-d]pyrimidin-4-yl]-3,8-diazabicyclo[3.2.1]octane-8-carboxylate (131 mg, 0.115 mmol, 58% yield) as a white solid. LC/MS (ESI) m/z: 1144.7 [M+H]⁺.

Step 15: Preparation of (2S,4R)-1-[(2S)-2-[[2-[[1-[2-[4-(3,8-diazabicyclo[3.2.1]octan-3-yl)-8-fluoro-7-(3-hydroxy-1-naphthyl)pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

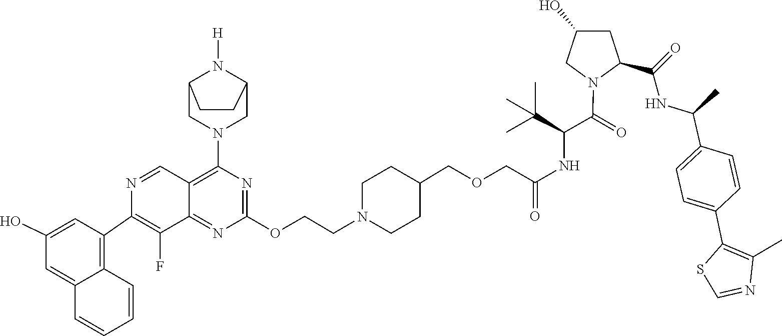 [SMILES: Cc1ncsc1-c1ccc([C@H](C)NC(=O)[C@@H]2C[C@@H](O)CN2C(=O)[C@@H](NC(=O)COCC2CCN(CCOc3nc(N4CC5CCC(C4)N5)c4cnc(-c5cc(O)cc6ccccc56)c(F)c4n3)CC2)C(C)(C)C)cc1]

To a solution of tert-butyl 3-[8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]-7-(3-hydroxy-1-naphthyl)pyrido[4,3-d]pyrimidin-4-yl]-3,8-diazabicyclo[3.2.1]octane-8-carboxylate (131 mg, 0.115 mmol, 1 eq) in CH₂Cl₂ (2 mL) was added TFA (2 mL), and the reaction mixture was stirred at 20° C. for 1 hour. The reaction mixture was concentrated under reduced pressure to give a residue. The residue was diluted with water (1 mL), and the resulting mixture was adjusted to pH=8 with saturated aqueous NaHCO₃. The resultant suspension was filtered, and the cake was diluted with EtOAc/THF (3×20 mL, 1/1). The organic layer was dried over anhydrous Na₂SO₄, filtered, and concentrated under reduced pressure to give crude product. The crude product was purified by prep-HPLC (gradient: 0-40% CH₃CN in water (0.225% formic acid)). The pure fractions were combined and dried by lyophilization to give (2S,4R)-1-[(2S)-2-[[2-[[1-[2-[4-(3,8-diazabicyclo[3.2.1]octan-3-yl)-8-fluoro-7-(3-hydroxy-1-naphthyl)pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (61.6 mg, 0.055 mmol, 48% yield, formic acid salt) as a white solid. LC/MS (ESI) m/z: 1043.4 [M+H]⁺. ¹H-NMR (400 MHz, CD₃OD) δ 9.15 (s, 1H), 8.89-8.84 (m, 1H), 8.47 (s, 1H), 7.76 (d, J=8.4 Hz, 1H), 7.53 (d, J=8.8 Hz, 1H), 7.46-7.35 (m, 5H), 7.29 (d, J=2.4 Hz, 1H), 7.27-7.20 (m, 2H), 5.01-4.95 (m, 1H), 4.80-4.70 (m, 4H), 4.69 (s, 1H), 4.60-4.50 (m, 3H), 4.09-3.70 (m, 8H), 3.59-3.36 (m, 5H), 2.89-2.68 (m, 2H), 2.46 (s, 3H), 2.26-2.17 (m, 1H), 2.05-1.88 (m, 8H), 1.63-1.42 (m, 5H), 1.05-0.99 (m, 9H).

Exemplary Synthesis of (2S,4R)-1-[(2R)-2-[3-[[1-[2-[4-(3,8-diazabicyclo[3.2.1]octan-3-yl)-8-fluoro-7-(3-hydroxy-1-naphthyl)pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 22)

Step 1: Preparation of tert-butyl 4-[[5-(1-methoxycarbonyl-2-methyl-propyl)isoxazol-3-yl]oxymethyl]piperidine-1-carboxylate

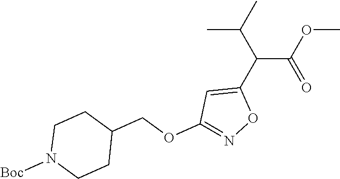 [SMILES: COC(=O)C(c1cc(OCC2CCN(C(=O)OC(C)(C)C)CC2)no1)C(C)C]

To a solution of tert-butyl 4-(hydroxymethyl) piperidine-1-carboxylate (1.30 g, 6.02 mmol, 1.2 eq) and methyl 2-(3-hydroxyisoxazol-5-yl)-3-methyl-butanoate (1 g, 5.02 mmol, 1 eq) in THP (10 mL) were added Ph₃P (1.58 g, 6.02 mmol, 1.2 eq) and diisopropyl azodicarboxylate (1.22 g, 6.02 mmol, 1.17 mL, 1.2 eq), and the reaction mixture was stirred at 25° C. for 12 hours. The reaction mixture was concentrated under reduced pressure, and the resulting residue was purified by flash silica gel chromatography (0-60% EtOAc in petroleum ether) to afford tert-butyl 4-[[5-(1-methoxycarbonyl-2-methyl-propyl)isoxazol-3-yl]oxymethyl]piperidine-1-carboxylate (1.8 g, 4.54 mmol, 90% yield) as a white solid. LC/MS (ESI) m/z: 297.2 [M+H]⁺; ¹H-NMR (400 MHz, CDCl₃) δ 5.88 (s, 1H), 4.21-4.10 (m, 2H), 4.07 (d, J=6.4 Hz, 2H), 3.73 (s, 3H), 3.49 (d, J=8.7 Hz, 1H), 2.82-2.62 (m, 2H), 2.35 (qd, J=7.1, 14.2 Hz, 1H), 1.96 (br d, J=3.4 Hz, 1H), 1.77 (br d, J=12.8 Hz, 2H), 1.46 (s, 9H), 1.29-1.22 (m, 2H), 1.00 (d, J=6.7 Hz, 3H), 0.93 (d, J=6.7 Hz, 3H).

Step 2: Preparation of 2-[3-[(1-tert-butoxycarbonyl-4-piperidyl)methoxy]isoxazol-5-yl]-3-methyl-butanoic acid

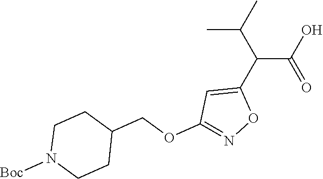 [SMILES: CC(C)C(C(=O)O)c1cc(OCC2CCN(C(=O)OC(C)(C)C)CC2)no1]

To a solution of tert-butyl 4-[[5-(1-methoxycarbonyl-2-methyl-propyl)isoxazol-3-yl]oxymethyl]piperidine-1-carboxylate (1.8 g, 4.54 mmol, 1 eq) in THF (8 mL), CH₃OH (5 mL), and H₂O (3 mL) was added LiOH monohydrate (544 mg, 22.70 mmol, 5 eq), and the reaction mixture was stirred at 25° C. for 1 hours. The reaction mixture was acidified (pH=3) by addition of 1 M hydrochloric acid, and the resulting precipitate was filtered to afford crude 2-[3-[(1-tert-butoxycarbonyl-4-piperidyl)methoxy]isoxazol-5-yl]-3-methyl-butanoic acid (1.9 g) as a white solid.

Step 3: Preparation of tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate

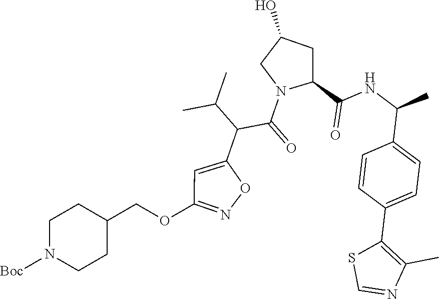 [SMILES: Cc1ncsc1-c1ccc([C@H](C)NC(=O)[C@@H]2C[C@@H](O)CN2C(=O)C(c2cc(OCC3CCN(C(=O)OC(C)(C)C)CC3)no2)C(C)C)cc1]

To a solution of 2-[3-[(1-tert-butoxycarbonyl-4-piperidyl)methoxy]isoxazol-5-yl]-3-methyl-butanoic acid (1.0 g, 2.61 mmol, 1.0 eq) in CH₂Cl₂ (30 mL) were added DIEA (2.3 mL, 13.07 mmol, 5.0 eq) and HATU (1.3 g, 3.40 mmol, 1.3 eq), and the reaction mixture was stirred at 25° C. under N₂ for 10 minutes. (2S,4R)-4-Hydroxy-N-[(1S)-1-[4-(4-methylthiazol-5-yl)phenyl]ethyl]pyrrolidine-2-carboxamide (962 mg, 2.61 mmol, 1.0 eq, HCl) was then added, and the reaction mixture was stirred at 25° C. under N₂ for 2 hours. The reaction mixture was poured onto water (30 mL) and the organic layer was separated. The aqueous layer was further extracted with CH₂Cl₂ (3×30 mL). The combined organic layer was washed with brine (50 mL), dried over anhydrous Na₂SO₄, filtered, and concentrated under reduced pressure to give crude product, which was purified by flash chromatography on silica gel (gradient: (0˜58% THF in petroleum ether) to afford tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate (1.3 g, 1.55 mmol, 59% yield) as a yellow solid. LC/MS (ESI) m/z: 696.4 [M+H]⁺. This material was separated by SFC (column: DAICEL CHIRALPAK AD (250 mm*50 mm, 10 um); mobile phase: 35% isopropanol in water (0.1% NH₃)) to afford tert-butyl4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate and tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate.

tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate ¹H-NMR (400 MHz, CDCl₃) δ 8.68 (s, 1H), 7.48 (br d, J=7.5 Hz, 1H), 7.39 (q, J=8.3 Hz, 4H), 5.88 (s, 1H), 5.07 (t, J=7.2 Hz, 1H), 4.67-4.60 (m, 2H), 4.19-4.02 (m, 4H), 3.80 (dd, J=5.1, 10.5 Hz, 1H), 3.60 (dd, J=3.7, 10.4 Hz, 1H), 3.53-3.48 (m, 1H), 2.78-2.66 (m, 2H), 2.58-2.47 (m, 4H), 2.46-2.35 (m, 1H), 1.96 (ddd, J=5.0, 8.0, 12.8 Hz, 2H), 1.75 (br d, J=12.1 Hz, 3H), 1.50 (d, J=7.0 Hz, 3H), 1.46 (s, 9H), 1.28-1.19 (m, 2H), 1.05 (d, J=6.6 Hz, 3H), 0.93 (d, J=6.7 Hz, 3H).

tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate ¹H-NMR (400 MHz, CDCl₃) δ 8.70-8.68 (m, 1H), 7.42-7.39 (m, 2H), 7.36-7.32 (m, 3H), 5.86 (s, 1H), 5.02-4.94 (m, 1H), 4.78 (dd, J=4.3, 8.3 Hz, 1H), 4.66 (quin, J=5.4 Hz, 1H), 4.16-4.09 (m, 1H), 4.08-4.01 (m, 2H), 3.74-3.68 (m, 1H), 3.60-3.48 (m, 2H), 2.78-2.62 (m, 3H), 2.52-2.41 (m, 1H), 2.02-1.93 (m, 2H), 1.75 (br d, J=9.9 Hz, 6H), 1.46 (s, 9H), 1.38 (d, J=7.0 Hz, 3H), 1.30-1.18 (m, 3H), 1.06 (d, J=6.6 Hz, 3H), 0.94 (d, J=6.7 Hz, 3H)

Step 4: Preparation of (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carb oxamide

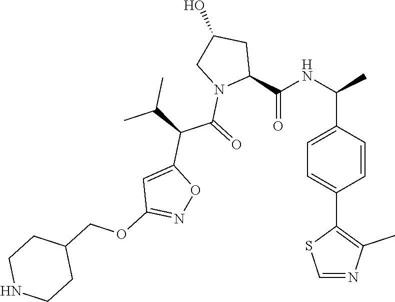 [SMILES: Cc1ncsc1-c1ccc([C@H](C)NC(=O)[C@@H]2C[C@@H](O)CN2C(=O)[C@@H](c2cc(OCC3CCNCC3)no2)C(C)C)cc1]

To a solution of tert-butyl 4-(((5-((R)-1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)oxy)methyl) piperidine-1-carboxylate (720 mg, 1.03 mmol, 1 eq) in CH₂Cl₂ (5 mL) was added HCl (4N in dioxane, 3.6 mL, 14 eq), and the reaction mixture was stirred at 25° C. for 1 hour. The mixture was concentrated, H₂O (5 mL) was then added, and the pH of the resulting aqueous mixture was adjusted to pH ˜8 by addition of saturated aqueous NaHCO₃. The aqueous mixture was extracted with CH₂Cl₂/CH₃OH (3×20 mL, V/V: 10/1), and the combined organic extract was dried over Na₂SO₄, filtered, and concentrated under reduced pressure to afford (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (610 mg, crude) as a white solid. LC/MS (ESI) m/z: 596.3 [M+H]⁺.

Step 5: Preparation of tert-butyl 3-[8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]-7-(3-hydroxy-1-naphthyl)pyrido[4,3-d]pyrimidin-4-yl]-3,8-diazabicyclo[3.2.1]octane-8-carboxylate

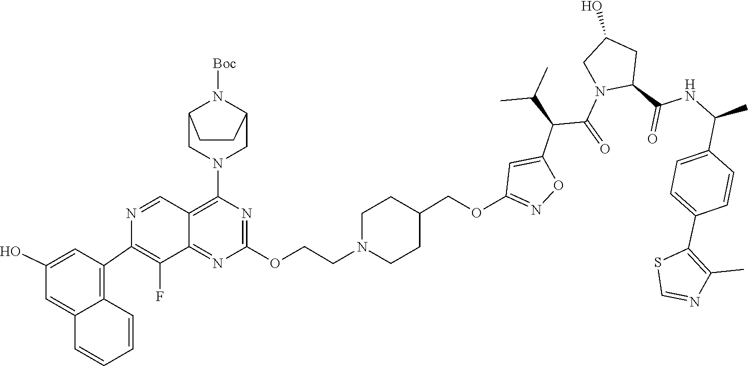 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6C(=O)OC(C)(C)C)c5cnc(-c6cc(O)cc7ccccc67)c(F)c5n4)CC3)no2)C(C)C)cc1]

To a solution of tert-butyl 3-[8-fluoro-7-(3-hydroxy-1-naphthyl)-2-(2-oxoethoxy)pyrido[4,3-d]pyrimidin-4-yl]-3,8-diazabicyclo[3.2.1]octane-8-carboxylate (110 mg, 197 mmol, 1 eq) and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (117 mg, 0.197 mmol, 1 eq) in CH₂Cl₂ (5 mL)/i-PrOH (0.5 mL) were added acetic acid (35 mg, 0.59 mmol, 3 eq) and 2-methylpyridine borane (105 mg, 0.983 mmol, 5 eq), and the reaction mixture was stirred at 20° C. for 0.5 hour. The reaction mixture was filtered, and the filtrate was purified by flash silica gel chromatography (eluent: 0˜10% CH₃OH/CH₂Cl₂) to give tert-butyl 3-[8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]-7-(3-hydroxy-1-naphthyl)pyrido[4,3-d]pyrimidin-4-yl]-3,8-diazabicyclo[3.2.1]octane-8-carboxylate (127 mg, 0.111 mmol, 57% yield) as a yellow solid). LC/MS (ESI) m/z: 1139.5 [M+H]⁺.

Step 5: Preparation of (2S,4R)-1-[(2R)-2-[3-[[1-[2-[4-(3,8-diazabicyclo[3.2.1]octan-3-yl)-8-fluoro-7-(3-hydroxy-1-naphthyl)pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

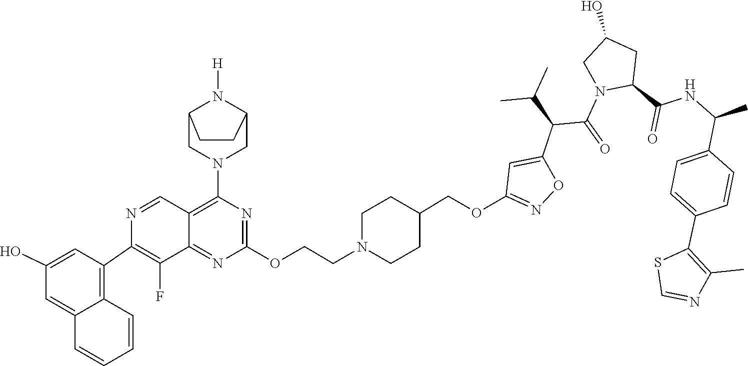 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc7ccccc67)c(F)c5n4)CC3)no2)C(C)C)cc1]

To a solution of tert-butyl 3-[8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]-7-(3-hydroxy-1-naphthyl)pyrido[4,3-d]pyrimidin-4-yl]-3,8-diazabicyclo[3.2.1]octane-8-carboxylate (127 mg, 0.111 mmol, 1 eq) in CH₂Cl₂ (2 mL) was added TFA (2 mL), and the reaction mixture was stirred at 20° C. for 1 hour. The reaction mixture was concentrated under reduced pressure to give a residue. The residue was diluted with water (1 mL), and the resulting mixture was adjusted to pH=8 with saturated aqueous NaHCO₃. The resultant suspension was filtered, and the cake was diluted with EtOAc/THF (3×20 mL, 1/1). The organic layer was dried over anhydrous Na₂SO₄, filtered, and concentrated under reduced pressure to give crude product. The crude product was purified by prep-HPLC (gradient: 0-40% CH₃CN in water (0.225% formic acid)). The pure fractions were combined and dried by lyophilization to give (2S,4R)-1-[(2R)-2-[3-[[1-[2-[4-(3,8-diazabicyclo[3.2.1]octan-3-yl)-8-fluoro-7-(3-hydroxy-1-naphthyl)pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (81.8 mg, 0.072 mmol, 65% yield, formic acid salt) as a white solid. LC/MS (ESI) m/z: 1039.4 [M+H]⁺. ¹H-NMR (400 MHz, CD₃OD) δ 9.15 (s, 1H), 8.88-8.85 (m, 1H), 8.48 (s, 1H), 7.76 (d, J=8.4 Hz, 1H), 7.53 (d, J=8.4 Hz, 1H), 7.47-7.36 (m, 5H), 7.31-7.28 (m, 1H), 7.26-7.20 (m, 2H), 6.00-5.92 (m, 1H), 5.03 (q, J=7.2 Hz, 1H), 4.78-4.71 (m, 5H), 4.51 (t, J=8.4 Hz, 1H), 4.08 (d, J=6.0 Hz, 2H), 3.97-3.90 (m, 2H), 3.87-3.72 (m, 3H), 3.67 (d, J=10.0 Hz, 1H), 3.60 (d, J=10.8 Hz, 1H), 3.49-3.41 (m, 2H), 3.28-3.21 (m, 2H), 2.75-2.62 (m, 2H), 2.50-2.45 (m, 3H), 2.42-2.32 (m, 1H), 2.22-2.12 (m, 1H), 2.03-1.89 (m, 8H), 1.63-1.47 (m, 5H), 1.05 (d, J=6.5 Hz, 3H), 0.92-0.85 (m, 3H).

Exemplary Synthesis of (2S,4R)-1-[(2S)-2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 21)

Step 1: Preparation of 8-(2-triisopropylsilylethynyl) naphthalene-1,3-diol

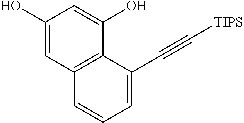

To a solution of naphthalene-1,3-diol (9.17 g, 57.25 mmol, 1.0 eq) and 2-bromoethynyl(triisopropyl)silane (17.95 g, 68.70 mmol, 1.2 eq) in anhydrous 1,4-dioxane (120 mL) were added KOAc (11.24 g, 114.50 mmol, 2.0 eq) and (1-isopropyl-4-methyl-benzene) ruthenium dichloride dimer (3.51 g, 5.73 mmol, 0.10 eq), and the reaction mixture was stirred at 110° C. for 16 hours. The mixture was filtered through celite pad, and the resulting filtrate was concentrated, then diluted with EtOAc (400 mL). The organic phase was washed with brine (60 mL), dried over anhydrous sodium sulfate, filtered, and concentrated. The resulting residue was purified by flash chromatography on silica gel (gradient: 0˜5% EtOAc in petroleum ether) afford 8-(2-triisopropylsilylethynyl) naphthalene-1,3-diol (12.12 g, 30.25 mmol, 53% yield) as a yellow solid.

Step 2: Preparation of 3-(methoxymethoxy)-8-(2-triisopropylsilylethynyl) naphthalen-1-ol

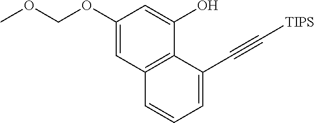

To a solution of 8-(2-triisopropylsilylethynyl) naphthalene-1,3-diol (12.12 g, 30.25 mmol, 1.0 eq) in anhydrous CH₂Cl₂ (120 mL) at 0° C. were added DIEA (13.80 g, 106.77 mmol, 3.53 eq) and MOMCl (4.30 g, 53.39 mmol, 1.76 eq) dropwise, and the reaction mixture was stirred at 0° C. for 40 minutes. The reaction mixture was quenched by water (100 mL) at 0° C., and then extracted with CH₂Cl₂ (3×100 mL). The combined organic extract was washed with brine (80 mL), dried over anhydrous sodium sulfate, filtered, and concentrated. The resulting residue was purified by flash chromatography on silica gel (gradient: 0˜2% EtOAc in petroleum ether) to afford 3-(methoxymethoxy)-8-(2-triisopropylsilylethynyl) naphthalen-1-ol (8.50 g, 22.10 mmol, 62% yield) as yellow oil. ¹H-NMR (400 MHz, CDCl₃) δ 9.25 (s, 1H), 7.69 (dd, J=8.4, 0.8, Hz, 1H), 7.50 (dd, J=7.2, 1.2, Hz, 1H), 7.31 (dd, J=8.0, 7.2, Hz, 1H), 6.98 (d, J=2.4 Hz, 1H), 6.78 (d, J=2.4 Hz, 1H), 5.27 (s, 2H), 3.51 (s, 3H), 1.22-1.16 (m, 21H).

Step 3: Preparation of [3-(methoxymethoxy)-8-(2-triisopropylsilylethynyl)-1-naphthyl]acetate

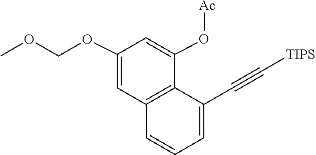

To a solution of 3-(methoxymethoxy)-8-(2-triisopropylsilylethynyl) naphthalen-1-ol (4.5 g, 11.70 mmol, 1.0 eq) in anhydrous CH₂Cl₂ (45 mL) at 0° C. were added DIEA (3.78 g, 29.25 mmol, 2.5 eq) and acetyl chloride (1.38 g, 17.55 mmol, 1.5 eq), and the reaction mixture was stirred at 0° C. for 1 hour. The reaction mixture was quenched with H₂O (30 mL) at 0° C. and then extracted with CH₂Cl₂ (3×40 mL). The combined organic extract was washed with brine (50 mL), dried over anhydrous sodium sulfate, filtered, and concentrated. The resulting residue was purified by flash chromatography on silica gel (gradient: 0˜3% EtOAc in petroleum ether) to afford crude [3-(methoxymethoxy)-8-(2-triisopropylsilylethynyl)-1-naphthyl]acetate (5.27 g) as yellow oil. LC/MS (ESI) m/z: 427.4 [M+H]⁺.

Step 4: Preparation of [8-ethynyl-3-(methoxymethoxy)-1-naphthyl]acetate

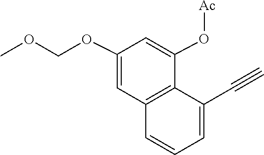 [SMILES: C#Cc1cccc2cc(OCOC)cc(OC(C)=O)c12]

To a solution of [3-(methoxymethoxy)-8-(2-triisopropylsilylethynyl)-1-naphthyl]acetate (4.99 g, 11.70 mmol, 1.0 eq) in DMF (50 mL) was added CsF (12.44 g, 81.88 mmol, 7.0 eq), and the reaction mixture was stirred at 20° C. for 2 hours. The reaction mixture was diluted with methyl tert-butyl ether (500 mL), and the organic phase was washed with water (8×50 mL) followed by brine (50 mL), dried over anhydrous sodium sulfate, filtered, and concentrated. The resulting residue was purified by flash chromatography on silica gel (gradient: 0˜5% EtOAc in petroleum ether) to afford [8-ethynyl-3-(methoxymethoxy)-1-naphthyl]acetate (1.69 g, 6.13 mmol, 52% yield) as a black-brown solid. ¹H-NMR (400 MHz, CDCl₃) δ 7.76 (d, J=7.6 Hz, 1H), 7.65 (dd, J=7.2, 1.2 Hz, 1H), 7.38 (dd, J=8.0, 7.2 Hz, 1H), 7.34 (d, J=2.4 Hz, 1H), 6.98 (d, J=2.4 Hz, 1H), 5.29 (s, 2H), 3.53 (s, 3H), 3.39 (s, 1H), 2.42 (s, 3H).

Step 5: Preparation of [8-ethyl-3-(methoxymethoxy)-1-naphthyl]acetate

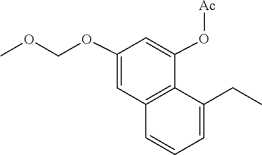 [SMILES: CCc1cccc2cc(OCOC)cc(OC(C)=O)c12]

To a solution of [8-ethynyl-3-(methoxymethoxy)-1-naphthyl]acetate (1.69 g, 6.13 mmol) in CH₃OH (30 mL) and THF (10 mL) was added 10% Pd/C (100 mg) under argon, and the reaction mixture was stirred at 25° C. for 4 hours under H₂ (15 psi). The mixture was filtered through celite pad and rinsed with EtOAc (3×40 mL). The filtrate was concentrated to dryness to afford [8-ethyl-3-(methoxymethoxy)-1-naphthyl]acetate (1.63 g, 5.94 mmol, 95% yield) as a yellow oil. LC/MS (ESI) m/z: 275.0 [M+H]⁺.

Step 6: Preparation of 8-ethyl-3-(methoxymethoxy) naphthalen-1-ol

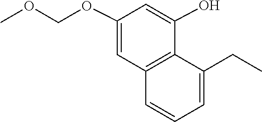 [SMILES: CCc1cccc2cc(OCOC)cc(O)c12]

To a solution of [8-ethyl-3-(methoxymethoxy)-1-naphthyl]acetate (1.63 g, 5.94 mmol, 1.0 eq) in THF (15 mL) and H₂O (5 mL) was added LiOH monohydrate (1.25 g, 29.71 mmol, 5.0 eq), and the reaction mixture was stirred at 25° C. for 3 hours. The reaction mixture was concentrated to remove the organic solvent, and the pH of the residual aqueous was adjusted to 5 by addition of 3N hydrochloric acid at 0° C. The resulting aqueous mixture w and was extracted with EtOAc (3×50 mL), and the combined organic extract was washed with brine (50 mL), dried over anhydrous sodium sulfate, filtered, and concentrated. The resulting residue was purified by flash chromatography on silica gel (gradient: 0-5% EtOAc in petroleum ether) to afford 8-ethyl-3-(methoxymethoxy) naphthalen-1-ol (925 mg, 3.98 mmol, 67% yield) as a yellow solid. LC/MS (ESI) m/z: 233.1 [M+H]⁺.

Step 7: Preparation of [8-ethyl-3-(methoxymethoxy)-1-naphthyl]trifluoromethanesulfonate

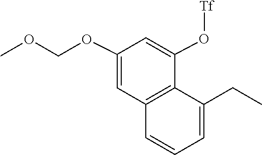

To a solution of 8-ethyl-3-(methoxymethoxy) naphthalen-1-ol (920 mg, 3.96 mmol, 1.0 eq) in anhydrous CH₂Cl₂ (12 mL) at −40° C. were added DIEA (2.56 g, 19.80 mmol, 5.0 eq) and trifluoromethanesulfonic anhydride (1.68 g, 5.94 mmol, 1.5 eq) dropwise, and the reaction mixture was stirred at −40° C. for 30 minutes under N₂. The reaction mixture was quenched with water (10 mL) at −40° C., warmed to room temperature, and then extracted with CH₂Cl₂ (3×30 mL). The combined organic extract was washed with brine (40 mL), dried over anhydrous sodium sulfate, filtered, and concentrated. The resulting residue was purified by flash chromatography on silica gel (gradient: 0˜1% EtOAc in petroleum ether) to afford [8-ethyl-3-(methoxymethoxy)-1-naphthyl]trifluoromethanesulfonate (1.23 g, 3.27 mmol, 83% yield) as a yellow oil.

Step 8: Preparation of 2-[8-ethyl-3-(methoxymethoxy)-1-naphthyl]-4,4,5,5-tetramethyl-1,3,2-dioxaborolane

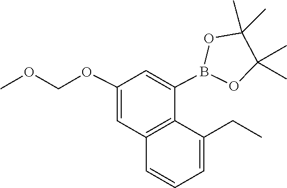

A mixture of [8-ethyl-3-(methoxymethoxy)-1-naphthyl]trifluoromethanesulfonate (1.23 g, 3.38 mmol, 1.0 eq), bis(pinacolato)diboron (1.89 g, 7.43 mmol, 2.2 eq), Pd(dppf) Cl₂ (494 mg, 0.675 mmol, 0.20 eq), and KOAc (1.16 g, 11.82 mmol, 3.5 eq) in anhydrous 1,4-dioxane (12 mL) was degassed and purged with N₂ (3×), then stirred at 110° C. for 16 hours under N₂ atmosphere. The reaction mixture was filtered, and the filter cake was washed with methyl tert-butyl ether (3×40 mL). The combined organic phase was washed with brine (30 mL), dried over anhydrous sodium sulfate, filtered, and concentrated. The resulting residue was purified by flash chromatography on silica gel (gradient: 0˜2% EtOAc in petroleum ether) to afford 2-[8-ethyl-3-(methoxymethoxy)-1-naphthyl]-4,4,5,5-tetramethyl-1,3,2-dioxaborolane (670 mg, 1.86 mmol, 55% yield) as yellow oil. LC/MS (ESI) m/z: 343.2 [M+H]⁺.

Step 9: Preparation of tert-butyl 3-[[2-(2,2-dimethoxyethoxy)-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

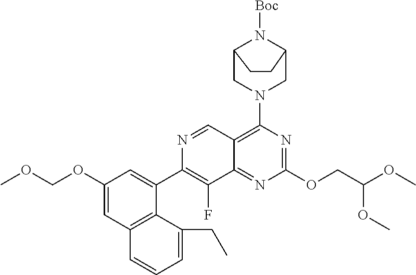

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (1.6 g, 3.21 mmol, 1 eq) and 2-[8-ethyl-3-(methoxymethoxy)-1-naphthyl]-4,4,5,5-tetramethyl-1,3,2-dioxaborolane (1.28 g, 3.73 mmol, 1.16 eq) in dioxane (15 mL) and H₂O (3 mL) were added Cs₂CO₃ (2.62 g, 8.03 mmol, 2.5 eq) and [1,1′-bis(di-tert-butylphosphino)ferrocene]dichloropalladium (II) (418.8 mg, 0.643 mmol, 0.2 eq), and the reaction mixture was stirred at 110° C. for 16 hours under N₂. The mixture was diluted with EtOAc (150 mL), and the combined organic phase was washed with brine (30 mL), dried over anhydrous sodium sulfate, filtered, and concentrated in vacuo. The resulting the residue was purified by flash chromatography on silica gel (gradient: 10˜30% EtOAc in petroleum ether) to afford tert-butyl 3-[[2-(2,2-dimethoxyethoxy)-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (0.922 g, 1.28 mmol, 40% yield) as a brown solid. LC/MS (ESI) m/z: 678.4 [M+H]⁺.

Step 10: Preparation of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate

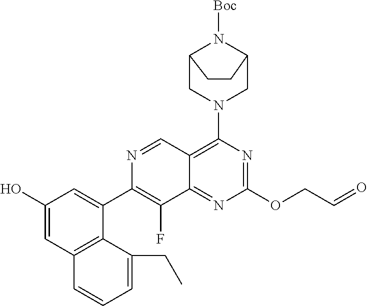 [SMILES: CCc1cccc2cc(O)cc(-c3ncc4c(N5CC6CCC(C5)N6C(=O)OC(C)(C)C)nc(OCC=O)nc4c3F)c12]

To a solution of tert-butyl 3-[[2-(2,2-dimethoxyethoxy)-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (100 mg, 0.148 mmol, 1 eq) in acetone (0.37 mL) was added concentrated HCl (12 M, 0.37 mL, 30 eq) dropwise, and the reaction mixture was stirred at 20° C. for 5 minutes (9 batches were conducted in total). Saturated aqueous NaHCO₃ was then added until pH=8, and the resulting mixture was filtered, washing with water (10 mL) and petroleum ether (10 mL) to give 2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyacetaldehyde (640 mg crude) as a yellow solid. Boc₂O (343.8 mg, 1.58 mmol, 1.2 eq) and a solution of NaHCO₃ (330.8 mg, 3.94 mmol, 3 eq) in H₂O (2.5 mL) were then added, and the reaction mixture was stirred at 20° C. for 2 hours. The mixture was diluted with EtOAc (120 mL), and the organic phase was washed with brine (20 mL), dried over anhydrous sodium sulfate, filtered, and concentrated. The resulting residue was purified by flash chromatography on silica gel (gradient: 10˜50% EtOAc in petroleum ether) to afford tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (430 mg, 46% yield) as a yellow solid. LC/MS (ESI) m/z: 588.4 [M+H]⁺.

Step 11: Preparation of tert-butyl 3-[7-(8-ethyl-3-hydroxy-1-naphthyl)-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperid yl]ethoxy]pyrido[4,3-d]pyrimidin-4-yl]-3,8-diazabicyclo[3.2.1]octane-8-carboxylate

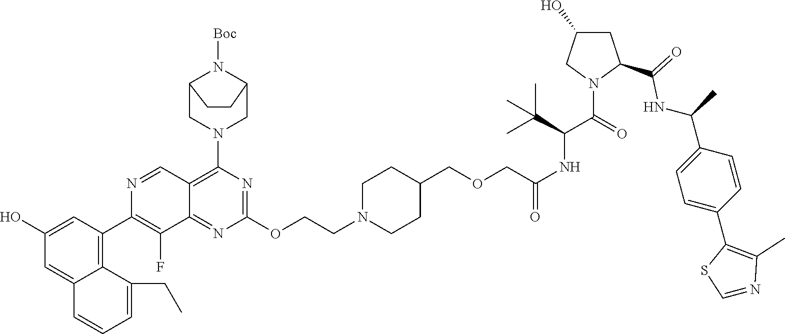 [SMILES: CCc1cccc2cc(O)cc(-c3ncc4c(N5CC6CCC(C5)N6C(=O)OC(C)(C)C)nc(OCCN5CCC(COCC(=O)N[C@H](C(=O)N6C[C@H](O)C[C@H]6C(=O)N[C@@H](C)c6ccc(-c7scnc7C)cc6)C(C)(C)C)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (130 mg, 0.221 mmol, 1 eq) and (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (132.7 mg, 0.221 mmol, 1 eq) in CH₂Cl₂ (2 mL) and IPA (2 mL) were added acetic acid (66.4 mg, 1.11 mmol, 5 eq) and 2-methylpyridine borane (118.3 mg, 1.11 mmol, 5 eq), and the reaction mixture was stirred at 25° C. for 3 hours. Triethylamine was then added, and the resulting mixture (pH ˜8) was purified by flash chromatography on silica gel (gradient: 0˜5% CH₃OH in CH₂Cl₂) to afford tert-butyl 3-[7-(8-ethyl-3-hydroxy-1-naphthyl)-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]pyrido[4,3-d]pyrimidin-4-yl]-3,8-diazabicyclo[3.2.1]octane-8-carboxylate (160 mg, 38% yield) as a yellow solid. LC/MS (ESI) m/z: 586.7 [M/2+H]⁺.

Step 12: Preparation of (2S,4R)-1-[(2S)-2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methyl thiazol-5-yl)phenyl]ethyl]pyrrolidine-2-carboxamide

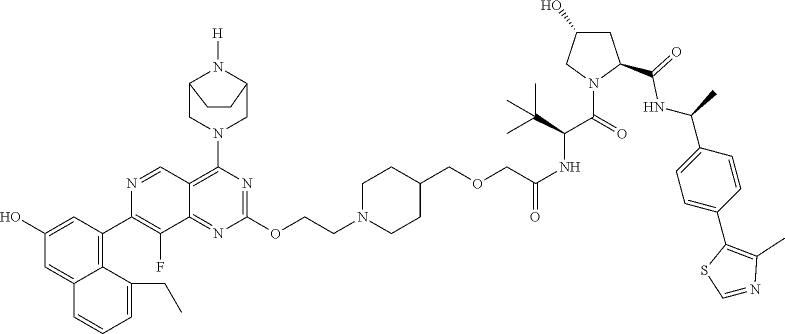 [SMILES: CCc1cccc2cc(O)cc(-c3ncc4c(N5CC6CCC(C5)N6)nc(OCCN5CCC(COCC(=O)N[C@H](C(=O)N6C[C@H](O)C[C@H]6C(=O)N[C@@H](C)c6ccc(-c7scnc7C)cc6)C(C)(C)C)CC5)nc4c3F)c12]

To a solution of tert-butyl 3-[7-(8-ethyl-3-hydroxy-1-naphthyl)-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1-piperidyl]ethoxy]pyrido[4,3-d]pyrimidin-4-yl]-3,8-diazabicyclo[3.2.1]octane-8-carboxylate (160 mg, 0.137 mmol, 1 eq) in DCE (2 mL) was added TFA (778.7 mg, 6.83 mmol, 0.51 mL, 50 eq), and the reaction mixture was stirred at 25° C. for 1 hour. The mixture was bubbled with N₂ to remove most of solvent, basified with saturated aqueous NaHCO₃ until pH=8, then extracted with CH₃OH/CH₂Cl₂ (3×30 mL, 1/10). The combined organic phase was washed with brine (20 mL), dried over anhydrous sodium sulfate, filtered, and concentrated. The resulting residue was purified by prep-HPLC (gradient 0-40% CH₃CN in water (0.225% formic acid). The pure fractions were combined and lyophilized to afford (2S,4R)-1-[(2S)-2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (48.0 mg, 30% yield, formic acid salt) as a white solid. LC/MS (ESI) m/z: 1071.9 [M+H]⁺. ¹H-NMR (400 MHz, CD₃OD) δ 9.10 (s, 1H), 8.89-8.84 (m, 1H), 8.49 (m, 1H, HCOOH), 7.65-7.60 (m, 1H), 7.45-7.33 (m, 5H), 7.29 (d, J=2.4 Hz, 1H), 7.16 (d, J=7.2 Hz, 1H), 7.01 (d, J=2.4 Hz, 1H), 5.03-4.95 (m, 1H), 4.82-4.66 (m, 7H), 4.60-4.51 (m, 1H), 4.45-4.36 (m, 1H), 4.08-3.72 (m, 7H), 3.65-3.55 (m, 2H), 3.50-3.38 (m, 4H), 3.01-2.84 (m, 2H), 2.47 (s, 3H), 2.40-2.18 (m, 3H), 2.10-1.87 (m, 8H), 1.66-1.54 (m, 2H), 1.47 (dd, J=7.2, 2.4 Hz, 2H), 1.08-0.96 (s, 9H), 0.89 (t, J=7.4 Hz, 3H).

Exemplary Synthesis of (2S,4R)-1-[(2S)-2-[3-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 8)

Step 1: Preparation of (2S,4R)-4-hydroxy-1-[(2S)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide

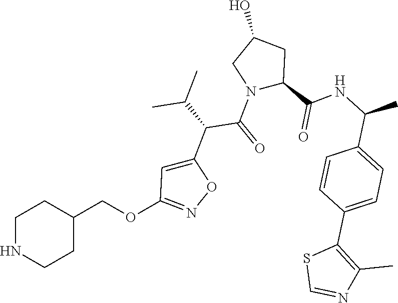 [SMILES: Cc1ncsc1-c1ccc([C@H](C)NC(=O)[C@@H]2C[C@@H](O)CN2C(=O)[C@H](c2cc(OCC3CCNCC3)no2)C(C)C)cc1]

To a solution of tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]piperidine-1-carboxylate (200 mg, 0.287 mmol, 1.0 eq) in CH₂Cl₂ (1 mL) was added HCl in dioxane (4 M, 1.0 mL, 14 eq), and the reaction mixture was stirred at 25° C. for 1 hour. The pH was adjusted to approximately 8 by progressively adding saturated aqueous Na₂CO₃. The resulting mixture was diluted with water (2 mL) and extracted with CH₂Cl₂/CH₃OH (3×20 mL, 10:1). The combined organic layers were washed brine (10 mL), dried over Na₂SO₄, filtered, and concentrated to afford (2S,4R)-4-hydroxy-1-[(2S)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (166 mg, 0.261 mmol, 91% yield) as a yellow gum. LC/MS (ESI) m/z: 596.4 [M+H]⁺.

Step 2: Preparation of tert-butyl 3-[7-(8-ethyl-3-hydroxy-1-naphthyl)-8-fluoro-2-[2-[4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate

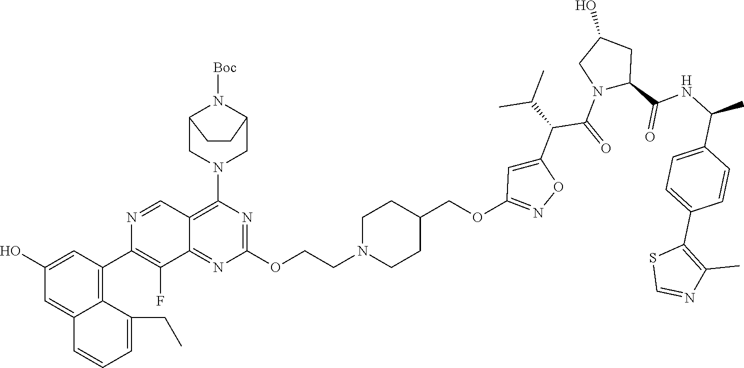 [SMILES: CCc1cccc2cc(O)cc(-c3ncc4c(N5CC6CCC(C5)N6C(=O)OC(C)(C)C)nc(OCCN5CCC(COc6cc([C@@H](C(=O)N7C[C@H](O)C[C@H]7C(=O)N[C@@H](C)c7ccc(-c8scnc8C)cc7)C(C)C)on6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (100 mg, 0.170 mmol, 1.0 eq) and (2S,4R)-4-hydroxy-1-[(2S)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (101 mg, 0.170 mmol, 1.0 eq) in IPA (1.0 mL) and CH₂Cl₂ (2.0 mL) was added acetic acid (681 mmol, 0.039 mL, 4 eq) and 2-methylpyridine borane (91 mg, 0.851 mmol, 5.0 eq), and the reaction mixture was stirred at 25° C. for 30 minutes. The pH was adjusted to approximately 8 by addition of triethylamine. The crude product was purified by flash chromatography on silica gel (gradient: 0˜8% CH₃OH in CH₂Cl₂) to afford tert-butyl 3-[7-(8-ethyl-3-hydroxy-1-naphthyl)-8-fluoro-2-[2-[4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate (133 mg, 0.102 mmol, 60% yield) as a yellow solid. LC/MS (ESI) m/z: 1167.5 [M+H]⁺.

Step 3: Preparation of (2S,4R)-1-[(2S)-2-[3-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

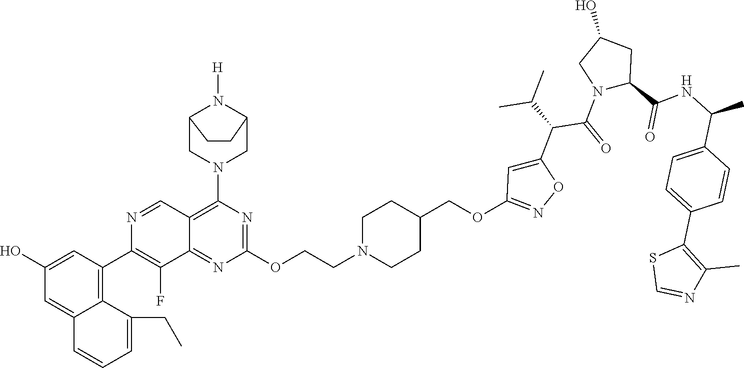 [SMILES: CCc1cccc2cc(O)cc(-c3ncc4c(N5CC6CCC(C5)N6)nc(OCCN5CCC(COc6cc([C@@H](C(=O)N7C[C@H](O)C[C@H]7C(=O)N[C@@H](C)c7ccc(-c8scnc8C)cc7)C(C)C)on6)CC5)nc4c3F)c12]

To a solution of tert-butyl 3-[7-(8-ethyl-3-hydroxy-1-naphthyl)-8-fluoro-2-[2-[4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate (130 mg, 0.111 mmol, 1.0 eq) in CH₂Cl₂ (3.0 mL) was added TFA (13.51 mmol, 1.0 mL, 121.28 eq), and the reaction mixture was stirred at 25° C. for 1 hour. The pH was adjusted to approximately 8 by addition of saturated aqueous NaHCO₃. Water (2.0 mL) was added, and the resulting mixture was extracted with CH₂Cl₂/CH₃OH (3×15 mL, 10:1). The combined organic layers were washed with brine (10 mL), dried over Na₂SO₄, filtered, and concentrated. The crude product was purified by prep-HPLC (gradient: 5-45% CH₃CN in water (0.225% formic acid)). The pure fractions were combined and concentrated under reduced pressure, then lyophilized to afford (2S,4R)-1-[(2S)-2-[3-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (52.5 mg, 0.048 mmol, 43% yield) as a white solid. LC/MS (ESI) m/z: 1067.4 [M+H]⁺. ¹H-NMR (400 MHz, CD₃OD) δ 9.08 (s, 1H), 8.90-8.85 (m, 1H), 8.49 (s, 1H), 7.63 (d, J=8.2 Hz, 1H), 7.48-7.33 (m, 5H), 7.29 (d, J=1.6 Hz, 1H), 7.16 (d, J=7.2 Hz, 1H), 7.00 (t, J=2.4 Hz, 1H), 6.04-5.95 (m, 1H), 5.01-4.95 (m, 1H), 4.78-4.72 (m, 3H), 4.70-4.66 (m, 1H), 4.57 (t, J=8.2 Hz, 1H), 4.42 (br s, 1H), 4.10-4.01 (m, 2H), 3.94-3.88 (m, 2H), 3.86-3.74 (m, 3H), 3.74-3.68 (m, 1H), 3.67-3.62 (m, 1H), 3.42 (br d, J=10.6 Hz, 2H), 3.21 (br d, J=4.8 Hz, 2H), 2.63 (br t, J=11.4 Hz, 2H), 2.49-2.45 (m, 3H), 2.39-2.19 (m, 4H), 2.02-1.87 (m, 8H), 1.60-1.51 (m, 2H), 1.48 (d, J=7.0 Hz, 3H), 1.08-0.93 (m, 3H), 0.93-0.82 (m, 6H).

Exemplary Synthesis of (2S,4R)-1-[(2R)-2-[3-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 20)

Step 1: Preparation of tert-butyl 3-[7-(8-ethyl-3-hydroxy-1-naphthyl)-8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate

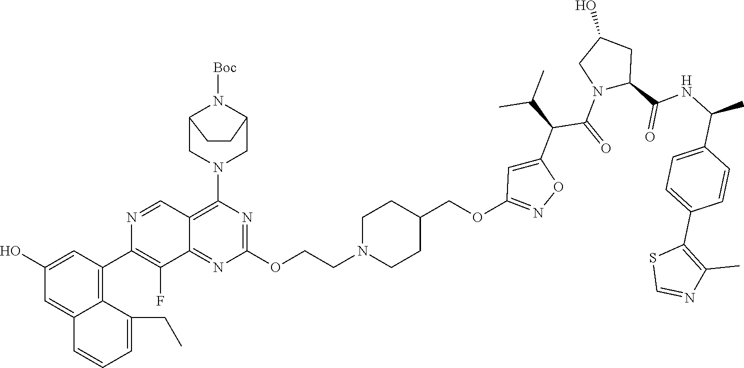 [SMILES: CCc1cccc2cc(O)cc(-c3ncc4c(N5CC6CCC(C5)N6C(=O)OC(C)(C)C)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (130 mg, 0.221 mmol, 1 eq) and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (131.8 mg, 0.221 mmol, 1 eq) in CH₂Cl₂ (2 mL) and IPA (2 mL) were added acetic acid (66.4 mg, 1.11 mmol, 5 eq) and 2-methylpyridine borane (118.3 mg, 1.11 mmol, 5 eq), and the reaction mixture was stirred at 25° C. for 2 hours. The pH was adjusted to 8 by addition of triethylamine, and the resulting mixture was purified by flash chromatography on silica gel (gradient: 0˜5% CH₃OH in CH₂Cl₂) to afford tert-butyl 3-[7-(8-ethyl-3-hydroxy-1-naphthyl)-8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate (210 mg, 54%) as a yellow solid. LC/MS (ESI) m/z: 584.6 [M/2+H]⁺.

Step 2: Preparation of (2S,4R)-1-[(2R)-2-[3-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

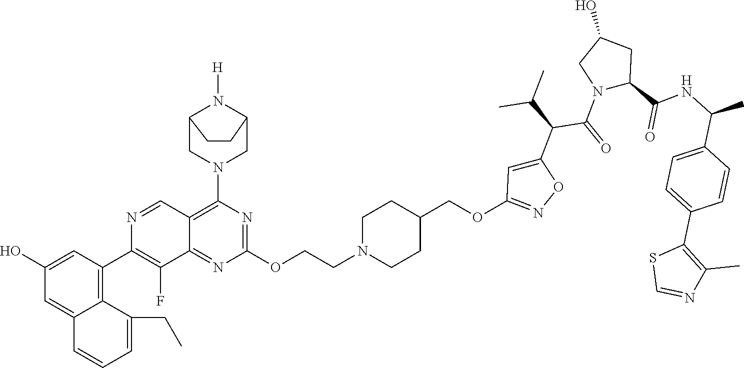 [SMILES: CCc1cccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of tert-butyl 3-[7-(8-ethyl-3-hydroxy-1-naphthyl)-8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate (210 mg, 0.180 mmol, 1 eq) in DCE (2.7 mL) was added TFA (1.03 g, 8.99 mmol, 50 eq), and the reaction mixture was stirred at 25° C. for 1 hour. The mixture was bubbled with N₂ to remove most of solvent, basified with aqueous saturated aqueous NaHCO₃ until pH reached 8, and then filtered. The resulting material was purified by prep-HPLC (30-80% CH₃CN in water (NH₄OH)). The pure fractions were combined and lyophilized to afford (2S,4R)-1-[(2R)-2-[3-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (42.6 mg, 22% yield) as a white solid. LC/MS (ESI) m/z: 1067.8 [M+H]⁺. ¹H-NMR (400 MHz, CD₃OD) δ 9.05 (s, 1H), 8.87 (s, 1H), 7.63 (d, J=8.4 Hz, 1H), 7.47-7.33 (m, 5H), 7.29 (d, J=2.4 Hz, 1H), 7.16 (d, J=6.8 Hz, 1H), 7.01 (d, J=2.4 Hz, 1H), 5.97 (s, 1H), 5.08-4.97 (m, 2H), 4.70-4.61 (m, 4H), 4.53-4.48 (m, 1H), 4.46-4.41 (m, 1H), 4.08-4.00 (m, 2H), 3.86-3.59 (m, 7H), 3.20-3.11 (m, 2H), 2.97-2.88 (m, 2H), 2.48 (s, 3H), 2.41-2.13 (m, 6H), 2.00-1.77 (m, 8H), 1.61-1.49 (m, 2H), 1.48-1.35 (m, 2H), 1.05 (d, J=6.4 Hz, 3H), 0.94-0.85 (m, 6H).

Exemplary Synthesis of (2S,4R)-1-[(2S)-2-[[2-[[1-[2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 19)

Step 1: Preparation of 3-oxabicyclo[3.2.1]octane-2,4-dione

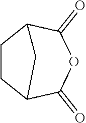

A mixture of cyclopentane-1,3-dicarboxylic acid (5.90 g, 37.3 mmol, 1 eq) in Ac₂O (30 mL) was stirred at 140° C. for 24 hours. The reaction mixture was concentrated under reduced pressure, and the resulting was washed with EtOAc/petroleum ether (3×30 mL, 1/30) to give 3-oxabicyclo[3.2.1]octane-2,4-dione (4.70 g, 33.5 mmol, 90% yield) as a gray solid. ¹H-NMR (400 MHz, DMSO-d₆) δ 3.23-3.11 (m, 2H), 2.35 (d, J=12.4 Hz, 1H), 2.15-2.05 (m, 2H), 1.94-1.84 (m, 2H), 1.71-1.64 (td, J=12.8, 4.0 Hz, 1H).

Step 2: Preparation of 3-azabicyclo[3.2.1]octane-2,4-dione

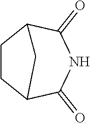

A mixture of 3-oxabicyclo[3.2.1]octane-2,4-dione (2.70 g, 19.3 mmol, 1 eq) in NH₃/MeOH (7 M, 27.5 mL, 10 eq) was stirred at 25° C. for 0.5 hour. The reaction mixture was concentrated under reduced pressure, and the resulting residue was heated at 180° C. for 15 minutes. After cooling to 25° C., the residue was dissolved in CH₂Cl₂ (50 mL), and the resulting mixture was filtered through silica gel. The filtrate was concentrated under reduced pressure to give 3-azabicyclo[3.2.1]octane-2,4-dione (755 mg, 5.43 mmol, 28% yield) as a white solid. LC/MS (ESI) m/z: 140.1 [M+H]⁺. ¹H-NMR (400 MHz, DMSO-d₆) δ 10.43 (brs, 1H), 2.88-2.82 (m, 2H), 2.10 (d, J=12.0 Hz, 1H), 2.06-1.98 (m, 2H), 1.77-1.69 (m, 2H), 1.59 (dt, J=12.0, 4.0 Hz, 1H).

Step 3: Preparation of 3-azabicyclo[3.2.1]octane

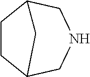

To a solution of 3-azabicyclo[3.2.1]octane-2,4-dione (755 mg, 5.43 mmol, 1 eq) in THF (20 mL) at 0° C. was added LiAlH₄ (412 mg, 10.9 mmol, 2 eq), and the reaction mixture was stirred at 66° C. under N₂ for 3 hours. The reaction mixture was quenched sequentially by addition of water (0.5 mL), 15% aq. NaOH (0.5 mL), and water (1.5 mL), then dried over anhydrous Na₂SO₄, filtered, and concentrated to give 3-azabicyclo[3.2.1]octane (332 mg, crude) as a colorless oil.

Step 4: Preparation of 4-(3-azabicyclo[3.2.1]octan-3-yl)-2,7-dichloro-8-fluoro-pyrido[4,3-d]pyrimidine

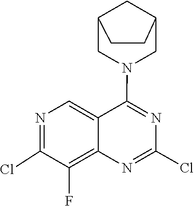 [SMILES: Fc1c(Cl)ncc2c(N3CC4CCC(C4)C3)nc(Cl)nc12]

To a solution of 2,4,7-trichloro-8-fluoro-pyrido[4,3-d]pyrimidine (750 mg, 2.97 mmol, 1 eq) in CH₂Cl₂ (20 mL) at −40° C. were added DIEA (768 mg, 5.94 mmol, 1.03 mL, 2 eq) and 3-azabicyclo[3.2.1]octane (330 mg, 2.97 mmol, 1 eq), and the reaction mixture was stirred at −40° C. under N₂ for 0.5 hour. The reaction mixture was poured onto water (20 mL) and extracted with CH₂Cl₂ (2×20 mL). The combined organic extract was washed with brine (2×20 mL), dried over anhydrous Na₂SO₄, filtered, and concentrated. The resulting crude product was purified by flash silica gel chromatography (gradient: 0˜10% THF/petroleum ether) to give 4-(3-azabicyclo[3.2.1]octan-3-yl)-2,7-dichloro-8-fluoro-pyrido[4,3-d]pyrimidine (348 mg, 1.06 mmol, 36% yield) as a yellow solid. LC/MS (ESI) m/z: 327.0 [M+H]⁺.

Step 5: Preparation of 4-(3-azabicyclo[3.2.1]octan-3-yl)-7-chloro-2-(2,2-dimethoxyethoxy)-8-fluoro-pyrido[4,3-d]pyrimidine

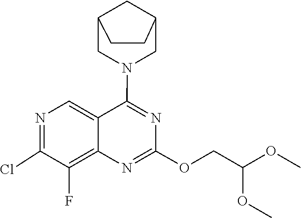 [SMILES: COC(COc1nc(N2CC3CCC(C3)C2)c2cnc(Cl)c(F)c2n1)OC]

To a solution of 4-(3-azabicyclo[3.2.1]octan-3-yl)-2,7-dichloro-8-fluoro-pyrido[4,3-d]pyrimidine (1.48 g, 4.52 mmol, 1 eq) and 2,2-dimethoxyethanol (720 mg, 6.79 mmol, 1.5 eq) in CH₃CN (5 mL) and THF (25 mL) were added DABCO (50.7 mg, 0.452 mmol, 0.1 eq) and Cs₂CO₃ (1.77 g, 5.43 mmol, 1.2 eq), and the reaction mixture was stirred at 25° C. for 16 hours. The mixture was diluted with CH₂Cl₂ (50 mL), then filtered, and the filtrate was evaporated. The resulting material was purified by flash chromatography on silica gel (gradient: 5˜11% EtOAc in petroleum ether) to afford 4-(3-azabicyclo[3.2.1]octan-3-yl)-7-chloro-2-(2,2-dimethoxyethoxy)-8-fluoro-pyrido[4,3-d]pyrimidine (1.29 g, 2.96 mmol, 65% yield) as a yellow solid. LC/MS (ESI) m/z: 397.2 [M+H]⁺.

Step 6: Preparation of 4-(3-azabicyclo[3.2.1]octan-3-yl)-2-(2,2-dimethoxyethoxy)-7-[8-ethyl-3-(methoxymethoxy)-1-naphthyl]-8-fluoro-pyrido[4,3-d]pyrimidine

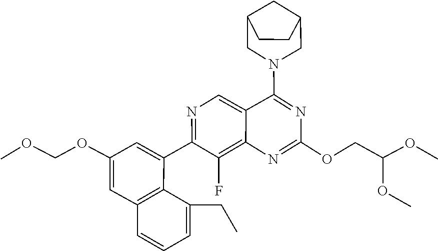

To a solution of 4-(3-azabicyclo[3.2.1]octan-3-yl)-7-chloro-2-(2,2-dimethoxyethoxy)-8-fluoro-pyrido[4,3-d]pyrimidine (300 mg, 0.756 mmol, 1.0 eq) and 2-[8-ethyl-3-(methoxymethoxy)-1-naphthyl]-4,4,5,5-tetramethyl-1,3,2-dioxaborolane (310 mg, 0.907 mmol, 1.2 eq) in dioxane (4 mL) and H₂O (0.8 mL) were added Cs₂CO₃ (616 mg, 1.89 mmol, 2.5 eq) and [1,1′-bis(di-tert-butylphosphino)ferrocene]dichloropalladium (II) (98.5 mg, 0.151 mmol, 0.2 eq) under N₂, and the reaction mixture was stirred at 105° C. for 16 hours under N₂. The reaction mixture was diluted with CH₂Cl₂ (40 mL), dried over anhydrous sodium sulfate, and concentrated. The resulting residue was purified by flash chromatography on silica gel (gradient: 0˜17% EtOAc in petroleum ether) to afford 4-(3-azabicyclo[3.2.1]octan-3-yl)-2-(2,2-dimethoxyethoxy)-7-[8-ethyl-3-(methoxymethoxy)-1-naphthyl]-8-fluoro-pyrido[4,3-d]pyrimidine (125 mg, 0.211 mmol, 28% yield) as a yellow solid. LC/MS (ESI) m/z: 577.3 [M+H]⁺.

Step 7: Preparation of 2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyacetaldehyde

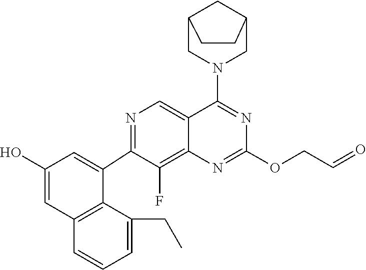 [SMILES: CCc1cccc2cc(O)cc(-c3ncc4c(N5CC6CCC(C6)C5)nc(OCC=O)nc4c3F)c12]

To a solution of 4-(3-azabicyclo[3.2.1]octan-3-yl)-2-(2,2-dimethoxyethoxy)-7-[8-ethyl-3-(methoxymethoxy)-1-naphthyl]-8-fluoro-pyrido[4,3-d]pyrimidine (120 mg, 0.208 mmol, 1.0 eq) in acetone (0.5 mL) was added HCl (520 uL, 12 M, 30 eq) dropwise, and the reaction mixture was stirred at 20° C. for 5 minutes. A fine precipitate was formed. The pH of the mixture was adjusted to pH 7˜8 by addition of saturated aqueous NaHCO₃, and the resulting aqueous mixture was extracted with CH₂Cl₂/i-PrOH (4×30 mL, 5/1). The combined organic extract was washed with brine (10 mL), dried over anhydrous sodium sulfate, filtered, and concentrated to afford 2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyacetaldehyde (101 mg, 0.154 mmol, 74% yield) as a yellow solid. LC/MS (ESI) m/z: 487.2 [M+H]⁺.

Step 8: Preparation of (2S,4R)-1-[(2S)-2-[[2-[[1-[2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

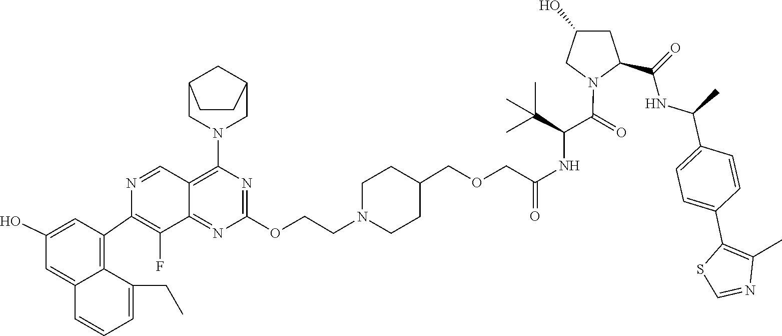 [SMILES: CCc1cccc2cc(O)cc(-c3ncc4c(N5CC6CCC(C6)C5)nc(OCCN5CCC(COCC(=O)N[C@H](C(=O)N6C[C@H](O)C[C@H]6C(=O)N[C@@H](C)c6ccc(-c7scnc7C)cc6)C(C)(C)C)CC5)nc4c3F)c12]

To a solution of 2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyacetaldehyde (100 mg, 0.206 mmol, 1.0 eq) and (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (123 mg, 0.206 mmol, 1.0 eq) in CH₂Cl₂ (1 mL) and IPA (1 mL) was added acetic acid (47 uL, 0.822 mmol, 4.0 eq) and 2-methylpyridine borane (110 mg, 1.03 mmol, 5.0 eq), and the reaction mixture was stirred at 20° C. for 3 hours. The reaction mixture was concentrated, and the resulting was purified by prep-HPLC (gradient: 10-60% CH₃CN in water (0.225% formic acid)). The pure fractions were combined and lyophilized to afford (2S,4R)-1-[(2S)-2-[[2-[[1-[2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (32.8 mg, 0.0305 mmol, 15% yield) as a white solid. LC/MS (ESI) m/z: 535.9 [M+H]⁺. ¹H-NMR (400 MHz, CD₃OD) δ 9.05 (s, 1H), 8.87 (s, 1H), 7.63-7.60 (m, 1H), 7.43-7.33 (m, 5H), 7.29-7.24 (m, 1H), 7.17-7.13 (m, 1H), 7.03-7.01 (m, 1H), 5.06-4.91 (m, 2H), 4.80-4.65 (m, 6H), 4.64-4.47 (m, 3H), 4.45-4.33 (m, 1H), 4.04-3.92 (m, 2H), 3.87-3.69 (m, 2H), 3.69-3.53 (m, 3H), 3.45-3.33 (m, 3H), 3.20-3.01 (m, 2H), 2.46 (s, 5H), 2.35-2.18 (m, 2H), 2.00-1.83 (m, 3H), 1.80-1.65 (m, 4H), 1.64-1.53 (m, 3H), 1.50-1.40 (m, 4H), 1.04-1.00 (m, 9H), 0.91-0.84 (m, 3H).

Exemplary Synthesis of (2S,4R)-1-[(2S)-2-[[2-[[1-[2-[4-(3-azabicyclo[3.2.1]octan-3-yl)-7-(8-ethynyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 17)

Step 1: Preparation of [3-(methoxymethoxy)-8-(2-triisopropylsilylethynyl)-1-naphthyl]trifluoromethanesulfonate

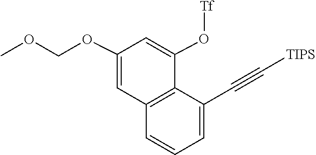

To a solution of 3-(methoxymethoxy)-8-(2-triisopropylsilylethynyl) naphthalen-1-ol (3.0 g, 7.80 mmol, 1 eq) in CH₂Cl₂ (30 mL) at −40° C. were added DIEA (3.02 g, 23.40 mmol, 3 eq) and Tf₂O (3.30 g, 11.70 mmol, 1.5 eq), and the reaction mixture was stirred at −40° C. for 30 minutes under N₂. The reaction mixture was quenched by addition of water (20 mL) at −40° C., and then extracted with CH₂Cl₂ (3×50 mL). The combined organic extract was washed with brine (40 mL), dried over anhydrous sodium sulfate, filtered, and concentrated. The resulting residue was purified by flash chromatography on silica gel (gradient: 0˜1% EtOAc in petroleum ether) to afford [3-(methoxymethoxy)-8-(2-triisopropylsilylethynyl)-1-naphthyl]trifluoromethanesulfonate (3.9 g, 7.55 mmol, 97% yield) as a yellow oil.

Step 2: Preparation of triisopropyl-[2-[6-(methoxymethoxy)-8-(4,4,5,5-tetramethyl-1,3,2-dioxaborolan-2-yl)-1-naphthyl]ethynyl]silane

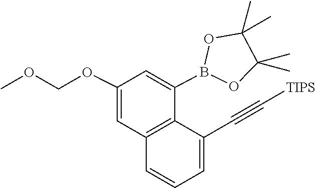

A mixture of [3-(methoxymethoxy)-8-(2-triisopropylsilylethynyl)-1-naphthyl]trifluoromethanesulfonate (3.9 g, 7.55 mmol, 1 eq), 4,4,5,5-tetramethyl-2-(4,4,5,5-tetramethyl-1,3,2-dioxaborolan-2-yl)-1,3,2-dioxaborolane (4.22 g, 16.61 mmol, 2.2 eq), Pd(dppf) Cl₂ (1.10 g, 1.51 mmol, 0.2 eq), and KOAc (2.59 g, 26.42 mmol, 3.5 eq) in toluene (55 mL) was degassed and purged with N₂ (3×), and the mixture was stirred at 110° C. for 48 hours under N₂ atmosphere. The reaction mixture was filtered, and the filter cake was washed with MTBE (3×40 mL). The filtrate was concentrated, and the resulting residue was purified by flash chromatography on silica gel (gradient: 0˜3% EtOAc in petroleum ether) to afford triisopropyl-[2-[6-(methoxymethoxy)-8-(4,4,5,5-tetramethyl-1,3,2-dioxaborolan-2-yl)-1-naphthyl]ethynyl]silane (2.41 g, 4.63 mmol, 61% yield) as a yellow solid. ¹H-NMR (400 MHz, CDCl₃) δ 7.73-7.66 (m, 2H), 7.48 (d, J=2.4 Hz, 1H), 7.42-7.35 (m, 1H), 7.35-7.31 (m, 1H), 5.29 (s, 2H), 3.51 (s, 3H), 1.44 (s, 12H), 1.19-1.14 (m, 21H).

Step 3: Preparation of 2-[8-[[4-(3-azabicyclo[3.2.1]octan-3-yl)-2-(2,2-dimethoxyethoxy)-8-fluoro-pyrido[4,3-d]pyrimidin-7-yl]-6-(methoxymethoxy)-1-naphthyl]ethynyl-triisopropyl-silane

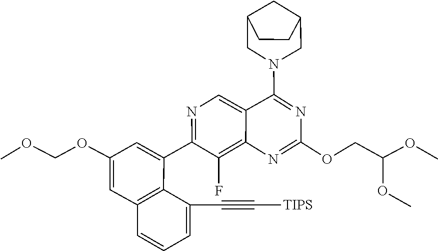

To a solution of 4-(3-azabicyclo[3.2.1]octan-3-yl)-7-chloro-2-(2,2-dimethoxyethoxy)-8-fluoro-pyrido[4,3-d]pyrimidine (220 mg, 0.554 mmol, 1 eq) and triisopropyl-[2-[6-(methoxymethoxy)-8-(4,4,5,5-tetramethyl-1,3,2-dioxaborolan-2-yl)-1-naphthyl]ethynyl]silane (329.0 mg, 0.665 mmol, 1.2 eq) in dioxane (4 mL) and H₂O (0.8 mL) were added Cs₂CO₃ (451 mg, 1.39 mmol, 2.5 eq) and [1,1′-bis(di-tert-butylphosphino)ferrocene]dichloropalladium (II) (72 mg, 0.111 mmol, 0.2 eq), and the reaction mixture was stirred at 105° C. for 16 hours under N₂. CH₂Cl₂ (90 mL) was then added, and the resulting mixture was dried over anhydrous sodium sulfate, filtered, and concentrated. The resulting residue was purified by flash chromatography on silica gel (gradient: 10˜23% EtOAc to afford in petroleum ether) 2-[8-[[4-(3-azabicyclo[3.2.1]octan-3-yl)-2-(2,2-dimethoxyethoxy)-8-fluoro-pyrido[4,3-d]pyrimidin-7-yl]-6-(methoxymethoxy)-1-naphthyl]ethynyl-triisopropyl-silane (183 mg, 45% yield) as a yellow oil. LC/MS (ESI) m/z: 729.4 [M+H]⁺.

Step 4: Preparation of 2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyacetaldehyde

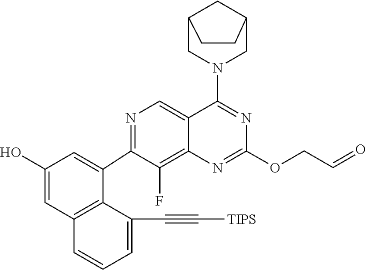

To a solution of 2-[8-[[4-(3-azabicyclo[3.2.1]octan-3-yl)-2-(2,2-dimethoxyethoxy)-8-fluoro-pyrido[4,3-d]pyrimidin-7-yl]-6-(methoxymethoxy)-1-naphthyl]ethynyl-triisopropyl-silane (130 mg, 0.178 mmol, 1 eq) in acetone (0.45 mL) was added HCl (12 M, 0.45 mL, 30 eq), and the reaction mixture was stirred at 20° C. for 6 minutes. The pH of the reaction mixture was basified to pH=8 by addition of saturated aqueous NaHCO₃, and the resulting aqueous mixture was extracted with EtOAc/THF (3×20 mL, 5/1). The combined organic extract was washed with brine (20 mL), dried over anhydrous sodium sulfate, filtered, and concentrated to give 2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyacetaldehyde (158 mg, crude) as a yellow solid. LC/MS (ESI) m/z: 639.3 [M+H]⁺.

Step 5: Preparation of (2S,4R)-1-[(2S)-2-[[2-[[1-[2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

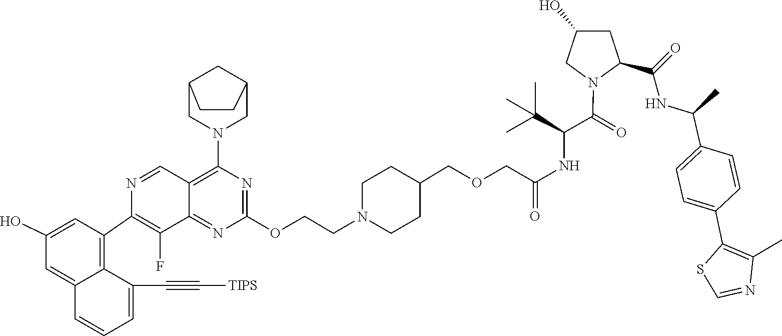

To a solution of 2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyacetaldehyde (150 mg, 0.235 mmol, 1 eq) and (2S,4R)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide (141 mg, 0.235 mmol, 1 eq) in CH₂Cl₂ (2 mL) and IPA (2 mL) was added acetic acid (70 mg, 1.17 mmol, 5 eq) and 2-methylpyridine borane (125 mg, 1.17 mmol, 5 eq), and the reaction mixture was stirred at 25° C. for 2 hours. Triethylamine was then added to adjust the pH to ˜8, and the resulting mixture was purified by flash chromatography on silica gel (gradient: 0˜10% CH₃OH in CH₂Cl₂) followed by prep-HPLC (gradient: 20-70% CH₃CN in water (0.225% formic acid)). The pure fractions were combined and lyophilized to afford (2S,4R)-1-[(2S)-2-[[2-[[1-[2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (85 mg, 27% yield, formic acid salt) as a white solid. LC/MS (ESI) m/z: 1223.5 [M+H]⁺.

Step 6: Preparation of (2S,4R)-1-[(2S)-2-[[2-[[1-[2-[4-(3-azabicyclo[3.2.1]octan-3-yl)-7-(8-ethynyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

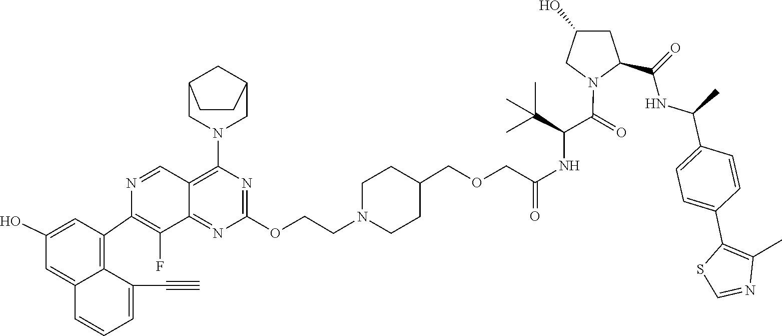 [SMILES: C#Cc1cccc2cc(O)cc(-c3ncc4c(N5CC6CCC(C6)C5)nc(OCCN5CCC(COCC(=O)N[C@H](C(=O)N6C[C@H](O)C[C@H]6C(=O)N[C@@H](C)c6ccc(-c7scnc7C)cc6)C(C)(C)C)CC5)nc4c3F)c12]

To a solution of (2S,4R)-1-[(2S)-2-[[2-[[1-[2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (85 mg, 0.0695 mmol, 1 eq) in DMF (1.5 mL) was added CsF (106 mg, 0.695 mmol, 10 eq), and the reaction mixture was stirred at 25° C. for 16 hours. The mixture was filtered and purified by prep-HPLC (gradient: 30-90% CH₃CN in water (NH₄HCO₃)). The pure fractions were combined and lyophilized to afford (2S,4R)-1-[(2S)-2-[[2-[[1-[2-[4-(3-azabicyclo[3.2.1]octan-3-yl)-7-(8-ethynyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (31.5 mg, 41% yield) as a yellow solid. LC/MS (ESI) m/z: 1066.8 [M+H]⁺. ¹H-NMR (400 MHz, CD₃OD) δ 8.99 (s, 1H), 8.87 (s, 1H), 7.84-7.77 (m, 1H), 7.52-7.47 (m, 1H), 7.45-7.35 (m, 5H), 7.33-7.29 (m, 1H), 7.16 (d, J=2.4 Hz, 1H), 5.02-4.96 (m, 1H), 4.67-4.52 (m, 7H), 4.04-3.91 (m, 2H), 3.86-3.56 (m, 4H), 3.42 (d, J=6.4 Hz, 2H), 3.19-3.07 (m, 2H), 3.01 (s, 1H), 2.92-2.84 (m, 2H), 2.47 (s, 3H), 2.45-2.39 (m, 2H), 2.30-2.14 (m, 3H), 2.03-1.89 (m, 2H), 1.87-1.68 (m, 6H), 1.66-1.55 (m, 2H), 1.48 (d, J=7.2 Hz, 3H), 1.42-1.27 (m, 2H), 1.09-0.98 (s, 9H).

Exemplary Synthesis of (2S,4R)-1-[(2R)-2-[3-[[1-[2-[4-(3-azabicyclo[3.2.1]octan-3-yl)-7-(8-ethynyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 18)

Step 1: Preparation of (2S,4R)-1-[(2R)-2-[3-[[1-[2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

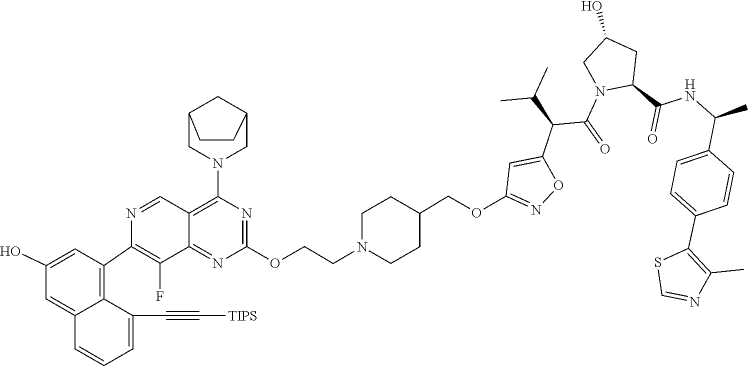

To a solution of 2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyacetaldehyde (160 mg, 0.250 mmol, 1 eq) and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (149 mg, 0.250 mmol, 1 eq) in CH₂Cl₂ (2.5 mL) and IPA (2.5 mL) were added acetic acid (75 mg, 1.25 mmol, 5 eq) and 2-methylpyridine borane (134 mg, 1.25 mmol, 5 eq), and the reaction mixture was stirred at 25° C. for 2 hours. Triethylamine was then added to adjust the pH to ˜8, and the resulting mixture was purified by flash chromatography on silica gel (gradient: 0˜8% CH₃OH in CH₂Cl₂) to afford (2S,4R)-1-[(2R)-2-[3-[[1-[2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (265 mg, 76% yield) as a yellow solid. LC/MS (ESI) m/z: 610.0 [M/2+H]⁺.

Step 2: Preparation of (2S,4R)-1-[(2R)-2-[3-[[1-[2-[4-(3-azabicyclo[3.2.1]octan-3-yl)-7-(8-ethynyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

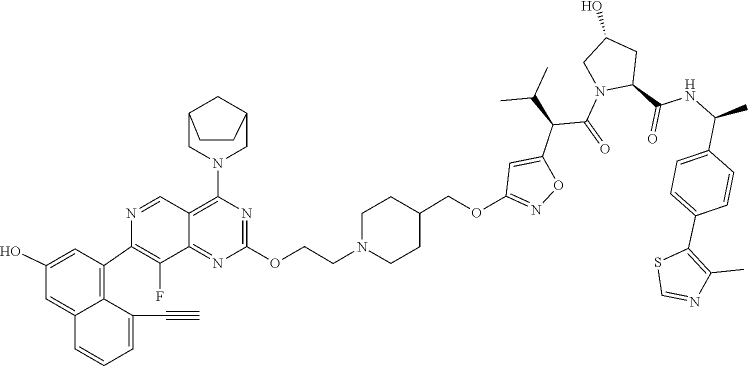 [SMILES: C#Cc1cccc2cc(O)cc(-c3ncc4c(N5CC6CCC(C6)C5)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of (2S,4R)-1-[(2R)-2-[3-[[1-[2-[4-(3-azabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (140 mg, 0.115 mmol, 1 eq) in DMF (2 mL) was added CsF (262 mg, 1.72 mmol, 15 eq), and the reaction mixture was stirred at 25° C. for 16 hours. The mixture was filtered, and then purified by prep-HPLC (10-60% CH₃CN in water (0.225% formic acid)) followed by prep-HPLC (30-90% CH₃CN in water (NH₄HCO₃)). The pure fractions were combined and lyophilized to afford (2S,4R)-1-[(2R)-2-[3-[[1-[2-[4-(3-azabicyclo[3.2.1]octan-3-yl)-7-(8-ethynyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (18.4 mg, 15% yield) as a yellow solid. LC/MS (ESI) m/z: 1062.8 [M+H]⁺. 1H-NMR (400 MHz, CD₃OD) δ 8.99 (s, 1H), 8.91-8.83 (m, 1H), 7.81 (d, J=8.4 Hz, 1H), 7.53-7.48 (m, 1H), 7.47-7.35 (m, 5H), 7.32 (d, J=2.4 Hz, 1H), 7.16 (d, J=2.4 Hz, 1H), 6.04-5.94 (m, 1H), 5.07-4.99 (m, 1H), 4.75-4.39 (m, 8H), 4.09-3.99 (m, 2H), 3.88-3.45 (m, 5H), 3.19-3.06 (m, 2H), 3.01 (s, 1H), 2.88 (t, J=5.6 Hz, 2H), 2.49 (s, 3H), 2.45-2.41 (m, 2H), 2.40-2.13 (m, 4H), 2.00-1.89 (m, 2H), 1.87-1.69 (m, 6H), 1.66-1.56 (m, 2H), 1.52 (d, J=6.8 Hz, 2H), 1.45-1.28 (m, 2H), 1.05 (d, J=6.4 Hz, 3H), 0.94-0.85 (m, 3H).

Exemplary Synthesis of (2S,4R)-1-[(2S)-2-[[2-[[1-[2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3-methyl-butanoyl]-4-hydroxy-N-[(1S)-1-[4-(4-methylthiazol-5-yl)phenyl]ethyl]pyrrolidine-2-carboxamide (Compound 14)

Step 1: Preparation of (2S,4R)-1-[(2S)-2-[[2-[[1-[2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3-methyl-butanoyl]-4-hydroxy-N-[(1S)-1-[4-(4-methylthiazol-5-yl)phenyl]ethyl]pyrrolidine-2-carboxamide

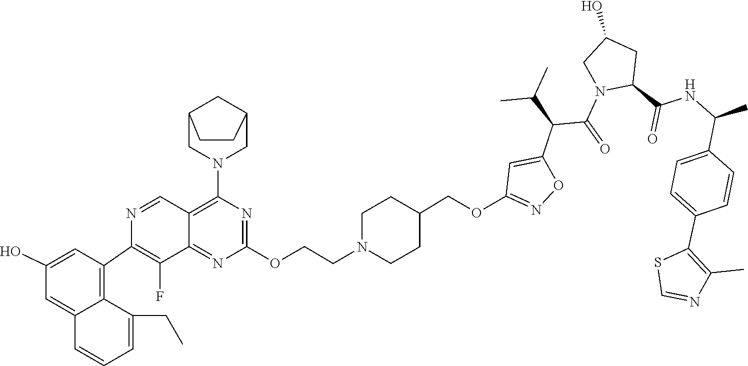 [SMILES: CCc1cccc2cc(O)cc(-c3ncc4c(N5CC6CCC(C6)C5)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of 2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyacetaldehyde (96 mg, 0.197 mmol, 1.0 eq) and (2S,4R)-4-hydroxy-1-[(2S)-3-methyl-2-[[2-(4-piperidylmethoxy)acetyl]amino]butanoyl]-N-[(1S)-1-[4-(4-methylthiazol-5-yl)phenyl]ethyl]pyrrolidine-2-carboxamide (116 mg, 0.197 mmol, 1.0 eq) in IPA (1 mL) and CH₂Cl₂ (1 mL) were added acetic acid (0.045 mL, 0.789 mmol, 4.0 eq) and 2-methylpyridine borane (106 mg, 0.986 mmol, 5.0 eq), and the reaction mixture was stirred at 25° C. for 12 hours. The reaction mixtures was concentrated, and the resulting residue was purified by pre-HPLC (30-90% CH₃CN in water (NH₄OH)). The pure fractions were combined and lyophilized to afford (2S,4R)-1-[(2S)-2-[[2-[[1-[2-[4-(3-azabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3-methyl-butanoyl]-4-hydroxy-N-[(1S)-1-[4-(4-methylthiazol-5-yl)phenyl]ethyl]pyrrolidine-2-carboxamide (25.4 mg, 0.023 mmol, 12% yield) as a white solid. LC/MS (ESI) m/z: 1066.8 [M+H]⁺. ¹H-NMR (400 MHz, CD₃OD) δ 9.03 (s, 1H), 8.87 (s, 1H), 7.62 (d, J=8.0 Hz, 1H), 7.46-7.37 (m, 4H), 7.37-7.32 (m, 1H), 7.28 (d, J=2.4 Hz, 1H), 7.15 (d, J=7.2 Hz, 1H), 7.02 (d, J=2.4 Hz, 1H), 6.02-5.89 (m, 1H), 5.03 (d, J=6.8 Hz, 1H), 4.68-4.60 (m, 7H), 4.42 (s, 1H), 4.04 (d, J=6.0 Hz, 2H), 3.87-3.78 (m, 1H), 3.70-3.56 (m, 4H), 3.13 (d, J=11.2 Hz, 2H), 2.92-2.84 (m, 2H), 2.50-2.45 (m, 3H), 2.43 (s, 2H), 2.38-2.13 (m, 6H), 1.98-1.88 (m, 2H), 1.81 (d, J=12.0 Hz, 2H), 1.76-1.70 (m, 2H), 1.62-1.48 (m, 5H), 1.47-1.36 (m, 2H), 1.05 (d, J=6.4 Hz, 3H), 0.93-0.86 (m, 6H).

Exemplary Synthesis of (2S,4R)-1-[(2S)-2-[[2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 13)

Step 1: Preparation of tert-butyl 4-[[2-(2-ethoxy-2-oxo-ethoxy)ethyl]piperidine-1-carboxylate

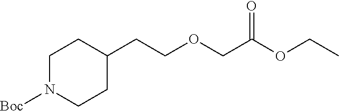 [SMILES: CCOC(=O)COCCC1CCN(C(=O)OC(C)(C)C)CC1]

To a solution of tert-butyl 4-(2-hydroxyethyl) piperidine-1-carboxylate (5.0 g, 21.80 mmol, 1 eq) in CH₂Cl₂ (100 mL) were added Rh₂(OAc)₄ (96 mg, 218.04 umol, 0.01 eq) and ethyl 2-diazoacetate (5.0 g, 43.61 mmol, 4.6 mL, 2.0 eq) dropwise, and the reaction mixture was stirred at 25° C. for 15 hours. The mixture was washed with brine (3×50 mL), and the organic layer was dried over Na₂SO₄, filtered, and concentrated. The resulting residue was purified by flash chromatography on silica gel (gradient: 0˜8% THF in petroleum ether) to afford tert-butyl 4-[[2-(2-ethoxy-2-oxo-ethoxy)ethyl]piperidine-1-carboxylate (5.0 g, 15.85 mmol, 73% yield) as a light yellow oil. ¹H-NMR (400 MHz, CDCl₃) δ 4.23 (q, J=2.4 Hz, 2H), 4.09 (s, 1H), 4.06 (s, 3H), 3.58 (t, J=6.4 Hz, 2H), 2.70 (t, J=4.0 Hz, 2H), 1.69 (d, J=3.2 Hz, 2H), 1.63-1.55 (m, 3H), 1.45 (s, 9H), 1.29 (t, J=2.0 Hz, 3H), 1.16-1.06 (m, 2H).

Step 2: Preparation of 2-[2-(1-tert-butoxycarbonyl-4-piperidyl)ethoxy]acetic acid

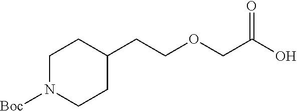 [SMILES: CC(C)(C)OC(=O)N1CCC(CCOCC(=O)O)CC1]

To a solution of tert-butyl 4-[[2-(2-ethoxy-2-oxo-ethoxy)ethyl]piperidine-1-carboxylate (5.0 g, 15.85 mmol, 1.0 eq) in THF (30 mL) and H₂O (20 mL) was added LiOH·H₂O (2.0 g, 47.56 mmol, 3.0 eq), and the reaction mixture was stirred at 25° C. for 2 hours. The pH of the mixture was adjusted to pH ˜3 by addition of 2N HCl. The organic layer was washed with brine (3×30 mL), dried over Na₂SO₄, filtered, and concentrated to afford 2-[2-(1-tert-butoxycarbonyl-4-piperidyl)ethoxy]acetic acid (4.2 g, crude) as a light yellow oil. ¹H-NMR (400 MHz, CDCl₃) δ 4.04 (s, 3H), 4.02 (s, 1H), 3.54 (t, J=6.4 Hz, 2H), 2.63 (t, J=6.4 Hz, 2H), 1.61 (d, J=12.4 Hz, 2H), 1.55-1.46 (m, 3H), 1.38 (s, 9H), 1.09-0.99 (m, 2H).

Step 3: Preparation of tert-butyl 4-[[2-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethyl]piperidine-1-carboxylate

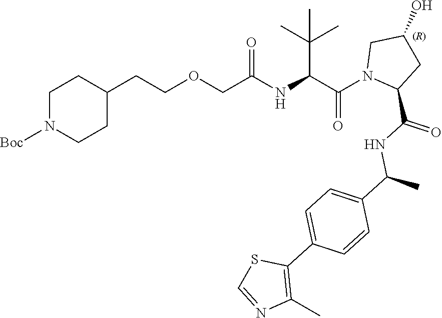

To a solution of 2-[2-(1-tert-butoxycarbonyl-4-piperidyl)ethoxy]acetic acid (400 mg, 1.39 mmol, 1.0 eq) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (619 mg, 1.39 mmol, 1.0 eq) in CH₂Cl₂ (10 mL) were added DIEA (540 mg, 4.18 mmol, 3.0 eq) and HATU (688 mg, 1.81 mmol, 1.3 eq), and the reaction mixture was stirred at 25° C. for 15 hours. The mixture was concentrated, and the resulting residue was purified by flash chromatography on silica gel (gradient: 0˜5% CH₃OH in CH₂Cl₂) to afford tert-butyl 4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethyl]piperidine-1-carboxylate (1.4 g, 1.18 mmol, 85% yield) as a light yellow oil. LC/MS (ESI) m/z: 714.2 [M+H]⁺.

Step 4: Preparation of (2S,4R)-1-[(2S)-3,3-dimethyl-2-[[2-[2-(4-piperidyl)ethoxy]acetyl]amino]butanoyl]-4-hydroxy-N-[(1S)-1-[4-(4-methylthiazol-5-yl)phenyl]ethyl]pyrrolidine-2-carboxamide

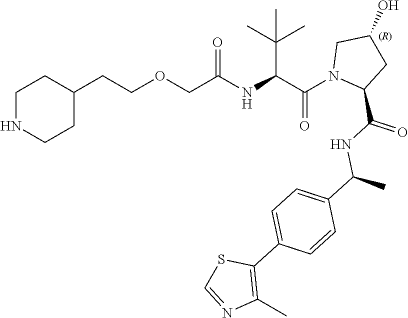

To a solution of tert-butyl 4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethyl]piperidine-1-carboxylate (1.4 g, 1.96 mmol, 1.0 eq) in CH₂Cl₂ (20 mL) was added HCl/dioxane (4 M, 15 mL), and the reaction mixture was stirred at 25° C. for 2 hours. The mixture was diluted with EtOAc (30 mL) and the pH adjusted to pH ˜8 by addition of aqueous Na₂CO₃. The organic layer was separated, washed with brine (30 mL), dried over Na₂SO₄, filtered, and concentrated to afford (2S,4R)-1-[(2S)-3,3-dimethyl-2-[[2-[2-(4-piperidyl)ethoxy]acetyl]amino]butanoyl]-4-hydroxy-N-[(1S)-1-[4-(4-methylthiazol-5-yl)phenyl]ethyl]pyrrolidine-2-carboxamide (550 mg, 851.2 umol, 43% yield) as an off-white solid. LC/MS (ESI) m/z: 614.3 [M+H]⁺.

Step 5: Preparation of tert-butyl 3-[7-(8-ethyl-3-hydroxy-1-naphthyl)-8-fluoro-2-[2-[4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethyl]-1-piperidyl]ethoxy]pyrido[4,3-d]pyrimidin-4-yl]-3,8-diazabicyclo[3.2.1]octane-8-carboxylate

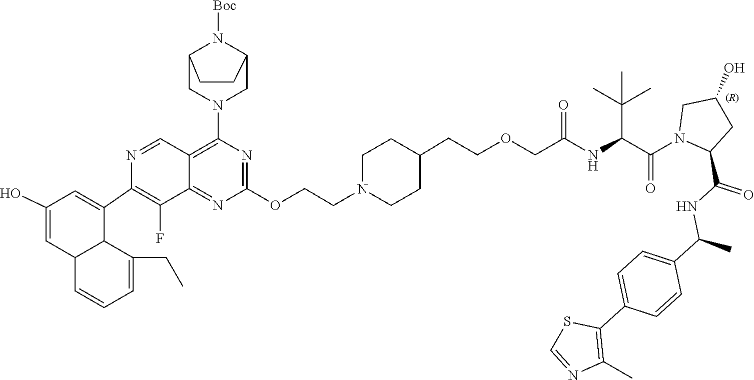

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (90 mg, 153.16 umol, 1 eq) and (2S,4R)-1-[(2S)-3,3-dimethyl-2-[[2-[2-(4-piperidyl)ethoxy]acetyl]amino]butanoyl]-4-hydroxy-N-[(1S)-1-[4-(4-methylthiazol-5-yl)phenyl]ethyl]pyrrolidine-2-carboxamide (94 mg, 153.16 umol, 1 eq) in CH₂Cl₂ (2 mL) and IPA (2 mL) were added acetic acid (46 mg, 765.78 umol, 43.80 uL, 5.0 eq) and 2-methylpyridine borane (82 mg, 765.78 umol, 5.0 eq), and the reaction mixture was stirred at 25° C. for 2 hours. The mixture was concentrated, dissolved in CH₂Cl₂ (2 mL), and then purified by flash chromatography on silica gel (gradient: 0˜7% CH₃OH in CH₂Cl₂) to afford tert-butyl 3-[7-(8-ethyl-3-hydroxy-1-naphthyl)-8-fluoro-2-[2-[4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethyl]-1-piperidyl]ethoxy]pyrido[4,3-d]pyrimidin-4-yl]-3,8-diazabicyclo[3.2.1]octane-8-carboxylate (103 mg, 64.30 umol, 42% yield) as a colorless oil. LC/MS (ESI) m/z: 1185.6 [M+H]⁺.

Step 6: Preparation of (2S,4R)-1-[(2S)-2-[[2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

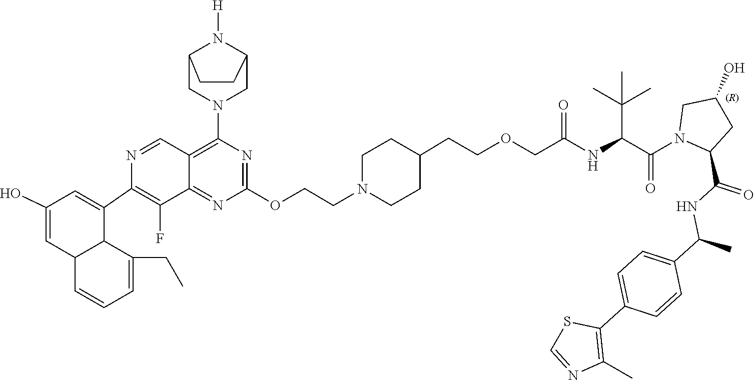

A solution of tert-butyl 3-[7-(8-ethyl-3-hydroxy-1-naphthyl)-8-fluoro-2-[2-[4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethyl]-1-piperidyl]ethoxy]pyrido[4,3-d]pyrimidin-4-yl]-3,8-diazabicyclo[3.2.1]octane-8-carboxylate (103 mg, 86.89 umol, 1.0 eq) in HCOOH (3 mL) was stirred at 25° C. for 2 hours. The mixture was concentrated, and the resulting residue was purified by prep. HPLC (5-45% CH₃CN in water (0.225% formic acid)). The pure fractions were combined and lyophilized under reduced pressure to afford product (2S,4R)-1-[(2S)-2-[2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (54.0 mg, 47.34 umol, 54% yield, formic acid salt) as a white solid. LC/MS (ESI) m/z: 1085.8. [M+H]⁺. ¹H NMR (400 MHz, CD₃OD) δ 9.07 (s, 1H), 8.87 (s, 1H), 7.62 (d, J=8.0 Hz, 1H), 7.44-7.33 (m, 5H), 7.29 (d, J=4.0 Hz, 1H), 7.17-7.15 (m, 1H), 7.00 (d, J=4.0 Hz, 1H), 5.01-4.98 (m, 1H), 4.78-4.73 (m, 3H), 4.68-4.56 (m, 7H), 4.43 (s, 1H), 3.98 (q, J=7.6 Hz, 2H), 3.85-3.72 (m, 6H), 3.62 (t, J=5.6 Hz, 2H), 3.46-3.36 (m, 2H), 3.20 (t, J=2.0 Hz, 1H), 2.62 (s, 1H), 2.46 (s, 3H), 2.39-2.19 (m, 3H), 2.04-1.79 (m, 7H), 1.63 (t, J=6.0 Hz, 2H), 1.56 (d, J=8.4 Hz, 1H), 1.50-1.42 (m, 4H), 1.03 (s, 9H), 0.91-0.86 (m, 3H).

Exemplary Synthesis of (2S,4R)-1-[(2S)-2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 12)

Step 1: Preparation of tert-butyl 4-(2-tert-butoxy-2-oxo-ethoxy) piperidine-1-carboxylate

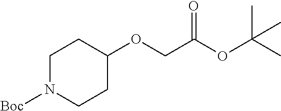 [SMILES: CC(C)(C)OC(=O)COC1CCN(C(=O)OC(C)(C)C)CC1]

To a solution of tert-butyl 4-hydroxypiperidine-1-carboxylate (5 g, 24.84 mmol, 1 eq) in THF (100 mL) at 0° C. was added NaH (1.49 g, 37.26 mmol, 60% purity, 1.5 eq), and the reaction mixture was stirred at 0° C. for 0.5 hour. tert-Butyl 2-bromoacetate (7.27 g, 37.26 mmol, 5.51 mL, 1.5 eq) was then added at 0° C., and the resulting suspension was stirred at 25° C. for 10 hours. The reaction mixture was quenched with water (100 mL) and extracted with EtOAc (3×100 mL). The combined organic extract was washed with brine (2×100 mL), dried over Na₂SO₄, filtered, and concentrated. The resulting residue was purified by flash silica gel chromatography (gradient: 0˜20% THF in petroleum ether) to give tert-butyl 4-(2-tert-butoxy-2-oxo-ethoxy) piperidine-1-carboxylate (1.5 g, 4.76 mmol, 19% yield) as a white solid. 1H NMR (400 MHz, CDCl₃) δ 4.00 (s, 2H), 3.84-3.73 (m, 2H), 3.58-3.52 (m, 1H), 3.12-3.06 (m, 2H), 1.90-1.80 (m, 2H), 1.63-1.52 (m, 2H), 1.47 (d, J=10.4 Hz, 18H).

Step 2: Preparation of 2-[(1-tert-butoxycarbonyl-4-piperidyl)oxy]acetic acid

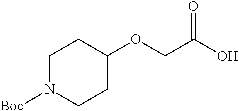 [SMILES: CC(C)(C)OC(=O)N1CCC(OCC(=O)O)CC1]

To a solution of tert-butyl 4-(2-tert-butoxy-2-oxo-ethoxy) piperidine-1-carboxylate (500 mg, 1.59 mmol, 1 eq) in H₂O (4 mL) and THF (4 mL) was added LiOH·H₂O (997.0 mg, 23.78 mmol, 15 eq), and the reaction mixture was stirred at 25° C. for 10 hours. The reaction mixture was concentrated, diluted with H₂O (10 mL), and adjusted to pH=3 with 2N HCl. The mixture was extracted with CH₂Cl₂ (3×10 mL), and the combined organic extract was washed with brine (20 mL), dried over Na₂SO₄, filtered, and concentrated to give 2-[(1-tert-butoxycarbonyl-4-piperidyl)oxy]acetic acid (350 mg, crude) as a yellow oil. ¹H NMR (400 MHz, CDCl₃) δ 4.16 (s, 2H), 3.85-3.76 (m, 2H), 3.64-3.58 (m, 1H), 3.12-3.05 (m, 2H), 1.96-1.81 (m, 2H), 1.64-1.52 (m, 2H), 1.46 (s, 9H).

Step 3: Preparation of tert-butyl 4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]piperidine-1-carboxylate

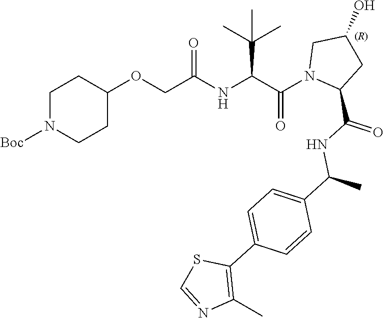

To a mixture of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (650 mg, 1.35 mmol, 1 eq, HCl) and 2-[(1-tert-butoxycarbonyl-4-piperidyl)oxy]acetic acid (350 mg, 1.35 mmol, 1 eq) in DMF (6 mL) at 25° C. were added DIEA (6.75 mmol, 1.18 mL, 5 eq) and HATU (565.0 mg, 1.48 mmol, 1.1 eq), and the reaction mixture was stirred at 25° C. for 2 hours. The reaction mixture was diluted with water (10 mL) and extracted with EtOAc (3×20 mL). The combined extract was washed with brine (3×20 mL), dried over anhydrous Na₂SO₄, filtered, and concentrated. The resulting crude product was purified by flash silica gel chromatography (gradient: 0˜60% THF in petroleum ether) to afford tert-butyl 4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]piperidine-1-carboxylate (704 mg, 821.14 umol, 61% yield) as a white solid. LC/MS (ESI) m/z: 686.3 [M+H].

Step 4: Preparation of 4-(3-azabicyclo[3.2.1]octan-3-yl)-7-[8-ethyl-3-(methoxymethoxy)-1-naphthyl]-8-fluoro-2-[2-(1-piperidyl)ethoxy]pyrido[4,3-d]pyrimidine

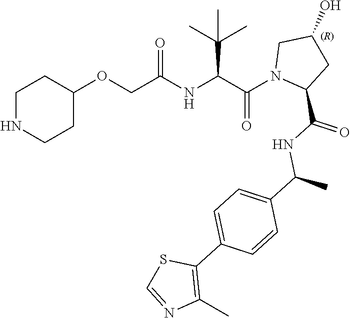

A solution of tert-butyl 4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]piperidine-1-carboxylate (500 mg, 0.73 mmol, 1 eq) in HCOOH (10 mL) was stirred at 25° C. for 1 hour. The mixture was concentrated, dissolved with H₂O (5 mL), and basified with saturated aqueous NaHCO₃ until pH=8. The resulting suspension was extracted with CH₂Cl₂ (3×20 mL), and the combined organic extract was dried over Na₂SO₄, filtered, and concentrated to give (2S,4R)-1-[(2S)-3,3-dimethyl-2-[[2-(4-piperidyloxy)acetyl]amino]butanoyl]-4-hydroxy-N-[(1S)-1-[4-(4-methylthiazol-5-yl)phenyl]ethyl]pyrrolidine-2-carboxamide (420 mg, crude) as a white solid. LC/MS (ESI) m/z: 586.2 [M+H]⁺.

Step 5: Preparation of tert-butyl 3-[7-(8-ethyl-3-hydroxy-1-naphthyl)-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]-1-piperidyl]ethoxy]pyrido[4,3-d]pyrimidin-4-yl]-3,8-diazabicyclo[3.2.1]octane-8-carboxylate

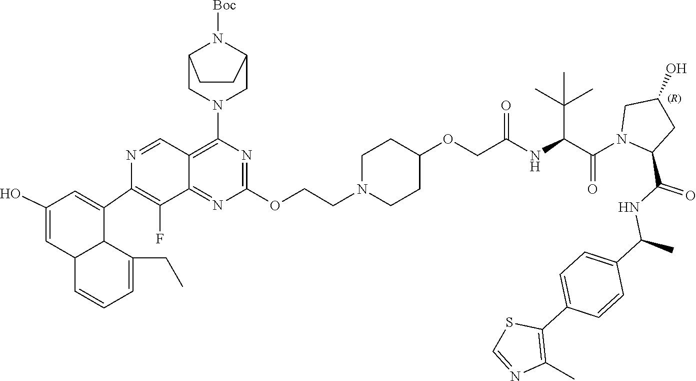

To a mixture of (2S,4R)-1-[(2S)-3,3-dimethyl-2-[[2-(4-piperidyloxy)acetyl]amino]butanoyl]-4-hydroxy-N-[(1S)-1-[4-(4-methylthiazol-5-yl)phenyl]ethyl]pyrrolidine-2-carboxamide (90.0 mg, 0.15 mmol, 1 eq) and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (90 mg, 0.15 mmol, 1 eq) in CH₂Cl₂ (2 mL) and IPA (2 mL) were added acetic acid (0.76 mmol, 44 μL, 5 eq) and 2-methylpyridine borane (82.0 mg, 0.76 mmol, 5 eq), and the reaction mixture was stirred at 25° C. for 2 hours. The reaction was concentrated, and the crude product was purified by flash silica gel chromatography (gradient: 0˜8% CH₃OH in CH₂Cl₂) to afford tert-butyl 3-[7-(8-ethyl-3-hydroxy-1-naphthyl)-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]-1-piperidyl]ethoxy]pyrido[4,3-d]pyrimidin-4-yl]-3,8-diazabicyclo[3.2.1]octane-8-carboxylate (100 mg, 86.40 umol, 56% yield) as a white solid. LC/MS (ESI) m/z: 1157.2 [M+H]⁺.

Step 6: Preparation of (2S,4R)-1-[(2S)-2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

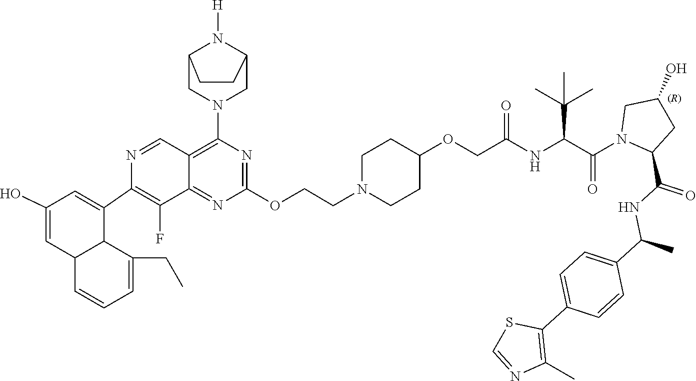

To a solution of tert-butyl 3-[7-(8-ethyl-3-hydroxy-1-naphthyl)-8-fluoro-2-[2-[4-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]-1-piperidyl]ethoxy]pyrido[4,3-d]pyrimidin-4-yl]-3,8-diazabicyclo[3.2.1]octane-8-carboxylate (100 mg, 0.086 mmol, 1 eq) in CH₂Cl₂ (1 mL) was added TFA (1 mL), and the reaction mixture was stirred at 25° C. for 0.5 hour. The mixture was concentrated, dissolved with H₂O (5 mL), and basified with saturated aqueous NaHCO₃ until pH=8. The resulting suspension was extracted with CH₂Cl₂ (3×20 mL), and the combined organic extract was dried over Na₂SO₄, filtered, and concentrated. The resulting crude product was purified by prep-HPLC (5-45% CH₃CN in water (0.225% formic acid)). Pure fraction was lyophilized to give (2S,4R)-1-[(2S)-2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]oxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (37.8 mg, 34.07 umol, 39% yield, formic acid salt) as a white solid. LC/MS (ESI) m/z: 1057.5 [M+H]⁺. ¹H NMR (400 MHz, CD₃OD) δ 9.06 (s, 1H), 8.87 (s, 1H), 8.51 (s, 1H), 7.63 (d, J=8.0 Hz, 1H), 7.45-7.40 (m, 3H), 7.39-7.32 (m, 2H), 7.29 (d, J=2.4 Hz, 1H), 7.16 (d, J=7.2 Hz, 1H), 7.01 (d, J=2.6 Hz, 1H), 5.02-4.94 (m, 1H), 4.71-4.64 (m, 6H), 4.59-4.43 (m, 3H), 4.09-3.98 (m, 2H), 3.85-3.72 (m, 6H), 3.03-3.01 (m, 4H), 2.69-2.57 (m, 2H), 2.47 (s, 3H), 2.38-2.18 (m, 3H), 2.00-1.88 (m, 7H), 1.79 (d, J=2.4 Hz, 2H), 1.50 (d, J=1.6 Hz, 3H), 1.04 (s, 9H), 0.89 (t, J=8.0 Hz, 3H).

Exemplary Synthesis of (2S,4R)-1-[(2S)-2-[[2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 11)

Step 1: Preparation of tert-butyl 4-(2-ethoxy-2-oxo-ethoxy) piperidine-1-carboxylate

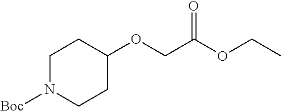 [SMILES: CCOC(=O)COC1CCN(C(=O)OC(C)(C)C)CC1]

To tert-butyl 4-hydroxypiperidine-1-carboxylate (5 g, 24.84 mmol, 1 eq) in CH₂Cl₂ (40 mL) was added Rh₂(OAc)₄ (109.0 mg, 0.25 mmol, 0.01 eq), and the resulting mixture was cooled to 0° C. A solution of ethyl 2-diazoacetate (5.6 g, 49.69 mmol, 2 eq) in CH₂Cl₂ (10 mL) was then added slowly, and the reaction mixture was stirred at 25° C. for 15 hours. The mixture was filtered through a celite pad under vacuum, and the filter cake was washed with CH₂Cl₂ (2×100 mL). The combined organic phase was concentrated, and the resulting residue was purified by flash silica gel chromatography (gradient: 0˜20% EtOAc in petroleum ether) to give tert-butyl 4-(2-ethoxy-2-oxo-ethoxy) piperidine-1-carboxylate (2 g, 6.96 mmol, 28% yield) as a colorless oil. ¹H NMR (400 MHz, CDCl₃) δ 4.23 (d, J=2.4 Hz, 2H), 4.12 (s, 2H), 3.85-3.74 (m, 2H), 3.62-3.49 (m, 1H), 3.11-3.05 (m, 2H), 1.91-1.81 (m, 2H), 1.61-1.52 (m, 2H), 1.46 (s, 9H), 1.29-1.25 (m, 3H).

Step 2: Preparation of tert-butyl 4-(2-hydroxyethoxy) piperidine-1-carboxylate

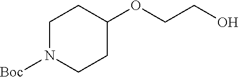 [SMILES: CC(C)(C)OC(=O)N1CCC(OCCO)CC1]

To a solution of tert-butyl 4-(2-ethoxy-2-oxo-ethoxy) piperidine-1-carboxylate (500 mg, 1.74 mmol, 1 eq) in THF (5 mL) at 0° C. was added LiAlH₄ (1 M, 4.4 mL, 2.5 eq), and the reaction mixture was stirred at 25° C. for 1 hour. The reaction mixture was quenched sequentially with H₂O (170 uL), 15% aq. NaOH (170 uL), and H₂O (510 uL). The reaction mixture was diluted with EtOAc (100 mL), and the suspension was stirred at 25° C. for 20 minutes. The mixture was dried over Na₂SO₄, filtered, and concentrated under reduced pressure to give tert-butyl 4-(2-hydroxyethoxy) piperidine-1-carboxylate (600 mg, crude) as a colorless oil. ¹H NMR (400 MHz, CDCl₃) δ 3.74 (d, J=3.6 Hz, 2H), 3.59-3.56 (m, 2H), 3.50-3.47 (m, 1H), 3.11-3.04 (m, 2H), 2.86-2.77 (m, 2H), 2.10-2.06 (m, 2H), 1.59-1.49 (m, 2H), 1.46 (s, 9H).

Step 3: Preparation of tert-butyl 4-[[2-(2-ethoxy-2-oxo-ethoxy)ethoxy]piperidine-1-carboxylate

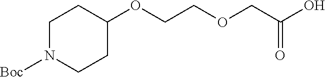 [SMILES: CC(C)(C)OC(=O)N1CCC(OCCOCC(=O)O)CC1]

To tert-butyl 4-(2-hydroxyethoxy) piperidine-1-carboxylate (600 mg, 2.45 mmol, 1 eq) in CH₂Cl₂ (8 mL) was added Rh₂(OAc)₄ (10.0 mg, 0.024 mmol, 0.01 eq), and the reaction mixture was cooled to 0° C. A solution of ethyl 2-diazoacetate (558.0 mg, 4.89 mmol, 2 eq) in CH₂Cl₂ (4 mL) was then added slowly, and the reaction mixture was stirred at 25° C. for 15 hours. The mixture was filtered through celite pad under vacuum, and the filter cake was washed with CH₂Cl₂ (2×100 mL). The combined organic extract was concentrated, and the resulting residue was purified by flash silica gel chromatography (gradient: 0˜24% EtOAc in petroleum ether) to afford tert-butyl 4-[[2-(2-ethoxy-2-oxo-ethoxy)ethoxy]piperidine-1-carboxylate (180 mg, 0.54 mmol, 22% yield) as a yellow oil. ¹H NMR (400 MHz, CDCl₃) δ 4.24-4.20 (m, 2H), 4.17-4.15 (m, 2H), 3.80-3.70 (m, 4H), 3.70-3.64 (m, 2H), 3.55-3.43 (m, 1H), 3.09-3.03 (m, 2H), 1.88-1.80 (m, 2H), 1.56-1.48 (m, 2H), 1.46 (s, 9H), 1.31-1.25 (m, 3H).

Step 4: Preparation of 2-[2-[(1-tert-butoxycarbonyl-4-piperidyl)oxy]ethoxy]acetic acid

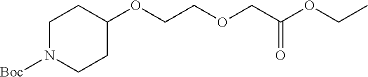 [SMILES: CCOC(=O)COCCOC1CCN(C(=O)OC(C)(C)C)CC1]

To a solution of tert-butyl 4-[[2-(2-ethoxy-2-oxo-ethoxy)ethoxy]piperidine-1-carboxylate (180 mg, 0.54 mmol, 1 eq) in H₂O (4 mL) and THF (4 mL) was added LiOH·H₂O (68.0 mg, 1.63 mmol, 3 eq), and the reaction mixture was stirred at 25° C. for 1 hour. The reaction mixture was concentrated, and the resulting residue was dissolved with H₂O (10 mL). The pH was adjusted to pH=3 by addition of 2N aqueous HCl. The aqueous mixture was extracted with CH₂Cl₂ (3×10 mL) and the combined organic extract was dried over Na₂SO₄, filtered, and concentrated to give 2-[2-[(1-tert-butoxycarbonyl-4-piperidyl)oxy]ethoxy]acetic acid (150 mg, crude) as a colorless oil. ¹H NMR (400 MHz, CDCl₃) δ 4.17 (s, 2H), 3.84-3.78 (m, 2H), 3.78-3.74 (m, 2H), 3.71-3.65 (m, 2H), 3.61-3.57 (m, 1H), 3.13-3.06 (m, 2H), 1.89-1.86 (m, 2H), 1.60-1.54 (m, 2H), 1.46 (s, 9H).

Step 5: Preparation of tert-butyl 4-[[2-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]piperidine-1-carboxylate

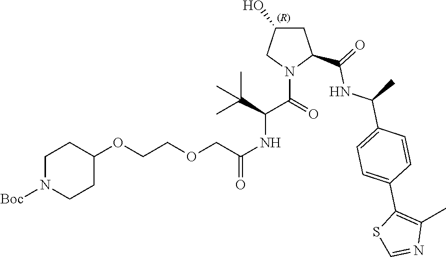

To a mixture of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (238 mg, 0.49 mmol, 1 eq, HCl) and 2-[2-[(1-tert-butoxycarbonyl-4-piperidyl)oxy]ethoxy]acetic acid (150 mg, 0.49 mmol, 1 eq) in DMF (4 mL) at 25° C. were added DIEA (319 mg, 2.47 mmol, 430.0 uL, 5 eq) and HATU (207.0 mg, 0.543 mmol, 1.1 eq), and the resulting suspension was stirred at 25° C. for 2 hours. The reaction mixture was diluted with water (10 mL) and extracted with EtOAc (3×20 mL). The combined organic extract was washed with brine (3×20 mL), dried over anhydrous Na₂SO₄, filtered, and concentrated. The crude product was purified by flash silica gel chromatography (gradient: of 0˜60% THF in petroleum ether) to give tert-butyl 4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]piperidine-1-carboxylate (340 mg) as a yellow solid. LC/MS (ESI) m/z: 730.2 [M+H]⁺.

Step 6: Preparation of (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide

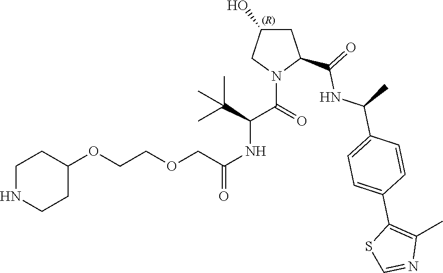

A solution of tert-butyl 4-[[2-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]piperidine-1-carboxylate (300 mg, 0.41 mmol, 1 eq) in HCOOH (5 mL) was stirred at 25° C. for 1 hour. The reaction mixture was concentrated, diluted with H₂O (2 mL), and basified with saturated aqueous NaHCO₃ until pH=8. The suspension was extracted with CH₂Cl₂ (3×20 mL), the combined organic extract was dried over Na₂SO₄, filtered, and concentrated under reduced pressure to afford (2S,4R)-1-[(2S)-3,3-dimethyl-2-[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide (200 mg, 222.29 umol, 54% yield) as a white solid. LC/MS (ESI) m/z: 630.2 [M+H].

Step 7: Preparation of tert-butyl 3-[7-(8-ethyl-3-hydroxy-1-naphthyl)-8-fluoro-2-[2-[4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]ethoxy]pyrido[4,3-d]pyrimidin-4-yl]-3,8-diazabicyclo[3.2.1]octane-8-carboxylate

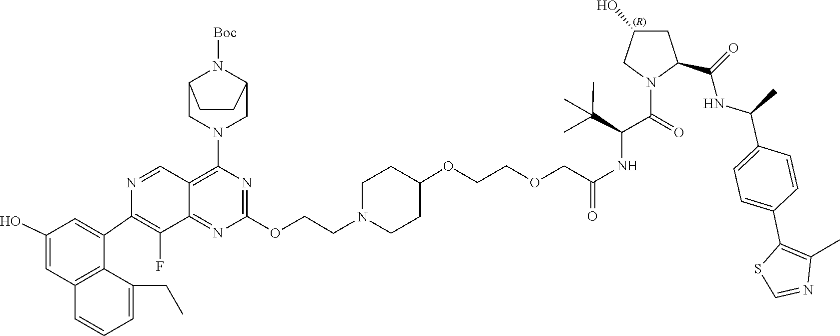

To a solution of (2S,4R)-1-[(2S)-3,3-dimethyl-2-[[2-[2-(4-piperidyloxy)ethoxy]acetyl]amino]butanoyl]-4-hydroxy-N-[(1S)-1-[4-(4-methylthiazol-5-yl)phenyl]ethyl]pyrrolidine-2-carboxamide (96 mg, 0.15 mmol, 1 eq) and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (90 mg, 0.15 mmol, 1 eq) in CH₂Cl₂ (2 mL) and IPA (2 mL) were added acetic acid (46 mg, 0.76 mmol, 44 μL, 5 eq) and 2-methylpyridine borane (82.0 mg, 0.76 mmol, 5 eq), and the reaction mixture was stirred at 25° C. for 2 hours. The reaction mixture was adjusted with TFA to pH ˜8, and then the mixture was concentrated. The crude product was purified by flash silica gel chromatography (gradient: 0˜8% CH₃OH in CH₂Cl₂) to afford tert-butyl 3-[7-(8-ethyl-3-hydroxy-1-naphthyl)-8-fluoro-2-[2-[4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]ethoxy]pyrido[4,3-d]pyrimidin-4-yl]-3,8-diazabicyclo[3.2.1]octane-8-carboxylate (150 mg, 108.62 umol, 71% yield) as a white solid confirmed. LC/MS (ESI) m/z: 1201.2 [M+H]⁺.

Step 8: Preparation of (2S,4R)-1-[(2S)-2-[[2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide

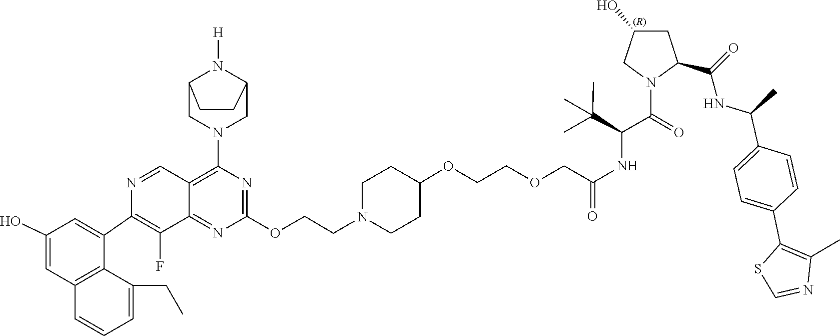

To a solution of tert-butyl 3-[7-(8-ethyl-3-hydroxy-1-naphthyl)-8-fluoro-2-[2-[4-[[2-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]ethoxy]-1-piperidyl]ethoxy]pyrido[4,3-d]pyrimidin-4-yl]-3,8-diazabicyclo[3.2.1]octane-8-carboxylate (150 mg, 0.12 mmol, 1 eq) in CH₂Cl₂ (1 mL) was added TFA (1 mL), and the resulting suspension was stirred at 25° C. for 0.5 hour. The mixture was concentrated, diluted with H₂O (5 mL), and basified with saturated aqueous NaHCO₃ until pH=8. The mixture was extracted with CH₂Cl₂ (3×10 mL) and the combined organic extract was dried over Na₂SO₄, filtered, and concentrated. The resulting crude product was purified by prep-HPLC (5-40% CH₃CN in water (0.225% formic acid)). Pure fraction was lyophilized to give (2S,4R)-1-[(2S)-2-[[2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]oxy]ethoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (56.5 mg, 47.42 umol, 38% yield, formic acid salt) as a white solid. LC/MS (ESI) m/z: 1101.3 [M+H]⁺. ¹H NMR (400 MHz, CD₃OD) δ 9.07 (s, 1H), 8.91-8.79 (m, 1H), 8.49 (s, 1H), 7.63 (d, J=8.0 Hz, 1H), 7.45-7.32 (m, 5H), 7.29 (d, J=2.4 Hz, 1H), 7.15 (d, J=7.2 Hz, 1H), 7.01 (s, 1H), 5.01-4.96 (m, 1H), 4.76-4.62 (m, 5H), 4.59-4.50 (m, 1H), 4.43 (s, 1H), 4.05 (d, J=2.0 Hz, 2H), 3.91-3.36 (m, 12H), 3.22 (s, 4H), 2.90-2.88 (m, 1H), 2.45 (s, 3H), 2.40-2.15 (m, 3H), 2.08-1.77 (m, 9H), 1.60-1.43 (m, 3H), 1.04 (s, 9H), 0.88 (d, J=t, 8.0 Hz, 3H).

Exemplary Synthesis of (2S,4S)-1-[(2S)-2-[[2-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 10)

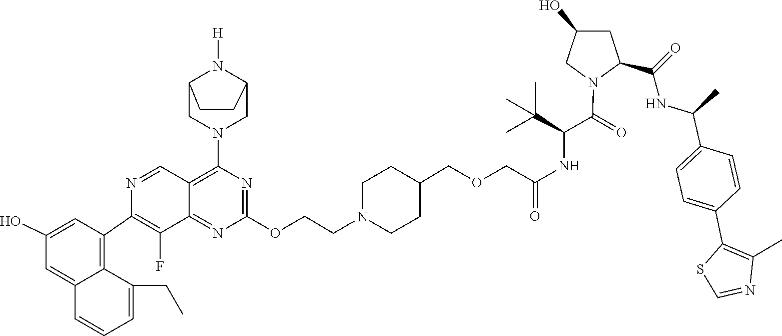 [SMILES: CCc1cccc2cc(O)cc(-c3ncc4c(N5CC6CCC(C5)N6)nc(OCCN5CCC(COCC(=O)N[C@H](C(=O)N6C[C@@H](O)C[C@H]6C(=O)N[C@@H](C)c6ccc(-c7scnc7C)cc6)C(C)(C)C)CC5)nc4c3F)c12]

This compound was prepared in an analogous manner to Compound 21 starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4S)-1-[(2S)-3,3-dimethyl-2-[[2-(4-piperidylmethoxy)acetyl]amino]butanoyl]-4-hydroxy-N-[(1S)-1-[4-(4-methylthiazol-5-yl)phenyl]ethyl]pyrrolidine-2-carboxamide.

(white solid, formic acid salt). LC/MS (ESI) m/z: 1071.8 [M+H]⁺. ¹H NMR (400 MHz, CD₃OD) δ 9.07 (s, 1H), 8.89-8.83 (m, 1H), 8.51 (s, 1H), 7.63 (d, J=8.0 Hz, 1H), 7.44-7.33 (m, 5H), 7.30-7.27 (m, 1H), 7.16 (d, J=7.2 Hz, 1H), 7.01 (t, J=2.4 Hz, 1H), 5.01-4.97 (m, 1H), 4.77-4.70 (m, 3H), 4.69-4.58 (m, 5H), 4.51-4.44 (m, 1H), 4.41-4.32 (m, 1H), 4.00-3.93 (m, 2H), 3.84-3.66 (m, 5H), 3.43 (d, J=6.0 Hz, 2H), 3.41-3.33 (m, 2H), 3.16 (s, 1H), 2.60-2.54 (m, 1H), 2.47 (s, 3H), 2.44-2.22 (m, 3H), 1.98-1.80 (m, 8H), 1.56-1.42 (m, 5H), 1.04-0.97 (m, 9H), 0.89 (t, J=7.6 Hz, 3H).

Exemplary Synthesis of (2S,4R)-1-[(2S)-2-[[1-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]cyclopropanecarbonyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 9)

Step 1: Preparation of tert-butyl 4-(trifluoromethylsulfonyloxymethyl) piperidine-1-carboxylate

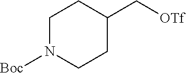 [SMILES: CC(C)(C)OC(=O)N1CCC(COS(=O)(=O)C(F)(F)F)CC1]

To a solution of tert-butyl 4-(hydroxymethyl) piperidine-1-carboxylate (400 mg, 1.86 mmol, 1 eq) and 2,6-dimethylpyridine (219 mg, 2.04 mmol, 1.1 eq) in CH₂Cl₂ (20 mL) at −78° C. was added Tf₂O (577 mg, 2.04 mmol, 1.1 eq) dropwise under nitrogen, and the reaction mixture was stirred at −78° C. for 1 hour. The reaction mixture was diluted with CH₂Cl₂ (25 mL) and washed sequentially with saturated NH₄Cl (25 mL) and water (25 mL). The organic layer was dried over anhydrous Na₂SO₄, filtered, and concentrated to afford tert-butyl 4-(trifluoromethylsulfonyloxymethyl) piperidine-1-carboxylate (641 mg, 1.85 mmol, 99% yield) as a red oil. ¹H NMR (400 MHz, CDCl₃) δ 4.30 (d, J=6.4 Hz, 2H), 4.11 (d, J=12.8 Hz, 2H), 2.65 (t, J=12.0 Hz, 2H), 2.10-1.90 (m, 1H), 1.78 (d, J=13.2 Hz, 2H), 1.47 (s, 9H), 1.32-1.18 (m, 2H).

Step 2: Preparation of tert-butyl 4-[(1-ethoxycarbonylcyclopropoxy)methyl]piperidine-1-carboxylate

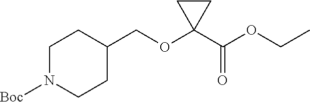 [SMILES: CCOC(=O)C1(OCC2CCN(C(=O)OC(C)(C)C)CC2)CC1]

To a solution of ethyl 1-hydroxycyclopropanecarboxylate (200 mg, 1.54 mmol, 1 eq) in THF (5 mL) at −78° C. was added LiHMDS (1 M, 1.84 mL, 1.2 eq), and the reaction mixture was stirred at −78° C. under N₂ for 1 hour. A solution of tert-butyl 4-(trifluoromethylsulfonyloxymethyl) piperidine-1-carboxylate (640.57 mg, 1.84 mmol, 1.2 eq) in THF (5 mL) was then added, and the reaction mixture was warmed to 25° C. and stirred at 25° C. for 16 hours. The reaction mixture was quenched by addition of saturated aqueous NH₄Cl (20 mL) and extracted with EtOAc (3×20 mL). The combined organic extract was washed with brine (2×20 mL), dried over anhydrous Na₂SO₄, filtered, and concentrated to give tert-butyl 4-[(1-ethoxycarbonylcyclopropoxy)methyl]piperidine-1-carboxylate (785 mg, crude) as a yellow oil.

Step 3: Preparation of 1-[(1-tert-butoxycarbonyl-4-piperidyl)methoxy]cyclopropanecarboxylic acid

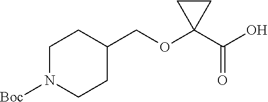 [SMILES: CC(C)(C)OC(=O)N1CCC(COC2(C(=O)O)CC2)CC1]

To a solution of tert-butyl 4-[(1-ethoxycarbonylcyclopropoxy)methyl]piperidine-1-carboxylate (785 mg, 2.40 mmol, 1 eq) in THF (2 mL), EtOH (2 mL), and H₂O (2 mL) was added LiOH·H₂O (201 mg, 4.80 mmol, 2 eq), and the reaction mixture was stirred at 25° C. under N₂ for 4 hours. The reaction mixture was concentrated under reduced pressure, and the resulting residue was dissolved with water (10 mL) and CH₂Cl₂ (5 mL). The organic layer was separated, and the aqueous layer was adjusted to pH=6 with 2N aq. HCl, then dried by lyophilization. The resulting crude product was triturated with CH₂Cl₂/CH₃OH (30 mL, 10/1) at 25° C. for 10 min. The suspension was filtered, and the filtrate was concentrated under reduced pressure give to 1-[(1-tert-butoxycarbonyl-4-piperidyl)methoxy]cyclopropanecarboxylic acid (250 mg, 0.835 mmol, 35% yield) as a colorless oil.

Step 4: Preparation of tert-butyl 4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]piperidine-1-carboxylate

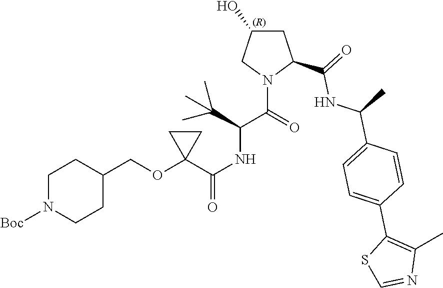

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (400 mg, 0.832 mmol, 1 eq, HCl) and 1-[(1-tert-butoxycarbonyl-4-piperidyl)methoxy]cyclopropanecarboxylic acid (249 mg, 0.832 mmol, 1 eq) in DMF (5 mL) were added DIEA (537 mg, 4.16 mmol, 5 eq) and HATU (411 mg, 1.08 mmol, 1.3 eq), and the reaction mixture was stirred at 25° C. for 2 hours. The reaction mixture was quenched by addition of water (50 mL) and the resulting mixture was stirred at 25° C. for 10 minutes. The resulting suspension was filtered, and the cake was dissolved in CH₂Cl₂ (30 mL). The organic layer was dried over anhydrous Na₂SO₄, filtered, and concentrated, and the resulting crude product was purified by flash silica gel chromatography (gradient: 0˜60% THF in petroleum ether) to give tert-butyl 4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]piperidine-1-carboxylate (411 mg, 0.193 mmol, 23% yield) as a colorless gum. LC/MS (ESI) m/z: 726.3 [M+H]⁺.

Step 5: Preparation of (2S,4R)-1-[(2S)-3,3-dimethyl-2-[[1-(4-piperidylmethoxy)cyclopropanecarbonyl]amino]butanoyl]-4-hydroxy-N-[(1S)-1-[4-(4-methylthiazol-5-yl)phenyl]ethyl]pyrrolidine-2-carboxamide

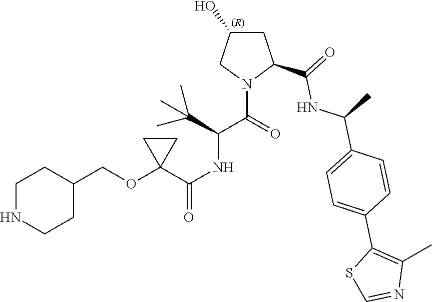

A solution of tert-butyl 4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]piperidine-1-carboxylate (150 mg, 0.207 mmol, 1 eq) in CH₂Cl₂ (1.5 mL) and TFA (0.5 mL) was stirred at 25° C. for 2 hours. The reaction mixture was concentrated, and the resulting residue was dissolved in water (5 mL). The resulting aqueous mixture was adjusted to pH=9 with saturated Na₂CO₃ solution, then extracted with CH₃OH/CH₂Cl₂ (3×30 mL). The combined organic extract was washed with brine (30 mL), dried over anhydrous Na₂SO₄, filtered, and concentrated under reduced pressure to give (2S,4R)-1-[(2S)-3,3-dimethyl-2-[[1-(4-piperidylmethoxy)cyclopropanecarbonyl]amino]butanoyl]-4-hydroxy-N-[(1S)-1-[4-(4-methylthiazol-5-yl)phenyl]ethyl]pyrrolidine-2-carboxamide (125 mg) as a colorless gum. LC/MS (ESI) m/z: 626.2 [M+H]⁺.

Step 6: Preparation of tert-butyl 3-[7-(8-ethyl-3-hydroxy-1-naphthyl)-8-fluoro-2-[2-[4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]-1-piperidyl]ethoxy]pyrido[4,3-d]pyrimidin-4-yl]-3,8-diazabicyclo[3.2.1]octane-8-carboxylate

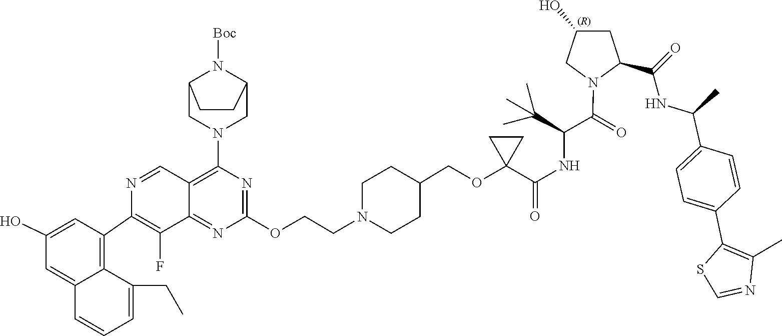

To a solution of (2S,4R)-1-[(2S)-3,3-dimethyl-2-[[1-(4-piperidylmethoxy)cyclopropanecarbonyl]amino]butanoyl]-4-hydroxy-N-[(1S)-1-[4-(4-methylthiazol-5-yl)phenyl]ethyl]pyrrolidine-2-carboxamide (125 mg, 0.200 mmol, 1 eq) and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (100 mg, 0.170 mmol, 0.85 eq) in IPA (1 mL)/CH₂Cl₂ (2 mL) were added acetic acid (48 mg, 0.80 mmol, 4 eq) and 2-methylpyridine borane (107 mg, 0.999 mmol, 5 eq), and the reaction mixture was stirred at 25° C. under N₂ for 0.5 hour. The reaction mixture was concentrated, and the crude product was purified by prep-HPLC (30-90% CH₃CN in water (NH₄HCO₃)). The pure fractions were combined and dried by lyophilization to give tert-butyl 3-[7-(8-ethyl-3-hydroxy-1-naphthyl)-8-fluoro-2-[2-[4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]-1-piperidyl]ethoxy]pyrido[4,3-d]pyrimidin-4-yl]-3,8-diazabicyclo[3.2.1]octane-8-carboxylate (71 mg, 0.059 mmol, 30% yield) as a white solid. LC/MS (ESI) m/z: 1198.5 [M+H]⁺.

Step 7: Preparation of (2S,4R)-1-[(2S)-2-[[1-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]cyclopropanecarbonyl]amino]-3,3-dimethyl-butanoyl]-4-hydroxy-N-[(1S)-1-[4-(4-methylthiazol-5-yl)phenyl]ethyl]pyrrolidine-2-carboxamide

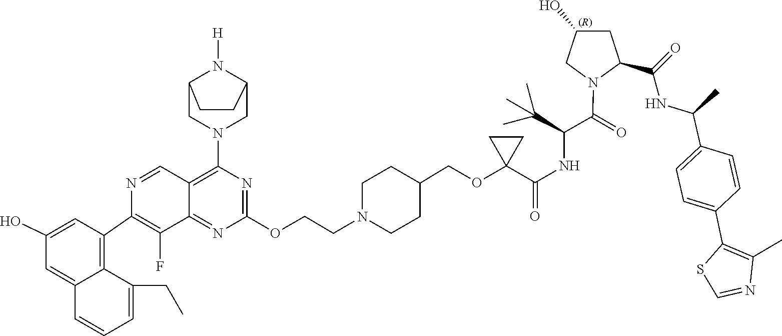

To a solution of tert-butyl 3-[7-(8-ethyl-3-hydroxy-1-naphthyl)-8-fluoro-2-[2-[4-[[1-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclopropoxy]methyl]-1-piperidyl]ethoxy]pyrido[4,3-d]pyrimidin-4-yl]-3,8-diazabicyclo[3.2.1]octane-8-carboxylate (71 mg, 0.059 mmol, 1 eq) in CH₂Cl₂ (1.5 mL) was added TFA (0.5 mL), and the reaction mixture was stirred at 25° C. for 1 hour. The reaction mixture was concentrated under reduced pressure to give a residue. Saturated aqueous NaHCO₃ (1 mL) and EtOAc/THF (30 mL, 2/1) were then added, and the resulting mixture was dried over anhydrous Na₂SO₄, filtered, and concentrated. The crude product was purified by prep-HPLC (5-45% CH₃CN in water (0.225% formic acid)). The pure fractions were combined and dried by lyophilization to give (2S,4R)-1-[(2S)-2-[[1-[[1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]cyclopropanecarbonyl]amino]-3,3-dimethyl-butanoyl]-4-hydroxy-N-[(1S)-1-[4-(4-methylthiazol-5-yl)phenyl]ethyl]pyrrolidine-2-carboxamide (40.4 mg, 0.034 mmol, 58% yield, formic acid) as a white solid. LC/MS (ESI) m/z: 1097.5 [M+H]⁺. ¹H NMR (400 MHz, CD₃OD) δ 9.10 (s, 1H), 8.92-8.86 (m, 1H), 8.51 (s, 1H), 7.65 (d, J=8.0 Hz, 1H), 7.51-7.37 (m, 5H), 7.35-7.29 (m, 1H), 7.18 (d, J=6.8 Hz, 1H), 7.03 (d, J=2.4 Hz, 1H), 5.01-4.98 (m, 1H), 4.81-4.75 (m, 2H), 4.73-4.71 (m, 1H), 4.70-4.68 (m, 1H), 4.63-4.59 (m, 1H), 3.99-3.71 (m, 6H), 3.62-3.36 (m, 6H), 3.32-3.23 (m, 2H), 2.88-2.67 (m, 2H), 2.48 (s, 3H), 2.41-2.21 (m, 3H), 2.04-1.80 (m, 8H), 1.66-1.46 (m, 5H), 1.25-1.02 (m, 13H), 0.91 (t, J=7.2 Hz, 3H).

Exemplary Synthesis of (2S,4R)-1-[(2R)-2-[3-[7-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 16)

Step 1: Preparation of methyl 3-methyl-2-[3-(1,1,2,2,3,3,4,4,4-nonafluorobutylsulfonyloxy)isoxazol-5-yl]butanoate

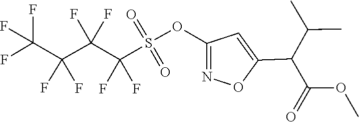 [SMILES: COC(=O)C(c1cc(OS(=O)(=O)C(F)(F)C(F)(F)C(F)(F)C(F)(F)F)no1)C(C)C]

To a mixture of methyl 2-(3-hydroxyisoxazol-5-yl)-3-methyl-butanoate (20.0 g, 100 mmol, 1 eq) and K₂CO₃ (6.94 g, 50.2 mmol, 0.5 eq) in CH₃CN (100 mL) at 0° C. was added 1,1,2,2,3,3,4,4,4-nonafluorobutane-1-sulfonyl fluoride (60.7 g, 201 mmol, 2 eq) dropwise under N₂, and the reaction mixture was stirred at 20° C. for 12 hours. The mixture was diluted with EtOAc (100 mL) and washed with saturated NH₄Cl solution (100 mL). The aqueous phase was extracted with EtOAc (3×120 mL), and the organic extract was washed with brine (100 mL), dried over anhydrous Na₂SO₄, and concentrated. The resulting residue was purified by flash chromatography on SiO₂ (gradient: 0-1% THF in petroleum ether) to afford methyl 3-methyl-2-[3-(1,1,2,2,3,3,4,4,4-nonafluorobutylsulfonyloxy)isoxazol-5-yl]butanoate (37.4 g, 71.5 mmol, 71% yield) as a colorless oil. LC/MS (ESI) m/z: 481.8 [M+H]⁺.

Step 2: Preparation of tert-butyl 2-[5-(1-methoxycarbonyl-2-methyl-propyl)isoxazol-3-yl]-2,7-diazaspiro[3.5]nonane-7-carboxylate

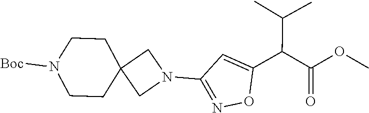 [SMILES: COC(=O)C(c1cc(N2CC3(CCN(C(=O)OC(C)(C)C)CC3)C2)no1)C(C)C]

To a solution of tert-butyl 2,7-diazaspiro[3.5]nonane-7-carboxylate (3.00 g, 13.3 mmol, 1 eq) in DMA (60 mL) were added diisopropylethylamine (5.14 g, 39.8 mmol, 3 eq) and methyl 3-methyl-2-[3-(1,1,2,2,3,3,4,4,4-nonafluorobutylsulfonyloxy)isoxazol-5-yl]butanoate (7.66 g, 15.9 mmol, 1.2 eq), and the reaction mixture was stirred at 140° C. for 12 hours under N₂. The resulting residue was purified by prep-HPLC (30-70% CH₃CN in water (0.05% HCl)) to afford tert-butyl 2-[5-(1-methoxycarbonyl-2-methyl-propyl)isoxazol-3-yl]-2,7-diazaspiro[3.5]nonane-7-carboxylate (1.13 g, 2.63 mmol, 20% yield) as a white solid. LC/MS (ESI) m/z: 408.0 [M+H]⁺.

Step 3: Preparation of 2-[3-(7-tert-butoxycarbonyl-2,7-diazaspiro[3.5]nonan-2-yl) isoxazol-5-yl]-3-methyl-butanoic acid

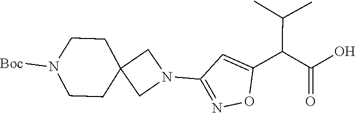 [SMILES: CC(C)C(C(=O)O)c1cc(N2CC3(CCN(C(=O)OC(C)(C)C)CC3)C2)no1]

To tert-butyl 2-[5-(1-methoxycarbonyl-2-methyl-propyl)isoxazol-3-yl]-2,7-diazaspiro[3.5]nonane-7-carboxylate (1.13 g, 2.77 mmol, 1 eq) in THF (10 mL), CH₃OH (10 mL) and H₂O (10 mL) was added LiOH·H₂O (582 mg, 13.9 mmol, 5 eq), and the reaction mixture was stirred at 20° C. for 1 hour. The reaction mixture was adjusted to pH=3 with aq. HCl (2N), and then lyophilized to give 2-[3-(7-tert-butoxycarbonyl-2,7-diazaspiro[3.5]nonan-2-yl)isoxazol-5-yl]-3-methyl-butanoic acid (1.09 g, crude) as a white solid. LC/MS (ESI) m/z: 394.0 [M+H]⁺.

Step 4: Preparation of tert-butyl 2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate

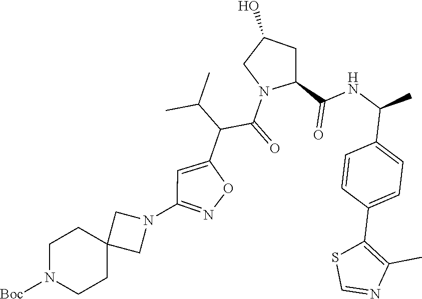 [SMILES: Cc1ncsc1-c1ccc([C@H](C)NC(=O)[C@@H]2C[C@@H](O)CN2C(=O)C(c2cc(N3CC4(CCN(C(=O)OC(C)(C)C)CC4)C3)no2)C(C)C)cc1]

To a mixture of 2-[3-(7-tert-butoxycarbonyl-2,7-diazaspiro[3.5]nonan-2-yl)isoxazol-5-yl]-3-methyl-butanoic acid (1.09 g, 2.77 mmol, 1 eq) and (2S,4R)-4-hydroxy-N-[(1S)-1-[4-(4-methylthiazol-5-yl)phenyl]ethyl]pyrrolidine-2-carboxamide (1.02 g, 2.77 mmol, 1 eq, HCl salt) in DMF (10 mL) were added diisopropylethylamine (1.79 g, 13.9 mmol, 5 eq) and HATU (1.05 g, 2.77 mmol, 1 eq), and the reaction mixture was stirred at 20° C. for 2 hours. Water (20 mL) was then added, and the resulting mixture was extracted with EtOAc (3×20 mL). The combined organic extract was washed with brine (3×20 mL), dried over Na₂SO₄, and concentrated. The resulting residue was purified by flash chromatography on SiO₂ (gradient: 0-50% THF in petroleum ether) to afford tert-butyl 2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (1.96 g, 2.33 mmol, 84% purity) as a yellow solid. LC/MS (ESI) m/z: 707.5 [M+H]⁺.

Step 5: Chiral separation of tert-butyl 2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate

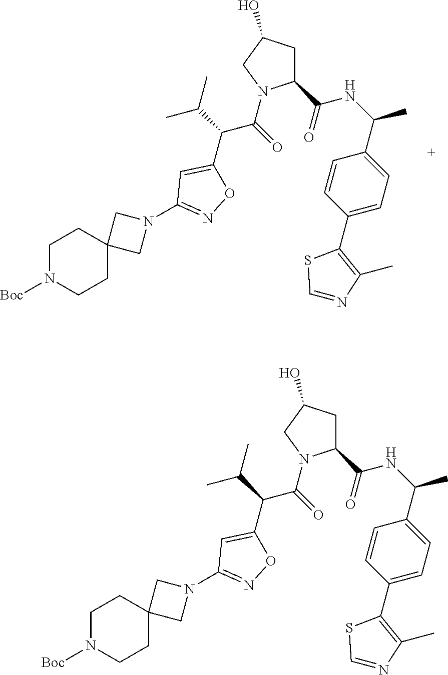 [SMILES: Cc1ncsc1-c1ccc([C@H](C)NC(=O)[C@@H]2C[C@@H](O)CN2C(=O)[C@H](c2cc(N3CC4(CCN(C(=O)OC(C)(C)C)CC4)C3)no2)C(C)C)cc1 ; Cc1ncsc1-c1ccc([C@H](C)NC(=O)[C@@H]2C[C@@H](O)CN2C(=O)[C@@H](c2cc(N3CC4(CCN(C(=O)OC(C)(C)C)CC4)C3)no2)C(C)C)cc1]

tert-butyl 2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (1.70 g, 2.40 mmol, 1 eq) was separated by SFC (40% isopropanol in water (0.1% NH₄OH)) to afford tert-butyl 2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (800 mg, 0.985 mmol, 41% yield, 87% purity) as a white solid (LC/MS (ESI) m/z: 707.3 [M+H]⁺) and tert-butyl 2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (760 mg, 1.02 mmol, 42% yield) as a white solid (LC/MS (ESI) m/z: 707.4 [M+H]⁺).

Step 6: Preparation of (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide

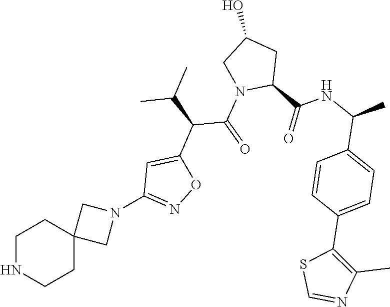 [SMILES: Cc1ncsc1-c1ccc([C@H](C)NC(=O)[C@@H]2C[C@@H](O)CN2C(=O)[C@@H](c2cc(N3CC4(CCNCC4)C3)no2)C(C)C)cc1]

To a solution of tert-butyl 2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonane-7-carboxylate (760 mg, 1.08 mmol, 1 eq) in CH₂Cl₂ (3 mL) was added TFA (1 mL), and the reaction mixture was stirred at 20° C. under N₂ for 2.5 hours. The reaction mixture was concentrated under reduced pressure, and the resulting residue was diluted with water (10 mL). The resulting aqueous mixture was adjusted to pH=9 with saturated aqueous NaHCO₃ and extracted with 20:1 CH₂Cl₂/CH₃OH (3×20 mL). The combined organic extract was dried over anhydrous Na₂SO₄, filtered, and concentrated to give (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (646 mg, 0.905 mmol, 85% purity) as a white solid. LC/MS (ESI) m/z: 607.4 [M+H]⁺.

Step 7: Preparation of tert-butyl 3-[7-(8-ethyl-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate

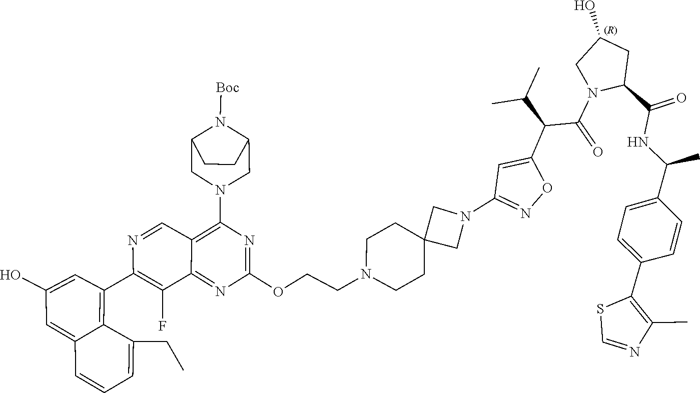

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (80 mg, 0.136 mmol, 1.0 eq) and (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (124 mg, 0.163 mmol, 79.8% purity, 1.2 eq) in CH₂Cl₂ (2 mL) and IPA (2 mL) were added acetic acid (31 uL, 0.545 mmol, 4.0 eq) and 2-methylpyridine borane (72.8 mg, 0.681 mmol, 5.0 eq), and the reaction mixture was stirred at 25° C. for 1 hour. Purification by flash chromatography on silica gel (gradient: 0˜7% CH₃OH in CH₂Cl₂) afforded tert-butyl 3-[7-(8-ethyl-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (125 mg, 0.0943 mmol, 69% yield) as a white solid. LC/MS (ESI) m/z: 589.9 [M/2+H]⁺.

Step 8: Preparation of (2S,4R)-1-[(2R)-2-[3-[7-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

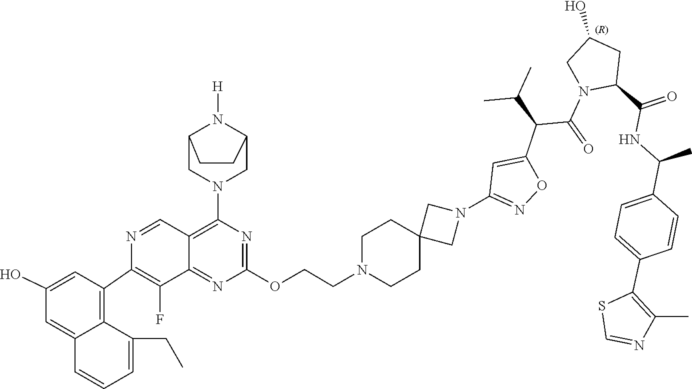

A solution of tert-butyl 3-[7-(8-ethyl-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (120 mg, 0.102 mmol, 1.0 eq) in HCOOH (3 mL) was stirred at 20° C. for 2 hours. The crude product was purified by prep-HPLC (5-40% CH₃CN in water (0.225% formic acid)). The pure fractions were combined and lyophilized to afford (2S,4R)-1-[(2R)-2-[3-[7-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (44.1 mg, 0.0406 mmol, 40% yield) as a white solid. LC/MS (ESI) m/z: 540.3 [M/2+H]⁺. ¹H NMR (400 MHz, CD₃OD) δ 9.08 (s, 1H), 8.88 (s, 1H), 7.64 (d, J=8.4 Hz, 1H), 7.46-7.35 (m, 5H), 7.30 (s, 1H), 7.19-7.14 (d, J=7.2 Hz, 1H), 7.01 (s, 1H), 5.88 (s, 1H), 5.07-4.99 (m, 1H), 4.74-4.70 (m, 2H), 4.53-4.38 (m, 2H), 4.03-3.95 (m, 2H), 3.88-3.78 (m, 3H), 3.72-3.58 (m, 6H), 3.01-3.00 (m, 2H), 2.94-2.70 (m, 4H), 2.48 (s, 3H), 2.42-2.12 (m, 5H), 2.07-1.85 (m, 10H), 1.61-1.51 (m, 3H), 1.07-1.02 (m, 3H), 0.93-0.87 (m, 6H).

Exemplary Synthesis of (2S,4R)-1-[(2S)-2-[3-[7-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 15)

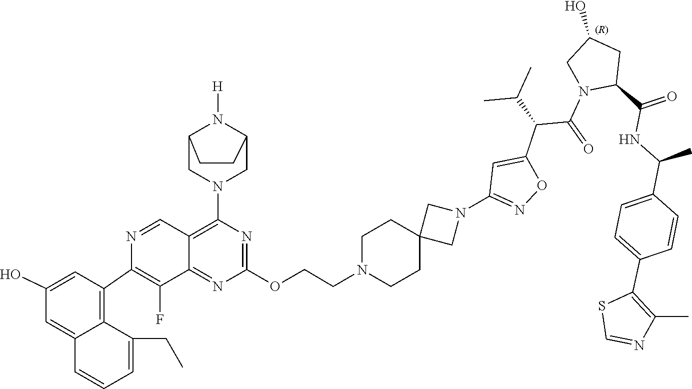

This compound was prepared in an analogous manner to Compound 16 starting from 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (80 mg, 0.136 mmol, 1.0 eq) and (2S,4R)-1-[(2S)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide.

(white solid, formic acid salt). LC/MS (ESI) m/z: 1078.5 [M+H]⁺. ¹H NMR (400 MHz, CD₃OD) δ 9.07 (s, 1H), 8.89-8.85 (m, 1H), 8.47 (d, J=13.2 Hz, 1H), 7.63 (d, J=8.0 Hz, 1H), 7.46 (d, J=3.2 Hz, 1H), 7.45-7.39 (m, 1H), 7.39-7.32 (m, 3H), 7.29 (s, 1H), 7.16 (d, J=6.8 Hz, 1H), 7.01 (d, J=2.8 Hz, 1H), 5.89 (s, 1H), 5.00-4.96 (m, 1H), 4.78-4.68 (m, 5H), 4.63-4.54 (m, 2H), 4.43 (s, 1H), 3.99 (s, 2H), 3.89-3.78 (m, 2H), 3.73-3.65 (m, 6H), 3.07 (s, 2H), 2.82 (d, J=1.2 Hz, 3H), 2.49-2.45 (m, 3H), 2.27-2.25 (m, 1H), 2.39-2.20 (m, 4H), 2.04-1.88 (m, 9H), 1.49 (d, J=7.2 Hz, 3H), 1.05 (d, J=6.4 Hz, 3H), 0.93-0.86 (m, 6H).

Exemplary Synthesis of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl-4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 5)

Step 1: Preparation of 1-benzyl 2-methyl (2S)-pyrrolidine-1,2-dicarboxylate

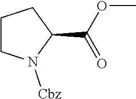 [SMILES: COC(=O)[C@@H]1CCCN1C(=O)OCc1ccccc1]

To a solution of methyl (2S)-pyrrolidine-2-carboxylate hydrochloride (40.0 g, 242 mmol, 1 eq) in CH₂Cl₂ (500 mL) were added TEA (56.2 g, 556 mmol, 2.3 eq) and CbzCl (53.6 g, 314 mmol, 1.3 eq), and the reaction mixture was stirred at 25° C. under N₂ for 16 hours. The reaction mixture was washed with water (100 mL), brine (100 mL), dried over anhydrous Na₂SO₄, filtered, and concentrated. The crude product was purified by flash silica gel chromatography (gradient: 0˜10% THF in petroleum ether) to give O1-benzyl O2-methyl (2S)-pyrrolidine-1,2-dicarboxylate (60.6 g, 230 mmol, 95% yield) as a colorless oil. LC/MS (ESI) m/z: 264.0 [M+H]⁺.

Step 2: Preparation of 1-benzyl 2-methyl 2-but-3-enylpyrrolidine-1,2-dicarboxylate

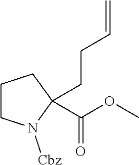 [SMILES: C=CCCC1(C(=O)OC)CCCN1C(=O)OCc1ccccc1]

To a stirred solution of O1-benzyl O2-methyl (2S)-pyrrolidine-1,2-dicarboxylate (27.5 g, 104 mmol, 1 eq) in THF (300 mL) at −78° C. was added LiHMDS (1 M, 125 mL, 1.2 eq), and the reaction mixture was stirred at −78° C. under N₂ for 0.5 hour. 4-Bromobut-1-ene (28.2 g, 209 mmol, 2 eq) was then added at −78° C., and the reaction mixture was stirred at 20° C. under N₂ for 16 hours. Two batches were conducted. The reaction mixture was quenched by addition of saturated NH₄Cl solution (300 mL) and extracted with EtOAc (2×300 mL). The combined organic extract was washed with brine (300 mL), dried over anhydrous Na₂SO₄, filtered, and concentrated. The crude product was purified by flash silica gel chromatography (gradient: 0˜8% THF in petroleum ether) to give O1-benzyl O2-methyl 2-but-3-enylpyrrolidine-1,2-dicarboxylate (44.9 g, 141 mmol, 68% yield) as a yellow oil. LC/MS (ESI) m/z: 318.0 [M+H]⁺.

Step 3: Preparation of 1-benzyl 2-methyl 2-[2-(oxiran-2-yl)ethyl]pyrrolidine-1,2-dicarboxylate

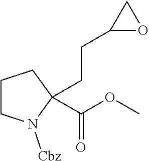 [SMILES: COC(=O)C1(CCC2CO2)CCCN1C(=O)OCc1ccccc1]

To a solution of O1-benzyl O2-methyl 2-but-3-enylpyrrolidine-1,2-dicarboxylate (44.9 g, 141 mmol, 1 eq) in CH₂Cl₂ (600 mL) was added m-CPBA (33.6 g, 156 mmol, 80% purity, 1.1 eq), and the reaction mixture was stirred at 20° C. for 16 hours. The reaction mixture was filtered, and the filtrate was washed with saturated aqueous NaHSO₃ (300 mL), NaHCO₃ (2×200 mL), and brine (200 mL), then dried over anhydrous Na₂SO₄, filtered, and concentrated. The crude product was purified by flash silica gel chromatography (gradient: 0˜15% THF in petroleum ether) to give O1-benzyl O2-methyl 2-[2-(oxiran-2-yl)ethyl]pyrrolidine-1,2-dicarboxylate (37.5 g, 112 mmol, 80% yield) as a colorless oil. LC/MS (ESI) m/z: 334.2 [M+H]⁺.

Step 4: Preparation of methyl 3-(hydroxymethyl)-1,2,3,5,6,7-hexahydropyrrolizine-8-carboxylate

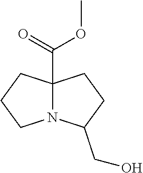

To a solution of O1-benzyl O2-methyl 2-[2-(oxiran-2-yl)ethyl]pyrrolidine-1,2-dicarboxylate (37.5 g, 112 mmol, 1 eq) in CH₃OH (400 mL) was added Pd/C (3.0 g, 10% purity), and the reaction mixture was stirred at 25° C. under H₂ (15 psi) (degassed under vacuum and purged with H₂ several times) for 16 hours. The reaction mixture was filtered, and the filtrate was concentrated under reduced pressure to give methyl 3-(hydroxymethyl)-1,2,3,5,6,7-hexahydropyrrolizine-8-carboxylate (22.4 g, 112 mmol, 100% yield) as a yellow oil.

Step 5: Preparation of methyl 3-[[tert-butyl(diphenyl)silyl]oxymethyl]-1,2,3,5,6,7-hexahydropyrrolizine-8-carboxylate

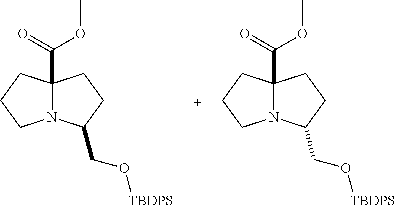

To a solution of methyl 3-(hydroxymethyl)-1,2,3,5,6,7-hexahydropyrrolizine-8-carboxylate (22.4 g, 112 mmol, 1 eq) and imidazole (9.95 g, 146 mmol, 1.3 eq) in CH₂Cl₂ (300 mL) was added TBDPSCl (37.1 g, 135 mmol, 1.2 eq), and the reaction mixture was stirred at 25° C. under N₂ for 16 hours. The reaction mixture was quenched by addition of water (100 mL) and extracted with CH₂Cl₂ (3×100 mL). The combined organic extract was washed with brine (2×100 mL), dried over anhydrous Na₂SO₄, filtered, and concentrated. The crude product was purified by flash silica gel chromatography (gradient: 0˜10% THF in petroleum ether) to give methyl 3-[[tert-butyl(diphenyl)silyl]oxymethyl]-1,2,3,5,6,7-hexahydropyrrolizine-8-carboxylate (24.3 g, 39.7 mmol, 35% yield) as a colorless oil (LC/MS (ESI) m/z: 438.2 [M+H]⁺), and methyl 3-[[tert-butyl(diphenyl)silyl]oxymethyl]-1,2,3,5,6,7-hexahydropyrrolizine-8-carboxylate (20.8 g, 43.0 mmol, 38% yield) as a colorless oil (LC/MS (ESI) m/z: 438.3 [M+H]⁺).

Step 6: Preparation of methyl (3R,8R)-3-[[tert-butyl(diphenyl)silyl]oxymethyl]-1,2,3,5,6,7-hexahydropyrrolizine-8-carboxylate and methyl (3S,8S)-3-[[tert-butyl(diphenyl)silyl]oxymethyl]-1,2,3,5,6,7-hexahydropyrrolizine-8-carboxylate

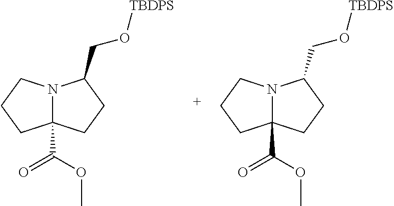

Methyl 3-[[tert-butyl(diphenyl)silyl]oxymethyl]-1,2,3,5,6,7-hexahydropyrrolizine-8-carboxylate (20.8 g, 47.5 mmol, 1 eq) was separated by SFC (column: REGIS (s,s) WHELK-O1 (250 mm*50 mm, 10 um); mobile phase: [0.1% NH₄OH EtOH]; B %: 25%, FlowRate: 140 mL/min). The pure fractions were combined and concentrated under reduced pressure to afford methyl (3R,8R)-3-[[tert-butyl(diphenyl)silyl]oxymethyl]-1,2,3,5,6,7-hexahydropyrrolizine-8-carboxylate (9.78 g, 22.35 mmol, 47% yield) as a colorless oil (LC/MS (ESI) m/z: 438.2 [M+H]⁺), and methyl (3S,8S)-3-[[tert-butyl(diphenyl)silyl]oxymethyl]-1,2,3,5,6,7-hexahydropyrrolizine-8-carboxylate (8.81 g, 20.1 mmol, 42.36% yield) as a colorless oil (LC/MS (ESI) m/z: 438.3 [M+H]⁺).

Step 7: Preparation of [(3S,8S)-3-[[tert-butyl(diphenyl)silyl]oxymethyl]-1,2,3,5,6,7-hexahydropyrrolizin-8-yl]methanol

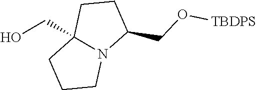

To a solution of methyl (3S,8S)-3-[[tert-butyl(diphenyl)silyl]oxymethyl]-1,2,3,5,6,7-hexahydropyrrolizine-8-carboxylate (1.57 g, 3.59 mmol, 1 eq) in THF (20 mL) at 0° C. was added LiAlH₄ (163 mg, 4.30 mmol, 1.2 eq), and the reaction mixture was stirred at 0° C. under N₂ for 1 hour. The reaction mixture was quenched by sequential addition of water (200 uL), 15% aq. NaOH (200 uL), and water (600 uL), then diluted with EtOAc (30 mL). The resulting suspension was dried over anhydrous Na₂SO₄, filtered and, concentrated. The crude product was purified by flash silica gel chromatography (gradient: 0˜10% CH₃OH in CH₂Cl₂) to give [(3S,8S)-3-[[tert-butyl(diphenyl)silyl]oxymethyl]-1,2,3,5,6,7-hexahydropyrrolizin-8-yl]methanol (911 mg, 2.22 mmol, 62% yield) as a yellow oil. LC/MS (ESI) m/z: 410.3 [M+H]⁺.

Step 8: Preparation of tert-butyl 3-[[2-[[(3S,8S)-3-[[tert-butyl(diphenyl)silyl]oxymethyl]-1,2,3,5,6,7-hexahydropyrrolizin-8-yl]methoxy]-7-chloro-8-fluoro-pyrido[4,3-d]pyrimidin-4-yl]-3,8-diazabicyclo[3.2.1]octane-8-carboxylate

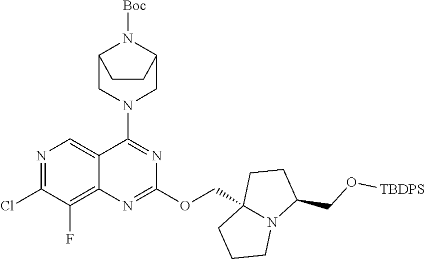

To a solution of [(3S,8S)-3-[[tert-butyl(diphenyl)silyl]oxymethyl]-1,2,3,5,6,7-hexahydropyrrolizin-8-yl]methanol (911 mg, 2.22 mmol, 1 eq) and tert-butyl 3-(2,7-dichloro-8-fluoro-pyrido[4,3-d]pyrimidin-4-yl)-3,8-diazabicyclo[3.2.1]octane-8-carboxylate (951 mg, 2.22 mmol, 1 eq) in dioxane (30 mL) were added Cs₂CO₃ (868 mg, 2.66 mmol, 1.2 eq) and DABCO (75 mg, 0.67 mmol, 0.3 eq), and the reaction mixture was stirred at 25° C. under N₂ for 16 hours. Additional DABCO (25 mg, 0.22 mmol, 0.1 eq) was added, and the reaction mixture was stirred at 25° C. under N₂ for 16 hours. The reaction mixture was filtered, and the filtrate was concentrated. The crude product was purified by prep-HPLC (35-65% CH₃CN in water (0.225% formic acid)). The pure fractions were combined and dried by lyophilization to give tert-butyl 3-[[2-[[(3S,8S)-3-[[tert-butyl(diphenyl)silyl]oxymethyl]-1,2,3,5,6,7-hexahydropyrrolizin-8-yl]methoxy]-7-chloro-8-fluoro-pyrido[4,3-d]pyrimidin-4-yl]-3,8-diazabicyclo[3.2.1]octane-8-carboxylate (794 mg, 0.991 mmol, 45% yield) as a yellow solid. LC/MS (ESI) m/z: 801.2 [M+H]⁺.

Step 9: Preparation of tert-butyl 3-[[2-[[(3S,8S)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

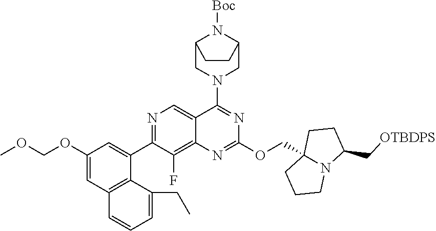

To a solution of tert-butyl 3-[[2-[[(3S,8S)-3-[[tert-butyl(diphenyl)silyl]oxymethyl]-1,2,3,5,6,7-hexahydropyrrolizin-8-yl]methoxy]-7-chloro-8-fluoro-pyrido[4,3-d]pyrimidin-4-yl]-3,8-diazabicyclo[3.2.1]octane-8-carboxylate (100 mg, 0.125 mmol, 1 eq) and 2-[8-ethyl-3-(methoxymethoxy)-1-naphthyl]-4,4,5,5-tetramethyl-1,3,2-dioxaborolane (51 mg, 0.15 mmol, 1.2 eq) in dioxane (2.5 mL) were added K₃PO₄ (1.5 M, 125 μL, 1.5 eq) and CataCXium® A Pd G3 (18 mg, 0.025 mmol, 0.2 eq), and the reaction mixture was stirred at 100° C. under N₂ (degassed under vacuum and purged with N₂ several times) for 12 hours. The reaction mixture was quenched by addition of water (10 mL) and extracted with EtOAc (3×10 mL). The combined organic extract was washed with brine (2×10 mL), dried over anhydrous Na₂SO₄, filtered, and concentrated. The crude product was purified by flash silica gel chromatography (gradient: 0˜20% THF in petroleum ether) to give tert-butyl 3-[[2-[[(3S,8S)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (162 mg, 0.165 mmol, 66% yield) as a yellow solid. LC/MS (ESI) m/z: 981.3 [M+H]⁺.

Step 10: Preparation of tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

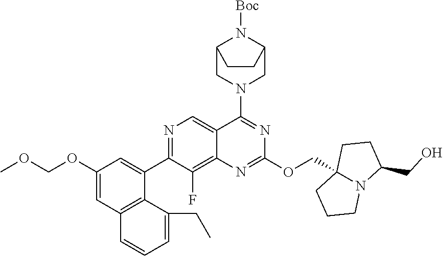 [SMILES: CCc1cccc2cc(OCOC)cc(-c3ncc4c(N5CC6CCC(C5)N6C(=O)OC(C)(C)C)nc(OC[C@@]56CCCN5[C@H](CO)CC6)nc4c3F)c12]

To a solution of tert-butyl 3-[[2-[[(3S,8S)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (162 mg, 0.165 mmol, 1 eq) in THF (2 mL) was added TBAF (1 M, 0.248 mL, 1.5 eq), and the reaction mixture was stirred at 25° C. for 3 hours. The reaction mixture was quenched by addition of water (10 mL) and extracted with EtOAc (3×10 mL). The combined organic extract was washed with brine (3×10 mL), dried over anhydrous Na₂SO₄, filtered, and concentrated. The crude product was purified by prep-TLC (acidic silica gel, EtOAc) to give tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (59 mg, 0.079 mmol, 48% yield) as a yellow solid. LC/MS (ESI) m/z: 743.1 [M+H]⁺.

Step 11: Preparation of tert-butyl 4-[[5-(1-methoxycarbonyl-2-methyl-propyl)isoxazol-3-yl]piperazine-1-carboxylate

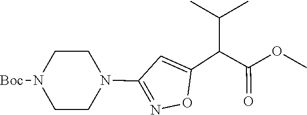 [SMILES: COC(=O)C(c1cc(N2CCN(C(=O)OC(C)(C)C)CC2)no1)C(C)C]

To a solution of methyl 3-methyl-2-[3-(1,1,2,2,3,3,4,4,4-nonafluorobutylsulfonyloxy)isoxazol-5-yl]butanoate (10 g, 20.78 mmol, 1.0 eq) in DMA (100 mL) were added tert-butyl piperazine-1-carboxylate (7.74 g, 41.56 mmol, 2.0 eq) and TEA (41.56 mmol, 5.8 mL, 2.0 eq), and the reaction mixture was stirred at 145° C. for 3 hours. The reaction mixture was quenched with water (100 mL) and extracted with EtOAc (3×100 mL). The combined organic extract was washed with brine (2×100 mL), dried over Na₂SO₄, filtered, and concentrated. The resulting residue was purified by flash chromatography on silica gel (gradient: 0˜6% THF in petroleum ether) to afford tert-butyl 4-[[5-(1-methoxycarbonyl-2-methyl-propyl)isoxazol-3-yl]piperazine-1-carboxylate (5.1 g, 13.88 mmol, 67% yield) as a colorless oil. LC/MS (ESI) m/z: 368.0 [M+H]⁺.

Step 12: Preparation of 2-[3-(4-tert-butoxycarbonylpiperazin-1-yl)isoxazol-5-yl]-3-methyl-butanoic acid

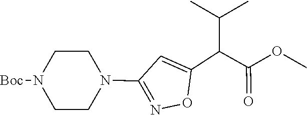 [SMILES: COC(=O)C(c1cc(N2CCN(C(=O)OC(C)(C)C)CC2)no1)C(C)C]

To a solution of tert-butyl 4-[[5-(1-methoxycarbonyl-2-methyl-propyl)isoxazol-3-yl]piperazine-1-carboxylate (5.49 g, 14.94 mmol, 1.0 eq) in CH₃OH (10 mL), THF (10 mL), and H₂O (10 mL) was added LiOH·H₂O (1.57 g, 37.35 mmol, 2.5 eq), and the reaction mixture was stirred at 25° C. for 1 hour. The mixture was concentrated, diluted with H₂O (10 mL), and the pH adjusted to pH ˜4 by addition of 2N aqueous HCl. The resulting mixture was extracted with EtOAc (3×20 mL), The combined organic extract was washed with brine (2×30 mL), dried over Na₂SO₄, filtered, and concentrated to give 2-[3-(4-tert-butoxycarbonylpiperazin-1-yl)isoxazol-5-yl]-3-methyl-butanoic acid (4.8 g, crude) as a white solid. LC/MS (ESI) m/z: 354.0 [M+H]⁺.

Step 13: Preparation of tert-butyl 4-[[5-[(1S)-1-[(2S,4R)-4-hydroxy-2-[[1-1-[4-(4-methylthiazol-5-yl)phenyl]ethyl]carbamoyl]pyrrolidine-1-carbonyl]-2-methyl-propyl]isoxazol-3-yl]piperazine-1-carboxylate

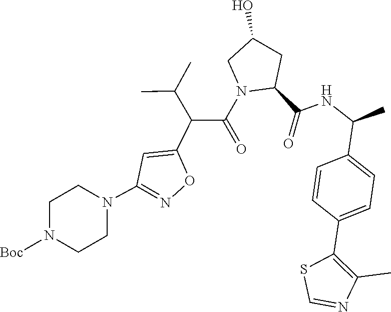 [SMILES: Cc1ncsc1-c1ccc([C@H](C)NC(=O)[C@@H]2C[C@@H](O)CN2C(=O)C(c2cc(N3CCN(C(=O)OC(C)(C)C)CC3)no2)C(C)C)cc1]

To a solution of 2-[3-(4-tert-butoxycarbonylpiperazin-1-yl)isoxazol-5-yl]-3-methyl-butanoic acid (4.8 g, 13.58 mmol, 1.0 eq) in CH₂Cl₂ (100 mL) were added DIEA (0.742 g/mL, 12.0 mL, 5.0 eq) and HATU (6.7 g, 17.65 mmol, 1.3 eq) followed by (2S,4R)-4-hydroxy-N-[(1S)-1-[4-(4-methylthiazol-5-yl)phenyl]ethyl]pyrrolidine-2-carboxamide (7.46 g, 13.59 mmol, 1.0 eq, HCl), and the reaction mixture was stirred at 25° C. for 0.5 hour. The reaction mixture was quenched with water (100 mL) and extracted with CH₂Cl₂ (3×100 mL). The combined organic extract was washed with brine (2×100 mL), dried over Na₂SO₄, filtered, and concentrated. The resulting residue was purified by flash chromatography on silica gel (gradient: 0˜70% THF in petroleum ether) to afford tert-butyl 4-[[5-[(1S)-1-[(2S,4R)-4-hydroxy-2-[[1-1-[4-(4-methylthiazol-5-yl)phenyl]ethyl]carbamoyl]pyrrolidine-1-carbonyl]-2-methyl-propyl]isoxazol-3-yl]piperazine-1-carboxylate (6.5 g, 8.5 mmol, 60% yield) as a white solid. LC/MS (ESI) m/z: 667.3 [M+H]⁺.

Step 14: Preparation of tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

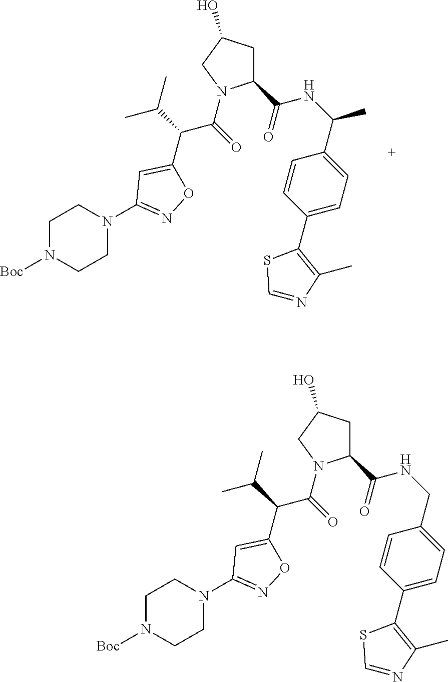 [SMILES: Cc1ncsc1-c1ccc([C@H](C)NC(=O)[C@@H]2C[C@@H](O)CN2C(=O)[C@H](c2cc(N3CCN(C(=O)OC(C)(C)C)CC3)no2)C(C)C)cc1 ; Cc1ncsc1-c1ccc(CNC(=O)[C@@H]2C[C@@H](O)CN2C(=O)[C@@H](c2cc(N3CCN(C(=O)OC(C)(C)C)CC3)no2)C(C)C)cc1]

The racemic product was purified by chiral-SFC (column: DAICEL CHIRALPAK AD (250 mm*50 mm, 10 um); mobile phase: [0.1% NH₄OH IPA]; B %: 45%, 15 min). The pure fractions were collected and concentrated under reduced pressure to afford tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (3.28 g, 4.92 mmol, 36% yield) as a white solid (LC/MS (ESI) m/z: 667.2 [M+H]⁺). The pure fractions was lyophilized to afford tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (1.87 g, 2.80 mmol, 21% yield) as a white solid (LC/MS (ESI) m/z: 667.3 [M+H]⁺).

Step 15: Preparation of (2S,4R)-4-hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide

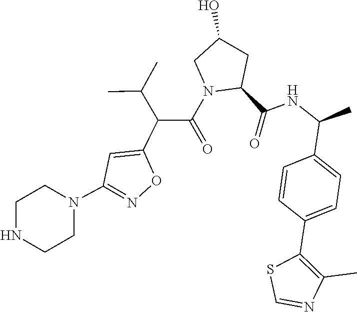 [SMILES: Cc1ncsc1-c1ccc([C@H](C)NC(=O)[C@@H]2C[C@@H](O)CN2C(=O)C(c2cc(N3CCNCC3)no2)C(C)C)cc1]

To a solution of tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (500.00 mg, 0.75 mmol, 1.0 eq) in CH₂Cl₂ (5 mL) was added HCl in dioxane (4 M, 2.62 mL, 14.0 eq), and the reaction mixture was stirred at 25° C. for 1 hour. The mixture was concentrated, diluted with H₂O (5 mL), and then the pH adjusted to pH ˜8 by addition of saturated aqueous NaHCO₃. The suspension was extracted with CH₂Cl₂/CH₃OH (3×20 mL, V/V=10/1), and the combined organic extract was dried over Na₂SO₄, filtered, and concentrated to afford (2S,4R)-4-hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (420 mg, crude) as a white solid. LC/MS (ESI) m/z: 567.3 [M+H]⁺.

Step 16: Preparation of tert-butyl 3-[7-[8-ethyl-3-(methoxymethoxy)-1-naphthyl]-8-fluoro-2-[[(3S,8S)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

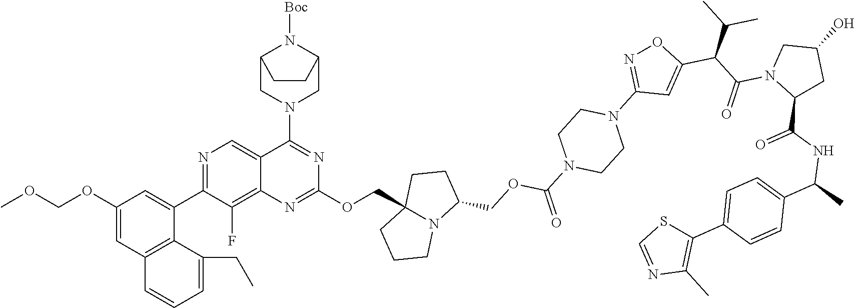 [SMILES: CCc1cccc2cc(OCOC)cc(-c3ncc4c(N5CC6CCC(C5)N6C(=O)OC(C)(C)C)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (50 mg, 0.067 mmol, 1 eq) in THF (2 mL) were added TEA (20 mg, 0.20 mmol, 3 eq), DMAP (0.8 mg, 0.007 mmol, 0.1 eq), and (4-nitrophenyl) carbonochloridate (27 mg, 0.13 mmol, 2 eq), and the reaction mixture was stirred at 25° C. under N₂ for 16 hours. (2S,4R)-4-Hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (50 mg, 0.088 mmol, 1.3 eq) was then added, and the reaction mixture was stirred at 25° C. under N₂ for 1 hour. The reaction mixture was quenched by addition of water (10 mL) and extracted with EtOAc (3×10 mL). The combined organic extract was washed with brine (2×10 mL), dried over anhydrous Na₂SO₄, filtered, and concentrated. The crude product was purified by prep-TLC (acidic silica gel, CH₃OH/CH₂Cl₂=1:10, Rf=0.47) to give tert-butyl 3-[7-[8-ethyl-3-(methoxymethoxy)-1-naphthyl]-8-fluoro-2-[[(3S,8S)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (49 mg, 0.037 mmol, 55% yield) as a yellow solid. LC/MS (ESI) m/z: 1335.3 [M+H]⁺.

Step 17: Preparation of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl-4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

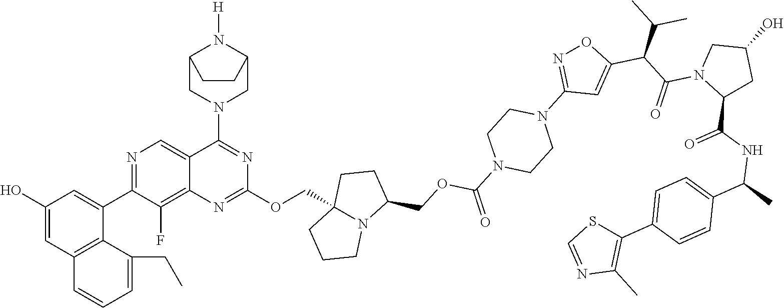 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

A solution of tert-butyl 3-[7-[8-ethyl-3-(methoxymethoxy)-1-naphthyl]-8-fluoro-2-[[(3S,8S)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (49 mg, 0.037 mmol, 1 eq) in HCOOH (3 mL) was stirred at 25° C. for 70 minutes. The reaction mixture was concentrated under reduced pressure, and the crude product was purified by prep-HPLC (0-50% CH₃CN in water (0.225% formic acid)). The pure fractions were combined and dried by lyophilization to give [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl-4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (24.6 mg, 19.88 umol, 54% yield, formic acid salt) as a white solid. LC/MS (ESI) m/z: 1191.5 [M+H]⁺. ¹H NMR (400 MHz, CD₃OD) δ 9.16-9.09 (m, 1H), 8.90 (s, 1H), 8.50 (s, 1H), 7.65 (d, J=8.4 Hz, 1H), 7.50-7.34 (m, 5H), 7.33-7.28 (m, 1H), 7.21-7.15 (m, 1H), 7.05-6.99 (m, 1H), 6.18-6.06 (m, 1H), 5.09-5.01 (m, 1H), 4.78-4.74 (m, 1H), 4.69 (d, J=12.0 Hz, 2H), 4.57-4.49 (m, 2H), 4.48-4.35 (m, 2H), 4.25-4.10 (m, 1H), 3.93-3.74 (m, 5H), 3.71-3.37 (m, 8H), 3.28-3.17 (m, 4H), 2.54-2.47 (m, 3H), 2.46-1.82 (m, 18H), 1.64-1.49 (m, 3H), 1.11-1.03 (m, 3H), 0.96-0.83 (m, 6H).

Exemplary Synthesis of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl-4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 4)

Step 1: Preparation of (2S,4R)-4-hydroxy-1-[(2S)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide

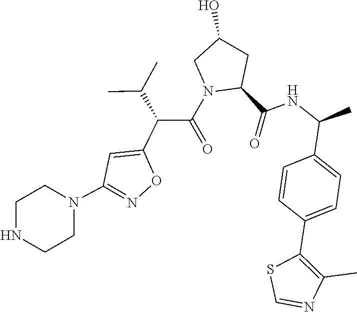 [SMILES: Cc1ncsc1-c1ccc([C@H](C)NC(=O)[C@@H]2C[C@@H](O)CN2C(=O)[C@H](c2cc(N3CCNCC3)no2)C(C)C)cc1]

To a solution of tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (500 mg, 749.82 umol, 1.0 eq) in CH₂Cl₂ (5 mL) was added HCl in dioxane (4 M, 2.62 mL, 14.0 eq), and the reaction mixture was stirred at 25° C. for 1 hour. The mixture was concentrated, diluted with H₂O (5 mL), and the pH adjusted to pH ˜8 by addition of saturated aqueous NaHCO₃. The suspension was extracted with CH₂Cl/CH₃OH (3×20 mL, V/V=10/1), and the combined organic extract was dried over Na₂SO₄, filtered, and concentrated to afford (2S,4R)-4-hydroxy-1-[(2S)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (420 mg, crude) as a white solid. LC/MS (ESI) m/z: 567.2 [M+H]⁺.

Step 2: Preparation of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl-4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

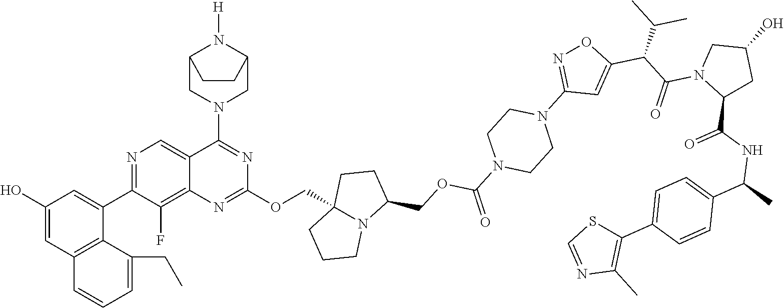 [SMILES: CCc1cccc2cc(O)cc(-c3ncc4c(N5CC6CCC(C5)N6)nc(OC[C@@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

This compound was prepared in an analogous manner to Compound 5 starting from tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2S)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide. (white solid, formic acid salt). LC/MS (ESI) m/z: 1191.6 [M+H]⁺. ¹H NMR (400 MHz, CD₃OD) δ 9.12-9.05 (m, 1H), 8.91-8.83 (m, 1H), 8.52 (s, 1H), 7.62 (d, J=8.4 Hz, 1H), 7.50-7.31 (m, 5H), 7.28 (t, J=2.4 Hz, 1H), 7.15 (d, J=7.2 Hz, 1H), 7.03-6.96 (m, 1H), 6.17-6.07 (m, 1H), 4.97 (q, J=6.8 Hz, 1H), 4.73-4.59 (m, 4H), 4.53-4.45 (m, 2H), 4.44-4.32 (m, 2H), 4.16-3.98 (m, 1H), 3.85-3.63 (m, 7H), 3.62-3.38 (m, 5H), 3.26-3.07 (m, 5H), 2.50-2.43 (m, 3H), 2.42-2.19 (m, 6H), 2.17-1.77 (m, 11H), 1.61-1.43 (m, 3H), 1.05 (d, J=6.5 Hz, 3H), 0.93-0.80 (m, 6H).

Exemplary Synthesis of [(3R,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 7)

Step 1: Preparation of [(3R,8R)-3-[[tert-butyl(diphenyl)silyl]oxymethyl]-1,2,3,5,6,7-hexahydropyrrolizin-8-yl]methanol

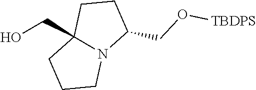

To a solution of methyl (3R,8R)-3-[[tert-butyl(diphenyl)silyl]oxymethyl]-1,2,3,5,6,7-hexahydropyrrolizine-8-carboxylate (2.69 g, 6.15 mmol, 1 eq) in THF (30 mL) at 0° C. was added LiAlH₄ (280 mg, 7.38 mmol, 1.2 eq), and the reaction mixture was stirred at 0° C. under N₂ for 1 hour. The reaction mixture was quenched by sequential addition of water (300 uL), 15% aq. NaOH (300 uL), and water (900 uL), then diluted with EtOAc (30 mL). The resulting suspension was dried over anhydrous Na₂SO₄, filtered, and concentrated. The crude product was purified by flash silica gel chromatography (gradient: 0˜10% CH₃OH in CH₂Cl₂) to give [(3R,8R)-3-[[tert-butyl(diphenyl)silyl]oxymethyl]-1,2,3,5,6,7-hexahydropyrrolizin-8-yl]methanol (2.07 g, 5.05 mmol, 82% yield) as a yellow oil. LC/MS (ESI) m/z: 410.1 [M+H]⁺.

Step 2: Preparation of tert-butyl 3-[[2-[[(3R,8R)-3-[[tert-butyl(diphenyl)silyl]oxymethyl]-1,2,3,5,6,7-hexahydropyrrolizin-8-yl]methoxy]-7-chloro-8-fluoro-pyrido[4,3-d]pyrimidin-4-yl]-3,8-diazabicyclo[3.2.1]octane-8-carboxylate

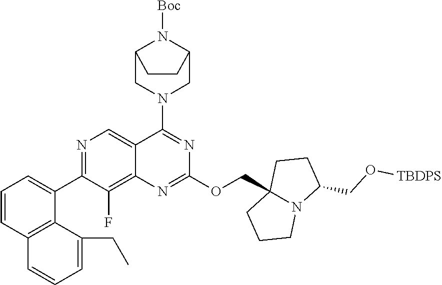

To a solution of [(3R,8R)-3-[[tert-butyl(diphenyl)silyl]oxymethyl]-1,2,3,5,6,7-hexahydropyrrolizin-8-yl]methanol (1.00 g, 2.44 mmol, 1 eq) and tert-butyl 3-(2,7-dichloro-8-fluoro-pyrido[4,3-d]pyrimidin-4-yl)-3,8-diazabicyclo[3.2.1]octane-8-carboxylate (1.05 g, 2.44 mmol, 1 eq) in dioxane (30 mL) were added Cs₂CO₃ (954 mg, 2.93 mmol, 1.2 eq) and DABCO (109 mg, 0.976 mmol, 0.4 eq), and the reaction mixture was stirred at 25° C. under N₂ for 40 hours. The reaction mixture was filtered, and the filtrate was concentrated. The crude product was purified by flash silica gel chromatography (gradient: 0˜20% THF in petroleum ether) to give tert-butyl 3-[[2-[[(3R,8R)-3-[[tert-butyl(diphenyl)silyl]oxymethyl]-1,2,3,5,6,7-hexahydropyrrolizin-8-yl]methoxy]-7-chloro-8-fluoro-pyrido[4,3-d]pyrimidin-4-yl]-3,8-diazabicyclo[3.2.1]octane-8-carboxylate (1.16 g, 1.13 mmol, 46% yield) as a yellow solid. LC/MS (ESI) m/z: 801.2 [M+H]⁺.

Step 3: Preparation of tert-butyl 3-[[2-[[(3R,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

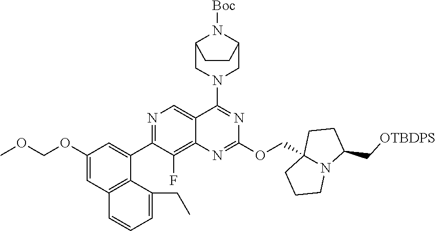

To a solution of tert-butyl 3-[[2-[[(3R,8R)-3-[[tert-butyl(diphenyl)silyl]oxymethyl]-1,2,3,5,6,7-hexahydropyrrolizin-8-yl]methoxy]-7-chloro-8-fluoro-pyrido[4,3-d]pyrimidin-4-yl]-3,8-diazabicyclo[3.2.1]octane-8-carboxylate (800 mg, 0.998 mmol, 1 eq) and 2-[8-ethyl-3-(methoxymethoxy)-1-naphthyl]-4,4,5,5-tetramethyl-1,3,2-dioxaborolane (376 mg, 1.10 mmol, 1.1 eq) in dioxane (20 mL) were added CataCXium® A Pd G3 (145 mg, 0.199 mmol, 0.2 eq) and K₂CO₃ (2.2 M, 2.04 mL, 4.5 eq), and the reaction mixture was stirred at 100° C. under N₂ (degassed under vacuum and purged with N₂ several times) for 12 hours. The reaction mixture was dried over anhydrous Na₂SO₄, filtered, and concentrated. The crude product was purified by flash silica gel chromatography (gradient: 0˜30% THF in petroleum ether) to give tert-butyl 3-[[2-[[(3R,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (695 mg, 0.446 mmol, 63% purity) as a yellow solid. LC/MS (ESI) m/z: 981.3 [M+H]⁺.

Step 4: Preparation of tert-butyl 3-[7-[8-ethyl-3-(methoxymethoxy)-1-naphthyl]-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

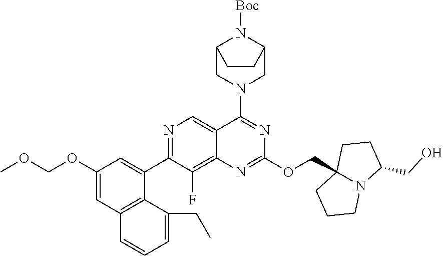 [SMILES: CCc1cccc2cc(OCOC)cc(-c3ncc4c(N5CC6CCC(C5)N6C(=O)OC(C)(C)C)nc(OC[C@]56CCCN5[C@@H](CO)CC6)nc4c3F)c12]

To a solution of tert-butyl 3-[[2-[[(3R,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (300 mg, 0.192 mmol, 63% purity, 1 eq) in THF (3 mL) was added TBAF (1 M, 458.59 uL, 1.5 eq), and the reaction mixture was stirred at 25° C. for 3 hours. The reaction mixture was quenched by addition of water (10 mL) and extracted with EtOAc (3×10 mL). The combined organic extract was washed with brine (3×10 mL), dried over anhydrous Na₂SO₄, filtered, and concentrated. The crude product was purified by prep-TLC (acidic silica gel, EtOAc) to give tert-butyl 3-[7-[8-ethyl-3-(methoxymethoxy)-1-naphthyl]-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (201 mg, 0.271 mmol, 44% yield) as a yellow solid. LC/MS (ESI) m/z: 743.1 [M+H]⁺.

Step 5: Preparation of tert-butyl 3-[7-[8-ethyl-3-(methoxymethoxy)-1-naphthyl]-8-fluoro-2-[[(3R,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

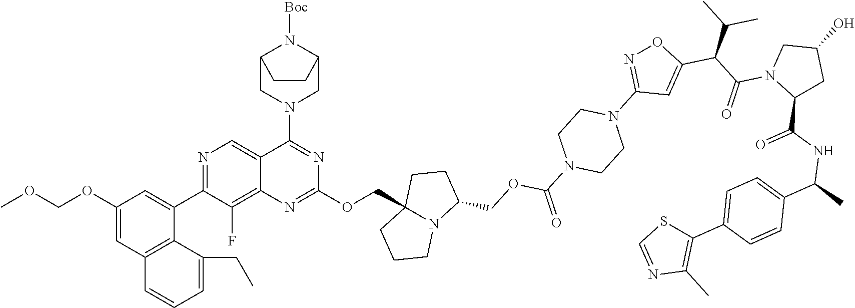 [SMILES: CCc1cccc2cc(OCOC)cc(-c3ncc4c(N5CC6CCC(C5)N6C(=O)OC(C)(C)C)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-[8-ethyl-3-(methoxymethoxy)-1-naphthyl]-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (40 mg, 0.054 mmol, 1 eq) in THF (2 mL) were added TEA (16 mg, 0.16 mmol, 3 eq), DMAP (0.6 mg, 0.005 mmol, 0.1 eq), and (4-nitrophenyl) carbonochloridate (22 mg, 0.11 mmol, 2 eq), and the reaction mixture was stirred at 25° C. under N₂ for 16 hours. (2S,4R)-4-Hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (34 mg, 0.059 mmol, 1.1 eq) was then added, and the reaction mixture was stirred at 25° C. under N₂ for 1 hour. The reaction mixture was quenched by addition of water (10 mL) and extracted with EtOAc (3×10 mL). The combined organic extract was washed with brine (3×10 mL), dried over anhydrous Na₂SO₄, filtered, and concentrated to give tert-butyl 3-[7-[8-ethyl-3-(methoxymethoxy)-1-naphthyl]-8-fluoro-2-[[(3R,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (70 mg, 0.052 mmol) as a yellow solid. LC/MS (ESI) m/z: 1335.6 [M+H].

Step 6: Preparation of [(3R,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

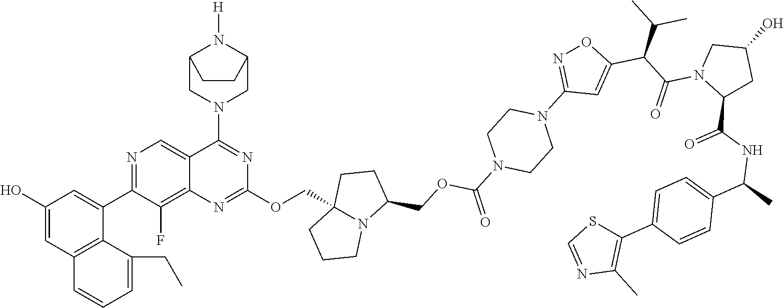 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

A solution of tert-butyl 3-[7-[8-ethyl-3-(methoxymethoxy)-1-naphthyl]-8-fluoro-2-[[(3R,8R)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (70 mg, 0.052 mmol, 1 eq) in formic acid (5 mL) was stirred at 25° C. for 1.5 hours. The reaction mixture was concentrated, and the crude product was purified by prep-HPLC (0-50% CH₃CN in water (0.225% formic acid)). The pure fractions were combined and dried by lyophilization to give [(3R,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (20.2 mg, 16.14 umol, 31% yield, formic acid) as a white solid. LC/MS (ESI) m/z: 1191.5 [M+H]⁺. ¹H NMR (400 MHz, CD₃OD) δ 9.12 (s, 1H), 8.88 (s, 1H), 8.43 (s, 2H), 7.63 (d, J=8.3 Hz, 1H), 7.48-7.32 (m, 5H), 7.31-7.27 (m, 1H), 7.19-7.13 (m, 1H), 7.03-6.95 (m, 1H), 6.15-6.06 (m, 1H), 5.03 (q, J=6.8 Hz, 1H), 4.76-4.68 (m, 3H), 4.58-4.48 (m, 2H), 4.46-4.35 (m, 2H), 4.28-4.16 (m, 1H), 4.03-3.95 (m, 2H), 3.93-3.73 (m, 3H), 3.68-3.48 (m, 7H), 3.43-3.34 (m, 1H), 3.27-3.17 (m, 4H), 2.49-2.46 (m, 3H), 2.45-1.91 (m, 18H), 1.60-1.48 (m, 3H), 1.05 (d, J=6.5 Hz, 3H), 0.94-0.83 (m, 6H).

Exemplary Synthesis of [(3R,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 6)

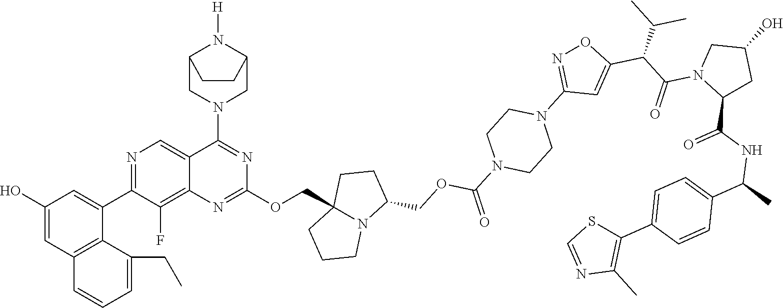 [SMILES: CCc1cccc2cc(O)cc(-c3ncc4c(N5CC6CCC(C5)N6)nc(OC[C@]56CCCN5[C@@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

This compound was prepared in an analogous manner to Compound 7 starting from tert-butyl 3-[7-[8-ethyl-3-(methoxymethoxy)-1-naphthyl]-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2S)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide. (white solid, formic acid salt). LC/MS (ESI) m/z: 1191.4 [M+H]⁺. ¹H NMR (400 MHz, CD₃OD) δ 9.07 (s, 1H), 8.92-8.86 (m, 1H), 8.56 (s, 1H), 7.67-7.59 (m, 1H), 7.52-7.33 (m, 5H), 7.31-7.25 (m, 1H), 7.20-7.11 (m, 1H), 7.05-6.97 (m, 1H), 6.21-6.10 (m, 1H), 5.01-4.98 (m, 1H), 4.72-4.67 (m, 1H), 4.62-4.56 (m, 2H), 4.54-4.22 (m, 5H), 3.89-3.40 (m, 12H), 3.29-2.92 (m, 6H), 2.52-2.46 (m, 3H), 2.46-1.71 (m, 17H), 1.63-1.45 (m, 3H), 1.10-0.97 (m, 3H), 0.96-0.82 (m, 6H).

Exemplary Synthesis of (2S,4R)-1-[(2R)-2-[3-[[1-[2-[4-(3,8-diazabicyclo[3.2.1]octan-3-yl)-8-fluoro-7-(5-methyl-1H-indazol-4-yl)pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 3)

Step 1: Preparation of tert-butyl 3-[[2-(2,2-dimethoxyethoxy)-8-fluoro-7-(5-methyl-1H-indazol-4-yl)pyrido[4,3-d]pyrimidin-4-yl]-3,8-diazabicyclo[3.2.1]octane-8-carboxylate

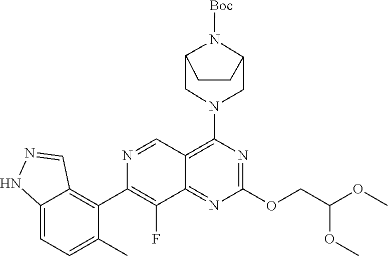 [SMILES: COC(COc1nc(N2CC3CCC(C2)N3C(=O)OC(C)(C)C)c2cnc(-c3c(C)ccc4[nH]ncc34)c(F)c2n1)OC]

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (500 mg, 1.00 mmol, 1.0 eq) in dioxane (15 mL) and H₂O (3 mL) were added (5-methyl-1H-indazol-4-yl) boronic acid (883 mg, 5.02 mmol, 5.0 eq), Cs₂CO₃ (818 mg, 2.51 mmol, 2.5 eq) and [1,1′-bis(di-tert-butylphosphino)ferrocene]dichloropalladium (II) (65 mg, 0.1 mmol, 0.1 eq), and the reaction mixture was degassed and purged with N₂ (3×), then stirred at 100° C. for 10 hours under N₂. The mixture was dried over anhydrous Na₂SO₄, filtered, and concentrated, and the resulting residue was purified by flash chromatography on silica gel (gradient: 0˜30% THF in petroleum ether) to afford tert-butyl3-[[2-(2,2-dimethoxyethoxy)-8-fluoro-7-(5-methyl-1H-indazol-4-yl)pyrido[4,3-d]pyrimidin-4-yl]-3,8-diazabicyclo[3.2.1]octane-8-carboxylate (590 mg, 834.84 umol, 83% yield) as a yellow solid. LC/MS (ESI) m/z: 594.3 [M+H]⁺.

Step 2: Preparation of tert-butyl 3-[8-fluoro-7-(5-methyl-1H-indazol-4-yl)-2-(2-oxoethoxy)pyrido[4,3-d]pyrimidin-4-yl]-3,8-diazabicyclo[3.2.1]octane-8-carboxylate

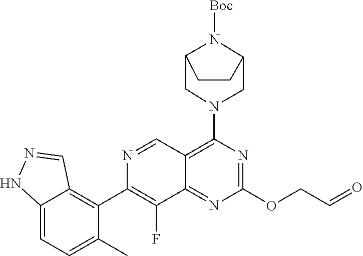 [SMILES: Cc1ccc2[nH]ncc2c1-c1ncc2c(N3CC4CCC(C3)N4C(=O)OC(C)(C)C)nc(OCC=O)nc2c1F]

To a solution of tert-butyl 3-[[2-(2,2-dimethoxyethoxy)-8-fluoro-7-(5-methyl-1H-indazol-4-yl)pyrido[4,3-d]pyrimidin-4-yl]-3,8-diazabicyclo[3.2.1]octane-8-carboxylate (200 mg, 0.33 mmol, 1.0 eq) in acetone (0.5 mL) was added HCl (12 M, 505 μL, 18.0 eq), and the reaction mixture was stirred at 25° C. for 20 minutes. The reaction mixture was poured onto a solution of NaHCO₃ (969 mg, 11.53 mmol, 35 eq) in water (2 mL)/THF (2 mL). Boc₂₀ (0.075 mmol, 86 μL, 1.1 eq) was then added, and the resulting mixture was stirred at 25° C. for 1 hour. The reaction mixture was diluted with water (50 mL) and extracted with CH₂Cl₂ (3×50 mL), the combined organic layer was dried over anhydrous Na₂SO₄, filtered, and concentrated. The resulting residue was purified by flash chromatography on silica gel (gradient: 0˜100% EtOAc in petroleum ether, then 0-10% CH₃OH in CH₂Cl₂) to afford tert-butyl 3-[8-fluoro-7-(5-methyl-1H-indazol-4-yl)-2-(2-oxoethoxy)pyrido[4,3-d]pyrimidin-4-yl]-3,8-diazabicyclo[3.2.1]octane-8-carboxylate (240 mg, 0.44 mmol, 65% yield) as a yellow solid. LC/MS (ESI) m/z: 548.1 [M+H]⁺.

Step 3: Preparation of (2S,4R)-1-[(2R)-2-[3-[[1-[2-[4-(3,8-diazabicyclo[3.2.1]octan-3-yl)-8-fluoro-7-(5-methyl-1H-indazol-4-yl)pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

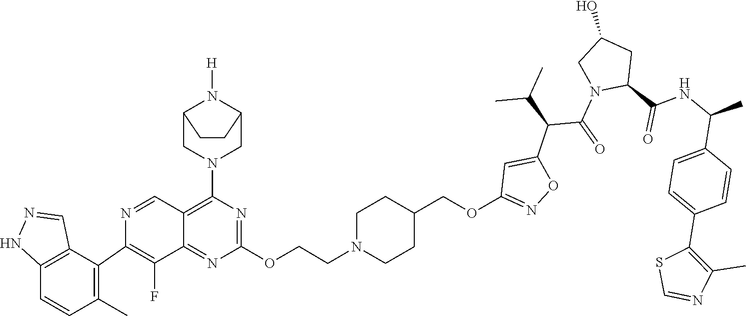 [SMILES: Cc1ccc2[nH]ncc2c1-c1ncc2c(N3CC4CCC(C3)N4)nc(OCCN3CCC(COc4cc([C@H](C(=O)N5C[C@H](O)C[C@H]5C(=O)N[C@@H](C)c5ccc(-c6scnc6C)cc5)C(C)C)on4)CC3)nc2c1F]

This compound was prepared in an analogous manner to Compound 20 starting from tert-butyl 3-[8-fluoro-7-(5-methyl-1H-indazol-4-yl)-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide. (white solid, formic acid salt). LC/MS (ESI) m/z: 1027.4 [M+H]⁺. ¹H NMR (400 MHz, CD₃OD) δ 9.18 (s, 1H), 8.87 (s, 1H), 8.46 (s, 1H), 7.70 (s, 1H), 7.64-7.58 (m, 1H), 7.48-7.33 (m, 5H), 6.01-5.92 (m, 1H), 5.09-4.97 (m, 1H), 4.83-4.72 (m, 6H), 4.51 (t, J=8.0 Hz, 1H), 4.46-4.36 (m, 1H), 4.13-4.06 (m, 2H), 4.00 (s, 2H), 3.90-3.79 (m, 3H), 3.77-3.34 (m, 5H), 2.82-2.69 (m, 2H), 2.49-2.45 (m, 3H), 2.35 (s, 3H), 2.24-2.12 (m, 1H), 2.08-1.90 (m, 8H), 1.67-1.49 (m, 5H), 1.05 (d, J=6.4 Hz, 3H), 0.93-0.84 (m, 3H).

Exemplary Synthesis of (2S,4R)-1-[(2S)-2-(2-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide (Compound 2)

Step 1: Preparation of tert-butyl 3-[7-chloro-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

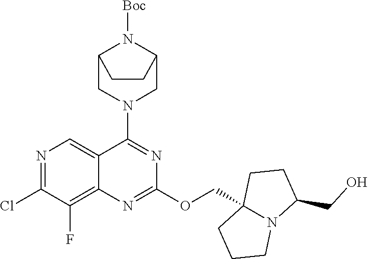 [SMILES: CC(C)(C)OC(=O)N1C2CCC1CN(c1nc(OC[C@@]34CCCN3[C@H](CO)CC4)nc3c(F)c(Cl)ncc13)C2]

To a solution of tert-butyl 3-[[2-[[(3S,8S)-3-[[tert-butyl(diphenyl)silyl]oxymethyl]-1,2,3,5,6,7-hexahydropyrrolizin-8-yl]methoxy]-7-chloro-8-fluoro-pyrido[4,3-d]pyrimidin-4-yl]-3,8-diazabicyclo[3.2.1]octane-8-carboxylate (1.20 g, 1.50 mmol, 1 eq) in THF (10 mL) was added TBAF (1 M, 2.25 mL, 1.5 eq), and the reaction mixture was stirred at 25° C. for 16 hours. The reaction mixture was concentrated, and the crude product was purified by flash chromatography on silica gel (gradient: 0˜30% THF in CH₂Cl₂) to give tert-butyl 3-[7-chloro-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (747 mg, 1.33 mmol, 89% yield) as a yellow solid. LC/MS (ESI) m/z: 563.1 [M+H]⁺.

Step 2: Preparation of tert-butyl 3-[7-chloro-2-[[(3S,8S)-3-[(2-ethoxy-2-oxo-ethoxy)methyl]-1,2,3,5,6,7-hexahydropyrrolizin-8-yl]methoxy]-8-fluoro-pyrido[4,3-d]pyrimidin-4-yl]-3,8-diazabicyclo[3.2.1]octane-8-carboxylate

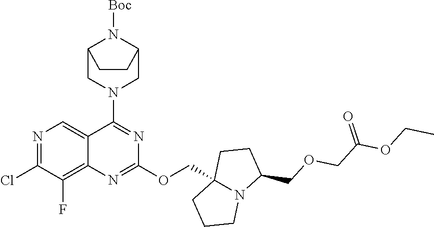 [SMILES: CCOC(=O)COC[C@@H]1CC[C@]2(COc3nc(N4CC5CCC(C4)N5C(=O)OC(C)(C)C)c4cnc(Cl)c(F)c4n3)CCCN12]

To a solution of tert-butyl 3-[7-chloro-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (747 mg, 1.33 mmol, 1 eq) in CH₂Cl₂ (10 mL) at 0° C. were added Rh₂(OAc)₄ (59 mg, 0.13 mmol, 0.1 eq) and ethyl 2-diazoacetate (303 mg, 2.65 mmol, 2 eq), and the reaction mixture was stirred at 25° C. under N₂ for 16 hours. Additional Rh₂(OAc)₄ (59 mg, 0.13 mmol, 0.1 eq) and ethyl 2-diazoacetate (303 mg, 2.65 mmol, 2 eq) were added at 0° C., and the reaction mixture was stirred at 25° C. under N₂ for 20 hours. The reaction mixture was filtered, and additional Rh₂(OAc)₄ (59 mg, 0.13 mmol, 0.1 eq) and ethyl 2-diazoacetate (303 mg, 2.65 mmol, 2 eq) were added at 0° C., and the reaction mixture was stirred at 25° C. under N₂ for 20 hours. The reaction mixture was filtered, and the filtrate was concentrated under reduced pressure. The crude product was purified flash chromatography on silica gel (gradient: 0˜20% THF in CH₂Cl₂) to give tert-butyl 3-[7-chloro-2-[[(3S,8S)-3-[(2-ethoxy-2-oxo-ethoxy)methyl]-1,2,3,5,6,7-hexahydropyrrolizin-8-yl]methoxy]-8-fluoro-pyrido[4,3-d]pyrimidin-4-yl]-3,8-diazabicyclo[3.2.1]octane-8-carboxylate (270 mg, 0.416 mmol, 31% yield) as a yellow gum. LC/MS (ESI) m/z: 649.1 [M+H]⁺.

Step 3: Preparation of tert-butyl 3-[[2-[[(3S,8S)-3-[(2-ethoxy-2-oxo-ethoxy)methyl]-1,2,3,5,6,7-hexahydropyrrolizin-8-yl]methoxy]-8-fluoro-7-[3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate

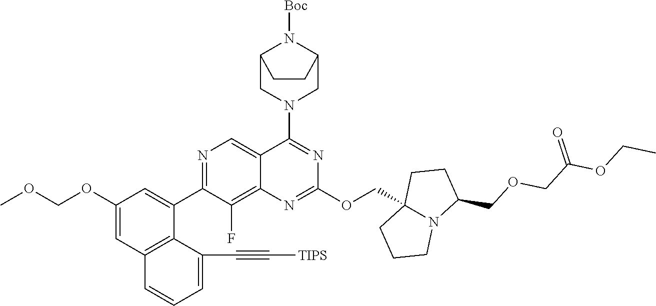

To a solution of tert-butyl 3-[7-chloro-2-[[(3S,8S)-3-[(2-ethoxy-2-oxo-ethoxy)methyl]-1,2,3,5,6,7-hexahydropyrrolizin-8-yl]methoxy]-8-fluoro-pyrido[4,3-d]pyrimidin-4-yl]-3,8-diazabicyclo[3.2.1]octane-8-carboxylate (270 mg, 0.416 mmol, 1 eq) and triisopropyl-[2-[6-(methoxymethoxy)-8-(4,4,5,5-tetramethyl-1,3,2-dioxaborolan-2-yl)-1-naphthyl]ethynyl]silane (228 mg, 0.460 mol, 1.4 eq) in dioxane (6 mL) were added CataCXium® A Pd G3 (48 mg, 0.066 mmol, 0.2 eq) and K₃PO₄ (1 M, 0.657 mL, 2 eq), and the reaction mixture was stirred at 100° C. under N₂ (degassed under vacuum and purged with N₂ several times) for 10 hours. The reaction mixture was diluted with CH₂Cl₂ (15 mL) and then dried over anhydrous Na₂SO₄, filtered, and concentrated. The crude product was purified by flash chromatography on silica gel (gradient 0˜8% CH₃OH in CH₂Cl₂) to give tert-butyl 3-[[2-[[(3S,8S)-3-[(2-ethoxy-2-oxo-ethoxy)methyl]-1,2,3,5,6,7-hexahydropyrrolizin-8-yl]methoxy]-8-fluoro-7-[3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (121 mg, 0.123 mmol, 38% yield) as yellow gum. LC/MS (ESI) m/z: 981.3 [M+H]⁺

Step 4: Preparation of tert-butyl 3-[[2-[[(3S,8S)-3-[(2-ethoxy-2-oxo-ethoxy)methyl]-1,2,3,5,6,7-hexahydropyrrolizin-8-yl]methoxy]-7-[8-ethynyl-3-(methoxymethoxy)-1-naphthyl]-8-fluoro-pyrido[4,3-d]pyrimidin-4-yl]-3,8-diazabicyclo[3.2.1]octane-8-carboxylate

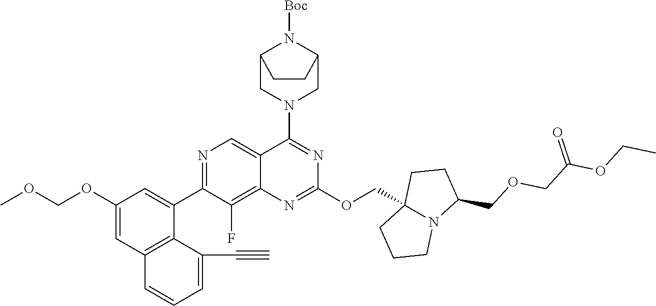 [SMILES: C#Cc1cccc2cc(OCOC)cc(-c3ncc4c(N5CC6CCC(C5)N6C(=O)OC(C)(C)C)nc(OC[C@@]56CCCN5[C@H](COCC(=O)OCC)CC6)nc4c3F)c12]

To a solution of tert-butyl 3-[[2-[[(3S,8S)-3-[(2-ethoxy-2-oxo-ethoxy)methyl]-1,2,3,5,6,7-hexahydropyrrolizin-8-yl]methoxy]-8-fluoro-7-[3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (121 mg, 0.123 mmol, 1 eq) in DMF (1 mL) was added CsF (131 mg, 0.863 mmol, 7 eq), and the reaction mixture was stirred at 25° C. for 1 hour. The reaction mixture was diluted CH₂Cl₂ (15 mL) and filtered. The filtrate was concentrated under reduced to give tert-butyl 3-[[2-[[(3S,8S)-3-[(2-ethoxy-2-oxo-ethoxy)methyl]-1,2,3,5,6,7-hexahydropyrrolizin-8-yl]methoxy]-7-[8-ethynyl-3-(methoxymethoxy)-1-naphthyl]-8-fluoro-pyrido[4,3-d]pyrimidin-4-yl]-3,8-diazabicyclo[3.2.1]octane-8-carboxylate (100 mg, 0.121 mmol) as a yellow oil. LC/MS (ESI) m/z: 825.3 [M+H]⁺.

Step 5: Preparation of tert-butyl 3-[[2-[[(3S,8S)-3-[(2-ethoxy-2-oxo-ethoxy)methyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

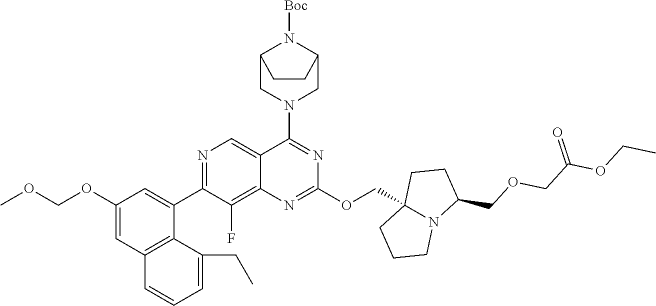 [SMILES: CCOC(=O)COC[C@@H]1CC[C@]2(COc3nc(N4CC5CCC(C4)N5C(=O)OC(C)(C)C)c4cnc(-c5cc(OCOC)cc6cccc(CC)c56)c(F)c4n3)CCCN12]

To a solution of tert-butyl 3-[[2-[[(3S,8S)-3-[(2-ethoxy-2-oxo-ethoxy)methyl]-1,2,3,5,6,7-hexahydropyrrolizin-8-yl]methoxy]-7-[8-ethynyl-3-(methoxymethoxy)-1-naphthyl]-8-fluoro-pyrido[4,3-d]pyrimidin-4-yl]-3,8-diazabicyclo[3.2.1]octane-8-carboxylate (100 mg, 0.121 mmol, 1 eq) in CH₃OH (3 mL) was added Pd/C (20 mg, 10% purity), and the reaction mixture was stirred at 25° C. under H₂ (15 psi) (degassed under vacuum and purged with H₂ several times) for 1 hour. The reaction mixture was filtered, and the filtrate was concentrated under reduced pressure to give tert-butyl 3-[[2-[[(3S,8S)-3-[(2-ethoxy-2-oxo-ethoxy)methyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (100 mg, 0.120 mmol) as a yellow solid. LC/MS (ESI) m/z: 829.5 [M+H]⁺.

Step 6: Preparation of 2-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]acetic acid

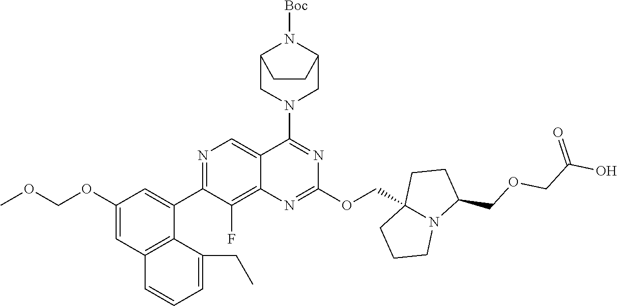 [SMILES: CCc1cccc2cc(OCOC)cc(-c3ncc4c(N5CC6CCC(C5)N6C(=O)OC(C)(C)C)nc(OC[C@@]56CCCN5[C@H](COCC(=O)O)CC6)nc4c3F)c12]

To a solution of tert-butyl 3-[[2-[[(3S,8S)-3-[(2-ethoxy-2-oxo-ethoxy)methyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (100 mg, 0.120 mmol, 1 eq) in THF (1 mL) and H₂O (1 mL) was added LiOH·H₂O (20 mg, 0.48 mmol, 4 eq), and the reaction mixture was stirred at 25° C. under N₂ for 2 hours. The reaction mixture was concentrated under reduced pressure to remove THE, and the resulting residue was diluted with water (10 mL). The pH of the resulting aqueous mixture was adjusted to pH=5˜6 by addition of 2N aqueous HCl. Lyophilization afforded 2-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]acetic acid (95 mg, crude) as a yellow solid. LC/MS (ESI) m/z: 801.2 [M+H]⁺.

Step 7: Preparation of tert-butyl 3-[7-[8-ethyl-3-(methoxymethoxy)-1-naphthyl]-8-fluoro-2-[[(3S,8S)-3-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

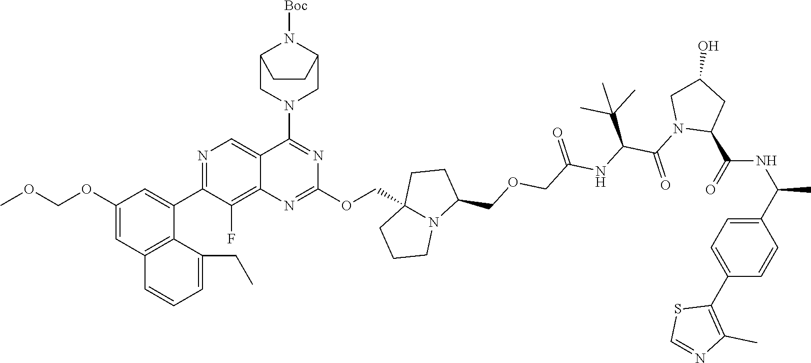 [SMILES: CCc1cccc2cc(OCOC)cc(-c3ncc4c(N5CC6CCC(C5)N6C(=O)OC(C)(C)C)nc(OC[C@@]56CCCN5[C@H](COCC(=O)N[C@H](C(=O)N5C[C@H](O)C[C@H]5C(=O)N[C@@H](C)c5ccc(-c7scnc7C)cc5)C(C)(C)C)CC6)nc4c3F)c12]

To a solution of 2-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]acetic acid (95 mg, 0.119 mmol, 1 eq) in DMF (2 mL) were added DIEA (77 mg, 0.59 mmol, 5 eq), HATU (68 mg, 0.18 mmol, 1.5 eq), and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (57 mg, 0.119 mmol, 1 eq, HCl), and the reaction mixture was stirred at 25° C. under N₂ for 4 hours. The reaction mixture was quenched by addition of water (15 mL) and extracted with CH₂Cl₂ (3×15 mL). The combined organic extract was washed with brine (3×15 mL), dried over anhydrous Na₂SO₄, filtered, and concentrated. The crude product was purified by prep-TLC (acidic silica gel, CH₃OH/CH₂Cl₂=1:10, Rf=0.44) to give tert-butyl 3-[7-[8-ethyl-3-(methoxymethoxy)-1-naphthyl]-8-fluoro-2-[[(3S,8S)-3-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (89 mg, 0.058 mmol, 49% yield) as a yellow solid. LC/MS (ESI) m/z: 1227.6 [M+H].

Step 8: Preparation of (2S,4R)-1-[(2S)-2-(2-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide

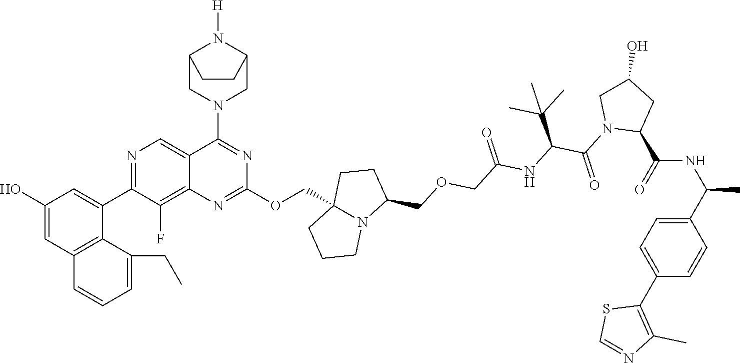 [SMILES: CCc1cccc2cc(O)cc(-c3ncc4c(N5CC6CCC(C5)N6)nc(OC[C@@]56CCCN5[C@H](COCC(=O)N[C@H](C(=O)N5C[C@H](O)C[C@H]5C(=O)N[C@@H](C)c5ccc(-c7scnc7C)cc5)C(C)(C)C)CC6)nc4c3F)c12]

A solution of tert-butyl 3-[7-[8-ethyl-3-(methoxymethoxy)-1-naphthyl]-8-fluoro-2-[[(3S,8S)-3-[[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethoxy]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (89 mg, 0.058 mmol, 80% purity, 1 eq) in HCOOH (5 mL) was stirred at 25° C. for 2.5 hours. The reaction mixture was concentrated under reduced pressure, and the crude product was purified by prep-HPLC (0-50% CH₃CN in water (0.225% formic acid). The pure fractions were combined and dried by lyophilization to give (2S,4R)-1-[(2S)-2-(2-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide (26.1 mg, 0.022 mmol, 38% yield, formic acid salt) as an off-white solid. LC/MS (ESI) m/z: 1083.5 [M+H]⁺. ¹H NMR (400 MHz, CD₃OD) δ 9.18-9.08 (m, 1H), 8.90-8.83 (m, 1H), 8.46 (s, 2H), 7.76-7.58 (m, 1H), 7.46-7.33 (m, 5H), 7.32-7.28 (m, 1H), 7.19-7.11 (m, 1H), 7.04-6.97 (m, 1H), 5.01-4.97 (m, 1H), 4.72-4.68 (m, 1H), 4.67-4.62 (m, 2H), 4.56-4.50 (m, 1H), 4.44-4.34 (m, 1H), 4.30-4.11 (m, 3H), 4.03-3.94 (m, 2H), 3.93-3.78 (m, 5H), 3.74-3.56 (m, 2H), 3.52-3.32 (m, 2H), 2.48-1.93 (m, 20H), 1.58-1.44 (m, 3H), 1.01 (s, 9H), 0.93-0.83 (m, 3H).

Exemplary Synthesis of (2S,4R)-1-[(2S)-2-[[2-[[(3R,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]acetyl]amino]-3,3-dimethyl-butanoyl]-4-hydroxy-N-[(1S)-1-[4-(4-methylthiazol-5-yl)phenyl]ethyl]pyrrolidine-2-carboxamide (Compound 1)

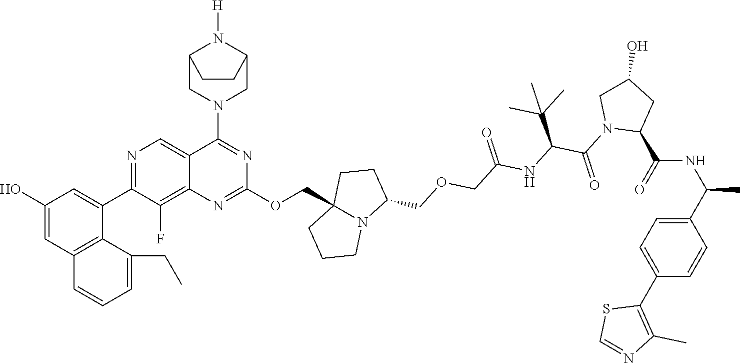 [SMILES: CCc1cccc2cc(O)cc(-c3ncc4c(N5CC6CCC(C5)N6)nc(OC[C@]56CCCN5[C@@H](COCC(=O)N[C@H](C(=O)N5C[C@H](O)C[C@H]5C(=O)N[C@@H](C)c5ccc(-c7scnc7C)cc5)C(C)(C)C)CC6)nc4c3F)c12]

This compound was prepared in an analogous manner to Compound 2 starting from tert-butyl 3-[[2-[[(3R,8R)-3-[[tert-butyl(diphenyl)silyl]oxymethyl]-1,2,3,5,6,7-hexahydropyrrolizin-8-yl]methoxy]-7-chloro-8-fluoro-pyrido[4,3-d]pyrimidin-4-yl]-3,8-diazabicyclo[3.2.1]octane-8-carboxylate. (white solid, formic acid salt). LC/MS (ESI) m/z: 1083.5 [M+H]⁺. ¹H NMR (400 MHz, CD₃OD) δ 9.17-9.08 (m, 1H), 8.91-8.82 (m, 1H), 8.44 (s, 1H), 7.63 (d, J=8.4 Hz, 1H), 7.46-7.32 (m, 5H), 7.30 (d, J=2.4 Hz, 1H), 7.16 (d, J=6.8 Hz, 1H), 7.01 (t, J=2.4 Hz, 1H), 4.99-4.96 (m, 1H), 4.75-4.69 (m, 2H), 4.64-4.60 (m, 1H), 4.57-4.50 (m, 1H), 4.45-4.35 (m, 1H), 4.31-4.08 (m, 3H), 4.06-3.96 (m, 2H), 3.94-3.77 (m, 5H), 3.76-3.58 (m, 2H), 3.51-3.34 (m, 2H), 2.49-1.89 (m, 20H), 1.57-1.43 (m, 3H), 1.06-0.97 (m, 9H), 0.93-0.83 (m, 3H).

Exemplary Synthesis of (2S,4R)-1-[(2R)-2-{3-[(1-{2-[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 48)

Step 1: Preparation of tert-butyl 3-[[2-(2,2-dimethoxyethoxy)-8-fluoro-7-[3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate

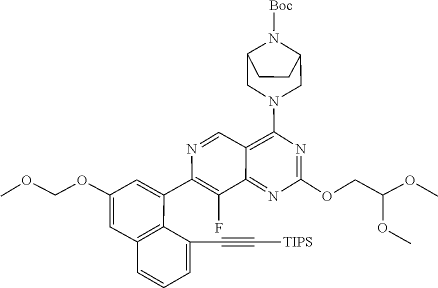

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (600 mg, 1.20 mmol, 1.0 eq), triisopropyl-[2-[6-(methoxymethoxy)-8-(4,4,5,5-tetramethyl-1,3,2-dioxaborolan-2-yl)-1-naphthyl]ethynyl]silane (834 mg, 1.69 mmol, 1.4 eq), and K₃PO₄ (767 mg, 3.61 mmol, 3.0 eq) in dioxane (10 mL) and H₂O (2 mL) was added [2-(2-aminophenyl)phenyl]palladium (1+); bis(1-adamantyl)-butyl-phosphane; methanesulfonate (175 mg, 0.24 mmol, 0.2 eq), and the reaction mixture was degassed and purged with N₂ (3×), then stirred at 100° C. under N₂ atmosphere for 10 hours. The mixture was dried over anhydrous sodium sulfate, filtered, and concentrated in vacuum. The resulting residue was purified by flash chromatography on SiO₂ (gradient: 0˜10% methanol in dichloromethane) to afford tert-butyl 3-[[2-(2,2-dimethoxyethoxy)-8-fluoro-7-[3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (870 mg, 0.987 mmol, 82% yield) as a yellow solid. LC/MS (ESI) m/z: 830.3 [M+H]⁺.

Step 2: Preparation of tert-butyl 3-[8-fluoro-7-[3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate

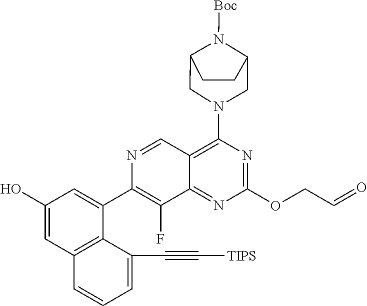

To a solution of tert-butyl 3-[[2-(2,2-dimethoxyethoxy)-8-fluoro-7-[3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (810 mg, 0.97 mmol, 1.0 eq) in acetone (1.5 mL) was added aqueous HCl (12 M, 1.5 mL, 18.53 eq), and the reaction mixture was stirred at 25° C. for 5 minutes. The reaction mixture was poured onto a solution of NaHCO₃ (2.8 g, 33.4 mmol, 1.30 mL, 34.0 eq) in water (10 mL)/THF (10 mL). Boc₂O (250 uL, 1.09 mmol, 1.11 eq) was then added, and the resulting mixture was stirred at 25° C. for 1 hour. The reaction mixture was diluted with water (50 mL) and extracted with CH₂Cl₂ (50 mL×3). The combined organic extract was dried over anhydrous Na₂SO₄, filtered, and concentrated under reduced pressure. The resulting residue was purified by flash chromatography on SiO₂ (gradient: 0˜100% Ethyl acetate in petroleum ether followed by 0˜10% methanol in dichloromethane) to afford tert-butyl 3-[8-fluoro-7-[3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (550 mg) as a yellow solid. LC/MS (ESI) m/z: 740.2 [M+H]⁺.

Step 3: Preparation of tert-butyl 3-[8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]-7-[3-hydroxy-8-(2-triisopropylsilylethynyl)-1-naphthyl]pyrido[4,3-d]pyrimidin-4-yl]-3,8-diazabicyclo[3.2.1]octane-8-carboxylate

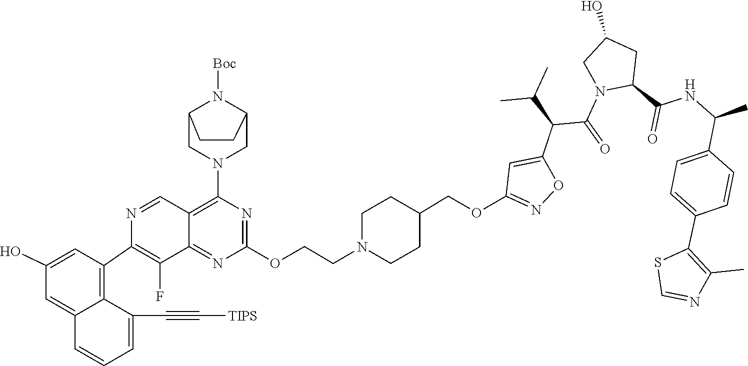

To a solution of tert-butyl 3-[8-fluoro-7-[3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (550 mg, 0.617 mmol, 83% purity, 1.0 eq) and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (367 mg, 0.617 mmol, 1.0 eq) in CH₂Cl₂ (5 mL) and isopropanol (5 mL) were added acetic acid (3.08 mmol, 176 μL, 5.0 eq) and 2-methylpyridine borane (330 mg, 3.08 mmol, 5.0 eq), and the reaction mixture was stirred at 25° C. for 1 hour. The mixture was concentrated, and the resulting crude product was purified by flash chromatography on SiO₂ (gradient: 0˜10% methanol in dichloromethane) to afford tert-butyl 3-[8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]-7-[3-hydroxy-8-(2-triisopropylsilylethynyl)-1-naphthyl]pyrido[4,3-d]pyrimidin-4-yl]-3,8-diazabicyclo[3.2.1]octane-8-carboxylate (400 mg, 0.303 mmol, 49% yield) as a yellow solid. LC/MS (ESI) m/z: 1319.3 [M+H].

Step 4: Preparation of (2S,4R)-1-[(2R)-2-[3-[[1-[2-[4-(3,8-diazabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

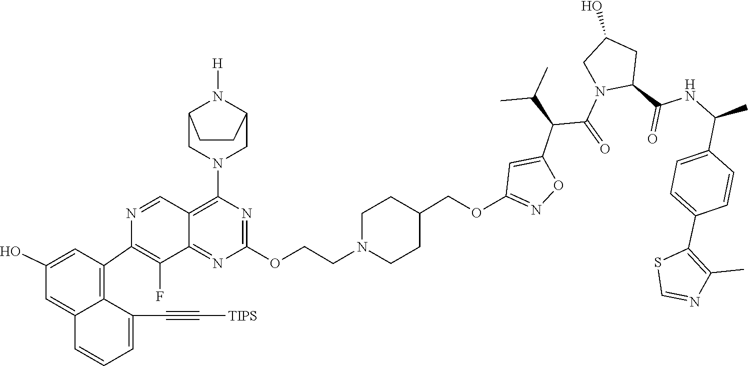

A solution of tert-butyl 3-[8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]-7-[3-hydroxy-8-(2-triisopropylsilylethynyl)-1-naphthyl]pyrido[4,3-d]pyrimidin-4-yl]-3,8-diazabicyclo[3.2.1]octane-8-carboxylate (280 mg, 0.212 mmol, 1.0 eq) in HCl/dioxane (2.5 mL) and CH₃CN (2.5 mL) was stirred at 25° C. for 1 hour. The reaction mixture was concentrated, then diluted with water (15 mL) and the pH adjusted to pH ˜8 with sat. aq. NaHCO₃. The resulting suspension was extracted with CH₂Cl₂ (20 mL×3), and the combined organic extract was washed with brine (15 mL×3), dried over Na₂SO₄, filtered, and concentrated under reduced pressure. The resulting residue was purified by prep-HPLC (column: Phenomenex C18 75*30 mm*3 um; mobile phase: [12-52% CH₃CN in water (formic acid)]). The pure fractions were lyophilized to afford (2S,4R)-1-[(2R)-2-[3-[[1-[2-[4-(3,8-diazabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (127 mg, 0.104 mmol, 49% yield) as a yellow solid. LC/MS (ESI) m/z: 1220.5 [M+H]⁺.

Step 5: Preparation (2S,4R)-1-[(2R)-2-{3-[(1-{2-[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

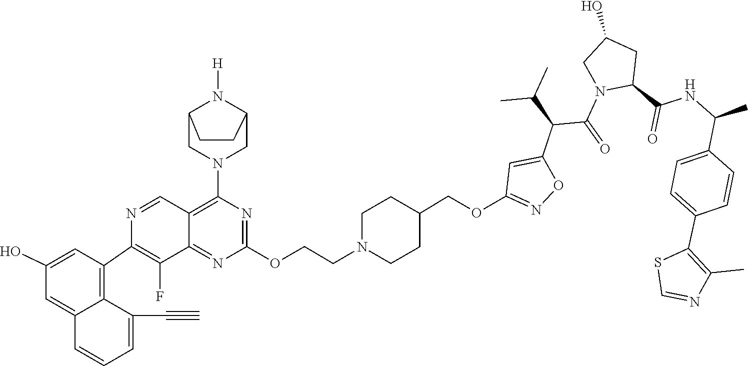 [SMILES: C#Cc1cccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of (2S,4R)-1-[(2R)-2-[3-[[1-[2-[4-(3,8-diazabicyclo[3.2.1]octan-3-yl)-8-fluoro-7-[3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (106 mg, 0.087 mmol, 1.0 eq) in DMF (2 mL) was added CsF (132 mg, 0.87 mmol, 10.0 eq), and the reaction mixture was stirred at 25° C. for 10 hours. The mixture was filtered and concentrated to afford the crude product, which was purified by prep-HPLC (column: ChiralPak IH, 250*30 mm, 10 um; mobile phase: [25% CH₃CN in EtOH (0.1% NH₄OH)]). The pure fraction was concentrated and lyophilized to afford (2S,4R)-1-[(2R)-2-{3-[(1-{2-[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (68 mg, 0.061 mmol, 71% yield) as a yellow solid. LC/MS (ESI) m/z: 1063.4 [M+H]⁺. ¹H NMR (400 MHz, CD₃OD) δ 8.99 (s, 1H), 8.92-8.84 (m, 1H), 7.80 (d, J=8.0 Hz, 1H), 7.49-7.27 (m, 7H), 7.15 (d, J=2.4 Hz, 1H), 6.04-5.86 (m, 1H), 5.05-4.98 (m, 1H), 4.66-4.38 (m, 6H), 4.04 (d, J=6.0 Hz, 2H), 3.74-3.59 (m, 6H), 3.13-3.11 (m, 2H), 3.03 (s, 1H), 2.90-2.82 (m, 2H), 2.50-2.45 (m, 3H), 2.38-2.31 (m, 1H), 2.24-2.13 (m, 3H), 1.91-1.78 (m, 7H), 1.54-1.47 (m, 3H), 1.29 (d, J=4.0 Hz, 4H), 1.05 (d, J=6.4 Hz, 3H), 0.91-0.86 (m, 3H).

Exemplary Synthesis of (2S,4R)-1-[(2R)-2-{3-[(1-{2-[(4-{3,8-diazabicyclo[3.2.1]octan-3-yl}-8-fluoro-7-[5-(trifluoromethoxy)-1H-indazol-4-yl]pyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 47)

Step 1: Preparation of 4-bromo-1H-indazol-5-ol

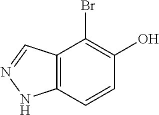 [SMILES: Oc1ccc2[nH]ncc2c1Br]

To a solution of 1H-indazol-5-ol (10.0 g, 74.55 mmol, 1.0 eq) in THF (200 mL) was added NBS (13.3 g, 74.55 mmol, 1.0 eq), and the mixture was stirred at 20° C. for 12 hours. The reaction was quenched by addition of H₂O (50 mL), and the aqueous phase was extracted with ethyl acetate (70 mL×3). The combined organic extract was washed with brine (50 mL), dried with anhydrous Na₂SO₄, filtered, and concentrated in vacuum. The resulting residue was purified by flash chromatography on SiO₂ (gradient: 0˜20% tetrahydrofuran in petroleum ether) to afford 4-bromo-1H-indazol-5-ol (16.9 g, 66.18 mmol, 89% yield) as a yellow solid. LC/MS (ESI) m/z: 213.1 [M+H]⁺.

Step 2: Preparation of 4-bromo-1-tetrahydropyran-2-yl-indazol-5-ol

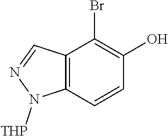

To a solution of 4-bromo-1H-indazol-5-ol (5.0 g, 23.47 mmol, 1.0 eq) and 3,4-dihydro-2H-pyran (2.58 mL, 28.16 mmol, d=920 mg/mL, 1.2 eq) in THF (50 mL) and CH₂Cl₂ (50 mL) was added methanesulfonic acid (226 mg, 2.35 mmol, 0.1 eq), and the mixture was stirred at 20° C. for 12 hours. The reaction mixture was quenched by addition of H₂O (20 mL), and the aqueous phase was extracted with dichloromethane (30 mL×3). The combined organic extracts was washed with brine (20 mL), dried with anhydrous Na₂SO₄, filtered, and concentrated in vacuum. The resulting residue was purified by flash chromatography on SiO₂ (gradient: 0˜5% ethyl acetate in petroleum ether) to afford 4-bromo-1-tetrahydropyran-2-yl-indazol-5-ol (3.9 g, 11.78 mmol, 50% yield) as a yellow solid. LC/MS (ESI) m/z: 298.7 [M+H]⁺.

Step 3: Preparation of 4-bromo-5-[bromo (difluoro)methoxy]-1-tetrahydropyran-2-yl-indazole

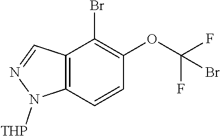

To a solution of 4-bromo-1-tetrahydropyran-2-yl-indazol-5-ol (4.8 g, 16.36 mmol, 1.0 eq) in THF at 0° C. (50 mL) was added NaH (1.5 g, 35.98 mmol, 60% purity, 2.2 eq) followed by dibromo (difluoro) methane (9.1 mL, 98.13 mmol, d=2.27 g/mL, 6.0 eq), and the reaction mixture was stirred at 20° C. for 40 hours. The reaction was quenched by addition of H₂O (50 mL), and the aqueous phase was extracted with dichloromethane (100 mL×3). The combined organic extract was washed with brine (30 mL), dried with anhydrous Na₂SO₄, filtered, and concentrated in vacuum. The resulting residue was purified by flash chromatography on SiO₂ (gradient: 0˜3% ethyl acetate in petroleum ether) to afford 4-bromo-5-[bromo (difluoro)methoxy]-1-tetrahydropyran-2-yl-indazole (2.2 g, 4.13 mmol, 25% yield) as a light yellow oil. LC/MS (ESI) m/z: 426.9 [M+H]⁺.

Step 4: Preparation of 4-bromo-5-(trifluoromethoxy)-1H-indazole

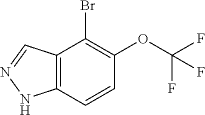 [SMILES: FC(F)(F)Oc1ccc2[nH]ncc2c1Br]

To a solution of 4-bromo-5-[bromo (difluoro)methoxy]-1-tetrahydropyran-2-yl-indazole (2.0 g, 4.02 mmol, 1.0 eq) in DCE (20 mL) was added silver tetrafluoroborate (5.5 g, 28.17 mmol, 6.0 eq), and the reaction mixture was stirred at 65° C. for 12 hours. The reaction mixture was diluted with water (20 mL) and ethyl acetate (50 mL). The resulting mixture was filtered, and the organic layer was separated. The aqueous layer was further extracted with ethyl acetate (40 mL*3), and the combined organic extracts was washed with brine (30 mL), dried over anhydrous Na₂SO₄, filtered, and concentrated over vacuum to afford 4-bromo-5-(trifluoromethoxy)-1H-indazole (1.31 g, crude) as a yellow oil. LC/MS (ESI) m/z: 281.0 [M+H]⁺.

Step 5: Preparation of tert-butyl 4-bromo-5-(trifluoromethoxy)indazole-1-carboxylate

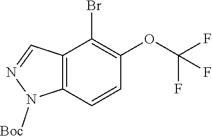 [SMILES: CC(C)(C)OC(=O)n1ncc2c(Br)c(OC(F)(F)F)ccc21]

To a solution of 4-bromo-5-(trifluoromethoxy)-1H-indazole (1.3 g, 3.64 mmol, 78% purity, 1.0 eq) and Et₃N (2.53 mL, 18.18 mmol, d=0.727 g/mL, 5.0 eq) in THF (20 mL) were added DMAP (89 mg, 0.727 mmol, 0.2 eq) and Boc₂O (1.67 mL, 7.27 mmol, d=0.95 g/mL, 2.0 eq), and the reaction mixture was stirred at 20° C. for 12 hours. The reaction was quenched with water (20 mL), then extracted with ethyl acetate (30 mL×3). The combined organic extract was washed with brine (20 mL), dried over anhydrous Na₂SO₄, filtered, and concentrated over vacuum. The resulting residue was purified by flash chromatography on SiO₂ (gradient: 0˜5% ethyl acetate in petroleum ether) to afford tert-butyl 4-bromo-5-(trifluoromethoxy)indazole-1-carboxylate (890 mg, 2.10 mmol, 52% yield) as a light yellow solid. LC/MS (ESI) m/z: 327.0 [M−55]⁺. ¹H NMR (400 MHz, CDCl₃) δ 8.23 (s, 1H), 8.19 (d, J=9.2 Hz, 1H), 7.49 (dd, J=1.2, 9.2 Hz, 1H), 1.75-1.72 (m, 9H).

Step 6: Preparation of tert-butyl 4-(4,4,5,5-tetramethyl-1,3,2-dioxaborolan-2-yl)-5-(trifluoromethoxy)indazole-1-carboxylate

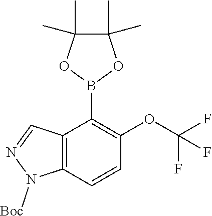 [SMILES: CC(C)(C)OC(=O)n1ncc2c(B3OC(C)(C)C(C)(C)O3)c(OC(F)(F)F)ccc21]

To a solution of tert-butyl 4-bromo-5-(trifluoromethoxy)indazole-1-carboxylate (800 mg, 2.1 mmol, 1.0 eq) and 4,4,5,5-tetramethyl-2-(4,4,5,5-tetramethyl-1,3,2-dioxaborolan-2-yl)-1,3,2-dioxaborolane (1.6 g, 6.30 mmol, 3.0 eq) in dioxane (15 mL) were added KOAc (618 mg, 6.30 mmol, 3.0 eq) and Pd(dppf) Cl₂ (154 mg, 0.210 mmol, 0.1 eq), and the mixture was stirred at 100° C. for 13 hours. The reaction mixture was diluted with ethyl acetate (20 mL), filtered, and concentrated over vacuum. The resulting residue was purified by flash chromatography on SiO₂ (gradient: 0˜3% ethyl acetate in petroleum ether) to afford tert-butyl 4-(4,4,5,5-tetramethyl-1,3,2-dioxaborolan-2-yl)-5-(trifluoromethoxy)indazole-1-carboxylate (900 mg) as a light yellow solid. LC/MS (ESI) m/z: 429.1 [M+H]⁺. ¹H NMR (400 MHz, CDCl₃) δ 8.55 (s, 1H), 8.28 (d, J=9.2 Hz, 1H), 7.42 (dd, J=0.8, 9.1=2 Hz, 1H), 1.73 (s, 9H), 1.41 (s, 12H).

Step 7: Preparation of tert-butyl 4-[4-(8-tert-butoxycarbonyl-3,8-diazabicyclo[3.2.1]octan-3-yl)-2-(2,2-dimethoxyethoxy)-8-fluoro-pyrido[4,3-d]pyrimidin-7-yl]-5-(trifluoromethoxy)indazole-1-carboxylate

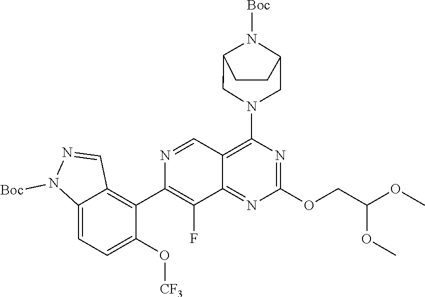 [SMILES: COC(COc1nc(N2CC3CCC(C2)N3C(=O)OC(C)(C)C)c2cnc(-c3c(OC(F)(F)F)ccc4c3cnn4C(=O)OC(C)(C)C)c(F)c2n1)OC]

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (250 mg, 0.502 mmol, 1.0 eq) and tert-butyl 4-(4,4,5,5-tetramethyl-1,3,2-dioxaborolan-2-yl)-5-(trifluoromethoxy)indazole-1-carboxylate (788 mg, 0.552 mmol, 30% purity, 1.1 eq) in dioxane (5 mL) were added K₃PO₄ (1.5 M, 0.70 mL, 2.0 eq) and XPhos Pd G3 (85 mg, 0.100 mmol, 0.2 eq), and the reaction mixture was stirred at 95° C. for 10 hours. The reaction was diluted with ethyl acetate (20 mL), filtered, and concentrated over vacuum. The resulting residue was purified by flash chromatography on SiO₂ (gradient: 0˜15% ethyl acetate in petroleum ether) to afford tert-butyl 4-[4-(8-tert-butoxycarbonyl-3,8-diazabicyclo[3.2.1]octan-3-yl)-2-(2,2-dimethoxyethoxy)-8-fluoro-pyrido[4,3-d]pyrimidin-7-yl]-5-(trifluoromethoxy)indazole-1-carboxylate (170 mg, 0.183 mmol, 36% yield) as a yellow oil. LC/MS (ESI) m/z: 764.7 [M+H]⁺.

Step 8: Preparation of tert-butyl 3-[8-fluoro-2-(2-oxoethoxy)-7-[5-(trifluoromethoxy)-1H-indazol-4-yl]pyrido[4,3-d]pyrimidin-4-yl]-3,8-diazabicyclo[3.2.1]octane-8-carboxylate

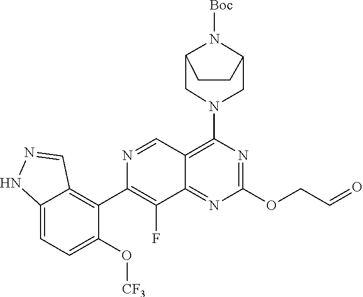 [SMILES: CC(C)(C)OC(=O)N1C2CCC1CN(c1nc(OCC=O)nc3c(F)c(-c4c(OC(F)(F)F)ccc5[nH]ncc45)ncc13)C2]

To a solution of tert-butyl 4-[4-(8-tert-butoxycarbonyl-3,8-diazabicyclo[3.2.1]octan-3-yl)-2-(2,2-dimethoxyethoxy)-8-fluoro-pyrido[4,3-d]pyrimidin-7-yl]-5-(trifluoromethoxy)indazole-1-carboxylate (155 mg, 0.165 mmol, 1.0 eq) in acetone (0.34 mL) was added aqueous HCl (12 M, 0.34 mL, 20.0 eq), and the reaction mixture was stirred at 20° C. for 10 minutes. A solution of NaHCO₃ (682 mg, 8.12 mmol, 40.0 eq) in H₂O (2 mL) and THF (2 mL) was added followed by Boc₂O (0.093 mL, 0.406 mmol, d=0.95 g/mL, 2.0 eq), and the reaction mixture was stirred at 20° C. for 1 hours. The reaction was diluted with water (10 mL) and extracted with ethyl acetate (30 mL×3). The combined organic extract was washed with brine (20 mL), dried over anhydrous Na₂SO₄, filtered, and concentrated over vacuum. The resulting residue was purified by flash chromatography on SiO₂ (gradient: 0˜75% ethyl acetate in petroleum ether) to afford tert-butyl 3-[8-fluoro-2-(2-oxoethoxy)-7-[5-(trifluoromethoxy)-1H-indazol-4-yl]pyrido[4,3-d]pyrimidin-4-yl]-3,8-diazabicyclo[3.2.1]octane-8-carboxylate (150 mg, 0.170 mmol, 84% yield) as a yellow solid. LC/MS (ESI) m/z: 618.2 [M+H].

Step 9: Preparation of (2S,4R)-1-[(2R)-2-{3-[(1-{2-[(4-{3,8-diazabicyclo[3.2.1]octan-3-yl}-8-fluoro-7-[5-(trifluoromethoxy)-1H-indazol-4-yl]pyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

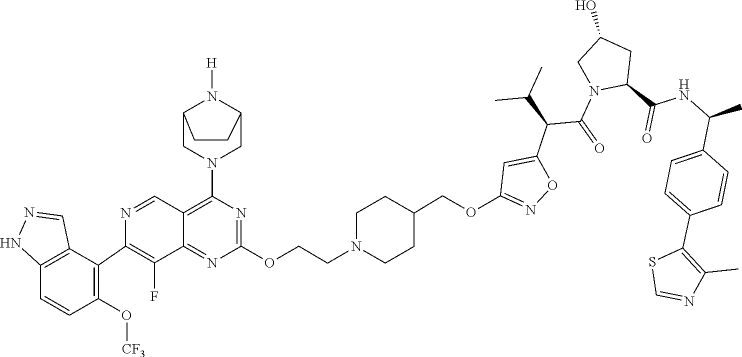 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6c(OC(F)(F)F)ccc7[nH]ncc67)c(F)c5n4)CC3)no2)C(C)C)cc1]

The title compound was prepared in an analogous manner to Compound 20 starting from tert-butyl 3-[8-fluoro-2-(2-oxoethoxy)-7-[5-(trifluoromethoxy)-1H-indazol-4-yl]pyrido[4,3-d]pyrimidin-4-yl]-3,8-diazabicyclo[3.2.1]octane-8-carboxylate and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide, and purified by pre-HPLC (Condition: 5-45% CH₃CN in water (formic acid)). The pure fractions were combined and lyophilized to afford (2S,4R)-1-[(2R)-2-{3-[(1-{2-[(4-{3,8-diazabicyclo[3.2.1]octan-3-yl}-8-fluoro-7-[5-(trifluoromethoxy)-1H-indazol-4-yl]pyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (52.4 mg, 0.048 mmol, 55% yield, FA) as a white solid. LC/MS (ESI) m/z: 1097.3 [M+H]⁺. ¹H NMR (400 MHz, CD₃OD) δ 9.21 (s, 1H), 8.88 (s, 1H), 8.52 (s, 1H), 7.98 (s, 1H), 7.81 (dd, J=0.8, 9.2 Hz, 1H), 7.53 (dd, J=1.2, 9.2 Hz, 1H), 7.48-7.34 (m, 4H), 6.06-5.89 (m, 1H), 5.08-4.97 (m, 1H), 4.79-4.71 (m, 4H), 4.51 (t, J=8.4 Hz, 1H), 4.46-4.37 (m, 1H), 4.08 (d, J=6.0 Hz, 2H), 3.91-3.72 (m, 5H), 3.69-3.45 (m, 2H), 3.40 (d, J=11.2 Hz, 2H), 3.24-3.15 (m, 2H), 2.60 (t, J=12.0 Hz, 2H), 2.53-2.43 (m, 3H), 2.41-2.30 (m, 1H), 2.22-2.14 (m, 1H), 2.04-1.87 (m, 8H), 1.60-1.48 (m, 5H), 1.05 (d, J=6.4 Hz, 3H), 0.92-0.85 (m, 3H).

Exemplary Synthesis of [(3R,7aR)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 46)

Step 1: Preparation of 4-(azepan-1-yl)-2,7-dichloro-8-fluoro-pyrido[4,3-d]pyrimidine

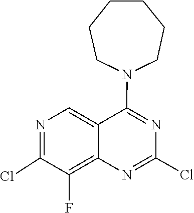 [SMILES: Fc1c(Cl)ncc2c(N3CCCCCC3)nc(Cl)nc12]

To a solution of 2,4,7-trichloro-8-fluoro-pyrido[4,3-d]pyrimidine (5.8 g, 22.97 mmol, 1.0 eq) in CH₂Cl₂ (80 mL) at −40° C. were added DIEA (137.84 mmol, 24.0 mL, 6.0 eq) and azepane (2.3 g, 22.97 mmol, 2.6 mL, 1.0 eq) dropwise, and the reaction mixture was stirred at −40° C. for 0.5 hour. The reaction was quenched by addition of water (20 mL) and stirred for 20 minutes. The layers were separated, and the aqueous layer was further extracted with dichloromethane (50 mL). The combined organic extract was washed with saturated aqueous NaHCO₃ (50 mL) followed by brine (50 mL), dried over anhydrous sodium sulfate, filtered, and concentrated. The resulting residue was purified by flash chromatography on SiO₂ (gradient: 0˜15% THF in petroleum ether) to afford 4-(azepan-1-yl)-2,7-dichloro-8-fluoro-pyrido[4,3-d]pyrimidine (5.2 g, 14.85 mmol, 65% yield) as a yellow solid. LC/MS (ESI) m/z: 315.6 [M+H]⁺.

Step 2: Preparation of [(3R,8R)-8-[[4-(azepan-1-yl)-7-chloro-8-fluoro-pyrido[4,3-d]pyrimidin-2-yl]oxymethyl]-1,2,3,5,6,7-hexahydropyrrolizin-3-yl]methoxy-tert-butyl-diphenyl-silane

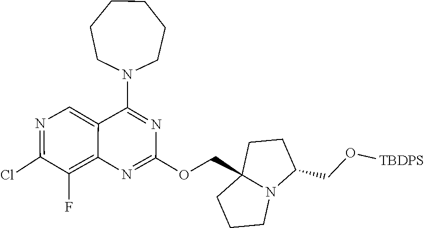

To a solution of 4-(azepan-1-yl)-2,7-dichloro-8-fluoro-pyrido[4,3-d]pyrimidine (850 mg, 2.70 mmol, 1.0 eq) and [(3R,8R)-3-[[tert-butyl(diphenyl)silyl]oxymethyl]-1,2,3,5,6,7-hexahydropyrrolizin-8-yl]methanol (1.1 g, 2.70 mmol, 1.0 eq) in THF (30 mL) at 0° C. was added NaH (173 mg, 4.32 mmol, 60% purity, 1.6 eq), and the reaction mixture was stirred at 15° C. for 15 hours. The reaction mixture was quenched by addition of saturated aqueous NH₄Cl (15 mL), then extracted with ethyl acetate (40 mL×3). The combined organic extract was washed with brine (20 mL), dried over anhydrous sodium sulfate, filtered, and concentrated. The resulting residue was purified by flash chromatography on SiO₂ (gradient: 0˜18% THF in petroleum ether) to afford [(3R,8R)-8-[[4-(azepan-1-yl)-7-chloro-8-fluoro-pyrido[4,3-d]pyrimidin-2-yl]oxymethyl]-1,2,3,5,6,7-hexahydropyrrolizin-3-yl]methoxy-tert-butyl-diphenyl-silane (1.1 g, 1.44 mmol, 53% yield) as a yellow solid. LC/MS (ESI) m/z: 688.4 [M+H]⁺.

Step 3: Preparation of 2-[8-[[4-(azepan-1-yl)-2-[[(3R,8R)-3-[[tert-butyl(diphenyl)silyl]oxymethyl]-1,2,3,5,6,7-hexahydropyrrolizin-8-yl]methoxy]-8-fluoro-pyrido[4,3-d]pyrimidin-7-yl]-6-(methoxymethoxy)-1-naphthyl]ethynyl-triisopropyl-silane

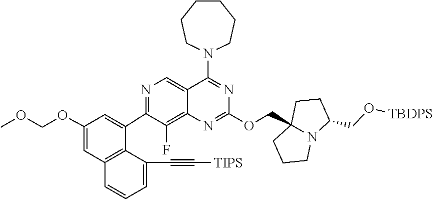

To a solution of [(3R,8R)-8-[[4-(azepan-1-yl)-7-chloro-8-fluoro-pyrido[4,3-d]pyrimidin-2-yl]oxymethyl]-1,2,3,5,6,7-hexahydropyrrolizin-3-yl]methoxy-tert-butyl-diphenyl-silane (1.0 g, 1.45 mmol, 1.0 eq) and triisopropyl-[2-[6-(methoxymethoxy)-8-(4,4,5,5-tetramethyl-1,3,2-dioxaborolan-2-yl)-1-naphthyl]ethynyl]silane (1.0 g, 2.03 mmol, 1.4 eq) in dioxane (10 mL) and H₂O (2 mL) were added K₃PO₄ (925 mg, 4.36 mmol, 3.0 eq) and CATACXIUM® A PD G3 (211 mg, 290.55 umol, 0.2 eq), and the reaction mixture was stirred at 85° C. for 7 hours under N₂. The mixture was diluted with ethyl acetate (50 mL), then washed with water (30 mL×3). The organic extract was washed with brine (30 mL), dried over anhydrous sodium sulfate, filtered and concentrated in vacuum. The resulting residue was purified by flash chromatography on SiO₂ (gradient: 0˜51% ethyl acetate in petroleum ether) to afford 2-[8-[[4-(azepan-1-yl)-2-[[(3R,8R)-3-[[tert-butyl(diphenyl)silyl]oxymethyl]-1,2,3,5,6,7-hexahydropyrrolizin-8-yl]methoxy]-8-fluoro-pyrido[4,3-d]pyrimidin-7-yl]-6-(methoxymethoxy)-1-naphthyl]ethynyl-triisopropyl-silane (1.0 g, 881.95 umol, 61% yield) as a yellow oil. LC/MS (ESI) m/z: 511.1 [M/2+H]⁺.

Step 4: Preparation of [(3R,8R)-8-[[4-(azepan-1-yl)-7-[8-ethynyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol

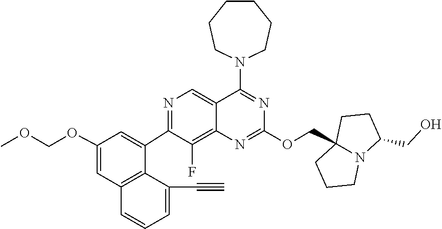 [SMILES: C#Cc1cccc2cc(OCOC)cc(-c3ncc4c(N5CCCCCC5)nc(OC[C@]56CCCN5[C@@H](CO)CC6)nc4c3F)c12]

To a solution of 2-[8-[[4-(azepan-1-yl)-2-[[(3R,8R)-3-[[tert-butyl(diphenyl)silyl]oxymethyl]-1,2,3,5,6,7-hexahydropyrrolizin-8-yl]methoxy]-8-fluoro-pyrido[4,3-d]pyrimidin-7-yl]-6-(methoxymethoxy)-1-naphthyl]ethynyl-triisopropyl-silane (700 mg, 685.96 umol, 1.0 eq) in THF (8 mL) was added TBAF (1 M, 1.5 mL, 2.2 eq), and the reaction mixture was stirred at 15° C. for 15 hours. The mixture was diluted with ethyl acetate (30 mL) and washed with water (20 mL×5). The organic extract was washed with brine 30 mL, dried over Na₂SO₄, filtered, and concentrated to afford the crude product [(3R,8R)-8-[[4-(azepan-1-yl)-7-[8-ethynyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (1.0 g, crude) as a brown oil. LC/MS (ESI) m/z: 626.3 [M+H]⁺.

Step 5: Preparation of [(3R,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol

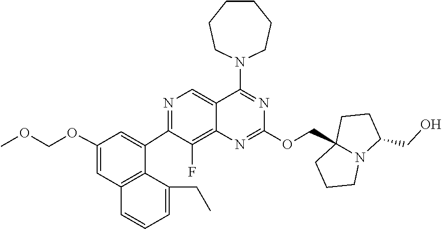 [SMILES: CCc1cccc2cc(OCOC)cc(-c3ncc4c(N5CCCCCC5)nc(OC[C@]56CCCN5[C@@H](CO)CC6)nc4c3F)c12]

To a solution of [(3R,8R)-8-[[4-(azepan-1-yl)-7-[8-ethynyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (800 mg, 1.28 mmol, 1.0 eq) in CH₃OH (20 mL) was added Pd/C (300 mg, 10% purity), and the reaction mixture was stirred at 15° C. for 1 hour under H₂ (15 psi). The mixture was filtered, then concentrated. The resulting residue was purified by flash chromatography on SiO₂ (gradient: 0˜13% (NH₃—CH₃OH/CH₃OH= 1/10) in CH₂Cl₂) to afford [(3R,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (350 mg, 511.30 umol, 40% yield) as a brown oil. LC/MS (ESI) m/z: 630.3 [M+H]⁺.

Step 6: Preparation of [(3R,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

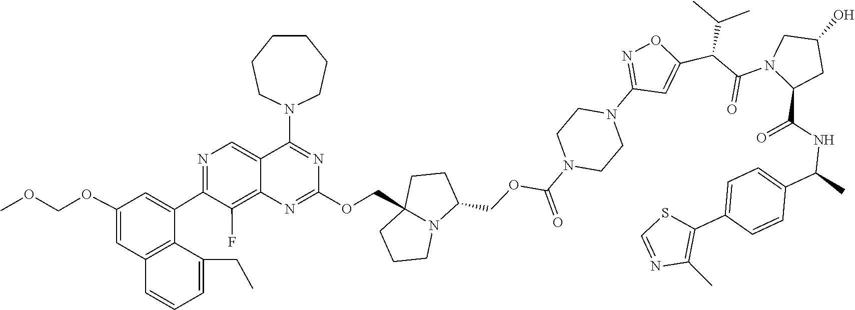 [SMILES: CCc1cccc2cc(OCOC)cc(-c3ncc4c(N5CCCCCC5)nc(OC[C@]56CCCN5[C@@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of [(3R,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (175 mg, 277.88 umol, 1.0 eq) and (4-nitrophenyl) carbonochloridate (112 mg, 555.77 umol, 2.0 eq) in THF (5 mL) were added triethylamine (2.22 mmol, 309 μL, 8.0 eq) and DMAP (3.4 mg, 27.79 umol, 0.1 eq), and the reaction mixture was stirred at 15° C. for 15 hours under N₂. (2S,4R)-4-Hydroxy-1-[(2S)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (173 mg, 305.67 umol, 1.1 eq) was then added, and the reaction mixture was stirred at 15° C. for 1 hour. The mixture was filtered, then concentrated, and the resulting residue was purified by flash chromatography on SiO₂ (gradient: 0˜4% MeOH in CH₂Cl₂) to afford [(3R,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (300 mg, 220.86 umol, 79% yield) as a brown solid. LC/MS (ESI) m/z: 612.1 [M/2+H]⁺.

Step 7: Preparation of [(3R,7aR)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

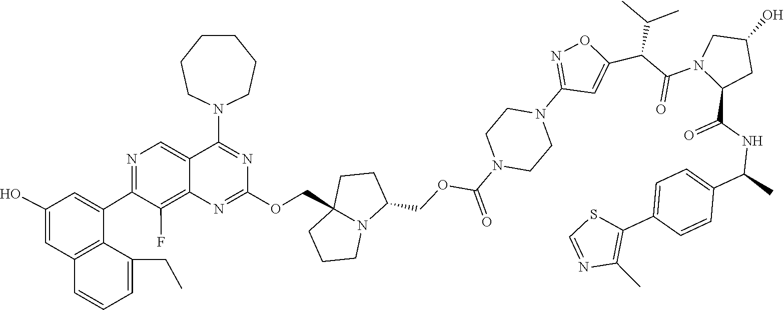 [SMILES: CCc1cccc2cc(O)cc(-c3ncc4c(N5CCCCCC5)nc(OC[C@]56CCCN5[C@@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

A solution of [(3R,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (300 mg, 245.40 umol, 1.0 eq) in formic acid (3 mL) was stirred at 15° C. for 3 hours. The mixture was concentrated, and the resulting residue was purified by prep. HPLC (gradient: 15˜55% acetonitrile in water (0.225% FA)). The pure fractions were combined and lyophilized under reduced pressure to afford product [(3R,7aR)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (48.0 mg, 40.16 umol, 16% yield, FA) as a light yellow solid. LC/MS (ESI) m/z: 1178.9. [M+H]⁺. ¹H NMR (400 MHz, CD₃OD) δ 9.16-9.15 (m, 1H), 8.88-8.86 (m, 1H), 7.61 (d, J=8.0 Hz, 1H), 7.37-7.32 (m, 5H), 7.28 (d, J=2.4 Hz, 1H), 7.16-7.14 (m, 1H), 7.00 (dd, J=2.4 Hz, 1H), 6.14 (s, 1H), 4.98 (t, J=7.6 Hz, 1H), 4.71-4.66 (m, 2H), 4.59-4.50 (m, 4H), 4.42-4.34 (m, 2H), 4.21 (s, 1H), 4.12-4.09 (m, 4H), 3.76-3.64 (m, 3H), 3.54-3.48 (m, 5H), 3.15 (s, 3H), 2.44 (d, J=3.2 Hz, 3H), 2.41-2.18 (m, 8H), 2.10-2.04 (m, 8H), 1.98-1.91 (m, 1H), 1.70 (s, 4H), 1.46 (dd, J=9.2 Hz, 3H), 1.05 (d, J=8.0 Hz, 3H), 0.91-0.86 (m, 6H).

Exemplary Synthesis of [(3R,7aR)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 45)

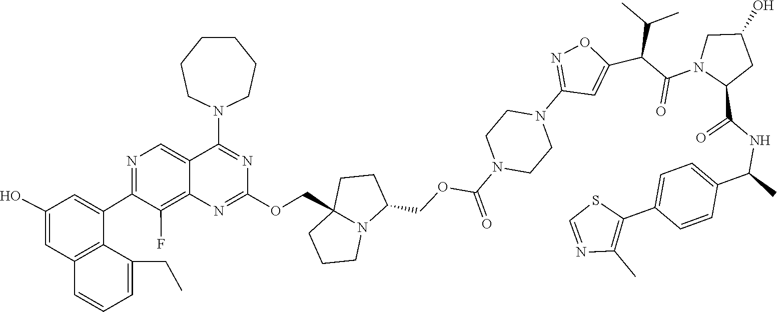 [SMILES: CCc1cccc2cc(O)cc(-c3ncc4c(N5CCCCCC5)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 46 starting from [(3R,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-4-hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide, and purified by prep. HPLC (gradient: 15˜55% acetonitrile in water (0.225% FA)) The pure fractions were combined and lyophilized under reduced pressure to afford product [(3R,7aR)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (59.8 mg, 49.83 umol, 21% yield, FA) as a white solid. LC/MS (ESI) m/z: 1178.9. [M+H]⁺. ¹H NMR (400 MHz, CD₃OD) δ 9.16-9.15 (m, 1H), 8.88-8.86 (m, 1H), 7.62 (d, J=8.0 Hz, 1H), 7.45-7.34 (m, 5H), 7.29-7.28 (m, 1H), 7.17-7.14 (m, 1H), 7.02 (d, J=2.4 Hz, 1H), 6.13-6.12 (m, 1H), 5.06-4.99 (m, 1H), 4.66-4.60 (m, 2H), 4.53-4.46 (m, 3H), 4.43-4.35 (m, 2H), 4.14-4.11 (m, 5H), 3.85-3.76 (m, 1H), 3.65-3.40 (m, 7H), 3.22 (s, 4H), 2.48-2.46 (m, 3H), 2.40-2.14 (m, 8H), 2.05-1.92 (m, 9H), 1.72 (s, 4H), 1.58-1.50 (m, 3H), 1.05 (d, J=8.0 Hz, 3H), 0.92-0.86 (m, 6H).

Exemplary Synthesis of [(3S,7aS)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 42)

Step 1: Preparation of [(3S,8S)-8-[[4-(azepan-1-yl)-7-chloro-8-fluoro-pyrido[4,3-d]pyrimidin-2-yl]oxymethyl]-1,2,3,5,6,7-hexahydropyrrolizin-3-yl]methoxy-tert-butyl-diphenyl-silane

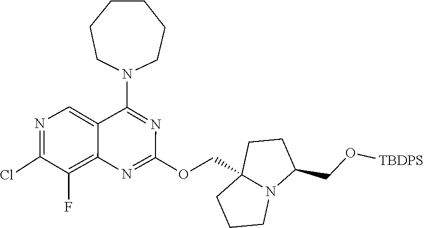

To a solution of 4-(azepan-1-yl)-2,7-dichloro-8-fluoro-pyrido[4,3-d]pyrimidine (725 mg, 2.30 mmol, 1.0 eq) and [(3S,8S)-3-[[tert-butyl(diphenyl)silyl]oxymethyl]-1,2,3,5,6,7-hexahydropyrrolizin-8-yl]methanol (992 mg, 2.30 mmol, 1.0 eq) in tetrahydrofuran (20 mL) at 0° C. was added sodium hydride (147 mg, 3.68 mmol, 60% purity, 1.6 eq), and the reaction mixture was stirred at 25° C. for 16 hours. The reaction was quenched by addition of saturated aqueous NH₄Cl (15 mL), then extracted with ethyl acetate (40 mL×3). The combined organic extracts were washed with brine (20 mL), dried over anhydrous Na₂SO₄, and concentrated under reduced pressure. The crude product was purified by flash chromatography on SiO₂ (gradient: 0˜3% methanol in dichloromethane) to afford [(3S,8S)-8-[[4-(azepan-1-yl)-7-chloro-8-fluoro-pyrido[4,3-d]pyrimidin-2-yl]oxymethyl]-1,2,3,5,6,7-hexahydropyrrolizin-3-yl]methoxy-tert-butyl-diphenyl-silane (734 mg, 1.01 mmol, 44% yield) as a light yellow solid. LC/MS (ESI) m/z: 688.4 [M+H]⁺. ¹H NMR (400 MHz, CDCl₃) δ 8.85 (s, 1H), 7.65-7.62 (m, 4H), 7.47-7.40 (m, 6H), 4.24 (d, J=5.6 Hz, 1H), 3.99-3.92 (m, 5H), 3.84-3.49 (m, 2H), 2.55-2.37 (m, 1H), 2.27-2.10 (m, 4H), 2.01 (s, 5H), 1.66 (s, 4H), 1.61 (s, 5H), 1.10-1.03 (m, 9H).

Step 2: Preparation of 2-[8-[[4-(azepan-1-yl)-2-[[(3S,8S)-3-[[tert-butyl(diphenyl)silyl]oxymethyl]-1,2,3,5,6,7-hexahydropyrrolizin-8-yl]methoxy]-8-fluoro-pyrido[4,3-d]pyrimidin-7-yl]-6-(methoxymethoxy)-1-naphthyl]ethynyl-triisopropyl-silane

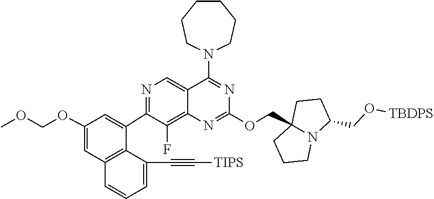

To a solution of [(3S,8S)-8-[[4-(azepan-1-yl)-7-chloro-8-fluoro-pyrido[4,3-d]pyrimidin-2-yl]oxymethyl]-1,2,3,5,6,7-hexahydropyrrolizin-3-yl]methoxy-tert-butyl-diphenyl-silane (734 mg, 1.07 mmol, 1.0 eq) and triisopropyl-[2-[6-(methoxymethoxy)-8-(4,4,5,5-tetramethyl-1,3,2-dioxaborolan-2-yl)-1-naphthyl]ethynyl]silane (738 mg, 1.49 mmol, 1.4 eq) in dioxane (10 mL) and water (2 mL) were added potassium phosphate (679 mg, 3.20 mmol, 3.0 eq) and CATACXIUM® A PD G3 (155 mg, 0.21 mmol, 0.2 eq), and the reaction mixture was stirred at 85° C. for 7 hours. The reaction mixture was diluted with ethyl acetate (50 mL), and then washed with water (30 mL×3). The combined organic extracts were washed with brine (30 mL), dried over anhydrous Na₂SO₄, and concentrated under reduced pressure. The resulting crude product was purified by flash chromatography on SiO₂ (gradient: 0˜40% ethyl acetate in petroleum ether) to afford 2-[8-[[4-(azepan-1-yl)-2-[[(3S,8S)-3-[[tert-butyl(diphenyl)silyl]oxymethyl]-1,2,3,5,6,7-hexahydropyrrolizin-8-yl]methoxy]-8-fluoro-pyrido[4,3-d]pyrimidin-7-yl]-6-(methoxymethoxy)-1-naphthyl]ethynyl-triisopropyl-silane (708 mg, 0.65 mmol, 60% yield) as a yellow oil. LC/MS (ESI) m/z: 1020.6 [M+H]⁺. ¹H NMR (400 MHz, CDCl₃) δ 7.82-7.33 (m, 16H), 5.38-5.29 (m, 2H), 4.26-3.92 (m, 7H), 3.58 (d, J=1.6 Hz, 1H), 3.52 (s, 3H), 2.06 (s, 2H), 1.58 (s, 16H), 1.29-1.25 (m, 4H), 1.16-1.04 (m, 13H), 0.90-0.85 (m, 14H).

Step 3: Preparation of [(3S,8S)-8-[[4-(azepan-1-yl)-7-[8-ethynyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol

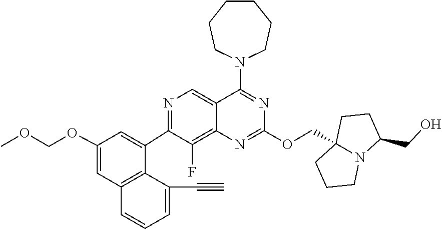 [SMILES: C#Cc1cccc2cc(OCOC)cc(-c3ncc4c(N5CCCCCC5)nc(OC[C@@]56CCCN5[C@H](CO)CC6)nc4c3F)c12]

To a solution of 2-[8-[[4-(azepan-1-yl)-2-[[(3S,8S)-3-[[tert-butyl(diphenyl)silyl]oxymethyl]-1,2,3,5,6,7-hexahydropyrrolizin-8-yl]methoxy]-8-fluoro-pyrido[4,3-d]pyrimidin-7-yl]-6-(methoxymethoxy)-1-naphthyl]ethynyl-triisopropyl-silane (708 mg, 0.69 mmol, 1.0 eq) in tetrahydrofuran (8 mL) was added tetrabutylammonium fluoride (1 M, 1.53 mL, 2.2 eq), and the reaction mixture was stirred at 20° C. for 15 hours. The reaction mixture was diluted with ethyl acetate (40 mL), and then washed with water (10 mL×5). The combined organic extracts were washed with brine (30 mL), dried over anhydrous Na₂SO₄, and concentrated under reduced pressure to afford [(3S,8S)-8-[[4-(azepan-1-yl)-7-[8-ethynyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (680 mg, crude) as a yellow oil. LC/MS (ESI) m/z: 626.3 [M+H]⁺.

Step 4: Preparation of [(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol

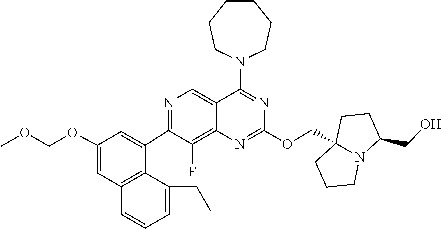 [SMILES: CCc1cccc2cc(OCOC)cc(-c3ncc4c(N5CCCCCC5)nc(OC[C@@]56CCCN5[C@H](CO)CC6)nc4c3F)c12]

To a solution of [(3S,8S)-8-[[4-(azepan-1-yl)-7-[8-ethynyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (680 mg, 1.09 mmol, 1.0 eq) in methanol (8 mL) was added Pd/C (231 mg, 0.22 mmol, 10% purity, 0.2 eq) under N₂ atmosphere, and the resulting suspension was degassed and purged with H₂ (3×). The reaction mixture was stirred under H₂ (15 psi) at 20° C. for 1 hour. The mixture was filtered, and the filtrate was concentrated under reduced pressure. The resulting crude product was purified by flash chromatography on SiO₂ (gradient: 0˜9% methanol in dichloromethane) to afford [(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (164 mg, 0.25 mmol, 23% yield) as a brown solid. LC/MS (ESI) m/z: 630.4 [M+H]⁺. ¹H NMR (400 MHz, CDCl₃) δ 7.70 (d, J=4.0 Hz, 1H), 7.54 (d, J=1.2 Hz, 1H), 7.44-7.40 (m, 1H), 7.36-7.30 (m, 1H), 7.21 (d, J=1.2 Hz, 1H), 5.32-5.30 (m, 2H), 4.12-4.03 (m, 7H), 3.56-3.55 (m, 1H), 3.53-2.52 (m, 3H), 3.18-3.11 (m, 1H), 2.54-2.34 (m, 5H), 1.73 (s, 16H), 1.01-0.97 (m, 4H).

Step 5: Preparation of [(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

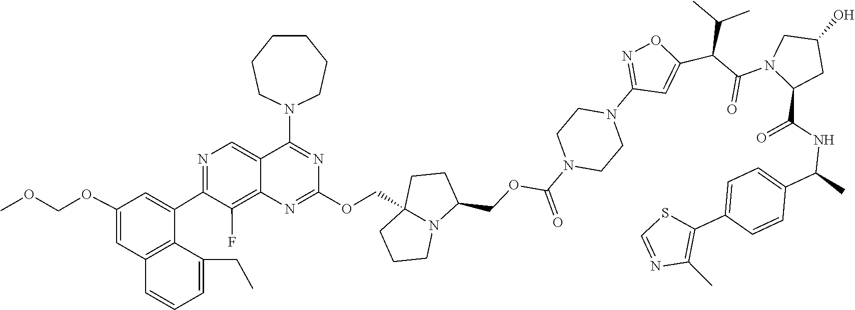 [SMILES: CCc1cccc2cc(OCOC)cc(-c3ncc4c(N5CCCCCC5)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of [(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (80 mg, 0.13 mmol, 1.0 eq) and (4-nitrophenyl) carbonochloridate (51 mg, 0.25 mmol, 2.0 eq) in tetrahydrofuran (2.5 mL) were added triethylamine (103 mg, 1.02 mmol, 0.14 mL, 8.0 eq) and DMAP (2 mg, 0.01 mmol, 0.1 eq), and the reaction mixture was stirred at 15° C. for 15 hours under N₂. (2S,4R)-4-Hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (79 mg, 0.14 mmol, 1.1 eq) was then added, and the resulting suspension was stirred at 15° C. for 1 hour. The mixture was filtered, and the filtrate was concentrated under reduced pressure. The resulting crude product was purified by flash chromatography on SiO₂ (gradient: 0˜9% methanol in dichloromethane) to afford [(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (105 mg, 0.08 mmol, 63% yield) as a brown solid. LC/MS (ESI) m/z: 1223.7 [M+H]⁺. ¹H NMR (400 MHz, CDCl₃) δ 8.93 (s, 1H), 8.15 (s, 1H), 7.69 (d, J=3.2 Hz, 2H), 7.57 (s, 1H), 7.44-7.38 (m, 7H), 6.72 (d, J=3.6 Hz, 1H), 5.92 (s, 1H), 5.31-5.30 (m, 3H), 5.07-5.04 (m, 1H), 4.71-4.45 (m, 6H), 3.81-3.64 (m, 5H), 3.54-3.46 (m, 9H), 3.25 (s, 3H), 3.14-3.08 (m, 4H), 2.61 (s, 5H), 2.47-2.35 (m, 10H), 1.48 (d, J=3.2 Hz, 4H), 1.43 (t, J=7.6 Hz, 7H), 1.03-0.99 (m, 3H), 0.94-0.87 (m, 6H).

Step 6: Preparation of [(3S,7aS)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

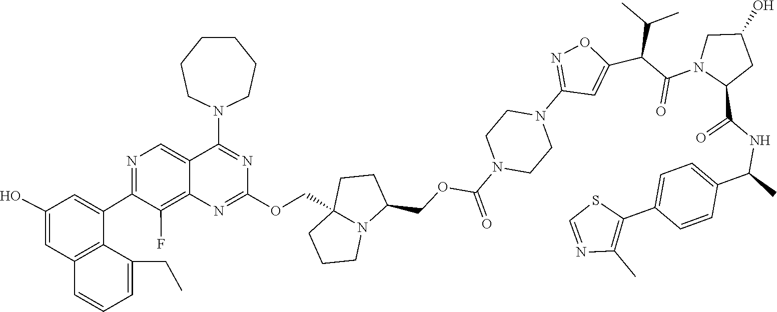 [SMILES: CCc1cccc2cc(O)cc(-c3ncc4c(N5CCCCCC5)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

A solution of [(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (105 mg, 0.09 mmol, 1.0 eq) in formic acid (8 mL) was stirred at 25° C. for 1 hour. The mixture was concentrated in vacuo at 25° C., and the resulting residue was purified by prep-HPLC (column: Phenomenex C18 75*30 mm*3 um; mobile phase: [22-62% CH₃CN in water (FA)]; Gradient Time: 28 min, Flow Rate: 25 mL/min). The pure fractions were combined and dried by lyophilization to afford [(3S,7aS)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (44.4 mg, 0.04 mmol, 42% yield, FA) as a white solid. LC/MS (ESI) m/z: 1178.9 [M+H]⁺. ¹H NMR (400 MHz, CD₃OD) δ 9.17 (s, 1H), 8.88 (s, 1H), 7.62 (d, J=4.0 Hz, 1H), 7.45-7.34 (m, 5H), 7.29-7.28 (m, 1H), 7.17-7.16 (m, 1H), 7.03-7.02 (m, 1H), 6.13 (s, 1H), 5.06-4.99 (m, 1H), 4.68-4.65 (m, 1H), 4.56-4.48 (m, 3H), 4.44-4.38 (m, 2H), 4.14-4.11 (m, 5H), 3.86-3.82 (m, 1H), 3.66-3.47 (m, 7H), 3.21 (s, 4H), 2.48 (s, 2H), 2.46 (s, 1H), 2.40-1.92 (m, 18H), 1.72 (s, 4H), 1.57 (d, J=3.2 Hz, 1H), 1.51 (d, J=3.2 Hz, 2H), 1.05 (d, J=4.0 Hz, 3H), 0.91-0.87 (m, 6H).

Exemplary Synthesis of [(3S,7aS)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 44)

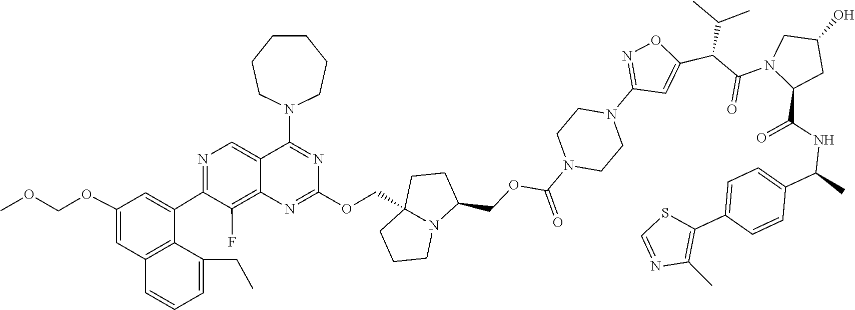 [SMILES: CCc1cccc2cc(OCOC)cc(-c3ncc4c(N5CCCCCC5)nc(OC[C@@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 42 starting from [(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and 2S,4R)-4-Hydroxy-1-[(2S)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide, and purified by prep-HPLC (column: Phenomenex C18 75*30 mm*3 um; mobile phase: [20-60% CH₃CN in water (FA)]; Gradient Time: 28 min, Flow Rate: 25 mL/min). The pure fractions were combined and dried by lyophilization to afford [(3S,7aS)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methyl 4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (25.6 mg, 0.02 mmol, 42% yield, FA) as a white solid. LC/MS (ESI) m/z: 1178.5 [M+H]⁺. ¹H NMR (400 MHz, CD₃OD) δ 9.17 (s, 1H), 8.88 (s, 1H), 7.62 (d, J=4.0 Hz, 1H), 7.38-7.32 (m, 5H), 7.28-7.27 (m, 1H), 7.16-7.15 (m, 1H), 7.01-7.00 (m, 1H), 6.14 (s, 1H), 4.98-4.94 (m, 1H), 4.70-4.65 (m, 1H), 4.57-4.47 (m, 3H), 4.42 (s, 2H), 4.13-4.11 (m, 5H), 3.76-3.63 (m, 4H), 3.54-3.47 (m, 4H), 3.21-3.13 (m, 4H), 2.48-2.45 (m, 3H), 2.39-1.94 (m, 18H), 1.72 (s, 4H), 1.59-1.57 (m, 1H), 1.47 (d, J=3.6 Hz, 2H), 1.05 (d, J=3.2 Hz, 3H), 0.91-0.87 (m, 6H).

Exemplary Synthesis of [(3R,7aR)-7a-{[4-(azepan-1-yl)-7-(8-ethylnaphthalen-1-yl)-8-fluoropyrido[4,3-d]pyrimidin-2-yl]oxy}-hexahydro-1H-pyrrolizin-3-yl]methyl 4-{5-[(2R*)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 49)

Step 1: Preparation of 4-(azepan-1-yl)-7-(8-ethyl-1-naphthyl)-8-fluoro-2-methylsulfanyl-pyrido[4,3-d]pyrimidine

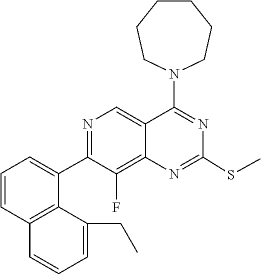 [SMILES: CCc1cccc2cccc(-c3ncc4c(N5CCCCCC5)nc(SC)nc4c3F)c12]

To a solution of 4-(azepan-1-yl)-7-chloro-8-fluoro-2-methylsulfanyl-pyrido[4,3-d]pyrimidine (520 mg, 1.59 mmol, 1 eq) and 2-(8-ethyl-1-naphthyl)-4,4,5,5-tetramethyl-1,3,2-dioxaborolane (539 mg, 1.91 mmol, 1.2 eq) in dioxane (10 mL) were added potassium phosphate (3 M, 2 mL, 3 eq) and methanesulfonato (diadamantyl-n-butylphosphino)-2′-amino-1,1′-biphenyl-2-yl) palladium (II) (116 mg, 0.16 mmol, 0.1 eq), and the reaction mixture was stirred at 80° C. for 16 h. The mixture was diluted with ethyl acetate (20 mL) at 25° C. and extracted with water (15 mL×2). The organic extract was washed with brine (20 mL×2), dried over anhydrous sodium sulfate, filtered, and concentrated. The resulting residue was purified by flash silica gel chromatography (gradient: 0˜25% EtOAc in petroleum ether) to afford 4-(azepan-1-yl)-7-(8-ethyl-1-naphthyl)-8-fluoro-2-methylsulfanyl-pyrido[4,3-d]pyrimidine (413 mg, 0.92 mmol, 58% yield) as a white solid. LC/MS (ESI) m/z: 447.2 [M+H]⁺.

Step 2: Preparation of 4-(azepan-1-yl)-7-(8-ethyl-1-naphthyl)-8-fluoro-2-methylsulfonyl-pyrido[4,3-d]pyrimidine

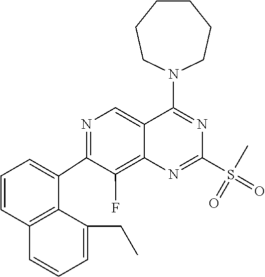 [SMILES: CCc1cccc2cccc(-c3ncc4c(N5CCCCCC5)nc(S(C)(=O)=O)nc4c3F)c12]

To a solution of 4-(azepan-1-yl)-7-(8-ethyl-1-naphthyl)-8-fluoro-2-methylsulfanyl-pyrido[4,3-d]pyrimidine (500 mg, 1.12 mmol, 1 eq) in DMF (10 mL) was added 4 A MS (750 mg), and the mixture was stirred at 25° C. for 5 h. Oxone (2.75 g, 4.48 mmol, 4 eq) was then added, and the reaction mixture was stirred at 25° C. for 11 h. The mixture was diluted with water (200 mL) and extracted with ethyl acetate (100 mL×2). The organic extract was dried over anhydrous sodium sulfate, filtered, and concentrated. The resulting residue was purified by flash silica gel chromatography (gradient: 0˜50% EtOAc in petroleum ether) to afford 4-(azepan-1-yl)-7-(8-ethyl-1-naphthyl)-8-fluoro-2-methylsulfonyl-pyrido[4,3-d]pyrimidine (532 mg, 1.11 mmol, 99% yield) as a white solid. LC/MS (ESI) m/z: 479.3 [M+H]⁺. ¹H NMR (400 MHz, DMSO-d₆) δ 9.37 (s, 1H), 8.11 (dd, J=1.2, 8.4 Hz, 1H), 7.95 (s, 2H), 7.65-7.57 (m, 1H), 7.53 (t, J=7.6 Hz, 1H), 7.48 (dd, J=1.2, 7.2 Hz, 1H), 7.42 (d, J=6.8 Hz, 1H), 4.11 (s, 2H), 3.40 (s, 2H), 3.30 (s, 1H), 2.89 (s, 4H), 2.73 (s, 4H), 2.32-2.21 (m, 1H), 1.97 (s, 2H), 1.60 (s, 3H).

Step 3: Preparation of [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methoxy-tert-butyl-diphenyl-silane

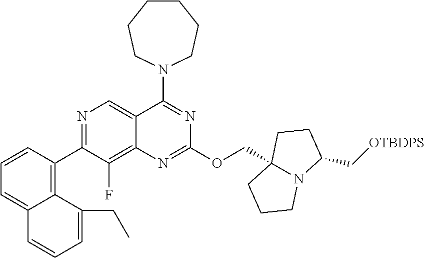

To a solution of 4-(azepan-1-yl)-7-(8-ethyl-1-naphthyl)-8-fluoro-2-methylsulfonyl-pyrido[4,3-d]pyrimidine (500 mg, 1.04 mmol, 1 eq) and [(3R,8S)-3-[[tert-butyl(diphenyl)silyl]oxymethyl]-1,2,3,5,6,7-hexahydropyrrolizin-8-yl]methanol (426 mg, 1.04 mmol, 1 eq) in toluene (10 mL) were added sodium tert-butoxide (300 mg, 3.12 mmol, 3 eq) and 4 A MS (1.04 mmol, 1 eq), and the reaction mixture was stirred at 0° C. for 0.5 h. The pH was adjusted to ˜7 by progressively adding aqueous HCl (2 M). The resulting mixture was diluted with water (20 mL) and extracted with ethyl acetate (20 mL×3). The combined organic extracts were washed brine (10 mL), dried over sodium sulfate, filtered, and concentrated under reduced pressure. The resulting residue was purified by flash silica gel chromatography (gradient: 0˜40% EtOAc in petroleum ether) to afford [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methoxy-tert-butyl-diphenyl-silane (373 mg, 0.46 mmol, 44% yield) as a white solid. LC/MS (ESI) m/z: 808.4 [M+H]⁺.

Step 4: Preparation of [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methanol

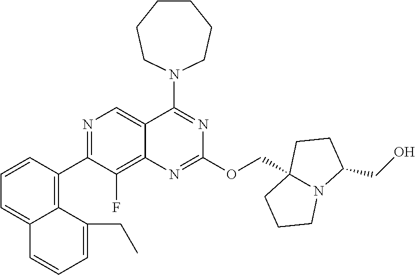 [SMILES: CCc1cccc2cccc(-c3ncc4c(N5CCCCCC5)nc(OC[C@@]56CCCN5[C@@H](CO)CC6)nc4c3F)c12]

To a solution of [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methoxy-tert-butyl-diphenyl-silane (373 mg, 0.46 mmol, 1 eq) in DMF (2 mL) was added CsF (561 mg, 3.69 mmol, 0.1 mL, 8 eq), and the reaction mixture was stirred at 20° C. for 7 h. The mixture was diluted with water (20 mL) and extracted with ethyl acetate (10 mL×3). The combined organic extract was washed with brine (20 mL×2), dried with anhydrous sodium sulfate, filtered and concentrated. The resulting residue was purified by prep-TLC (dichloromethane/methanol=10/1) to afford [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methanol (159 mg, 0.28 mmol, 60% yield) as a white solid. LC/MS (ESI) m/z: 570.3 [M+H]⁺

Step 5: Preparation of [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl (4-nitrophenyl) carbonate

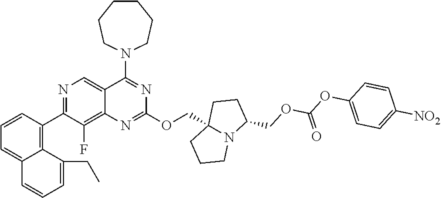 [SMILES: CCc1cccc2cccc(-c3ncc4c(N5CCCCCC5)nc(OC[C@@]56CCCN5[C@@H](COC(=O)Oc5ccc([N+](=O)[O-])cc5)CC6)nc4c3F)c12]

To a solution of [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methanol (159 mg, 0.28 mmol, 1 eq) and (4-nitrophenyl) carbonochloridate (113 mg, 0.56 mmol, 2 eq) in dichloromethane (5 mL) were added dimethylaminopyridine (35 mg, 0.29 mmol, 1.03 eq) and triethylamine (145 mg, 1.44 mmol, 0.2 mL, 5.15 eq), and the reaction mixture was stirred at 25° C. for 6 h. The mixture was concentrated under reduced pressure at 45° C., and the resulting residue was purified by flash silica gel chromatography (gradient: of 0˜100% EtOAc in petroleum ether) to afford [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl (4-nitrophenyl) carbonate (180 mg, 0.25 mmol, 88% yield) as a white solid.

Step 6: Preparation of [(3R,7aR)-7a-{[4-(azepan-1-yl)-7-(8-ethylnaphthalen-1-yl)-8-fluoropyrido[4,3-d]pyrimidin-2-yl]oxy}-hexahydro-1H-pyrrolizin-3-yl]methyl 4-{5-[(2R*)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

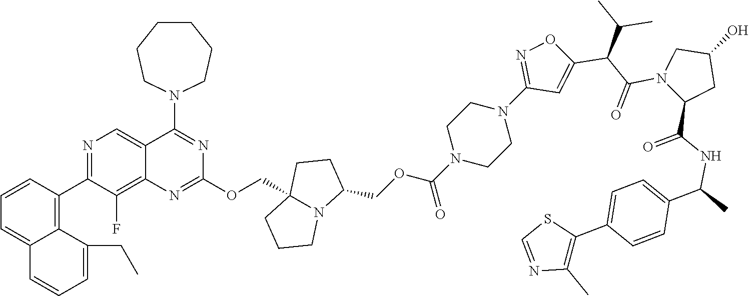 [SMILES: CCc1cccc2cccc(-c3ncc4c(N5CCCCCC5)nc(OC[C@@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a mixture of [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl (4-nitrophenyl) carbonate (180 mg, 0.25 mmol, 1 eq) and (2S,4R)-4-hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (120 mg, 0.2 mmol, 7.82e-1 eq, formate) in dichloromethane (5 mL) was added triethylamine (73 mg, 0.72 mmol, 0.1 mL, 2.87 eq) in one portion at 25° C. under nitrogen, and the reaction mixture was stirred at 25° C. for 6 hours. The residue was poured into water (20 mL) and stirred for 1 minute. The aqueous phase was extracted with dichloromethane (10 mL×3), and the combined organic extract was washed with brine (10 mL×2), dried with anhydrous sodium sulfate, filtered and concentrated. The resulting residue was purified by prep-HPLC (column: Unisil 3-100 C18 Ultra 150*50 mm*3 um; mobile phase: [33-63% CH₃CN in water (formic acid)]) to afford [(3R,7aR)-7a-{[4-(azepan-1-yl)-7-(8-ethylnaphthalen-1-yl)-8-fluoropyrido[4,3-d]pyrimidin-2-yl]oxy}-hexahydro-1H-pyrrolizin-3-yl]methyl 4-{5-[(2R*)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (49.3 mg, 0.04 mmol, 16% yield, 96% purity) as a white solid. LC/MS (ESI) m/z: 1162.4 [M+H]⁺. ¹HNMR (400 MHz, DMSO-d₆) δ 9.14 (s, 1H), 8.98 (s, 1H), 8.40 (d, J=6.8 Hz, 1H), 8.07 (d, J=8.1 Hz, 1H), 7.91 (d, J=8.0 Hz, 1H), 7.57 (dt, J=4.8, 7.6 Hz, 1H), 7.51 (t, J=7.6 Hz, 1H), 7.43 (dd, J=1.4, 6.4 Hz, 3H), 7.37 (t, J=8.2 Hz, 3H), 6.19-6.03 (m, 1H), 4.91 (quin, J=7.1 Hz, 1H), 4.36 (t, J=7.8 Hz, 1H), 4.28 (s, 1H), 4.17-4.09 (m, 1H), 4.06-3.92 (m, 6H), 3.90-3.83 (m, 1H), 3.71 (dd, J=4.0, 10.5 Hz, 1H), 3.62-3.55 (m, 1H), 3.51-3.38 (m, 6H), 3.16 (s, 4H), 2.98-2.89 (m, 2H), 2.54 (s, 3H), 2.45 (s, 3H), 2.32-2.24 (m, 1H), 2.08-1.90 (m, 7H), 1.86-1.74 (m, 4H), 1.71-1.51 (m, 7H), 1.44-1.34 (m, 3H), 0.98-0.92 (m, 3H), 0.84 (t, J=7.4 Hz, 3H), 0.81-0.76 (m, 3H).

Exemplary Synthesis of (2S,4R)-1-[(2R)-2-(3-{[1-(2-{[7-(3-chloro-2-cyclopropyl-5-hydroxyphenyl)-4-{3,8-diazabicyclo[3.2.1]octan-3-yl}-8-fluoropyrido[4,3-d]pyrimidin-2-yl]oxy}ethyl) piperidin-4-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 43)

Step 1: Preparation of 1-bromo-3-chloro-2-cyclopropyl-benzene

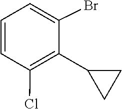 [SMILES: Clc1cccc(Br)c1C1CC1]

To a solution of 1-bromo-3-chloro-2-iodo-benzene (5 g, 15.76 mmol, 1.0 eq) and cyclopropylboronic acid (2.03 g, 23.63 mmol, 1.5 eq) in dioxane (45 mL) and H₂O (15 mL) were added K₃PO₄ (12.04 g, 56.72 mmol, 3.6 eq) and Pd(dppf) Cl₂ (576 mg, 0.788 mmol, 0.05 eq), and the reaction mixture was stirred at 100° C. for 22 hours under N₂. The mixture was concentrated in vacuum, and the resulting residue was dissolved in ethyl acetate. Water (30 mL) was added, and the aqueous phase was extracted with ethyl acetate (50 mL×3). The combined organic extracts was washed with brine (40 mL), dried with anhydrous Na₂SO₄, filtered, and concentrated in vacuum. The residue was purified by flash chromatography on SiO₂ (eluent: 100% petroleum ether) to afford 1-bromo-3-chloro-2-cyclopropyl-benzene (2.15 g, 9.29 mmol, 59% yield) as a colorless oil. ¹H NMR (400 MHz, CDCl₃) δ 7.47 (dd, J=0.8, 8.0 Hz, 1H), 7.31 (dd, J=0.8, 8.0 Hz, 1H), 7.00 (t, J=8.0 Hz, 1H), 1.84-1.72 (m, 1H), 1.23-1.14 (m, 2H), 0.83-0.74 (m, 2H).

Step 2: Preparation of 2-(3-bromo-5-chloro-4-cyclopropyl-phenyl)-4,4,5,5-tetramethyl-1,3,2-dioxaborolane

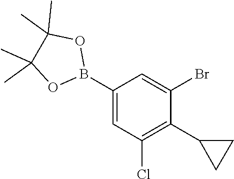 [SMILES: CC1(C)OB(c2cc(Cl)c(C3CC3)c(Br)c2)OC1(C)C]

To a solution of 1-bromo-3-chloro-2-cyclopropyl-benzene (2.6 g, 11.23 mmol, 1.0 eq) and 4,4,5,5-tetramethyl-1,3,2-dioxaborolane (4.31 g, 33.69 mmol, 3.0 eq) in hexane (45 mL) were added (1Z,5Z)-cycloocta-1,5-diene; 2,4-dimethyl-BLAHbicyclo[1.1.0]butane (372 mg, 0.562 mmol, 0.05 eq) and 4-tert-butyl-2-(4-tert-butyl-2-pyridyl)pyridine (181 mg, 0.674 mmol, 0.06 eq), and the reaction mixture was stirred at 60° C. for 7 hours under N₂. The mixture was concentrated in vacuum to give crude 2-(3-bromo-5-chloro-4-cyclopropyl-phenyl)-4,4,5,5-tetramethyl-1,3,2-dioxaborolane (4.01 g) as a black brown oil.

Step 3: Preparation of 3-bromo-5-chloro-4-cyclopropyl-phenol

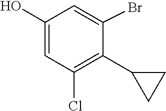 [SMILES: Oc1cc(Cl)c(C2CC2)c(Br)c1]

To a solution of 2-(3-bromo-5-chloro-4-cyclopropyl-phenyl)-4,4,5,5-tetramethyl-1,3,2-dioxaborolane (4.01 g, 11.22 mmol, 1.0 eq) in tetrahydrofuran (40 mL) and H₂O (20 mL) were added acetic acid (720.16 mmol, 41.19 mL, 64.2 eq) and H₂O₂ (213.13 mmol, 20.48 mL, 30% purity, 19.0 eq), and the reaction mixture was stirred at 0° C. for 1.5 hours. Water (20 mL) was added, and the aqueous phase was extracted with ethyl acetate (50 mL×3). The combined organic extracts was washed with H₂O (20 mL) and saturated aqueous Na₂SO₃, dried over anhydrous Na₂SO₄, filtered, and concentrated in vacuum. The residue was purified by flash chromatography on SiO₂ (gradient: 0˜3% ethyl acetate in petroleum ether) to afford 3-bromo-5-chloro-4-cyclopropyl-phenol (2.7 g, 9.82 mmol, 88% yield) as a yellow gum. ¹H NMR (400 MHz, CDCl₃) δ 7.01 (d, J=2.4 Hz, 1H), 6.85 (d, J=2.4 Hz, 1H), 5.13-4.66 (m, 1H), 1.70-1.66 (m, 1H), 1.16-1.10 (m, 2H), 0.74-0.69 (m, 2H).

Step 4: Preparation of 1-bromo-3-chloro-2-cyclopropyl-5-(methoxymethoxy)benzene

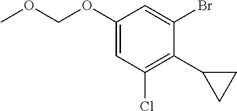

To a solution of 3-bromo-5-chloro-4-cyclopropyl-phenol (2.7 g, 10.91 mmol, 1.0 eq) in dichloromethane (30 mL) at 0° C. were added DIEA (43.63 mmol, 7.60 mL, 4.0 eq) and MOMCl (1.76 g, 21.82 mmol, 2.0 eq) dropwise, and the reaction mixture was stirred at 20° C. for 1 hour. The reaction was quenched by addition of water (40 mL) at 0° C., then extracted with dichloromethane (50 mL×3). The combined organic extracts was washed with brine (50 mL), dried over anhydrous sodium sulfate, filtered, and concentrated in vacuum. The resulting residue was purified by flash chromatography on SiO₂ (gradient: 0˜1% ethyl acetate in petroleum ether) to afford 1-bromo-3-chloro-2-cyclopropyl-5-(methoxymethoxy)benzene (2.5 g, 8.57 mmol, 79% yield) as a colorless oil. ¹H NMR (400 MHz, CDCl₃) δ 7.20 (d, J=2.4 Hz, 1H), 7.04 (d, J=2.4 Hz, 1H), 5.12 (s, 2H), 3.47 (s, 3H), 1.70 (tt, J=5.6, 8.4 Hz, 1H), 1.17-1.11 (m, 2H), 0.75-0.70 (m, 2H).

Step 5: Preparation of 2-[3-chloro-2-cyclopropyl-5-(methoxymethoxy)phenyl]-4,4,5,5-tetramethyl-1,3,2-dioxaborolane

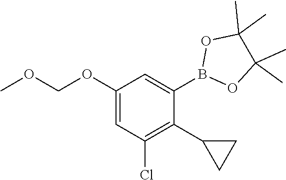

To a solution of 1-bromo-3-chloro-2-cyclopropyl-5-(methoxymethoxy)benzene (500 mg, 1.71 mmol, 1.0 eq) and 4,4,5,5-tetramethyl-2-(4,4,5,5-tetramethyl-1,3,2-dioxaborolan-2-yl)-1,3,2-dioxaborolane (871 mg, 3.43 mmol, 2.0 eq) in dioxane (6 mL) were added KOAc (505 mg, 5.14 mmol, 3.0 eq) and Pd(dppf) Cl₂ (125 mg, 0.171 mmol, 0.1 eq), and the reaction mixture was stirred at 100° C. for 12 hours under N₂. The residue was dissolved in ethyl acetate. Water (30 mL) was then added, and the aqueous phase was extracted with ethyl acetate (40 mL×3). The combined organic extracts were washed with brine (30 mL), dried with anhydrous Na₂SO₄, filtered, and concentrated in vacuum. The residue was purified by flash chromatography on SiO₂ (gradient: 0˜1% ethyl acetate in petroleum ether) to afford 2-[3-chloro-2-cyclopropyl-5-(methoxymethoxy)phenyl]-4,4,5,5-tetramethyl-1,3,2-dioxaborolane (395 mg, 1.17 mmol, 68% yield) as a colorless oil. ¹H NMR (400 MHz, CDCl₃) δ 7.12-7.08 (m, 2H), 5.14 (s, 2H), 3.46 (s, 3H), 2.01-1.94 (m, 1H), 1.38 (s, 12H), 1.01-0.96 (m, 2H), 0.55-0.50 (m, 2H).

Step 6: Preparation of tert-butyl 3-[7-[3-chloro-2-cyclopropyl-5-(methoxymethoxy)phenyl]-2-(2,2-dimethoxyethoxy)-8-fluoro-pyrido[4,3-d]pyrimidin-4-yl]-3,8-diazabicyclo[3.2.1]octane-8-carboxylate

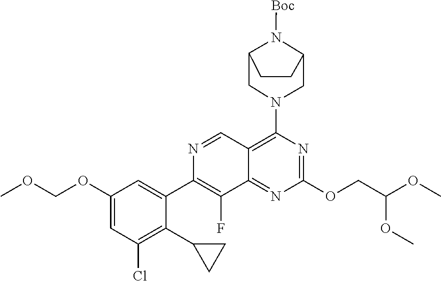

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (380 mg, 0.763 mmol, 1.0 eq) and 2-[3-chloro-2-cyclopropyl-5-(methoxymethoxy)phenyl]-4,4,5,5-tetramethyl-1,3,2-dioxaborolane (388 mg, 1.14 mmol, 1.5 eq) in dioxane (7.5 mL) and H₂O (1.5 mL) were added K₃PO₄ (486 mg, 2.29 mmol, 3.0 eq) and [2-(2-aminophenyl)phenyl]palladium (1+); bis(1-adamantyl)-butyl-phosphane; methanesulfonate (56 mg, 0.076 mmol, 0.1 eq) in one portion at 25° C., and the reaction mixture was stirred at 8 5° C. for 12 hours under N₂. The residue was dissolved in ethyl acetate. Water (30 mL) was then added, and the aqueous phase was extracted with ethyl acetate (50 mL×3). The combined organic extract was washed with brine (40 mL), dried over anhydrous Na₂SO₄, filtered, and concentrated in vacuum. The residue was purified by flash chromatography on SiO₂ (gradient: 0˜28% ethyl acetate in petroleum ether) to afford tert-butyl 3-[7-[3-chloro-2-cyclopropyl-5-(methoxymethoxy)phenyl]-2-(2,2-dimethoxyethoxy)-8-fluoro-pyrido[4,3-d]pyrimidin-4-yl]-3,8-diazabicyclo[3.2.1]octane-8-carboxylate (442 mg, 0.538 mmol, 70% yield) as a yellow solid. LC/MS (ESI) m/z: 674.2 [M+H]⁺.

Step 7: Preparation of tert-butyl 3-[7-(3-chloro-2-cyclopropyl-5-hydroxy-phenyl)-8-fluoro-2-(2-oxoethoxy)pyrido[4,3-d]pyrimidin-4-yl]-3,8-diazabicyclo[3.2.1]octane-8-carboxylate

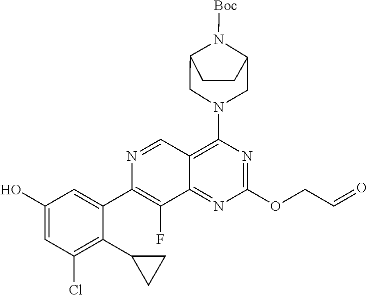 [SMILES: CC(C)(C)OC(=O)N1C2CCC1CN(c1nc(OCC=O)nc3c(F)c(-c4cc(O)cc(Cl)c4C4CC4)ncc13)C2]

To a solution of tert-butyl 3-[7-[3-chloro-2-cyclopropyl-5-(methoxymethoxy)phenyl]-2-(2,2-dimethoxyethoxy)-8-fluoro-pyrido[4,3-d]pyrimidin-4-yl]-3,8-diazabicyclo[3.2.1]octane-8-carboxylate (390 mg, 0.579 mmol, 1.0 eq) in acetone (1.1 mL) was added aqueous HCl (12 M, 868 μL, 18.0 eq), and the reaction mixture was stirred at 20° C. for 15 minutes. The mixture was poured onto a solution of NaHCO₃ (1.94 g, 23.14 mmol, 40 eq) in water (6 mL) and tetrahydrofuran (2 mL). Boc₂O (0.868 mmol, 199 μL, 1.5 eq) was then added, and the reaction mixture was stirred at 20° C. for 0.5 hour. Water (10 mL) was added, and the aqueous phase was extracted with ethyl acetate (30 mL×3). The combined organic extract was washed with brine (20 mL), dried over anhydrous Na₂SO₄, filtered, and concentrated in vacuum. The residue was purified by flash chromatography on SiO₂ (gradient: 0˜51% ethyl acetate in petroleum ether) to afford tert-butyl 3-[7-(3-chloro-2-cyclopropyl-5-hydroxy-phenyl)-8-fluoro-2-(2-oxoethoxy)pyrido[4,3-d]pyrimidin-4-yl]-3,8-diazabicyclo[3.2.1]octane-8-carboxylate (253 mg, 0.321 mmol, 55% yield) as a white solid. LC/MS (ESI) m/z: 584.2 [M+H]⁺.

Step 8: Preparation of (2S,4R)-1-[(2R)-2-(3-{[1-(2-{[7-(3-chloro-2-cyclopropyl-5-hydroxyphenyl)-4-{3,8-diazabicyclo[3.2.1]octan-3-yl}-8-fluoropyrido[4,3-d]pyrimidin-2-yl]oxy}ethyl) piperidin-4-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide

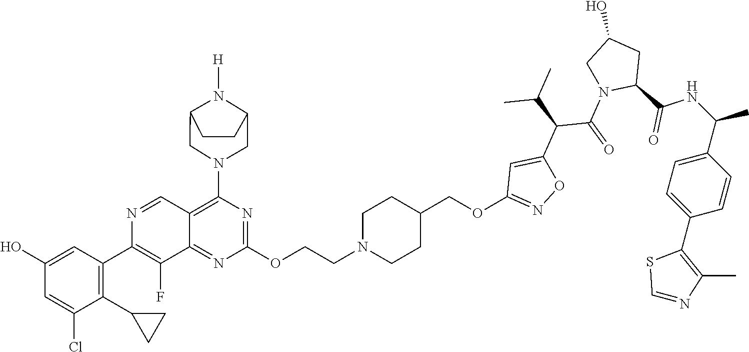 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc(Cl)c6C6CC6)c(F)c5n4)CC3)no2)C(C)C)cc1]

The title compound was prepared in an analogous manner to Compound 20 starting from tert-butyl 3-[7-(3-chloro-2-cyclopropyl-5-hydroxy-phenyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide, and purified by prep-HPLC (column: Phenomenex C18 75*30 mm*3 um; mobile phase: [5-45% CH₃CN in water (FA)]). The pure fractions were combined, then lyophilized to afford (2S,4R)-1-[(2R)-2-(3-{[1-(2-{[7-(3-chloro-2-cyclopropyl-5-hydroxyphenyl)-4-{3,8-diazabicyclo[3.2.1]octan-3-yl}-8-fluoropyrido[4,3-d]pyrimidin-2-yl]oxy}ethyl) piperidin-4-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (78.7 mg, 0.072 mmol, 60% yield, FA) as a white solid. LC/MS (ESI) m/z: 1063.7 [M+H]⁺. ¹H NMR (400 MHz, CD₃OD) δ 9.10 (s, 1H), 8.88 (s, 1H), 8.44 (s, 1H), 7.48-7.36 (m, 4H), 7.00-6.95 (m, 1H), 6.79 (d, J=2.4 Hz, 1H), 5.99-5.93 (m, 1H), 5.08-4.98 (m, 1H), 4.74 (dd, J=4.4, 10.4 Hz, 3H), 4.69 (s, 1H), 4.50 (t, J=8.4 Hz, 1H), 4.46-4.37 (m, 1H), 4.09 (d, J=6.0 Hz, 2H), 3.92 (s, 2H), 3.87-3.77 (m, 3H), 3.68 (d, J=10.0 Hz, 1H), 3.61 (d, J=10.9 Hz, 1H), 3.48-3.42 (m, 2H), 3.28-3.22 (m, 2H), 2.76-2.62 (m, 2H), 2.50-2.46 (m, 3H), 2.41-2.32 (m, 1H), 2.24-2.13 (m, 1H), 2.04-1.91 (m, 8H), 1.88-1.81 (m, 1H), 1.65-1.55 (m, 2H), 1.55-1.49 (m, 3H), 1.05 (d, J=6.4 Hz, 3H), 0.94-0.86 (m, 3H), 0.70-0.53 (m, 2H), 0.07 (d, J=3.2 Hz, 2H).

Exemplary Synthesis of (2S,4R)-1-[(2R)-2-[3-({1-[2-({7-[3-chloro-5-hydroxy-2-(propan-2-yl)phenyl]-4-{3,8-diazabicyclo[3.2.1]octan-3-yl}-8-fluoropyrido[4,3-d]pyrimidin-2-yl}oxy)ethyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 41)

Step 1: Preparation of 1-bromo-3-chloro-2-isopropenyl-benzene

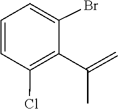

To a solution of 1-bromo-3-chloro-2-iodo-benzene (5.0 g, 15.76 mmol, 1.0 eq) and 2-isopropenyl-4,4,5,5-tetramethyl-1,3,2-dioxaborolane (3.4 g, 20.48 mmol, 1.3 eq) in dioxane (75 mL) and H₂O (25 mL) were added K₃PO₄ (10.0 g, 47.27 mmol, 3.0 eq) and Pd(dppf) Cl₂ (576 mg, 0.788 mmol, 0.05 eq), and the reaction mixture was stirred at 90° C. for 20 hours under N₂. The reaction mixture was concentrated, and the resulting was dissolved in ethyl acetate. Water (30 mL) was added, and the aqueous phase was extracted with ethyl acetate (50 mL×3). The combined organic extract was washed with brine (40 mL), dried with anhydrous Na₂SO₄, filtered, and concentrated in vacuum. The residue was purified by flash chromatography on SiO₂ (gradient: 0˜0% ethyl acetate in petroleum ether) to afford 1-bromo-3-chloro-2-isopropenyl-benzene (2.7 g, 11.84 mmol, 75% yield) as a yellow oil. ¹H NMR (400 MHz, CDCl₃) δ 7.50 (dd, J=0.8, 8.0 Hz, 1H), 7.35 (dd, J=0.8, 8.0 Hz, 1H), 7.06 (t, J=8.0 Hz, 1H), 5.39-5.31 (m, 1H), 4.92 (s, 1H), 2.05-2.03 (m, 3H).

Step 2: Preparation of 1-bromo-3-chloro-2-isopropyl-benzene

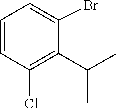

To a solution of 1-bromo-3-chloro-2-isopropenyl-benzene (2.7 g, 11.84 mmol, 1.0 eq) in ethyl acetate (30 mL) was added PtO₂ (1.0 g, 4.73 mmol, 0.4 eq), and the resulting mixture was degassed under vacuum and purged with H₂ (23.9 mg, 11.84 mmol, 1.0 eq) several times. The reaction mixture was stirred under H₂ (15 psi) at 25° C. for 18 hours. The mixture was filtered, then concentrated in vacuum to afford 1-bromo-3-chloro-2-isopropyl-benzene (2.6 g, 10.96 mmol, 93% yield) as a yellow oil. ¹H NMR (400 MHz, CD₃OD) δ 7.54 (d, J=7.2 Hz, 1H), 7.40-7.29 (m, 1H), 7.03 (t, J=8.0 Hz, 1H), 3.92 (d, J=5.2 Hz, 1H), 1.42 (d, J=7.2 Hz, 6H).

Step 3: Preparation of 2-(3-bromo-5-chloro-4-isopropyl-phenyl)-4,4,5,5-tetramethyl-1,3,2-dioxaborolane

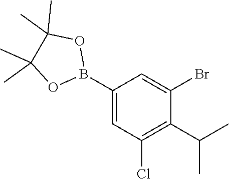 [SMILES: CC(C)c1c(Cl)cc(B2OC(C)(C)C(C)(C)O2)cc1Br]

To a solution of 1-bromo-3-chloro-2-isopropyl-benzene (3.3 g, 14.30 mmol, 1.0 eq) and 4,4,5,5-tetramethyl-1,3,2-dioxaborolane (5.5 g, 42.91 mmol, 3 eq) in hexane (80 mL) were added (1Z,5Z)-cycloocta-1,5-diene; 2,4-dimethyl-BLAHbicyclo[1.1.0]butane (569 mg, 0.858 mmol, 0.06 eq) and 4-tert-butyl-2-(4-tert-butyl-2-pyridyl)pyridine (269 mg, 1.00 mmol, 0.07 eq), and the reaction mixture was stirred at 70° C. for 10 hours under N₂. The reaction mixture was concentrated in vacuum to afford 2-(3-bromo-5-chloro-4-isopropyl-phenyl)-4,4,5,5-tetramethyl-1,3,2-dioxaborolane (5.1 g, crude) as a black brown oil.

Step 4: Preparation of 3-bromo-5-chloro-4-isopropyl-phenol

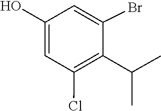

To a solution of 2-(3-bromo-5-chloro-4-isopropyl-phenyl)-4,4,5,5-tetramethyl-1,3,2-dioxaborolane (5.1 g, 14.19 mmol, 1.0 eq) in tetrahydrofuran (48 mL) and H₂O (24 mL) were added acetic acid 51.1 g, 851.20 mmol, 48.68 mL, 60.0 eq) and H₂O₂ (113.49 mmol, 10.90 mL, 30% purity, 8.0 eq), and the reaction mixture was stirred at 0° C. for 4 hours. Additional H₂O₂ (113.49 mmol, 10.91 mL, 30% purity, 8.0 eq) was added, and the reaction mixture was stirred at 0° C. for 3 hours. Water (30 mL) was added, and the aqueous phase was extracted with ethyl acetate (60 mL×3). The combined organic extract was washed with H₂O (20 mL×2) and saturated aqueous Na₂SO₃ (20 mL×5), dried with anhydrous Na₂SO₄, filtered, and concentrated in vacuum. The residue was purified by flash chromatography on SiO₂ (gradient: 0˜5% ethyl acetate in petroleum ether) to afford 3-bromo-5-chloro-4-isopropyl-phenol (1.4 g, 5.61 mmol, 40% yield) as a colorless oil. ¹H NMR (400 MHz, CDCl₃) δ 7.02 (d, J=2.4 Hz, 1H), 6.84 (s, 1H), 3.85-3.70 (m, 1H), 1.39 (d, J=7.2 Hz, 6H).

Step 5: Preparation of 1-bromo-3-chloro-2-isopropyl-5-(methoxymethoxy)benzene

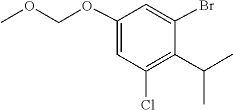

To a solution of 3-bromo-5-chloro-4-isopropyl-phenol (1.2 g, 4.77 mmol, 1.0 eq) in dichloromethane (24 mL) at 0° C. were added DIEA (11.92 mmol, 2.08 mL, 2.5 eq) and MOMCl (12.88 mmol, 978 μL, 2.7 eq) dropwise, and the reaction mixture was stirred at 0° C. for 0.5 hour. The reaction was quenched by addition of water (30 mL) at 0° C., then extracted with dichloromethane (40 ml×3). The combined organic extract was washed with brine (30 mL), dried over anhydrous sodium sulfate, filtered, and concentrated. The resulting residue was purified by flash chromatography on SiO₂ (gradient: 0˜5% ethyl acetate in petroleum ether) to afford 1-bromo-3-chloro-2-isopropyl-5-(methoxymethoxy)benzene (1.3 g, 4.26 mmol, 89% yield) as a colorless oil. ¹H NMR (400 MHz, DMSO-d₆) δ 7.28 (s, 1H), 7.13 (s, 1H), 5.21 (s, 2H), 3.73 (d, J=1.2 Hz, 1H), 3.36 (s, 3H), 1.34 (d, J=7.2 Hz, 6H).

Step 6: Preparation of 2-[3-chloro-2-isopropyl-5-(methoxymethoxy)phenyl]-4,4,5,5-tetramethyl-1,3,2-dioxaborolane

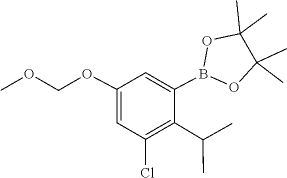

To a solution of 1-bromo-3-chloro-2-isopropyl-5-(methoxymethoxy)benzene (500 mg, 1.70 mmol, 1.0 eq) and 4,4,5,5-tetramethyl-2-(4,4,5,5-tetramethyl-1,3,2-dioxaborolan-2-yl)-1,3,2-dioxaborolane (865 mg, 3.41 mmol, 2.0 eq) in dioxane (6 mL) were added KOAc (501 mg, 5.11 mmol, 3.0 eq) and Pd(dppf) Cl₂ (125 mg, 0.170 mmol, 0.1 eq), and the reaction mixture was stirred at 100° C. for 12 hours under N₂. The residue was dissolved in ethyl acetate. Water (25 mL) was added, and the aqueous phase was extracted with ethyl acetate (30 mL×3). The combined organic extract was washed with brine (30 mL), dried with anhydrous Na₂SO₄, filtered, and concentrated in vacuum. The residue was purified by flash chromatography on SiO₂ (gradient: 0˜2% ethyl acetate in petroleum ether) to afford 2-[3-chloro-2-isopropyl-5-(methoxymethoxy)phenyl]-4,4,5,5-tetramethyl-1,3,2-dioxaborolane (307 mg, 0.901 mmol, 53% yield) as a yellow oil. ¹H NMR (400 MHz, CDCl₃) δ 7.13 (d, J=2.8 Hz, 1H), 7.09 (d, J=2.8 Hz, 1H), 5.14 (s, 2H), 3.67-3.60 (m, 1H), 3.46 (s, 3H), 1.38 (s, 3H), 1.37 (s, 12H), 1.36 (s, 3H).

Step 7: Preparation of tert-butyl 3-[7-[3-chloro-2-isopropyl-5-(methoxymethoxy)phenyl]-2-(2,2-dimethoxyethoxy)-8-fluoro-pyrido[4,3-d]pyrimidin-4-yl]-3,8-diazabicyclo[3.2.1]octane-8-carboxylate

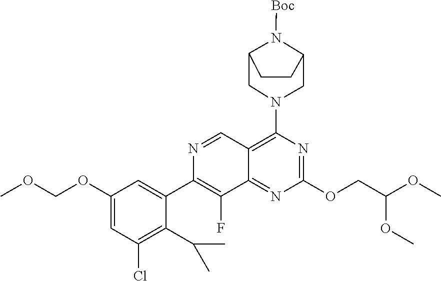

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (299 mg, 0.600 mmol, 1.0 eq) and 2-[3-chloro-2-isopropyl-5-(methoxymethoxy)phenyl]-4,4,5,5-tetramethyl-1,3,2-dioxaborolane (307 mg, 0.901 mmol, 1.5 eq) in dioxane (6 mL) and H₂O (1.2 mL) at 20° C. mg, were added K₃PO₄ (382 1.80 mmol, 3.0 eq) and [2-(2-aminophenyl)phenyl]palladium (1+); bis(1-adamantyl)-butyl-phosphane; methanesulfonate (44 mg, 0.060 mmol, 0.1 eq) in one portion, and the reaction mixture was stirred at 85° C. for 18 hours under N₂. Ethyl acetate was added followed by water (30 mL), and the aqueous phase was extracted with ethyl acetate (40 mL×3). The combined organic extract was washed with brine (30 mL), dried with anhydrous Na₂SO₄, filtered, and concentrated in vacuum. The residue was purified by flash chromatography on SiO₂ (gradient: 0˜25% ethyl acetate in petroleum ether) to afford tert-butyl 3-[7-[3-chloro-2-isopropyl-5-(methoxymethoxy)phenyl]-2-(2,2-dimethoxyethoxy)-8-fluoro-pyrido[4,3-d]pyrimidin-4-yl]-3,8-diazabicyclo[3.2.1]octane-8-carboxylate (428 mg, 0.513 mmol, 85% yield) as a yellow oil. LC/MS (ESI) m/z: 676.2 [M+H]⁺.

Step 8: Preparation of tert-butyl 3-[7-(3-chloro-5-hydroxy-2-isopropyl-phenyl)-8-fluoro-2-(2-oxoethoxy)pyrido[4,3-d]pyrimidin-4-yl]-3,8-diazabicyclo[3.2.1]octane-8-carboxylate

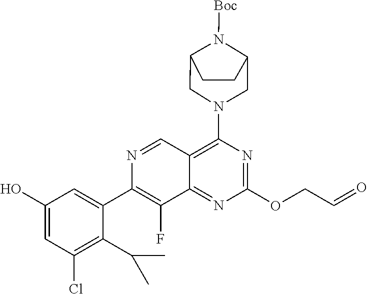 [SMILES: CC(C)c1c(Cl)cc(O)cc1-c1ncc2c(N3CC4CCC(C3)N4C(=O)OC(C)(C)C)nc(OCC=O)nc2c1F]

To a solution of tert-butyl 3-[7-[3-chloro-2-isopropyl-5-(methoxymethoxy)phenyl]-2-(2,2-dimethoxyethoxy)-8-fluoro-pyrido[4,3-d]pyrimidin-4-yl]-3,8-diazabicyclo[3.2.1]octane-8-carboxylate (378 mg, 0.559 mmol, 1.0 eq) in acetone (840 uL) was added aqueous HCl (12 M, 839 μL, 18.0 eq), and the reaction mixture was stirred at 20° C. for 15 minutes. The mixture were poured onto a solution of NaHCO₃ (1.9 g, 22.36 mmol, 40.0 eq) in H₂O (3 mL) and tetrahydrofuran (4 mL). Boc₂₀ (0.839 mmol, 193 μL, 1.5 eq) was added, and the reaction mixture was stirred at 20° C. for 0.5 hours. Water (10 mL) was added, and the aqueous phase was extracted with ethyl acetate (20 mL×3). The combined organic extract was washed with brine (10 mL), dried with anhydrous Na₂SO₄, filtered, and concentrated in vacuum. The residue was purified by flash chromatography on SiO₂ (gradient: 0˜39% ethyl acetate in petroleum ether) to afford tert-butyl 3-[7-(3-chloro-5-hydroxy-2-isopropyl-phenyl)-8-fluoro-2-(2-oxoethoxy)pyrido[4,3-d]pyrimidin-4-yl]-3,8-diazabicyclo[3.2.1]octane-8-carboxylate (170 mg, 229.16 umol, 41% yield) as a yellow solid. LC/MS (ESI) m/z: 586.1 [M+H]⁺.

Step 9: Preparation of (2S,4R)-1-[(2R)-2-[3-({1-[2-({7-[3-chloro-5-hydroxy-2-(propan-2-yl)phenyl]-4-{3,8-diazabicyclo[3.2.1]octan-3-yl}-8-fluoropyrido[4,3-d]pyrimidin-2-yl}oxy)ethyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide

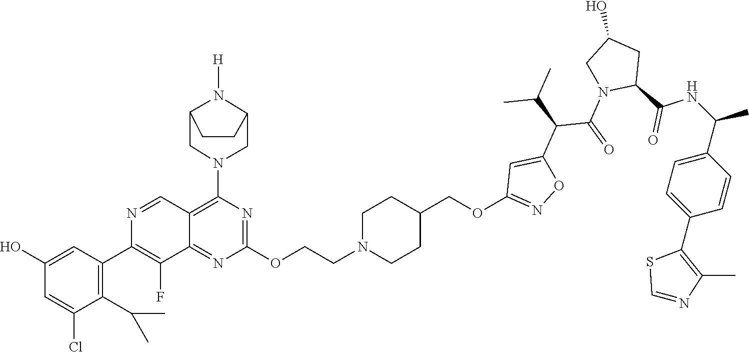 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc(Cl)c6C(C)C)c(F)c5n4)CC3)no2)C(C)C)cc1]

The title compound was prepared in an analogous manner to Compound 20 starting from tert-butyl 3-[7-(3-chloro-5-hydroxy-2-isopropyl-phenyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide, and purified by prep-HPLC (column: Phenomenex C18 75*30 mm*3 um; mobile phase: [15-45% CH₃CN in water (FA)]). The pure fractions were combined, then lyophilized to afford (2S,4R)-1-[(2R)-2-[3-({1-[2-({7-[3-chloro-5-hydroxy-2-(propan-2-yl)phenyl]-4-{3,8-diazabicyclo[3.2.1]octan-3-yl}-8-fluoropyrido[4,3-d]pyrimidin-2-yl}oxy)ethyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (79 mg, 0.070 mmol, 57% yield, FA) as a white solid. LC/MS (ESI) m/z: 1065.8 [M+H]⁺. ¹H NMR (400 MHz, CD₃OD) δ 9.08 (s, 1H), 8.88 (s, 1H), 8.47 (s, 2H), 7.46-7.37 (m, 4H), 6.95 (d, J=2.4 Hz, 1H), 6.61 (d, J=2.4 Hz, 1H), 6.09-5.89 (m, 1H), 5.06-4.99 (m, 1H), 4.81-4.76 (m, 3H), 4.51 (t, J=8.0 Hz, 1H), 4.46-4.38 (m, 1H), 4.10 (d, J=6.0 Hz, 2H), 4.05 (s, 2H), 3.91-3.80 (m, 3H), 3.68 (d, J=9.6 Hz, 1H), 3.64-3.52 (m, 3H), 3.37 (s, 2H), 3.03-2.90 (m, 1H), 2.89-2.78 (m, 2H), 2.50-2.45 (m, 3H), 2.40-2.32 (m, 1H), 2.19 (dd, J=8.0, 13.2 Hz, 1H), 2.12-1.91 (m, 9H), 1.66-1.50 (m, 5H), 1.26 (d, J=6.8 Hz, 6H), 1.05 (d, J=6.4 Hz, 3H), 0.92-0.86 (m, 3H).

Exemplary Synthesis of (2S,4R)-1-[(2R)-2-(3-{[1-(2-{[7-(8-chloro-3-hydroxynaphthalen-1-yl)-4-{3,8-diazabicyclo[3.2.1]octan-3-yl}-8-fluoropyrido[4,3-d]pyrimidin-2-yl]oxy}ethyl) piperidin-4-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 40)

Step 1: Preparation of 2,4-dibromo-5-chloro-naphthalen-1-amine

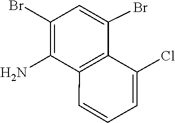 [SMILES: Nc1c(Br)cc(Br)c2c(Cl)cccc12]

To a solution of 5-chloronaphthalen-1-amine (4.0 g, 22.52 mmol, 1.0 eq) in CHCl₃ (60 mL) was added bromine (4.64 mL, 90.07 mmol, 4.0 eq) in CHCl₃ (60 mL) dropwise, and the reaction mixture was heated at 50° C. for 15 hours. The reaction mixture was cooled and concentrated under reduced pressure. The residue was dissolved with water (50 mL) and extracted with EtOAc (50 mL×3). The organic extracts were combined and washed with brine (20 mL×2), dried over Na₂SO₄, filtered, and concentrated under reduced pressure. The residue was purified by flash chromatography on SiO₂ (gradient: 0-5% ethyl acetate in petroleum ether) to afford 2,4-dibromo-5-chloro-naphthalen-1-amine (6.16 g, 17.63 mmol, 78% yield) as a dark brown solid. LC/MS (ESI) m/z: 335.7 [M+H]⁺. ¹H NMR (400 MHz, DMSO-d₆) δ 8.31 (dd, J=0.8, 8.4 Hz, 1H), 7.91 (s, 1H), 7.73 (dd, J=0.8, 7.2 Hz, 1H), 7.46 (dd, J=7.8, 8.4 Hz, 1H), 6.29 (br s, 2H).

Step 2: Preparation of 5-bromo-6-chloro-benzo[e][1,2,3]benzoxadiazole

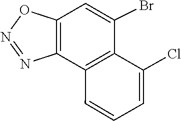 [SMILES: Clc1cccc2c1c(Br)cc1onnc12]

To a mixture of 2,4-dibromo-5-chloro-naphthalen-1-amine (6.16 g, 17.63 mmol, 96% purity, 1.0 eq) and propionic acid (11 mL, 147.32 mmol, 8.36 eq) in acetic acid (110 mL) at 0° C. was added NaNO₂ (1.83 g, 26.46 mmol, 1.5 eq), and the resulting suspension was stirred at 0° C. for 1 hour, then at 25° C. for 1 hour. The mixture was diluted with water (120 mL) and EtOAc (120 mL). The organic layer was separated, and the aqueous layer was further extracted with EtOAc (120 mL×2). The combined organic extracts were washed with brine (100 mL×3), dried over Na₂SO₄, filtered, and concentrated under reduced pressure. The residue was purified by flash chromatography on SiO₂ (gradient: 0˜30% ethyl acetate in petroleum ether) to afford 5-bromo-6-chloro-benzo[e][1,2,3]benzoxadiazole (2.89 g, 9.38 mmol, 53% yield) as a yellow solid. LC/MS (ESI) m/z: 284.8 [M+H]⁺.

Step 3: Preparation of 4-bromo-5-chloro-naphthalen-2-ol

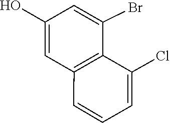 [SMILES: Oc1cc(Br)c2c(Cl)cccc2c1]

To 5-bromo-6-chloro-benzo[e][1,2,3]benzoxadiazole (2.89 g, 9.38 mmol, 1.0 eq) in THF (150 mL) and EtOH (150 mL) at 0° C. was added NaBH₄ (800 mg, 21.15 mmol, 2.25 eq), and the resulting suspension was stirred at 25° C. for 2 hours. The reaction mixture was quenched with water (100 mL) and concentrated under reduced pressure. The residue was adjusted to pH ˜6 by adding aqueous HCl (2 M), and the resulting aqueous mixture was extracted with EtOAc (100 mL×3). The organic extracts were combined, washed with brine (50 mL×3), dried over Na₂SO₄, filtered, and concentrated under reduced pressure. The residue was purified by flash chromatography on SiO₂ (gradient: 0-20% ethyl acetate in petroleum ether) to afford 4-bromo-5-chloro-naphthalen-2-ol (1.38 g, 4.82 mmol, 51% yield) as a brown solid. LC/MS (ESI) m/z: 258.8 [M+H]⁺. ¹H NMR (400 MHz, CDCl₃) δ 7.63-7.57 (m, 2H), 7.49 (dd, J=1.2, 7.6 Hz, 1H), 7.30 (t, J=8.0 Hz, 1H), 7.16 (d, J=2.4 Hz, 1H).

Step 4: Preparation of 1-bromo-8-chloro-3-(methoxymethoxy) naphthalene

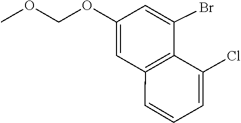 [SMILES: COCOc1cc(Br)c2c(Cl)cccc2c1]

To a mixture of 4-bromo-5-chloro-naphthalen-2-ol (1.38 g, 4.82 mmol, 90% purity, 1.0 eq) and DIEA (2.5 mL, 14.47 mmol, 3.0 eq) in CH₂Cl₂ (24 mL) at 0° C. was added MOMCl (550 uL, 7.24 mmol, 1.5 eq), and the resulting suspension was stirred at 25° C. for 1 hour. The reaction mixture was quenched with water (10 mL). The organic layer was separated, and the aqueous layer was further extracted with CH₂Cl₂ (30 mL×2). The organic extracts were combined, dried over Na₂SO₄, filtered, and concentrated under reduced pressure. The residue was purified by flash chromatography on SiO₂ (gradient: 0% ethyl acetate in petroleum ether) to afford 1-bromo-8-chloro-3-(methoxymethoxy) naphthalene (1.16 g, 3.73 mmol, 77% yield) as a pink solid. ¹H NMR (400 MHz, CDCl₃) δ 7.69 (d, J=2.4 Hz, 1H), 7.67 (dd, J=0.8, 8.4 Hz, 1H), 7.51 (dd, J=1.2, 7.6 Hz, 1H), 7.38 (d, J=2.4 Hz, 1H), 7.34-7.28 (m, 1H), 5.28 (s, 2H), 3.52 (s, 3H).

Step 5: Preparation of 2-[8-chloro-3-(methoxymethoxy)-1-naphthyl]-4,4,5,5-tetramethyl-1,3,2-dioxaborolane

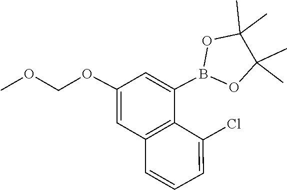

To 1-bromo-8-chloro-3-(methoxymethoxy) naphthalene (1.16 g, 3.73 mmol, 97% purity, 1.0 eq) in dioxane (20 mL) were added 4,4,5,5-tetramethyl-2-(4,4,5,5-tetramethyl-1,3,2-dioxaborolan-2-yl)-1,3,2-dioxaborolane (1.89 g, 7.46 mmol, 2.0 eq), KOAc (1.10 g, 11.19 mmol, 3.0 eq), and Pd(dppf) Cl₂ (273 mg, 373.10 umol, 0.1 eq) under N₂ atmosphere, and the resulting suspension was stirred at 95° C. for 15 hours. The reaction was cooled and diluted with EtOAc (20 mL) and water (20 mL). The mixture was filtered, and the cake was washed with EtOAc (20 mL). The organic layer was separated, and the aqueous layer was further extracted with EtOAc (30 mL×2). The combined organic extracts were washed with brine (30 mL×3), dried over Na₂SO₄, filtered, and concentrated under reduced pressure. The residue was purified by flash chromatography on SiO₂ (gradient: 0-5% ethyl acetate in petroleum ether) followed by flash chromatography on SiO₂ (gradient: 0-3% ethyl acetate in petroleum ether) to afford 2-[8-chloro-3-(methoxymethoxy)-1-naphthyl]-4,4,5,5-tetramethyl-1,3,2-dioxaborolane (879 mg, 2.40 mmol, 64% yield) as an off-white solid. LC/MS (ESI) m/z: 349.0 [M+H]⁺. ¹H NMR (400 MHz, CDCl₃) δ 7.65 (d, J=8.0 Hz, 1H), 7.44 (dd, J=0.8, 7.6 Hz, 1H), 7.42-7.35 (m, 2H), 7.35-7.29 (m, 1H), 5.30 (s, 2H), 3.51 (s, 3H), 1.45 (s, 12H).

Step 6: Preparation of tert-butyl 3-[7-[8-chloro-3-(methoxymethoxy)-1-naphthyl]-2-(2,2-dimethoxyethoxy)-8-fluoro-pyrido[4,3-d]pyrimidin-4-yl]-3,8-diazabicyclo[3.2.1]octane-8-carboxylate

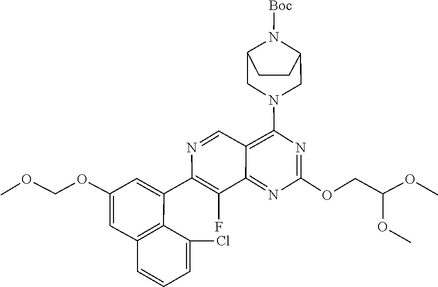

To a mixture of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (320 mg, 642.64 umol, 1.0 eq) and 2-[8-chloro-3-(methoxymethoxy)-1-naphthyl]-4,4,5,5-tetramethyl-1,3,2-dioxaborolane (336.06 mg, 963.96 umol, 1.5 eq) in dioxane (7 mL) were added K₃PO₄ (1.5 M, 1.29 mL, 3.0 [2-(2-aminophenyl)phenyl]palladium (1+); bis(1-adamantyl)-butyl-eq) and phosphane; methanesulfonate (46.80 mg, 64.26 umol, 0.1 eq), and the resulting suspension was stirred at 85° C. for 15 hours. The reaction mixture was cooled, diluted with water (10 mL), and extracted with EtOAc (10 mL×3). The combined organic extract was washed with brine (20 mL×2), dried over Na₂SO₄, filtered, and concentrated under reduced pressure. The residue was purified by flash chromatography on SiO₂ (gradient: 0-30% ethyl acetate in petroleum ether) followed by SFC (column: DAICEL CHIRALPAK AD (250 mm*30 mm, 10 um); mobile phase: [0.1% NH₄OH in CH₃OH]; Begin B: 60; End B: 60; Gradient Time (min): 100% B Hold Time (min): Flow Rate (ml/min): 80). The pure fractions were combined and concentrated under reduced pressure to afford tert-butyl 3-[7-[8-chloro-3-(methoxymethoxy)-1-naphthyl]-2-(2,2-dimethoxyethoxy)-8-fluoro-pyrido[4,3-d]pyrimidin-4-yl]-3,8-diazabicyclo[3.2.1]octane-8-carboxylate (172 mg, 243.86 umol, 55% yield) as a yellow solid. LC/MS (ESI) m/z: 684.3 [M+H]⁺.

Step 7: Preparation of tert-butyl 3-[7-(8-chloro-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate

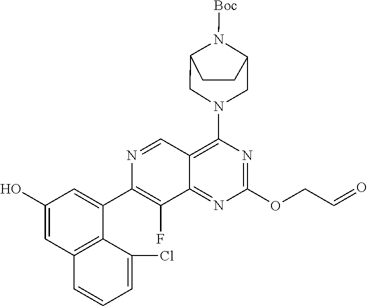 [SMILES: CC(C)(C)OC(=O)N1C2CCC1CN(c1nc(OCC=O)nc3c(F)c(-c4cc(O)cc5cccc(Cl)c45)ncc13)C2]

To tert-butyl 3-[7-[8-chloro-3-(methoxymethoxy)-1-naphthyl]-2-(2,2-dimethoxyethoxy)-8-fluoro-pyrido[4,3-d]pyrimidin-4-yl]-3,8-diazabicyclo[3.2.1]octane-8-carboxylate (172 mg, 243.86 umol, 97% purity, 1.0 eq) in acetone (429 uL) was added aqueous HCl (429 uL, 4.44 mmol, 37% purity, 18.21 eq), and the resulting suspension was stirred at 25° C. for 15 minutes. A solution of NaHCO₃ (738.72 mg, 8.79 mmol, 342 μL, 36.0 eq) in water (3 mL) was added dropwise followed by THF (3 mL) and Boc₂O (112 uL, 487.51 umol, 2.0 eq). The resulting mixture was stirred at 25° C. for 1 hour. The mixture was extracted with EtOAc (10 mL×3), and the combined organic extracts were washed with brine (20 mL), dried over Na₂SO₄, filtered, and concentrated under reduced pressure. The residue was purified by flash chromatography on SiO₂ (gradient: 0-50% Ethyl acetate in petroleum ether) to afford tert-butyl 3-[7-(8-chloro-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (120 mg, 183.83 umol, 75% yield) as a colorless solid. LC/MS (ESI) m/z: 594.1 [M+H]⁺.

Step 8: Preparation of (2S,4R)-1-[(2R)-2-(3-{[1-(2-{[7-(8-chloro-3-hydroxynaphthalen-1-yl)-4-{3,8-diazabicyclo[3.2.1]octan-3-yl}-8-fluoropyrido[4,3-d]pyrimidin-2-yl]oxy}ethyl) piperidin-4-yl]methoxy}-1,2-oxazol-5-yl)-3-methylbutanoyl]-4-hydroxy-N-(Compound 57)

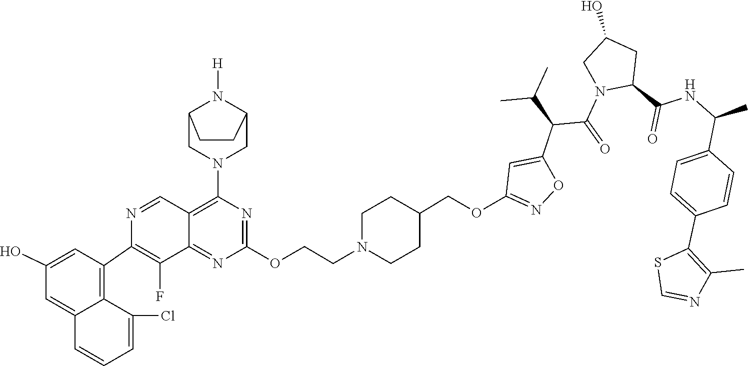 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc7cccc(Cl)c67)c(F)c5n4)CC3)no2)C(C)C)cc1]

The title compound was prepared in an analogous manner to Compound 20 starting from tert-butyl 3-[7-(8-chloro-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide, and purified by pre-HPLC (column: Phenomenex C18 75*30 mm*3 um; mobile phase: [3-43% CH₃CN in water (FA)]; Flow Rate (ml/min): 25). The pure fractions were combined and concentrated under reduced pressure, then lyophilized to afford (2S,4R)-1-[(2R)-2-(3-{[1-(2-{[7-(8-chloro-3-hydroxynaphthalen-1-yl)-4-{3,8-diazabicyclo[3.2.1]octan-3-yl}-8-fluoropyrido[4,3-d]pyrimidin-2-yl]oxy}ethyl) piperidin-4-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (52.4 mg, 48.68 umol, 41% yield) as a white solid. LC/MS (ESI) m/z: 1073.8 [M+H]⁺. ¹H NMR (400 MHz, CD₃OD) δ 9.09 (s, 1H), 8.90-8.85 (m, 1H), 8.45 (s, 1H), 7.76 (dd, J=1.6, 8.0 Hz, 1H), 7.52-7.26 (m, 7H), 7.16 (d, J=2.4 Hz, 1H), 5.99 (s, 1H), 5.07-4.98 (m, 1H), 4.83-4.73 (m, 4H), 4.51 (t, J=8.0 Hz, 1H), 4.47-4.37 (m, 1H), 4.13-4.06 (m, 2H), 4.03 (br s, 2H), 3.92-3.79 (m, 3H), 3.78-3.66 (m, 1H), 3.64-3.50 (m, 3H), 3.41-3.33 (m, 2H), 2.90-2.73 (m, 2H), 2.50-2.45 (m, 3H), 2.41-2.32 (m, 1H), 2.24-2.14 (m, 1H), 2.11-1.93 (m, 8H), 1.72-1.54 (m, 3H), 1.52 (d, J=7.2 Hz, 2H), 1.05 (d, J=6.4 Hz, 3H), 0.92-0.84 (m, 3H).

Exemplary Synthesis of (2S,4R)-1-[(2R)-2-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-(Compound 39)

Step 1: Preparation of 5-[2-(4-fluorophenyl)acetyl]-2,2-dimethyl-1,3-dioxane-4,6-dione

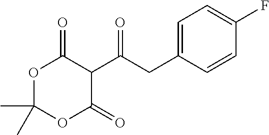 [SMILES: CC1(C)OC(=O)C(C(=O)Cc2ccc(F)cc2)C(=O)O1]

To a solution of 2-(4-fluorophenyl) acetic acid (50 g, 324.38 mmol, 1.0 eq), 2,2-dimethyl-1,3-dioxane-4,6-dione (51.4 g, 356.82 mmol, 1.1 eq), and DMAP (3.4 g, 27.57 mmol, 0.085 eq) in CH₃CN (150 mL) was added DIEA (121 mL, 697.43 mmol, 2.2 eq) followed by 2,2-dimethylpropanoyl chloride (44 mL, 356.82 mmol, 1.1 eq) slowly over 3 hours while maintaining the temperature below 45° C. The reaction mixture was stirred at 45° C. for 3 hours. The reaction was cooled to 0° C. HCl (500 mL, 1 M, 1.54 eq) was then added, and the mixture was stirred at 0° C. for 30 minutes. The mixture was filtered, and the filter cake was washed with water (CH₃CN:Water=1:4, 500 mL). The filter cake was dissolved with CH₂Cl₂ (500 mL), dried over anhydrous sodium sulfate, filtered, and concentrated in vacuum to give 5-[2-(4-fluorophenyl)acetyl]-2,2-dimethyl-1,3-dioxane-4,6-dione (84.5 g, 301.52 mmol, 93% yield) as a white solid.

Step 2: Preparation of tert-butyl 4-(4-fluorophenyl)-3-oxo-butanoate

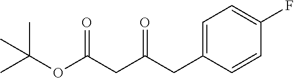 [SMILES: CC(C)(C)OC(=O)CC(=O)Cc1ccc(F)cc1]

A solution of 5-[2-(4-fluorophenyl)acetyl]-2,2-dimethyl-1,3-dioxane-4,6-dione (168 g, 601.25 mmol, 1.0 eq) in t-BuOH (500 mL) was stirred at 90° C. for 2.5 hours. The reaction mixture was concentrated in vacuum to give tert-butyl 4-(4-fluorophenyl)-3-oxo-butanoate (151 g, 598.54 mmol, 100% yield) as a yellow oil. LC/MS (ESI) m/z: 197.1 [M−56]⁺.

Step 3: Preparation of 4-(4-fluorophenyl)-3-oxo-butanoic acid

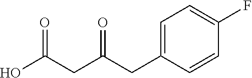 [SMILES: O=C(O)CC(=O)Cc1ccc(F)cc1]

To a solution of tert-butyl 4-(4-fluorophenyl)-3-oxo-butanoate (151 g, 598.54 mmol, 1.0 eq) in CH₂Cl₂ (300 mL) was added TFA (293 mL, 3.95 mol, 6.60 eq), and the reaction mixture was stirred at 20° C. for 1 hour. The mixture was concentrated in vacuum to give 4-(4-fluorophenyl)-3-oxo-butanoic acid (115 g, 532.28 mmol, 89% yield) as a yellow solid. LC/MS (ESI) m/z: 197.1 [M+H]⁺.

Step 4: Preparation of 7-fluoronaphthalene-1,3-diol

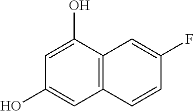 [SMILES: Oc1cc(O)c2cc(F)ccc2c1]

A solution of 4-(4-fluorophenyl)-3-oxo-butanoic acid (115 g, 586.21 mmol, 1.0 eq) in CF₃SO₃H (1200 mL) was stirred at 20° C. for 16 hours. The reaction was cooled to 0° C., and slowly poured onto ice-water (3.0 L). The resulting precipitate was filtered, and the filter cake was dissolved with ethyl acetate (200 mL×3). The combined organic phase was washed with saturated aqueous NaHCO₃ (40 mL×2) and brine (40 mL), dried over anhydrous sodium sulfate, filtered, and concentrated in vacuum to give 7-fluoronaphthalene-1,3-diol (54.4 g, 267.18 mmol, 46% yield) as a red solid. LC/MS (ESI) m/z: 179.1 [M+H]⁺. ¹H NMR (400 MHz, DMSO-d₆) δ 10.19 (s, 1H), 9.49 (s, 1H), 7.67-7.53 (m, 2H), 7.25-7.20 (m, 1H), 6.64 (s, 1H), 6.56 (s, 1H).

Step 5: Preparation of 7-fluoro-8-(2-triisopropylsilylethynyl) naphthalene-1,3-diol

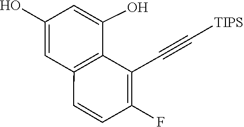

To a solution of 7-fluoronaphthalene-1,3-diol (43.5 g, 244.23 mmol, 1.0 eq), 2-bromoethynyl(triisopropyl)silane (67 g, 256.44 mmol, 1.05 eq), and KOAc (48 g, 488.45 mmol, 2.0 eq) in dioxane (300 mL) was added dichlororuthenium; 1-isopropyl-4-methyl-benzene (9.0 g, 14.65 mmol, 0.06 eq) under N₂, and the reaction mixture was stirred at 110° C. for 16 hours. The mixture was filtered and concentrated in vacuum, and the resulting residue was purified by flash chromatography on SiO₂ (gradient: 0-20% ethyl acetate in petroleum ether) to afford 7-fluoro-8-(2-triisopropylsilylethynyl) naphthalene-1,3-diol (46.3 g, 124.11 mmol, 51% yield) as a black oil. LC/MS (ESI) m/z: 359.2 [M+H]⁺. ¹H NMR (400 MHz, CDCl₃) δ 9.17 (s, 1H), 7.61-7.57 (m, 1H), 7.17 (t, J=8.8 Hz, 1H), 6.74 (d, J=2.4 Hz, 1H), 6.66 (d, J=2.0 Hz, 1H), 1.22-1.16 (m, 21H).

Step 6: Preparation of 7-fluoro-3-(methoxymethoxy)-8-(2-triisopropylsilylethynyl) naphthalen-1-ol

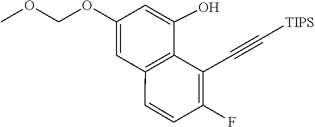

To a solution of 7-fluoro-8-(2-triisopropylsilylethynyl) naphthalene-1,3-diol (46.3 g, 129.14 mmol, 1.0 eq) in CH₂Cl₂ (450 mL) at 0° C. were added DIEA (67.5 mL, 387.43 mmol, 3.0 eq) and MOMCl (14.7 mL, 193.71 mmol, 1.5 eq), and the reaction mixture was stirred at 0° C. for 40 minutes. The reaction was quenched by addition of water (50 mL) at 0° C., then extracted with dichloromethane (50 mL×3). The combined organic extract was washed with brine (30 mL), dried over anhydrous sodium sulfate, filtered, and concentrated in vacuum. The resulting residue was purified by flash chromatography on SiO₂ (gradient: 0-15% dichloromethane in petroleum ether) to afford 7-fluoro-3-(methoxymethoxy)-8-(2-triisopropylsilylethynyl) naphthalen-1-ol (26.8 g, 62.51 mmol, 48% yield) as a yellow oil. LC/MS (ESI) m/z: 403.1 [M+H]⁺.

Step 7: Preparation of [7-fluoro-3-(methoxymethoxy)-8-(2-triisopropylsilylethynyl)-1-naphthyl]trifluoromethanesulfonate

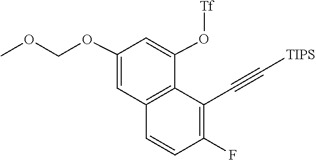

To a solution of 7-fluoro-3-(methoxymethoxy)-8-(2-triisopropylsilylethynyl) naphthalen-1-ol (21.8 g, 54.15 mmol, 1.0 eq) and DIEA (28.3 mL, 162.45 mmol, 3.0 eq) in CH₂Cl₂ (300 mL) at −40° C. was added Tf₂O (13.4 mL, 81.23 mmol, 1.5 eq), and the reaction mixture was stirred at −40° C. for 40 minutes. The reaction was quenched by addition of water (150 mL) at −40° C., warmed to 25° C., and then extracted with dichloromethane (100 mL×3). The combined organic extract was washed with brine (50 mL), dried over anhydrous sodium sulfate, and concentrated in vacuum. The resulting residue was purified by flash chromatography on SiO₂ (gradient: 0-15% dichloromethane in petroleum ether) to afford [7-fluoro-3-(methoxymethoxy)-8-(2-triisopropylsilylethynyl)-1-naphthyl]trifluoromethanesulfonate (28 g, 51.95 mmol, 96% yield) as a yellow oil.

Step 8: Preparation of 2-[2-fluoro-6-(methoxymethoxy)-8-(4,4,5,5-tetramethyl-1,3,2-dioxaborolan-2-yl)-1-naphthyl]ethynyl-triisopropyl-silane

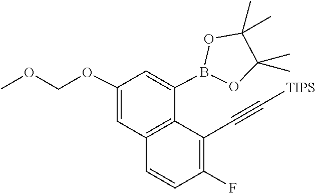

To a solution of [7-fluoro-3-(methoxymethoxy)-8-(2-triisopropylsilylethynyl)-1-naphthyl]trifluoromethanesulfonate (23.8 g, 44.52 mmol, 1.0 eq), 4,4,5,5-tetramethyl-2-(4,4,5,5-tetramethyl-1,3,2-dioxaborolan-2-yl)-1,3,2-dioxaborolane (22.6 g, 89.03 mmol, 2.0 eq), and KOAc (13.1 g, 133.55 mmol, 3.0 eq) in toluene (350 mL) was added Pd(dppf) Cl₂ (3.26 g, 4.45 mmol, 0.1 eq) under N₂, and the reaction mixture was stirred at 110° C. for 16 hours under N₂. The mixture was filtered through celite pad under vacuum and rinsed with ethyl acetate (80 mL×2). The filtrate was evaporated to dryness, and the resulting residue was purified by flash chromatography on SiO₂ (gradient: 0˜3% ethyl acetate in petroleum ether) to afford 2-[2-fluoro-6-(methoxymethoxy)-8-(4,4,5,5-tetramethyl-1,3,2-dioxaborolan-2-yl)-1-naphthyl]ethynyl-triisopropyl-silane (21.4 g, 27.97 mmol, 63% yield) as a yellow solid. LC/MS (ESI) m/z: 513.0 [M+H]⁺.

Step 9: Preparation of tert-butyl 3-[[2-(2,2-dimethoxyethoxy)-8-fluoro-7-[7-fluoro-3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate

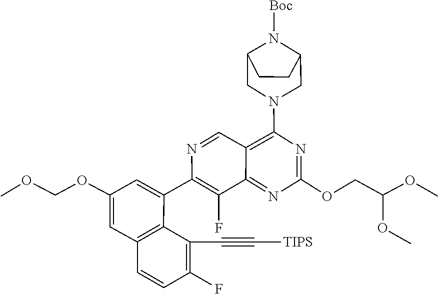

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (400 mg, 0.803 mmol, 1.0 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (494 mg, 0.964 mmol, 1.2 eq) in dioxane (10 mL) and H₂O (2 mL) were added K₃PO₄ (512 mg, 2.41 mmol, 3.0 eq) and [2-(2-aminophenyl)phenyl]palladium (1+); bis(1-adamantyl)-butyl-phosphane; methanesulfonate (117 mg, 0.161 mmol, 0.2 eq) under N₂, and the reaction mixture was stirred at 85° C. for 15 hours under N₂. The mixture was dried over anhydrous sodium sulfate, filtered, and concentrated in vacuum. The resulting residue was purified by flash chromatography on SiO₂ (gradient: 0˜27% ethyl acetate in petroleum ether) to afford tert-butyl 3-[[2-(2,2-dimethoxyethoxy)-8-fluoro-7-[7-fluoro-3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (590 mg, 0.684 mmol, 85% yield) as a yellow solid. LC/MS (ESI) m/z: 848.4 [M+H]⁺.

Step 10: Preparation of tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate

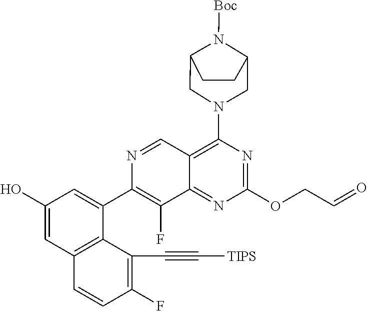

To a solution of tert-butyl 3-[[2-(2,2-dimethoxyethoxy)-8-fluoro-7-[7-fluoro-3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (630 mg, 0.743 mmol, 1.0 eq) in acetone (1.6 mL) was added aqueous HCl (1.58 mL, 12 M, 25.51 eq), and the reaction mixture was stirred at 20° C. for 15 minutes. A solution of NaHCO₃ (867 uL, 22.29 mmol, 30.0 eq) in water (3 mL), Boc₂₀ (341 uL, 1.49 mmol, 2.0 eq), and THF (3 mL) were added, and the reaction mixture was stirred at 20° C. for 1 hour. The mixture was diluted with water (20 mL), and extracted with ethyl acetate (40 mL×3). The combined organic extracts were washed with brine (20 mL), dried over anhydrous Na₂SO₄, filtered, and concentrated in vacuum. The resulting residue was purified by flash chromatography on SiO₂ (gradient: 0˜50% ethyl acetate in petroleum ether) to afford tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (420 mg, 386.23 umol, 52% yield) as a yellow solid. LC/MS (ESI) m/z: 758.4 [M+H]⁺.

Step 11: Preparation of tert-butyl 3-[8-fluoro-7-[7-fluoro-3-hydroxy-8-(2-triisopropylsilylethynyl)-1-naphthyl]-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate

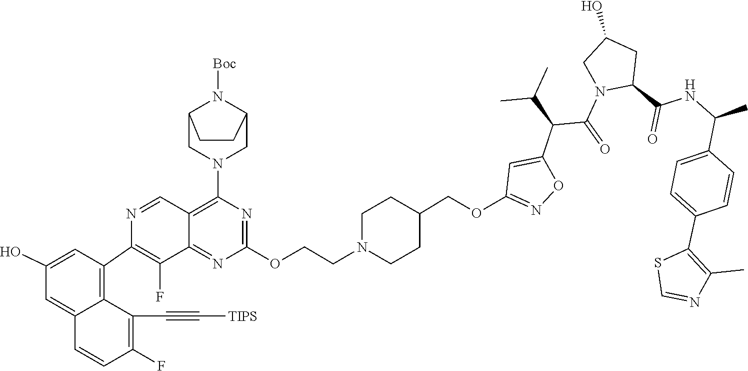

To a solution of tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (240 mg, 0.317 mmol, 1.0 eq) and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (208 mg, 0.348 mmol, 1.1 eq) in CH₂Cl₂ (10 mL) and i-PrOH (2 mL) were added acetic acid (73 uL, 1.27 mmol, 4.0 eq) and 2-methylpyridine borane (169 mg, 1.58 mmol, 5.0 eq), and the reaction mixture was stirred at 25° C. for 30 minutes. The mixture was concentrated in vacuum. The resulting residue, was dissolved with CH₂Cl₂ (5 mL) and purified by flash chromatography on SiO₂ (gradient: 0˜9% methanol in dichloromethane) to afford tert-butyl 3-[8-fluoro-7-[7-fluoro-3-hydroxy-8-(2-triisopropylsilylethynyl)-1-naphthyl]-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate (328 mg, 0.204 mmol, 54% yield) as a yellow solid. LC/MS (ESI) m/z: 669.6 [M/2+H]⁺. ¹H NMR (400 MHz, CDCl₃) δ 9.07 (d, J=16.4 Hz, 1H), 8.68 (s, 1H), 7.72-7.62 (m, 1H), 7.44-7.32 (m, 5H), 7.29 (s, 1H), 7.24-7.10 (m, 2H), 5.76 (s, 1H), 5.11-5.00 (m, 1H), 4.99-4.85 (m, 1H), 4.81-4.19 (m, 9H), 4.08-3.96 (m, 2H), 3.79-3.69 (m, 2H), 3.62-3.03 (m, 9H), 2.54-2.48 (m, 3H), 2.44-2.30 (m, 2H), 2.28-2.22 (m, 1H), 2.03-1.78 (m, 8H), 1.55-1.46 (m, 13H), 1.08-0.96 (m, 3H), 0.89-0.81 (m, 21H), 0.60-0.47 (m, 3H).

Step 12: Preparation of (2S,4R)-1-[(2R)-2-[3-[[1-[2-[4-(3,8-diazabicyclo[3.2.1]octan-3-yl)-8-fluoro-7-[7-fluoro-3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

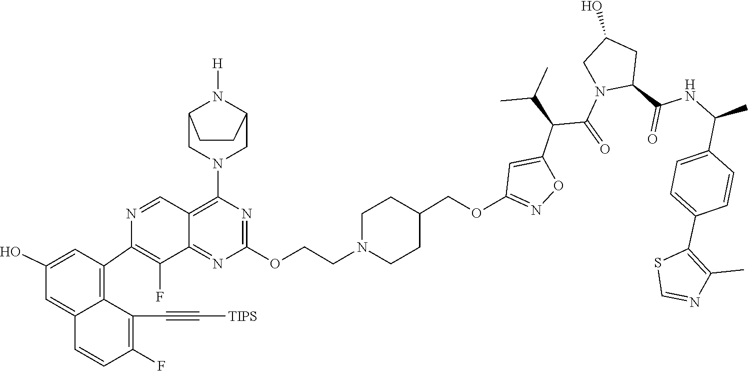

To a solution of tert-butyl 3-[8-fluoro-7-[7-fluoro-3-hydroxy-8-(2-triisopropylsilylethynyl)-1-naphthyl]-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate (328 mg, 0.245 mmol, 1.0 eq) in CH₂Cl₂ (3 mL) was added HCl/dioxane (3 mL, 4 M, 48.9 eq), and the reaction mixture was stirred at 20° C. for 1 hour. The mixture was filtered and concentrated in vacuum. The pH of the resulting residue was adjusted to pH 8˜9 by addition of saturated aqueous NaHCO₃. Dichloromethane:methanol=10:1 (30 mL) was added, and the resulting mixture was extracted with dichloromethane (30 mL×2). The combined organic extract was washed with brine (20 mL), dried over anhydrous sodium sulfate, filtered, and concentrated in vacuum to give (2S,4R)-1-[(2R)-2-[3-[[1-[2-[4-(3,8-diazabicyclo[3.2.1]octan-3-yl)-8-fluoro-7-[7-fluoro-3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (300 mg, 0.223 mmol, 91% yield) as a yellow solid. LC/MS (ESI) m/z: 619.5 [M/2+H]⁺. ¹H NMR (400 MHz, CD₃OD) δ 9.38 (s, 1H), 9.19 (s, 1H), 7.91-7.87 (m, 1H), 7.53-7.43 (m, 4H), 7.41-7.33 (m, 2H), 7.20 (s, 1H), 6.00 (s, 1H), 5.15 (d, J=13.4 Hz, 1H), 5.08-4.95 (m, 3H), 4.66-4.48 (m, 2H), 4.46-4.35 (m, 2H), 4.29-4.19 (m, 2H), 4.13 (s, 1H), 3.90-3.75 (m, 4H), 3.71-3.63 (m, 3H), 3.50-3.46 (m 1H), 3.35 (s, 1H), 3.24-3.13 (m, 2H), 2.54 (s, 3H), 2.39-2.09 (m, 9H), 1.97-1.93 (m, 1H), 1.82-1.69 (m, 2H), 1.61-1.50 (m, 3H), 1.07-1.03 (m, 3H), 0.95-0.85 (m, 21H), 0.61-0.50 (m, 3H).

Step 13: Preparation of (2S,4R)-1-[(2R)-2-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

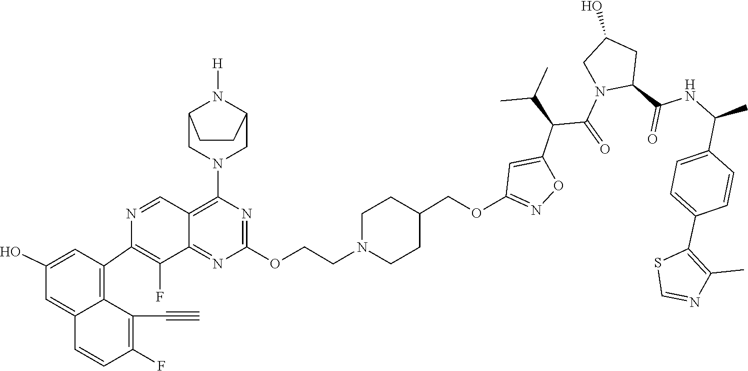 [SMILES: C#Cc1c(F)ccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of (2S,4R)-1-[(2R)-2-[3-[[1-[2-[4-(3,8-diazabicyclo[3.2.1]octan-3-yl)-8-fluoro-7-[7-fluoro-3-hydroxy-8-(2-triisopropylsilylethynyl)-1-naphthyl]pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (150 mg, 0.121 mmol, 1.0 eq) in DMF (2.0 mL) was added CsF (184 mg, 1.21 mmol, 10 eq), and the reaction mixture was stirred at 25° C. for 16 hours. The reaction mixture was filtered, and the filtrate was purified by prep-HPLC (column: Phenomenex C18 75*30 mm*3 um; mobile phase: [32-72% CH₃CN in water (NH₄HCO₃)]). The pure fractions were combined and lyophilized to afford (2S,4R)-1-[(2R)-2-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (40.5 mg, 0.0366 mmol, 30% yield) as a white solid. LC/MS (ESI) m/z: 541.8 [M/2+H]⁺. ¹H NMR (400 MHz, CD₃OD) δ 9.01 (s, 1H), 8.88 (s, 1H), 7.87-7.84 (m, 1H), 7.46-7.39 (m, 4H), 7.37-7.28 (m, 2H), 7.21 (d, J=2.4 Hz, 1H), 5.98 (s, 1H), 4.66-4.58 (m, 7H), 4.51 (t, J=8.4 Hz, 1H), 4.4-4.43 (m, 1H), 4.05 (d, J=6.0 Hz, 2H), 3.83-3.77 (m, 1H), 3.72-3.62 (m, 5H), 3.37 (s, 1H), 3.18-3.08 (m, 2H), 2.89-2.82 (m, 2H), 2.48 (s, 3H), 2.42-2.30 (m, 1H), 2.28-2.15 (m, 3H), 1.98-1.91 (m, 1H), 1.88-1.79 (m, 6H), 1.61-1.50 (m, 3H), 1.48-1.38 (m, 2H), 1.05 (d, J=6.4 Hz, 3H), 0.92-0.87 (m, 3H).

Exemplary Synthesis of [(3R,7aR)-7a-{[(4-{3,8-diazabicyclo[3.2.1]octan-3-yl}-8-fluoro-7-(3-hydroxynaphthalen-1-yl)pyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 37)

Step 1: Preparation of tert-butyl 3-[8-fluoro-7-(3-hydroxy-1-naphthyl)-2-methylsulfanyl-pyrido[4,3-d]pyrimidin-4-yl]-3,8-diazabicyclo[3.2.1]octane-8-carboxylate

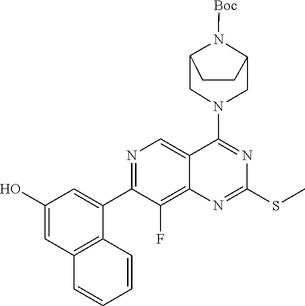 [SMILES: CSc1nc(N2CC3CCC(C2)N3C(=O)OC(C)(C)C)c2cnc(-c3cc(O)cc4ccccc34)c(F)c2n1]

To a solution of tert-butyl 3-(7-chloro-8-fluoro-2-methylsulfanyl-pyrido[4,3-d]pyrimidin-4-yl)-3,8-diazabicyclo[3.2.1]octane-8-carboxylate (300 mg, 0.682 mmol, 1.0 eq) and 4-(4,4,5,5-tetramethyl-1,3,2-dioxaborolan-2-yl) naphthalen-2-ol (368 mg, 1.36 mmol, 2.0 eq) in dioxane (6 mL) were added K₃PO₄ (1 M, 2.05 mL, 3.0 eq) and [2-(2-aminophenyl)phenyl]palladium (1+); bis(1-adamantyl)-butyl-phosphane; methanesulfonate (50 mg, 0.068 mmol, 0.1 eq) in one portion at 20° C., and the reaction mixture was stirred at 80° C. for 12 hours under N₂. The residue was dissolved in ethyl acetate, water (30 mL) was added, and the aqueous phase was extracted with ethyl acetate (40 mL×3). The combined organic extract was washed with brine (30 mL), dried with anhydrous Na₂SO₄, filtered, and concentrated in vacuum. The resulting residue was purified by flash chromatography on SiO₂ (gradient: 0˜33% ethyl acetate in petroleum ether) to afford tert-butyl 3-[8-fluoro-7-(3-hydroxy-1-naphthyl)-2-methylsulfanyl-pyrido[4,3-d]pyrimidin-4-yl]-3,8-diazabicyclo[3.2.1]octane-8-carboxylate (536 mg, 0.949 mmol, 83% yield) as a yellow solid. LC/MS (ESI) m/z: 548.2 [M+H]⁺.

Step 2: Preparation of tert-butyl 3-[8-fluoro-7-[3-(methoxymethoxy)-1-naphthyl]-2-methylsulfanyl-pyrido[4,3-d]pyrimidin-4-yl]-3,8-diazabicyclo[3.2.1]octane-8-carboxylate

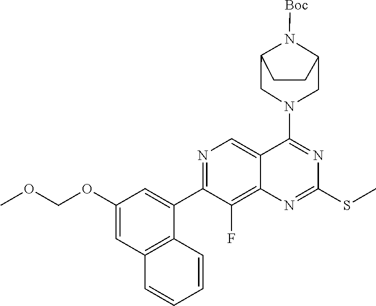

To a solution of tert-butyl 3-[8-fluoro-7-(3-hydroxy-1-naphthyl)-2-methylsulfanyl-pyrido[4,3-d]pyrimidin-4-yl]-3,8-diazabicyclo[3.2.1]octane-8-carboxylate (536 mg, 0.979 mmol, 1.0 eq) in dichloromethane (6 mL) at 0° C. were added MOMCl (118 mg, 1.47 mmol, 1.5 eq) and DIEA (2.94 mmol, 511 μL, 3.0 eq) dropwise, and the reaction mixture was stirred at 0° C. for 2 hours. The reaction was quenched by addition of water (15 mL) at 0° C., then extracted with dichloromethane (25 mL×3). The combined organic extract was washed with brine (20 mL), dried over anhydrous sodium sulfate, filtered, and concentrated in vacuum. The resulting residue was purified by flash chromatography on SiO₂ (gradient: 0˜21% ethyl acetate in petroleum ether) to afford tert-butyl 3-[8-fluoro-7-[3-(methoxymethoxy)-1-naphthyl]-2-methylsulfanyl-pyrido[4,3-d]pyrimidin-4-yl]-3,8-diazabicyclo[3.2.1]octane-8-carboxylate (390 mg, 606.39 umol, 62% yield) as a yellow solid. LC/MS (ESI) m/z: 592.1 [M+H]⁺.

Step 3: Preparation of tert-butyl 3-[8-fluoro-7-[3-(methoxymethoxy)-1-naphthyl]-2-methylsulfonyl-pyrido[4,3-d]pyrimidin-4-yl]-3,8-diazabicyclo[3.2.1]octane-8-carboxylate

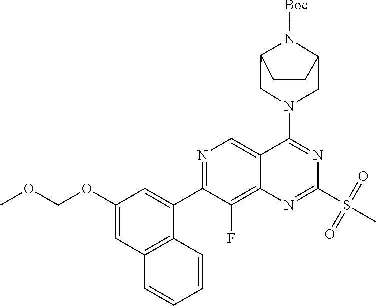

To a solution of tert-butyl 3-[8-fluoro-7-[3-(methoxymethoxy)-1-naphthyl]-2-methylsulfanyl-pyrido[4,3-d]pyrimidin-4-yl]-3,8-diazabicyclo[3.2.1]octane-8-carboxylate (340 mg, 0.575 mmol, 1.0 eq) in EtOAc (5 mL) at 0° C. was added mCPBA (350 mg, 1.72 mmol, 85% purity, 3.0 eq) in one portion, and the reaction mixture was stirred at 0° C. for 2 hours. Saturated aqueous Na₂SO₃ (10 mL) was added, and the aqueous phase was extracted with ethyl acetate (30 mL×3). The combined organic extract was washed with brine (20 mL) and saturated aqueous NaHCO₃ (20 mL×2), dried with anhydrous Na₂SO₄, filtered, and concentrated in vacuum. The resulting residue was purified by flash chromatography on SiO₂ (gradient: 0˜33% ethyl acetate in petroleum ether) to afford tert-butyl 3-[8-fluoro-7-[3-(methoxymethoxy)-1-naphthyl]-2-methylsulfonyl-pyrido[4,3-d]pyrimidin-4-yl]-3,8-diazabicyclo[3.2.1]octane-8-carboxylate (382 mg, 0.582 mmol, 88% yield) as a yellow solid. LC/MS (ESI) m/z: 624.3 [M+H]⁺.

Step 4: Preparation of tert-butyl 3-[[2-[[(3R,8R)-3-[[tert-butyl(diphenyl)silyl]oxymethyl]-1,2,3,5,6,7-hexahydropyrrolizin-8-yl]methoxy]-8-fluoro-7-[3-(methoxymethoxy)-1-naphthyl]pyrido[4,3-d]pyrimidin-4-yl]-3,8-diazabicyclo[3.2.1]octane-8-carboxylate

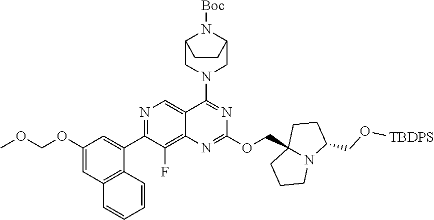

To a mixture of tert-butyl 3-[8-fluoro-7-[3-(methoxymethoxy)-1-naphthyl]-2-methylsulfonyl-pyrido[4,3-d]pyrimidin-4-yl]-3,8-diazabicyclo[3.2.1]octane-8-carboxylate (130 mg, 0.208 mmol, 1.0 eq), [(3R,8R)-3-[[tert-butyl(diphenyl)silyl]oxymethyl]-1,2,3,5,6,7-hexahydropyrrolizin-8-yl]methanol (102 mg, 0.250 mmol, 1.2 eq), and 4 Å MS (130 mg, 1.0 eq) in toluene (4 mL) at 0° C. was added t-BuONa (30 mg, 0.313 mmol, 1.5 eq) in one portion, and the reaction mixture was stirred at 0° C. for 1 hour under N₂. The pH of the reaction mixture was adjusted to pH=7 with aqueous HCl (2 M), and the resulting mixture was extracted with ethyl acetate (20 mL×3). The combined organic extract was washed with brine (20 mL), dried over anhydrous Na₂SO₄, filtered, and concentrated in vacuum. The resulting residue was purified by flash chromatography on SiO₂ (gradient: 0˜3% methanol in dichloromethane) to afford tert-butyl 3-[[2-[[(3R,8R)-3-[[tert-butyl(diphenyl)silyl]oxymethyl]-1,2,3,5,6,7-hexahydropyrrolizin-8-yl]methoxy]-8-fluoro-7-[3-(methoxymethoxy)-1-naphthyl]pyrido[4,3-d]pyrimidin-4-yl]-3,8-diazabicyclo[3.2.1]octane-8-carboxylate (225 mg, 0.177 mmol, 62% yield) as a yellow solid. LC/MS (ESI) m/z: 953.5 [M+H]⁺.

Step 5: Preparation of tert-butyl 3-[8-fluoro-2-[[(3R,8R)-3-(hydroxymethyl)-1,2,3,5,6,7-hexahydropyrrolizin-8-yl]methoxy]-7-[3-(methoxymethoxy)-1-naphthyl]pyrido[4,3-d]pyrimidin-4-yl]-3,8-diazabicyclo[3.2.1]octane-8-carboxylate

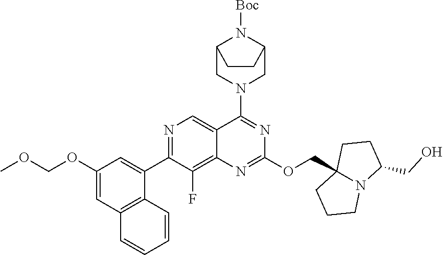 [SMILES: COCOc1cc(-c2ncc3c(N4CC5CCC(C4)N5C(=O)OC(C)(C)C)nc(OC[C@]45CCCN4[C@@H](CO)CC5)nc3c2F)c2ccccc2c1]

To a solution of tert-butyl 3-[[2-[[(3R,8R)-3-[[tert-butyl(diphenyl)silyl]oxymethyl]-1,2,3,5,6,7-hexahydropyrrolizin-8-yl]methoxy]-8-fluoro-7-[3-(methoxymethoxy)-1-naphthyl]pyrido[4,3-d]pyrimidin-4-yl]-3,8-diazabicyclo[3.2.1]octane-8-carboxylate (225 mg, 0.236 mmol, 1.0 eq) in DMF (4 mL) at 30° C. was added CsF (287 mg, 1.89 mmol, 8.0 eq), and the reaction mixture was stirred at 30° C. for 12 hours. Water (20 mL) was added, and the aqueous phase was extracted with ethyl acetate (30 mL×3). The combined organic extract was washed with brine (20 mL), dried over anhydrous Na₂SO₄, filtered, and concentrated in vacuum. The resulting residue was purified by flash chromatography on SiO₂ (gradient: 0˜10% (3N NH₃ in methanol) in dichloromethane) to afford tert-butyl 3-[8-fluoro-2-[[(3R,8R)-3-(hydroxymethyl)-1,2,3,5,6,7-hexahydropyrrolizin-8-yl]methoxy]-7-[3-(methoxymethoxy)-1-naphthyl]pyrido[4,3-d]pyrimidin-4-yl]-3,8-diazabicyclo[3.2.1]octane-8-carboxylate (132 mg, 0.159 mmol, 67% yield) as a white solid. LC/MS (ESI) m/z: 715.4 [M+H]⁺.

Step 6: Preparation of tert-butyl 3-[8-fluoro-2-[[(3R,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]-7-[3-(methoxymethoxy)-1-naphthyl]pyrido[4,3-d]pyrimidin-4-yl]-3,8-diazabicyclo[3.2.1]octane-8-carboxylate

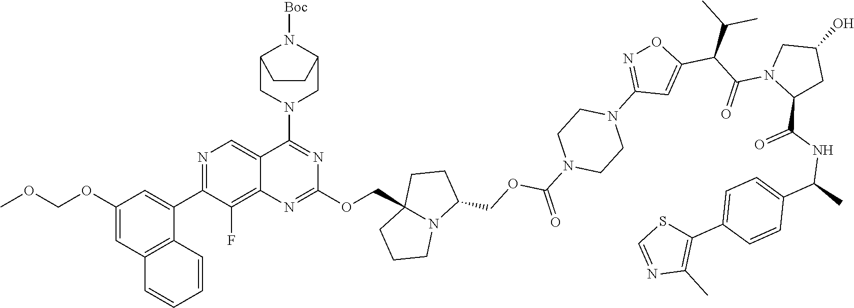 [SMILES: COCOc1cc(-c2ncc3c(N4CC5CCC(C4)N5C(=O)OC(C)(C)C)nc(OC[C@]45CCCN4[C@@H](COC(=O)N4CCN(c6cc([C@H](C(=O)N7C[C@H](O)C[C@H]7C(=O)N[C@@H](C)c7ccc(-c8scnc8C)cc7)C(C)C)on6)CC4)CC5)nc3c2F)c2ccccc2c1]

To tert-butyl 3-[8-fluoro-2-[[(3R,8R)-3-(hydroxymethyl)-1,2,3,5,6,7-hexahydropyrrolizin-8-yl]methoxy]-7-[3-(methoxymethoxy)-1-naphthyl]pyrido[4,3-d]pyrimidin-4-yl]-3,8-diazabicyclo[3.2.1]octane-8-carboxylate (132 mg, 0.185 mmol, 1.0 eq) in tetrahydrofuran (4 mL) were added TEA (1.48 mmol, 206 μL, 8.0 eq) and (2S,4R)-4-hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (126 mg, 0.222 mmol, 1.2 eq), and the resulting suspension was stirred at 20° C. for 12 hours. (4-Nitrophenyl) carbonochloridate (74 mg, 0.370 mmol, 2.0 eq) was added, and the reaction mixture was stirred at 28° C. for 24 hours. Additional TEA (0.554 mmol, 77 μL, 3.0 eq) and (4-nitrophenyl) carbonochloridate (74 mg, 0.369 mmol, 2.0 eq) were added, and the resulting suspension was stirred at 28° C. for 1 hour. The reaction mixture was filtered, and the filtrate concentrated under reduced pressure. The resulting residue was purified by flash chromatography on SiO₂ (gradient: 0˜6% methanol in dichloromethane) to afford tert-butyl 3-[8-fluoro-2-[[(3R,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]-7-[3-(methoxymethoxy)-1-naphthyl]pyrido[4,3-d]pyrimidin-4-yl]-3,8-diazabicyclo[3.2.1]octane-8-carboxylate (248 mg, 0.152 mmol, 82% yield) as a white solid. LC/MS (ESI) m/z: 1307.6 [M+H]⁺.

Step 7: Preparation of [(3R,7aR)-7a-{[(4-{3,8-diazabicyclo[3.2.1]octan-3-yl}-8-fluoro-7-(3-hydroxynaphthalen-1-yl)pyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

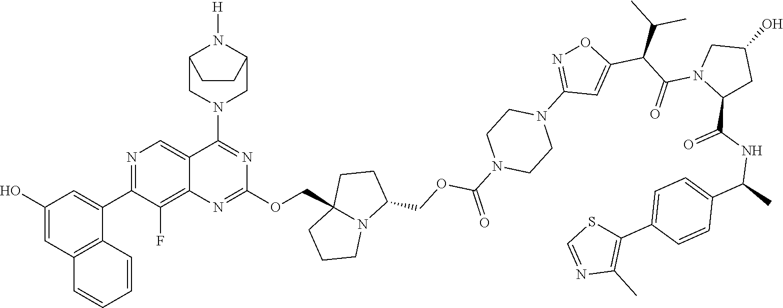 [SMILES: Cc1ncsc1-c1ccc([C@H](C)NC(=O)[C@@H]2C[C@@H](O)CN2C(=O)[C@@H](c2cc(N3CCN(C(=O)OC[C@H]4CC[C@@]5(COc6nc(N7CC8CCC(C7)N8)c7cnc(-c8cc(O)cc9ccccc89)c(F)c7n6)CCCN45)CC3)no2)C(C)C)cc1]

To tert-butyl 3-[8-fluoro-2-[[(3R,8R)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]-7-[3-(methoxymethoxy)-1-naphthyl]pyrido[4,3-d]pyrimidin-4-yl]-3,8-diazabicyclo[3.2.1]octane-8-carboxylate (248 mg, 0.152 mmol, 80% purity, 1.0 eq) in dichloromethane (4 mL) at 20° C. was added HCl/dioxane (4 M, 3.20 mL, 84.4 eq) in one portion, and the reaction mixture was stirred at 20° C. for 1 hour. Petroleum ether (20 mL) was added, and the mixture was filtered through celite pad under vacuum. The cake was dissolved in dichloromethane (10 mL) and mixture was poured onto a solution of NaHCO₃. The resulting mixture was extracted with dichloromethane/methanol (120 mL, 10:1, V/V). The combined organic extract was washed with brine (30 mL), dried with anhydrous Na₂SO₄, filtered, and concentrated in vacuum. The resulting residue was purified by prep-HPLC (column: Phenomenex C18 75*30 mm*3 um; mobile phase: [0-40% CH₃CN in water (FA)]). The pure fractions were combined, then lyophilized to afford [(3R,7aR)-7a-{[(4-{3,8-diazabicyclo[3.2.1]octan-3-yl}-8-fluoro-7-(3-hydroxynaphthalen-1-yl)pyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (107.7 mg, 0.088 mmol, 58% yield, FA) as a white solid. LC/MS (ESI) m/z: 1763.9 [M+H]⁺. ¹H NMR (400 MHz, CD₃OD) δ 9.22-9.08 (m, 1H), 8.88 (s, 1H), 8.46 (d, J=4.0 Hz, 1H), 7.76 (d, J=8.0 Hz, 1H), 7.57-7.49 (m, 1H), 7.47-7.32 (m, 5H), 7.31-7.17 (m, 3H), 6.16-6.02 (m, 1H), 5.10-4.97 (m, 1H), 4.77-4.68 (m, 3H), 4.54-4.48 (m, 2H), 4.62-4.48 (m, 1H), 4.45-4.34 (m, 2H), 4.19 (d, J=8.4 Hz, 1H), 3.95-3.77 (m, 5H), 3.66-3.46 (m, 7H), 3.24-3.14 (m, 4H), 2.54-2.44 (m, 3H), 2.44-2.29 (m, 3H), 2.27-1.80 (m, 13H), 1.59-1.48 (m, 3H), 1.05 (d, J=6.5 Hz, 3H), 0.93-0.81 (m, 3H).

Exemplary Synthesis of (2S,4R)-1-[(2R)-2-[3-(7-{2-[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 26)

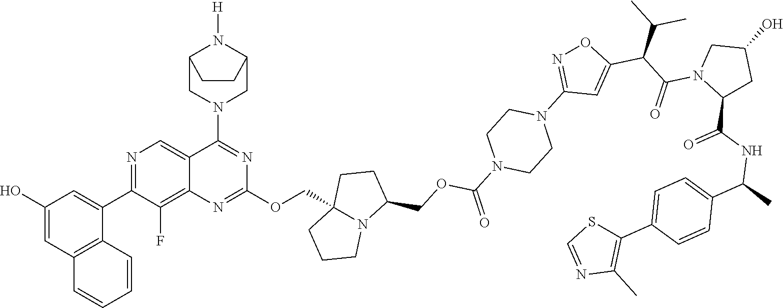 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@]5(COc6nc(N7CC8CCC(C7)N8)c7cnc(-c8cc(O)cc9ccccc89)c(F)c7n6)CCCN45)CC3)no2)C(C)C)cc1]

The title compound was prepared in an analogous manner to Compound 37 starting from tert-butyl 3-[8-fluoro-7-[3-(methoxymethoxy)-1-naphthyl]-2-methylsulfonyl-pyrido[4,3-d]pyrimidin-4-yl]-3,8-diazabicyclo[3.2.1]octane-8-carboxylate and [(3S,8S)-3-[[tert-butyl(diphenyl)silyl]oxymethyl]-1,2,3,5,6,7-hexahydropyrrolizin-8-yl]methanol, and purified by prep-HPLC (column: Phenomenex C18 75*30 mm*3 um; mobile phase: [2-42% CH₃CN in water (FA)]). The pure fractions were combined, then lyophilized to afford (2S,4R)-1-[(2R)-2-[3-(7-{2-[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (70.8 mg, 0.057 mmol, 42% yield, FA) as a white solid. LC/MS (ESI) m/z: 1163.8 [M+H]⁺. ¹H NMR (400 MHz, CD₃OD) δ 9.22-9.13 (m, 1H), 8.90-8.82 (m, 1H), 8.42 (s, 1H), 7.76 (d, J=8.0 Hz, 1H), 7.59-7.50 (m, 1H), 7.48-7.33 (m, 5H), 7.31-7.18 (m, 3H), 6.16-6.02 (m, 1H), 5.11-4.96 (m, 1H), 4.78-4.69 (m, 3H), 4.60 (d, J=12.2 Hz, 1H), 4.57-4.47 (m, 2H), 4.46-4.34 (m, 2H), 4.27-4.10 (m, 1H), 3.92 (s, 2H), 3.84 (dd, J=4.0, 11.2 Hz, 3H), 3.67-3.46 (m, 7H), 3.25-3.12 (m, 4H), 2.52-2.31 (m, 6H), 2.30-1.82 (m, 13H), 1.62-1.48 (m, 3H), 1.05 (d, J=6.4 Hz, 3H), 0.94-0.83 (m, 3H).

Exemplary Synthesis of (2S,4R)-1-[(2R)-2-[3-(7-{2-[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 36)

Step 1: Preparation of tert-butyl 3-[8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]-7-[3-hydroxy-8-(2-triisopropylsilylethynyl)-1-naphthyl]pyrido[4,3-d]pyrimidin-4-yl]-3,8-diazabicyclo[3.2.1]octane-8-carboxylate

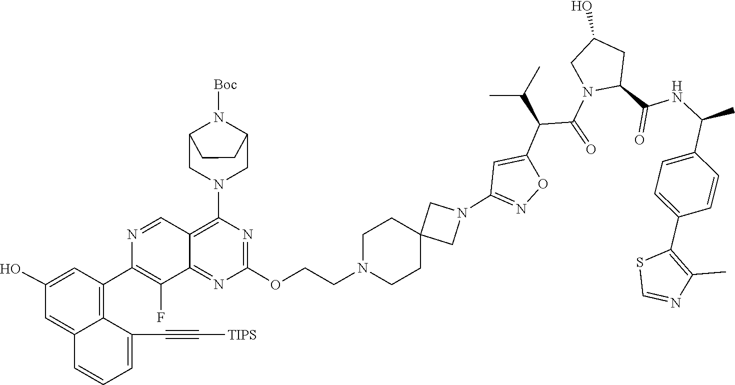

To a solution of tert-butyl 3-[8-fluoro-7-[3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (200 mg, 0.27 mmol, 1.0 eq) and (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (164.0 mg, 0.270 mmol, 1.0 eq) in CH₂Cl₂ (3 mL) and i-PrOH (3 mL) were added acetic acid (1.35 mmol, 77.0 uL, 5.0 eq) and 2-methylpyridine borane (145.0 mg, 1.35 mmol, 5.0 eq), and the reaction mixture was stirred at 25° C. for 1 hour. The pH of the mixture was adjusted to pH ˜8 by addition of triethylamine. The resulting mixture was concentrated, and the residue was purified by flash chromatography on SiO₂ (gradient: 0˜7% methanol in dichloromethane) to afford tert-butyl 3-[8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]-7-[3-hydroxy-8-(2-triisopropylsilylethynyl)-1-naphthyl]pyrido[4,3-d]pyrimidin-4-yl]-3,8-diazabicyclo[3.2.1]octane-8-carboxylate (230 mg, 0.173 mmol, 64% yield) as a yellow solid. LC/MS (ESI) m/z: 1330.5 [M+H]⁺.

Step 2: Preparation of tert-butyl 3-[7-(8-ethynyl-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate

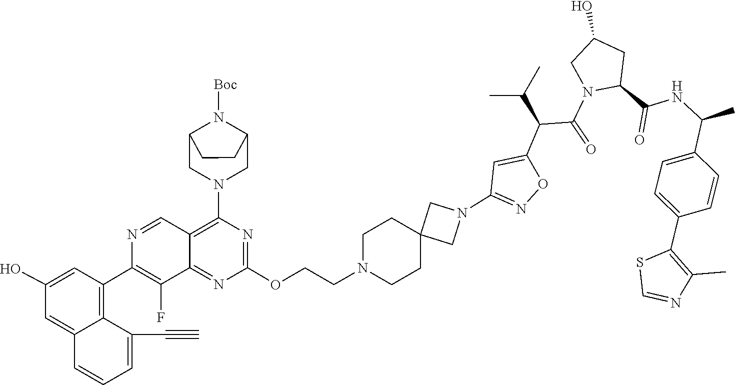 [SMILES: C#Cc1cccc2cc(O)cc(-c3ncc4c(N5CC6CCC(C5)N6C(=O)OC(C)(C)C)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12]

To a solution of tert-butyl 3-[8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]-7-[3-hydroxy-8-(2-triisopropylsilylethynyl)-1-naphthyl]pyrido[4,3-d]pyrimidin-4-yl]-3,8-diazabicyclo[3.2.1]octane-8-carboxylate (200 mg, 0.150 mmol, 1.0 eq) in DMF (3 mL) was added CsF (115.0 mg, 0.751 mmol, 5.0 eq), and the reaction mixture was stirred at 25° C. for 10 hours. The reaction mixture was diluted with water (15 mL) and filtered. The solid was washed with CH₂Cl₂ (15 mL×3), and the filtrate was concentrated to afford tert-butyl 3-[7-(8-ethynyl-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (170 mg, crude). LC/MS (ESI) m/z: 1174.3 [M+H]⁺.

Step 3: Preparation of (2S,4R)-1-[(2R)-2-[3-(7-{2-[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide

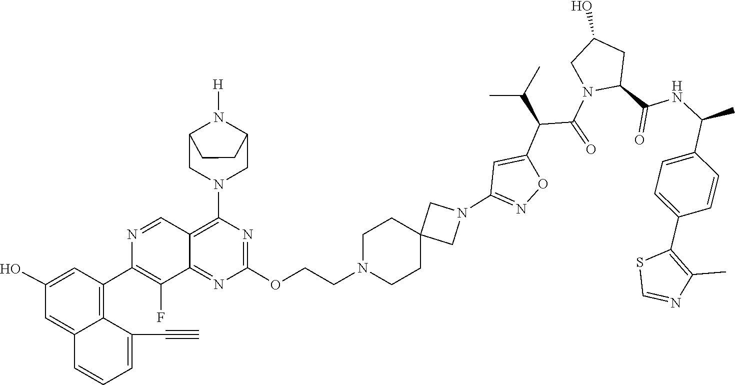 [SMILES: C#Cc1cccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12]

A solution of tert-butyl 3-[7-(8-ethynyl-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (120 mg, 0.102 mmol, 1.0 eq) in HCOOH (10 mL) was stirred at 25° C. for 1.5 hours. The mixture was concentrated, diluted with CH₂Cl₂ (5 mL), and basified with saturated aqueous NaHCO₃ until pH ˜8. The resulting mixture was concentrated under reduced pressure to afford the aqueous phase, then lyophilized to afford crude product as a yellow solid. The crude product was purified by preparative HPLC (column: DAICEL CHIRALPAK IC (250 mm*30 mm, 10 um); mobile phase: [45% CH₃CN in EtOH (0.1% NH₄OH)]). The pure fractions were combined, concentrated, and lyophilized to afford (2S,4R)-1-[(2R)-2-[3-(7-{2-[(4-{3,8-diazabicyclo[3.2.1]octan-3-yl}-7-(8-ethynyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (58.5 mg, 0.054 mmol, 52% yield) as a yellow solid. LC/MS (ESI) m/z: 1074.5 [M+H]⁺. ¹H NMR (400 MHz, CD₃OD) δ 9.00 (d, J=1.8 Hz, 1H), 8.88 (s, 1H), 7.81 (d, J=8.4 Hz, 1H), 7.55-7.28 (m, 7H), 7.22-7.10 (m, 1H), 5.90-5.76 (m, 1H), 5.07-4.98 (m, 1H), 4.75-4.35 (m, 7H), 3.87-3.54 (m, 11H), 3.04 (s, 1H), 2.85 (t, J=5.2 Hz, 2H), 2.60 (s, 3H), 2.50-2.45 (m, 3H), 2.41-2.29 (m, 1H), 2.22-2.10 (m, 1H), 2.00-1.73 (m, 9H), 1.58-1.45 (m, 3H), 1.04 (d, J=6.4 Hz, 3H), 0.94-0.85 (m, 3H).

Exemplary Synthesis of [(3R,7aR)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 34)

Step 1: Preparation of tert-butyl 3-[7-(8-ethyl-1-naphthyl)-8-fluoro-2-methylsulfanyl-pyrido[4,3-d]pyrimidin-4-yl]-3,8-diazabicyclo[3.2.1]octane-8-carboxylate

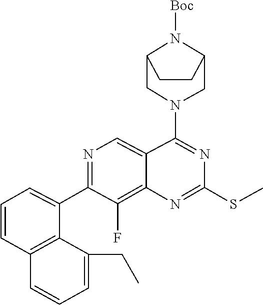 [SMILES: CCc1cccc2cccc(-c3ncc4c(N5CC6CCC(C5)N6C(=O)OC(C)(C)C)nc(SC)nc4c3F)c12]

To tert-butyl 3-(7-chloro-8-fluoro-2-methylsulfanyl-pyrido[4,3-d]pyrimidin-4-yl)-3,8-diazabicyclo[3.2.1]octane-8-carboxylate (550 mg, 1.25 mmol, 1.0 eq) in dioxane (9.6 mL) and water (2.4 mL) were added 2-(8-ethyl-1-naphthyl)-4,4,5,5-tetramethyl-1,3,2-dioxaborolane (529.17 mg, 1.88 mmol, 1.5 eq), K₃PO₄ (530.7 mg, 2.50 mmol, 2.0 eq), and [2-(2-aminophenyl)phenyl]palladium (1+); bis(1-adamantyl)-butyl-phosphane; methanesulfonate (182.1 mg, 250.04 umol, 0.2 eq) under N₂ atmosphere, and the reaction mixture was stirred at 100° C. for 15 hours. The reaction was cooled and diluted with water (10 mL). The resulting mixture was extracted with EtOAc (20 mL×3), and the combined organic extract was washed with brine (20 mL*2), dried over Na₂SO₄, filtered, and concentrated under reduced pressure. The resulting residue was purified by flash chromatography on SiO₂ (gradient: 0-20% ethyl acetate in petroleum ether) to afford tert-butyl 3-[7-(8-ethyl-1-naphthyl)-8-fluoro-2-methylsulfanyl-pyrido[4,3-d]pyrimidin-4-yl]-3,8-diazabicyclo[3.2.1]octane-8-carboxylate (667 mg, 1.02 mmol, 82% yield) as a yellow solid. LC/MS (ESI) m/z: 560.3 [M+H]⁺.

Step 2: Preparation of tert-butyl 3-[7-(8-ethyl-1-naphthyl)-8-fluoro-2-methylsulfonyl-pyrido[4,3-d]pyrimidin-4-yl]-3,8-diazabicyclo[3.2.1]octane-8-carboxylate

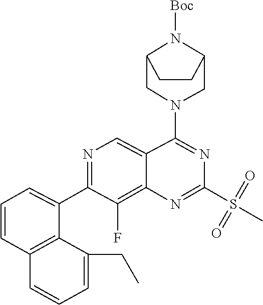 [SMILES: CCc1cccc2cccc(-c3ncc4c(N5CC6CCC(C5)N6C(=O)OC(C)(C)C)nc(S(C)(=O)=O)nc4c3F)c12]

To tert-butyl 3-[7-(8-ethyl-1-naphthyl)-8-fluoro-2-methylsulfanyl-pyrido[4,3-d]pyrimidin-4-yl]-3,8-diazabicyclo[3.2.1]octane-8-carboxylate (667 mg, 1.02 mmol, 86% purity, 1.0 eq) in EtOAc (14 mL) at 0° C. was added mCPBA (624.21 mg, 3.07 mmol, 85% purity, 3.0 eq), and the reaction mixture was stirred at 0° C. for 1.5 hours. The reaction was quenched with saturated aqueous Na₂S₂O₃ (5 mL), and the resulting mixture was stirred at 20° C. for 1 hour. The organic layer was separated and washed with saturated aqueous NaHCO₃ (20 mL×3), brine (20 mL×3), dried over Na₂SO₄, filtered, and concentrated under reduced pressure. The resulting residue was purified by flash chromatography on SiO₂ (gradient: 0-40% ethyl acetate in petroleum ether) to afford tert-butyl 3-[7-(8-ethyl-1-naphthyl)-8-fluoro-2-methylsulfonyl-pyrido[4,3-d]pyrimidin-4-yl]-3,8-diazabicyclo[3.2.1]octane-8-carboxylate (440 mg, 713.88 umol, 70% yield) as a yellow solid. LC/MS (ESI) m/z: 592.2 [M+H]⁺.

Step 3: Preparation of tert-butyl 3-[[2-[[(3R,8R)-3-[[tert-butyl(diphenyl)silyl]oxymethyl]-1,2,3,5,6,7-hexahydropyrrolizin-8-yl]methoxy]-7-(8-ethyl-1-naphthyl)-8-fluoro-pyrido[4,3-d]pyrimidin-4-yl]-3,8-diazabicyclo[3.2.1]octane-8-carboxylate

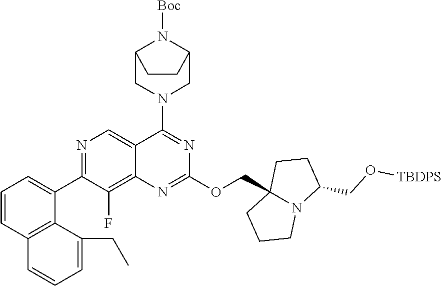

To a mixture of tert-butyl 3-[7-(8-ethyl-1-naphthyl)-8-fluoro-2-methylsulfonyl-pyrido[4,3-d]pyrimidin-4-yl]-3,8-diazabicyclo[3.2.1]octane-8-carboxylate (190 mg, 308.27 umol, 96% purity, 1.0 eq), [(3R,8R)-3-[[tert-butyl(diphenyl)silyl]oxymethyl]-1,2,3,5,6,7-hexahydropyrrolizin-8-yl]methanol (174.18 mg, 369.92 umol, 87% purity, 1.2 eq), and 4 Å MS (200 mg, 1.0 eq) in toluene (4 mL) at 0° C. was added t-BuONa (44.4 mg, 462.40 umol, 1.5 eq), and the reaction mixture was stirred at 0° C. for 30 minutes. The reaction was quenched with aqueous HCl (2 M) to pH ˜5 and extracted with EtOAc (20 mL×2). The organic extracts were combined, dried over Na₂SO₄, filtered, and concentrated under reduced pressure. The residue was purified by flash chromatography on SiO₂ (gradient: 0-8% CH₃OH in CH₂Cl₂) to afford tert-butyl 3-[[2-[[(3R,8R)-3-[[tert-butyl(diphenyl)silyl]oxymethyl]-1,2,3,5,6,7-hexahydropyrrolizin-8-yl]methoxy]-7-(8-ethyl-1-naphthyl)-8-fluoro-pyrido[4,3-d]pyrimidin-4-yl]-3,8-diazabicyclo[3.2.1]octane-8-carboxylate (261 mg, 220.14 umol, 71% yield) as a yellow solid. LC/MS (ESI) m/z: 921.4 [M+H]⁺.

Step 4: Preparation of tert-butyl 3-[7-(8-ethyl-1-naphthyl)-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

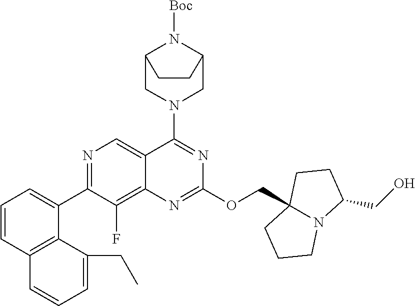 [SMILES: CCc1cccc2cccc(-c3ncc4c(N5CC6CCC(C5)N6C(=O)OC(C)(C)C)nc(OC[C@]56CCCN5[C@@H](CO)CC6)nc4c3F)c12]

To tert-butyl 3-[[2-[[(3R,8R)-3-[[tert-butyl(diphenyl)silyl]oxymethyl]-1,2,3,5,6,7-hexahydropyrrolizin-8-yl]methoxy]-7-(8-ethyl-1-naphthyl)-8-fluoro-pyrido[4,3-d]pyrimidin-4-yl]-3,8-diazabicyclo[3.2.1]octane-8-carboxylate (260 mg, 219.30 umol, 77.7% purity, 1.0 eq) in DMF (5 mL) was added CsF (266.49 mg, 1.75 mmol, 8.0 eq), and the reaction mixture was stirred at 30° C. for 7 hours. The mixture was diluted with water (5 mL) and extracted with EtOAc (10 mL×3). The combined organic extract was washed with brine (20 mL), dried over Na₂SO₄, filtered, and concentrated under reduced pressure. The residue was purified by flash chromatography on SiO₂ (gradient: 0-10% (3.5 M NH₃ in CH₃OH) in CH₂Cl₂) to afford tert-butyl 3-[7-(8-ethyl-1-naphthyl)-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (109 mg, 138.88 umol, 63% yield) as a yellow solid. LC/MS (ESI) m/z: 683.2 [M+H]⁺.

Step 5: Preparation of tert-butyl 3-[7-(8-ethyl-1-naphthyl)-8-fluoro-2-[[(3R,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

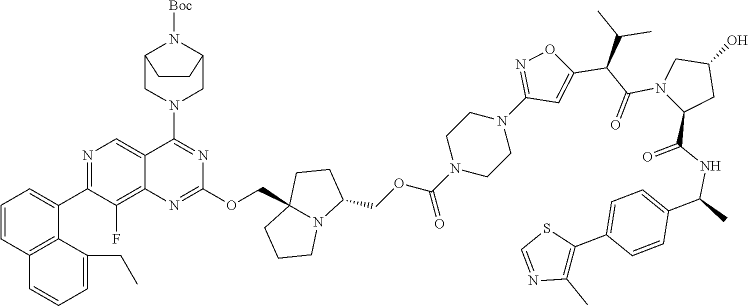 [SMILES: CCc1cccc2cccc(-c3ncc4c(N5CC6CCC(C5)N6C(=O)OC(C)(C)C)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To tert-butyl 3-[7-(8-ethyl-1-naphthyl)-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (109 mg, 138.88 umol, 87% purity, 1.0 eq) in THF (4 mL) were added TEA (155 uL, 1.11 mmol, 8.0 eq) and (4-nitrophenyl) carbonochloridate (56.0 mg, 277.76 umol, 2.0 eq), and the reaction mixture was stirred at 20° C. for 15 hours. (4-Nitrophenyl) carbonochloridate (8.40 mg, 41.66 umol, 0.3 eq) was added, and the reaction mixture was stirred at 30° C. for 20 hours. (2S,4R)-4-Hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (94.5 mg, 166.65 umol, 1.2 eq) and TEA (58 uL, 416.64 umol, 3.0 eq) were added, and the reaction mixture was stirred at 30° C. for 1 hour. The reaction mixture was filtered, and the filtrate was concentrated under reduced pressure. The residue was purified by flash chromatography on SiO₂ (gradient: 0-10% CH₃OH in CH₂Cl₂) to afford tert-butyl 3-[7-(8-ethyl-1-naphthyl)-8-fluoro-2-[[(3R,8R)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (160 mg, 95.33 umol, 69% yield) as a yellow solid. LC/MS (ESI) m/z: 1275.4 [M+H]⁺.

Step 6: Preparation of [(3R,7aR)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

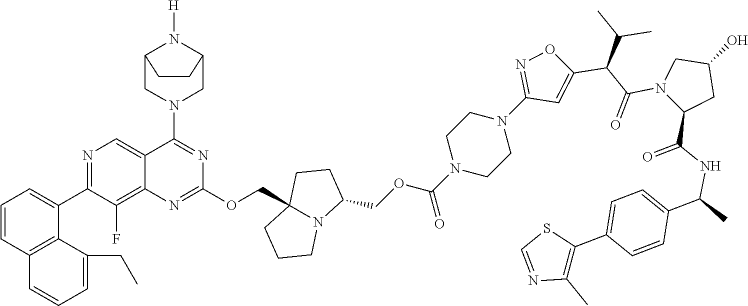 [SMILES: CCc1cccc2cccc(-c3ncc4c(N5CC6CCC(C5)N6)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

A mixture of tert-butyl 3-[7-(8-ethyl-1-naphthyl)-8-fluoro-2-[[(3R,8R)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (160 mg, 95.33 umol, 76% purity, 1.0 eq) in HCOOH (9 mL) was stirred at 20° C. for 2 hours. The mixture was concentrated under reduced pressure, and the crude product was purified by pre-HPLC (column: YMC Triart 30*150 mm*7 um; mobile phase: [16-56% CH₃CN in water (FA)]). The pure fractions were combined and concentrated under reduced pressure, and were then lyophilized to afford [(3R,7aR)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (56.8 mg, 48.27 umol, 51% yield, FA salt) as a white solid. LC/MS (ESI) m/z: 1076.0 [M+H]⁺. ¹H NMR (400 MHz, CD₃OD) δ 9.13 (s, 1H), 8.88 (s, 1H), 8.09-8.02 (m, 1H), 7.87 (br d, J=8.0 Hz, 1H), 7.60-7.54 (m, 1H), 7.52-7.47 (m, 1H), 7.46-7.32 (m, 6H), 6.16-6.07 (m, 1H), 5.07-5.00 (m, 1H), 4.81-4.67 (m, 4H), 4.61-4.48 (m, 3H), 4.46-4.35 (m, 2H), 4.18 (br d, J=1.6 Hz, 1H), 3.97-3.77 (m, 5H), 3.67-3.47 (m, 7H), 3.23 (br s, 4H), 2.50-2.45 (m, 3H), 2.45-2.30 (m, 5H), 2.23-1.89 (m, 12H), 1.60-1.47 (m, 3H), 1.05 (br d, J=6.4 Hz, 3H), 0.95-0.84 (m, 6H).

Exemplary Synthesis of [(3S,7aS)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 35)

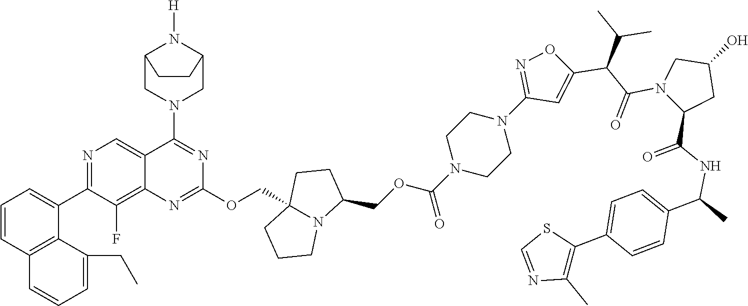 [SMILES: CCc1cccc2cccc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 34 starting from [(3R,8R)-8-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate, and purified by pre-HPLC (column: Phenomenex C18 75*30 mm*3 um; mobile phase: [6-46% CH₃CN in water (FA)]). The pure fractions were combined and concentrated under reduced pressure, then lyophilized to afford [(3S,7aS)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (103.2 mg, 87.73 umol, 57% yield, FA salt) as a white solid. LC/MS (ESI) m/z: 1076.0 [M+H]⁺. ¹H NMR (400 MHz, CD₃OD) δ 9.16-9.10 (m, 1H), 8.88 (s, 1H), 8.05 (d, J=8.4 Hz, 1H), 7.87 (d, J=8.0 Hz, 1H), 7.65-7.30 (m, 8H), 6.16-6.06 (m, 1H), 5.03 (q, J=6.8 Hz, 1H), 4.82-4.68 (m, 4H), 4.62-4.47 (m, 3H), 4.47-4.35 (m, 2H), 4.27-4.15 (m, 1H), 4.03-3.78 (m, 5H), 3.68-3.48 (m, 7H), 3.23 (br s, 4H), 2.50-2.45 (m, 3H), 2.45-2.29 (m, 5H), 2.23-1.88 (m, 12H), 1.60-1.48 (m, 3H), 1.05 (br d, J=6.4 Hz, 3H), 0.97-0.84 (m, 6H).

Exemplary Synthesis of (2S,4R)-1-[(2R)-2-[3-(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 33)

Step 1: Preparation of tert-butyl 3-[8-fluoro-7-[7-fluoro-3-hydroxy-8-(2-triisopropylsilylethynyl)-1-naphthyl]-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate

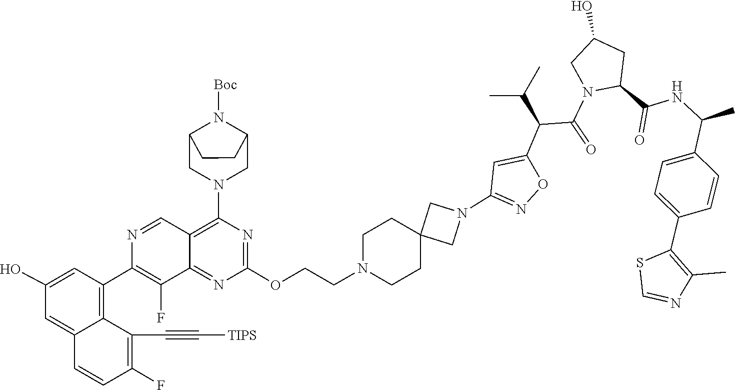

To a solution of tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (420 mg, 0.386 mmol, 69.7% purity, 1.0 eq) and (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (0.234 mg, 0.386 mmol, 1.0 eq) in CH₂Cl₂ (4 mL) and i-PrOH (4 mL) were added acetic acid (0.088 mL, 1.54 mmol, d=1.05 g/mL, 4.0 eq) and 2-methylpyridine borane (124 mg, 1.16 mmol, 3.0 eq), and the reaction mixture was stirred at 20° C. for 2 hours. The mixture was concentrated, and the resulting residue was purified by flash chromatography on SiO₂ (gradient: 0˜5% methanol in dichloromethane) to afford tert-butyl 3-[8-fluoro-7-[7-fluoro-3-hydroxy-8-(2-triisopropylsilylethynyl)-1-naphthyl]-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (460 mg, 0.317 mmol, 82% yield) as a yellow solid. LC/MS (ESI) m/z: 1349.7 [M+H]⁺.

Step 2: Preparation of tert-butyl 3-[7-(8-ethynyl-7-fluoro-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate

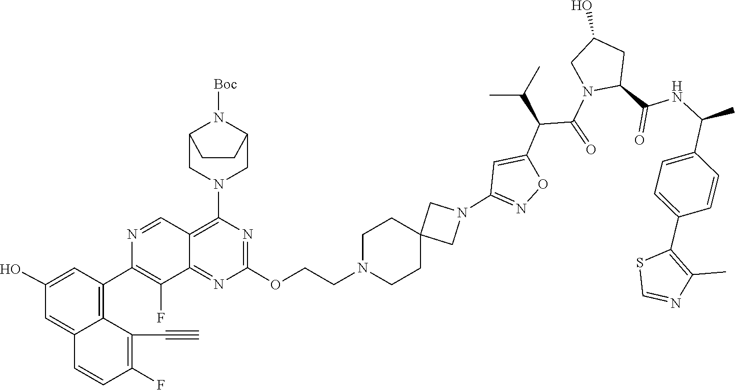 [SMILES: C#Cc1c(F)ccc2cc(O)cc(-c3ncc4c(N5CC6CCC(C5)N6C(=O)OC(C)(C)C)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12]

To a solution of tert-butyl 3-[8-fluoro-7-[7-fluoro-3-hydroxy-8-(2-triisopropylsilylethynyl)-1-naphthyl]-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (160 mg, 0.109 mmol, 92% purity, 1.0 eq) in DMF (1.5 mL) was added CsF (83 mg, 0.546 mmol, 5.0 eq), and the reaction mixture was stirred at 20° C. for 12 hours. The reaction was quenched by addition of H₂O (5 mL) and filtered. The resulting filter cake was washed with dichloromethane (15 mL×3), and the combined organic extract was dried over anhydrous Na₂SO₄, filtered, and concentrated to afford tert-butyl 3-[7-(8-ethynyl-7-fluoro-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (136 mg, crude) as a yellow solid. LC/MS (ESI) m/z: 1192.4 [M+H]⁺.

Step 3: Preparation of (2S,4R)-1-[(2R)-2-[3-(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide

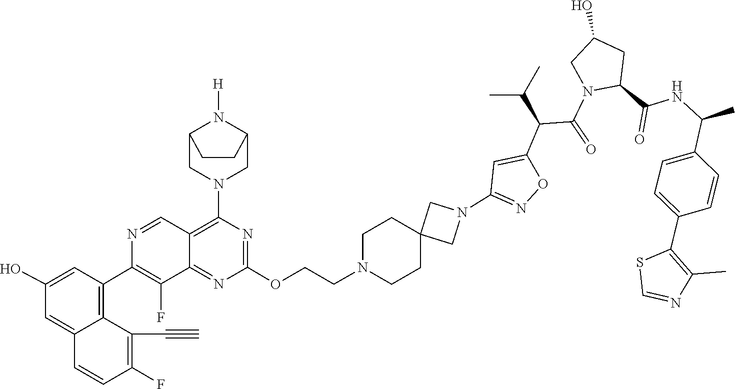 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12]

A solution of tert-butyl 3-[7-(8-ethynyl-7-fluoro-3-hydroxy-1-naphthyl)-8-fluoro-2-[2-[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (136 mg, 0.114 mmol, 1.0 eq) in HCOOH (10 mL) was stirred at 20° C. for 1.5 hours. The mixture was concentrated over vacuum, adjusted to pH=8˜9 by portion wise addition of saturated aqueous NaHCO₃, and lyophilized to afford the crude product as a yellow solid. The crude product was purified by HPLC (Condition: [50% CH₃CN in EtOH (0.1% NH₄OH)]; Gradient time: 10 min; column: ChiralPak IH, 250*30 mm; Flow Rate: 80 mL/min). The pure fractions were combined and concentrated under reduced pressure. The product was diluted with water (15 mL) and CH₃CN (15 mL), then extracted with n-hexane (20 mL×5). The aqueous phase was lyophilized to afford (2S,4R)-1-[(2R)-2-[3-(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (83.8 mg, 0.075 mmol, 66% yield) as a white solid. LC/MS (ESI) m/z: 1092.8 [M+H]⁺. ¹H NMR (400 MHz, CD₃OD) δ 9.01 (d, J=1.6 Hz, 1H), 8.88 (s, 1H), 7.86 (dd, J=5.6, 9.2 Hz, 1H), 7.46-7.29 (m, 6H), 7.21 (d, J=2.0 Hz, 1H), 5.87-5.81 (m, 1H), 5.08-4.96 (m, 1H), 4.66-4.55 (m, 4H), 4.50 (t, J=8.0 Hz, 1H), 4.43 (d, J=2.0 Hz, 1H), 3.83 (dd, J=4.0, 10.8 Hz, 1H), 3.79-3.70 (m, 2H), 3.70-3.54 (m, 9H), 3.39-3.36 (m, 1H), 2.84 (t, J=5.6 Hz, 2H), 2.66-2.52 (m, 3H), 2.49-2.47 (m, 3H), 2.39-2.31 (m, 1H), 2.23-2.14 (m, 1H), 1.98-1.91 (m, 1H), 1.90-1.76 (m, 8H), 1.55-1.46 (m, 3H), 1.04 (d, J=6.4 Hz, 3H), 0.93-0.86 (m, 3H).

Exemplary Synthesis of (2S,4R)-1-[(2R)-2-[3-({1-[2-({7-[3-chloro-5-hydroxy-2-(trifluoromethyl)phenyl]-4-{3,8-diazabicyclo[3.2.1]octan-3-yl}-8-fluoropyrido[4,3-d]pyrimidin-2-yl}oxy)ethyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 24)

Step 1:2-[3-bromo-5-chloro-4-(trifluoromethyl)phenyl]-4,4,5,5-tetramethyl-1,3,2-dioxaborolane

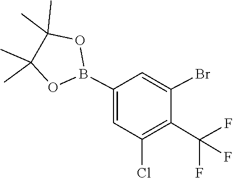 [SMILES: CC1(C)OB(c2cc(Cl)c(C(F)(F)F)c(Br)c2)OC1(C)C]

To a solution of 1-bromo-3-chloro-2-(trifluoromethyl)benzene (6.5 g, 25.05 mmol, 1.0 eq) and 4,4,5,5-tetramethyl-1,3,2-dioxaborolane (6.4 g, 50.11 mmol, 7.3 mL, 2.0 eq) in THF (100 mL) were added dtbpy (807 mg, 3.01 mmol, 0.12 eq) and (1Z,5Z)-cycloocta-1,5-diene; 2,4-dimethyl-BLAHbicyclo[1.1.0]butane (1.66 g, 2.51 mmol, 0.1 eq), and the reaction mixture was stirred at 60° C. for 3 hours under N₂. The mixture was concentrated to afford 2-[3-bromo-5-chloro-4-(trifluoromethyl)phenyl]-4,4,5,5-tetramethyl-1,3,2-dioxaborolane (9.6 g, crude) as a black brown oil.

Step 2: Preparation of 3-bromo-5-chloro-4-(trifluoromethyl) phenol

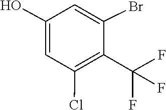 [SMILES: Oc1cc(Cl)c(C(F)(F)F)c(Br)c1]

To a solution of 2-[3-bromo-5-chloro-4-(trifluoromethyl)phenyl]-4,4,5,5-tetramethyl-1,3,2-dioxaborolane (9.6 g, 24.91 mmol, 1.0 eq) in THF (100 mL) and H₂O (50 mL) were added acetic acid (44.8 g, 747.25 mmol, 42.7 mL, 30.0 eq) and H₂O₂ (56.5 g, 498.17 mmol, 47.8 mL, 30% purity, 20 eq), and the reaction mixture was stirred at 10° C. for 1 hour. Water (100 mL) and EtOAc (200 mL) were added, and the organic layer was separated, washed with saturated Na₂S₂O₃ followed by brine (200 mL), dried over anhydrous sodium sulfate, filtered, and concentrated. The resulting residue was purified by flash chromatography on SiO₂ (gradient: 0˜10% ethyl acetate in petroleum ether) to afford 3-bromo-5-chloro-4-(trifluoromethyl) phenol (2.4 g, 8.71 mmol, 35% yield) as a yellow oil. ¹H NMR (400 MHz, DMSO-d₆) δ 11.24 (s, 1H), 7.21 (d, J=2.4 Hz, 1H), 7.03 (d, J=2.4 Hz, 1H).

Step 3: Preparation of 1-bromo-3-chloro-5-(methoxymethoxy)-2-(trifluoromethyl)benzene

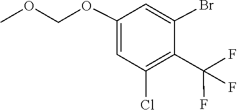 [SMILES: COCOc1cc(Cl)c(C(F)(F)F)c(Br)c1]

To a solution of 3-bromo-5-chloro-4-(trifluoromethyl) phenol (2.4 g, 8.71 mmol, 1.0 eq) and DIEA (26.14 mmol, 4.5 mL, 3.0 eq) in CH₂Cl₂ (30 mL) at 0° C. was added MOMCl (13.07 mmol, 993 μL, 1.5 eq) dropwise, and the reaction mixture was stirred at 20° C. for 1 hour. The reaction was quenched with water (20 mL), and the organic layer was dried over Na₂SO₄ and concentrated. The resulting residue was purified by flash chromatography on SiO₂ (gradient: 0˜6% ethyl acetate) to afford 1-bromo-3-chloro-5-(methoxymethoxy)-2-(trifluoromethyl)benzene (1.7 g, 5.32 mmol, 61% yield) as a light yellow oil. ¹H NMR (400 MHz, CDCl₃) δ 7.34 (s, 1H), 7.15 (s, 1H), 5.20 (s, 2H), 3.48 (s, 3H).

Step 4: Preparation of 2-[3-chloro-5-(methoxymethoxy)-2-(trifluoromethyl)phenyl]-4,4,5,5-tetramethyl-1,3,2-dioxaborolane

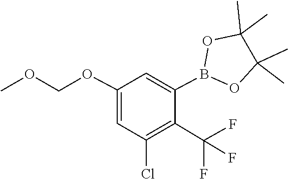

To a solution of 1-bromo-3-chloro-5-(methoxymethoxy)-2-(trifluoromethyl)benzene (1.7 g, 5.32 mmol, 1.0 eq) and 4,4,5,5-tetramethyl-2-(4,4,5,5-tetramethyl-1,3,2-dioxaborolan-2-yl)-1,3,2-dioxaborolane (2.7 g, 10.64 mmol, 2.0 eq) in dioxane (30 mL) were added KOAc (1.3 g, 13.30 mmol, 2.5 eq) and Pd(dppf) Cl₂ (389 mg, 532.08 umol, 0.1 eq), and the reaction mixture was stirred at 100° C. for 15 hours under N₂. The mixture was diluted with ethyl acetate (90 mL) and washed with water (50 mL×2). The organic extract was washed with brine (50 mL), dried over Na₂SO₄, filtered, and concentrated under reduced pressure. The resulting residue was purified by preparative HPLC (Eluent: 50˜95% CH₃CN in water (0.225% FA)). The pure fractions were combined and lyophilized under reduced pressure to afford product 2-[3-chloro-5-(methoxymethoxy)-2-(trifluoromethyl)phenyl]-4,4,5,5-tetramethyl-1,3,2-dioxaborolane (1.1 g, 3.00 mmol, 56% yield, FA salt) as a gray solid. ¹H NMR (400 MHz, CDCl₃) δ 7.11 (s, 1H), 6.90 (s, 1H), 5.12 (s, 2H), 3.37 (s, 3H), 1.29 (s, 12H).

Step 5: Preparation of tert-butyl 3-[7-[3-chloro-5-(methoxymethoxy)-2-(trifluoromethyl)phenyl]-2-(2,2-dimethoxyethoxy)-8-fluoro-pyrido[4,3-d]pyrimidin-4-yl]-3,8-diazabicyclo[3.2.1]octane-8-carboxylate

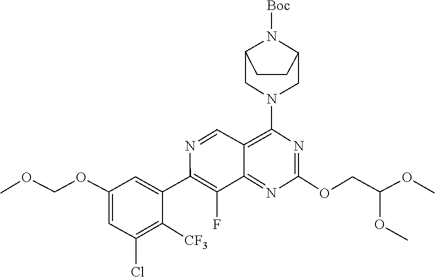

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (400 mg, 803.30 umol, 1.0 eq) and 2-[3-chloro-5-(methoxymethoxy)-2-(trifluoromethyl)phenyl]-4,4,5,5-tetramethyl-1,3,2-dioxaborolane (442 mg, 1.20 mmol, 1.5 eq) in H₂O (1 mL) and THF (10 mL) were added DavePhos Pd G3 (61 mg, 80.33 umol, 0.1 eq) and Cs₂CO₃ (785 mg, 2.41 mmol, 3.0 eq), and the reaction mixture was stirred at 90° C. for 15 hours under N₂. The mixture was concentrated, and the resulting residue was purified by flash chromatography on SiO₂ (gradient: 0˜28% THF in petroleum ether) to afford tert-butyl 3-[7-[3-chloro-5-(methoxymethoxy)-2-(trifluoromethyl)phenyl]-2-(2,2-dimethoxyethoxy)-8-fluoro-pyrido[4,3-d]pyrimidin-4-yl]-3,8-diazabicyclo[3.2.1]octane-8-carboxylate (310 mg, 397.38 umol, 49% yield) as a yellow oil. LC/MS (ESI) m/z: 703.2. [M+H]⁺.

Step 6: Preparation of tert-butyl 3-[7-[3-chloro-5-hydroxy-2-(trifluoromethyl)phenyl]-8-fluoro-2-(2-oxoethoxy)pyrido[4,3-d]pyrimidin-4-yl]-3,8-diazabicyclo[3.2.1]octane-8-carboxylate

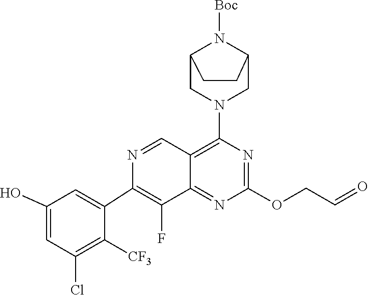 [SMILES: CC(C)(C)OC(=O)N1C2CCC1CN(c1nc(OCC=O)nc3c(F)c(-c4cc(O)cc(Cl)c4C(F)(F)F)ncc13)C2]

To a solution of tert-butyl 3-[7-[3-chloro-5-(methoxymethoxy)-2-(trifluoromethyl)phenyl]-2-(2,2-dimethoxyethoxy)-8-fluoro-pyrido[4,3-d]pyrimidin-4-yl]-3,8-diazabicyclo[3.2.1]octane-8-carboxylate (310 mg, 441.54 umol, 1.0 eq) in acetone (1 mL) was added aqueous HCl (12 M, 736 uL), and the reaction mixture was stirred at 15° C. for 10 minutes. The mixture was poured onto a solution of NaHCO₃ (1.11 g, 13.25 mmol) in H₂O (5 mL) and THF (5 mL). Boc₂O (241 mg, 1.10 mmol, 2.5 eq) was then added, and the reaction mixture was stirred at 15° C. for 1 hours. The mixture was extracted with ethyl acetate (30 mL×2), and the combined organic extract was washed with brine (30 mL), dried over Na₂SO₄, filtered, and concentrated under reduced pressure. The resulting residue was purified by flash chromatography on SiO₂ (gradient: 0˜35% THF in petroleum ether) to afford tert-butyl 3-[7-[3-chloro-5-hydroxy-2-(trifluoromethyl)phenyl]-8-fluoro-2-(2-oxoethoxy)pyrido[4,3-d]pyrimidin-4-yl]-3,8-diazabicyclo[3.2.1]octane-8-carboxylate (120 mg, 145.10 umol, 33% yield) as a light yellow solid. LC/MS (ESI) m/z: 630.2. [M+H₂O]⁺.

Step 7: Preparation of tert-butyl 3-[7-[3-chloro-5-hydroxy-2-(trifluoromethyl)phenyl]-8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate

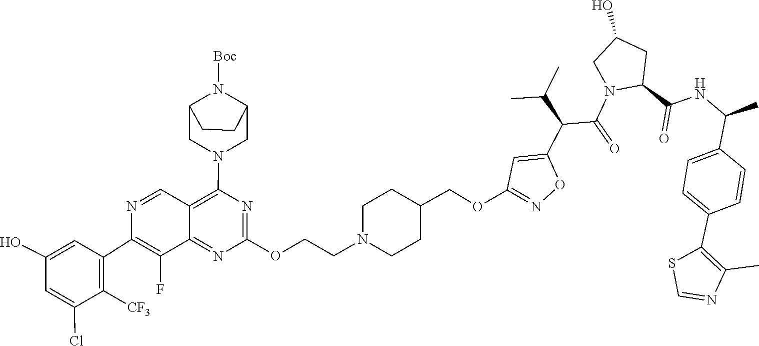 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6C(=O)OC(C)(C)C)c5cnc(-c6cc(O)cc(Cl)c6C(F)(F)F)c(F)c5n4)CC3)no2)C(C)C)cc1]

To a solution of tert-butyl 3-[7-[3-chloro-5-hydroxy-2-(trifluoromethyl)phenyl]-8-fluoro-2-(2-oxoethoxy)pyrido[4,3-d]pyrimidin-4-yl]-3,8-diazabicyclo[3.2.1]octane-8-carboxylate (120 mg, 196.09 umol, 1.0 eq) and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (117 mg, 196.09 umol, 1.0 eq) in CH₂Cl₂ (1.5 mL) and i-PrOH (1.5 mL) were added acetic acid (980.44 umol, 56 μL, 5 eq) and 2-methylpyridine borane (105 mg, 980.44 umol, 5.0 eq), and the reaction mixture was stirred at 15° C. for 15 hours. The mixture was concentrated, and the resulting residue was purified by prep-TLC (SiO₂, CH₂Cl₂:CH₃OH=10:1) to give the product tert-butyl 3-[7-[3-chloro-5-hydroxy-2-(trifluoromethyl)phenyl]-8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate (200 mg, 120.83 umol, 62% yield) as a light yellow solid. LC/MS (ESI) m/z: 1191.2. [M+H]⁺.

Step 8: Preparation of (2S,4R)-1-[(2R)-2-[3-({1-[2-({7-[3-chloro-5-hydroxy-2-(trifluoromethyl)phenyl]-4-{3,8-diazabicyclo[3.2.1]octan-3-yl}-8-fluoropyrido[4,3-d]pyrimidin-2-yl}oxy)ethyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide

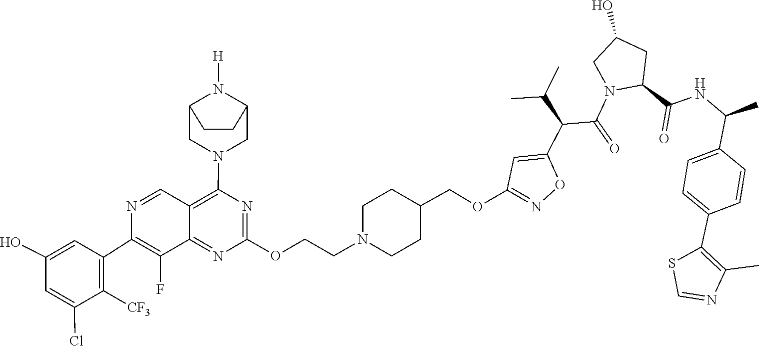 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc(Cl)c6C(F)(F)F)c(F)c5n4)CC3)no2)C(C)C)cc1]

A solution of tert-butyl 3-[7-[3-chloro-5-hydroxy-2-(trifluoromethyl)phenyl]-8-fluoro-2-[2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]ethoxy]pyrido[4,3-d]pyrimidin-4-yl]-3,8-diazabicyclo[3.2.1]octane-8-carboxylate (200 mg, 167.82 umol, 1.0 eq) in HCOOH (3 mL) was stirred at 15° C. for 1.5 hours. The mixture was concentrated, and the resulting residue was purified by preparative HPLC (Eluent: 6˜46% CH₃CN in water (0.225% FA)). The pure fractions were combined and lyophilized under reduced pressure to afford (2S,4R)-1-[(2R)-2-[3-({1-[2-({7-[3-chloro-5-hydroxy-2-(trifluoromethyl)phenyl]-4-{3,8-diazabicyclo[3.2.1]octan-3-yl}-8-fluoropyrido[4,3-d]pyrimidin-2-yl}oxy)ethyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (62 mg, 56.17 umol, 33% yield, FA salt) as a white solid. LC/MS (ESI) m/z: 1091.8. [M+H]⁺. ¹H NMR (400 MHz, CD₃OD) δ 9.08 (s, 1H), 8.88 (s, 1H), 8.43 (s, 1H), 7.46-7.37 (m, 4H), 7.15 (s, 1H), 6.76 (s, 1H), 6.00 (s, 1H), 5.06-4.97 (m, 1H), 4.79-4.71 (m, 5H), 4.50 (t, J=8.0 Hz, 1H), 4.44-4.40 (m, 1H), 4.10 (d, J=6.0 Hz, 2H), 4.00 (s, 2H), 3.85-3.82 (m, 3H), 3.68 (d, J=10.0 Hz, 1H), 3.62 (d, J=10.4 Hz, 1H), 3.54-3.48 (m, 2H), 3.37-3.34 (m, 1H), 2.82 (t, J=12.0 Hz, 2H), 2.48 (s, 3H), 2.41-2.31 (m, 1H), 2.21-2.16 (m, 1H), 2.06-1.92 (m, 8H), 1.68-1.51 (m, 5H), 1.05 (d, J=6.8 Hz, 3H), 0.92-0.88 (m, 3H).

Exemplary Synthesis of [(2S,5S)-5-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-1-methylpyrrolidin-2-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 50) Step: 1 Preparation of (2R)-1-benzyloxyhex-5-en-2-ol

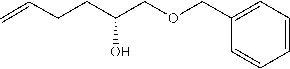

To (2R)-2-(benzyloxymethyl) oxirane (5.0 g, 30.45 mmol, 1.0 eq) in THF (50 mL) 0° C. was added CuI (580 mg, 3.05 mmol, 0.1 eq) followed by allyl(chloro)magnesium (2.0 M, 60.90 mL, 4.0 eq) dropwise, and the reaction mixture was stirred at 0° C. for 1 hour. The reaction was quenched with aqueous saturated NH₄Cl (20 mL) and extracted with EtOAc (3×20 mL). The combined organic extracts were dried over Na₂SO₄, filtered, and concentrated under reduced pressure. The resulting residue was purified by flash chromatography on SiO₂ (gradient: 0-5% EtOAc in petroleum ether) to afford (2R)-1-benzyloxyhex-5-en-2-ol (5.43 g, 18.69 mmol, 61% yield) as a colorless oil. LC/MS (ESI) m/z: 207.0 [M+H]⁺.

Step 2: Preparation of 2-[[(1S)-1-(benzyloxymethyl) pent-4-enyl]isoindoline-1,3-dione

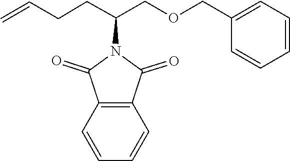 [SMILES: C=CCC[C@@H](COCc1ccccc1)N1C(=O)c2ccccc2C1=O]

To a mixture of (2R)-1-benzyloxyhex-5-en-2-ol (7.53 g, 25.92 mmol, 71% purity, 1.0 eq), isoindoline-1,3-dione (4.58 g, 31.12 mmol, 1.2 eq), and PPh₃ (8.16 g, 31.10 mmol, 1.2 eq) in THF at 0° C. (80 mL) was added DIAD (6.0 mL, 31.10 mmol, 1.2 eq), and the reaction mixture was stirred at 30° C. for 15 hours. The mixture was concentrated under reduced pressure, and the resulting residue was dissolved in EtOAc (50 mL). The organic mixture was washed with water (20 mL), brine (2×20 mL), dried over Na₂SO₄, filtered, and concentrated under reduced pressure. The resulting residue was purified by flash chromatography on SiO₂ (gradient: 0-10% EtOAc in petroleum ether) to afford 2-[[(1S)-1-(benzyloxymethyl) pent-4-enyl]isoindoline-1,3-dione (9.71 g, 17.37 mmol, 67% yield) as a colorless oil. LC/MS (ESI) m/z: 336.0 [M+H]⁺.

Step 3: Preparation of (2S)-1-benzyloxyhex-5-en-2-amine

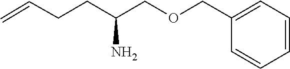

To 2-[[(1S)-1-(benzyloxymethyl) pent-4-enyl]isoindoline-1,3-dione (9.71 g, 17.37 mmol, 60% purity, 1.0 eq) in EtOH (100 mL) was added N₂H₄—H₂O (1.69 mL, 34.74 mmol, 2.0 eq), and the resulting suspension was stirred at 80° C. for 15 hours. The mixture was filtered, and the filtrate was concentrated under reduced pressure. The resulting residue was poured onto aq. NaHCO₃ (50 mL) and extracted with CH₂Cl₂ (3×50 mL). The organic extracts was dried over anhydrous Na₂SO₄, then concentrated under reduced pressure to afford (2S)-1-benzyloxyhex-5-en-2-amine (5.5 g, crude) as a colorless oil. LC/MS (ESI) m/z: 206.1 [M+H]⁺.

Step 4: Preparation of N-[(1S)-1-(benzyloxymethyl) pent-4-enyl]benzamide

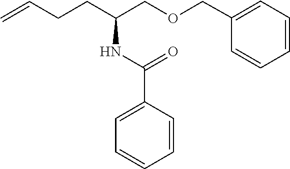 [SMILES: C=CCC[C@@H](COCc1ccccc1)NC(=O)c1ccccc1]

To (2S)-1-benzyloxyhex-5-en-2-amine (5.5 g, 26.79 mmol, 1.0 eq) and triethylamine (11.2 mL, 80.37 mmol, 3.0 eq) in CH₂Cl₂ (55 mL) at 0° C. was added benzoyl chloride (3.1 mL, 26.79 mmol, 1.0 eq) dropwise, and the reaction mixture was stirred at 20° C. for 15 hours. The organic mixture was washed with water (2×30 mL), dried over Na₂SO₄, filtered, and concentrated under reduced pressure. The resulting residue was purified by flash chromatography on SiO₂ (gradient: 0-10% EtOAc in petroleum ether) to afford N-[(1S)-1-(benzyloxymethyl) pent-4-enyl]benzamide (5.9 g, 17.92 mmol, 67% yield) as a white solid. LC/MS (ESI) m/z: 310.1 [M+H]⁺.

Step 5: Preparation of [(2S,5S)-5-(benzyloxymethyl) pyrrolidin-2-yl]methyl benzoate

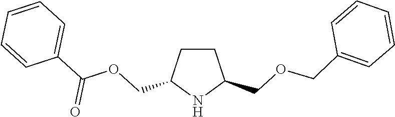 [SMILES: O=C(OC[C@@H]1CC[C@@H](COCc2ccccc2)N1)c1ccccc1]

To N-[(1S)-1-(benzyloxymethyl) pent-4-enyl]benzamide (500 mg, 1.52 mmol, 94% purity, 1.0 eq) in CH₃CN (15 mL) and water (5 mL) was added I₂ (1.16 g, 4.56 mmol, 3.0 eq), and the reaction mixture was stirred at 20° C. for 15 hours. The reaction was quenched with saturated aqueous Na₂SO₃ (10 mL), then extracted with EtOAc (3×20 mL). The combined organic extract was washed with brine (2×50 mL), dried over Na₂SO₄, filtered, and concentrated under reduced pressure to afford [(2S,5S)-5-(benzyloxymethyl) pyrrolidin-2-yl]methyl benzoate (731 mg, 1.03 mmol, crude) as a white solid. LC/MS (ESI) m/z: 326.0 [M+H]⁺.

Step 6: Preparation of tert-butyl(2S,5S)-2-(benzoyloxymethyl)-5-(benzyloxymethyl) pyrrolidine-1-carboxylate

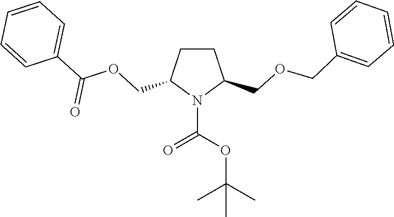 [SMILES: CC(C)(C)OC(=O)N1[C@H](COCc2ccccc2)CC[C@H]1COC(=O)c1ccccc1]

To [(2S,5S)-5-(benzyloxymethyl) pyrrolidin-2-yl]methyl benzoate (5.34 g, 16.41 mmol, 1.0 eq) in CH₂Cl₂ (50 mL) was added triethylamine (6.85 mL, 49.23 mmol, 3.0 eq) followed by Boc₂O (7.54 mL, 32.82 mmol, 2.0 eq), and the reaction mixture was stirred at 20° C. for 2 hours. The mixture was washed with water (2×30 mL), brine (2×30 mL), dried over Na₂SO₄, filtered, and concentrated under reduced pressure. The resulting residue was purified by flash chromatography on SiO₂ (gradient 0-10% EtOAc in petroleum ether). Impure fractions were combined and concentrated under reduced pressure to afford tert-butyl(2S,5S)-2-(benzoyloxymethyl)-5-(benzyloxymethyl) pyrrolidine-1-carboxylate (3.44 g, 7.68 mmol, 47% yield, 95% purity) as a colorless oil. The pure fractions were combined and concentrated under reduced pressure to afford tert-butyl(2S,5S)-2-(benzoyloxymethyl)-5-(benzyloxymethyl) pyrrolidine-1-carboxylate (1.6 g, 3.68 mmol, 22% yield, 98% purity) as a colorless oil. LC/MS (ESI) m/z: 426.1 [M+H]⁺. ¹H NMR (400 MHz, CD₃OD) δ 8.08-7.99 (m, 2H), 7.62-7.53 (m, 1H), 7.45 (dt, J=4.0, 7.6 Hz, 2H), 7.39-7.28 (m, 5H), 4.62-4.37 (m, 4H), 4.31-4.18 (m, 1H), 4.15-3.32 (m, 4H), 2.27-2.06 (m, 2H), 2.05-1.97 (m, 1H), 1.96-1.83 (m, 1H), 1.50 (s, 4H), 1.42 (s, 4H).

Step 7: Preparation of [(2S,5S)-5-(benzyloxymethyl) pyrrolidin-2-yl]methyl benzoate

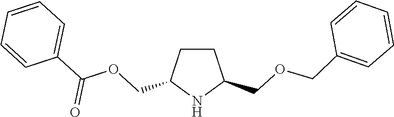 [SMILES: O=C(OC[C@@H]1CC[C@@H](COCc2ccccc2)N1)c1ccccc1]

To a solution of tert-butyl(2S,5S)-2-(benzoyloxymethyl)-5-(benzyloxymethyl) pyrrolidine-1-carboxylate (3.6 g, 8.46 mmol, 1.0 eq) in CH₂Cl₂ (54 mL) was added TFA (18.0 mL, 243.11 mmol, 28.74 eq), and the reaction mixture was stirred at 20° C. for 1 hour. The reaction mixture was concentrated under reduced pressure, and the resulting residue was dissolved in water (25 mL) adjusting to pH ˜8 by addition of with saturated aqueous NaHCO₃. The aqueous mixture was extracted with CH₂Cl₂ (3×50 mL), and the combined organic layer was dried over Na₂SO₄, filtered, and concentrated under reduced pressure to afford [(2S,5S)-5-(benzyloxymethyl) pyrrolidin-2-yl]methyl benzoate (2.75 g, crude) as a white solid. LC/MS (ESI) m/z: 326.2 [M+H]⁺.

Step 8: Preparation of [(2S,5S)-5-(benzyloxymethyl)-1-methyl-pyrrolidin-2-yl]methyl benzoate

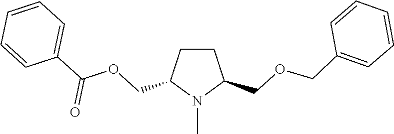 [SMILES: CN1[C@H](COCc2ccccc2)CC[C@H]1COC(=O)c1ccccc1]

To a mixture of [(2S,5S)-5-(benzyloxymethyl) pyrrolidin-2-yl]methyl benzoate (2.75 g, 8.45 mmol, 1.0 eq) and formaldehyde (1.89 mL, 25.35 mmol, 37% purity, 3.0 eq) in i-PrOH (50 mL) and CH₂Cl₂ (50 mL) were added HOAc (1.93 mL, 33.80 mmol, 4.0 eq) and 2-methylpyridine borane (4.52 g, 42.26 mmol, 5.0 eq), and the reaction mixture was stirred at 20° C. for 15 hours. The mixture was concentrated, and the resulting residue was purified by flash chromatography on SiO₂ (gradient: 0-3% CH₃OH in CH₂Cl₂) followed by prep-HPLC (gradient: 20-95% CH₃CN in water (NH₄HCO₃)). The pure fractions were combined and concentrated under reduced pressure, then lyophilized to afford [(2S,5S)-5-(benzyloxymethyl)-1-methyl-pyrrolidin-2-yl]methyl benzoate (1.84 g, 5.42 mmol, 64% yield) as a colorless oil. LC/MS (ESI) m/z: 340.0 [M+H]⁺.

Step 9: Preparation of [(2S,5S)-5-(hydroxymethyl)-1-methyl-pyrrolidin-2-yl]methyl benzoate

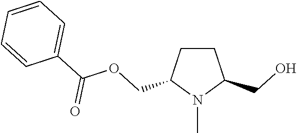 [SMILES: CN1[C@H](CO)CC[C@H]1COC(=O)c1ccccc1]

To a solution of [(2S,5S)-5-(benzyloxymethyl)-1-methyl-pyrrolidin-2-yl]methyl benzoate (940 mg, 2.77 mmol, 1.0 eq) in CH₂Cl₂ (15 mL) at −78° C. was added a solution of BBr₃ (534 uL, 5.54 mmol, 2.0 eq) in CH₂Cl₂ (3 mL), and the reaction mixture was stirred under N₂ at −78° C. for 2 hours. The reaction mixture was quenched with water (10 mL), then concentrated under reduced pressure to remove CH₂Cl₂. The resulting aqueous residue was extracted with MTBE (3×20 mL). The pH of the aqueous layer was adjusted to pH ˜8 by addition of saturated aqueous NaHCO₃, and the resulting aqueous mixture was extracted with 10:1 CH₂Cl₂/CH₃OH (3×20 mL). The combined organic extract was washed with brine (2×20 mL), dried over Na₂SO₄, filtered, and concentrated under reduced pressure. The resulting residue was purified by flash chromatography on SiO₂ (gradient: 0-10% CH₃OH in CH₂Cl₂) to afford [(2S,5S)-5-(hydroxymethyl)-1-methyl-pyrrolidin-2-yl]methyl benzoate (540 mg, 1.84 mmol, 66% yield) as a yellow oil. LC/MS (ESI) m/z: 250.0 [M+H]⁺.

Step 10: Preparation of tert-butyl 3-[[2-[[(2S,5S)-5-(benzoyloxymethyl)-1-methyl-pyrrolidin-2-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

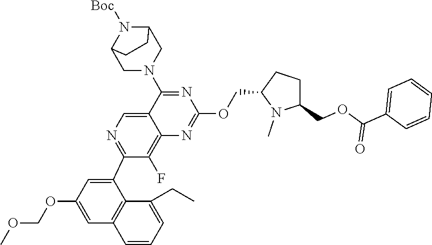 [SMILES: CCc1cccc2cc(OCOC)cc(-c3ncc4c(N5CC6CCC(C5)N6C(=O)OC(C)(C)C)nc(OC[C@@H]5CC[C@@H](COC(=O)c6ccccc6)N5C)nc4c3F)c12]

To a mixture of tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (500 mg, 767.17 umol, 1.0 eq) and [(2S,5S)-5-(hydroxymethyl)-1-methyl-pyrrolidin-2-yl]methyl benzoate (248.6 mg, 997.32 umol, 1.3 eq) in THF (25 mL) was added 4 Å MS (500 mg), and the resulting mixture was cooled to 0° C. t-BuONa (221.2 mg, 2.30 mmol, 3.0 eq) was then added, and the reaction mixture was stirred at 0° C. for 30 minutes. The reaction was quenched by addition of aqueous HCl (2 M) until pH ˜6, and the resulting mixture was extracted with EtOAc (2×30 mL). The organic extracts were combined, dried over Na₂SO₄, filtered, and concentrated under reduced pressure. The resulting residue was purified by flash chromatography on SiO₂ (gradient: 0-2% CH₃OH in CH₂Cl₂) to afford tert-butyl 3-[[2-[[(2S,5S)-5-(benzoyloxymethyl)-1-methyl-pyrrolidin-2-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (488 mg, 285.33 umol, 37% yield) as an orange solid. LC/MS (ESI) m/z: 821.3 [M+H]⁺.

Step 11: Preparation of tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate

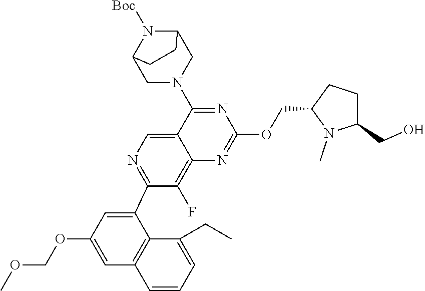 [SMILES: CCc1cccc2cc(OCOC)cc(-c3ncc4c(N5CC6CCC(C5)N6C(=O)OC(C)(C)C)nc(OC[C@@H]5CC[C@@H](CO)N5C)nc4c3F)c12]

To tert-butyl 3-[[2-[[(2S,5S)-5-(benzoyloxymethyl)-1-methyl-pyrrolidin-2-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (388 mg, 226.86 umol, 48% purity, 1.0 eq) in THF (6 mL) was added a solution of LiOH·H₂O (57.12 mg, 1.36 mmol, 6.0 eq) in water (2 mL) at 20° C., and the reaction mixture was stirred at 20° C. for 15 hours followed by 20 hours at at 30° C. The mixture was concentrated under reduced pressure, and the resulting residue was diluted with water (5 mL), adjusting to pH ˜2 by addition of HCl (2 M). EtOAc (30 mL) was then added, the aqueous layer was separated, and the organic layer was further washed with HCl (0.5 M, 3×40 mL). The pH of the combined aqueous extract was adjusted to pH ˜8 by addition of saturated aqueous NaHCO₃. The resulting aqueous mixture was extracted with EtOAc (3×50 mL), and the combined organic extract was washed with brine (2×50 mL), dried over Na₂SO₄, filtered, and concentrated under reduced pressure to afford tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (200 mg, 220.41 umol, 97% yield) as a yellow oil. LC/MS (ESI) m/z: 717.3 [M+H]⁺.

Step 12: Preparation of tert-butyl 3-[7-[8-ethyl-3-(methoxymethoxy)-1-naphthyl]-8-fluoro-2-[[(2S,5S)-5-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate

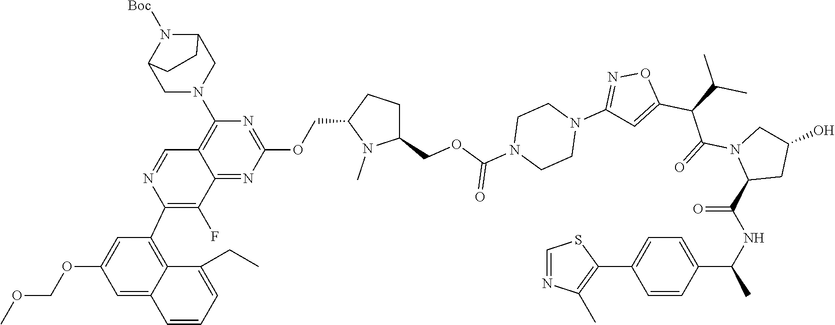 [SMILES: CCc1cccc2cc(OCOC)cc(-c3ncc4c(N5CC6CCC(C5)N6C(=O)OC(C)(C)C)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12]

To tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (200 mg, 220.41 umol, 79% purity, 1.0 eq) in THF (10 mL) were added triethylamine (154 uL, 1.10 mmol, 5.0 eq), DMAP (8.0 mg, 66.12 umol, 0.3 eq), and (4-nitrophenyl) carbonochloridate (85 mg, 418.78 umol, 1.9 eq), and the resulting suspension was stirred at 30° C. for 15 hours. Triethylamine (154 uL, 1.10 mmol, 5.0 eq) and (2S,4R)-4-hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (124.9 mg, 220.41 umol, 1.0 eq) were then added, and the reaction mixture was stirred at 30° C. for 1.5 hours. Additional (2S,4R)-4-hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (149.89 mg, 264.49 umol, 1.2 eq) was added, and the reaction mixture was stirred at 30° C. for 2 hours. The mixture was filtered, and the solvent was concentrated under reduced pressure. The resulting residue was purified by flash chromatography on SiO₂ (gradient: 0-5% CH₃OH in CH₂Cl₂) to afford tert-butyl 3-[7-[8-ethyl-3-(methoxymethoxy)-1-naphthyl]-8-fluoro-2-[[(2S,5S)-5-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate (245 mg, 157.15 umol, 71% yield) as a yellow solid. LC/MS (ESI) m/z: 655.6 [½M+H]⁺

Step 13: Preparation of [(2S,5S)-5-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-1-methylpyrrolidin-2-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

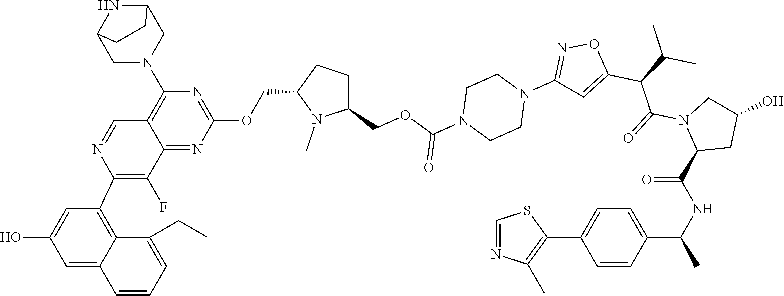 [SMILES: CCc1cccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12]

To tert-butyl 3-[7-[8-ethyl-3-(methoxymethoxy)-1-naphthyl]-8-fluoro-2-[[(2S,5S)-5-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate (245 mg, 157.15 umol, 84% purity, 1.0 eq) in CH₂Cl₂ (8 mL) was added HCl/dioxane (4.0 M, 8 mL, 203.62 eq), and the reaction mixture was stirred at 20° C. for 1 hour. The mixture was diluted with petroleum ether (30 mL), and the resulting yellow precipitate was separated, dissolved with THF (50 mL), and adjusted to pH ˜8 with triethylamine. The resulting mixture was filtered, and the filtrate was concentrated under reduced pressure. The crude product was purified by pre-HPLC (Phenomenex C18 75*30 mm*3 um; mobile phase: [8-48% CH₃CN in water (formic acid)]). The pure fractions were combined and concentrated under reduced pressure, then lyophilized to afford [(2S,5S)-5-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-1-methylpyrrolidin-2-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (147.8 mg, 120.06 umol, 76% yield, formic acid salt) as a white solid. LC/MS (ESI) m/z: 1165.9 [M+H]⁺. ¹H NMR (400 MHz, DMSO-d₆) δ 9.09 (s, 1H), 8.98 (s, 1H), 8.41 (d, J=7.6 Hz, 1H), 7.66 (d, J=8.0 Hz, 1H), 7.47-7.40 (m, 2H), 7.40-7.32 (m, 3H), 7.28 (d, J=2.4 Hz, 1H), 7.12 (d, J=7.2 Hz, 1H), 6.97 (d, J=2.4 Hz, 1H), 6.17 (s, 1H), 4.95-4.87 (m, 1H), 4.50-4.40 (m, 3H), 4.36 (br t, J=7.8 Hz, 1H), 4.29-4.21 (m, 2H), 4.10-3.97 (m, 3H), 3.67-3.57 (m, 11H), 3.25-3.11 (m, 7H), 2.48 (br s, 3H), 2.45 (s, 3H), 2.34-2.11 (m, 4H), 2.05-1.92 (m, 3H), 1.78 (ddd, J=5.2, 7.6, 12.8 Hz, 1H), 1.73-1.57 (m, 6H), 1.47-1.33 (m, 3H), 0.99-0.91 (m, 3H), 0.84-0.75 (m, 6H).

Exemplary Synthesis of (2S,4R)-1-[(2S)-2-(3-{[(3S,7aS)-7a-{[(4-{3,8-diazabicyclo[3.2.1]octan-3-yl}-7-(8-ethynyl-7-fluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 51) and (2S,4R)-1-[(2R)-2-(3-{[(3S,7aS)-7a-{[(4-{3,8-diazabicyclo[3.2.1]octan-3-yl}-7-(8-ethynyl-7-fluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 52)

Step 1: Preparation of 7-fluoronaphthalen-1-ol

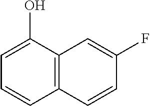 [SMILES: Oc1cccc2ccc(F)cc12]

To a solution of 7-fluorotetralin-1-one (5.00 g, 30.5 mmol, 1 eq) and HBr (747 mg, 3.05 mmol, 0.501 mL, 33% purity, 0.1 eq) in acetic acid (100 mL) at 0° C. was added a solution of Br₂ (5.35 g, 33.5 mmol, 1.73 mL, 1.1 eq) in AcOH (3.5 mL), and the reaction mixture was stirred at 20° C. for 3 hours. The mixture was diluted with dichloromethane (100 mL) and washed with water (3×35 mL). The organic layer was dried over anhydrous sodium sulfate, filtered, and concentrated. The resulting brown oil was dissolved in DMF (50 mL). LiBr (4.50 g, 51.8 mmol, 1.30 mL, 1.7 eq) and Li₂CO₃ (3.83 g, 51.8 mmol, 1.7 eq) were then added, and the reaction mixture was stirred at 160° C. for 5 hours. The mixture was diluted with ethyl acetate (100 mL), then washed with brine (3×35 mL), dried over anhydrous sodium sulfate, filtered, and concentrated. The resulting residue was purified by flash chromatography on SiO₂ (gradient: 0˜12% EtOAc in petroleum ether) to give 7-fluoronaphthalen-1-ol (3.71 g, 22.9 mmol, 75% yield) as a yellow solid. ¹H NMR (400 MHz, CDCl₃) δ 7.85-7.77 (m, 2H), 7.45 (d, J=8.4 Hz, 1H), 7.32-7.24 (m, 2H), 6.84 (d, J=7.6 Hz, 1H), 5.17 (brs, 1H).

Step 2: Preparation of 7-fluoro-8-(2-triisopropylsilylethynyl) naphthalen-1-ol

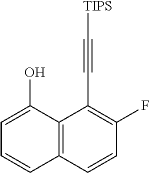

To a solution of 7-fluoronaphthalen-1-ol (2.00 g, 12.3 mmol, 1 eq) and 2-bromoethynyl(triisopropyl)silane (3.87 g, 14.8 mmol, 1.2 eq) in DCE (30 mL) were added NaOAc (202 mg, 2.47 mmol, 0.2 eq), dichloro(p-cymene)ruthenium (ii) dimer (755 mg, 1.23 mmol, 0.1 eq), and K₂CO₃ (1.70 g, 12.3 mmol, 1 eq), and the reaction mixture was stirred at 40° C. under N₂ for 16 hours. The reaction mixture was filtered, and the filtrate was concentrated under reduced pressure. The resulting crude product was purified by flash chromatography on SiO₂ (gradient: 0˜2% EtOAc in petroleum ether) to give 7-fluoro-8-(2-triisopropylsilylethynyl) naphthalen-1-ol (3.45 g, 10.1 mmol, 82% yield) as a yellow solid. LC/MS (ESI) m/z: 343.0 [M+H]⁺. ¹H NMR (400 MHz, CDCl₃) δ 10.11 (s, 1H), 7.93-7.87 (m, 1H), 7.44-7.36 (m, 2H), 7.30 (t, J=8.0 Hz, 1H), 6.95 (d, J=7.6 Hz, 1H), 1.14 (s, 21H).

Step 3: Preparation of [7-fluoro-8-(2-triisopropylsilylethynyl)-1-naphthyl] trifluoromethanesulfonate

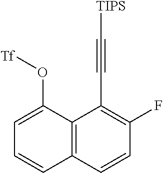

To a solution of 7-fluoro-8-(2-triisopropylsilylethynyl) naphthalen-1-ol (2.05 g, 5.99 mmol, 1 eq) in dichloromethane (20 mL) at −40° C. were added DIEA (928 mg, 7.18 mmol, 1.25 mL, 1.2 eq) and Tf₂O (1.86 g, 6.58 mmol, 1.09 mL, 1.1 eq), and the reaction mixture was stirred at −40° C. under N₂ for 0.5 hour. The reaction was quenched by addition of water (20 mL) and extracted with dichloromethane (2×20 mL). The combined organic extract was dried over anhydrous Na₂SO₄, filtered, and concentrated under reduced pressure. The crude product was triturated with petroleum ether/EtOAc (60 mL/6 mL) at 20° C. for 10 minutes, and the resulting suspension was filtered. The filtrate was concentrated under reduced pressure to give [7-fluoro-8-(2-triisopropylsilylethynyl)-1-naphthyl] trifluoromethanesulfonate (2.81 g, 5.92 mmol, 99% yield) as a yellow solid. ¹H NMR (400 MHz, CDCl₃) δ 7.90-7.80 (m, 2H), 7.59-7.52 (m, 1H), 7.51-7.44 (m, 1H), 7.39 (t, J=8.8 Hz, 1H), 1.30-1.16 (m, 21H).

Step 4: Preparation of 2-[2-fluoro-8-(4,4,5,5-tetramethyl-1,3,2-dioxaborolan-2-yl)-1-naphthyl]ethynyl-triisopropyl-silane

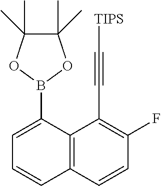

To a solution of [7-fluoro-8-(2-triisopropylsilylethynyl)-1-naphthyl] trifluoromethanesulfonate (3.15 g, 6.64 mmol, 1 eq) and 4,4,5,5-tetramethyl-2-(4,4,5,5-tetramethyl-1,3,2-dioxaborolan-2-yl)-1,3,2-dioxaborolane (4.21 g, 16.6 mmol, 2.5 eq) in dioxane (60 mL) were added KOAc (1.95 g, 19.9 mmol, 3 eq) and Pd(dppf)Cl₂ (486 mg, 0.664 mmol, 0.1 eq), and the reaction mixture was stirred at 110° C. under N₂ for 16 hours. After cooling to room temperature, the reaction mixture was diluted with petroleum ether/EtOAc (300 mL/50 mL). The resulting mixture was filtered, and the filtrate was concentrated under reduced pressure. The crude product was purified by flash chromatography on SiO₂ (gradient: 0˜3% EtOAc in petroleum ether) to give 2-[2-fluoro-8-(4,4,5,5-tetramethyl-1,3,2-dioxaborolan-2-yl)-1-naphthyl]ethynyl-triisopropyl-silane (1.74 g, 3.85 mmol, 58% yield) as a yellow solid. ¹H NMR (400 MHz, CDCl₃) δ 7.86-7.74 (m, 3H), 7.45-7.40 (m, 1H), 7.27-7.24 (m, 1H), 1.44 (s, 12H), 1.21-1.14 (m, 21H).

Step 5: Preparation of tert-butyl 3-[8-fluoro-7-[7-fluoro-8-(2-triisopropylsilylethynyl)-1-naphthyl]-2-methylsulfanyl-pyrido[4,3-d]pyrimidin-4-yl]-3,8-diazabicyclo[3.2.1]octane-8-carboxylate

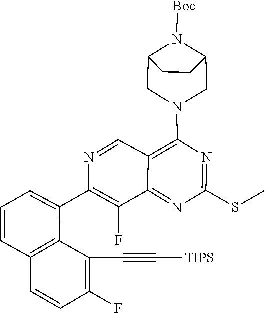

To a solution of tert-butyl 3-(7-chloro-8-fluoro-2-methylsulfanyl-pyrido[4,3-d]pyrimidin-4-yl)-3,8-diazabicyclo[3.2.1]octane-8-carboxylate (1.80 g, 4.09 mmol, 1 eq) and 2-[2-fluoro-8-(4,4,5,5-tetramethyl-1,3,2-dioxaborolan-2-yl)-1-naphthyl]ethynyl-triisopropyl-silane (2.04 g, 4.50 mmol, 1.1 eq) in dioxane (30 mL) were added K₃PO₄ (3 M, 4.09 mL, 3 eq) and Catacxium A Pd G3 (596 mg, 0.818 mmol, 0.2 eq), and the reaction mixture was stirred at 100° C. under N₂ (degassed under vacuum and purged with N₂ several times) for 16 hours. After cooling to room temperature, the reaction mixture was quenched by addition of water (30 mL) and extracted with EtOAc (3×30 mL). The combined organic extract was washed with brine (30 mL), dried over anhydrous Na₂SO₄, filtered, and concentrated. The crude product was purified by flash chromatography on SiO₂ (gradient: 0˜15% tetrahydrofuran in petroleum ether) to give tert-butyl 3-[8-fluoro-7-[7-fluoro-8-(2-triisopropylsilylethynyl)-1-naphthyl]-2-methylsulfanyl-pyrido[4,3-d]pyrimidin-4-yl]-3,8-diazabicyclo[3.2.1]octane-8-carboxylate (2.99 g, 4.10 mmol, 100% yield) as a gray solid. LC/MS (ESI) m/z: 730.6 [M+H]⁺.

Step 6: Preparation of tert-butyl 3-[8-fluoro-7-[7-fluoro-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate

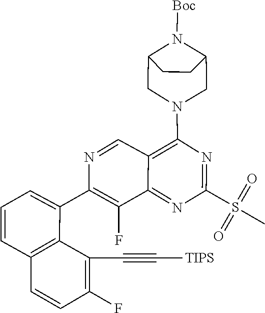

To a solution of tert-butyl 3-[8-fluoro-7-[7-fluoro-8-(2-triisopropylsilylethynyl)-1-naphthyl]-2-methylsulfanyl-pyrido[4,3-d]pyrimidin-4-yl]-3,8-diazabicyclo[3.2.1]octane-8-carboxylate (3.10 g, 4.25 mmol, 1 eq) in DMF (50 mL) was added 4 Å MS (5.0 g), and the reaction mixture was stirred at 40° C. for 8 hours. Oxone (7.83 g, 12.7 mmol, 3 eq) was then added, and the reaction mixture was stirred at 40° C. for 16 hours. The reaction was quenched by slow addition of a solution of Na₂S₂O₃ (3.36 g, 21.2 mmol, 5 eq)/NaHCO₃ (1.78 g, 21.2 mmol, 5 eq) in H₂O (80 mL). EtOAc (80 mL) was then added, and the resulting mixture was filtered. The filtrate was separated, and the aqueous layer was extracted with EtOAc (2×80 mL). The combined organic extract was washed with brine (3×100 mL), dried over anhydrous Na₂SO₄, filtered, and concentrated. The crude product was purified by flash chromatography on SiO₂ (gradient: 0˜30% EtOAc in petroleum ether) to give tert-butyl 3-[8-fluoro-7-[7-fluoro-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate (2.85 g, 3.74 mmol, 88% yield) as a yellow solid. LC/MS (ESI) m/z: 762.2 [M+H]⁺.

Step 7: Preparation of tert-butyl 3-[8-fluoro-7-[7-fluoro-8-(2-triisopropylsilylethynyl)-1-naphthyl]-2-[[(3S,8S)-3-[[5-(1-methoxycarbonyl-2-methyl-propyl)isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

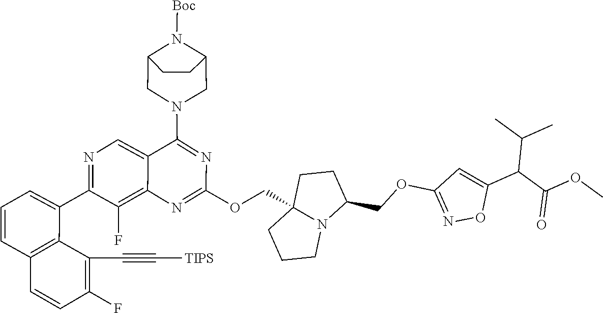

To a solution of tert-butyl 3-[8-fluoro-7-[7-fluoro-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate (750 mg, 0.984 mmol, 1 eq) and methyl 2-[3-[[(3S,8S)-8-(hydroxymethyl)-1,2,3,5,6,7-hexahydropyrrolizin-3-yl]methoxy]isoxazol-5-yl]-3-methyl-butanoate (302 mg, 0.856 mmol, 0.87 eq) in toluene (10 mL) were added DIEA (127 mg, 0.984 mmol, 0.171 mL, 1 eq) and 4 Å MS (1.0 g), and the reaction mixture was stirred at 20° C. for 0.5 hour. t-BuONa (189 mg, 1.97 mmol, 2 eq) was then added at 0° C., and the reaction mixture was stirred at 0° C. for 0.5 hour. The reaction mixture was quenched by addition of water/2N aqueous HCl (20 mL/0.4 mL), then extracted with EtOAc (3×30 mL). The combined organic extract was washed with brine (50 mL), dried over anhydrous Na₂SO₄, filtered, and concentrated. The crude product was purified by flash chromatography on SiO₂ (gradient: 0˜55% EtOAc in petroleum ether) to give tert-butyl 3-[8-fluoro-7-[7-fluoro-8-(2-triisopropylsilylethynyl)-1-naphthyl]-2-[[(3S,8S)-3-[[5-(1-methoxycarbonyl-2-methyl-propyl)isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (606 mg, 0.586 mmol, 60% yield) as a yellow solid. LC/MS (ESI) m/z: 1034.4 [M+H]⁺.

Step 8: Preparation of 2-[3-[[(3S,8S)-8-[[4-(8-tert-butoxycarbonyl-3,8-diazabicyclo[3.2.1]octan-3-yl)-8-fluoro-7-[7-fluoro-8-(2-triisopropylsilylethynyl)-1-naphthyl]pyrido[4,3-d]pyrimidin-2-yl]oxymethyl]-1,2,3,5,6,7-hexahydropyrrolizin-3-yl]methoxy]isoxazol-5-yl]-3-methyl-butanoic acid

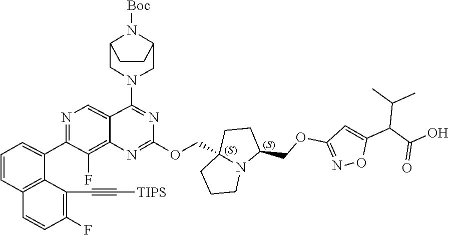

To a solution of tert-butyl 3-[8-fluoro-7-[7-fluoro-8-(2-triisopropylsilylethynyl)-1-naphthyl]-2-[[(3S,8S)-3-[[5-(1-methoxycarbonyl-2-methyl-propyl)isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (600 mg, 0.580 mmol, 1 eq) in THF (3 mL) was added a solution of LiOH·H₂O (73 mg, 1.7 mmol, 3 eq) in water (3 mL), and the reaction mixture was stirred at 20° C. for 16 hours. The reaction mixture was diluted with water (15 mL), and the pH of the resulting aqueous mixture was adjusted to pH=5 by addition of 2N aqueous HCl. The mixture was extracted with EtOAc (3×15 mL), and the combined organic extract was washed with brine (20 mL), dried over anhydrous Na₂SO₄, filtered, and concentrated under reduced pressure to give 2-[3-[[(3S,8S)-8-[[4-(8-tert-butoxycarbonyl-3,8-diazabicyclo[3.2.1]octan-3-yl)-8-fluoro-7-[7-fluoro-8-(2-triisopropylsilylethynyl)-1-naphthyl]pyrido[4,3-d]pyrimidin-2-yl]oxymethyl]-1,2,3,5,6,7-hexahydropyrrolizin-3-yl]methoxy]isoxazol-5-yl]-3-methyl-butanoic acid (580 mg, 0.568 mmol, 98% yield) as a yellow solid. LC/MS (ESI) m/z: 1020.5 [M+H].

Step 9: Preparation of (1R,5S)-tert-butyl 3-(8-fluoro-7-(7-fluoro-8-((triisopropylsilyl) ethynyl) naphthalen-1-yl)-2-(((3S,7aS)-3-(((5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)oxy)methyl) hexahydro-1H-pyrrolizin-7a-yl)methoxy)pyrido[4,3-d]pyrimidin-4-yl)-3,8-diazabicyclo[3.2.1]octane-8-carboxylate

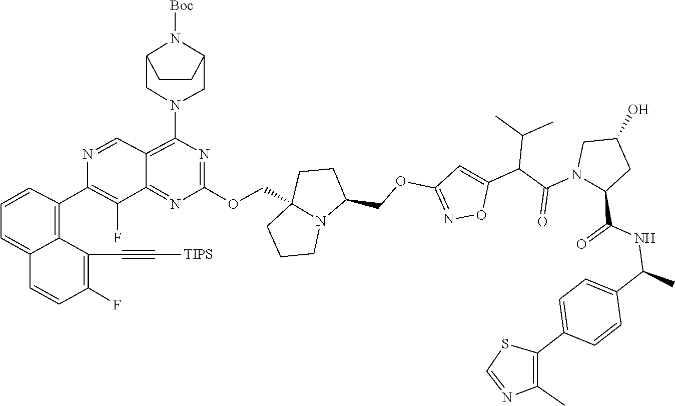

To a solution of 2-[3-[[(3S,8S)-8-[[4-(8-tert-butoxycarbonyl-3,8-diazabicyclo[3.2.1]octan-3-yl)-8-fluoro-7-[7-fluoro-8-(2-triisopropylsilylethynyl)-1-naphthyl]pyrido[4,3-d]pyrimidin-2-yl]oxymethyl]-1,2,3,5,6,7-hexahydropyrrolizin-3-yl]methoxy]isoxazol-5-yl]-3-methyl-butanoic acid (580 mg, 0.568 mmol, 1 eq) in dichloromethane (20 mL) were added DIEA (735 mg, 5.68 mmol, 10 eq), HATU (259 mg, 0.682 mmol, 1.2 eq), and (2S,4R)-4-hydroxy-N-[(1S)-1-[4-(4-methylthiazol-5-yl)phenyl]ethyl]pyrrolidine-2-carboxamide (640 mg, 1.48 mmol, 85% purity, 2.6 eq, HCl), and the reaction mixture was stirred at 20° C. for 1 hour. The mixture was poured onto water (20 mL) and the layers were separated. The aqueous layer was extracted with dichloromethane (20 mL). The combined organic extract was washed with brine (20 mL), dried over anhydrous Na₂SO₄, filtered, and concentrated. The resulting crude product was purified by flash chromatography on SiO₂ (gradient: 0˜4% (10% 7 M NH₃/CH₃OH) in chloromethane) to give (1R,5S)-tert-butyl 3-(8-fluoro-7-(7-fluoro-8-((triisopropylsilyl) ethynyl) naphthalen-1-yl)-2-(((3S,7aS)-3-(((5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)oxy)methyl) hexahydro-1H-pyrrolizin-7a-yl)methoxy)pyrido[4,3-d]pyrimidin-4-yl)-3,8-diazabicyclo[3.2.1]octane-8-carboxylate (661 mg, 0.496 mmol, 87% yield) as a yellow solid. LC/MS (ESI) m/z: 1333.7 [M+H]⁺.

Step 10: Preparation of (1R,5S)-tert-butyl 3-(7-(8-ethynyl-7-fluoronaphthalen-1-yl)-8-fluoro-2-(((3S,7aS)-3-(((5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)oxy)methyl) hexahydro-1H-pyrrolizin-7a-yl)methoxy)pyrido[4,3-d]pyrimidin-4-yl)-3,8-diazabicyclo[3.2.1]octane-8-carboxylate

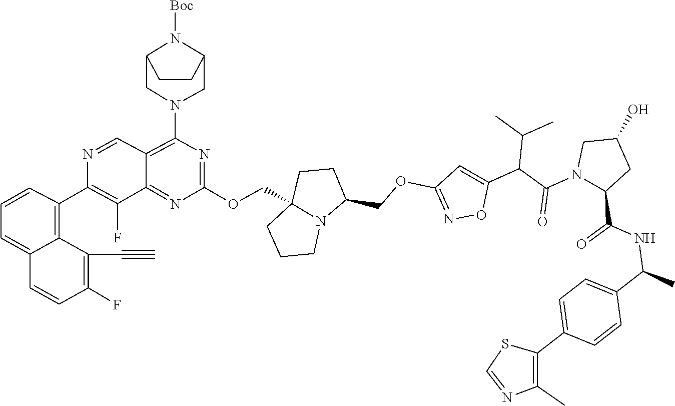 [SMILES: C#Cc1c(F)ccc2cccc(-c3ncc4c(N5CC6CCC(C5)N6C(=O)OC(C)(C)C)nc(OC[C@@]56CCCN5[C@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

To a solution of (1R,5S)-tert-butyl 3-(8-fluoro-7-(7-fluoro-8-((triisopropylsilyl) ethynyl) naphthalen-1-yl)-2-(((3S,7aS)-3-(((5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)oxy)methyl) hexahydro-1H-pyrrolizin-7a-yl)methoxy)pyrido[4,3-d]pyrimidin-4-yl)-3,8-diazabicyclo[3.2.1]octane-8-carboxylate (259 mg, 0.194 mmol, 1 eq) in DMF (2.5 mL) were added CsF (251 mg, 1.65 mmol, 8.5 eq), and the reaction mixture was stirred at 20° C. for 16 hours. The reaction mixture was quenched by addition of water (50 mL) and the resulting suspension was filtered. The cake was dissolved in CH₂Cl₂ (30 mL) and the organic layer was dried over anhydrous Na₂SO₄, filtered and concentrated under reduced pressure to give (1R,5S)-tert-butyl 3-(7-(8-ethynyl-7-fluoronaphthalen-1-yl)-8-fluoro-2-(((3S,7aS)-3-(((5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)oxy)methyl) hexahydro-1H-pyrrolizin-7a-yl)methoxy)pyrido[4,3-d]pyrimidin-4-yl)-3,8-diazabicyclo[3.2.1]octane-8-carboxylate (225 mg, 0.191 mmol, 98% yield) as a yellow solid. LC/MS (ESI) m/z: 589.5 [½M+H].

Step 11: Preparation of (2S,4R)-1-[(2S)-2-(3-{[(3S,7aS)-7a-{[(4-{3,8-diazabicyclo[3.2.1]octan-3-yl}-7-(8-ethynyl-7-fluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide and (2S,4R)-1-[(2R)-2-(3-{[(3S,7aS)-7a-{[(4-{3,8-diazabicyclo[3.2.1]octan-3-yl}-7-(8-ethynyl-7-fluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide

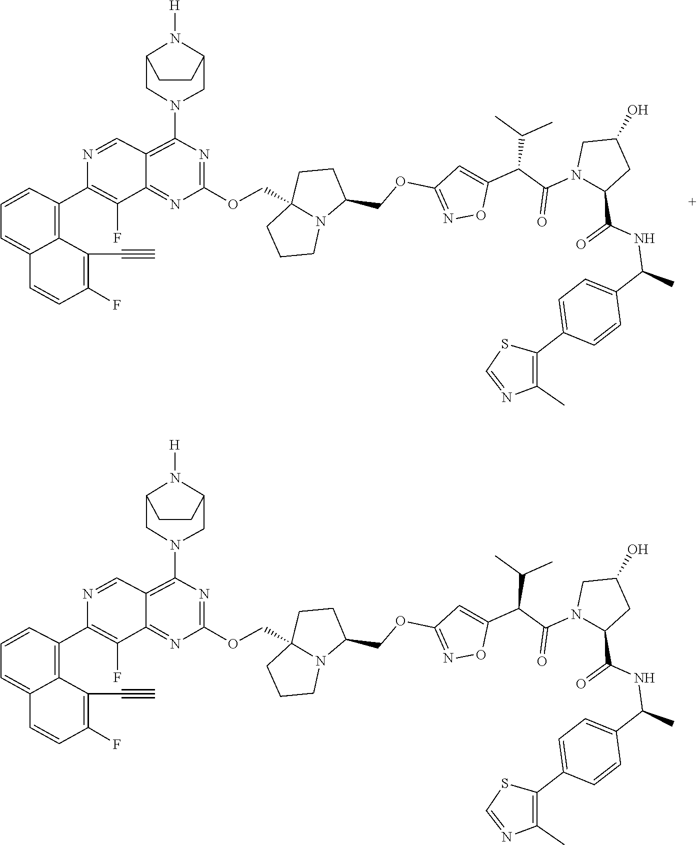 [SMILES: C#Cc1c(F)ccc2cccc(-c3ncc4c(N5CC6CCC(C5)N6)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12 ; C#Cc1c(F)ccc2cccc(-c3ncc4c(N5CC6CCC(C5)N6)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

To a solution of (1R,5S)-tert-butyl 3-(7-(8-ethynyl-7-fluoronaphthalen-1-yl)-8-fluoro-2-(((3S,7aS)-3-(((5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)oxy)methyl) hexahydro-1H-pyrrolizin-7a-yl)methoxy)pyrido[4,3-d]pyrimidin-4-yl)-3,8-diazabicyclo[3.2.1]octane-8-carboxylate (463 mg, 0.393 mmol, 1 eq) in dichloromethane (2 mL) was added 4 M HCl/dioxane (2 mL), and the suspension was stirred at 20° C. for 0.5 hour. The reaction mixture was diluted with petroleum ether (40 mL) and the resulting suspension was filtered. The cake was poured onto water/saturated aqueous NaHCO₃ (25 mL, v/v=4/1), and the resulting mixture was extracted with 4:1 EtOAc/THF (3×25 mL). The combined organic extract was dried over anhydrous Na₂SO₄, filtered, and concentrated under reduced pressure. The crude mixture was separated by SFC (column: DAICEL CHIRALPAK IA (250 mm*30 mm, 10 um); mobile phase: [30% CH₃CN in EtOH (0.1% NH₄OH)]) to give the desired products.

(2S,4R)-1-[(2S)-2-(3-{[(3S,7aS)-7a-{[(4-{3,8-diazabicyclo[3.2.1]octan-3-yl}-7-(8-ethynyl-7-fluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (141.1 mg, 0.131 mmol, 35% yield) was obtained as a yellow solid. LC/MS (ESI) m/z: 1077.3 [M+H]⁺. ¹H NMR (400 MHz, CD₃OD) δ 9.04 (s, 1H), 8.90-8.83 (m, 1H), 8.16-8.06 (m, 2H), 7.70-7.61 (m, 2H), 7.50-7.31 (m, 5H), 6.10-5.98 (m, 1H), 5.13-4.93 (m, 1H), 4.67-4.54 (m, 3H), 4.46-4.40 (m, 3H), 4.36-4.28 (m, 1H), 3.84-3.57 (m, 8H), 3.46-3.41 (m, 1H), 3.10-2.85 (m, 2H), 2.53-2.43 (m, 3H), 2.42-2.32 (m, 1H), 2.29-2.17 (m, 2H), 2.15-1.69 (m, 13H), 1.60-1.43 (m, 3H), 1.09-0.93 (m, 3H), 0.93-0.82 (m, 3H).

(2S,4R)-1-[(2R)-2-(3-{[(3S,7aS)-7a-{[(4-{3,8-diazabicyclo[3.2.1]octan-3-yl}-7-(8-ethynyl-7-fluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (166.4 mg, 0.154 mmol, 38% yield) was obtained as a yellow solid. LC/MS (ESI) m/z: 1077.3 [M+H]⁺. ¹H NMR (400 MHz, CD₃OD) δ 9.03 (s, 1H), 8.87 (s, 1H), 8.15-8.06 (m, 2H), 7.70-7.59 (m, 2H), 7.48-7.35 (m, 5H), 6.05-5.96 (m, 1H), 5.03 (q, J=6.8 Hz, 1H), 4.67-4.57 (m, 2H), 4.53-4.48 (m, 1H), 4.44-4.34 (m, 3H), 4.32-4.25 (m, 1H), 3.83 (dd, J=10.8, 4.0 Hz, 1H), 3.78-3.52 (m, 7H), 3.48-3.40 (m, 1H), 3.02-2.82 (m, 2H), 2.50-2.42 (m, 3H), 2.42-2.29 (m, 1H), 2.27-2.13 (m, 2H), 2.12-1.65 (m, 13H), 1.60-1.47 (m, 3H), 1.05 (d, J=6.8 Hz, 3H), 0.93-0.86 (m, 3H).

Exemplary Synthesis of [(3S,7aR)-7a-{[(4-{3,8-diazabicyclo[3.2.1]octan-3-yl}-7-(8-ethynyl-7-fluoronaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 53)

Step 1: Preparation of tert-butyl 3-[[2-[[(3S,8R)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate

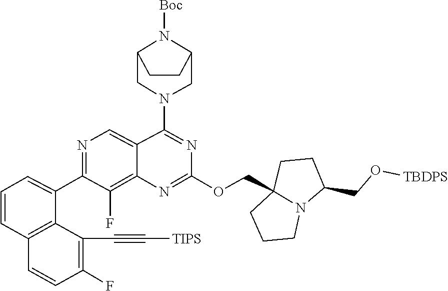

To a solution of tert-butyl 3-[8-fluoro-7-[7-fluoro-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate (500 mg, 0.656 mmol, 1 eq) and [(3S,8R)-3-[[tert-butyl(diphenyl)silyl]oxymethyl]-1,2,3,5,6,7-hexahydropyrrolizin-8-yl]methanol (269 mg, 0.656 mmol, 1 eq) in toluene (10 mL) at 0° C. was added t-BuONa (126 mg, 1.31 mmol, 2 eq), and the reaction mixture was stirred at 0° C. under N₂ for 0.5 hour. The reaction mixture was quenched by addition of water/2N aqueous HCl (20 mL/0.3 mL) and extracted with EtOAc (3×30 mL). The combined organic extract was washed with brine (2×50 mL), dried over anhydrous Na₂SO₄, filtered, and concentrated. The crude product was purified by flash chromatography on SiO₂ (gradient: 0˜50% EtOAc in petroleum ether) to give tert-butyl 3-[[2-[[(3S,8R)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate (285 mg, 0.261 mmol, 40% yield) as a yellow solid. LC/MS (ESI) m/z: 1091.5 [M+H].

Step 2: Preparation of tert-butyl 3-[7-(8-ethynyl-7-fluoro-1-naphthyl)-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

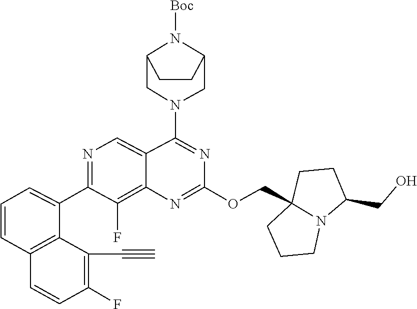 [SMILES: C#Cc1c(F)ccc2cccc(-c3ncc4c(N5CC6CCC(C5)N6C(=O)OC(C)(C)C)nc(OC[C@]56CCCN5[C@H](CO)CC6)nc4c3F)c12]

To a solution of tert-butyl 3-[[2-[[(3S,8R)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate (286 mg, 0.262 mmol, 1 eq) in DMF (3 mL) was added CsF (318 mg, 2.10 mmol, 8 eq), and the reaction mixture was stirred at 30° C. for 40 hours. The reaction mixture was diluted CH₂Cl₂ (50 mL) and filtered. The filtrate was concentrated under reduced, and the crude product was purified by flash chromatography on SiO₂ (gradient: 0˜4% (10% 7 M NH₃/CH₃OH) in dichloromethane) to give tert-butyl 3-[7-(8-ethynyl-7-fluoro-1-naphthyl)-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (146 mg, 0.210 mmol, 80% yield) as a yellow solid. LC/MS (ESI) m/z: 697.2 [M+H]⁺.

Step 3: Preparation of tert-butyl 3-[7-(8-ethynyl-7-fluoro-1-naphthyl)-8-fluoro-2-[[(3S,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

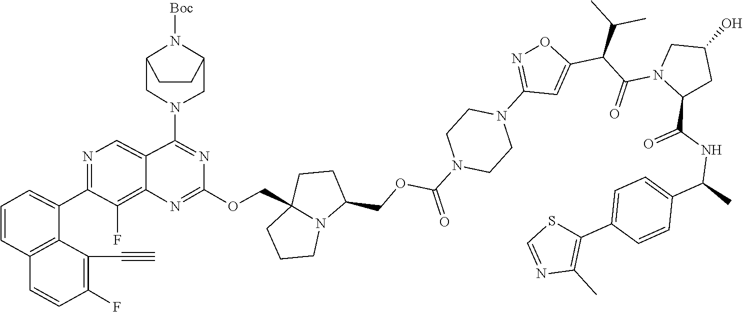 [SMILES: C#Cc1c(F)ccc2cccc(-c3ncc4c(N5CC6CCC(C5)N6C(=O)OC(C)(C)C)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-(8-ethynyl-7-fluoro-1-naphthyl)-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (146 mg, 0.210 mmol, 1 eq) in THF (5 mL) were added triethylamine (212 mg, 2.10 mmol, 292 μL, 10 eq), DMAP (2.5 mg, 0.021 mmol, 0.1 eq), and 4-nitrophenyl carbonochloridate (80 mg, 0.40 mmol, 1.9 eq), and the reaction mixture was stirred at 20° C. for 16 hours. (2S,4R)-4-Hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (178 mg, 0.314 mmol, 1.5 eq) was then added, and the reaction mixture was stirred at 20° C. for 16 hour. The reaction mixture was filtered, and the filtrate was concentrated under reduced pressure. The crude product was purified by flash chromatography on SiO₂ (gradient: 0˜4% (10% 7 M NH₃/CH₃OH) in dichloromethane) to give tert-butyl 3-[7-(8-ethynyl-7-fluoro-1-naphthyl)-8-fluoro-2-[[(3S,8R)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (241 mg, 0.187 mmol, 89% yield) as a yellow solid. LC/MS (ESI) m/z: 645.5 [½M+H]⁺.

Step 4: Preparation of [(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

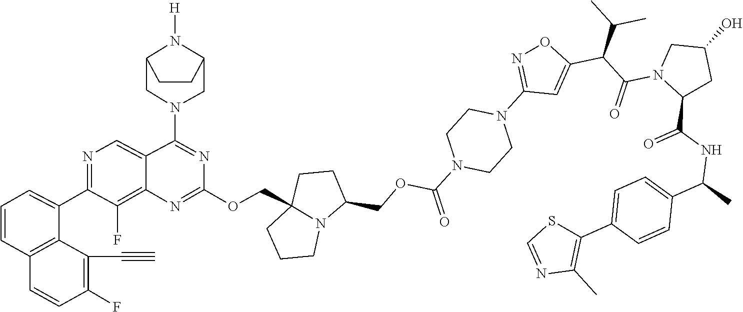 [SMILES: C#Cc1c(F)ccc2cccc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-(8-ethynyl-7-fluoro-1-naphthyl)-8-fluoro-2-[[(3S,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (240 mg, 0.186 mmol, 1 eq) in CH₂Cl₂ (2 mL) was added 4 M HCl/dioxane (2 mL), and the suspension was stirred at 20° C. for 0.5 hour. The reaction mixture was diluted with petroleum ether (40 mL), and the resulting suspension was filtered. The cake was poured into 4:1 water/saturated aqueous NaHCO₃ (25 mL), and the resulting mixture was extracted with 4:1 EtOAc/THF (3×25 mL). The combined organic extract was washed with brine (30 mL), dried over anhydrous Na₂SO₄, filtered, and concentrated. The resulting residue was purified by prep-HPLC (column: DAICEL CHIRALPAK IC (250 mm*30 mm, 10 um); mobile phase: [45% CH₃CN in EtOH (0.1% NH₄OH)]) to give [(3S,7aR)-7a-{[(4-{3,8-diazabicyclo[3.2.1]octan-3-yl}-7-(8-ethynyl-7-fluoronaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (172.6 mg, 0.145 mmol, 78% yield, 99.77% purity) as a yellow solid. LC/MS (ESI) m/z: 1189.3 [M+H]⁺. ¹H NMR (400 MHz, CD₃OD) δ 9.08-8.99 (m, 1H), 8.89-8.85 (m, 1H), 8.16-8.06 (m, 2H), 7.69-7.61 (m, 2H), 7.48-7.32 (m, 5H), 6.13-6.06 (m, 1H), 5.03 (q, J=6.8 Hz, 1H), 4.75-4.49 (m, 4H), 4.30-4.19 (m, 2H), 4.15-4.08 (m, 1H), 4.04-3.96 (m, 1H), 3.87-3.79 (m, 1H), 3.79-3.51 (m, 10H), 3.26-3.16 (m, 4H), 3.11-2.98 (m, 2H), 2.88-2.75 (m, 1H), 2.50-2.43 (m, 3H), 2.42-2.28 (m, 1H), 2.25-2.12 (m, 2H), 2.09-1.61 (m, 13H), 1.60-1.47 (m, 3H), 1.05 (d, J=6.4 Hz, 3H), 0.93-0.85 (m, 3H).

Exemplary Synthesis of (2S,4R)-1-[(2R)-2-(3-{[(3S,7aS)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 54)

Step 1: Preparation of tert-butyl 3-[7-(8-ethyl-1-naphthyl)-8-fluoro-2-[[(3S,8S)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

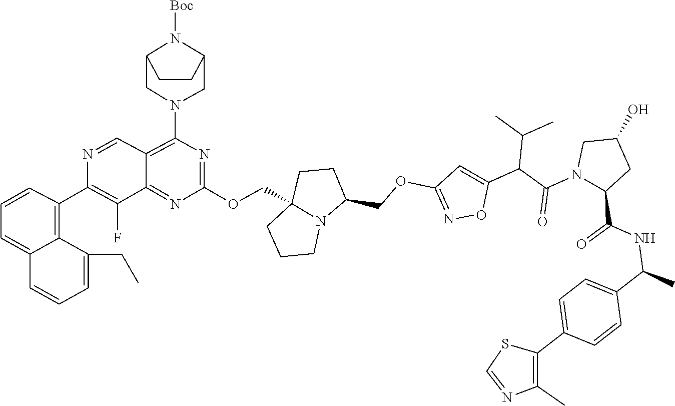 [SMILES: CCc1cccc2cccc(-c3ncc4c(N5CC6CCC(C5)N6C(=O)OC(C)(C)C)nc(OC[C@@]56CCCN5[C@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to (1R,5S)-tert-butyl 3-(7-(8-ethynyl-7-fluoronaphthalen-1-yl)-8-fluoro-2-(((3S,7aS)-3-(((5-(1-((2S,4R)-4-hydroxy-2-(((S)-1-(4-(4-methylthiazol-5-yl)phenyl)ethyl) carbamoyl) pyrrolidin-1-yl)-3-methyl-1-oxobutan-2-yl)isoxazol-3-yl)oxy)methyl) hexahydro-1H-pyrrolizin-7a-yl)methoxy)pyrido[4,3-d]pyrimidin-4-yl)-3,8-diazabicyclo[3.2.1]octane-8-carboxylate starting from methyl 2-[3-[[(3S,8S)-8-(hydroxymethyl)-1,2,3,5,6,7-hexahydropyrrolizin-3-yl]methoxy]isoxazol-5-yl]-3-methyl-butanoate and tert-butyl 3-[7-(8-ethyl-1-naphthyl)-8-fluoro-2-methylsulfonyl-pyrido[4,3-d]pyrimidin-4-yl]-3,8-diazabicyclo[3.2.1]octane-8-carboxylate and purified by flash chromatography on SiO₂ (gradient: 0-4% CH₃OH in CH₂Cl₂) to afford tert-butyl 3-[7-(8-ethyl-1-naphthyl)-8-fluoro-2-[[(3S,8S)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (480 mg, 391.95 umol, 50% yield) as a yellow solid. LC/MS (ESI) m/z: 1163.6 [M+H]⁺.

Step 2: Preparation of tert-butyl 3-[7-(8-ethyl-1-naphthyl)-8-fluoro-2-[[(3S,8S)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate & tert-butyl 3-[7-(8-ethyl-1-naphthyl)-8-fluoro-2-[[(3S,8S)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

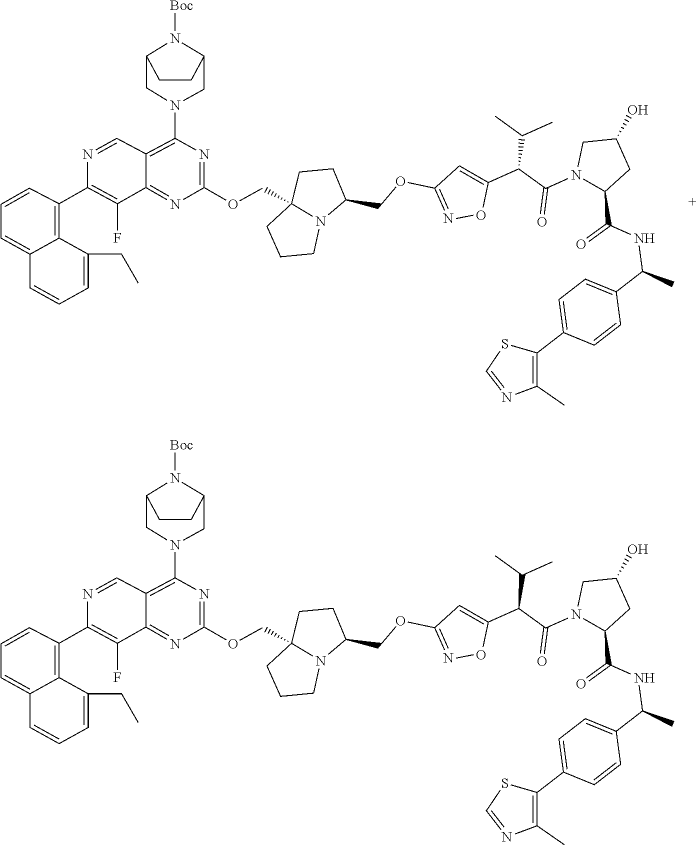 [SMILES: CCc1cccc2cccc(-c3ncc4c(N5CC6CCC(C5)N6C(=O)OC(C)(C)C)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12 ; CCc1cccc2cccc(-c3ncc4c(N5CC6CCC(C5)N6C(=O)OC(C)(C)C)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

Tert-butyl 3-[7-(8-ethyl-1-naphthyl)-8-fluoro-2-[[(3S,8S)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (480 mg, 412.58 umol, 1.0 eq) was separated by SFC (column: DAICEL CHIRALPAK IA (250 mm*30 mm, 10 um); mobile phase: [30% CH₃CN in EtOH (0.1% NH₄OH)]). The pure fractions were combined and concentrated under reduced pressure to afford the desired products.

tert-Butyl 3-[7-(8-ethyl-1-naphthyl)-8-fluoro-2-[[(3S,8S)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (180 mg, 143.42 umol, 35% yield) as a yellow solid. LC/MS (ESI) m/z: 1163.5 [M+H]⁺.

tert-Butyl 3-[7-(8-ethyl-1-naphthyl)-8-fluoro-2-[[(3S,8S)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (250 mg, 206.25 umol, 50% yield) as a yellow solid. LC/MS (ESI) m/z: 1163.6 [M+H]⁺.

Step 3: Preparation of (2S,4R)-1-[(2R)-2-(3-{[(3S,7aS)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide

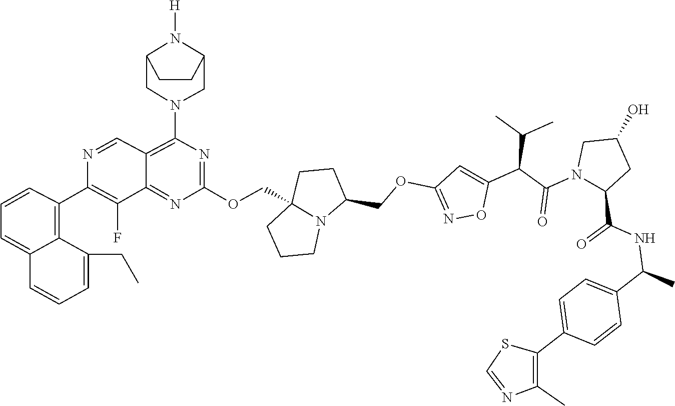 [SMILES: CCc1cccc2cccc(-c3ncc4c(N5CC6CCC(C5)N6)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

To tert-butyl 3-[7-(8-ethyl-1-naphthyl)-8-fluoro-2-[[(3S,8S)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (250 mg, 206.25 umol, 95.98% purity, 1.0 eq) in CH₂Cl₂ (5 mL) was added HCl/dioxane (4 M, 5 mL, 96.97 eq), and the reaction mixture was stirred at 20° C. for 0.5 hour. The reaction mixture was diluted with petroleum ether (5 mL), and the resulting yellow precipitate was separated, then dissolved with CH₂Cl₂ (10 mL). triethylamine was added, and the resulting mixture was concentrated under reduced pressure. The crude product was purified by prep-HPLC (column: Phenomenex C18 75*30 mm*3 um; mobile phase: [12-42% CH₃CN in water (formic acid)]).

The pure fractions were combined and concentrated under reduced pressure, then lyophilized to afford (2S,4R)-1-[(2R)-2-(3-{[(3S,7aS)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (156.8 mg, 140.74 umol, 68% yield, formic acid salt) as a white solid. LC/MS (ESI) m/z: 1063.9 [M+H]⁺. ¹H NMR (400 MHz, DMSO-d₆) δ 9.12 (s, 1H), 8.98 (s, 1H), 8.44 (d, J=7.6 Hz, 1H), 8.08 (dd, J=1.2, 8.4 Hz, 1H), 7.91 (d, J=8.0 Hz, 1H), 7.58 (t, J=7.6 Hz, 1H), 7.54-7.48 (m, 1H), 7.46-7.41 (m, 3H), 7.40-7.32 (m, 3H), 6.11 (d, J=3.2 Hz, 1H), 4.91 (qd, J=6.8, 13.6 Hz, 2H), 4.61-4.42 (m, 3H), 4.40-4.34 (m, 2H), 4.33-4.27 (m, 2H), 4.20 (br dd, J=4.4, 10.4 Hz, 1H), 4.15-4.08 (m, 1H), 3.85-3.75 (m, 3H), 3.74-3.60 (m, 3H), 3.50-3.42 (m, 2H), 2.82-2.73 (m, 2H), 2.45 (s, 3H), 2.39-2.21 (m, 3H), 2.12-1.98 (m, 2H), 1.85-1.69 (m, 11H), 1.60-1.50 (m, 1H), 1.46-1.35 (m, 3H), 0.99-0.92 (m, 3H), 0.86-0.77 (m, 6H).

Exemplary Synthesis of (2S,4R)-1-[(2S)-2-(3-{[(3S,7aS)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 55)

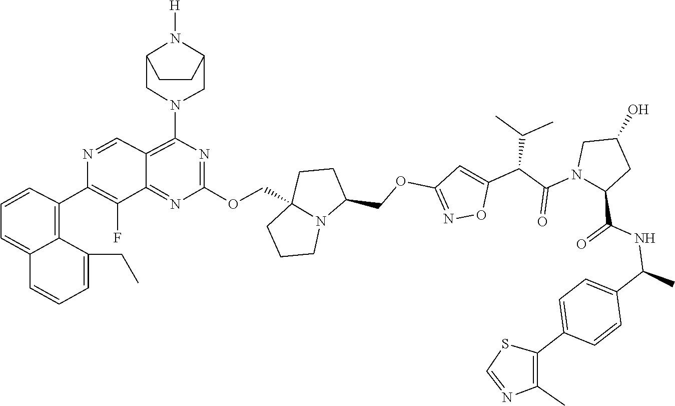 [SMILES: CCc1cccc2cccc(-c3ncc4c(N5CC6CCC(C5)N6)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 54 from tert-Butyl 3-[7-(8-ethyl-1-naphthyl)-8-fluoro-2-[[(3S,8S)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate, and purified by prep-HPLC (column: Phenomenex C18 75*30 mm*3 um; mobile phase: [10-40% CH₃CN in water (formic acid)]). The pure fractions were combined and concentrated under reduced pressure, then lyophilized to afford (2S,4R)-1-[(2S)-2-(3-{[(3S,7aS)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (103.9 mg, 92.28 umol, 64% yield, formic acid salt) as a white solid. LC/MS (ESI) m/z: 1063.8 [M+H]⁺. ¹H NMR (400 MHz, DMSO-d₆) δ 9.10 (s, 1H), 9.00-8.92 (m, 1H), 8.31 (d, J=7.6 Hz, 1H), 8.07 (d, J=8.0 Hz, 1H), 7.91 (d, J=8.0 Hz, 1H), 7.61-7.55 (m, 1H), 7.53-7.26 (m, 7H), 6.20-6.08 (m, 1H), 4.94-4.83 (m, 1H), 4.53-4.38 (m, 4H), 4.38-4.30 (m, 2H), 4.27 (br dd, J=5.2, 10.4 Hz, 2H), 4.16 (br dd, J=4.0, 10.4 Hz, 1H), 4.06 (br d, J=10.4 Hz, 1H), 3.76 (br d, J=8.4 Hz, 1H), 3.67 (br d, J=7.6 Hz, 4H), 3.55-3.38 (m, 3H), 2.78-2.65 (m, 2H), 2.47-2.42 (m, 3H), 2.40-2.21 (m, 3H), 2.13-1.98 (m, 2H), 1.87-1.59 (m, 11H), 1.56-1.47 (m, 1H), 1.47-1.30 (m, 3H), 1.00-0.68 (m, 9H).

Exemplary Synthesis of [(3S,7aR)-7a-{[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 56)

Step 1: Preparation of tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate

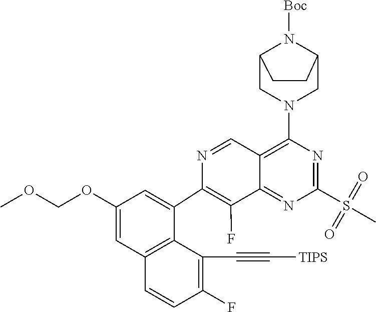

A solution of tert-butyl 3-[8-fluoro-7-[7-fluoro-3-(methoxymethoxy)-8-(2-triisopropylsilylethynyl)-1-naphthyl]-2-methylsulfanyl-pyrido[4,3-d]pyrimidin-4-yl]-3,8-diazabicyclo[3.2.1]octane-8-carboxylate (2 g, 2.53 mmol, 1.0 eq) and 4 Å MS (2 g) in DMF (20 mL) was stirred at 40° C. for 5 hours. Oxone (4.67 g, 7.59 mmol, 3.0 eq) was then added, and the reaction mixture was stirred at 40° C. for 16 hours. The reaction mixture was diluted with ethyl acetate (100 mL) and filtered. The filtrate was washed with aqueous NaHCO₃ (20 mL), water (5×30 mL), brine (30 mL), dried over anhydrous sodium sulfate, filtered, and concentrated in vacuum to give tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate (1.8 g, 1.95 mmol, crude) as a yellow solid. LC/MS (ESI) m/z: 822.2 [M+H]⁺.

Step 2: Preparation of tert-butyl 3-[[2-[[(3S,8R)-3-[[tert-butyl(diphenyl)silyl]oxymethyl]-1,2,3,5,6,7-hexahydropyrrolizin-8-yl]methoxy]-8-fluoro-7-[7-fluoro-3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate

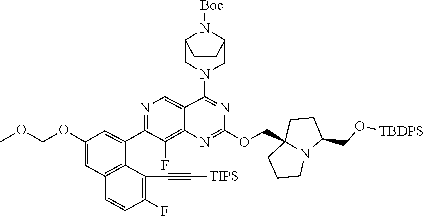

To a solution of tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate (500 mg, 0.608 mmol, 1.0 eq), 4 Å MS (500 mg), and [(3S,8R)-3-[[tert-butyl(diphenyl)silyl]oxymethyl]-1,2,3,5,6,7-hexahydropyrrolizin-8-yl]methanol (249 mg, 0.608 mmol, 1.0 eq) in THF (6 mL) at 0° C. was added t-BuONa (146 mg, 1.52 mmol, 2.5 eq) under N₂, and the reaction mixture was stirred at 0° C. for 28 minutes. The mixture was added to aqueous HCl (0.2 M, 8 mL) to pH=8 then extracted with dichloromethane (3×40 mL). The combined organic extract was washed with brine (20 mL), dried over anhydrous sodium sulfate, filtered, and concentrated. The resulting residue was purified by flash chromatography on SiO₂ (gradient: 0˜20% EtOAc in petroleum ether) to afford tert-butyl 3-[[2-[[(3S,8R)-3-[[tert-butyl(diphenyl)silyl]oxymethyl]-1,2,3,5,6,7-hexahydropyrrolizin-8-yl]methoxy]-8-fluoro-7-[7-fluoro-3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (294 mg, 0.240 mmol, 39% yield) as a yellow oil. LC/MS (ESI) m/z: 576.5 [M/2+H]⁺.

Step 3: Preparation of tert-butyl 3-[7-[8-ethynyl-7-fluoro-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

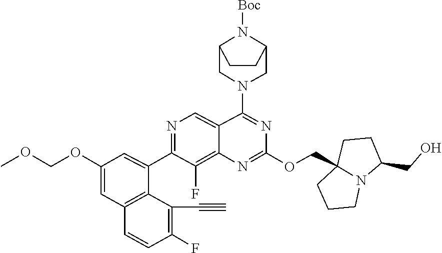 [SMILES: C#Cc1c(F)ccc2cc(OCOC)cc(-c3ncc4c(N5CC6CCC(C5)N6C(=O)OC(C)(C)C)nc(OC[C@]56CCCN5[C@H](CO)CC6)nc4c3F)c12]

To a solution of tert-butyl 3-[[2-[[(3S,8R)-3-[[tert-butyl(diphenyl)silyl]oxymethyl]-1,2,3,5,6,7-hexahydropyrrolizin-8-yl]methoxy]-8-fluoro-7-[7-fluoro-3-(methoxymethoxy)-8-(2-triisopropylsilylethynyl)-1-naphthyl]pyrido[4,3-d]pyrimidin-4-yl]-3,8-diazabicyclo[3.2.1]octane-8-carboxylate (294 mg, 0.255 mmol, 1.0 eq) in DMF (4 mL) at 30° C. was added CsF (310 mg, 2.04 mmol, 8.0 eq), and the reaction mixture was stirred at 30° C. for 12 hours. Water (20 mL) was then added, and the aqueous phase was extracted with EtOAc (3×30 mL). The combined organic extract was washed with brine (20 mL), dried with anhydrous Na₂SO₄, filtered, and concentrated in vacuum. The residue was purified by flash chromatography on SiO₂ (gradient: 0˜7% CH₃OH in CH₂Cl₂) to afford tert-butyl 3-[7-[8-ethynyl-7-fluoro-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (167 mg, 0.199 mmol, 78% yield) as a yellow solid. LC/MS (ESI) m/z: 757.3 [M+H]⁺.

Step 4: Preparation of tert-butyl 3-[7-[8-ethynyl-7-fluoro-3-(methoxymethoxy)-1-naphthyl]-8-fluoro-2-[[(3S,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

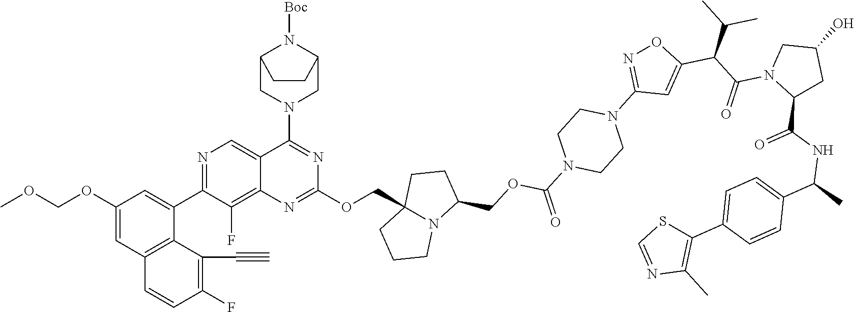 [SMILES: C#Cc1c(F)ccc2cc(OCOC)cc(-c3ncc4c(N5CC6CCC(C5)N6C(=O)OC(C)(C)C)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To tert-butyl 3-[7-[8-ethynyl-7-fluoro-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (167 mg, 0.221 mmol, 1.0 eq) in THF (6 mL) were added triethylamine (1.77 mmol, 246 μL, 8.0 eq), DMAP (8 mg, 0.066 mmol, 0.3 eq), and (4-nitrophenyl) carbonochloridate (85 mg, 0.419 mmol, 1.9 eq), and the reaction mixture was stirred at 28° C. for 15 hours. Triethylamine (0.662 mmol, 92 μL, 3.0 eq) and (2S,4R)-4-hydroxy-1-[(2R)-3-methyl-2-(3-piperazin-1-ylisoxazol-5-yl) butanoyl]-N-[(1S)-1-[4-(4-methylthiazol-5-yl)phenyl]ethyl]pyrrolidine-2-carboxamide (250 mg, 0.441 mmol, 2.0 eq) were then added, and the reaction mixture was stirred at 28° C. for 1 hour. The reaction mixture was filtered, and the solvent concentrated under reduced pressure. The residue was purified by flash chromatography on SiO₂ (gradient: 0˜5% CH₃OH (1N NH₃) in CH₂Cl₂) to afford tert-butyl 3-[7-[8-ethynyl-7-fluoro-3-(methoxymethoxy)-1-naphthyl]-8-fluoro-2-[[(3S,8R)-3-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (190 mg, 0.132 mmol, 60% yield) as a yellow solid. LC/MS (ESI) m/z: 1349.7 [M+H]⁺.

Step 5: Preparation of [(3S,7aR)-7a-{[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

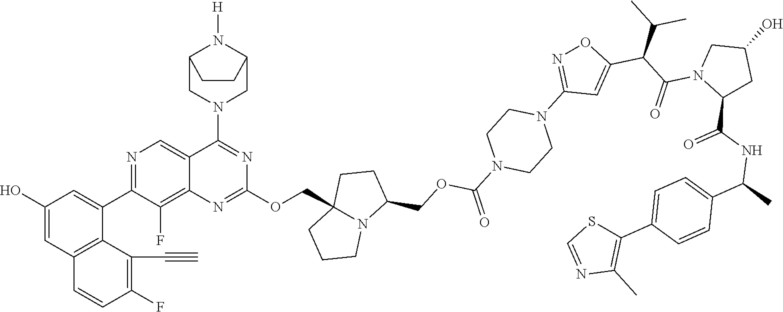 [SMILES: C#Cc1c(F)ccc2cc(O)cc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To tert-butyl 3-[7-[8-ethynyl-7-fluoro-3-(methoxymethoxy)-1-naphthyl]-8-fluoro-2-[[(3S,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (125 mg, 0.093 mmol, 1.0 eq) in dichloromethane (2 mL) was added HCl/dioxane (4 M, 23 μL, 1.0 eq) in one portion at 20° C., and the reaction mixture was stirred at 20° C. for 30 minutes. Petroleum ether (25 mL) was then added, and the resulting mixture was filtered. The cake was dissolved EtOAc (20 mL) and THF (4 mL), then adjusted to pH=8 by addition of saturated aqueous NaHCO₃. The resulting mixture was extracted with EtOAc (3×30 mL), and the combined organic extract was washed with brine (30 mL), dried over anhydrous Na₂SO₄, filtered, and concentrated in vacuum. The residue was purified by prep-HPLC (column: DAICEL CHIRALPAK IA (250 mm*30 mm, 10 um); mobile phase: [45% CH₃CN in EtOH (0.1% NH₄OH)]). The pure fractions were combined and concentrated under reduced pressure, then washed with n-hexane (3×4 mL), dissolved in water and CH₃CN, then lyophilized to afford [(3S,7aR)-7a-{[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (101.1 mg, 0.080 mmol, 61% yield) as a yellow solid. LC/MS (ESI) m/z: 1206.0 [M+H]⁺. ¹H NMR (400 MHz, CD₃OD) δ 9.09-8.98 (m, 1H), 8.91-8.82 (m, 1H), 7.89-7.80 (m, 1H), 7.45-7.31 (m, 6H), 7.23 (d, J=2.0 Hz, 1H), 6.14-6.04 (m, 1H), 5.06-5.00 (m, 1H), 4.65-4.58 (m, 3H), 4.55-4.50 (m, 1H), 4.43 (s, 1H), 4.30-4.19 (m, 2H), 4.15-4.07 (m, 1H), 4.01 (dd, J=6.8, 10.8 Hz, 1H), 3.83 (dd, J=4.0, 10.4 Hz, 1H), 3.72-3.63 (m, 4H), 3.65-3.54 (m, 6H), 3.23 (d, J=2.8 Hz, 4H), 3.10-3.02 (m, 2H), 2.87-2.79 (m, 1H), 2.49-2.46 (m, 3H), 2.41-2.32 (m, 1H), 2.25-2.14 (m, 2H), 2.06-1.99 (m, 1H), 1.95-1.76 (m, 10H), 1.73-1.64 (m, 1H), 1.51 (dd, J=4.8, 7.2 Hz, 3H), 1.05 (d, J=6.4 Hz, 3H), 0.91-0.85 (m, 3H).

Exemplary Synthesis of [(3S,7aR)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 57)

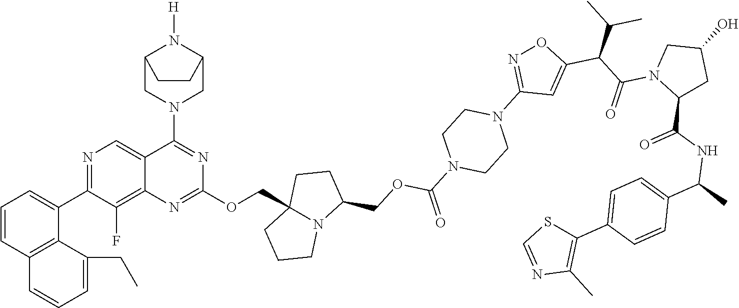 [SMILES: CCc1cccc2cccc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 53 starting from [(3S,8R)-3-[[tert-butyl(diphenyl)silyl]oxymethyl]-1,2,3,5,6,7-hexahydropyrrolizin-8-yl]methanol and tert-butyl 3-[7-(8-ethyl-1-naphthyl)-8-fluoro-2-methylsulfonyl-pyrido[4,3-d]pyrimidin-4-yl]-3,8-diazabicyclo[3.2.1]octane-8-carboxylate, and purified by prep-HPLC (column: Phenomenex C18 75*30 mm*3 um; mobile phase: [20-60% CH₃CN in water (formic acid)]). The pure fractions were combined and concentrated under reduced pressure, then lyophilized to afford [(3S,7aR)-7a-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (193.4 mg, 158.16 umol, 61% yield, formic acid salt) as a white solid. LC/MS (ESI) m/z: 1176.0 [M+H]⁺. ¹H NMR (400 MHz, DMSO-d₆) δ 9.12 (s, 1H), 8.98 (s, 1H), 8.42 (br d, J=7.6 Hz, 1H), 8.08 (d, J=8.0 Hz, 1H), 7.91 (d, J=8.0 Hz, 1H), 7.58 (dt, J=4.8, 7.6 Hz, 1H), 7.51 (t, J=7.6 Hz, 1H), 7.47-7.41 (m, 3H), 7.40-7.33 (m, 3H), 6.17 (s, 1H), 4.91 (quin, J=7.2 Hz, 1H), 4.61-4.41 (m, 3H), 4.36 (br t, J=7.6 Hz, 1H), 4.28 (br s, 1H), 4.15-4.10 (m, 1H), 4.05 (br d, J=10.4 Hz, 1H), 3.98-3.83 (m, 5H), 3.82-3.76 (m, 1H), 3.71 (br dd, J=3.6, 10.4 Hz, 2H), 3.59 (br d, J=9.6 Hz, 1H), 3.49-3.39 (m, 5H), 3.15 (br s, 4H), 2.97-2.90 (m, 2H), 2.70-2.63 (m, 1H), 2.45 (s, 3H), 2.39-2.18 (m, 3H), 2.07-1.90 (m, 3H), 1.87-1.73 (m, 8H), 1.71-1.50 (m, 3H), 1.45-1.34 (m, 3H), 1.00-0.91 (m, 3H), 0.87-0.75 (m, 6H).

Exemplary Synthesis of [(3S,7aS)-7a-{[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 58)

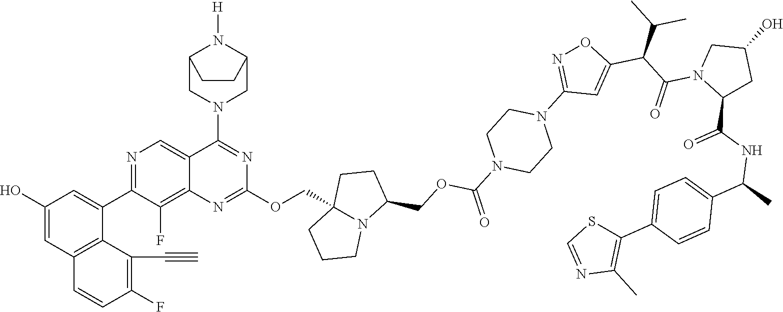 [SMILES: C#Cc1c(F)ccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 56 starting from tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate and [(3S,8S)-3-[[tert-butyl(diphenyl)silyl]oxymethyl]-1,2,3,5,6,7-hexahydropyrrolizin-8-yl]methanol, and purified by prep-HPLC (column: DAICEL CHIRALPAK IC (250 mm*30 mm, 10 um); mobile phase: [40% CH₃CN in EtOH (0.1% NH₄OH)]). The pure fractions were combined and concentrated under reduced pressure, then lyophilized to afford [(3S,7aS)-7a-{[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (175.4 mg, 144.13 umol, 64% yield) as an off-white solid. LC/MS (ESI) m/z: 1205.9 [M+H]⁺. ¹H NMR (400 MHz, DMSO-d₆) δ 9.03 (s, 1H), 8.98 (s, 1H), 8.40 (d, J=7.6 Hz, 1H), 7.96 (dd, J=6.0, 9.2 Hz, 1H), 7.50-7.40 (m, 3H), 7.40-7.32 (m, 3H), 7.17 (d, J=2.4 Hz, 1H), 6.16 (s, 1H), 4.91 (quin, J=7.2 Hz, 1H), 4.46 (br d, J=11.2 Hz, 1H), 4.40-4.23 (m, 4H), 4.21-4.08 (m, 2H), 4.03 (br d, J=10.4 Hz, 1H), 3.93 (s, 1H), 3.74-3.67 (m, 1H), 3.66-3.50 (m, 6H), 3.44 (br d, J=8.8 Hz, 7H), 3.17 (br s, 4H), 2.77-2.66 (m, 2H), 2.45 (s, 3H), 2.26-2.16 (m, 1H), 2.07-1.98 (m, 2H), 1.82-1.61 (m, 12H), 1.57-1.40 (m, 2H), 1.37 (d, J=7.2 Hz, 2H), 0.99-0.91 (m, 3H), 0.83-0.76 (m, 3H).

Exemplary Synthesis of [(2S,5S)-5-{[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]methyl}-1-methylpyrrolidin-2-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 59)

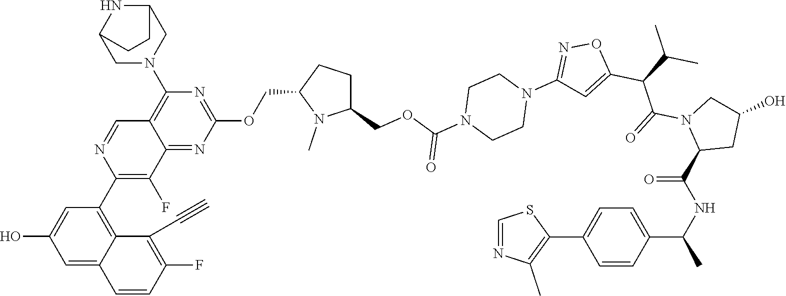 [SMILES: C#Cc1c(F)ccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12]

The title compound was prepared in an analogous manner to Compound 50 starting from tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate and [(2S,5S)-5-(hydroxymethyl)-1-methyl-pyrrolidin-2-yl]methyl benzoate, and purified by preparative HPLC (Column: Daicel Chiralpak IA (250 mm*30 mm, 10 um); Mobile phase: [40% CH₃CN in EtOH (0.1% NH₄O) H]) to afford [(2S,5S)-5-{[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]methyl}-1-methylpyrrolidin-2-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate as an off-white solid. LC/MS (ESI) m/z: 1179.9 [M+H]⁺. ¹H NMR (400 MHz, DMSO-d₆) δ 10.28-10.07 (m, 1H), 9.03 (s, 1H), 8.98 (s, 1H), 8.40 (d, J=7.6 Hz, 1H), 7.97 (dd, J=9.2, 6.0, Hz, 1H), 7.55-7.29 (m, 6H), 7.17 (d, J=2.4 Hz, 1H), 6.21-6.02 (m, 1H), 5.11 (d, J=2.8 Hz, 1H), 4.96-4.86 (m, 1H), 4.55-4.14 (m, 6H), 4.12-3.96 (m, 2H), 3.93 (s, 1H), 3.71 (dd, J=10.4, 4.4 Hz, 1H), 3.66-3.41 (m, 9H), 3.30-3.10 (m, 9H), 2.45 (s, 3H), 2.25-2.15 (m, 1H), 2.05-1.94 (m, 3H), 1.82-1.74 (m, 1H), 1.72-1.55 (m, 6H), 1.47-1.35 (m, 3H), 1.23 (s, 2H), 1.00-0.90 (m, 3H), 0.83-0.74 (m, 3H).

Exemplary Synthesis of (2S,4R)-1-[(2R)-2-{3-[(1-{2-[(4-{3,8-diazabicyclo[3.2.1]octan-3-yl}-8-fluoro-7-(3-hydroxy-8-methylnaphthalen-1-yl)pyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 60)

Step 1: Preparation of 6-methoxytetralin-1-one oxime

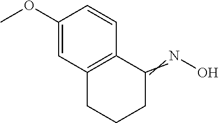

To 6-methoxytetralin-1-one (5.0 g, 28.38 mmol, 1.0 eq) in ethanol (57 mL) and pyridine (5.7 mL) was added NH₂OH·HCl (3.9 g, 56.75 mmol, 2.0 eq), and the reaction mixture was stirred at 25° C. for 16 hours. The solution was concentrated in vacuum. The mixture was poured into water (50 mL) and stirred for 10 minutes. The mixture was filtered through celite pad, and the filtrate was diluted with dichloromethane (60 mL), dried over anhydrous sodium sulfate, filtered, and concentrated to afford 6-methoxytetralin-1-one oxime (5.2 g, 27.28 mmol, 96% yield) as a light yellow solid confirmed. LC/MS (ESI) m/z: 192.3 [M+H]⁺. ¹H NMR (400 MHz, CDCl₃) δ 7.85 (d, J=8.8 Hz, 1H), 6.80-6.77 (m, 1H), 6.67 (d, J=8.8 Hz, 1H), 3.83 (s, 3H), 2.82 (t, J=6.4 Hz, 2H), 2.75 (t, J=6.0 Hz, 2H), 1.92-1.85 (m, 2H).

Step 2: Preparation of 8-bromo-6-methoxy-tetralin-1-one oxime

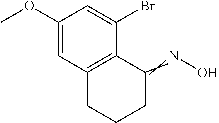

To a solution of 6-methoxytetralin-1-one oxime (3.4 g, 17.78 mmol, 1.0 eq) in acetic acid (65 mL) were added Pd(OAc)₂ (798 mg, 3.56 mmol, 0.2 eq) and N-bromosuccinimide (3.8 g, 21.34 mmol, 1.2 eq), and the reaction mixture was stirred at 40° C. for 1 hour under N₂. The reaction mixture was filtered through celite and concentrated. The residue was dissolved in methyl tert-butyl ether (100 mL), and the resulting solution was washed with water (100 mL), 1 M NaOH solution (100 mL×3), brine (100 mL), and concentrated to afford 8-bromo-6-methoxy-tetralin-1-one oxime (2.1 g, crude) as a brown solid. LC/MS (ESI) m/z: 270.1 [M+H]⁺.

Step 3: Preparation of 8-bromo-6-methoxy-tetralin-1-one

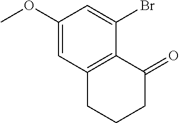

To a solution of 8-bromo-6-methoxy-tetralin-1-one oxime (2.1 g, 7.66 mmol, 1.0 eq) in 1,4-dioxane (20 mL) was added hydrochloric acid (6 M, 32 mL, 25 eq), and the reaction mixture was stirred at 110° C. for 1 hour. The reaction mixture was extracted with methyl tert-butyl ether (3×80 mL). The combined organic extract was washed with 1 M NaOH solution (100 mL) and brine (100 mL), dried over anhydrous Na₂SO₄ and concentrated under reduced pressure. The resulting residue was purified by prep-HPLC (column: Phenomenex luna C18 250*50 mm*10 um; mobile phase: [20-60% CH₃CN in water (formic acid)]). The pure fractions were combined and dried by lyophilization to afford 8-bromo-6-methoxy-tetralin-1-one (1.2 g, 4.42 mmol, 57% yield) as a brown solid. LC/MS (ESI) m/z: 254.9 [M+H]⁺. ¹H NMR (400 MHz, CDCl₃) δ 7.12 (d, J=2.4 Hz, 1H), 6.71 (d, J=2.4 Hz, 1H), 3.85 (s, 3H), 2.95 (t, J=6.0 Hz, 2H), 2.66 (t, J=6.4 Hz, 2H), 2.09-2.06 (m, 2H).

Step 4: Preparation of 8-bromo-6-methoxy-1-methyl-tetralin-1-ol

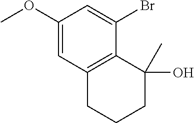

To a solution of 8-bromo-6-methoxy-tetralin-1-one (590 mg, 2.31 mmol, 1.0 eq) in toluene (8 mL) at 0° C. was added trimethylaluminum (2 M, 5.7 mL, 5.0 eq), and the reaction mixture was allowed to warm to 20° C. and stirred for 16 hours. The reaction mixture was poured onto ice cold saturated aqueous NaHCO₃ (15 ml) and extracted with ethyl acetate (3×20 mL). The combined organic extracts were dried over Na₂SO₄, filtered, and concentrated under reduced pressure. The crude product was purified by flash chromatography on SiO₂ (gradient: 0˜7% EtOAc in petroleum ether) to afford 8-bromo-6-methoxy-1-methyl-tetralin-1-ol (482 mg, 1.42 mmol, 61% yield) as a colorless oil. LC/MS (ESI) m/z: 254.9 [M−H₂O+H]⁺. ¹H NMR (400 MHz, CDCl₃) δ 7.00 (d, J=2.4 Hz, 1H), 6.60 (d, J=2.4 Hz, 1H), 3.77 (s, 3H), 2.87-2.78 (m, 2H), 1.99-1.96 (m, 2H), 1.90-1.86 (m, 1H), 1.80-1.76 (m, 1H), 1.74 (s, 3H).

Step 5: Preparation of 5-bromo-7-methoxy-4-methyl-1,2-dihydronaphthalene

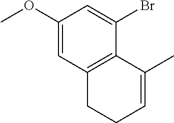

To a solution of 8-bromo-6-methoxy-1-methyl-tetralin-1-ol (482 mg, 1.78 mmol, 1.0 eq) in dichloromethane (6 mL) was added 4-methylbenzenesulfonic acid hydrate (17 mg, 0.09 mmol, 0.05 eq), and the reaction mixture was stirred at 20° C. for 1 hour. The reaction mixture was poured into water (4 ml) and extracted with EtOAc (3×20 mL). The combined organic extracts were dried over Na₂SO₄, filtered, and concentrated under reduced pressure. The crude product was purified by flash chromatography on SiO₂ (gradient: 0˜1% EtOAc in petroleum ether) to afford 5-bromo-7-methoxy-4-methyl-1,2-dihydronaphthalene (322 mg, 1.02 mmol, 57% yield) as colorless oil. LC/MS (ESI) m/z: 254.8 [M+H]⁺. ¹H NMR (400 MHz, CDCl₃) δ 7.00 (d, J=2.8 Hz, 1H), 6.70 (d, J=2.8 Hz, 1H), 6.00-5.97 (m, 1H), 3.79 (s, 3H), 2.66-2.62 (m, 2H), 2.34 (d, J=1.2 Hz, 3H), 2.09-2.04 (m, 2H).

Step 6: Preparation of 1-bromo-3-methoxy-8-methyl-naphthalene

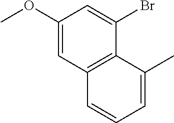

To a solution of 5-bromo-7-methoxy-4-methyl-1,2-dihydronaphthalene (322 mg, 1.27 mmol, 1.0 eq) in dichloromethane (4 mL) was added 2,3-dichloro-5,6-dicyano-1,4-benzoquinone (433 mg, 1.91 mmol, 1.5 eq), and the reaction mixture was stirred at 20° C. for 2 hours. The mixture was poured into saturated aqueous NaHCO₃ (5 mL). The mixture was filtered, and the filtrate was extracted with dichloromethane (3×20 mL). The combined organic extracts were washed with brine (60 mL), dried over Na₂SO₄, filtered, and concentrated under reduced pressure. The crude product was purified by flash chromatography on SiO₂ (gradient: 0˜2% EtOAc in petroleum ether) to afford 1-bromo-3-methoxy-8-methyl-naphthalene (247 mg, 0.98 mmol, 77% yield) as a white solid. LC/MS (ESI) m/z: 250.9 [M+H]⁺. ¹H NMR (400 MHz, CDCl₃) δ 7.60 (d, J=8.0 Hz, 1H), 7.55 (d, J=2.4 Hz, 1H), 7.32-7.30 (m, 1H), 7.20 (d, J=7.2 Hz, 1H), 7.10 (d, J=2.4 Hz, 1H), 3.90 (s, 3H), 3.09 (s, 3H).

Step 7: Preparation of 4-bromo-5-methyl-naphthalen-2-ol

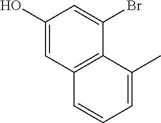

To a solution of 1-bromo-3-methoxy-8-methyl-naphthalene (391 mg, 1.56 mmol, 1.0 eq) in dichloromethane (5 mL) at −78° C. was added BBr₃ (1.87 mmol, 0.18 mL, 1.2 eq), and the reaction mixture was warmed to 20° C. and stirred for 4 hours. The mixture was poured into water (5 ml) and extracted with dichloromethane (3×15 mL). The combined organic extracts were washed with brine (45 mL), dried over Na₂SO₄, filtered, and concentrated under reduced pressure. The crude product was purified by flash chromatography on SiO₂ (gradient: 0˜5% EtOAc in petroleum ether) to afford 4-bromo-5-methyl-naphthalen-2-ol (365 mg, 1.37 mmol, 88% yield) as colorless oil. LC/MS (ESI) m/z: 239.1 [M+H]⁺. ¹H NMR (400 MHz, CDCl₃) δ 7.56-7.50 (m, 2H), 7.30-7.29 (m, 1H), 7.20 (d, J=6.4 Hz, 1H), 7.13 (d, J=2.8 Hz, 1H), 4.88 (s, 1H), 3.09 (s, 3H).

Step 8: Preparation of 1-bromo-3-(methoxymethoxy)-8-methyl-naphthalene

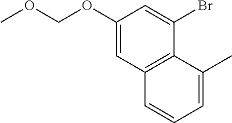

To a solution of 4-bromo-5-methyl-naphthalen-2-ol (365 mg, 1.54 mmol, 1.0 eq) and N-ethyl-N-isopropylpropan-2-amine (4.62 mmol, 0.8 mL, 3.0 eq) in dichloromethane (5 mL) at 0° C. was added MOMCl (1.24 mmol, 0.09 mL, 0.8 eq) under N₂, and the reaction mixture was stirred at 0° C. for 1 hour. The reaction was quenched by addition of H₂O (5 mL), and the aqueous phase was extracted with dichloromethane (3×30 mL). The combined organic extracts were washed with brine (50 mL), dried over Na₂SO₄, filtered, and concentrated. The crude product was purified by flash chromatography on SiO₂ (gradient: 0˜1% EtOAc in petroleum ether) to afford 1-bromo-3-(methoxymethoxy)-8-methyl-naphthalene (360 mg, 1.20 mmol, 78% yield) as colorless oil. LC/MS (ESI) m/z: 281.0 [M+H]⁺. ¹H NMR (400 MHz, CDCl₃) δ 7.62-7.60 (m, 2H), 7.36 (d, J=2.8 Hz, 1H), 7.32-7.30 (m, 1H), 7.22 (d, J=7.2 Hz, 1H), 5.27 (s, 2H), 3.52 (s, 3H), 3.09 (s, 3H).

Step 9: Preparation of 2-[3-(methoxymethoxy)-8-methyl-1-naphthyl]-4,4,5,5-tetramethyl-1,3,2-dioxaborolane

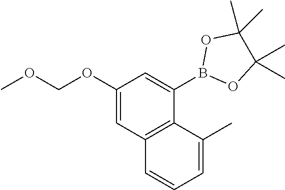

To a solution of 1-bromo-3-(methoxymethoxy)-8-methyl-naphthalene (310 mg, 1.10 mmol, 1.0 eq) and 4,4,5,5-tetramethyl-2-(4,4,5,5-tetramethyl-1,3,2-dioxaborolan-2-yl)-1,3,2-dioxaborolane (700 mg, 2.76 mmol, 2.5 eq) in dioxane (5 mL) were added potassium acetate (325 mg, 3.31 mmol, 3.0 eq) and Pd(dppf) Cl₂ (121 mg, 0.16 mmol, 0.15 eq), and the reaction mixture was stirred at 95° C. for 15 hours under N₂. The mixture was diluted with EtOAc (30 mL), then filtered and concentrated. The crude product was purified by flash chromatography on SiO₂ (gradient: 0% EtOAc in petroleum ether) to afford 2-[3-(methoxymethoxy)-8-methyl-1-naphthyl]-4,4,5,5-tetramethyl-1,3,2-dioxaborolane (151 mg, 0.37 mmol, 33% yield) as a light green oil. LC/MS (ESI) m/z: 329.2 [M+H]⁺. ¹H NMR (400 MHz, CDCl₃) δ 7.63-7.56 (m, 2H), 7.41 (d, J=2.8 Hz, 1H), 7.36 (d, J=2.8 Hz, 1H), 7.20 (d, J=6.4 Hz, 1H), 5.30 (s, 2H), 3.52 (s, 3H), 2.77 (s, 3H), 1.27 (s, 12H).

Step 10: Preparation of tert-butyl 3-[[2-(2,2-dimethoxyethoxy)-8-fluoro-7-[3-(methoxymethoxy)-8-methyl-1-naphthyl]pyrido[4,3-d]pyrimidin-4-yl]-3,8-diazabicyclo[3.2.1]octane-8-carboxylate

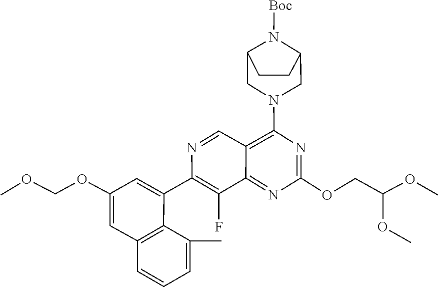

To a solution of tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (190 mg, 0.38 mmol, 1.0 eq) and 2-[3-(methoxymethoxy)-8-methyl-1-naphthyl]-4,4,5,5-tetramethyl-1,3,2-dioxaborolane (150 mg, 0.46 mmol, 1.2 eq) in dioxane (2 mL) and H₂O (0.4 mL) were added potassium phosphate (243 mg, 1.14 mmol, 3.0 eq) and CATACXIUM® A PD G3 (56 mg, 0.08 mmol, 0.2 eq), and the reaction mixture was stirred at 90° C. for 16 hours under N₂. The reaction mixture was diluted with EtOAc (30 mL), then washed with water (3×20 mL) and brine (20 mL), dried over Na₂SO₄, filtered, and concentrated. The crude product was purified by flash chromatography on SiO₂ (gradient: 0˜33% EtOAc in petroleum ether) to afford tert-butyl 3-[2-(2,2-dimethoxyethoxy)-8-fluoro-7-[3-(methoxymethoxy)-8-methyl-1-naphthyl]pyrido[4,3-d]pyrimidin-4-yl]-3,8-diazabicyclo[3.2.1]octane-8-carboxylate (138 mg, 0.19 mmol, 50% yield) as light yellow oil. LC/MS (ESI) m/z: 664.8 [M+H]⁺.

Step 11: Preparation of tert-butyl 3-[8-fluoro-7-(3-hydroxy-8-methyl-1-naphthyl)-2-(2-oxoethoxy)pyrido[4,3-d]pyrimidin-4-yl]-3,8-diazabicyclo[3.2.1]octane-8-carboxylate

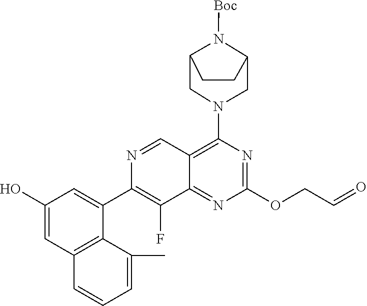 [SMILES: Cc1cccc2cc(O)cc(-c3ncc4c(N5CC6CCC(C5)N6C(=O)OC(C)(C)C)nc(OCC=O)nc4c3F)c12]

To a solution of tert-butyl 3-[[2-(2,2-dimethoxyethoxy)-8-fluoro-7-[3-(methoxymethoxy)-8-methyl-1-naphthyl]pyrido[4,3-d]pyrimidin-4-yl]-3,8-diazabicyclo[3.2.1]octane-8-carboxylate (118 mg, 0.18 mmol, 1.0 eq) in acetone (0.4 mL) was added hydrochloric acid (12 M, 0.4 mL, 30 eq), and the reaction mixture was stirred at 20° C. for 6 minutes, then poured onto a mixture of Boc₂O (0.23 mmol, 53.10 uL, 1.3 eq) and NaHCO₃ (747 mg, 8.89 mmol, 50 eq) in tetrahydrofuran (3 mL) and H₂O (3 mL). The resulting mixture was stirred at 20° C. for 30 minutes. The mixtures was poured onto water (5 mL), and the aqueous phase was extracted with EtOAc (3×15 mL). The combined organic extracts were washed with brine (20 mL), dried over Na₂SO₄, filtered, and concentrated. The crude product was purified by flash chromatography on SiO₂ (gradient: 0˜45% EtOAc in petroleum ether) to afford tert-butyl 3-[8-fluoro-7-(3-hydroxy-8-methyl-1-naphthyl)-2-(2-oxoethoxy)pyrido[4,3-d]pyrimidin-4-yl]-3,8-diazabicyclo[3.2.1]octane-8-carboxylate (54 mg, 0.09 mmol, 48% yield) as a white solid. LC/MS (ESI) m/z: 574.3 [M+H]⁺.

Step 12: Preparation of (2S,4R)-1-[(2R)-2-{3-[(1-{2-[(4-{3,8-diazabicyclo[3.2.1]octan-3-yl}-8-fluoro-7-(3-hydroxy-8-methylnaphthalen-1-yl)pyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

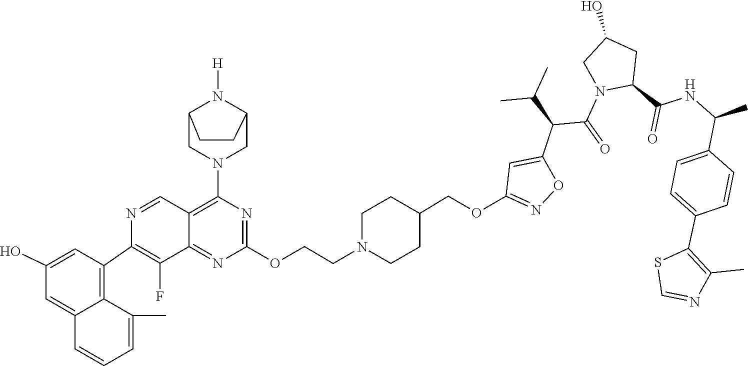 [SMILES: Cc1ncsc1-c1ccc([C@H](C)NC(=O)[C@@H]2C[C@@H](O)CN2C(=O)[C@@H](c2cc(OCC3CCN(CCOc4nc(N5CC6CCC(C5)N6)c5cnc(-c6cc(O)cc7cccc(C)c67)c(F)c5n4)CC3)no2)C(C)C)cc1]

The title compound was made in an analogous manner to Compound 22 and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide, and purified by prep-HPLC (column: Phenomenex C18 75*30 mm*3 um; mobile phase: [20-60% CH₃CN in water (formic acid)]). The pure fractions were combined and dried by lyophilization to afford (2S,4R)-1-[(2R)-2-{3-[(1-{2-[(4-{3,8-diazabicyclo[3.2.1]octan-3-yl}-8-fluoro-7-(3-hydroxy-8-methylnaphthalen-1-yl)pyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (formic acid salt) as a white solid. LC/MS (ESI) m/z: 1053.3 [M+H]⁺. ¹H NMR (400 MHz, CD₃OD) δ 9.09 (s, 1H), 8.88 (s, 1H), 7.64 (d, J=8.4 Hz, 1H), 7.44-7.29 (m, 6H), 7.07 (d, J=6.4 Hz, 1H), 7.02 (d, J=2.4 Hz, 1H), 5.99 (s, 1H), 5.01-4.99 (m, 1H), 4.78-4.70 (m, 5H), 4.52-4.44 (m, 2H), 4.10-4.08 (m, 2H), 3.98 (s, 2H), 3.88-3.80 (m, 3H), 3.69-3.59 (m, 2H), 3.51-3.46 (m, 3H), 2.78-2.75 (m, 2H), 2.48 (s, 3H), 2.39-2.34 (m, 1H), 2.21-2.16 (m, 1H), 1.98-1.96 (m, 11H), 1.59-1.58 (m, 2H), 1.53-1.51 (m, 3H), 1.05 (d, J=6.4 Hz, 3H), 0.89-0.87 (m, 3H).

Exemplary Synthesis of (2S,4R)-1-[(2R)-2-(3-{[(3S,7aS)-7a-{[(4-{3,8-diazabicyclo[3.2.1]octan-3-yl}-7-(7,8-difluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 61)

Step 1: Preparation of methyl 2-[3-[[(3S,8S)-8-[[tert-butyl(diphenyl)silyl]oxymethyl]-1,2,3,5,6,7-hexahydropyrrolizin-3-yl]methoxy]isoxazol-5-yl]-3-methyl-butanoate

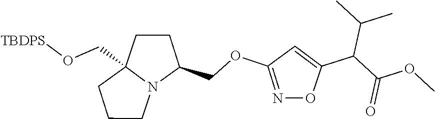

To a mixture of [(3S,8S)-8-[[tert-butyl(diphenyl)silyl]oxymethyl]-1,2,3,5,6,7-hexahydropyrrolizin-3-yl]methanol (5.0 g, 12.21 mmol, 1.0 eq), methyl 2-(3-hydroxyisoxazol-5-yl)-3-methyl-butanoate (2.4 g, 12.21 mmol, 1.0 eq), and PPh₃ (6.4 g, 24.41 mmol, 2.0 eq) in toluene (50 mL) at 25° C. was added DIAD (24.41 mmol, 4.7 mL, 2.0 eq) under N₂, and the reaction mixture was stirred at 110° C. for 15 hours. The mixture was concentrated, and the resulting residue was purified by flash chromatography on SiO₂ (gradient: 0˜10% EtOAc in petroleum ether) to afford methyl 2-[3-[[(3S,8S)-8-[[tert-butyl(diphenyl)silyl]oxymethyl]-1,2,3,5,6,7-hexahydropyrrolizin-3-yl]methoxy]isoxazol-5-yl]-3-methyl-butanoate (5.1 g, 5.27 mmol, 43% yield) as a yellow oil. LC/MS (ESI) m/z: 591.2 [M+H]⁺.

Step 2: Preparation of methyl 2-[3-[[(3S,8S)-8-(hydroxymethyl)-1,2,3,5,6,7-hexahydropyrrolizin-3-yl]methoxy]isoxazol-5-yl]-3-methyl-butanoate

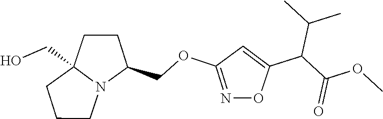 [SMILES: COC(=O)C(c1cc(OC[C@@H]2CC[C@]3(CO)CCCN23)no1)C(C)C]

To a solution of methyl 2-[3-[[(3S,8S)-8-[[tert-butyl(diphenyl)silyl]oxymethyl]-1,2,3,5,6,7-hexahydropyrrolizin-3-yl]methoxy]isoxazol-5-yl]-3-methyl-butanoate (5.1 g, 8.63 mmol, 1.0 eq) in CH₂Cl₂ (50 mL) was added N,N-diethylethanamine trihydrofluoride (129.48 mmol, 21.1 mL, 15.0 eq), and the reaction mixture was stirred at 25° C. for 1 hour. The mixture was concentrated, water (20 mL) was then added. The resulting aqueous mixture was extracted with MTBE (3×20 mL). The pH of the aqueous phase was adjusted to pH ˜8 by addition of NaHCO₃ until. The resulting mixture was extracted with 10:1 CH₂Cl₂:CH₃OH (3×20 mL), and the combined organic extract was washed with brine (3×15 mL), dried over Na₂SO₄, filtered, and concentrated under reduced pressure to afford methyl 2-[3-[[(3S,8S)-8-(hydroxymethyl)-1,2,3,5,6,7-hexahydropyrrolizin-3-yl]methoxy]isoxazol-5-yl]-3-methyl-butanoate (1.4 g, crude) as a yellow oil. LC/MS (ESI) m/z: 353.6 [M+H]⁺.

Step 3: Preparation of 2-[3-[[(3S,8S)-8-(hydroxymethyl)-1,2,3,5,6,7-hexahydropyrrolizin-3-yl]methoxy]isoxazol-5-yl]-3-methyl-butanoic acid

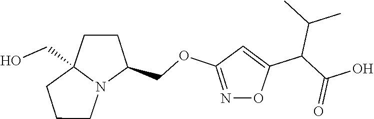 [SMILES: CC(C)C(C(=O)O)c1cc(OC[C@@H]2CC[C@]3(CO)CCCN23)no1]

To a solution of methyl 2-[3-[[(3S,8S)-8-(hydroxymethyl)-1,2,3,5,6,7-hexahydropyrrolizin-3-yl]methoxy]isoxazol-5-yl]-3-methyl-butanoate (1.4 g, 3.97 mmol, 1.0 eq) in THF (15 mL) and H₂O (5 mL) was added LiOH·H₂O (8330 mg, 19.86 mmol, 5.0 eq), and the reaction mixture was stirred at 25° C. for 10 hours. The mixture was diluted with H₂O (20 mL) followed by addition of 2N aqueous HCl until pH ˜4. The resulting mixture was concentrated and lyophilized to afford 2-[3-[[(3S,8S)-8-(hydroxymethyl)-1,2,3,5,6,7-hexahydropyrrolizin-3-yl]methoxy]isoxazol-5-yl]-3-methyl-butanoic acid (1.23 g, crude) as a yellow solid. LC/MS (ESI) m/z: 339.6 [M+H]⁺.

Step 4: Preparation of (2S,4R)-4-[tert-butyl(diphenyl)silyl]oxy-1-[2-[3-[[(3S,8S)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide

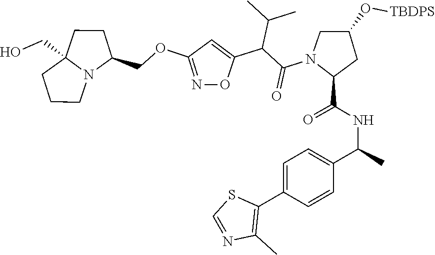

To a solution of (2S,4R)-4-[tert-butyl(diphenyl)silyl]oxy-N-[(1S)-1-[4-(4-methylthiazol-5-yl)phenyl]ethyl]pyrrolidine-2-carboxamide (2.75 g, 3.63 mmol, 80% purity, 1.0 eq, HCl) in CH₂Cl₂ (50 mL) were added DIEA (36.29 mmol, 6.3 mL, 10.0 eq), 2-[3-[[(3S,8S)-8-(hydroxymethyl)-1,2,3,5,6,7-hexahydropyrrolizin-3-yl]methoxy]isoxazol-5-yl]-3-methyl-butanoic acid (1.23 g, 3.63 mmol, 1.0 eq), and HATU (1.52 g, 3.99 mmol, 1.1 eq), and the reaction mixture was stirred at 25° C. for 0.5 hour. The reaction mixture was diluted with water (100 mL) and extracted with CH₂Cl₂ (3×100 mL). The combined organic extract was washed with brine (2×100 mL), dried over Na₂SO₄, filtered, and concentrated. The resulting residue was purified by flash chromatography on SiO₂ (gradient: 0˜10% CH₂Cl₂ in CH₃OH) to afford (2S,4R)-4-[tert-butyl(diphenyl)silyl]oxy-1-[2-[3-[[(3S,8S)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide (1.2 g, 1.35 mmol, 37% yield) as a yellow solid. LC/MS (ESI) m/z: 890.4 [M+H]⁺.

Step 5: Preparation of 3,4-difluoro-11-oxatricyclo[6.2.1.02,7]undeca-2 (7),3,5,9-tetraene

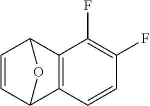 [SMILES: Fc1ccc2c(c1F)C1C=CC2O1]

To a solution of furan (6.5 g, 94.80 mmol, 2.0 eq) and 1-bromo-2,3,4-trifluoro-benzene (10.0 g, 47.40 mmol, 1.0 eq) in toluene (100 mL) at −15° C. was added n-BuLi (2.5 M, 22.75 mL, 1.2 eq), and the reaction mixture was stirred at −15° C. for 0.5 hours, then at 20° C. for 12 hours. The reaction was quenched by addition of H₂O (50 mL), and the aqueous phase was extracted with EtOAc (3×80 mL). The combined organic extract was washed with brine (20 mL), dried with anhydrous Na₂SO₄, filtered, and concentrated. The resulting residue was purified by prep-HPLC (Condition: [40-80% CH₃CN in water (formic acid)]). The pure fractions were combined and concentrated under reduced pressure. The aqueous phase was extracted with ethyl acetate (3×200 mL), and the combined organic extract was washed with brine (50 mL), dried over anhydrous Na₂SO₄, filtered and concentrated to afford 3,4-difluoro-11-oxatricyclo[6.2.1.02,7]undeca-2 (7),3,5,9-tetraene (3.2 g, 17.76 mmol, 37% yield) as a yellow oil. LC/MS (ESI) m/z: 181.0 [M+H]⁺. ¹H NMR (400 MHz, CDCl₃) δ 7.11-7.03 (m, 2H), 6.93 (dd, J=3.6, 7.6 Hz, 1H), 6.76 (td, J=7.2, 10.4 Hz, 1H), 5.98 (s, 1H), 5.72 (s, 1H).

Step 6: Preparation of 7,8-difluoronaphthalen-1-ol

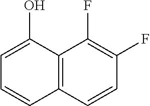 [SMILES: Oc1cccc2ccc(F)c(F)c12]

To a solution of 3,4-difluoro-11-oxatricyclo[6.2.1.02,7]undeca-2 (7),3,5,9-tetraene (3.0 g, 16.65 mmol, 1.0 eq) in EtOH (40 mL) at 20° C. was added HCl (12 M, 20 mL, 14.41 eq), and the reaction mixture was stirred at 80° C. for 2 hours. The mixture was concentrated over vacuum, and the resulting residue was dissolved with EtOAc (40 mL), washed with saturated aqueous NaHCO₃ (3×20 mL), dried over anhydrous Na₂SO₄, filtered, and concentrated. The resulting residue was purified by flash chromatography on SiO₂ (gradient: 0˜3% EtOAc in petroleum ether) to afford 7,8-difluoronaphthalen-1-ol (2.5 g, 13.04 mmol, 73% yield) as a white solid. ¹H NMR (400 MHz, CDCl₃) δ 7.62-7.56 (m, 1H), 7.42-7.36 (m, 2H), 7.36-7.29 (m, 1H), 7.02 (dd, J=1.6, 6.4 Hz, 1H), 6.68-6.60 (m, 1H).

Step 7: Preparation of (7,8-difluoro-1-naphthyl)trifluoromethanesulfonate

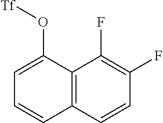

To a solution of 7,8-difluoronaphthalen-1-ol (2.3 g, 12.77 mmol, 1.0 eq) in CH₂Cl₂ (30 mL) at −40° C. were added DIEA (8.25 g, 63.84 mmol, 5.0 eq) and Tf₂O (4.7 g, 16.60 mmol, 1.3 eq), and the reaction mixture was stirred at −40° C. for 30 minutes. The reaction was quenched by addition of H₂O (20 mL), and the aqueous phase was extracted with dichloromethane (3×30 mL). The combined organic extract was washed with brine (20 mL), dried with anhydrous Na₂SO₄, filtered, and concentrated in vacuum. The residue was purified by flash chromatography on SiO₂ (gradient: 0˜2% EtOAc in petroleum ether) to afford (7,8-difluoro-1-naphthyl)trifluoromethanesulfonate (3.8 g, 12.17 mmol, 95% yield) as a yellow oil. ¹H NMR (400 MHz, CDCl₃) δ 7.91-7.85 (m, 1H), 7.70 (ddd, J=1.6, 4.8, 9.2 Hz, 1H), 7.54-7.43 (m, 3H).

Step 8: Preparation of 2-(7,8-difluoro-1-naphthyl)-4,4,5,5-tetramethyl-1,3,2-dioxaborolane

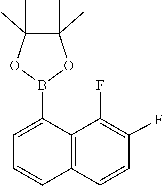 [SMILES: CC1(C)OB(c2cccc3ccc(F)c(F)c23)OC1(C)C]

To a solution of (7,8-difluoro-1-naphthyl)trifluoromethanesulfonate (3.8 g, 12.17 mmol, 1.0 eq) and 4,4,5,5-tetramethyl-2-(4,4,5,5-tetramethyl-1,3,2-dioxaborolan-2-yl)-1,3,2-dioxaborolane (7.7 g, 30.43 mmol, 2.5 eq) in dioxane (50 mL) were added KOAc (3.6 g, 36.51 mmol, 3.0 eq) and Pd(dppf) Cl₂ (890 mg, 1.22 mmol, 0.1 eq), and the reaction mixture was stirred at 100° C. for 12 hours. The reaction mixture was diluted with EtOAc (100 mL), filtered, and concentrated under reduced pressure. The residue was purified by flash chromatography on SiO₂ (gradient: 0˜3% EtOAc in petroleum ether) to afford 2-(7,8-difluoro-1-naphthyl)-4,4,5,5-tetramethyl-1,3,2-dioxaborolane (3.3 g, 11.37 mmol, 93% yield) as a white solid. LC/MS (ESI) m/z: 291.1 [M+H]⁺. ¹H NMR (400 MHz, CDCl₃) δ 7.85 (d, J=8.4 Hz, 1H), 7.72 (d, J=6.8 Hz, 1H), 7.60 (ddd, J=1.6, 4.8, 9.2 Hz, 1H), 7.47 (dd, J=7.2, 8.0 Hz, 1H), 7.34 (dt, J=7.6, 9.6 Hz, 1H), 1.46 (s, 12H).

Step 9: Preparation of tert-butyl 3-[7-(7,8-difluoro-1-naphthyl)-8-fluoro-2-methylsulfanyl-pyrido[4,3-d]pyrimidin-4-yl]-3,8-diazabicyclo[3.2.1]octane-8-carboxylate

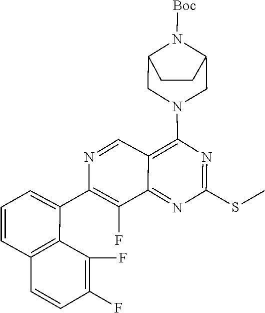 [SMILES: CSc1nc(N2CC3CCC(C2)N3C(=O)OC(C)(C)C)c2cnc(-c3cccc4ccc(F)c(F)c34)c(F)c2n1]

To a solution of 2-(7,8-difluoro-1-naphthyl)-4,4,5,5-tetramethyl-1,3,2-dioxaborolane (1.0 g, 3.45 mmol, 1.2 eq) and tert-butyl 3-(7-chloro-8-fluoro-2-methylsulfanyl-pyrido[4,3-d]pyrimidin-4-yl)-3,8-diazabicyclo[3.2.1]octane-8-carboxylate (1.3 g, 2.87 mmol, 1.0 eq) in dioxane (10 mL) and H₂O (2 mL) were added K₃PO₄ (1.8 g, 8.62 mmol, 3.0 eq) and CataCXium® A PD G3 (209 mg, 0.287 mmol, 0.1 eq), and the reaction mixture was stirred at 80° C. for 12 hours. The mixture was dried with anhydrous Na₂SO₄, filtered, and concentrated in vacuum. The residue was purified by flash chromatography on SiO₂ (gradient: 0˜25% EtOAc in petroleum ether) to afford tert-butyl 3-[7-(7,8-difluoro-1-naphthyl)-8-fluoro-2-methylsulfanyl-pyrido[4,3-d]pyrimidin-4-yl]-3,8-diazabicyclo[3.2.1]octane-8-carboxylate (1.26 g, 2.15 mmol, 75% yield) as a yellow solid. LC/MS (ESI) m/z: 568.1 [M+H]⁺.

Step 10: Preparation f tert-butyl 3-[7-(7,8-difluoro-1-naphthyl)-8-fluoro-2-methylsulfonyl-pyrido[4,3-d]pyrimidin-4-yl]-3,8-diazabicyclo[3.2.1]octane-8-carboxylate

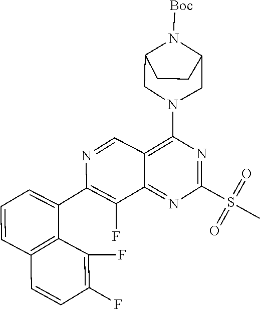 [SMILES: CC(C)(C)OC(=O)N1C2CCC1CN(c1nc(S(C)(=O)=O)nc3c(F)c(-c4cccc5ccc(F)c(F)c45)ncc13)C2]

To a solution of tert-butyl 3-[7-(7,8-difluoro-1-naphthyl)-8-fluoro-2-methylsulfanyl-pyrido[4,3-d]pyrimidin-4-yl]-3,8-diazabicyclo[3.2.1]octane-8-carboxylate (1.26 g, 2.22 mmol, 1.0 eq) in DMF (13 mL) were added 4 Å MS (1.3 g, 2.22 mmol, 1.0 eq), and the resulting mixture was stirred at 20° C. for 12 hours. Oxone (4.1 g, 6.66 mmol, 3.0 eq) was then added, and the reaction mixture was stirred at 20° C. for 18 hours. The reaction was quenched by addition of a solution of Na₂SO₃ (1.4 g, 11.1 mmol, 5.0 eq)/NaHCO₃ (932 mg, 11.1 mmol, 5.0 eq) in H₂O (20 mL) slowly. EtOAc (30 mL) was then added, and the resulting mixture was filtered. The filtrate was separated, and the aqueous layer was extracted with EtOAc (2×20 mL). The combined organic layer was washed with brine (3×20 mL), dried over anhydrous Na₂SO₄, filtered, and concentrated under reduced pressure to afford tert-butyl 3-[7-(7,8-difluoro-1-naphthyl)-8-fluoro-2-methylsulfonyl-pyrido[4,3-d]pyrimidin-4-yl]-3,8-diazabicyclo[3.2.1]octane-8-carboxylate (1.04 g, 1.30 mmol, 59% yield) as a yellow solid. LC/MS (ESI) m/z: 600.1 [M+H]⁺.

Step 11: Preparation of tert-butyl 3-[[2-[[(3S,8S)-3-[[5-[1-[(2S,4R)-4-[tert-butyl(diphenyl)silyl]oxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]-7-(7,8-difluoro-1-naphthyl)-8-fluoro-pyrido[4,3-d]pyrimidin-4-yl]-3,8-diazabicyclo[3.2.1]octane-8-carboxylate

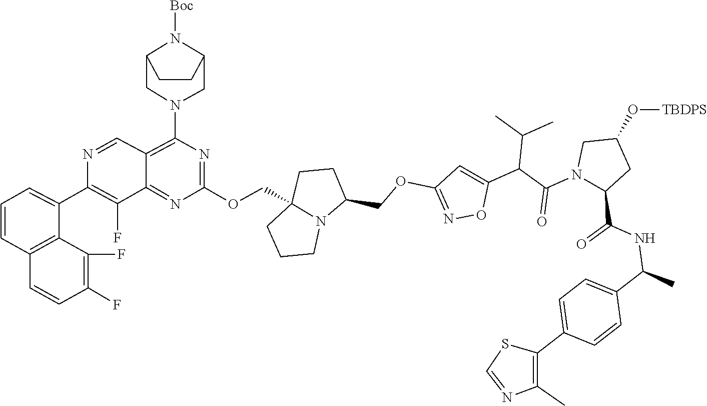

To a solution of (2S,4R)-4-[tert-butyl(diphenyl)silyl]oxy-1-[2-[3-[[(3S,8S)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide (240 mg, 0.270 mmol, 1.0 eq) and tert-butyl 3-[7-(7,8-difluoro-1-naphthyl)-8-fluoro-2-methylsulfonyl-pyrido[4,3-d]pyrimidin-4-yl]-3,8-diazabicyclo[3.2.1]octane-8-carboxylate (259 mg, 75% purity, 0.324 mmol, 1.2 eq) in THF (5 mL) at 0° C. were added 4 Å MS (300 mg, 0.270 mmol, 1.0 eq) and t-BuONa (78 mg, 0.809 mmol, 3.0 eq), and the reaction mixture was stirred at 0° C. for 0.5 hours. The reaction was quenched with HCl (2 M) to pH ˜6 and extracted with dichloromethane (3×30 mL). The organic layers were combined, dried over Na₂SO₄, filtered, and concentrated under reduced pressure. The residue was purified by flash chromatography on SiO₂ (gradient: 0˜5% CH₃OH in dichloromethane) to afford tert-butyl 3-[2-[[(3S,8S)-3-[[5-[1-[(2S,4R)-4-[tert-butyl(diphenyl)silyl]oxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]-7-(7,8-difluoro-1-naphthyl)-8-fluoro-pyrido[4,3-d]pyrimidin-4-yl]-3,8-diazabicyclo[3.2.1]octane-8-carboxylate (310 mg, 0.213 mmol, 79% yield) as a yellow solid. LC/MS (ESI) m/z: 1049.9 [M+H]⁺.

Step 12: Preparation of tert-butyl 3-[7-(7,8-difluoro-1-naphthyl)-8-fluoro-2-[[(3S,8S)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

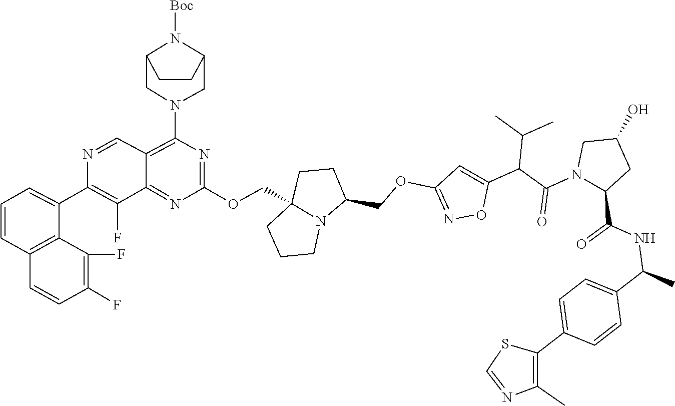 [SMILES: Cc1ncsc1-c1ccc([C@H](C)NC(=O)[C@@H]2C[C@@H](O)CN2C(=O)C(c2cc(OC[C@@H]3CC[C@]4(COc5nc(N6CC7CCC(C6)N7C(=O)OC(C)(C)C)c6cnc(-c7cccc8ccc(F)c(F)c78)c(F)c6n5)CCCN34)no2)C(C)C)cc1]

To a solution of tert-butyl 3-[[2-[[(3S,8S)-3-[[5-[1-[(2S,4R)-4-[tert-butyl(diphenyl)silyl]oxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]-7-(7,8-difluoro-1-naphthyl)-8-fluoro-pyrido[4,3-d]pyrimidin-4-yl]-3,8-diazabicyclo[3.2.1]octane-8-carboxylate (580 mg, 0.411 mmol, 1.0 eq) in DMF (6 mL) was added CsF (500 mg, 3.29 mmol, 8.0 eq), and the reaction mixture was stirred at 30° C. for 12 hours. The reaction was quenched by addition of H₂O (10 mL), then filtered. The filter cake was washed with water (2×10 mL) and dissolved with dichloromethane (3×20 mL). The organic phase was dried with anhydrous Na₂SO₄, filtered, and concentrated. The residue was purified by flash chromatography on SiO₂ (gradient: 0˜10% CH₃OH in dichloromethane) to afford tert-butyl 3-[7-(7,8-difluoro-1-naphthyl)-8-fluoro-2-[[(3S,8S)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (420 mg, 0.319 mmol, 78% yield) as a yellow solid. LC/MS (ESI) m/z: 1171.3 [M+H]⁺.

Step 13: Preparation of tert-butyl 3-[7-(7,8-difluoro-1-naphthyl)-8-fluoro-2-[[(3S,8S)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate and tert-butyl 3-[7-(7,8-difluoro-1-naphthyl)-8-fluoro-2-[[(3S,8S)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

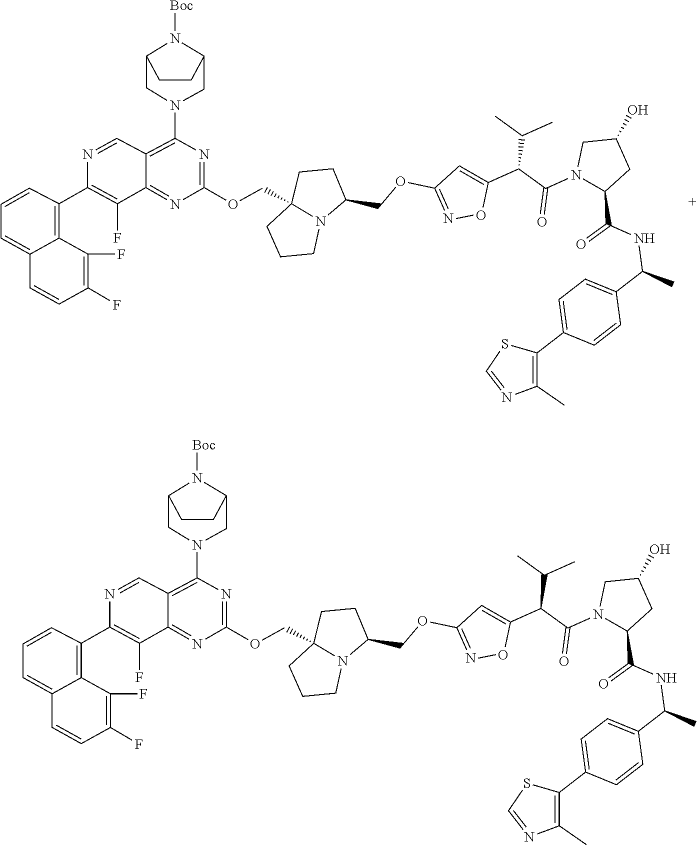 [SMILES: Cc1ncsc1-c1ccc([C@H](C)NC(=O)[C@@H]2C[C@@H](O)CN2C(=O)[C@H](c2cc(OC[C@@H]3CC[C@]4(COc5nc(N6CC7CCC(C6)N7C(=O)OC(C)(C)C)c6cnc(-c7cccc8ccc(F)c(F)c78)c(F)c6n5)CCCN34)no2)C(C)C)cc1 ; Cc1ncsc1-c1ccc([C@H](C)NC(=O)[C@@H]2C[C@@H](O)CN2C(=O)[C@@H](c2cc(OC[C@@H]3CC[C@]4(COc5nc(N6CC7CCC(C6)N7C(=O)OC(C)(C)C)c6cnc(-c7cccc8ccc(F)c(F)c78)c(F)c6n5)CCCN34)no2)C(C)C)cc1]

The epimers were separated by SFC (Instrument: SHIMADZU LC-20AP; Condition: [30% CH₃CN in EtOH (0.1% NH₄OH)]; Gradient time: 15 min; column: DAICEL CHIRALPAK IE (250 mm*30 mm, 10 um; Flow Rate: 80 mL/min). The pure fractions was concentrated under reduced pressure to afford tert-butyl 3-[7-(7,8-difluoro-1-naphthyl)-8-fluoro-2-[[(3S,8S)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (55 mg, 65% purity, 0.031 mmol) as a yellow solid (LC/MS (ESI) m/z: 1171.5 [M+H]; t_{R}=2.471 min), and tert-butyl 3-[7-(7,8-difluoro-1-naphthyl)-8-fluoro-2-[[(3S,8S)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (80 mg, 79.6% purity, 0.054 mmol) as a yellow solid. (LC/MS (ESI) m/z: 1171.5 [M+H]⁺; t_{R}=3.516 min).

Step 14: Preparation of (2S,4R)-1-[(2R)-2-[3-[[(3S,8S)-8-[[4-(3,8-diazabicyclo[3.2.1]octan-3-yl)-7-(7,8-difluoro-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

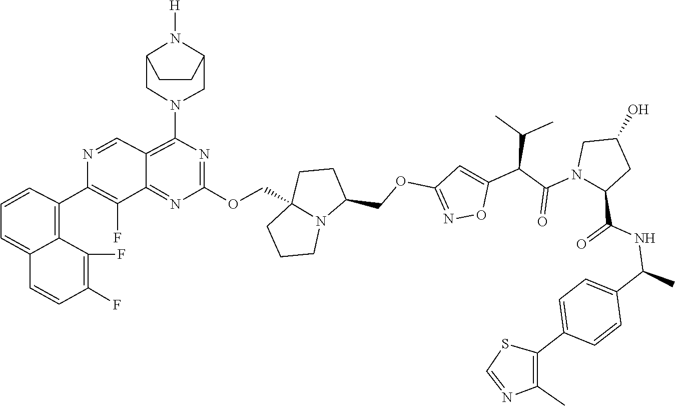 [SMILES: Cc1ncsc1-c1ccc([C@H](C)NC(=O)[C@@H]2C[C@@H](O)CN2C(=O)[C@@H](c2cc(OC[C@@H]3CC[C@]4(COc5nc(N6CC7CCC(C6)N7)c6cnc(-c7cccc8ccc(F)c(F)c78)c(F)c6n5)CCCN34)no2)C(C)C)cc1]

To a solution of tert-butyl 3-[7-(7,8-difluoro-1-naphthyl)-8-fluoro-2-[[(3S,8S)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (80 mg, 0.068 mmol, 1.0 eq) in CH₂Cl₂ (1 mL) was added HCl/dioxane (4 M, 1.0 mL, 58.6 eq), and the reaction mixture was stirred at 20° C. for 0.5 hours. The mixture was diluted with petroleum ether (10 mL) and filtered. The filter cake was dissolved with dichloromethane (20 mL, 0.05% TEA in dichloromethane) and concentrated in vacuum. The resulting residue was purified by prep-HPLC (Condition: [20-50% CH₃CN in water (formic acid)]; Gradient time: 26 min; Column: Phenomenex C18 75*30 mm*3 um; Flow Rate: 25 mL/min). The pure fractions were combined and lyophilized to afford (2S,4R)-1-[(2R)-2-(3-{[(3S,7aS)-7a-{[(4-{3,8-diazabicyclo[3.2.1]octan-3-yl}-7-(7,8-difluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (55.1 mg, 0.048 mmol, 71% yield, 98% purity, formic acid salt) as a white solid. LC/MS (ESI) m/z: 1071.9 [M+H]⁺. 1H NMR (400 MHz, CD₃OD δ 9.17 (s, 1H), 8.90 (s, 1H), 8.52 (s, 1H), 8.19-8.11 (m, 1H), 7.96-7.88 (m, 1H), 7.75-7.66 (m, 2H), 7.61-7.52 (m, 1H), 7.49-7.35 (m, 4H), 6.07-6.00 (m, 1H), 5.08-4.98 (m, 1H), 4.76 (d, J=12.8 Hz, 2H), 4.71-4.56 (m, 4H), 4.55-4.26 (m, 4H), 3.89-3.82 (m, 4H), 3.78-3.67 (m, 1H), 3.61 (d, J=11.2 Hz, 1H), 3.51-3.44 (m, 1H), 3.41-3.34 (m, 1H), 2.53-2.45 (m, 3H), 2.44-2.31 (m, 3H), 2.28-2.09 (m, 5H), 2.09-1.87 (m, 7H), 1.63-1.50 (m, 3H), 1.07 (d, J=6.4 Hz, 3H), 0.94-0.86 (m, 3H).

Exemplary Synthesis of (2S,4R)-1-[(2S)-2-(3-{[(3S,7aS)-7a-{[(4-{3,8-diazabicyclo[3.2.1]octan-3-yl}-7-(7,8-difluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 62)

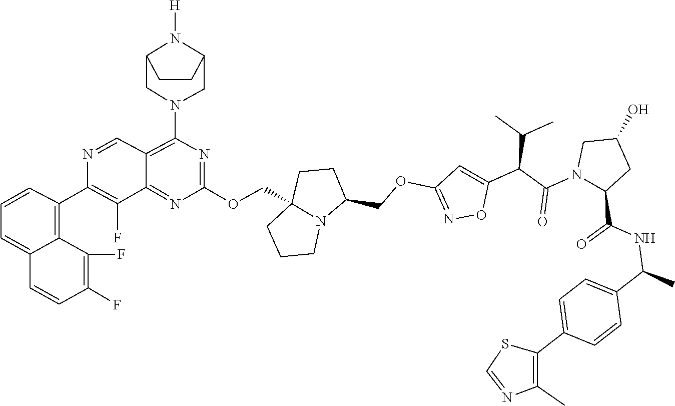 [SMILES: Cc1ncsc1-c1ccc([C@H](C)NC(=O)[C@@H]2C[C@@H](O)CN2C(=O)[C@@H](c2cc(OC[C@@H]3CC[C@]4(COc5nc(N6CC7CCC(C6)N7)c6cnc(-c7cccc8ccc(F)c(F)c78)c(F)c6n5)CCCN34)no2)C(C)C)cc1]

The title compound was prepared in an analogous manner to Compound 61 and purified by prep-HPLC (Condition: [3-33% CH₃CN in water (formic acid)]; Gradient time: 25 min; Column: Phenomenex C18 75*30 mm*3 um; Flow Rate: 25 mL/min). The pure fractions were combined and lyophilized to afford (2S,4R)-1-[(2S)-2-(3-{[(3S,7aS)-7a-{[(4-{3,8-diazabicyclo[3.2.1]octan-3-yl}-7-(7,8-difluoronaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (33.5 mg, 0.029 mmol, 63% yield, formic acid salt) as a white solid. LC/MS (ESI) m/z: 1071.7 [M+H]⁺. ¹H NMR (400 MHz, CD₃OD) δ 9.16 (s, 1H), 8.91-8.81 (m, 1H), 8.47 (s, 1H), 8.17-8.09 (m, 1H), 7.94-7.86 (m, 1H), 7.73-7.65 (m, 2H), 7.58-7.48 (m, 1H), 7.47-7.37 (m, 2H), 7.36-7.30 (m, 2H), 6.07-5.99 (m, 1H), 4.76 (d, J=13.2 Hz, 3H), 4.70 (dd, J=6.0, 12.0 Hz, 2H), 4.63 (d, J=3.6 Hz, 1H), 4.58-4.53 (m, 2H), 4.53-4.47 (m, 1H), 4.43-4.33 (m, 2H), 3.93 (s, 2H), 3.85 (d, J=13.2 Hz, 2H), 3.76 (d, J=9.2 Hz, 1H), 3.73-3.67 (m, 1H), 3.63-3.57 (m, 1H), 3.47 (dd, J=2.4, 6.8 Hz, 1H), 3.37 (d, J=9.6 Hz, 1H), 2.49-2.44 (m, 3H), 2.41-2.30 (m, 3H), 2.28-2.17 (m, 3H), 2.16-2.03 (m, 4H), 2.00 (d, J=13.6 Hz, 2H), 1.92 (d, J=10.0 Hz, 2H), 1.58-1.44 (m, 3H), 1.05 (d, J=6.8 Hz, 3H), 0.95-0.81 (m, 3H).

Exemplary Synthesis of [(3R,7aS)-7a-{[(4-{3,8-diazabicyclo[3.2.1]octan-3-yl}-7-(7,8-difluoronaphthalen-1-yl)-8-fluoropyrido[4,3-d]pyrimidin-2-yl)oxy]methyl}-hexahydro-1H-pyrrolizin-3-yl]methyl 4-{5-[(2R*)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 63)

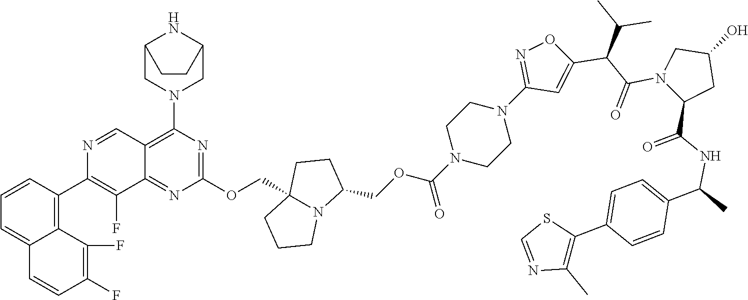 [SMILES: Cc1ncsc1-c1ccc([C@H](C)NC(=O)[C@@H]2C[C@@H](O)CN2C(=O)[C@@H](c2cc(N3CCN(C(=O)OC[C@H]4CC[C@]5(COc6nc(N7CC8CCC(C7)N8)c7cnc(-c8cccc9ccc(F)c(F)c89)c(F)c7n6)CCCN45)CC3)no2)C(C)C)cc1]

The title compound was prepared in an analogous manner to Compound 53 starting from tert-butyl 3-[7-(7,8-difluoro-1-naphthyl)-8-fluoro-2-methylsulfonyl-pyrido[4,3-d]pyrimidin-4-yl]-3,8-diazabicyclo[3.2.1]octane-8-carboxylate and [(3R,8S)-3-[[tert-butyl(diphenyl)silyl]oxymethyl]-1,2,3,5,6,7-hexahydropyrrolizin-8-yl]methanol, and purified by prep-HPLC (column: YMC Triart C18 150*25 mm*5 um; mobile phase: [2-32% CH₃CN in water (formic acid)]) to afford [(3R,7aS)-7a-{[(4-{3,8-diazabicyclo[3.2.1]octan-3-yl}-7-(7,8-difluoronaphthalen-1-yl)-8-fluoropyrido[4,3-d]pyrimidin-2-yl)oxy]methyl}-hexahydro-1H-pyrrolizin-3-yl]methyl 4-{5-[(2R*)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (25.2 mg, 0.02 mmol, 33% yield, 97.8% purity, formic acid salt) as a white solid. LC/MS (ESI) m/z: 1183.2 [M+H]⁺. ¹H NMR (400 MHz, CD₃OD) δ 9.15 (s, 1H), 8.89 (s, 1H), 8.48 (s, 1H), 8.14 (br dd, J=1.2, 2.8 Hz, 1H), 7.99-7.87 (m, 1H), 7.70 (d, J=4.8 Hz, 2H), 7.57 (br d, J=2.0 Hz, 1H), 7.48-7.35 (m, 4H), 6.15-6.04 (m, 1H), 5.06 (br d, J=7.2 Hz, 1H), 4.82-4.74 (m, 5H), 4.60 (br d, J=4.8 Hz, 1H), 4.56-4.42 (m, 3H), 4.33-4.23 (m, 1H), 4.20-4.09 (m, 1H), 4.01-3.78 (m, 4H), 3.65 (br dd, J=10.4, 13.1 Hz, 2H), 3.58-3.41 (m, 4H), 3.21-3.04 (m, 5H), 2.56-2.44 (m, 3H), 2.44-2.29 (m, 2H), 2.23-2.12 (m, 2H), 2.09-1.82 (m, 11H), 1.62-1.48 (m, 3H), 1.07 (br d, J=6.8 Hz, 3H), 0.95-0.81 (m, 3H).

Exemplary Synthesis of (2S,4R)-1-[(2R)-2-(3-{[(3S,7aS)-7a-({[7-(3-chloro-2-cyclopropyl-5-hydroxyphenyl)-4-{3,8-diazabicyclo[3.2.1]octan-3-yl}-8-fluoropyrido[4,3-d]pyrimidin-2-yl]oxy}methyl)-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 64)

Step 1: Preparation of tert-butyl 3-[7-[3-chloro-2-cyclopropyl-5-(methoxymethoxy)phenyl]-8-fluoro-2-methylsulfanyl-pyrido[4,3-d]pyrimidin-4-yl]-3,8-diazabicyclo[3.2.1]octane-8-carboxylate

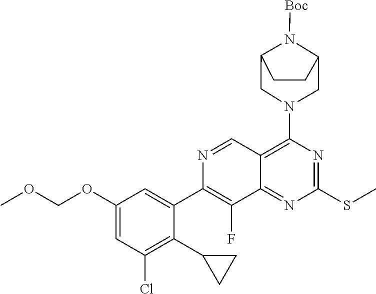

To a solution of 2-[3-chloro-2-cyclopropyl-5-(methoxymethoxy)phenyl]-4,4,5,5-tetramethyl-1,3,2-dioxaborolane (400 mg, 1.18 mmol, 1.0 eq) and tert-butyl 3-(7-chloro-8-fluoro-2-methylsulfanyl-pyrido[4,3-d]pyrimidin-4-yl)-3,8-diazabicyclo[3.2.1]octane-8-carboxylate (520 mg, 1.18 mmol, 1.0 eq) in dioxane (7.5 mL) and H₂O (1.5 mL) were added K₃PO₄ (752 mg, 3.54 mmol, 3.0 eq) and [2-(2-aminophenyl)phenyl]palladium (1+); bis(1-adamantyl)-butyl-phosphane; methanesulfonate (86 mg, 0.118 mmol, 0.1 eq) in one portion at 25° C., and the reaction mixture was stirred at 85° C. for 12 hours under N₂. The residue was partitioned between EtOAc and water (30 mL), and the aqueous phase was extracted with EtOAc (3×50 mL). The combined organic extract was washed with brine (40 mL), dried with anhydrous Na₂SO₄, filtered, and concentrated. The resulting residue was purified by flash chromatography on SiO₂ (gradient: 0˜15% EtOAc in petroleum ether) to afford tert-butyl 3-[7-[3-chloro-2-cyclopropyl-5-(methoxymethoxy)phenyl]-8-fluoro-2-methylsulfanyl-pyrido[4,3-d]pyrimidin-4-yl]-3,8-diazabicyclo[3.2.1]octane-8-carboxylate (342 mg, 0.533 mmol, 45% yield) as a yellow solid. LC/MS (ESI) m/z: 616.1 [M+H]⁺.

Step 2: Preparation of tert-butyl 3-[7-[3-chloro-2-cyclopropyl-5-(methoxymethoxy)phenyl]-8-fluoro-2-methylsulfonyl-pyrido[4,3-d]pyrimidin-4-yl]-3,8-diazabicyclo[3.2.1]octane-8-carboxylate

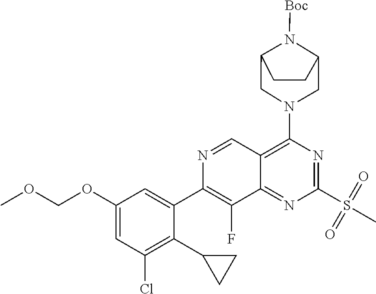

To a solution of tert-butyl 3-[7-[3-chloro-2-cyclopropyl-5-(methoxymethoxy)phenyl]-8-fluoro-2-methylsulfanyl-pyrido[4,3-d]pyrimidin-4-yl]-3,8-diazabicyclo[3.2.1]octane-8-carboxylate (1.6 g, 2.60 mmol, 1.0 eq) in DMF (14 mL) was added 4 Å MS (1.6 g, 1.0 eq), and the resulting mixture was stirred at 40° C. for 12 hours. Oxone (4.8 g, 7.79 mmol, 3.0 eq) was then added, and the reaction mixture was stirred at 40° C. for 24 hours. The mixture was filtered, then aqueous Na₂SO₃ (2×10 mL) was added. The resulting mixture was extracted with EtOAc (3×40 mL), and the combined organic extract was washed with brine (20 mL), dried over anhydrous Na₂SO₄, filtered, and concentrated in vacuum to afford tert-butyl 3-[7-[3-chloro-2-cyclopropyl-5-(methoxymethoxy)phenyl]-8-fluoro-2-methylsulfonyl-pyrido[4,3-d]pyrimidin-4-yl]-3,8-diazabicyclo[3.2.1]octane-8-carboxylate (2.2 g, crude) as a yellow oil. LC/MS (ESI) m/z: 648.0 [M+H].

Step 3: Preparation of (2S,4R)-1-[(2R)-2-(3-{[(3S,7aS)-7a-({[7-(3-chloro-2-cyclopropyl-5-hydroxyphenyl)-4-{3,8-diazabicyclo[3.2.1]octan-3-yl}-8-fluoropyrido[4,3-d]pyrimidin-2-yl]oxy}methyl)-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide

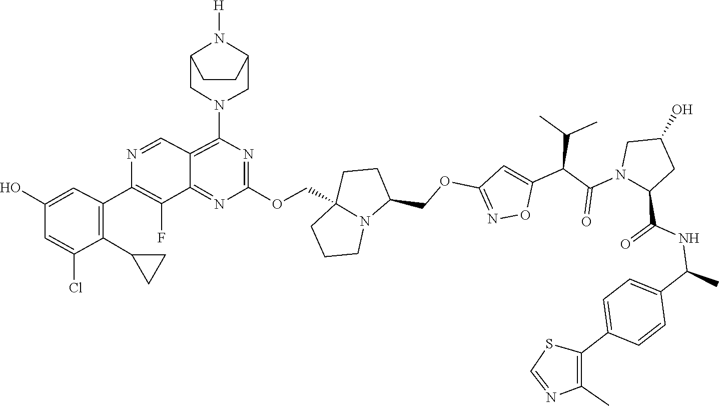 [SMILES: Cc1ncsc1-c1ccc([C@H](C)NC(=O)[C@@H]2C[C@@H](O)CN2C(=O)[C@@H](c2cc(OC[C@@H]3CC[C@]4(COc5nc(N6CC7CCC(C6)N7)c6cnc(-c7cc(O)cc(Cl)c7C7CC7)c(F)c6n5)CCCN34)no2)C(C)C)cc1]

The title compound was prepared in an analogous manner to Compound 61 starting from tert-butyl 3-[7-[3-chloro-2-cyclopropyl-5-(methoxymethoxy)phenyl]-8-fluoro-2-methylsulfonyl-pyrido[4,3-d]pyrimidin-4-yl]-3,8-diazabicyclo[3.2.1]octane-8-carboxylate and (2S,4R)-4-[tert-butyl(diphenyl)silyl]oxy-1-[2-[3-[[(3S,8S)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide, and purified by preparative HPLC [Column: DAICEL CHIRALPAK IC (250 mm*30 mm, 10 um); Mobile phase: [40% CH₃CN in EtOH (0.1% NH₄OH)]; Gradient time: 12 min]. The pure fractions were combined and lyophilized to afford (2S,4R)-1-[(2R)-2-(3-{[(3S,7aS)-7a-({[7-(3-chloro-2-cyclopropyl-5-hydroxyphenyl)-4-{3,8-diazabicyclo[3.2.1]octan-3-yl}-8-fluoropyrido[4,3-d]pyrimidin-2-yl]oxy}methyl)-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide as a white solid. LC/MS (ESI) m/z: 1075.8 [M+H]⁺. ¹H NMR (400 MHz, CD₃OD) δ 9.05 (s, 1H), 8.88 (s, 1H), 7.43 (d, J=5.2 Hz, 3H), 7.27 (d, J=7.2 Hz, 1H), 6.96 (d, J=2.4 Hz, 1H), 6.79 (d, J=2.4 Hz, 1H), 6.02 (s, 1H), 5.06-5.00 (m, 1H), 4.65-4.59 (m, 2H), 4.51 (t, J=8.4 Hz, 1H), 4.47-4.34 (m, 4H), 4.29-4.25 (m, 1H), 3.91-3.82 (m, 1H), 3.76-3.59 (m, 7H), 3.47 (s, 1H), 2.96-2.80 (m, 2H), 2.48 (s, 3H), 2.40-2.34 (m, 1H), 2.23-2.17 (m, 2H), 2.08-2.01 (m, 2H), 1.95-1.83 (m, 8H), 1.52 (d, J=7.2 Hz, 2H), 1.33-1.27 (m, 3H), 1.05 (d, J=6.0 Hz, 3H), 0.89 (d, J=6.8 Hz, 3H), 0.68-0.54 (m, 2H), 0.12-0.01 (m, 2H).

Exemplary Synthesis of (2S,4R)-1-[(2S)-2-(3-{[(3S,7aS)-7a-({[7-(3-chloro-2-cyclopropyl-5-hydroxyphenyl)-4-{3,8-diazabicyclo[3.2.1]octan-3-yl}-8-fluoropyrido[4,3-d]pyrimidin-2-yl]oxy}methyl)-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 65)

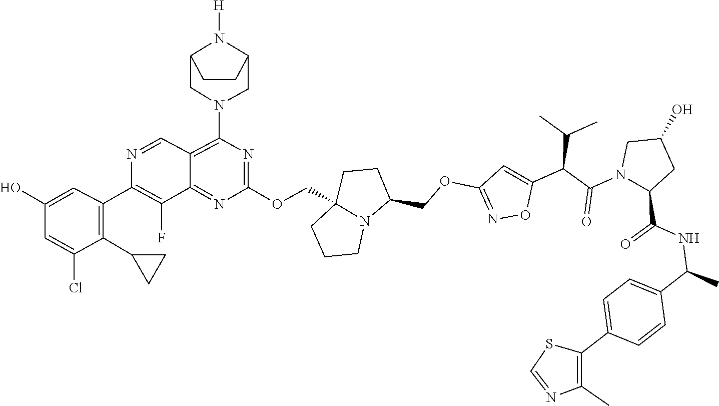 [SMILES: Cc1ncsc1-c1ccc([C@H](C)NC(=O)[C@@H]2C[C@@H](O)CN2C(=O)[C@@H](c2cc(OC[C@@H]3CC[C@]4(COc5nc(N6CC7CCC(C6)N7)c6cnc(-c7cc(O)cc(Cl)c7C7CC7)c(F)c6n5)CCCN34)no2)C(C)C)cc1]

The title compound was prepared in an analogous manner to Compound 64 and purified by preparative HPLC [Column: Phenomenex C18 75*30 mm*3 um; Mobile phase: [20-60% CH₃CN in water (formic acid)]; Gradient time: 25 min]. The pure fractions were combined and lyophilized to afford (2S,4R)-1-[(2S)-2-(3-{[(3S,7aS)-7a-({[7-(3-chloro-2-cyclopropyl-5-hydroxyphenyl)-4-{3,8-diazabicyclo[3.2.1]octan-3-yl}-8-fluoropyrido[4,3-d]pyrimidin-2-yl]oxy}methyl)-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide as a white solid. LC/MS (ESI) m/z: 1075.8 [M+H]⁺. ¹H NMR (400 MHz, CD₃OD) δ 9.04 (s, 1H), 8.81 (S, 1H), 7.36-7.27 (m, 4H), 6.91 (d, J=2.4 Hz, 1H), 6.74 (d, J=2.4 Hz, 1H), 5.96 (s, 1H), 4.90-4.85 (m, 2H), 4.61-4.58 (m, 2H), 4.54 (s, 5H), 4.46-4.40 (m, 2H), 4.38-4.35 (m, 1H), 3.71-3.69 (m, 6H), 3.63-3.59 (m, 1H), 2.41 (s, 3H), 2.30-2.26 (m, 3H), 2.14-1.99 (m, 5H), 1.88-1.79 (m, 7H), 1.41 (d, J=7.2 Hz, 3H), 1.23 (s, 1H), 0.99 (d, J=6.4 Hz, 3H), 0.84 (d, J=4.0 Hz, 3H), 0.63-0.50 (m, 2H), 0.03-0.06 (m, 2H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-2-{[(1S)-1-(4-cyanophenyl)ethyl]carbamoyl}-4-hydroxypyrrolidin-1-yl]-3-methyl-1-oxobutan-2-yl]-1,2-oxazol-3-yl}piperazine-1-carboxylate (Compound 66)

Step 1: Preparation of tert-butyl N-[(1S)-1-(4-bromophenyl)ethyl]carbamate

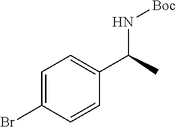 [SMILES: C[C@H](NC(=O)OC(C)(C)C)c1ccc(Br)cc1]

To a solution of (1S)-1-(4-bromophenyl) ethanamine (49.98 mmol, 7.2 mL, 1.0 eq) and TEA (149.94 mmol, 20.8 mL, 3.0 eq) in CH₂Cl₂ (100 mL) at 0° C. was added Boc₂O (59.98 mmol, 13.8 mL, 1.2 eq) dropwise under N₂, and the reaction mixture was stirred at 0° C.-20° C. for 1 hour. The mixture was diluted with water (100 mL) and extracted with CH₂Cl₂ (2×30 mL). The combined organic extracts were washed brine (60 mL), dried over anhydrous Na₂SO₄ and filtered. The organic phase was separated, and the oily solid was concentrated over vacuum. The residue was triturated with petroleum ether/ethyl acetate (110 mL, v/v=10/1) to afford tert-butyl N-[(1S)-1-(4-bromophenyl)ethyl]carbamate (10.38 g, 26.36 mmol, 53% yield) as a white solid. ¹H NMR (400 MHz, CDCl₃) δ 7.48-7.43 (m, 2H), 7.18 (br d, J=8.4 Hz, 2H), 4.88-4.54 (m, 2H), 1.42 (brs, 12H).

Step 2: Preparation of tert-butyl N-[(1S)-1-(4-cyanophenyl)ethyl]carbamate

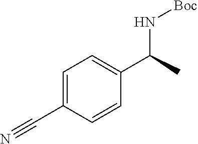 [SMILES: C[C@H](NC(=O)OC(C)(C)C)c1ccc(C#N)cc1]

To a solution of tert-butyl N-[(1S)-1-(4-bromophenyl)ethyl]carbamate (10.38 g, 26.36 mmol, 76.22% purity, 1.0 eq) in DMF (110 mL) were added Zn(CN)₂ (5.26 g, 44.79 mmol, 1.7 eq) and Pd(PPh₃)₄ (2.8 g, 2.42 mmol, 0.01 eq) under N₂, and the reaction mixture was stirred at 105° C. for 16 hours under N₂. The mixture was diluted with methyl tert-butyl ether (110 mL), washed with water (3×110 mL) and brine (200 mL), dried over anhydrous Na₂SO₄, filtered, and concentrated under reduced pressure. The crude product was purified by flash chromatography on SiO₂ (gradient: 0˜12% EtOAc in petroleum ether) to afford tert-butyl N-[(1S)-1-(4-cyanophenyl)ethyl]carbamate (5.53 g, 21.15 mmol, 80% yield) as a yellow oil. LC/MS (ESI) m/z: 247.1 [M+H]⁺.

Step 3: Preparation of 4-[(1S)-1-aminoethyl]benzonitrile

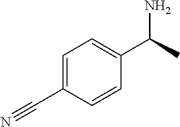

To a solution of tert-butyl N-[(1S)-1-(4-cyanophenyl)ethyl]carbamate (5.53 g, 21.15 mmol, 1.0 eq) in CH₂Cl₂ (50 mL) was added HCl/dioxane (4 M, 52.9 mL, 10 eq), and the reaction mixture was stirred at 20° C. for 1 hour. The organic phase was separated, the oily solid was concentrated to give 4-[(1S)-1-aminoethyl]benzonitrile (3.86 g, crude HCl salt) as a white solid. LC/MS (ESI) m/z: 147.0 [M+H]⁺.

Step 4: Preparation of tert-butyl(2S,4R)-2-[[(1S)-1-(4-cyanophenyl)ethyl]carbamoyl]-4-hydroxy-pyrrolidine-1-carboxylate

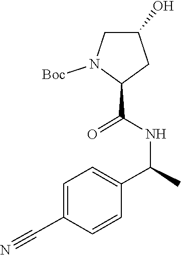 [SMILES: C[C@H](NC(=O)[C@@H]1C[C@@H](O)CN1C(=O)OC(C)(C)C)c1ccc(C#N)cc1]

To a solution of 4-[(1S)-1-aminoethyl]benzonitrile (3.89 g, crude HCl salt, 20.67 mmol, 1.0 eq) and (2S,4R)-1-tert-butoxycarbonyl-4-hydroxy-pyrrolidine-2-carboxylic acid (5.3 g, 22.91 mmol, 1.1 eq) in CH₂Cl₂ (80 mL) were added DIEA (104.15 mmol, 18.1 mL, 5.0 eq) and HATU (8.7 g, 22.91 mmol, 1.1 eq), and the reaction mixture was stirred at 25° C. for 16 hours. The mixture was washed with water (80 mL), saturated aqueous NaHCO₃ (40 mL) and brine (70 mL), dried over Na₂SO₄, filtered, and concentrated. The residue was purified by flash chromatography on SiO₂ (gradient: 0˜82% EtOAc in petroleum ether) to afford tert-butyl (2S,4R)-2-[[(1S)-1-(4-cyanophenyl)ethyl]carbamoyl]-4-hydroxy-pyrrolidine-1-carboxylate (7.49 g, 17.4 mmol, 84% yield) as a yellow foam. LC/MS (ESI) m/z: 360.0 [M+H]⁺. ¹H NMR (400 MHz, CDCl₃) δ 7.64-7.58 (m, 2H), 7.43 (d, J=8.4 Hz, 2H), 4.58-4.34 (m, 2H), 3.78-3.64 (m, 2H), 3.54-3.46 (m, 2H), 3.23-3.14 (m, 1H), 2.83 (s, 1H), 1.49-1.43 (m, 12H), 1.21-1.21 (m, 1H).

Step 5: Preparation of (2S,4R)—N-[(1S)-1-(4-cyanophenyl)ethyl]-4-hydroxy-pyrrolidine-2-carboxamide

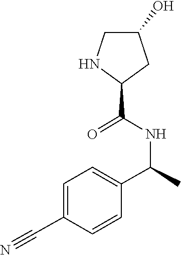 [SMILES: C[C@H](NC(=O)[C@@H]1C[C@@H](O)CN1)c1ccc(C#N)cc1]

To a solution of tert-butyl(2S,4R)-2-[[(1S)-1-(4-cyanophenyl)ethyl]carbamoyl]-4-hydroxy-pyrrolidine-1-carboxylate (7.49 g, 83.5% purity, 17.40 mmol, 1.0 eq) in CH₂Cl₂ (70 mL) was added HCl/dioxane (4 M, 43.5 mL, 10 eq), and the reaction mixture was stirred at 20° C. for 1 hour. The organic phase was separated and filtered. The oily solid was triturated with petroleum ether/ethyl acetate (110 mL, V:V=10:1) and concentrated over vacuum to afford (2S,4R)—N-[(1S)-1-(4-cyanophenyl)ethyl]-4-hydroxy-pyrrolidine-2-carboxamide (9.0 g, crude HCl salt) as a pink solid. LC/MS (ESI) m/z: 260.2 [M+H]⁺. ¹H NMR (400 MHz, CDCl₃) δ 10.20-9.95 (m, 1H), 9.40-9.26 (m, 1H), 8.71-8.47 (m, 1H), 7.81 (d, J=8.4 Hz, 2H), 7.50 (d, J=8.8 Hz, 2H), 5.00 (quint, J=7.2 Hz, 1H), 4.42-4.32 (m, 2H), 3.65-3.57 (m, 1H), 3.37-3.21 (m, 1H), 3.17-3.04 (m, 2H), 2.41-2.30 (m, 1H), 1.82-1.69 (m, 1H), 1.40 (d, J=6.8 Hz, 3H).

Step 6: Preparation of tert-butyl 4-[[5-[1-[(2S,4R)-2-[[(1S)-1-(4-cyanophenyl)ethyl]carbamoyl]-4-hydroxy-pyrrolidine-1-carbonyl]-2-methyl-propyl]isoxazol-3-yl]piperazine-1-carboxylate

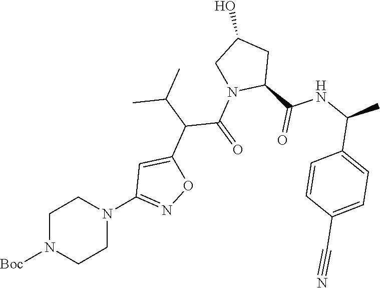 [SMILES: CC(C)C(C(=O)N1C[C@H](O)C[C@H]1C(=O)N[C@@H](C)c1ccc(C#N)cc1)c1cc(N2CCN(C(=O)OC(C)(C)C)CC2)no1]

To a solution of (2S,4R)—N-[(1S)-1-(4-cyanophenyl)ethyl]-4-hydroxy-pyrrolidine-2-carboxamide (1.6 g, 57.2% purity, 3.58 mmol, 1.1 eq) and 2-[3-(4-tert-butoxycarbonyl piperazin-1-yl)isoxazol-5-yl]-3-methyl-butanoic acid (1.2 g, 3.25 mmol, 1.0 eq) in CH₂Cl₂ (25 mL) were added DIEA (2.8 mL, 16.27 mmol, 5.0 eq) and HATU (1.4 g, 3.58 mmol, 1.1 eq), and the reaction mixture was stirred at 25° C. for 16 hours. The mixture was washed with water (40 mL), saturated aqueous NaHCO₃ (20 mL) and brine (40 mL), dried over Na₂SO₄, filtered, and concentrated. The residue was purified by flash chromatography on SiO₂ (gradient: 0˜86% EtOAc in petroleum ether) to afford tert-butyl 4-[[5-[1-[(2S,4R)-2-[[(1S)-1-(4-cyanophenyl)ethyl]carbamoyl]-4-hydroxy-pyrrolidine-1-carbonyl]-2-methyl-propyl]isoxazol-3-yl]piperazine-1-carboxylate (1.66 g, 2.33 mmol, 72% yield) as a yellow foam. LC/MS (ESI) m/z: 495.2 [M+H]⁺. ¹H NMR (400 MHz, DMSO-d₆) δ 8.51-8.27 (m, 1H), 7.85-7.70 (m, 2H), 7.54-7.37 (m, 2H), 6.20-6.12 (m, 1H), 5.16-5.08 (m, 1H), 4.95-4.83 (m, 1H), 4.43-4.31 (m, 1H), 4.29-4.19 (m, 1H), 3.74-3.64 (m, 1H), 3.63-3.51 (m, 2H), 3.15-3.10 (m, 4H), 2.28-2.08 (m, 1H), 2.07-1.99 (m, 1H), 1.80-1.67 (m, 1H), 1.41 (d, J=1.2 Hz, 9H), 1.37-1.30 (m, 3H), 1.26-1.24 (m, 4H), 0.98-0.89 (m, 3H), 0.83-0.76 (m, 3H).

Step 7: Preparation of tert-butyl 4-[[5-[(1S)-1-[(2S,4R)-2-[[(1S)-1-(4-cyanophenyl)ethyl]carbamoyl]-4-hydroxy-pyrrolidine-1-carbonyl]-2-methyl-propyl]isoxazol-3-yl]piperazine-1-carboxylate & tert-butyl 4-[[5-[(1R)-1-[(2S,4R)-2-[[(1S)-1-(4-cyanophenyl)ethyl]carbamoyl]-4-hydroxy-pyrrolidine-1-carbonyl]-2-methyl-propyl]isoxazol-3-yl]piperazine-1-carboxylate

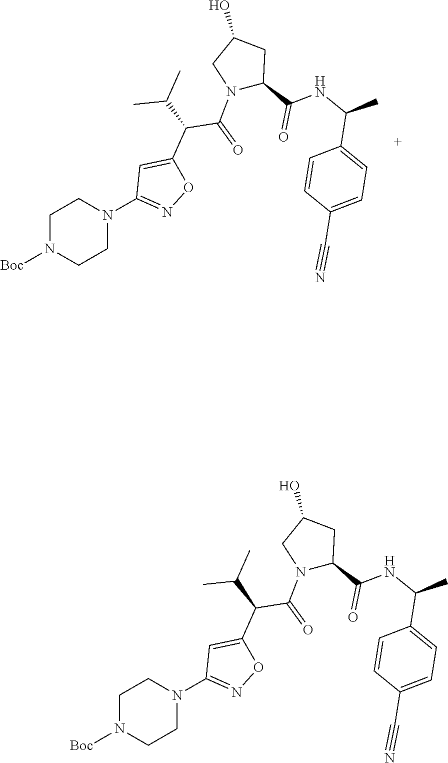 [SMILES: CC(C)[C@H](C(=O)N1C[C@H](O)C[C@H]1C(=O)N[C@@H](C)c1ccc(C#N)cc1)c1cc(N2CCN(C(=O)OC(C)(C)C)CC2)no1 ; CC(C)[C@@H](C(=O)N1C[C@H](O)C[C@H]1C(=O)N[C@@H](C)c1ccc(C#N)cc1)c1cc(N2CCN(C(=O)OC(C)(C)C)CC2)no1]

Tert-butyl 4-[[5-[1-[(2S,4R)-2-[[(1S)-1-(4-cyanophenyl)ethyl]carbamoyl]-4-hydroxy-pyrrolidine-1-carbonyl]-2-methyl-propyl]isoxazol-3-yl]piperazine-1-carboxylate (980 g, 83.6% purity, 1.26 mmol) was purified by SFC (Column: Daicel ChiralPak IG (250*30 mm, 10 um); Mobile phase: [0.1% NH₄OH in EtOH]; B %: 35%-35%; Hold time: 25 min). Fraction 1 was concentrated under reduced pressure to afford tert-butyl 4-[[5-[(1S)-1-[(2S,4R)-2-[[(1S)-1-(4-cyanophenyl)ethyl]carbamoyl]-4-hydroxy-pyrrolidine-1-carbonyl]-2-methyl-propyl]isoxazol-3-yl]piperazine-1-carboxylate (533.5 mg, 0.837 mol) as a yellow oil. LC/MS (ESI) m/z: 595.3 [M+H]⁺. Fraction 2 was concentrated under reduced pressure to afford tert-butyl 4-[[5-[(1R)-1-[(2S,4R)-2-[[(1S)-1-(4-cyanophenyl)ethyl]carbamoyl]-4-hydroxy-pyrrolidine-1-carbonyl]-2-methyl-propyl]isoxazol-3-yl]piperazine-1-carboxylate (351.6 mg, 0.54 mmol) as a white solid. LC/MS (ESI) m/z: 595.2 [M+H]⁺.

Step 8: Preparation of (2S,4R)—N-[(1S)-1-(4-cyanophenyl)ethyl]-4-hydroxy-1-[(2R)-3-methyl-2-(3-piperazin-1-ylisoxazol-5-yl) butanoyl]pyrrolidine-2-carboxamide

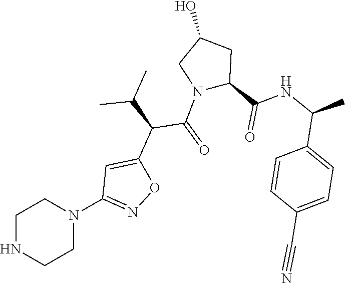 [SMILES: CC(C)[C@@H](C(=O)N1C[C@H](O)C[C@H]1C(=O)N[C@@H](C)c1ccc(C#N)cc1)c1cc(N2CCNCC2)no1]

To a solution of tert-butyl 4-[[5-[(1R)-1-[(2S,4R)-2-[[(1S)-1-(4-cyanophenyl)ethyl]carbamoyl]-4-hydroxy-pyrrolidine-1-carbonyl]-2-methyl-propyl]isoxazol-3-yl]piperazine-1-carboxylate (227.6 mg, 0.383 mmol, 1.0 eq) in CH₂Cl₂ (2 mL) was added HCl/dioxane (4 M, 2 mL), and the reaction mixture was stirred at 25° C. for 1 hour. The mixture was concentrated to afford (2S,4R)—N-[(1S)-1-(4-cyanophenyl)ethyl]-4-hydroxy-1-[(2R)-3-methyl-2-(3-piperazin-1-ylisoxazol-5-yl) butanoyl]pyrrolidine-2-carboxamide (189.3 mg, 0.344 mmol, 90% yield, HCl salt) as a yellow solid. LC/MS (ESI) m/z: 495.3 [M+H]⁺.

Step 9: Preparation of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-4-{5-[(2R)-1-[(2S,4R)-2-{[(1S)-1-(4-1H-pyrrolizin-3-yl]methyl cyanophenyl)ethyl]carbamoyl}-4-hydroxypyrrolidin-1-yl]-3-methyl-1-oxobutan-2-yl]-1,2-oxazol-3-yl}piperazine-1-carboxylate

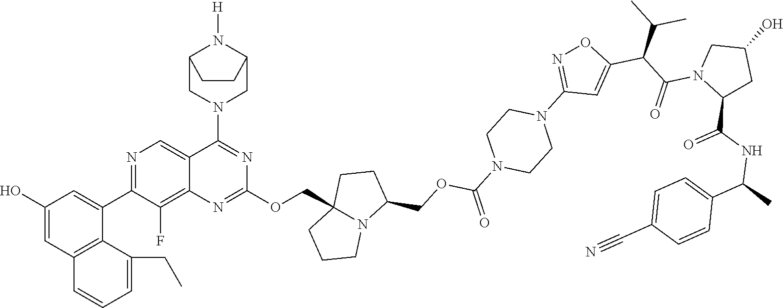 [SMILES: CCc1cccc2cc(O)cc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(C#N)cc8)C(C)C)on7)CC5)CC6)nc4c3F)c12]

The title compound was prepared in an analogous manner to Compound 56 starting from tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)—N-[(1S)-1-(4-cyanophenyl)ethyl]-4-hydroxy-1-[(2R)-3-methyl-2-(3-piperazin-1-ylisoxazol-5-yl) butanoyl]pyrrolidine-2-carboxamide, and purified by preparative HPLC (Column: Phenomenex C18 75*30 mm*3 um; Eluent: 3-43% CH₃CN in water (formic acid); Gradient time: 26 min; Hold time: 4 min; Flow rate: 25 mL/min). The pure fractions were combined and concentrated under reduced pressure, then lyophilized to afford [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-2-{[(1S)-1-(4-cyanophenyl)ethyl]carbamoyl}-4-hydroxypyrrolidin-1-yl]-3-methyl-1-oxobutan-2-yl]-1,2-oxazol-3-yl}piperazine-1-carboxylate (121.4 mg, 0.106 mmol, 60% yield) as a white solid. LC/MS (ESI) m/z: 1119.9 [M+H]⁺. ¹H NMR (400 MHz, CD₃OD) δ 9.16-9.10 (m, 1H), 8.39 (s, 1H), 7.71-7.57 (m, 3H), 7.46-7.34 (m, 3H), 7.30 (dd, J=7.6, 2.8, Hz, 1H), 7.19-7.14 (m, 1H), 7.03 (dd, J=9.6, 2.4 Hz, 1H), 6.10-6.03 (m, 1H), 5.05-4.99 (m, 1H), 4.82-4.70 (m, 3H), 4.56-4.45 (m, 3H), 4.44-4.29 (m, 2H), 4.23-4.14 (m, 1H), 4.08-3.99 (m, 2H), 3.94-3.77 (m, 3H), 3.66-3.58 (m, 2H), 3.57-3.44 (m, 5H), 3.23-3.07 (m, 5H), 2.40-1.90 (m, 18H), 1.53-1.42 (m, 3H), 1.08-1.00 (m, 3H), 0.93-0.85 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2S)-1-[(2S,4R)-2-{[(1S)-1-(4-cyanophenyl)ethyl]carbamoyl}-4-hydroxypyrrolidin-1-yl]-3-methyl-1-oxobutan-2-yl]-1,2-oxazol-3-yl}piperazine-1-carboxylate (Compound 67)

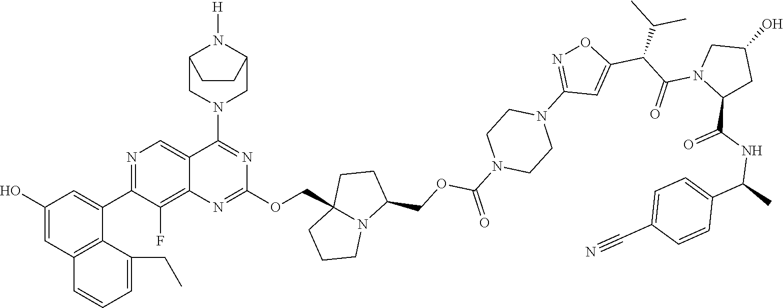 [SMILES: CCc1cccc2cc(O)cc(-c3ncc4c(N5CC6CCC(C5)N6)nc(OC[C@]56CCCN5[C@H](COC(=O)N5CCN(c7cc([C@@H](C(=O)N8C[C@H](O)C[C@H]8C(=O)N[C@@H](C)c8ccc(C#N)cc8)C(C)C)on7)CC5)CC6)nc4c3F)c12]

The title compound was prepared in an analogous manner to Compound 66 starting from tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)—N-[(1S)-1-(4-cyanophenyl)ethyl]-4-hydroxy-1-[(2S)-3-methyl-2-(3-piperazin-1-ylisoxazol-5-yl) butanoyl]pyrrolidine-2-carboxamide, and purified by prep-HPLC (Column: Phenomenex C18 75*30 mm*3 um; Eluent: 3-43% CH₃CN in water (formic acid); Gradient time: 28 min; Hold time: 4 min; Flow rate: 25 mL/min). The pure fractions were combined and concentrated under reduced pressure, then lyophilized to afford [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2S)-1-[(2S,4R)-2-{[(1S)-1-(4-cyanophenyl)ethyl]carbamoyl}-4-hydroxypyrrolidin-1-yl]-3-methyl-1-oxobutan-2-yl]-1,2-oxazol-3-yl}piperazine-1-carboxylate (97.7 mg, 0.086 mmol, 61% yield) as a white solid. LC/MS (ESI) m/z: 1120.0 [M+H]⁺. ¹H NMR (400 MHz, CD₃OD) δ 9.15-9.07 (m, 1H), 8.48-8.39 (m, 2H), 7.73-7.58 (m, 3H), 7.52-7.33 (m, 3H), 7.31-7.25 (m, 1H), 7.16 (d, J=7.2 Hz, 1H), 7.02 (t, J=2.4 Hz, 1H), 6.15-6.06 (m, 1H), 4.76 (d, J=14.4 Hz, 3H), 4.59-4.50 (m, 3H), 4.43-4.31 (m, 2H), 4.24-4.16 (m, 1H), 4.09 (d, J=8.8 Hz, 2H), 3.96-3.82 (m, 2H), 3.78-3.62 (m, 3H), 3.60-3.35 (m, 7H), 3.21-3.10 (m, 2H), 3.20-3.10 (m, 1H), 2.41-1.90 (m, 18H), 1.44 (dd, J=7.2, 4.0 Hz, 3H), 1.05 (d, J=6.4 Hz, 3H), 0.94-0.85 (m, 6H).

Exemplary Synthesis of (2S,4R)-1-[(2R*)-2-[3-(7-{2-[(4-{3,8-diazabicyclo[3.2.1]octan-3-yl}-7-(7,8-difluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 68)

Step 1: Preparation of 1,2-difluoro-8-(methoxymethoxy) naphthalene

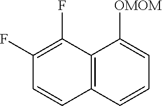

To a solution of 7,8-difluoronaphthalen-1-ol (10 g, 55.51 mmol, 1 eq) in dichloromethane (120 mL) at 0° C. were added N,N-diisopropylethylamine (21.52 g, 166.53 mmol, 29.0 mL, 3 eq) and chloromethyl methyl ether (8.24 g, 102.37 mmol, 7.8 mL, 1.84 eq), and the reaction mixture was stirred at 20° C. for 2 hours. The solution was poured into water (200 mL) slowly and extracted with dichloromethane (2×80 mL). The organic extract was dried over anhydrous sodium sulfate, filtered, and concentrated under vacuum. The resulting residue was purified by silica gel column chromatography (gradient: 0-10% EtOAc in petroleum ether) to afford 1,2-difluoro-8-(methoxymethoxy) naphthalene (11 g, 49.06 mmol, 88% yield) as a white solid. ¹H NMR (400 MHz, CDCl₃) δ 7.55 (ddd, J=2.0, 4.8, 9.2 Hz, 1H), 7.46 (dd, J=1.2, 8.0 Hz, 1H), 7.40-7.29 (m, 2H), 7.17 (d, J=7.6 Hz, 1H), 5.37 (s, 2H), 3.59 (s, 3H).

Step 2: Preparation of 2-[5,6-difluoro-4-(methoxymethoxy)-2-naphthyl]-4,4,5,5-tetramethyl-1,3,2-dioxaborolane

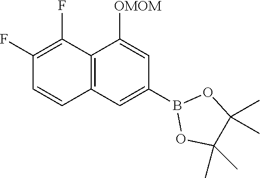

To a solution of 1,2-difluoro-8-(methoxymethoxy) naphthalene (10 g, 44.60 mmol, 1 eq) and 4,4,5,5-tetramethyl-2-(4,4,5,5-tetramethyl-1,3,2-dioxaborolan-2-yl)-1,3,2-dioxaborolane (7.93 g, 31.22 mmol, 0.7 eq) in THF (120 mL) were added (1Z,5Z)-cycloocta-1,5-diene; 2,4-dimethyl-BLAHbicyclo[1.1.0]butane (2.96 g, 4.46 mmol, 0.1 eq) and 4-tert-butyl-2-(4-tert-butyl-2-pyridyl)pyridine (1.44 g, 5.35 mmol, 0.12 eq), and the reaction mixture was stirred at 60° C. for 12 hours. The mixture was cooled to 20° C., then filtered trough a pad of silica gel (100˜200 mesh). The filtrate was concentrated under vacuum to afford 2-[5,6-difluoro-4-(methoxymethoxy)-2-naphthyl]-4,4,5,5-tetramethyl-1,3,2-dioxaborolane (16.9 g, crude) as a black oil.

Step 3: Preparation of 5,6-difluoro-4-(methoxymethoxy) naphthalen-2-ol

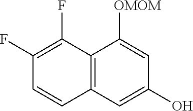

To a solution of 2-[5,6-difluoro-4-(methoxymethoxy)-2-naphthyl]-4,4,5,5-tetramethyl-1,3,2-dioxaborolane (16.9 g, 48.26 mmol, 1 eq) in THF (170 mL) at 0° C. were added acetic acid (202.88 g, 3.38 mol, 193.2 mL, 70 eq) and hydrogen peroxide (46.33 g, 408.62 mmol, 39.26 mL, 30% purity, 8.47 eq), and the reaction mixture was stirred at 20° C. for 1 hour. The reaction was quenched with saturated aqueous sodium thiosulfate (400 mL) and stirred at 20° C. for 1 hour. The resulting mixture was extracted with EtOAc (2×150 mL), and the combined organic extract was dried over anhydrous sodium sulfate, filtered, and concentrated. The resulting residue was purified by silica gel column chromatography (gradient: 0-10% EtOAc in petroleum ether) to afford 5,6-difluoro-4-(methoxymethoxy) naphthalen-2-ol (950 mg, 3.95 mmol, 8% yield) as a brown solid. ¹H NMR (400 MHz, DMSO-d₆) δ 7.37-7.29 (m, 2H), 7.10 (dd, J=2.0, 8.0 Hz, 1H), 7.07-6.97 (m, 1H), 5.82 (br s, 1H), 5.36 (s, 2H), 3.60 (s, 3H).

Step 4: Preparation of [5,6-difluoro-4-(methoxymethoxy)-2-naphthyl]acetate

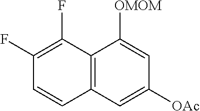

To a solution of 5,6-difluoro-4-(methoxymethoxy) naphthalen-2-ol (950 mg, 3.95 mmol, 1 eq) in dichloromethane (5 mL) at 0° C. were added triethylamine (800 mg, 7.91 mmol, 1.1 mL, 2 eq) and 4-dimethylaminopyridine (48 mg, 0.40 mmol, 0.1 eq) followed by acetyl chloride (621 mg, 7.91 mmol, 0.6 mL, 2 eq) in dichloromethane (4 mL) dropwise, and the reaction mixture was stirred at 20° C. for 0.5 hours. Water (30 mL) was then added, and the resulting mixture was extracted with dichloromethane (2×30 mL2). The combined organic extract was dried over anhydrous sodium sulfate, filtered, and concentrated under vacuum. The resulting residue was purified by silica gel column chromatography (gradient: 0-10% EtOAc in petroleum ether) to afford [5,6-difluoro-4-(methoxymethoxy)-2-naphthyl]acetate (860 mg, 3.05 mmol, 77% yield) as a light yellow gum. ¹H NMR (400 MHz, CDCl₃) δ 7.48 (ddd, J=2.0, 4.8, 9.2 Hz, 1H), 7.34 (dt, J=7.2, 9.6 Hz, 1H), 7.22 (t, J=1.6 Hz, 1H), 6.95 (d, J=2.0 Hz, 1H), 5.36 (s, 2H), 3.58 (s, 3H), 2.35 (s, 3H).

Step 5: Preparation of (5,6-difluoro-4-hydroxy-2-naphthyl)acetate

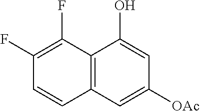 [SMILES: CC(=O)Oc1cc(O)c2c(F)c(F)ccc2c1]

To a solution of [5,6-difluoro-4-(methoxymethoxy)-2-naphthyl]acetate (860 mg, 3.05 mmol, 1 eq) in EtOAc (2 mL) at −40° C. was added HCl in EtOAc (4 M, 9 mL, 11.81 eq), and the reaction mixture was stirred at −40° C. for 0.5 hour, then at 0° C. for 1 hour. The mixture was diluted with saturated aqueous sodium bicarbonate (30 mL) and extracted with EtOAc (2×20 mL). The combined organic extract was washed with brine (20 mL), dried over anhydrous sodium sulfate, filtered, and concentrated under vacuum to afford (5,6-difluoro-4-hydroxy-2-naphthyl)acetate (730 mg, crude) as a light yellow gum.

Step 6: Preparation of [5,6-difluoro-4-(trifluoromethylsulfonyloxy)-2-naphthyl]acetate

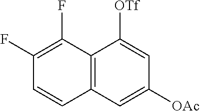 [SMILES: CC(=O)Oc1cc(OS(=O)(=O)C(F)(F)F)c2c(F)c(F)ccc2c1]

To a solution of (5,6-difluoro-4-hydroxy-2-naphthyl)acetate (730 mg, 3.06 mmol, 1 eq) and N,N-diisopropylethylamine (1.19 g, 9.19 mmol, 1.6 mL, 3 eq) in dichloromethane (7 mL) at −65° C. was added trifluoromethanesulfonic anhydride (1.04 g, 3.68 mmol, 0.6 mL, 1.2 eq), and the reaction mixture was stirred at −65° C. for 0.5 hours. Water (20 mL) was then added, and the resulting mixture was extracted with dichloromethane (2×20 mL). The combined organic extract was dried over anhydrous sodium sulfate, filtered, and concentrated under vacuum. The resulting residue was purified by silica gel column chromatography (gradient: 0-10% in petroleum EtOAc ether) to afford [5,6-difluoro-4-(trifluoromethylsulfonyloxy)-2-naphthyl]acetate (340 mg, 0.92 mmol, 30% yield) as a light yellow solid. ¹H NMR (400 MHz, CDCl₃) δ 7.68 (s, 1H), 7.65 (ddd, J=1.6, 4.4, 9.2 Hz, 1H), 7.57-7.44 (m, 1H), 7.36 (d, J=1.6 Hz, 1H), 2.39 (s, 3H).

Step 7: Preparation of (7,8-difluoro-3-hydroxy-1-naphthyl)trifluoromethanesulfonate

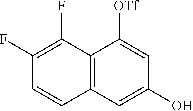 [SMILES: O=S(=O)(Oc1cc(O)cc2ccc(F)c(F)c12)C(F)(F)F]

To a mixture of [5,6-difluoro-4-(trifluoromethylsulfonyloxy)-2-naphthyl]acetate (450 mg, 1.22 mmol, 1 eq) (61 mg, 1.46 mmol, 1.2 eq) in tetrahydrofuran (3 mL) and water (1 mL) 0° C. was added LiOH·H₂O, and the reaction mixture was stirred at 0° C. for 0.5 hour. The mixture was adjusted the pH ˜6 with acetic acid. Water (20 mL) was then added, and the resulting mixture was extracted with EtOAc (2×20 mL). The combined organic extract was dried over anhydrous sodium sulfate, filtered, and concentrated under vacuum to afford (7,8-difluoro-3-hydroxy-1-naphthyl)trifluoromethanesulfonate (360 mg, crude) as a brown solid.

Step 8: Preparation of [7,8-difluoro-3-(methoxymethoxy)-1-naphthyl] trifluoromethanesulfonate

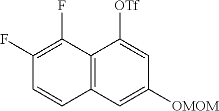

To a mixture of (7,8-difluoro-3-hydroxy-1-naphthyl)trifluoromethanesulfonate (360 mg, 1.10 mmol, 1 eq) in dichloromethane (5 mL) at 0° C. was added N,N-diisopropylethylamine (425 mg, 3.29 mmol, 0.6 mL, 3 eq) followed by chloromethyl methyl ether (520 mg, 6.46 mmol, 0.5 mL, 5.89 eq) dropwise, and the reaction mixture was stirred at 0° C. for 1 hour. Water (30 mL) was then added, and the resulting mixture was extracted with EtOAc (2×20 mL).

The combined organic extract was washed with brine (20 mL), dried over anhydrous sodium sulfate, filtered, and concentrated under vacuum. The resulting residue was purified by silica gel column chromatography (gradient: 0-10% EtOAc in petroleum ether) to afford [7,8-difluoro-3-(methoxymethoxy)-1-naphthyl] trifluoromethanesulfonate (350 mg, 0.94 mmol, 86% yield) as a light yellow oil. ¹H NMR (400 MHz, CDCl₃) δ 7.60-7.52 (m, 1H), 7.45 (s, 1H), 7.43-7.37 (m, 1H), 7.29 (s, 1H), 5.30 (s, 2H), 3.53 (s, 3H).

Step 9: Preparation of 2-[7,8-difluoro-3-(methoxymethoxy)-1-naphthyl]-4,4,5,5-tetramethyl-1,3,2-dioxaborolane

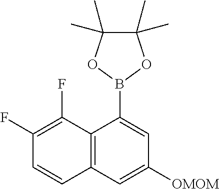

To a solution of [7,8-difluoro-3-(methoxymethoxy)-1-naphthyl] trifluoromethanesulfonate (350 mg, 0.94 mmol, 1 eq) and 4,4,5,5-tetramethyl-2-(4,4,5,5-tetramethyl-1,3,2-dioxaborolan-2-yl)-1,3,2-dioxaborolane (478 mg, 1.88 mmol, 2 eq) in N,N-dimethylformamide (4 mL) were added potassium acetate (461 mg, 4.70 mmol, 5 eq) and [1,1′-bis(diphenylphosphino)ferrocene]dichloropalladium (II). CH₂Cl₂ (77 mg, 0.09 mmol, 0.1 eq), and the reaction mixture was stirred at 80° C. for 12 hours. The mixture was cooled to 25° C., water (30 mL) was then added, and the resulting mixture was extracted with EtOAc (2×20 mL). The combined organic extract was washed with brine (15 mL), dried over anhydrous sodium sulfate, filtered, and concentrated under vacuum. The resulting residue was purified by silica gel column chromatography (gradient: 0-3% EtOAc in petroleum ether) to afford 2-[7,8-difluoro-3-(methoxymethoxy)-1-naphthyl]-4,4,5,5-tetramethyl-1,3,2-dioxaborolane (210 mg, 0.60 mmol, 64% yield) as colorless gum. ¹H NMR (400 MHz, CDCl₃) δ 7.47 (ddd, J=1.2, 4.8, 9.2 Hz, 1H), 7.43 (d, J=2.0 Hz, 1H), 7.40 (d, J=2.0 Hz, 1H), 7.34-7.28 (m, 1H), 5.29 (s, 2H), 3.51 (s, 3H), 1.45 (s, 12H).

Step 10: Preparation of tert-butyl 3-[7-[7,8-difluoro-3-(methoxymethoxy)-1-naphthyl]-2-(2,2-dimethoxyethoxy)-8-fluoro-pyrido[4,3-d]pyrimidin-4-yl]-3,8-diazabicyclo[3.2.1]octane-8-carboxylate

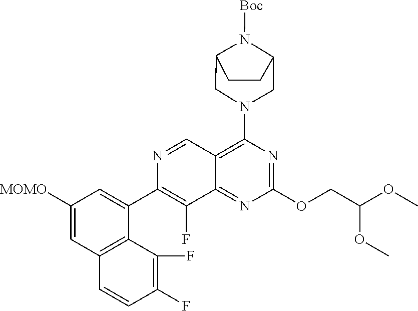

A mixture of 2-[7,8-difluoro-3-(methoxymethoxy)-1-naphthyl]-4,4,5,5-tetramethyl-1,3,2-dioxaborolane (210 mg, 0.60 mmol, 1 eq) and tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (299 mg, 0.60 mmol, 1 eq), potassium phosphate (381.90 mg, 1.80 mmol, 3 eq), and [2-(2-aminophenyl)phenyl]palladium (1+); bis(1-adamantyl)-butyl-phosphane; methanesulfonate (43 mg, 0.06 mmol, 0.1 eq) in dioxane (5 mL) and water (0.5 mL) was degassed and purged with nitrogen (3×). The reaction mixture was then stirred at 90° C. for 12 hours. The mixture was cooled to 25° C. Water (30 mL) was then added, and the resulting mixture was extracted with EtOAc (2×40 mL). The combined organic extract was dried over anhydrous sodium sulfate, filtered, and concentrated. The resulting residue was purified by prep-TLC (CH₂Cl₂/CH₃OH=10/1) to afford tert-butyl 3-[7-[7,8-difluoro-3-(methoxymethoxy)-1-naphthyl]-2-(2,2-dimethoxyethoxy)-8-fluoro-pyrido[4,3-d]pyrimidin-4-yl]-3,8-diazabicyclo[3.2.1]octane-8-carboxylate (360 mg, crude) as a yellow solid. LC/MS (ESI) m/z: 686.0 [M+H]⁺.

Step 11: Preparation of (2S,4R)-1-[(2R*)-2-[3-(7-{2-[(4-{3,8-diazabicyclo[3.2.1]octan-3-yl}-7-(7,8-difluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide

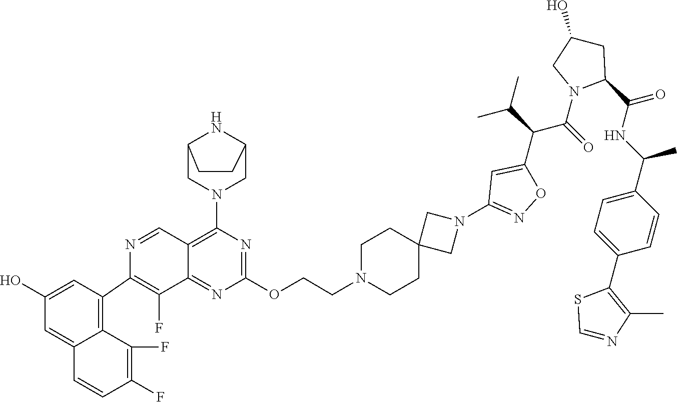 [SMILES: Cc1ncsc1-c1ccc([C@H](C)NC(=O)[C@@H]2C[C@@H](O)CN2C(=O)[C@@H](c2cc(N3CC4(CCN(CCOc5nc(N6CC7CCC(C6)N7)c6cnc(-c7cc(O)cc8ccc(F)c(F)c78)c(F)c6n5)CC4)C3)no2)C(C)C)cc1]

The title compound was prepared in an analogous manner to Compound 16 starting from tert-butyl 3-[7-[7,8-difluoro-3-(methoxymethoxy)-1-naphthyl]-2-(2,2-dimethoxyethoxy)-8-fluoro-pyrido[4,3-d]pyrimidin-4-yl]-3,8-diazabicyclo[3.2.1]octane-8-carboxylate, and purified by prep-HPLC (column: Unisil 3-100 C18 Ultra 150*50 mm*3 um; mobile phase: [10-40% CH₃CN in water (formic acid)]) to afford (2S,4R)-1-[(2R*)-2-[3-(7-{2-[(4-{3,8-diazabicyclo[3.2.1]octan-3-yl}-7-(7,8-difluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (82.7 mg, 0.07 mmol, 71% yield, formic acid salt) as a white solid. LC/MS (ESI) m/z: 1086.5 [M+H]⁺. ¹H NMR (400 MHz, DMSO-d₆) δ 10.45-10.05 (m, 1H), 9.14 (s, 1H), 9.04-8.95 (m, 1H), 8.40 (d, J=7.6 Hz, 1H), 8.15 (s, 1H), 7.81-7.71 (m, 1H), 7.65-7.53 (m, 1H), 7.48-7.42 (m, 2H), 7.41-7.33 (m, 3H), 7.24 (d, J=2.0 Hz, 1H), 5.99-5.67 (m, 1H), 5.20-4.97 (m, 1H), 4.91 (br t, J=7.2 Hz, 1H), 4.61-4.46 (m, 4H), 4.35 (t, J=7.6 Hz, 1H), 4.28 (br s, 1H), 3.87 (br s, 2H), 3.77-3.66 (m, 3H), 3.61-3.49 (m, 6H), 3.41 (br d, J=12.8 Hz, 3H), 2.74-2.68 (m, 3H), 2.45 (s, 3H), 2.24-2.08 (m, 2H), 2.00 (br dd, J=2.8, 7.6 Hz, 1H), 1.83-1.76 (m, 5H), 1.75-1.66 (m, 4H), 1.46-1.35 (m, 3H), 1.00-0.91 (m, 3H), 0.86-0.74 (m, 3H).

Exemplary Synthesis of (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 69)

Step 1: Preparation of O2-benzyl O5-tert-butyl 3,3a,4,6,7,7a-hexahydro-1H-pyrrolo[3,4-c]pyridine-2,5-dicarboxylate

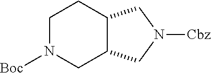

To a solution of tert-butyl 1,2,3,3a,4,6,7,7a-octahydropyrrolo[3,4-c]pyridine-5-carboxylate (3.90 g, 14.84 mmol, 1 eq, hydrochloride) and triethylamine (4.51 g, 44.52 mmol, 6.2 mL, 3 eq) in dichloromethane (40 mL) at 0° C. was added benzyl chloroformate (3.04 g, 17.81 mmol, 2.5 mL, 1.2 eq), and the reaction mixture was stirred at 20° C. for 12 hours. Water (60 mL) was added, and the resulting mixture was extracted with EtOAc (2×50 mL). The combined organic extract was dried over anhydrous sodium sulfate, filtered, and concentrated. The resulting residue was purified by silica gel column chromatography (Petroleum ether/Ethyl acetate=I/O to 3:1) to get O2-benzyl O5-tert-butyl 3,3a,4,6,7,7a-hexahydro-1H-pyrrolo[3,4-c]pyridine-2,5-dicarboxylate (3.42 g, 9.49 mmol, 64% yield) as a colorless gum. ¹H NMR (400 MHz, CDCl₃) δ 7.43-7.29 (m, 5H), 5.21-5.07 (m, 2H), 3.78-3.54 (m, 2H), 3.52-3.43 (m, 2H), 3.40-3.18 (m, 3H), 3.13-2.97 (m, 1H), 2.30 (br d, J=2.8 Hz, 2H), 1.66 (br s, 1H), 1.46 (d, J=2.0 Hz, 10H).

Step 2: Preparation of O2-benzyl O5-tert-butyl(3aS,7aS)-3,3a,4,6,7,7a-hexahydro-1H-pyrrolo[3,4-c]pyridine-2,5-dicarboxylate; O2-benzyl O5-tert-butyl(3aR,7aR)-3,3a,4,6,7,7a-hexahydro-1H-pyrrolo[3,4-c]pyridine-2,5-dicarboxylate

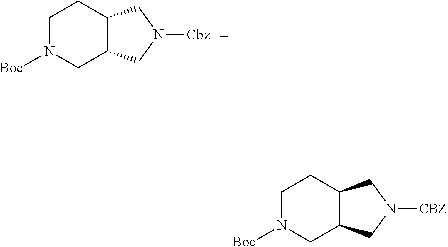

O2-benzyl O5-tert-butyl 3,3a,4,6,7,7a-hexahydro-1H-pyrrolo[3,4-c]pyridine-2,5-dicarboxylate (4 g, 11.10 mmol, 1 eq) was purified by SFC (Column: DAICEL CHIRALPAK AD 250×30 mm, I.D., 10 um; Mobile phase: 35% isopropanol (0.1% NH₄OH) in CO₂; Flow rate: 65 mL/min; Wavelength: 220 nm). O2-benzyl O5-tert-butyl(3aS,7aS)-3,3a,4,6,7,7a-hexahydro-1H-pyrrolo[3,4-c]pyridine-2,5-dicarboxylate (1.7 g, 4.55 mmol, 82% yield, 96.5% purity) (Peak 1, Rt=2.104 min) was obtained as a colorless gum. ¹H NMR (400 MHz, CDCl₃) δ 7.42-7.28 (m, 5H), 5.18-5.09 (m, 2H), 3.79-3.53 (m, 2H), 3.52-3.42 (m, 2H), 3.39-3.16 (m, 3H), 3.14-2.99 (m, 1H), 2.35-2.23 (m, 2H), 1.74-1.61 (m, 1H), 1.46 (d, J=1.6 Hz, 10H). O2-benzyl O5-tert-butyl(3aR,7aR)-3,3a,4,6,7,7a-hexahydro-1H-pyrrolo[3,4-c]pyridine-2,5-dicarboxylate (1.9 g, 4.90 mmol, 88% yield, 93% purity) (Peak 2, Rt=2.351 min) was obtained as a colorless gum. ¹H NMR (400 MHz, CDCl₃) δ 7.42-7.29 (m, 5H), 5.20-5.06 (m, 2H), 3.80-3.55 (m, 2H), 3.54-3.42 (m, 2H), 3.40-3.17 (m, 3H), 3.12-2.99 (m, 1H), 2.39-2.20 (m, 2H), 1.72-1.63 (m, 1H), 1.46 (d, J=1.2 Hz, 10H).

Step 3: Preparation of tert-butyl(3aS,7aR)-1,2,3,3a,4,6,7,7a-octahydropyrrolo[3,4-c]pyridine-5-carboxylate

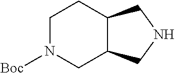 [SMILES: CC(C)(C)OC(=O)N1CC[C@H]2CNC[C@H]2C1]

To a solution of O2-benzyl O5-tert-butyl(3aR,7aR)-3,3a,4,6,7,7a-hexahydro-1H-pyrrolo[3,4-c]pyridine-2,5-dicarboxylate (2.2 g, 6.10 mmol, 1 eq) in tetrahydrofuran (10 mL) and trifluoroethanol (15 mL) were added palladium on activated carbon catalyst (300 mg, 10% purity) under nitrogen, and the resulting mixture was degassed and purged with hydrogen (3×), then stirred at 25° C. under hydrogen (50 Psi) for 16 hours. The mixture was filtered through a pad of celite, and the filtrate was concentrated under vacuum to get tert-butyl(3aS,7aR)-1,2,3,3a,4,6,7,7a-octahydropyrrolo[3,4-c]pyridine-5-carboxylate (1.6 g, crude) as a colorless gum.

Step 4: Preparation of tert-butyl(3aS,7aR)-2-[5-(1-methoxycarbonyl-2-methyl-propyl)isoxazol-3-yl]-3,3a,4,6,7,7a-hexahydro-1H-pyrrolo[3,4-c]pyridine-5-carboxylate

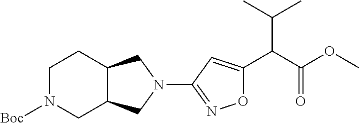 [SMILES: COC(=O)C(c1cc(N2C[C@H]3CN(C(=O)OC(C)(C)C)CC[C@H]3C2)no1)C(C)C]

To a solution of tert-butyl(3aS,7aR)-1,2,3,3a,4,6,7,7a-octahydropyrrolo[3,4-c]pyridine-5-carboxylate (1.5 g, 6.63 mmol, 1 eq) in N,N-dimethylacetamide (15 mL) was added triethylamine (3.35 g, 33.14 mmol, 4.6 mL, 5 eq), and the solution was stirred at 120° C. for 0.5 hours. A solution of methyl 3-methyl-2-[3-(1,1,2,2,3,3,4,4,4-nonafluorobutylsulfonyloxy)isoxazol-5-yl]butanoate (3.19 g, 6.63 mmol, 1 eq) in N,N-dimethylacetamide (15 mL) was then added, and the reaction mixture was stirred at 120° C. for 12 hours. The reaction was cooled to 25° C., diluted with water (60 mL), and extracted with ethyl acetate (2×50 mL). The combined organic extract was washed with brine (4×30 mL), dried over anhydrous sodium sulfate, filtered, and concentrated. The residue was purified by silica gel column chromatography (Petroleum ether/Ethyl acetate=10/1 to 3:1) to afford tert-butyl(3aS,7aR)-2-[5-(1-methoxycarbonyl-2-methyl-propyl)isoxazol-3-yl]-3,3a,4,6,7,7a-hexahydro-1H-pyrrolo[3,4-c]pyridine-5-carboxylate (1.21 g, 2.97 mmol) as a light yellow gum. LC/MS (ESI) m/z: 408.1 [M+H]⁺. ¹H NMR (400 MHz, CDCl₃) δ 5.77 (s, 1H), 3.73 (s, 3H), 3.62 (br d, J=13.6 Hz, 2H), 3.48 (d, J=8.8 Hz, 1H), 3.45-3.34 (m, 3H), 3.27-3.14 (m, 2H), 3.11 (br s, 1H), 2.47-2.28 (m, 3H), 1.77-1.68 (m, 1H), 1.54 (br dd, J=4.4, 8.8 Hz, 1H), 1.46 (s, 9H), 1.01 (d, J=6.8 Hz, 3H), 0.94 (d, J=6.8 Hz, 3H).

Step 5: Preparation of 2-[3-[(3aS,7aR)-5-tert-butoxycarbonyl-3,3a,4,6,7,7a-hexahydro-1H-pyrrolo[3,4-c]pyridin-2-yl]isoxazol-5-yl]-3-methyl-butanoic acid

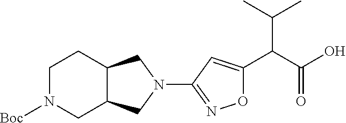 [SMILES: CC(C)C(C(=O)O)c1cc(N2C[C@H]3CN(C(=O)OC(C)(C)C)CC[C@H]3C2)no1]

To a solution of tert-butyl(3aS,7aR)-2-[5-(1-methoxycarbonyl-2-methyl-propyl)isoxazol-3-yl]-3,3a,4,6,7,7a-hexahydro-1H-pyrrolo[3,4-c]pyridine-5-carboxylate (1.29 g, 3.17 mmol, 1 eq) in tetrahydrofuran (4 mL), methanol (4 mL) and water (4 mL) was added lithium hydroxide (664 mg, 15.83 mmol, 5 eq), and the reaction mixture was stirred at 20° C. for 2 hours. The pH was adjusted to 6 by addition with aqueous HCl (1 M) at 0° C. The resulting solution was extracted with ethyl acetate (6×30 mL), and the combined organic extract was dried over anhydrous sodium sulfate, filtered, and concentrated to afford 2-[3-[(3aS,7aR)-5-tert-butoxycarbonyl-3,3a,4,6,7,7a-hexahydro-1H-pyrrolo[3,4-c]pyridin-2-yl]isoxazol-5-yl]-3-methyl-butanoic acid (1.1 g, 2.80 mmol, 88% yield) as a light yellow gum. LC/MS (ESI) m/z: 394.2 [M+H]⁺. ¹H NMR (400 MHz, CDCl₃) δ 5.79 (s, 1H), 3.76-3.54 (m, 2H), 3.49-3.31 (m, 4H), 3.26-3.03 (m, 4H), 2.43-2.29 (m, 3H), 1.72 (br d, J=12.8 Hz, 1H), 1.45 (s, 9H), 1.04 (br d, J=6.8 Hz, 3H), 0.96-0.90 (m, 3H).

Step 6: Preparation of tert-butyl(3aS,7aR)-2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridine-5-carboxylate

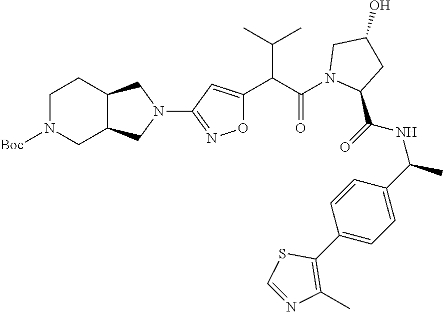 [SMILES: Cc1ncsc1-c1ccc([C@H](C)NC(=O)[C@@H]2C[C@@H](O)CN2C(=O)C(c2cc(N3C[C@H]4CN(C(=O)OC(C)(C)C)CC[C@H]4C3)no2)C(C)C)cc1]

To a mixture of 2-[3-[(3aS,7aR)-5-tert-butoxycarbonyl-3,3a,4,6,7,7a-hexahydro-1H-pyrrolo[3,4-c]pyridin-2-yl]isoxazol-5-yl]-3-methyl-butanoic acid (1.1 g, 2.80 mmol, 1 eq) and (2S,4R)-4-hydroxy-N-[(1S)-1-[4-(4-methylthiazol-5-yl)phenyl]ethyl]pyrrolidine-2-carboxamide (1.08 g, 2.94 mmol, 1.05 eq, hydrochloride) in N,N-dimethylformamide (15 mL) at 0° C. was added N,N-diisopropylethylamine (1.09 g, 8.40 mmol, 1.5 mL, 3 eq) followed by O-(7-azabenzotriazol-1-yl)-N,N,N′,N′-tetramethyluronium Hexafluorophosphate (1.28 g, 3.36 mmol, 1.2 eq), and the reaction mixture was stirred at 20° C. for 1 hour. The mixture was diluted with ethyl acetate (250 mL) and washed with brine (4×30 mL), dried over anhydrous sodium sulfate, filtered, and concentrated. The resulting residue was purified by silica gel column chromatography (Petroleum ether/Ethyl acetate=10/1 to Ethyl acetate/Methanol=12:1) to afford tert-butyl(3aS,7aR)-2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridine-5-carboxylate (1.6 g, 2.15 mmol, 77% yield) as a light yellow solid. LC/MS (ESI) m/z: 707.3 [M+H]⁺. ¹H NMR (400 MHz, CDCl₃) δ 8.68 (d, J=1.6 Hz, 1H), 7.41-7.30 (m, 4H), 5.76 (d, J=9.2 Hz, 1H), 5.14-4.93 (m, 1H), 4.82-4.51 (m, 2H), 3.81-3.52 (m, 5H), 3.49-3.28 (m, 4H), 3.20-3.02 (m, 3H), 2.53 (d, J=4.0 Hz, 4H), 2.47-2.36 (m, 3H), 2.02-1.91 (m, 1H), 1.70 (br dd, J=3.6, 14.0 Hz, 1H), 1.53-1.33 (m, 14H), 1.04 (d, J=6.4 Hz, 3H), 0.94 (d, J=6.8 Hz, 3H).

Step 7: Preparation of tert-butyl(3aS,7aR)-2-[5-[(1S)-1-[(2S,4R)-4-hydroxy-2-[[(1S)-1-[4 (4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridine-5-carboxylate and tert-butyl(3aS,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridine-5-carboxylate

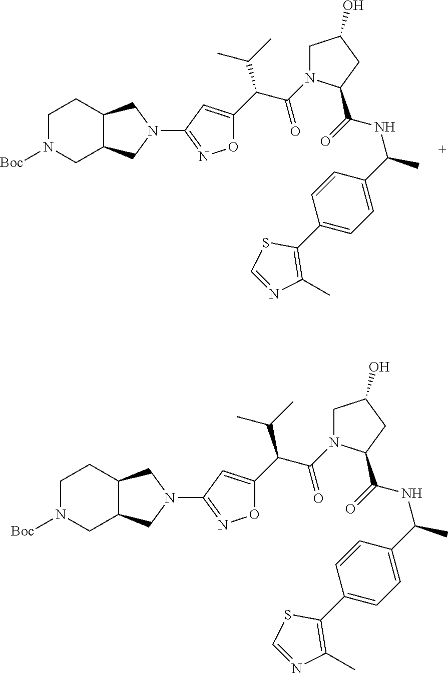 [SMILES: Cc1ncsc1-c1ccc([C@H](C)NC(=O)[C@@H]2C[C@@H](O)CN2C(=O)[C@H](c2cc(N3C[C@H]4CN(C(=O)OC(C)(C)C)CC[C@H]4C3)no2)C(C)C)cc1 ; Cc1ncsc1-c1ccc([C@H](C)NC(=O)[C@@H]2C[C@@H](O)CN2C(=O)[C@@H](c2cc(N3C[C@H]4CN(C(=O)OC(C)(C)C)CC[C@H]4C3)no2)C(C)C)cc1]

tert-Butyl (3aS,7aR)-2-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridine-5-carboxylate (1.6 g, 2.15 mmol, 95% purity, 1 eq) was purified by SFC (Column: DAICEL CHIRALPAK IG 250×30 mm, I.D., 10 um; Mobile phase: 60% acetonitrile/isopropanol (0.1% NH₄OH) in CO₂; Flow rate: 70 mL/min; Wavelength: 220 nm).

tert-Butyl (3aS,7aR)-2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridine-5-carboxylate (740 mg, 1.04 mmol, 96% yield, 99% purity) (Peak 1, Rt=0.662 min) was obtained as a light yellow solid. ¹H NMR (400 MHz, CDCl₃) δ 8.67 (s, 1H), 7.39 (d, J=8.0 Hz, 2H), 7.36-7.31 (m, 2H), 5.75 (s, 1H), 4.97 (t, J=7.2 Hz, 1H), 4.80 (dd, J=4.0, 8.4 Hz, 1H), 4.68-4.57 (m, 1H), 3.78-3.71 (m, 1H), 3.66-3.51 (m, 3H), 3.41-3.29 (m, 3H), 3.24-3.04 (m, 3H), 2.90 (d, J=4.0 Hz, 1H), 2.65-2.57 (m, 1H), 2.53 (s, 3H), 2.50-2.34 (m, 3H), 2.05-1.94 (m, 1H), 1.66 (br s, 2H), 1.56-1.33 (m, 14H), 1.05 (d, J=6.4 Hz, 3H), 0.94 (d, J=6.8 Hz, 3H), 0.87-0.82 (m, 1H).

tert-Butyl (3aS,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridine-5-carboxylate (750 mg, 1.02 mmol, 95% yield, 96% purity) (Peak 2, Rt=1.269 min) was obtained as a light yellow solid. ¹H NMR (400 MHz, CDCl₃) δ 8.67 (s, 1H), 7.44-7.33 (m, 4H), 5.75 (s, 1H), 5.07 (t, J=7.2 Hz, 1H), 4.69-4.51 (m, 2H), 3.77 (br dd, J=4.8, 10.4 Hz, 1H), 3.68-3.53 (m, 3H), 3.50 (d, J=10.0 Hz, 1H), 3.39-3.30 (m, 3H), 3.21-3.03 (m, 3H), 2.53 (s, 3H), 2.48-2.30 (m, 4H), 2.01-1.89 (m, 1H), 1.78-1.65 (m, 1H), 1.50 (br d, J=6.8 Hz, 5H), 1.45 (s, 9H), 1.04 (d, J=6.4 Hz, 3H), 0.93 (d, J=6.8 Hz, 3H).

Step 8: Preparation of (2S,4R)-1-[(2R)-2-[3-[(3aR,7aR)-1,3,3a,4,5,6,7,7a-octahydropyrrolo[3,4-c]pyridin-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

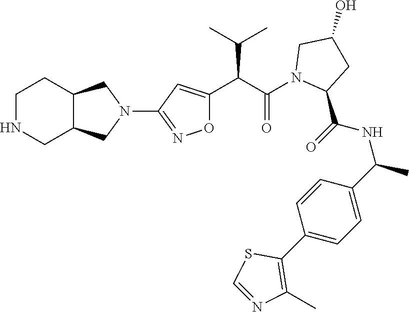 [SMILES: Cc1ncsc1-c1ccc([C@H](C)NC(=O)[C@@H]2C[C@@H](O)CN2C(=O)[C@@H](c2cc(N3C[C@H]4CNCC[C@H]4C3)no2)C(C)C)cc1]

To a solution of tert-butyl(3aS,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridine-5-carboxylate (120 mg, 0.16 mmol, 96.5% purity, 1 eq) in dichloromethane (5 mL) was added trifluoroacetic acid (1.54 g, 13.51 mmol, 1 mL, 82.45 eq), and the reaction mixture was stirred at 25° C. for 1 hour. The mixture was concentrated under vacuum to afford (2S,4R)-1-[(2R)-2-[3-[(3aR,7aR)-1,3,3a,4,5,6,7,7a-octahydropyrrolo[3,4-c]pyridin-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (120 mg, crude, trifluoroacetate) as a light yellow solid.

Step 9: Preparation of (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

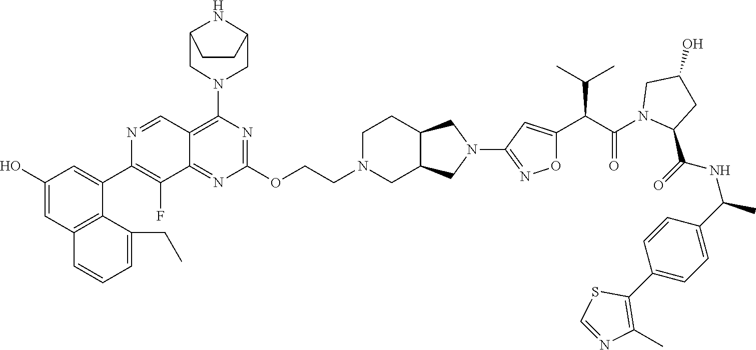 [SMILES: CCc1cccc2cc(O)cc(-c3ncc4c(N5CC6CCC(C5)N6)nc(OCCN5CC[C@H]6CN(c7cc([C@H](C(=O)N8C[C@H](O)C[C@H]8C(=O)N[C@@H](C)c8ccc(-c9scnc9C)cc8)C(C)C)on7)C[C@H]6C5)nc4c3F)c12]

The title compound was prepared in an analogous manner to Compound 16 starting from (2S,4R)-1-[(2R)-2-[3-[(3aR,7aR)-1,3,3a,4,5,6,7,7a-octahydropyrrolo[3,4-c]pyridin-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate, and purified by prep-HPLC (column: Unisil 3-100 C18 Ultra 150*50 mm*3 um; mobile phase: [12-42% CH₃CN in water (FA)]) to afford (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (53.4 mg, 0.05 mmol, 68% yield, 98.1% purity, formic acid salt) as a white solid. LC/MS (ESI) m/z: 1078.3 [M+H]⁺. ¹H NMR (400 MHz, DMSO-d₆) δ 9.12 (d, J=2.0 Hz, 1H), 8.99 (s, 1H), 8.39 (br d, J=7.6 Hz, 1H), 8.16 (s, 1H), 7.67 (d, J=8.8 Hz, 1H), 7.50-7.41 (m, 2H), 7.39-7.33 (m, 3H), 7.29 (d, J=2.4 Hz, 1H), 7.13 (d, J=6.8 Hz, 1H), 6.97 (t, J=2.8 Hz, 1H), 5.88-5.66 (m, 1H), 4.91 (br t, J=7.2 Hz, 1H), 4.62-4.42 (m, 4H), 4.36 (s, 1H), 4.28 (br s, 1H), 3.98-3.80 (m, 3H), 3.80-3.66 (m, 3H), 3.53 (br d, J=2.0 Hz, 1H), 3.41 (br d, J=10.4 Hz, 2H), 3.34-3.25 (m, 2H), 3.23-3.01 (m, 4H), 2.76-2.64 (m, 3H), 2.47-2.44 (m, 3H), 2.35 (br s, 1H), 2.30-2.10 (m, 5H), 2.01 (br t, J=9.2 Hz, 1H), 1.86-1.64 (m, 6H), 1.50-1.32 (m, 4H), 1.02-0.90 (m, 3H), 0.87-0.73 (m, 6H).

Exemplary Synthesis of (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 70)

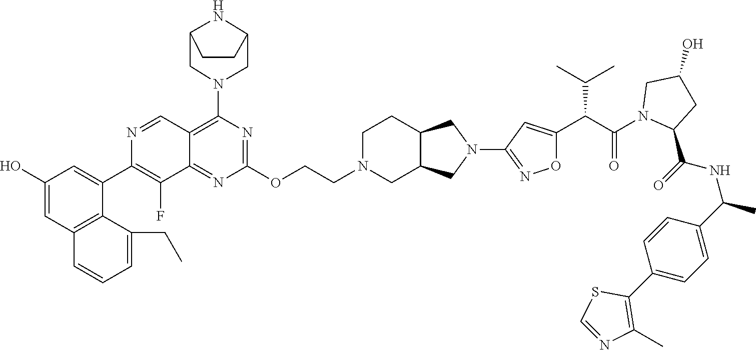 [SMILES: CCc1cccc2cc(O)cc(-c3ncc4c(N5CC6CCC(C5)N6)nc(OCCN5CC[C@H]6CN(c7cc([C@@H](C(=O)N8C[C@H](O)C[C@H]8C(=O)N[C@@H](C)c8ccc(-c9scnc9C)cc8)C(C)C)on7)C[C@H]6C5)nc4c3F)c12]

The title compound was prepared in an analogous manner to Compound 69 starting from (2S,4R)-1-[(2S)-2-[3-[(3aR,7aR)-1,3,3a,4,5,6,7,7a-octahydropyrrolo[3,4-c]pyridin-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate, and purified by prep-HPLC (column: Waters Xbridge 150*25 mm*5 um; mobile phase: [38-68% CH₃CN in water (NH₄HCO₃)]) to afford (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (30.3 mg, 0.03 mmol, 48% yield, 98% purity) as a white solid. LC/MS (ESI) m/z: 1078.4 [M+H]⁺. ¹H NMR: (400 MHz, DMSO-d₆) δ 9.13-9.02 (m, 1H), 9.01-8.89 (m, 1H), 8.23-8.12 (m, 1H), 7.65 (d, J=8.1 Hz, 1H), 7.49-7.43 (m, 1H), 7.42-7.38 (m, 1H), 7.38-7.33 (m, 2H), 7.30-7.25 (m, 2H), 7.10 (br d, J=6.8 Hz, 1H), 6.95 (d, J=2.0 Hz, 1H), 5.87-5.79 (m, 1H), 5.06-4.79 (m, 1H), 4.55-4.38 (m, 5H), 4.30-4.17 (m, 1H), 3.77-3.60 (m, 5H), 3.52 (br s, 2H), 3.45-3.39 (m, 2H), 3.22-2.99 (m, 5H), 2.45 (br s, 2H), 2.42 (s, 3H), 2.34-2.30 (m, 2H), 2.29-2.14 (m, 6H), 2.05-1.95 (m, 1H), 1.78-1.57 (m, 6H), 1.44 (br d, J=6.8 Hz, 2H), 1.32 (d, J=7.2 Hz, 2H), 0.98-0.91 (m, 2H), 0.82-0.76 (m, 6H), 0.73 (br d, J=6.8 Hz, 1H).

Exemplary Synthesis of (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 71)

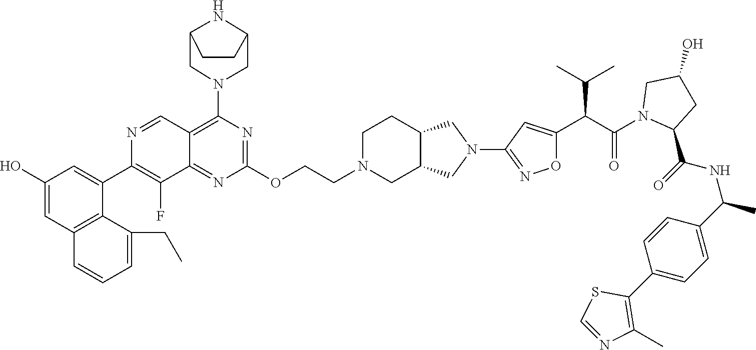 [SMILES: CCc1cccc2cc(O)cc(-c3ncc4c(N5CC6CCC(C5)N6)nc(OCCN5CC[C@@H]6CN(c7cc([C@H](C(=O)N8C[C@H](O)C[C@H]8C(=O)N[C@@H](C)c8ccc(-c9scnc9C)cc8)C(C)C)on7)C[C@@H]6C5)nc4c3F)c12]

The title compound was prepared in an analogous manner to Compound 69 starting from O2-benzyl O5-tert-butyl(3aS,7aS)-3,3a,4,6,7,7a-hexahydro-1H-pyrrolo[3,4-c]pyridine-2,5-dicarboxylate, and purified by prep-HPLC (column: Unisil 3-100 C18 Ultra 150*50 mm*3 um; mobile phase: [12-42% CH₃CN in water (FA)]) to afford (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (41.6 mg, 0.04 mmol, 71% yield, 99.7% purity) as a white solid. LC/MS (ESI) m/z: 1078.3 [M+H]⁺. ¹H NMR (400 MHz, DMSO-d₆) δ 9.97-9.81 (m, 1H), 9.11 (d, J=2.8 Hz, 1H), 8.97 (s, 1H), 8.38 (dd, J=3.2, 7.6 Hz, 1H), 7.66 (d, J=7.6 Hz, 1H), 7.51-7.40 (m, 2H), 7.38-7.31 (m, 3H), 7.27 (d, J=2.4 Hz, 1H), 7.17-7.07 (m, 1H), 6.95 (d, J=2.4 Hz, 1H), 5.85-5.62 (m, 1H), 4.90 (br t, J=7.2 Hz, 1H), 4.72-4.39 (m, 4H), 4.35 (dt, J=1.6, 7.6 Hz, 1H), 4.26 (br d, J=1.6 Hz, 1H), 3.81 (br d, J=12.0 Hz, 2H), 3.76-3.62 (m, 3H), 3.56-3.49 (m, 1H), 3.45-3.35 (m, 3H), 3.28-3.14 (m, 4H), 3.12-3.04 (m, 2H), 2.73-2.64 (m, 3H), 2.45-2.43 (m, 3H), 2.34 (br s, 1H), 2.29-2.12 (m, 5H), 2.04-1.94 (m, 1H), 1.82-1.62 (m, 6H), 1.49-1.33 (m, 4H), 0.93 (br d, J=5.6 Hz, 3H), 0.85-0.73 (m, 6H).

Exemplary Synthesis of (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 72)

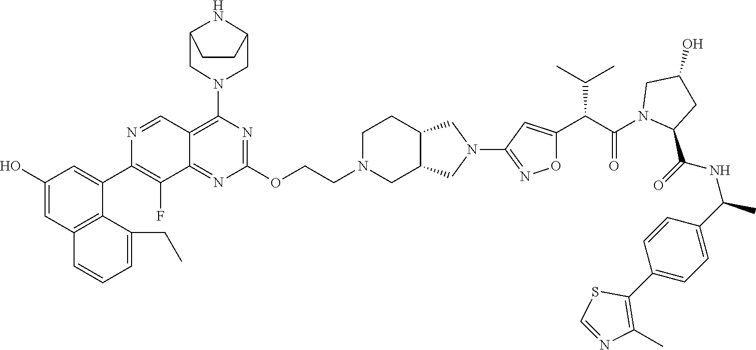 [SMILES: CCc1cccc2cc(O)cc(-c3ncc4c(N5CC6CCC(C5)N6)nc(OCCN5CC[C@@H]6CN(c7cc([C@@H](C(=O)N8C[C@H](O)C[C@H]8C(=O)N[C@@H](C)c8ccc(-c9scnc9C)cc8)C(C)C)on7)C[C@@H]6C5)nc4c3F)c12]

The title compound was prepared in an analogous manner to Compound 70 starting from O2-benzyl O5-tert-butyl(3aS,7aS)-3,3a,4,6,7,7a-hexahydro-1H-pyrrolo[3,4-c]pyridine-2,5-dicarboxylate, and purified by prep-HPLC (column: Waters Xbridge 150*25 mm*5 um; mobile phase: [38-68% CH₃CN in water (NH₄HCO₃)]) followed by prep-HPLC (column: Phenomenex Synergi C18 150*25 mm*10 um; mobile phase: [10-40% CH₃CN in water (FA)]) to afford (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (26.7 mg, 0.02 mmol, 40% yield, 98.9% purity, formic acid salt) as a white solid. LC/MS (ESI) m/z: 1078.4 [M+H]⁺. ¹H NMR (400 MHz, DMSO-d₆) δ 9.08 (d, J=2.8 Hz, 1H), 9.01-8.93 (m, 1H), 8.22 (s, 1H), 8.15 (br d, J=7.6 Hz, 1H), 7.66 (d, J=8.4 Hz, 1H), 7.52-7.42 (m, 1H), 7.41-7.33 (m, 3H), 7.32-7.27 (m, 2H), 7.11 (d, J=7.2 Hz, 1H), 6.97 (t, J=3.6 Hz, 1H), 5.89-5.83 (m, 1H), 4.87 (s, 1H), 4.54-4.38 (m, 5H), 4.30-4.23 (m, 1H), 3.69-3.63 (m, 2H), 3.59 (br d, J=7.2 Hz, 3H), 3.46-3.39 (m, 2H), 3.31-3.22 (m, 3H), 3.19-2.98 (m, 4H), 2.67 (br d, J=2.0 Hz, 1H), 2.46 (br s, 2H), 2.44 (s, 3H), 2.35-2.32 (m, 1H), 2.31-2.13 (m, 6H), 2.07-1.98 (m, 1H), 1.85-1.73 (m, 1H), 1.71-1.56 (m, 5H), 1.45 (br d, J=6.8 Hz, 2H), 1.33 (d, J=6.8 Hz, 2H), 0.95 (d, J=6.8 Hz, 2H), 0.84-0.77 (m, 6H), 0.75 (br d, J=6.8 Hz, 1H).

Exemplary Synthesis of (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,2-c]pyridin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 73)

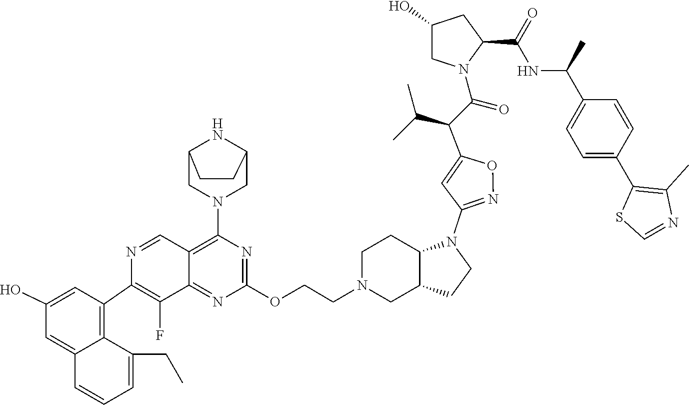 [SMILES: CCc1cccc2cc(O)cc(-c3ncc4c(N5CC6CCC(C5)N6)nc(OCCN5CC[C@H]6[C@H](CCN6c6cc([C@H](C(=O)N7C[C@H](O)C[C@H]7C(=O)N[C@@H](C)c7ccc(-c8scnc8C)cc7)C(C)C)on6)C5)nc4c3F)c12]

The title compound was made in an analogous manner to Compound 69 starting from tert-butyl 1,2,3,3a,4,6,7,7a-octahydropyrrolo[3,2-c]pyridine-5-carboxylate, and purified by prep-HPLC (column: Unisil 3-100 C18 Ultra 150*50 mm*3 um; mobile phase: [13-43% CH₃CN in water (FA)]) to afford (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,2-c]pyridin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (15.6 mg, 0.14 mmol, 20% yield, 97% purity) as a white solid. LC/MS (ESI) m/z: 1078.5 [M+H]⁺. ¹H NMR (400 MHz, DMSO-d₆) δ 10.08-9.77 (m, 1H), 9.08 (s, 1H), 8.98 (s, 1H), 8.46-8.34 (m, 1H), 7.73-7.62 (m, 1H), 7.51-7.23 (m, 6H), 7.20-7.09 (m, 1H), 7.02-6.92 (m, 1H), 5.95-5.84 (m, 1H), 5.25-5.00 (m, 1H), 4.97-4.87 (m, 1H), 4.60-4.41 (m, 4H), 4.40-4.19 (m, 2H), 3.77-3.45 (m, 9H), 3.25-3.15 (m, 2H), 2.80-2.70 (m, 2H), 2.65-2.63 (m, 1H), 2.45 (s, 3H), 2.31-2.15 (m, 6H), 2.05-1.90 (m, 3H), 1.88-1.73 (m, 4H), 1.71-1.60 (m, 4H), 1.46-1.34 (m, 3H), 1.00-0.91 (m, 3H), 0.86-0.76 (m, 6H).

Exemplary Synthesis (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,2-c]pyridin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 74)

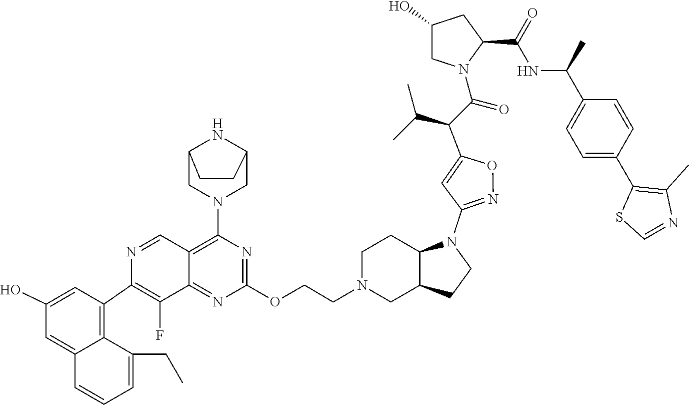 [SMILES: CCc1cccc2cc(O)cc(-c3ncc4c(N5CC6CCC(C5)N6)nc(OCCN5CC[C@@H]6[C@@H](CCN6c6cc([C@H](C(=O)N7C[C@H](O)C[C@H]7C(=O)N[C@@H](C)c7ccc(-c8scnc8C)cc7)C(C)C)on6)C5)nc4c3F)c12]

The title compound was made in an analogous manner to Compound 69 starting from tert-butyl 1,2,3,3a,4,6,7,7a-octahydropyrrolo[3,2-c]pyridine-5-carboxylate, and purified by prep-HPLC (column: Unisil 3-100 C18 Ultra 150*50 mm*3 um; mobile phase: [13-43% CH₃CN in water (FA)]) to afford (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,2-c]pyridin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (75.1 mg, 0.06 mmol, 67% yield, 98% purity) as a white solid. LC/MS (ESI) m/z: 1078.5 [M+H⁺. ¹H NMR (400 MHz, DMSO-d₆) δ 10.08-9.67 (m, 1H), 9.12 (s, 1H), 8.98 (s, 1H), 8.81-8.34 (m, 1H), 7.74-7.60 (m, 1H), 7.47-7.28 (m, 6H), 7.18-7.07 (m, 1H), 7.01-6.93 (m, 1H), 5.97-5.76 (m, 1H), 5.30-4.98 (m, 1H), 4.96-4.86 (m, 1H), 4.62-4.47 (m, 4H), 4.41-4.22 (m, 2H), 3.99-3.90 (m, 2H), 3.83-3.66 (m, 4H), 3.60-3.52 (m, 3H), 3.22-3.13 (m, 2H), 2.78-2.69 (m, 3H), 2.45 (s, 3H), 2.33-2.09 (m, 6H), 2.09-1.85 (m, 4H), 1.85-1.71 (m, 6H), 1.67-1.51 (m, 1H), 1.48-1.34 (m, 3H), 1.00-0.91 (m, 3H), 0.88-0.76 (m, 6H).

Exemplary Synthesis of (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,2-c]pyridin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 75)

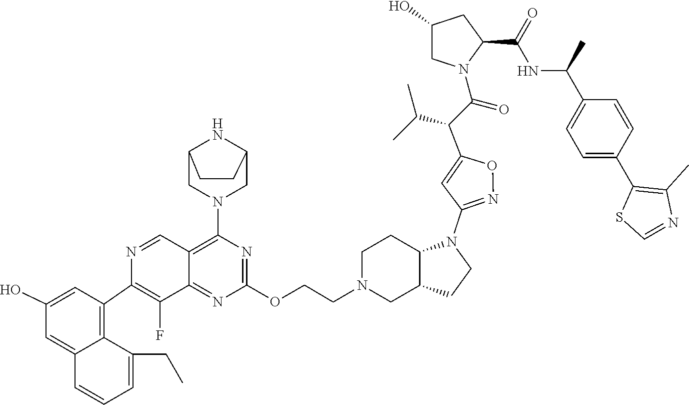 [SMILES: CCc1cccc2cc(O)cc(-c3ncc4c(N5CC6CCC(C5)N6)nc(OCCN5CC[C@H]6[C@H](CCN6c6cc([C@@H](C(=O)N7C[C@H](O)C[C@H]7C(=O)N[C@@H](C)c7ccc(-c8scnc8C)cc7)C(C)C)on6)C5)nc4c3F)c12]

The title compound was made in an analogous manner to Compound 70 starting from tert-butyl 1,2,3,3a,4,6,7,7a-octahydropyrrolo[3,2-c]pyridine-5-carboxylate, and purified prep-HPLC (column: Unisil 3-100 C18 Ultra 150*50 mm*3 um; mobile phase: [13-43% CH₃CN in to afford (2S,4R)-1-[(2R*)-2-{3-[(3aR**,7aR***)-5-{2-[(4-{3,8-water (FA)])diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,2-c]pyridin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (41.2 mg, 0.03 mmol, 44% yield, 99% purity) as a white solid. LC/MS (ESI) m/z: 1078.3 [M+H]⁺. ¹H NMR (400 MHz, DMSO-d₆) δ 9.97-9.83 (m, 1H), 9.11 (s, 1H), 9.02-8.92 (m, 1H), 8.27-8.11 (m, 1H), 7.73-7.62 (m, 1H), 7.52-7.25 (m, 6H), 7.17-7.04 (m, 1H), 7.02-6.92 (m, 1H), 5.98-5.88 (m, 1H), 5.19-4.92 (m, 1H), 4.89-4.78 (m, 1H), 4.63-4.36 (m, 5H), 4.32-4.20 (m, 1H), 3.92-3.80 (m, 2H), 3.77-3.60 (m, 3H), 3.57-3.41 (m, 4H), 3.23-3.08 (m, 2H), 2.77-2.64 (m, 3H), 2.43 (s, 3H), 2.31-2.13 (m, 5H), 2.12-1.87 (m, 4H), 1.86-1.65 (m, 7H), 1.59-1.47 (m, 1H), 1.46-1.31 (m, 3H), 1.00-0.92 (m, 2H), 0.89-0.72 (m, 7H).

Exemplary Synthesis of (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,2-c]pyridin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 76)

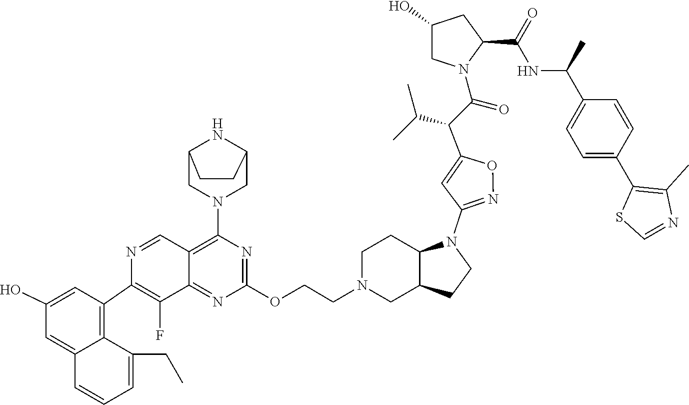 [SMILES: CCc1cccc2cc(O)cc(-c3ncc4c(N5CC6CCC(C5)N6)nc(OCCN5CC[C@@H]6[C@@H](CCN6c6cc([C@@H](C(=O)N7C[C@H](O)C[C@H]7C(=O)N[C@@H](C)c7ccc(-c8scnc8C)cc7)C(C)C)on6)C5)nc4c3F)c12]

The title compound was made in an analogous manner to Compound 70 starting from tert-butyl 1,2,3,3a,4,6,7,7a-octahydropyrrolo[3,2-c]pyridine-5-carboxylate, and purified by prep-HPLC (column: Unisil 3-100 C18 Ultra 150*50 mm*3 um; mobile phase: [12-42% CH₃CN in water (FA)]) to afford (2S,4R)-1-[(2R*)-2-{3-[(3aR**,7aR***)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydro-1H-pyrrolo[3,2-c]pyridin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (26.7 mg, 0.24 mmol, 28% yield, 99% purity) as a white solid. LC/MS (ESI) m/z: 1078.7 [M+H]⁺. ¹H NMR (400 MHz, DMSO-d₆) δ 10.08-9.63 (m, 1H), 9.09 (s, 1H), 9.01-8.90 (m, 1H), 8.22-8.14 (m, 1H), 7.72-7.63 (m, 1H), 7.51-7.25 (m, 6H), 7.15-7.06 (m, 1H), 7.01-6.93 (m, 1H), 6.00-5.86 (m, 1H), 5.25-4.95 (m, 1H), 4.91-4.79 (m, 1H), 4.57-4.35 (m, 5H), 4.32-4.22 (m, 1H), 3.76-3.61 (m, 5H), 3.57-3.43 (m, 4H), 3.21-3.07 (m, 2H), 2.77-2.64 (m, 3H), 2.44 (s, 3H), 2.31-2.14 (m, 5H), 2.13-1.88 (m, 4H), 1.87-1.75 (m, 3H), 1.74-1.63 (m, 4H), 1.59-1.49 (m, 1H), 1.48-1.32 (m, 3H), 0.99-0.92 (m, 2H), 0.88-0.72 (m, 7H).

Exemplary Synthesis of (2S,4R)-1-[(2R*)-2-{3-[(3aR,6aR)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydropyrrolo[3,4-c]pyrrol-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 77)

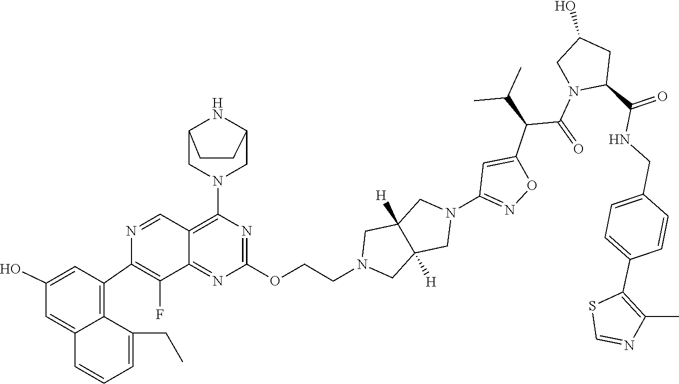 [SMILES: CCc1cccc2cc(O)cc(-c3ncc4c(N5CC6CCC(C5)N6)nc(OCCN5C[C@@H]6CN(c7cc([C@H](C(=O)N8C[C@H](O)C[C@H]8C(=O)NCc8ccc(-c9scnc9C)cc8)C(C)C)on7)C[C@H]6C5)nc4c3F)c12]

The title compound was made in an analogous manner to Compound 69 starting from tert-butyl(3aS,6aS)-2,3,3a,4,6,6a-hexahydro-1H-pyrrolo[3,4-c]pyrrole-5-carboxylate, and purified by prep-HPLC (column: Unisil 3-100 C18 Ultra 150*50 mm*3 um; mobile phase: [11-41% CH₃CN in water (formic acid)]) to afford (2S,4R)-1-[(2R*)-2-{3-[(3aR,6aR)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydropyrrolo[3,4-c]pyrrol-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (29.6 mg, 0.02 mmol, 65% yield, formic acid salt) as a white solid. LC/MS (ESI) m/z: 928.0 [M+H]⁺. ¹H NMR (400 MHz, DMSO-d₆) δ 10.17-9.56 (m, 1H), 9.10 (s, 1H), 8.98 (s, 1H), 8.83-8.32 (m, 1H), 8.22-8.12 (m, 1H), 7.72-7.61 (m, 1H), 7.51-7.24 (m, 6H), 7.17-7.09 (m, 1H), 7.01-6.93 (m, 1H), 5.93-5.71 (m, 1H), 5.20-4.83 (m, 2H), 4.59-4.41 (m, 4H), 4.39-4.24 (m, 2H), 3.80-3.62 (m, 8H), 3.13-3.08 (m, 2H), 3.05-2.87 (m, 5H), 2.53-2.52 (m, 2H), 2.45 (s, 3H), 2.30-2.11 (m, 5H), 2.06-1.85 (m, 2H), 1.84-1.64 (m, 5H), 1.48-1.35 (m, 3H), 1.00-0.90 (m, 3H), 0.88-0.73 (m, 6H).

Exemplary Synthesis of (2S,4R)-1-[(2R*)-2-{3-[(3aR,6aR)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydropyrrolo[3,4-c]pyrrol-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 78)

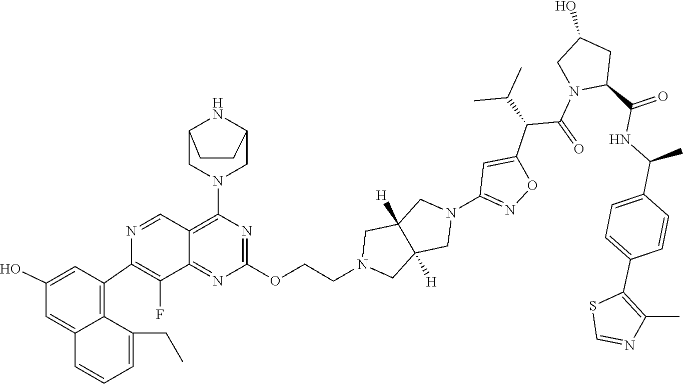 [SMILES: CCc1cccc2cc(O)cc(-c3ncc4c(N5CC6CCC(C5)N6)nc(OCCN5C[C@@H]6CN(c7cc([C@@H](C(=O)N8C[C@H](O)C[C@H]8C(=O)N[C@@H](C)c8ccc(-c9scnc9C)cc8)C(C)C)on7)C[C@H]6C5)nc4c3F)c12]

The title compound was made in an analogous manner to Compound 70 starting from tert-butyl(3aS,6aS)-2,3,3a,4,6,6a-hexahydro-1H-pyrrolo[3,4-c]pyrrole-5-carboxylate, and purified by prep-HPLC (column: Unisil 3-100 C18 Ultra 150*50 mm*3 um; mobile phase: [10-40% CH₃CN in water (formic acid)) to afford (2S,4R)-1-[(2R*)-2-{3-[(3aR,6aR)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydropyrrolo[3,4-c]pyrrol-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (13.4 mg, 0.01 mmol, 28% yield, formic acid salt) as a white solid. LC/MS (ESI) m/z: 1065.1 [M+H]⁺. ¹H NMR (400 MHz, DMSO-d₆) δ 10.05-9.76 (m, 1H), 9.08 (s, 1H), 9.03-8.96 (m, 1H), 8.93-8.13 (m, 1H), 7.72-7.62 (m, 1H), 7.47-7.27 (m, 6H), 7.16-7.07 (m, 1H), 7.01-6.92 (m, 1H), 5.94-5.81 (m, 1H), 5.14-4.83 (m, 2H), 4.55-4.37 (m, 5H), 4.32-4.21 (m, 1H), 3.68-3.51 (m, 8H), 3.11-3.04 (m, 2H), 2.99-2.86 (m, 5H), 2.62-2.60 (m, 2H), 2.43 (s, 3H), 2.29-2.18 (m, 5H), 2.09-1.87 (m, 2H), 1.82-1.61 (m, 5H), 1.48-1.33 (m, 3H), 0.98-0.93 (m, 2H), 0.87-0.73 (m, 7H).

Exemplary Synthesis of (2S,4R)-1-[(2R*)-2-{3-[(3aR,6aS)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydropyrrolo[3,4-c]pyrrol-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 79)

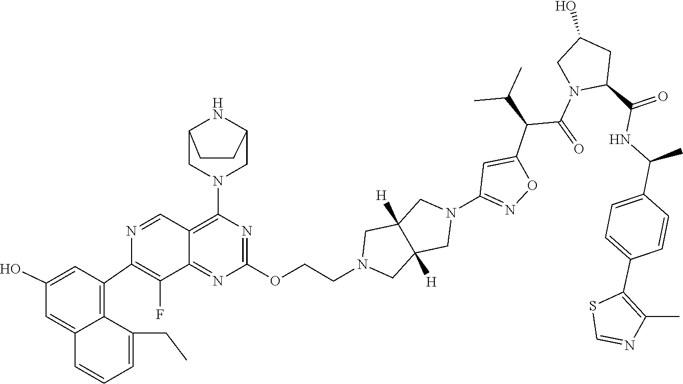 [SMILES: CCc1cccc2cc(O)cc(-c3ncc4c(N5CC6CCC(C5)N6)nc(OCCN5C[C@@H]6CN(c7cc([C@H](C(=O)N8C[C@H](O)C[C@H]8C(=O)N[C@@H](C)c8ccc(-c9scnc9C)cc8)C(C)C)on7)C[C@@H]6C5)nc4c3F)c12]

The title compound was made in an analogous manner to Compound 69 starting from tert-butyl(3aS,6aR)-2,3,3a,4,6,6a-hexahydro-1H-pyrrolo[3,4-c]pyrrole-5-carboxylate, and purified by prep-HPLC (column: Unisil 3-100 C18 Ultra 150*50 mm*3 um; mobile phase: [12-42% CH₃CN in water (formic acid)]) to afford (2S,4R)-1-[(2R*)-2-{3-[(3aR,6aS)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydropyrrolo[3,4-c]pyrrol-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (41.3 mg, 0.038 mmol, 46% yield, formic acid salt) as a white solid. LC/MS (ESI) m/z: 1064.3 [M+H]⁺. ¹H NMR (400 MHz, DMSO-d₆) δ 9.14-9.06 (m, 1H), 8.99 (s, 1H), 8.47-8.36 (m, 1H), 8.20 (s, 1H), 7.67 (d, J=8.1 Hz, 1H), 7.48-7.41 (m, 2H), 7.40-7.33 (m, 3H), 7.13 (d, J=6.7 Hz, 1H), 6.98 (d, J=1.8 Hz, 1H), 5.97 (s, 1H), 4.92 (quin, J=6.9 Hz, 1H), 4.55-4.39 (m, 4H), 4.38-4.33 (m, 1H), 4.28 (br s, 1H), 3.78-3.53 (m, 7H), 3.46-3.37 (m, 1H), 3.29 (br s, 2H), 3.07-2.98 (m, 2H), 2.79 (br t, J=5.5 Hz, 6H), 2.47-2.42 (m, 6H), 2.31-2.19 (m, 3H), 2.06-1.96 (m, 1H), 1.81-1.63 (m, 5H), 1.38 (d, J=7.0 Hz, 3H), 1.01-0.91 (m, 3H), 0.84-0.75 (m, 6H).

Exemplary Synthesis of (2S,4R)-1-[(2R*)-2-{3-[(3aR,6aS)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydropyrrolo[3,4-c]pyrrol-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 80)

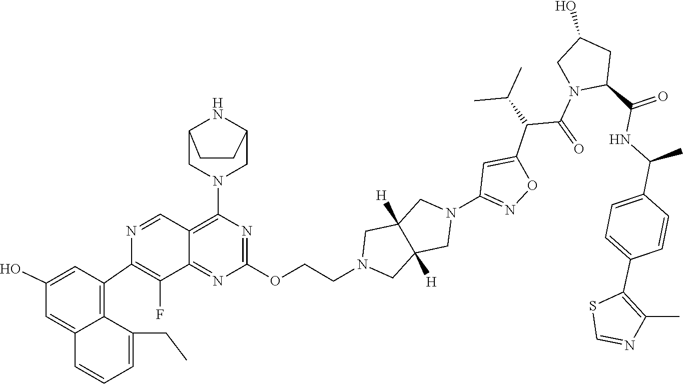

The title compound was made in an analogous manner to Compound 70 starting from tert-butyl(3aS,6aR)-2,3,3a,4,6,6a-hexahydro-1H-pyrrolo[3,4-c]pyrrole-5-carboxylate, and purified by prep-HPLC (column: Unisil 3-100 C18 Ultra 150*50 mm*3 um; mobile phase: [12-42% CH₃CN in water (formic acid)]) followed by prep-HPLC (column: Waters Xbridge 150*25 mm*5 um; mobile phase: [32-62% CH₃CN in water (NH₄HCO₃)]) to afford (2S,4R)-1-[(2R*)-2-{3-[(3aR,6aS)-5-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-octahydropyrrolo[3,4-c]pyrrol-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (11 mg, 0.01 mmol, 12% yield) as a white solid. LC/MS (ESI) m/z: 1064.3 [M+H]⁺. ¹H NMR (400 MHz, DMSO-d₆) δ 9.89 (s, 1H), 9.08 (s, 1H), 9.02-8.88 (m, 1H), 8.17 (d, J=7.6 Hz, 1H), 7.67 (d, J=8.3 Hz, 1H), 7.52-7.25 (m, 6H), 7.12 (d, J=7.0 Hz, 1H), 6.98 (d, J=2.3 Hz, 1H), 5.99 (s, 1H), 5.16-4.95 (m, 1H), 4.92-4.80 (m, 1H), 4.56-4.37 (m, 5H), 4.33-4.20 (m, 1H), 3.69 (d, J=8.9 Hz, 1H), 3.64-3.42 (m, 6H), 3.31 (s, 3H), 3.28-3.24 (m, 1H), 3.06-2.93 (m, 2H), 2.87-2.71 (m, 6H), 2.44 (s, 3H), 2.43-2.35 (m, 2H), 2.31-2.17 (m, 3H), 2.11-1.99 (m, 1H), 1.97-1.76 (m, 1H), 1.68-1.53 (m, 4H), 1.49-1.31 (m, 3H), 0.96 (d, J=6.6 Hz, 2H), 0.87-0.71 (m, 7H).

Exemplary Synthesis of (2S,4R)-1-[(2R)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 81)

Step 1: Preparation of tert-butyl 4-[[iodo(triphenyl)-λ5-phosphanyl]methyl]piperidine-1-carboxylate

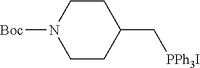

To a solution of tert-butyl 4-(iodomethyl) piperidine-1-carboxylate (25 g, 76.88 mmol, 1 eq) in acetonitrile (125 mL) was added triphenylphosphine (20.77 g, 79.19 mmol, 1.03 eq), and the reaction mixture was stirred at 90° C. for 48 hours. The mixture was concentrated under reduced pressure at 45° C. to give a white solid. Ethyl acetate (120 mL) was then added, and the mixture was allowed to cool slowly to 20° C. The precipitate was collected by filtration, then washed with ethyl acetate (30 mL×3) to afford tert-butyl 4-[[iodo(triphenyl)-λ5-phosphanyl]methyl]piperidine-1-carboxylate (33.06 g, 56.28 mmol, 73% yield) as a white solid. ¹H NMR (400 MHz, DMSO-d₆) δ 7.93-7.82 (m, 9H), 7.81-7.75 (m, 6H), 3.74 (d, J=12.4 Hz, 2H), 3.62 (m, 2H), 2.61 (s, 2H), 1.97-1.85 (m, 1H), 1.38 (s, 2H), 1.35 (s, 9H), 1.27-1.14 (m, 2H).

Step 2: Preparation of tert-butyl 4-[(1-benzhydrylazetidin-3-ylidene)methyl]piperidine-1-carboxylate

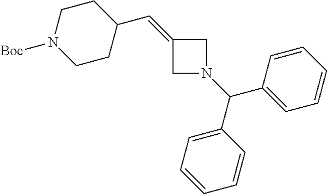 [SMILES: CC(C)(C)OC(=O)N1CCC(C=C2CN(C(c3ccccc3)c3ccccc3)C2)CC1]

To tert-butyl 4-[[iodo(triphenyl)-λ5-phosphanyl]methyl]piperidine-1-carboxylate (28 g, 47.66 mmol, 1 eq) in tetrahydrofuran (250 mL) at 0° C. was added lithium bis(trimethylsilyl)amide (1 M, 71.5 mL, 1.5 eq), and the mixture was stirred at 20° C. for 1 hour. 1-Benzhydrylazetidin-3-one (9.61 g, 40.51 mmol, 0.85 eq) in tetrahydrofuran (50 mL) was then added at 0° C., and the reaction mixture was stirred at 0° C. for 1 hour, then at 20° C. for 10 h. LCMS (EW30959-228-P1A) showed the reaction was completed. The mixture was poured into water (300 mL), stirred for 5 minutes, and the aqueous phase was extracted with ethyl acetate (200 mL×3). The combined organic extract was washed with brine (200 mL×2), dried over anhydrous sodium sulfate, filtered, and concentrated. The resulting residue was purified by flash silica gel chromatography (gradient: 0˜16% ethyl acetate/petroleum ether) to afford tert-butyl 4-[(1-benzhydrylazetidin-3-ylidene)methyl]piperidine-1-carboxylate (5.68 g, 13.57 mmol, 28% yield) as a white solid. LC/MS (ESI) m/z: 419.3 [M+H]⁺. ¹H NMR (400 MHz, DMSO-d₆) δ 7.43 (d, J=7.2 Hz, 4H), 7.27 (t, J=7.5 Hz, 4H), 7.19-7.14 (m, 2H), 5.06 (d, J=8.4 Hz, 1H), 4.55 (s, 1H), 3.86 (d, J=11.6 Hz, 2H), 3.69 (s, 2H), 3.63 (s, 2H), 2.75-2.57 (m, 2H), 2.09-2.00 (m, 1H), 1.54-1.47 (m, 2H), 1.37 (s, 9H), 1.14-1.05 (m, 2H).

Step 3: Preparation of tert-butyl 4-(azetidin-3-ylmethyl) piperidine-1-carboxylate

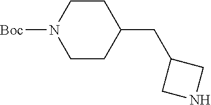 [SMILES: CC(C)(C)OC(=O)N1CCC(CC2CNC2)CC1]

To a solution of tert-butyl 4-[(1-benzhydrylazetidin-3-ylidene)methyl]piperidine-1-carboxylate (7.6 g, 18.16 mmol, 1 eq) in trifluoroethanol (80 mL) was added Pd/C (2 g, 10% purity) under nitrogen, and the resulting suspension was degassed under vacuum and purged with hydrogen several times. The mixture was stirred under hydrogen (15 psi) at 25° C. for 12 hours. The mixture was filtered through a pad of Celite, and the filtrate was concentrated to afford tert-butyl 4-(azetidin-3-ylmethyl) piperidine-1-carboxylate (7 g, crude) as a white oil.

Step 4: Preparation of tert-butyl 4-[[1-[5-(1-methoxycarbonyl-2-methyl-propyl)isoxazol-3-yl]azetidin-3-yl]methyl]piperidine-1-carboxylate

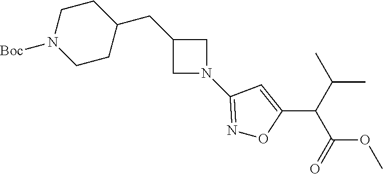 [SMILES: COC(=O)C(c1cc(N2CC(CC3CCN(C(=O)OC(C)(C)C)CC3)C2)no1)C(C)C]

To tert-butyl 4-(azetidin-3-ylmethyl) piperidine-1-carboxylate (4.6 g, 18.08 mmol, 1 eq) in dimethylacetamide (50 mL) at 120° C. was added triethylamine (5.49 g, 54.25 mmol, 7.6 mL, 3 eq) followed by methyl 3-methyl-2-[3-(1,1,2,2,3,3,4,4,4-nonafluorobutylsulfonyloxy) isoxazol-5-yl]butanoate (10.44 g, 21.70 mmol, 1.2 eq) in DMF (100 mL), and the reaction mixture was stirred at 120° C. for 4 hours. The mixture was cooled to 20° C., then poured into water (50 mL) and stirred for 2 minutes. The aqueous phase was extracted with ethyl acetate (20 mL×3), and the combined organic extract was washed with brine (20 mL×2), dried over anhydrous sodium sulfate, filtered, and concentrated. The resulting residue was purified by prep-HPLC (column: Phenomenex luna C18 (250*70 mm, 10 um); mobile phase: [50-80% CH₃CN in water (TFA)]) to afford tert-butyl 4-[[1-[5-(1-methoxy carbonyl-2-methyl-propyl)isoxazol-3-yl]azetidin-3-yl]methyl]piperidine-1-carboxylate (1.7 g, 3.90 mmol, 22% yield) as a white solid. LC/MS (ESI) m/z: 436.3 [M+H]⁺.

Step 5: Preparation of 2-[3-[3-[(1-tert-butoxycarbonyl-4-piperidyl)methyl]azetidin-1-yl]isoxazol-5-yl]-3-methyl-butanoic acid

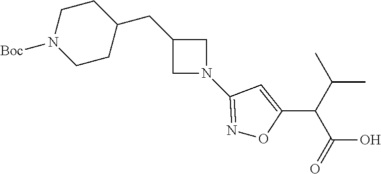 [SMILES: CC(C)C(C(=O)O)c1cc(N2CC(CC3CCN(C(=O)OC(C)(C)C)CC3)C2)no1]

To a solution of tert-butyl 4-[[1-[5-(1-methoxycarbonyl-2-methyl-propyl)isoxazol-3-yl]azetidin-3-yl]methyl]piperidine-1-carboxylate (1.4 g, 3.21 mmol, 1 eq) in tetrahydrofuran (10 mL) and methanol (10 mL) was added lithium hydroxide monohydrate (2 M, 8.0 mL, 5 eq), and the reaction mixture was stirred at 20° C. for 1 hour. The pH of the mixture was adjusted to pH=3 by addition of formic acid. The mixture was poured into water (10 mL), stirred for 2 minutes, and the aqueous phase was extracted with dichloromethane (15 mL×3). The combined organic extract was washed with brine (15 mL×2), dried over anhydrous sodium sulfate, filtered, and concentrated. The resulting residue was purified by prep-HPLC (column: Phenomenex luna C18 (250*70 mm, 10 um); mobile phase: [45-75% CH₃CN in water (formic acid)]) to afford 2-[3-[3-[(1-tert-butoxycarbonyl-4-piperidyl)methyl]azetidin-1-yl]isoxazol-5-yl]-3-methyl-butanoic acid (932 mg, 1.86 mmol, 58% yield) as a yellow solid. LC/MS (ESI) m/z: 422.1 [M+H]⁺. ¹H NMR (400 MHz, DMSO-d₆) δ 5.87 (s, 1H), 4.01-3.84 (m, 4H), 3.52-3.38 (m, 4H), 2.80 (td, J=6.8, 14.0 Hz, 1H), 2.73-2.57 (m, 2H), 2.28-2.16 (m, 1H), 1.59-1.50 (m, 4H), 1.38 (s, 10H), 1.02-0.96 (m, 1H), 0.94 (d, J=6.4 Hz, 3H), 0.82 (d, J=6.8 Hz, 3H).

Step 6: Preparation of tert-butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperidine-1-carboxylate

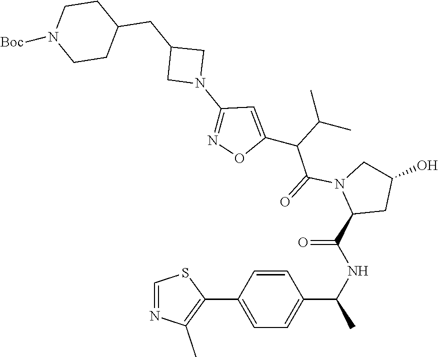 [SMILES: Cc1ncsc1-c1ccc([C@H](C)NC(=O)[C@@H]2C[C@@H](O)CN2C(=O)C(c2cc(N3CC(CC4CCN(C(=O)OC(C)(C)C)CC4)C3)no2)C(C)C)cc1]

To a mixture of 2-[3-[3-[(1-tert-butoxycarbonyl-4-piperidyl)methyl]azetidin-1-yl]isoxazol-5-yl]-3-methyl-butanoic acid (1 g, 2.37 mmol, 1 eq) and (2S,4R)-4-hydroxy-N-[(1S)-1-[4-(4-methylthiazol-5-yl)phenyl]ethyl]pyrrolidine-2-carboxamide (944 mg, 2.56 mmol, 1.08 eq, hydrochloride) in DMF (10 mL) were added N,N-diisopropylethylamine (1.53 g, 11.86 mmol, 2.1 mL, 5 eq) and 2-(7-azobentriazole)-N,N,N′,N′-tetramethyluronium hexafluorophosphate (1.08 g, 2.85 mmol, 1.2 eq), and the reaction mixture was stirred at 0° C. for 0.5 hour. The mixture was poured into ice-water (100 mL) stirred for 2 minutes, and the aqueous phase was extracted with ethyl acetate (50 mL×2). The combined organic extract was washed with brine (50 mL×2), dried over anhydrous sodium sulfate, filtered, and concentrated. The resulting residue was purified by flash silica gel chromatography (gradient: 0˜80% ethyl acetate/petroleum ether) to afford tert-butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperidine-1-carboxylate (1.49 g, 1.80 mmol, 76% yield) as a white solid. LC/MS (ESI) m/z: 735.4 [M+H]⁺.

Step 7: Preparation of tert-butyl 4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperidine-1-carboxylate and tert-butyl 4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperidine-1-carboxylate

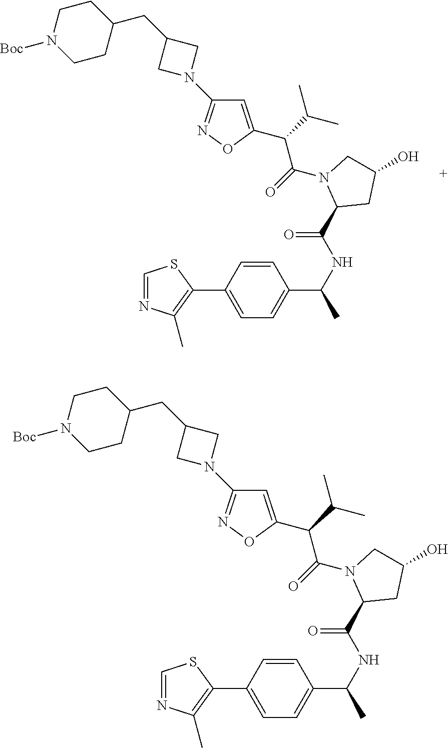 [SMILES: Cc1ncsc1-c1ccc([C@H](C)NC(=O)[C@@H]2C[C@@H](O)CN2C(=O)[C@H](c2cc(N3CC(CC4CCN(C(=O)OC(C)(C)C)CC4)C3)no2)C(C)C)cc1 ; Cc1ncsc1-c1ccc([C@H](C)NC(=O)[C@@H]2C[C@@H](O)CN2C(=O)[C@@H](c2cc(N3CC(CC4CCN(C(=O)OC(C)(C)C)CC4)C3)no2)C(C)C)cc1]

tert-Butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperidine-1-carboxylate (1.64 g, 2.23 mmol, 1 eq) purified by SFC (Column: DAICEL CHIRALPAK IG 250×30 mm, I.D., 10 um; Mobile phase: 60% isopropanol (0.1% NH₄OH) in CO₂; Flow rate: 70 mL/min; Wavelength: 220 nm).

tert-Butyl 4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperidine-1-carboxylate (660 mg, 0.90 mmol, 40% yield) (time: 0.662 min) was obtained as a white solid.

tert-Butyl 4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperidine-1-carboxylate (0.98 g, 1.33 mmol, 60% yield) (time: 1.415 min) was obtained as a white solid.

Step 8: Preparation of (2S,4R)-4-hydroxy-1-[(2R)-3-methyl-2-[3-[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide

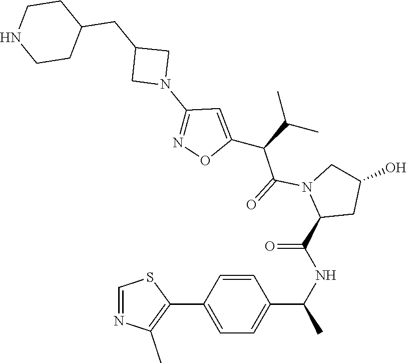 [SMILES: Cc1ncsc1-c1ccc([C@H](C)NC(=O)[C@@H]2C[C@@H](O)CN2C(=O)[C@@H](c2cc(N3CC(CC4CCNCC4)C3)no2)C(C)C)cc1]

To a solution of tert-butyl 4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperidine-1-carboxylate (150 mg, 0.20 mmol, 1 eq) in dichloromethane (1.5 mL) was added trifluoroacetic acid (578 mg, 5.06 mmol, 0.4 mL, 24.82 eq), and the reaction mixture was stirred at 25° C. for 1 hour. The mixture was concentrated in vacuum at 40° C., and the resulting residue was purified by prep-HPLC (column: Phenomenex luna C18 150*40 mm*15 um; mobile phase: [15-45% CH₃CN in water (formic acid)]) to afford (2S,4R)-4-hydroxy-1-[(2R)-3-methyl-2-[3-[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide (80 mg, 0.11 mmol, 52% yield, formic acid salt) as a white solid. LC/MS (ESI) m/z: 635.4 [M+H]⁺.

Step 9: Preparation of tert-butyl 3-[7-(8-ethyl-3-hydroxy-1-naphthyl)-8-fluoro-2-[2-[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate

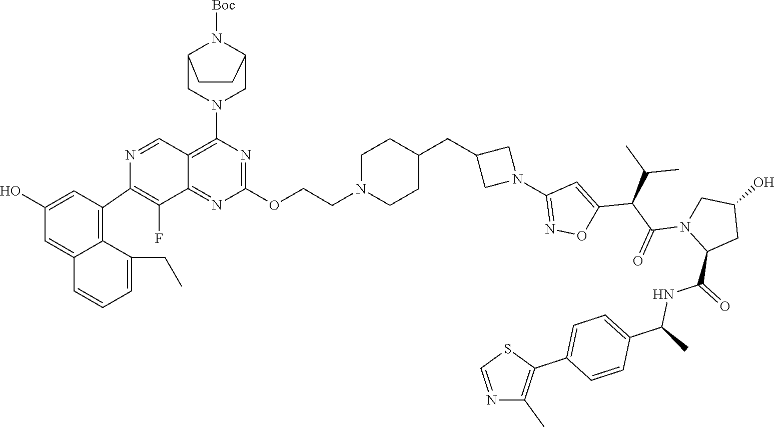 [SMILES: CCc1cccc2cc(O)cc(-c3ncc4c(N5CC6CCC(C5)N6C(=O)OC(C)(C)C)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

To a solution of (2S,4R)-4-hydroxy-1-[(2R)-3-methyl-2-[3-[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide (64 mg, 0.10 mmol, 1.0 eq, formic acid salt) in DMF (2 mL) at 0° C. was added 4-methylmorpholine (26 mg, 0.26 mmol, 0.1 mL, 3 eq), and the resulting mixture was stirred for 10 minutes. tert-Butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (50 mg, 0.1 mmol, 1 eq) was added in it and stirred for 20 min. Sodium triacetoxyborohydride (36 mg, 0.17 mmol, 2 eq) was then added, and the reaction mixture was stirred at 25° C. for 1.5 hours. The mixture was poured into water (10 mL), stirred for 2 minutes, and the aqueous phase was extracted with ethyl acetate (10 mL×2). The combined organic extract was washed with brine (20 mL×2), dried over anhydrous sodium sulfate, filtered, and concentrated. The resulting residue was purified by prep-TLC (SiO₂, dichloromethane:methyl alcohol=10:1) to afford tert-butyl 3-[7-(8-ethyl-3-hydroxy-1-naphthyl)-8-fluoro-2-[2-[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate (52 mg, 0.04 mmol, 51% yield) as a white solid. LC/MS (ESI) m/z: 604.3 [M/2+H]⁺. ¹H NMR (400 MHz, DMSO-d₆) δ 5.87 (s, 1H), 4.01-3.84 (m, 4H), 3.52-3.38 (m, 4H), 2.80 (td, J=6.8, 14.0 Hz, 1H), 2.73-2.57 (m, 2H), 2.28-2.16 (m, 1H), 1.59-1.50 (m, 4H), 1.38 (s, 10H), 1.02-0.96 (m, 1H), 0.94 (d, J=6.4 Hz, 3H), 0.82 (d, J=6.8 Hz, 3H).

Step 10: Preparation of (2S,4R)-1-[(2R)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide

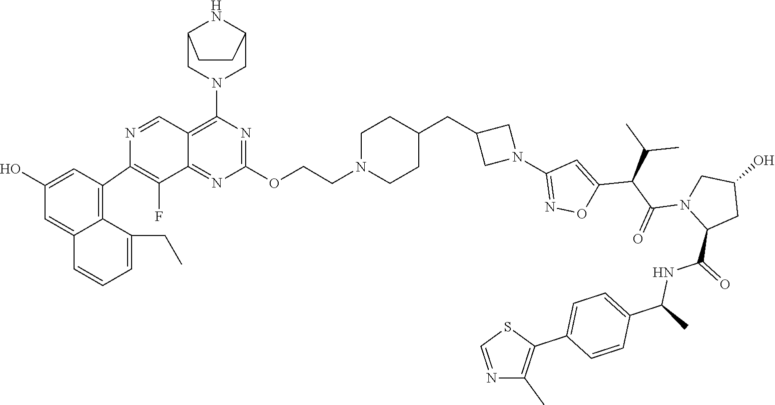 [SMILES: CCc1cccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

A solution of tert-butyl 3-[7-(8-ethyl-3-hydroxy-1-naphthyl)-8-fluoro-2-[2-[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate (150 mg, 0.12 mmol, 1 eq) in trifluoroacetic acid (3.08 g, 27.01 mmol, 2.0 mL, 217.26 eq) and dichloromethane (2 mL) was stirred for 0.5 hours at 25° C. The mixture was concentrated under reduced pressure at 45° C., and the resulting residue was purified by prep-HPLC (column: YMC Triart C18 150*25 mm*5 um; mobile phase: [0-30% CH₃CN in water (formic acid)]) to afford (2S,4R)-1-[(2R)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (97.5 mg, 0.08 mmol, 68% yield, formic acid salt) as a white solid. LC/MS (ESI) m/z: 1106.8 [M+H]⁺. ¹H NMR (400 MHz, DMSO-d₆) δ 9.94 (s, 1H), 9.60-9.30 (m, 1H), 9.17 (s, 1H), 8.99 (s, 1H), 8.39 (d, J=7.6 Hz, 1H), 8.14 (s, 1H), 7.68 (d, J=8.4 Hz, 1H), 7.47-7.41 (m, 2H), 7.39-7.34 (m, 3H), 7.28 (d, J=2.8 Hz, 1H), 7.12 (d, J=6.8 Hz, 1H), 6.97 (d, J=2.8 Hz, 1H), 5.80-5.70 (m, 1H), 5.14-5.07 (m, 1H), 4.91 (quin, J=7.2 Hz, 1H), 4.71-4.55 (m, 4H), 4.38-4.32 (m, 1H), 4.30-4.24 (m, 1H), 4.19-4.10 (m, 2H), 3.99-3.86 (m, 3H), 3.82-3.76 (m, 1H), 3.73-3.67 (m, 1H), 3.60-3.54 (m, 1H), 3.49-3.35 (m, 5H), 2.84-2.74 (m, 1H), 2.53-2.51 (m, 3H), 2.45 (s, 3H), 2.32-2.14 (m, 4H), 2.07-1.95 (m, 1H), 1.95-1.88 (m, 4H), 1.83-1.65 (m, 3H), 1.59-150 (m, 2H), 1.47-1.35 (m, 4H), 1.31-1.19 (m, 2H), 0.97-0.90 (m, 3H), 0.82 (t, J=7.6 Hz, 4H), 0.77 (d, J=6.8 Hz, 2H).

Exemplary Synthesis of (2S,4R)-1-[(2R*)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 82)

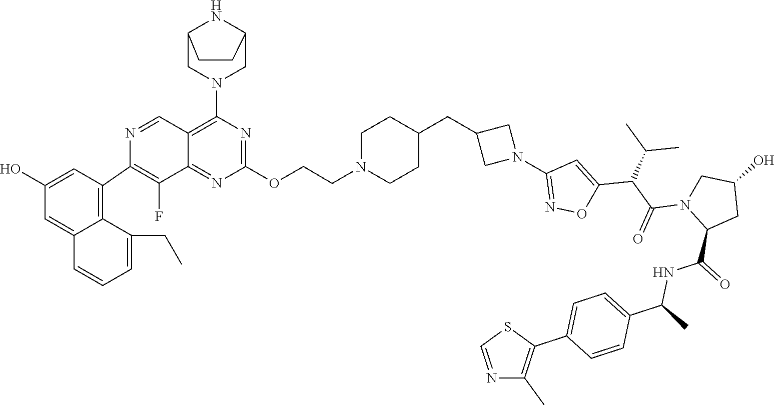

The title compound was prepared in an analogous manner to Compound 81 starting from (2S,4R)-4-hydroxy-1-[(2S)-3-methyl-2-[3-[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate, and purified by prep-HPLC (column: Unisil 3-100 C18 Ultra 150*50 mm*3 um; mobile phase: [12-42% CH₃CN in water (formic acid)]) to afford (2S,4R)-1-[(2R*)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (33.4 mg, 0.03 mmol, 56% yield, formic acid salt) as a white solid. LC/MS (ESI) m/z: 1106.4 [M+H]⁺. ¹H NMR (400 MHz, DMSO-d₆) δ 9.09 (s, 1H), 8.97 (s, 1H), 8.19-8.16 (m, 2H), 7.68-7.64 (m, 1H), 7.49-7.37 (m, 3H), 7.36-7.29 (m, 2H), 7.28 (d, J=2.4 Hz, 1H), 7.12 (d, J=7.6 Hz, 1H), 6.96 (d, J=2.4 Hz, 1H), 5.84-5.82 (m, 1H), 5.02-4.80 (m, 1H), 4.55-4.35 (m, 6H), 4.32-4.18 (m, 1H), 3.94-3.82 (m, 2H), 3.74-3.66 (m, 2H), 3.65-3.59 (m, 4H), 3.53 (d, J=3.6 Hz, 2H), 2.95-2.84 (m, 3H), 2.46-2.43 (m, 3H), 2.29-2.19 (m, 3H), 2.07-1.89 (m, 4H), 1.72-1.64 (m, 4H), 1.56-1.49 (m, 3H), 1.49-1.42 (m, 3H), 1.34 (d, J=7.2 Hz, 2H), 1.27-1.21 (m, 1H), 1.19-1.01 (m, 4H), 0.94 (d, J=6.4 Hz, 3H), 0.84-0.78 (m, 7H).

Exemplary Synthesis of (2S,4R)-1-[(2R*)-2-[3-(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(1-methyl-1H-pyrazol-5-yl)phenyl]ethyl]pyrrolidine-2-carboxamide (Compound 83)

Step 1: Preparation of tert-butyl N-[(1S)-1-[4-(2-methylpyrazol-3-yl)phenyl]ethyl]carbamate

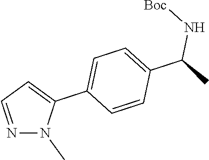 [SMILES: C[C@H](NC(=O)OC(C)(C)C)c1ccc(-c2ccnn2C)cc1]

To a mixture of 5-bromo-1-methyl-pyrazole (800 mg, 4.97 mmol, 1 eq), potassium carbonate (1.37 g, 9.94 mmol, 2 eq) and tert-butyl N-(5-bromothiazol-4-yl) carbamate (2.07 g, 5.96 mmol, 1.2 eq) in water (5 mL) and dioxane (30 mL) was added [1,1′-bis(diphenylphosphino)ferrocene]dichloropalladium (ii) (290 mg, 0.40 mmol, 0.08 eq) in one portion at 20° C. under nitrogen, and the reaction mixture was stirred at 90° C. for 12 hours. The mixture was cooled to 20° C., poured into ice-water (w/w=1/1, 50 mL), and stirred for 10 minutes. The aqueous phase was extracted with ethyl acetate (40 mL×3, and the combined organic extract was washed with brine (20 mL×2), dried over anhydrous sodium sulfate, filtered, and concentrated. The resulting residue was purified by silica gel chromatography (gradient: petroleum ether/ethyl acetate=30/1, 5/1) to afford tert-butyl N-[(1S)-1-[4-(2-methylpyrazol-3-yl)phenyl]ethyl]carbamate (1.6 g) as a yellow solid. ¹H NMR (400 MHz, CDCl₃) δ 7.43 (d, J=2.0 Hz, 1H), 7.33-7.29 (m, 4H), 6.22 (d, J=2.0 Hz, 1H), 4.79 (br s, 2H), 3.82 (s, 3H), 1.36 (br s, 9H).

Step 2: Preparation of (1S)-1-[4-(2-methylpyrazol-3-yl)phenyl]ethanamine

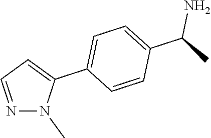

A solution of tert-butyl N-[(1S)-1-[4-(2-methylpyrazol-3-yl)phenyl]ethyl]carbamate (1.6 g, 5.31 mmol, 1 eq) in hydrochloric acid/dioxane (4 M, 20 mL) was stirred at 15° C. for 3 hours. The mixture was concentrated under reduced pressure at 40° C., and the resulting residue was purified by semi-preparative reverse phase HPLC (column: Phenomenex Synergi Max-RP 250*50 mm*10 um; mobile phase: [5-35% CH₃CN in water (0.1% TFA)]) to afford (1S)-1-[4-(2-methylpyrazol-3-yl)phenyl]ethanamine (1.4 g, crude, trifluoroacetate) as a yellow oil. LC/MS (ESI) m/z: 202.2 [M+H]⁺.

Step 3: Preparation of tert-butyl(2S,4R)-4-hydroxy-2-[[(1S)-1-[4-(2-methylpyrazol-3-yl)phenyl]ethyl]carbamoyl]pyrrolidine-1-carboxylate

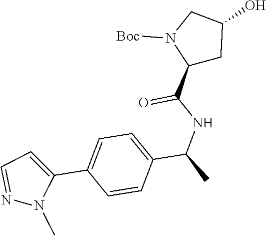 [SMILES: C[C@H](NC(=O)[C@@H]1C[C@@H](O)CN1C(=O)OC(C)(C)C)c1ccc(-c2ccnn2C)cc1]

To a mixture of (1S)-1-[4-(2-methylpyrazol-3-yl)phenyl]ethanamine (3.3 g, 16 mmol, 1 eq, hydrochloride), triethylamine (8.3 g, 82.03 mmol, 11 mL, 5 eq), and (2S,4R)-1-tert-butoxycarbonyl-4-hydroxy-pyrrolidine-2-carboxylic acid (3.8 g, 16 mmol, 1 eq) in DMF (80 mL) was added O-(7-azabenzotriazol-1-yl)-N,N,N,N-tetramethyluronium hexafluorophosphate (8.1 g, 21 mmol, 1 eq) in one portion at 15° C. under nitrogen, and the reaction mixture was stirred at 15° C. for 2 hours. The mixture was poured into ethyl acetate (800 mL), and the organic phase was washed with brine (50 mL), dried over anhydrous sodium sulfate, filtered, and concentrated. The resulting residue was purified by prep-HPLC (column: Phenomenex luna C18 250*50 mm*10 um; mobile phase: [15-45% CH₃CN in water (0.225% formic acid)]) to afford tert-butyl(2S,4R)-4-hydroxy-2-[[(1S)-1-[4-(2-methylpyrazol-3-yl)phenyl]ethyl]carbamoyl]pyrrolidine-1-carboxylate (5.7 g, 13 mmol, 83% yield) as a yellow solid. LC/MS (ESI) m/z: 415.3 [M+H]⁺.

Step 4: Preparation of (2S,4R)-4-hydroxy-N-[(1S)-1-[4-(4-methylthiazol-5-yl)phenyl]ethyl]pyrrolidine-2-carboxamide

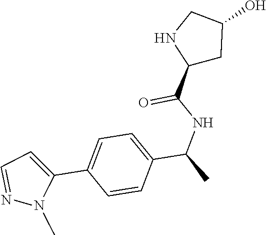 [SMILES: C[C@H](NC(=O)[C@@H]1C[C@@H](O)CN1)c1ccc(-c2ccnn2C)cc1]

A solution of tert-butyl(2S,4R)-4-hydroxy-2-[[(1S)-1-[4-(4-methylthiazol-5-yl)phenyl]ethyl]carbamoyl]pyrrolidine-1-carboxylate (5.7 g, 13.00 mmol, 1 eq) in hydrochloric acid/methanol (4 M, 50 mL, 15 eq) was stirred at 15° C. for 1 hour. The mixture was concentrated in vacuum at 45° C. to afford (2S,4R)-4-hydroxy-N-[(1S)-1-[4-(4-methylthiazol-5-yl)phenyl]ethyl]pyrrolidine-2-carboxamide (4.8 g, crude, HCl salt) as a yellow solid.

Step 5: Preparation of tert-butyl 2-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate

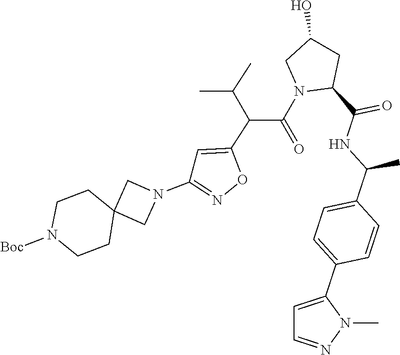 [SMILES: CC(C)C(C(=O)N1C[C@H](O)C[C@H]1C(=O)N[C@@H](C)c1ccc(-c2ccnn2C)cc1)c1cc(N2CC3(CCN(C(=O)OC(C)(C)C)CC3)C2)no1]

To a mixture of (2S,4R)-4-hydroxy-N-[(1S)-1-[4-(2-methylpyrazol-3-yl)phenyl]ethyl]pyrrolidine-2-carboxamide (892 mg, 2.54 mmol, 1 eq, hydrochloric) and 2-[3-(7-tert-butoxycarbonyl-2,7-diazaspiro[3.5]nonan-2-yl)isoxazol-5-yl]-3-methyl-butanoic acid (1 g, 2.54 mmol, 1 eq) in DMF (10 mL) were added [dimethylamino (triazolo[4,5-b]pyridin-3-yloxy)methylidene]-dimethylazanium; hexafluorophosphate (1.45 g, 3.81 mmol, 1.5 eq) and triethylamine (1.29 g, 12.71 mmol, 1.8 mL, 5 eq) in one portion at 20° C. under nitrogen, and the reaction mixture was stirred at 20° C. for 2 hours. The reaction mixture was diluted with water (20 mL) and extracted with ethyl acetate (20 mL×3). The combined organic extract was washed with brine (20 mL), dried with anhydrous sodium sulfate, filtered, and concentrated. The resulting residue was purified by prep-HPLC (column: Phenomenex luna C18 250*50 mm*10 um; mobile phase: [35-65% CH₃CN in water (formic acid)]) to afford tert-butyl 2-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (1.41 g, 2.04 mmol, 80% yield) as a yellow solid. LC/MS (ESI) m/z: 690.3 [M+H]⁺.

Step 6: Preparation of tert-butyl 2-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate and tert-butyl 2-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate

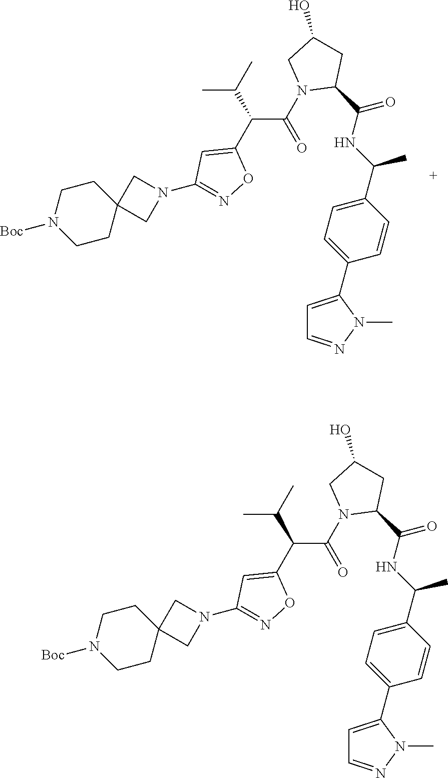 [SMILES: CC(C)[C@H](C(=O)N1C[C@H](O)C[C@H]1C(=O)N[C@@H](C)c1ccc(-c2ccnn2C)cc1)c1cc(N2CC3(CCN(C(=O)OC(C)(C)C)CC3)C2)no1 ; CC(C)[C@@H](C(=O)N1C[C@H](O)C[C@H]1C(=O)N[C@@H](C)c1ccc(-c2ccnn2C)cc1)c1cc(N2CC3(CCN(C(=O)OC(C)(C)C)CC3)C2)no1]

The compound tert-butyl 2-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (1.4 g, 2.03 mmol, 1 eq) was separated by chiral SFC (column: DAICEL CHIRALPAK IE (250 mm*30 mm, 10 um); mobile phase: [65% EtOH (0.1% NH₄OH)]).

tert-Butyl 2-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (408 mg, 0.59 mmol, 29% yield) was obtained as a yellow solid. (Rt=1.020)

tert-Butyl 2-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (660 mg, 0.96 mmol, 47% yield) was obtained as a yellow solid. (Rt=1.596).

Step 7: Preparation of (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(2-methylpyrazol-3-yl)phenyl]ethyl]pyrrolidine-2-carboxamide

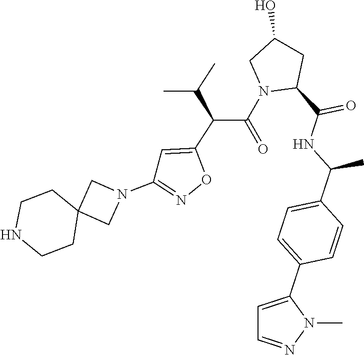 [SMILES: CC(C)[C@@H](C(=O)N1C[C@H](O)C[C@H]1C(=O)N[C@@H](C)c1ccc(-c2ccnn2C)cc1)c1cc(N2CC3(CCNCC3)C2)no1]

A solution of tert-butyl 2-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-2,7-diazaspiro[3.5]nonane-7-carboxylate (70 mg, 0.10 mmol, 1 eq) in trifluoroacetic acid (2.12 g, 18.61 mmol, 1.4 mL, 183.36 eq) and dichloromethane (1 mL) was stirred for 0.5 hours at 20° C. The mixture was concentrated to afford (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(2-methylpyrazol-3-yl)phenyl]ethyl]pyrrolidine-2-carboxamide (70 mg, 0.10 mmol, 98% yield, TFA salt) as a yellow oil. LC/MS (ESI) m/z: 590.3 [M+H]⁺.

Step 8: Preparation of (2S,4R)-1-[(2R*)-2-[3-(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(1-methyl-1H-pyrazol-5-yl)phenyl]ethyl]pyrrolidine-2-carboxamide

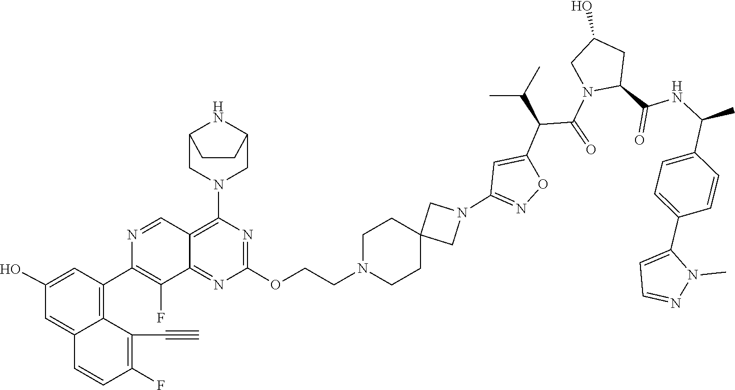 [SMILES: C#Cc1c(F)ccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8ccnn8C)cc7)C(C)C)on5)C6)nc4c3F)c12]

The title compound was made in an analogous manner to Compound 33 starting from tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate and (2S,4R)-1-[(2R)-2-[3-(2,7-diazaspiro[3.5]nonan-2-yl)isoxazol-5-yl]-3-methyl-butanoyl]-4-hydroxy-N-[(1S)-1-[4-(2-methylpyrazol-3-yl)phenyl]ethyl]pyrrolidine-2-carboxamide, and purified by prep-HPLC {column: Unisil 3-100 C18 Ultra 150*50 mm*3 um; mobile phase: [11-41% CH₃CN in water (formic acid)]} to afford (2S,4R)-1-[(2R*)-2-[3-(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(1-methyl-1H-pyrazol-5-yl)phenyl]ethyl]pyrrolidine-2-carboxamide (49.3 mg, 0.04 mmol, 40% yield, formic acid salt) as a yellow solid. LC/MS (ESI) m/z: 1075.6 [M+H]⁺. ¹H NMR (400 MHz, DMSO-d₆) δ 10.13 (s, 1H), 9.06 (s, 1H), 8.41 (d, J=7.6 Hz, 1H), 8.16 (s, 1H), 8.00-7.95 (m, 1H), 7.50-7.43 (m, 4H), 7.40-7.33 (m, 3H), 7.17 (d, J=2.4 Hz, 1H), 6.36 (d, J=2.0 Hz, 1H), 5.83 (s, 1H), 4.97-4.88 (m, 1H), 4.57-4.50 (m, 1H), 4.47-4.44 (m, 1H), 4.42-4.32 (m, 2H), 4.30-4.25 (m, 1H), 3.94 (s, 1H), 3.85 (s, 3H), 3.80-3.62 (m, 5H), 3.60-3.50 (m, 6H), 3.45-3.35 (m, 3H), 2.72-2.65 (m, 2H), 2.45-2.37 (m, 4H), 2.25-2.10 (m, 2H), 2.05-1.95 (m, 1H), 1.80-1.65 (m, 8H), 1.47-1.35 (m, 3H), 1.00-0.92 (m, 3H), 0.85-0.75 (m, 3H).

Exemplary Synthesis of (2S,4R)-1-[(2R*)-2-[3-(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(1-methyl-1H-pyrazol-5-yl)phenyl]ethyl]pyrrolidine-2-carboxamide (Compound 84)

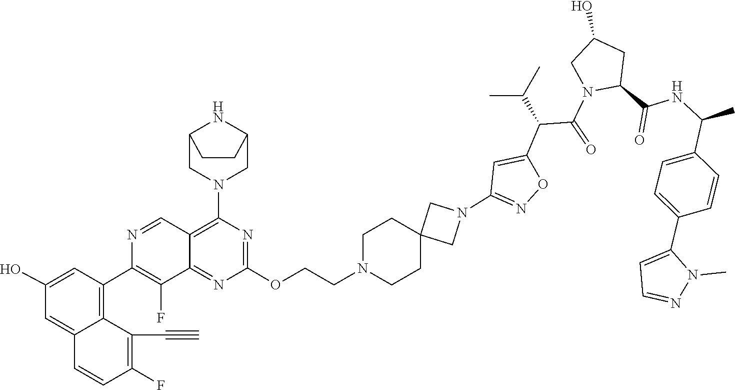 [SMILES: C#Cc1c(F)ccc2cc(O)cc(-c3ncc4c(N5CC6CCC(C5)N6)nc(OCCN5CCC6(CC5)CN(c5cc([C@@H](C(=O)N7C[C@H](O)C[C@H]7C(=O)N[C@@H](C)c7ccc(-c8ccnn8C)cc7)C(C)C)on5)C6)nc4c3F)c12]

The title compound was made in an analogous manner to Compound 33 starting from tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate and (2S,4R)-1-[(2S)-2-[3-(2,7-diazaspiro[3.5]nonan-2-yl)isoxazol-5-yl]-3-methyl-butanoyl]-4-hydroxy-N-[(1S)-1-[4-(2-methylpyrazol-3-yl)phenyl]ethyl]pyrrolidine-2-carboxamide, and purified by prep-HPLC {column: Unisil 3-100 C18 Ultra 150*50 mm*3 um; mobile phase: [9-39% CH₃CN in water (formic acid)]} to afford (2S,4R)-1-[(2R*)-2-[3-(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(1-methyl-1H-pyrazol-5-yl)phenyl]ethyl]pyrrolidine-2-carboxamide (43.5 mg, 0.04 mmol, 53% yield, formic acid salt) as a yellow solid. LC/MS (ESI) m/z: 1075.6 [M+H]⁺. ¹H NMR (400 MHz, DMSO-d₆) δ 10.13 (s, 1H), 9.06 (s, 1H), 8.23 (d, J=8.4 Hz, 1H), 8.16 (s, 1H), 8.00-7.95 (m, 1H), 7.53-7.48 (m, 1H), 7.47-7.42 (m, 4H), 7.41-7.38 (m, 1H), 7.35-7.32 (m, 1H), 7.17 (d, J=2.4 Hz, 1H), 6.40-6.34 (m, 1H), 5.86 (s, 1H), 4.94-4.84 (m, 1H), 4.58-4.50 (m, 1H), 4.48-4.34 (m, 4H), 4.30-4.23 (m, 1H), 3.94 (s, 1H), 3.86-3.82 (m, 3H), 3.78-3.72 (m, 2H), 3.71-3.62 (m, 3H), 3.58-3.45 (m, 7H), 2.71-2.62 (m, 2H), 2.44-2.35 (m, 4H), 2.26-2.16 (m, 2H), 2.17-1.95 (m, 1H), 1.80-1.65 (m, 9H), 1.46-1.32 (m, 3H), 0.95 (d, J=6.4 Hz, 2H), 0.83-0.73 (m, 4H).

Exemplary Synthesis of (2S,4R)-1-[(2R*)-2-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(1-methyl-1H-pyrazol-5-yl)phenyl]ethyl]pyrrolidine-2-carboxamide (Compound 85)

Step 1: Preparation of tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate

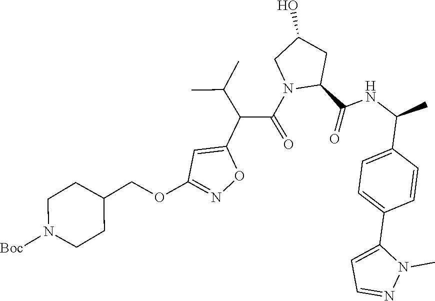 [SMILES: CC(C)C(C(=O)N1C[C@H](O)C[C@H]1C(=O)N[C@@H](C)c1ccc(-c2ccnn2C)cc1)c1cc(OCC2CCN(C(=O)OC(C)(C)C)CC2)no1]

To a mixture of (2S,4R)-4-hydroxy-N-[(1S)-1-[4-(2-methylpyrazol-3-yl)phenyl]ethyl]pyrrolidine-2-carboxamide (3.08 g, 8.79 mmol, 1.2 eq, hydrochloric) and 2-[3-[(1-tert-butoxycarbonyl-4-piperidyl)methoxy]isoxazol-5-yl]-3-methyl-butanoic acid (2.8 g, 7.32 mmol, 1 eq) in DMF (20 mL) were added O-(7-Azabenzotriazol-1-yl)-N,N,N′,N′-tetramethyluronium Hexafluorophosphate (4.18 g, 10.98 mmol, 1.5 eq) and triethylamine (3.70 g, 36.61 mmol, 5.1 mL, 5 eq) in one portion at 20° C. under nitrogen, and the reaction mixture was stirred at 20° C. for 2 h. The reaction mixture was diluted with water (20 mL) and extracted with ethyl acetate (20 mL×3). The combined organic extract was washed with brine (20 mL), dried over anhydrous sodium sulfate, filtered, and concentrated. The resulting residue was purified by prep-HPLC (column: Phenomenex luna C18 (250*70 mm, 10 um); mobile phase: [40-70% CH₃CN in water (formic acid)]) to afford tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (3.3 g, 4.86 mmol, 66% yield) as a yellow solid. LC/MS (ESI) m/z: 696.3 [M+18]⁺.

Step 2: Preparation of tert-butyl 4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate and tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate

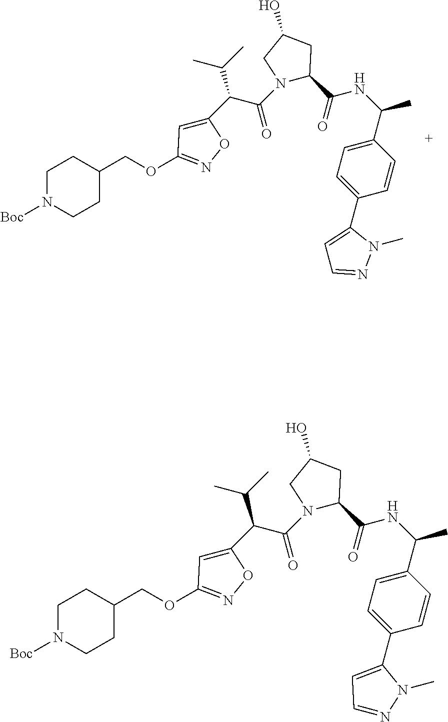 [SMILES: CC(C)[C@H](C(=O)N1C[C@H](O)C[C@H]1C(=O)N[C@@H](C)c1ccc(-c2ccnn2C)cc1)c1cc(OCC2CCN(C(=O)OC(C)(C)C)CC2)no1 ; CC(C)[C@@H](C(=O)N1C[C@H](O)C[C@H]1C(=O)N[C@@H](C)c1ccc(-c2ccnn2C)cc1)c1cc(OCC2CCN(C(=O)OC(C)(C)C)CC2)no1]

The tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (2.7 g, 3.98 mmol, 1 eq) was separated by chiral SFC (column: DAICEL CHIRALPAK IE (250 mm*30 mm, 10 um); mobile phase: [50% EtOH (0.1% NH₄OH)]).

tert-Butyl 4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (620 mg, 0.91 mmol, 23% yield) was obtained as a yellow solid. (Rt=1.884)

tert-Butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate (1.31 g, 1.93 mmol, 48% yield) was obtained as a yellow solid. (Rt=2.228)

Step 3: Preparation of (2S,4R)-4-hydroxy-1-[(2R)-3-methyl-2-[3-(4-piperidylmethoxy)isoxazol-5-yl]butanoyl]-N-[(1S)-1-[4-(2-methylpyrazol-3-yl)phenyl]ethyl]pyrrolidine-2-carboxamide

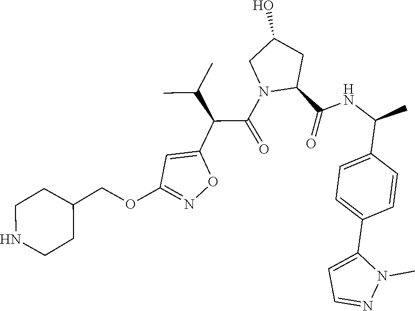 [SMILES: CC(C)[C@@H](C(=O)N1C[C@H](O)C[C@H]1C(=O)N[C@@H](C)c1ccc(-c2ccnn2C)cc1)c1cc(OCC2CCNCC2)no1]

A solution of tert-butyl 4-[[5-[(1R)-1-[(2S,4R)-4-hydroxy-2-[[(1S)-1-[4-(2-yl)phenyl]ethyl]carbamoyl]pyrrolidine-1-carbonyl]-2-methyl-methylpyrazol-3-propyl]isoxazol-3-yl]oxymethyl]piperidine-1-carboxylate (250 mg, 0.37 mmol, 1 eq) in trifluoroacetic acid (4.62 g, 40.52 mmol, 3 mL, 110.02 eq) and dichloromethane (3 mL) was stirred for 0.5 h at 20° C. The mixture was concentrated to afford (2S,4R)-4-hydroxy-1-[(2R)-3-methyl-2-[3-(4-piperidylmethoxy)isoxazol-5-yl]butanoyl]-N-[(1S)-1-[4-(2-methylpyrazol-3-yl)phenyl]ethyl]pyrrolidine-2-carboxamide (250 mg, 0.36 mmol, 98% yield, TFA salt) as a yellow oil. LC/MS (ESI) m/z: 579.5 [M+H]⁺.

Step 4: Preparation of (2S,4R)-1-[(2R)-2-[3-[[1-[2-[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide

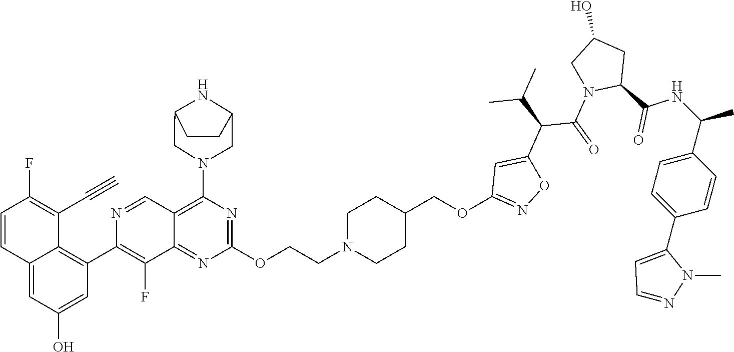 [SMILES: C#Cc1c(F)ccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8ccnn8C)cc7)C(C)C)on6)CC5)nc4c3F)c12]

The title compound was prepared in an analogous manner to Compound 39 starting from (2S,4R)-4-hydroxy-1-[(2R)-3-methyl-2-[3-(4-piperidylmethoxy)isoxazol-5-yl]butanoyl]-N-[(1S)-1-[4-(2-methylpyrazol-3-yl)phenyl]ethyl]pyrrolidine-2-carboxamide and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate, and purified by prep-HPLC {column: Unisil 3-100 C18 Ultra 150*50 mm*3 um; mobile phase: [10-40% CH₃CN in water (formic acid)]} to afford (2S,4R)-1-[(2R*)-2-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(1-methyl-1H-pyrazol-5-yl)phenyl]ethyl]pyrrolidine-2-carboxamide (46.7 mg, 0.04 mmol, 40% yield, formic acid salt) as a yellow solid. LC/MS (ESI) m/z: 1064.5 [M+H]⁺. ¹H NMR (400 MHz, CDCl₃) δ 10.18 (s, 1H), 9.07 (s, 1H), 8.42 (d, J=7.6 Hz, 1H), 8.14 (s, 1H), 8.02-7.95 (m, 1H), 7.51-7.44 (m, 4H), 7.41-7.36 (m, 3H), 7.17 (d, J=2.4 Hz, 1H), 6.36 (d, J=2.0 Hz, 1H), 6.06 (s, 1H), 4.97-4.88 (m, 1H), 4.62-4.55 (m, 1H), 4.50-4.45 (m, 2H), 4.43-4.33 (m, 2H), 4.30-4.23 (m, 1H), 4.02-3.96 (m, 2H), 3.94 (s, 1H), 3.91-3.87 (m, 2H), 3.84 (s, 3H), 3.76-3.67 (m, 3H), 3.66-3.61 (m, 1H), 3.47-3.40 (m, 2H), 3.04-2.95 (m, 2H), 2.78-2.71 (m, 2H), 2.28-2.15 (m, 2H), 2.12-1.98 (m, 3H), 1.86-1.79 (m, 4H), 1.78-1.66 (m, 4H), 1.47-1.36 (m, 3H), 1.34-1.22 (m, 2H), 0.98-0.92 (m, 3H), 0.85-0.75 (m, 3H).

Exemplary Synthesis of (2S,4R)-1-[(2R*)-2-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(1-methyl-1H-pyrazol-5-yl)phenyl]ethyl]pyrrolidine-2-carboxamide (Compound 86)

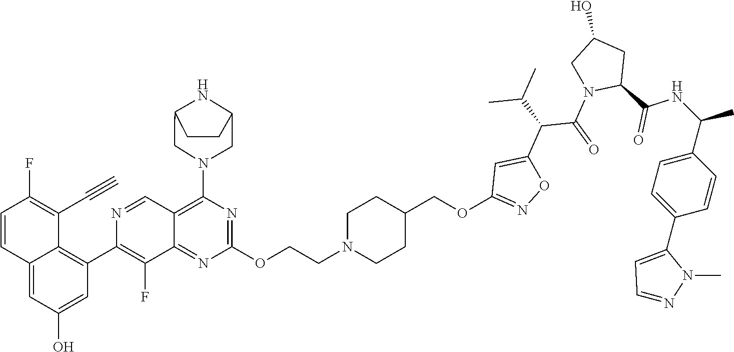 [SMILES: C#Cc1c(F)ccc2cc(O)cc(-c3ncc4c(N5CC6CCC(C5)N6)nc(OCCN5CCC(COc6cc([C@@H](C(=O)N7C[C@H](O)C[C@H]7C(=O)N[C@@H](C)c7ccc(-c8ccnn8C)cc7)C(C)C)on6)CC5)nc4c3F)c12]

The title compound was prepared in an analogous manner to Compound 39 starting from (2S,4R)-4-hydroxy-1-[(2S)-3-methyl-2-[3-(4-piperidylmethoxy)isoxazol-5-yl]butanoyl]-N-[(1S)-1-[4-(2-methylpyrazol-3-yl)phenyl]ethyl]pyrrolidine-2-carboxamide and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate, and purified by prep-HPLC {column: Unisil 3-100 C18 Ultra 150*50 mm*3 um; mobile phase: [9-39% CH₃CN in water (formic acid)]} to afford (2S,4R)-1-[(2R*)-2-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(1-methyl-1H-pyrazol-5-yl)phenyl]ethyl]pyrrolidine-2-carboxamide (57.5 mg, 0.05 mmol, 41.78% yield, formic acid salt) as a yellow solid. LC/MS (ESI) m/z: 1064.5 [M+H]⁺. ¹H NMR (400 MHz, CDCl₃) δ 10.18 (s, 1H), 9.07 (s, 1H), 8.32 (d, J=8.0 Hz, 1H), 8.15 (s, 1H), 8.01-7.95 (m, 1H), 7.54-7.48 (m, 1H), 7.47-7.42 (m, 4H), 7.41-7.39 (m, 1H), 7.36-7.31 (m, 2H), 6.39-6.34 (m, 1H), 6.13-6.06 (m, 1H), 4.97-4.85 (m, 1H), 4.64-4.55 (m, 1H), 4.52-4.40 (m, 4H), 4.30-4.23 (m, 1H), 4.02-3.90 (m, 5H), 3.85-3.82 (m, 3H), 3.78-3.70 (m, 3H), 3.57-3.53 (m, 1H), 3.50-3.42 (m, 3H), 3.03-2.95 (m, 2H), 2.78-2.71 (m, 2H), 2.30-2.17 (m, 2H), 2.13-2.02 (m, 3H), 1.86-1.82 (m, 3H), 1.77-1.65 (m, 4H), 1.47-1.33 (m, 3H), 1.30-1.20 (m, 2H), 0.96 (d, J=6.8 Hz, 2H), 0.85-0.72 (m, 4H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 87)

Step 1: Preparation of [(3R,8S)-3-[[tert-butyl(diphenyl)silyl]oxymethyl]-1,2,3,5,6,7-hexahydropyrrolizin-8-yl]methanol and [(3S,8R)-3-[[tert-butyl(diphenyl)silyl]oxymethyl]-1,2,3,5,6,7-hexahydropyrrolizin-8-yl]methanol

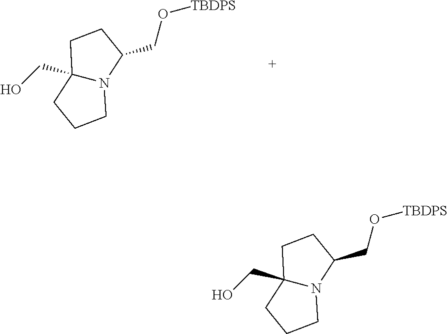

[cis-3-[[tert-butyl(diphenyl)silyl]oxymethyl]-1,2,3,5,6,7-hexahydropyrrolizin-8-yl]methanol (4.8 g, 11.72 mmol) was purified by SFC (column: DAICEL CHIRALCEL OX (250 mm*50 mm, 10 um); mobile phase: [0.1% NH₃H₂O IPA]; B %: 45%-45%) to afford [(3R,8S)-3-[[tert-butyl(diphenyl)silyl]oxymethyl]-1,2,3,5,6,7-hexahydropyrrolizin-8-yl]methanol (1.5 g, 31%) as light yellow oil and [(3S,8R)-3-[[tert-butyl(diphenyl)silyl]oxymethyl]-1,2,3,5,6,7-hexahydropyrrolizin-8-yl]methanol (2.6 g, 54%) as light yellow oil. MS (ESI) m/z: 410.3 [M+H]⁺.

Step 2: Preparation of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

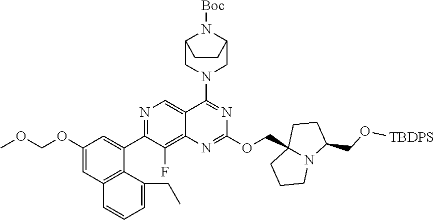

To a solution of tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (875 mg, 1.34 mmol, 1.1 eq) and [(3S,8R)-3-[[tert-butyl(diphenyl)silyl]oxymethyl]-1,2,3,5,6,7-hexahydropyrrolizin-8-yl]methanol (500 mg, 1.22 mmol, 1.0 eq) in toluene (16 mL) were added t-BuONa (352 mg, 3.66 mmol, 3.0 eq) and 4 A MS (100 mg) at 0° C. The mixture was stirred at 0° C. for 1 hour under N₂. The reaction mixture was quenched with sat. aq. NH₄Cl (15 mL) and extracted with ethyl acetate (20 mL×3). The combined organic layer was washed with brine (50 mL), dried over Na₂SO₄, filtered and concentrated under reduced pressure. The residue was purified by silica gel column (0˜23% ethyl acetate in petroleum ether; Gradient time: 15 min; Hold time: 15 min; Flow rate: 40 mL/min) to afford tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (586 mg, 26%, 54% purity) as a yellow oil. MS (ESI) m/z: 981.3 [M+H]⁺.

Step 3: Preparation of [(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 4-[2-(2,6-dioxo-3-piperidyl)-1-oxo-isoindolin-5-yl]piperazine-1-carboxylate

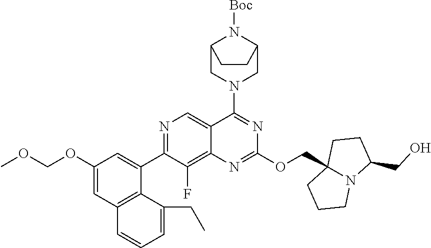 [SMILES: CCc1cccc2cc(OCOC)cc(-c3ncc4c(N5CC6CCC(C5)N6C(=O)OC(C)(C)C)nc(OC[C@]56CCCN5[C@H](CO)CC6)nc4c3F)c12]

To a solution of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (586 mg, 0.322 mmol, 54% purity, 1.0 eq) in THF (5 mL) was added TBAF (1 M, 387 μL, 1.2 eq). The mixture was stirred at 25° C. for 3 hours. The reaction mixture was diluted with ethyl acetate (30 mL), and then washed with water (20 mL×5). The combined organic phase was dried over anhydrous sodium sulfate, filtered and concentrated in vacuum to afford tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (550 mg, 94%) as yellow oil. MS (ESI) m/z: 743.2 [M+H]⁺.

Step 4: Preparation of tert-butyl 3-[7-[8-ethyl-3-(methoxymethoxy)-1-naphthyl]-8-fluoro-2-[[(3S,8R)-3-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

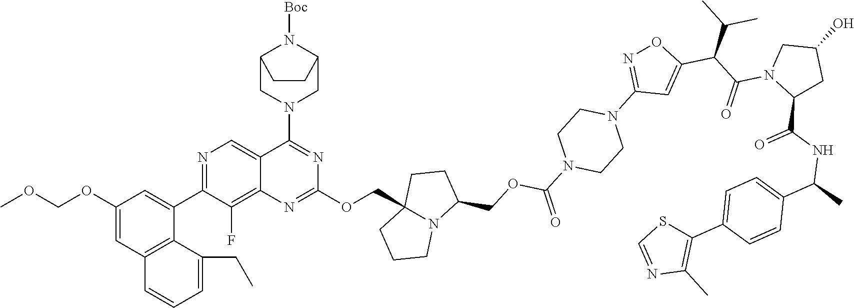 [SMILES: CCc1cccc2cc(OCOC)cc(-c3ncc4c(N5CC6CCC(C5)N6C(=O)OC(C)(C)C)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (400 mg, 0.538 mmol, 1.0 eq) in THF (5 mL) were added TEA (4.31 mmol, 600 μL, 8.0 eq), DMAP (6.58 mg, 0.054 mmol, 0.1 eq) and (4-nitrophenyl) carbonochloridate (206 mg, 1.02 mmol, 1.9 eq). The reaction mixture was stirred at 40° C. for 15 hours. Then (2S,4R)-4-hydroxy-1-[(2S)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide (336 mg, 0.592 mmol, 1.1 eq) was added to the mixture, and the reaction mixture was stirred at 40° C. for 1 hour. The mixture was concentrated, purified by silica gel column (0˜5% methanol in dichloromethane; Gradient time: 10 min; Hold time: 12 min; Flow rate: 30 mL/min) to afford tert-butyl 3-[7-[8-ethyl-3-(methoxymethoxy)-1-naphthyl]-8-fluoro-2-[[(3S,8R)-3-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (375 mg, 52%) as a yellow solid. MS (ESI) m/z: 1335.7 [M+H]⁺.

Step 5: Preparation of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

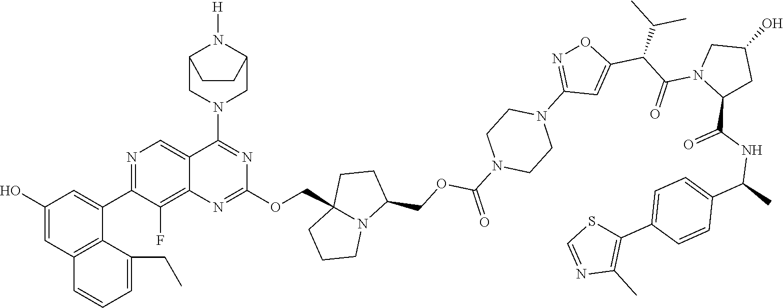 [SMILES: CCc1cccc2cc(O)cc(-c3ncc4c(N5CC6CCC(C5)N6)nc(OC[C@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-[8-ethyl-3-(methoxymethoxy)-1-naphthyl]-8-fluoro-2-[[(3S,8R)-3-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (375 mg, 0.28 mmol, 1.0 eq) in CH₂Cl₂ (3 mL) was added HCl/dioxane (4 M, 2.8 mL). The reaction mixture was stirred at 25° C. for 1 hour. The mixture was concentrated at 25° C. and diluted with CH₂Cl₂ (20 mL) and added TEA (100 uL). Then the mixture was concentrated, the residue was purified by prep-HPLC (column: Phenomenex C18 75*30 mm*3 um; mobile phase: [water (FA)-ACN]; B %: 12%-42%, 25 min). Pure fraction was lyophilized to afford [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (242.8 mg, 69%, FA) as a white solid. MS (ESI) m/z: 1191.7 [M+1]⁺; 1H NMR (400 MHz, CD₃OD) δ 9.10 (d, J=3.6 Hz, 1H), 8.89-8.82 (m, 1H), 8.37 (s, 2H), 7.62 (d, J=8.0 Hz, 1H), 7.46-7.32 (m, 5H), 7.29-7.25 (m, 1H), 7.15 (d, J=6.8 Hz, 1H), 7.02 (t, J=2.4 Hz, 1H), 6.11 (d, J=6.0 Hz, 1H), 5.00-4.94 (m, 1H), 4.64-4.45 (m, 4H), 4.44-4.30 (m, 2H), 4.19-4.02 (m, 3H), 3.94-3.79 (m, 2H), 3.77-3.63 (m, 3H), 3.61-3.35 (m, 7H), 3.23-3.08 (m, 4H), 2.48-2.43 (m, 3H), 2.41-2.14 (m, 7H), 2.10-1.90 (m, 11H), 1.47 (dd, J=3.6, 7.2 Hz, 3H), 1.06 (d, J=6.4 Hz, 3H), 0.92-0.85 (m, 6H).

Exemplary Synthesis of (2S,4R)-1-[(2R)-2-[3-(7-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 88)

Step 1: Preparation of methyl 2-[3-(2,7-diazaspiro[3.5]nonan-2-yl)isoxazol-5-yl]-3-methyl-butanoate

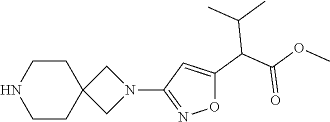 [SMILES: COC(=O)C(c1cc(N2CC3(CCNCC3)C2)no1)C(C)C]

To a mixture of tert-butyl 2-[5-(1-methoxycarbonyl-2-methyl-propyl)isoxazol-3-yl]-2,7-diazaspiro[3.5]nonane-7-carboxylate (495 mg, 1.21 mmol, 1.0 eq) in dichloromethane (6 mL) was added TFA (2 mL) in one portion at 20° C. The mixture was stirred at 20° C. for 1.5 hours. The reaction mixture was concentrated in vacuum, adjusted the pH to 10 by addition of saturated aqueous Na₂CO₃. The resulting mixture was extracted with dichlormethane/methanol (110 mL, 10:1, V/V). The combined organic phase was washed with brine (30 mL), dried over anhydrous Na₂SO₄, filtered and concentrated in vacuum to afford methyl 2-[3-(2,7-diazaspiro[3.5]nonan-2-yl)isoxazol-5-yl]-3-methyl-butanoate (373 mg, 89%) as a yellow solid. MS (ESI) m/z: 308.0 [M+H]⁺.

Step 2: Preparation of (3S,8S)-8-[[tert-butyl(diphenyl)silyl]oxymethyl]-1,2,3,5,6,7-hexahydropyrrolizine-3-carbaldehyde

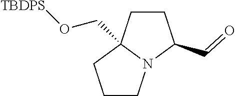

To a solution of DMSO (0.389 mL, 4.98 mmol, 3.0 eq) in CH₂Cl₂ (10 mL) was added was dropwise added a solution of oxalyl dichloride (0.320 mL, 3.65 mmol, 2.2 eq) in CH₂Cl₂ (1 mL) at −78° C. under N₂. After stirred for 10 min, [(3S,8S)-8-[[tert-butyl(diphenyl)silyl]oxymethyl]-1,2,3,5,6,7-hexahydropyrrolizin-3-yl]methanol (680 mg, 1.66 mmol, 1.0 eq) in CH₂Cl₂ (5 mL) was added dropwise and stirred for 20 minutes, then TEA (2.54 mL, 18.26 mmol, 11.0 eq) was added and stirred at −78° C. for 15 minutes. The reaction mixtures was quenched by sat. NaHCO₃ (15 mL), the aqueous phase was extracted with dichloromethane (30 mL×4). The combined organic phase was washed with brine (20 mL), dried with anhydrous Na₂SO₄, filtered, and concentrated in vacuum (at 15° C.) to give product in about 10 mL CH₂Cl₂, which was directly used in the next step. MS (ESI) m/z: 426.2 [M+18]⁺.

Step 3: Preparation of methyl 2-[3-[7-[[(3S,8S)-8-[[tert-butyl(diphenyl)silyl]oxymethyl]-1,2,3,5,6,7-hexahydropyrrolizin-3-yl]methyl]-2,7-diazaspiro[3.5]nonan-2-yl]isoxazol-5-yl]-3-methyl-butanoate

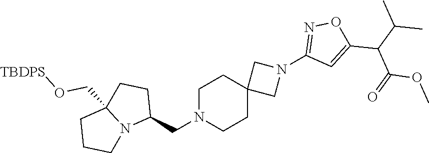

To a solution of (3S,8S)-8-[[tert-butyl(diphenyl)silyl]oxymethyl]-1,2,3,5,6,7-hexahydropyrrolizine-3-carbaldehyde (742 mg, 1.82 mmol, 1.5 eq) and methyl 2-[3-(2,7-diazaspiro[3.5]nonan-2-yl)isoxazol-5-yl]-3-methyl-butanoate (373 mg, 1.21 mmol, 1.0 eq) in dichloromethane (12 mL) was added NaBH(OAc)₃ (566 mg, 2.67 mmol, 2.2 eq). The mixture was stirred at 20° C. for 15 hours. The mixture was quenched with sat. NaHCO₃ (20 mL), and then extracted with dichloromethane (40 mL×3). The combined organic phase was washed with brine (20 mL), dried over anhydrous sodium sulfate, filtered, and concentrated in vacuum, the residue was purified by silica gel column (0˜2 then up to 10% methanol in dichloromethane; Gradient time: 10 min; Hold time: 35 min; Flow rate: 30 mL/min) to afford methyl 2-[3-[7-[[(3S,8S)-8-[[tert-butyl(diphenyl)silyl]oxymethyl]-1,2,3,5,6,7-hexahydropyrrolizin-3-yl]methyl]-2,7-diazaspiro[3.5]nonan-2-yl]isoxazol-5-yl]-3-methyl-butanoate (617 mg, 63%) as a yellow solid. MS (ESI) m/z: 699.3 [M+H]⁺.

Step 4: Preparation of methyl 2-[3-[7-[[(3S,8S)-8-(hydroxymethyl)-1,2,3,5,6,7-hexahydropyrrolizin-3-yl]methyl]-2,7-diazaspiro[3.5]nonan-2-yl]isoxazol-5-yl]-3-methyl-butanoate

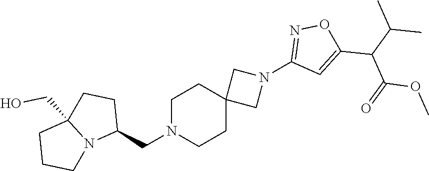 [SMILES: COC(=O)C(c1cc(N2CC3(CCN(C[C@@H]4CC[C@]5(CO)CCCN45)CC3)C2)no1)C(C)C]

To a solution of methyl 2-[3-[7-[[(3S,8S)-8-[[tert-butyl(diphenyl)silyl]oxymethyl]-1,2,3,5,6,7-hexahydropyrrolizin-3-yl]methyl]-2,7-diazaspiro[3.5]nonan-2-yl]isoxazol-5-yl]-3-methyl-butanoate (517 mg, 0.740 mmol, 1.0 eq) in dichloromethane (6 mL) was added N,N-diethylethanamine; trihydrofluoride (11.09 mmol, 1.81 mL, 15.0 eq). The mixture was stirred at 20° C. for 2 hours. The mixture was concentrated under reduced pressure, the residue was diluted with water (20 mL), extracted with MTBE (30 mL×3). The aqueous phase was basified with sat. NaHCO₃ till pH 8 and then extracted with dichloromethane (40 mL×3). The combined organic phase was washed with brine (20 mL), dried over anhydrous sodium sulfate, filtered and concentrated in vacuum to give methyl 2-[3-[7-[[(3S,8S)-8-(hydroxymethyl)-1,2,3,5,6,7-hexahydropyrrolizin-3-yl]methyl]-2,7-diazaspiro[3.5]nonan-2-yl]isoxazol-5-yl]-3-methyl-butanoate (302 mg, 64%) as a yellow solid. MS (ESI) m/z: 461.2 [M+H]⁺.

Step 5: Preparation of tert-butyl 3-[7-[8-ethyl-3-(methoxymethoxy)-1-naphthyl]-8-fluoro-2-[[(3S,8S)-3-[[2-[5-(1-methoxycarbonyl-2-methyl-propyl)isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

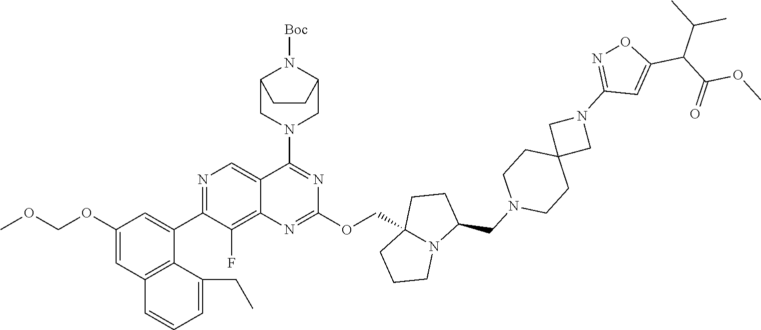

To a solution of methyl 2-[3-[7-[[(3S,8S)-8-(hydroxymethyl)-1,2,3,5,6,7-hexahydropyrrolizin-3-yl]methyl]-2,7-diazaspiro[3.5]nonan-2-yl]isoxazol-5-yl]-3-methyl-butanoate (302 mg, 0.656 mmol, 1.0 eq), 4 A MS (300 mg) and tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (427 mg, 0.656 mmol, 1.0 eq) in tetrahydrofuran (8 mL) was added t-BuONa (158 mg, 1.64 mmol, 2.5 eq) at 0° C. under N₂. The mixture was stirred at 0° C. for 23 min. Aq. HCl (0.2 N) was added to adjust the pH to 8, then extracted with dichloromethane (30 mL×4). The combined organic phase was washed with brine (20 mL), dried over anhydrous sodium sulfate, filtered, and concentrated in vacuum, the residue was purified by silica gel column (0˜4% then up to 10% methanol in dichloromethane; Gradient time: 11 min; Hold time: 25 min; Flow rate: 30 mL/min) to afford tert-butyl 3-[7-[8-ethyl-3-(methoxymethoxy)-1-naphthyl]-8-fluoro-2-[[(3S,8S)-3-[[2-[5-(1-methoxycarbonyl-2-methyl-propyl)isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (493 mg, 57%) as a yellow solid. MS (ESI) m/z: 516.9 [M/2+H]⁺.

Step 6: Preparation of 2-[3-[7-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]-2,7-diazaspiro[3.5]nonan-2-yl]isoxazol-5-yl]-3-methyl-butanoic acid

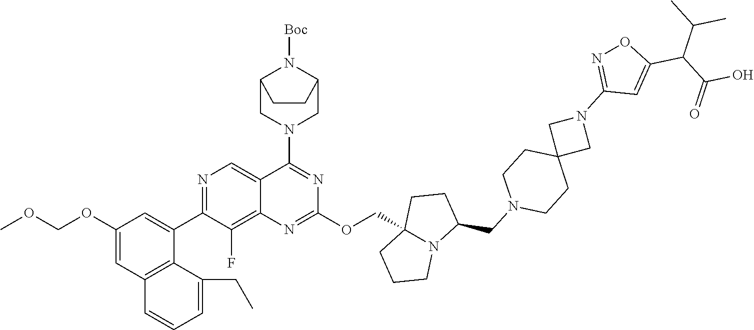 [SMILES: CCc1cccc2cc(OCOC)cc(-c3ncc4c(N5CC6CCC(C5)N6C(=O)OC(C)(C)C)nc(OC[C@@]56CCCN5[C@H](CN5CCC7(CC5)CN(c5cc(C(C(=O)O)C(C)C)on5)C7)CC6)nc4c3F)c12]

To a solution of tert-butyl 3-[7-[8-ethyl-3-(methoxymethoxy)-1-naphthyl]-8-fluoro-2-[[(3S,8S)-3-[[2-[5-(1-methoxycarbonyl-2-methyl-propyl)isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (493 mg, 0.48 mmol, 1.0 eq) in THF (6 mL) was added a solution of LiOH (100 mg, 2.39 mmol, 5.0 eq) in H₂O (2 mL). The mixture was stirred at 20° C. for 12 hours, then the mixture was stirred at 28° C. for 8 hours. Concentrated under reduced pressure, and the aqueous phase was acidified with aqueous HCl (2 N) till pH 4 and then extracted with dichloromethane/MeOH (100 mL×3, 10/1). The combined organic phase was washed with brine (20 mL), dried over anhydrous sodium sulfate, filtered, and concentrated in vacuum to afford 2-[3-[7-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]-2,7-diazaspiro[3.5]nonan-2-yl]isoxazol-5-yl]-3-methyl-butanoic acid (422 mg, 65%) as a yellow solid. MS (ESI) m/z: 509.5 [M/2+H]⁺.

Step 7: Preparation of tert-butyl 3-[7-[8-ethyl-3-(methoxymethoxy)-1-naphthyl]-8-fluoro-2-[[(3S,8S)-3-[[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate and tert-butyl 3-[7-[8-ethyl-3-(methoxymethoxy)-1-naphthyl]-8-fluoro-2-[[(3S,8S)-3-[[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

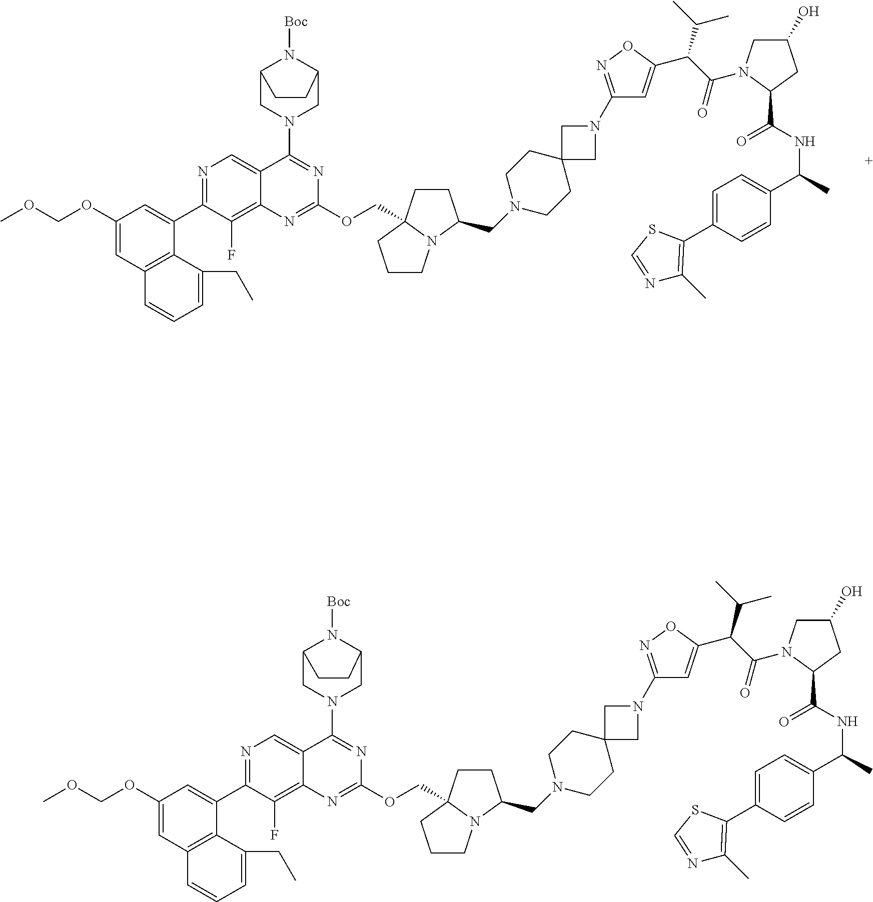

To a solution of 2-[3-[7-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]-2,7-diazaspiro[3.5]nonan-2-yl]isoxazol-5-yl]-3-methyl-butanoic acid (422 mg, 0.41 mmol, 1.0 eq) and (2S,4R)-4-hydroxy-N-[(1S)-1-[4-(4-methylthiazol-5-yl)phenyl]ethyl]pyrrolidine-2-carboxamide (210 mg, 0.5 mmol, 1.2 eq, HCl) in DMF (6 mL) was added DIEA (2.07 mmol, 361 μL, 5.0 eq) and HATU (173 mg, 0.46 mmol, 1.1 eq). The mixture was stirred at 20° C. for 12 hours. The mixture was added to water (20 mL) and stirred for a while then filtered, the filter cake was washed with water (10 mL), diluted with dichloromethane (50 mL), dried over anhydrous sodium sulfate, filtered and concentrated in vacuum. The residue was purified by flash column (0˜5% then up to 7% methanol (3N NH₃) in dichloromethane; Gradient time: 15 min; Hold time: 18 min; Flow rate: 30 mL/min), then further separated by SFC (column: DAICEL CHIRALPAK IE (250 mm*30 mm, 10 um); mobile phase: [ACN/EtOH (0.1% NH₃H₂O)]; B %: 60%-60%, min) to afford tert-butyl 3-[7-[8-ethyl-3-(methoxymethoxy)-1-naphthyl]-8-fluoro-2-[[(3S,8S)-3-[[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (190 mg, 0.127 mmol, 31%) as a yellow solid, and tert-butyl 3-[7-[8-ethyl-3-(methoxymethoxy)-1-naphthyl]-8-fluoro-2-[[(3S,8S)-3-[[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (201 mg, 34%) as a yellow solid. MS (ESI) m/z: 666.6 [M/2+H]⁺.

Step 8: Preparation of (2S,4R)-1-[(2R)-2-[3-(7-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide

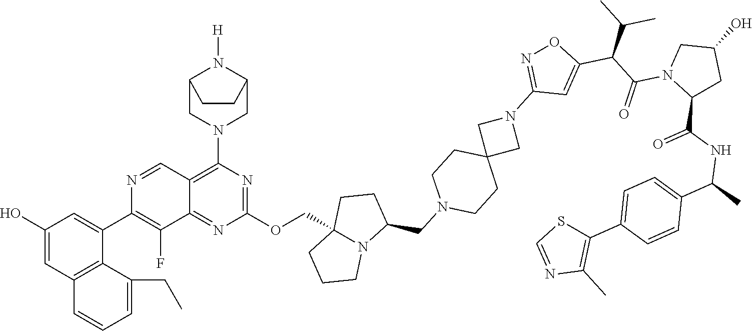 [SMILES: CCc1cccc2cc(O)cc(-c3ncc4c(N5CC6CCC(C5)N6)nc(OC[C@@]56CCCN5[C@H](CN5CCC7(CC5)CN(c5cc([C@H](C(=O)N8C[C@H](O)C[C@H]8C(=O)N[C@@H](C)c8ccc(-c9scnc9C)cc8)C(C)C)on5)C7)CC6)nc4c3F)c12]

To tert-butyl 3-[7-[8-ethyl-3-(methoxymethoxy)-1-naphthyl]-8-fluoro-2-[[(3S,8S)-3-[[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (120 mg, 0.09 mmol, 1.0 eq) in dichloromethane (6 mL) was added TFA (27.01 mmol, 2 mL) in one portion at 20° C. The mixture was stirred at 20° C. for 1 hour, then concentrated in vacuum 25° C. Adjusted the pH to 9 by addition of TEA. The residue was purified by prep-HPLC (column: Phenomenex C18 75*30 mm*3 um; mobile phase: [water (FA)-ACN]; B %: 8%-48%, 28 min) to afford (2S,4R)-1-[(2R)-2-[3-(7-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}-2,7-diazaspiro[3.5]nonan-2-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (68.3 mg, 62%) as a white solid. MS (ESI) m/z: 1188.0 [M+H]+; ¹H NMR (400 MHz, CD₃OD) δ 9.13 (d, J=3.6 Hz, 1H), 8.90-8.84 (m, 1H), 8.44 (s, 2H), 7.64 (d, J=8.0 Hz, 1H), 7.49-7.32 (m, 6H), 7.31-7.27 (m, 1H), 7.17 (d, J=7.2 Hz, 1H), 7.01 (d, J=2.4 Hz, 1H), 5.88-5.80 (m, 1H), 5.06-5.00 (m, 1H), 4.83-4.68 (m, 4H), 4.64-4.56 (m, 1H), 4.50 (t, J=8.0 Hz, 1H), 4.46-4.39 (m, 1H), 4.24-4.11 (m, 1H), 3.92-3.74 (m, 6H), 3.65-3.57 (m, 7H), 2.80-2.72 (m, 1H), 2.67-2.51 (m, 3H), 2.49-2.47 (m, 3H), 2.42-2.31 (m, 6H), 2.22-2.15 (m, 3H), 2.10-1.95 (m, 8H), 1.80 (d, J=16.4 Hz, 4H), 1.58-1.50 (m, 3H), 1.05 (d, J=6.4 Hz, 3H), 0.91-0.86 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (Compound 89)

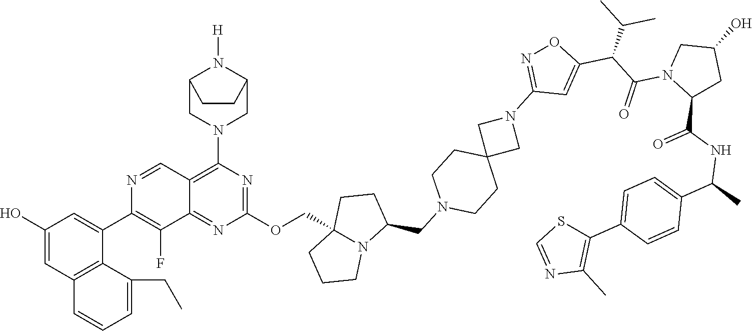 [SMILES: CCc1cccc2cc(O)cc(-c3ncc4c(N5CC6CCC(C5)N6)nc(OC[C@@]56CCCN5[C@H](CN5CCC7(CC5)CN(c5cc([C@@H](C(=O)N8C[C@H](O)C[C@H]8C(=O)N[C@@H](C)c8ccc(-c9scnc9C)cc8)C(C)C)on5)C7)CC6)nc4c3F)c12]

The title compound was prepared in an analogous manner to Compound 87 starting from tert-butyl 3-[7-[8-ethyl-3-(methoxymethoxy)-1-naphthyl]-8-fluoro-2-[[(3S,8S)-3-[[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,7-diazaspiro[3.5]nonan-7-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate, and purified by prep-HPLC (column: Phenomenex C18 75*30 mm*3 um; mobile phase: [water (FA)-ACN]; B %: 5%-45%, 28 min) to afford [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (69.8 mg, 63%) as a white solid. MS (ESI) m/z: 1188.0 [M+H]+; ¹H NMR (400 MHz, CD₃OD) δ 9.13 (d, J=6.4 Hz, 1H), 8.92-8.82 (m, 1H), 8.45 (s, 1H), 7.63 (t, J=6.8 Hz, 1H), 7.48-7.27 (m, 6H), 7.17 (d, J=6.4 Hz, 1H), 7.01 (s, 1H), 5.90-5.81 (m, 1H), 5.02-4.97 (m, 1H), 4.78-4.66 (m, 3H), 4.64-4.54 (m, 3H), 4.43 (s, 1H), 4.28-4.06 (m, 1H), 3.87-3.79 (m, 3H), 3.78-3.52 (m, 10H), 2.80-2.70 (m, 1H), 2.66-2.53 (m, 2H), 2.51 (d, J=2.1 Hz, 1H), 2.46 (s, 3H), 2.42-2.29 (m, 6H), 2.27-2.17 (m, 3H), 2.11-1.90 (m, 8H), 1.88-1.81 (m, 2H), 1.79-1.76 (m, 1H), 1.71 (s, 1H), 1.54-1.43 (m, 3H), 1.06 (d, J=6.8 Hz, 3H), 0.93-0.86 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (Compound 90)

Step 1: Preparation of methyl 2-[3-[(tert-butoxycarbonylamino)methyl]isoxazol-5-yl]-3-methyl-butanoate

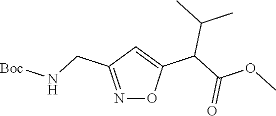 [SMILES: COC(=O)C(c1cc(CNC(=O)OC(C)(C)C)no1)C(C)C]

To a stirred solution of 2-[5-(1-methoxycarbonyl-2-methyl-propyl)isoxazol-3-yl]acetic acid (4.01 g, 16.6 mmol, 1 eq) in toluene (30 mL)/t-BuOH (32 mL) were added 4 A MS (4.0 g) and TEA (5.05 g, 49.9 mmol, 3 eq), the reaction mixture was stirred at 110° C. under N₂ for 0.5 hour. After cooling to 20° C., DPPA (6.86 g, 24.9 mmol, 5.40 mL, 1.5 eq) was added, and the reaction mixture was stirred at 110° C. under N₂ for 16 hours. The reaction mixture was filtered, the filtrate was poured into water (45 mL) and extracted with EtOAc (30 mL×3). The combined organic layer was washed with brine (30 mL×2), dried over anhydrous Na₂SO₄, filtered and concentrated under reduced pressure, the residue was purified by silica gel column (0˜8% tetrahydrofuran in petroleum ether; Gradient time: 15 min; Hold time: 10 min; Flow rate: 60 mL/min) to give methyl 2-[3-[(tert-butoxycarbonylamino)methyl]isoxazol-5-yl]-3-methyl-butanoate (771 mg, 15%) as a yellow oil. MS (ESI) m/z: 313.0 [M+H]⁺; ¹H NMR (CDCl₃, 400 MHz) δ 6.25 (s, 1H), 4.99 (brs, 1H), 4.39 (d, J=5.6 Hz, 2H), 3.74 (s, 3H), 3.63 (d, J=8.4 Hz, 1H), 2.46-2.32 (m, 1H), 1.47 (s, 9H), 1.01 (d, J=6.8 Hz, 3H), 0.91 (d, J=6.8 Hz, 3H).

Step 2: Preparation of methyl 2-[3-(formamidomethyl)isoxazol-5-yl]-3-methyl-butanoate

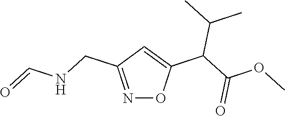 [SMILES: COC(=O)C(c1cc(CNC=O)no1)C(C)C]

A mixture of methyl 2-[3-[(tert-butoxycarbonylamino)methyl]isoxazol-5-yl]-3-methyl-butanoate (1.5 g, 4.80 mmol, 1 eq) in HCOOH (10 mL) was stirred at 90° C. for 48 hours. The reaction mixture was concentrated under reduced pressure, the residue was diluted with EtOAc (50 mL) and washed with saturated NaHCO₃ solution (20 mL×2), brine (20 mL×2), dried over anhydrous Na₂SO₄, filtered and concentrated under reduced pressure to give methyl 2-[3-(formamidomethyl)isoxazol-5-yl]-3-methyl-butanoate (2.51 g, 55% purity) as a brown oil, which was used in the next step directly. MS (ESI) m/z: 241.1 [M+H]⁺.

Step 3: Preparation of methyl2-[3-[[tert-butoxycarbonyl(methyl)amino]methyl]isoxazol-5-yl]-3-methyl-butanoate

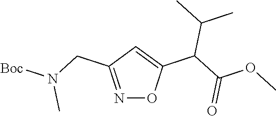 [SMILES: COC(=O)C(c1cc(CN(C)C(=O)OC(C)(C)C)no1)C(C)C]

To a stirred solution of methyl 2-[3-(formamidomethyl)isoxazol-5-yl]-3-methyl-butanoate (2.51 g, 5.72 mmol, 55% purity, 1 eq) in THF (20 mL) was added BH₃·THF (1 M, 23 mL, 4 eq) at 0° C., and the reaction mixture was stirred at 20° C. for 4 hours. The reaction solution was quenched by addition of MeOH (2.5 mL)/2N HCl (15 mL) and stirred at 70° C. for 1 hour. Then NaHCO₃ (2 M, 28.62 mL, 10 eq) and Boc₂O (3.75 g, 17.17 mmol, 3 eq) were added, and the reaction mixture was stirred at 50° C. for 2 hours. The reaction mixture was quenched by water (20 mL) and extracted with EtOAc (20 mL×3). The combined organic layer was washed with brine (20 mL), dried over anhydrous Na₂SO₄, filtered and concentrated under reduced pressure. The residue was purified by column chromatography (0˜5% tetrahydrofuran in petroleum ether; Gradient time: 10 min; Hold time: 15 min; Flow rate: 50 mL/min) to give methyl2-[3-[[tert-butoxycarbonyl(methyl)amino]methyl]isoxazol-5-yl]-3-methyl-butanoate (1.10 g, 59%) as yellow oil. MS (ESI) m/z: 327.1 [M+H]⁺; ¹H NMR (CDCl₃, 400 MHz) δ 6.21 (s, 1H), 4.56-4.35 (m, 2H), 3.74 (s, 3H), 3.63 (d, J=8.8 Hz, 1H), 2.96-2.78 (m, 3H), 2.46-2.31 (m, 1H), 1.49 (s, 9H), 1.01 (d, J=6.4 Hz, 3H), 0.90 (d, J=6.8 Hz, 3H).

Step 4: Preparation of 2-[3-[[tert-butoxycarbonyl(methyl)amino]methyl]isoxazol-5-yl]-3-methyl-butanoic acid

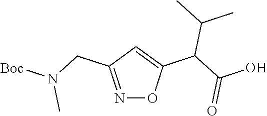 [SMILES: CC(C)C(C(=O)O)c1cc(CN(C)C(=O)OC(C)(C)C)no1]

To a stirred solution of methyl 2-[3-[[tert-butoxycarbonyl(methyl)amino]methyl]isoxazol-5-yl]-3-methyl-butanoate (1.10 g, 3.37 mmol, 1 eq) in MeOH (4 mL)/THF (4 mL) was added a solution of LiOH (354 mg, 8.43 mmol, 2.5 eq) in water (4 mL), the reaction mixture was stirred at 20° C. for 3 hours, then concentrated under reduced pressure to remove methanol and tetrahydrofuran. The pH of the resulting mixture was adjusted to 5 with 2 M aq. HCl and extracted with dichloromethane (15 mL×3). The combined organic layer was washed with brine (20 mL), dried over anhydrous Na₂SO₄, filtered and concentrated reduced under pressure to give 2-[3-[[tert-butoxycarbonyl(methyl)amino]methyl]isoxazol-5-yl]-3-methyl-butanoic acid (1.01 g, 95%) as a red oil, which was used in the next step directly. MS (ESI) m/z: 313.1 [M+H]⁺.

Step 5: Preparation of tert-butyl N-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]-N-methyl-carbamate

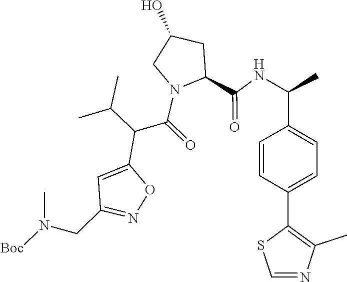 [SMILES: Cc1ncsc1-c1ccc([C@H](C)NC(=O)[C@@H]2C[C@@H](O)CN2C(=O)C(c2cc(CN(C)C(=O)OC(C)(C)C)no2)C(C)C)cc1]

To a stirred solution of 2-[3-[[tert-butoxycarbonyl(methyl)amino]methyl]isoxazol-5-yl]-3-methyl-butanoic acid (1.01 g, 3.20 mmol, 1 eq) in CH₂Cl₂ (20 mL) were added DIEA (2.07 g, 16.0 mmol, 2.79 mL, 5 eq) and HATU (1.46 g, 3.84 mmol, 1.2 eq), the reaction mixture was stirred at 20° C. for 10 min. Then (2S,4R)-4-hydroxy-N-[(1S)-1-[4-(4-methylthiazol-5-yl)phenyl]ethyl]pyrrolidine-2-carboxamide (1.52 g, 1.1 eq, HCl) was added, and the reaction mixture was stirred at 20° C. for 1 hour. The reaction was poured into water (20 mL) and the layers were separated. The aqueous layer was extracted with CH₂Cl₂ (20 mL). The combined organic layer was washed with brine (20 mL), dried over anhydrous Na₂SO₄, filtered and concentrated under reduced pressure. The crude product was purified by Biotage® combi flash (Column: 20 g Biotage® Silica Flash column; Eluent: gradient 0˜100% ethyl acetate in petroleum ether; Gradient time: 20 min; Hold time: 15 min; Flow rate: 70 mL/min) to give tert-butyl N-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]-N-methyl-carbamate (1.90 g, 95%) as a yellow solid. MS (ESI) m/z: 626.2 [M+H]⁺.

Step 6: Preparation of tert-butyl N-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]-N-methyl-carbamate and tert-butyl N-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]-N-methyl-carbamate

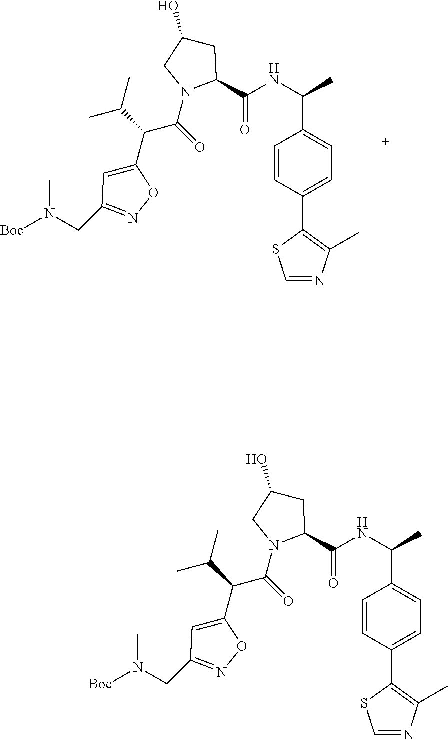 [SMILES: Cc1ncsc1-c1ccc([C@H](C)NC(=O)[C@@H]2C[C@@H](O)CN2C(=O)[C@H](c2cc(CN(C)C(=O)OC(C)(C)C)no2)C(C)C)cc1 ; Cc1ncsc1-c1ccc([C@H](C)NC(=O)[C@@H]2C[C@@H](O)CN2C(=O)[C@@H](c2cc(CN(C)C(=O)OC(C)(C)C)no2)C(C)C)cc1]

Tert-butyl N-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]-N-methyl-carbamate (1.90 g, 3.04 mmol) was separated by SFC (column: DAICEL CHIRALPAK IA (250 mm*30 mm, 10 um); mobile phase: [ACN/EtOH (0.1% NH₃H₂O)]; B %: 30%-30%) to give tert-butyl N-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]-N-methyl-carbamate (911 mg, 48%) as a yellow solid and tert-butyl N-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]-N-methyl-carbamate (595 mg, 31%) as a yellow solid. MS (ESI) m/z: 626.3 [M+H]⁺.

Step 7: Preparation of (2S,4R)-4-hydroxy-1-[(2R)-3-methyl-2-[3-(methylaminomethyl)isoxazol-5-yl]butanoyl]-N-[(1S)-1-[4-(4-methylthiazol-5-yl)phenyl]ethyl]pyrrolidine-2-carboxamide

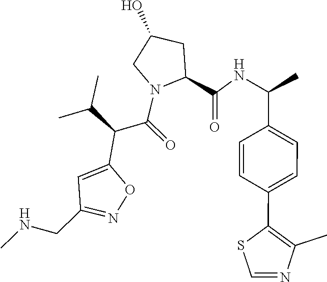

A mixture of tert-butyl N-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]-N-methyl-carbamate (300 mg, 0.48 mmol, 1 eq) in CH₂Cl₂ (2 mL)/HCl/dioxane (2 mL, 4 M) was stirred at 20° C. for 1 hour. The reaction mixture was concentrated at 35° C. under reduced pressure to give (2S,4R)-4-hydroxy-1-[(2R)-3-methyl-2-[3-(methylaminomethyl)isoxazol-5-yl]butanoyl]-N-[(1S)-1-[4-(4-methylthiazol-5-yl)phenyl]ethyl]pyrrolidine-2-carboxamide (266 mg, crude) as a yellow solid. MS (ESI) m/z: 526.2 [M+H]⁺.

Step 8: Preparation of tert-butyl 3-[7-[8-ethyl-3-(methoxymethoxy)-1-naphthyl]-8-fluoro-2-[[(3S,8S)-3-[[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl-methyl-carbamoyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

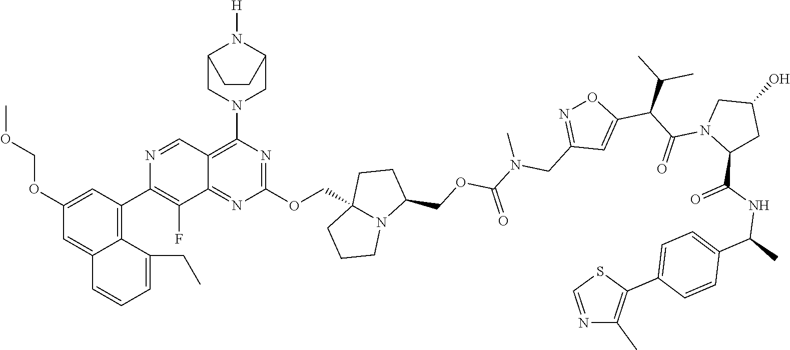 [SMILES: CCc1cccc2cc(OCOC)cc(-c3ncc4c(N5CC6CCC(C5)N6)nc(OC[C@@]56CCCN5[C@H](COC(=O)N(C)Cc5cc([C@H](C(=O)N7C[C@H](O)C[C@H]7C(=O)N[C@@H](C)c7ccc(-c8scnc8C)cc7)C(C)C)on5)CC6)nc4c3F)c12]

To a solution of (4-nitrophenyl) carbonochloridate (46.0 mg, 0.23 mmol, 1.7 eq) in THF (3 mL) were added TEA (1.35 mmol, 187.0 uL, 10.0 eq), DMAP (2.0 mg, 0.013 mmol, 0.1 eq) and tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (100.0 mg, 0.14 mmol, 1.0 eq). The reaction mixture was stirred at 40° C. for 15 hr. Then (2S,4R)-4-hydroxy-1-[(2R)-3-methyl-2-[3-(methylaminomethyl)isoxazol-5-yl]butanoyl]-N-[(1S)-1-[4-(4-methylthiazol-5-yl)phenyl]ethyl]pyrrolidine-2-carboxamide (76.0 mg, 0.14 mmol, 1.0 eq, HCl) was added to the mixture, and the reaction mixture was stirred at 40° C. for 1 hr. The mixture was concentrated, the residue was purified by Biotage® combi flash (Column: 4 g Biotage® Silica Flash column; Eluent: gradient 0˜7% methanol in dichloromethane; Gradient time: 10 min; Hold time: 12 min; Flow rate: 30 mL/min) to afford tert-butyl 3-[7-[8-ethyl-3-(methoxymethoxy)-1-naphthyl]-8-fluoro-2-[[(3S,8S)-3-[[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl-methyl-carbamoyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (130 mg, 59%) as a yellow solid. MS (ESI) m/z: 1294.4 [M+H]⁺.

Step 9: Preparation of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate

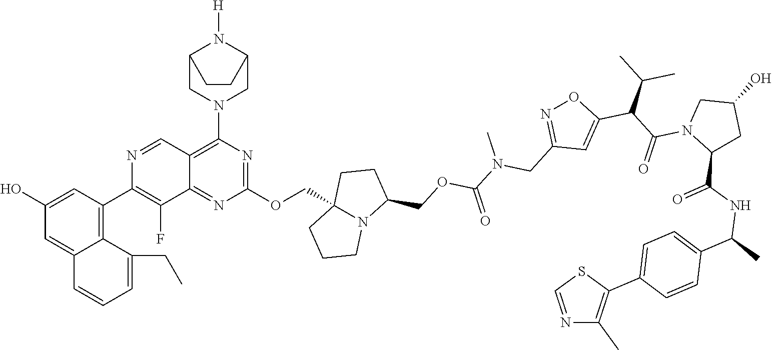

A solution of tert-butyl 3-[7-[8-ethyl-3-(methoxymethoxy)-1-naphthyl]-8-fluoro-2-[[(3S,8S)-3-[[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl-methyl-carbamoyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (130 mg, 0.08 mmol, 1.0 eq) in CH₂Cl₂ (2 mL) and 4 M HCl/dioxane (2 mL) was stirred at 25° C. for 0.5 hour. To the mixture was added petroleum ether (30 mL) and stirred at 25° C. for 0.5 hour. Then the mixture was put to static settlement for 20 min and the liquid supernatant was removed. The residue was concentrated, purified by prep-HPLC (column: Phenomenex C18 75×30 mm×3 um; mobile phase: [water (FA)-ACN]; B %: 5%-35%, 25 min) to afford [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (50.1 mg, 53%, FA) as a white solid. MS (ESI) m/z: 1150.9 [M+1]⁺; 1H NMR (400 MHz, CD₃OD) δ 9.16-9.06 (m, 1H), 8.89-8.83 (m, 1H), 8.45 (s, 1H), 7.63 (d, J=8.0 Hz, 1H), 7.46-7.34 (m, 5H), 7.30 (d, J=2.4 Hz, 1H), 7.16 (d, J=7.2 Hz, 1H), 7.05-6.96 (m, 1H), 6.40-6.23 (m, 1H), 5.03 (q, J=7.2 Hz, 1H), 4.81-4.41 (m, 10H), 4.25-4.08 (m, 1H), 3.93-3.45 (m, 8H), 3.02-2.92 (m, 3H), 2.49-1.84 (m, 21H), 1.61-1.47 (m, 3H), 1.09-1.01 (m, 3H), 0.92-0.79 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (Compound 91)

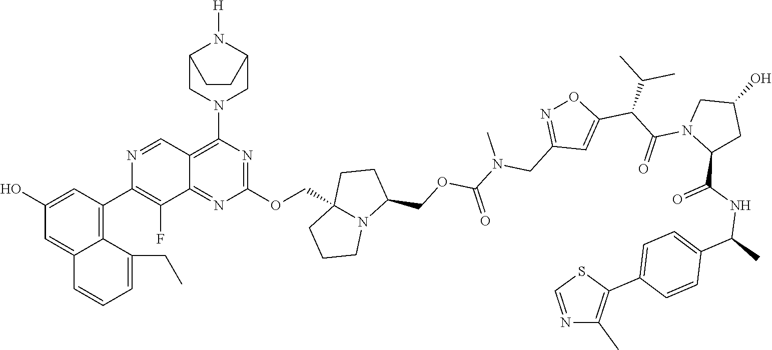 [SMILES: CCc1cccc2cc(O)cc(-c3ncc4c(N5CC6CCC(C5)N6)nc(OC[C@@]56CCCN5[C@H](COC(=O)N(C)Cc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 90 starting from (2S,4R)-4-hydroxy-1-[(2S)-3-methyl-2-[3-(methylaminomethyl)isoxazol-5-yl]butanoyl]-N-[(1S)-1-[4-(4-methylthiazol-5-yl)phenyl]ethyl]pyrrolidine-2-carboxamide, and purified by prep-HPLC (column: Phenomenex C18 75*30 mm*3 um; mobile phase: [water (FA)-ACN]; B %: 5%-35%, 25 min). Pure fraction was lyophilized to afford as a white solid. MS (ESI) m/z: 1150.9 [M+1]⁺; 1H NMR (400 MHz, CD₃OD) δ 9.10 (s, 1H), 8.91-8.80 (m, 1H), 8.46 (s, 1H), 7.63 (d, J=8.4 Hz, 1H), 7.48-7.26 (m, 6H), 7.16 (d, J=6.4 Hz, 1H), 7.00 (t, J=2.8 Hz, 1H), 6.40-6.24 (m, 1H), 4.93 (d, J=6.8 Hz, 1H), 4.78-4.63 (m, 4H), 4.61-4.48 (m, 5H), 4.44-4.33 (m, 2H), 4.24-4.09 (m, 1H), 3.93-3.41 (m, 8H), 2.96 (d, J=3.2 Hz, 3H), 2.49-2.42 (m, 3H), 2.41-2.14 (m, 7H), 2.14-1.80 (m, 10H), 1.59-1.43 (m, 3H), 1.03 (d, J=6.0 Hz, 3H), 0.91-0.73 (m, 6H).

Exemplary Synthesis of [(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (Compound 92)

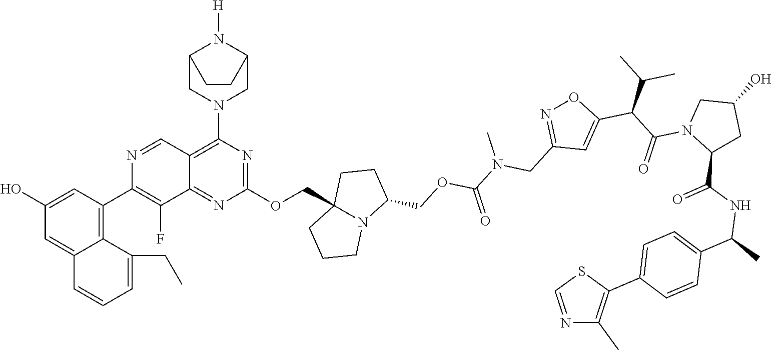 [SMILES: CCc1cccc2cc(O)cc(-c3ncc4c(N5CC6CCC(C5)N6)nc(OC[C@]56CCCN5[C@@H](COC(=O)N(C)Cc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 90 starting from tert-butyl 3-[7-[8-ethyl-3-(methoxymethoxy)-1-naphthyl]-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate, and purified by prep-HPLC (column: Welch Xtimate C18 150*25 mm*5 um; mobile phase: [water (FA)-ACN]; B %: 5%-35%, 25 min) to afford [(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (40.2 mg, 42%, FA) as a white solid. MS (ESI) m/z: 1150.9 [M+H]⁺; ¹H NMR (CD₃OD, 400 MHz) δ 9.13-9.07 (m, 1H), 8.90-8.85 (m, 1H), 8.48 (s, 1H), 7.63 (d, J=8.0 Hz, 1H), 7.46-7.34 (m, 5H), 7.29 (d, J=2.4 Hz, 1H), 7.16 (d, J=6.8 Hz, 1H), 7.03-6.98 (m, 1H), 6.39-6.25 (m, 1H), 5.05-5.01 (m, 1H), 4.78-4.60 (m, 4H), 4.59-4.49 (m, 4H), 4.25-4.07 (m, 1H), 3.88-3.69 (m, 6H), 3.64-3.57 (m, 1H), 3.48-3.40 (m, 2H), 3.03-2.94 (m, 3H), 2.50-2.44 (m, 3H), 2.43-1.78 (m, 19H), 1.61-1.46 (m, 3H), 1.09-0.99 (m, 3H), 0.92-0.77 (m, 6H).

Exemplary Synthesis of [(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (Compound 93)

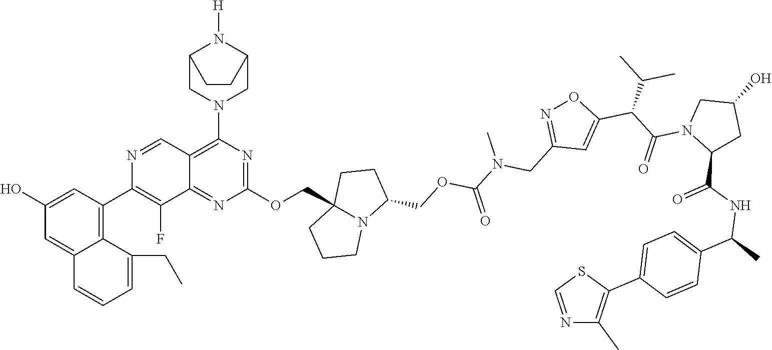 [SMILES: CCc1cccc2cc(O)cc(-c3ncc4c(N5CC6CCC(C5)N6)nc(OC[C@]56CCCN5[C@@H](COC(=O)N(C)Cc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 90 starting from tert-butyl 3-[7-[8-ethyl-3-(methoxymethoxy)-1-naphthyl]-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate, and purified by prep-HPLC (column: Welch Xtimate C18 150*25 mm*5 um; mobile phase: [water (FA)-ACN]; B %: 5%-35%, 25 min) to afford [(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl)-N-methylcarbamate (34.6 mg, 44%, FA) as a white solid. MS (ESI) m/z: 1150.9 [M+H]⁺; ¹H NMR (CD₃OD, 400 MHz) δ 9.10 (s, 1H), 8.90-8.83 (m, 1H), 8.47 (s, 1H), 7.63 (d, J=8.8 Hz, 1H), 7.47-7.32 (m, 5H), 7.29 (d, J=2.4 Hz, 1H), 7.16 (d, J=7.2 Hz, 1H), 7.03-6.97 (m, 1H), 6.39-6.28 (m, 1H), 4.98-4.96 (m, 1H), 4.77-4.60 (m, 6H), 4.45-4.36 (m, 2H), 4.23-4.05 (m, 1H), 3.92-3.68 (m, 6H), 3.66-3.42 (m, 3H), 2.96 (s, 3H), 2.49-2.44 (m, 3H), 2.43-1.79 (m, 19H), 1.60-1.44 (m, 3H), 1.08-1.00 (m, 3H), 0.92-0.79 (m, 6H).

Exemplary Synthesis of (2S,4R)-1-[(2R)-2-[3-(4-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperazin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 94)

Step 1: Preparation of methyl 3-methyl-2-(3-piperazin-1-ylisoxazol-5-yl) butanoate

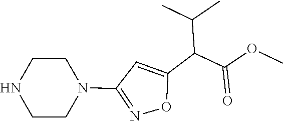 [SMILES: COC(=O)C(c1cc(N2CCNCC2)no1)C(C)C]

To a solution of tert-butyl 4-[5-(1-methoxycarbonyl-2-methyl-propyl)isoxazol-3-yl]piperazine-1-carboxylate (1.0 g, 2.72 mmol, 1.0 eq) in CH₂Cl₂ (6 mL) was added HCl/dioxane (4 M, 6.0 mL, 8.8 eq), and the reaction mixture was stirred at 20° C. for 2 hours, then concentrated in vacuum. Slowly adjusted the pH to 8˜9 by sat. aq. NaHCO₃ solution and extracted with dichloromethane (40 mL×3). The combined organic layers were washed with brine (30 mL), dried over anhydrous Na₂SO₄, filtered, and concentrated in vacuum to afford methyl 3-methyl-2-(3-piperazin-1-ylisoxazol-5-yl) butanoate (730 mg, crude) as a yellow oil, which was used in the next step without further purification. MS (ESI) m/z: 268.0 [M+1]⁺

Step 2: Preparation of methyl 2-[3-[4-[[(3S,8S)-8-[[tert-butyl(diphenyl)silyl]oxymethyl]-1,2,3,5,6,7-hexahydropyrrolizin-3-yl]methyl]piperazin-1-yl]isoxazol-5-yl]-3-methyl-butanoate

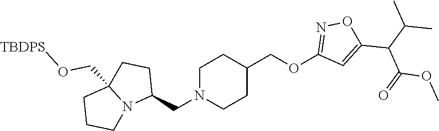

To a solution of methyl 3-methyl-2-(3-piperazin-1-ylisoxazol-5-yl) butanoate (369 mg, 1.38 mmol, 1.0 eq) and (3S,8S)-8-[[tert-butyl(diphenyl)silyl]oxymethyl]-1,2,3,5,6,7-hexahydropyrrolizine-3-carbaldehyde (676 mg, 1.66 mmol, 1.2 eq) in CH₂Cl₂ (10 mL) was added NaBH(OAc)₃ (644 mg, 3.04 mmol, 2.2 eq), and the reaction mixture was stirred at 20° C. for 12 hours. The reaction mixture was quenched by sat. NaHCO₃ (15 mL), the aqueous phase was extracted with dichloromethane (30 mL×4). The combined organic phase was washed with brine (20 mL), dried over anhydrous Na₂SO₄, filtered and concentrated in vacuum. The residue was purified by flash chromatography (0˜5% methanol in dichloromethane) to afford methyl 2-[3-[4-[[(3S,8S)-8-[[tert-butyl(diphenyl)silyl]oxymethyl]-1,2,3,5,6,7-hexahydropyrrolizin-3-yl]methyl]piperazin-1-yl]isoxazol-5-yl]-3-methyl-butanoate (670 mg, 63%) as a yellow oil. MS (ESI) m/z: 659.3 [M+1]⁺.

Step 3: Preparation of methyl 2-[3-[4-[[(3S,8S)-8-(hydroxymethyl)-1,2,3,5,6,7-hexahydropyrrolizin-3-yl]methyl]piperazin-1-yl]isoxazol-5-yl]-3-methyl-butanoate

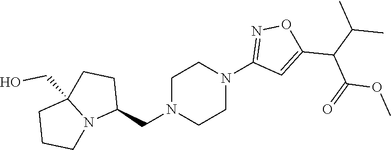 [SMILES: COC(=O)C(c1cc(N2CCN(C[C@@H]3CC[C@]4(CO)CCCN34)CC2)no1)C(C)C]

To a solution of methyl 2-[3-[4-[[(3S,8S)-8-[[tert-butyl(diphenyl)silyl]oxymethyl]-1,2,3,5,6,7-hexahydropyrrolizin-3-yl]methyl]piperazin-1-yl]isoxazol-5-yl]-3-methyl-butanoate (500 mg, 0.76 mmol, 1.0 eq) in CH₂Cl₂ (10 mL) was added N,N-diethylethanamine trihydrofluoride (1.83 g, 11.38 mmol, 15.0 eq), and the reaction mixture was stirred at 20° C. for 1 hour. The mixture was concentrated under reduced pressure, then diluted with water (15 mL), and extracted with MTBE (20 mL×3). The aqueous phase was basified with sat. NaHCO₃ until pH 8 and then extracted with dichloromethane (30 mL×4). The combined organic phase was washed with brine (20 mL), dried over anhydrous Na₂SO₄, filtered, and concentrated in vacuum (at 15° C.) to afford methyl 2-[3-[4-[[(3S,8S)-8-(hydroxymethyl)-1,2,3,5,6,7-hexahydropyrrolizin-3-yl]methyl]piperazin-1-yl]isoxazol-5-yl]-3-methyl-butanoate (300 mg, crude) as a yellow solid. MS (ESI) m/z: 421.1 [M+1]⁺.

Step 4: Preparation of tert-butyl 3-[7-[8-ethyl-3-(methoxymethoxy)-1-naphthyl]-8-fluoro-2-[[(3S,8S)-3-[[4-[5-(1-methoxycarbonyl-2-methyl-propyl)isoxazol-3-yl]piperazin-1-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

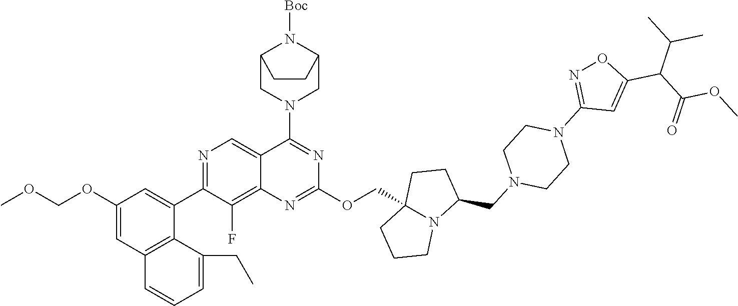

To a solution of tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (348 mg, 0.53 mmol, 1.0 eq), methyl 2-[3-[4-[[(3S,8S)-8-(hydroxymethyl)-1,2,3,5,6,7-hexahydropyrrolizin-3-yl]methyl]piperazin-1-yl]isoxazol-5-yl]-3-methyl-butanoate (300 mg, 534 mmol, 1.0 eq) and 4 A MS (300 mg, 0.53 mmol, 1.0 eq) in dioxane (6 mL) was added t-BuONa (154 mg, 1.60 mmol, 3.0 eq) under 0° C., and the reaction mixture was stirred at 10° C. for 20 minutes. The reaction mixture was quenched with HCl (2 M) to pH 8 and extracted with dichloromethane (30 mL×3). The organic layers were combined, dried over Na₂SO₄, filtered, and concentrated under reduced pressure. The residue was purified by flash chromatography (0˜5% methanol in dichloromethane) to afford tert-butyl 3-[7-[8-ethyl-3-(methoxymethoxy)-1-naphthyl]-8-fluoro-2-[[(3S,8S)-3-[[4-[5-(1-methoxycarbonyl-2-methyl-propyl)isoxazol-3-yl]piperazin-1-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (310 mg, 54%) as a yellow solid. MS (ESI) m/z: 992.3 [M+1]⁺.

Step 5: Preparation of 2-[3-[4-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]piperazin-1-yl]isoxazol-5-yl]-3-methyl-butanoic acid

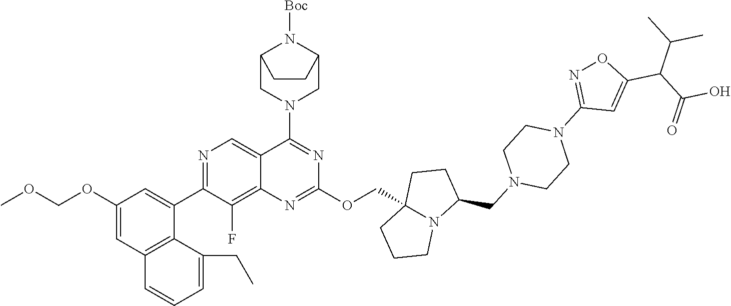 [SMILES: CCc1cccc2cc(OCOC)cc(-c3ncc4c(N5CC6CCC(C5)N6C(=O)OC(C)(C)C)nc(OC[C@@]56CCCN5[C@H](CN5CCN(c7cc(C(C(=O)O)C(C)C)on7)CC5)CC6)nc4c3F)c12]

To a solution of tert-butyl 3-[7-[8-ethyl-3-(methoxymethoxy)-1-naphthyl]-8-fluoro-2-[[(3S,8S)-3-[[4-[5-(1-methoxycarbonyl-2-methyl-propyl)isoxazol-3-yl]piperazin-1-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (310 mg, 0.29 mmol, 1.0 eq) in THF (3 mL) and H₂O (3 mL) was added LiOH·H₂O (49 mg, 1.16 mmol, 4.0 eq), and the reaction mixture was stirred at 20° C. for 12 hours. The reaction mixture was diluted with water (10 mL), slowly adjusted the pH to 5˜6 by HCl (2 M) and extracted with dichloromethane (20 mL×3). The combined organic layers were washed with brine (15 mL), dried over anhydrous Na₂SO₄, filtered, and concentrated in vacuum to afford 2-[3-[4-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]piperazin-1-yl]isoxazol-5-yl]-3-methyl-butanoic acid (284 mg, crude) as a yellow solid, which was used in the next step without further purification. MS (ESI) m/z: 978.3 [M+1]⁺.

Step 6: Preparation of (1R,5S)-tert-butyl 3-(7-(8-ethyl-3-(methoxymethoxy)naphthalen-1-yl)-8-fluoro-2-(((3S,7aS)-3-((4-(5-(1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperazin-1-yl)methyl) hexahydro-1H-pyrrolizin-7a-yl)methoxy)pyrido[4,3-d]pyrimidin-4-yl)-3,8-diazabicyclo[3.2.1]octane-8-carboxylate

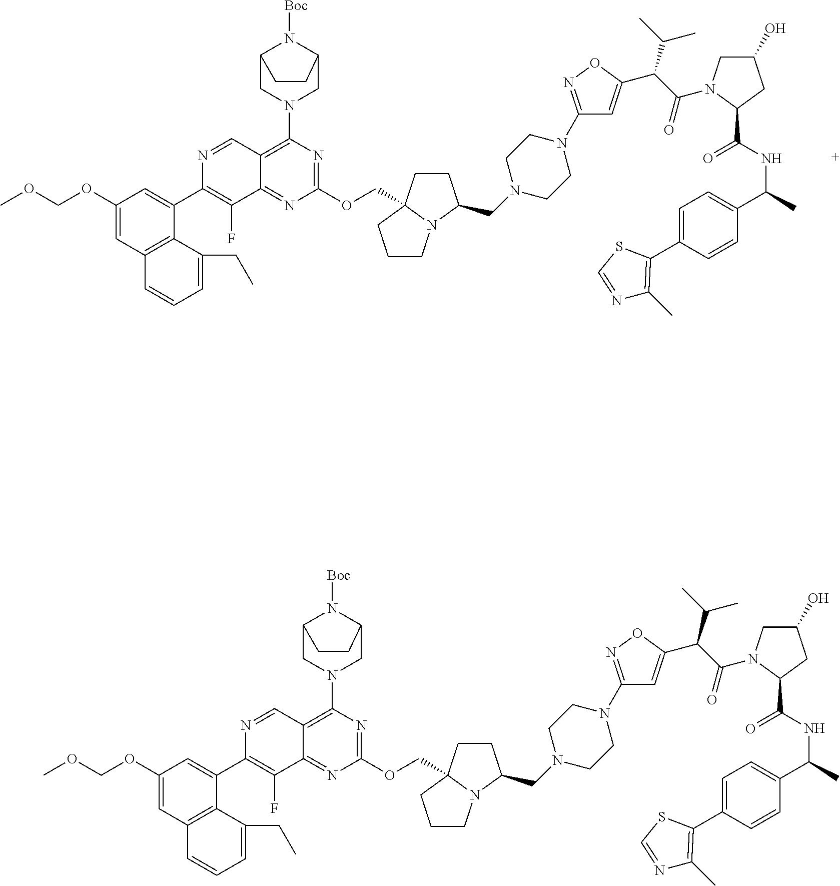

To a solution of 2-[3-[4-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]piperazin-1-yl]isoxazol-5-yl]-3-methyl-butanoic acid (284 mg, 0.29 mmol, 1.0 eq) and (2S,4R)-4-hydroxy-N-[(1S)-1-[4-(4-methylthiazol-5-yl)phenyl]ethyl]pyrrolidine-2-carboxamide (135 mg, 0.32 mmol, 1.1 eq, HCl) in DMF (4 mL) were added DIEA (0.253 mL, 1.45 mmol, 5.0 eq) and HATU (121 mg, 0.32 mmol, 1.1 eq), the reaction mixture was stirred at 20° C. for 1 hour. The reaction mixture was quenched by H₂O (10 mL), filtered, the filter cake was washed with water (10 mL×2). The filtrate was extracted with dichloromethane (20 mL×3). The organic phase was dried over anhydrous Na₂SO₄, filtered, and concentrated in vacuum. The residue was purified by flash chromatography (0˜10% methanol in dichloromethane), then further purified by SFC ([ACN/EtOH (0.1% NH₃H₂O)]; B %: 100%-100%, Gradient time: 10 min; column: DAICEL CHIRALPAK IE (50 mm*250 mm, 10 um; Flow Rate: 80 mL/min) to afford tert-butyl 3-[7-[8-ethyl-3-(methoxymethoxy)-1-naphthyl]-8-fluoro-2-[[(3S,8S)-3-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (120 mg, 28% yield) as a yellow solid, MS (ESI) m/z: 1291.4 [M+1]⁺; and tert-butyl 3-[7-[8-ethyl-3-(methoxymethoxy)-1-naphthyl]-8-fluoro-2-[[(3S,8S)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (110 mg, 28%) as a yellow solid. MS (ESI) m/z: 1291.4 [M+1]⁺.

Step 7: Preparation of (2S,4R)-1-[(2R)-2-[3-(4-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperazin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide

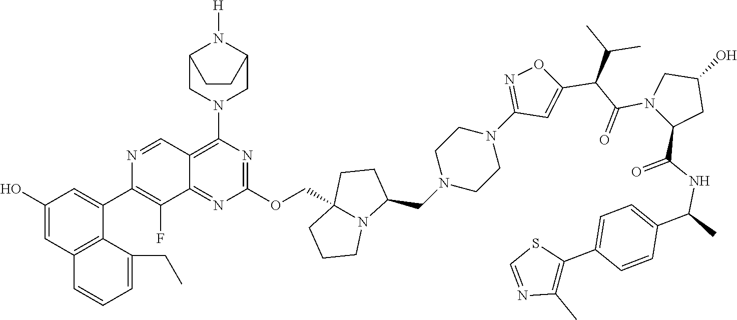 [SMILES: CCc1cccc2cc(O)cc(-c3ncc4c(N5CC6CCC(C5)N6)nc(OC[C@@]56CCCN5[C@H](CN5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-[8-ethyl-3-(methoxymethoxy)-1-naphthyl]-8-fluoro-2-[[(3S,8S)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (110 mg, 0.08 mmol, 1.0 eq) in CH₂Cl₂ (2.2 mL) was added HCl/dioxane (4 M, 2.2 mL, 103.3 eq), and the reaction mixture was stirred at 20° C. for 0.5 hours. The reaction mixture was diluted with petroleum ether (20 mL) and filtered, the filter cake was dissolved in dichloromethane (20 mL, 0.05% TEA in dichloromethane) and concentrated in vacuum. The residue was purified by pre-HPLC (condition: [water (FA)-ACN]; B %: 10%-50%, gradient time: 25 min; hold time: 4 min; column: phenomenex C18 75×30 mm×3 um; flow rate: 25 mL/min) to afford (2S,4R)-1-[(2R)-2-[3-(4-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperazin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (61.8 mg, 60%, FA) as a white solid. MS (ESI) m/z: 1147.4 [M+1]⁺; 1H NMR (400 MHz, CD₃OD) δ 9.17-9.08 (m, 1H), 8.90-8.83 (m, 1H), 8.45 (s, 2H), 7.64 (d, J=8.4 Hz, 1H), 7.46-7.33 (m, 5H), 7.31-7.27 (m, 1H), 7.19-7.14 (m, 1H), 7.03-6.99 (m, 1H), 6.15-5.98 (m, 1H), 5.07-4.98 (m, 1H), 4.77-4.70 (m, 2H), 4.64-4.58 (m, 1H), 4.50 (t, J=8.0 Hz, 1H), 4.46-4.38 (m, 1H), 4.31-4.16 (m, 1H), 4.04 (d, J=10.8 Hz, 2H), 3.98-3.80 (m, 3H), 3.66-3.45 (m, 3H), 3.39-3.32 (m, 1H), 3.28-3.17 (m, 4H), 2.88-2.72 (m, 3H), 2.67-2.60 (m, 1H), 2.53 (dd, J=4.8, 10.4 Hz, 2H), 2.49-2.45 (m, 3H), 2.44-2.32 (m, 4H), 2.31 (s, 10H), 2.02-1.90 (m, 4H), 1.60-1.50 (m, 3H), 1.07-1.02 (m, 3H), 0.92-0.85 (m, 6H).

Exemplary Synthesis of (2S,4R)-1-[(2S)-2-[3-(4-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperazin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 95)

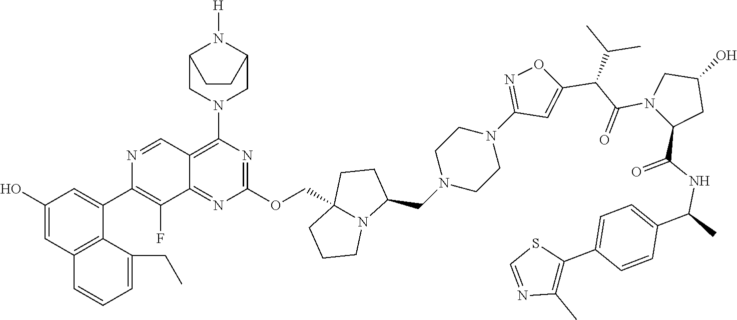 [SMILES: CCc1cccc2cc(O)cc(-c3ncc4c(N5CC6CCC(C5)N6)nc(OC[C@@]56CCCN5[C@H](CN5CCN(c7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 94 starting from tert-butyl 3-[7-[8-ethyl-3-(methoxymethoxy)-1-naphthyl]-8-fluoro-2-[[(3S,8S)-3-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate, and purified by prep-HPLC (condition: [water (FA)-ACN]; B %: 10%-50%, gradient time: 25 min; column: Welch Xtimate C18 150*25 mm*5 um; flow rate: 25 mL/min) to afford (2S,4R)-1-[(2S)-2-[3-(4-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperazin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (45.5 mg, 46%) as a white solid. MS (ESI) m/z: 1148.1 [M+1]⁺; 1H NMR (400 MHz, CD₃OD) δ 9.12 (d, J=6.0 Hz, 1H), 8.92-8.82 (m, 1H), 8.43 (s, 2H), 7.63 (dd, J=4.4, 7.6 Hz, 1H), 7.48-7.38 (m, 1H), 7.38-7.27 (m, 5H), 7.16 (d, J=6.8 Hz, 1H), 7.00 (s, 1H), 6.14-6.03 (m, 1H), 4.97-4.94 (m, 1H), 4.77-4.70 (m, 2H), 4.64-4.53 (m, 2H), 4.48-4.34 (m, 1H), 4.30-4.14 (m, 1H), 3.97 (d, J=7.6 Hz, 2H), 3.93-3.80 (m, 2H), 3.76-3.59 (m, 4H), 3.38-3.34 (m, 1H), 3.35 (s, 1H), 3.26-3.21 (m, 2H), 3.21-3.11 (m, 2H), 2.87-2.69 (m, 3H), 2.61 (dd, J=5.6, 9.7 Hz, 1H), 2.48 (s, 2H), 2.45 (s, 3H), 2.36 (dd, J=8.0, 14.8 Hz, 3H), 2.32-2.14 (m, 5H), 2.13-1.90 (m, 9H), 1.46 (d, J=7.2 Hz, 3H), 1.05 (d, J=6.8 Hz, 3H), 0.94-0.83 (m, 6H).

Exemplary Synthesis of (2S,4R)-1-[(2R)-2-{3-[(1-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 96)

Step 1: Preparation of tert-butyl 4-[[5-(1-methoxycarbonyl-2-methyl-propyl)isoxazol-3-yl]oxymethyl]piperidine-1-carboxylate

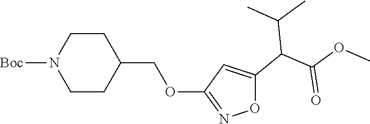 [SMILES: COC(=O)C(c1cc(OCC2CCN(C(=O)OC(C)(C)C)CC2)no1)C(C)C]

To a stirred solution of methyl 2-(3-hydroxyisoxazol-5-yl)-3-methyl-butanoate (1.5 g, 7.53 mmol, 1.0 eq) in MeCN (20 mL) was added Cs₂CO₃ (4.91 g, 15.06 mmol, 2.0 eq) and tert-butyl 4-(bromomethyl)piperidine-1-carboxylate (2.09 g, 7.53 mmol, 1.0 eq), the reaction mixture was stirred at 60° C. under N₂ for 16 hours. The reaction mixture was filtered, and the filtrate was concentrated under reduced pressure, the residue was purified by flash column chromatography (0˜15% ethyl acetate in petroleum ether) to afford tert-butyl 4-[[5-(1-methoxycarbonyl-2-methyl-propyl)isoxazol-3-yl]oxymethyl]piperidine-1-carboxylate (1.3 g, 43%) as a yellow oil. MS (ESI) m/z: 397.0 [M+H]⁺.

Step 2: Preparation of methyl 3-methyl-2-[3-(4-piperidylmethoxy)isoxazol-5-yl]butanoate

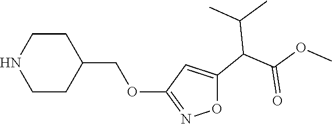 [SMILES: COC(=O)C(c1cc(OCC2CCNCC2)no1)C(C)C]

To a solution of tert-butyl 4-[[5-(1-methoxycarbonyl-2-methyl-propyl)isoxazol-3-yl]oxymethyl]piperidine-1-carboxylate (430 mg, 1.08 mmol, 1 eq) in CH₂Cl₂ (6 mL) was added HCl/dioxane (4 M, 5.73 mL). The mixture was stirred at 15° C. for 1 hour, then concentrated under reduced pressure. The residue was basified with sat. NaHCO₃ to pH 8, and extracted with dichloromethane (50 mL×3). The combined organic phase was washed with brine (20 mL), dried over anhydrous sodium sulfate, filtered, and concentrated in vacuum to give methyl 3-methyl-2-[3-(4-piperidylmethoxy)isoxazol-5-yl]butanoate (300 mg, 90% yield) as a colorless oil. MS (ESI) m/z: 297.3 [M+1]⁺.

Step 3: Preparation of methyl2-[3-[[1-[[(3S,8S)-8-[[tert-butyl(diphenyl)silyl]oxymethyl]-1,2,3,5,6,7-hexahydropyrrolizin-3-yl]methyl]-4-piperidyl]methoxy]isoxazol-5-yl]-3-meth yl-butanoate

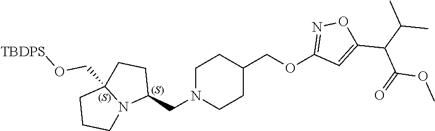

To a solution of (3S,8S)-8-[[tert-butyl(diphenyl)silyl]oxymethyl]-1,2,3,5,6,7-hexahydropyrrolizine-3-carbaldehyde (495 mg, 1.21 mmol, 1.2 eq) and methyl 3-methyl-2-[3-(4-piperidylmethoxy)isoxazol-5-yl]butanoate (300 mg, 1.01 mmol, 1 eq) in CH₂Cl₂ (10 mL) was added NaBH(OAc)₃ (472 mg, 2.23 mmol, 2.2 eq). The mixture was stirred at 15° C. for 15 hours. The reaction was quenched with sat. NaHCO₃ (20 mL), then extracted with dichloromethane (50 mL×3). The combined organic phase was washed with brine (20 mL), dried over anhydrous sodium sulfate, filtered, and concentrated in vacuum. The residue was purified by flash column chromatography (0˜3 then up to 5% methanol in dichloromethane) to afford methyl2-[3-[[1-[[(3S,8S)-8-[[tert-butyl(diphenyl)silyl]oxymethyl]-1,2,3,5,6,7-hexahydropyrrolizin-3-yl]methyl]-4-piperidyl]methoxy]isoxazol-5-yl]-3-methyl-butanoate (550 mg, 70%) as a yellow solid. MS (ESI) m/z: 688.5 [M+1]⁺.

Step 4: Preparation of methyl2-[3-[[1-[[(3S,8S)-8-(hydroxymethyl)-1,2,3,5,6,7-hexahydro pyrrolizin-3-yl]methyl]-4-piperidyl]methoxy]isoxazol-5-yl]-3-methyl-butanoate

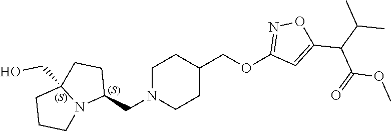

To a solution of methyl 2-[3-[[1-[[(3S,8S)-8-[[tert-butyl(diphenyl)silyl]oxymethyl]-1,2,3,5,6,7-hexahydropyrrolizin-3-yl]methyl]-4-piperidyl]methoxy]isoxazol-5-yl]-3-methyl-butanoate (518 mg, 0.75 mmol, 1 eq) in CH₂Cl₂ (12 mL) was added N,N-diethylethanamine; trihydrofluoride (1.82 g, 11.29 mmol, 15 eq). The mixture was stirred at 15° C. for 2 hours, then concentrated under reduced pressure. The residue was diluted with water (10 mL), and extracted with MTBE (20 mL×3). The aqueous phase was basified with sat. NaHCO₃ to pH 8 and then extracted with dichloromethane (40 mL×3). The combined organic phase was washed with brine (20 mL), dried over anhydrous sodium sulfate, filtered, and concentrated in vacuum to give methyl2-[3-[[1-[[(3S,8S)-8-(hydroxymethyl)-1,2,3,5,6,7-hexahydropyrrolizin-3-yl]methyl]-4-piperidyl]methoxy]isoxazol-5-yl]-3-methyl-butanoate (320 mg, crude) as a yellow solid. MS (ESI) m/z: 450.2 [M+1]⁺.

Step 5: Preparation of tert-butyl 3-[7-[8-ethyl-3-(methoxymethoxy)-1-naphthyl]-8-fluoro-2-[[(3S,8S)-3-[[4-[[5-(1-methoxycarbonyl-2-methyl-propyl)isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

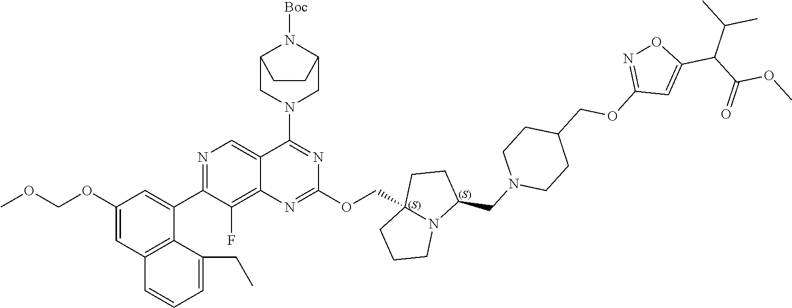

To a solution of tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (450 mg, 0.69 mmol, 1 eq), 4 A MS (450 mg, 1.0 eq) and methyl 2-[3-[[1-[[(3S,8S)-8-(hydroxymethyl)-1,2,3,5,6,7-hexahydropyrrolizin-3-yl]methyl]-4-piperidyl]methoxy]isoxazol-5-yl]-3-methyl-butanoate (310 mg, 0.69 mmol, 1 eq) in THF (5 mL) was added t-BuONa (166 mg, 1.73 mmol, 2.5 eq) at 0° C. under N₂. The mixture was stirred at 0° C. for 20 minutes, then added to aq. HCl (0.2 M) to pH 8, extracted with CH₂Cl₂ (20 mL×3). The combined organic phase was washed with brine (20 mL), dried over anhydrous sodium sulfate, filtered, and concentrated in vacuum. The residue was purified by flash column chromatography (0˜5% then up to 8% methanol in dichloromethane) to afford tert-butyl 3-[7-[8-ethyl-3-(methoxymethoxy)-1-naphthyl]-8-fluoro-2-[[(3S,8S)-3-[[4-[[5-(1-methoxycarbonyl-2-methyl-propyl)isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (460 mg, 55%) as a yellow solid. MS (ESI) m/z: 1021.5 [M+1]⁺.

Step 6: Preparation of 2-[3-[[1-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]-4-piperidyl]methoxy]isoxazol-5-yl]-3-methyl-butanoic acid

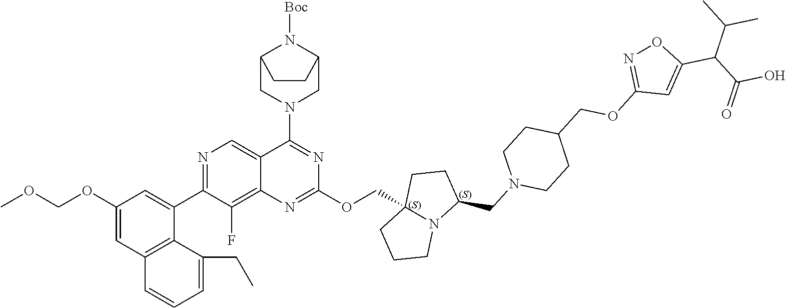

To a solution of tert-butyl 3-[7-[8-ethyl-3-(methoxymethoxy)-1-naphthyl]-8-fluoro-2-[[(3S,8S)-3-[[4-[[5-(1-methoxycarbonyl-2-methyl-propyl)isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (460 mg, 0.45 mmol, 1 eq) in THF (5 mL) was added a solution of LiOH·H₂O (57 mg, 1.35 mmol, 3.0 eq) in H₂O (2 mL). The mixture was stirred at 16° C. for 15 hours, then concentrated under reduced pressure. The residue was acidified with aqueous HCl (2 N) to pH 4, extracted with dichloromethane/MeOH (80 mL×3, 10/1). The combined organic phase was washed with brine (20 mL), dried over anhydrous sodium sulfate, filtered, and concentrated in vacuum to give 2-[3-[[1-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]-4-piperidyl]methoxy]isoxazol-5-yl]-3-methyl-butanoic acid (450 mg, crude) as a yellow solid, which was used in the next step without further purification. MS (ESI) m/z: 1007.5 [M+1]⁺.

Step 7: Preparation of tert-butyl 3-[7-[8-ethyl-3-(methoxymethoxy)-1-naphthyl]-8-fluoro-2-[[(3S,8S)-3-[[4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate and tert-butyl 3-[7-[8-ethyl-3-(methoxymethoxy)-1-naphthyl]-8-fluoro-2-[[(3S,8S)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

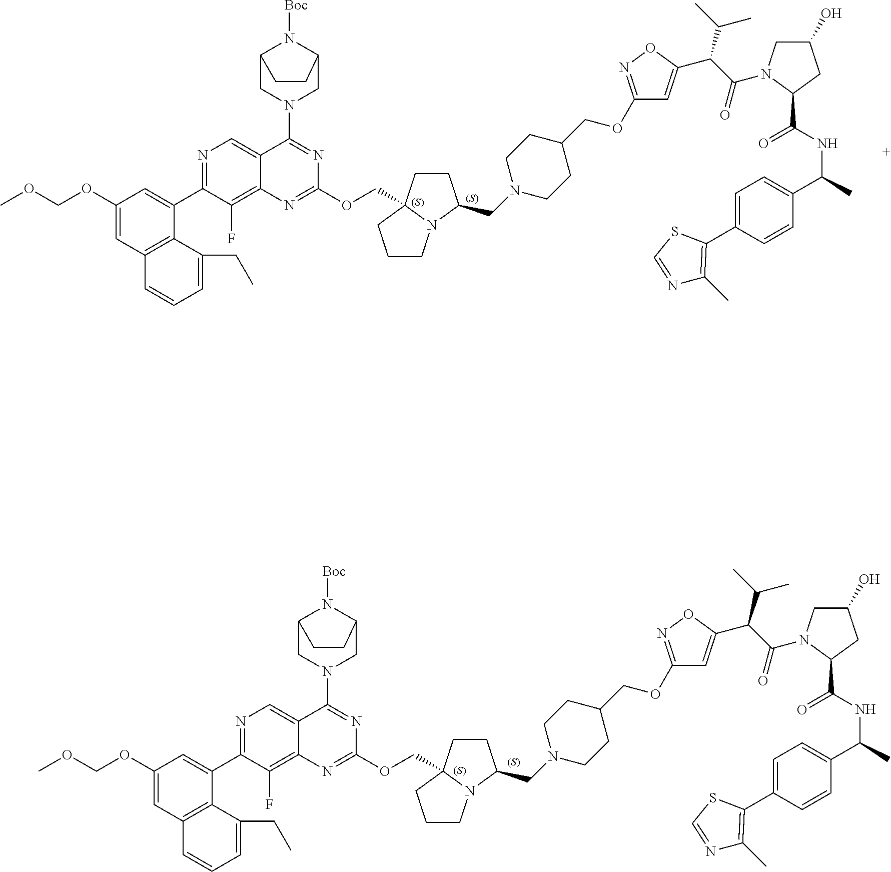

To a solution of 2-[3-[[1-[[(3S,8S)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl]-4-piperidyl]methoxy]isoxazol-5-yl]-3-methyl-butanoic acid (450 mg, 0.45 mmol, 1 eq) and (2S,4R)-4-hydroxy-N-[(1S)-1-[4-(4-methylthiazol-5-yl)phenyl]ethyl]pyrrolidine-2-carboxamide (208 mg, 0.49 mmol, 1.1 eq, HCl salt) in DMF (7.0 mL) was added DIEA (289 mg, 2.23 mmol, 5 eq) and HATU (187 mg, 0.49 mmol, 1.1 eq). The mixture was stirred at 15° C. for 16 hours, then added to water (42 mL), stirred for a while and filtered, the filter cake was washed with water (10 mL), diluted with CH₂Cl₂ (50 mL), dried over anhydrous sodium sulfate, filtered, and concentrated in vacuum. The residue was purified by flash column chromatography (0˜5% then up to 10% methanol in dichloromethane) to afford tert-butyl3-[7-[8-ethyl-3-(methoxymethoxy)-1-naphthyl]-8-fluoro-2-[[(3S,8S)-3-[[4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (340 mg, 51%) as a yellow solid. MS (ESI) m/z: 661.1 [M/2+1]⁺. The material was further separated by SFC (mobile phase: [ACN/EtOH (0.1% NH₃H₂O)]; B %: 30%-30%) to afford tert-butyl 3-[7-[8-ethyl-3-(methoxymethoxy)-1-naphthyl]-8-fluoro-2-[[(3S,8S)-3-[[4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (120 mg, 27%) as a yellow solid, and tert-butyl3-[7-[8-ethyl-3-(methoxymethoxy)-1-naphthyl]-8-fluoro-2-[[(3S,8S)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (140 mg, 34%) as a yellow solid. MS (ESI) m/z: 1321.7 [M+1]⁺.

Step 8: Preparation of (2S,4R)-1-[(2R)-2-{3-[(1-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

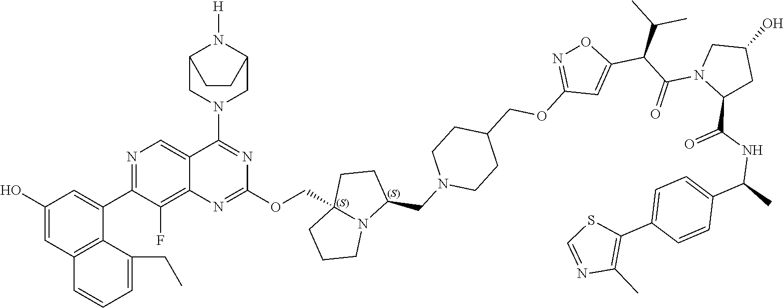

To a solution of tert-butyl 3-[7-[8-ethyl-3-(methoxymethoxy)-1-naphthyl]-8-fluoro-2-[[(3S,8S)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (130 mg, 0.1 mmol, 1 eq) in CH₂Cl₂ (3 mL) was added HCl/dioxane (4 M, 3 mL) was stirred at 20° C. for 2 hours. The mixture was suspended in petroleum ether (20 mL), filtered. The filter cake was diluted with CH₂Cl₂ (10 mL) and Et₃N (3 mL), then concentrated in vacuum. The residue was diluted with DMF (4 mL), filtered and purified by prep-HPLC (column: Phenomenex C18 75*30 mm*3 um; mobile phase: [water (FA)-ACN]; B %: 10%-50%, 28 min) to afford (2S,4R)-1-[(2R)-2-{3-[(1-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (60.9 mg, 52%) as a yellow solid. MS (ESI) m/z: 1177.0 [M+1]⁺; ¹H NMR (400 MHz, CD₃OD) δ 9.13 (s, 1H), 8.88 (s, 1H), 8.42 (s, 2H), 7.64 (d, J=8.0 Hz, 1H), 7.48-7.34 (m, 5H), 7.31-7.27 (m, 1H), 7.20-7.12 (m, 1H), 7.01 (s, 1H), 6.00-5.88 (m, 1H), 5.08-4.97 (m, 1H), 4.86-4.67 (m, 3H), 4.64-4.56 (m, 1H), 4.54-4.47 (m, 1H), 4.45-4.37 (m, 1H), 4.26-4.12 (m, 1H), 4.09-3.79 (m, 7H), 3.75-3.49 (m, 3H), 3.37-3.33 (m, 1H), 3.26-3.10 (m, 1H), 3.01-2.91 (m, 1H), 2.87-2.74 (m, 1H), 2.68-2.55 (m, 1H), 2.52-2.45 (m, 3H), 2.44-1.91 (m, 19H), 1.88-1.71 (m, 3H), 1.62-1.26 (m, 5H), 1.10-1.01 (m, 3H), 0.96-0.82 (m, 6H).

Exemplary Synthesis of (2S,4R)-1-[(2S)-2-{3-[(1-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 97)

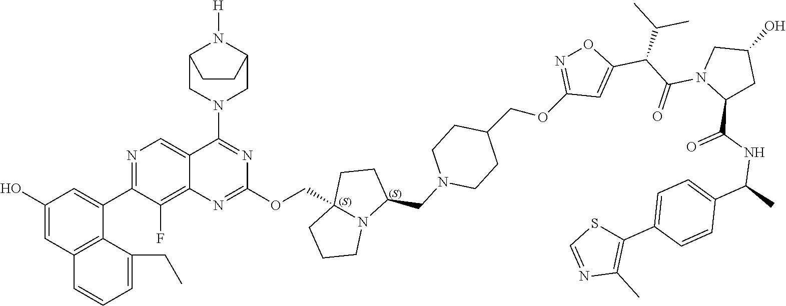

The title compound was prepared in an analogous manner to Compound 96 starting from tert-butyl 3-[7-[8-ethyl-3-(methoxymethoxy)-1-naphthyl]-8-fluoro-2-[[(3S,8S)-3-[[4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate, and purified by prep-HPLC (column: Phenomenex C18 75*30 mm*3 um; mobile phase: [water (FA)-ACN]; B %: 8%-48%, 28 min) to afford (2S,4R)-1-[(2S)-2-{3-[(1-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (45.8 mg, 41% yield, FA salt) as a yellow solid. MS (ESI) m/z: 1176.4 [M+1]⁺; 1H NMR (400 MHz, CD₃OD) δ 9.11 (s, 1H), 8.93-8.83 (m, 1H), 8.43 (br s, 2H), 7.63 (d, J=8.0 Hz, 1H), 7.49-7.26 (m, 6H), 7.16 (d, J=7.2 Hz, 1H), 7.04-6.95 (m, 1H), 6.02-5.91 (m, 1H), 5.03-4.94 (m, 1H), 4.81-4.66 (m, 3H), 4.64-4.52 (m, 2H), 4.46-4.34 (m, 1H), 4.27-4.11 (m, 1H), 4.08-3.79 (m, 6H), 3.78-3.55 (m, 4H), 3.40-3.32 (m, 1H), 3.23-3.12 (m, 1H), 3.0-2.87 (m, 1H), 2.85-2.75 (m, 1H), 2.66-2.56 (m, 1H), 2.45 (s, 3H), 2.41-1.90 (m, 19H), 1.84-1.68 (m, 3H), 1.60-1.21 (m, 5H), 1.05 (d, J=6.8 Hz, 3H), 0.97-0.79 (m, 6H).

Exemplary Synthesis of (2S,4R)-1-[(2R*)-2-[3-(7-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-{[4-(4-methyl-1,3-thiazol-5-yl)phenyl]methyl}pyrrolidine-2-carboxamide (Compound 98)

Step 1: Preparation of tert-butyl 2-[5-[1-[(2R,4S)-4-hydroxy-2-[[4-(4-methylthiazol-5-yl)phenyl]methylcarbamoyl]pyrrolidine-1-carbonyl]-2-methyl-propyl]isoxazol-3-yl]-2,7-di azaspiro[3.5]nonane-7-carboxylate

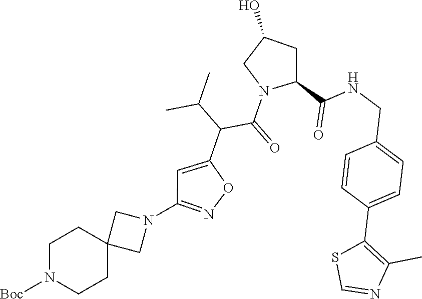 [SMILES: Cc1ncsc1-c1ccc(CNC(=O)[C@@H]2C[C@@H](O)CN2C(=O)C(c2cc(N3CC4(CCN(C(=O)OC(C)(C)C)CC4)C3)no2)C(C)C)cc1]

To a mixture of (2R,4S)-4-hydroxy-N-[[4-(4-methylthiazol-5-yl)phenyl]methyl]pyrrolidine-2-carboxamide (2.16 g, 6.10 mmol, 1 eq, hydrochloride) and 2-[3-(7-tert-butoxycarbonyl-2,7-diazaspiro[3.5]nonan-2-yl)isoxazol-5-yl]-3-methyl-butanoic acid (2.4 g, 6.10 mmol, 1 eq) in N,N-dimethylformamide (30 mL) was added O-(7-Azabenzotriazol-1-yl)-N,N,N′,N′-tetramethyluronium hexafluorophosphate (3.48 g, 9.15 mmol, 1.5 eq) and triethylamine (3.09 g, 30.50 mmol, 4.2 mL, 5 eq) in one portion at 20° C. under nitrogen. The mixture was stirred at 20° C. for 2 h, then poured into ice-water (w/w=1/1, 50 mL) and stirred for 5 min. The aqueous phase was extracted with ethyl acetate (50 mL×4). The combined organic phase was washed with brine (20 mL×2), dried over anhydrous sodium sulfate, filtered, and concentrated in vacuum. The residue was purified by prep-HPLC (mobile phase: [water (FA)-ACN]; B %: 45%-70%, 20 min) to afford tert-butyl 2-[5-[1-[(2R,4S)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonane-7-carboxylate (2.8 g, 66%) as a yellow solid.

Step 2: Preparation of tert-butyl 2-[5-[(1S)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonane-7-carboxylate and tert-butyl 2-[5-[(1R)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonane-7-carboxylate

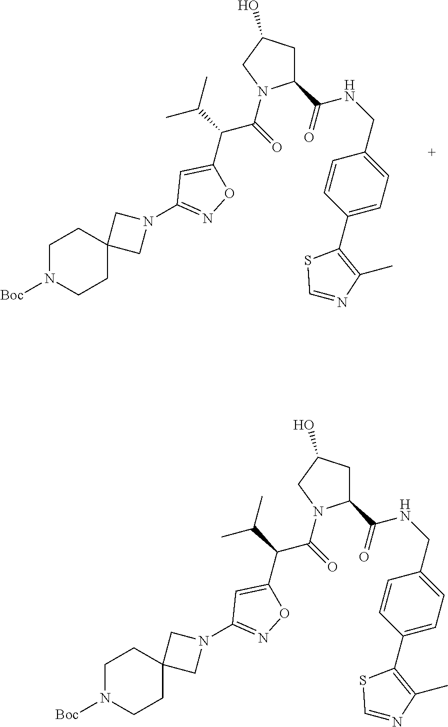 [SMILES: Cc1ncsc1-c1ccc(CNC(=O)[C@@H]2C[C@@H](O)CN2C(=O)[C@H](c2cc(N3CC4(CCN(C(=O)OC(C)(C)C)CC4)C3)no2)C(C)C)cc1 ; Cc1ncsc1-c1ccc(CNC(=O)[C@@H]2C[C@@H](O)CN2C(=O)[C@@H](c2cc(N3CC4(CCN(C(=O)OC(C)(C)C)CC4)C3)no2)C(C)C)cc1]

Tert-butyl 2-[5-[1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonane-7-carboxylate (1.96 g, 2.83 mmol) was separated by SFC (column: REGIS (S,S) WHELK-O1 250×25 mm, I.D., 10 um; mobile phase: acetonitrile/isopropanol (0.1% NH₃H₂O) in CO₂ from 40% to 40%; flow rate: 70 mL/min; 220 nm) to afford tert-butyl 2-[5-[(1S)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonane-7-carboxylate (602 mg, 31%) and tert-butyl 2-[5-[(1R)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonane-7-carboxylate (1.24 g, 63%) as a yellow solid.

Step 3: Preparation of (2S,4R)-1-[(2R)-2-[3-(2,7-diazaspiro[3.5]nonan-2-yl)isoxazol-5-yl]-3-methyl-butanoyl]-4-hydroxy-N-[[4-(4-methylthiazol-5-yl)phenyl]methyl]pyrrolidine-2-carboxamide

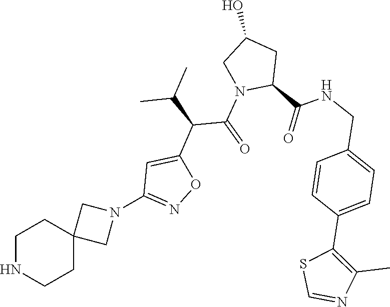 [SMILES: Cc1ncsc1-c1ccc(CNC(=O)[C@@H]2C[C@@H](O)CN2C(=O)[C@@H](c2cc(N3CC4(CCNCC4)C3)no2)C(C)C)cc1]

To a solution of tert-butyl 2-[5-[(1R)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonane-7-carboxylate (100 mg, 0.14 mmol, 1 eq) in dichloromethane (3 mL) was added trifluoroacetic acid (924 mg, 8.10 mmol, 0.6 mL, 71.61 eq). The reaction solution was stirred at 20° C. for 1 h, then concentrated under vacuum to afford (2S,4R)-1-[(2R)-2-[3-(2,7-diazaspiro[3.5]nonan-2-yl)isoxazol-5-yl]-3-methyl-butanoyl]-4-hydroxy-N-[[4-(4-methylthiazol-5-yl)phenyl]methyl]pyrrolidine-2-carboxamide (103 mg, crude, trifluoroacetate) as yellow oil.

Step 4: Preparation of tert-butyl 3-[7-(8-ethyl-3-hydroxy-1-naphthyl)-8-fluoro-2-[2-[2-[5-[(1R)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate

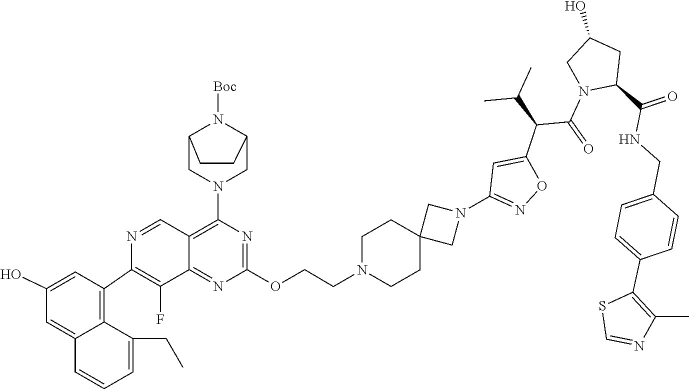 [SMILES: CCc1cccc2cc(O)cc(-c3ncc4c(N5CC6CCC(C5)N6C(=O)OC(C)(C)C)nc(OCCN5CCC6(CC5)CN(c5cc([C@H](C(=O)N7C[C@H](O)C[C@H]7C(=O)NCc7ccc(-c8scnc8C)cc7)C(C)C)on5)C6)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (90 mg, 0.15 mmol, 1 eq) and (2S,4R)-1-[(2R)-2-[3-(2,7-diazaspiro[3.5]nonan-2-yl)isoxazol-5-yl]-3-methyl-butanoyl]-4-hydroxy-N-[[4-(4-methylthiazol-5-yl)phenyl]methyl]pyrrolidine-2-carboxamide (103 mg, 0.15 mmol, 0.95 eq, trifluoroacetate) in N,N-dimethylformamide (3 mL) was added 4-methylmorpholine (34 mg, 0.34 mmol, 3 eq) and sodium triacetoxyborohydride (97 mg, 0.46 mmol, 3 eq). The reaction was stirred at 25° C. for 12 h, then diluted with a mixture of ethyl acetate/tetrahydrofuran (v/v=10/1, 200 mL), washed with water (20 mL×3). The organic layer was dried over anhydrous sodium sulfate, filtered and concentrated under vacuum. The residue was purified by prep-TLC (dichloromethane/methanol=10/1) to afford tert-butyl 3-[7-(8-ethyl-3-hydroxy-1-naphthyl)-8-fluoro-2-[2-[2-[5-[(1R)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (41 mg, 23%) as a yellow solid. MS (ESI) m/z: 583.0 [M/2+1]⁺.

Step 5: Preparation of (2S,4R)-1-[(2R*)-2-[3-(7-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-{[4-(4-methyl-1,3-thiazol-5-yl)phenyl]methyl}pyrrolidine-2-carboxamide

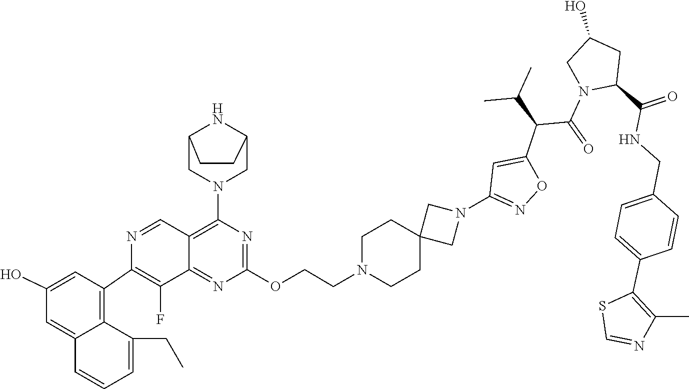 [SMILES: CCc1cccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)NCc7ccc(-c8scnc8C)cc7)C(C)C)on5)C6)nc4c3F)c12]

To a solution of tert-butyl 3-[7-(8-ethyl-3-hydroxy-1-naphthyl)-8-fluoro-2-[2-[2-[5-[(1S)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonan-7-yl]ethoxy]pyrido[4,3-d]pyrimidin-4-yl]-3,8-diazabicyclo[3.2.1]octane-8-carboxylate (41 mg, 0.04 mmol, 1 eq) in dichloromethane (3 mL) was added trifluoroacetic acid (462 mg, 4.05 mmol, 0.3 mL, 40.06 eq). The reaction solution was stirred at 20° C. for 0.5 h, then concentrated under vacuum. The residue was purified by prep-HPLC (column: Phenomenex C18 75*30 mm*3 um; mobile phase: [water (FA)-ACN];

B %: 10%-40%, 7 min) to afford (2S,4R)-1-[(2R*)-2-[3-(7-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-{[4-(4-methyl-1,3-thiazol-5-yl)phenyl]methyl}pyrrolidine-2-carboxamide (13.6 mg, 33%) as a white solid. MS (ESI) m/z: 1064.4 [M+1]⁺; 1H NMR (400 MHz, DMSO-d₆) δ 9.13 (s, 1H), 9.02-8.95 (m, 1H), 8.43 (t, J=6.0 Hz, 1H), 8.17 (s, 1H), 7.67 (d, J=8.0 Hz, 1H), 7.49-7.31 (m, 5H), 7.29 (d, J=2.8 Hz, 1H), 7.13 (br d, J=7.2 Hz, 1H), 6.97 (d, J=2.8 Hz, 1H), 5.84-5.78 (m, 1H), 4.64-4.19 (m, 9H), 3.90 (br d, J=11.2 Hz, 2H), 3.78 (br d, J=12.8 Hz, 1H), 3.70 (br d, J=8.8 Hz, 2H), 3.62-3.53 (m, 3H), 3.51-3.40 (m, 4H), 2.76-2.63 (m, 2H), 2.47-2.33 (m, 7H), 2.30-2.18 (m, 3H), 2.10-2.02 (m, 1H), 1.95-1.88 (m, 1H), 1.81 (br s, 4H), 1.73 (br s, 1H), 1.63 (br s, 3H), 0.96 (d, J=6.8 Hz, 2H), 0.89-0.79 (m, 5H), 0.67 (d, J=6.8 Hz, 1H), 0.59 (d, J=6.8 Hz, 1H).

Exemplary Synthesis of (2S,4R)-1-[(2R*)-2-[3-(7-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-{[4-(4-methyl-1,3-thiazol-5-yl)phenyl]methyl}pyrrolidine-2-carboxamide (Compound 99)

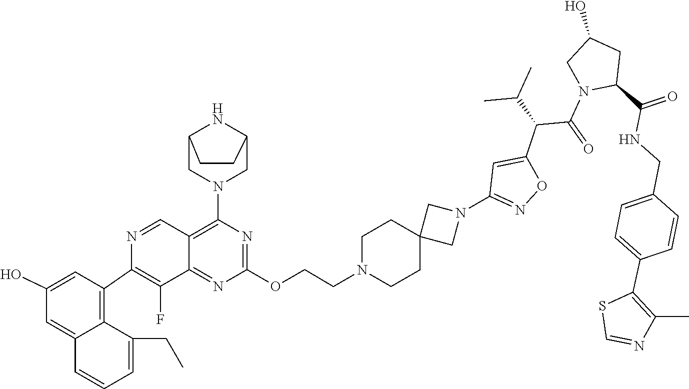 [SMILES: CCc1cccc2cc(O)cc(-c3ncc4c(N5CC6CCC(C5)N6)nc(OCCN5CCC6(CC5)CN(c5cc([C@@H](C(=O)N7C[C@H](O)C[C@H]7C(=O)NCc7ccc(-c8scnc8C)cc7)C(C)C)on5)C6)nc4c3F)c12]

The title compound was prepared in an analogous manner to Compound 79 starting from tert-butyl 2-[5-[(1S)-1-[(2S,4R)-4-hydroxy-2-[[4-(4-methylthiazol-5-yl)phenyl]methylcarbamoyl]pyrrolidine-1-carbonyl]-2-methyl-propyl]isoxazol-3-yl]-2,7-diazaspiro[3.5]nonane-7-carboxylate, and purified by prep-HPLC (column: Phenomenex C18 75*30 mm*3 um; mobile phase: [water (FA)-ACN]; B %: 10%-40%, 7 min) to afford (2S,4R)-1-[(2R*)-2-[3-(7-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-{[4-(4-methyl-1,3-thiazol-5-yl)phenyl]methyl}pyrrolidine-2-carboxamide (55.8 mg, 91%) as a white solid. MS (ESI) m/z: 1064.4 [M+1]⁺; 1H NMR (400 MHz, DMSO-d₆) δ 9.13 (s, 1H), 9.02-8.95 (m, 1H), 8.43 (t, J=6.0 Hz, 1H), 8.17 (s, 1H), 7.67 (d, J=8.0 Hz, 1H), 7.49-7.31 (m, 5H), 7.29 (d, J=2.8 Hz, 1H), 7.13 (br d, J=7.2 Hz, 1H), 6.97 (d, J=2.8 Hz, 1H), 5.84-5.78 (m, 1H), 4.64-4.19 (m, 9H), 3.90 (br d, J=11.2 Hz, 2H), 3.78 (br d, J=12.8 Hz, 1H), 3.70 (br d, J=8.8 Hz, 2H), 3.62-3.53 (m, 3H), 3.51-3.40 (m, 4H), 2.76-2.63 (m, 2H), 2.47-2.33 (m, 7H), 2.30-2.18 (m, 3H), 2.10-2.02 (m, 1H), 1.95-1.88 (m, 1H), 1.81 (br s, 4H), 1.73 (br s, 1H), 1.63 (br s, 3H), 0.96 (d, J=6.8 Hz, 2H), 0.89-0.79 (m, 5H), 0.67 (d, J=6.8 Hz, 1H), 0.59 (d, J=6.8 Hz, 1H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate (Compound 100)

Step 1: Preparation of 2-[3-[(tert-butoxycarbonylamino)methyl]isoxazol-5-yl]-3-methyl-butanoic acid

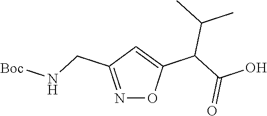 [SMILES: CC(C)C(C(=O)O)c1cc(CNC(=O)OC(C)(C)C)no1]

To a stirred solution of methyl 2-[3-[(tert-butoxycarbonylamino)methyl]isoxazol-5-yl]-3-methyl-butanoate (771 mg, 2.47 mmol, 1 eq) in MeOH (4 mL)/THF (4 mL) was added a solution of LiOH·H₂O (259 mg, 6.17 mmol, 2.5 eq) in water (4 mL), and the reaction mixture was stirred at 20° C. for 3 hours. Then adjusted the pH to 5 with 2 M aq. HCl and extracted with dichloromethane (10 mL×3). The combined organic layer was washed with brine (10 mL), dried over anhydrous Na₂SO₄, filtered, and concentrated under reduced pressure to give 2-[3-[(tert-butoxycarbonylamino)methyl]isoxazol-5-yl]-3-methyl-butanoic acid (731 mg, crude) as a yellow oil, which would be used in the next step directly. MS (ESI) m/z: 243.0 [M+H−54]⁺.

Step 2: Preparation of tert-butyl N-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]carbamate

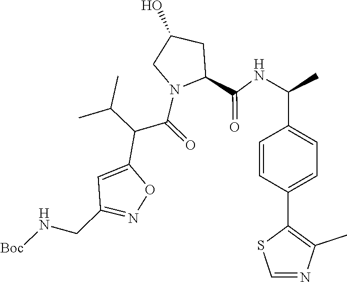 [SMILES: Cc1ncsc1-c1ccc([C@H](C)NC(=O)[C@@H]2C[C@@H](O)CN2C(=O)C(c2cc(CNC(=O)OC(C)(C)C)no2)C(C)C)cc1]

To a stirred solution of 2-[3-[(tert-butoxycarbonylamino)methyl]isoxazol-5-yl]-3-methyl-butanoic acid (731 mg, 2.45 mmol, 1 eq) in CH₂Cl₂ (20 mL) were added DIEA (1.58 g, 12.3 mmol, 2.13 mL, 5 eq) and HATU (1.21 g, 3.19 mmol, 1.3 eq), the reaction mixture was stirred at 20° C. for 10 min. Then (2S,4R)-4-hydroxy-N-[(1S)-1-[4-(4-methylthiazol-5-yl)phenyl]ethyl]pyrrolidine-2-carboxamide (1.48 g, 3.43 mmol, 1.4 eq, HCl) was added, and the reaction mixture was stirred at 20° C. for 2 hours. The reaction mixture was poured into water (20 mL) and the layers were separated. The aqueous layer was extracted with DCM (20 mL). The combined organic layer was washed with brine (20 mL), dried over anhydrous Na₂SO₄, filtered, and concentrated under reduced pressure. The residue was purified by flash column chromatography (0˜100% ethyl acetate in petroleum ether) to give tert-butyl N-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]carbamate (1.19 g, 79%) as a yellow solid. MS (ESI) m/z: 612.2 [M+H]⁺.

Step 3: Preparation of tert-butyl N-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]carbamate & tert-butyl N-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]carbamate

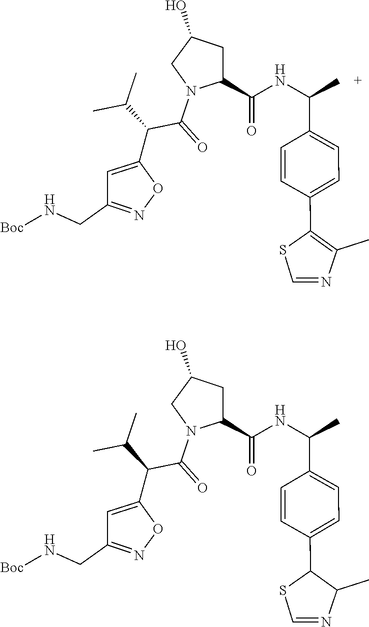

Tert-butyl N-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]carbamate (948 mg, 1.55 mmol) was separated by SFC (column: DAICEL CHIRALPAK AD (250 mm*50 mm, 10 um); mobile phase: [0.1% NH₃H₂O IPA]; B %: 50%-50%) to give tert-butyl N-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]carbamate (400 mg, 42%) as a yellow solid, and tert-butyl N-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]carbamate (457 mg, 48%) as a yellow solid. MS (ESI) m/z: 612.5 [M+H]⁺.

Step 4: Preparation of (2S,4R)-1-[(2S)-2-[3-(aminomethyl)isoxazol-5-yl]-3-methyl-butanoyl]-4-hydroxy-N-[(1S)-1-[4-(4-methylthiazol-5-yl)phenyl]ethyl]pyrrolidine-2-carboxamide

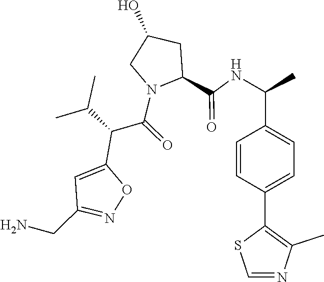 [SMILES: Cc1ncsc1-c1ccc([C@H](C)NC(=O)[C@@H]2C[C@@H](O)CN2C(=O)[C@H](c2cc(CN)no2)C(C)C)cc1]

A mixture of tert-butyl N-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methyl]carbamate (400 mg, 0.65 mmol, 1 eq) in EtOAc (2 mL) and 4 M HCl/dioxane (2 mL) was stirred at 20° C. for 1 hour. The reaction mixture was concentrated at 35° C. under reduced pressure to give (2S,4R)-1-[(2S)-2-[3-(aminomethyl)isoxazol-5-yl]-3-methyl-butanoyl]-4-hydroxy-N-[(1S)-1-[4-(4-methylthiazol-5-yl)phenyl]ethyl]pyrrolidine-2-carboxamide (426 mg, crude, HCl) as a yellow solid. MS (ESI) m/z: 512.2 [M+H]⁺.

Step 5: Preparation of tert-butyl 3-[7-[8-ethyl-3-(methoxymethoxy)-1-naphthyl]-8-fluoro-2-[[(3S,8S)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methylcarbamoyloxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

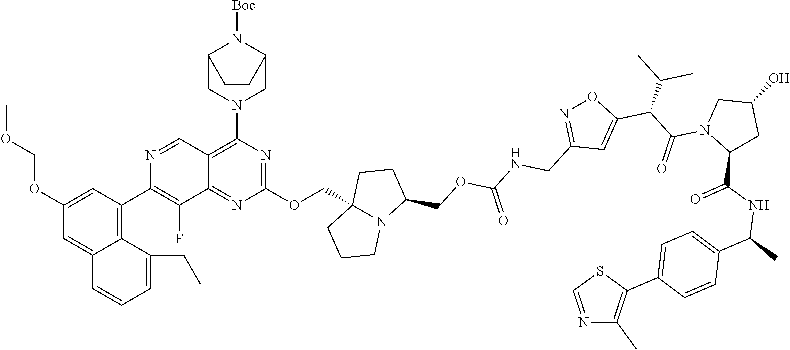 [SMILES: CCc1cccc2cc(OCOC)cc(-c3ncc4c(N5CC6CCC(C5)N6C(=O)OC(C)(C)C)nc(OC[C@@]56CCCN5[C@H](COC(=O)NCc5cc([C@@H](C(=O)N7C[C@H](O)C[C@H]7C(=O)N[C@@H](C)c7ccc(-c8scnc8C)cc7)C(C)C)on5)CC6)nc4c3F)c12]

To a solution of tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (100 mg, 134.61 umol, 1.0 eq) and (4-nitrophenyl) carbonochloridate (46 mg, 228.84 umol, 1.7 eq) in THF (3 mL) were added TEA (1.35 mmol, 187 μL, 10 eq) and DMAP (2 mg, 13.46 umol, 0.1 eq). The mixture was stirred at 40° C. for 15 hours then (2S,4R)-1-[(2S)-2-[3-(aminomethyl)isoxazol-5-yl]-3-methyl-butanoyl]-4-hydroxy-N-[(1S)-1-[4-(4-methylthiazol-5-yl)phenyl]ethyl]pyrrolidine-2-carboxamide (125 mg, 228.84 umol, 1.7 eq, HCl) added to the above mixture. The mixture was stirred at 40° C. for 1 hour, then concentrated. The residue was purified by flash column chromatography (0˜9% MeOH in CH₂Cl₂) to afford tert-butyl 3-[7-[8-ethyl-3-(methoxymethoxy)-1-naphthyl]-8-fluoro-2-[[(3S,8S)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methylcarbamoyloxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (160 mg, 85% yield) as a light-yellow solid. MS (ESI) m/z: 1280.6. [M+1]⁺.

Step 6: Preparation of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate

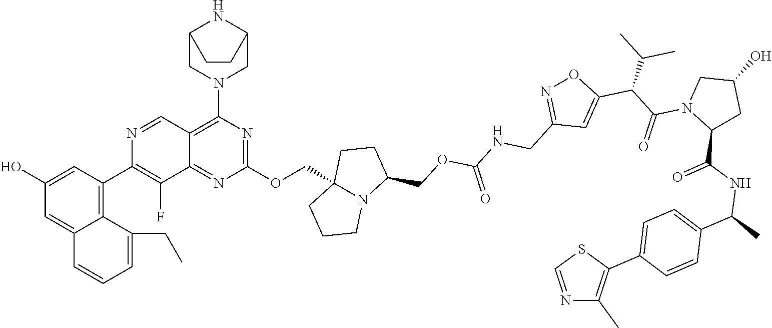 [SMILES: CCc1cccc2cc(O)cc(-c3ncc4c(N5CC6CCC(C5)N6)nc(OC[C@@]56CCCN5[C@H](COC(=O)NCc5cc([C@@H](C(=O)N7C[C@H](O)C[C@H]7C(=O)N[C@@H](C)c7ccc(-c8scnc8C)cc7)C(C)C)on5)CC6)nc4c3F)c12]

To a solution of tert-butyl 3-[7-[8-ethyl-3-(methoxymethoxy)-1-naphthyl]-8-fluoro-2-[[(3S,8S)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]methylcarbamoyloxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (160 mg, 124.95 umol, 1.0 eq) in CH₂Cl₂ (3 mL) was added HCl/EtOAc (4 M, 1.8 mL). The mixture was stirred at 15° C. for 1 hour, then concentrated. The residue was purified by prep-HPLC (15˜55% acetonitrile in water; gradient time: 28 min; hold time: 3 min; flow rate: 25 mL/min) to afford [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate (66.2 mg, 46%) as a white solid. MS (ESI) m/z: 1136.5. [M+1]⁺; 1H NMR (400 MHz, CD₃OD) δ 9.10 (s, 1H), 8.85 (s, 1H), 7.63 (d, J=8.4 Hz, 1H), 7.47-7.34 (m, 5H), 7.29 (d, J=2.4 Hz, 1H), 7.16 (d, J=7.2 Hz, 1H), 7.01-6.99 (m, 1H), 6.33-6.29 (m, 1H), 4.96-4.91 (m, 1H), 4.75 (d, J=12.0 Hz, 1H), 4.69-4.66 (m, 2H), 4.61-4.51 (m, 2H), 4.48-4.27 (m, 5H), 4.19-4.15 (m, 1H), 3.93-3.68 (m, 6H), 3.61-3.59 (m, 1H), 3.52-3.46 (m, 1H), 2.48-1.88 (m, 20H), 1.47 (d, J=7.6 Hz, 1H), 1.03 (d, J=6.0 Hz, 1H), 0.90-0.78 (m, 6H).

Exemplary Synthesis of (2S,4R)-1-[(2R)-2-[3-({1-[(2R)-2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]propyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 101)

Step 1: Preparation of methyl (2R)-2-benzyloxypropanoate

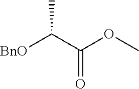

To a solution of (2R)-2-benzyloxypropanoic acid (20.0 g, 110.99 mmol, 1.0 eq) in MeOH (200 mL) was dropwise added SOCl₂ (332.96 mmol, 24.0 mL, 3.0 eq) at 0° C. The mixture was stirred at 50° C. for 4 hours, then concentrated under reduced pressure. The mixture was partitioned between sat. NaHCO₃ (80 mL) and EtOAc (100 mL). The organic phase was separated, and the aqueous phase was extracted with ethyl acetate (100 mL×3). The combined organic phase was washed with brine (100 mL), dried over Na₂SO₄, filtered and concentrated to afford the methyl (2R)-2-benzyloxypropanoate (21.56 g, crude) as a yellow oil, which was used directly in the next step without further purification. ¹H NMR (400 MHz, CDCl₃) δ 7.40-7.28 (m, 5H), 4.71 (d, J=11.6 Hz, 1H), 4.47 (d, J=11.6 Hz, 1H), 4.08 (q, J=6.8 Hz, 1H), 3.77 (s, 3H), 1.45 (d, J=6.8 Hz, 3H).

Step 2: Preparation of (2R)-2-benzyloxypropanal

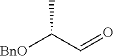

To a solution of methyl (2R)-2-benzyloxypropanoate (10 g, 51.49 mmol, 1.0 eq) in CH₂Cl₂ (100 mL) was added DIBAL-H (1 M, 56.0 mL, 1.1 eq) dropwise at −78° C. under N₂. The mixture was stirred at −78° C. for 1 hour, then quenched with water (20 mL) at −78° C. dropwise and warmed to 0° C. HCl (2 M, 20 mL) was added dropwise and warmed to 20° C. The mixture was diluted with CH₂Cl₂ (100 mL) and then filtered through celite pad under vacuum and rinsed with CH₂Cl₂ (50 mL×3). The combined organic phase was washed with brine (40 mL), dried over Na₂SO₄, filtered and concentrated in vacuum to afford the (2R)-2-benzyloxypropanal (5 g, 59%) as a yellow oil, which was used in the next step without further purification. ¹H NMR (400 MHz, CDCl₃) δ 9.68 (d, J=1.6 Hz, 1H), 7.41-7.36 (m, 4H), 4.69-4.61 (m, 2H), 3.92-3.88 (m, 1H), 1.35 (d, J=7.2 Hz, 3H).

Step 3: Preparation of [(1R)-2,2-dimethoxy-1-methyl-ethoxy]methylbenzene

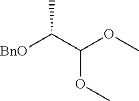

To a solution of (2R)-2-benzyloxypropanal (7.8 g, 47.50 mmol, 1.0 eq) in HC(OMe)₃ (261.27 mmol, 28.0 mL, 5.5 eq) was added PPTS (238.75 mg, 0.95 mmol, 0.02 eq). The mixture was stirred at 25° C. for 16 hours. The reaction mixture was quenched by NaHCO₃ (30 mL) at 0° C., and then extracted with ethyl acetate (80 mL×3). The combined organic phase was washed with brine (50 mL), dried over Na₂SO₄, filtered and concentrated. The residue was purified by flash column chromatography (0˜4% ethyl acetate in petroleum ether) to afford [(1R)-2,2-dimethoxy-1-methyl-ethoxy]methylbenzene (8.3 g, 83%) as a yellow oil. ¹H NMR (400 MHz, CDCl₃) δ 7.41-7.27 (m, 5H), 4.64 (s, 2H), 4.23 (d, J=5.6 Hz, 1H), 3.59-3.56 (m, 1H), 3.44 (d, J=3.6 Hz, 6H), 1.20 (d, J=6.4 Hz, 3H).

Step 4: Preparation of (2R)-1,1-dimethoxypropan-2-ol

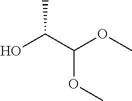

To a solution of [(1R)-2,2-dimethoxy-1-methyl-ethoxy]methylbenzene (8.3 g, 39.47 mmol, 1.0 eq) in MeOH (300 mL) was added 10% Pd/C (800 mg, 7.89 mmol, 0.2 eq) under Ar atmosphere, then the mixture was degassed and purged with H₂ for three times. The mixture was stirred at 80° C. for 10 hours under H₂ (50 psi). The mixture was filtered through celite pad, concentrated under vacuum and rinsed with ethyl acetate (100 mL×3). The filtrate was concentrated to afford (2R)-1,1-dimethoxypropan-2-ol (3.8 g, 80%) as a colorless oil, which was used in the next step without further purification. ¹H NMR (400 MHz, DMSO-d₆) δ 4.57 (s, 1H), 3.98 (d, J=5.6 Hz, 1H), 3.29 (s, 6H), 0.99 (d, J=6.2 Hz, 3H).

Step 5: Preparation of tert-butyl 3-[2-[(1R)-2,2-dimethoxy-1-methyl-ethoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

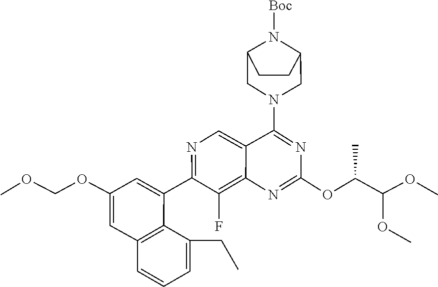

To a reaction mixture of (2R)-1,1-dimethoxypropan-2-ol (184.0 mg, 1.53 mmol, 2.0 eq) in DMF (10 mL) was added NaH (34.0 mg, 0.84 mmol, 60%, 1.1 eq) at 0° C. under N₂ atmosphere. And the mixture was stirred at 0° C. for 10 minutes. Then tert-butyl3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (500 mg, 0.77 mmol, 1.0 eq) was added to the mixture and the resulting suspension was stirred at 20° C. for 10 hours. The reaction mixture was quenched with sat. aq. NH₄Cl (20 mL) and extracted with CH₂Cl₂ (20 mL×3). The combined organic layer was washed with brine (15 mL×3), dried over Na₂SO₄, filtered and concentrated under reduced pressure. The residue was purified by flash column chromatography (0˜25% Ethyl acetate in petroleum ether) to afford tert-butyl 3-[2-[(1R)-2,2-dimethoxy-1-methyl-ethoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (300 mg, 28%) as a yellow oil. MS (ESI) m/z: 692.2 [M+H]⁺.

Step 6: Preparation of tert-butyl 3-[7-(8-ethyl-3-hydroxy-1-naphthyl)-8-fluoro-2-[(1R)-1-methyl-2-oxo-ethoxy]pyrido[4,3-d]pyrimidin-4-yl]-3,8-diazabicyclo[3.2.1]octane-8-carboxylate

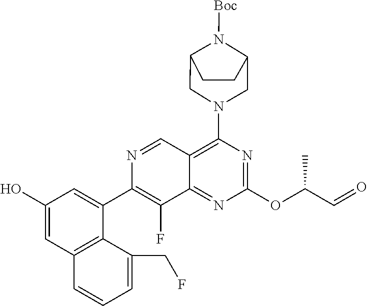 [SMILES: C[C@H](C=O)Oc1nc(N2CC3CCC(C2)N3C(=O)OC(C)(C)C)c2cnc(-c3cc(O)cc4cccc(CF)c34)c(F)c2n1]

To a stirred solution of tert-butyl 3-[2-[(1R)-2,2-dimethoxy-1-methyl-ethoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (280 mg, 404.75 umol, 1.0 eq) in acetone (607 uL) was added HCl (12 M, 607 μL, 18.0 eq), and the reaction mixture was stirred at 25° C. for 30 min. The reaction mixture was poured into a solution of NaHCO₃ (1.16 g, 13.85 mmol, 34.0 eq) in water (10 mL)/THF (3 mL). Then Boc₂O (0.45 mmol, 103.0 uL, 1.11 eq) was added, and the resulting mixture was stirred at 25° C. for 1 hour. The reaction mixture was diluted with water (50 mL) and extracted with CH₂Cl₂ (50 mL×3), the combined organic layer was dried over anhydrous Na₂SO₄, filtered, and concentrated under reduced pressure. The residue was purified by flash column chromatography (0˜43% Ethyl acetate in petroleum ether) to afford tert-butyl3-[7-(8-ethyl-3-hydroxy-1-naphthyl)-8-fluoro-2-[(1R)-1-methyl-2-oxo-ethoxy]pyrido[4,3-d]pyrimidin-4-yl]-3,8-diazabicyclo[3.2.1]octane-8-carboxylate (110 mg, 45%) as a yellow solid. MS (ESI) m/z: 602.1 [M+H]⁺.

Step 7: Preparation of tert-butyl 3-[7-(8-ethyl-3-hydroxy-1-naphthyl)-8-fluoro-2-[(1R)-2-[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]-1-methyl-ethoxy]pyrido[4,3-d]pyrimidin-4-yl]-3,8-diazabicyclo[3.2.1]octane-8-carboxylate

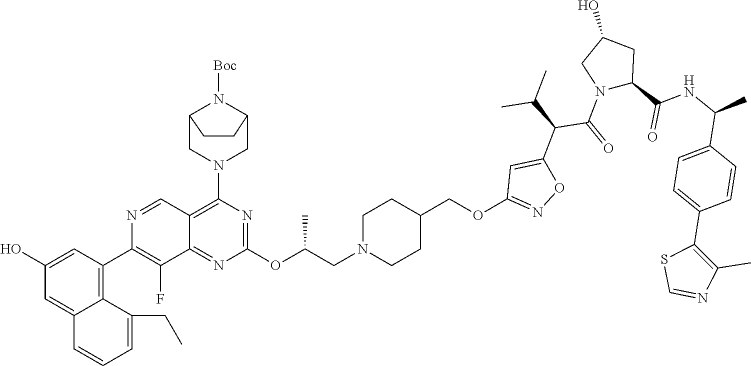 [SMILES: CCc1cccc2cc(O)cc(-c3ncc4c(N5CC6CCC(C5)N6C(=O)OC(C)(C)C)nc(O[C@H](C)CN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (109.0 mg, 0.18 mmol, 1.0 eq) and tert-butyl 3-[7-(8-ethyl-3-hydroxy-1-naphthyl)-8-fluoro-2-[(1R)-1-methyl-2-oxo-ethoxy]pyrido[4,3-d]pyrimidin-4-yl]-3,8-diazabicyclo[3.2.1]octane-8-carboxylate (110 mg, 0.18 mmol, 1.0 eq) in CH₂Cl₂ (5 mL) and i-PrOH (5 mL) were added AcOH (0.914 mmol, 52.0 uL, 5.0 eq) and borane 2-methylpyridine (98.0 mg, 0.91 mmol, 5.0 eq). The mixture was stirred at 25° C. for 4 hours. The pH of the mixture was adjusted to 8 and then concentrated to afford crude product, which was purified by flash column chromatography (0˜7% methanol in dichloromethane) to afford tert-butyl 3-[7-(8-ethyl-3-hydroxy-1-naphthyl)-8-fluoro-2-[(1R)-2-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]-1-methyl-ethoxy]pyrido[4,3-d]pyrimidin-4-yl]-3,8-diazabicyclo[3.2.1]octane-8-carboxylate (150 mg, 69%) as a yellow solid. MS (ESI) m/z: 1181.3 [M+1]⁺.

Step 8: Preparation of (2S,4R)-1-[(2R)-2-[3-({1-[(2R)-2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]propyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide

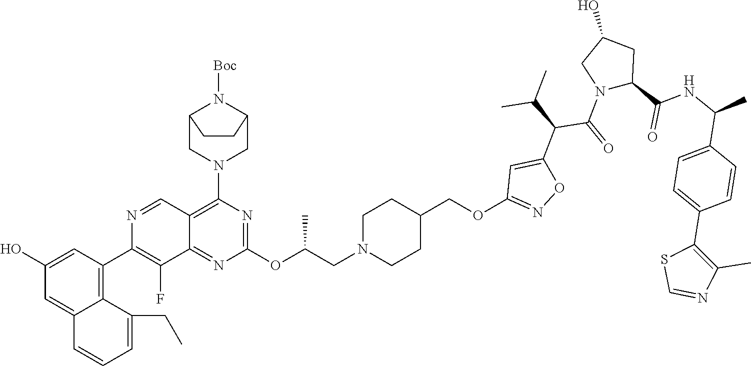 [SMILES: CCc1cccc2cc(O)cc(-c3ncc4c(N5CC6CCC(C5)N6C(=O)OC(C)(C)C)nc(O[C@H](C)CN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

A solution of tert-butyl 3-[7-(8-ethyl-3-hydroxy-1-naphthyl)-8-fluoro-2-[(1R)-2-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]-1-methyl-ethoxy]pyrido[4,3-d]pyrimidin-4-yl]-3,8-diazabicyclo[3.2.1]octane-8-carboxylate (120.0 mg, 0.10 mmol, 1.0 eq) in HCOOH (10 mL) was stirred at 25° C. for 2 hours. The mixture was concentrated to afford the crude product, which was purified by preparative HPLC (column: Phenomenex C18 75*30 mm*3 um; mobile phase: [water (FA)-ACN]; B %: 0%-40%, 28 min) to afford (2S,4R)-1-[(2R)-2-[3-({1-[(2R)-2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]propyl]piperidin-4-yl}methoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (64.5 mg, 56%, FA) as a white solid. MS (ESI) m/z: 1081.5 [M+1]⁺; ¹H NMR (400 MHz, CD₃OD) δ 9.08 (s, 1H), 8.90-8.83 (m, 1H), 8.50 (s, 1H), 7.64-76.62 m, 1H), 7.48-7.26 (m, 6H), 7.16-7.14 (m, 1H), 7.05-6.97 (m, 1H), 6.00-5.86 (m, 1H), 5.75-5.59 (m, 1H), 5.06-4.96 (m, 2H), 4.77-4.59 (m, 3H), 4.54-4.35 (m, 2H), 4.02-4.01 (m, 2H), 3.94-3.58 (m, 7H), 3.16-0.04 (m, 1H), 2.93-2.78 (m, 1H), 2.59-2.42 (m, 5H), 2.39-2.15 (m, 4H), 2.01-1.75 (m, 8H), 1.61-1.38 (m, 8H), 1.04 (d, J=6.4 Hz, 3H), 0.95-0.85 (m, 6H).

Exemplary Synthesis of [(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate (Compound 102)

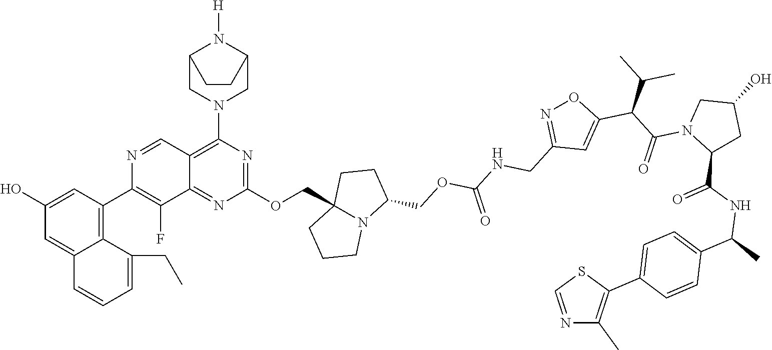 [SMILES: CCc1cccc2cc(O)cc(-c3ncc4c(N5CC6CCC(C5)N6)nc(OC[C@]56CCCN5[C@@H](COC(=O)NCc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 100 starting from (2S,4R)-1-[(2R)-2-[3-(aminomethyl)isoxazol-5-yl]-3-methyl-butanoyl]-4-hydroxy-N-[(1S)-1-[4-(4-methylthiazol-5-yl)phenyl]ethyl]pyrrolidine-2-carboxamide, and purified by prep-HPLC (column: Phenomenex C18 75*30 mm*3 um; mobile phase: [water (FA)-ACN]; B %: 10%-40%, Gradient Time: 25 min, Flow Rate: 25 ml/min) to give [(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate (68.2 mg, 55%, FA) as a white solid. MS (ESI) m/z: 1136.5 [M+H]⁺; 1H NMR (CD₃OD, 400 MHz) δ 9.18-9.09 (m, 1H), 8.91-8.86 (m, 1H), 8.49 (s, 1H), 7.65 (d, J=8.0 Hz, 1H), 7.50-7.34 (m, 5H), 7.33-7.29 (m, 1H), 7.18 (d, J=6.8 Hz, 1H), 7.06-6.99 (m, 1H), 6.39-6.27 (m, 1H), 5.04 (q, J=6.8 Hz, 1H), 4.83-4.70 (m, 3H), 4.65-4.58 (m, 1H), 4.53-4.32 (m, 6H), 4.27-4.12 (m, 1H), 4.02-3.70 (m, 6H), 3.65-3.48 (m, 2H), 2.51-1.90 (m, 21H), 1.62-1.48 (m, 3H), 1.11-1.02 (m, 3H), 0.95-0.78 (m, 6H).

Exemplary Synthesis of [(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate (Compound 103)

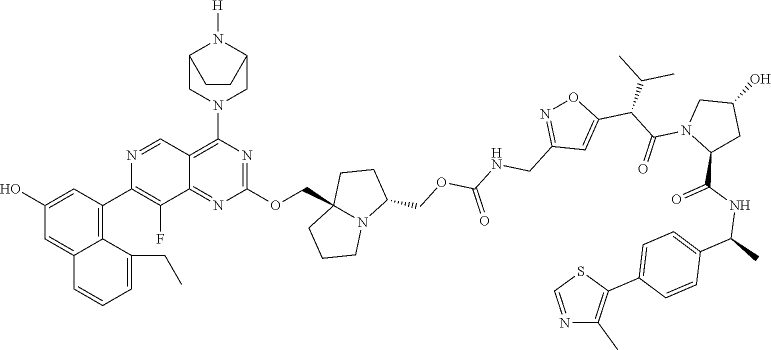 [SMILES: CCc1cccc2cc(O)cc(-c3ncc4c(N5CC6CCC(C5)N6)nc(OC[C@]56CCCN5[C@@H](COC(=O)NCc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 100 starting from (2S,4R)-1-[(2S)-2-[3-(aminomethyl)isoxazol-5-yl]-3-methyl-butanoyl]-4-hydroxy-N-[(1S)-1-[4-(4-methylthiazol-5-yl)phenyl]ethyl]pyrrolidine-2-carboxamide, and purified by prep-HPLC (column: Phenomenex C18 75*30 mm*3 um; mobile phase: [water (FA)-ACN]; B %: 10%-40%, Gradient Time: 25 min, Flow Rate: 25 ml/min) to give [(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate (55.6 mg, 47%, FA) as a white solid. MS (ESI) m/z: 1136.5 [M+H]⁺; ¹H NMR (CD₃OD, 400 MHz) δ 9.11 (s, 1H), 8.89-8.83 (m, 1H), 8.49 (s, 2H), 7.63 (d, J=8.4 Hz, 1H), 7.52-7.24 (m, 6H), 7.16 (d, J=6.8 Hz, 1H), 7.04-6.99 (m, 1H), 6.38-6.27 (m, 1H), 4.98-4.94 (m, 1H), 4.82-4.67 (m, 3H), 4.62-4.53 (m, 2H), 4.51-4.29 (m, 5H), 4.23-4.10 (m, 1H), 4.03-3.77 (m, 5H), 3.74-3.66 (m, 1H), 3.65-3.55 (m, 1H), 3.54-3.44 (m, 1H), 2.50-1.85 (m, 21H), 1.60-1.41 (m, 3H), 1.09-0.92 (m, 3H), 0.92-0.73 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate (Compound 104)

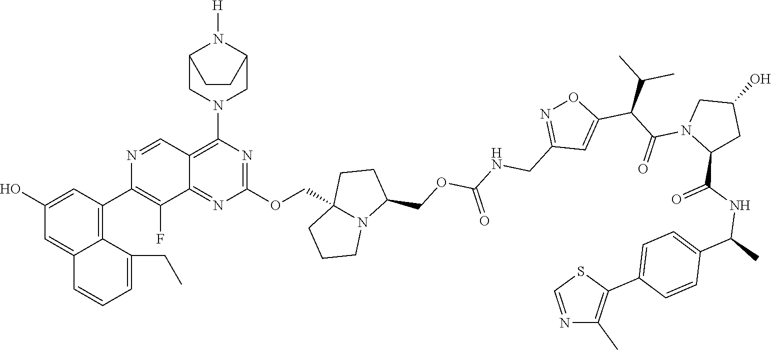 [SMILES: CCc1cccc2cc(O)cc(-c3ncc4c(N5CC6CCC(C5)N6)nc(OC[C@@]56CCCN5[C@H](COC(=O)NCc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 100 starting from (2S,4R)-1-[(2R)-2-[3-(aminomethyl)isoxazol-5-yl]-3-methyl-butanoyl]-4-hydroxy-N-[(1S)-1-[4-(4-methylthiazol-5-yl)phenyl]ethyl]pyrrolidine-2-carboxamide, and purified by prep-HPLC (Column: Phenomenex C18 75*30 mm*3 um; Eluent: gradient 8%-48% water (FA)-ACN; Gradient time: 26 min; Hold time: 4 min; Flow rate: 25 mL/min) to afford [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl N-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}methyl) carbamate (69.4 mg, 62%) as a white solid. MS (ESI) m/z: 1136.9 [M+1]⁺; 1H NMR (400 MHz, MeOD-d₄) δ 9.13-9.10 (m, 1H), 8.91-8.83 (m, 1H), 8.44 (br s, 1H), 7.66-7.61 (m, 1H), 7.44-7.35 (m, 5H), 7.30 (d, J=2.4 Hz, 1H), 7.19-7.13 (m, 1H), 7.03-6.97 (m, 1H), 6.34-6.27 (m, 1H), 5.02 (q, J=7.2 Hz, 1H), 4.89 (br s, 1H), 4.78 (br d, J=9.6 Hz, 1H), 4.69 (br d, J=12.0 Hz, 2H), 4.61-4.56 (m, 1H), 4.47-4.32 (m, 5H), 4.22-4.14 (m, 1H), 3.95-3.77 (m, 6H), 3.60 (br d, J=12.4 Hz, 1H), 3.54-3.47 (m, 1H), 2.47-1.77 (m, 21H), 1.58-1.49 (m, 3H), 1.06-1.02 (m, 3H), 0.91-0.81 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 105)

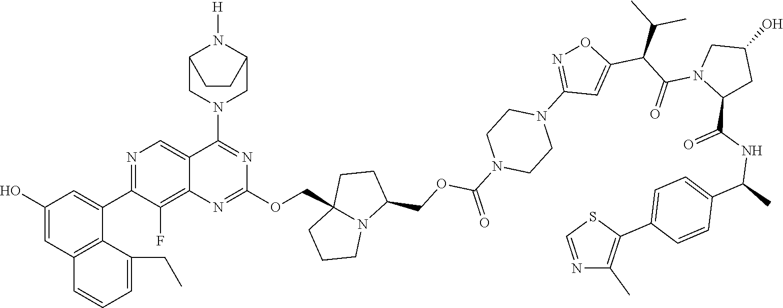 [SMILES: CCc1cccc2cc(O)cc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 87 starting from (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide, and purified by prep-HPLC (column: Phenomenex C18 75*30 mm*3 um; mobile phase: [water (FA)-ACN]; B %: 5%-45%, Gradient Time: 28 min, Flow Rate: 25 mL/min) to afford [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (43.7 mg, 18%, FA) as a white solid. MS (ESI) m/z: 1191.5 [M+H]⁺; ¹H NMR (400 MHz, CD₃OD) δ 9.12 (d, J=10.0 Hz, 1H), 8.87 (s, 1H), 7.63 (d, J=8.0 Hz, 1H), 7.44-7.34 (m, 6H), 7.31-7.29 (m, 1H), 7.17-7.14 (m, 1H), 7.04-7.02 (m, 1H), 6.08-6.07 (m, 1H), 5.05-5.00 (m, 1H), 4.53-4.42 (m, 4H), 4.31-4.27 (m, 1H), 4.18-4.14 (m, 1H), 4.04-4.01 (m, 2H), 3.90-3.81 (m, 3H), 3.66-3.48 (m, 6H), 3.15 (s, 6H), 2.47 (s, 3H), 2.39-2.27 (m, 5H), 2.15-1.91 (m, 14H), 1.51-1.49 (m, 3H), 1.06-1.04 (m, 3H), 0.90-0.86 (m, 6H).

Exemplary Synthesis of [(3R,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 106)

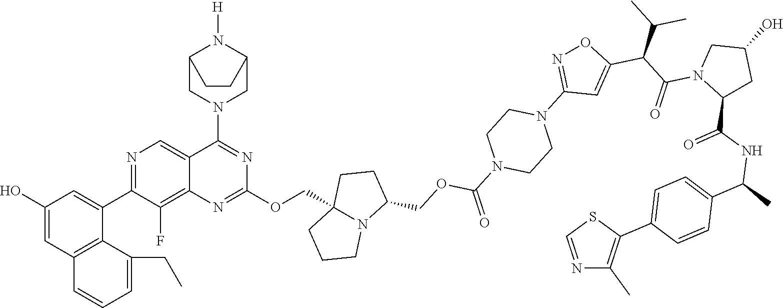 [SMILES: CCc1cccc2cc(O)cc(-c3ncc4c(N5CC6CCC(C5)N6)nc(OC[C@@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 87 starting from [(3R,8S)-3-[[tert-butyl(diphenyl)silyl]oxymethyl]-1,2,3,5,6,7-hexahydropyrrolizin-8-yl]methanol, and purified by prep-HPLC (column: Phenomenex C18 75*30 mm*3 um; mobile phase: [water (FA)-ACN]; B %: 18%-58%, Gradient Time: 26 min, Flow Rate: 25 mL/min) to [(3R,7aS)-7a-{[(4-{3,8-diazabicyclo[3.2.1]octan-3-yl}-7-(8-ethyl-3-afford hydroxynaphthalen-1-yl)-8-fluoropyrido[4,3-d]pyrimidin-2-yl)oxy]methyl}-hexahydro-1H-pyrrolizin-3-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (130.7 mg, 15%, FA) as a white solid. MS (ESI) m/z: 1191.4 [M+H]⁺; ¹H NMR (400 MHz, CD₃OD) δ 9.11 (s, 1H), 8.88 (s, 1H), 7.65-7.61 (m, 1H), 7.44-7.37 (m, 5H), 7.30-7.28 (m, 1H), 7.17-7.16 (m, 1H), 7.03-7.02 (m, 1H), 6.10-6.07 (m, 1H), 5.06-5.04 (m, 1H), 4.81-4.72 (m, 2H), 4.52-4.42 (m, 4H), 4.37-4.31 (m, 1H), 4.21-4.15 (m, 1H), 4.08-4.01 (m, 2H), 3.91-3.80 (m, 3H), 3.67-3.64 (m, 1H), 3.54-3.45 (m, 6H), 3.23-3.08 (m, 5H), 2.48 (s, 3H), 2.37-2.16 (m, 7H), 2.09-1.94 (m, 11H), 1.52-1.50 (m, 3H), 1.06-1.04 (m, 3H), 0.90-0.87 (m, 6H).

Exemplary Synthesis of (2S,4R)-1-[(2R)-2-(3-{7-[(2R)-2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]propyl]-2,7-diazaspiro[3.5]nonan-2-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 25)

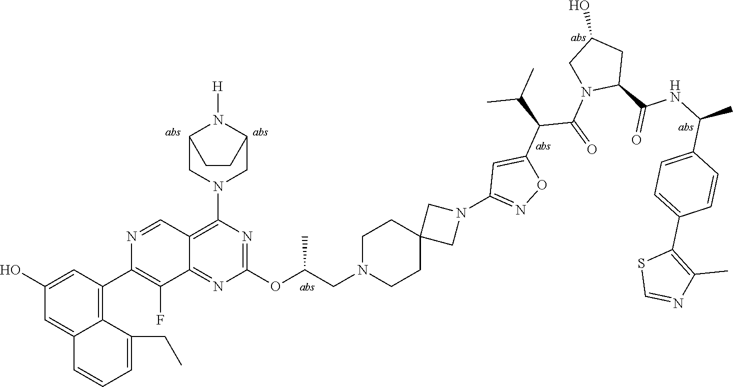 [SMILES: CCc1cccc2cc(O)cc(-c3ncc4c(N5CC6CCC(C5)N6)nc(O[C@H](C)CN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12]

The title compound was prepared in an analogous manner to Compound 101 starting from tert-butyl 3-[7-(8-ethyl-3-hydroxy-1-naphthyl)-8-fluoro-2-[(1R)-1-methyl-2-oxo-ethoxy]pyrido[4,3-d]pyrimidin-4-yl]-3,8-diazabicyclo[3.2.1]octane-8-carboxylate, and purified by prep-HPLC (column: Phenomenex C18 75*30 mm*3 um; mobile phase: [water (FA)-ACN]; B %: 10%-50%, 28 min) to afford (2S,4R)-1-[(2R)-2-(3-{7-[(2R)-2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]propyl]-2,7-diazaspiro[3.5]nonan-2-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (36.7 mg, 46%) as a white solid. MS (ESI) m/z: 1093.0 [M+H]⁺; ¹H NMR (400 MHz, CD₃OD) δ 9.08 (d, J=2.8 Hz, 1H), 8.91-8.84 (m, 1H), 8.42 (s, 1H), 7.69-7.58 (m, 1H), 7.48-7.33 (m, 5H), 7.31-7.27 (m, 1H), 7.17 (d, J=7.2 Hz, 1H), 7.01 (t, J=3.2 Hz, 1H), 5.88-5.77 (m, 1H), 5.73-5.63 (m, 1H), 5.03 (q, J=7.2 Hz, 1H), 4.76-4.68 (m, 3H), 4.53-4.38 (m, 2H), 4.03 (d, J=8.0 Hz, 2H), 3.91-3.78 (m, 3H), 3.68-3.59 (m, 5H), 3.08-2.99 (m, 1H), 2.95-2.70 (m, 5H), 2.51-2.44 (m, 3H), 2.39-2.26 (m, 3H), 2.22-2.14 (m, 1H), 2.09-1.95 (m, 5H), 1.93-1.82 (m, 4H), 1.59-1.49 (m, 3H), 1.47-1.40 (m, 3H), 1.04 (d, J=6.4 Hz, 3H), 0.94-0.84 (m, 6H).

Exemplary Synthesis of (2S,4R)-1-[(2R)-2-(3-{[(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 27)

Step 1: Preparation of methyl 3-[[tert-butyl(dimethyl)silyl]oxymethyl]-1,2,3,5,6,7-hexahydropyrrolizine-8-carboxylate

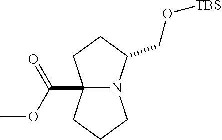

To a solution of methyl 3-(hydroxymethyl)-1,2,3,5,6,7-hexahydropyrrolizine-8-carboxylate (38.0 g, 190.72 mmol, 1.0 eq) and TBSCl (34.5 g, 228.86 mmol, 28.0 mL, 1.2 eq) in CH₂Cl₂ (1000 mL) was added IMIDAZOLE (16.9 g, 247.93 mmol, 1.3 eq). The mixture was stirred at 20° C. for 15 hours. The reaction mixture was quenched by addition of water (200 mL) and extracted with CH₂Cl₂ (300 mL). The combined organic layer was washed with brine (100 mL*2), dried over anhydrous Na₂SO₄, filtered and concentrated under reduced pressure to give a residue. The residue was purified by Biotage® combi flash (Column: 330 g SepaFlash® Silica Flash column; Eluent: gradient 0˜15% THF in petroleum ether, Gradient time: 25 min; Hold time: 20 min, Flow rate: 100 mL/min) to afford methyl 3-[[tert-butyl(dimethyl)silyl]oxymethyl]-1,2,3,5,6,7-hexahydropyrrolizine-8-carboxylate (20.2 g, 17%) as yellow oil. ¹H NMR (400 MHz, CDCl₃) δ 3.85-3.82 (m, 1H), 3.75-3.71 (m, 1H), 3.66 (s, 3H), 3.28-3.23 (m, 1H), 2.91-2.88 (m, 1H), 2.81-2.77 (m, 1H), 2.43-2.38 (m, 1H), 2.15-2.08 (m, 1H), 1.83-1.55 (m, 6H), 0.83 (s, 9H), 0.00 (s, 6H).

Step 2: Preparation of [3-[[tert-butyl(diphenyl)silyl]oxymethyl]-1,2,3,5,6,7-hexahydropyrrolizin-8-yl]methanol

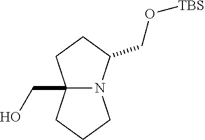

To a solution of methyl 3-[[tert-butyl(dimethyl)silyl]oxymethyl]-1,2,3,5,6,7-hexahydropyrrolizine-8-carboxylate (26.2 g, 83.57 mmol, 1.0 eq) in THF (400 mL) was added LiAlH₄ (3.5 g, 91.93 mmol, 1.1 eq) at 0° C. The mixture was stirred at 0° C. for 0.5 hour. The mixture was quenched by water (3.5 mL), NaOH (15%, 3.5 mL) and water (10.5 mL). Then mixture was diluted with ethyl acetate (100 mL) and dried over Na₂SO₄, filtered and concentrated under reduced pressure to afford [3-[[tert-butyl(dimethyl)silyl]oxymethyl]-1,2,3,5,6,7-hexahydropyrrolizin-8-yl]methanol (23.3 g, crude) as a light-yellow oil. H NMR (400 MHz, CDCl₃) δ 3.82-3.78 (m, 1H), 3.68-3.64 (m, 1H), 3.27-3.24 (m, 2H), 3.23-3.07 (m, 1H), 2.79-2.78 (m, 1H), 2.69-2.67 (m, 1H), 1.91-1.71 (m, 1H), 1.69-1.48 (m, 7H), 0.83 (s, 9H), 0.00 (s, 6H).

Step 3: Preparation of tert-butyl-[[3-[[tert-butyl(dimethyl)silyl]oxymethyl]-1,2,3,5,6,7-hexahydropyrrolizin-8-yl]methoxy]-diphenyl-silane

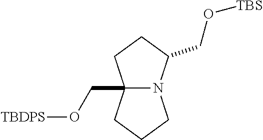

To a solution of [3-[[tert-butyl(dimethyl)silyl]oxymethyl]-1,2,3,5,6,7-hexahydropyrrolizin-8-yl]methanol (23.3 g, 81.61 mmol, 1.0 eq) in CH₂Cl₂ (400 mL) was added imidazole (7.2 g, 106.10 mmol, 1.3 eq) and TBDPSCl (26.9 g, 97.93 mmol, 1.2 eq). The mixture was stirred at 15° C. for 15 hours. The combined organic layers were dried over Na₂SO₄, filtered and concentrated under reduced pressure. The residue was purified by flash column chromatography (0˜10% THF in petroleum ether) to afford tert-butyl-[[3-[[tert-butyl(dimethyl)silyl]oxymethyl]-1,2,3,5,6,7-hexahydropyrrolizin-8-yl]methoxy]-diphenyl-silane (27 g, 51%) as a light-yellow oil. MS (ESI) m/z: 524.7 [M+H]⁺.

Step 4: Preparation of tert-butyl-[[(3R,8R)-3-[[tert-butyl(dimethyl)silyl]oxymethyl]-1,2,3,5,6,7-hexahydropyrrolizin-8-yl]methoxy]-diphenyl-silane and tert-butyl-[[(3S,8S)-3-[[tert-butyl(dimethyl)silyl]oxymethyl]-1,2,3,5,6,7-hexahydropyrrolizin-8-yl]methoxy]-diphenyl-silane

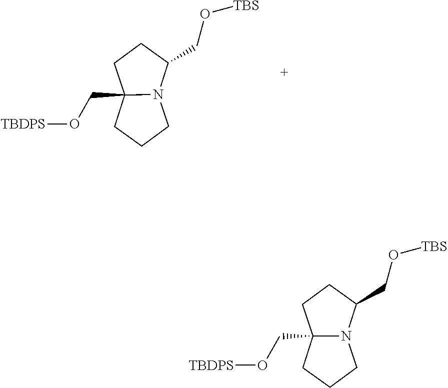

The mixture was purified by SFC (column: REGIS (s,s) WHELK-01 (250 mm*50 mm, 10 um); mobile phase: [0.1% NH₃H₂O IPA]; B %: 40%-40%) to afford tert-butyl-[[(3R,8R)-3-[[tert-butyl(dimethyl)silyl]oxymethyl]-1,2,3,5,6,7-hexahydropyrrolizin-8-yl]methoxy]-diphenyl-silane (10.7 g, 34%) as a light-yellow oil, and tert-butyl-[[(3S,8S)-3-[[tert-butyl(dimethyl)silyl]oxymethyl]-1,2,3,5,6,7-hexahydropyrrolizin-8-yl]methoxy]-diphenyl-silane (11.1 g, 35%) as a yellow oil. MS (ESI) m/z: 524.7 [M+H]⁺.

Step 5: Preparation of [(3R,8R)-8-[[tert-butyl(diphenyl)silyl]oxymethyl]-1,2,3,5,6,7-hexahydropyrrolizin-3-yl]methanol

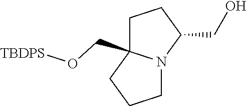

To a mixture of tert-butyl-[[(3R,8R)-3-[[tert-butyl(dimethyl)silyl]oxymethyl]-1,2,3,5,6,7-hexahydropyrrolizin-8-yl]methoxy]-diphenyl-silane (2.0 g, 3.82 mmol, 1.0 eq) in CH₂Cl₂ (20 mL) was added HCl/dioxane (4.0 M, 20.0 mL, 21.0 eq) at 20° C., the resulting suspension was stirred at 20° C. for 1 hour. The reaction mixture was concentrated under reduced pressure, the residue was diluted with water (10 mL), adjusted the pH to 8 by sat. NaHCO₃ solution. The mixture was extracted with CH₂Cl₂ (30 mL×3). The organic layers were combined, dried over Na₂SO₄, filtered, and concentrated under reduced pressure. The residue was purified by flash column chromatography (0-40% Ethyl acetate in petroleum ether) to afford [(3R,8R)-8-[[tert-butyl(diphenyl)silyl]oxymethyl]-1,2,3,5,6,7-hexahydropyrrolizin-3-yl]methanol (1.57 g, 82%) as a colorless oil. MS (ESI) m/z: 410.1 [M+H]⁺.

Step 6: Preparation of methyl 2-[3-[[(3R,8R)-8-[[tert-butyl(diphenyl)silyl]oxymethyl]-1,2,3,5,6,7-hexahydropyrrolizin-3-yl]methoxy]isoxazol-5-yl]-3-methyl-butanoate

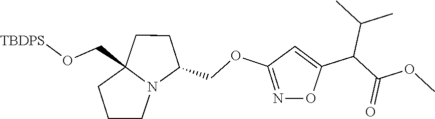

To a reaction mixture of [(3R,8R)-8-[[tert-butyl(diphenyl)silyl]oxymethyl]-1,2,3,5,6,7-hexahydropyrrolizin-3-yl]methanol (1.57 g, 3.83 mmol, 1.0 eq) and methyl 2-(3-hydroxyisoxazol-5-yl)-3-methyl-butanoate (763.48 mg, 3.83 mmol, 1.0 eq) in toluene (20 mL) were added PPh₃ (2.01 g, 7.67 mmol, 2.0 eq). The mixture was cooled to 0° C., then DIAD (1.49 mL, 7.67 mmol, 2.0 eq) was added, the resulting suspension was stirred at 110° C. for 15 hours under N₂. The reaction mixture was concentrated under reduced pressure. The residue was purified by flash column chromatography (0-10% ethyl acetate in petroleum ether) to afford methyl 2-[3-[[(3R,8R)-8-[[tert-butyl(diphenyl)silyl]oxymethyl]-1,2,3,5,6,7-hexahydropyrrolizin-3-yl]methoxy]isoxazol-5-yl]-3-methyl-butanoate (1.76 g, 53%) as a yellow oil. MS (ESI) m/z: 591.3 [M+H]⁺.

Step 7: Preparation of methyl 2-[3-[[(3R,8R)-8-(hydroxymethyl)-1,2,3,5,6,7-hexahydropyrrolizin-3-yl]methoxy]isoxazol-5-yl]-3-methyl-butanoate

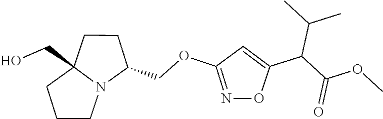 [SMILES: COC(=O)C(c1cc(OC[C@H]2CC[C@@]3(CO)CCCN23)no1)C(C)C]

To a solution of methyl 2-[3-[[(3R,8R)-8-[[tert-butyl(diphenyl)silyl]oxymethyl]-1,2,3,5,6,7-hexahydropyrrolizin-3-yl]methoxy]isoxazol-5-yl]-3-methyl-butanoate (1.76 g, 2.23 mmol, 75% purity, 1.0 eq) in CH₂Cl₂ (25 mL) was added N,N-diethylethanamine; trihydrofluoride (5.46 mL, 33.51 mmol, 15 eq), the resulting suspension was stirred at 20° C. for 1 hour under N₂. The reaction mixture was concentrated under reduced pressure. The crude product was purified by prep-HPLC (column: YMC Triart C18 250*50 mm*7 um; mobile phase: [water (HCl)-ACN]) to afford methyl 2-[3-[[(3R,8R)-8-(hydroxymethyl)-1,2,3,5,6,7-hexahydropyrrolizin-3-yl]methoxy]isoxazol-5-yl]-3-methyl-butanoate (700 mg, 74%, HCl) as a pink oil. MS (ESI) m/z: 353.3 [M+H]⁺.

Step 8: Preparation of tert-butyl 3-[7-[8-ethyl-3-(methoxymethoxy)-1-naphthyl]-8-fluoro-2-[[(3R,8R)-3-[[5-(1-methoxycarbonyl-2-methylpropyl)isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

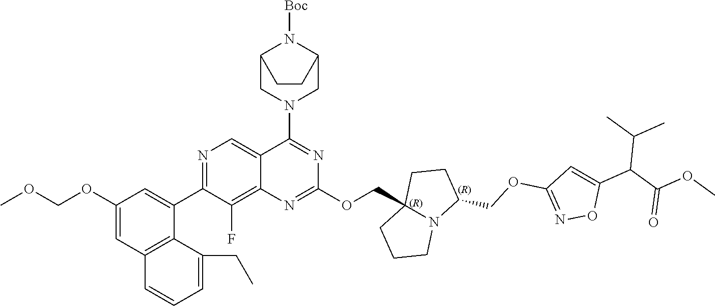

To a reaction mixture of methyl 2-[3-[[(3R,8R)-8-(hydroxymethyl)-1,2,3,5,6,7-hexahydropyrrolizin-3-yl]methoxy]isoxazol-5-yl]-3-methyl-butanoate (300 mg, 771.44 umol, 1.26 eq, HCl) in dioxane (10 mL) was added DIEA (160 μL, 920.60 umol, 1.5 eq). Then tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (400 mg, 613.74 umol, 1.0 eq) and 4 A MS (400 mg) was added. The reaction mixture was cooled to 0° C., then t-BuONa (176.94 mg, 1.84 mmol, 3.0 eq) was added. The resulting suspension was stirred at 10° C. for 30 min. The reaction mixture was quenched with HCl (2 M) to pH 4 and extracted with EtOAc (10 mL×2). The organic layers were combined, dried over Na₂SO₄, filtered and concentrated under reduced pressure. The residue was purified by flash column chromatography (20˜40% ethyl acetate in petroleum ether) to afford tert-butyl 3-[7-[8-ethyl-3-(methoxymethoxy)-1-naphthyl]-8-fluoro-2-[[(3R,8R)-3-[[5-(1-methoxycarbonyl-2-methylpropyl)isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (636 mg, 50%) as a yellow oil. MS (ESI) m/z: 924.3 [M+H]⁺.

Step 9: Preparation of 2-[3-[[(3R,8R)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoic acid

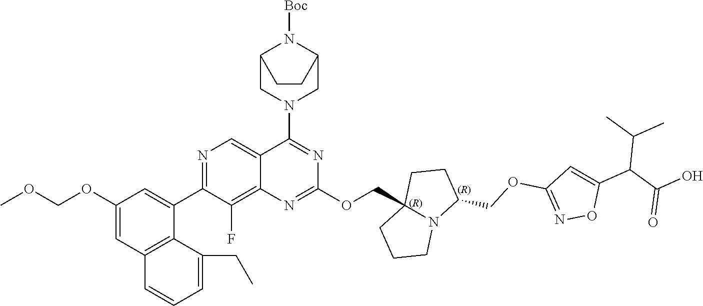

A mixture of tert-butyl 3-[7-[8-ethyl-3-(methoxymethoxy)-1-naphthyl]-8-fluoro-2-[[(3R,8R)-3-[[5-(1-methoxycarbonyl-2-methyl-propyl)isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (300 mg, 324.65 umol, 1.0 eq) in a solution of THF (5 mL) was added a solution of LiOH·H₂O (40.87 mg, 973.96 umol, 3.0 eq) in H₂O (2.5 mL) and then the mixture was stirred at 15° C. for 16 hours. The mixture was diluted with water (5 mL), adjusted the pH to 4 with aq. HCl (1 N), then extracted with MeOH/dichloromethane (30 mL×3, 1/20). The combined organic phase was washed with brine (20 mL), dried over anhydrous sodium sulfate, filtered and concentrated under reduced pressure to afford 2-[3-[[(3R,8R)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoic acid (318 mg, crude) as a yellow solid, which was used directly in the next step without further purification. MS (ESI) m/z: 910.6 [M+H]⁺.

Step 10: Preparation of tert-butyl 3-[7-[8-ethyl-3-(methoxymethoxy)-1-naphthyl]-8-fluoro-2-[[(3R,8R)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

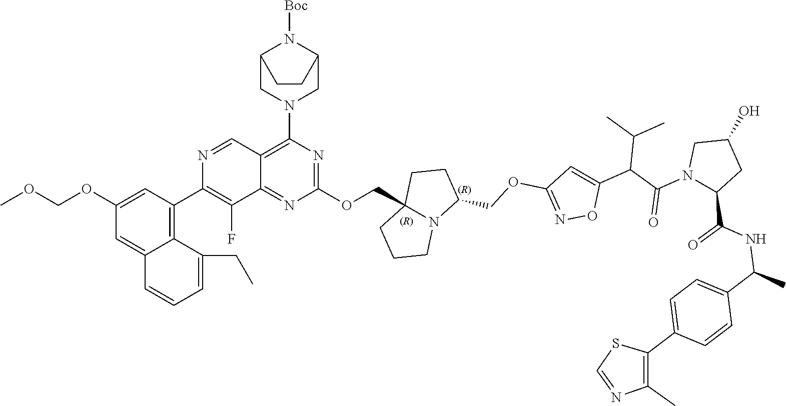

A mixture of 2-[3-[[(3R,8R)-8-[[4-(8-tert-butoxycarbonyl-3,8-diazabicyclo[3.2.1]octan-3-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoic acid (280 mg, 233.84 umol, 1.0 eq), (2S,4R)-4-hydroxy-N-[(1S)-1-[4-(4-methylthiazol-5-yl)phenyl]ethyl]pyrrolidine-2-carboxamide (128.40 mg, 233.84 umol, 1.0 eq, HCl), DIEA (204 uL, 1.17 mmol, 5 eq) in DMF (4 mL) was added HATU (93.36 mg, 245.53 umol, 1.05 eq), and then the mixture was stirred at 15° C. for 2 hours. To the reaction mixture was added water (40 mL), filtered, and the filter cake was washed with water (5 mL×2), then the solid material was diluted with dichloromethane (50 mL). The combined organic phase was dried over anhydrous sodium sulfate, filtered and concentrated under reduced pressure. The residue was purified by flash column chromatography (0˜100% ethyl acetate in petroleum ether) to afford tert-butyl 3-[7-[8-ethyl-3-(methoxymethoxy)-1-naphthyl]-8-fluoro-2-[[(3R,8R)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (190 mg, 55%) as a yellow solid. MS (ESI) m/z: 612.5 [½M+H]⁺.

Step 11: Preparation of tert-butyl 3-[7-[8-ethyl-3-(methoxymethoxy)-1-naphthyl]-8-fluoro-2-[[(3R,8R)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate and tert-butyl 3-[7-[8-ethyl-3-(methoxymethoxy)-1-naphthyl]-8-fluoro-2-[[(3R,8R)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

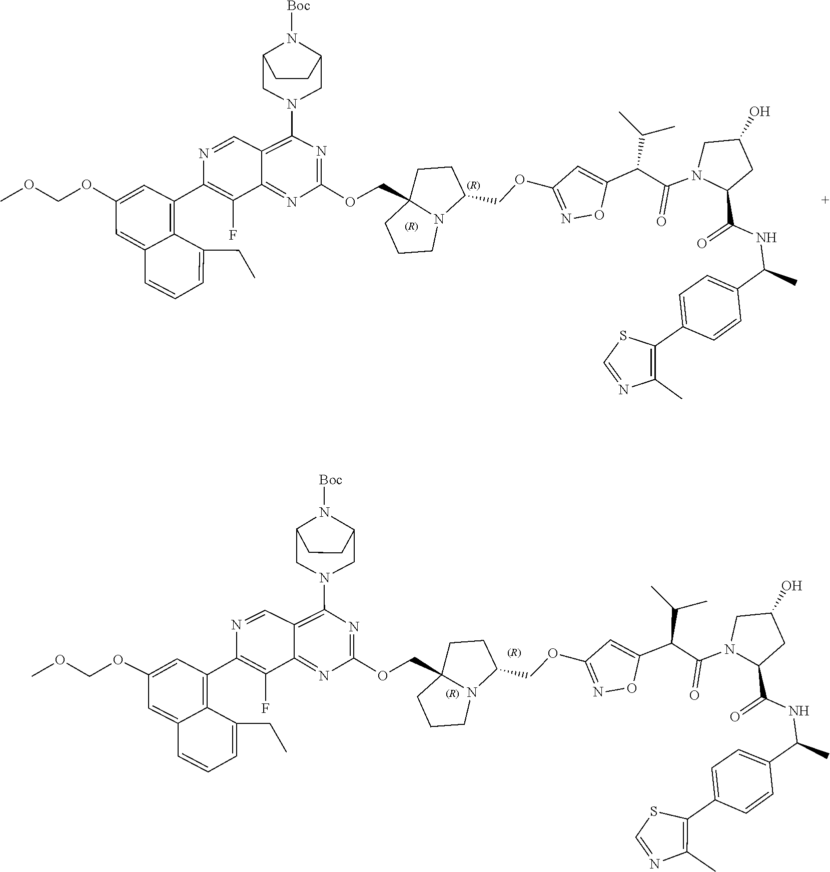

Tert-butyl 3-[7-[8-ethyl-3-(methoxymethoxy)-1-naphthyl]-8-fluoro-2-[[(3R,8R)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (430 mg, 350.63 umol) was purified by SFC (column: DAICEL CHIRALPAKIA (250 mm*30 mm, 10 um); mobile phase: [ACN/EtOH (0.1% NH₃H₂O)]; B %: 30%-30%) to afford tert-butyl 3-[7-[8-ethyl-3-(methoxymethoxy)-1-naphthyl]-8-fluoro-2-[[(3R,8R)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (205 mg, 26% yield) as a yellow solid, MS (ESI) m/z: 613.0 [½M+H]⁺; and tert-butyl 3-[7-[8-ethyl-3-(methoxymethoxy)-1-naphthyl]-8-fluoro-2-[[(3R,8R)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (250 mg, 33%) as a yellow solid. MS (ESI) m/z: 613.0 [½M+H]⁺.

Step 12: Preparation of (2S,4R)-1-[(2R)-2-[3-[[(3R,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

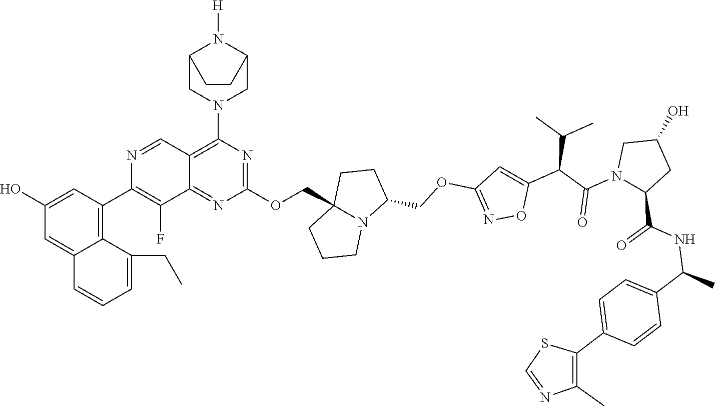 [SMILES: CCc1cccc2cc(O)cc(-c3ncc4c(N5CC6CCC(C5)N6)nc(OC[C@]56CCCN5[C@@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

A reaction mixture of tert-butyl 3-[7-[8-ethyl-3-(methoxymethoxy)-1-naphthyl]-8-fluoro-2-[[(3R,8R)-3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (150 mg, 122.60 umol, 1.0 eq) in HCOOH (15 mL) was stirred at 20° C. for 2 hours. The reaction mixture was concentrated under reduced pressure. The pH of the residue was adjusted to 8 by sat. aq. NaHCO₃, the solvent was extracted with CH₂Cl₂/MeOH (30 mL×2, V:V=1:1), the organic layers were combined and concentrated under reduced pressure. The crude product was purified by prep-HPLC (column: Phenomenex C18 75*30 mm*3 um; mobile phase: [water (FA)-ACN]) to afford (2S,4R)-1-[(2R)-2-(3-{[(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (80.3 mg, 58%, FA) as a white solid. MS (ESI) m/z: 1079.8 [M+H]⁺; ¹H NMR (400 MHz, CD₃OD) δ 9.12 (d, J=2.0 Hz, 1H), 8.88 (s, 1H), 7.67-7.61 (m, 1H), 7.48-7.33 (m, 5H), 7.33-7.28 (m, 1H), 7.17 (br d, J=6.8 Hz, 1H), 7.02 (t, J=2.8 Hz, 1H), 6.04-5.97 (m, 1H), 5.03 (q, J=7.2 Hz, 1H), 4.80 (br d, J=14.8 Hz, 2H), 4.74-4.67 (m, 2H), 4.64-4.35 (m, 6H), 4.00-3.77 (m, 5H), 3.68 (t, J=9.2 Hz, 1H), 3.60 (br d, J=11.2 Hz, 1H), 3.47 (br d, J=9.6 Hz, 1H), 3.41-3.34 (m, 1H), 2.50-2.45 (m, 3H), 2.43-2.31 (m, 4H), 2.28-2.09 (m, 6H), 2.06-1.92 (m, 6H), 1.61-1.48 (m, 3H), 1.04 (br dd, J=4.2, 6.0 Hz, 3H), 0.93-0.80 (m, 6H).

Exemplary Synthesis of (2S,4R)-1-[(2S)-2-(3-{[(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 28)

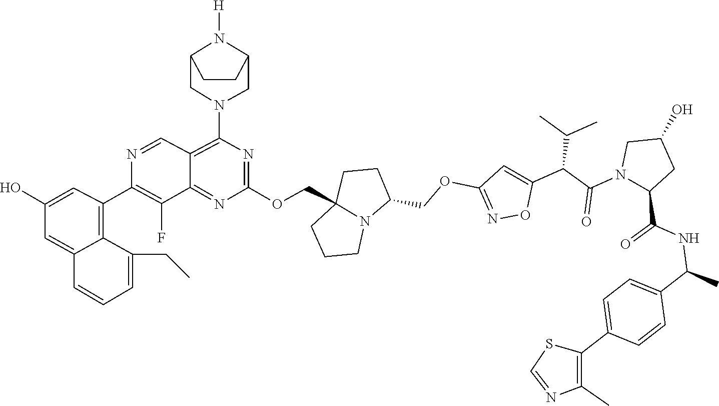 [SMILES: CCc1cccc2cc(O)cc(-c3ncc4c(N5CC6CCC(C5)N6)nc(OC[C@]56CCCN5[C@@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 27 starting from tert-butyl 3-[7-[8-ethyl-3-(methoxymethoxy)-1-naphthyl]-8-fluoro-2-[[(3R,8R)-3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate, and purified by prep-HPLC (column: Phenomenex C18 75*30 mm*3 um; mobile phase: [water (FA)-ACN]) to afford (2S,4R)-1-[(2S)-2-(3-{[(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (32.8 mg, 24%, FA) as a white solid. MS (ESI) m/z: 1079.8 [M+H]⁺; ¹H NMR (400 MHz, CD₃OD) δ 9.10 (s, 1H), 8.90 0-8.83 (m, 1H), 7.60 (br d, J=8.4 Hz, 1H), 7.40-7.23 (m, 6H), 7.18-7.11 (m, 1H), 7.02 (dd, J=2.4, 6.8 Hz, 1H), 6.05-5.99 (m, 1H), 4.76-4.24 (m, 11H), 3.87-3.54 (m, 8H), 3.46-3.39 (m, 1H), 2.50-2.29 (m, 8H), 2.24-2.16 (m, 3H), 2.13-2.01 (m, 4H), 1.94-1.84 (m, 4H), 1.43 (dd, J=7.2, 18.4 Hz, 3H), 1.09-1.00 (m, 3H), 0.93-0.85 (m, 6H).

Exemplary Synthesis of (2S,4R)-1-[(2R)-2-(3-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 29)

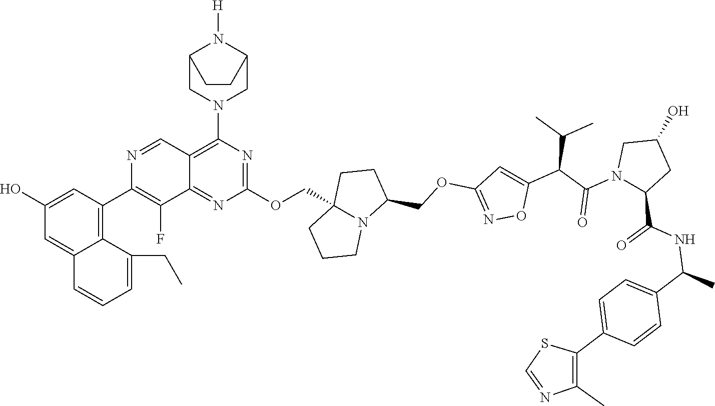 [SMILES: CCc1cccc2cc(O)cc(-c3ncc4c(N5CC6CCC(C5)N6)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 27 starting from tert-butyl-[[(3S,8S)-3-[[tert-butyl(dimethyl)silyl]oxymethyl]-1,2,3,5,6,7-hexahydropyrrolizin-8-yl]methoxy]-diphenyl-silane, and purified by prep-HPLC (Condition: [water (FA)-ACN]; B %: 9%-39%, Gradient time: 25 min; Column: Phenomenex C18 75*30 mm*3 um; Flow Rate: 25 mL/min) to afford (2S,4R)-1-[(2R)-2-(3-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (76.6 mg, 56%, FA) as a white solid. MS (ESI) m/z: 1079.9 [M+1]⁺; ¹H NMR (400 MHz, CD₃OD) δ 9.15-9.09 (m, 1H), 8.91-8.84 (m, 1H), 8.49 (s, 1H), 7.68-7.60 (m, 1H), 7.48-7.27 (m, 6H), 7.17 (d, J=7.2 Hz, 1H), 7.05-6.99 (m, 1H), 6.03-5.98 (m, 1H), 5.06-4.98 (m, 1H), 4.82-4.72 (m, 2H), 4.68 (dd, J=2.8, 12.0 Hz, 2H), 4.63-4.46 (m, 4H), 4.45-4.27 (m, 2H), 3.95-3.74 (m, 5H), 3.73-3.64 (m, 1H), 3.58 (dd, J=4.0, 10.8 Hz, 1H), 3.51-3.45 (m, 1H), 3.35 (d, J=6.4 Hz, 1H), 2.51-2.43 (m, 3H), 2.42-2.31 (m, 4H), 2.29-2.09 (m, 6H), 2.04-1.88 (m, 6H), 1.60-1.48 (m, 3H), 1.05 (d, J=6.4 Hz, 3H), 0.94-0.81 (m, 6H).

Exemplary Synthesis of (2S,4R)-1-[(2S)-2-(3-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 30)

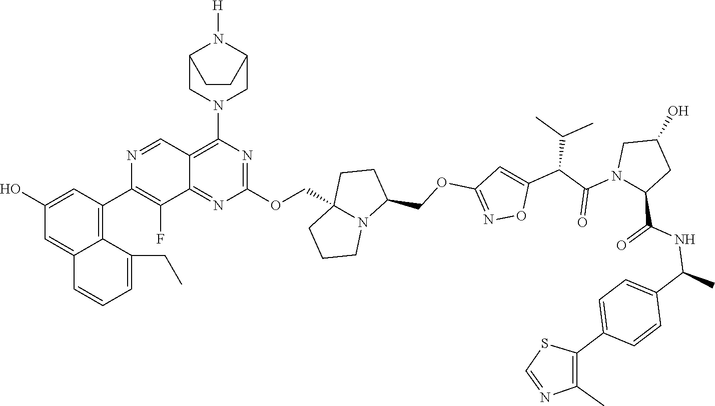 [SMILES: CCc1cccc2cc(O)cc(-c3ncc4c(N5CC6CCC(C5)N6)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 27 starting from tert-butyl-[[(3S,8S)-3-[[tert-butyl(dimethyl)silyl]oxymethyl]-1,2,3,5,6,7-hexahydropyrrolizin-8-yl]methoxy]-diphenyl-silane, and purified by prep-HPLC (Condition: [water (FA)-ACN]; B %: 8%-48%, Gradient time: 28 min; Column: Phenomenex C18 75*30 mm*3 um; Flow Rate: 25 mL/min) to afford (2S,4R)-1-[(2S)-2-(3-{[(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (55.6 mg, 46, FA) as a white solid. MS (ESI) m/z: 1079.7 [M+1]⁺; 1H NMR (400 MHz, CD₃OD) δ 9.10 (d, J=2.4 Hz, 1H), 8.90-8.84 (m, 1H), 8.52 (s, 1H), 7.63 (d, J=8.0 Hz, 1H), 7.47-7.36 (m, 3H), 7.36-7.32 (m, 2H), 7.30 (d, J=2.8 Hz, 1H), 7.16 (dd, J=2.8, 6.8 Hz, 1H), 7.02 (d, J=2.4 Hz, 1H), 6.04-5.99 (m, 1H), 4.96-4.91 (m, 1H), 4.74 (d, J=13.2 Hz, 1H), 4.65 (dd, J=4.0, 12.0 Hz, 2H), 4.61-4.45 (m, 5H), 4.41 (d, J=1.6 Hz, 1H), 4.27 (d, J=3.2 Hz, 1H), 3.88-3.57 (m, 8H), 3.39 (s, 1H), 2.49-2.44 (m, 3H), 2.39-2.31 (m, 4H), 2.27-2.16 (m, 3H), 2.16-2.06 (m, 3H), 2.01-1.94 (m, 3H), 1.92 (s, 3H), 1.46 (dd, J=2.8, 7.2 Hz, 3H), 1.05 (dd, J=3.6, 6.4 Hz, 3H), 0.89 (t, J=7.2 Hz, 6H).

Exemplary Synthesis of (2S,4R)-1-[(2S)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-methylpiperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 31)

Step 1: Preparation of tert-butyl 4-[[5-(1-methoxycarbonyl-2-methyl-propyl)isoxazol-3-yl]oxymethyl]-4-methyl-piperidine-1-carboxylate

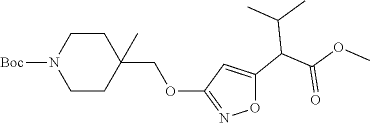 [SMILES: COC(=O)C(c1cc(OCC2(C)CCN(C(=O)OC(C)(C)C)CC2)no1)C(C)C]

To a reaction mixture of tert-butyl 4-(hydroxymethyl)-4-methyl-piperidine-1-carboxylate (1.0 g, 4.36 mmol, 1.0 eq) and methyl 2-(3-hydroxyisoxazol-5-yl)-3-methyl-butanoate (868 mg, 4.36 mmol, 1.0 eq) and DIAD (1.2 mL, 6.54 mmol, 1.5 eq) in THF (20 mL) was added PPh₃ (1.7 g, 6.54 mmol, 1.5 eq) at 0° C., and the reaction mixture was stirred at 60° C. for 12 hours. The mixture was concentrated under reduced pressure. The residue was purified by flash column chromatography (0˜5% tetrahydrofuran in petroleum ether) to afford tert-butyl 4-[[5-(1-methoxycarbonyl-2-methyl-propyl)isoxazol-3-yl]oxymethyl]-4-methyl-piperidine-1-carboxylate (1.1 g, 61%) as a colorless oil. MS (ESI) m/z: 311.2 [M+H−100]⁺.

Step 2: Preparation of 2-[3-[(1-tert-butoxycarbonyl-4-methyl-4-piperidyl)methoxy]isoxazol-5-yl]-3-methyl-butanoic acid

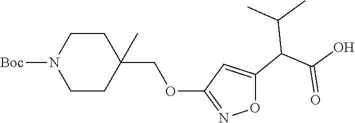 [SMILES: CC(C)C(C(=O)O)c1cc(OCC2(C)CCN(C(=O)OC(C)(C)C)CC2)no1]

To a reaction mixture of tert-butyl 4-[[5-(1-methoxycarbonyl-2-methyl-propyl)isoxazol-3-yl]oxymethyl]-4-methyl-piperidine-1-carboxylate (1.1 g, 2.75 mmol, 1.0 eq) in water (6 mL) and THF (6 mL) was added LiOH·H₂O (577 mg, 13.76 mmol, 5.0 eq), the suspension was stirred at 25° C. for 15 hours. The reaction mixture was concentrated under reduced pressure, the residue was diluted with water (5 mL), and adjusted the pH with 2 M aq. HCl to 4˜5. Then lyophilized to give 2-[3-[(1-tert-butoxycarbonyl-4-methyl-4-piperidyl)methoxy]isoxazol-5-yl]-3-methyl-butanoic acid (1.1 g, crude) as a white solid. MS (ESI) m/z: 297.1 [M+H−100]⁺.

Step 3: Preparation of tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate

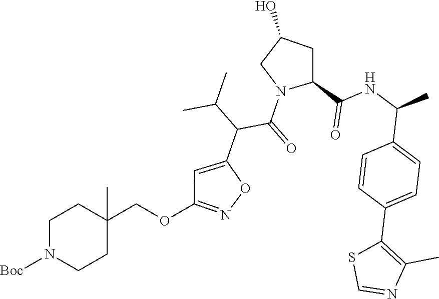 [SMILES: Cc1ncsc1-c1ccc([C@H](C)NC(=O)[C@@H]2C[C@@H](O)CN2C(=O)C(c2cc(OCC3(C)CCN(C(=O)OC(C)(C)C)CC3)no2)C(C)C)cc1]

To a solution of 2-[3-[(1-tert-butoxycarbonyl-4-methyl-4-piperidyl)methoxy]isoxazol-5-yl]-3-methyl-butanoic acid (1.1 g, 2.75 mmol, 1.0 eq) and DIEA (2 mL, 13.75 mmol, 5.0 eq) in CH₂Cl₂ (10 mL) was added HATU (1.3 g, 3.30 mmol, 1.2 eq) and the reaction mixture was stirred at 25° C. for 0.5 hour. Then (2S,4R)-4-hydroxy-N-[(1S)-1-[4-(4-methylthiazol-5-yl)phenyl]ethyl]pyrrolidine-2-carboxamide (911 mg, 2.75 mmol, 1.0 eq) was added, and the reaction mixture was stirred at 25° C. for 2 hours. The reaction mixture was diluted with water (20 mL). The organic layer was separated and the aqueous was extracted with dichloromethane (20 mL×2). The combined organic layers were washed with brine (20 mL×2), dried over Na₂SO₄, filtered and solvent was concentrated under reduced pressure. The residue was purified by flash column chromatography (0˜70% ethyl acetate in petroleum ether) to afford tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate (1.3 g, 1.75 mmol, 63.77% yield, 98% purity) as a yellow oil. MS (ESI) m/z: 710.4 [M+H]⁺.

Step 4: Preparation of tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate and tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate

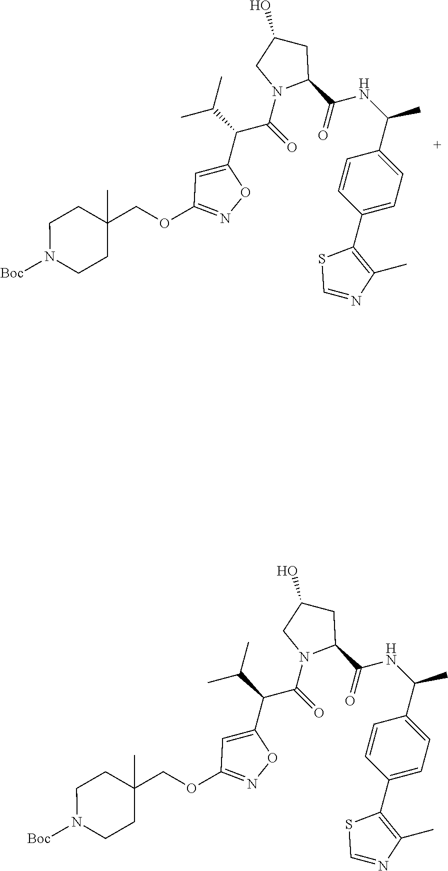 [SMILES: Cc1ncsc1-c1ccc([C@H](C)NC(=O)[C@@H]2C[C@@H](O)CN2C(=O)[C@H](c2cc(OCC3(C)CCN(C(=O)OC(C)(C)C)CC3)no2)C(C)C)cc1 ; Cc1ncsc1-c1ccc([C@H](C)NC(=O)[C@@H]2C[C@@H](O)CN2C(=O)[C@@H](c2cc(OCC3(C)CCN(C(=O)OC(C)(C)C)CC3)no2)C(C)C)cc1]

The tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate (1.2 g, 1.66 mmol) was separated by SFC (column: Welch Xtimate (250 mm*50 mm, 10 um); mobile phase: [0.1% NH₃H₂O ETOH]; Begin B: 45; End B: 45; 100% B; Flow Rate (ml/min): 140) to afford tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate (623 mg, 53%) as a yellow oil, and tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate (198 mg, 17%) as a white solid. MS (ESI) m/z: 710.4 [M+H]⁺.

Step 5: Preparation of (2S,4R)-4-hydroxy-1-[(2S)-3-methyl-2-[3-[(4-methyl-4-piperidyl)methoxy]isoxazol-5-yl]butanoyl]-N-[(1S)-1-[4-(4-methylthiazol-5-yl)phenyl]ethyl]pyrrolidine-2-carboxamide

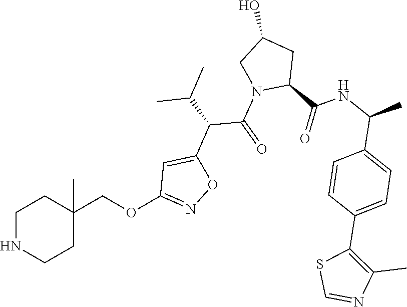 [SMILES: Cc1ncsc1-c1ccc([C@H](C)NC(=O)[C@@H]2C[C@@H](O)CN2C(=O)[C@H](c2cc(OCC3(C)CCNCC3)no2)C(C)C)cc1]

To a reaction mixture of tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate (200 mg, 278.92 umol, 1.0 eq) in CH₂Cl₂ (2 mL) was added HCl/dioxane (4 M, 1 mL, 14.3 eq) was stirred at 25° C. for 1 hour. The reaction mixture was concentrated under reduced pressure. Then the residue was diluted with water (5 mL). The pH of the resulting mixture was adjusted to 8 by sat. aq. NaHCO₃ and extracted with ethyl acetate (20 mL×3). The combined organic layer was washed with brine (20 mL), dried over Na₂SO₄, filtered, and concentrated under reduced pressure to afford (2S,4R)-4-hydroxy-1-[(2S)-3-methyl-2-[3-[(4-methyl-4-piperidyl)methoxy]isoxazol-5-yl]butanoyl]-N-[(1S)-1-[4-(4-methylthiazol-5-yl)phenyl]ethyl]pyrrolidine-2-carboxamide (124 mg, crude) as a colorless oil, which would be directly used in the next step. MS (ESI) m/z: 610.3 [M+H]⁺.

Step 6: Preparation of tert-butyl 3-[7-(8-ethyl-3-hydroxy-1-naphthyl)-8-fluoro-2-[2-[[4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate

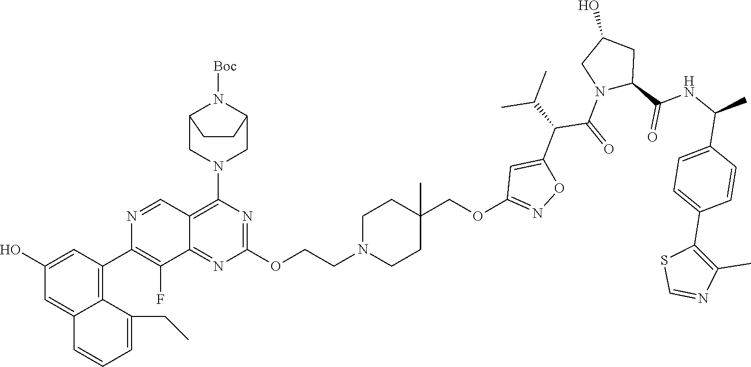 [SMILES: CCc1cccc2cc(O)cc(-c3ncc4c(N5CC6CCC(C5)N6C(=O)OC(C)(C)C)nc(OCCN5CCC(C)(COc6cc([C@@H](C(=O)N7C[C@H](O)C[C@H]7C(=O)N[C@@H](C)c7ccc(-c8scnc8C)cc7)C(C)C)on6)CC5)nc4c3F)c12]

To a reaction mixture of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (131 mg, 223.69 umol, 1.1 eq) and (2S,4R)-4-hydroxy-1-[(2S)-3-methyl-2-[3-[(4-methyl-4-piperidyl)methoxy]isoxazol-5-yl]butanoyl]-N-[(1S)-1-[4-(4-methylthiazol-5-yl)phenyl]ethyl]pyrrolidine-2-carboxamide (124 mg, 203.35 umol, 1.0 eq) in CH₂Cl₂ (2 mL) and i-PrOH (2 mL) were added AcOH (47 uL, 813.41 umol, 4.0 eq) and borane; 2-methylpyridine (108.75 mg, 1.02 mmol, 5.0 eq) and the resulting suspension was stirred at 25° C. for 1 hour. The reaction mixture was concentrated under reduced pressure. The residue was diluted with water (10 mL), adjusted the pH with saturated aqueous sodium bicarbonate solution to 8. The mixture was extracted with dichloromethane (20 mL×3). The combined organic extracts were washed with brine (20 mL×3), dried over Na₂SO₄, filtered and concentrated. The residue was purified by flash column chromatography (0˜10% ethyl acetate in petroleum ether) to afford tert-butyl 3-[7-(8-ethyl-3-hydroxy-1-naphthyl)-8-fluoro-2-[2-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate (175 mg, 50%) as a yellow solid. MS (ESI) m/z: 591.7 [M/2+H]⁺.

Step 7: Preparation of (2S,4R)-1-[(2S)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-methylpiperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

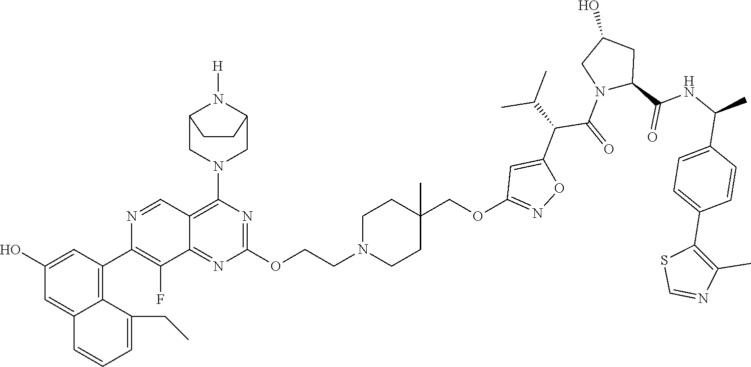 [SMILES: CCc1cccc2cc(O)cc(-c3ncc4c(N5CC6CCC(C5)N6)nc(OCCN5CCC(C)(COc6cc([C@@H](C(=O)N7C[C@H](O)C[C@H]7C(=O)N[C@@H](C)c7ccc(-c8scnc8C)cc7)C(C)C)on6)CC5)nc4c3F)c12]

A reaction mixture of tert-butyl 3-[7-(8-ethyl-3-hydroxy-1-naphthyl)-8-fluoro-2-[2-[[4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate (175 mg, 102.21 umol, 69% purity, 1.0 eq) in HCOOH (8 mL) was stirred at 20° C. for 1 hour. The reaction mixture was concentrated under reduced pressure. The crude product was purified by prep-HPLC (column: Welch Xtimate C18 250*50 mm*10 um; mobile phase: [water (FA)-ACN]) to afford (2S,4R)-1-[(2S)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-methylpiperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (58 mg, 52%) as a white solid. MS (ESI) m/z: 541.8 [M/2+H]⁺; ¹H NMR (400 MHz, CD₃OD) δ 9.09 (s, 1H), 8.89-8.84 (m, 1H), 7.66-7.60 (m, 1H), 7.43-7.31 (m, 5H), 7.31-7.27 (m, 1H), 7.16 (br d, J=6.8 Hz, 1H), 7.03-6.97 (m, 1H), 6.06-5.97 (m, 1H), 5.01-4.96 (m, 2H), 4.83-4.73 (m, 5H), 4.57 (t, J=8.0 Hz, 1H), 4.45-4.38 (m, 1H), 4.04-3.97 (m, 4H), 3.86-3.63 (m, 5H), 3.14-3.07 (m, 2H), 2.49-2.44 (m, 3H), 2.43-2.18 (m, 5H), 2.06-1.84 (m, 8H), 1.69-1.61 (m, 2H), 1.48 (d, J=7.2 Hz, 3H), 1.15-1.12 (m, 3H), 1.05 (d, J=6.8 Hz, 3H), 0.93-0.86 (m, 6H).

Exemplary Synthesis of (2S,4R)-1-[(2R)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-methylpiperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 32)

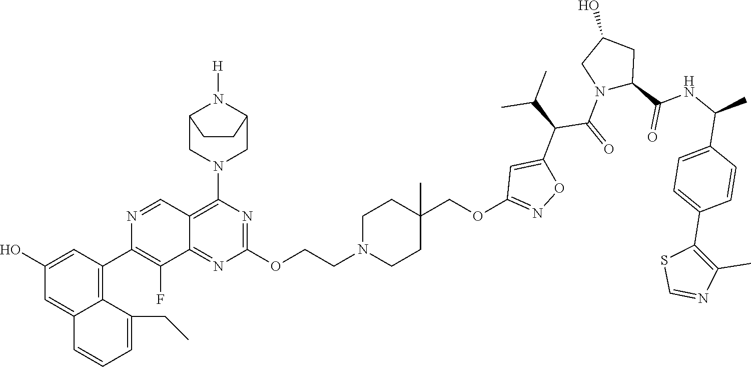 [SMILES: CCc1cccc2cc(O)cc(-c3ncc4c(N5CC6CCC(C5)N6)nc(OCCN5CCC(C)(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

The title compound was prepared in an analogous manner to Compound 31 starting from tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-piperidine-1-carboxylate. (white solid, formate). MS (ESI) m/z: 1178.9 [M+H]⁺; ¹H NMR (400 MHz, CD₃OD) δ 9.09 (s, 1H), 8.88 (s, 1H), 7.64 (d, J=4.0 Hz, 1H), 7.45-7.35 (m, 5H), 7.30-7.29 (m, 1H), 7.17-7.16 (m, 1H), 7.01 (s, 1H), 6.01 (s, 1H), 5.04-5.00 (m, 1H), 4.72-4.67 (m, 4H), 4.52-4.44 (m, 2H), 4.02 (s, 2H), 3.93-3.91 (m, 2H), 3.85-3.77 (m, 3H), 3.69-3.66 (m, 1H), 3.62-3.59 (m, 1H), 3.19-3.13 (m, 2H), 3.07-2.98 (m, 2H), 2.48 (s, 3H), 2.40-2.15 (m, 5H), 2.00-1.85 (m, 8H), 1.64-1.57 (m, 3H), 1.52 (d, J=3.6 Hz, 2H), 1.13 (s, 3H), 1.05 (d, J=3.2 Hz, 3H), 0.91-0.87 (m, 6H).

Exemplary Synthesis of (2S,4R)-1-[2-(3-{[(3R,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 38)

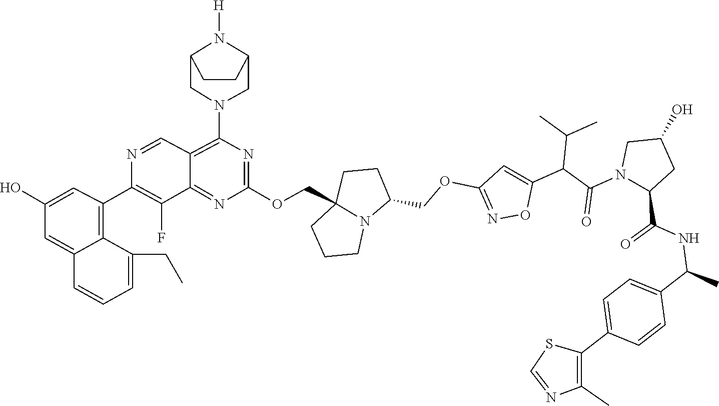 [SMILES: CCc1cccc2cc(O)cc(-c3ncc4c(N5CC6CCC(C5)N6)nc(OC[C@]56CCCN5[C@@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

The title compound was prepared in an analogous manner to Compound 28. (white solid, formate). MS (ESI) m/z: 1079.9 [M+H]⁺; ¹H NMR (400 MHz, CD₃OD) δ 9.09 (s, 1H), 8.90-8.82 (m, 1H), 7.66-7.57 (m, 1H), 7.47-7.24 (m, 6H), 7.18-7.09 (m, 1H), 7.06-6.98 (m, 1H), 6.06-5.97 (m, 1H), 4.75-4.29 (m, 11H), 3.86-3.63 (m, 6H), 3.63-3.34 (m, 3H), 2.52-2.39 (m, 4H), 2.38-2.27 (m, 4H), 2.17-1.84 (m, 11H), 1.54-1.36 (m, 3H), 1.05 (br d, J=5.6 Hz, 3H), 0.92-0.84 (m, 6H).

Exemplary Synthesis of [(3R*,7aR**)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R***)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 107)

Step 1: Preparation of 4-amino-6-chloro-5-fluoronicotinic acid

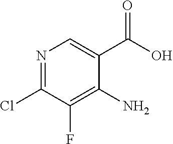 [SMILES: Nc1c(C(=O)O)cnc(Cl)c1F]

To a solution of ethyl 4-amino-6-chloro-5-fluoronicotinate (104 g, 475 mmol, 1 eq) in tetrahydrofuran (900 mL) was added sodium hydroxide (76.11 g, 1.9 mol, 4 eq) and water (450 mL), the mixture was stirred at 25° C. for 16 h, then concentrated. Acidified with 2 M hydrochloric acid to pH 2, the suspension was filtered, the filter cake was triturated with dichloromethane (500 mL) to afford 4-amino-6-chloro-5-fluoronicotinic acid (61 g, 67%) as a gray solid. MS (ESI) m/z: 190.9 [M+1]⁺; 1H NMR (400 MHz, DMSO-d₆) δ 13.79-13.30 (m, 1H), 8.36 (s, 1H), 7.60 (br s, 2H).

Step 2: Preparation of 7-chloro-8-fluoro-2-thioxo-2,3-dihydropyrido[4,3-d]pyrimidin-4 (1H)-one

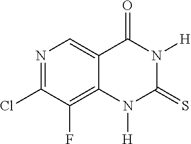 [SMILES: O=c1[nH]c(=S)[nH]c2c(F)c(Cl)ncc12]

To a solution of 4-amino-6-chloro-5-fluoro-pyridine-3-carboxylic acid (31.0 g, 162 mmol) in phosphorus oxychloride (250 mL) was stirred at 90° C. for 2 h. The mixture was concentrated in vacuum to afford 4-amino-6-chloro-5-fluoronicotinoyl chloride (33.5 g, 98%) as a gray solid. This material was dissolved in tetrahydrofuran (300 mL), a solution of ammonium thiocyanate (24.40 g, 320 mmol, 24.4 mL, 2 eq) in tetrahydrofuran (200 mL) was added, the mixture was stirred at 60° C. for 12 h, then filtered and the filtrate was concentrated in vacuum. The residue was triturated with ethyl acetate (300 mL) to afford 7-chloro-8-fluoro-2-thioxo-2,3-dihydropyrido[4,3-d]pyrimidin-4 (1H)-one (23 g, 62%) as a yellow solid. ¹H NMR (400 MHz, DMSO-d₆) δ 13.32 (br s, 1H), 12.90 (br s, 1H), 8.65 (s, 1H)

Step 3: Preparation of 7-chloro-8-fluoro-2-(methylthio)pyrido[4,3-d]pyrimidin-4 (3H)-one

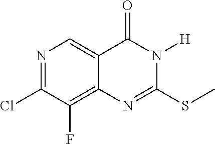

To a solution of 7-chloro-8-fluoro-2-thioxo-2,3-dihydropyrido[4,3-d]pyrimidin-4 (1H)-one (45 g, 194.27 mmol, 1 eq) in N,N-dimethylformamide (450 mL) was added sodium methoxide (10.50 g, 194.27 mmol, 1 eq), the mixture was stirred at 25° C. for 10 min, then iodomethane (27.57 g, 194.27 mmol, 12.1 mL, 1 eq) was added, the mixture was stirred at 25° C. for 50 min. Then the mixture was poured into ice water (2000 mL), filtered, washed with water (300 mL×3) to afford 7-chloro-8-fluoro-2-(methylthio)pyrido[4,3-d]pyrimidin-4 (3H)-one (44 g, 92%) as a yellow solid. MS (ESI) m/z: 246.0 [M+1]⁺; 1H NMR (400 MHz, DMSO-d₆) δ 13.26 (br s, 1H), 8.81 (s, 1H), 2.61 (s, 3H)

Step 4: Preparation of 4,7-dichloro-8-fluoro-2-(methylthio)pyrido[4,3-d]pyrimidine

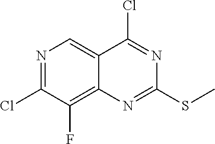

To a solution of 7-chloro-8-fluoro-2-(methylthio)pyrido[4,3-d]pyrimidin-4 (3H)-one (44.0 g, 179 mmol, 1 eq) in phosphorus oxychloride (440 mL) was added N,N-diisopropylethylamine (46.30 g, 358 mmol, 62.4 mL), the mixture was stirred at 90° C. for 30 min, then concentrated in vacuum to afford 4,7-dichloro-8-fluoro-2-(methylthio)pyrido[4,3-d]pyrimidine (47 g, 99%) as a brown oil.

Step 5: Preparation of ((1R,5S)-tert-butyl 3-(7-chloro-8-fluoro-2-(methylthio)pyrido[4,3-d]pyrimidin-4-yl)-3,8-diazabicyclo[3.2.1]octane-8-carboxylate

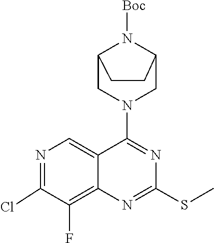 [SMILES: CSc1nc(N2CC3CCC(C2)N3C(=O)OC(C)(C)C)c2cnc(Cl)c(F)c2n1]

To a solution of 4,7-dichloro-8-fluoro-2-(methylthio)pyrido[4,3-d]pyrimidine (47.0 g, 178 mmol, 1 eq) in dichloromethane (470 mL) was added tert-butyl 3,8-diazabicyclo[3.2.1]octane-8-carboxylate (56.67 g, 267 mmol, 1.5 eq) and N,N-diisopropylethylamine (115.00 g, 890 mmol, 155.0 mL), the mixture was stirred at −40° C. for 1 h, diluted with water (300 mL) and extracted with dichloromethane (300 mL×2). The combined organic layers were washed with brine (300 mL×2), dried over anhydrous sodium sulfate, filtered, and concentrated. The residue was purified by silica gel chromatography (petroleum ether/ethyl acetate=1/0 to 0/1), then further triturated with acetonitrile (500 mL) to afford ((1R,5S)-tert-butyl 3-(7-chloro-8-fluoro-2-(methylthio)pyrido[4,3-d]pyrimidin-4-yl)-3,8-diazabicyclo[3.2.1]octane-8-carboxylate (43.8 g, 53%) as an off-white solid. MS (ESI) m/z: 440.2 [M/2+1]⁺; 1H NMR (400 MHz, DMSO-d₆) δ 8.92 (s, 1H), 4.49 (br d, J=12.4 Hz, 2H), 4.24 (br s, 2H), 3.63 (br d, J=12.4 Hz, 2H), 2.55 (s, 3H), 1.86-1.73 (m, 2H), 1.60 (br d, J=7.6 Hz, 2H), 1.46 (s, 9H).

Step 6: Preparation of tert-butyl 3-[7-[8-ethyl-3-(methoxymethoxy)-1-naphthyl]-8-fluoro-2-methylsulfanyl-pyrido[4,3-d]pyrimidin-4-yl]-3,8-diazabicyclo[3.2.1]octane-8-carboxylate

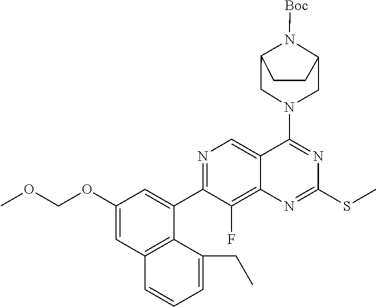

A mixture of tert-butyl 3-(7-chloro-8-fluoro-2-methylsulfanyl-pyrido[4,3-d]pyrimidin-4-yl)-3,8-diazabicyclo[3.2.1]octane-8-carboxylate (3 g, 7 mmol, 1 eq), 2-[8-ethyl-3-(methoxymethoxy)-1-naphthyl]-4,4,5,5-tetramethyl-1,3,2-dioxaborolane (3.03 g, 9 mmol, 1.3 eq), potassiumphosphate (4.34 g, 20 mmol, 3 eq) and mesylate[(di(1-adamantyl)-n-butylphosphine)-2-(2′-amino-1,1′-biphenyl)]palladium (II) (497 mg, 0.7 mmol, 0.1 eq) in dioxane (45 mL) and water (4.5 mL) was de-gassed and then heated to 80° C. for 12 h under nitrogen. The mixture was cooled to 25° C., diluted with ethyl acetate (300 mL) and washed with brine (100 mL). The organic layer was dried over anhydrous sodium sulfate, filtered, and concentrated under vacuum. The residue was purified by silica gel column chromatography (petroleum ether/ethyl acetate=10/1 to 4:1) to afford tert-butyl 3-[7-[8-ethyl-3-(methoxymethoxy)-1-naphthyl]-8-fluoro-2-methylsulfanyl-pyrido[4,3-d]pyrimidin-4-yl]-3,8-diazabicyclo[3.2.1]octane-8-carboxylate (3.8 g, 90%) as a light brown solid. MS (ESI) m/z: 620.4 [M+1]⁺; ¹H NMR (400 MHz, DMSO-d₆) δ 9.14 (s, 1H), 7.80 (d, J=8.0 Hz, 1H), 7.62 (d, J=2.8 Hz, 1H), 7.45 (t, J=7.6 Hz, 1H), 7.24 (d, J=7.2 Hz, 1H), 7.17 (d, J=2.8 Hz, 1H), 5.35 (s, 2H), 4.57 (br s, 1H), 4.49 (br s, 1H), 4.29 (br d, J=16.0 Hz, 2H), 3.72 (br d, J=12.4 Hz, 1H), 3.63 (br d, J=12.8 Hz, 1H), 3.43 (s, 3H), 2.56 (s, 3H), 2.39-2.19 (m, 2H), 1.84 (br d, J=2.4 Hz, 2H), 1.74-1.63 (m, 2H), 1.46 (s, 9H), 0.83 (t, J=7.6 Hz, 3H).

Step 7: Preparation of tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate

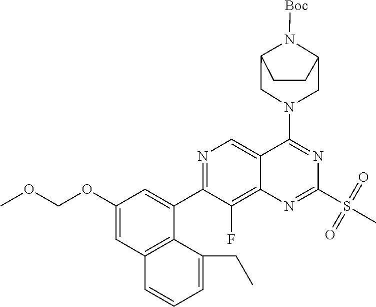

To a solution of tert-butyl 3-[7-[8-ethyl-3-(methoxymethoxy)-1-naphthyl]-8-fluoro-2-methylsulfanyl-pyrido[4,3-d]pyrimidin-4-yl]-3,8-diazabicyclo[3.2.1]octane-8-carboxylate (2 g, 3 mmol, 1 eq) in N,N-dimethylformamide (20 mL) was added 4 A MS (2 g), the mixture was stirred at 25° C. for 3 h. To the mixture was added oxone (5.95 g, 10 mmol, 3 eq), the mixture was stirred at 25° C. for 12 h. The mixture was filtered with a pad of Celite and washed with ethyl acetate (400 mL). The organic layer was washed with brine (60 mL×3), dried over anhydrous sodium sulfate, filtered, and concentrated. The residue was purified by silica gel column chromatography (petroleum ether/ethyl acetate=1/0 to 2:1) to afford tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (2 g, 95%) as a yellow gum. MS (ESI) m/z: 652.2 [M+1]⁺; 1H NMR (400 MHz, CDCl₃) δ 9.19 (s, 1H), 7.73 (d, J=7.6 Hz, 1H), 7.57 (d, J=2.8 Hz, 1H), 7.44 (t, J=7.6 Hz, 1H), 7.26 (br s, 1H), 7.19 (d, J=2.4 Hz, 1H), 5.32 (d, J=2.0 Hz, 2H), 4.91-4.56 (m, 2H), 4.45 (br d, J=2.8 Hz, 2H), 3.97-3.65 (m, 2H), 3.53 (s, 3H), 3.44 (s, 3H), 2.29 (t, J=7.2 Hz, 2H), 2.02 (br dd, J=2.0, 7.6 Hz, 1H), 1.82-1.66 (m, 3H), 1.53 (s, 9H), 0.94 (t, J=7.6 Hz, 3H).

Step 8: Preparation of tert-butyl 3-[2-[[(3S,8S)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

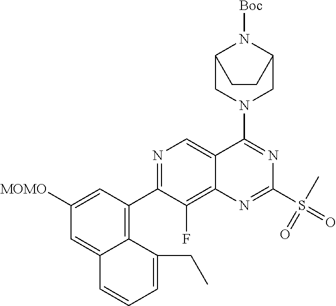

To a solution of tert-butyl 3-[7-[8-ethyl-3-(methoxymethoxy)-1-naphthyl]-8-fluoro-2-methyl sulfonyl-pyrido[4,3-d]pyrimidin-4-yl]-3,8-diazabicyclo[3.2.1]octane-8-carboxylate (500 mg, 0.8 mmol, 1 eq) and [(3S,8S)-3-[[tert-butyl(diphenyl)silyl]oxymethyl]-1,2,3,5,6,7-hexahydropyrrolizin-8-yl]methanol (346 mg, 0.8 mmol, 1.1 eq) in tetrahydrofuran (8 mL) was added lithium tert-butoxide (2.2 M, 1.1 mL, 3 eq) at 0° C., the mixture was stirred at 25° C. for 1 h, then diluted with ethyl acetate (200 mL). The organic layer was washed with saturated ammonium chloride solution (20 mL×3). The organic layer was dried over anhydrous sodium sulfate, filtered, and concentrated. The residue was purified by silica gel column chromatography (petroleum ether/ethyl acetate=1/0 to 1:1) to afford tert-butyl 3-[2-[[(3S,8S)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (340 mg, 45%) as a colorless gum. MS (ESI) m/z: 981.2 [M+1]⁺.

Step 9: Preparation of [(3R*,7aR**)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R***)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate

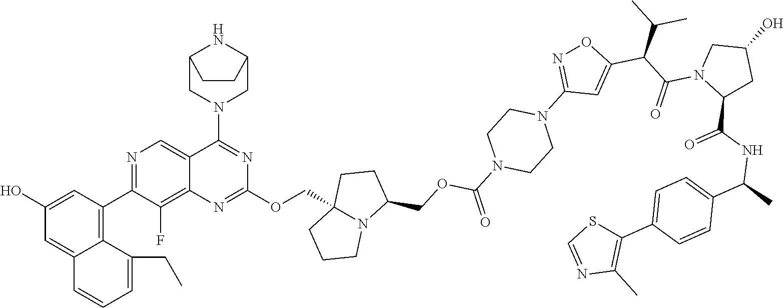 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 7 starting from tert-butyl 3-[2-[[(3R,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate. (formate, white solid). MS (ESI) m/z: 1192.4 [M+1]⁺; 1H NMR (400 MHz, MeOD-d₄) δ 9.10 (s, 1H), 8.87 (s, 1H), 8.49 (s, 1H), 7.62 (br d, J=8.0 Hz, 1H), 7.48-7.32 (m, 5H), 7.28 (d, J=2.0 Hz, 1H), 7.16 (br d, J=7.2 Hz, 1H), 7.00 (d, J=2.8 Hz, 1H), 6.18-6.02 (m, 1H), 5.03 (br d, J=7.2 Hz, 1H), 4.69-4.61 (m, 2H), 4.57-4.27 (m, 5H), 4.17-4.04 (m, 1H), 3.88-3.70 (m, 5H), 3.67-3.42 (m, 7H), 3.21 (br s, 4H), 2.50-2.24 (m, 8H), 2.21-2.12 (m, 3H), 2.11-1.81 (m, 10H), 1.51 (d, J=7.2 Hz, 3H), 1.04 (br d, J=6.8 Hz, 3H), 0.93-0.84 (m, 6H).

Exemplary Synthesis of [(3R,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R*)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 108)

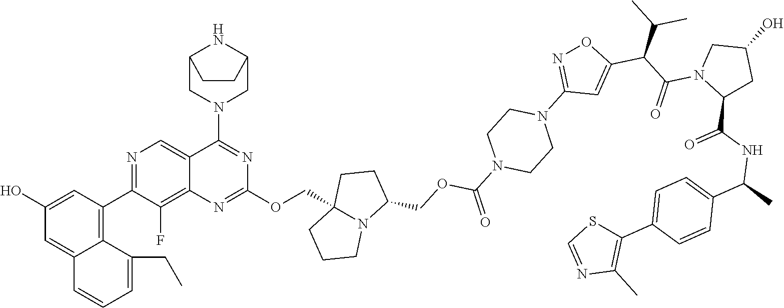 [SMILES: CCc1cccc2cc(O)cc(-c3ncc4c(N5CC6CCC(C5)N6)nc(OC[C@@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 107 starting from [(3R,8S)-3-[[tert-butyl(diphenyl)silyl]oxymethyl]-1,2,3,5,6,7-hexahydropyrrolizin-8-yl]methanol. (formate, white solid. MS (ESI) m/z: 1191.6 [M+1]⁺; 1H NMR (400 MHz, MeOD-d₄) δ 9.10 (s, 1H), 8.91-8.86 (m, 1H), 8.48 (s, 1H), 7.63 (dd, J=3.2, 8.4 Hz, 1H), 7.49-7.32 (m, 5H), 7.29 (dd, J=2.8, 5.2 Hz, 1H), 7.17 (d, J=6.8 Hz, 1H), 7.03 (dd, J=2.8, 4.8 Hz, 1H), 6.09 (d, J=10.4 Hz, 1H), 5.06-5.01 (m, 1H), 4.80-4.69 (m, 2H), 4.55-4.48 (m, 1H), 4.46-4.38 (m, 3H), 4.33-4.23 (m, 1H), 4.18-4.08 (m, 1H), 4.03-3.92 (m, 2H), 3.88-3.73 (m, 3H), 3.68-3.45 (m, 7H), 3.23-3.01 (m, 5H), 2.53-2.44 (m, 3H), 2.41-2.26 (m, 4H), 2.21-1.78 (m, 14H), 1.65-1.47 (m, 3H), 1.10-0.99 (m, 3H), 0.96-0.78 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R*)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 109)

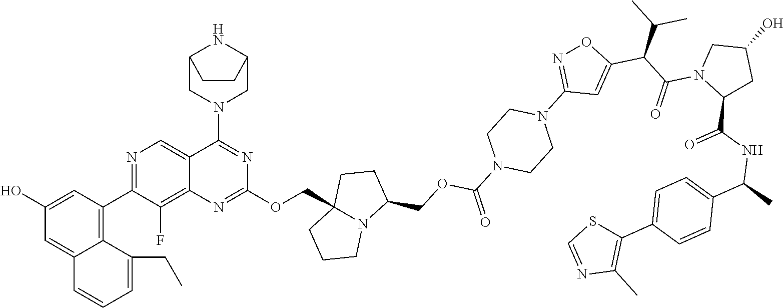 [SMILES: CCc1cccc2cc(O)cc(-c3ncc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 107 starting from [(3S,8R)-3-[[tert-butyl(diphenyl)silyl]oxymethyl]-1,2,3,5,6,7-hexahydropyrrolizin-8-yl]methanol. (formate, white solid). MS (ESI) m/z: 1191.6 [M+1]⁺; 1H NMR (400 MHz, MeOD-d₄) δ 9.18-9.06 (m, 1H), 8.91-8.84 (m, 1H), 8.46 (s, 1H), 7.63 (d, J=8.0 Hz, 1H), 7.47-7.33 (m, 5H), 7.30-7.24 (m, 1H), 7.21-7.12 (m, 1H), 7.03 (dd, J=2.8, 6.8 Hz, 1H), 6.08 (d, J=6.0 Hz, 1H), 5.03 (q, J=7.2 Hz, 1H), 4.80-4.68 (m, 2H), 4.55-4.38 (m, 4H), 4.35-4.24 (m, 1H), 4.21-4.11 (m, 1H), 3.98 (br d, J=8.4 Hz, 2H), 3.91-3.73 (m, 3H), 3.69-3.48 (m, 6H), 3.45-3.38 (m, 1H), 3.22-2.98 (m, 5H), 2.50-2.43 (m, 3H), 2.43-2.24 (m, 4H), 2.23-1.79 (m, 14H), 1.61-1.44 (m, 3H), 1.11-1.01 (m, 3H), 0.95-0.83 (m, 6H).

Exemplary Synthesis of [(3R*,7aR**)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-{5-[(2R***)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 110)

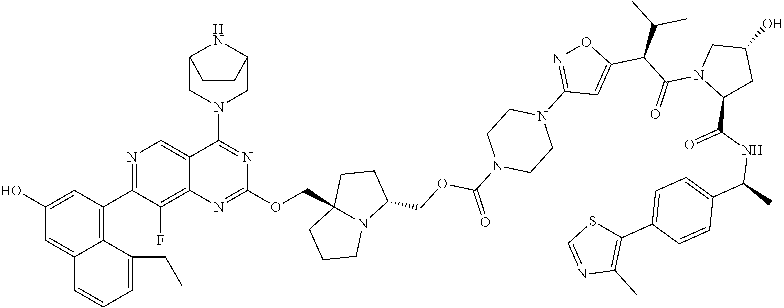 [SMILES: CCc1cccc2cc(O)cc(-c3ncc4c(N5CC6CCC(C5)N6)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to Compound 107 starting from [(3R,8R)-3-[[tert-butyl(diphenyl)silyl]oxymethyl]-1,2,3,5,6,7-hexahydropyrrolizin-8-yl]methanol. (formate, white solid). MS (ESI) m/z: 1191.5 [M+1]⁺; 1H NMR (400 MHz, MeOD-d₄) δ 9.13 (s, 1H), 8.88 (s, 1H), 8.43 (br s, 2H), 7.63 (d, J=8.0 Hz, 1H), 7.49-7.33 (m, 5H), 7.30 (t, J=2.4 Hz, 1H), 7.17 (d, J=6.8 Hz, 1H), 7.06-6.96 (m, 1H), 6.13 (d, J=1.2 Hz, 1H), 5.03 (br d, J=6.8 Hz, 1H), 4.76-4.68 (m, 2H), 4.65-4.47 (m, 3H), 4.45-4.34 (m, 2H), 4.30-4.20 (m, 1H), 4.10-4.00 (m, 2H), 3.98-3.89 (m, 1H), 3.88-3.74 (m, 2H), 3.68-3.50 (m, 7H), 3.38 (br d, J=5.6 Hz, 1H), 3.22 (br s, 4H), 2.51-2.45 (m, 3H), 2.43-2.30 (m, 4H), 2.29-2.16 (m, 4H), 2.16-1.90 (m, 10H), 1.60-1.48 (m, 3H), 1.05 (br d, J=6.4 Hz, 3H), 0.93-0.80 (m, 6H).

Exemplary Synthesis of (2S,4R)-1-[(2R)-2-{3-[(1-{[(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 111)

Step 1: Preparation of tert-butyl-3-[7-[8-ethyl-3-(methoxymethoxy)-1-naphthyl]-8-fluoro-2-[[(3S,8R)-3-formyl-1,2,3,5,6,7-hexahydropyrrolizin-8-yl]methoxy]pyrido[4,3-d]pyrimidin-4-yl]-3,8-diazabicyclo[3.2.1]octane-8-carboxylate

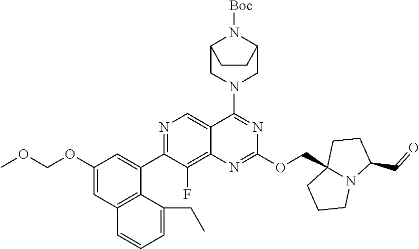 [SMILES: CCc1cccc2cc(OCOC)cc(-c3ncc4c(N5CC6CCC(C5)N6C(=O)OC(C)(C)C)nc(OC[C@]56CCCN5[C@H](C=O)CC6)nc4c3F)c12]

To a solution of tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (450 mg, 0.6 mmol) in THF (12 mL) were added NMO (177 mg, 1.5 mmol) and tetrapropylammonium perruthenate (43 mg, 0.1 mmol). The mixture was stirred at 20° C. for 3 h. The mixture was diluted with CH₂Cl₂ (20 mL), filtered through Celite pad, and rinsed with CH₂Cl₂ (10 mL×3). The combined organic phase was washed with water (9 mL×2) and brine (10 mL), dried over anhydrous Na₂SO₄, filtered, and concentrated to afford tert-butyl 3-[7-[8-ethyl-3-(methoxymethoxy)-1-naphthyl]-8-fluoro-2-[[(3S,8R)-3-formyl-1,2,3,5,6,7-hexahydropyrrolizin-8-yl]methoxy]pyrido[4,3-d]pyrimidin-4-yl]-3,8-diazabicyclo[3.2.1]octane-8-carboxylate (448 mg) in CH₂Cl₂ (4.0 mL), which was used directly in the next step. MS (ESI) m/z: 759.5 [M+H₂O+1]⁺.

Step 2: Preparation of tert-butyl-3-[7-[8-ethyl-3-(methoxymethoxy)-1-naphthyl]-8-fluoro-2-[[(3S,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

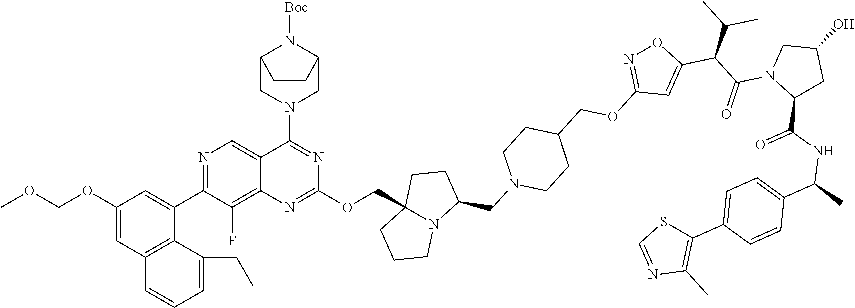 [SMILES: CCc1cccc2cc(OCOC)cc(-c3ncc4c(N5CC6CCC(C5)N6C(=O)OC(C)(C)C)nc(OC[C@]56CCCN5[C@H](CN5CCC(COc7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-[8-ethyl-3-(methoxymethoxy)-1-naphthyl]-8-fluoro-2-[[(3S,8R)-3-formyl-1,2,3,5,6,7-hexahydropyrrolizin-8-yl]methoxy]pyrido[4,3-d]pyrimidin-4-yl]-3,8-diazabicyclo[3.2.1]octane-8-carboxylate (512 mg, 70% purity, 0.48 mmol, 1.0 eq) in anhydrous CH₂Cl₂ (12 mL) was added (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide (288 mg, 0.48 mmol, 1.0 eq) and NaBH(OAc)₃ (256 mg, 1.2 mmol, 2.5 eq). The mixture was stirred at 25° C. for 16 h, quenched by saturated NaHCO₃ solution (15 mL) at 20° C. and extracted with dichloromethane (40 mL×3). The combined organic phase was washed with brine (30 mL), dried over anhydrous Na₂SO₄, filtered, and concentrated. The residue was purified by flash column chromatography (0˜10% methanol in dichloromethane) to afford tert-butyl-3-[7-[8-ethyl-3-(methoxymethoxy)-1-naphthyl]-8-fluoro-2-[[(3S,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (175 mg, 21%) as black brown solid. MS (ESI) m/z: 661.1 [M/2+1]⁺.

Step 3: Preparation of (2S,4R)-1-[(2R)-2-{3-[(1-{[(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

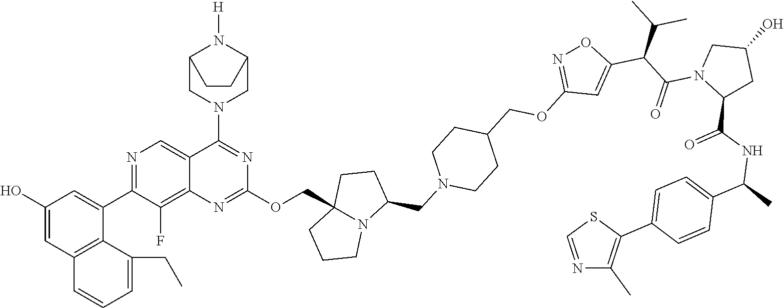 [SMILES: CCc1cccc2cc(O)cc(-c3ncc4c(N5CC6CCC(C5)N6)nc(OC[C@]56CCCN5[C@H](CN5CCC(COc7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

To a solution of tert-butyl 3-[7-[8-ethyl-3-(methoxymethoxy)-1-naphthyl]-8-fluoro-2-[[(3S,8R)-3-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-1-piperidyl]methyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (250 mg, 0.2 mmol, 1.0 eq) in CH₂Cl₂ (4.0 mL) was added 4 M HCl/dioxane (4.0 mL). The mixture was stirred at 20° C. for 30 min. The mixture was suspended in petroleum ether (50 mL) and filtered. The filter cake was re-dissolved in THF (40 mL) and Et₃N (3.0 mL), then filtered. The filtrate was concentrated to dryness and the residue was purified by preparative HPLC (column: Phenomenex C18 75*30 mm*3 um; mobile phase: [water (FA)-MeCN]; B %: 6-46%; 28 min) to afford (2S,4R)-1-[(2R)-2-{3-[(1-{[(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl}piperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (formate, 107.5 mg, 47%) as a yellow solid. MS (ESI) m/z: 1177.0 [M+1]⁺; 1H NMR (400 MHz, CD₃OD) δ 9.18-9.07 (m, 1H), 8.91-8.84 (m, 1H), 8.51-8.41 (m, 2H), 7.68-7.59 (m, 1H), 7.49-7.27 (m, 6H), 7.19-7.13 (m, 1H), 7.07-6.97 (m, 1H), 6.03-5.80 (m, 1H), 5.07-5.01 (m, 1H), 4.78-4.67 (m, 2H), 4.63-4.47 (m, 2H), 4.46-4.36 (m, 2H), 4.11-3.88 (m, 4H), 3.88-3.74 (m, 2H), 3.73-3.41 (m, 6H), 3.22-2.99 (m, 2H), 2.94-2.81 (m, 1H), 2.79-2.68 (m, 1H), 2.66-2.52 (m, 1H), 2.49-2.44 (m, 3H), 2.40-2.20 (m, 6H), 2.11-1.78 (m, 15H), 1.59-1.34 (m, 5H), 1.11-1.00 (m, 3H), 0.95-0.80 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 6-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2,6-diazaspiro[3.3]heptane-2-carboxylate (Compound 112)

Step 1: Preparation of tert-butyl 6-[5-(1-methoxycarbonyl-2-methyl-propyl)isoxazol-3-yl]-2,6-diazaspiro[3.3]heptane-2-carboxylate

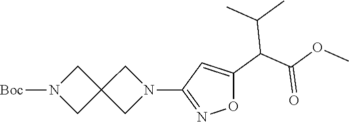 [SMILES: COC(=O)C(c1cc(N2CC3(CN(C(=O)OC(C)(C)C)C3)C2)no1)C(C)C]

To a mixture of tert-butyl 2,6-diazaspiro[3.3]heptane-2-carboxylate (1.5 g as semi oxalic acid salt, 3.1 mmol, 0.50 eq) and 4 A MS (3.0 g) in N,N-dimethylacetamide (30 mL) was added triethylamine (2.6 mL, 19 mmol, 3.0 eq) and methyl 3-methyl-2-[3-(1,1,2,2,3,3,4,4,4-nonafluorobutylsulfonyloxy)isoxazol-5-yl]butanoate (3.0 g, 6.2 mmol, 1.0 eq). The mixture was stirred at 130° C. for 16 h, then diluted with ethyl acetate (40 mL), and washed with water (30 mL×5). The combined organic phase was dried over anhydrous Na₂SO₄, filtered, and concentrated. The residue was purified by column chromatography (0˜26% ethyl acetate in petroleum ether) to afford tert-butyl 6-[5-(1-methoxycarbonyl-2-methyl-propyl)isoxazol-3-yl]-2,6-diazaspiro[3.3]heptane-2-carboxylate (951 mg, 39%) as light yellow oil. MS (ESI) m/z: 380.3 [M+H]⁺.

Step 2: Preparation of 2-[3-(2-tert-butoxycarbonyl-2,6-diazaspiro[3.3]heptan-6-yl)isoxazol-5-yl]-3-methyl-butanoic acid

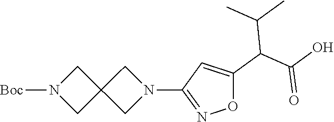 [SMILES: CC(C)C(C(=O)O)c1cc(N2CC3(CN(C(=O)OC(C)(C)C)C3)C2)no1]

To a stirred solution of tert-butyl 6-[5-(1-methoxycarbonyl-2-methyl-propyl)isoxazol-3-yl]-2,6-diazaspiro[3.3]heptane-2-carboxylate (951 mg, 2.5 mmol, 1.0 eq) in tetrahydrofuran (10 mL) and water (5 mL) was added lithium hydroxide hydrate (420 mg, 10 mmol, 4.0 eq). The mixture was stirred at 30° C. for 16 h, then concentrated, the pH of the residue was adjusted to 3-4 by hydrochloric acid (2 M). The mixture was extracted with dichloromethane (15 mL×3). The combined organic extracts were washed with brine (15 mL), dried over anhydrous Na₂SO₄, filtered, and concentrated under reduced pressure to afford 2-[3-(2-tert-butoxycarbonyl-2,6-diazaspiro[3.3]heptan-6-yl)isoxazol-5-yl]-3-methyl-butanoic acid (810 mg, crude) as a white solid. MS (ESI) m/z: 366.3 [M+H]⁺.

Step 3: Preparation of tert-butyl 6-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate

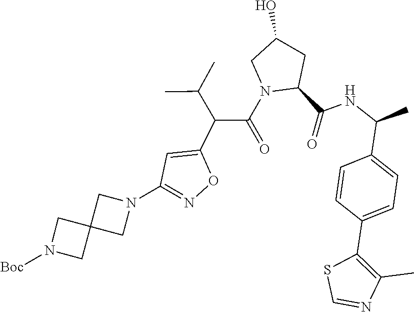 [SMILES: Cc1ncsc1-c1ccc([C@H](C)NC(=O)[C@@H]2C[C@@H](O)CN2C(=O)C(c2cc(N3CC4(CN(C(=O)OC(C)(C)C)C4)C3)no2)C(C)C)cc1]

To a solution of 2-[3-[(3R)-4-tert-butoxycarbonyl-3-methyl-piperazin-1-yl]isoxazol-5-yl]-3-methyl-butanoic acid (810 mg, 2.2 mmol, 1.0 eq) and (2S,4R)-4-hydroxy-N-[(1S)-1-[4-(4-methylthiazol-5-yl)phenyl]ethyl]pyrrolidine-2-carboxamide (1.3 g, 2.4 mmol, 1.1 eq) in dichloromethane (15 mL) were added DIEA (1.9 mL, 11 mmol, 5.0 eq) and HATU (927 mg, 2.4 mmol, 1.1 eq). The mixture was stirred at 25° C. for 2 h, then diluted with dichloromethane (10 mL), washed with water (20 mL), saturated sodium bicarbonate solution (20 mL) and brine (20 mL), dried over anhydrous Na₂SO₄, filtered, and concentrated under reduced pressure. The residue was purified by flash column chromatography (0˜100% ethyl acetate in petroleum ether) to afford tert-butyl 6-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate (1.5 g, 93%) as a white solid. MS (ESI) m/z: 679.4 [M+H]⁺.

Step 4: Preparation of tert-butyl 6-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate and tert-butyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate

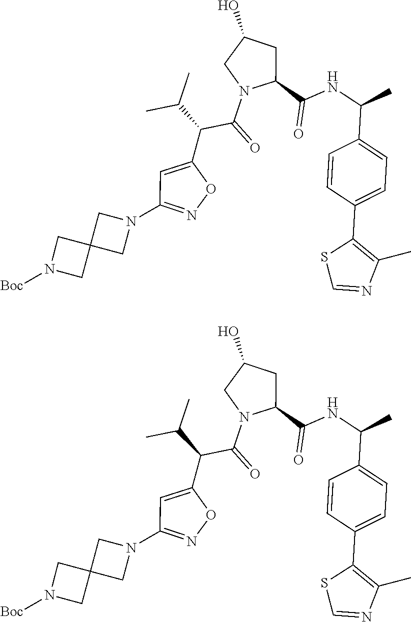 [SMILES: Cc1ncsc1-c1ccc([C@H](C)NC(=O)[C@@H]2C[C@@H](O)CN2C(=O)[C@H](c2cc(N3CC4(CN(C(=O)OC(C)(C)C)C4)C3)no2)C(C)C)cc1 ; Cc1ncsc1-c1ccc([C@H](C)NC(=O)[C@@H]2C[C@@H](O)CN2C(=O)[C@@H](c2cc(N3CC4(CN(C(=O)OC(C)(C)C)C4)C3)no2)C(C)C)cc1]

A solution of tert-butyl 6-[5-[1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate (1.6 g) in methanol (10 mL) was separated by chiral SFC {DAICEL CHIRALCEL OD (250 mm*50 mm, 10 um); mobile phase: [0.1% NH₃H₂O IPA]; B %: 45%}.

Tert-butyl 6-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate (840 mg) was obtained as light yellow foam. MS (ESI) m/z: 679.2 [M+H]⁺.

Tert-butyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate (712 mg) was obtained as light yellow foam. MS (ESI) m/z: 679.3 [M+H]⁺.

Step 5: Preparation of (2S,4R)-1-[(2R)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide

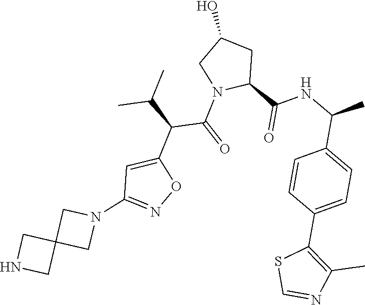 [SMILES: Cc1ncsc1-c1ccc([C@H](C)NC(=O)[C@@H]2C[C@@H](O)CN2C(=O)[C@@H](c2cc(N3CC4(CNC4)C3)no2)C(C)C)cc1]

To a stirred solution of tert-butyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate (300 mg, 0.3 mmol) in dichloromethane (3.0 mL) was added trifluoroacetic acid (1.0 mL, 13.3 mmol). The reaction was stirred at 20° C. for 30 min, then concentrated to afford (2S,4R)-1-[(2R)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide trifluoroacetate (280 mg) as light yellow solid. MS (ESI) m/z: 579.5 [M+H]⁺.

Step 6: Preparation of tert-butyl 3-[7-[8-ethyl-3-(methoxymethoxy)-1-naphthyl]-8-fluoro-2-[[(3S,8S)-3-[[6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

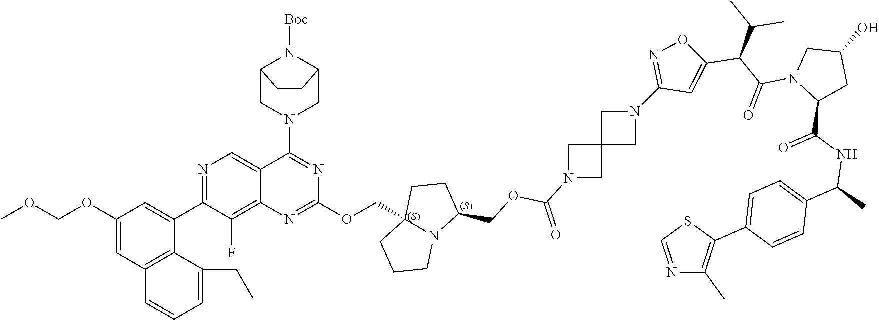

To a mixture of tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (150 mg, 0.2 mmol, 1.0 eq) in THF (10 mL) was added triethylamine (0.141 mL, 1.0 mmol, 5.01 eq), DMAP (7.4 mg, 0.05 mmol, 0.30 eq) and (4-nitrophenyl) chloroformate (77.3 mg, 0.4 mmol, 1.9 eq), the resulting suspension was stirred at 30° C. for 15 h. Then (2S,4R)-1-[(2R)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide trifluoroacetate (193.9 mg, 0.3 mmol, 1.39 eq) was added, the resulting suspension was stirred at 30° C. for 2 h. The reaction mixture was filtered and concentrated under reduced pressure. The residue was purified by flash column chromatography (0-6% to 8% MeOH in CH₂Cl₂) to afford tert-butyl 3-[7-[8-ethyl-3-(methoxymethoxy)-1-naphthyl]-8-fluoro-2-[[(3S,8S)-3-[[6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (271 mg, 90% purity, 90%) as a white solid. MS (ESI) m/z: 1347.7 [M+H]⁺.

Step 7: Preparation of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 6-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2,6-diazaspiro[3.3]heptane-2-carboxylate

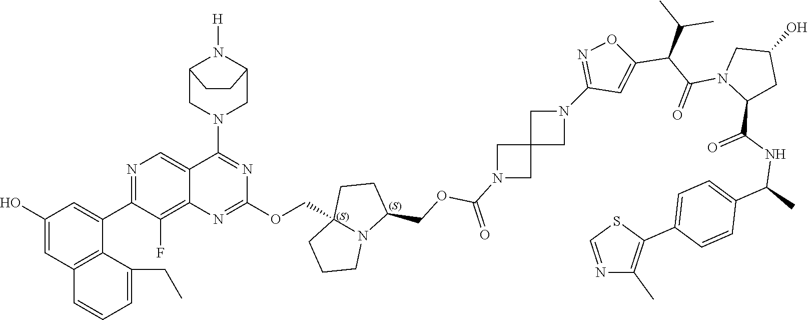

To a mixture of tert-butyl 3-[7-[8-ethyl-3-(methoxymethoxy)-1-naphthyl]-8-fluoro-2-[[(3S,8S)-3-[[6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (271 mg, 0.2 mmol, 1.0 eq) in CH₂Cl₂ (4.5 mL) was added TFA (1.5 mL, 20 mmol), the resulting suspension was stirred at 20° C. for 30 min. Nitrogen was allowed to bubble through the reaction to remove volatiles. The residue was quenched by saturated NaHCO₃ solution until pH 8 and extracted with CH₂Cl₂/MeOH (v/v=10/1, 20 mL×3). The combined organic layer was dried over Na₂SO₄, filtered, and concentrated under reduced pressure. The crude product was purified by preparative HPLC (column: Phenomenex C18 75*30 mm*3 um; mobile phase: [water (FA)-MeCN]; B %: 0˜40; gradient Time: 26 min; 25 mL/min) to afford [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 6-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2,6-diazaspiro[3.3]heptane-2-carboxylate (formate, 100.5 mg, 43%) as a white solid. MS (ESI) m/z: 1203.9 [M+H]⁺; ¹H NMR (400 MHz, DMSO-d₆) δ 9.09 (s, 1H), 9.02-8.95 (m, 1H), 8.41 (d, J=7.6 Hz, 1H), 7.66 (d, J=8.0 Hz, 1H), 7.48-7.40 (m, 2H), 7.39-7.32 (m, 3H), 7.28 (d, J=2.4 Hz, 1H), 7.12 (d, J=7.2 Hz, 1H), 6.97 (d, J=2.4 Hz, 1H), 5.85 (s, 1H), 4.90 (quint, J=7.2 Hz, 1H), 4.46 (t, J=15.6 Hz, 2H), 4.34 (t, J=7.8 Hz, 1H), 4.27 (s, 1H), 4.23-4.14 (m, 2H), 4.13-4.01 (m, 6H), 4.01-3.94 (m, 4H), 3.75-3.59 (m, 12H), 2.81-2.69 (m, 2H), 2.45 (s, 3H), 2.30-2.13 (m, 3H), 2.09-1.96 (m, 2H), 1.81-1.63 (m, 10H), 1.57-1.47 (m, 1H), 1.46-1.34 (m, 3H), 0.99-0.89 (m, 3H), 0.88-0.70 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 6-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2,6-diazaspiro[3.3]heptane-2-carboxylate (Compound 113)

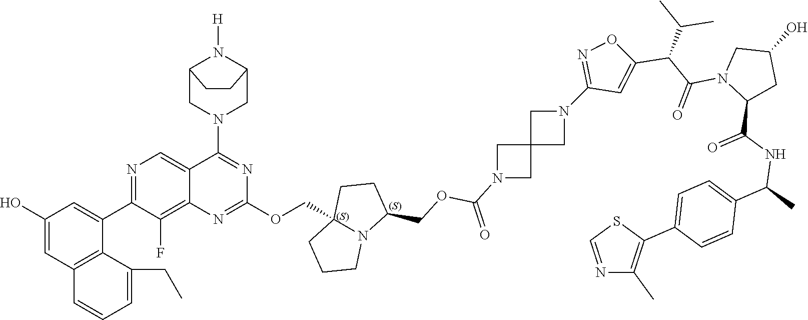

The title compound was prepared in an analogous manner to Compound 112 starting from (2S,4R)-1-[(2S)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formate, white solid). MS (ESI) m/z: 1203.9 [M+H]⁺; ¹H NMR (400 MHz, DMSO-d₆) δ 9.08 (s, 1H), 9.00-8.92 (m, 1H), 8.27 (d, J=7.9 Hz, 1H), 7.66 (d, J=8.4 Hz, 1H), 7.53-7.21 (m, 6H), 7.11 (d, J=7.2 Hz, 1H), 6.96 (d, J=2.4 Hz, 1H), 5.91-5.85 (m, 1H), 5.02-4.82 (m, 1H), 4.53-4.37 (m, 3H), 4.25 (d, J=3.6 Hz, 1H), 4.22-4.12 (m, 2H), 4.11-3.90 (m, 10H), 3.73-3.53 (m, 12H), 2.82-2.69 (m, 2H), 2.46-2.42 (m, 3H), 2.29-2.14 (m, 3H), 2.04 (td, J=3.6, 7.6 Hz, 2H), 1.80-1.62 (m, 10H), 1.57-1.48 (m, 1H), 1.47-1.31 (m, 3H), 0.97-0.68 (m, 9H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-[({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)methyl]azetidine-1-carboxylate (Compound 114)

Step 1: Preparation of tert-butyl 3-[[5-(1-methoxycarbonyl-2-methyl-propyl)isoxazol-3-yl]oxymethyl]azetidine-1-carboxylate

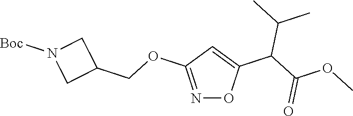 [SMILES: COC(=O)C(c1cc(OCC2CN(C(=O)OC(C)(C)C)C2)no1)C(C)C]

To a solution of methyl 2-(3-hydroxyisoxazol-5-yl)-3-methyl-butanoate (1.0 g, 5 mmol, 1.0 eq) in MeCN (15 mL) were added tert-butyl 3-(bromomethyl)azetidine-1-carboxylate (1.2 g, 5 mmol, 1.0 eq) and K₂CO₃ (1.4 g, 10 mmol, 2.0 eq). The mixture was stirred at 60° C. for 10 h, then filtered and concentrated under reduced pressure. The residue was purified by flash column chromatography (0˜15% ethyl acetate in petroleum ether) to afford tert-butyl 3-[[5-(1-methoxycarbonyl-2-methyl-propyl)isoxazol-3-yl]oxymethyl]azetidine-1-carboxylate (1.37 g, 74%) as colorless oil. MS (ESI) m/z: 369.0 [M+1]⁺.

Step 2: Preparation of 2-[3-[(1-tert-butoxycarbonylazetidin-3-yl)methoxy]isoxazol-5-yl]-3-methyl-butanoic acid

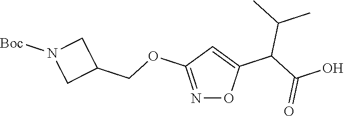 [SMILES: CC(C)C(C(=O)O)c1cc(OCC2CN(C(=O)OC(C)(C)C)C2)no1]

To a solution of tert-butyl 3-[[5-(1-methoxycarbonyl-2-methyl-propyl)isoxazol-3-yl]oxymethyl]azetidine-1-carboxylate (2.0 g, 5 mmol, 1.0 eq) in THF (8.0 mL) and H₂O (8.0 mL) was added lithium hydroxide hydrate (569.0 mg, 13 mmol, 2.5 eq). The mixture was stirred at 25° C. for 10 h, then concentrated and diluted with water (20 mL). The pH of the mixture was adjusted with 2 N HCl until 5, and then lyophilized to afford 2-[3-[(1-tert-butoxycarbonylazetidin-3-yl)methoxy]isoxazol-5-yl]-3-methyl-butanoic acid (1.9 g) as a white solid, which was used in the next step without further purification. MS (ESI) m/z: 355.1 [M+1]⁺.

Step 3: Preparation of tert-butyl 3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]azetidine-1-carboxylate

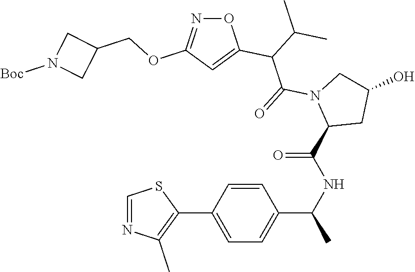 [SMILES: Cc1ncsc1-c1ccc([C@H](C)NC(=O)[C@@H]2C[C@@H](O)CN2C(=O)C(c2cc(OCC3CN(C(=O)OC(C)(C)C)C3)no2)C(C)C)cc1]

To a solution of 2-[3-[(1-tert-butoxycarbonylazetidin-3-yl)methoxy]isoxazol-5-yl]-3-methyl-butanoic acid (1.2 g, 3.4 mmol, 1.0 eq) in CH₂Cl₂ (20 mL) were added DIEA (3.0 mL, 6.9 mmol, 5.0 eq), HATU (1.67 g, 4.4 mmol, 1.3 eq) and (2S,4R)-4-hydroxy-N-[(1S)-1-[4-(4-methylthiazol-5-yl)phenyl]ethyl]pyrrolidine-2-carboxamide (1.99 g, 3.7 mmol, 1.1 eq). The mixture was stirred at 25° C. for 1 h, quenched by water (50 mL) and extracted with CH₂Cl₂ (70 mL×3). The combined organic layer was washed with brine (50 mL×2), dried over Na₂SO₄, filtered, and concentrated under reduced pressure. The residue was purified by flash column chromatography (0˜90% ethyl acetate in petroleum ether) afford tert-butyl 3-[[5-[1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]oxymethyl]azetidine-1-carboxylate (2.00 g, 88%) as a white solid. MS (ESI) m/z: 668.3 [M+1]⁺.

Step 4: Preparation of tert-butyl 3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]azetidine-1-carboxylate and tert-butyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]azetidine-1-carboxylate

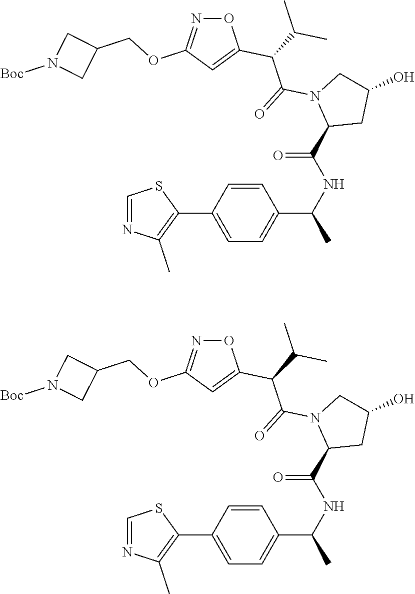 [SMILES: Cc1ncsc1-c1ccc([C@H](C)NC(=O)[C@@H]2C[C@@H](O)CN2C(=O)[C@H](c2cc(OCC3CN(C(=O)OC(C)(C)C)C3)no2)C(C)C)cc1 ; Cc1ncsc1-c1ccc([C@H](C)NC(=O)[C@@H]2C[C@@H](O)CN2C(=O)[C@@H](c2cc(OCC3CN(C(=O)OC(C)(C)C)C3)no2)C(C)C)cc1]

Racemic tert-butyl 3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy methyl]azetidine-1-carboxylate (2.0 g) was separated by chiral SFC {column: Daicel Chiralcel OX (250 mm*50 mm, 10 um); mobile phase: [0.1% NH₃H₂O, EtOH]; B %: 55%; gradient time: 60 min}.

Tert-butyl 3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]azetidine-1-carboxylate (1.0 g) was obtained as a yellow solid. MS (ESI) m/z: 668.2 [M+1]⁺.

Tert-butyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]azetidine-1-carboxylate (400 mg) was obtained as a yellow solid. MS (ESI) m/z: 668.2 [M+1]⁺.

Step 5: Preparation of (2S,4R)-1-[(2R)-2-[3-(azetidin-3-ylmethoxy)isoxazol-5-yl]-3-methyl-butanoyl]-4-hydroxy-N-[(1S)-1-[4-(4-methylthiazol-5-yl)phenyl]ethyl]pyrrolidine-2-carboxamide

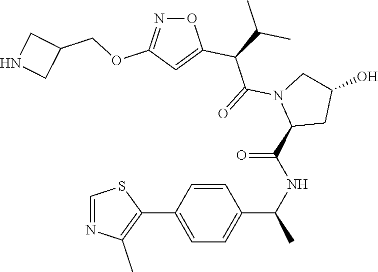 [SMILES: Cc1ncsc1-c1ccc([C@H](C)NC(=O)[C@@H]2C[C@@H](O)CN2C(=O)[C@@H](c2cc(OCC3CNC3)no2)C(C)C)cc1]

To a solution of tert-butyl 3-[[5-[(1R)-1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]oxymethyl]azetidine-1-carboxylate (200 mg, 0.3 mmol, 1.0 eq) in CH₂Cl₂ (6.0 mL) was added TFA (2.0 mL). The mixture was stirred at 25° C. for 30 min, then concentrated in vacuum to afford (2S,4R)-1-[(2R)-2-[3-(azetidin-3-ylmethoxy)isoxazol-5-yl]-3-methyl-butanoyl]-4-hydroxy-N-[(1S)-1-[4-(4-methylthiazol-5-yl)phenyl]ethyl]pyrrolidine-2-carboxamide trifluoroacetate (208 mg) as a yellow oil. MS (ESI) m/z: 568.2 [M+1]⁺.

Step 6: Preparation of tert-butyl 3-[7-[8-ethyl-3-(methoxymethoxy)-1-naphthyl]-8-fluoro-2-[[(3S,8S)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]azetidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

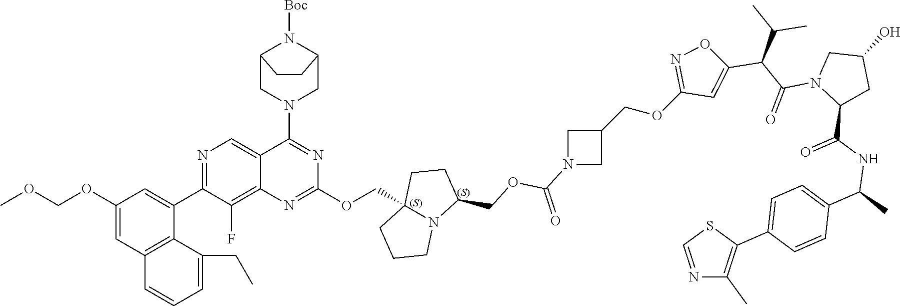

To a solution of (4-nitrophenyl) chloroformate (56.0 mg, 0.3 mmol, 1.9 eq) in THF (5.0 mL) was added triethylamine (0.165 mL, 1.2 mmol, 8.0 eq), DMAP (2.0 mg, 0.01 mmol, 0.10 eq) and tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (110 mg, 0.1 mmol, 1.0 eq). The reaction mixture was stirred at 30° C. for 15 h, to which (2S,4R)-1-[(2R)-2-[3-(azetidin-3-ylmethoxy)isoxazol-5-yl]-3-methyl-butanoyl]-4-hydroxy-N-[(1S)-1-[4-(4-methylthiazol-5-yl)phenyl]ethyl]pyrrolidine-2-carboxamide trifluoroacetate (151.0 mg, 0.2 mmol, 1.5 eq) was added. The reaction was stirred at 30° C. for 1 h, then concentrated. The residue was purified by flash column chromatography (0˜5% methanol in dichloromethane) to afford tert-butyl 3-[7-[8-ethyl-3-(methoxymethoxy)-1-naphthyl]-8-fluoro-2-[[(3S,8S)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]azetidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (180 mg, 80%) as yellow solid. MS (ESI) m/z: 1336.6 [M+1]⁺.

Step 7: Preparation of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-[({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)methyl]azetidine-1-carboxylate

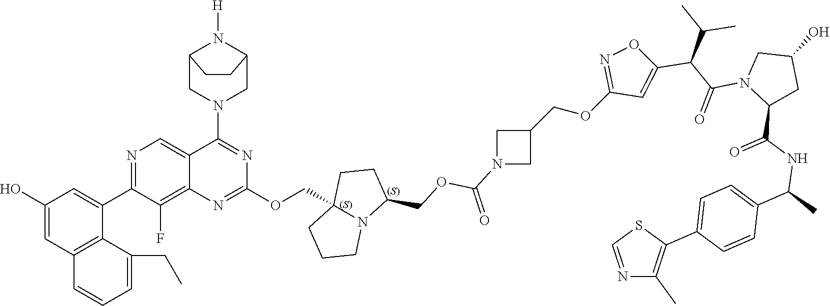

A solution of tert-butyl 3-[7-[8-ethyl-3-(methoxymethoxy)-1-naphthyl]-8-fluoro-2-[[(3S,8S)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy methyl]azetidine-1-carbonyl]oxymethyl]-1,2,3,5,6,7-hexahydropyrrolizin-8-yl]meth oxy]pyrido[4,3-d]pyrimidin-4-yl]-3,8-diazabicyclo[3.2.1]octane-8-carboxylate (180 mg, 0.1 mmol, 1.0 eq) in CH₂Cl₂ (3.0 mL) and TFA (1.0 mL) was stirred at 25° C. for 30 min, then concentrated, treated with saturated NaHCO₃ solution (20 mL), and extracted with CH₂Cl₂/MeOH (20 mL×3, v/v=10/1). The combined extracts were washed with brine (15 mL×3), dried over Na₂SO₄, filtered, and concentrated under reduced pressure. The residue was purified by preparative HPLC {column: Phenomenex C18 75*30 mm*3 um; mobile phase: [water (FA)-MeCN]; B %: 0-40; gradient time: 26 min} to afford [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-[({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)methyl]azetidine-1-carboxylate (formate, 50 mg, 47%) as white solid. MS (ESI) m/z: 1193.0 [M+1]⁺; 1H NMR (400 MHz, CD₃OD) δ 9.14-9.07 (m, 1H), 8.88 (s, 1H), 8.46 (s, 1H), 7.64 (d, J=8.4 Hz, 1H), 7.46-7.34 (m, 5H), 7.31-7.28 (m, 1H), 7.17 (d, J=5.2 Hz, 1H), 7.04-6.99 (m, 1H), 6.00 (s, 1H), 5.06-5.00 (m, 1H), 4.78-4.40 (m, 12H), 4.19-4.06 (m, 3H), 3.92-3.37 (m, 11H), 3.11-2.94 (m, 1H), 2.50-2.44 (m, 3H), 2.42-2.25 (m, 5H), 2.21-1.94 (m, 10H), 1.51 (d, J=7.2 Hz, 3H), 1.03 (d, J=6.4 Hz, 3H), 0.91-0.83 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-[({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)methyl]azetidine-1-carboxylate (Compound 115)

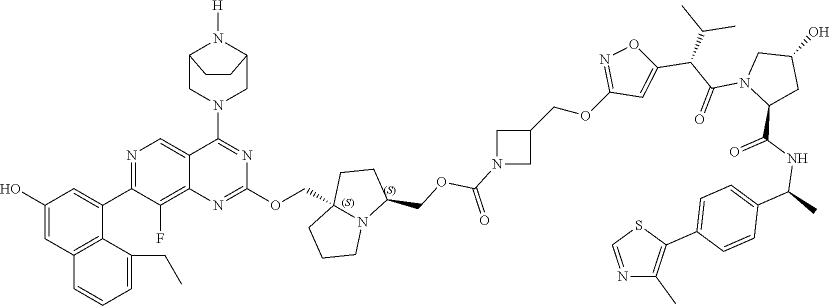

The title compound was prepared in an analogous manner to Compound 114 starting from (2S,4R)-1-[(2S)-2-[3-(azetidin-3-ylmethoxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). MS (ESI) m/z: 1193.0 [M+1]⁺; ¹H NMR (400 MHz, CD₃OD) δ 9.04 (s, 1H), 8.92-8.83 (m, 1H), 7.62 (d, J=7.6 Hz, 1H), 7.49-7.31 (m, 5H), 7.28 (d, J=2.4 Hz, 1H), 7.15 (d, J=6.8 Hz, 1H), 7.01 (d, J=2.4 Hz, 1H), 6.06-5.94 (m, 1H), 5.00-4.95 (m, 1H), 4.68-4.53 (m, 4H), 4.45-4.21 (m, 7H), 4.20-4.04 (m, 2H), 3.99-3.61 (m, 9H), 3.60-3.37 (m, 2H), 3.07-2.82 (m, 3H), 2.52-2.43 (m, 3H), 2.41-2.18 (m, 5H), 1.97-1.73 (m, 10H), 1.47 (d, J=7.2 Hz, 3H), 1.04 (d, J=6.4 Hz, 3H), 0.95-0.85 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)piperidine-1-carboxylate (Compound 116)

Step 1: Preparation of tert-butyl 4-[[5-(1-methoxycarbonyl-2-methyl-propyl)isoxazol-3-yl]oxypiperidine-1-carboxylate

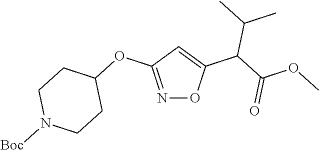 [SMILES: COC(=O)C(c1cc(OC2CCN(C(=O)OC(C)(C)C)CC2)no1)C(C)C]

To a mixture of methyl 2-(3-hydroxyisoxazol-5-yl)-3-methyl-butanoate (1.0 g, 5.02 mmol, 1.0 eq), tert-butyl 4-hydroxypiperidine-1-carboxylate (1.01 g, 5.0 mmol, 1.0 eq) and PPh₃ (2.11 g, 8.0 mmol, 1.6 eq) in anhydrous toluene (20 mL) was added DIAD (1.6 mL, 8.0 mmol, 1.6 eq) dropwise at 20° C. under N₂. The resulting suspension was stirred at 110° C. for 12 h, then concentrated. The residue was purified by column chromatography (0˜5% to 10% ethyl acetate in petroleum ether) to afford tert-butyl 4-[[5-(1-methoxycarbonyl-2-methyl-propyl)isoxazol-3-yl]oxypiperidine-1-carboxylate (1.6 g, 73%) as colorless gum. MS (ESI) m/z: 327.3 [M−55]⁺; ¹H NMR (400 MHz, CDCl₃) δ 5.88 (s, 1H), 4.79 (tt, J=8.0, 3.6 Hz, 1H), 3.77-3.71 (m, 5H), 3.49 (d, J=8.8 Hz, 1H), 3.30-3.22 (m, 2H), 2.35 (q, J=8.4, 6.8 Hz, 1H), 2.02 (td, J=3.6, 9.6 Hz, 2H), 1.76 (tdd, J=4.2, 8.4, 12.6 Hz, 2H), 1.47 (s, 9H), 1.00 (d, J=6.8 Hz, 3H), 0.93 (d, J=6.8 Hz, 3H).

Step 2: Preparation of 2-[3-[(1-tert-butoxycarbonyl-4-piperidyl)oxy]isoxazol-5-yl]-3-methyl-butanoic acid

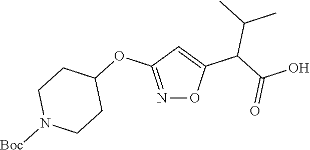 [SMILES: CC(C)C(C(=O)O)c1cc(OC2CCN(C(=O)OC(C)(C)C)CC2)no1]

To a mixture of tert-butyl 4-[[5-(1-methoxycarbonyl-2-methyl-propyl)isoxazol-3-yl]oxypiperidine-1-carboxylate (2.4 g, 6.3 mmol, 1.0 eq) in THF (30 mL) and H₂O (10 mL) was added lithium hydroxide hydrate (1.32 g, 31.4 mmol, 5.0 eq) at 20° C. The mixture was stirred at 20° C. for 12 h, then concentrated under reduced pressure. The residue was diluted with water (20 mL) and adjusted the pH with 2 M hydrochloric acid to 3. The resulting mixture was extracted with dichloromethane (40 mL×3). The combined organic phase was washed with brine (30 mL), dried with anhydrous Na₂SO₄, filtered, and concentrated to afford 2-[3-[(1-tert-butoxycarbonyl-4-piperidyl)oxy]isoxazol-5-yl]-3-methyl-butanoic acid (2.3 g, 76%) as a yellow gum. MS (ESI) m/z: 313.0 [M−55]⁺.

Step 3: Preparation of tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate

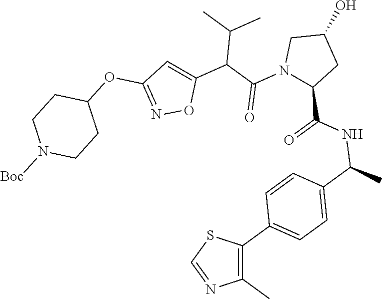 [SMILES: Cc1ncsc1-c1ccc([C@H](C)NC(=O)[C@@H]2C[C@@H](O)CN2C(=O)C(c2cc(OC3CCN(C(=O)OC(C)(C)C)CC3)no2)C(C)C)cc1]

To a mixture of 2-[3-[(1-tert-butoxycarbonyl-4-piperidyl)oxy]isoxazol-5-yl]-3-methyl-butanoic acid (1.5 g, 4.1 mmol, 1.0 eq) and (2S,4R)-4-hydroxy-N-[(1S)-1-[4-(4-methylthiazol-5-yl)phenyl]ethyl]pyrrolidine-2-carboxamide hydrochloride (2.66 g, 4.5 mmol, 1.1 eq) in anhydrous dichloromethane (30 mL) were added DIEA (3.6 mL, 20.4 mmol, 5.0 eq) and HATU (1.7 g, 4.5 mmol, 1.1 eq) at 20° C. The mixture was stirred at 20° C. for 12 h, then quenched by water (30 mL) and extracted with dichloromethane (40 mL×3). The combined organic phase was washed with brine (30 mL), dried over anhydrous Na₂SO₄, filtered and concentrated. The residue was purified by flash column chromatography (0˜100% ethyl acetate in petroleum ether) to afford tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate (2.6 g, 90%) as a yellow solid. MS (ESI) m/z: 682.4 [M+H]⁺. A mixture of tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate (1.4 g, 2.1 mmol) and Cs₂CO₃ (669 mg, 2.1 mmol, 1.0 eq) in dioxane (34 mL) was stirred at 70° C. for 12 h, the mixture was filtered, the filtrate was concentrated to give tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate (1.4 g, 87%) as a yellow solid. MS (ESI) m/z: 682.5 [M+H]⁺.

Step 4: Preparation of tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate and tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate

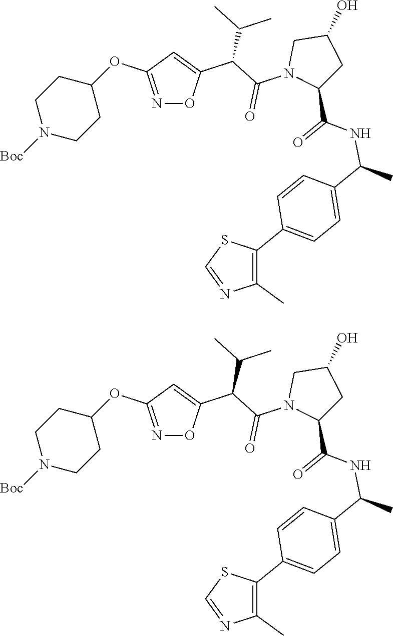

Tert-butyl 4-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate (1.6 g) was separated by SFC (column: Daicel Chiralpak IA (250 mm*30 mm, 10 um); mobile phase: [0.1% NH₃H₂O isopropanol]; B %: 30%).

Tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate (769 mg) was obtained as a yellow solid. MS (ESI) m/z: 682.1 [M+H]⁺.

Tert-butyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate (777 mg) was obtained as a white solid. MS (ESI) m/z: 682.1 [M+H]⁺.

Step 5: Preparation of (2S,4R)-4-hydroxy-1-[(2R)-3-methyl-2-[3-(4-piperidyloxy)isoxazol-5-yl]butanoyl]-N-[(1S)-1-[4-(4-methylthiazol-5-yl)phenyl]ethyl]pyrrolidine-2-carboxamide

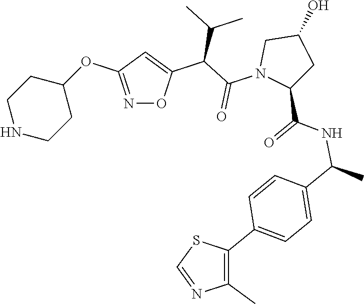 [SMILES: Cc1ncsc1-c1ccc([C@H](C)NC(=O)[C@@H]2C[C@@H](O)CN2C(=O)[C@@H](c2cc(OC3CCNCC3)no2)C(C)C)cc1]

To a mixture of tert-butyl 4-[[5-[(1R)-1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]oxypiperidine-1-carboxylate (250 mg, 0.4 mmol, 1.0 eq) in anhydrous dichloromethane (2.0 mL) was added 4 M HCl/dioxane (1.4 mL, 15.0 eq) at 20° C. The mixture was stirred at 20° C. for 30 min, then concentrated to give (2S,4R)-4-hydroxy-1-[(2R)-3-methyl-2-[3-(4-piperidyl oxy)isoxazol-5-yl]butanoyl]-N-[(1S)-1-[4-(4-methylthiazol-5-yl)phenyl]ethyl]pyrrolidine-2-carboxamide (210 mg, crude) as yellow gum. MS (ESI) m/z: 582.4 [M+H]⁺.

Step 6: Preparation of tert-butyl 3-[7-[8-ethyl-3-(methoxy methoxy)-1-naphthyl]-8-fluoro-2-[[(3S,8R)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

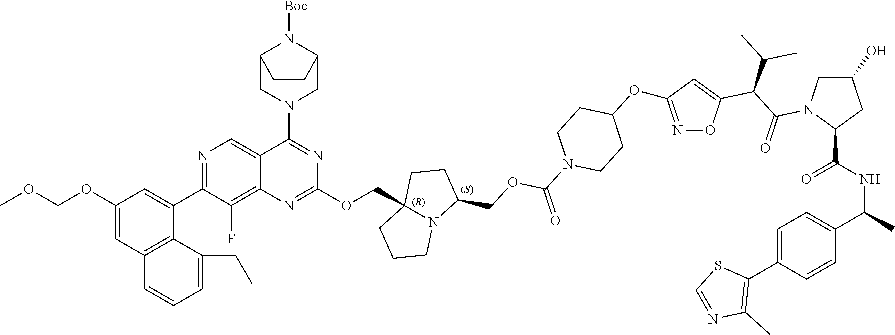

To a reaction mixture of tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (180 mg, 0.2 mmol, 1.0 eq) in anhydrous THF (6.0 mL) were added triethylamine (0.27 mL, 1.9 mmol, 8.0 eq), DMAP (8.88 mg, 0.07 mmol, 0.3 eq) and (4-nitrophenyl) chloroformate (73 mg, 0.4 mmol, 1.5 eq). The resulting suspension was stirred at 28° C. for 15 h, then DMAP (9.0 (2S,4R)-4-hydroxy-1-[(2R)-3-methyl-2-[3-(4-mg, 0.07 mmol, 0.30 eq) and piperidyloxy)isoxazol-5-yl]butanoyl]-N-[(1S)-1-[4-(4-methylthiazol-5-yl)phenyl]ethyl]pyrrolidine-2-carboxamide hydrochloride (210 mg, 0.3 mmol, 1.4 eq) were added. The resulting suspension was stirred at 28° C. for 1 h, filtered, and washed with tetrahydrofuran (10 mL×3). The filtrate solution was concentrated. The residue was purified by flash column chromatography (0˜4% to 8% methanol (1N NH₃ as additive) in dichloromethane) to afford tert-butyl 3-[7-[8-ethyl-3-(methoxy methoxy)-1-naphthyl]-8-fluoro-2-[[(3S,8R)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (272 mg, 68%) as a yellow solid. MS (ESI) m/z: 1350.8 [M+H]⁺.

Step 7: Preparation of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)piperidine-1-carboxylate

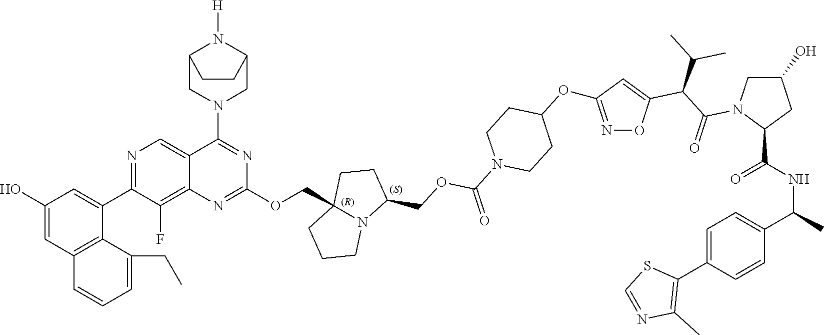

To a mixture of tert-butyl 3-[7-[8-ethyl-3-(methoxymethoxy)-1-naphthyl]-8-fluoro-2-[[(3S,8R)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (272 mg, 0.2 mmol, 1.0 eq) in dichloromethane (2.0 mL) was added 4 M HCl/dioxane (1.01 mL) at 20° C. The mixture was stirred at 20° C. for 30 min, then concentrated. The pH of the residue was adjusted to 9 by triethylamine and suspended in tetrahydrofuran, filtered and concentrated. The crude residue was purified by preparative HPLC (column: Phenomenex C18 75*30 mm*3 um; mobile phase: [water (FA)-MeCN]; B %: 6-46; 28 min) to afford [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)piperidine-1-carboxylate (formate, 156.5 mg, 63%) as a white solid. MS (ESI) m/z: 1207.0 [M+H]⁺; ¹H NMR (400 MHz, CD₃OD) δ 9.11-9.06 (m, 1H), 8.91-8.84 (m, 1H), 8.50 (s, 1H), 7.64 (d, J=8.0 Hz, 1H), 7.45-7.34 (m, 5H), 7.32-7.27 (m, 1H), 7.16 (d, J=6.4 Hz, 1H), 7.03 (dd, J=4.4, 2.8, Hz, 1H), 6.03-5.95 (m, 1H), 5.05-4.99 (m, 1H), 4.72 (dd, J=7.6, 4.0 Hz, 3H), 4.61 (s, 3H), 4.51 (td, J=4.0, 8.4 Hz, 1H), 4.43-4.38 (m, 2H), 4.27-4.17 (m, 1H), 4.15-4.06 (m, 1H), 3.90-3.79 (m, 4H), 3.81-3.61 (m, 6H), 3.10-2.97 (m, 1H), 2.49-2.46 (m, 3H), 2.40-2.27 (m, 4H), 2.21-2.14 (m, 1H), 2.11-2.06 (m, 1H), 2.04-1.84 (m, 14H), 1.75-1.66 (m, 2H), 1.54-1.47 (m, 3H), 1.05 (d, J=6.4 Hz, 3H), 0.92-0.87 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)piperidine-1-carboxylate (Compound 117)

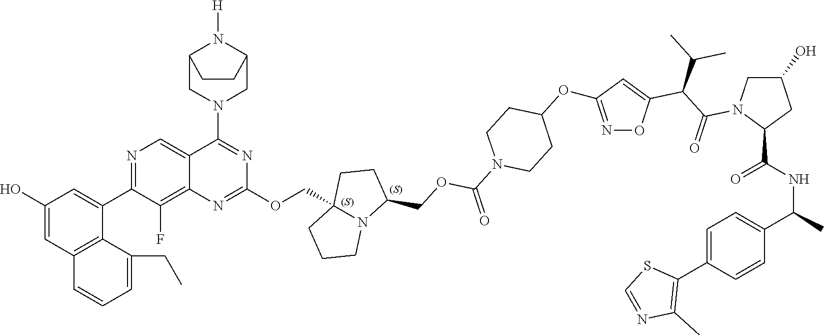

The title compound was prepared in an analogous manner to Compound 116 starting from tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate. (white solid) MS (ESI) m/z: 1206.9 [M+1]⁺; ¹H NMR (400 MHz, CD₃OD) δ 9.16-9.04 (m, 1H), 8.86 (s, 1H), 8.42 (s, 2H), 8.38-8.17 (m, 1H), 7.62 (d, J=7.6 Hz, 1H), 7.54-7.31 (m, 5H), 7.29 (d, J=2.4 Hz, 1H), 7.19-7.09 (m, 1H), 7.02-6.96 (m, 1H), 5.98 (s, 1H), 5.01 (q, J=6.8 Hz, 1H), 4.76-4.63 (m, 6H), 4.62-4.51 (m, 4H), 4.45-4.31 (m, 2H), 4.18 (dt, J=3.6, 6.0 Hz, 1H), 3.96-3.88 (m, 2H), 3.85-3.71 (m, 4H), 3.69-3.57 (m, 2H), 3.54-3.42 (m, 2H), 2.51-2.43 (m, 3H), 2.41-2.25 (m, 5H), 2.23-2.14 (m, 3H), 2.12-1.87 (m, 11H), 1.82-1.63 (m, 2H), 1.59-1.43 (m, 3H), 1.03 (d, J=6.4 Hz, 3H), 0.92 (d, J=3.2 Hz, 1H), 0.91-0.84 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)piperidine-1-carboxylate (Compound 118)

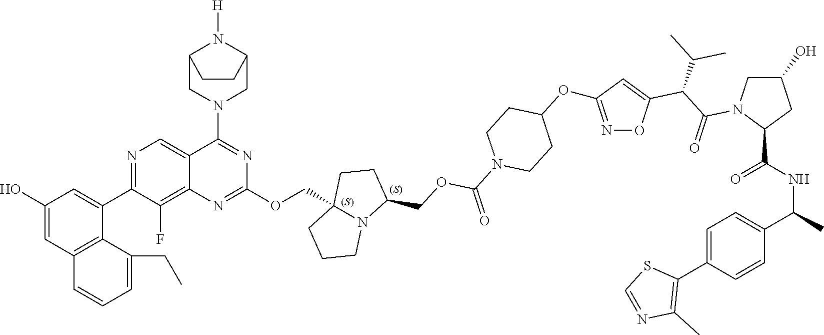

The title compound was prepared in an analogous manner to Compound 116 starting from tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate. (formate, white solid). MS (ESI) m/z: 1207.0 [M+H]⁺; ¹H NMR (400 MHz, CD₃OD) δ 9.13-9.03 (m, 1H), 8.92-8.84 (m, 1H), 8.48 (s, 1H), 7.63 (d, J=8.4 Hz, 1H), 7.47-7.27 (m, 6H), 7.16 (dd, J=6.4, 3.6 Hz, 1H), 7.03-6.95 (m, 1H), 6.06-5.96 (m, 1H), 5.01 (d, J=6.8 Hz, 1H), 4.77-4.63 (m, 6H), 4.60-4.51 (m, 3H), 4.47-4.36 (m, 2H), 4.27-4.14 (m, 1H), 3.88 (s, 3H), 3.81-3.63 (m, 6H), 3.54 (s, 1H), 2.50-2.40 (m, 4H), 2.38-2.15 (m, 8H), 2.14-1.85 (m, 11H), 1.73-1.56 (m, 2H), 1.53-1.41 (m, 3H), 1.05 (d, J=6.4 Hz, 3H), 0.93-0.83 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 4-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)piperidine-1-carboxylate (Compound 119)

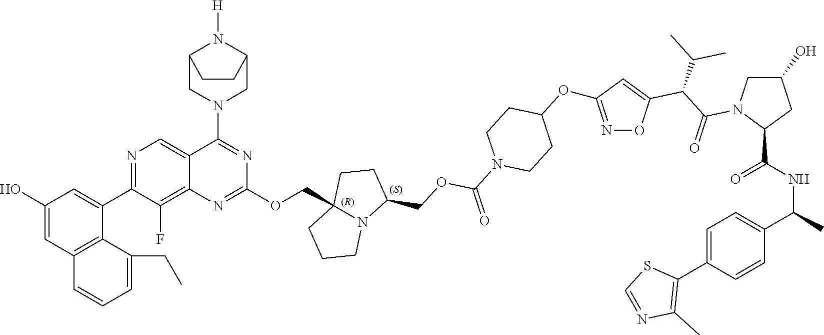

The title compound was prepared in an analogous manner to Compound 116 starting from (2S,4R)-4-hydroxy-1-[(2S)-3-methyl-2-[3-(4-piperidyloxy)isoxazol-5-yl]butanoyl]-N-[(1S)-1-[4-(4-methylthiazol-5-yl)phenyl]ethyl]pyrrolidine-2-carboxamide. (formate, white solid). MS (ESI) m/z: 1207.0. [M+1]⁺; ¹H NMR (400 MHz, CD₃OD) δ 9.10-9.09 (m, 1H), 8.88 (s, 1H), 7.62 (d, J=8.0 Hz, 1H), 7.48-7.29 (m, 6H), 7.15 (d, J=8.0 Hz, 1H), 7.02-7.01 (m, 1H), 6.05-5.97 (m, 1H), 4.98 (d, J=4.4 Hz, 1H), 4.61-4.54 (m, 2H), 4.51-4.46 (m, 2H), 4.41-4.28 (m, 2H), 4.19-4.14 (m, 1H), 4.06-4.03 (m, 2H), 3.92-3.81 (m, 2H), 3.77 (d, J=8.8 Hz, 1H), 3.73-3.60 (m, 4H), 3.53-3.47 (m, 1H), 3.38-3.34 (m, 1H), 3.27-3.20 (m, 2H), 2.49-2.46 (m, 3H), 2.40-2.13 (m, 7H), 2.08-1.86 (m, 13H), 1.66-1.57 (m, 2H), 1.47 (d, J=6.8 Hz, 1H), 1.05 (d, J=6.4 Hz, 1H), 0.95-0.84 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (2R)-4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2-methylpiperazine-1-carboxylate (Compound 120)

Step 1: Preparation of tert-butyl(2R)-4-[5-(1-methoxycarbonyl-2-methyl-propyl)isoxazol-3-yl]-2-methyl-piperazine-1-carboxylate

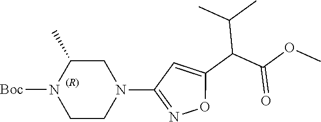

To a mixture of tert-butyl(2R)-2-methylpiperazine-1-carboxylate (1.6 g, 8.3 mmol, 1.0 eq) and 4 A MS (4.0 g, 8.3 mmol, 1.0 eq) in N,N-dimethylacetamide (40 mL) were added triethylamine (3.5 mL, 24.9 mmol, 3.0 eq) and methyl 3-methyl-2-[3-(1,1,2,2,3,3,4,4,4-nonafluorobutylsulfonyloxy)isoxazol-5-yl]butanoate (4.0 g, 8.3 mmol, 1.0 eq). The mixture was stirred at 130° C. for 16 h, diluted with water (50 mL) and extracted with ethyl acetate (40 mL×5). The combined organic phase was dried over anhydrous Na₂SO₄, filtered, and concentrated under reduced pressure. The residue was purified by flash column chromatography (0˜17% ethyl acetate in petroleum ether) to afford tert-butyl(2R)-4-[5-(1-methoxycarbonyl-2-methyl-propyl)isoxazol-3-yl]-2-methyl-piperazine-1-carboxylate (2.09 g, 61%) as light yellow oil. MS (ESI) m/z: 382.2 [M+H]⁺.

Step 2: Preparation of 2-[3-[(3R)-4-tert-butoxycarbonyl-3-methyl-piperazin-1-yl]isoxazol-5-yl]-3-methyl-butanoic acid

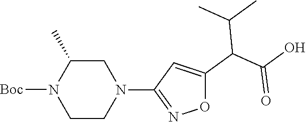 [SMILES: CC(C)C(C(=O)O)c1cc(N2CCN(C(=O)OC(C)(C)C)[C@H](C)C2)no1]

To a stirred solution of tert-butyl(2R)-4-[5-(1-methoxycarbonyl-2-methyl-propyl) isoxazol-3-yl]-2-methyl-piperazine-1-carboxylate (2.09 g, 5.5 mmol, 1.0 eq) in tetrahydrofuran (20 mL) and water (10 mL) was added lithium hydroxide hydrate (805 mg, 19.2 mmol, 3.5 eq). The mixture was stirred at 30° C. for 16 h, then concentrated. The residue was acidified with 2 M hydrochloric acid till pH 3-4. The mixture was extracted with dichloromethane (20 mL×3). The combined organic layers were washed with brine (15 mL), dried over anhydrous Na₂SO₄, filtered and concentrated under reduced pressure to afford 2-[3-[(3R)-4-tert-butoxycarbonyl-3-methyl-piperazin-1-yl]isoxazol-5-yl]-3-methyl-butanoic acid (1.9 g, crude) as light yellow oil. MS (ESI) m/z: 368.3 [M+H]⁺.

Step 3: Preparation of tert-butyl(2R)-4-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate

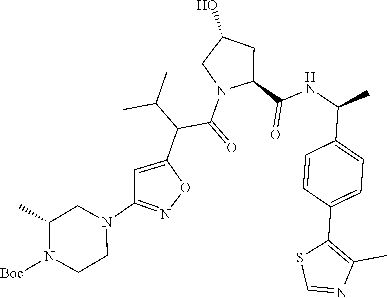

To a solution of 2-[3-[(3R)-4-tert-butoxycarbonyl-3-methyl-piperazin-1-yl]isoxazol-5-yl]-3-methyl-butanoic acid (1.90 g, 5.2 mmol, 1.0 eq) and (2S,4R)-4-hydroxy-N-[(1S)-1-[4-(4-methylthiazol-5-yl)phenyl]ethyl]pyrrolidine-2-carboxamide (3.0 g, 5.7 mmol, 1.1 eq) in dichloromethane (20 mL) were added DIEA (4.5 mL, 25.9 mmol, 5.0 eq) and HATU (2.2 g, 5.7 mmol, 1.1 eq). The mixture was stirred at 25° C. for 2 h, diluted with dichloromethane (20 mL), washed with water (30 mL), saturated NaHCO₃ solution (30 mL) and brine (30 mL). The organic phase was dried over anhydrous Na₂SO₄, filtered, and concentrated under reduced pressure. The residue was purified by flash column chromatography (0˜100% ethyl acetate in petroleum ether) to afford tert-butyl(2R)-4-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate (3.20 g, 80%) as a white solid. MS (ESI) m/z: 681.4 [M+H]⁺; ¹H NMR (400 MHz, DMSO-d₆) δ 8.99-8.97 (m, 1H), 7.43-7.33 (m, 3H), 6.19-6.17 (m, 1H), 4.93-4.84 (m, 1H), 4.28-4.27 (m, 1H), 4.17-4.16 (m, 1H), 3.77-3.69 (m, 2H), 3.64-3.52 (m, 4H), 3.15-3.04 (m, 3H), 2.93-2.86 (m, 1H), 2.72-2.65 (m, 1H), 2.46-2.44 (m, 3H), 1.41-1.40 (m, 9H), 1.37-1.34 (m, 1H), 1.27-1.22 (m, 6H), 1.14-1.12 (m, 2H), 1.09-1.07 (m, 2H), 0.96-0.94 (m, 3H), 0.83-0.78 (m, 3H).

Step 4: Preparation of tert-butyl(2R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate and tert-butyl(2R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate

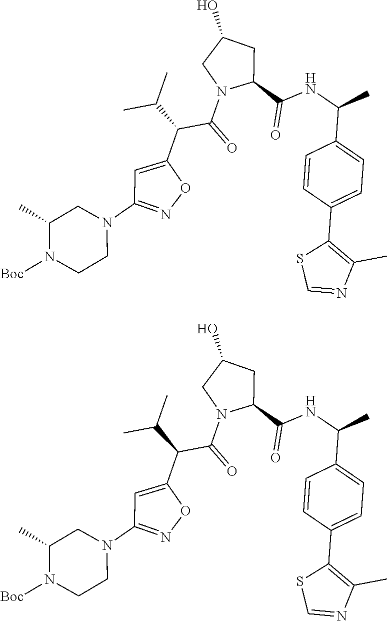

Racemic tert-butyl(2R)-4-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate (900 mg) was separated by chiral SFC (column: Daicel ChiralPak IG (250*30 mm, 10 um); mobile phase: [0.1% NH₃H₂O-EtOH]; B %: 40%).

Tert-butyl(2R)-4-[5-[(1S)-1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]-2-methyl-piperazine-1-carboxylate (506 mg) was obtained as a white solid. MS (ESI) m/z: 681.4 [M+H]⁺.

Tert-butyl(2R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate (227 mg) was obtained as a white solid. MS (ESI) m/z: 681.4 [M+H]⁺.

Step 5: Preparation of (2S,4R)-4-hydroxy-1-[(2R)-3-methyl-2-[3-[(3R)-3-methylpiperazin-1-yl]isoxazol-5-yl]butanoyl]-N-[(1S)-1-[4-(4-methylthiazol-5-yl)phenyl]ethyl]pyrrolidine-2-carboxamide

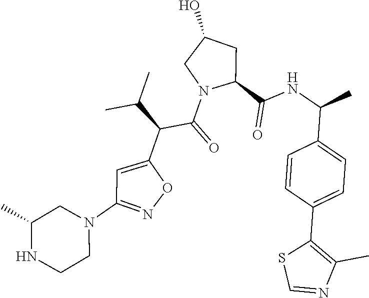

To a stirred solution of tert-butyl(2R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate (227 mg, 0.3 mmol, 1.0 eq) in dichloromethane (2.5 mL) was added 4 M HCl/dioxane (2.5 mL). The reaction was stirred at 20° C. for 1 h, then concentrated to afford (2S,4R)-4-hydroxy-1-[(2R)-3-methyl-2-[3-[(3R)-3-methylpiperazin-1-yl]isoxazol-5-yl]butanoyl]-N-[(1S)-1-[4-(4-methylthiazol-5-yl)phenyl]ethyl]pyrrolidine-2-carboxamide hydrochloride (376 mg) as a light yellow solid. MS (ESI) m/z: 581.2 [M+H]⁺.

Step 6: Preparation of tert-butyl 3-[7-[8-ethyl-3-(methoxy methoxy)-1-naphthyl]-8-fluoro-2-[[(3S,8R)-3-[[(2R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

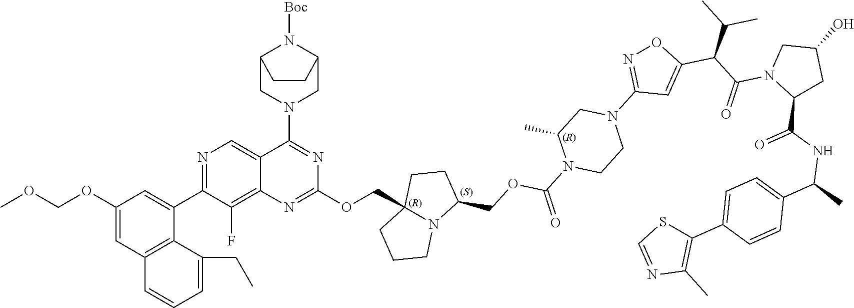

To a solution of tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (200 mg, 0.3 mmol, 1.0 eq) and (4-nitrophenyl) chloroformate (79 mg, 0.4 mmol, 1.45 eq) in tetrahydrofuran (4.0 mL) were added triethylamine (0.30 mL, 2.2 mmol, 8.0 eq) and DMAP (10 mg, 0.08 mmol, 0.30 eq). The mixture was stirred at 25° C. for 15 h, then (2S,4R)-4-hydroxy-1-[(2R)-3-methyl-2-[3-[(3R)-3-methylpiperazin-1-yl]isoxazol-5-yl]butanoyl]-N-[(1S)-1-[4-(4-methylthiazol-5-yl)phenyl]ethyl]pyrrolidine-2-carboxamide hydrochloride (233 mg, 0.4 mmol, 1.4 eq) and triethylamine (0.15 mL, 1 mmol, 3.0 eq) were added. The resulting mixture was stirred at 25° C. for 1 hour. The reaction mixture was filtered and the filtrate was concentrated under reduced pressure. The residue was purified by flash column chromatography (0˜5% methanol in dichloromethane) to afford tert-butyl 3-[7-[8-ethyl-3-(methoxymethoxy)-1-naphthyl]-8-fluoro-2-[[(3S,8R)-3-[[(2R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (150 mg, 35%) as a light yellow solid. MS (ESI) m/z: 1349.7 [M+H]⁺.

Step 7: Preparation of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (2R)-4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2-methylpiperazine-1-carboxylate

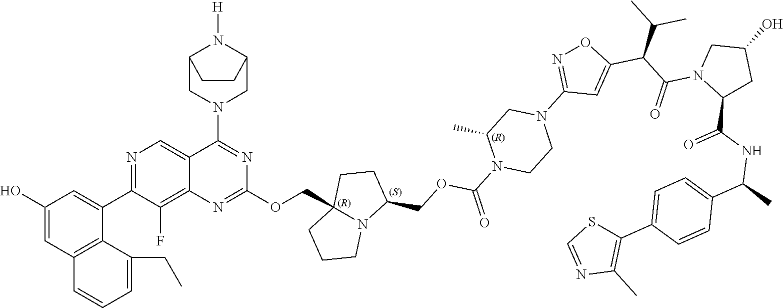

To a stirred solution of tert-butyl 3-[7-[8-ethyl-3-(methoxymethoxy)-1-naphthyl]-8-fluoro-2-[[(3S,8R)-3-[[(2R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (150 mg, 0.1 mmol, 1.0 eq) in dichloromethane (1.0 mL) was added 4 M HCl/dioxane (1.1 mL). The reaction was stirred at 20° C. for 1 h. The reaction mixture was diluted with petroleum ether (5.0 mL) and filtered. The filtrate was concentrated, the residue was re-dissolved in tetrahydrofuran (20 mL) and basified with triethylamine until pH 8. The mixture was filtered, and the filtrate was concentrated. The residual gum was purified by preparative HPLC {column: Phenomenex C18 75*30 mm*3 um; mobile phase: [water (FA)-MeCN]; B %: 8-48; 26 min} to afford [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (2R)-4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2-methylpiperazine-1-carboxylate (formate, 81.8 mg, 58%) as a white solid. MS (ESI) m/z: 1205.4 [M+H]⁺; ¹H NMR (400 MHz, CD₃OD) δ 9.13-9.11 (m, 1H), 8.88 (s, 1H), 7.64-7.63 (m, 1H), 7.43-7.37 (m, 5H), 7.31-7.29 (m, 1H), 7.18-7.15 (m, 1H), 7.03-7.02 (m, 1H), 6.08-6.07 (m, 1H), 5.04-5.02 (m, 1H), 4.53-4.43 (m, 4H), 4.33-4.28 (m, 2H), 4.18-4.14 (m, 1H), 4.04-4.02 (m, 2H), 3.92-3.81 (m, 4H), 3.65-3.37 (m, 6H), 3.23-3.13 (m, 2H), 2.99-2.91 (m, 1H), 2.84-2.72 (m, 1H), 2.47 (s, 3H), 2.40-2.25 (m, 5H), 2.17-1.91 (m, 14H), 1.52-1.49 (m, 3H), 1.21-1.18 (m, 3H), 1.06-1.04 (m, 3H), 0.91-0.86 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-[({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)methyl]azetidine-1-carboxylate (Compound 121)

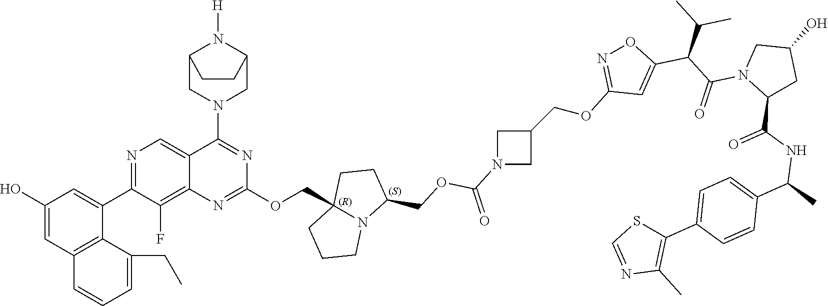

The title compound was prepared in an analogous manner to Compound 114 starting from tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate. (formate, white solid). MS (ESI) m/z: 1193.0 [M+1]⁺; 1H NMR (400 MHz, MeOD) δ 9.09 (d, J=2.8 Hz, 1H), 8.93-8.82 (m, 1H), 8.45 (s, 1H), 7.63 (d, J=8.0 Hz, 1H), 7.45-7.32 (m, 5H), 7.31-7.27 (m, 1H), 7.17 (d, J=6.4 Hz, 1H), 7.05-7.00 (m, 1H), 6.05-5.87 (m, 1H), 5.02 (q, J=7.2 Hz, 1H), 4.79-4.66 (m, 3H), 4.62 (s, 1H), 4.54-4.39 (m, 4H), 4.31-4.20 (m, 3H), 4.16-4.01 (m, 3H), 3.99-3.68 (m, 8H), 3.66-3.49 (m, 2H), 3.18-2.93 (m, 3H), 2.51-2.44 (m, 3H), 2.41-2.18 (m, 5H), 2.05-1.87 (m, 10H), 1.60-1.47 (m, 3H), 1.09-1.01 (m, 3H), 0.93-0.85 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-[({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)methyl]azetidine-1-carboxylate (Compound 122)

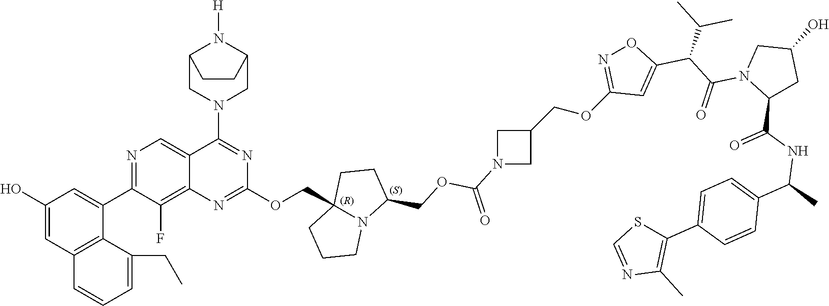

The title compound was prepared in an analogous manner to Compound 114 starting from tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate. (white solid). MS (ESI) m/z: 1193.0 [M+1]⁺; ¹H NMR (400 MHz, CD₃OD) δ 9.03 (s, 1H), 8.91-8.84 (m, 1H), 7.65-7.58 (m, 1H), 7.47-7.33 (m, 5H), 7.30-7.25 (m, 1H), 7.15 (d, J=6.9 Hz, 1H), 7.02 (dd, J=2.8, 6.4 Hz, 1H), 6.05-5.93 (m, 1H), 5.03-4.99 (m, 1H), 4.67-4.53 (m, 6H), 4.32-4.06 (m, 7H), 3.95-3.80 (m, 3H), 3.80-3.60 (m, 8H), 3.09-2.97 (m, 3H), 2.51-2.45 (m, 3H), 2.45-2.19 (m, 5H), 1.95-1.75 (m, 10H), 1.48 (dd, J=2.0, 6.8 Hz, 3H), 1.05 (d, J=6.4 Hz, 3H), 0.93-0.87 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (3S)-4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-3-methylpiperazine-1-carboxylate (Compound 123)

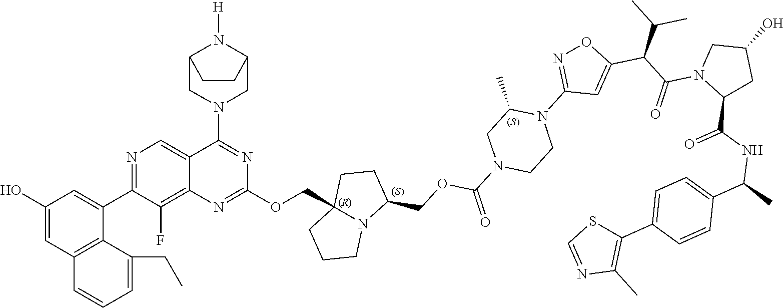

The title compound was prepared in an analogous manner to Compound 120 starting from tert-butyl(3S)-3-methyl piperazine-1-carboxylate. (formate, white solid). MS (ESI) m/z: 1205.4 [M+1]⁺; ¹H NMR (400 MHz, CD₃OD) δ 9.09 (s, 1H), 8.89 (s, 1H), 8.53 (s, 1H), 7.65 (d, J=7.2 Hz, 1H), 7.49-7.34 (m, 5H), 7.31 (d, J=2.8 Hz, 1H), 7.18-7.16 (m, 1H), 7.05 (s, 1H), 6.13-6.04 (m, 1H), 5.07-5.02 (m, 2H), 4.75-4.63 (m, 3H), 4.60-4.51 (m, 2H), 4.43 (s, 1H), 4.37 (s, 1H), 4.20-4.06 (m, 3H), 3.90-3.72 (m, 7H), 3.64-3.60 (m, 1H), 3.52-3.47 (m, 1H), 3.22-3.05 (m, 4H), 2.49 (s, 3H), 2.38-1.84 (m, 18H), 1.59-1.49 (m, 3H), 1.14-1.05 (m, 6H), 0.94-0.85 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (3S)-4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-3-methylpiperazine-1-carboxylate (Compound 124)

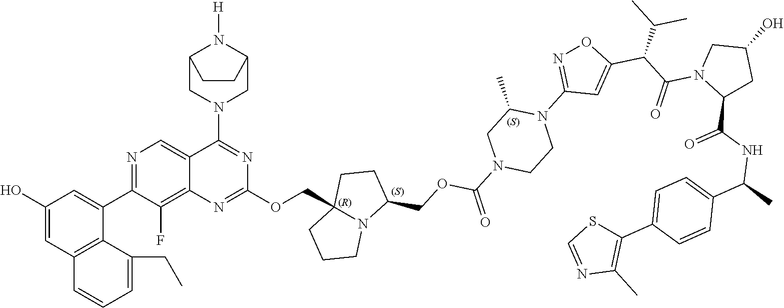

The title compound was prepared in an analogous manner to Compound 120 starting from tert-butyl(3S)-3-methyl piperazine-1-carboxylate. (white solid). MS (ESI) m/z: 1206.0 [M+1]⁺; 1H NMR (400 MHz, CD₃OD) δ 9.05 (d, J=3.2 Hz, 1H), 8.86 (s, 1H), 8.51 (s, 1H), 7.63-7.59 (m, 1H), 7.47-7.32 (m, 5H), 7.30-7.25 (m, 1H), 7.15 (d, J=7.2 Hz, 1H), 7.03-7.01 (m, 1H), 6.15-6.02 (m, 1H), 4.98-4.96 (m, 2H), 4.72-4.65 (m, 2H), 4.63-4.52 (m, 3H), 4.42 (s, 1H), 4.34 (s, 1H), 4.20-4.04 (m, 3H), 3.87-3.71 (m, 7H), 3.68 (s, 1H), 3.65-3.60 (m, 1H), 3.19-3.05 (m, 4H), 2.47 (s, 3H), 2.42-2.18 (m, 6H), 2.09-1.82 (m, 12H), 1.59-1.46 (m, 3H), 1.12-1.01 (m, 6H), 0.95-0.86 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (2R)-4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2-methylpiperazine-1-carboxylate (Compound 125)

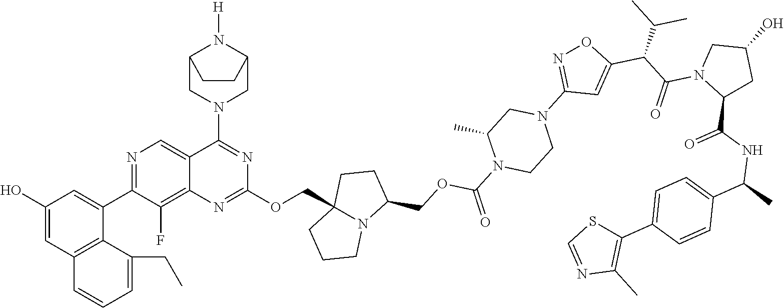 [SMILES: CCc1cccc2cc(O)cc(-c3ncc4c(N5CC6CCC(C5)N6)nc(OC[C@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)C[C@H]5C)CC6)nc4c3F)c12]

The title compound was prepared in an analogous manner to Compound 120 starting from (2S,4R)-4-hydroxy-1-[(2S)-3-methyl-2-[3-[(3R)-3-methylpiperazin-1-yl]isoxazol-5-yl]butanoyl]-N-[(1S)-1-[4-(4-methylthiazol-5-yl)phenyl]ethyl]pyrrolidine-2-carboxamide. (formate, white solid). MS (ESI) m/z: 1205.4 [M+H]⁺; ¹H NMR (400 MHz, CD₃OD) δ 9.10-9.09 (m, 1H), 8.86 (s, 1H), 7.63-7.61 (m, 1H), 7.42-7.39 (m, 2H), 7.37-7.34 (m, 3H), 7.29-7.26 (m, 1H), 7.16-7.14 (m, 1H), 7.03-7.01 (m, 1H), 6.14-6.12 (m, 1H), 4.99-4.95 (m, 1H), 4.61-4.56 (m, 1H), 4.50-4.42 (m, 3H), 4.31-4.26 (m, 2H), 4.17-4.13 (m, 1H), 4.05-4.02 (m, 2H), 3.90-3.82 (m, 3H), 3.77-3.66 (m, 3H), 3.60-3.54 (m, 1H), 3.49-3.40 (m, 2H), 3.37-3.34 (m, 1H), 3.20-3.13 (m, 2H), 2.96-2.92 (m, 1H), 2.81-2.74 (m, 1H), 2.45 (s, 3H), 2.39-2.19 (m, 5H), 2.14-1.92 (m, 14H), 1.48-1.45 (m, 3H), 1.18-1.16 (m, 3H), 1.07-1.05 (m, 3H), 0.92-0.87 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)azetidine-1-carboxylate (Compound 126)

Step 1: Preparation of tert-butyl 3-[[5-(1-methoxycarbonyl-2-methyl-propyl)isoxazol-3-yl]oxyazetidine-1-carboxylate

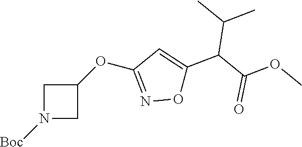 [SMILES: COC(=O)C(c1cc(OC2CN(C(=O)OC(C)(C)C)C2)no1)C(C)C]

To a stirred solution of methyl 2-(3-hydroxyisoxazol-5-yl)-3-methyl-butanoate (2.00 g, 10.0 mmol, 1.0 eq), PPh₃ (3.03 g, 11.6 mmol, 1.15 eq) and tert-butyl 3-hydroxyazetidine-1-carboxylate (1.91 g, 11.0 mmol, 1.1 eq) in THF (40 mL) was added DIAD (2.33 g, 11.6 mmol, 2.24 mL, 1.15 eq), the reaction mixture was stirred at 60° C. under N₂ for 16 h. The reaction was concentrated, the residue was purified by flash column chromatography (0˜5% tetrahydrofuran in petroleum ether) to give tert-butyl 3-[[5-(1-methoxycarbonyl-2-methyl-propyl)isoxazol-3-yl]oxyazetidine-1-carboxylate (3.45 g, 97%) as colorless gum. MS (ESI) m/z: 709.3 [M+H]⁺; ¹H NMR (CDCl₃, 400 MHz) δ 5.93 (s, 1H), 5.17-5.09 (m, 1H), 4.33-4.25 (m, 2H), 4.05-3.97 (m, 2H), 3.73 (s, 3H), 3.50 (d, J=8.4 Hz, 1H), 2.42-2.28 (m, 1H), 1.45 (s, 9H), 1.00 (d, J=6.4 Hz, 3H), 0.92 (d, J=6.8 Hz, 3H).

Step 2: Preparation of 2-[3-(1-tert-butoxycarbonylazetidin-3-yl)oxyisoxazol-5-yl]-3-methyl-butanoic acid

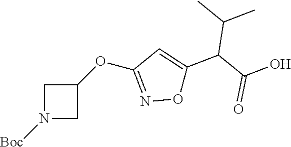 [SMILES: CC(C)C(C(=O)O)c1cc(OC2CN(C(=O)OC(C)(C)C)C2)no1]

To a stirred solution of tert-butyl 3-[[5-(1-methoxycarbonyl-2-methyl-propyl)isoxazol-3-yl]oxyazetidine-1-carboxylate (1.50 g, 4.2 mmol, 1.0 eq) in THF (5.0 mL) was added a solution of LiOH·H₂O (355 mg, 8.5 mmol, 2.0 eq) in water (5.0 mL), and the reaction mixture was stirred at 35° C. for 3 h. The pH of the reaction mixture was adjusted to 5 with 2 M aq. HCl and extracted with EtOAc (15 mL×3). The combined organic layer was washed with brine (20 mL), dried over anhydrous Na₂SO₄, filtered, and concentrated under reduced pressure to give 2-[3-(1-tert-butoxy carbonylazetidin-3-yl)oxyisoxazol-5-yl]-3-methyl-butanoic acid (1.40 g, 97%) as a white solid. MS (ESI) m/z: 681.2 [M+H]⁺.

Step 3: Preparation of tert-butyl 3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate

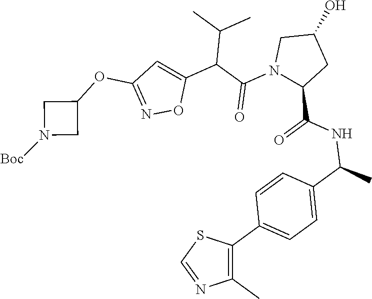 [SMILES: Cc1ncsc1-c1ccc([C@H](C)NC(=O)[C@@H]2C[C@@H](O)CN2C(=O)C(c2cc(OC3CN(C(=O)OC(C)(C)C)C3)no2)C(C)C)cc1]

To a stirred solution of 2-[3-(1-tert-butoxycarbonylazetidin-3-yl)oxyisoxazol-5-yl]-3-methyl-butanoic acid (1.40 g, 4.1 mmol, 1.0 eq) in CH₂Cl₂ (30 mL) were added DIEA (2.66 g, 20.6 mmol, 5.0 eq), HATU (1.88 g, 4.9 mmol, 1.2 eq) and (2S,4R)-4-hydroxy-N-[(1S)-1-[4-(4-methylthiazol-5-yl)phenyl]ethyl]pyrrolidine-2-carboxamide hydrochloride (1.96 g, 4.5 mmol, 1.1 eq), then the reaction mixture was stirred at 20° C. for 16 h. The reaction mixture was diluted with water (30 mL) and the layers were separated. The aqueous layer was extracted with CH₂Cl₂ (30 mL). The combined organic layer was washed with brine (30 mL), dried over anhydrous Na₂SO₄, filtered, and concentrated. The residue was purified by column chromatography (0˜90% ethyl acetate in petroleum ether) to give tert-butyl 3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (2.60 g, 97%) as a yellow solid. MS (ESI) m/z: 654.3 [M+H]⁺.

Step 4: Preparation of tert-butyl 3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate and tert-butyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate

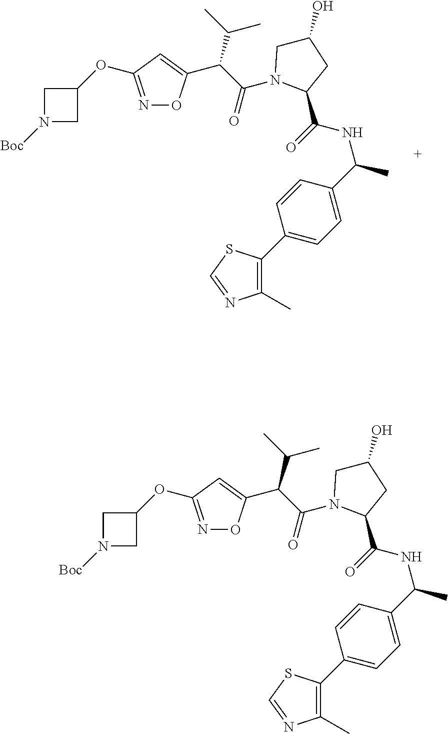 [SMILES: Cc1ncsc1-c1ccc([C@H](C)NC(=O)[C@@H]2C[C@@H](O)CN2C(=O)[C@H](c2cc(OC3CN(C(=O)OC(C)(C)C)C3)no2)C(C)C)cc1 ; Cc1ncsc1-c1ccc([C@H](C)NC(=O)[C@@H]2C[C@@H](O)CN2C(=O)[C@@H](c2cc(OC3CN(C(=O)OC(C)(C)C)C3)no2)C(C)C)cc1]

Tert-butyl 3-[[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (1.60 g) was separated by chiral SFC {column: DAICEL CHIRALPAK AD (250 mm*30 mm, 10 um); mobile phase: [0.1% NH₃H₂O EtOH]; B %: 40%}.

Tert-butyl 3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (811 mg) was obtained as a yellow solid. MS (ESI) m/z: 654.1 [M+H]⁺.

Tert-butyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (401 mg) was obtained as a yellow solid. MS (ESI) m/z: 654.1 [M+H]⁺.

Step 5: Preparation of tert-butyl 3-[7-[8-ethyl-3-(methoxymethoxy)-1-naphthyl]-8-fluoro-2-[[(3S,8S)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

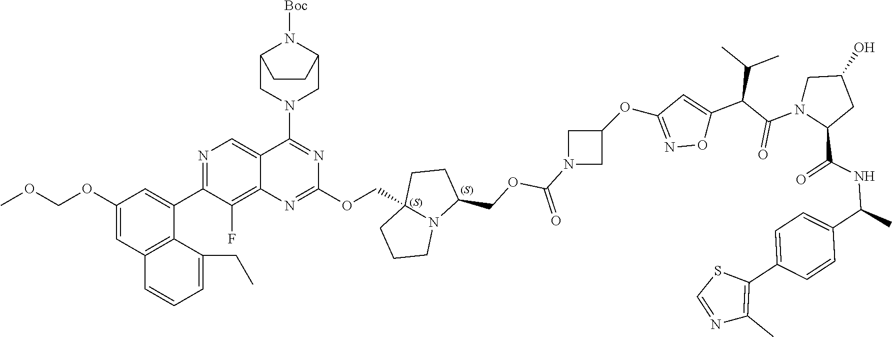

To a stirred solution of tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (150 mg, 0.202 mmol, 1.0 eq) in THF (4.0 mL) were added triethylamine (204 mg, 2.0 mmol, 10 eq), DMAP (2.5 mg, 0.02 mmol, 0.10 eq) and 4-nitrophenyl chloroformate (77 mg, 0.4 mmol, 1.9 eq), then the reaction mixture was stirred at 35° C. for 16 h. (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide trifluoroacetate (202 mg, 0.3 mmol, 1.5 eq) was added, and the reaction mixture was stirred at 35° C. for 2 h. The mixture was filtered and concentrated. The residue was purified by column chromatography (0˜6% methanol in chloromethane) to give tert-butyl 3-[7-[8-ethyl-3-(methoxymethoxy)-1-naphthyl]-8-fluoro-2-[[(3S,8S)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (239 mg, 90%) as a yellow solid. MS (ESI) m/z: 1322.3 [M+H]⁺.

Step 6: Preparation of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)azetidine-1-carboxylate

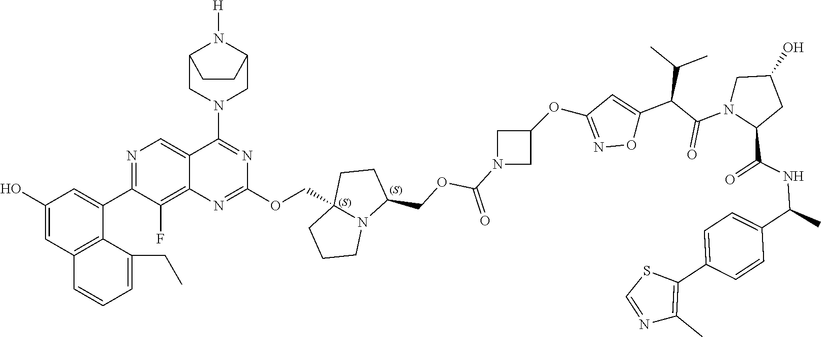

To a stirred solution of tert-butyl 3-[7-[8-ethyl-3-(methoxymethoxy)-1-naphthyl]-8-fluoro-2-[[(3S,8S)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (239 mg, 0.2 mmol, 1.0 eq) in CH₂Cl₂ (3.0 mL) was added 4 M HCl/dioxane (2.0 mL), and the reaction mixture was stirred at 20° C. for 10 min, bubbled with nitrogen to remove volatiles. The residue was dissolved in THF (30 mL) and treated with trimethylamine (0.30 mL). The suspension was stirred at 20° C. for 30 min and then filtered. The filtrate was concentrated under reduced pressure. The residue was purified by preparative HPLC (column: Phenomenex C18 75*30 mm*3 um; mobile phase: [water (FA)-MeCN]; B %: 5-45; 26 min) to give [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)azetidine-1-carboxylate (formate, 122.9 mg, 54%) as a white solid. MS (ESI) m/z: 1178.4 [M+H]⁺; ¹H NMR (CD₃OD, 400 MHz) δ 9.13-9.04 (m, 1H), 8.88 (s, 1H), 8.49 (s, 1H), 7.67-7.58 (m, 1H), 7.47-7.24 (m, 6H), 7.17 (d, J=7.2 Hz, 1H), 7.05-6.96 (m, 1H), 6.03 (s, 1H), 5.18-5.10 (m, 1H), 5.03 (q, J=6.8 Hz, 1H), 4.75-4.63 (m, 3H), 4.59-4.53 (m, 1H), 4.52-4.47 (m, 1H), 4.45-4.30 (m, 4H), 4.19-3.94 (m, 3H), 3.93-3.56 (m, 7H), 3.53-3.32 (m, 2H), 2.53-2.41 (m, 3H), 2.41-1.79 (m, 18H), 1.59-1.43 (m, 3H), 1.04 (d, J=6.4 Hz, 3H), 0.93-0.78 (m, 6H).

Exemplary Synthesis of [(3S,7aS)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)azetidine-1-carboxylate (Compound 127)

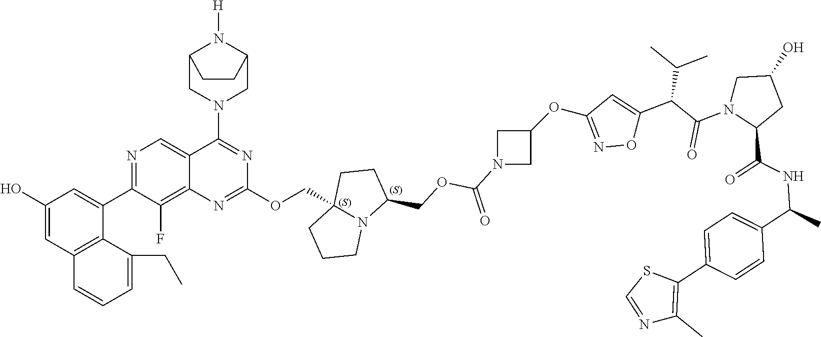

The title compound was prepared in an analogous manner to Compound 126 starting from (2S,4R)-1-[(2S)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formate, white solid). MS (ESI) m/z: 1178.4 [M+H]⁺; ¹H NMR (CD₃OD, 400 MHz) δ 9.14-9.07 (m, 1H), 8.92-8.83 (m, 1H), 8.47 (s, 1H), 7.63 (d, J=7.6 Hz, 1H), 7.50-7.27 (m, 6H), 7.20-7.12 (m, 1H), 7.03-6.97 (m, 1H), 6.06-6.00 (m, 1H), 5.13-5.05 (m, 1H), 5.02-4.95 (m, 1H), 4.79-4.64 (m, 3H), 4.61-4.53 (m, 2H), 4.51-4.25 (m, 5H), 4.22-4.13 (m, 1H), 3.96-3.74 (m, 6H), 3.73-3.61 (m, 2H), 3.58-3.49 (m, 1H), 3.38-3.33 (m, 1H), 2.51-2.45 (m, 3H), 2.44-1.79 (m, 18H), 1.58-1.43 (m, 3H), 1.07-0.92 (m, 3H), 0.92-0.79 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)azetidine-1-carboxylate (Compound 128)

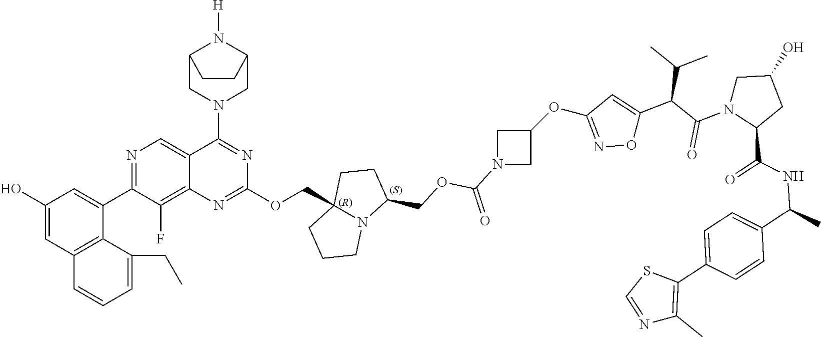

The title compound was prepared in an analogous manner to Compound 126 starting from (1R,5S)-tert-butyl 3-(7-(8-ethyl-3-(methoxymethoxy)naphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3,8-diazabicyclo[3.2.1]octane-8-carboxylate. (formate, white solid). MS (ESI) m/z: 1178.4 [M+H]⁺; ¹H NMR (CD₃OD, 400 MHz) δ 9.11 (d, J=5.2 Hz, 1H), 8.89-8.85 (m, 1H), 8.43 (s, 1H), 7.64 (d, J=8.0 Hz, 1H), 7.47-7.25 (m, 6H), 7.20-7.13 (m, 1H), 7.02 (dd, J=11.2, 2.4 Hz, 1H), 6.07-5.92 (m, 1H), 5.15-5.07 (m, 1H), 5.05-4.98 (m, 1H), 4.84-4.68 (m, 3H), 4.53-4.49 (m, 1H), 4.44-4.24 (m, 4H), 4.19-4.09 (m, 1H), 4.06-3.77 (m, 7H), 3.75-3.66 (m, 1H), 3.64-3.56 (m, 1H), 3.55-3.36 (m, 2H), 3.23-3.15 (m, 1H), 2.50-2.44 (m, 3H), 2.40-1.89 (m, 18H), 1.56-1.45 (m, 3H), 1.09-1.02 (m, 3H), 0.94-0.83 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 3-({5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)azetidine-1-carboxylate (Compound 129)

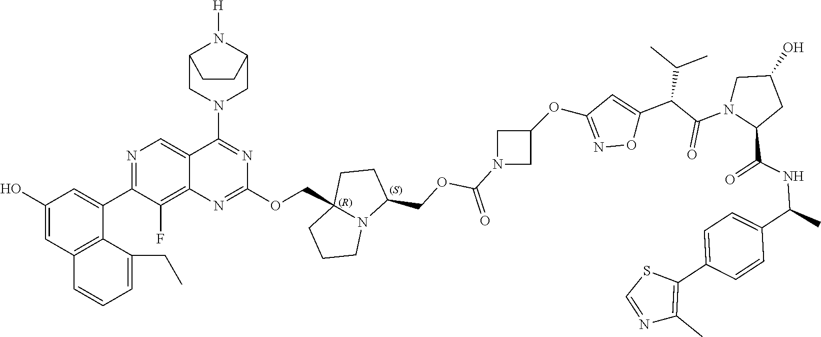

The title compound was prepared in an analogous manner to Compound 126 starting from (2S,4R)-1-[(2S)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formate, white solid). MS (ESI) m/z: 1178.3 [M+H]⁺; ¹H NMR (CD₃OD, 400 MHz) δ 9.08 (d, J=3.6 Hz, 1H), 8.89-8.82 (m, 1H), 8.49 (s, 1H), 7.62 (d, J=8.0 Hz, 1H), 7.50-7.24 (m, 6H), 7.15 (d, J=7.2 Hz, 1H), 7.04-6.97 (m, 1H), 6.08-5.98 (m, 1H), 5.13-5.03 (m, 1H), 4.99 (q, J=6.8 Hz, 1H), 4.81-4.68 (m, 2H), 4.57 (t, J=8.0 Hz, 1H), 4.49-4.20 (m, 6H), 4.14-3.75 (m, 8H), 3.73-3.62 (m, 2H), 3.43-3.34 (m, 1H), 3.15-3.06 (m, 1H), 2.50-2.42 (m, 3H), 2.42-2.18 (m, 5H), 2.17-1.71 (m, 13H), 1.59-1.43 (m, 3H), 1.08-0.93 (m, 3H), 0.92-0.81 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (3R)-4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-3-methylpiperazine-1-carboxylate (Compound 130)

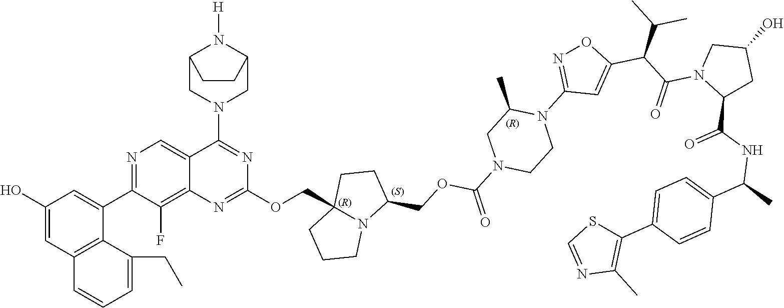

This compound was prepared in an analogous manner to Compound 120 starting from tert-butyl(3R)-methylpiperazine-1-carboxylate. (formate, white solid). MS (ESI) m/z: 1205.3 [M+H]⁺; ¹H NMR (CD₃OD, 400 MHz) δ 9.14 (s, 1H), 8.90-8.85 (m, 1H), 8.40 (s, 1H), 7.64 (d, J=8.0 Hz, 1H), 7.48-7.32 (m, 5H), 7.32-7.28 (m, 1H), 7.20-7.13 (m, 1H), 7.03 (t, J=2.8 Hz, 1H), 6.10-6.02 (m, 1H), 5.03 (q, J=6.8 Hz, 1H), 4.84-4.70 (m, 2H), 4.55-4.40 (m, 4H), 4.39-4.27 (m, 1H), 4.26-4.16 (m, 1H), 4.12-4.00 (m, 3H), 3.98-3.72 (m, 5H), 3.68-3.53 (m, 2H), 3.53-3.44 (m, 1H), 3.41-3.34 (m, 1H), 3.22-2.88 (m, 4H), 2.51-2.44 (m, 3H), 2.43-1.87 (m, 18H), 1.59-1.45 (m, 3H), 1.14-1.01 (m, 6H), 0.96-0.81 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (3R)-4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-3-methylpiperazine-1-carboxylate (Compound 131)

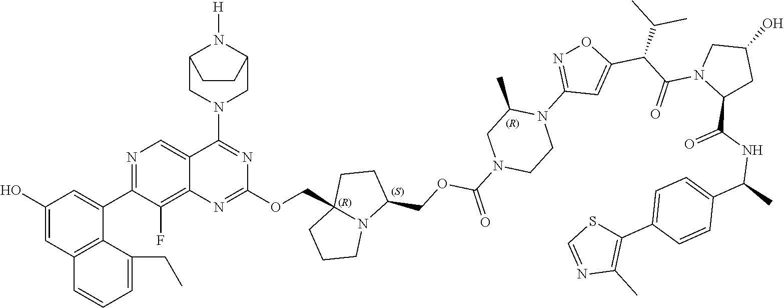

The title compound was prepared in an analogous manner to Compound 120 starting from tert-butyl(3R)-methylpiperazine-1-carboxylate. (formate, white solid). MS (ESI) m/z: 1205.3 [M+H]⁺; ¹H NMR (CD₃OD, 400 MHz) δ 9.10 (s, 1H), 8.91-8.83 (m, 1H), 8.45 (s, 1H), 7.63 (d, J=8.0 Hz, 1H), 7.51-7.25 (m, 6H), 7.16 (d, J=7.2 Hz, 1H), 7.02 (t, J=2.4 Hz, 1H), 6.13-6.04 (m, 1H), 5.15-4.96 (m, 1H), 4.82 (d, J=14.4 Hz, 1H), 4.73 (d, J=13.6 Hz, 1H), 4.59 (t, J=8.0 Hz, 1H), 4.55-4.40 (m, 3H), 4.39-4.24 (m, 1H), 4.22-4.12 (m, 1H), 4.11-3.97 (m, 3H), 3.96-3.58 (m, 7H), 3.52-3.42 (m, 1H), 3.39-3.33 (m, 1H), 3.21-2.82 (m, 4H), 2.51-2.43 (m, 3H), 2.43-1.84 (m, 18H), 1.61-1.42 (m, 3H), 1.12-0.94 (m, 6H), 0.93-0.81 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (2S)-4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2-methylpiperazine-1-carboxylate (Compound 132)

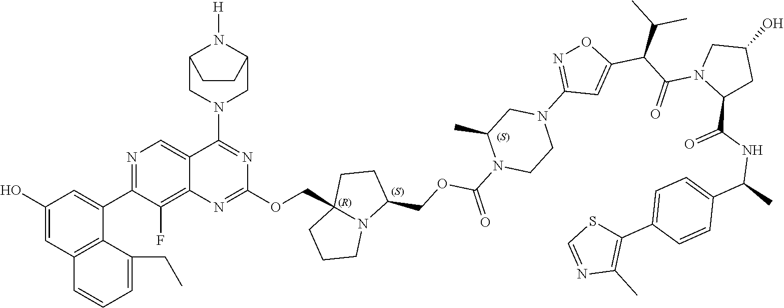

The title compound was prepared in an analogous manner to Compound 120 starting from tert-butyl(2S)-2-methylpiperazine-1-carboxylate. (formate, white solid). MS (ESI) m/z: 1205.9 [M+1]⁺; 1H NMR (400 MHz, DMSO-d₆) δ 9.13-8.94 (m, 2H), 8.46-8.35 (m, 1H), 8.20-8.16 (m, 1H), 7.72-7.61 (m, 1H), 7.45-7.26 (m, 5H), 7.17-7.06 (m, 1H), 7.02-6.91 (m, 1H), 6.22-6.03 (m, 1H), 4.91 (quint, J=7.0 Hz, 1H), 4.49-4.34 (m, 3H), 4.29-4.21 (m, 2H), 4.13-4.02 (m, 3H), 3.96 (dd, J=9.6, 5.6 Hz, 2H), 3.88-3.82 (m, 3H), 3.80 (d, J=1.6 Hz, 1H), 3.77-3.63 (m, 2H), 3.20-3.15 (m, 2H), 2.98-2.88 (m, 4H), 2.74-2.63 (m, 3H), 2.60-2.48 (m, 1H), 2.45 (s, 3H), 2.27-2.16 (m, 3H), 2.05-1.91 (m, 3H), 1.86-1.57 (m, 11H), 1.37 (d, J=7.2 Hz, 3H), 1.15 (d, J=6.8 Hz, 3H), 1.00-0.91 (m, 3H), 0.87-0.71 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl (2S)-4-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2-methylpiperazine-1-carboxylate (Compound 133)

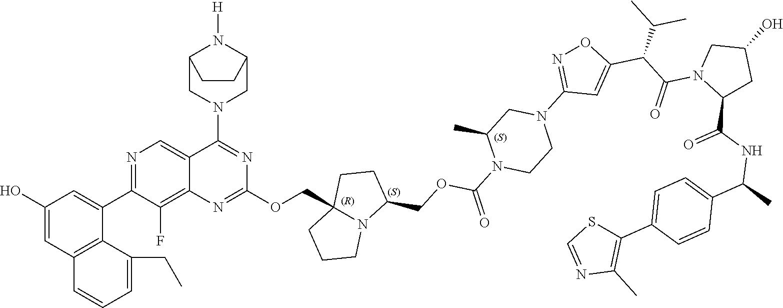

This compound was prepared in an analogous manner to Compound 120 starting from tert-butyl(2S)-2-methylpiperazine-1-carboxylate. (white solid). MS (ESI) m/z: 1206.0 [M+1]⁺; ¹H NMR (400 MHz, DMSO-d₆) δ 9.09 (s, 1H), 9.00-8.90 (m, 1H), 8.23 (d, J=7.6 Hz, 1H), 8.17 (s, 1H), 7.66 (d, J=8.4 Hz, 1H), 7.52-7.22 (m, 6H), 7.11 (d, J=7.2 Hz, 1H), 6.97 (t, J=2.8 Hz, 1H), 6.17 (s, 1H), 5.05-4.80 (m, 1H), 4.55-4.39 (m, 3H), 4.30-4.17 (m, 2H), 4.13-4.03 (m, 2H), 3.95 (dd, J=10.0, 5.2 Hz, 1H), 3.86-3.83 (m, 1H), 3.83-3.76 (m, 1H), 3.76-3.69 (m, 5H), 3.30 (d, J=9.6 Hz, 2H), 3.16-3.08 (m, 2H), 3.00-2.83 (m, 4H), 2.74-2.62 (m, 2H), 2.47-2.41 (m, 3H), 2.32-2.14 (m, 3H), 2.09-1.89 (m, 3H), 1.87-1.50 (m, 11H), 1.48-1.29 (m, 3H), 1.18-1.06 (m, 3H), 0.99-0.72 (m, 9H).

Exemplary Synthesis of (2S,4R)-1-[(2R*)-2-[3-(4-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperazin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 134)

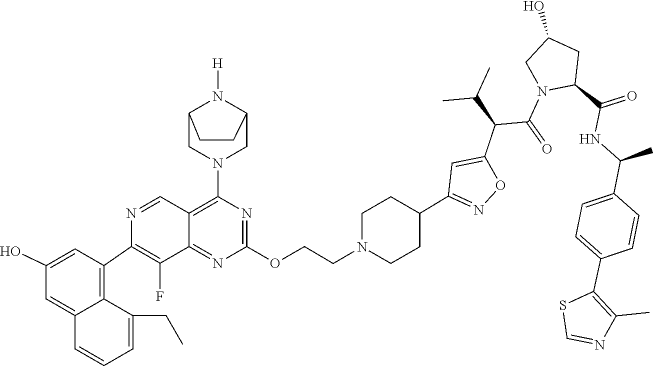 [SMILES: CCc1cccc2cc(O)cc(-c3ncc4c(N5CC6CCC(C5)N6)nc(OCCN5CCC(c6cc([C@H](C(=O)N7C[C@H](O)C[C@H]7C(=O)N[C@@H](C)c7ccc(-c8scnc8C)cc7)C(C)C)on6)CC5)nc4c3F)c12]

The title compound was prepared in an analogous manner to Compound 21 starting from (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (white solid). MS (ESI) m/z: 1038.4 [M+1]⁺; ¹H NMR (400 MHz, DMSO-d₆) δ 10.04-9.71 (m, 1H), 9.11 (s, 1H), 8.98 (s, 1H), 8.82-8.36 (m, 1H), 7.67 (br d, J=8.0 Hz, 1H), 7.49-7.41 (m, 2H), 7.40-7.32 (m, 3H), 7.31-7.26 (m, 1H), 7.19-7.09 (m, 1H), 7.00-6.92 (m, 1H), 6.20-5.98 (m, 1H), 5.19-4.98 (m, 1H), 4.97-4.86 (m, 1H), 4.63-4.46 (m, 4H), 4.39-4.23 (m, 2H), 3.96-3.88 (m, 2H), 3.80-3.67 (m, 4H), 3.59-3.53 (m, 2H), 3.18-3.13 (m, 4H), 2.80-2.72 (m, 2H), 2.62-2.54 (m, 4H), 2.45 (s, 3H), 2.28-2.12 (m, 3H), 2.06-1.97 (m, 1H), 1.90-1.72 (m, 5H), 1.48-1.32 (m, 3H), 1.01-0.91 (m, 3H), 0.88-0.73 (m, 6H).

Exemplary Synthesis of (2S,4R)-1-[(2R*)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)(methyl)amino]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 135)

Step 1: Preparation of tert-butyl 4-((5-(1-methoxy-3-methyl-1-oxobutan-2-yl)isoxazol-3-yl)(methyl)amino)piperidine-1-carboxylate

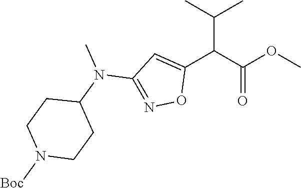 [SMILES: COC(=O)C(c1cc(N(C)C2CCN(C(=O)OC(C)(C)C)CC2)no1)C(C)C]

To a solution of tert-butyl 4-(methylamino)piperidine-1-carboxylate (3.56 g, 16.6 mmol, 1 eq) in N,N-dimethylacetamide (80 mL) was added methyl 3-methyl-2-(3-(((perfluorobutyl) sulfonyl)oxy)isoxazol-5-yl) butanoate (8 g, 16.6 mmol, 1 eq) and triethylamine (5.05 g, 49.9 mmol, 6.9 mL, 3 eq), the mixture was stirred at 140° C. for 4 h. Then 3-methyl-2-(3-(((perfluorobutyl) sulfonyl)oxy)isoxazol-5-yl) butanoate (8.00 g, 17 mmol, 1 eq) in N,N-dimethylacetamide (20 mL) was added, the mixture was stirred at 140° C. for 4 h. Diluted with water (100 mL) and extracted with ethyl acetate (100 mL×2). The combined organic layers were washed with brine (100 mL×2), dried over anhydrous sodium sulfate, filtered, and concentrated. The residue was purified by flash silica gel chromatography (0˜20% ethyl acetate/petroleum ether), then further purified by prep-HPLC (column: Phenomenex luna C18 150*40 mm*15 um; mobile phase: [water (trifluoroacetic acid)-acetonitrile]; B %: 44%-74%, 10 min) to afford tert-butyl 4-((5-(1-methoxy-3-methyl-1-oxobutan-2-yl)isoxazol-3-yl)(methyl)amino)piperidine-1-carboxylate (1.05 g, 70%) as a yellow oil. MS (ESI) m/z: 396.3 [M+1]⁺; 1H NMR (400 MHz, CDCl₃) δ 5.89 (s, 1H), 4.23 (br d, J=9.2 Hz, 2H), 3.74 (s, 3H), 3.73-3.65 (m, 1H), 3.48 (d, J=8.8 Hz, 1H), 2.84 (br s, 1H), 2.79 (s, 3H), 2.74 (br s, 1H), 2.35 (qd, J=6.8, 15.6 Hz, 1H), 1.80-1.70 (m, 2H), 1.69-1.56 (m, 2H), 1.48 (s, 9H), 1.01 (d, J=6.8 Hz, 3H), 0.94 (d, J=6.8 Hz, 3H).

Step 2: Preparation of 2-(3-((1-(tert-butoxycarbonyl) piperidin-4-yl)(methyl)amino)isoxazol-5-yl)-3-methylbutanoic acid

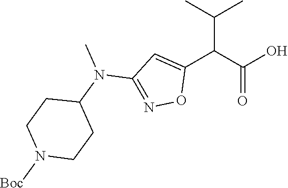 [SMILES: CC(C)C(C(=O)O)c1cc(N(C)C2CCN(C(=O)OC(C)(C)C)CC2)no1]

To a solution of tert-butyl 4-((5-(1-methoxy-3-methyl-1-oxobutan-2-yl)isoxazol-3-yl)(meth-yl)amino)piperidine-1-carboxylate (1.05 g, 2.7 mmol, 1 eq) in tetrahydrofuran (10 mL), methanol (10 mL) and water (20 mL) was added lithium hydroxide (318 mg, 13.3 mmol, 5 eq), the mixture was stirred at 20° C. for 2 h. The pH was adjusted to 6 by 1 M hydrochloric acid and the residue was diluted with water (30 mL) and extracted with ethyl acetate (30 mL×2). The combined organic layers were washed with brine (30 mL×2), dried over anhydrous sodium sulfate, filtered, and concentrated to afford 2-(3-((1-(tert-butoxycarbonyl) piperidin-4-yl)(methyl)amino)isoxazol-5-yl)-3-methylbutanoic acid (810 mg, 80%) as a yellow solid. MS (ESI) m/z: 382.3 [M+1]; 1H NMR (400 MHz, DMSO-d₆) δ 12.78 (br s, 1H), 6.17 (s, 1H), 4.08-3.97 (m, 2H), 3.73-3.58 (m, 1H), 3.39 (d, J=8.8 Hz, 1H), 3.32 (s, 3H), 2.86-2.70 (m, 2H), 2.24 (qd, J=6.8, 15.6 Hz, 1H), 1.62-1.48 (m, 4H), 1.40 (s, 9H), 0.96 (d, J=6.8 Hz, 3H), 0.83 (d, J=6.8 Hz, 3H).

Step 3: Preparation of tert-butyl 4-((5-(1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate

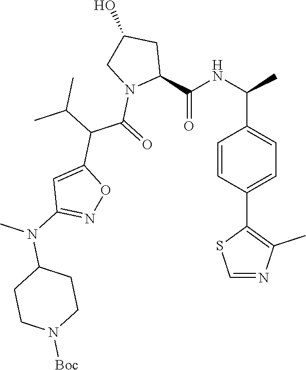

To a solution of (2S,4R)-4-hydroxy-N—((S)-1-(4-(4-methylthiazol-5-yl)phenyl)ethyl) pyrroled-ine-2-carboxamide hydrochloride (781 mg, 2.1 mmol, 1 eq) in N,N-dimethylformamide (10 mL) was added 2-(3-((1-(tert-butoxycarbonyl) piperidin-4-yl)(methyl)amino)isoxazol-5-yl)-3-methylbutanoic acid (810 mg, 2.12 mmol, 1 eq) and N,N-Diisopropylethylamine (1.37 g, 10.6 mmol, 1.8 mL, 5 eq), then O-(7-azabenzotriazol-1-yl)-N,N,N′,N′-tetramethyluronium hexafluorophosphate (1.29 g, 3.4 mmol, 1.6 eq) was added at 0° C., the mixture was stirred at 20° C. for 1 h. Then diluted with ethyl acetate (30 mL) and extracted with water (30 mL×2). The combined organic layers were washed with brine (30 mL×2), dried over anhydrous sodium sulfate, filtered, and concentrated. The residue was purified by prep-HPLC (column: Phenomenex luna C18 150*40 mm*15 um; mobile phase: [water (trifluoroacetic acid)-acetonitrile]; B %: 38%-68%, 10 min) to afford tert-butyl 4-((5-(1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate (970 mg, 64%) as an off-white solid. MS (ESI) m/z: 695.7 [M+1]⁺; 1H NMR (400 MHz, DMSO-d₆) δ 9.02-8.97 (m, 1H), 8.93-8.18 (m, 1H), 7.52-7.28 (m, 4H), 6.16-5.99 (m, 1H), 5.03-4.83 (m, 1H), 4.43-4.34 (m, 4H), 4.07-3.95 (m, 2H), 3.71-3.37 (m, 4H), 2.88-2.71 (m, 1H), 2.69-2.65 (m, 3H), 2.47-2.43 (m, 3H), 2.30-2.13 (m, 1H), 2.07-1.86 (m, 1H), 1.83-1.73 (m, 1H), 1.61-1.48 (m, 4H), 1.45 (br d, J=6.8 Hz, 1H), 1.39 (d, J=5.6 Hz, 9H), 1.38-1.32 (m, 2H), 0.95 (dd, J=4.0, 6.4 Hz, 2.5H), 0.85-0.75 (m, 3.5H).

Step 4: Preparation of tert-butyl 4-((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate and tert-butyl 4-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate

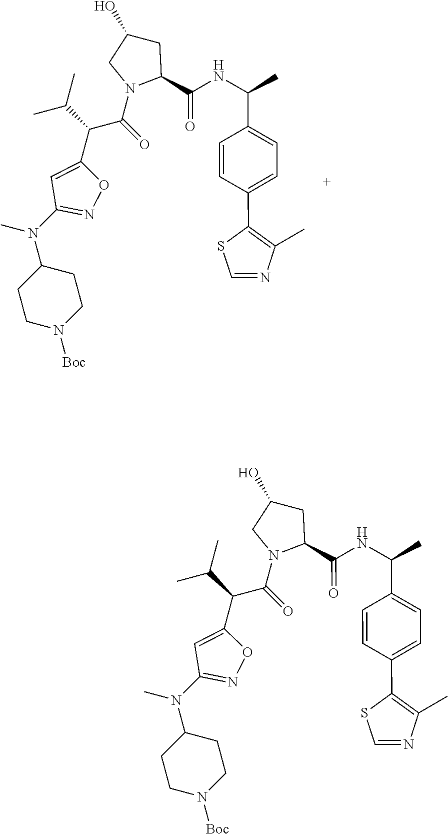

Tert-butyl 4-((5-(1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate (970 mg) was separated by SFC (column: DAICEL CHIRALPAK OD 250×30 mm, I.D., 10 um; mobile phase: isopropanol (0.1% NH₃H₂O) in CO₂ from 25% to 25%; flow rate: 70 mL/min; 220 nm).

Tert-butyl 4-((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate (370 mg, 38%) was obtained as a yellow solid.

Tert-butyl 4-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate (250 mg, 25%) was obtained as a yellow solid.

Step 5: Preparation of (2S,4R)-4-hydroxy-1-((R)-3-methyl-2-(3-(methyl(piperidin-4-yl)amino)isoxazol-5-yl)butanoyl)-N—((S)-1-(4-(4-methylthiazol-5-yl)phenyl)ethyl)pyrrolidine-2-carboxamide

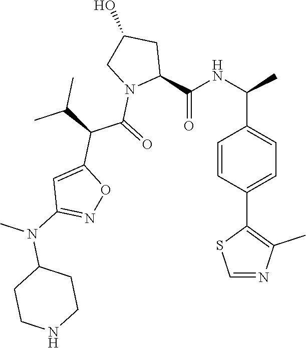

To a solution of tert-butyl 4-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate (120 mg, 0.2 mmol, 1 eq) in dichloromethane (2 mL) was added trifluoroacetic acid (1 mL), the mixture was stirred at 20° C. for 1 h, then concentrated to afford (2S,4R)-4-hydroxy-1-((R)-3-methyl-2-(3-(methyl(piperidin-4-yl)amino)isoxazol-5-yl)butanoyl)-N—((S)-1-(4-(4-methylthiazol-5-yl)phenyl)ethyl)pyrrolidine-2-carboxamide trifluoroacetate (121 mg, 98%) as a yellow oil. MS (ESI) m/z: 595.4 [M+1]⁺.

Step 6: Preparation of (1R,5S)-tert-butyl 3-(7-(8-ethyl-3-hydroxynaphthalen-1-yl)-8-fluoro-2-(2-(4-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino) piperidin-1-yl) ethoxy)pyrido[4,3-d]pyrimidin-4-yl)-3,8-diazabicyclo[3.2.1]octane-8-carboxylate

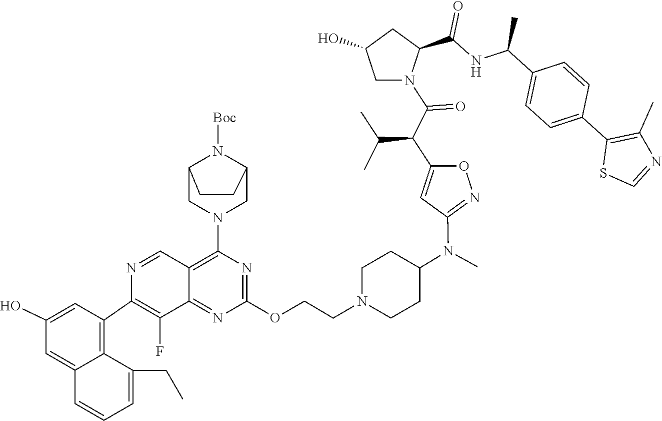 [SMILES: CCc1cccc2cc(O)cc(-c3ncc4c(N5CC6CCC(C5)N6C(=O)OC(C)(C)C)nc(OCCN5CCC(N(C)c6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of (2S,4R)-4-hydroxy-1-((R)-3-methyl-2-(3-(methyl(piperidin-4-isoxazol-5-yl)butanoyl)-N—((S)-1-(4-(4-methylthiazol-5-yl)amino) yl)phenyl)ethyl)pyrrolidine-2-carboxamide trifluoroacetate (120 mg, 0.2 mmol, 1 eq) in N,N-dimethyl formamide (3 mL) was added N-methylmorphine (52 mg, 0.5 mmol, 3 eq) for 10 min, then (1R,5S)-tert-butyl 3-(7-(8-ethyl-3-hydroxynaphthalen-1-yl)-8-fluoro-2-(2-oxoethoxy)pyrido[4,3-d]pyrimidin-4-yl)-3,8-diazabicyclo[3.2.1]octane-8-carboxylate (100 mg, 0.2 mmol, 1 eq) was added, the mixture was stirred at 20° C. for 20 min, sodium triacetoxyborohydride (36 mg, 0.2 mmol, 1 eq) was added, the mixture was stirred at 20° C. for 2 h. Diluted with water (30 mL) and extracted with dichloromethane (30 mL×2). The combined organic layers were washed with brine (30 mL×2), dried over anhydrous sodium sulfate, filtered, and concentrated. The residue was purified by preparative TLC (dichloromethane/methanol=10:1) to afford (1R,5S)-tert-butyl 3-(7-(8-ethyl-3-hydroxynaphthalen-1-yl)-8-fluoro-2-(2-(4-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino) piperidin-1-yl) ethoxy)pyrido[4,3-d]pyrimidin-4-yl)-3,8-diazabicyclo[3.2.1]octane-8-carboxylate (70 mg, 35%) as a white solid. MS (ESI) m/z: 584.0 [M/2+1]⁺.

Step 7: Preparation of (2S,4R)-1-[(2R*)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)(methyl)amino]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide

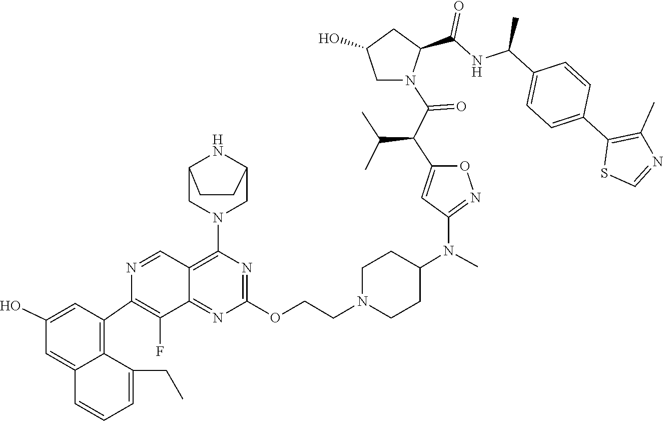 [SMILES: CCc1cccc2cc(O)cc(-c3ncc4c(N5CC6CCC(C5)N6)nc(OCCN5CCC(N(C)c6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of tert-butyl(1R,5S)-tert-butyl 3-(7-(8-ethyl-3-hydroxynaphthalen-1-yl)-8-fluoro-2-(2-(4-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino) piperidin-1-yl) ethoxy)pyrido[4,3-d]pyrimidin-4-yl)-3,8-diazabicyclo[3.2.1]octane-8-carboxylate (70 mg, 0.06 mmol, 1 eq) in dichloromethane (2 mL) was added trifluoroacetic acid (1 mL), the mixture was stirred at 20° C. for 1 h, then concentrated. The residue was purified by prep-HPLC (column: Unisil 3-100 C18 Ultra 150*50 mm*3 um; mobile phase: [water (formic acid)-acetonitrile]; B %: 12%-42%, 7 min) to (2S,4R)-1-[(2R*)-2-{3-[(1-{2-[(4-{3,8-diazabicyclo[3.2.1]octan-3-yl}-7-(8-ethyl-3-afford hydroxynaphthalen-1-yl)-8-fluoropyrido[4,3-d]pyrimidin-2-yl)oxy]ethyl}piperidin-4-yl)(methyl)amino]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (formate, 48.8 mg, 82%) as a white solid. MS (ESI) m/z: 534.1 [M/2+1]⁺; 1H NMR (400 MHz, DMSO-d₆) δ 9.12 (s, 1H), 8.99 (s, 1H), 8.83-8.37 (m, 1H), 8.18 (s, 1H), 7.68 (d, J=8.0 Hz, 1H), 7.48-7.41 (m, 2H), 7.41-7.33 (m, 3H), 7.29 (d, J=2.4 Hz, 1H), 7.13 (d, J=7.2 Hz, 1H), 6.98 (d, J=2.8 Hz, 1H), 6.13-5.93 (m, 1H), 4.99-4.63 (m, 2H), 4.58-4.46 (m, 4H), 4.37 (br t, J=7.6 Hz, 1H), 4.29 (br s, 1H), 3.84-3.75 (m, 3H), 3.72 (br dd, J=4.2, 10.4 Hz, 2H), 3.67-3.59 (m, 1H), 3.55 (br d, J=10.0 Hz, 1H), 3.51-3.40 (m, 3H), 3.04 (br d, J=10.4 Hz, 2H), 2.75 (br t, J=5.2 Hz, 2H), 2.69 (s, 3H), 2.46 (s, 3H), 2.32-2.25 (m, 1H), 2.24-2.20 (m, 1H), 2.19-2.12 (m, 3H), 2.06-1.98 (m, 1H), 1.81-1.75 (m, 4H), 1.74-1.68 (m, 2H), 1.57 (br d, J=8.4 Hz, 2H), 1.48-1.37 (m, 3H), 0.98-0.94 (m, 3H), 0.85-0.78 (m, 6H).

Exemplary Synthesis of (2S,4R)-1-[(2R*)-2-[3-(4-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperazin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 136)

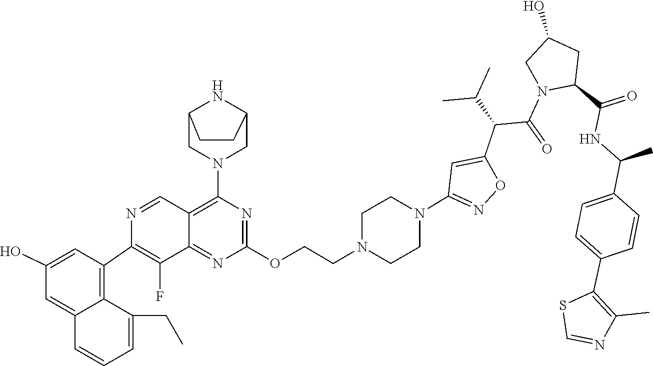 [SMILES: CCc1cccc2cc(O)cc(-c3ncc4c(N5CC6CCC(C5)N6)nc(OCCN5CCN(c6cc([C@@H](C(=O)N7C[C@H](O)C[C@H]7C(=O)N[C@@H](C)c7ccc(-c8scnc8C)cc7)C(C)C)on6)CC5)nc4c3F)c12]

The title compound was prepared in an analogous manner to Compound 21 starting from (2S,4R)-4-hydroxy-1-[(2S)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (white solid). MS (ESI) m/z: 1138.4 [M+1]⁺; 1H NMR (400 MHz, DMSO-d₆) δ 10.10-9.74 (m, 1H), 9.11 (s, 1H), 9.01-8.95 (m, 1H), 8.94-8.18 (m, 1H), 7.67 (d, J=8.4 Hz, 1H), 7.52-7.23 (m, 6H), 7.12 (d, J=7.2 Hz, 1H), 7.01-6.91 (m, 1H), 6.15 (s, 1H), 5.18-4.79 (m, 2H), 4.63-4.36 (m, 5H), 4.32-4.20 (m, 1H), 3.90-3.63 (m, 6H), 3.57-3.51 (m, 2H), 3.45-3.40 (m, 2H), 3.17-3.08 (m, 4H), 2.79-2.69 (m, 2H), 2.60-2.56 (m, 1H), 2.46-2.41 (m, 3H), 2.35-2.00 (m, 5H), 1.96-1.72 (m, 5H), 1.48-1.30 (m, 3H), 0.95 (d, J=6.4 Hz, 2H), 0.87-0.71 (m, 7H).

Exemplary Synthesis of (2S,4R)-1-[(2R*)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)(methyl)amino]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 137)

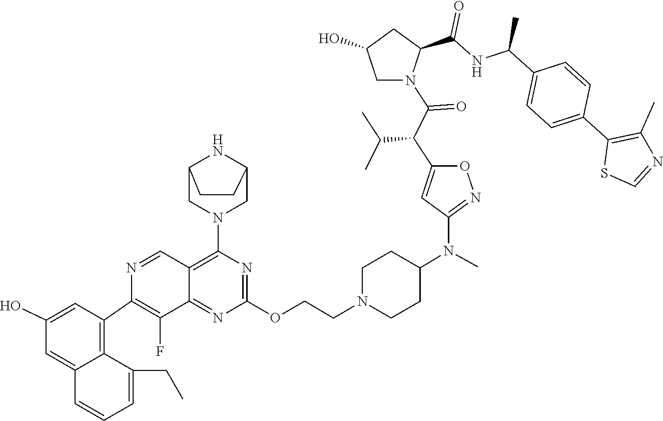 [SMILES: CCc1cccc2cc(O)cc(-c3ncc4c(N5CC6CCC(C5)N6)nc(OCCN5CCC(N(C)c6cc([C@@H](C(=O)N7C[C@H](O)C[C@H]7C(=O)N[C@@H](C)c7ccc(-c8scnc8C)cc7)C(C)C)on6)CC5)nc4c3F)c12]

The title compound was prepared in an analogous manner to Compound 135 starting from tert-butyl 4-((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)(methyl)amino)piperidine-1-carboxylate. (formate, white solid). MS (ESI) m/z: 534.1 [M/2+1]⁺; 1H NMR (400 MHz, DMSO-d₆) δ 9.12 (s, 1H), 9.03-8.95 (m, 1H), 8.94-8.21 (m, 1H), 8.17 (s, 1H), 7.68 (d, J=8.4 Hz, 1H), 7.50-7.42 (m, 1H), 7.42-7.37 (m, 2H), 7.36-7.30 (m, 2H), 7.29 (d, J=2.4 Hz, 1H), 7.13 (d, J=7.2 Hz, 1H), 6.97 (s, 1H), 6.18-6.02 (m, 1H), 5.04-4.80 (m, 1H), 4.58-4.40 (m, 5H), 4.27 (br s, 1H), 3.87-3.73 (m, 3H), 3.72-3.64 (m, 2H), 3.57-3.48 (m, 3H), 3.42 (br dd, J=4.0, 12.0 Hz, 3H), 3.08-2.94 (m, 2H), 2.81-2.71 (m, 2H), 2.70-2.65 (m, 3H), 2.48-2.42 (m, 3H), 2.29-2.24 (m, 1H), 2.24-2.18 (m, 1H), 2.18-2.06 (m, 2H), 2.06-1.86 (m, 2H), 1.83-1.74 (m, 4H), 1.71-1.62 (m, 2H), 1.61-1.50 (m, 2H), 1.48-1.33 (m, 3H), 1.00-0.73 (m, 9H).

Exemplary Synthesis of (2S,4R)-1-[(2R*)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)oxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 138)

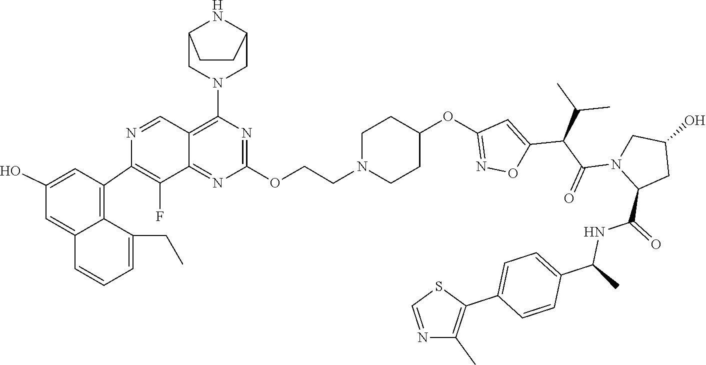 [SMILES: CCc1cccc2cc(O)cc(-c3ncc4c(N5CC6CCC(C5)N6)nc(OCCN5CCC(Oc6cc([C@H](C(=O)N7C[C@H](O)C[C@H]7C(=O)N[C@@H](C)c7ccc(-c8scnc8C)cc7)C(C)C)on6)CC5)nc4c3F)c12]

The title compound was prepared in an analogous manner to Compound 135 starting from tert-butyl 4-((5-((S)-1-((2S,4R)-4-hydroxy-2-(((R)-1-(4-(4-methylthiazol-5-yl)phenyl)ethyl)carbamoyl)pyrrolidin-1-yl)-3-methyl-1-oxobutan-2-yl)isoxazol-3-yl)oxy)piperidine-1-carboxylate. (formate, white solid). MS (ESI) m/z: 527.5 [M/2+1]⁺; 1H NMR (400 MHz, DMSO-d₆) δ 9.10 (s, 1H), 8.98 (s, 1H), 8.41 (d, J=7.6 Hz, 1H), 8.19 (s, 1H), 7.66 (d, J=8.0 Hz, 1H), 7.47-7.41 (m, 2H), 7.39-7.33 (m, 3H), 7.28 (d, J=2.8 Hz, 1H), 7.12 (d, J=7.2 Hz, 1H), 6.97 (d, J=2.8 Hz, 1H), 6.09-5.88 (m, 1H), 4.91 (quin, J=7.2 Hz, 1H), 4.54-4.43 (m, 5H), 4.37 (t, J=8.0 Hz, 1H), 4.28 (br s, 1H), 3.78-3.66 (m, 4H), 3.64 (br d, J=10.0 Hz, 2H), 3.45 (br d, J=11.6 Hz, 1H), 2.80 (br d, J=4.8 Hz, 2H), 2.73 (br t, J=5.6 Hz, 2H), 2.45 (s, 3H), 2.39-2.27 (m, 3H), 2.26-2.15 (m, 3H), 2.07-1.92 (m, 3H), 1.83-1.75 (m, 1H), 1.75-1.69 (m, 4H), 1.69-1.60 (m, 3H), 1.48-1.30 (m, 4H), 0.99-0.93 (m, 3H), 0.84-0.77 (m, 6H).

Exemplary Synthesis of (2S,4R)-1-[(2R*)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)oxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 139)

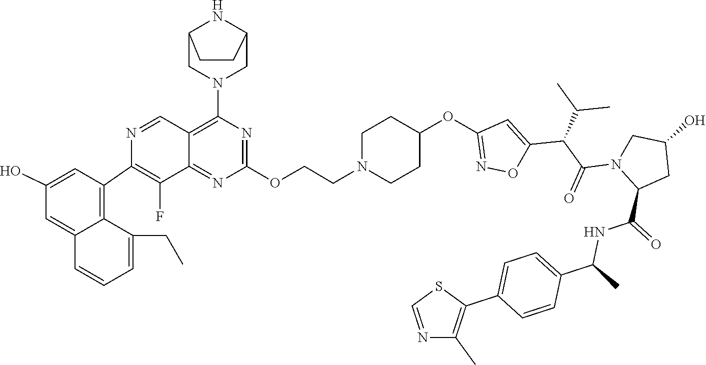 [SMILES: CCc1cccc2cc(O)cc(-c3ncc4c(N5CC6CCC(C5)N6)nc(OCCN5CCC(Oc6cc([C@@H](C(=O)N7C[C@H](O)C[C@H]7C(=O)N[C@@H](C)c7ccc(-c8scnc8C)cc7)C(C)C)on6)CC5)nc4c3F)c12]

The title compound was prepared in an analogous manner to Compound 135 starting from tert-butyl 4-((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)piperidine-1-carboxylate. (formate, white solid). MS (ESI) m/z: 527.5 [M/2+1]⁺; 1H NMR (400 MHz, DMSO-d₆) δ 9.10 (s, 1H), 9.00-8.95 (m, 1H), 8.28 (d, J=7.6 Hz, 1H), 8.21-8.13 (m, 1H), 7.66 (d, J=8.4 Hz, 1H), 7.50-7.41 (m, 1H), 7.41-7.29 (m, 4H), 7.28 (d, J=2.8 Hz, 1H), 7.11 (d, J=7.2 Hz, 1H), 6.97 (d, J=2.8 Hz, 1H), 6.13-6.06 (m, 1H), 4.94-4.83 (m, 1H), 4.54-4.46 (m, 4H), 4.46-4.39 (m, 3H), 4.26 (br d, J=2.8 Hz, 1H), 3.77-3.66 (m, 4H), 3.65-3.54 (m, 2H), 3.32 (br d, J=9.6 Hz, 1H), 2.79-2.72 (m, 2H), 2.71-2.66 (m, 2H), 2.44 (s, 3H), 2.31-2.22 (m, 4H), 2.22-2.13 (m, 2H), 2.08-1.91 (m, 3H), 1.82-1.74 (m, 1H), 1.70 (br s, 4H), 1.60 (br s, 2H), 1.49-1.28 (m, 4H), 0.96 (d, J=6.8 Hz, 2.5H), 0.85-0.78 (m, 6H), 0.75 (d, J=6.8 Hz, 0.5H).

Exemplary Synthesis of (2S,4R)-1-[(2R*)-2-[3-(7-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(1-methyl-1H-pyrazol-5-yl)phenyl]ethyl]pyrrolidine-2-carboxamide (Compound 140)

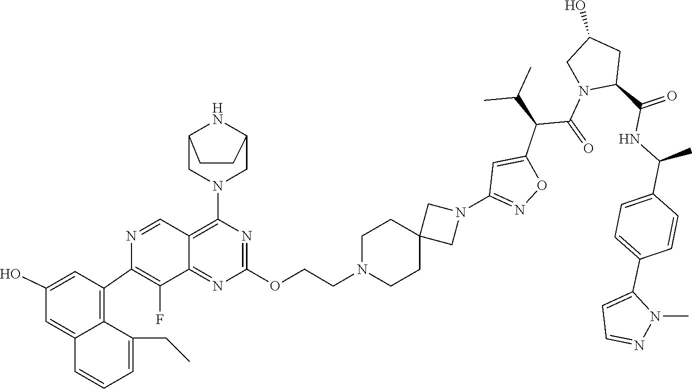 [SMILES: CCc1cccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8ccnn8C)cc7)C(C)C)on5)C6)nc4c3F)c12]

The title compound was prepared in an analogous manner to Compound 135 starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate. (formate, white solid). MS (ESI) m/z: 1061.6 [M+1]⁺; 1H NMR (400 MHz, DMSO-d₆) δ 9.09 (s, 1H), 8.41 (d, J=7.6 Hz, 1H), 8.16 (s, 1H), 7.67 (d, J=8.0 Hz, 1H), 7.50-7.43 (m, 3H), 7.40-7.33 (m, 3H), 7.28 (d, J=2.8 Hz, 1H), 7.12 (d, J=6.8 Hz, 1H), 6.96 (d, J=2.8 Hz, 1H), 6.36 (d, J=2.0 Hz, 1H), 5.81-5.71 (m, 1H), 4.96-4.88 (m, 1H), 4.52-4.41 (m, 4H), 4.38-4.33 (m, 1H), 4.31-4.25 (m, 1H), 3.84 (s, 3H), 3.72-3.60 (m, 6H), 3.58-3.52 (m, 5H), 3.46-3.36 (m, 3H), 2.72-2.65 (m, 2H), 2.44-2.36 (m, 4H), 2.30-2.13 (m, 4H), 2.07-1.96 (m, 1H), 1.82-1.76 (m, 1H), 1.74-1.67 (m, 7H), 1.47-1.35 (m, 3H), 0.97-0.91 (m, 3H), 0.84-0.75 (m, 6H).

Exemplary Synthesis of (2S,4R)-1-[(2R*)-2-[3-(7-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-2,7-diazaspiro[3.5]nonan-2-yl)-1,2-oxazol-5-yl]-3-methylbutanoyl]-4-hydroxy-N-[(1S)-1-[4-(1-methyl-1H-pyrazol-5-yl)phenyl]ethyl]pyrrolidine-2-carboxamide (Compound 141)

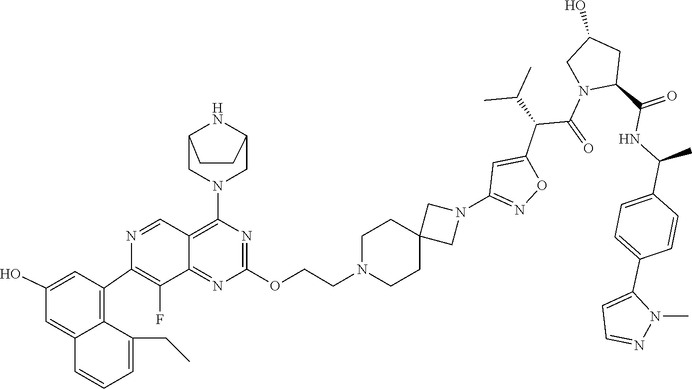 [SMILES: CCc1cccc2cc(O)cc(-c3ncc4c(N5CC6CCC(C5)N6)nc(OCCN5CCC6(CC5)CN(c5cc([C@@H](C(=O)N7C[C@H](O)C[C@H]7C(=O)N[C@@H](C)c7ccc(-c8ccnn8C)cc7)C(C)C)on5)C6)nc4c3F)c12]

The title compound was prepared in an analogous manner to Compound 135 starting from (2S,4R)-1-[(2S)-2-[3-(2,7-diazaspiro[3.5]nonan-2-yl)isoxazol-5-yl]-3-methyl-butanoyl]-4-hydroxy-N-[(1S)-1-[4-(2-methylpyrazol-3-yl)phenyl]ethyl]pyrrolidine-2-carboxamide. (white solid). MS (ESI) m/z: 1061.6 [M+1]⁺; 1H NMR (400 MHz, DMSO-d₆) δ 9.08 (s, 1H), 8.25-8.20 (m, 1H), 7.70-7.50 (m, 1H), 7.46-7.42 (m, 3H), 7.39-7.36 (m, 1H), 7.50-7.43 (m, 3H), 7.35-7.31 (m, 2H), 7.28 (d, J=2.8 Hz, 1H), 7.12 (d, J=6.8 Hz, 1H), 6.96 (d, J=2.8 Hz, 1H), 6.43-6.33 (m, 1H), 5.86 (s, 1H), 5.05-4.88 (m, 1H), 4.50-4.41 (m, 5H), 4.30-4.22 (m, 1H), 3.82-3.81 (m, 3H), 3.71-3.67 (m, 1H), 3.66-3.56 (m, 6H), 3.54-3.47 (m, 5H), 3.42-3.39 (m, 1H), 3.31-3.27 (m, 1H), 2.69-2.63 (m, 2H), 2.41-2.33 (m, 4H), 2.29-2.14 (m, 4H), 2.06-1.98 (m, 1H), 1.83-1.75 (m, 1H), 1.70-1.58 (m, 7H), 1.48-1.32 (m, 3H), 0.98-0.93 (m, 2H), 0.84-0.72 (m, 7H).

Exemplary Synthesis of (2S,4R*)-1-[(2S)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(1-methyl-1H-pyrazol-5-yl)phenyl]ethyl]pyrrolidine-2-carboxamide (Compound 142)

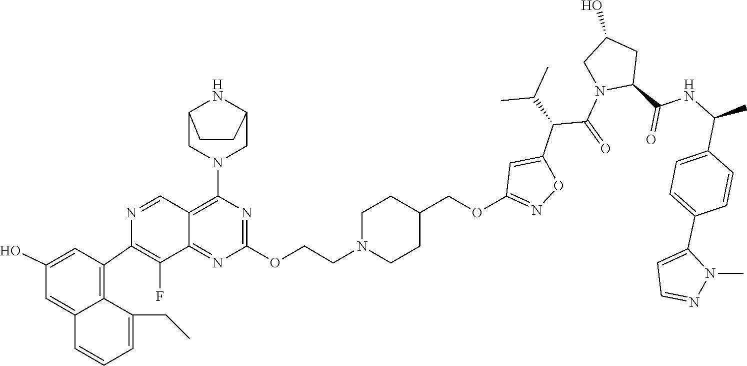 [SMILES: CCc1cccc2cc(O)cc(-c3ncc4c(N5CC6CCC(C5)N6)nc(OCCN5CCC(COc6cc([C@@H](C(=O)N7C[C@H](O)C[C@H]7C(=O)N[C@@H](C)c7ccc(-c8ccnn8C)cc7)C(C)C)on6)CC5)nc4c3F)c12]

The title compound was prepared in an analogous manner to Compound 135 starting from tert-butyl 4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate. (formate, yellow solid). MS (ESI) m/z: 1050.5 [M+1]+; 1H NMR (400 MHz, CDCl₃) δ 9.94 (s, 1H), 9.13 (s, 1H), 8.30 (d, J=8.0 Hz, 1H), 8.14 (s, 1H), 7.67 (d, J=8.4 Hz, 1H), 7.46-7.42 (m, 3H), 7.39-7.31 (m, 3H), 7.29 (d, J=2.4 Hz, 1H), 7.12 (d, J=7.2 Hz, 1H), 6.96 (d, J=2.4 Hz, 1H), 6.41-6.33 (m, 1H), 6.16-6.06 (m, 1H), 4.95-4.85 (m, 1H), 4.65-4.50 (m, 4H), 4.42 (d, J=8.0 Hz, 1H), 4.30-4.20 (m, 1H), 4.02-3.95 (m, 4H), 3.83 (s, 3H), 3.77-3.73 (m, 2H), 3.58-3.53 (m, 1H), 3.50-3.46 (m, 1H), 3.05-2.96 (m, 2H), 2.82-2.73 (m, 2H), 2.28-2.22 (m, 2H), 2.16-2.02 (m, 4H), 1.90-1.82 (m, 5H), 1.75-1.64 (m, 4H), 1.35 (d, J=7.2 Hz, 3H), 1.28-1.21 (m, 2H), 0.96 (d, J=6.8 Hz, 3H), 0.82 (d, J=6.8 Hz, 6H).

Exemplary Synthesis of (2S,4R)-1-[(2R*)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methoxy]-1,2-oxazol-5-yl}-3-methylbutanoyl]-4-hydroxy-N-[(1S)-1-[4-(1-methyl-1H-pyrazol-5-yl)phenyl]ethyl]pyrrolidine-2-carboxamide (Compound 143)

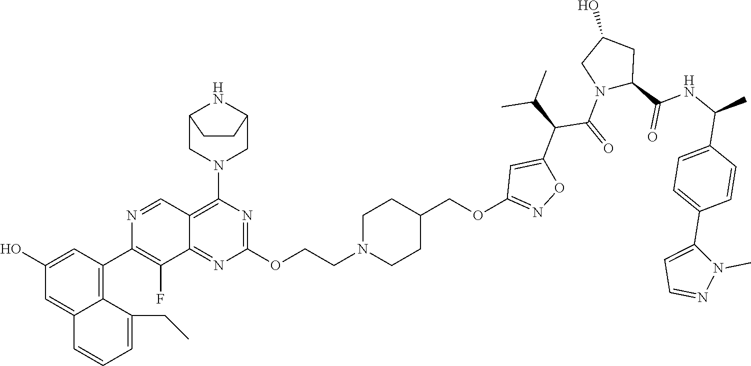 [SMILES: CCc1cccc2cc(O)cc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8ccnn8C)cc7)C(C)C)on6)CC5)nc4c3F)c12]

The title compound was prepared in an analogous manner to Compound 135 starting from tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]oxymethyl]piperidine-1-carboxylate. (formate, white solid). MS (ESI) m/z: 1050.5 [M]⁺; ¹H NMR (400 MHz, DMSO-d₆) δ 10.0 (s, 1H), 9.12 (s, 1H), 8.41 (d, J=7.6 Hz, 1H), 8.16 (s, 1H), 7.67 (d, J=8.0 Hz, 1H), 7.50-7.44 (m, 3H), 7.40-7.34 (m, 3H), 7.28 (d, J=2.4 Hz, 1H), 7.12 (d, J=7.2 Hz, 1H), 6.96 (d, J=2.8 Hz, 1H), 6.36 (d, J=2.0 Hz, 1H), 6.06 (s, 1H), 5.00-4.85 (m, 1H), 4.60-4.45 (m, 4H), 4.36 (d, J=8.0 Hz, 1H), 4.30-4.25 (m, 1H), 4.00-3.95 (m, 1H), 3.90-3.85 (m, 1H), 3.84 (s, 3H), 3.79-3.73 (m, 1H), 3.71-3.61 (m, 3H), 3.59-3.50 (m, 1H), 3.47-3.41 (m, 1H), 3.36-3.15 (m, 2H), 3.04-2.95 (m, 2H), 2.80-2.71 (m, 2H), 2.30-2.16 (m, 3H), 2.12-1.97 (m, 3H), 1.82-1.62 (m, 8H), 1.47-1.35 (m, 3H), 1.32-1.18 (m, 2H), 1.00-0.95 (m, 3H), 0.85-0.75 (m, 6H).

Exemplary Synthesis of [(3S,7aR)-7a-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-hexahydro-1H-pyrrolizin-3-yl]methyl 6-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-2,6-diazaspiro[3.3]heptane-2-carboxylate (Compound 144)

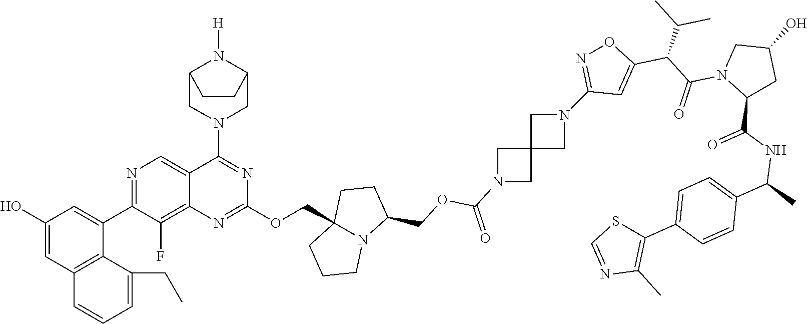 [SMILES: CCc1cccc2cc(O)cc(-c3ncc4c(N5CC6CCC(C5)N6)nc(OC[C@]56CCCN5[C@H](COC(=O)N5CC7(C5)CN(c5cc([C@@H](C(=O)N8C[C@H](O)C[C@H]8C(=O)N[C@@H](C)c8ccc(-c9scnc9C)cc8)C(C)C)on5)C7)CC6)nc4c3F)c12]

The title compound was prepared in an analogous manner to Compound 112 starting from tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and tert-butyl 6-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate. (formate, white solid). MS (ESI) m/z: 1203.5 [M+H]⁺; ¹H NMR (400 MHz, MeOD) δ 9.07-9.06 (s, 1H), 8.86 (s, 1H), 7.65-7.63 (m, 1H), 7.47-7.35 (m, 5H), 7.31-7.29 (m, 1H), 7.18-7.16 (m, 1H), 7.03-7.02 (m, 1H), 5.87-5.86 (m, 1H), 5.02-4.97 (m, 1H), 4.66-4.56 (m, 6H), 4.48-4.38 (m, 2H), 4.23-4.17 (m, 1H), 4.08-3.92 (m, 10H), 3.85-3.62 (m, 5H), 3.14-3.06 (m, 1H), 2.46 (s, 3H), 2.38-2.19 (m, 6H), 2.09-1.87 (m, 12H), 1.48-1.46 (m, 3H), 1.06-1.04 (m, 3H), 0.90-0.87 (m, 6H).

Exemplary Synthesis of [(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]-2,6-diazaspiro[3.3]heptane-2-carboxylate (Compound 340)

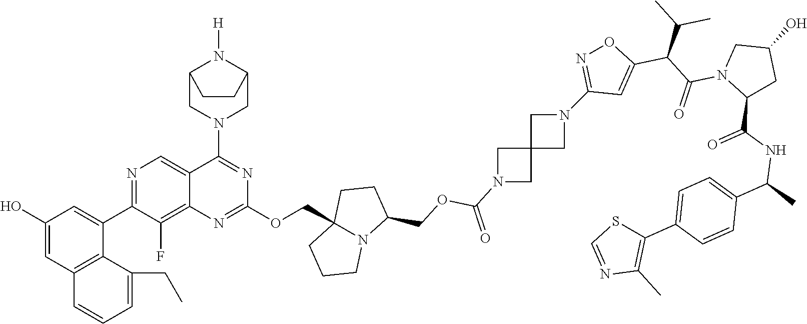 [SMILES: CCc1cccc2cc(O)cc(-c3ncc4c(N5CC6CCC(C5)N6)nc(OC[C@]56CCCN5[C@H](COC(=O)N5CC7(C5)CN(c5cc([C@H](C(=O)N8C[C@H](O)C[C@H]8C(=O)N[C@@H](C)c8ccc(-c9scnc9C)cc8)C(C)C)on5)C7)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from (2S,4R)-1-[(2R)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate. (light yellow solid). LC/MS (ESI) m/z: 1203.4 [M+H]⁺. ¹H NMR (400 MHz, CD₃OD) δ 9.03 (s, 1H), 8.88 (s, 1H), 7.61-7.59 (m, 1H), 7.45-7.31 (m, 5H), 7.26-7.25 (m, 1H), 7.13-7.11 (m, 1H), 7.02-7.01 (m, 1H), 5.86-5.83 (m, 1H), 5.05-4.99 (m, 1H), 4.66-4.57 (m, 2H), 4.52-4.48 (m, 1H), 4.44 (s, 1H), 4.29-4.15 (m, 6H), 4.06-3.92 (m, 6H), 3.83-3.78 (m, 1H), 3.73-3.54 (m, 6H), 3.05-2.99 (m, 2H), 2.48 (s, 3H), 2.35-2.17 (m, 6H), 1.95-1.77 (m, 12H), 1.52-1.50 (m, 3H), 1.04-1.03 (m, 3H), 0.91-0.86 (m, 6H).

Exemplary Synthesis of [(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2R,5S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (Compound 302)

Step 1: Preparation of tert-butyl(2R,5S)-4-[5-(1-methoxycarbonyl-2-methyl-propyl)isoxazol-3-yl]-2,5-dimethyl-piperazine-1-carboxylate

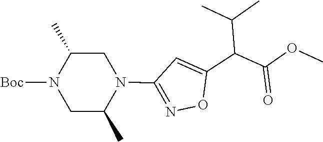 [SMILES: COC(=O)C(c1cc(N2C[C@@H](C)N(C(=O)OC(C)(C)C)C[C@@H]2C)no1)C(C)C]

To a solution of methyl 3-methyl-2-[3-(1,1,2,2,3,3,4,4,4-nonafluorobutylsulfonyloxy)isoxazol-5-yl]butanoate (5.00 g, 10.39 mmol, 1.0 eq) in DMA (60 mL) were added tert-butyl(2R,5S)-2,5-dimethylpiperazine-1-carboxylate (4.45 g, 20.78 mmol, 2.0 eq) and triethylamine (2.9 mL, 20.78 mmol, 2.0 eq), and the reaction mixture was stirred at 145° C. for 5 hours. The mixture was diluted with water (100 mL) and extracted with EtOAc (3×100 mL). The combined organic extracts were washed with brine (2×100 mL), dried over anhydrous Na₂SO₄, filtered, and concentrated under reduced pressure. The residue was purified by flash chromatography on SiO₂ (gradient: 0˜10% EtOAc) to afford tert-butyl(2R,5S)-4-[5-(1-methoxycarbonyl-2-methyl-propyl)isoxazol-3-yl]-2,5-dimethyl-piperazine-1-carboxylate (1.6 g, 3.45 mmol, 33.25% yield) as a white solid. LC/MS (ESI) m/z: 396.0 [M+H]⁺. ¹H NMR (400 MHz, CDCl₃) δ 5.91 (s, 1H), 4.53-4.24 (m, 1H), 3.85 (d, J=3.6 Hz, 1H), 3.74 (s, 3H), 3.49 (d, J=8.4 Hz, 1H), 3.43-3.19 (m, 3H), 2.44-2.25 (m, 1H), 2.05 (s, 1H), 1.48 (s, 9H), 1.24 (d, J=7.2 Hz, 3H), 1.15 (d, J=6.4 Hz, 3H), 1.01 (d, J=6.4 Hz, 3H), 0.94 (d, J=6.8 Hz, 3H).

Step 2: Preparation of 2-[3-[(2S,5R)-4-tert-butoxycarbonyl-2,5-dimethyl-piperazin-1-yl]isoxazol-5-yl]-3-methyl-butanoic acid

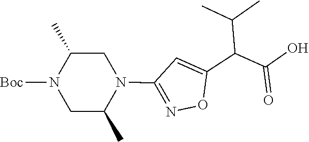 [SMILES: CC(C)C(C(=O)O)c1cc(N2C[C@@H](C)N(C(=O)OC(C)(C)C)C[C@@H]2C)no1]

To a solution of tert-butyl(2R,5S)-4-[5-(1-methoxycarbonyl-2-methyl-propyl)isoxazol-3-yl]-2,5-dimethyl-piperazine-1-carboxylate (1.6 g, 4.05 mmol, 1.0 eq) in THF (15 mL) and H₂O (5 mL) was added LiOH·H₂O (848.0 mg, 20.23 mmol, 5.0 eq), and the reaction mixture was stirred at 25° C. for 10 hours. The mixture was concentrated, diluted with water (20 mL), and the pH adjusted until pH ˜4 with by addition of 2N HCl. The resulting mixture was extracted with CH₂Cl₂ (70 mL×3), and the combined organic extract was washed with brine (15 mL×3), dried over anhydrous Na₂SO₄, filtered, and concentrated under reduced pressure to afford 2-[3-[(2S,5R)-4-tert-butoxycarbonyl-2,5-dimethyl-piperazin-1-yl]isoxazol-5-yl]-3-methyl-butanoic acid (1.46 g, crude) a white solid. LC/MS (ESI) m/z: 382.0 [M+H]⁺.

Step 3: Preparation of tert-butyl(2R,5S)-4-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate

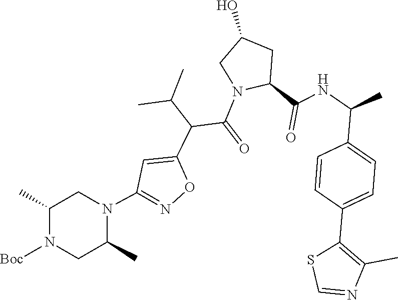

To a solution of (2S,4R)-4-hydroxy-N-[(1S)-1-[4-(4-methylthiazol-5-yl)phenyl]ethyl]pyrrolidine-2-carboxamide (2.2 g HCl salt, 4.21 mmol, 70% purity, 1.1 eq) in CH₂Cl₂ (25 mL) were added DIEA (6.6 mL, 38.27 mmol, 10.0 eq), 2-[3-[(2S,5R)-4-tert-butoxycarbonyl-2,5-dimethyl-piperazin-1-yl]isoxazol-5-yl]-3-methyl-butanoic acid (1.46 g, 3.83 mmol, 1.0 eq), and HATU (1.60 g, 4.21 mmol, 1.1 eq), and the reaction mixture was stirred at 25° C. for 30 minutes. The reaction mixture was diluted with water (100 mL) and extracted with CH₂Cl₂ (100 mL×3). The combined extracts were washed with brine (100 mL×2), dried over anhydrous Na₂SO₄, filtered, and concentrated under reduced pressure. The residue was purified by flash chromatography on SiO₂ (gradient: 0˜10% THF in petroleum ether) to afford tert-butyl(2R,5S)-4-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (2 g, 2.28 mmol, 60% yield) as a white solid. LC/MS (ESI) m/z: 695.1 [M+H]⁺.

Step 4: Preparation of tert-butyl(2R,5S)-4-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate

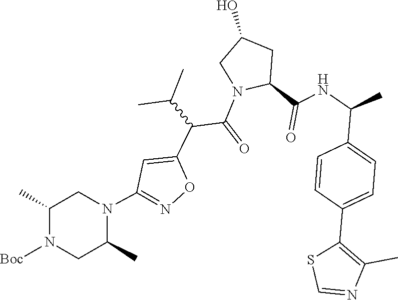

To a solution of tert-butyl(2R,5S)-4-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (1.00 g, 1.44 mmol, 1.0 eq) in dioxane (10 mL) was added Cs₂CO₃ (469.0 mg, 1.44 mmol, 1.0 eq), and the reaction mixture was stirred at 75° C. for 10 hours. The mixture was filtered through celite pad, and the filtrate was evaporated to afford the tert-butyl(2R,5S)-4-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (1.0 g) as a yellow solid. LC/MS (ESI) m/z: 695.1 [M+H]⁺.

Step 5: Preparation of tert-butyl(2R,5S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate & tert-butyl(2R,5S)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate

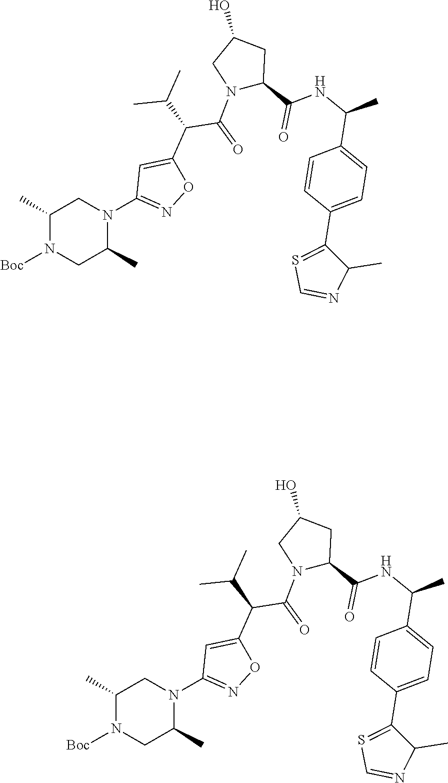

The diastereomers were separated by chiral SFC {column: IA 250 mm*50 mm*10 um; mobile phase: [0.1% NH₃H₂O IPA]; B %: 45%-45%, Gradient time: 60 min}. Fraction 1 was concentrated under reduced pressure to afford tert-butyl(2R,5S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (620 mg, 0.892 mmol) as a white solid. LC/MS (ESI) m/z: 695.1 [M+H]⁺.

Fraction 2 was concentrated under reduced pressure to afford tert-butyl(2R,5S)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (337 mg, 0.485 mmol) as a white solid. LC/MS (ESI) m/z: 695.1 [M+H]⁺.

Step 6: Preparation of (2S,4R)-1-[(2S)-2-[3-[(2S,5R)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

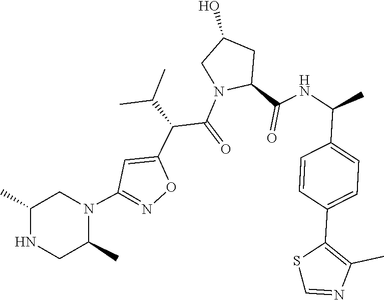

To a solution of tert-butyl(2R,5S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (400 mg, 0.576 mmol, 1.0 eq) in CH₂Cl₂ (2.0 mL) was added 4 M HCl/dioxane (2.0 mL) dropwise, and the reaction mixture was stirred at 25° C. for 1 hour. The mixture was concentrated to afford (2S,4R)-1-[(2S)-2-[3-[(2S,5R)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (360 mg, 0.570 mmol, HCl salt) as a yellow solid. LC/MS (ESI) m/z: 595.2 [M+H]⁺.

Step 7: Preparation of [(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2R,5S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate

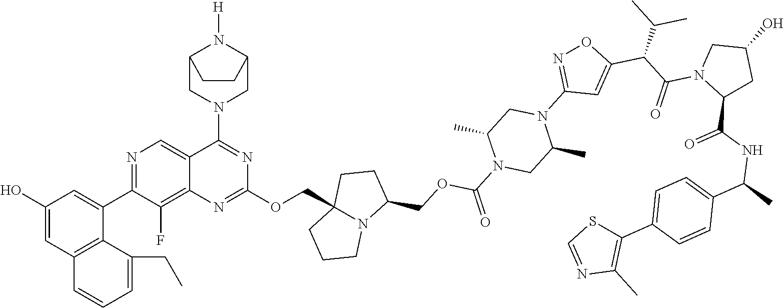 [SMILES: CCc1cccc2cc(O)cc(-c3ncc4c(N5CC6CCC(C5)N6)nc(OC[C@]56CCCN5[C@H](COC(=O)N5C[C@H](C)N(c7cc([C@@H](C(=O)N8C[C@H](O)C[C@H]8C(=O)N[C@@H](C)c8ccc(-c9scnc9C)cc8)C(C)C)on7)C[C@H]5C)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-1-[(2S)-2-[3-[(2S,5R)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (HCl salt). (formic acid salt, white solid). LC/MS (ESI) m/z: 1093.2 [M+H]⁺. ¹H NMR (400 MHz, CD₃OD) δ 9.14-9.09 (m, 1H), 8.90-8.85 (m, 1H), 7.63-7.60 (m, 1H), 7.47-7.33 (m, 5H), 7.29 (d, J=2.4 Hz, 1H), 7.19-7.13 (m, 1H), 7.01 (s, 1H), 6.15-6.05 (m, 1H), 4.99-4.96 (m, 1H), 4.83-4.77 (m, 3H), 4.63-4.06 (m, 10H), 4.00-3.57 (m, 8H), 3.53-3.33 (m, 3H), 3.24-3.09 (m, 3H), 2.50-2.44 (m, 3H), 2.40-2.26 (m, 4H), 2.09-1.93 (m, 10H), 1.58-1.45 (m, 3H), 1.21-1.13 (m, 3H), 1.09-0.99 (m, 6H), 0.94-0.85 (m, 6H).

Exemplary Synthesis of Preparation of [(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2R,5S)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (Compound 296)

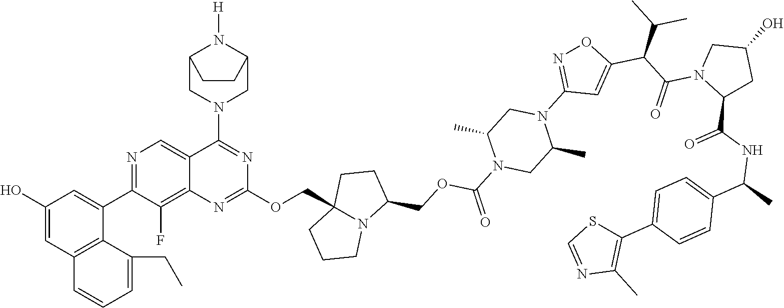 [SMILES: CCc1cccc2cc(O)cc(-c3ncc4c(N5CC6CCC(C5)N6)nc(OC[C@]56CCCN5[C@H](COC(=O)N5C[C@H](C)N(c7cc([C@H](C(=O)N8C[C@H](O)C[C@H]8C(=O)N[C@@H](C)c8ccc(-c9scnc9C)cc8)C(C)C)on7)C[C@H]5C)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from tert-butyl 3-[7-[8-ethyl-3-(methoxymethoxy)-1-naphthyl]-8-fluoro-2-[[(3S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-1-[(2R)-2-[3-[(2S,5R)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (HCl salt). (formic acid salt, white solid). LC/MS (ESI) m/z: 1220.1 [M+H]⁺. ¹H NMR (400 MHz, CD₃OD) δ 9.14-9.04 (m, 1H), 8.88 (s, 1H), 8.49 (s, 1H), 7.63 (d, J=7.2 Hz, 1H), 7.49-7.31 (m, 5H), 7.31-7.28 (m, 1H), 7.16 (dd, J=3.6, 6.4 Hz, 1H), 7.03 (s, 1H), 6.07 (s, 1H), 5.07-4.99 (m, 1H), 4.82-4.12 (m, 13H), 3.93-3.72 (m, 7H), 3.67-3.56 (m, 2H), 3.50-3.40 (m, 1H), 3.24-2.94 (m, 4H), 2.50-2.45 (m, 3H), 2.40-2.25 (m, 4H), 2.04-1.83 (m, 10H), 1.60-1.46 (m, 3H), 1.27-1.18 (m, 3H), 1.12-1.01 (m, 6H), 0.94-0.84 (m, 6H).

Exemplary Synthesis of [(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2S,5R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (Compound 297)

Step 1: Preparation of (2S,4R)-1-[(2S)-2-[3-[(2R,5S)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

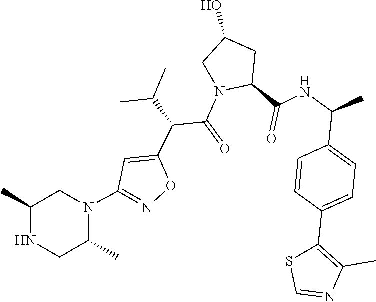

The title compound was prepared in an analogous manner to (2S,4R)-1-[(2S)-2-[3-[(2S,5R)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl(2S,5R)-2,5-dimethylpiperazine-1-carboxylate. (HCl salt, yellow solid). LC/MS (ESI) m/z: 595.0 [M+H]⁺.

Step 2: Preparation of [(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2S,5R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate

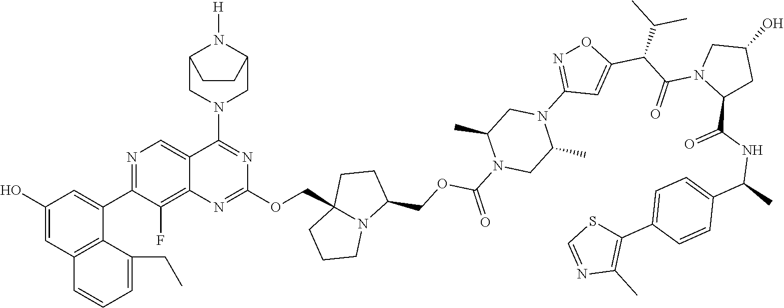 [SMILES: CCc1cccc2cc(O)cc(-c3ncc4c(N5CC6CCC(C5)N6)nc(OC[C@]56CCCN5[C@H](COC(=O)N5C[C@@H](C)N(c7cc([C@@H](C(=O)N8C[C@H](O)C[C@H]8C(=O)N[C@@H](C)c8ccc(-c9scnc9C)cc8)C(C)C)on7)C[C@@H]5C)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from tert-butyl 3-[7-[8-ethyl-3-(methoxymethoxy)-1-naphthyl]-8-fluoro-2-[[(3S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-1-[(2S)-2-[3-[(2R,5S)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (HCl salt). (formic acid salt, white solid). LC/MS (ESI) m/z: 1220.0 [M+H]⁺. ¹H NMR (400 MHz, CD₃OD) δ 9.07 (s, 1H), 8.87 (s, 1H), 8.49 (s, 1H), 7.63 (d, J=8.4 Hz, 1H), 7.48-7.34 (m, 5H), 7.30 (d, J=2.0 Hz, 1H), 7.16 (d, J=6.4 Hz, 1H), 7.03-7.01 (m, 1H), 6.11 (s, 1H), 4.99 (s, 1H), 4.64-4.56 (m, 6H), 4.46-4.33 (m, 4H), 4.31-3.99 (m, 3H), 3.95-3.49 (m, 11H), 3.15-2.88 (m, 2H), 2.49-2.43 (m, 3H), 2.41-2.22 (m, 5H), 2.05-1.86 (m, 10H), 1.48 (d, J=7.2 Hz, 3H), 1.23-1.15 (m, 3H), 1.06 (d, J=6.4 Hz, 6H), 0.94-0.86 (m, 6H).

Exemplary Synthesis of [(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2S,5R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,5-dimethyl-piperazine-1-carboxylate (Compound 299)

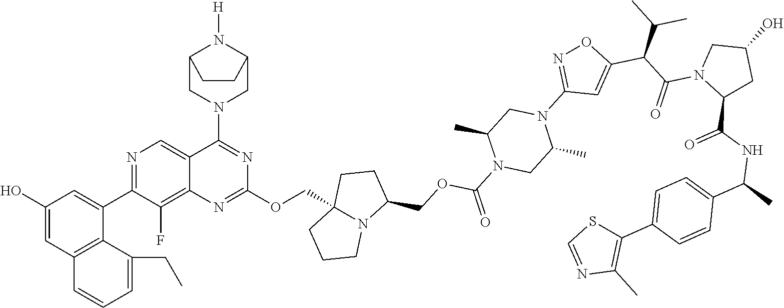 [SMILES: CCc1cccc2cc(O)cc(-c3ncc4c(N5CC6CCC(C5)N6)nc(OC[C@@]56CCCN5[C@H](COC(=O)N5C[C@@H](C)N(c7cc([C@H](C(=O)N8C[C@H](O)C[C@H]8C(=O)N[C@@H](C)c8ccc(-c9scnc9C)cc8)C(C)C)on7)C[C@@H]5C)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-1-[(2R)-2-[3-[(2R,5S)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (HCl salt). (formic acid salt, light yellow solid). LC/MS (ESI) m/z: 1220.1 [M+H]⁺. ¹H NMR (400 MHz, CD₃OD) δ 9.16-9.11 (m, 1H), 8.88 (s, 1H), 7.65-7.63 (m, 1H), 7.43-7.31 (m, 6H), 7.17-7.16 (m, 1H), 7.03-7.02 (m, 1H), 6.07 (s, 1H), 5.04-5.01 (m, 1H), 4.52-4.44 (m, 6H), 4.31-4.16 (m, 4H), 4.06-3.94 (m, 3H), 3.91-3.35 (m, 11H), 3.13-3.12 (m, 2H), 2.48 (s, 3H), 2.38-2.24 (m, 4H), 2.09-1.93 (m, 11H), 1.56-1.50 (m, 3H), 1.21 (s, 3H), 1.10-1.02 (m, 6H), 0.93-0.87 (m, 6H).

Exemplary Synthesis of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (3R)-4-[5-[(1S)-1-[(2S,4R)-4-hydroxy-2-[[(1S)-1-[4-(4-methyl thiazol-5-yl)phenyl]ethyl]carbamoyl]pyrrolidine-1-carbonyl]-isobutyl]isoxazol-3-yl]-3-methyl-piperazine-1-carboxylate (Compound 341)

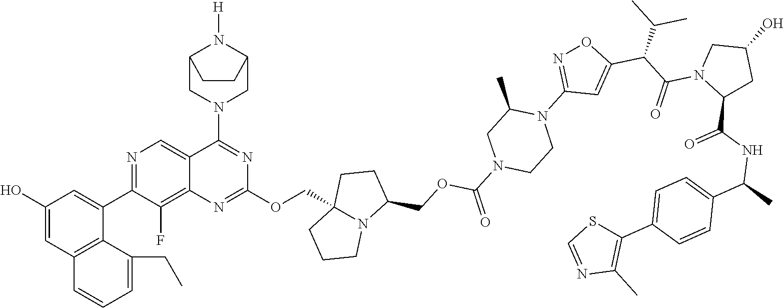 [SMILES: CCc1cccc2cc(O)cc(-c3ncc4c(N5CC6CCC(C5)N6)nc(OC[C@@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)[C@H](C)C5)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2S)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide (HCl salt). (formic acid salt, white solid). LC/MS (ESI) m/z: 1206.0 [M+H]⁺. ¹H NMR (400 MHz, CD₃OD) δ 9.11 (s, 1H), 8.90-8.84 (m, 1H), 8.46 (s, 1H), 7.62 (d, J=8.0 Hz, 1H), 7.50-7.31 (m, 5H), 7.31-7.27 (m, 1H), 7.16 (d, J=6.8 Hz, 1H), 7.03-6.98 (m, 1H), 6.16-6.05 (m, 1H), 5.14-4.97 (m, 1H), 4.83-4.77 (m, 1H), 4.75-4.67 (m, 2H), 4.63-4.52 (m, 3H), 4.46-4.32 (m, 2H), 4.28-4.16 (m, 1H), 4.12-4.02 (m, 1H), 4.01-3.93 (m, 2H), 3.92-3.60 (m, 7H), 3.56-3.45 (m, 1H), 3.43-3.35 (m, 1H), 3.19-2.89 (m, 3H), 2.50-2.44 (m, 3H), 2.44-1.85 (m, 18H), 1.61-1.42 (m, 3H), 1.15-0.93 (m, 6H), 0.93-0.80 (m, 6H).

Exemplary Synthesis of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (3R)-4-[5-[(1R)-1-[(2S,4R)-4-hydroxy-2-[[(1S)-1-[4-(4-methyl thiazol-5-yl)phenyl]ethyl]carbamoyl]pyrrolidine-1-carbonyl]-isobutyl]isoxazol-3-yl]-3-methyl-piperazine-1-carboxylate (Compound 327)

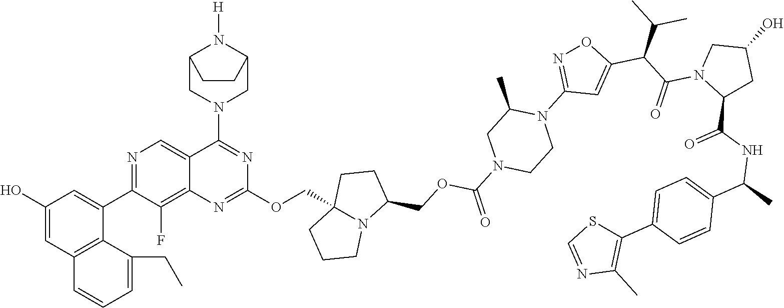 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)[C@H](C)C5)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide (HCl, salt). (formic acid salt, white solid). LC/MS (ESI) m/z: 1205.4 [M+H]⁺. ¹H NMR (400 MHz, CD₃OD) δ 9.11 (s, 1H), 8.89-8.85 (m, 1H), 8.50 (s, 1H), 7.63 (d, J=8.4 Hz, 1H), 7.47-7.32 (m, 5H), 7.31-7.27 (m, 1H), 7.17 (d, J=6.8 Hz, 1H), 7.01 (d, J=2.4 Hz, 1H), 6.13-6.03 (m, 1H), 5.07-4.99 (m, 1H), 4.77-4.71 (m, 1H), 4.70-4.62 (m, 2H), 4.59-4.47 (m, 3H), 4.46-4.36 (m, 2H), 4.19-4.04 (m, 2H), 4.00-3.72 (m, 7H), 3.68-3.50 (m, 2H), 3.47-3.34 (m, 2H), 3.22-3.08 (m, 2H), 3.06-2.96 (m, 1H), 2.50-2.44 (m, 3H), 2.43-1.81 (m, 18H), 1.60-1.49 (m, 3H), 1.11 (d, J=6.4 Hz, 3H), 1.05 (d, J=6.4 Hz, 3H), 0.94-0.84 (m, 6H).

Exemplary Synthesis of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]-2-methyl-piperazine-1-carboxylate (Compound 326)

Step 1: Preparation of (2S,4R)-4-hydroxy-1-[(2S)-3-methyl-2-[3-[(3R)-3-methylpiperazin-1-yl]isoxazol-5-yl]butanoyl]-N-[(1S)-1-[4-(4-methylthiazol-5-yl)phenyl]ethyl]pyrrolidine-2-carboxamide

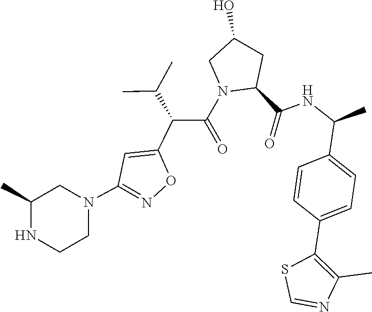

The title compound was prepared in an analogous manner to (2S,4R)-1-[(2S)-2-[3-[(2S,5R)-2,5-dimethylpiperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl (2S)-2-methylpiperazine-1-carboxylate. (HCl salt, light yellow solid). LC/MS (ESI) m/z: 581.3 [M+H]⁺.

Step 2: Preparation of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]-2-methyl-piperazine-1-carboxylate

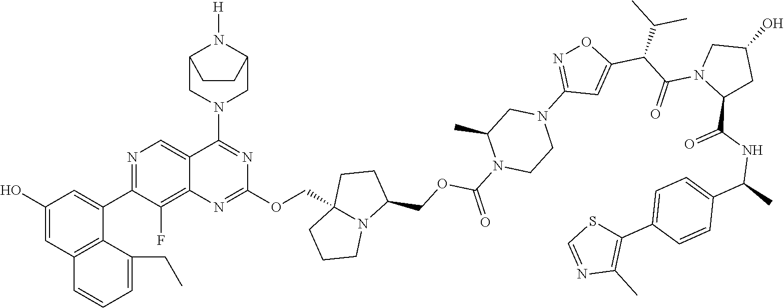 [SMILES: CCc1cccc2cc(O)cc(-c3ncc4c(N5CC6CCC(C5)N6)nc(OC[C@@]56CCCN5[C@H](COC(=O)N5CCN(c7cc([C@@H](C(=O)N8C[C@H](O)C[C@H]8C(=O)N[C@@H](C)c8ccc(-c9scnc9C)cc8)C(C)C)on7)C[C@@H]5C)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2S)-3-methyl-2-[3-[(3S)-3-methylpiperazin-1-yl]isoxazol-5-yl]butanoyl]-N-[(1S)-1-[4-(4-methylthiazol-5-yl)phenyl]ethyl]pyrrolidine-2-carboxamide (HCl salt). (formic acid salt, white solid). LC/MS (ESI) m/z: 1205.4 [M+H]⁺. ¹H NMR (400 MHz, CD₃OD) δ 9.09 (s, 1H), 8.88-8.87 (m, 1H), 7.63-7.61 (m, 1H), 7.37-7.29 (m, 6H), 7.16-7.14 (m, 1H), 7.00-6.99 (m, 1H), 6.14 (s, 1H), 4.98-4.96 (m, 1H), 4.73-4.67 (m, 3H), 4.59-4.55 (m, 2H), 4.43-4.22 (m, 4H), 3.94-3.79 (m, 5H), 3.73-3.34 (m, 7H), 3.25-3.17 (m, 1H), 2.98-2.90 (m, 1H), 2.77-2.72 (m, 1H), 2.45 (s, 3H), 2.39-2.18 (m, 8H), 2.13-1.89 (m, 10H), 1.48-1.45 (m, 3H), 1.26-1.19 (m, 3H), 1.06-1.04 (m, 3H), 0.91-0.85 (m, 6H).

Exemplary Synthesis of [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (2S)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]-2-methyl-piperazine-1-carboxylate (Compound 325)

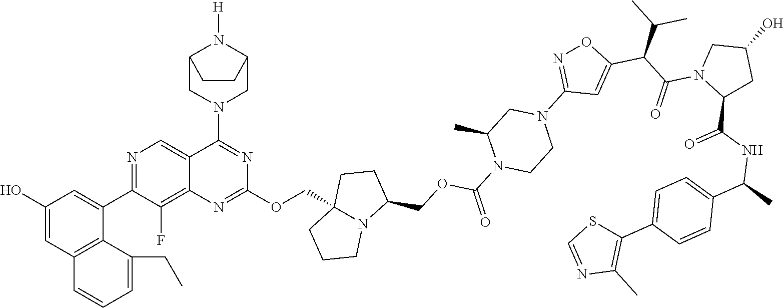 [SMILES: CCc1cccc2cc(O)cc(-c3ncc4c(N5CC6CCC(C5)N6)nc(OC[C@@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)C[C@@H]5C)CC6)nc4c3F)c12]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from tert-butyl 3-[7-[8-ethyl-3-(methoxymethoxy)-1-naphthyl]-8-fluoro-2-[[(3S,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-[3-[(3S)-3-methylpiperazin-1-yl]isoxazol-5-yl]butanoyl]-N-[(1S)-1-[4-(4-methylthiazol-5-yl)phenyl]ethyl]pyrrolidine-2-carboxamide (HCl salt). (formic acid salt, white solid). LC/MS (ESI) m/z: 1205.4 [M+H]⁺. ¹H NMR (400 MHz, CD₃OD) δ 9.11-9.10 (m, 1H), 8.88 (s, 1H), 7.64-7.62 (m, 1H), 7.45-7.29 (m, 6H), 7.17-7.15 (m, 1H), 7.02-7.00 (m, 1H), 6.14 (s, 1H), 5.04-5.02 (m, 1H), 4.70-4.63 (m, 3H), 4.57-4.40 (m, 6H), 4.14-4.13 (m, 1H), 4.00-3.97 (m, 1H), 3.86-3.74 (m, 5H), 3.65-3.60 (m, 3H), 3.48-3.44 (m, 2H), 3.25 (s, 1H), 3.05-3.02 (m, 1H), 2.87-2.81 (m, 1H), 2.47 (s, 3H), 2.41-2.25 (m, 5H), 2.23-1.87 (m, 13H), 1.52-1.50 (m, 3H), 1.26-1.24 (m, 3H), 1.05-1.04 (m, 3H), 0.91-0.86 (m, 6H).

Exemplary Synthesis of (2S,4R)-1-[(2S)-2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 339) and (2S,4R)-1-[(2R)-2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 338)

Step 1: Preparation of 4-(azepan-1-yl)-7-chloro-8-fluoro-2-methylsulfanyl-pyrido[4,3-d]pyrimidine

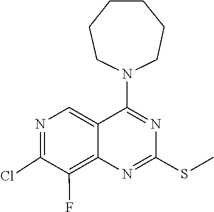 [SMILES: CSc1nc(N2CCCCCC2)c2cnc(Cl)c(F)c2n1]

To a solution of 4,7-dichloro-8-fluoro-2-methylsulfanyl-pyrido[4,3-d]pyrimidine (2.15 g, 8.14 mmol, 1.0 eq) and DIEA (40.70 mmol, 7.1 mL, 5.0 eq) in CH₂Cl₂ (30.0 mL) at −40° C. was added a solution of azepane (12.21 mmol, 1.4 mL, 1.5 eq) in CH₂Cl₂ (10.0 mL), and the reaction mixture was stirred at −40° C. for 30 minutes. The crude product was triturated with a mixture of petroleum ether (30 mL) and EtOAc (10.0 mL). The resulting mixture was filtered, and the filtrate was evaporated to dryness. Purification by flash chromatography on SiO₂ (gradient: 0˜17% EtOAc in petroleum ether) afforded 4-(azepan-1-yl)-7-chloro-8-fluoro-2-methylsulfanyl-pyrido[4,3-d]pyrimidine (1.9 g, 4.78 mmol, 59% yield) as a brown solid. LC/MS (ESI) m/z: 327.0 [M+H]⁺. ¹H NMR (400 MHz, CD₃OD) δ 8.97-8.90 (m, 1H), 4.01-3.93 (m, 4H), 2.54 (s, 3H), 1.89 (br s, 4H), 1.55 (br s, 4H).

Step 2: Preparation of 4-(azepan-1-yl)-7-[8-ethyl-3-(methoxymethoxy)-1-naphthyl]-8-fluoro-2-methylsulfanyl-pyrido[4,3-d]pyrimidine

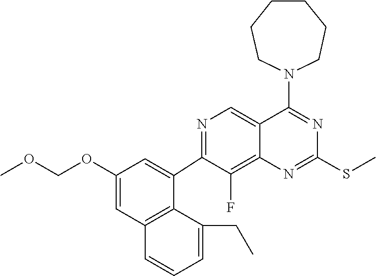

To a solution of 4-(azepan-1-yl)-7-chloro-8-fluoro-2-methylsulfanyl-pyrido[4,3-d]pyrimidine (1.6 g, 4.78 mmol, 1.0 eq) and 2-[8-ethyl-3-(methoxymethoxy)-1-naphthyl]-4,4,5,5-tetramethyl-1,3,2-dioxaborolane (2.0 g, 5.87 mmol, 1.2 eq) in dioxane (30.0 mL) were added K₃PO₄ (3 M, 4.9 mL, 3.0 eq) and [2-(2-aminophenyl)phenyl]palladium (1+); bis(1-adamantyl)-butyl-phosphane; methanesulfonate (356 mg, 0.489 mmol, 0.1 eq), and the reaction mixture was stirred at 80° C. for 16 hours. The mixture was extracted with ethyl acetate (3×20 mL), and the combined organic extracts were washed brine (20.0 mL), dried over Na₂SO₄, filtered, and concentrated under reduced pressure. The crude product was purified by flash chromatography on SiO₂ (gradient: 0˜22% EtOAc in petroleum ether) to afford 4-(azepan-1-yl)-7-[8-ethyl-3-(methoxymethoxy)-1-naphthyl]-8-fluoro-2-methylsulfanyl-pyrido[4,3-d]pyrimidine (1.99 g, 3.16 mmol, 64% yield) as a brown solid. LC/MS (ESI) m/z: 507.1 [M+H]⁺.

Step 3: Preparation of 4-(azepan-1-yl)-7-[8-ethyl-3-(methoxymethoxy)-1-naphthyl]-8-fluoro-2-methylsulfonyl-pyrido[4,3-d]pyrimidine

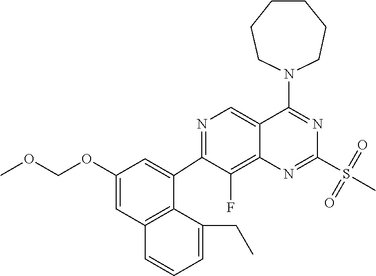

To a solution of 4-(azepan-1-yl)-7-[8-ethyl-3-(methoxymethoxy)-1-naphthyl]-8-fluoro-2-methylsulfanyl-pyrido[4,3-d]pyrimidine (1.99 g, 3.16 mmol, 1.0 eq) in DMF (20.0 mL) was added 4 Å MS (2.0 g, 3.93 mmol, 1.0 eq), and the resulting mixture was stirred at 30° C. for 16 hours. Oxone (7.2 g, 11.78 mmol, 3.0 eq) was then added, and the reaction mixture was stirred at 30° C. for 8 hours. The reaction was quenched by addition of saturated Na₂SO₃/NaHCO₃ solution (20.0 mL/20.0 mL). The mixture was filtered through celite pad, diluted with water (30.0 mL), and then extracted with EtOAc (3×30 mL). The combined organic extracts were washed brine (3×15.0 mL), dried over Na₂SO₄, filtered, and concentrated. The crude product was triturated with a mixture of EtOAc (20 mL) and petroleum ether (60.0 mL) at 20° C. for 10 minutes. The mixture was filtered, the solid was evaporated to dryness, and the filtrate was evaporated to dryness for further purification. The crude product was purified by flash chromatography on SiO₂ (gradient 0˜28% EtOAc in petroleum ether) to afford 4-(azepan-1-yl)-7-[8-ethyl-3-(methoxymethoxy)-1-naphthyl]-8-fluoro-2-methylsulfonyl-pyrido[4,3-d]pyrimidine (2 g, 2.82 mmol, 60% yield) as a yellow solid. LC/MS (ESI) m/z: 539.2 [M+H]⁺.

Step 4: Preparation of methyl 2-[3-[[(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-isovalerate

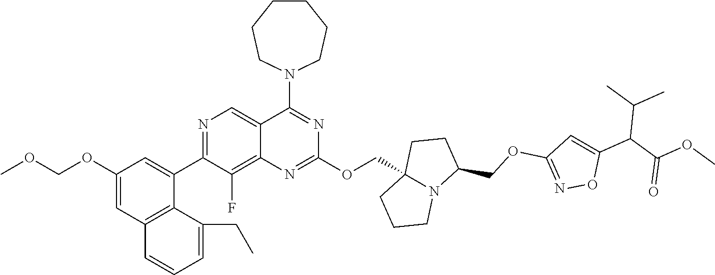

To a solution of 4-(azepan-1-yl)-7-[8-ethyl-3-(methoxymethoxy)-1-naphthyl]-8-fluoro-2-methylsulfonyl-pyrido[4,3-d]pyrimidine (734 mg, 1.36 mmol, 1.2 eq) and methyl 2-[3-[[(3S,8S)-8-(hydroxymethyl)-1,2,3,5,6,7-hexahydropyrrolizin-3-yl]methoxy]isoxazol-5-yl]-isovalerate (400 mg FA salt, 1.13 mmol, 1.0 eq) in toluene (10 mL) were added DIEA (147 mg, 1.13 mmol, 1.0 eq) and 4 Å molecular sieves (1.0 g), and the resulting mixture was stirred at 20° C. for 30 minutes. t-BuONa (218 mg, 2.27 mmol, 2.0 eq) was then added at 0° C., and the reaction mixture was stirred at 0° C. for 30 more minutes. Water/2N aqueous HCl (20 mL/0.85 mL) was added, and the resulting mixture extracted with EtOAc (30 mL×3). The combined extracts were washed with brine (50 mL), dried over anhydrous Na₂SO₄, filtered, and concentrated under reduced pressure. The resulting residue was purified by flash chromatography on SiO₂ (gradient: 0˜3% methanol in dichloromethane) to afford methyl 2-[3-[[(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-isovalerate (601 mg, 0.741 mmol, 65% yield) as a yellow solid. LC/MS (ESI) m/z: 811.3 [M+H]⁺.

Step 5: Preparation of 2-[3-[[(3S,8S)-8-[[4-(azepan-1-yl)-7-[8-eth yl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-isovaleric acid

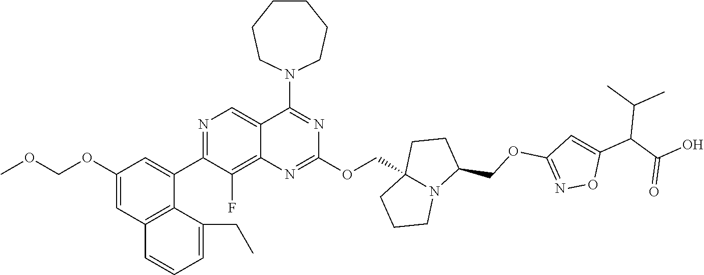 [SMILES: CCc1cccc2cc(OCOC)cc(-c3ncc4c(N5CCCCCC5)nc(OC[C@@]56CCCN5[C@H](COc5cc(C(C(=O)O)C(C)C)on5)CC6)nc4c3F)c12]

To a solution of methyl 2-[3-[[(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-isovalerate (601 mg, 0.741 mmol, 1.0 eq) in THF (3.0 mL) was added a solution of LiOH·H₂O (93.3 mg, 2.22 mmol, 3.0 eq) in water (3.0 mL), and the reaction mixture was stirred at 20° C. for 16 hours. The mixture was diluted with water (15 mL), and the pH adjusted to pH=5 using 2N hydrochloric acid. The resulting mixture was extracted with EtOAc (15 mL×3), and the combined extracts were washed with brine (20 mL), dried over anhydrous Na₂SO₄, filtered, and concentrated under reduced pressure to give 2-[3-[[(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-isovaleric acid (585 mg, 0.734 mmol, 99% yield) as yellow solid. LC/MS (ESI) m/z: 797.4 [M+H]⁺.

Step 6: Preparation of (2S,4R)-1-[2-[3-[[(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

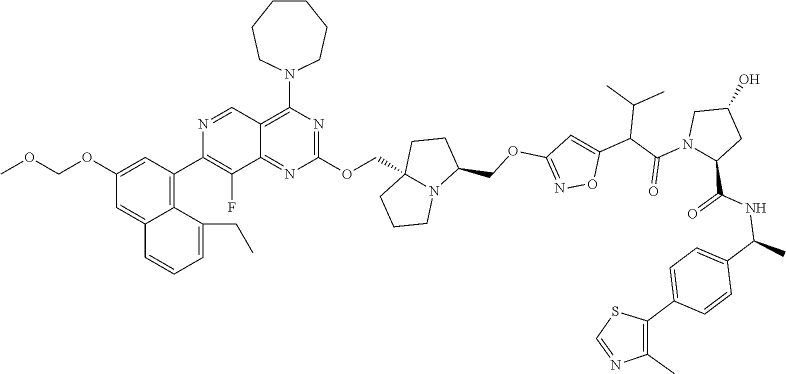 [SMILES: CCc1cccc2cc(OCOC)cc(-c3ncc4c(N5CCCCCC5)nc(OC[C@@]56CCCN5[C@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

To a solution of 2-[3-[[(3S,8S)-8-[[4-(azepan-1-yl)-7-[8-ethyl-3-(methoxy methoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-isovaleric acid (585 mg, 0.734 mmol, 1.0 eq) in CH₂Cl₂ (20 mL) were added DIEA (949 mg, 7.34 mmol, 10 eq), HATU (335 mg, 0.881 mmol, 1.2 eq), and (2S,4R)-4-hydroxy-N-[(1S)-1-[4-(4-methylthiazol-5-yl)phenyl]ethyl]pyrrolidine-2-carboxamide (540 mg HCl salt, 0.881 mmol, 1.2 eq), and the reaction mixture was stirred at 20° C. for 1 hour. The mixture was diluted water (20 mL) and the layers were separated. The aqueous layer was extracted with CH₂Cl₂ (20 mL), and the combined organic extracts were washed with brine (20 mL), dried over anhydrous Na₂SO₄, filtered, and concentrated under reduced pressure. The crude product was purified by flash chromatography on SiO₂ (gradient: 0˜4% CH₃OH (10% 7 M NH₃/CH₃OH) in CH₂Cl₂) to afford (2S,4R)-1-[2-[3-[[(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (641 mg, 0.577 mmol, 79% yield) as a yellow solid.

To adjust the diastereomer ratio, to a portion of this material (560 mg, 0.505 mmol) in dioxane (10 mL) was added Cs₂CO₃ (164 mg, 0.505 mmol), and the suspension was stirred at 70° C. for 16 hours. The mixture was filtered, and the filtrate was concentrated under reduced pressure to give 641 mg of diastereomeric mixture. LC/MS (ESI) m/z: 1110.9 [M+H]⁺.

Step 7: Preparation of (2S,4R)-1-[2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

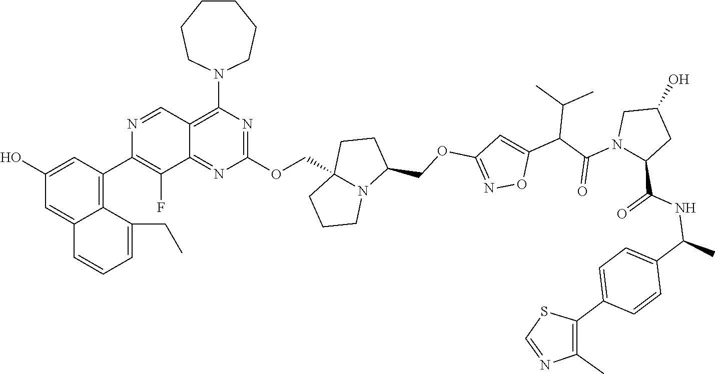 [SMILES: CCc1cccc2cc(O)cc(-c3ncc4c(N5CCCCCC5)nc(OC[C@@]56CCCN5[C@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

To a solution of (2S,4R)-1-[2-[3-[[(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (320 mg, 0.288 mmol, 1.0 eq) in CH₂Cl₂ (2.0 mL) was added 4 M HCl/dioxane (2.0 mL), and the reaction mixture was stirred at 20° C. for 30 minutes. The reaction mixture was concentrated under reduced pressure, and the resulting residue was dissolved in THF (30 mL). Triethylamine (0.20 mL) was added, and the resulting suspension was stirred at 20° C. for 30 minutes. The mixture was filtered, and the filtrate was concentrated under reduced pressure to give (2S,4R)-1-[2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (303 mg, 0.284 mmol, 99% yield) as a yellow solid. LC/MS (ESI) m/z: 1066.5 [M+H]⁺.

Step 8: Preparation of (2S,4R)-1-[(2S)-2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide and (2S,4R)-1-[(2R)-2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

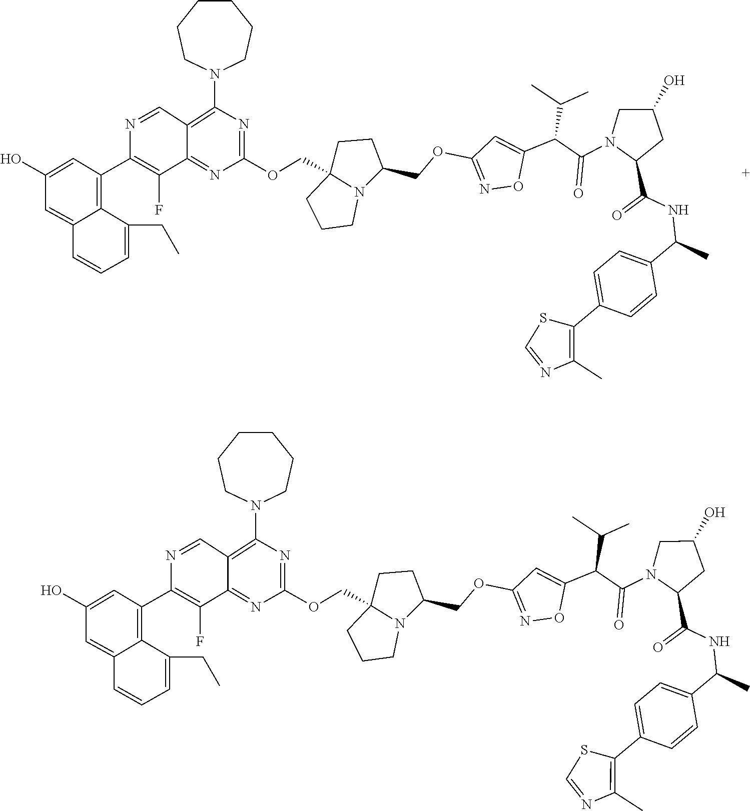 [SMILES: CCc1cccc2cc(O)cc(-c3ncc4c(N5CCCCCC5)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12 ; CCc1cccc2cc(O)cc(-c3ncc4c(N5CCCCCC5)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

The diastereomeric mixture was separated by chiral SFC {Column: Daicel Chiralpak IA (250 mm*30 mm, 10 um); Mobile phase: [30% CH₃CN in EtOH (0.1% NH₄OH)]}. Fraction 1 was concentrated under reduced pressure at 35° C. to give (2S,4R)-1-[(2S)-2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (113.6 mg, 97.31% purity, 0.104 mmol) as a white solid. LC/MS (ESI) m/z: 1066.3 [M+H]⁺. ¹H NMR (400 MHz, CD₃OD) δ 9.13 (s, 1H), 8.91-8.82 (m, 1H), 7.62 (d, J=8.0 Hz, 1H), 7.50-7.32 (m, 5H), 7.28 (d, J=2.4 Hz, 1H), 7.15 (d, J=6.8 Hz, 1H), 7.04-7.01 (m, 1H), 6.06-5.99 (m, 1H), 5.13-4.93 (m, 1H), 4.59-4.54 (m, 1H), 4.51-4.32 (m, 5H), 4.17-4.06 (m, 4H), 3.97-3.83 (m, 1H), 3.79-3.54 (m, 3H), 3.17-2.91 (m, 2H), 2.52-2.44 (m, 3H), 2.42-1.85 (m, 17H), 1.74-1.66 (m, 4H), 1.59-1.43 (m, 3H), 1.10-0.95 (m, 3H), 0.92-0.82 (m, 6H).

Fraction 2 was concentrated under reduced pressure at 35° C. to give (2S,4R)-1-[(2R)-2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (95.8 mg, 98.55% purity, 0.089 mol) as a white solid. LC/MS (ESI) m/z: 1066.3 [M+H]⁺. ¹H NMR (400 MHz, CD₃OD) δ 9.15-9.10 (m, 1H), 8.88-8.85 (m, 1H), 7.62 (d, J=8.4 Hz, 1H), 7.47-7.32 (m, 5H), 7.28 (d, J=2.8 Hz, 1H), 7.16 (d, J=6.8 Hz, 1H), 7.03 (d, J=2.8 Hz, 1H), 6.04-5.96 (m, 1H), 5.03 (q, J=7.2 Hz, 1H), 4.54-4.35 (m, 5H), 4.34-4.26 (m, 1H), 4.16-4.05 (m, 4H), 3.87-3.78 (m, 1H), 3.73-3.46 (m, 3H), 3.01-2.80 (m, 2H), 2.49-2.43 (m, 3H), 2.42-2.16 (m, 5H), 2.11-2.00 (m, 5H), 1.99-1.81 (m, 6H), 1.76-1.67 (m, 5H), 1.61-1.49 (m, 3H), 1.05 (d, J=6.4 Hz, 3H), 0.94-0.85 (m, 6H).

Exemplary Synthesis of &, (2S,4R)-1-[(2S)-2-[3-[[(3S,8S)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 315) & (2S,4R)-1-[(2R)-2-[3-[[(3S,8S)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 314)

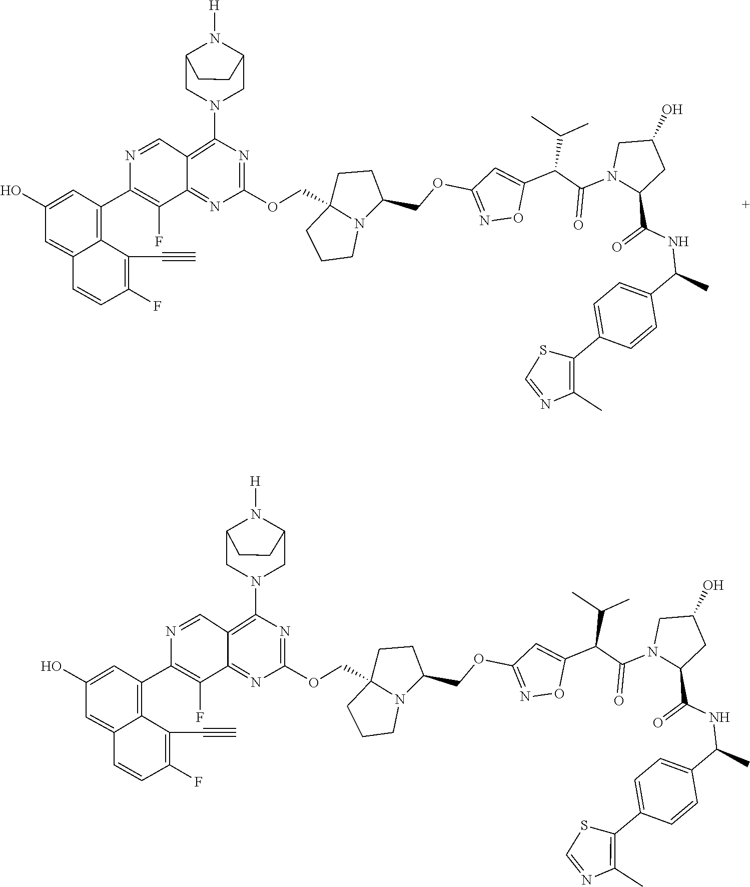 [SMILES: C#Cc1c(F)ccc2cc(O)cc(-c3ncc4c(N5CC6CCC(C5)N6)nc(OC[C@@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12 ; C#Cc1c(F)ccc2cc(O)cc(-c3ncc4c(N5CC6CCC(C5)N6)nc(OC[C@@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compounds were made in an analogous manner to (2S,4R)-1-[(2S)-2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide and (2S,4R)-1-[(2R)-2-[3-[[(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate and methyl 2-[3-[[(3S,8S)-8-(hydroxymethyl)-1,2,3,5,6,7-hexahydropyrrolizin-3-yl]methoxy]isoxazol-5-yl]-isovalerate.

(2S,4R)-1-[(2S)-2-[3-[[(3S,8S)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methyl thiazol-5-yl)phenyl]ethyl]pyrrolidine-2-carboxamide was obtained as a yellow solid. LC/MS (ESI) m/z: 1093.3 [M+H]⁺. ¹H NMR (400 MHz, CD₃OD) δ 9.01 (s, 1H), 8.86 (s, 1H), 7.87-7.83 (m, 1H), 7.43-7.30 (m, 6H), 7.21 (d, J=2.4 Hz, 1H), 6.05 (s, 1H), 4.97 (q, J=7.2 Hz, 1H), 4.59-4.25 (m, 8H), 3.76-3.64 (m, 8H), 3.36 (d, J=2.0 Hz, 1H), 2.97-2.82 (m, 2H), 2.50-2.45 (m, 3H), 2.26-2.13 (m, 3H), 1.99-1.77 (m, 12H), 1.48 (d, J=7.2 Hz, 3H), 1.05 (d, J=6.4 Hz, 3H), 0.91 (d, J=6.4 Hz, 3H).

(2S,4R)-1-[(2R)-2-[3-[[(3S,8S)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxy methyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide was obtained as a yellow solid. LC/MS (ESI) m/z: 1093.9 [M+H]⁺. ¹H NMR (400 MHz, CD₃OD) δ 9.01 (s, 1H), 8.88 (s, 1H), 7.92-7.80 (m, 1H), 7.47-7.36 (m, 4H), 7.36-7.27 (m, 2H), 7.21 (d, J=2.4 Hz, 1H), 6.03 (d, J=4.4 Hz, 1H), 5.03 (q, J=6.8 Hz, 1H), 4.69-4.56 (m, 2H), 4.53-4.34 (m, 5H), 4.31-4.25 (m, 1H), 3.88-3.56 (m, 8H), 3.36 (d, J=2.8 Hz, 1H), 3.01-2.76 (m, 2H), 2.51-2.44 (m, 3H), 2.28-2.07 (m, 3H), 2.04-1.67 (m, 12H), 1.52 (d, J=7.2 Hz, 3H), 1.05 (d, J=6.4 Hz, 3H), 0.91-0.85 (m, 3H).

Exemplary Synthesis of (2S,4R)-1-[(2R)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 324) and (2S,4R)-1-[(2S)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 323)

Step 1: Preparation of [(3S,8R)-3-[[tert-butyl(diphenyl)silyl]oxymethyl]-1,2,3,5,6,7-hexahydropyrrolizin-8-yl]methyl benzoate

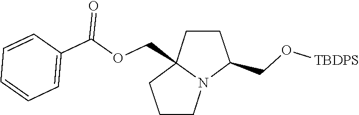

To a mixture of [(3S,8R)-3-[[tert-butyl(diphenyl)silyl]oxymethyl]-1,2,3,5,6,7-hexahydropyrrolizin-8-yl]methanol (5.80 g, 14.16 mmol, 1.0 eq), triethylamine (5.9 mL, d=0.727 g/mL, 42.48 mmol, 3.0 eq), and DMAP (173 mg, 1.42 mmol, 0.10 eq) in CH₂Cl₂ (50 mL) 0° C. under N₂ was added a solution of BzCl (1.8 mL, d=0.727 g/mL, 15.57 mmol, 1.1 eq) in CH₂Cl₂ (5.0 mL), and the reaction mixture was stirred at 20° C. for 12 hours. The mixture was diluted with water (40 mL), and the organic layer was separated. The aqueous layer was extracted with dichloromethane (3×40 mL), and the combined organic extracts were washed with brine (30 mL), dried over anhydrous Na₂SO₄, filtered, and concentrated under reduced pressure. The resulting residue was purified by flash chromatography on SiO₂ (gradient: 0˜10% EtOAc in petroleum ether) to afford [(3S,8R)-3-[[tert-butyl(diphenyl)silyl]oxymethyl]-1,2,3,5,6,7-hexahydropyrrolizin-8-yl]methyl benzoate (6.50 g, 11.89 mmol, 84% yield) as a colorless gum.

Step 2: Preparation of [(3S,8R)-3-(hydroxymethyl)-1,2,3,5,6,7-hexahydropyrrolizin-8-yl]methyl benzoate

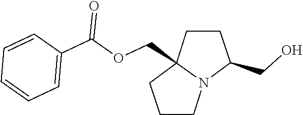 [SMILES: O=C(OC[C@]12CCCN1[C@H](CO)CC2)c1ccccc1]

To a mixture of [(3S,8R)-3-[[tert-butyl(diphenyl)silyl]oxymethyl]-1,2,3,5,6,7-hexahydropyrrolizin-8-yl]methyl benzoate (6.50 g, 94% purity, 11.89 mmol, 1.0 eq) in dichloromethane (60 mL) at 20° C. was added trimethylamine trihydrofluoride (30.6 g, 189.78 mmol, 15.96 eq), and the reaction mixture was stirred at 20° C. for 2 hours. The mixture was concentrated under reduced pressure, and the resulting residue was diluted in water (50 mL) and washed with methyl tert-butyl ether (3×40 mL). The aqueous phase was basified with saturated aqueous NaHCO₃ solution until pH=8 and further extracted with CH₂Cl₂ (5×40 mL). The combined organic extracts were washed with brine (40 mL), dried over anhydrous Na₂SO₄, filtered, and concentrated to give [(3S,8R)-3-(hydroxymethyl)-1,2,3,5,6,7-hexahydropyrrolizin-8-yl]methyl benzoate (3.40 g, 9.14 mmol, 77% yield) as a colorless gum. LC/MS (ESI) m/z: 276.0 [M+H]⁺.

Step 3: Preparation of [(3S,8R)-3-[[5-(1-methoxycarbonyl-isobutyl)isoxazol-3-yl]oxymethyl]-1,2,3,5,6,7-hexahydropyrrolizin-8-yl]methyl benzoate and [(6R,8aR)-6-[5-(1-methoxycarbonyl-isobutyl)isoxazol-3-yl]oxy-2,3,5,6,7,8-hexahydro-1H-indolizin-8a-yl]methyl benzoate

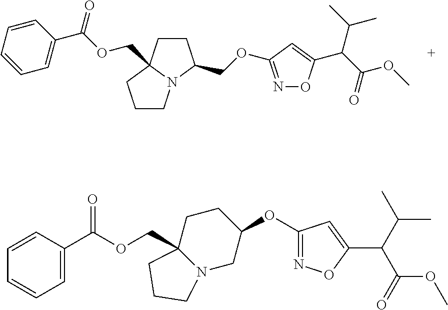 [SMILES: COC(=O)C(c1cc(OC[C@@H]2CC[C@@]3(COC(=O)c4ccccc4)CCCN23)no1)C(C)C ; COC(=O)C(c1cc(O[C@@H]2CC[C@@]3(COC(=O)c4ccccc4)CCCN3C2)no1)C(C)C]

To a mixture of [(3S,8R)-3-(hydroxymethyl)-1,2,3,5,6,7-hexahydropyrrolizin-8-yl]methyl benzoate (2.90 g, 74% purity, 7.79 mmol, 1.0 eq), methyl 2-(3-hydroxy isoxazol-5-yl)-isovalerate (2.10 g, 10.53 mmol, 1.35 eq) and PPh₃ (5.50 g, 21.06 mmol, 2.7 eq) in THF (40 mL) was added DIAD (4.1 mL, d=1.04 g/mL, 21.06 mmol, 2.7 eq) at −10° C. under N₂, and the reaction mixture was stirred at −10° C. for 3 hours. The mixture was diluted with water (30 mL) and extracted with ethyl acetate (3×40 mL). The combined extracts were washed with brine (20 mL), dried with anhydrous Na₂SO₄, filtered, and concentrated. The resulting residue was purified by flash chromatography on SiO₂ (gradient: 0˜15% ethyl acetate in petroleum ether) to afford the products (4.40 g) as a yellow gum. The mixture was separated by chiral SFC [Column: Daicel Chiralpak AD (250 mm*50 mm, 10 um); Mobile phase: [30% isopropanol]. Fraction 1 was concentrated under reduced pressure to afford [(6R,8aR)-6-[5-(1-methoxycarbonyl-isobutyl)isoxazol-3-yl]oxy-2,3,5,6,7,8-hexahydro-1H-indolizin-8a-yl]methyl benzoate (822 mg, 1.62 mmol) as yellow gum. LC/MS (ESI) m/z: 457.2 [M+H]⁺. ¹H NMR (400 MHz, CDCl₃) δ 8.10-8.01 (m, 2H), 7.63-7.54 (m, 1H), 7.50-7.42 (m, 2H), 5.87 (s, 1H), 4.89 (td, J=4.8, 9.6 Hz, 1H), 4.60 (d, J=6.0 Hz, 1H), 4.32-4.17 (m, 1H), 3.73 (s, 3H), 3.49 (d, J=8.8 Hz, 1H), 3.35 (d, J=10.0 Hz, 1H), 3.20-3.03 (m, 2H), 2.95 (d, J=2.8 Hz, 1H), 2.40-2.30 (m, 1H), 2.22 (dd, J=9.2, 3.6 Hz, 1H), 1.99-1.85 (m, 3H), 1.85-1.54 (m, 4H), 1.00 (d, J=6.4 Hz, 3H), 0.93 (d, J=6.8 Hz, 3H).

Fraction 2 was concentrated under reduced pressure to afford [(3S,8R)-3-[[5-(1-methoxycarbonyl-isobutyl)isoxazol-3-yl]oxymethyl]-1,2,3,5,6,7-hexahydropyrrolizin-8-yl]methyl benzoate (3.10 g 6.23 mmol) as a yellow gum. LC/MS (ESI) m/z: 457.2 [M+H]⁺. ¹H NMR (400 MHz, CDCl₃) δ 8.07 (d, J=7.6 Hz, 2H), 7.62-7.52 (m, 1H), 7.49-7.42 (m, 2H), 5.90 (s, 1H), 4.36-4.12 (m, 4H), 3.73 (s, 3H), 3.49 (d, J=8.8 Hz, 1H), 3.23-3.13 (m, 2H), 2.96-2.81 (m, 1H), 2.42-2.28 (m, 1H), 2.24-2.13 (m, 1H), 1.97-1.82 (m, 4H), 1.77-1.58 (m, 3H), 1.00 (d, J=6.8 Hz, 3H), 0.92 (d, J=6.8 Hz, 3H).

Step 4: Preparation of 2-[3-[[(3S,8R)-8-(hydroxymethyl)-1,2,3,5,6,7-hexahydropyrrolizin-3-yl]methoxy]isoxazol-5-yl]-isovaleric acid

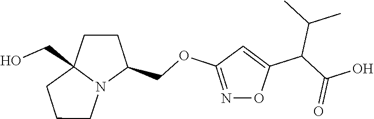 [SMILES: CC(C)C(C(=O)O)c1cc(OC[C@@H]2CC[C@@]3(CO)CCCN23)no1]

To [(3S,8R)-3-[[5-(1-methoxycarbonyl-isobutyl)isoxazol-3-yl]oxy methyl]-1,2,3,5,6,7-hexahydropyrrolizin-8-yl]methyl benzoate (3.10 g, 92% purity, 6.23 mmol, 1.0 eq) in THF (36 mL) and H₂O (12 mL) was added LiOH·H₂O (1.4 g, 33.84 mmol, 5.43 eq) in one portion at 25° C., and the reaction mixture was stirred at 25° C. for 12 hours. The mixture was concentrated under reduced pressure to remove THE, and then washed with methyl tert-butyl ether (3×30 mL). The resulting aqueous phase was adjusted to pH ˜3 with 2 M hydrochloric acid, then lyophilized to afford 2-[3-[[(3S,8R)-8-(hydroxymethyl)-1,2,3,5,6,7-hexahydropyrrolizin-3-yl]meth oxy]isoxazol-5-yl]-isovaleric acid (2.2 g, crude, HCl salt) as a yellow solid. LC/MS (ESI) m/z: 339.2 [M+H]⁺.

Step 5: Preparation of (2S,4R)-4-[tert-butyl(diphenyl)silyl]oxy-1-[2-[3-[[(3S,8R)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide

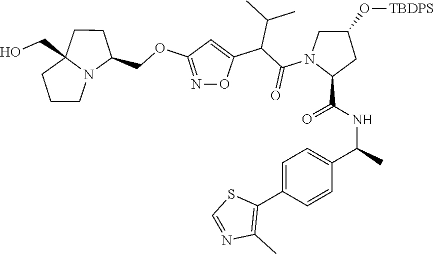

To a solution of 2-[3-[[(3S,8R)-8-(hydroxymethyl)-1,2,3,5,6,7-hexahydropyrrolizin-3-yl]meth oxy]isoxazol-5-yl]-isovaleric acid (2.2 g crude HCl salt, theoretically 5.85 mmol, 1.0 eq) and (2S,4R)-4-[tert-butyl(diphenyl)silyl]oxy-N-[(1S)-1-[4-(4-methyl thiazol-5-yl)phenyl]ethyl]pyrrolidine-2-carboxamide (4.3 g HCl salt, 7.04 mmol, 1.2 eq) in DMF (30 mL) were added DIEA (10.2 mL, d=0.742 g/mL, 58.70 mmol, 10.0 eq) and HATU (2.5 g, 6.46 mmol, 1.1 eq), and the resulting mixture was stirred at 25° C. for 2 hours. (2S,4R)-4-[tert-Butyl(diphenyl)silyl]oxy-N-[(1S)-1-[4-(4-methylthiazol-5-yl)phenyl]ethyl]pyrrolidine-2-carboxamide (1.0 g, 1.76 mmol, 0.30 eq) and HATU (669 mg, 1.76 mmol, 0.30 eq) were then added, and the reaction mixture was stirred at 25° C. for 1 more hour. The mixture was diluted with water (30 mL) and extracted with ethyl acetate (4×40 mL). The combined organic extracts were washed with water (6×10 mL) and brine (20 mL), dried over anhydrous Na₂SO₄, filtered, and concentrated. The residue was purified by flash chromatography on SiO₂ (gradient: 0˜3% methanol in CH₂Cl₂) to afford (2S,4R)-4-[tert-butyl(diphenyl)silyl]oxy-1-[2-[3-[[(3S,8R)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide (2.50 g, 2.70 mmol, 46% yield) as a yellow solid. LC/MS (ESI) m/z: 446.0 [M/2+H]⁺.

Step 6: Preparation of tert-butyl 3-[2-[[(3S,8R)-3-[[5-[1-[(2S,4R)-4-[tert-butyl(diphenyl)silyl]oxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

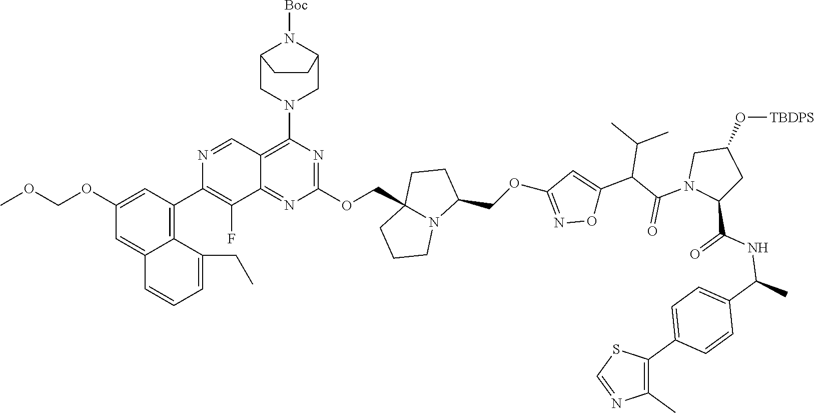

To a mixture of (2S,4R)-4-[tert-butyl(diphenyl)silyl]oxy-1-[2-[3-[[(3S,8R)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide (600 mg, 96% purity, 0.648 mmol, 1.0 eq), 4 Å molecular sieves (1.0 g), and tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (439 mg, 0.674 mmol, 1.04 eq) in THF (14 mL) was added t-BuONa (162 mg, 1.68 mmol, 2.59 eq) at 0° C. under N₂, and the reaction mixture was stirred at 0° C. for 1.5 hours. The mixture was treated with 2 M hydrochloric acid until pH=8, dried over anhydrous Na₂SO₄, filtered, and concentrated. The resulting residue was purified by flash chromatography on SiO₂ (gradient: 0˜2% methanol in dichloromethane) to afford tert-butyl 3-[2-[[(3S,8R)-3-[[5-[1-[(2S,4R)-4-[tert-butyl(diphenyl)silyl]oxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (847 mg, 0.469 mmol, 72% yield) as a yellow solid. LC/MS (ESI) m/z: 1462.6 [M+H]⁺.

Step 7: Preparation of tert-butyl 3-[7-[8-ethyl-3-(methoxy methoxy)-1-naphthyl]-8-fluoro-2-[[(3S,8R)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

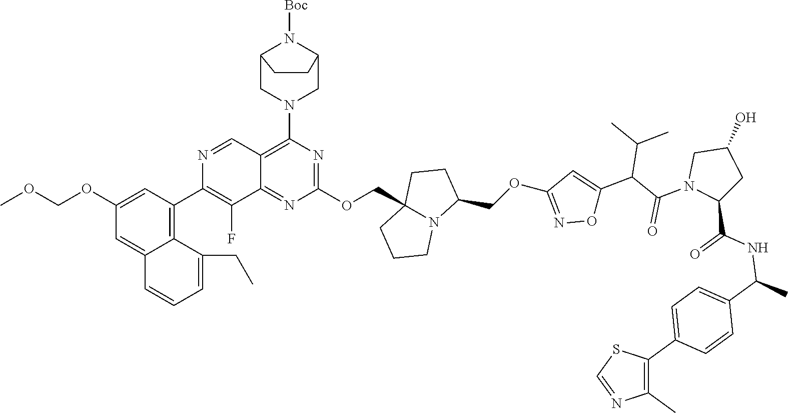 [SMILES: CCc1cccc2cc(OCOC)cc(-c3ncc4c(N5CC6CCC(C5)N6C(=O)OC(C)(C)C)nc(OC[C@]56CCCN5[C@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

To a solution of tert-butyl 3-[2-[[(3S,8R)-3-[[5-[1-[(2S,4R)-4-[tert-butyl(diphenyl) silyl]oxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-1,2,3,5,6,7-hexahydropyrrolizin-8-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (840 mg, 81% purity, 0.465 mmol, 1.0 eq) in DMF (12 mL) at 30° C. was added CsF (698 mg, 4.60 mmol, 8.0 eq), and the reaction mixture was stirred at 30° C. for 12 hours. The reaction mixture was diluted with water (20 mL) and extracted with ethyl acetate (3×30 mL). The combined organic extracts were washed with brine (5×15 mL), dried over anhydrous Na₂SO₄, filtered, and concentrated. The resulting residue was purified by flash chromatography on SiO₂ (gradient: 0˜7% methanol in dichloromethane) to afford tert-butyl 3-[7-[8-ethyl-3-(methoxymethoxy)-1-naphthyl]-8-fluoro-2-[[(3S,8R)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (596 mg, 0.458 mmol, 98% yield) as a yellow solid. LC/MS (ESI) m/z: 1223.7 [M+H]⁺.

Step 8: Preparation of (2S,4R)-1-[2-[3-[[(3S,8R)-8-[[4-(3,8-diaza bicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

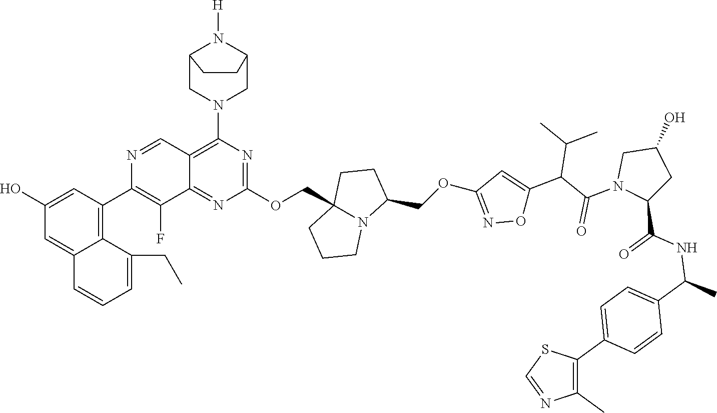 [SMILES: CCc1cccc2cc(O)cc(-c3ncc4c(N5CC6CCC(C5)N6)nc(OC[C@]56CCCN5[C@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

To tert-butyl 3-[7-[8-ethyl-3-(methoxymethoxy)-1-naphthyl]-8-fluoro-2-[[(3S,8R)-3-[[5-[1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (586 mg, 94% purity, 0.450 mmol) in CH₂Cl₂ (2.5 mL) at 25° C. was added 4 M HCl/dioxane (2.0 mL) in one portion, and the reaction mixture was stirred at 25° C. for 30 minutes. The mixture was concentrated, and the resulting residue was treated with trimethylamine until pH=9. THF was added, the resulting mixture filtered, and the filtrate was concentrated to give (2S,4R)-1-[2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (506 mg, 0.408 mmol, 91% yield) as a yellow solid. LC/MS (ESI) m/z: 1079.6 [M+H]⁺.

Step 9: Preparation of (2S,4R)-1-[(2R)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide and (2S,4R)-1-[(2S)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

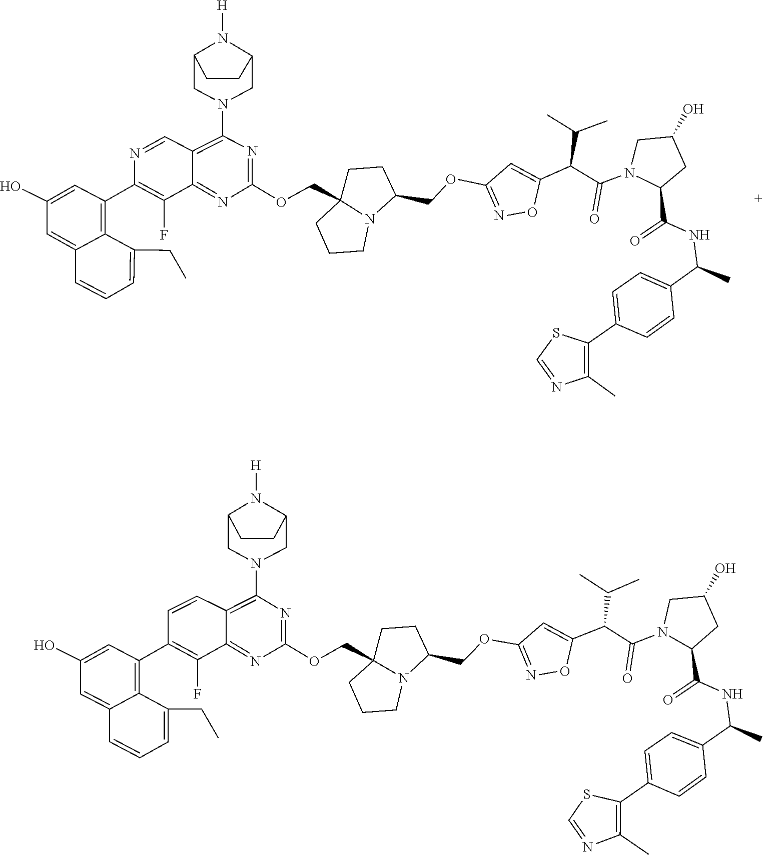 [SMILES: CCc1cccc2cc(O)cc(-c3ncc4c(N5CC6CCC(C5)N6)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12 ; CCc1cccc2cc(O)cc(-c3ccc4c(N5CC6CCC(C5)N6)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12]

(2S,4R)-1-[2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (470 mg, 0.379 mmol) was purified by chiral SFC {Column: Daicel Chiralpak IC (250 mm*30 mm, 10 um); Mobile phase: [35% CH₃CN in EtOH (0.1% NH₄OH)]}. Fraction 1 was concentrated under reduced pressure, washed with n-hexane (3×4 mL), and lyophilized to afford (2S,4R)-1-[(2R)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (161.4 mg, 0.145 mmol) as a white solid. LC/MS (ESI) m/z: 1080.0 [M+H]⁺. ¹H NMR (400 MHz, CD₃OD) δ 9.04 (d, J=8.8 Hz, 1H), 8.89-8.85 (m, 1H), 7.62 (d, J=8.3 Hz, 1H), 7.46-7.32 (m, 5H), 7.30-7.25 (m, 1H), 7.16 (d, J=7.2 Hz, 1H), 7.06-7.00 (m, 1H), 6.01-5.93 (m, 1H), 5.07-4.98 (m, 1H), 4.69-4.55 (m, 2H), 4.54-4.46 (m, 1H), 4.40 (s, 1H), 4.33-4.22 (m, 2H), 4.18-4.07 (m, 2H), 3.81-3.56 (m, 7H), 3.22-3.12 (m, 1H), 3.09-2.98 (m, 1H), 2.90-2.78 (m, 1H), 2.50-2.44 (m, 3H), 2.41-2.27 (m, 3H), 2.25-2.13 (m, 2H), 2.11-2.03 (m, 1H), 1.96-1.70 (m, 11H), 1.58-1.47 (m, 3H), 1.07-1.01 (m, 3H), 0.92-0.85 (m, 6H).

Fraction 2 was concentrated under reduced pressure, washed with n-hexane (3×4 mL), and lyophilized to afford (2S,4R)-1-[(2S)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (111.6 mg, 0.100 mmol) as a white solid. LC/MS (ESI) m/z: 1079.9 [M+H]⁺. ¹H NMR (400 MHz, CD₃OD) δ 9.03-8.99 (m, 1H), 8.90-8.84 (m, 1H), 7.62 (d, J=8.0 Hz, 1H), 7.44-7.38 (m, 2H), 7.37-7.31 (m, 3H), 7.28 (d, J=2.4 Hz, 1H), 7.14 (d, J=7.2 Hz, 1H), 7.03 (dd, J=10.8, 2.4, 1H), 6.04 (d, J=6.4 Hz, 1H), 5.01-4.94 (m, 1H), 4.66-4.59 (m, 2H), 4.59-4.55 (m, 1H), 4.45-4.38 (m, 1H), 4.31-4.20 (m, 2H), 4.16-4.06 (m, 2H), 3.78-3.73 (m, 1H), 3.72-3.61 (m, 6H), 3.20-3.10 (m, 1H), 3.02 (dd, J=10.0, 4.0 Hz, 1H), 2.89-2.76 (m, 1H), 2.49-2.45 (m, 3H), 2.40-2.26 (m, 3H), 2.24-2.18 (m, 2H), 2.04 (dd, J=12.4, 6.8 Hz, 1H), 1.96-1.74 (m, 11H), 1.47 (d, J=7.2 Hz, 3H), 1.04 (d, J=6.4 Hz, 3H), 0.92-0.86 (m, 6H).

Exemplary Synthesis of (2S,4R)-1-[(2R)-2-[3-[[(3R,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 324)

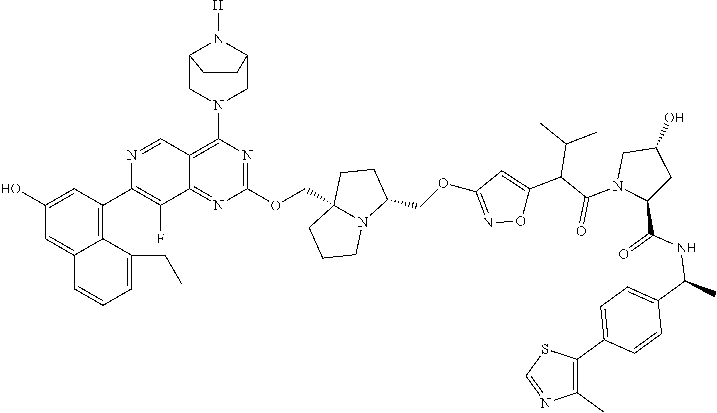 [SMILES: CCc1cccc2cc(O)cc(-c3ncc4c(N5CC6CCC(C5)N6)nc(OC[C@@]56CCCN5[C@@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

The title compound was made in an analogous manner to (2S,4R)-1-[(2S)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from [(3R,8S)-3-[[tert-butyl(diphenyl)silyl]oxymethyl]-1,2,3,5,6,7-hexahydropyrrolizin-8-yl]methanol. (formic acid salt, white solid). LC/MS (ESI) m/z: 1079.9 [M+H]⁺. ¹H NMR (400 MHz, CD₃OD) δ 9.17-9.01 (m, 1H), 8.87 (s, 1H), 8.49 (br s, 1H), 7.67-7.59 (m, 1H), 7.49-7.31 (m, 5H), 7.31-7.27 (m, 1H), 7.21-7.12 (m, 1H), 7.03-6.95 (m, 1H), 6.02-5.88 (m, 1H), 5.03 (q, J=6.8 Hz, 1H), 4.77-4.61 (m, 3H), 4.57-4.39 (m, 4H), 4.38-4.21 (m, 2H), 4.03-4.00 (m, 2H), 3.96-3.72 (m, 3H), 3.70-3.43 (m, 3H), 3.29-3.24 (m, 1H), 3.20-3.04 (m, 1H), 2.51-2.44 (m, 3H), 2.40-2.13 (m, 6H), 2.11-1.78 (m, 10H), 1.57-1.44 (m, 3H), 1.10-0.98 (m, 3H), 0.94-0.79 (m, 6H).

Exemplary Synthesis (2S,4R)-1-[(2S)-2-[3-[[(3R,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydr oxy-N-[(1S)-1-[4-(4-methylthiazol-5-yl)phenyl]ethyl]pyrrolidine-2-carboxamide (Compo und 323

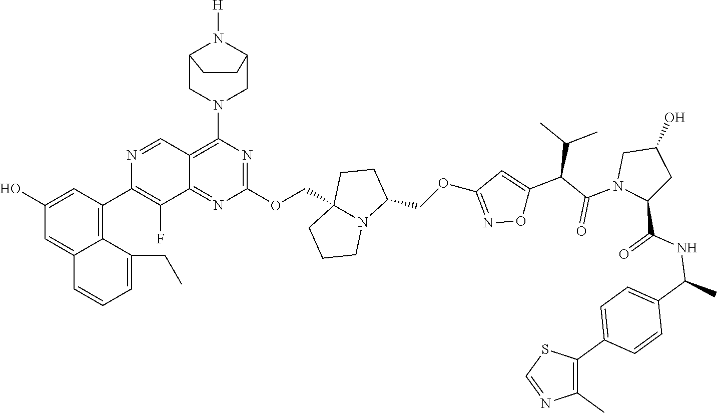 [SMILES: CCc1cccc2cc(O)cc(-c3ncc4c(N5CC6CCC(C5)N6)nc(OC[C@@]56CCCN5[C@@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compound was made in an analogous manner to (2S,4R)-1-[(2R)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from [(3R,8S)-3-[[tert-butyl(diphenyl)silyl]oxymethyl]-1,2,3,5,6,7-hexahydropyrrolizin-8-yl]methanol. (formic acid salt, white solid). LC/MS (ESI) m/z: 1079.9 [M+H]⁺. ¹H NMR (400 MHz, CD₃OD) δ 9.11-9.01 (m, 1H), 8.91-8.82 (m, 1H), 8.49 (d, J=3.6 Hz, 1H), 7.62 (d, J=8.0 Hz, 1H), 7.49-7.26 (m, 6H), 7.19-7.12 (m, 1H), 7.05-6.95 (m, 1H), 6.11-5.91 (m, 1H), 5.05-4.94 (m, 1H), 4.74-4.63 (m, 2H), 4.57 (t, J=8.0 Hz, 1H), 4.52-4.35 (m, 3H), 4.32-4.20 (m, 2H), 4.05-3.92 (m, 2H), 3.90-3.55 (m, 5H), 3.48-3.36 (m, 1H), 3.27-3.16 (m, 1H), 3.15-2.99 (m, 1H), 2.52-2.42 (m, 3H), 2.41-1.74 (m, 17H), 1.60-1.41 (m, 3H), 1.10-0.79 (m, 9H).

Exemplary Synthesis of (2S,4R)-1-[(2R)-2-[3-[[(3S,8R)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 311)

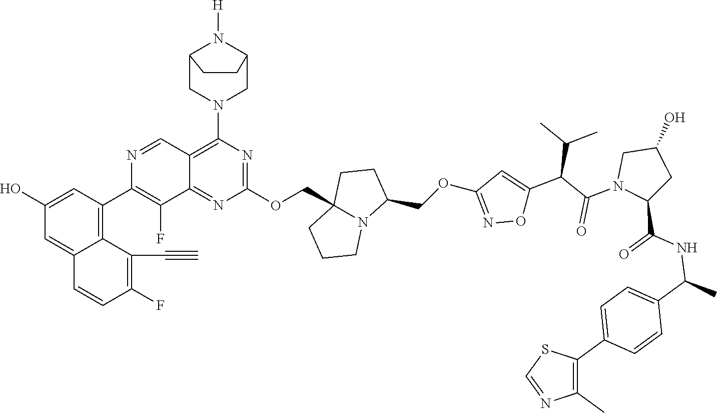 [SMILES: C#Cc1c(F)ccc2cc(O)cc(-c3ncc4c(N5CC6CCC(C5)N6)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compound was made in an analogous manner to (2S,4R)-1-[(2R)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate and (2S,4R)-4-[tert-butyl(diphenyl)silyl]oxy-1-[2-[3-[[(3R,8S)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide. (white solid). LC/MS (ESI) m/z: 1093.9 [M+H]⁺. ¹H NMR (400 MHz, CD₃OD) δ 9.05-8.98 (m, 1H), 8.88 (s, 1H), 7.87-7.83 (m, 1H), 7.48-7.28 (m, 6H), 7.25-7.21 (m, 1H), 6.01-5.92 (m, 1H), 5.03 (d, J=6.8 Hz, 1H), 4.60 (s, 3H), 4.50 (t, J=8.0 Hz, 1H), 4.44-4.38 (m, 1H), 4.33-4.22 (m, 2H), 4.18-4.11 (m, 2H), 3.83-3.60 (m, 7H), 3.23-3.13 (m, 1H), 3.10-3.02 (m, 1H), 2.90-2.82 (m, 1H), 2.50-2.45 (m, 3H), 2.40-2.30 (m, 1H), 2.26-2.15 (m, 2H), 2.11-2.05 (m, 1H), 1.96-1.72 (m, 11H), 1.58-1.50 (m, 3H), 1.08-1.01 (m, 3H), 0.91-0.85 (m, 3H).

Exemplary Synthesis of (2S,4R)-1-[(2S)-2-[3-[[(3S,8R)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 312)

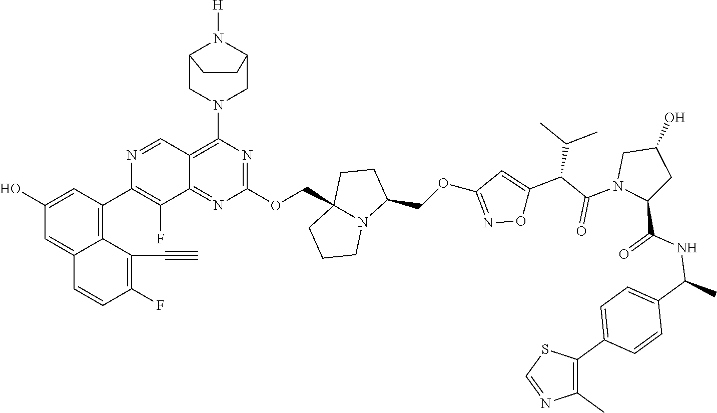 [SMILES: C#Cc1c(F)ccc2cc(O)cc(-c3ncc4c(N5CC6CCC(C5)N6)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was made in an analogous manner to (2S,4R)-1-[(2S)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[8-fluoro-7-[7-fluoro-3-(methoxymethoxy)-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate and (2S,4R)-4-[tert-butyl(diphenyl)silyl]oxy-1-[2-[3-[[(3R,8S)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide. (white solid). LC/MS (ESI) m/z: 1093.2 [M+H]⁺. ¹H NMR (400 MHz, CD₃OD) δ 9.01-8.96 (m, 1H), 8.89-8.83 (m, 1H), 7.86-7.83 (m, 1H), 7.46-7.27 (m, 6H), 7.23-7.20 (m, 1H), 6.04 (d, J=4.4 Hz, 1H), 5.04-4.96 (m, 1H), 4.68-4.54 (m, 4H), 4.41 (s, 1H), 4.30-4.19 (m, 2H), 4.16-4.07 (m, 2H), 3.78-3.61 (m, 7H), 3.18-3.11 (m, 1H), 3.06-3.03 (m, 1H), 2.86-2.77 (m, 1H), 2.50-2.43 (m, 3H), 2.38-2.34 (m, 1H), 2.24-2.19 (m, 2H), 2.08-2.02 (m, 1H), 1.94-1.64 (m, 11H), 1.47 (d, J=6.0 Hz, 3H), 1.04 (d, J=6.4 Hz, 3H), 0.94-0.86 (m, 3H).

Exemplary Synthesis of (2S,4R)-1-[(2S)-2-[3-[[(3S,8R)-8-[[4-(3,8-diazabicyclo[3.2.1]octan-3-yl)-7-(8-ethyl-1-naphth yl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 310) and (2S,4R)-1-[(2R)-2-[3-[[(3S,8R)-8-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 309)

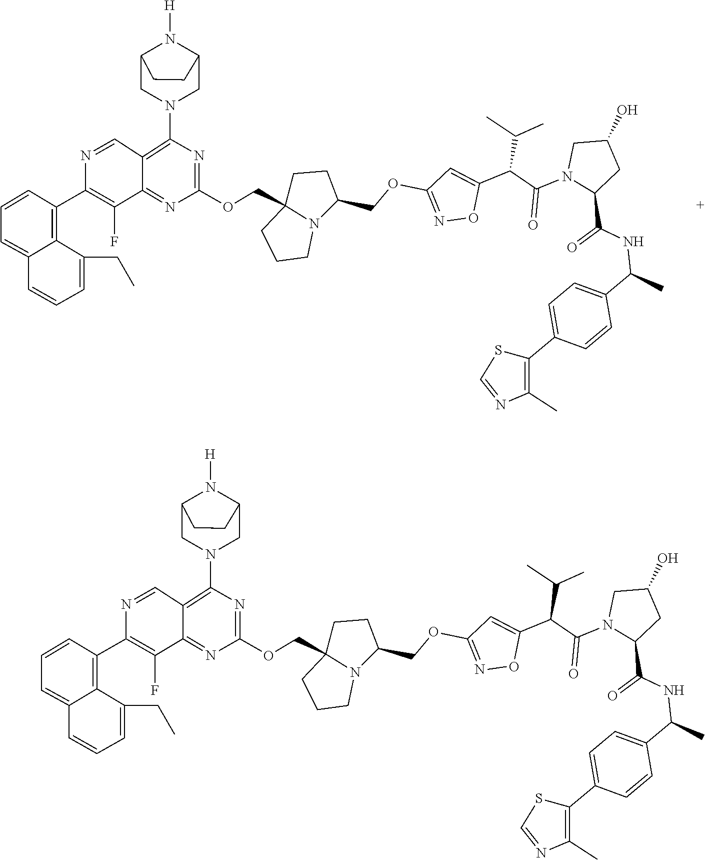 [SMILES: CCc1cccc2cccc(-c3ncc4c(N5CC6CCC(C5)N6)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12 ; CCc1cccc2cccc(-c3ncc4c(N5CC6CCC(C5)N6)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compounds were made in an analogous manner to (2S,4R)-1-[(2R)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide and (2S,4R)-1-[(2S)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(8-ethyl-1-naphthyl)-8-fluoro-2-methylsulfonyl-pyrido[4,3-d]pyrimidin-4-yl]-3,8-diazabicyclo[3.2.1]octane-8-carboxylate and (2S,4R)-4-[tert-butyl(diphenyl)silyl]oxy-1-[2-[3-[[(3S,8R)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrroledine-2-carboxamide. (2S,4R)-1-[(2S)-2-[3-[[(3S,8R)-8-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid) as white solid. LC/MS (ESI) m/z: 1063.9 [M+H]⁺. ¹H NMR (400 MHz, DMSO-d₆) δ 9.08 (d, J=2.8 Hz, 1H), 8.98 (s, 1H), 8.46-8.39 (m, 1H), 8.07 (d, J=8.0 Hz, 1H), 7.91 (d, J=8.0 Hz, 1H), 7.61-7.54 (m, 1H), 7.54-7.47 (m, 1H), 7.43 (d, J=7.6 Hz, 3H), 7.40-7.32 (m, 3H), 6.09-5.86 (m, 1H), 4.96-4.86 (m, 1H), 4.49-4.30 (m, 3H), 4.27 (s, 1H), 4.15-4.08 (m, 1H), 4.06-3.98 (m, 3H), 3.72-3.50 (m, 6H), 3.42 (d, J=10.4 Hz, 1H), 3.10-3.02 (m, 1H), 2.93 (dd, J=9.6, 3.6 Hz, 1H), 2.74-2.66 (m, 1H), 2.46-2.43 (m, 3H), 2.42-2.13 (m, 4H), 2.08-1.96 (m, 3H), 1.85-1.55 (m, 12H), 1.43-1.34 (m, 3H), 0.97-0.91 (m, 3H), 0.85-0.76 (m, 6H).

(2S,4R)-1-[(2R)-2-[3-[[(3S,8R)-8-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]meth oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). LC/MS (ESI) m/z: 1063.9 [M+H]⁺. ¹H NMR (400 MHz, DMSO-d₆) δ 9.07 (s, 1H), 9.01-8.90 (m, 1H), 8.28 (d, J=7.6 Hz, 1H), 8.07 (d, J=8.0 Hz, 1H), 7.90 (d, J=8.0 Hz, 1H), 7.57 (t, J=7.6 Hz, 1H), 7.53-7.26 (m, 7H), 6.17-6.03 (m, 1H), 5.00-4.81 (m, 1H), 4.54-4.33 (m, 3H), 4.25 (s, 1H), 4.15-3.96 (m, 4H), 3.74 (d, J=8.4 Hz, 1H), 3.68-3.45 (m, 6H), 3.08-2.98 (m, 1H), 2.95-2.84 (m, 1H), 2.72-2.62 (m, 1H), 2.46-2.43 (m, 3H), 2.42-2.15 (m, 4H), 2.13-1.86 (m, 4H), 1.83-1.71 (m, 5H), 1.67-1.56 (m, 6H), 1.33 (d, J=7.2 Hz, 3H), 0.95 (d, J=6.4 Hz, 3H), 0.85-0.79 (m, 6H).

Exemplary Synthesis of (2S,4R)-1-[(2R)-2-[3-[[(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 317) and (2S,4R)-1-[(2S)-2-[3-[[(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 316)

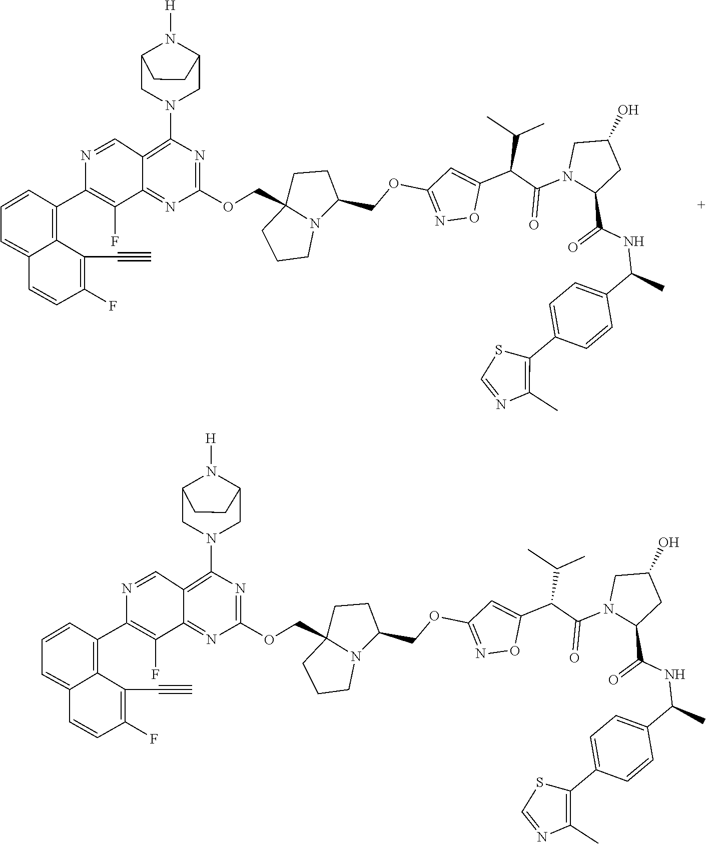 [SMILES: C#Cc1c(F)ccc2cccc(-c3ncc4c(N5CC6CCC(C5)N6)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12 ; C#Cc1c(F)ccc2cccc(-c3ncc4c(N5CC6CCC(C5)N6)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compounds were made in an analogous manner to (2S,4R)-1-[(2R)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide and (2S,4R)-1-[(2S)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[8-fluoro-7-[7-fluoro-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate and (2S,4R)-4-[tert-butyl(di phenyl)silyl]oxy-1-[2-[3-[[(3S,8R)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide. (2S,4R)-1-[(2R)-2-[3-[[(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). LC/MS (ESI) m/z: 1077.1 [M+H]⁺. ¹H NMR (400 MHz, CD₃OD) δ 9.02 (d, J=12.4 Hz, 1H), 8.88 (s, 1H), 8.17-8.06 (m, 2H), 7.71-7.61 (m, 2H), 7.51-7.32 (m, 5H), 6.01-5.92 (m, 1H), 5.03 (q, J=6.8 Hz, 1H), 4.60-4.55 (m, 1H), 4.51 (t, J=8.4 Hz, 1H), 4.45-4.37 (m, 1H), 4.34-4.20 (m, 2H), 4.19-4.07 (m, 2H), 3.86-3.55 (m, 7H), 3.50-3.40 (m, 1H), 3.24-3.14 (m, 1H), 3.09-2.98 (m, 1H), 2.92-2.79 (m, 1H), 2.52-2.44 (m, 3H), 2.42-2.31 (m, 1H), 2.26-2.13 (m, 2H), 2.12-2.04 (m, 1H), 1.98-1.65 (m, 12H), 1.60-1.47 (m, 3H), 1.08-1.01 (m, 3H), 0.93-0.82 (m, 3H). (2S,4R)-1-[(2S)-2-[3-[[(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). LC/MS (ESI) m/z: 1077.1 [M+H]⁺. ¹H NMR (400 MHz, CD₃OD) δ 9.00 (d, J=2.0 Hz, 1H), 8.91-8.83 (m, 1H), 8.16-8.05 (m, 2H), 7.71-7.60 (m, 2H), 7.49-7.31 (m, 5H), 6.09-5.94 (m, 1H), 4.98 (q, J=6.8 Hz, 1H), 4.60-4.53 (m, 2H), 4.45-4.38 (m, 1H), 4.31-4.18 (m, 2H), 4.17-4.05 (m, 2H), 3.82-3.58 (m, 7H), 3.45 (d, J=9.2 Hz, 1H), 3.21-3.10 (m, 1H), 3.08-2.97 (m, 1H), 2.91-2.76 (m, 1H), 2.52-2.44 (m, 3H), 2.42-2.31 (m, 1H), 2.26-2.16 (m, 2H), 2.10-2.01 (m, 1H), 1.99-1.62 (m, 12H), 1.58-1.42 (m, 3H), 1.08-0.93 (m, 3H), 0.92-0.81 (m, 3H).

Exemplary Synthesis of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 300)

Step 1: Preparation of [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy-tert-butyl-diphenyl-silane

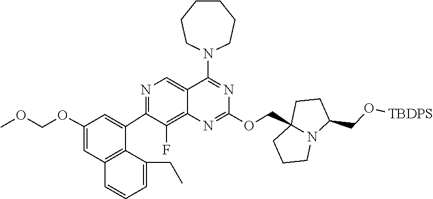

To a solution of 4-(azepan-1-yl)-7-[8-ethyl-3-(methoxymethoxy)-1-naphthyl]-8-fluoro-2-methylsulfonyl-pyrido[4,3-d]pyrimidine (505 mg, 0.937 mmol, 1.2 eq) and [(3S,8R)-3-[[tert-butyl(diphenyl)silyl]oxymethyl]-1,2,3,5,6,7-hexahydropyrrolizin-8-yl]methanol (320 mg, 0.781 mol, 1 eq) in toluene (10 mL) was added 4 Å MS (1.0 g), and the resulting mixture was stirred at 25° C. for 0.5 hour. t-BuONa (150 mg, 1.56 mmol, 2 eq) was then added at 0° C., and the reaction mixture was stirred at 0° C. for 0.5 hour. The reaction was quenched by addition of water/2N aqueous HCl (15 mL/0.7 mL) and extracted with EtOAc (15 mL×3). The combined organic extract was washed with brine (30 mL), dried over anhydrous Na₂SO₄, filtered, and concentrated under reduced pressure. The crude product was purified by flash chromatography on SiO₂ (gradient: 0˜30% tetrahydrofuran in petroleum ether) to afford [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy-tert-butyl-diphenyl-silane (528 mg, 0.608 mmol, 78% yield) as a yellow solid. LC/MS (ESI) m/z: 868.2 [M+H]⁺.

Step 2: Preparation of [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol

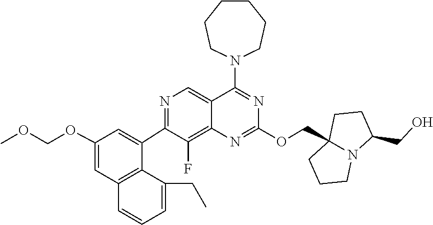 [SMILES: CCc1cccc2cc(OCOC)cc(-c3ncc4c(N5CCCCCC5)nc(OC[C@]56CCCN5[C@H](CO)CC6)nc4c3F)c12]

To a solution of [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy-tert-butyl-diphenyl-silane (528 mg, 0.608 mmol, 1 eq) in DMF (4 mL) was added CsF (739 mg, 4.87 mmol, 8 eq), and the reaction mixture was stirred at 25° C. for 40 hours. The reaction mixture was diluted CH₂Cl₂ (50 mL) and filtered. The filtrate was concentrated, and the crude product was purified by flash chromatography on SiO₂ (gradient: 0˜5% methanol (10% 7 M NH₃) in dichloromethane) to afford [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (255 mg, 0.405 mmol, 67% yield) as a yellow solid. LC/MS (ESI) m/z: 630.1 [M+H]⁺.

Step 3: Preparation of [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate

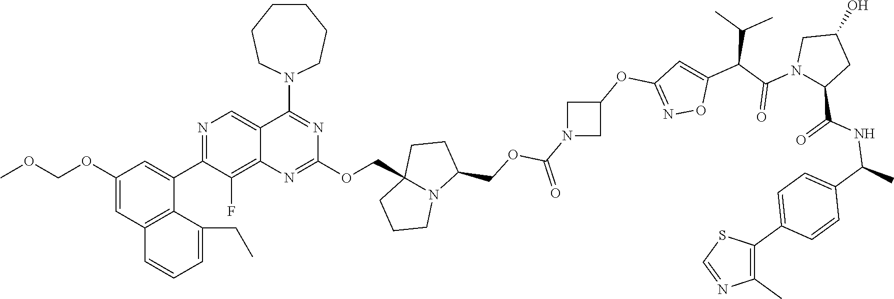 [SMILES: CCc1cccc2cc(OCOC)cc(-c3ncc4c(N5CCCCCC5)nc(OC[C@]56CCCN5[C@H](COC(=O)N5CC(Oc7cc([C@H](C(=O)N8C[C@H](O)C[C@H]8C(=O)N[C@@H](C)c8ccc(-c9scnc9C)cc8)C(C)C)on7)C5)CC6)nc4c3F)c12]

To a solution of [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol (125 mg, 0.198 mmol, 1 eq) in THF (5 mL) were added TEA (201 mg, 1.98 mmol, 0.276 mL, 10 eq), DMAP (2.4 mg, 0.020 mmol, 0.1 eq) and 4-nitrophenylcarbonochloridate (76 mg, 0.38 mmol, 1.9 eq), and the reaction mixture was stirred at 25° C. for 16 hours. (2S,4R)-1-[(2R)-2-[3-(Azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide (186 mg, 0.278 mmol, 1.4 eq, TFA) was then added, and the reaction mixture was stirred at 25° C. for 16 hours. The reaction mixture was filtered, and the filtrate was concentrated under reduced pressure. The crude product was purified by flash chromatography on SiO₂ (gradient: 0˜6% methanol in dichloromethane) to afford [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (174 mg, 0.144 mmol, 72% yield) as a white solid. LC/MS (ESI) m/z: 1209.1 [M+H]⁺.

Step 4: Preparation of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate

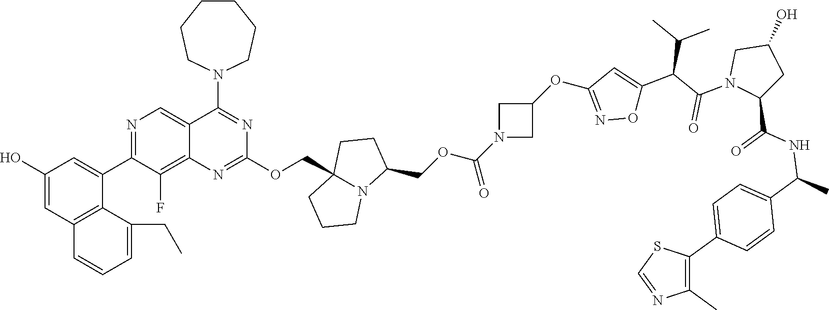 [SMILES: CCc1cccc2cc(O)cc(-c3ncc4c(N5CCCCCC5)nc(OC[C@]56CCCN5[C@H](COC(=O)N5CC(Oc7cc([C@H](C(=O)N8C[C@H](O)C[C@H]8C(=O)N[C@@H](C)c8ccc(-c9scnc9C)cc8)C(C)C)on7)C5)CC6)nc4c3F)c12]

To a solution of [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (174 mg, 0.144 mmol, 1 eq) in CH₂Cl₂ (3 mL) was added 4 M HCl/dioxane (1 mL), and the reaction mixture was stirred at 25° C. for 5 minutes. The reaction mixture was concentrated under reduced. To the resulting residue was added THF (30 mL) and TEA (0.2 mL), and the suspension was stirred at 20° C. for 0.5 hour. The mixture was filtered, and the filtrate was concentrated under reduced pressure. The crude product was purified by prep-HPLC (column: Phenomenex C18 75*30 mm*3 um; mobile phase: [8-58% CH₃CN in water (formic acid)]). Pure fractions were combined and concentrated under reduced pressure, then lyophilized to afford [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (79.5 mg, 0.066 mmol, 46% yield, formic acid salt) as a white solid. LC/MS (ESI) m/z: 1165.2 [M+H]⁺. ¹H NMR (400 MHz, CD₃OD) δ 9.22-9.12 (m, 1H), 8.90-8.84 (m, 1H), 8.49 (s, 1H), 7.66-7.59 (m, 1H), 7.48-7.26 (m, 6H), 7.20-7.12 (m, 1H), 7.04 (dd, J=13.2, 2.8 Hz, 1H), 6.05-5.93 (m, 1H), 5.12-5.06 (m, 1H), 5.05-4.99 (m, 1H), 4.66-4.56 (m, 2H), 4.53-4.38 (m, 4H), 4.37-4.22 (m, 3H), 4.20-4.05 (m, 5H), 4.02-3.88 (m, 2H), 3.81 (td, J=10.8, 4.4 Hz, 1H), 3.75-3.64 (m, 1H), 3.63-3.50 (m, 1H), 3.22-3.10 (m, 1H), 2.53-2.43 (m, 3H), 2.42-2.26 (m, 4H), 2.20-1.88 (m, 13H), 1.77-1.64 (m, 4H), 1.58-1.45 (m, 3H), 1.08-1.02 (m, 3H), 0.94-0.82 (m, 6H).

Exemplary Synthesis of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 305)

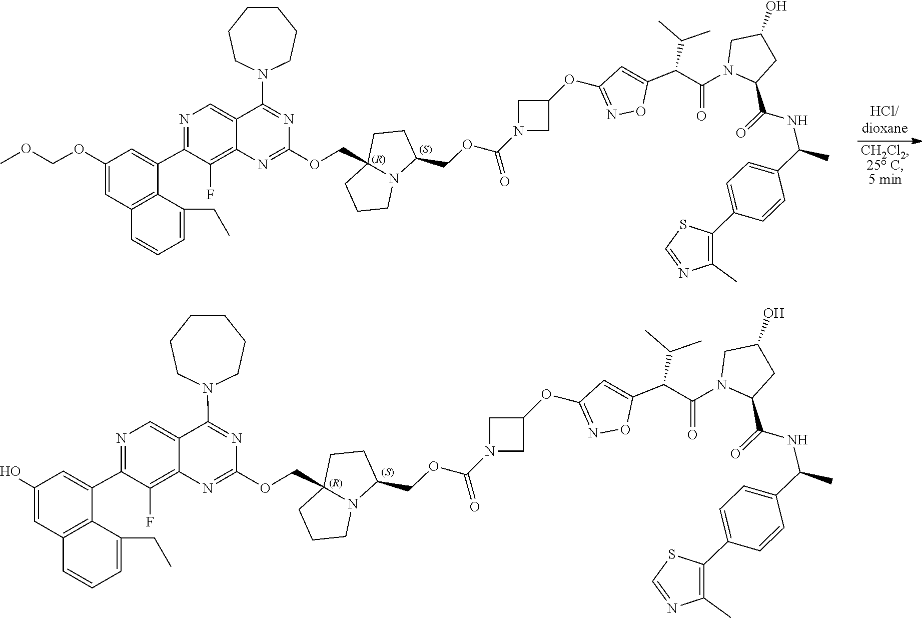

The title compound was made in an analogous manner to [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-1-[(2S)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1165.2 [M+H]⁺. ¹H NMR (400 MHz, CD₃OD) δ 9.18-9.10 (m, 1H), 8.85 (s, 1H), 8.49 (s, 1H), 7.62 (d, J=8.4 Hz, 1H), 7.49-7.25 (m, 6H), 7.15 (d, J=7.2 Hz, 1H), 7.03 (d, J=2.4 Hz, 1H), 6.06-5.98 (m, 1H), 5.16-5.03 (m, 1H), 4.98 (q, J=7.2 Hz, 1H), 4.57 (t, J=8.4 Hz, 1H), 4.52-4.38 (m, 3H), 4.35-4.17 (m, 3H), 4.16-4.03 (m, 5H), 4.01-3.84 (m, 2H), 3.76 (d, J=9.2 Hz, 1H), 3.73-3.61 (m, 2H), 3.46-3.32 (m, 2H), 3.20-3.07 (m, 1H), 2.52-2.43 (m, 3H), 2.42-2.18 (m, 5H), 2.15-1.84 (m, 12H), 1.77-1.64 (m, 4H), 1.59-1.43 (m, 3H), 1.09-0.94 (m, 3H), 0.94-0.80 (m, 6H).

Exemplary Synthesis of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate (Compound 286)

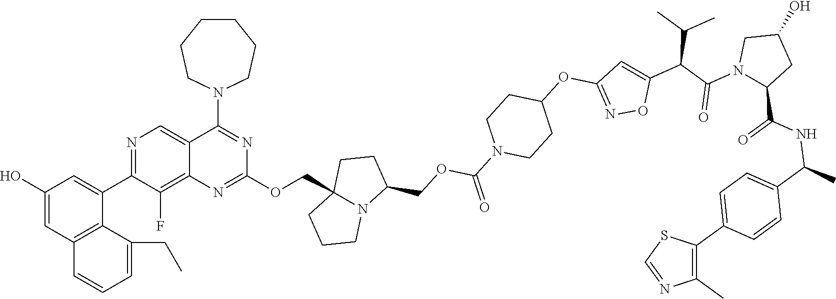 [SMILES: CCc1cccc2cc(O)cc(-c3ncc4c(N5CCCCCC5)nc(OC[C@]56CCCN5[C@H](COC(=O)N5CCC(Oc7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was made in an analogous manner to [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-4-hydroxy-1-[(2R)-3-methyl-2-[3-(4-piperidyloxy)isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1194.0 [M+H]⁺. ¹H NMR (CD₃OD, 400 MHz) δ 9.15 (d, J=5.2 Hz, 1H), 8.90-8.85 (m, 1H), 8.51 (s, 1H), 7.63 (d, J=8.0 Hz, 1H), 7.51-7.25 (m, 6H), 7.16 (dd, J=3.6, 6.4 Hz, 1H), 7.04 (dd, J=2.4, 6.4 Hz, 1H), 6.04-5.88 (m, 1H), 5.05-5.00 (m, 1H), 4.76-4.66 (m, 3H), 4.51 (t, J=8.0 Hz, 1H), 4.41 (d, J=6.4 Hz, 3H), 4.31-4.20 (m, 1H), 4.11 (d, J=4.8 Hz, 5H), 3.87-3.79 (m, 1H), 3.78-3.59 (m, 4H), 3.34 (d, J=1.6 Hz, 2H), 3.15-3.03 (m, 1H), 2.50-2.44 (m, 3H), 2.40-2.25 (m, 4H), 2.17-1.84 (m, 15H), 1.71 (s, 6H), 1.59-1.47 (m, 3H), 1.05 (d, J=6.4 Hz, 3H), 0.94-0.85 (m, 6H).

Exemplary Synthesis of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxypiperidine-1-carboxylate (Compound 282)

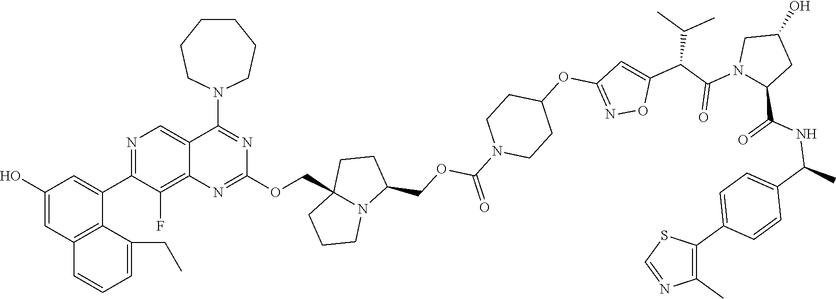 [SMILES: CCc1cccc2cc(O)cc(-c3ncc4c(N5CCCCCC5)nc(OC[C@]56CCCN5[C@H](COC(=O)N5CCC(Oc7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was made in an analogous manner to [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-4-hydroxy-1-[(2S)-3-methyl-2-[3-(4-piperidyloxy)isoxazol-5-yl]butanoyl]-N-[(1S)-1-[4-(4-methylthiazol-5-yl)phenyl]ethyl]pyrrolidine-2-carboxamide. (formic acid salt, yellow solid). LC/MS (ESI) m/z: 1081.2 [M+H]⁺. ¹H NMR (400 MHz, CDCl₃) δ 9.03 (d, J=13.2 Hz, 1H), 8.67 (s, 1H), 7.55-7.47 (m, 1H), 7.44-7.30 (m, 5H), 7.22-7.05 (m, 3H), 5.86 (s, 1H), 5.01-4.94 (m, 1H), 4.75-4.64 (m, 2H), 4.57-4.40 (m, 2H), 4.33-4.14 (m, 3H), 3.96 (s, 4H), 3.74-3.67 (m, 1H), 3.59-3.49 (m, 3H), 3.35-3.14 (m, 4H), 2.97-2.80 (m, 2H), 2.52-2.49 (m, 3H), 2.45-2.28 (m, 7H), 1.98 (s, 10H), 1.67 (s, 7H), 1.38-1.35 (m, 3H), 1.05-0.85 (m, 10H).

Exemplary Synthesis of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate (Compound 284)

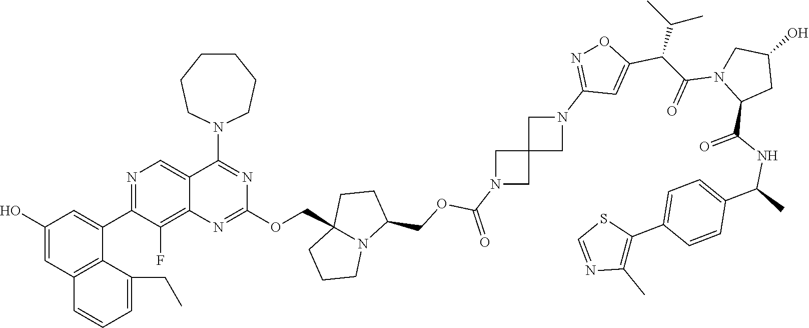 [SMILES: CCc1cccc2cc(O)cc(-c3ncc4c(N5CCCCCC5)nc(OC[C@]56CCCN5[C@H](COC(=O)N5CC7(C5)CN(c5cc([C@@H](C(=O)N8C[C@H](O)C[C@H]8C(=O)N[C@@H](C)c8ccc(-c9scnc9C)cc8)C(C)C)on5)C7)CC6)nc4c3F)c12]

The title compound was made in an analogous manner to [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-1-[(2S)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1190.6 [M+H]⁺. ¹H NMR (400 MHz, CD₃OD) δ 9.11 (s, 1H), 8.87 (s, 1H), 7.64-7.61 (m, 1H), 7.46-7.34 (m, 5H), 7.29-7.27 (m, 1H), 7.16-7.14 (m, 1H), 7.04-7.02 (m, 1H), 5.87 (s, 1H), 5.01-4.98 (m, 1H), 4.63 (s, 5H), 4.41-4.25 (m, 3H), 4.11-3.96 (m, 13H), 3.74-3.60 (m, 3H), 3.16-3.10 (m, 2H), 2.98-2.85 (m, 1H), 2.46 (s, 3H), 2.38-2.19 (m, 5H), 2.04-1.70 (m, 13H), 1.49-1.47 (m, 3H), 1.05-1.03 (m, 3H), 0.95-0.88 (m, 6H).

Exemplary Synthesis of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate (Compound 283)

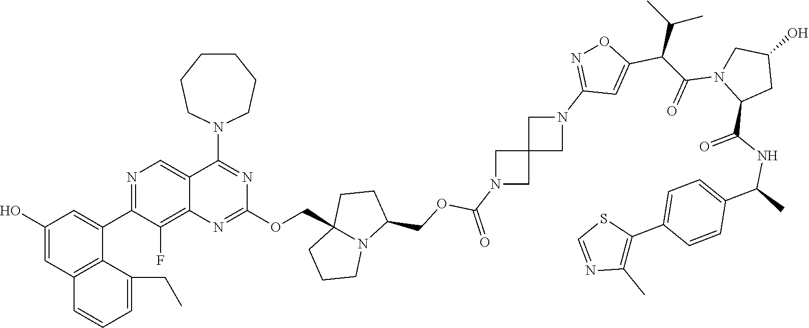 [SMILES: CCc1cccc2cc(O)cc(-c3ncc4c(N5CCCCCC5)nc(OC[C@]56CCCN5[C@H](COC(=O)N5CC7(C5)CN(c5cc([C@H](C(=O)N8C[C@H](O)C[C@H]8C(=O)N[C@@H](C)c8ccc(-c9scnc9C)cc8)C(C)C)on5)C7)CC6)nc4c3F)c12]

The title compound was made in an analogous manner to [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-1-[(2R)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1190.5 [M+H]⁺. ¹H NMR (CD₃OD, 400 MHz) δ 9.11 (s, 1H), 8.88 (s, 1H), 7.64-7.62 (m, 1H), 7.45-7.34 (m, 5H), 7.29-7.28 (m, 1H), 7.17-7.15 (m, 1H), 7.05-7.03 (m, 1H), 5.86-5.83 (m, 1H), 5.03-5.00 (m, 1H), 4.63 (s, 3H), 4.51-4.47 (m, 1H), 4.42-4.25 (m, 3H), 4.13-4.00 (m, 14H), 3.86-3.73 (m, 1H), 3.64-3.55 (m, 2H), 3.09 (s, 2H), 2.88 (m, 1H), 2.48 (s, 3H), 2.38-2.16 (m, 5H), 2.04-1.70 (m, 13H), 1.52-1.49 (m, 3H), 1.05-1.03 (m, 3H), 0.91-0.86 (m, 6H).

Exemplary Synthesis of [(3S,8R)-8-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 306)

Step 1: Preparation of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[3-chloro-2-cyclopropyl-5-(methoxymethoxy)phenyl]-8-fluoro-pyrido[4,3-d]pyrimidin-4-yl]-3,8-diazabicyclo[3.2.1]octane-8-carboxylate

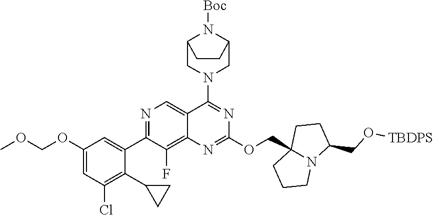

To a mixture of tert-butyl 3-[7-[3-chloro-2-cyclopropyl-5-(methoxymethoxy)phenyl]-8-fluoro-2-methylsulfonyl-pyrido[4,3-d]pyrimidin-4-yl]-3,8-diazabicyclo[3.2.1]octane-8-carboxylate (500 mg, 88% purity, 679 mmol, 1.1 eq), 4 Å molecular sieves (700 mg), and [(3S,8R)-3-[[tert-butyl(diphenyl)silyl]oxymethyl]-1,2,3,5,6,7-hexahydro pyrrolizin-8-yl]methanol (253 mg, 0.617 mmol, 1.0 eq) in toluene (16 mL) was added t-BuONa (148 mg, 1.54 mmol, 2.5 eq) at 0° C. under N₂, and the reaction mixture was stirred at 0° C. for 40 minutes. The mixture was treated with 2 M hydrochloric acid until pH ˜8 and diluted with water (20 mL). The organic phase was collected, and the aqueous phase was further extracted with ethyl acetate (3×30 mL). The combined organic extracts were washed with brine (5×10 mL), dried over anhydrous Na₂SO₄, filtered, and concentrated. The residue was purified by flash chromatography on SiO₂ (gradient: 0˜1% methanol in dichloromethane) to afford tert-butyl 3-[2-[[(3S,8R)-3-[[tert-butyl(diphenyl)silyl]oxymethyl]-1,2,3,5,6,7-hexahydropyrrolizin-8-yl]methoxy]-7-[3-chloro-2-cyclopropyl-5-(methoxymethoxy)phenyl]-8-fluoro-pyrido[4,3-d]pyrimidin-4-yl]-3,8-diazabicyclo[3.2.1]octane-8-carboxylate (546 mg, 0.424 mmol, 69% yield) as a yellow solid. LC/MS (ESI) m/z: 977.6 [M+H]⁺.

Step 2: Preparation of tert-butyl 3-[7-[3-chloro-2-cyclopropyl-5-(methoxymethoxy)phenyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

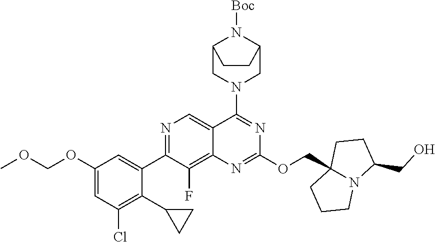 [SMILES: COCOc1cc(Cl)c(C2CC2)c(-c2ncc3c(N4CC5CCC(C4)N5C(=O)OC(C)(C)C)nc(OC[C@]45CCCN4[C@H](CO)CC5)nc3c2F)c1]

To a solution of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[3-chloro-2-cyclopropyl-5-(methoxymethoxy)phenyl]-8-fluoro-pyrido[4,3-d]pyrimidin-4-yl]-3,8-diazabicyclo[3.2.1]octane-8-carboxylate (1.1 g, 76% purity, 0.854 mmol, 1.0 eq) in DMF (20 mL) at 30° C. was added CsF (1.4 g, 9.00 mmol, 10.5 eq), and the reaction mixture was stirred at 30° C. for 12 hours. The reaction mixture was diluted with water (30 mL) and extracted with ethyl acetate (3×40 mL). The combined organic extracts were washed with brine (4×15 mL), dried over anhydrous Na₂SO₄, filtered, and concentrated. The residue was purified by flash chromatography on SiO₂ (gradient: 0˜7% methanol in dichloromethane) to afford tert-butyl 3-[7-[3-chloro-2-cyclopropyl-5-(methoxymethoxy)phenyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (542 mg, 92% purity, 0.675 mmol, 79.04% yield) as yellow solid. LC/MS (ESI) m/z: 739.3 [M+H]⁺.

Step 3: Preparation of tert-butyl 3-[7-[3-chloro-2-cyclopropyl-5-(methoxymethoxy)phenyl]-8-fluoro-2-[[(3S,8R)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate

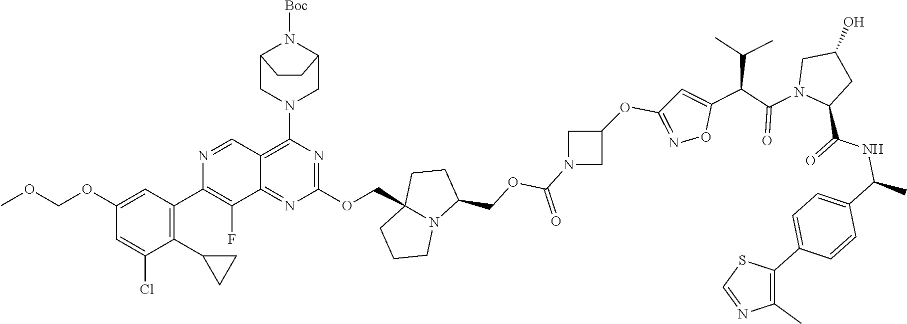 [SMILES: COCOc1cc(Cl)c(C2CC2)c(-c2ncc3c(N4CC5CCC(C4)N5C(=O)OC(C)(C)C)nc(OC[C@]45CCCN4[C@H](COC(=O)N4CC(Oc6cc([C@H](C(=O)N7C[C@H](O)C[C@H]7C(=O)N[C@@H](C)c7ccc(-c8scnc8C)cc7)C(C)C)on6)C4)CC5)nc3c2F)c1]

To tert-butyl 3-[7-[3-chloro-2-cyclopropyl-5-(methoxymethoxy)phenyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (160 mg, 0.216 mmol, 1.0 eq) in tetrahydrofuran (6 mL) were added triethylamine (0.241 mL, d=0.727 g/mL, 1.73 mmol, 8.0 eq), DMAP (8 mg, 0.065 mmol, 0.3 eq), and (4-nitrophenyl) carbonochloridate (65 mg, 0.325 mmol, 1.5 eq), and the resulting suspension was stirred at 28° C. for 15 hours. Triethylamine (0.151 mL, d=0.727 g/mL, 1.08 mmol, 5.0 eq) and (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide (202 mg TFA salt, 0.303 mmol, 1.4 eq) were added, and the reaction mixture was stirred at 28° C. for 1 hour. The reaction mixture was filtered and concentrated, and the resulting residue was purified by flash chromatography on SiO₂ (gradient: 0˜10% methanol in dichloromethane) to afford tert-butyl 3-[7-[3-chloro-2-cyclopropyl-5-(methoxymethoxy)phenyl]-8-fluoro-2-[[(3S,8R)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (280 mg) as a yellow oil. LC/MS (ESI) m/z: 1318.3 [M+H]⁺.

Step 4: Preparation of [(3S,8R)-8-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate

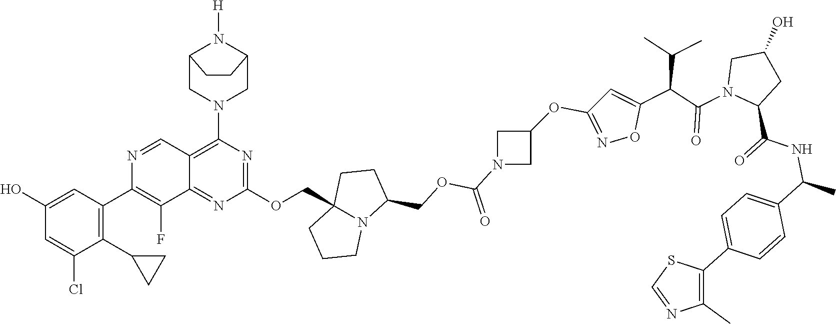 [SMILES: Cc1ncsc1-c1ccc([C@H](C)NC(=O)[C@@H]2C[C@@H](O)CN2C(=O)[C@@H](c2cc(OC3CN(C(=O)OC[C@@H]4CC[C@@]5(COc6nc(N7CC8CCC(C7)N8)c7cnc(-c8cc(O)cc(Cl)c8C8CC8)c(F)c7n6)CCCN45)C3)no2)C(C)C)cc1]

To tert-butyl 3-[7-[3-chloro-2-cyclopropyl-5-(methoxymethoxy)phenyl]-8-fluoro-2-[[(3S,8R)-3-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (280 mg, 0.212 mmol, 1.0 eq) in dichloromethane (3 mL) was added TFA (1 mL, d=1.54 g/mL) in one portion at 25° C., and the reaction mixture was stirred at 25° C. for 40 minutes. The mixture was adjusted to pH=8 by addition of saturated aqueous NaHCO₃. The resulting suspension was extracted with dichloromethane/methanol (80 mL, v/v=10/1, and the combined organic extract was washed with brine (30 mL), dried over anhydrous Na₂SO₄, filtered, and concentrated. The crude residue was purified by preparative HPLC {column: Phenomenex C18 75*30 mm*3 um; mobile phase: [2-42% CH₃CN in water (formic acid)]}. Pure fractions were combined, then lyophilized to afford [(3S,8R)-8-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (104.0 mg, 0.084 mmol, 40% yield, formic acid salt) as a white solid. LC/MS (ESI) m/z: 1174.9 [M+H]⁺. ¹H NMR (400 MHz, CD₃OD) δ 9.10 (s, 1H), 8.90-8.85 (m, 1H), 8.49 (s, 1H), 7.50-7.33 (m, 4H), 7.01-6.94 (m, 1H), 6.81 (d, J=2.4 Hz, 1H), 6.09-5.92 (m, 1H), 5.16-5.08 (m, 1H), 5.03 (q, J=7.0 Hz, 1H), 4.78-4.67 (m, 3H), 4.52 (t, J=8.4 Hz, 1H), 4.47-4.38 (m, 3H), 4.34 (d, J=2.4 Hz, 2H), 4.23 (dd, J=4.0, 11.6 Hz, 1H), 4.15-4.06 (m, 1H), 3.99 (s, 4H), 3.91-3.79 (m, 3H), 3.69 (d, J=10.0 Hz, 1H), 3.62 (d, J=11.2 Hz, 1H), 3.35 (s, 1H), 3.11 (d, J=5.6 Hz, 1H), 2.51-2.45 (m, 3H), 2.41-2.28 (m, 2H), 2.24-2.16 (m, 1H), 2.13-2.08 (m, 1H), 2.06-1.82 (m, 12H), 1.59-1.48 (m, 3H), 1.05 (d, J=6.4 Hz, 3H), 0.93-0.84 (m, 3H), 0.62 (d, J=6.0 Hz, 2H), 0.06 (s, 2H).

Exemplary Synthesis of [(3S,8R)-8-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]oxyazetidine-1-carboxylate (Compound 322)

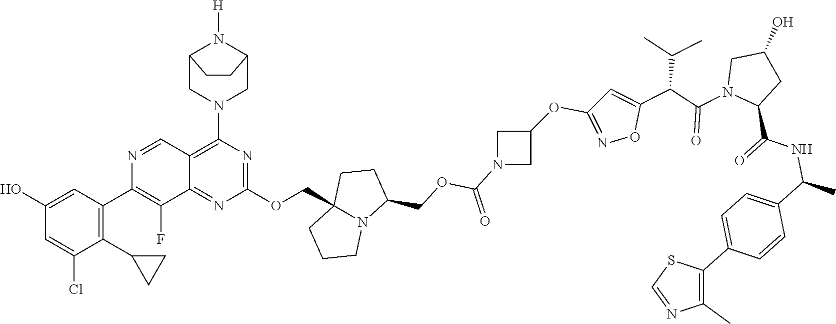 [SMILES: Cc1ncsc1-c1ccc([C@H](C)NC(=O)[C@@H]2C[C@@H](O)CN2C(=O)[C@H](c2cc(OC3CN(C(=O)OC[C@@H]4CC[C@@]5(COc6nc(N7CC8CCC(C7)N8)c7cnc(-c8cc(O)cc(Cl)c8C8CC8)c(F)c7n6)CCCN45)C3)no2)C(C)C)cc1]

The title compound was made in an analogous manner to [(3S,8R)-8-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from tert-butyl 3-[7-[3-chloro-2-cyclopropyl-5-(methoxymethoxy)phenyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-1-[(2S)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1174.8 [M+H]⁺. ¹H NMR (400 MHz, CD₃OD) δ 9.10 (s, 1H), 8.91-8.83 (m, 1H), 8.44 (s, 2H), 7.45-7.33 (m, 4H), 6.97 (d, J=2.4 Hz, 1H), 6.80 (d, J=2.4 Hz, 1H), 6.06-6.00 (m, 1H), 5.12-5.05 (m, 1H), 5.02-4.97 (m, 1H), 4.76 (d, J=13.6 Hz, 2H), 4.58 (t, J=8.0 Hz, 1H), 4.48 (s, 2H), 4.42 (s, 1H), 4.36-4.23 (m, 3H), 4.16-4.09 (m, 1H), 4.08-4.02 (m, 2H), 4.01-3.91 (m, 2H), 3.89-3.82 (m, 2H), 3.81-3.76 (m, 1H), 3.74-3.64 (m, 2H), 3.50-3.40 (m, 1H), 3.39-3.34 (m, 1H), 3.18 (d, J=5.2 Hz, 1H), 2.49-2.45 (m, 3H), 2.41-2.31 (m, 2H), 2.27-2.20 (m, 1H), 2.18-2.10 (m, 2H), 2.07-1.94 (m, 10H), 1.89-1.81 (m, 1H), 1.48 (d, J=7.2 Hz, 3H), 1.05 (d, J=6.4 Hz, 3H), 0.90 (d, J=6.4 Hz, 3H), 0.67-0.57 (m, 2H), 0.06 (d, J=1.6 Hz, 2H).

Exemplary Synthesis of [(3S,8R)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methyl 3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 303)

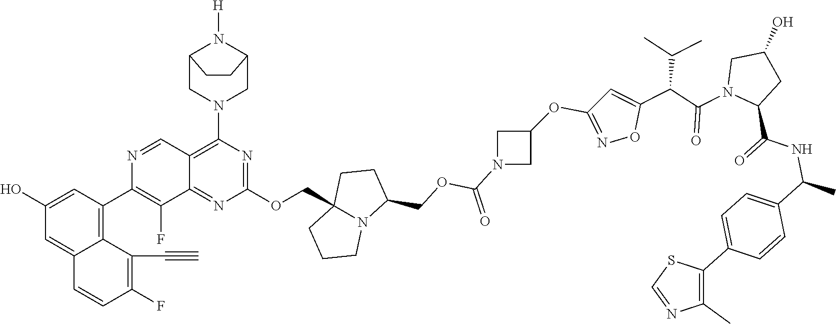 [SMILES: C#Cc1c(F)ccc2cc(O)cc(-c3ncc4c(N5CC6CCC(C5)N6)nc(OC[C@]56CCCN5[C@H](COC(=O)N5CC(Oc7cc([C@@H](C(=O)N8C[C@H](O)C[C@H]8C(=O)N[C@@H](C)c8ccc(-c9scnc9C)cc8)C(C)C)on7)C5)CC6)nc4c3F)c12]

The title compound was made in an analogous manner to [(3S,8R)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethynyl-7-fluoro-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-1-[(2S)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (light yellow solid). LC/MS (ESI) m/z: 1192.1 [M+H]⁺. ¹H NMR (400 MHz, CD₃OD) δ 8.99 (s, 1H), 8.86 (s, 1H), 7.86-7.82 (m, 1H), 7.43-7.30 (m, 6H), 7.22-7.20 (m, 1H), 6.04 (s, 1H), 5.01-4.97 (m, 1H), 4.62-4.55 (m, 3H), 4.42-4.17 (m, 5H), 4.06-3.92 (m, 4H), 3.78-3.65 (m, 7H), 3.36-3.34 (m, 1H), 3.04-3.01 (m, 2H), 2.81-2.77 (m, 1H), 2.46 (s, 3H), 2.39-2.34 (m, 1H), 2.21-2.19 (m, 3H), 1.98-1.73 (m, 12H), 1.49-1.47 (m, 3H), 1.06-1.04 (m, 3H), 0.91-0.89 (m, 3H).

Exemplary Synthesis of [(3S,8R)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 292)

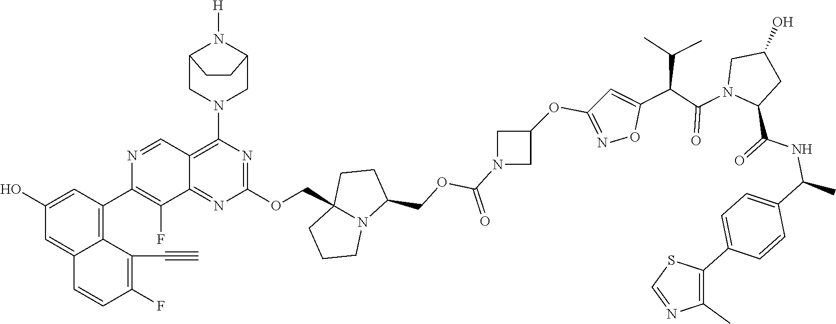 [SMILES: C#Cc1c(F)ccc2cc(O)cc(-c3ncc4c(N5CC6CCC(C5)N6)nc(OC[C@]56CCCN5[C@H](COC(=O)N5CC(Oc7cc([C@H](C(=O)N8C[C@H](O)C[C@H]8C(=O)N[C@@H](C)c8ccc(-c9scnc9C)cc8)C(C)C)on7)C5)CC6)nc4c3F)c12]

The title compound was made in an analogous manner to [(3S,8R)-8-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethynyl-7-fluoro-3-(methoxymethoxy)-1-naphthyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (yellow solid). LC/MS (ESI) m/z: 1192.9 [M+H]⁺. ¹H NMR (400 MHz, CD₃OD) δ 9.01 (d, J=2.0 Hz, 1H), 8.89-8.85 (m, 1H), 7.85 (d, J=5.6, 8.9 Hz, 1H), 7.46-7.37 (m, 4H), 7.36-7.33 (m, 1H), 7.36-7.28 (m, 1H), 7.22 (d, J=2.4 Hz, 1H), 6.04-5.94 (m, 1H), 5.19-5.10 (m, 1H), 5.03 (q, J=6.8 Hz, 1H), 4.67-4.55 (m, 3H), 4.51 (t, J=8.2 Hz, 1H), 4.45-4.32 (m, 3H), 4.26-4.22 (m, 1H), 4.10-4.05 (m, 1H), 4.04-3.92 (m, 3H), 3.82 (dd, J=4.0, 10.8 Hz, 1H), 3.75-3.71 (m, 1H), 3.68 (d, J=9.2 Hz, 3H), 3.61 (d, J=11.2 Hz, 1H), 3.38-3.34 (m, 2H), 3.04 (s, 2H), 2.82 (d, J=4.8 Hz, 1H), 2.49-2.45 (m, 3H), 2.41-2.32 (m, 1H), 2.24-2.14 (m, 2H), 2.04-1.73 (m, 12H), 1.58-1.49 (m, 3H), 1.05 (d, J=6.4 Hz, 3H), 0.92-0.85 (m, 3H).

Exemplary Synthesis of [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 294)

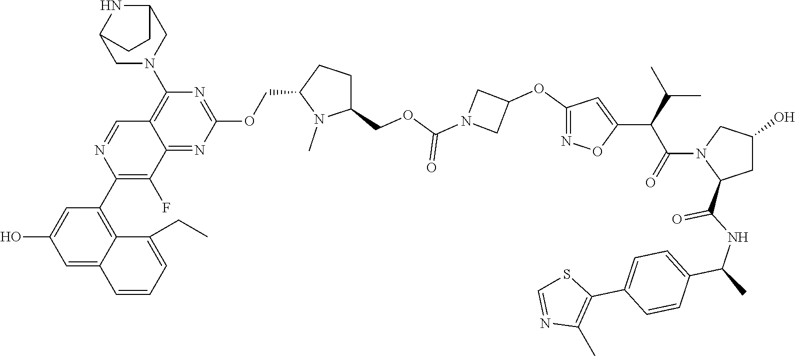 [SMILES: CCc1cccc2cc(O)cc(-c3ncc4c(N5CC6CCC(C5)N6)nc(OC[C@@H]5CC[C@@H](COC(=O)N6CC(Oc7cc([C@H](C(=O)N8C[C@H](O)C[C@H]8C(=O)N[C@@H](C)c8ccc(-c9scnc9C)cc8)C(C)C)on7)C6)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid).

LC/MS (ESI) m/z: 1152.9 [M+H]⁺. ¹H NMR (400 MHz, CD₃OD) δ 9.15-9.05 (m, 1H), 8.88 (s, 1H), 8.42 (s, 1H), 7.63 (d, J=8.0 Hz, 1H), 7.46-7.34 (m, 5H), 7.31-7.28 (m, 1H), 7.17 (d, J=7.2 Hz, 1H), 7.06-6.97 (m, 1H), 6.04 (d, J=2.0 Hz, 1H), 5.19-5.13 (m, 1H), 4.61 (d, J=4.4 Hz, 6H), 4.43-4.33 (m, 3H), 4.27-4.14 (m, 2H), 4.03-4.02 (m, 4H), 3.92-3.79 (m, 3H), 3.70-3.44 (m, 5H), 2.76-2.68 (m, 3H), 2.51-2.44 (m, 3H), 2.41-2.28 (m, 3H), 2.23-2.12 (m, 3H), 2.08-2.01 (m, 3H), 1.95-1.93 (m, 1H), 1.92-1.86 (m, 1H), 1.84-1.74 (m, 1H), 1.57-1.44 (m, 3H), 1.04 (d, J=6.4 Hz, 3H), 0.93-0.85 (m, 6H).

Exemplary Synthesis of [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 291)

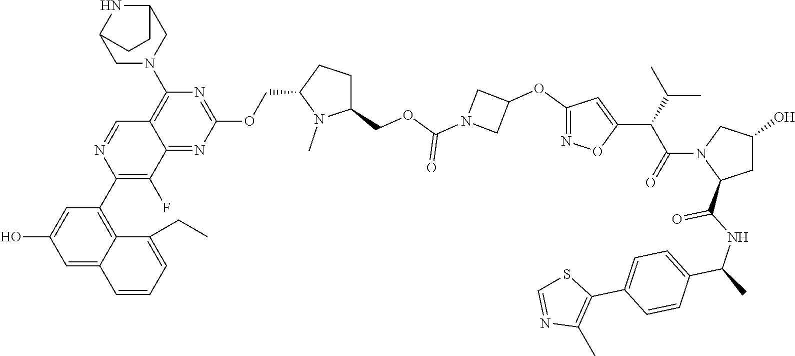 [SMILES: CCc1cccc2cc(O)cc(-c3ncc4c(N5CC6CCC(C5)N6)nc(OC[C@@H]5CC[C@@H](COC(=O)N6CC(Oc7cc([C@@H](C(=O)N8C[C@H](O)C[C@H]8C(=O)N[C@@H](C)c8ccc(-c9scnc9C)cc8)C(C)C)on7)C6)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-1-[(2S)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1152.2 [M+H]⁺. ¹H NMR (CD₃OD, 400 MHz) δ 9.07 (s, 1H), 8.86 (s, 1H), 8.49 (s, 1H), 7.62 (d, J=8.0 Hz, 1H), 7.47-7.32 (m, 5H), 7.29 (d, J=2.4 Hz, 1H), 7.15 (d, J=6.8 Hz, 1H), 7.02-6.99 (m, 1H), 6.07-6.02 (m, 1H), 5.13-5.07 (m, 1H), 5.01-4.97 (m, 1H), 4.81-4.69 (m, 2H), 4.64-4.54 (m, 3H), 4.41-4.22 (m, 4H), 4.19-4.12 (m, 1H), 4.06-3.75 (m, 7H), 3.73-3.62 (m, 2H), 3.58-3.52 (m, 1H), 3.49-3.42 (m, 1H), 2.70 (s, 3H), 2.50-2.44 (m, 3H), 2.41-2.14 (m, 6H), 2.05-1.73 (m, 7H), 1.60-1.44 (m, 3H), 1.09-0.93 (m, 3H), 0.92-0.80 (m, 6H).

Exemplary Synthesis of [(2S,5S)-5-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1-methyl-pyrrolidin-2-yl]methyl 3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 287)

Step 1: Preparation of tert-butyl 3-[2-[[(2S,5S)-5-[[tert-butyl(diphenyl)silyl]oxymethyl]-1-methyl-pyrrolidin-2-yl]methoxy]-7-[3-chloro-2-cyclopropyl-5-(methoxymethoxy)phenyl]-8-fluoro-pyrido[4,3-d]pyrimidin-4-yl]-3,8-diazabicyclo[3.2.1]octane-8-carboxylate

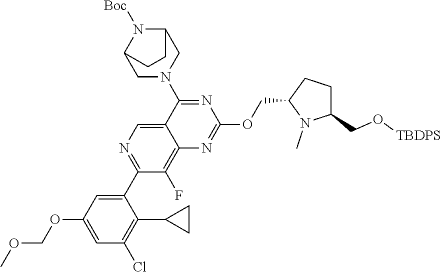

To a mixture of tert-butyl 3-[7-[3-chloro-2-cyclopropyl-5-(methoxymethoxy)phenyl]-8-fluoro-2-methylsulfonyl-pyrido[4,3-d]pyrimidin-4-yl]-3,8-diazabicyclo[3.2.1]octane-8-carboxylate (1.30 g, 2.01 mmol, 1.0 eq), 4 Å molecular sieves (1.5 g), and [(2S,5S)-5-[[tert-butyl(diphenyl)silyl]oxymethyl]-1-methyl-pyrrolidin-2-yl]methanol (769 mg, 2.01 mmol, 1.0 eq) in toluene (30 mL) at 0° C. under N₂ was added t-BuONa (482 mg, 5.01 mmol, 2.5 eq), and the reaction mixture was stirred at 0° C. for 40 minutes. The mixture was treated with 2 M hydrochloric acid until pH ˜8 and diluted with water (30 mL). The organic phase was separated, and the aqueous phase was further extracted with ethyl acetate (3×40 mL). The combined organic extracts were washed with brine (30 mL), dried over anhydrous Na₂SO₄, filtered, and concentrated. The residue was purified by flash chromatography on SiO₂ (gradient: 0˜42% ethyl acetate in petroleum ether) to afford tert-butyl 3-[2-[[(2S,5S)-5-[[tert-butyl(diphenyl)silyl]oxymethyl]-1-methyl-pyrrolidin-2-yl]methoxy]-7-[3-chloro-2-cyclopropyl-5-(methoxymethoxy)phenyl]-8-fluoro-pyrido[4,3-d]pyrimidin-4-yl]-3,8-diazabicyclo[3.2.1]octane-8-carboxylate (1.50 g, 1.40 mmol, 70% yield) as a yellow solid. LC/MS (ESI) m/z: 951.2 [M+H]⁺.

Step 2: Preparation of tert-butyl 3-[7-[3-chloro-2-cyclopropyl-5-(methoxymethoxy)phenyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate

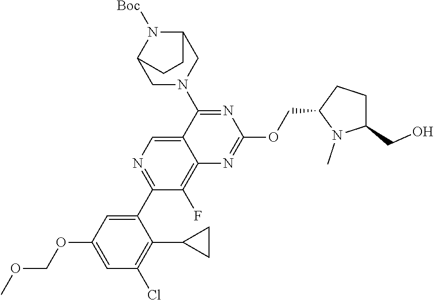 [SMILES: COCOc1cc(Cl)c(C2CC2)c(-c2ncc3c(N4CC5CCC(C4)N5C(=O)OC(C)(C)C)nc(OC[C@@H]4CC[C@@H](CO)N4C)nc3c2F)c1]

To a solution of tert-butyl 3-[2-[[(2S,5S)-5-[[tert-butyl(diphenyl)silyl]oxymethyl]-1-methyl-pyrrolidin-2-yl]methoxy]-7-[3-chloro-2-cyclopropyl-5-(methoxymethoxy)phenyl]-8-fluoro-pyrido[4,3-d]pyrimidin-4-yl]-3,8-diazabicyclo[3.2.1]octane-8-carboxylate (1.50 g, 89% purity, 1.40 mmol, 1.0 eq) in DMF (30 mL) at 30° C. was added CsF (1.92 g, 12.61 mmol, 8.0 eq), and the reaction mixture was stirred at 30° C. for 12 hours. The reaction mixture was diluted with water (40 mL) and extracted with ethyl acetate (3×40 mL). The combined organic extracts were washed with brine (5×20 mL), dried over anhydrous Na₂SO₄, filtered, and concentrated. The residue was purified by flash chromatography on SiO₂ (gradient: 0˜10% methanol in dichloromethane) afford tert-butyl 3-[7-[3-chloro-2-cyclopropyl-5-(methoxymethoxy)phenyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (888 mg, 1.20 mmol, 76% yield) as a yellow solid. LC/MS (ESI) m/z: 713.1 [M+H]⁺.

Step 3: Preparation of [(2S,5S)-5-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1-methyl-pyrrolidin-2-yl]methyl 3-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate

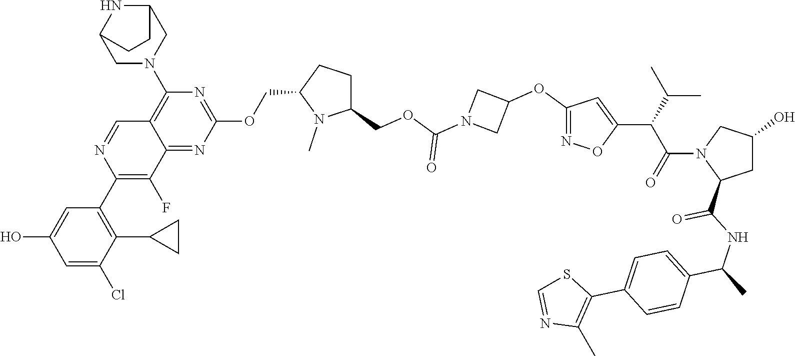 [SMILES: Cc1ncsc1-c1ccc([C@H](C)NC(=O)[C@@H]2C[C@@H](O)CN2C(=O)[C@H](c2cc(OC3CN(C(=O)OC[C@@H]4CC[C@@H](COc5nc(N6CC7CCC(C6)N7)c6cnc(-c7cc(O)cc(Cl)c7C7CC7)c(F)c6n5)N4C)C3)no2)C(C)C)cc1]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[3-chloro-2-cyclopropyl-5-(methoxymethoxy)phenyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-1-[(2S)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1148.9 [M+H]⁺. ¹H NMR (400 MHz, CD₃OD) δ 9.08 (s, 1H), 8.90-8.84 (m, 1H), 8.45 (s, 1H), 7.49-7.31 (m, 4H), 6.97 (d, J=2.4 Hz, 1H), 6.79 (d, J=2.4 Hz, 1H), 6.10-6.01 (m, 1H), 5.16-5.08 (m, 1H), 5.02-4.96 (m, 1H), 4.77-4.70 (m, 2H), 4.62-4.53 (m, 3H), 4.42 (s, 1H), 4.39-4.29 (m, 2H), 4.28-4.22 (m, 1H), 4.20-4.12 (m, 1H), 4.06-3.91 (m, 4H), 3.88-3.75 (m, 3H), 3.74-3.63 (m, 2H), 3.59-3.51 (m, 1H), 3.50-3.43 (m, 1H), 2.71 (s, 3H), 2.50-2.44 (m, 3H), 2.42-2.31 (m, 1H), 2.26-2.12 (m, 3H), 2.07-1.78 (m, 8H), 1.60-1.45 (m, 3H), 1.08-0.93 (m, 3H), 0.93-0.82 (m, 3H), 0.72-0.52 (m, 2H), 0.06 (s, 2H).

Exemplary Synthesis of [(2S,5S)-5-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1-methyl-pyrrolidin-2-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 285)

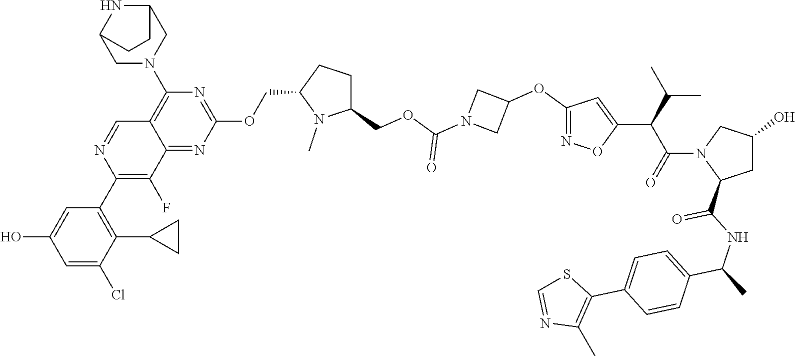 [SMILES: Cc1ncsc1-c1ccc([C@H](C)NC(=O)[C@@H]2C[C@@H](O)CN2C(=O)[C@@H](c2cc(OC3CN(C(=O)OC[C@@H]4CC[C@@H](COc5nc(N6CC7CCC(C6)N7)c6cnc(-c7cc(O)cc(Cl)c7C7CC7)c(F)c6n5)N4C)C3)no2)C(C)C)cc1]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[3-chloro-2-cyclopropyl-5-(methoxymethoxy)phenyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1148.5 [M+H]⁺. ¹HNMR (400 MHz, CD₃OD) δ 9.12-9.07 (m, 1H), 8.92-8.85 (m, 1H), 8.41 (s, 1H), 7.48-7.35 (m, 4H), 6.98 (d, J=2.4 Hz, 1H), 6.84-6.74 (m, 1H), 6.06 (s, 1H), 5.20-5.12 (m, 1H), 5.06-4.99 (m, 1H), 4.76 (t, J=10.8 Hz, 3H), 4.64-4.57 (m, 3H), 4.51 (t, J=8.0 Hz, 1H), 4.44 (d, J=2.0 Hz, 1H), 4.41-4.33 (m, 2H), 4.30-4.23 (m, 1H), 4.21-4.15 (m, 1H), 4.08-3.99 (m, 4H), 3.90-3.80 (m, 3H), 3.70 (d, J=9.6 Hz, 1H), 3.62 (d, J=11.2 Hz, 1H), 3.56-3.54 (m, 1H), 3.50-3.46 (m, 1H), 2.75-2.69 (m, 3H), 2.51-2.45 (m, 3H), 2.42-2.32 (m, 1H), 2.24-2.12 (m, 3H), 1.98-1.95 (m, 4H), 1.93-1.76 (m, 3H), 1.59-1.48 (m, 3H), 1.05 (d, J=6.4 Hz, 3H), 0.92-0.83 (m, 3H), 0.70-0.57 (m, 2H), 0.07 (d, J=0.8 Hz, 2H).

Exemplary Synthesis of [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl (3R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carboxylate (Compound 298)

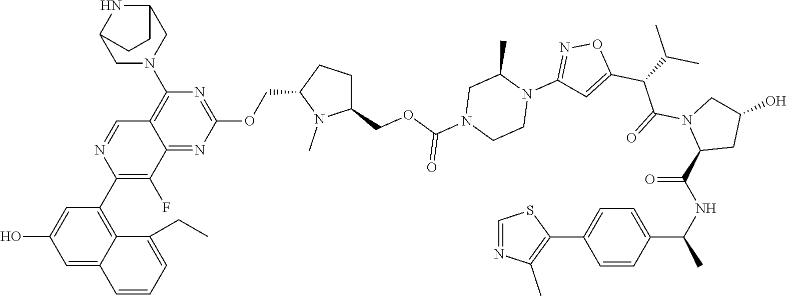 [SMILES: CCc1cccc2cc(O)cc(-c3ncc4c(N5CC6CCC(C5)N6)nc(OC[C@@H]5CC[C@@H](COC(=O)N6CCN(c7cc([C@@H](C(=O)N8C[C@H](O)C[C@H]8C(=O)N[C@@H](C)c8ccc(-c9scnc9C)cc8)C(C)C)on7)[C@H](C)C6)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-4-hydroxy-1-[(2S)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1180.0. [M+H]⁺. ¹H NMR (400 MHz, CD₃OD) δ 9.08 (s, 1H), 8.86 (s, 1H), 7.63 (d, J=8.0 Hz, 1H), 7.46-7.29 (m, 6H), 7.16 (d, J=8.0 Hz, 1H), 7.01-7.00 (m, 1H), 6.12-6.08 (m, 1H), 5.00-4.95 (m, 1H), 4.75-4.56 (m, 6H), 4.42 (s, 1H), 4.27 (s, 2H), 4.06-4.00 (m, 3H), 3.91-3.80 (m, 4H), 3.75-3.61 (m, 3H), 3.57-3.47 (m, 2H), 3.15-2.96 (m, 3H), 2.73 (s, 3H), 2.45 (s, 3H), 2.40-2.17 (m, 6H), 2.08-1.90 (m, 6H), 1.80 (s, 1H), 1.59-1.46 (m, 3H), 1.12-1.05 (m, 6H), 0.95-0.83 (m, 6H).

Exemplary Synthesis of [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl (3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carboxylate (Compound 301)

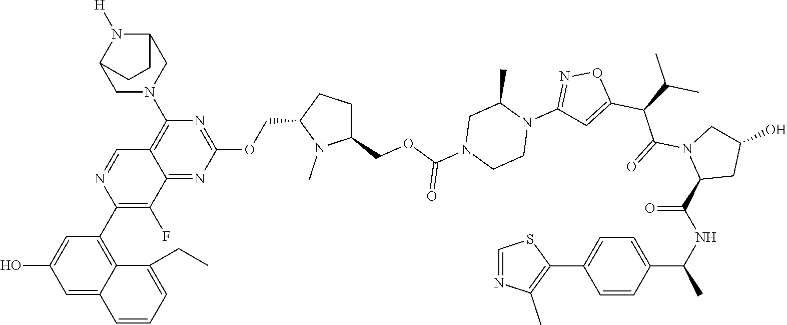 [SMILES: CCc1cccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)[C@H](C)C6)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1179.3 [M+H]⁺. ¹H NMR (400 MHz, CD₃OD) δ 9.09 (s, 1H), 8.89-8.84 (m, 1H), 8.49 (s, 1H), 7.62 (d, J=8.4 Hz, 1H), 7.47-7.31 (m, 5H), 7.29 (d, J=2.4 Hz, 1H), 7.16 (d, J=6.8 Hz, 1H), 7.01 (d, J=2.4 Hz, 1H), 6.13-6.02 (m, 1H), 5.03 (q, J=6.8 Hz, 1H), 4.84-4.68 (m, 3H), 4.66-4.56 (m, 2H), 4.51 (t, J=8.0 Hz, 1H), 4.45-4.37 (m, 1H), 4.33-4.17 (m, 2H), 4.14-3.99 (m, 3H), 3.97-3.72 (m, 5H), 3.67-3.44 (m, 4H), 3.25-2.94 (m, 3H), 2.72 (s, 3H), 2.49-2.44 (m, 3H), 2.43-2.12 (m, 6H), 2.07-1.75 (m, 7H), 1.62-1.45 (m, 3H), 1.17-0.99 (m, 6H), 0.94-0.80 (m, 6H).

Exemplary Synthesis of [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 289)

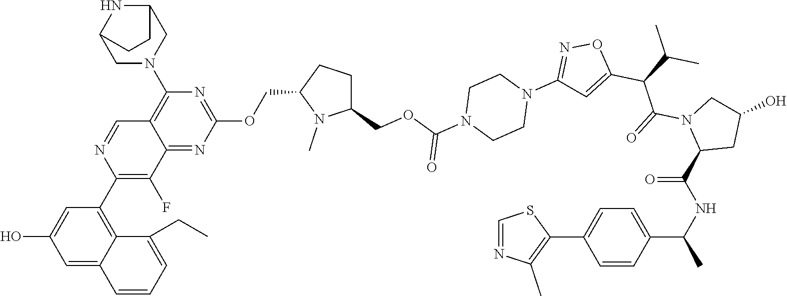 [SMILES: CCc1cccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-4-hydroxy-1-[(2S)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid salt, white solid). LC/MS (ESI) m/z: 1166.0 [M+H]⁺. ¹H NMR (400 MHz, DMSO-d₆) δ 9.09 (s, 1H), 9.01-8.89 (m, 1H), 8.24 (d, J=7.6 Hz, 1H), 7.66 (d, J=8.4 Hz, 1H), 7.53-7.22 (m, 6H), 7.11 (d, J=7.2 Hz, 1H), 6.97 (d, J=2.0 Hz, 1H), 6.22-6.14 (m, 1H), 4.86 (quin, J=7.2 Hz, 1H), 4.54-4.38 (m, 5H), 4.30-4.20 (m, 3H), 4.09-3.98 (m, 3H), 3.73-3.62 (m, 5H), 3.59-3.37 (m, 7H), 3.19-3.05 (m, 5H), 2.49-2.41 (m, 6H), 2.32-2.15 (m, 3H), 2.09-1.89 (m, 3H), 1.88-1.49 (m, 7H), 1.48-1.30 (m, 3H), 1.04-0.66 (m, 9H).

Exemplary Synthesis of [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl (2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate (Compound 280)

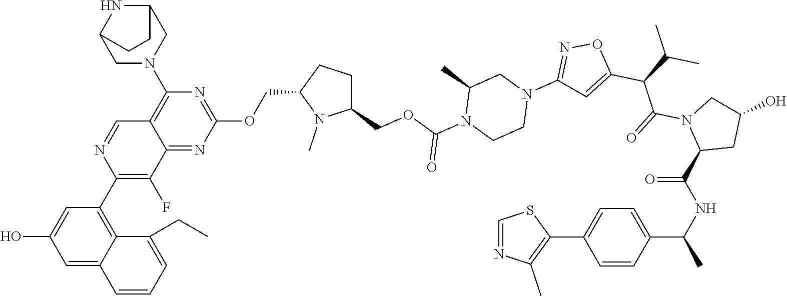 [SMILES: CCc1cccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)C[C@@H]6C)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-4-hydroxy-1-[(2S)-3-methyl-2-[3-[(3S)-3-methylpiperazin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1180.0 [M+H]⁺. ¹H NMR (400 MHz, DMSO-d₆) δ 9.09 (s, 1H), 9.01-8.90 (m, 1H), 8.24 (s, 1H), 7.66 (d, J=8.0 Hz, 1H), 7.53-7.19 (m, 6H), 7.11 (d, J=7.1 Hz, 1H), 6.97 (d, J=2.4 Hz, 1H), 6.18 (s, 1H), 5.04-4.81 (m, 1H), 4.51-4.37 (m, 4H), 4.33-4.12 (m, 4H), 4.03 (br d, J=3.6 Hz, 3H), 3.50-3.37 (m, 7H), 3.33-3.06 (m, 6H), 2.99-2.64 (m, 3H), 2.49-2.42 (m, 6H), 2.32-2.15 (m, 3H), 2.08-1.89 (m, 3H), 1.86-1.52 (m, 7H), 1.48-1.30 (m, 3H), 1.18-1.06 (m, 3H), 0.99-0.72 (m, 9H).

Exemplary Synthesis of [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl (2S)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazine-1-carboxylate (Compound 281)

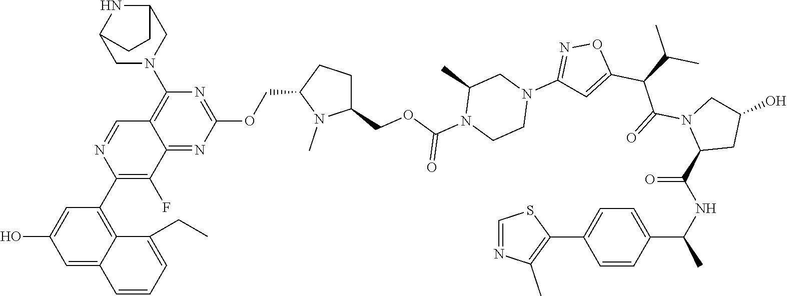 [SMILES: CCc1cccc2cc(O)cc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)C[C@@H]6C)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-[3-[(3S)-3-methylpiperazin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1180.0 [M+H]⁺. ¹H NMR (400 MHz, CD₃OD) δ 9.07 (s, 1H), 8.91-8.82 (m, 1H), 8.49 (d, J=9.6 Hz, 1H), 7.62 (d, J=8.0 Hz, 1H), 7.48-7.23 (m, 6H), 7.16 (d, J=7.2 Hz, 1H), 7.01 (d, J=2.4 Hz, 1H), 6.17-6.08 (m, 1H), 5.07-5.00 (m, 1H), 4.78-4.48 (m, 7H), 4.44-4.31 (m, 2H), 4.29-4.18 (m, 2H), 4.00-3.77 (m, 6H), 3.68-3.53 (m, 3H), 3.50-3.40 (m, 3H), 3.08-2.96 (m, 1H), 2.83 (d, J=11.6 Hz, 1H), 2.74-2.65 (m, 3H), 2.51-2.43 (m, 3H), 2.42-2.26 (m, 3H), 2.22-2.10 (m, 3H), 2.02 (s, 1H), 2.08-1.78 (m, 6H), 1.61-1.46 (m, 3H), 1.23 (d, J=6.4 Hz, 3H), 1.08-0.99 (m, 3H), 0.93-0.82 (m, 6H).

Exemplary Synthesis of [(2S,5S)-5-[[4-(azepan-1-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 272)

Step 1: Preparation of [(2S,5S)-5-[[4-(azepan-1-yl)-7-[8-ethyl-3-(methoxymethoxy)-1-naphthyl]-8-fluoro-pyrido[4,3-d]pyrimidin-2-yl]oxymethyl]-1-methyl-pyrrolidin-2-yl]methoxy-tert-butyl-diphenyl-silane

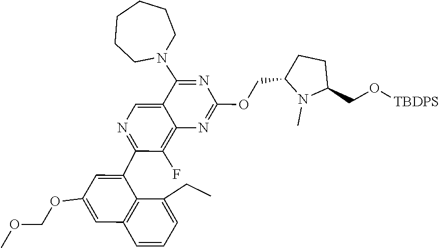

To a solution of 4-(azepan-1-yl)-7-[8-ethyl-3-(methoxymethoxy)-1-naphthyl]-8-fluoro-2-methylsulfonyl-pyrido[4,3-d]pyrimidine (600 mg, 1.11 mmol, 1.0 eq) and [(2S,5S)-5-[[tert-butyl(diphenyl)silyl]oxymethyl]-1-methyl-pyrrolidin-2-yl]methanol (427 mg, 1.11 mmol, 1.0 eq) in toluene (15 mL) was added 4 Å MS (600 mg), and the resulting mixture was stirred at 25° C. for 30 minutes. Sodium tert-butoxide (214 mg, 2.23 mmol, 2.0 eq) was then added at 0° C., and the reaction mixture was stirred at 0° C. for 1 hour. The reaction was quenched by addition of water/2N aqueous HCl (15 mL/0.7 mL) and extracted with ethyl acetate (30 mL×3). The combined organic extract was washed with brine (30 mL), dried over anhydrous Na₂SO₄, filtered, and concentrated under reduced pressure to afford [(2S,5S)-5-[[4-(azepan-1-yl)-7-[8-ethyl-3-(methoxymethoxy)-1-naphthyl]-8-fluoro-pyrido[4,3-d]pyrimidin-2-yl]oxymethyl]-1-methyl-pyrrolidin-2-yl]methoxy-tert-butyl-diphenyl-silane (952 mg, 0.93 mmol, 83% yield) as a yellow oil. LC/MS (ESI) m/z: 842.2 [M+H]⁺.

Step 2: Preparation of [(2S,5S)-5-[[4-(azepan-1-yl)-7-[8-ethyl-3-(methoxymethoxy)-1-naphthyl]-8-fluoro-pyrido[4,3-d]pyrimidin-2-yl]oxymethyl]-1-methyl-pyrrolidin-2-yl]methanol

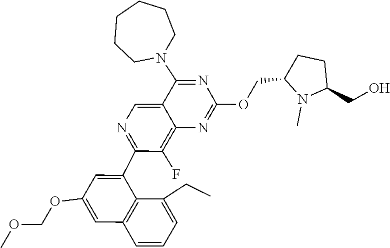 [SMILES: CCc1cccc2cc(OCOC)cc(-c3ncc4c(N5CCCCCC5)nc(OC[C@@H]5CC[C@@H](CO)N5C)nc4c3F)c12]

To a solution of [(2S,5S)-5-[[4-(azepan-1-yl)-7-[8-ethyl-3-(methoxymethoxy)-1-naphthyl]-8-fluoro-pyrido[4,3-d]pyrimidin-2-yl]oxymethyl]-1-methyl-pyrrolidin-2-yl]methoxy-tert-butyl-diphenyl-silane (952 mg, 1.13 mmol, 1.0 eq) in DMF (10 mL) was added cesium fluoride (1.4 g, 9.04 mmol, 8.0 eq), and the reaction mixture was stirred at 25° C. for 16 hours. The mixture was diluted with ethyl acetate (40 mL) and filtered. The filtrate was washed with water (10 mL×5), dried over anhydrous Na₂SO₄, filtered, and concentrated under reduced pressure. The resulting residue was purified by flash chromatography on SiO₂ (gradient: 0˜5% methanol in dichloromethane) to afford [(2S,5S)-5-[[4-(azepan-1-yl)-7-[8-ethyl-3-(methoxymethoxy)-1-naphthyl]-8-fluoro-pyrido[4,3-d]pyrimidin-2-yl]oxymethyl]-1-methyl-pyrrolidin-2-yl]methanol (464 mg, 0.74 mmol, 65% yield) as a white foam. LC/MS (ESI) m/z: 604.1 [M+H]⁺. ¹H NMR (400 MHz, CDCl₃) δ 9.14 (s, 1H), 7.71-7.69 (m, 1H), 7.54-7.53 (m, 1H), 7.43-7.40 (m, 1H), 7.25-7.23 (m, 1H), 7.20-7.19 (m, 1H), 5.32-5.31 (m, 2H), 4.75-4.73 (m, 2H), 4.05-3.96 (m, 6H), 3.52 (s, 3H), 2.93 (s, 2H), 2.05-2.04 (m, 6H), 1.64-1.63 (m, 12H), 0.96 (t, J=7.2 Hz, 3H).

Step 3: Preparation of [(2S,5S)-5-[[4-(azepan-1-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

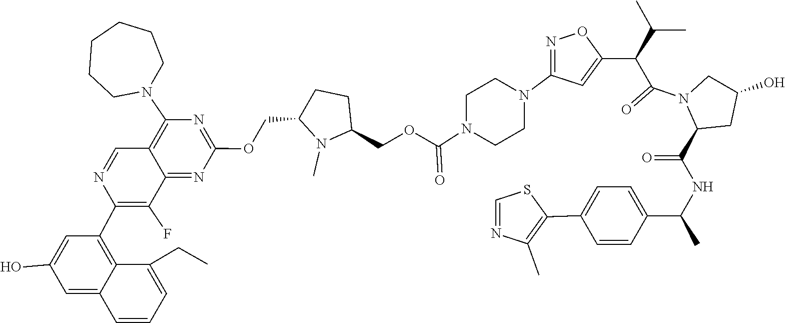 [SMILES: CCc1cccc2cc(O)cc(-c3ncc4c(N5CCCCCC5)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12]

The title compound was made in an analogous manner to [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from [(2S,5S)-5-[[4-(azepan-1-yl)-7-[8-ethyl-3-(methoxymethoxy)-1-naphthyl]-8-fluoro-pyrido[4,3-d]pyrimidin-2-yl]oxymethyl]-1-methyl-pyrrolidin-2-yl]methanol and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid salt, white solid). LC/MS (ESI) m/z: 1152.6 [M+H]⁺. ¹H NMR (400 MHz, CD₃OD) δ 9.14 (s, 1H), 8.87 (s, 1H), 7.62-7.60 (m, 1H), 7.42-7.35 (m, 5H), 7.29-7.28 (m, 1H), 7.16-7.14 (m, 1H), 7.04-7.03 (m, 1H), 6.12-6.11 (m, 1H), 5.04-5.00 (m, 1H), 4.64-4.59 (m, 2H), 4.53-4.48 (m, 1H), 4.40-4.40 (m, 1H), 4.33-4.20 (m, 2H), 4.11-4.08 (m, 4H), 3.84-3.79 (m, 1H), 3.65-3.49 (m, 8H), 3.21 (s, 4H), 2.75-2.74 (m, 3H), 2.47 (s, 3H), 2.38-2.14 (m, 6H), 2.03-1.81 (m, 7H), 1.69 (s, 4H) 1.50-1.48 (m, 3H), 1.05-1.03 (m, 3H), 0.91-0.87 (m, 6H).

Exemplary Synthesis of [(2S,5S)-5-[[4-(azepan-1-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl (3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carboxylate (Compound 230)

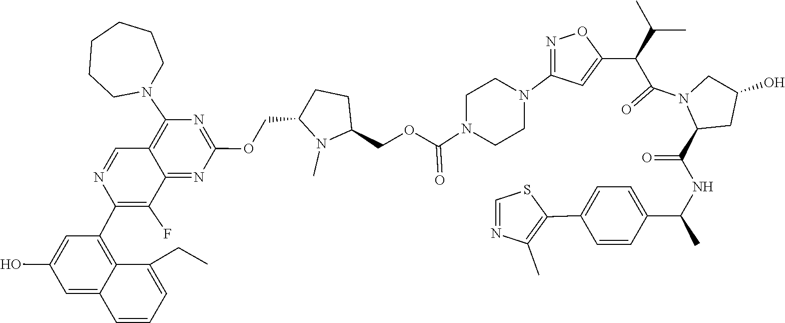 [SMILES: CCc1cccc2cc(O)cc(-c3ncc4c(N5CCCCCC5)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12]

The title compound was made in an analogous manner to [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from [(2S,5S)-5-[[4-(azepan-1-yl)-7-[8-ethyl-3-(methoxymethoxy)-1-naphthyl]-8-fluoro-pyrido[4,3-d]pyrimidin-2-yl]oxymethyl]-1-methyl-pyrrolidin-2-yl]methanol and (2S,4R)-4-hydroxy-1-[(2R)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide. (white solid). LC/MS (ESI) m/z: 1167.0 [M+H]⁺. ¹H NMR (400 MHz, CD₃OD) δ 9.23-9.06 (m, 1H), 8.89-8.82 (m, 1H), 7.63 (d, J=8.0 Hz, 1H), 7.47-7.22 (m, 6H), 7.16 (d, J=7.2 Hz, 1H), 7.08-6.99 (m, 1H), 6.12-6.00 (m, 1H), 5.08-4.96 (m, 1H), 4.62-4.38 (m, 5H), 4.28-4.04 (m, 7H), 3.96-3.70 (m, 3H), 3.65-3.34 (m, 5H), 3.18-3.04 (m, 2H), 2.63 (s, 3H), 2.52-2.42 (m, 3H), 2.40-2.23 (m, 3H), 2.20-1.97 (m, 7H), 1.96-1.79 (m, 2H), 1.79-1.63 (m, 5H), 1.60-1.44 (m, 3H), 1.15-0.99 (m, 6H), 0.94-0.81 (m, 6H).

Exemplary Synthesis of [(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carboxylate (Compound 229)

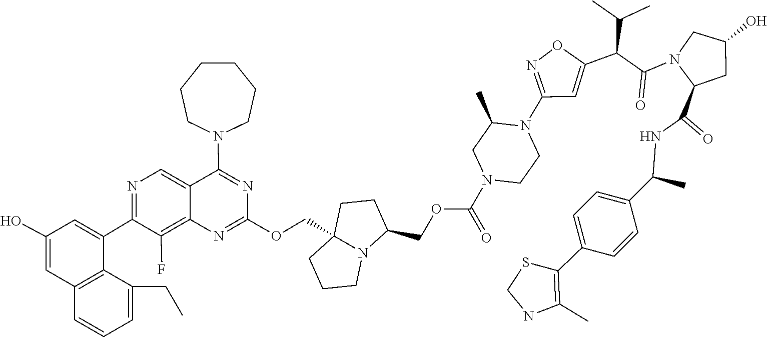 [SMILES: CCc1cccc2cc(O)cc(-c3ncc4c(N5CCCCCC5)nc(OC[C@@]56CCCN5[C@H](COC(=O)N5CCN(c7cc([C@H](C(=O)N8C[C@H](O)C[C@H]8C(=O)N[C@@H](C)c8ccc(C9=C(C)NCS9)cc8)C(C)C)on7)[C@H](C)C5)CC6)nc4c3F)c12]

The title compound was prepared in an analogous manner to [(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from [(3S,8S)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-4-hydroxy-1-[(2R)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide. (formic acid, white solid). LC/MS (ESI) m/z: 1193.0 [M+H]⁺. ¹H NMR (400 MHz, CD₃OD) δ 9.14 (s, 1H), 8.87 (s, 1H), 8.53 (s, 1H), 7.62 (d, J=8.0 Hz, 1H), 7.46-7.32 (m, 5H), 7.28 (d, J=2.4 Hz, 1H), 7.17 (s, 1H), 7.02 (d, J=2.8 Hz, 1H), 6.14-6.03 (m, 1H), 5.24-5.15 (m, 1H), 5.03 (d, J=7.2 Hz, 1H), 4.50 (t, J=8.4 Hz, 2H), 4.43 (s, 4H), 4.11 (t, J=5.2 Hz, 5H), 3.96-3.70 (m, 4H), 3.63 (d, J=10.0 Hz, 2H), 3.06-2.97 (m, 1H), 3.15 (dd, J=9.6, 11.6 Hz, 2H), 2.51-2.41 (m, 3H), 2.40-2.25 (m, 4H), 2.22-2.11 (m, 2H), 1.92-1.91 (m, 1H), 1.91-1.89 (m, 1H), 2.10-1.87 (m, 9H), 1.71 (s, 4H), 1.59-1.49 (m, 3H), 1.34-1.26 (m, 1H), 1.24-1.23 (m, 1H), 1.11 (d, J=6.4 Hz, 3H), 1.04 (d, J=6.8 Hz, 3H), 0.93-0.84 (m, 6H).

Exemplary Synthesis of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl (3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carboxylate (Compound 227)

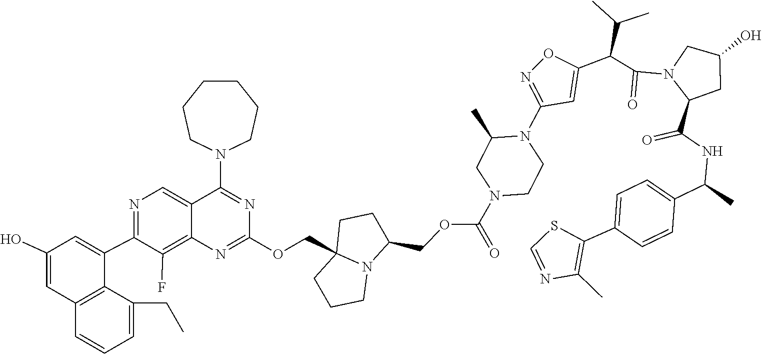 [SMILES: CCc1cccc2cc(O)cc(-c3ncc4c(N5CCCCCC5)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)[C@H](C)C5)CC6)nc4c3F)c12]

The title compound was prepared in an analogous manner to [(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-4-hydroxy-1-[(2R)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1192.6 [M+H]⁺. ¹H NMR (400 MHz, CD₃OD) δ 9.17 (s, 1H), 8.87 (s, 1H), 7.63-7.61 (m, 1H), 7.44-7.30 (m, 6H), 7.16-7.14 (m, 1H), 7.06-7.04 (m, 1H), 6.06-6.04 (m, 1H), 5.06-5.00 (m, 2H), 4.54-4.37 (m, 5H), 4.17-4.03 (m, 7H), 3.90-3.78 (m, 3H), 3.66-3.48 (m, 3H), 3.15-3.08 (m, 3H), 2.47 (s, 3H), 2.38-2.26 (m, 4H), 2.16-1.85 (m, 14H), 1.69 (s, 4H) 1.50-1.49 (m, 3H), 1.06-1.04 (m, 6H), 0.91-0.87 (m, 6H).

Exemplary Synthesis of tert-butyl 3-[7-(8-ethyl-1-naphthyl)-8-fluoro-2-[[(3S,8R)-3-[[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carbonyl]oxymethyl]-1,2,3,5,6,7-hexahydropyrrolizin-8-yl]methoxy]pyrido[4,3-d]pyrimidin-4-yl]-3,8-diazabicyclo[3.2.1]octane-8-carboxylate (Compound 226)

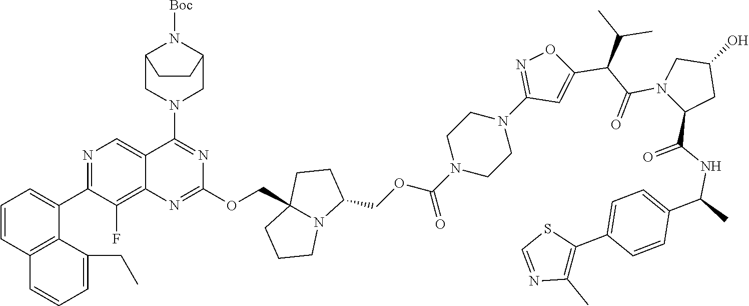 [SMILES: CCc1cccc2cccc(-c3ncc4c(N5CC6CCC(C5)N6C(=O)OC(C)(C)C)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to [(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-(8-ethyl-1-naphthyl)-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1190.0 [M+H]⁺. ¹H NMR (400 MHz, CD₃OD) δ 9.10 (s, 1H), 8.88 (s, 1H), 8.05 (d, J=8.4 Hz, 1H), 7.87 (d, J=8.0 Hz, 1H), 7.59-7.47 (m, 2H), 7.4-7.37 (m, 6H), 6.06 (s, 1H), 5.05-5.00 (m, 1H), 4.78-4.69 (m, 2H), 4.60 (s, 3H), 4.52-4.48 (m, 1H), 4.44-4.34 (m, 3H), 4.28-4.17 (m, 1H), 4.15-4.05 (m, 2H), 3.94-3.88 (m, 3H), 3.84-3.73 (m, 3H), 3.67-3.56 (m, 2H), 3.23-2.96 (m, 5H), 2.49-2.28 (m, 7H), 2.14-2.08 (m, 2H), 2.04-1.78 (m, 11H), 1.57-1.46 (m, 3H), 1.13-1.02 (m, 6H), 0.95-0.86 (m, 6H).

Exemplary Synthesis of [(3S,8R)-8-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 225)

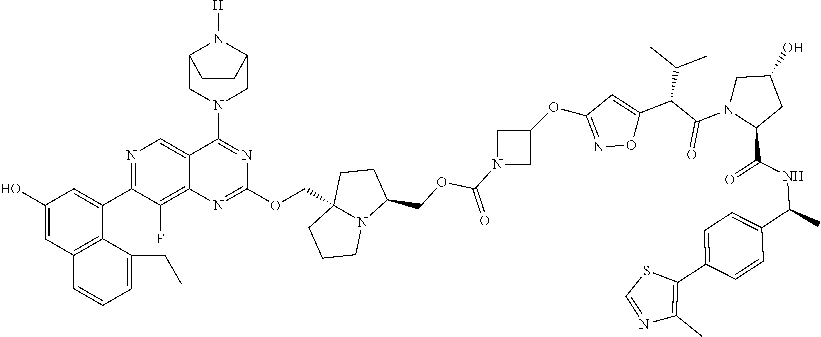 [SMILES: CCc1cccc2cc(O)cc(-c3ncc4c(N5CC6CCC(C5)N6)nc(OC[C@@]56CCCN5[C@H](COC(=O)N5CC(Oc7cc([C@@H](C(=O)N8C[C@H](O)C[C@H]8C(=O)N[C@@H](C)c8ccc(-c9scnc9C)cc8)C(C)C)on7)C5)CC6)nc4c3F)c12]

The title compound was prepared in an analogous manner to [(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-(8-ethyl-1-naphthyl)-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1162.9 [M+H]⁺. ¹H NMR (400 MHz, CD₃OD) δ 9.11 (d, J=3.2 Hz, 1H), 8.90-8.83 (m, 1H), 8.05 (dd, J=4.8, 7.6 Hz, 1H), 7.87 (br d, J=7.6 Hz, 1H), 7.63-7.27 (m, 8H), 6.06-5.94 (m, 1H), 5.15-5.07 (m, 1H), 5.02 (q, J=6.8 Hz, 1H), 4.58 (br s, 5H), 4.44-4.31 (m, 4H), 4.26-4.20 (m, 1H), 4.13-4.06 (m, 1H), 3.97 (br s, 3H), 3.89-3.72 (m, 3H), 3.69 (dd, J=4.0, 10.0 Hz, 1H), 3.61 (br d, J=11.2 Hz, 1H), 3.30-3.21 (m, 2H), 3.13-3.03 (m, 1H), 2.50-2.45 (m, 3H), 2.44-2.28 (m, 4H), 2.21-1.85 (m, 13H), 1.57-1.47 (m, 3H), 1.09-1.02 (m, 3H), 0.96-0.84 (m, 6H).

Exemplary Synthesis of [(2S,5S)-5-[[4-(azepan-1-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (Compound 241)

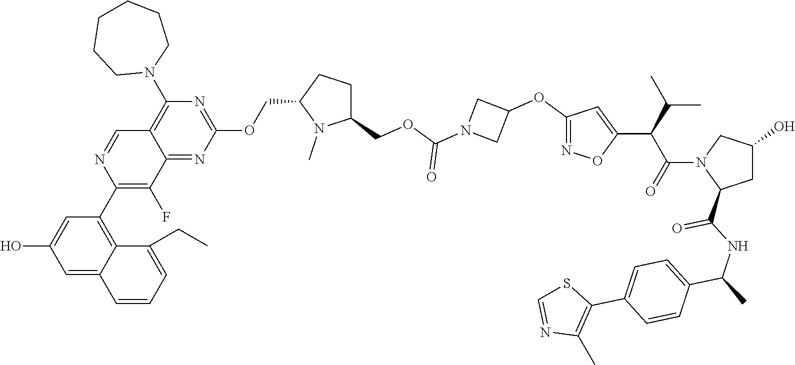 [SMILES: CCc1cccc2cc(O)cc(-c3ncc4c(N5CCCCCC5)nc(OC[C@@H]5CC[C@@H](COC(=O)N6CC(Oc7cc([C@H](C(=O)N8C[C@H](O)C[C@H]8C(=O)N[C@@H](C)c8ccc(-c9scnc9C)cc8)C(C)C)on7)C6)N5C)nc4c3F)c12]

The title compound was made in an analogous manner to [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from [(2S,5S)-5-[[4-(azepan-1-yl)-7-[8-ethyl-3-(methoxymethoxy)-1-naphthyl]-8-fluoro-pyrido[4,3-d]pyrimidin-2-yl]oxymethyl]-1-methyl-pyrrolidin-2-yl]methanol and (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). LC/MS (ESI) m/z: 1139.9. [M+H]⁺. ¹H NMR (400 MHz, CD₃OD) δ 9.15-9.11 (m, 1H), 8.67 (s, 1H), 7.62 (d, J=8.0 Hz, 1H), 7.43-7.28 (m, 6H), 7.16 (d, J=6.4 Hz, 1H), 7.04-7.02 (m, 1H), 6.04-5.96 (m, 1H), 5.16-5.12 (m, 1H), 5.01 (q, J=7.2 Hz, 1H), 4.50-4.49 (m, 1H), 4.41-4.33 (m, 3H), 4.21-4.09 (m, 6H), 4.01-3.98 (m, 2H), 3.82-3.74 (m, 1H), 3.71-3.65 (m, 1H), 3.61-3.54 (m, 1H), 3.50-3.36 (m, 2H), 2.60 (s, 3H), 2.48-2.46 (m, 3H), 2.40-2.26 (m, 3H), 2.18-2.05 (m, 7H), 1.95-1.80 (m, 2H), 1.75-1.72 (m, 5H), 1.56-1.48 (m, 3H), 1.34-1.28 (m, 2H), 1.03 (d, J=6.4 Hz, 1H), 0.92-0.86 (m, 6H).

Exemplary Synthesis of ((2S,5S)-5-(((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)methyl)-1-methylpyrrolidin-2-yl)methyl 4-((5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)piperidine-1-carboxylate (Compound 255)

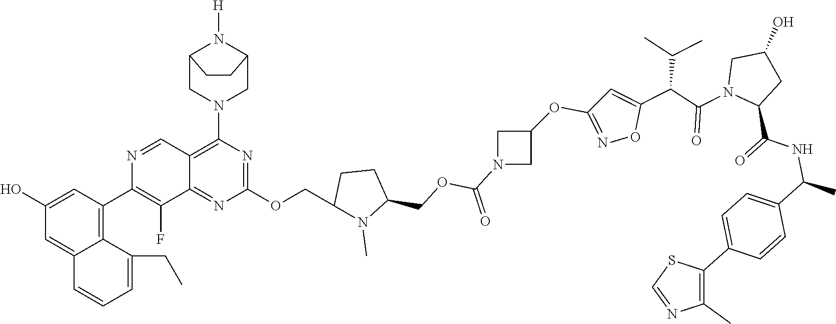 [SMILES: CCc1cccc2cc(O)cc(-c3ncc4c(N5CC6CCC(C5)N6)nc(OCC5CC[C@@H](COC(=O)N6CC(Oc7cc([C@@H](C(=O)N8C[C@H](O)C[C@H]8C(=O)N[C@@H](C)c8ccc(-c9scnc9C)cc8)C(C)C)on7)C6)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-4-hydroxy-1-((S)-3-methyl-2-(3-(piperidin-4-yloxy)isoxazol-5-yl)butanoyl)-N—((S)-1-(4-(4-methylthiazol-5-yl)phenyl)ethyl)pyrrolidine-2-carboxamide. (formic acid slat, white solid). LC/MS (ESI) m/z: 591.1 [M/2+H]⁺. ¹H NMR (400 MHz, CD₃OD) δ 9.05 (s, 1H), 8.90-8.81 (m, 1H), 8.49 (s, 1H), 7.63 (d, J=8.4 Hz, 1H), 7.50-7.38 (m, 3H), 7.38-7.32 (m, 3H), 7.29 (d, J=2.8 Hz, 1H), 7.15 (br d, J=7.2 Hz, 1H), 7.04-6.97 (m, 1H), 6.07-5.95 (m, 1H), 4.75-4.67 (m, 3H), 4.66-4.47 (m, 6H), 4.45-4.31 (m, 1H), 4.29-4.11 (m, 2H), 3.94-3.86 (m, 2H), 3.86-3.73 (m, 4H), 3.72-3.64 (m, 3H), 3.51-3.45 (m, 1H), 3.40 (br dd, J=2.0, 4.4 Hz, 2H), 2.71-2.63 (m, 3H), 2.50-2.42 (m, 3H), 2.41-2.24 (m, 4H), 2.22-2.10 (m, 3H), 2.01-1.88 (m, 7H), 1.82-1.75 (m, 1H), 1.73-1.62 (m, 2H), 1.47 (d, J=7.2 Hz, 3H), 1.39-1.24 (m, 1H), 1.05 (d, J=6.4 Hz, 3H), 0.95-0.85 (m, 6H).

Exemplary Synthesis of ((2S,5S)-5-(((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)methyl)-1-methylpyrrolidin-2-yl)methyl 4-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)piperidine-1-carboxylate (Compound 252)

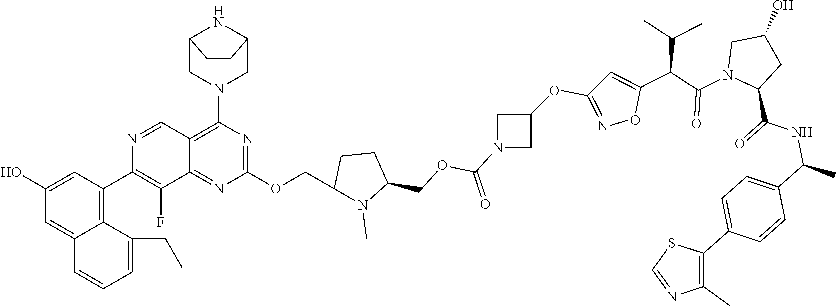 [SMILES: CCc1cccc2cc(O)cc(-c3ncc4c(N5CC6CCC(C5)N6)nc(OCC5CC[C@@H](COC(=O)N6CC(Oc7cc([C@H](C(=O)N8C[C@H](O)C[C@H]8C(=O)N[C@@H](C)c8ccc(-c9scnc9C)cc8)C(C)C)on7)C6)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-4-hydroxy-1-((R)-3-methyl-2-(3-(piperidin-4-yloxy)isoxazol-5-yl) butanoyl)-N—((S)-1-(4-(4-methylthiazol-5-yl)phenyl)ethyl)pyrrolidine-2-carboxamide. (formic acid salt, white solid). LC/MS (ESI) m/z: 591.1 [M/2+H]⁺. ¹H NMR (400 MHz, CD₃OD) δ 9.12-9.05 (m, 1H), 8.91-8.84 (m, 1H), 8.44-8.34 (m, 2H), 7.64 (d, J=8.0 Hz, 1H), 7.48-7.38 (m, 4H), 7.38-7.33 (m, 1H), 7.30 (d, J=2.4 Hz, 1H), 7.20-7.14 (m, 1H), 7.04-6.99 (m, 1H), 6.00 (d, J=2.0 Hz, 1H), 5.07-4.99 (m, 1H), 4.80-4.69 (m, 5H), 4.66-4.58 (m, 3H), 4.50 (br t, J=8.4 Hz, 1H), 4.46-4.39 (m, 1H), 4.32-4.22 (m, 2H), 4.12-4.03 (m, 2H), 3.93-3.87 (m, 1H), 3.86-3.80 (m, 2H), 3.79-3.72 (m, 2H), 3.68 (d, J=9.9 Hz, 1H), 3.64-3.57 (m, 2H), 3.55-3.47 (m, 1H), 3.42-3.34 (m, 2H), 2.78-2.72 (m, 3H), 2.51-2.44 (m, 3H), 2.41-2.32 (m, 2H), 2.32-2.24 (m, 1H), 2.23-2.14 (m, 3H), 2.07-2.01 (m, 4H), 1.99-1.90 (m, 3H), 1.87-1.78 (m, 1H), 1.77-1.68 (m, 2H), 1.56-1.49 (m, 3H), 1.05 (d, J=6.5 Hz, 3H), 0.94-0.84 (m, 6H).

Exemplary Synthesis of ((2S,5S)-5-(((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)methyl)-1-methylpyrrolidin-2-yl)methyl 6-(5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)-2,6-diazaspiro[3.3]heptane-2-carboxylate (Compound 242)

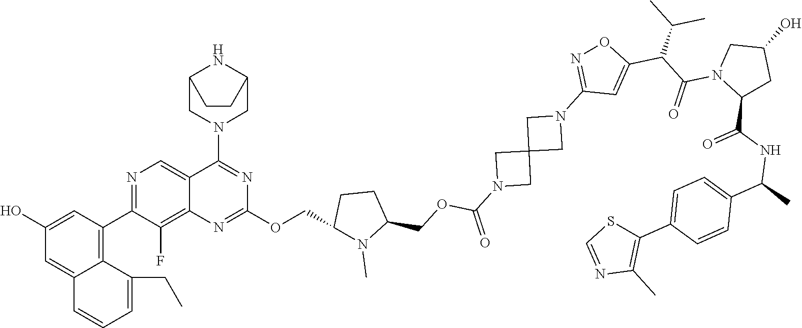

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-1-((S)-2-(3-(2,6-diazaspiro[3.3]heptan-2-yl)isoxazol-5-yl)-3-methyl butanoyl)-4-hydroxy-N—((S)-1-(4-(4-methylthiazol-5-yl)phenyl)ethyl)pyrrolidine-2-carboxamide. (formic acid salt, white solid). LC/MS (ESI) m/z: 589.7 [M+H]⁺. ¹H NMR (400 MHz, CD₃OD) δ 9.06 (d, J=2.0 Hz, 1H), 8.90-8.83 (m, 1H), 8.48-8.39 (m, 1H), 7.63 (d, J=8.0 Hz, 1H), 7.50-7.43 (m, 1H), 7.43-7.32 (m, 5H), 7.29 (t, J=2.4 Hz, 1H), 7.16 (br d, J=7.2 Hz, 1H), 7.01 (d, J=2.4 Hz, 1H), 5.92-5.81 (m, 1H), 4.80-4.64 (m, 3H), 4.63-4.53 (m, 4H), 4.45-4.34 (m, 1H), 4.26-4.19 (m, 1H), 4.16-4.07 (m, 5H), 4.05-3.92 (m, 6H), 3.86-3.75 (m, 2H), 3.73-3.61 (m, 3H), 3.54-3.46 (m, 1H), 3.42 (br d, J=5.6 Hz, 1H), 2.73-2.63 (m, 3H), 2.50-2.44 (m, 3H), 2.41-2.31 (m, 2H), 2.30-2.23 (m, 1H), 2.22-2.13 (m, 3H), 2.03-1.92 (m, 5H), 1.89-1.71 (m, 2H), 1.60-1.44 (m, 3H), 1.37-1.26 (m, 1H), 1.04 (d, J=6.6 Hz, 3H), 0.94-0.86 (m, 6H).

Exemplary Synthesis of ((2S,5S)-5-(((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)methyl)-1-methylpyrrolidin-2-yl)methyl 6-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)-2,6-diazaspiro[3.3]heptane-2-carboxylate (Compound 256)

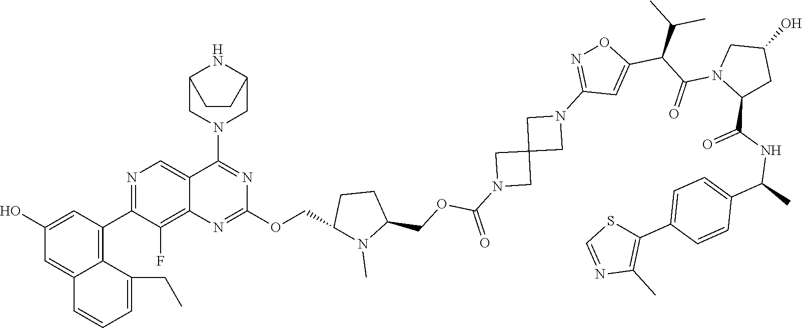 [SMILES: CCc1cccc2cc(O)cc(-c3ncc4c(N5CC6CCC(C5)N6)nc(OC[C@@H]5CC[C@@H](COC(=O)N6CC7(C6)CN(c6cc([C@H](C(=O)N8C[C@H](O)C[C@H]8C(=O)N[C@@H](C)c8ccc(-c9scnc9C)cc8)C(C)C)on6)C7)N5C)nc4c3F)c12]

The title compound was made in an analogous manner to [(2S,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate and (2S,4R)-1-((R)-2-(3-(2,6-diazaspiro[3.3]heptan-2-yl)isoxazol-5-yl)-3-methyl butanoyl)-4-hydroxy-N—((S)-1-(4-(4-methylthiazol-5-yl)phenyl)ethyl)pyrrolidine-2-carboxamide. (formic acid salt, white solid). LC/MS (ESI) m/z: 589.8 [M+H]⁺. ¹H NMR (400 MHz, CD₃OD) & 9.07 (s, 1H), 8.91-8.82 (m, 1H), 8.49-8.38 (m, 1H), 7.63 (d, J=8.0 Hz, 1H), 7.48-7.39 (m, 4H), 7.37 (br t, J=7.6 Hz, 2H), 7.32-7.27 (m, 1H), 7.17 (br d, J=6.8 Hz, 1H), 7.02 (t, J=3.2 Hz, 1H), 5.89-5.79 (m, 1H), 5.11-4.95 (m, 2H), 4.82-4.67 (m, 3H), 4.63-4.55 (m, 2H), 4.54-4.46 (m, 1H), 4.45-4.37 (m, 1H), 4.28-4.20 (m, 1H), 4.15 (br s, 5H), 4.07-3.95 (m, 6H), 3.91-3.78 (m, 3H), 3.67-3.56 (m, 2H), 3.53-3.40 (m, 2H), 2.79-2.65 (m, 3H), 2.52-2.44 (m, 3H), 2.39-2.26 (m, 3H), 2.23-2.12 (m, 3H), 2.09-1.94 (m, 5H), 1.90-1.75 (m, 2H), 1.60-1.49 (m, 3H), 1.04 (br d, J=6.4 Hz, 3H), 0.93-0.82 (m, 6H).

Exemplary Synthesis of [(3R,8S)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 334)

Step 1: Preparation of tert-butyl 3-[2-[[(3R,8S)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate

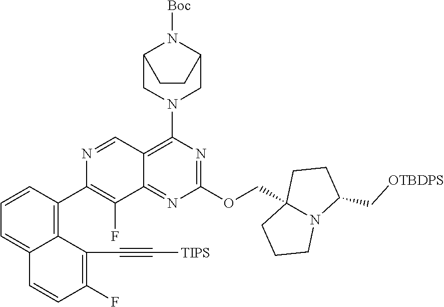

To a mixture of tert-butyl 3-[8-fluoro-7-[7-fluoro-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate (500 mg, 0.66 mmol, 1 eq), [(3R,8S)-3-[[tert-butyl(diphenyl)silyl]oxymethyl]-1,2,3,5,6,7-hexahydropyrrolizin-8-yl]methanol (269 mg, 0.66 mmol, 1 eq), and 4 Å MS (500 mg, 0.66 mmol, 1.00 eq) in toluene (10 mL) at 0° C. was added sodium tert-butoxide (189 mg, 1.97 mmol, 3 eq), and the reaction mixture was stirred at 0° C. for 30 minutes. The pH was adjusted to pH ˜7 by progressively adding hydrochloric acid (2 M). The mixture was diluted with water (20 mL) and extracted with ethyl acetate (20 mL×3). The combined organic extracts were washed brine (10 mL), dried over anhydrous sodium sulfate, filtered, and concentrated under reduced pressure. The resulting residue was purified by flash silica gel chromatography (gradient: 0˜40% Ethyl acetate/Petroleum ether) to afford tert-butyl 3-[2-[[(3R,8S)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate (390 mg, 0.35 mol, 54% yield) as a yellow solid. LC/MS (ESI) m/z: 546.7 [M/2+H]⁺.

Step 2: Preparation of tert-butyl 3-[7-(8-ethynyl-7-fluoro-1-naphthyl)-8-fluoro-2-[[(3R,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

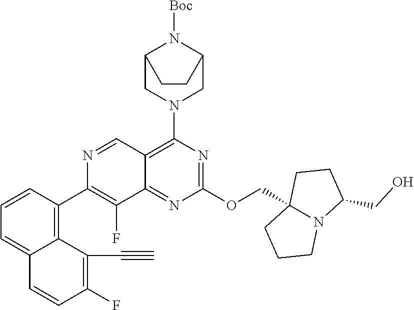 [SMILES: C#Cc1c(F)ccc2cccc(-c3ncc4c(N5CC6CCC(C5)N6C(=O)OC(C)(C)C)nc(OC[C@@]56CCCN5[C@@H](CO)CC6)nc4c3F)c12]

To a solution of tert-butyl 3-[2-[[(3R,8S)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate (390 mg, 0.36 mmol, 1 eq) in DMF (3 mL) was added cesium fluoride (1.09 g, 7.15 mmol, 20 eq), and the reaction mixture was stirred at 25° C. for 16 h. The mixture was diluted with water 30 mL and extracted with ethyl acetate (20 mL×2). The combined organic extracts were washed with brine (20 mL×2), dried over anhydrous sodium sulfate, filtered, and concentrated. The residue was purified by flash silica gel chromatography (gradient: 0˜40% Ethyl acetate/Petroleum ether gradient) to afford tert-butyl 3-[7-(8-ethynyl-7-fluoro-1-naphthyl)-8-fluoro-2-[[(3R,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (200 mg, 0.28 mmol, 80% yield) as a white foam. LC/MS (ESI) m/z: 697.5 [M+H]⁺.

Step 3: Preparation of [(3R,8S)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

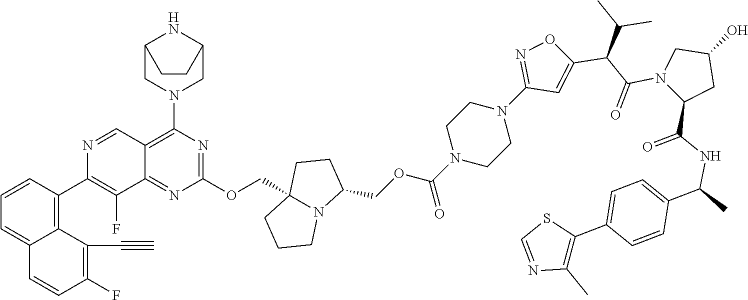 [SMILES: C#Cc1c(F)ccc2cccc(-c3ncc4c(N5CC6CCC(C5)N6)nc(OC[C@@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was made in an analogous manner to [(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-(8-ethynyl-7-fluoro-1-naphthyl)-8-fluoro-2-[[(3R,8S)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid salt, green solid). LC/MS (ESI) m/z: 1189.3 [M+H]⁺. ¹H NMR (400 MHz, CD₃OD) δ 9.08 (s, 1H), 8.89-8.84 (m, 1H), 8.47 (s, 1H), 8.14-8.07 (m, 2H), 7.68-7.63 (m, 2H), 7.52 (t, J=7.6 Hz, 1H), 7.47-7.37 (m, 5H), 6.10 (d, J=8.8 Hz, 1H), 5.04 (q, J=7.2 Hz, 1H), 4.80-4.70 (m, 2H), 4.56-4.51 (m, 1H), 4.47-4.38 (m, 3H), 4.25 (dt, J=4.0, 11.2 Hz, 1H), 4.12 (dd, J=6.4, 10.8 Hz, 1H), 3.96 (s, 2H), 3.89-3.80 (m, 3H), 3.76-3.69 (m, 1H), 3.65-3.42 (m, 8H), 3.22-3.06 (m, 5H), 2.90 (s, 1H), 2.51-2.44 (m, 3H), 2.43-2.30 (m, 2H), 2.22-2.09 (m, 3H), 2.03-1.88 (m, 10H), 1.57 (dd, J=4.8, 6.8 Hz, 1H), 1.52 (d, J=7.2 Hz, 2H), 1.05 (d, J=6.8 Hz, 3H), 0.93-0.84 (m, 3H).

Exemplary Synthesis of [(3R,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 333)

Step 1: Preparation of tert-butyl 3-[2-[[(3R,8R)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate

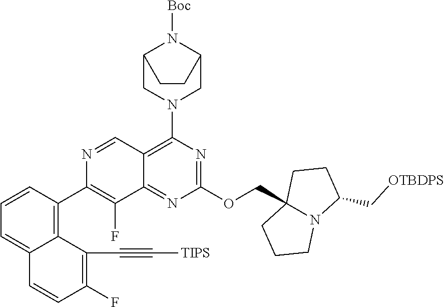

To a mixture of tert-butyl 3-[8-fluoro-7-[7-fluoro-8-(2-triisopropylsilylethynyl)-1-naphthyl]-2-methylsulfonyl-pyrido[4,3-d]pyrimidin-4-yl]-3,8-diazabicyclo[3.2.1]octane-8-carboxylate (500 mg, 0.66 mmol, 1 eq), [(3R,8R)-3-[[tert-butyl(diphenyl)silyl]oxymethyl]-1,2,3,5,6,7-hexahydropyrrolizin-8-yl]methanol (269 mg, 0.66 mmol, 1 eq), and 4 Å MS (500 mg) in toluene (10 mL) at 0° C. was added sodium tert-butoxide (189 mg, 1.97 mmol, 3 eq), and the reaction mixture was stirred at 0° C. for 30 minutes. The pH was adjusted to pH ˜7 by progressively adding hydrochloric acid (2 M). The mixture was diluted with water (20 mL) and extracted with ethyl acetate (20 mL×3). The combined organic extracts were washed brine (10 mL), dried over anhydrous sodium sulfate, filtered, and concentrated. The residue was purified by flash silica gel chromatography (gradient: 0˜40% Ethyl acetate/Petroleum ether) to afford tert-butyl 3-[2-[[(3R,8R)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate (400 mg, 0.35 mol, 54% yield) as a yellow solid. LC/MS (ESI) m/z: 546.8 [M/2+H]⁺.

Step 2: Preparation of tert-butyl 3-[7-(8-ethynyl-7-fluoro-1-naphthyl)-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

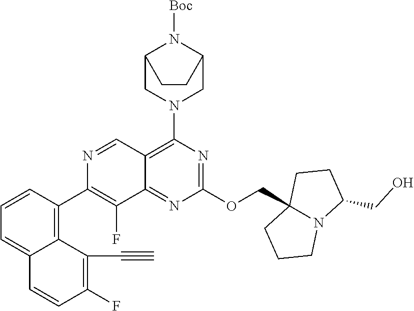 [SMILES: C#Cc1c(F)ccc2cccc(-c3ncc4c(N5CC6CCC(C5)N6C(=O)OC(C)(C)C)nc(OC[C@]56CCCN5[C@@H](CO)CC6)nc4c3F)c12]

To a solution of tert-butyl 3-[2-[[(3R,8R)-3-[[tert-butyl(diphenyl)silyl]oxymethyl]-1,2,3,5,6,7-hexahydropyrrolizin-8-yl]methoxy]-8-fluoro-7-[7-fluoro-8-(2-triisopropylsilylethynyl)-1-naphthyl]pyrido[4,3-d]pyrimidin-4-yl]-3,8-diazabicyclo[3.2.1]octane-8-carboxylate (400 mg, 0.37 mmol, 1 eq) in DMF (3 mL) was added cesium fluoride (1.11 g, 7.33 mmol, 0.27 mL, 20 eq), and the reaction mixture was stirred at 25° C. for 16 h. The mixture was diluted with water 30 mL and extracted with ethyl acetate (20 mL×2). The combined organic extracts were washed with brine (20 mL×2), dried over anhydrous sodium sulfate, filtered, and concentrated. The residue was purified by flash silica gel chromatography (gradient: 0˜40% Ethyl acetate/Petroleum ether) to afford tert-butyl 3-[7-(8-ethynyl-7-fluoro-1-naphthyl)-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (160 mg, 0.23 mmol, 63% yield) as a white foam. LC/MS (ESI) m/z: 697.5 [M+H]⁺.

Step 3: Preparation of [(3R,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

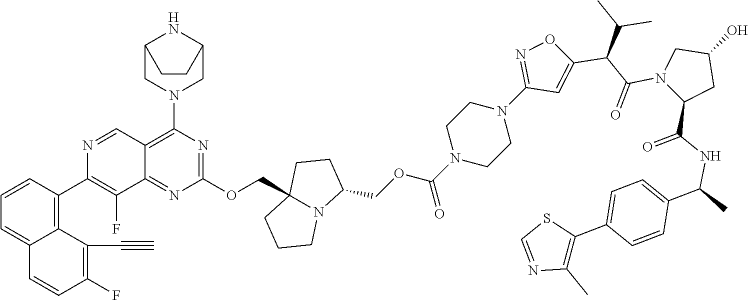 [SMILES: C#Cc1c(F)ccc2cccc(-c3ncc4c(N5CC6CCC(C5)N6)nc(OC[C@]56CCCN5[C@@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was made in an analogous manner to [(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-(8-ethynyl-7-fluoro-1-naphthyl)-8-fluoro-2-[[(3R,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid salt, green solid). LC/MS (ESI) m/z: 1189.4 [M+H]⁺. ¹H NMR (400 MHz, CD₃OD) δ 9.10 (d, J=2.0 Hz, 1H), 8.89-8.86 (m, 1H), 8.45 (s, 1H), 8.16-8.08 (m, 2H), 7.69-7.61 (m, 2H), 7.48-7.38 (m, 5H), 7.38-7.34 (m, 1H), 6.15-6.06 (m, 1H), 5.07-5.00 (m, 1H), 4.78-4.65 (m, 4H), 4.62-4.54 (m, 2H), 4.51 (t, J=8.0 Hz, 2H), 4.46-4.37 (m, 2H), 4.23-4.09 (m, 1H), 3.93-3.78 (m, 5H), 3.67-3.48 (m, 7H), 3.43 (d, J=2.4 Hz, 1H), 3.22 (s, 2H), 2.49-2.45 (m, 3H), 2.43-2.29 (m, 3H), 2.25-2.15 (m, 3H), 2.13-1.87 (m, 10H), 1.57 (dd, J=2.0, 7.2 Hz, 1H), 1.52 (d, J=7.2 Hz, 2H), 1.33-1.27 (m, 1H), 1.05 (d, J=6.4 Hz, 3H), 0.91-0.85 (m, 3H).

Exemplary Synthesis of [(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 278)

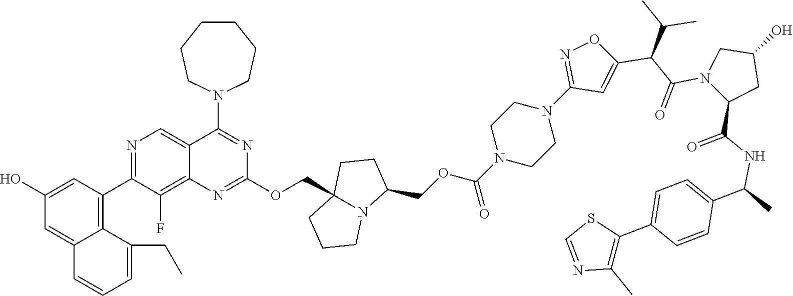 [SMILES: CCc1cccc2cc(O)cc(-c3ncc4c(N5CCCCCC5)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was prepared in an analogous manner to [(3S,8S)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from [(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methanol and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid salt, white solid). LC/MS (ESI) m/z: 1179.8 [M+H]⁺. ¹H NMR (400 MHz, DMSO-d₆) δ 10.17-10.02 (m, 1H), 9.96 (d, J=3.6 Hz, 1H), 9.22 (s, 1H), 8.98 (s, 1H), 8.42 (dd, J=8.2, 11.6 Hz, 1H), 8.13 (s, 1H), 7.66 (d, J=8.4 Hz, 1H), 7.45-7.32 (m, 5H), 7.29 (s, 1H), 7.12 (d, J=7.2 Hz, 1H), 6.99 (s, 1H), 6.19-6.01 (m, 1H), 5.13 (s, 1H), 4.91 (t, J=7.2 Hz, 1H), 4.66-4.49 (m, 2H), 4.45-4.24 (m, 4H), 4.09-3.99 (m, 4H), 3.84 (dd, J=4.4, 6.0 Hz, 2H), 3.70 (dd, J=3.6, 10.4 Hz, 2H), 3.61-3.55 (m, 2H), 3.46 (s, 4H), 3.32-3.25 (m, 2H), 3.22-3.08 (m, 5H), 2.45 (d, J=1.6 Hz, 3H), 2.29-2.14 (m, 6H), 2.12-1.99 (m, 6H), 1.81-1.73 (m, 1H), 1.60 (s, 4H), 1.44 (dd, J=2.8, 6.4 Hz, 1H), 1.36 (d, J=7.2 Hz, 2H), 0.98-0.92 (m, 3H), 0.83-0.75 (m, 6H).

Exemplary Synthesis of [(3S,8R)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 331)

Step 1: Preparation of [(3S,8R)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methoxy-tert-butyl-diphenyl-silane

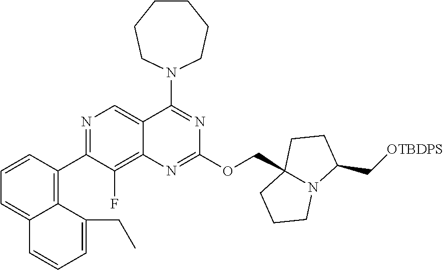

To a solution of 4-(azepan-1-yl)-7-(8-ethyl-1-naphthyl)-8-fluoro-2-methylsulfonyl-pyrido[4,3-d]pyrimidine (333 mg, 0.70 mmol, 1 eq) and [(3S,8R)-3-[[tert-butyl(diphenyl)silyl]oxymethyl]-1,2,3,5,6,7-hexahydropyrrolizin-8-yl]methanol (342 mg, 0.83 mmol, 1.2 eq) in tetrahydrofuran (10 mL) at 0° C. under nitrogen was added lithium tert-butoxide (2.2 M, 1.02 mL, 3.21 eq), and the reaction mixture was warmed to 25° C. and stirred at 25° C. for 0.5 h. The mixture was poured into water (20 mL) and stirred for 1 minute. The aqueous phase was extracted with ethyl acetate (20 mL×3), and the combined organic extract was washed with brine (20 mL×2), dried with anhydrous sodium sulfate, filtered, and concentrated. The residue was purified by flash silica gel chromatography (gradient: 0˜50% Ethyl acetate/Petroleum ether) to afford [(3S,8R)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methoxy-tert-butyl-diphenyl-silane (508 mg, 0.70 mmol, 90% yield) as a yellow oil. LC/MS (ESI) m/z: 808.3 [M+H]⁺.

Step 2: Preparation of [(3S,8R)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methanol

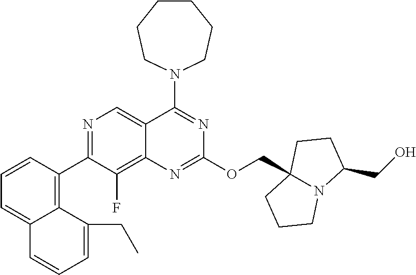 [SMILES: CCc1cccc2cccc(-c3ncc4c(N5CCCCCC5)nc(OC[C@]56CCCN5[C@H](CO)CC6)nc4c3F)c12]

To a solution of [(3S,8R)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methoxy-tert-butyl-diphenyl-silane (500 mg, 0.62 mmol, 1 eq) in DMF (2 mL) was added cesium fluoride (1.88 g, 12.37 mmol, 0.5 mL, 20 eq), and the reaction mixture was stirred at 25° C. for 16 h. The mixture was poured into water (20 mL) and stirred for 2 minutes. The aqueous phase was extracted with ethyl acetate (20 mL×3), and the combined organic extract was washed with brine (20 mL×2), dried over anhydrous sodium sulfate, filtered, and concentrated. The residue was purified by prep-HPLC (column: Phenomenex luna C18 150*40 mm*15 um; mobile phase: [22-52% CH₃CN in water (formic acid)]) to afford [(3S,8R)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methanol (100 mg, 0.18 mmol, 28% yield) as a white solid. LC/MS (ESI) m/z: 570.2 [M+H]⁺. ¹HNMR (400 MHz, DMSO-d₆) δ 9.14 (s, 1H), 8.11-8.03 (m, 1H), 7.91 (d, J=8.0 Hz, 1H), 7.62-7.55 (m, 1H), 7.51 (t, J=7.6 Hz, 1H), 7.41 (dd, J=6.4, 17.2 Hz, 1H), 7.46-7.34 (m, 1H), 4.57-4.26 (m, 1H), 4.16-4.09 (m, 1H), 4.02-3.92 (m, 1H), 4.04-3.88 (m, 4H), 3.28-3.23 (m, 1H), 2.92 (d, J=4.4 Hz, 1H), 2.72 (d, J=4.8 Hz, 2H), 2.41-2.26 (m, 2H), 2.08-2.01 (m, 1H), 1.97-1.90 (m, 5H), 1.85-1.75 (m, 3H), 1.60 (s, 7H), 1.23 (s, 1H), 0.85 (t, J=7.2 Hz, 3H).

Step 3: Preparation of [(3S,8R)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl 4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

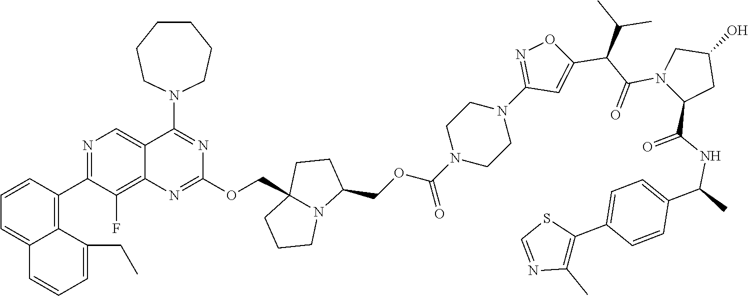 [SMILES: CCc1cccc2cccc(-c3ncc4c(N5CCCCCC5)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was made in an analogous manner to [(3R,8S)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from [(3S,8R)-8-[[4-(azepan-1-yl)-7-(8-ethyl-1-naphthyl)-8-fluoro-pyrido[4,3-d]pyrimidin-2-yl]oxymethyl]-1,2,3,5,6,7-hexahydropyrrolizin-3-yl]methyl (4-nitrophenyl) carbonate and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid salt, white solid). LC/MS (ESI) m/z: 1162.4 [M+H]⁺. ¹H NMR (400 MHz, DMSO-d₆) δ 9.14 (s, 1H), 8.98 (s, 1H), 8.45-8.39 (m, 1H), 8.07 (d, J=7.8 Hz, 1H), 7.91 (d, J=8.0 Hz, 1H), 7.57 (dt, J=4.3, 7.6 Hz, 1H), 7.53-7.47 (m, 1H), 7.46-7.41 (m, 3H), 7.40-7.33 (m, 3H), 6.19-6.04 (m, 1H), 4.95-4.86 (m, 1H), 4.35 (t, J=8.0 Hz, 1H), 4.28 (s, 1H), 4.15-4.09 (m, 1H), 4.06-3.92 (m, 6H), 3.90-3.83 (m, 1H), 3.71 (dd, J=4.5, 10.4 Hz, 1H), 3.58 (d, J=9.6 Hz, 1H), 3.44 (d, J=3.2 Hz, 6H), 3.15 (s, 4H), 2.97-2.87 (m, 2H), 2.55-2.52 (m, 3H), 2.45 (s, 3H), 2.26-2.20 (m, 1H), 2.09-1.99 (m, 2H), 1.93 (s, 5H), 1.84-1.75 (m, 4H), 1.70-1.53 (m, 7H), 1.37 (d, J=7.2 Hz, 3H), 0.98-0.92 (m, 3H), 0.84 (t, J=7.2 Hz, 3H), 0.78 (d, J=6.8 Hz, 3H).

Exemplary Synthesis of ((3S,7aR)-7a-(((4-((1R,5S)-3,8-diazabicyclo[3.2.1]octan-3-yl)-7-(8-chloro-3-hydroxynaphthalen-1-yl)-8-fluoropyrido[4,3-d]pyrimidin-2-yl)oxy)methyl) hexahydro-1H-pyrrolizin-3-yl)methyl 4-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperazine-1-carboxylate (Compound 268)

Step 1: Preparation of (1R,5S)-tert-butyl 3-(7-(8-chloro-3-(methoxy methoxy)naphthalen-1-yl)-8-fluoro-2-(methylthio)pyrido[4,3-d]pyrimidin-4-yl)-3,8-diazabicyclo[3.2.1]octane-8-carboxylate

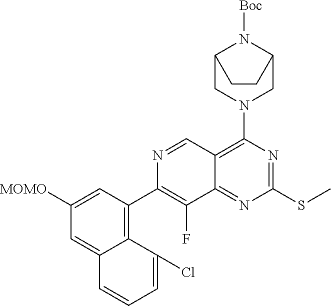

A mixture of (1R,5S)-tert-butyl 3-(7-chloro-8-fluoro-2-(methylthio)pyrido[4,3-d]pyrimidin-4-yl)-3,8-diazabicyclo[3.2.1]octane-8-carboxylate (1.59 g, 3.61 mmol, 0.9 eq), 2-(8-chloro-3-(methoxymethoxy)naphthalen-1-yl)-4,4,5,5-tetramethyl-1,3,2-dioxaborolane (1.4 g, 4.02 mmol, 1 eq), methanesulfonato (diadamantyl-n-butylphosphino)-2-amino-1,1-biphenyl-2-yl) palladium (II) (292 mg, 0.40 mmol, 0.1 eq), and potassium phosphate (2.13 g, 10.04 mmol, 2.5 eq) in dioxane (10 mL) and water (2 mL) was degassed and purged with nitrogen (3×), and the reaction mixture was stirred at 90° C. for 16 h under nitrogen atmosphere. The mixture was diluted with water (30 mL) and extracted with ethyl acetate (30 mL×2). The combined organic extracts were washed with brine (30 mL×2), dried over anhydrous sodium sulfate, filtered, and concentrated. The residue was purified by flash silica gel chromatography (gradient: 0˜25% Ethyl acetate/Petroleum ether) to afford (1R,5S)-tert-butyl 3-(7-(8-chloro-3-(methoxymethoxy)naphthalen-1-yl)-8-fluoro-2-(methylthio)pyrido[4,3-d]pyrimidin-4-yl)-3,8-diazabicyclo[3.2.1]octane-8-carboxylate (1.3 g, 2.08 mmol, 51% yield) as an orange oil. LC/MS (ESI) m/z: 626.4 [M+H]⁺.

Step 2: Preparation of (1R,5S)-tert-butyl 3-(7-(8-chloro-3-(methoxy methoxy)naphthalen-1-yl)-8-fluoro-2-(methylsulfonyl)pyrido[4,3-d]pyrimidin-4-yl)-3,8-diazabicyclo[3.2.1]octane-8-carboxylate

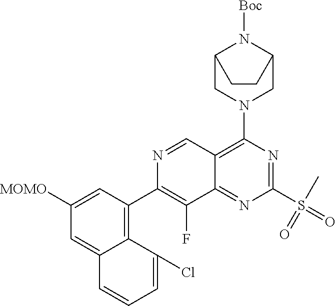

To a solution of (1R,5S)-tert-butyl 3-(7-(8-chloro-3-(methoxymethoxy)naphthalen-1-yl)-8-fluoro-2-(methylthio)pyrido[4,3-d]pyrimidin-4-yl)-3,8-diazabicyclo[3.2.1]octane-8-carboxylate (1 g, 1.60 mmol, 1 eq) in DMF (2 mL) was added 4 Å MS (1 g), and the resulting mixture was stirred at 25° C. for 4 h. Oxone (2.95 g, 4.79 mmol, 3 eq) was then added, and the reaction mixture was stirred at 25° C. for 12 h. The mixture was filtered, and the filter cake was washed with ethyl acetate (700 mL). The filtrate was diluted with brine (100 mL×2), dried over anhydrous sodium sulfate, filtered, and concentrated. The residue was purified by flash silica gel chromatography (gradient: 0˜35% Ethyl acetate/Petroleum ether) to afford (1R,5S)-tert-butyl 3-(7-(8-chloro-3-(methoxymethoxy)naphthalen-1-yl)-8-fluoro-2-(methylsulfonyl)pyrido[4,3-d]pyrimidin-4-yl)-3,8-diazabicyclo[3.2.1]octane-8-carboxylate (980 mg, 1.49 mmol, 93% yield) as a yellow solid. LC/MS (ESI) m/z: 658.2 [M+H]⁺.

Step 3: Preparation of (1R,5S)-tert-butyl 3-(2-(((3S,7aR)-3-(((tert-butyldiphenylsilyl)oxy)methyl) hexahydro-1H-pyrrolizin-7a-yl)methoxy)-7-(8-chloro-3-(methoxymethoxy)naphthalen-1-yl)-8-fluoropyrido[4,3-d]pyrimidin-4-yl)-3,8-diazabicyclo[3.2.1]octane-8-carboxylate

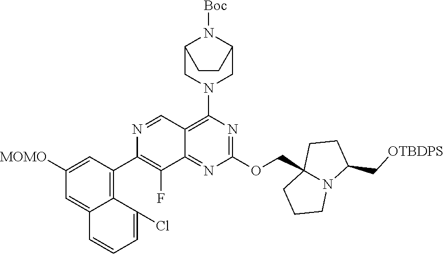

To a solution of (1R,5S)-tert-butyl 3-(7-(8-chloro-3-(methoxymethoxy)naphthalen-1-yl)-8-fluoro-2-(methylsulfonyl)pyrido[4,3-d]pyrimidin-4-yl)-3,8-diazabicyclo[3.2.1]octane-8-carboxylate (980 mg, 1.49 mmol, 1 eq) and ((3S,7aR)-3-(((tert-butyldiphenylsilyl)oxy)methyl) hexahydro-1H-pyrrolizin-7a-yl) methanol (793 mg, 1.94 mmol, 1.3 eq) in tetrahydrofuran (10 mL) at 0° C. was added lithium tert-butoxide (357 mg, 4.47 mmol, 0.4 mL, 3 eq), and the reaction mixture was stirred at 25° C. for 12 h. The mixture was diluted with water (30 mL) and extracted with ethyl acetate (30 mL). The organic suspension was filtered, and the filtrate was washed with brine (30 mL×2), dried over anhydrous sodium sulfate, filtered, and concentrated. The residue was purified by flash silica gel chromatography (gradient: 0˜34% Ethyl acetate/Petroleum ether) to afford (1R,5S)-tert-butyl 3-(2-(((3S,7aR)-3-(((tert-butyldiphenylsilyl)oxy)methyl) hexahydro-1H-pyrrolizin-7a-yl)methoxy)-7-(8-chloro-3-(methoxymethoxy)naphthalen-1-yl)-8-fluoropyrido[4,3-d]pyrimidin-4-yl)-3,8-diazabicyclo[3.2.1]octane-8-carboxylate (1.02 g, 1.03 mmol, 69% yield) as a yellow oil. LC/MS (ESI) m/z: 987.5 [M+H]⁺.

Step 4: Preparation of (1R,5S)-tert-butyl 3-(7-(8-chloro-3-(methoxy methoxy)naphthalen-1-yl)-8-fluoro-2-(((3S,7aR)-3-(hydroxymethyl) hexahydro-1H-pyrrolizin-7a-yl) methoxy)pyrido[4,3-d]pyrimidin-4-yl)-3,8-diazabicyclo[3.2.1]octane-8-carboxylate

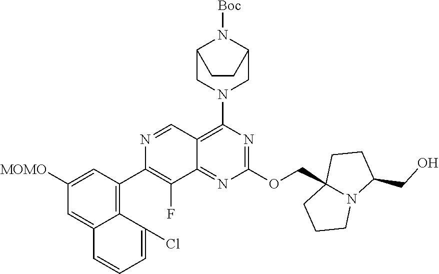

To a solution of (1R,5S)-tert-butyl 3-(2-(((3S,7aR)-3-(((tert-butyldiphenylsilyl)oxy)methyl) hexahydro-1H-pyrrolizin-7a-yl)methoxy)-7-(8-chloro-3-(methoxymethoxy)naphthalen-1-yl)-8-fluoropyrido[4,3-d]pyrimidin-4-yl)-3,8-diazabicyclo[3.2.1]octane-8-carboxylate (1.02 g, 1.03 mmol, 1 eq) in DMF (10 mL) was added cesium fluoride (2.35 g, 15.49 mmol, 15 eq), and the reaction mixture was stirred at 25° C. for 12 h. The mixture was diluted with water (50 mL) and extracted with ethyl acetate (30 mL×2). The combined organic extracts were washed with brine (30 mL×2), dried over anhydrous sodium sulfate, filtered, and concentrated. The residue was purified by prep-TLC (Dichloromethane/Methanol=8:1) to afford (1R,5S)-tert-butyl 3-(7-(8-chloro-3-(methoxymethoxy)naphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3,8-diazabicyclo[3.2.1]octane-8-carboxylate (283 mg, 0.38 mmol, 36% yield) as a yellow solid. LC/MS (ESI) m/z: 749.5 [M+H]⁺.

Step 5: Preparation of ((3S,7aR)-7a-(((4-((1R,5S)-3,8-diazabicyclo[3.2.1]octan-3-yl)-7-(8-chloro-3-hydroxynaphthalen-1-yl)-8-fluoropyrido[4,3-d]pyrimidin-2-yl)oxy)methyl) hexahydro-1H-pyrrolizin-3-yl)methyl 4-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperazine-1-carboxylate

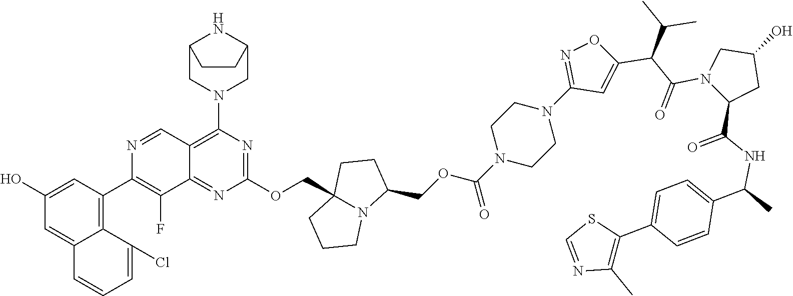 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cnc(-c8cc(O)cc9cccc(Cl)c89)c(F)c7n6)CCCN45)CC3)no2)C(C)C)cc1]

The title compound was made in an analogous manner to [(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl 6-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptane-2-carboxylate starting from (1R,5S)-tert-butyl 3-(7-(8-chloro-3-(methoxymethoxy)naphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3,8-diazabicyclo[3.2.1]octane-8-carboxylate and (2S,4R)-4-hydroxy-1-((R)-3-methyl-2-(3-(piperazin-1-yl)isoxazol-5-yl)butanoyl)-N—((S)-1-(4-(4-methylthiazol-5-yl)phenyl)ethyl)pyrrolidine-2-carboxamide. (formic acid salt, white solid). LC/MS (ESI) m/z: 599.9 [M/2+H]⁺. ¹H NMR (400 MHz, DMSO-d₆) δ 9.06 (s, 1H), 8.98 (s, 1H), 8.40 (d, J=7.6 Hz, 1H), 8.17 (s, 1H), 7.87-7.76 (m, 1H), 7.46-7.39 (m, 3H), 7.39-7.33 (m, 4H), 7.30-7.19 (m, 1H), 7.16-7.11 (m, 1H), 6.18-6.03 (m, 1H), 4.96-4.86 (m, 1H), 4.49-4.39 (m, 2H), 4.36 (t, J=8.0 Hz, 1H), 4.28 (d, J=2.4 Hz, 1H), 4.10-4.02 (m, 2H), 3.96-3.91 (m, 1H), 3.90-3.83 (m, 2H), 3.73-3.68 (m, 2H), 3.65-3.56 (m, 8H), 3.15 (d, J=3.6 Hz, 5H), 2.97-2.87 (m, 3H), 2.71-2.63 (m, 1H), 2.45 (s, 3H), 2.27-2.15 (m, 1H), 2.07-1.92 (m, 3H), 1.86-1.74 (m, 4H), 1.72-1.48 (m, 7H), 1.41-1.31 (m, 3H), 0.99-0.90 (m, 3H), 0.84-0.74 (m, 3H).

Exemplary Synthesis of ((3S,7aR)-7a-(((4-((1R,5S)-3,8-diazabicyclo[3.2.1]octan-3-yl)-7-(7,8-difluoronaphthalen-1-yl)-8-fluoropyrido[4,3-d]pyrimidin-2-yl)oxy)methyl) hexahydro-1H-pyrrolizin-3-yl)methyl 4-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperazine-1-carboxylate (Compound 269)

Step 1: Preparation of (1R,5S)-tert-butyl 3-(2-(((3S,7aR)-3-(((tert-butyldiphenylsilyl)oxy)methyl) hexahydro-1H-pyrrolizin-7a-yl)methoxy)-7-(7,8-difluoronaphthalen-1-yl)-8-fluoropyrido[4,3-d]pyrimidin-4-yl)-3,8-diazabicyclo[3.2.1]octane-8-carboxylate

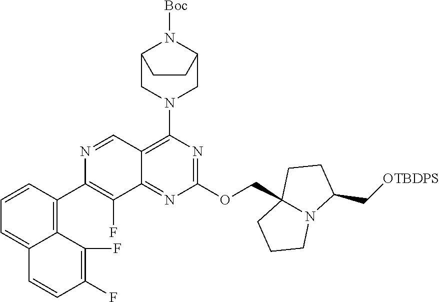

To a solution of (1R,5S)-tert-butyl 3-(7-(7,8-difluoronaphthalen-1-yl)-8-fluoro-2-(methylsulfonyl)pyrido[4,3-d]pyrimidin-4-yl)-3,8-diazabicyclo[3.2.1]octane-8-carboxylate (800 mg, 1.33 mmol, 1 eq) and ((3S,7aR)-3-(((tert-butyldiphenylsilyl)oxy)methyl) hexahydro-1H-pyrrolizin-7a-yl) methanol (710 mg, 1.73 mmol, 1.3 eq) in tetrahydrofuran (8 mL) at 0° C. was added lithium tert-butoxide (320 mg, 4.00 mmol, 0.36 mL, 3 eq), and the reaction mixture was stirred at 25° C. for 12 h. The mixture was diluted with water (30 mL) and extracted with EtOAc (30 mL). The organic suspension was filtered, and the filtrate was washed with brine (30 mL×2), dried over anhydrous sodium sulfate, filtered, and concentrated. The residue was purified by flash silica gel chromatography (gradient: 0˜33% Ethyl acetate/Petroleum ether) to afford (1R,5S)-tert-butyl 3-(2-(((3S,7aR)-3-(((tert-butyldiphenylsilyl)oxy)methyl) hexahydro-1H-pyrrolizin-7a-yl)methoxy)-7-(7,8-difluoronaphthalen-1-yl)-8-fluoropyrido[4,3-d]pyrimidin-4-yl)-3,8-diazabicyclo[3.2.1]octane-8-carboxylate (1.11 g, 1.19 mmol, 90% yield) as a yellow oil. LC/MS (ESI) m/z: 930.6 [M+H]⁺.

Step 2: Preparation of (1R,5S)-tert-butyl 3-(7-(7,8-difluoronaphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3,8-diazabicyclo[3.2.1]octane-8-carboxylate

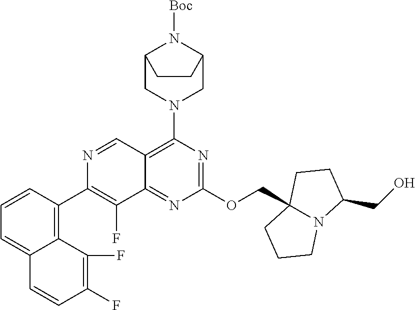 [SMILES: CC(C)(C)OC(=O)N1C2CCC1CN(c1nc(OC[C@]34CCCN3[C@H](CO)CC4)nc3c(F)c(-c4cccc5ccc(F)c(F)c45)ncc13)C2]

To a solution of (1R,5S)-tert-butyl 3-(2-(((3S,7aR)-3-(((tert-butyldiphenylsilyl)oxy)methyl) hexahydro-1H-pyrrolizin-7a-yl)methoxy)-7-(7,8-difluoronaphthalen-1-yl)-8-fluoropyrido[4,3-d]pyrimidin-4-yl)-3,8-diazabicyclo[3.2.1]octane-8-carboxylate (1.11 g, 1.19 mmol, 1 eq) in DMF (11 mL) was added cesium fluoride (2.72 g, 17.92 mmol, 15 eq), and the reaction mixture was stirred at 25° C. for 12 h. The mixture was diluted with water (50 mL) and extracted with ethyl acetate (30 mL×2). The combined organic extracts were washed with brine (30 mL×2), dried over anhydrous sodium sulfate, filtered and concentrated. The residue was purified by prep-TLC (Dichloromethane/Methanol=8:1) to afford (1R,5S)-tert-butyl 3-(7-(7,8-difluoronaphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3,8-diazabicyclo[3.2.1]octane-8-carboxylate (347 mg, 0.5 mmol, 42% yield) as a white solid. LC/MS (ESI) m/z: 691.4 [M+H]⁺.

Step 3: Preparation of ((3S,7aR)-7a-(((4-((1R,5S)-3,8-diazabicyclo[3.2.1]octan-3-yl)-7-(7,8-difluoronaphthalen-1-yl)-8-fluoropyrido[4,3-d]pyrimidin-2-yl)oxy)methyl) hexahydro-1H-pyrrolizin-3-yl)methyl 4-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperazine-1-carboxylate

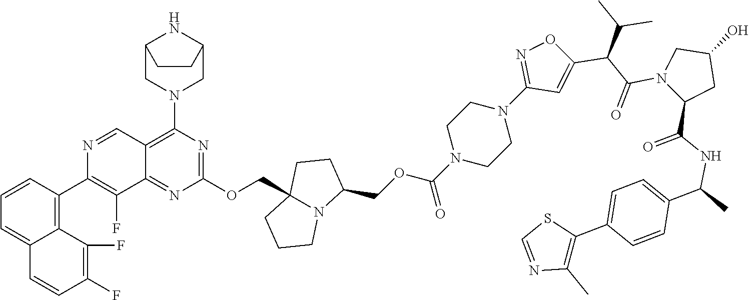 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cnc(-c8cccc9ccc(F)c(F)c89)c(F)c7n6)CCCN45)CC3)no2)C(C)C)cc1]

The title compound was prepared in an analogous manner to [(3S,8R)-8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-1,2,3,5,6,7-hexahydropyrrolizin-3-yl]methyl4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from (1R,5S)-tert-butyl 3-(7-(7,8-difluoronaphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3,8-diazabicyclo[3.2.1]octane-8-carboxylate and (2S,4R)-4-hydroxy-1-((R)-3-methyl-2-(3-(piperazin-1-yl)isoxazol-5-yl)butanoyl)-N—((S)-1-(4-(4-methylthiazol-5-yl)phenyl)ethyl)pyrrolidine-2-carboxamide. (formic acid salt, white solid). LC/MS (ESI) m/z: 592.5 [M/2+H]⁺. ¹H NMR (400 MHz, DMSO-d₆) δ 9.12 (s, 1H), 8.98 (s, 1H), 8.44-8.34 (m, 1H), 8.23-8.16 (m, 2H), 8.01 (dd, J=5.4, 8.0 Hz, 1H), 7.77-7.68 (m, 3H), 7.47-7.41 (m, 2H), 7.40-7.31 (m, 2H), 6.18-6.02 (m, 1H), 4.91 (br t, J=7.3 Hz, 1H), 4.50-4.41 (m, 2H), 4.40-4.32 (m, 1H), 4.32-4.24 (m, 1H), 4.14-4.00 (m, 2H), 3.98-3.84 (m, 3H), 3.75-3.68 (m, 2H), 3.68-3.56 (m, 9H), 3.15 (br s, 6H), 2.96-2.88 (m, 2H), 2.71-2.62 (m, 1H), 2.45 (s, 3H), 2.28-2.10 (m, 2H), 2.09-1.88 (m, 3H), 1.86-1.73 (m, 4H), 1.72-1.61 (m, 5H), 1.59-1.49 (m, 1H), 1.37 (d, J=6.8 Hz, 2H), 1.00-0.90 (m, 3H), 0.84-0.74 (m, 3H).

Exemplary Synthesis of [(3S,8R)-8-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 258)

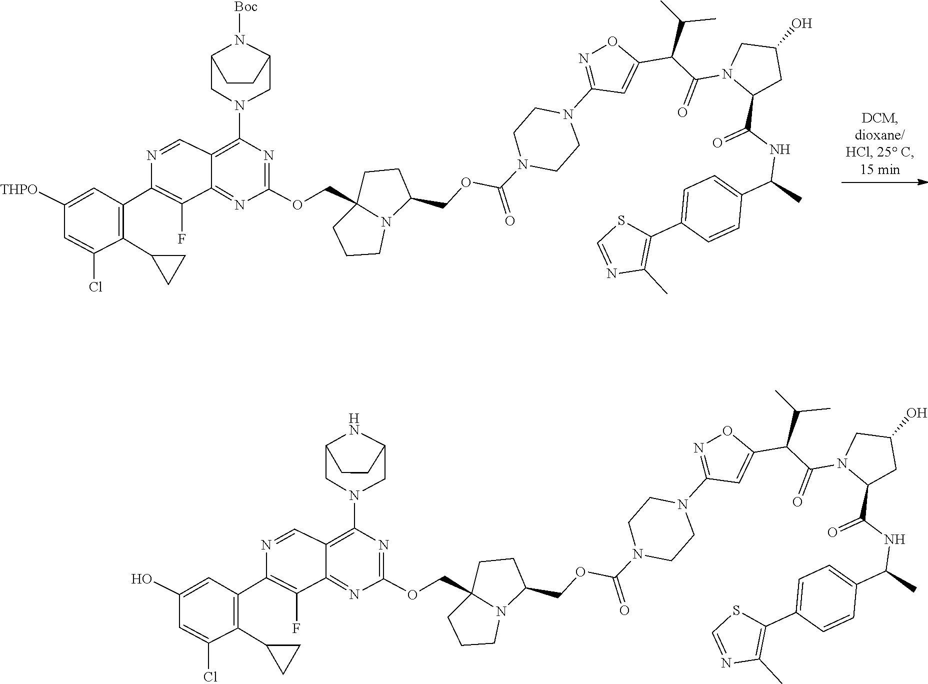

The title compound was made in an analogous manner to [(3S,8R)-8-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from tert-butyl 3-[7-[3-chloro-2-cyclopropyl-5-(methoxymethoxy)phenyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid, white solid). LC/MS (ESI) m/z: 1187.8 [M+H]⁺. ¹H NMR (400 MHz, DMSO-d₆) δ 9.08 (s, 1H), 8.98 (s, 1H), 8.40 (d, J=7.6 Hz, 1H), 8.24 (s, 1H), 7.45-7.42 (m, 2H), 7.39-7.34 (m, 2H), 6.95 (d, J=2.4 Hz, 1H), 6.77 (d, J=2.4 Hz, 1H), 6.16 (s, 1H), 4.91 (t, J=7.2 Hz, 1H), 4.45-4.32 (m, 3H), 4.28 (s, 1H), 4.11-4.05 (m, 1H), 4.04-3.99 (m, 1H), 3.97-3.91 (m, 1H), 3.87 (dd, J=6.4, 10.4 Hz, 1H), 3.71 (dd, J=4.0, 10.4 Hz, 1H), 3.62-3.55 (m, 4H), 3.50-3.43 (s, 5H), 3.16 (s, 5H), 2.96-2.90 (m, 2H), 2.71-2.62 (m, 1H), 2.54 (s, 1H), 2.45 (s, 3H), 2.28-2.11 (m, 2H), 2.06-1.90 (m, 3H), 1.86-1.74 (m, 5H), 1.71-1.53 (m, 7H), 1.37 (d, J=7.2 Hz, 3H), 0.99-0.92 (m, 3H), 0.83-0.75 (m, 3H), 0.66-0.47 (m, 2H), 0.02-0.09 (m, 2H).

Exemplary Synthesis of [(3S,8R)-8-[[7-[3-chloro-5-hydroxy-2-(trifluoromethyl)phenyl]-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 257)

Step 1: Preparation of tert-butyl 3-[7-[3-chloro-5-(methoxymethoxy)-2-(trifluoromethyl)phenyl]-8-fluoro-2-methylsulfanyl-pyrido[4,3-d]pyrimidin-4-yl]-3,8-diazabicyclo[3.2.1]octane-8-carboxylate

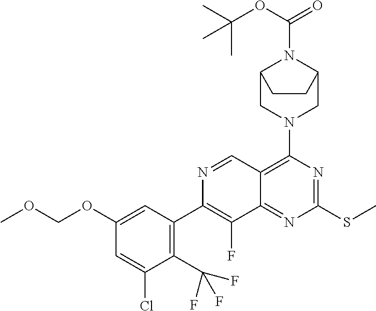

To a mixture of tert-butyl 3-(7-chloro-8-fluoro-2-methylsulfanyl-pyrido[4,3-d]pyrimidin-4-yl)-3,8-diazabicyclo[3.2.1]octane-8-carboxylate (1.0 g, 2.27 mmol, 1 eq) and 2-[3-chloro-5-(methoxymethoxy)-2-(trifluoromethyl)phenyl]-4,4,5,5-tetramethyl-1,3,2-dioxaborolane (1.25 g, 3.41 mmol, 1.5 eq) in dioxane (15 mL) and water (3 mL) were added potassium phosphate (1.45 g, 6.82 mmol, 3 eq) and methanesulfonato (diadamantyl-n-butylphosphino)-2′-amino-1,1′-biphenyl-2-yl) palladium (II) (165 mg, 0.23 mmol, 0.1 eq) under nitrogen, and the reaction mixture was stirred at 80° C. for 16 h. The mixture was cooled to 25° C., then poured into water (20 mL) and stirred for 1 minute. The aqueous phase was extracted with ethyl acetate (10 mL×3), and the combined organic extract was washed with brine (10 mL×2), dried over anhydrous sodium sulfate, filtered, and concentrated. The residue was purified by flash silica gel chromatography (gradient: 0˜25% Ethyl acetate/Petroleum ether) to afford tert-butyl 3-[7-[3-chloro-5-(methoxymethoxy)-2-(trifluoromethyl)phenyl]-8-fluoro-2-methylsulfanyl-pyrido[4,3-d]pyrimidin-4-yl]-3,8-diazabicyclo[3.2.1]octane-8-carboxylate (750 mg, 1.16 mmol, 51% yield) as a white solid. LC/MS (ESI) m/z: 644.2 [M+H]⁺.

Step 2: Preparation of tert-butyl 3-[7-[3-chloro-5-(methoxymethoxy)-2-(trifluoromethyl)phenyl]-8-fluoro-2-methylsulfonyl-pyrido[4,3-d]pyrimidin-4-yl]-3,8-diazabicyclo[3.2.1]octane-8-carboxylate

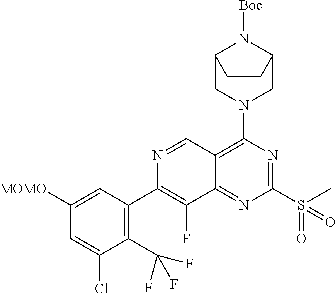

To a solution of tert-butyl 3-[7-[3-chloro-5-(methoxymethoxy)-2-(trifluoromethyl)phenyl]-8-fluoro-2-methylsulfanyl-pyrido[4,3-d]pyrimidin-4-yl]-3,8-diazabicyclo[3.2.1]octane-8-carboxylate (750 mg, 1.16 mmol, 1 eq) in DMF (15 mL) was added molecular sieves (4 Å, 1.5 g), and the resulting mixture was stirred for 1 h. Oxone (2.86 g, 4.66 mmol, 4 eq) was then added, and the reaction mixture was stirred at 25° C. for 11 h. The mixture was filtrated and concentrated, and the resulting residue was poured into water (100 mL) and stirred for 2 minutes. The aqueous phase was extracted with ethyl acetate (50 mL×2), and the combined organic extract was washed with brine (50 mL×2), dried over anhydrous sodium sulfate, filtered, and concentrated. The residue was purified by prep-TLC (petroleum ether:ethyl acetate=1:1) to afford tert-butyl 3-[7-[3-chloro-5-(methoxymethoxy)-2-(trifluoromethyl)phenyl]-8-fluoro-2-methylsulfonyl-pyrido[4,3-d]pyrimidin-4-yl]-3,8-diazabicyclo[3.2.1]octane-8-carboxylate (400 mg, 0.59 mmol, 51% yield) as a white solid. LC/MS (ESI) m/z: 676.3 [M+H]⁺.

Step 3: Preparation of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[3-chloro-5-(methoxymethoxy)-2-(trifluoromethyl)phenyl]-8-fluoro-pyrido[4,3-d]pyrimidin-4-yl]-3,8-diazabicyclo[3.2.1]octane-8-carboxylate

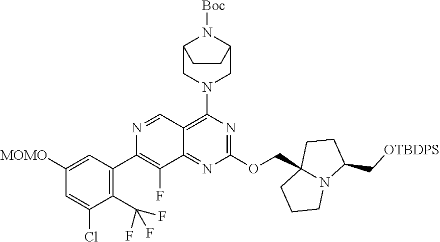

To a solution of tert-butyl 3-[7-[3-chloro-5-(methoxymethoxy)-2-(trifluoromethyl)phenyl]-8-fluoro-2-methylsulfonyl-pyrido[4,3-d]pyrimidin-4-yl]-3,8-diazabicyclo[3.2.1]octane-8-carboxylate (400 mg, 0.59 mmol, 1 eq) and [(3S,8R)-3-[[tert-butyl(diphenyl)silyl]oxymethyl]-1,2,3,5,6,7-hexahydropyrrolizin-8-yl]methanol (291 mg, 0.71 mmol, 1.2 eq) in tetrahydrofuran (5 mL) at 0° C. under nitrogen was added lithium tert-butoxide (2.2 M, 0.8 mL, 3 eq), and the reaction mixture was stirred at 25° C. for 2 h. The mixture was warmed to 20° C., then poured into water (20 mL) and stirred for 1 minute. The aqueous phase was extracted with ethyl acetate (20 mL×3), and the combined organic extract was washed with brine (20 mL×2), dried over anhydrous sodium sulfate, filtered, and concentrated in vacuum. The residue was purified by flash silica gel chromatography (gradient: 0˜40% Ethyl acetate/Petroleum ether) to afford tert-butyl 3-[2-[[(3S,8R)-3-[[tert-butyl(diphenyl)silyl]oxymethyl]-1,2,3,5,6,7-hexahydropyrrolizin-8-yl]methoxy]-7-[3-chloro-5-(methoxymethoxy)-2-(trifluoromethyl)phenyl]-8-fluoro-pyrido[4,3-d]pyrimidin-4-yl]-3,8-diazabicyclo[3.2.1]octane-8-carboxylate (360 mg, 0.35 mmol, 60% yield) as a white solid. LC/MS (ESI) m/z: 1005.4 [M+H]⁺.

Step 4: Preparation of tert-butyl 3-[7-[3-chloro-5-(methoxymethoxy)-2-(trifluoromethyl)phenyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

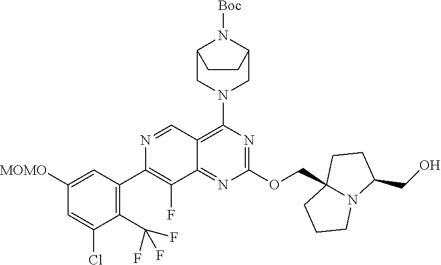

To a solution of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[3-chloro-5-(methoxymethoxy)-2-(trifluoromethyl)phenyl]-8-fluoro-pyrido[4,3-d]pyrimidin-4-yl]-3,8-diazabicyclo[3.2.1]octane-8-carboxylate (406 mg, 0.40 mmol, 1 eq) in DMF (5 mL) was added cesium fluoride (1.23 g, 8.07 mmol, 0.3 mL, 20 eq), and the reaction mixture was stirred at 25° C. for 12 h. The mixture was poured into water (50 mL) and stirred for 2 minutes. The aqueous phase was extracted with dichloromethane (20 mL×2), and the combined organic extract was washed with brine (20 mL×3), dried over anhydrous sodium sulfate, filtered, and concentrated. The residue was purified by prep-TLC (dichloromethane:methanol=10:1) to afford tert-butyl 3-[7-[3-chloro-5-(methoxymethoxy)-2-(trifluoromethyl)phenyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (306 mg, 0.40 mmol, 99% yield) as a white solid. LC/MS (ESI) m/z: 767.5 [M+H]⁺.

Step 5: Preparation of [(3S,8R)-8-[[7-[3-chloro-5-hydroxy-2-(trifluoromethyl)phenyl]-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

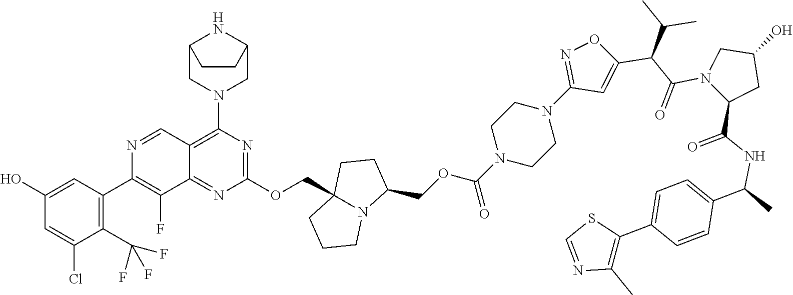 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cnc(-c8cc(O)cc(Cl)c8C(F)(F)F)c(F)c7n6)CCCN45)CC3)no2)C(C)C)cc1]

The title compound was made in an analogous manner to [(3S,8R)-8-[[7-(3-chloro-2-cyclopropyl-5-hydroxy-phenyl)-4-(3,8-diazabicyclo[3.2.1]octan-3-yl)-8-fluoro-pyrido[4,3-d]pyrimidin-2-yl]oxymethyl]-1,2,3,5,6,7-hexahydropyrrolizin-3-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate starting from tert-butyl 3-[7-[3-chloro-5-(methoxymethoxy)-2-(trifluoromethyl)phenyl]-8-fluoro-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid, white solid). LC/MS (ESI) m/z: 1215.4 [M+H]⁺. ¹H NMR (400 MHz, DMSO-d₆) δ 9.06 (s, 1H), 8.98 (s, 1H), 8.40 (d, J=7.6 Hz, 1H), 8.19 (s, 2H), 7.45-7.42 (m, 2H), 7.38-7.35 (m, 2H), 7.18 (d, J=2.0 Hz, 1H), 6.79 (d, J=1.2 Hz, 1H), 6.16 (s, 1H), 4.91 (t, J=7.2 Hz, 1H), 4.48-4.32 (m, 4H), 4.28 (s, 1H), 4.11-4.00 (m, 3H), 3.96-3.86 (m, 3H), 3.64 (s, 4H), 3.16 (s, 5H), 2.92 (d, J=4.0 Hz, 2H), 2.54 (s, 1H), 2.45 (s, 3H), 2.26-2.14 (m, 2H), 2.06-1.89 (m, 4H), 1.82-1.76 (m, 4H), 1.70-1.52 (m, 9H), 1.37 (d, J=7.2 Hz, 3H), 0.95 (d, J=6.4 Hz, 3H), 0.78 (d, J=6.8 Hz, 3H).

Exemplary Synthesis of [(3S,8S)-3-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-8-yl]methyl 4-[5-[(1R)-1-[(2S,4R)-4-hydr oxy-2-[[(1S)-1-[4-(4-methylthiazol-5-yl)phenyl]ethyl]carbamoyl]pyrrolidine-1-carbonyl]-isobutyl]isoxazol-3-yl]piperazine-1-carboxylate (Compound 342)

Step 1: Preparation of tert-butyl 3-[2-[[(3S,8S)-8-[[tert-butyl(diphenyl)silyl]oxymethyl]-1,2,3,5,6,7-hexahydropyrrolizin-3-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

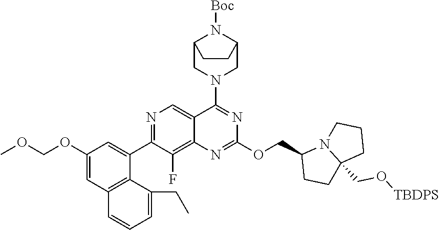

To a yellow suspension of tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (400 mg, 0.614 mmol, 1.0 eq) and [(3S,8S)-8-[[tert-butyl(diphenyl)silyl]oxymethyl]-1,2,3,5,6,7-hexahydropyrrolizin-3-yl]methanol (302 mg, 0.736 mmol, 1.2 eq) in toluene (16 mL) was added 4 Å MS (700 mg), and the mixture was stirred at 35° C. for 1 hour. The mixture was cooled to 0° C., sodium tert-butoxide (147 mg, 1.53 mmol, 2.5 eq) was then added, and the reaction mixture was stirred at 0° C. for 30 minutes. The reaction was quenched with saturated aqueous NH₄Cl (2.0 mL), dried over anhydrous sodium sulfate, filtered, and concentrated to give tert-butyl 3-[2-[[(3S,8S)-8-[[tert-butyl(diphenyl)silyl]oxymethyl]-1,2,3,5,6,7-hexahydropyrrolizin-3-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (600 mg, crude) as a yellow gum. LC/MS (ESI) m/z: 981.6 [M+H]⁺.

Step 2: Preparation of tert-butyl 3-[7-[8-ethyl-3-(methoxy methoxy)-1-naphthyl]-8-fluoro-2-[[(3S,8S)-8-(hydroxymethyl)-1,2,3,5,6,7-hexahydropyrrolizin-3-yl]methoxy]pyrido[4,3-d]pyrimidin-4-yl]-3,8-diazabicyclo[3.2.1]octane-8-carboxylate

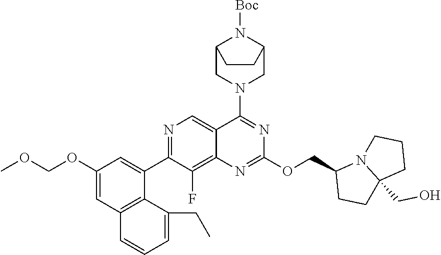

To a solution of tert-butyl 3-[2-[[(3S,8S)-8-[[tert-butyl(diphenyl)silyl]oxymethyl]-1,2,3,5,6,7-hexahydropyrrolizin-3-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (600 mg, 0.611 mmol theoretically) in DMF (8.0 mL) at 30° C. was added CsF (650 mg, 4.28 mmol), and the reaction mixture was stirred at 30° C. for 12 hours. Additional CsF (650 mg, 4.28 mmol) was added, and the reaction mixture was stirred at 40° C. for 24 hours. The mixture was diluted with water (15 mL) and extracted with ethyl acetate (3×20 mL). The combined extracts were washed with brine (2×20 mL), dried over anhydrous Na₂SO₄, filtered, and concentrated. The residue was purified by flash chromatography on SiO₂ (gradient: 0˜10% methanol in dichloromethane) to afford tert-butyl 3-[7-[8-ethyl-3-(methoxy methoxy)-1-naphthyl]-8-fluoro-2-[[(3S,8S)-8-(hydroxymethyl)-1,2,3,5,6,7-hexahydropyrrolizin-3-yl]methoxy]pyrido[4,3-d]pyrimidin-4-yl]-3,8-diazabicyclo[3.2.1]octane-8-carboxylate (307 mg, 0.388 mmol, 64% yield) as a yellow solid. LC/MS (ESI) m/z: 743.3 [M+H]⁺. ¹H NMR (400 MHz, CDCl₃) δ 9.03 (s, 1H), 7.71 (d, J=7.6 Hz, 1H), 7.56-7.52 (m, 1H), 7.45-7.38 (m, 1H), 7.26-7.22 (m, 1H), 7.20 (d, J=2.4 Hz, 1H), 5.34-5.28 (m, 2H), 4.75 (d, J=4.4 Hz, 2H), 4.65-4.36 (m, 4H), 3.84-3.61 (m, 3H), 3.60-3.44 (m, 5H), 3.39-3.19 (m, 1H), 3.11-2.94 (m, 1H), 2.41-2.29 (m, 2H), 2.11 (d, J=13.2 Hz, 1H), 2.03-1.89 (m, 6H), 1.87-1.73 (m, 4H), 1.72-1.65 (m, 1H), 1.53 (s, 9H), 0.95 (t, J=7.2 Hz, 3H).

Step 3: Preparation of tert-butyl 3-[7-[8-ethyl-3-(methoxy methoxy)-1-naphthyl]-8-fluoro-2-[[(3S,8S)-8-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]piperazine-1-carbonyl]oxymethyl]-1,2,3,5,6,7-hexahydropyrrolizin-3-yl]methoxy]pyrido[4,3-d]pyrimidin-4-yl]-3,8-diazabicyclo[3.2.1]octane-8-carboxylate

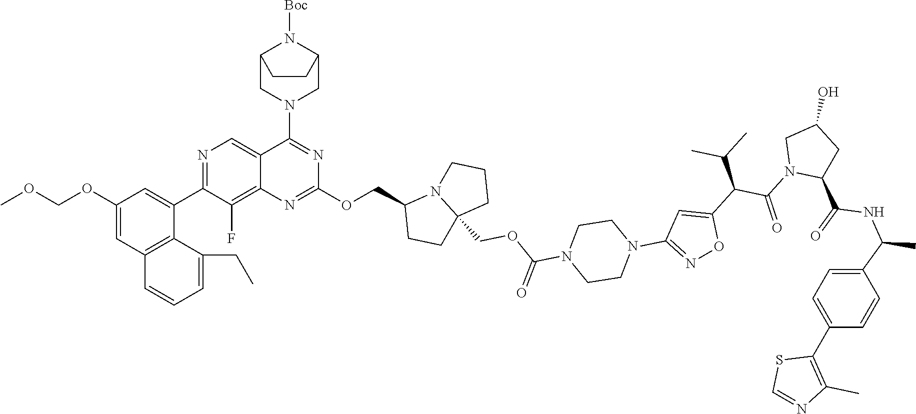 [SMILES: CCc1cccc2cc(OCOC)cc(-c3ncc4c(N5CC6CCC(C5)N6C(=O)OC(C)(C)C)nc(OC[C@@H]5CC[C@]6(COC(=O)N7CCN(c8cc([C@H](C(=O)N9C[C@H](O)C[C@H]9C(=O)N[C@@H](C)c9ccc(-c%10scnc%10C)cc9)C(C)C)on8)CC7)CCCN56)nc4c3F)c12]

To tert-butyl 3-[7-[8-ethyl-3-(methoxymethoxy)-1-naphthyl]-8-fluoro-2-[[(3S,8S)-8-(hydroxymethyl)-1,2,3,5,6,7-hexahydropyrrolizin-3-yl]methoxy]pyrido[4,3-d]pyrimidin-4-yl]-3,8-diazabicyclo[3.2.1]octane-8-carboxylate (200 mg, 94% purity, 0.253 mmol, 1.0 eq) in THF (6 mL) were added triethylamine (0.187 mL, d=0.727 g/mL, 1.35 mmol, 5.34 eq), DMAP (10 mg, 0.081 mmol, 0.32 eq), and (4-nitrophenyl) chloroformate (98 mg, 0.485 mmol, 1.8 eq), and the resulting mixture was stirred at 35° C. for 12 hours. (2S,4R)-4-Hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide (214 mg, 0.377 mmol, 1.49 eq) was then added, and the reaction mixture was stirred at 35° C. for 1 more hour. The mixture was concentrated, and the resulting residue was purified by flash chromatography on SiO₂ (gradient 0˜5% methanol in dichloromethane) to afford tert-butyl 3-[7-[8-ethyl-3-(methoxymethoxy)-1-naphthyl]-8-fluoro-2-[[(3S,8S)-8-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]piperazine-1-carbonyl]oxymethyl]-1,2,3,5,6,7-hexahydropyrrolizin-3-yl]methoxy]pyrido[4,3-d]pyrimidin-4-yl]-3,8-diazabicyclo[3.2.1]octane-8-carboxylate (301 mg, 0.194 mmol, 77% yield) as a yellow solid. LC/MS (ESI) m/z: 1335.7 [M+H]⁺.

Step 4: Preparation of [(3S,8S)-3-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-8-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]piperazine-1-carboxylate

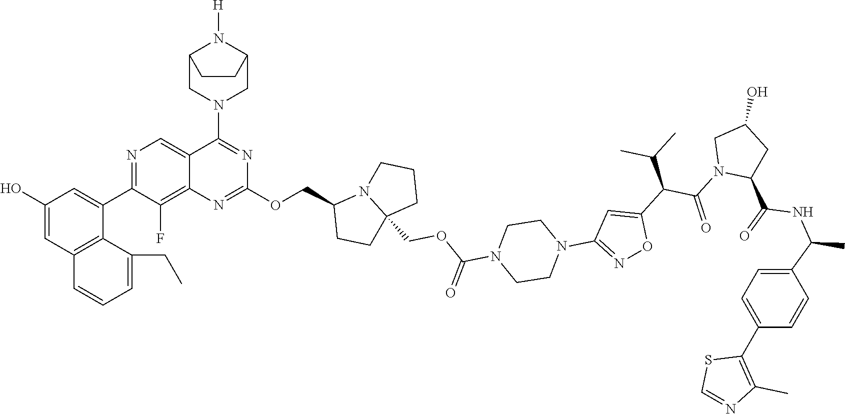 [SMILES: CCc1cccc2cc(O)cc(-c3ncc4c(N5CC6CCC(C5)N6)nc(OC[C@@H]5CC[C@]6(COC(=O)N7CCN(c8cc([C@H](C(=O)N9C[C@H](O)C[C@H]9C(=O)N[C@@H](C)c9ccc(-c%10scnc%10C)cc9)C(C)C)on8)CC7)CCCN56)nc4c3F)c12]

To tert-butyl 3-[7-[8-ethyl-3-(methoxymethoxy)-1-naphthyl]-8-fluoro-2-[[(3S,8S)-8-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]piperazine-1-carbonyl]oxymethyl]-1,2,3,5,6,7-hexahydropyrrolizin-3-yl]methoxy]pyrido[4,3-d]pyrimidin-4-yl]-3,8-diazabicyclo[3.2.1]octane-8-carboxylate (301 mg, 86% purity, 0.194 mmol) in dichloromethane (2.0 mL) was added 4 M HCl/dioxane (1.1 mL) in one portion at 20° C., and the reaction mixture was stirred at 20° C. for 30 minutes. The mixture was concentrated, and the residue was treated with triethylamine to pH=9. THF was then added, and the mixture was filtered and concentrated. The crude product was purified by preparative HPLC {Column: Phenomenex C18 75*30 mm*3 um; Mobile phase: [6-46% CH₃CN in water (formic acid)]}. Pure fraction was lyophilized to afford [(3S,8S)-3-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-8-yl]methyl 4-[5-[(1R)-1-[(2S,4R)-4-hydr oxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (132.5 mg, 0.105 mmol, 54% yield, formic acid salt) as a white solid. LC/MS (ESI) m/z: 1192.0 [M+H]⁺. ¹H NMR (400 MHz, CD₃OD) δ 9.15 (s, 1H), 8.90-8.84 (m, 1H), 8.45 (s, 2H), 7.68-7.59 (m, 1H), 7.48-7.32 (m, 5H), 7.31-7.27 (m, 1H), 7.17 (d, J=7.2 Hz, 1H), 7.01 (dd, J=6.8, 2.4 Hz, 1H), 6.16-6.04 (m, 1H), 5.06-4.97 (m, 2H), 4.82-4.66 (m, 4H), 4.53-4.45 (m, 1H), 4.44-4.31 (m, 2H), 4.22 (dd, J=12.0, 3.2 Hz, 1H), 4.15-4.03 (m, 1H), 4.02-3.94 (m, 2H), 3.92-3.72 (m, 3H), 3.66-3.49 (m, 7H), 3.41 (d, J=2.8 Hz, 1H), 3.25-3.13 (m, 3H), 2.51-2.44 (m, 3H), 2.40-2.14 (m, 9H), 2.09 (s, 1H), 2.05-1.89 (m, 7H), 1.58-1.44 (m, 3H), 1.08-0.99 (m, 3H), 0.91-0.83 (m, 6H).

Exemplary Synthesis of [(3R,8R)-3-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-8-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 343)

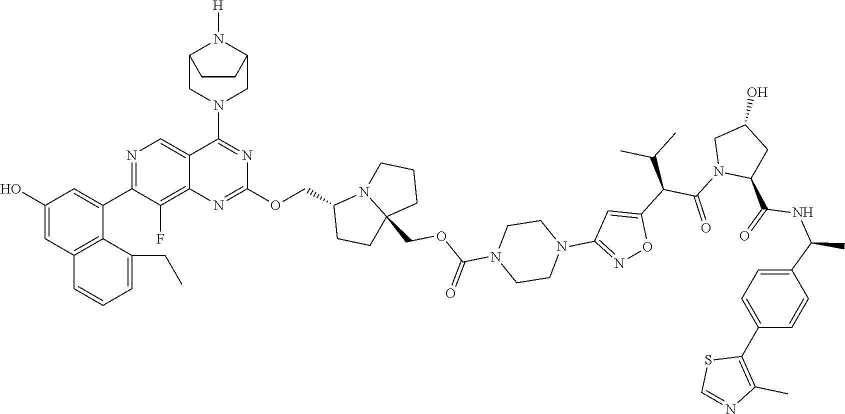 [SMILES: CCc1cccc2cc(O)cc(-c3ncc4c(N5CC6CCC(C5)N6)nc(OC[C@H]5CC[C@@]6(COC(=O)N7CCN(c8cc([C@H](C(=O)N9C[C@H](O)C[C@H]9C(=O)N[C@@H](C)c9ccc(-c%10scnc%10C)cc9)C(C)C)on8)CC7)CCCN56)nc4c3F)c12]

The title compound was made in an analogous manner to [(3S,8S)-3-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-8-yl]methyl 4-[5-[(1R)-1-[(2S,4R)-4-hydr oxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-methyl sulfonyl-pyrido[4,3-d]pyrimidin-4-yl]-3,8-diazabicyclo[3.2.1]octane-8-carboxylate and [(3R,8R)-8-[[tert-butyl(diphenyl)silyl]oxymethyl]-1,2,3,5,6,7-hexahydropyrrolizin-3-yl]methanol. (formic acid salt, white solid). LC/MS (ESI) m/z: 1192.0. [M+H]⁺. ¹H NMR (400 MHz, CD₃OD) δ 9.16-9.13 (m, 1H), 8.88 (s, 1H), 7.62 (d, J=8.0 Hz, 1H), 7.46-7.28 (m, 6H), 7.16 (d, J=8.0 Hz, 1H), 7.03-7.01 (m, 1H), 6.12-6.08 (m, 1H), 5.05-5.02 (m, 1H), 4.78-4.57 (m, 5H), 4.51-4.47 (m, 1H), 4.41-4.32 (m, 2H), 4.25-4.18 (m, 1H), 4.12-4.00 (m, 1H), 3.92-3.74 (m, 5H), 3.67-3.55 (m, 7H), 3.23-3.13 (m, 3H), 2.48-2.45 (m, 3H), 2.38-1.85 (m, 18H), 1.55-1.46 (m, 3H), 1.06-1.02 (m, 3H), 0.92-0.63 (m, 6H).

Exemplary Synthesis of [(3R,8S)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-thiazol-5-yl)phenyl]ethyl]carbamoyl]pyrrolidine-1-carbonyl]-2-methyl-methyl propyl]isoxazol-3-yl]piperazine-1-carboxylate (Compound 330)

Step 1: Preparation of tert-butyl 3-(7-bromo-2-chloro-8-fluoro-quinazolin-4-yl)-3,8-diazabicyclo[3.2.1]octane-8-carboxylate

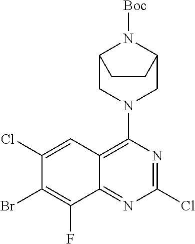 [SMILES: CC(C)(C)OC(=O)N1C2CCC1CN(c1nc(Cl)nc3c(F)c(Br)c(Cl)cc13)C2]

To a mixture of 7-bromo-2,4-dichloro-8-fluoro-quinazoline (10 g, 33.79 mmol, 1 eq) and tert-butyl 3,8-diazabicyclo[3.2.1]octane-8-carboxylate (7.17 g, 33.79 mmol, 1 eq) in dioxane (200 mL) was added diisopropylethylamine (21.84 g, 168.96 mmol, 29 mL, 5 eq) in one portion at 25° C. under nitrogen, and the reaction mixture was stirred at 25° C. for 1 h. The mixture was diluted with water (400 mL) and stirred at 25° C. for 30 minutes. The precipitated solid was collected by filtration, washed with water (100 mL) followed by ethyl acetate (50 mL), then dried under vacuum to afford tert-butyl 3-(7-bromo-2-chloro-8-fluoro-quinazolin-4-yl)-3,8-diazabicyclo[3.2.1]octane-8-carboxylate (13.6 g, 28.83 mmol, 85% yield) as a yellow solid.

Step 2: Preparation of tert-butyl 3-(7-bromo-2,8-difluoro-quinazolin-4-yl)-3,8-diazabicyclo[3.2.1]octane-8-carboxylate

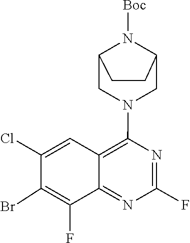 [SMILES: CC(C)(C)OC(=O)N1C2CCC1CN(c1nc(F)nc3c(F)c(Br)c(Cl)cc13)C2]

To a mixture of tert-butyl 3-(7-bromo-2-chloro-8-fluoro-quinazolin-4-yl)-3,8-diazabicyclo[3.2.1]octane-8-carboxylate (13.6 g, 28.83 mmol, 1 eq) in N,N-dimethylacetamide (140 mL) was added potassium fluoride (83.74 g, 1.44 mol, 50 eq) in one portion at 25° C. under nitrogen, and the reaction mixture was stirred at for 6 hours. The mixture was cooled to 25° C., diluted with water (200 mL), and stirred for 15 minutes. The aqueous phase was extracted with ethyl acetate (300 mL×3), and the combined organic extract was washed with brine (300 mL×3), dried with anhydrous sodium sulfate, filtered, and concentrated. The residue was purified by silica gel chromatography (Petroleum ether/Ethyl acetate=20/1, 10/1) to afford tert-butyl 3-(7-bromo-2,8-difluoro-quinazolin-4-yl)-3,8-diazabicyclo[3.2.1]octane-8-carboxylate (8.2 g, 18.01 mmol, 62% yield) as a yellow solid. ¹HNMR (400 MHz, DMSO-d₆) δ 8.14 (s, 1H), 4.38 (d, J=12.0 Hz, 1H), 4.25 (s, 2H), 3.65 (d, J=7.2 Hz, 2H), 1.77 (s, 2H), 1.76-1.63 (m, 2H), 1.61 (s, 9H).

Step 3: Preparation of tert-butyl 3-[7-bromo-2-[[(3S,8R)-3-[[tert-butyl(diphenyl)silyl]oxymethyl]-1,2,3,5,6,7-hexahydropyrrolizin-8-yl]methoxy]-6-chloro-8-fluoro-quinazolin-4-yl]-3,8-diazabicyclo[3.2.1]octane-8-carboxylate

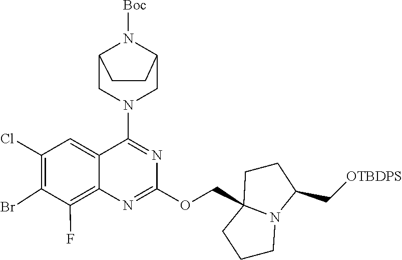

To solution of tert-butyl 3-(7-bromo-6-chloro-2,8-difluoro-quinazolin-4-yl)-3,8-diazabicyclo[3.2.1]octane-8-carboxylate (1 g, 2.04 mmol, 1 eq) and [(3S,8R)-3-[[tert-butyl(diphenyl)silyl]oxymethyl]-1,2,3,5,6,7-hexahydropyrrolizin-8-yl]methanol (1.10 g, 2.69 mmol, 1.32 eq) in tetrahydrofuran (20 mL) at 0° C. was added lithium tert-butoxide (2.2 M, 2.8 mL, 3.0 eq) dropwise, and the reaction mixture was stirred for 12 hours at 25° C. under nitrogen. The reaction was quenched with saturated aqueous ammonium chloride (20 mL), then extracted with ethyl acetate (20 mL×3). The combined organic extract was washed with brine (20 mL), dried over anhydrous sodium sulfate, filtered, and concentrated. The residue was purified by silica gel chromatography (Petroleum ether/Ethyl acetate=from 10/1 to 0/1) to afford tert-butyl 3-[7-bromo-2-[[(3S,8R)-3-[[tert-butyl(diphenyl)silyl]oxymethyl]-1,2,3,5,6,7-hexahydropyrrolizin-8-yl]methoxy]-6-chloro-8-fluoro-quinazolin-4-yl]-3,8-diazabicyclo[3.2.1]octane-8-carboxylate (1.1 g, 1.25 mmol, 61% yield) as a white solid.

Step 4: Preparation of tert-butyl 3-[2-[[(3R,8S)-3-[[tert-butyl(diphenyl)silyl]oxymethyl]-1,2,3,5,6,7-hexahydropyrrolizin-8-yl]methoxy]-6-chloro-8-fluoro-7-[3-(methoxymethoxy)-1-naphthyl]quinazolin-4-yl]-3,8-diazabicyclo[3.2.1]octane-8-carboxylate

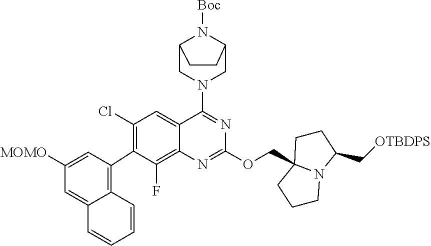

A solution of 2-[3-(methoxymethoxy)-1-naphthyl]-4,4,5,5-tetramethyl-1,3,2-dioxaborolane (472 mg, 1.50 mmol, 1.2 eq), tert-butyl 3-[7-bromo-2-[[(3S,8R)-3-[[tert-butyl(diphenyl)silyl]oxymethyl]-1,2,3,5,6,7-hexahydropyrrolizin-8-yl]methoxy]-6-chloro-8-fluoro-quinazolin-4-yl]-3,8-diazabicyclo[3.2.1]octane-8-carboxylate (1.10 g, 1.25 mmol, 1 eq), dicyclohexyl-[2-(2,6-dimethoxyphenyl)phenyl]phosphane; methanesulfonate; (2-phenylanilino) palladium (1+) (98 mg, 0.13 mmol, 0.1 eq), and potassium phosphate (1.5 M, 2.5 mL, 3 eq) in tetrahydrofuran (20 mL) was stirred for 3 h at 60° C. under nitrogen. The mixture was diluted with water (10 mL) and extracted with ethyl acetate (10 mL×3). The combined organic extract was washed with brine (10 mL), dried over anhydrous sodium sulfate, filtered, and concentrated. The residue was purified by prep-HPLC {column: Phenomenex luna C18 (250*70 mm, 10 um); mobile phase: [50-80% CH₃CN in water (TFA)]} to afford tert-butyl 3-[2-[[(3S,8R)-3-[[tert-butyl(diphenyl)silyl]oxymethyl]-1,2,3,5,6,7-hexahydropyrrolizin-8-yl]methoxy]-6-chloro-8-fluoro-7-[3-(methoxymethoxy)-1-naphthyl]quinazolin-4-yl]-3,8-diazabicyclo[3.2.1]octane-8-carboxylate (800 mg, 0.81 mmol, 64% yield, TFA salt) as a yellow solid. LC/MS (ESI) m/z: 986.4 [M+H]⁺. ¹H NMR (400 MHz, CDCl₃) δ: 7.84 (d, J=8.4 Hz, 1H), 7.78-7.65 (m, 6H), 7.56-7.16 (m, 2H), 7.51-7.36 (m, 6H), 7.24-7.11 (m, 2H), 5.38-5.33 (m, 2H), 4.48-4.28 (m, 4H), 3.73-3.65 (m, 1H), 3.56 (s, 3H), 3.05-2.80 (m, 2H), 2.74-2.60 (m, 1H), 2.35-2.18 (m, 1H), 1.98-1.93 (m, 2H), 1.88-1.78 (m, 4H), 1.72-1.58 (m, 6H), 1.54 (s, 9H), 1.50-1.43 (m, 4H), 1.05 (s, 9H).

Step 5: Preparation of tert-butyl 3-[6-chloro-8-fluoro-2-[[(3R,8S)-3-(hydroxymethyl)-1,2,3,5,6,7-hexahydropyrrolizin-8-yl]methoxy]-7-[3-(methoxymethoxy)-1-naphthyl]quinazolin-4-yl]-3,8-diazabicyclo[3.2.1]octane-8-carboxylate

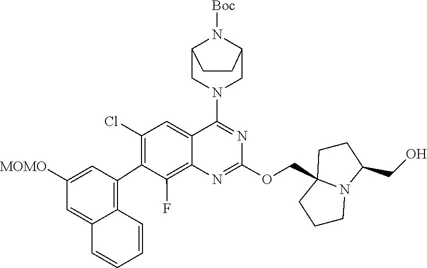

To a solution of tert-butyl 3-[2-[[(3S,8R)-3-[[tert-butyl(diphenyl)silyl]oxymethyl]-1,2,3,5,6,7-hexahydropyrrolizin-8-yl]methoxy]-6-chloro-8-fluoro-7-[3-(methoxymethoxy)-1-naphthyl]quinazolin-4-yl]-3,8-diazabicyclo[3.2.1]octane-8-carboxylate (800 mg, 0.81 mmol, 1 eq) in DMF (10 mL) was added cesium fluoride (2.46 g, 16.22 mmol, 20 eq), and the reaction mixture was stirred for 12 h at 25° C. under nitrogen. The reaction mixture was diluted with water (20 mL) extracted with ethyl acetate (20 mL×3). The combined organic extract was washed with brine (20 mL), dried over anhydrous sodium sulfate, filtered, and concentrated. The residue was purified by preparative TLC (Dichloromethane/Methanol=10/1) to afford tert-butyl 3-[6-chloro-8-fluoro-2-[[(3R,8S)-3-(hydroxylmethyl)-1,2,3,5,6,7-hexahydropyrrolizin-8-yl]methoxy]-7-[3-(methoxymethoxy)-1-naphthyl]quinazolin-4-yl]-3,8-diazabicyclo[3.2.1]octane-8-carboxylate (440 mg, 0.59 mmol, 72% yield) as a yellow solid. LC/MS (ESI) m/z: 748.4 [M+H]⁺.

Step 6: Preparation of tert-butyl 3-[6-chloro-8-fluoro-2-[[(3R,8S)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]-7-[3-(methoxymethoxy)-1-naphthyl]quinazolin-4-yl]-3,8-diazabicyclo[3.2.1]octane-8-carboxylate

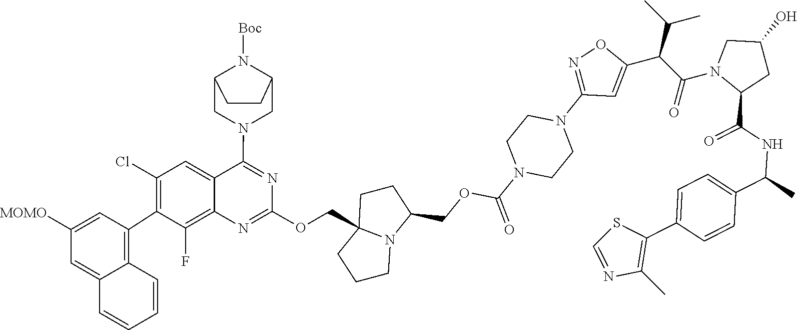

To a solution of tert-butyl 3-[6-chloro-8-fluoro-2-[[(3S,8R)-3-(hydroxymethyl)-1,2,3,5,6,7-hexahydropyrrolizin-8-yl]methoxy]-7-[3-(methoxymethoxy)-1-naphthyl]quinazolin-4-yl]-3,8-diazabicyclo[3.2.1]octane-8-carboxylate (240 mg, 0.32 mmol, 1 eq) and (4-nitrophenyl) carbonochloridate (110 mg, 0.55 mmol, 1.7 eq) in tetrahydrofuran (5 mL) were added triethylamine (162 mg, 1.60 mmol, 5 eq) and dimethyl aminopyridine (4 mg, 0.03 mmol, 0.1 eq), and the reaction mixture was stirred for 12 h at 25° C. under nitrogen. A solution of (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide (216 mg, 0.38 mmol, 1.2 eq) in tetrahydrofuran (5 mL) was then added, and the reaction mixture was stirred at 25° C. for 2 hour. The residue was purified by prep-HPLC {column: Phenomenex Luna C18 150*25 mm*10 um; mobile phase: [33-63% CH₃CN in water (formic acid)]} to afford tert-butyl 3-[6-chloro-8-fluoro-2-[[(3R,8S)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]-7-[3-(methoxymethoxy)-1-naphthyl]quinazolin-4-yl]-3,8-diazabicyclo[3.2.1]octane-8-carboxylate (150 mg, 0.11 mmol, 35% yield, formic acid salt) as a white solid. LC/MS (ESI) m/z: 1340.9 [M+H]⁺.

Step 7: Preparation of [(3R,8S)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]piperazine-1-carboxylate

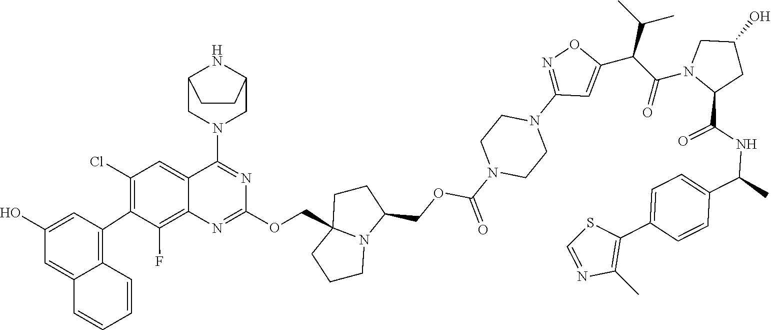 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@@]5(COc6nc(N7CC8CCC(C7)N8)c7cc(Cl)c(-c8cc(O)cc9ccccc89)c(F)c7n6)CCCN45)CC3)no2)C(C)C)cc1]

A solution of tert-butyl 3-[6-chloro-8-fluoro-2-[[(3S,8R)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1,2,3,5,6,7-hexahydropyrrolizin-8-yl]methoxy]-7-[3-(methoxymethoxy)-1-naphthyl]quinazolin-4-yl]-3,8-diazabicyclo[3.2.1]octane-8-carboxylate (130 mg, 0.09 mmol, 1 eq) in formic acid (5 mL) was stirred for 2 h at 25° C. The reaction mixture was concentrated, and the resulting residue was purified by prep-HPLC {column: Phenomenex luna C18 150*40 mm*15 um; mobile phase: [15-45% CH₃CN in water (formic acid)]} to afford [(3R,8S)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (48.1 mg, 0.04 mmol, 38% yield, formic acid salt) as a white solid. LC/MS (ESI) m/z: 1196.4 [M+H]⁺. ¹H NMR (400 MHz, DMSO-d₆) δ 8.98 (s, 1H), 8.40 (d, J=7.6 Hz, 1H), 8.19 (s, 2H), 7.92 (s, 1H), 7.80 (d, J=8.4 Hz, 1H), 7.46-7.41 (m, 3H), 7.38-7.35 (m, 2H), 7.30-7.26 (m, 1H), 7.23-7.19 (m, 2H), 7.08-7.04 (m, 1H), 6.17-6.05 (m, 1H), 4.97-4.86 (m, 1H), 4.40-4.25 (m, 5H), 4.10-4.00 (m, 3H), 3.95-3.80 (m, 4H), 3.72-3.66 (m, 3H), 3.17-3.10 (m, 6H), 2.97-2.87 (m, 3H), 2.45 (s, 3H), 2.25-2.15 (m, 2H), 2.10-1.90 (m, 4H), 1.85-1.75 (m, 4H), 1.73-1.65 (m, 5H), 1.64-1.51 (m, 3H), 1.45-1.35 (m, 4H), 0.97-0.92 (m, 3H), 0.83-0.75 (m, 3H).

Exemplary Synthesis of [(3R,8R)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]piperazine-1-carboxylate (Compound 329)

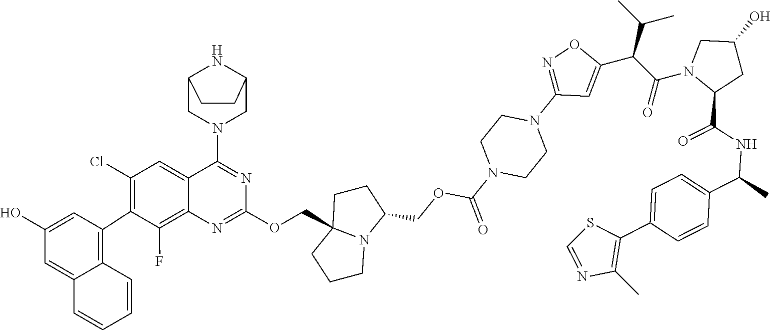 [SMILES: Cc1ncsc1-c1ccc([C@H](C)NC(=O)[C@@H]2C[C@@H](O)CN2C(=O)[C@@H](c2cc(N3CCN(C(=O)OC[C@H]4CC[C@@]5(COc6nc(N7CC8CCC(C7)N8)c7cc(Cl)c(-c8cc(O)cc9ccccc89)c(F)c7n6)CCCN45)CC3)no2)C(C)C)cc1]

The title compound was made in an analogous manner to [(3R,8S)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-(7-bromo-6-chloro-2,8-difluoro-quinazolin-4-yl)-3,8-diazabicyclo[3.2.1]octane-8-carboxylate and [(3R,8R)-3-[[tert-butyl(diphenyl)silyl]oxymethyl]-1,2,3,5,6,7-hexahydropyrrolizin-8-yl]methanol. (formic acid salt, white solid). LC/MS (ESI) m/z: 1196.7 [M+H]⁺. ¹H NMR (400 MHz, DMSO-d₆) δ: 8.98 (s, 1H), 8.40 (d, J=7.6 Hz, 1H), 8.21 (s, 2H), 7.93 (s, 1H), 7.80 (d, J=8.4 Hz, 1H), 7.47-7.41 (m, 3H), 7.38-7.35 (m, 2H), 7.29-7.26 (m, 1H), 7.23-7.19 (m, 2H), 7.08-7.04 (m, 1H), 6.18-6.02 (m, 1H), 4.96-4.84 (m, 1H), 4.39-4.32 (m, 3H), 4.29-4.23 (m, 2H), 4.20-4.11 (m, 3H), 4.08-4.01 (m, 2H), 3.73-3.71 (m, 2H), 3.61-3.57 (m, 6H), 3.33-3.27 (m, 3H), 3.19-3.12 (m, 5H), 2.83-2.70 (m, 2H), 2.45 (s, 3H), 2.25-2.14 (m, 2H), 2.09-1.98 (m, 2H), 1.80-1.69 (m, 10H), 1.56-1.48 (m, 1H), 1.44-1.34 (m, 3H), 0.97-0.90 (m, 3H), 0.82-0.75 (m, 3H).

Exemplary Synthesis of [(3S,8S)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]piperazine-1-carboxylate (Compound 328)

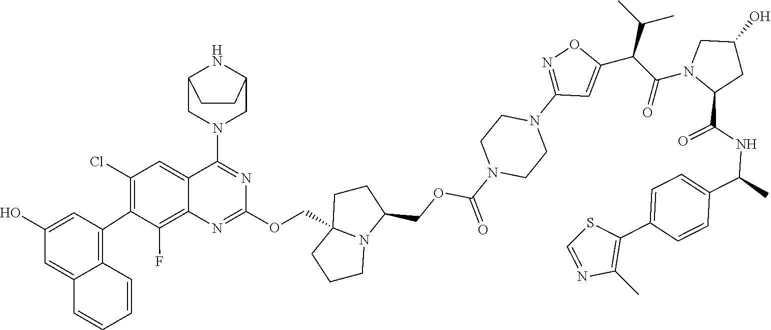 [SMILES: Cc1ncsc1-c1ccc([C@H](C)NC(=O)[C@@H]2C[C@@H](O)CN2C(=O)[C@@H](c2cc(N3CCN(C(=O)OC[C@@H]4CC[C@]5(COc6nc(N7CC8CCC(C7)N8)c7cc(Cl)c(-c8cc(O)cc9ccccc89)c(F)c7n6)CCCN45)CC3)no2)C(C)C)cc1]

The title compound was made in an analogous manner to [(3R,8S)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-(7-bromo-6-chloro-2,8-difluoro-quinazolin-4-yl)-3,8-diazabicyclo[3.2.1]octane-8-carboxylate (1 g, 2.04 mmol, 1 eq) and [(3S,8S)-3-[[tert-butyl(diphenyl)silyl]oxymethyl]-1,2,3,5,6,7-hexahydropyrrolizin-8-yl]methanol. (formic acid salt, white solid). LC/MS (ESI) m/z: 1196.7 [M+H]⁺. ¹H NMR (400 MHz, DMSO-d₆) δ 8.98 (s, 1H), 8.40 (d, J=7.6 Hz, 1H), 8.21 (s, 2H), 7.93 (s, 1H), 7.80 (d, J=8.4 Hz, 1H), 7.47-7.40 (m, 3H), 7.38-7.33 (m, 2H), 7.29-7.25 (m, 1H), 7.22-7.19 (m, 2H), 7.07-7.02 (m, 1H), 6.17-6.05 (m, 1H), 4.95-4.84 (m, 1H), 4.39-4.32 (m, 3H), 4.30-4.25 (m, 2H), 4.20-4.11 (m, 3H), 4.08-4.01 (m, 2H), 3.72-3.68 (m, 3H), 3.60-3.57 (m, 6H), 3.33-3.27 (m, 4H), 3.19-3.12 (m, 5H), 2.82-2.70 (m, 2H), 2.45 (s, 3H), 2.25-2.18 (m, 1H), 2.09-1.98 (m, 2H), 1.83-1.69 (m, 10H), 1.56-1.48 (m, 1H), 1.46-1.35 (m, 3H), 0.98-0.91 (m, 3H), 0.83-0.74 (m, 3H).

Exemplary Synthesis of [(3S,8R)-8-[[4-(3,8-diazabicyclo[3.2.1]octan-3-yl)-7-(8-ethyl-3-hydroxy-1-naphthyl)-8-fluoro-quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 267)

Step 1: Preparation of methyl 2-amino-4-bromo-3-fluoro-benzoate

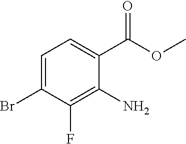

To a solution of 2-amino-4-bromo-3-fluoro-benzoic acid (25 g, 106.83 mmol, 1 eq) in methanol (250 mL) was added sulfuric acid (52.39 g, 534.14 mmol, 28.5 mL, 5 eq), and the reaction mixture was stirred at 60° C. for 12 h. The reaction mixture was diluted with water (500 mL) and extracted with ethyl acetate (200 mL×3). The combined organic extract was washed with sodium hydrogen carbonate (200 mL×2) and brine (500 mL), dried over anhydrous sodium sulfate, filtered, and concentrated. The crude product was triturated with petroleum ether (100 ml) at 25° C. for 60 minutes to afford methyl 2-amino-4-bromo-3-fluoro-benzoate (19.2 g, 77.40 mmol, 72% yield) as a white solid. ¹H NMR (400 MHz, CDCl₃) δ 7.52 (dd, J=1.6, 8.8 Hz, 1H), 6.77 (dd, J=6.4, 8.8 Hz, 1H), 5.92 (s, 2H), 3.89 (s, 3H).

Step 2: Preparation of methyl 4-bromo-3-fluoro-2-[(2,2,2-trichloroacetyl) carbamoylamino]benzoate

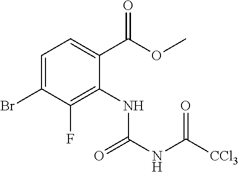

To a solution of methyl 2-amino-4-bromo-3-fluoro-benzoate (500 mg, 2.02 mmol, 1 eq) in tetrahydrofuran (5 mL) at 0° C. was added a solution of 2,2,2-trichloroacetyl isocyanate (570 mg, 3.02 mmol, 0.4 mL, 1.5 eq) in tetrahydrofuran (5 mL) dropwise, and the reaction mixture was stirred at 25° C. for 1 h. The mixture was concentrated, and the crude product was triturated with methyl tert-butyl ether (100 ml) at 25° C. for 30 minutes to afford methyl 4-bromo-3-fluoro-2-[(2,2,2-trichloroacetyl)carbamoylamino]benzoate (819 mg, 1.88 mmol, 93% yield) as a white solid. ¹H NMR (400 MHz, CDCl₃) δ: 10.64 (s, 1H), 8.66 (s, 1H), 7.70 (dd, J=1.6, 8.8 Hz, 1H), 7.56 (dd, J=6.0, 8.8 Hz, 1H), 3.96 (s, 3H).

Step 3: Preparation of 7-bromo-8-fluoro-1H-quinazoline-2,4-dione

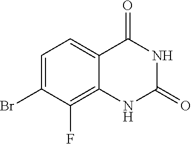 [SMILES: O=c1[nH]c(=O)c2ccc(Br)c(F)c2[nH]1]

To a solution of methyl 4-bromo-3-fluoro-2-[(2,2,2-trichloroacetyl)carbamoylamino]benzoate (819 mg, 1.88 mmol, 1 eq) in methanol (7 mL) 0° C. was added ammonia (7 M, 0.8 mL, 3 eq) in methanol (1 mL), and the reaction mixture was stirred at 25° C. for 1 h. The mixture was concentrated, and the crude product was triturated with methyl tert-butyl ether (20 ml) at 25° C. for 30 minutes to afford 7-bromo-8-fluoro-1H-quinazoline-2,4-dione (392 mg, 1.51 mmol, 81% yield) as a white solid. ¹H NMR (400 MHz, DMSO-d₆) δ 11.60-11.39 (m, 2H), 7.63 (dd, J=1.2, 8.8 Hz, 1H), 7.44 (dd, J=6.0, 8.8 Hz, 1H).

Step 4: Preparation of 7-bromo-2,4-dichloro-8-fluoro-quinazoline

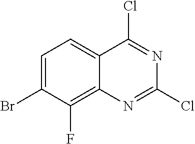 [SMILES: Fc1c(Br)ccc2c(Cl)nc(Cl)nc12]

To a solution of 7-bromo-8-fluoro-1H-quinazoline-2,4-dione (6 g, 23.16 mmol, 1 eq) in phosphoryl chloride (60 mL) at 0° C. was added diisopropylethylamine (5.99 g, 46.33 mmol, 8.1 mL, 2 eq), and the reaction mixture was stirred at 90° C. for 2 h. The mixture was concentrated under vacuum, to afford 7-bromo-2,4-dichloro-8-fluoro-quinazoline (6.8 g, crude) as a yellow solid.

Step 5: Preparation of tert-butyl 3-(7-bromo-2-chloro-8-fluoro-quinazolin-4-yl)-3,8-diazabicyclo[3.2.1]octane-8-carboxylate

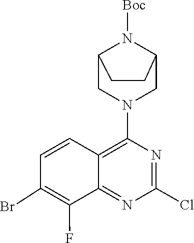 [SMILES: CC(C)(C)OC(=O)N1C2CCC1CN(c1nc(Cl)nc3c(F)c(Br)ccc13)C2]

To a solution of 7-bromo-2,4-dichloro-8-fluoro-quinazoline (6.8 g, 22.98 mmol, 1 eq) in dichloromethane (60 mL) at −60° C. were added tert-butyl(1S,5R)-3,8-diazabicyclo[3.2.1]octane-8-carboxylate (4.39 g, 20.68 mmol, 0.9 eq) and diisopropylethylamine (14.85 g, 114.89 mmol, 20 mL, 5 eq), and the reaction mixture was stirred at −60° C. for 1 h. The reaction mixture was returned to room temperature, diluted with water (50 mL), and extracted with dichloromethane (15 mL×3). The combined organic extract was washed with brine (50 mL), dried over anhydrous sodium sulfate, filtered, and concentrated in vacuum. The residue was purified by column chromatography (silicon dioxide, Petroleum ether/Ethyl acetate=9/1 to 4/1) to afford tert-butyl 3-(7-bromo-2-chloro-8-fluoro-quinazolin-4-yl)-3,8-diazabicyclo[3.2.1]octane-8-carboxylate (1.8 g, 3.82 mmol, 17% yield) as a yellow solid. LC/MS (ESI) m/z: 471.1 [M+H]⁺. ¹H NMR (400 MHz, CDCl₃) δ 7.52-7.48 (m, 2H), 4.51-4.28 (m, 4H), 3.75-3.53 (m, 2H), 2.00-1.91 (m, 2H), 1.74 (s, 2H), 1.52 (s, 9H).

Step 6: Preparation of tert-butyl 3-(7-bromo-2,8-difluoro-quinazolin-4-yl)-3,8-diazabicyclo[3.2.1]octane-8-carboxylate

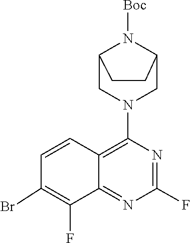 [SMILES: CC(C)(C)OC(=O)N1C2CCC1CN(c1nc(F)nc3c(F)c(Br)ccc13)C2]

To a solution of tert-butyl 3-(7-bromo-2-chloro-8-fluoro-quinazolin-4-yl)-3,8-diazabicyclo[3.2.1]octane-8-carboxylate (3.5 g, 7.42 mmol, 1 eq) in N,N-dimethylacetamide (50 mL) was added potassium fluoride (21.55 g, 370.96 mmol, 8.7 mL, 50 eq), and the reaction mixture was stirred at 120° C. for 8 h. The reaction was cooled to room temperature, diluted with ethyl acetate (1.5 L), and washed with water (3×500 mL). The organic layer was dried over anhydrous sodium sulfate and concentrated. The residue was purified by flash silica gel chromatography (gradient: 0˜25% ethyl acetate in petroleum ether) to afford tert-butyl 3-(7-bromo-2,8-difluoro-quinazolin-4-yl)-3,8-diazabicyclo[3.2.1]octane-8-carboxylate (1.4 g, 3.07 mmol, 41% yield) as yellow solid. LC/MS (ESI) m/z: 457.1 [M+H]⁺.

Step 7: Preparation of tert-butyl 3-[7-bromo-2-[[(3S,8R)-3-[[tert-butyl(diphenyl)silyl]oxymethyl]-1,2,3,5,6,7-hexahydropyrrolizin-8-yl]methoxy]-8-fluoro-quinazolin-4-yl]-3,8-diazabicyclo[3.2.1]octane-8-carboxylate

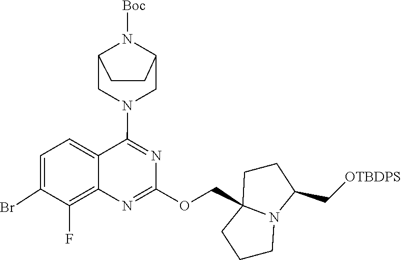

To a solution of tert-butyl 3-(7-bromo-2,8-difluoro-quinazolin-4-yl)-3,8-diazabicyclo[3.2.1]octane-8-carboxylate (1 g, 2.20 mmol, 1 eq) and [(3S,8R)-3-[[tert-butyl(diphenyl) silyl]oxymethyl]-1,2,3,5,6,7-hexahydropyrrolizin-8-yl]methanol (720 mg, 1.76 mmol, 0.8 eq) in tetrahydrofuran (10 mL) at 0° C. purged with nitrogen (3×) was added lithium tert-butoxide (2.2 M, 3 mL, 3 eq), and the reaction mixture was stirred at 25° C. for 12 h. The reaction mixture was diluted with water (200 mL) and extracted with ethyl acetate (50 mL×3). The combined organic extract was washed with brine (200 mL), dried over anhydrous sodium sulfate, filtered, and concentrated. The residue was purified by flash silica gel chromatography (gradient: 0˜40% ethyl acetate in petroleum ether) to afford tert-butyl 3-[7-bromo-2-[[(3S,8R)-3-[[tert-butyl(diphenyl)silyl]oxymethyl]-1,2,3,5,6,7-hexahydropyrrolizin-8-yl]methoxy]-8-fluoro-quinazolin-4-yl]-3,8-diazabicyclo[3.2.1]octane-8-carboxylate (570 mg, 0.67 mmol, 31% yield) as a white solid. LC/MS (ESI) m/z: 846.4 [M+H]⁺. ¹H NMR (400 MHz, CDCl₃) δ 7.77-7.68 (m, 4H), 7.46-7.34 (m, 8H), 4.41-4.21 (m, 4H), 4.11-4.07 (m, 2H), 3.70 (dd, J=5.6, 9.6 Hz, 1H), 3.56 (dd, J=6.8, 10.0 Hz, 2H), 2.99 (td, J=6.0, 10.8 Hz, 1H), 2.91-2.80 (m, 1H), 2.73-2.61 (m, 1H), 2.23 (dd, J=4.8, 12.8 Hz, 1H), 2.03-1.93 (m, 2H), 1.92-1.86 (m, 2H), 1.86-1.79 (m, 2H), 1.77-1.65 (m, 4H), 1.57-1.53 (m, 2H), 1.52 (s, 9H), 1.06 (s, 9H).

Step 8: Preparation of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxy methoxy)-1-naphthyl]-8-fluoro-quinazolin-4-yl]-3,8-diazabicyclo[3.2.1]octane-8-carboxylate

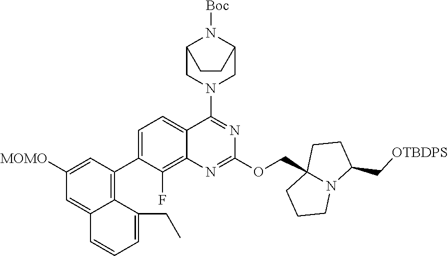

To a mixture of tert-butyl 3-[7-bromo-2-[[(3S,8R)-3-[[tert-butyl(diphenyl)silyl]oxymethyl]-1,2,3,5,6,7-hexahydropyrrolizin-8-yl]methoxy]-8-fluoro-quinazolin-4-yl]-3,8-diazabicyclo[3.2.1]octane-8-carboxylate (570 mg, 0.67 mmol, 1 eq), 2-[8-ethyl-3-(methoxymethoxy)-1-naphthyl]-4,4,5,5-tetramethyl-1,3,2-dioxaborolane (277 mg, 0.81 mmol, 1.2 eq), and potassium phosphate (430 mg, 2.02 mmol, 3 eq) in tetrahydrofuran (8 mL) was added dicyclohexyl-[2-(2,6-dimethoxyphenyl)phenyl]phosphane; methanesulfonate; (2-phenylanilino) palladium (1+) (53 mg, 0.07 mmol, 0.1 eq), and the resulting mixture was degassed and purged with nitrogen (3×), then stirred at 60° C. for 12 h under nitrogen atmosphere. The reaction mixture was diluted with water (50 mL) and extracted with ethyl acetate (15 mL×3). The combined organic extract was washed with brine (50 mL), dried over anhydrous sodium sulfate, filtered, and concentrated. The residue was purified by flash silica gel chromatography (gradient: 0˜40% ethyl acetate in petroleum ether) to afford tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-quinazolin-4-yl]-3,8-diazabicyclo[3.2.1]octane-8-carboxylate (448 mg, 0.046 mmol, 68% yield) as a yellow oil. LC/MS (ESI) m/z: 980.7 [M+H]⁺. ¹H NMR (400 MHz, CDCl₃) δ 7.75-7.67 (m, 4H), 7.56 (d, J=8.8 Hz, 1H), 7.49 (d, J=2.4 Hz, 1H), 7.45-7.33 (m, 7H), 7.24-7.19 (m, 1H), 7.06 (s, 2H), 5.31 (s, 2H), 4.49-4.22 (m, 4H), 4.18-4.05 (m, 2H), 3.74-3.68 (m, 1H), 3.57 (d, J=6.8 Hz, 1H), 3.53 (s, 3H), 3.05-2.80 (m, 1H), 2.71-2.37 (m, 2H), 2.31-2.21 (m, 1H), 2.09-1.91 (m, 4H), 1.89-1.79 (m, 3H), 1.75-1.63 (m, 2H), 1.56 (s, 1H), 1.53 (s, 9H), 1.28-1.23 (m, 2H), 1.06 (s, 8H), 0.91 (t, J=7.2 Hz, 3H).

Step 9: Preparation of tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]quinazolin-4-yl]-3,8-diazabicyclo[3.2.1]octane-8-carboxylate

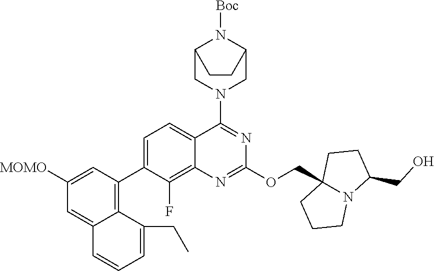

To a solution of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]-8-fluoro-quinazolin-4-yl]-3,8-diazabicyclo[3.2.1]octane-8-carboxylate (448 mg, 0.46 mmol, 1 eq) in DMF (5 mL) was added cesium fluoride (1.04 g, 6.86 mmol, 15 eq), and the reaction mixture was stirred at 25° C. for 12 h. The reaction mixture was filtered, and the filtrate was diluted with water (100 mL) and extracted with ethyl acetate (20 mL×3). The combined organic extract was washed with brine (100 mL), dried over anhydrous sodium sulfate, filtered, and concentrated. The residue was purified by prep-TLC (SiO₂, dichloromethane:methanol=8:1) to afford tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]quinazolin-4-yl]-3,8-diazabicyclo[3.2.1]octane-8-carboxylate (275 mg, 0.37 mmol, 81% yield) as a yellow solid. LC/MS (ESI) m/z: 742.4 [M+H]⁺.

Step 10: Preparation of [(3S,8R)-8-[[4-(3,8-diazabicyclo[3.2.1]octan-3-yl)-7-(8-ethyl-3-hydroxy-1-naphthyl)-8-fluoro-quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

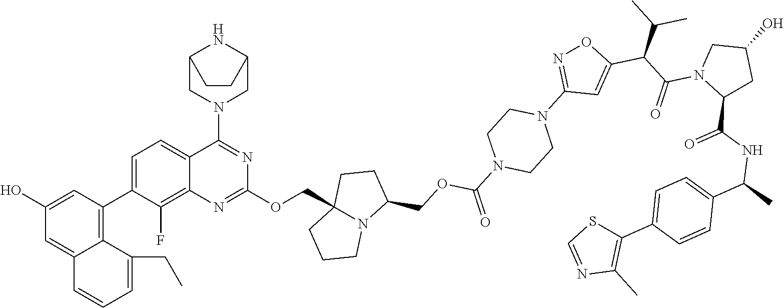 [SMILES: CCc1cccc2cc(O)cc(-c3ccc4c(N5CC6CCC(C5)N6)nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was made in an analogous manner to [(3R,8S)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-8-fluoro-2-[[(3S,8R)-3-(hydroxymethyl)-1,2,3,5,6,7-hexahydropyrrolizin-8-yl]methoxy]quinazolin-4-yl]-3,8-diazabicyclo[3.2.1]octane-8-carboxylate and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid salt, yellow solid). LC/MS (ESI) m/z: 1191.8 [M+H]⁺. ¹H NMR (400 MHz, DMSO-d₆) δ 8.98 (s, 1H), 8.43-8.35 (m, 1H), 8.20 (s, 1H), 7.83-7.75 (m, 1H), 7.68-7.62 (m, 1H), 7.46-7.41 (m, 2H), 7.38-7.32 (m, 3H), 7.26-7.18 (m, 2H), 7.11 (d, J=6.8 Hz, 1H), 6.88 (d, J=2.8 Hz, 1H), 6.18-6.03 (m, 1H), 4.95-4.86 (m, 1H), 4.40-4.22 (m, 4H), 4.10-3.85 (m, 5H), 3.74 (d, J=6.4 Hz, 2H), 3.58 (dd, J=5.2, 15.2 Hz, 6H), 3.15 (s, 9H), 2.99-2.88 (m, 4H), 2.45 (s, 3H), 2.27-2.12 (m, 3H), 2.09-1.90 (m, 4H), 1.80 (d, J=4.8 Hz, 3H), 1.72 (s, 3H), 1.63-1.59 (m, 1H), 1.44 (d, J=7.2 Hz, 1H), 1.40-1.33 (m, 3H), 1.26-1.21 (m, 1H), 0.98-0.92 (m, 3H), 0.82-0.76 (m, 6H).

Exemplary Synthesis of [(3S,8R)-8-[[4-(3,8-diazabicyclo[3.2.1]octan-3-yl)-7-(8-ethyl-3-hydroxy-1-naphthyl)pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (Compound 266)

Step 1: Preparation of 4-amino-6-chloro-pyridine-3-carbonyl chloride 7-chloro-2-thioxo-1H-pyrido[4,3-d]pyrimidin-4-one

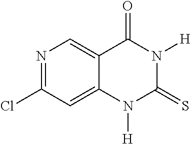 [SMILES: O=c1[nH]c(=S)[nH]c2cc(Cl)ncc12]

To a solution 4-amino-6-chloro-pyridine-3-carboxylic acid (20 g, 115.90 mmol, 1 eq) in phosphoryl chloride (200 mL) was stirred at 90° C. for 2 h. The mixture was concentrated under vacuum. To the resulting residue in tetrahydrofuran (200 mL) was added ammonium thiocyanate (17.53 g, 230.35 mmol, 18 mL, 2 eq) in tetrahydrofuran (200 mL), and the reaction mixture was stirred at 60° C. for 12 h. The mixture was filtered, and the filtrate was concentrated. The residue was triturated with ethyl acetate (300 mL) to afford 7-chloro-2-thioxo-1H-pyrido[4,3-d]pyrimidin-4-one (17 g, 79.57 mmol, 69% yield) as a yellow solid. ¹H NMR (400 MHz, DMSO-d₆) δ 12.93 (s, 1H), 12.79 (s, 1H), 8.80 (s, 1H), 7.20 (s, 1H).

Step 2: Preparation of 7-chloro-2-methylsulfanyl-3H-pyrido[4,3-d]pyrimidin-4-one

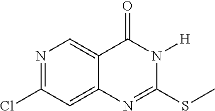

To a solution of 7-chloro-2-thioxo-1H-pyrido[4,3-d]pyrimidin-4-one (17 g, 79.57 mmol, 1 eq) in DMF (170 mL) at 25° C. was added sodium methoxide (4.30 g, 79.57 mmol, 1 eq) for 10 minutes followed by iodomethane (11.29 g, 79.57 mmol, 5 mL, 1 eq), and the reaction mixture was stirred at 25° C. for 50 minutes. The mixture was poured onto water (500 mL) and filtered. The filter cake was dissolved with tetrahydrofuran (600 mL) and extracted with brine (300 mL×2). The combined organic extracts were dried over anhydrous sodium sulfate, filtered, and concentrated. The residue was triturated with methyl tert-butyl ether (300 mL) to afford 7-chloro-2-methylsulfanyl-3H-pyrido[4,3-d]pyrimidin-4-one (14 g, 61.49 mmol, 77% yield) as a yellow solid. LC/MS (ESI) m/z: 228.0 [M+H]⁺. ¹H NMR (400 MHz, DMSO-d₆) δ: 13.38-12.63 (m, 1H), 8.95 (s, 1H), 7.51 (s, 1H), 2.58 (s, 3H).

Step 3: Preparation of 7-bromo-8-fluoro-1H-quinazoline-2,4-dione

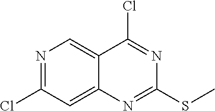

To a solution of 7-chloro-2-methylsulfanyl-3H-pyrido[4,3-d]pyrimidin-4-one (4 g, 17.57 mmol, 1 eq) in phosphoryl chloride (40 mL) at 0° C. was added diisopropylethylamine (4.54 g, 35.14 mmol, 6 mL, 2 eq), and the reaction mixture was stirred at 90° C. for 30 minutes. The mixture was concentrated to afford 4,7-dichloro-2-methylsulfanyl-pyrido[4,3-d]pyrimidine (4.3 g, crude) as a white solid.

Step 4: Preparation of tert-butyl 3-(7-chloro-2-methylsulfanyl-pyrido[4,3-d]pyrimidin-4-yl)-3,8-diazabicyclo[3.2.1]octane-8-carboxylate

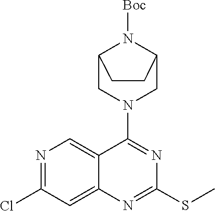 [SMILES: CSc1nc(N2CC3CCC(C2)N3C(=O)OC(C)(C)C)c2cnc(Cl)cc2n1]

To a solution of 4,7-dichloro-2-methylsulfanyl-pyrido[4,3-d]pyrimidine (4.3 g, 17.47 mmol, 1 eq) and tert-butyl 3,8-diazabicyclo[3.2.1]octane-8-carboxylate (3.71 g, 17.47 mmol, 1 eq) in dichloromethane (45 mL) at −60° C. was added diisopropylethylamine (11.29 g, 87.36 mmol, 15 mL, 5 eq), and the reaction mixture was stirred at −60° C. for 30 minutes. The mixture was diluted with water (100 mL) and extracted with dichloromethane (100 mL×2). The combined organic extracts were washed with brine (100 mL×2), dried over anhydrous sodium sulfate, filtered, and concentrated. The residue was purified by flash silica gel chromatography (gradient: 0˜16% ethyl acetate in petroleum ether) to afford tert-butyl 3-(7-chloro-2-methylsulfanyl-pyrido[4,3-d]pyrimidin-4-yl)-3,8-diazabicyclo[3.2.1]octane-8-carboxylate (2.5 g, 5.92 mmol, 34% yield) as a brown solid. ¹H NMR (400 MHz, DMSO-d₆) δ 9.08-9.02 (m, 1H), 7.59-7.46 (m, 1H), 4.48 (d, J=12.8 Hz, 2H), 4.23 (s, 2H), 3.62 (d, J=12.8 Hz, 2H), 2.52 (s, 3H), 1.78 (d, J=5.2 Hz, 2H), 1.60 (d, J=7.6 Hz, 2H), 1.46 (s, 9H).

Step 5: Preparation of tert-butyl 3-[7-[8-ethyl-3-(methoxymethoxy)-1-naphthyl]-2-methylsulfanyl-pyrido[4,3-d]pyrimidin-4-yl]-3,8-diazabicyclo[3.2.1]octane-8-carboxylate

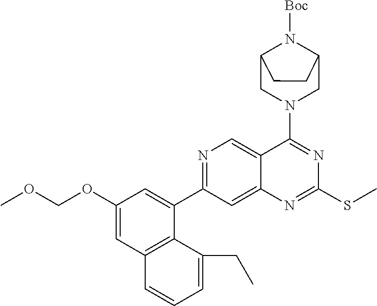

A mixture of tert-butyl 3-(7-chloro-2-methylsulfanyl-pyrido[4,3-d]pyrimidin-4-yl)-3,8-diazabicyclo[3.2.1]octane-8-carboxylate (2.5 g, 5.92 mmol, 1 eq), 2-[8-ethyl-3-(methoxymethoxy)-1-naphthyl]-4,4,5,5-tetramethyl-1,3,2-dioxaborolane (3.04 g, 8.89 mmol, mmol, 3 eq), and [2-(2-1.5 eq), potassium phosphate (3.77 g, 17.77 aminophenyl)phenyl]palladium (1+); bis(1-adamantyl)-butylphosphane; methanesulfonate (431.50 mg, 0.59 mmol, 0.1 eq) in dioxane (50 mL) and water (5 mL) was degassed, then stirred at 85° C. for 16 hours under nitrogen. The mixture was cooled to 25° C., diluted with ethyl acetate (400 mL), and washed with brine (80 mL×2). The combined organic extract was dried over anhydrous sodium sulfate, filtered, and concentrated. The residue was purified by silica gel column chromatography (Petroleum ether/Ethyl acetate=10/1 to 3:1) to afford tert-butyl 3-[7-[8-ethyl-3-(methoxymethoxy)-1-naphthyl]-2-methylsulfanyl-pyrido[4,3-d]pyrimidin-4-yl]-3,8-diazabicyclo[3.2.1]octane-8-carboxylate (3 g, 4.99 mmol, 84% yield) as a light yellow gum. LC/MS (ESI) m/z: 602.3 [M+H]⁺.

Step 6: tert-butyl 3-[7-[8-ethyl-3-(methoxymethoxy)-1-naphthyl]-2-methylsulfonyl-pyrido[4,3-d]pyrimidin-4-yl]-3,8-diazabicyclo[3.2.1]octane-8-carboxylate

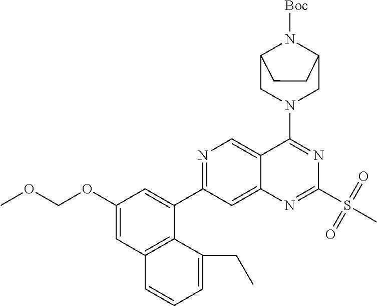

To a solution of tert-butyl 3-[7-[8-ethyl-3-(methoxymethoxy)-1-naphthyl]-2-methylsulfanyl-pyrido[4,3-d]pyrimidin-4-yl]-3,8-diazabicyclo[3.2.1]octane-8-carboxylate (1 g, 1.66 mmol, 1 eq) in DMF (3 mL) was added 4 Å MS (1 g), and the resulting mixture was stirred at 25° C. for 5 h. Oxone (3.06 g, 4.99 mmol, 3 eq) was then added, and the reaction mixture was stirred at 25° C. for 12 h. The reaction mixture was filtered, and the filtrate was washed with brine (50 mL), dried over anhydrous sodium sulfate, filtered, and concentrated. The residue was purified by flash silica gel chromatography (gradient: 0˜40% ethyl acetate in petroleum ether) to afford tert-butyl 3-[7-[8-ethyl-3-(methoxymethoxy)-1-naphthyl]-2-methylsulfonyl-pyrido[4,3-d]pyrimidin-4-yl]-3,8-diazabicyclo[3.2.1]octane-8-carboxylate (504 mg, 0.80 mmol, 48% yield) as a yellow solid. LC/MS (ESI) m/z: 634.3 [M+H]⁺.

Step 7: Preparation of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxy methoxy)-1-naphthyl]pyrido[4,3-d]pyrimidin-4-yl]-3,8-diazabicyclo[3.2.1]octane-8-carboxylate

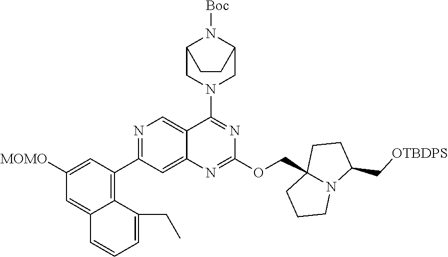

To a solution of tert-butyl 3-[7-[8-ethyl-3-(methoxymethoxy)-1-naphthyl]-2-methylsulfonyl-pyrido[4,3-d]pyrimidin-4-yl]-3,8-diazabicyclo[3.2.1]octane-8-carboxylate (504 mg, 0.80 mmol, 1 eq) and [(3S,8R)-3-[[tert-butyl(diphenyl)silyl]oxymethyl]-1,2,3,5,6,7-hexahydro pyrrolizin-8-yl]methanol (358 mg, 0.87 mmol, 1.1 eq) in tetrahydrofuran (5 mL) at 0° C. was added lithium tert-butoxide (2.2 M, 1.08 mL, 3 eq), and the reaction mixture was stirred at 25° C. for 12 h. The reaction mixture was diluted with water (50 mL) and extracted with ethyl acetate (15 mL×3). The combined organic extract was washed with brine (50 mL), dried over anhydrous sodium sulfate, filtered, and concentrated. The residue was purified by flash silica gel chromatography (gradient: 0˜40% ethyl acetate in petroleum ether) to afford tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]pyrido[4,3-d]pyrimidin-4-yl]-3,8-diazabicyclo[3.2.1]octane-8-carboxylate (475 mg, 0.49 mmol, 62% yield) as a yellow solid. LC/MS (ESI) m/z: 963.8 [M+H]⁺.

Step 8: Preparation of tert-butyl 3-[7-[8-ethyl-3-(methoxymethoxy)-1-naphthyl]-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate

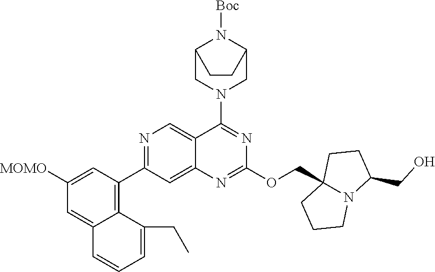

To a solution of tert-butyl 3-[2-[[(3S,8R)-3-[[tert-butyl(diphenyl)silyl]oxymethyl]-1,2,3,5,6,7-hexahydropyrrolizin-8-yl]methoxy]-7-[8-ethyl-3-(methoxymethoxy)-1-naphthyl]pyrido[4,3-d]pyrimidin-4-yl]-3,8-diazabicyclo[3.2.1]octane-8-carboxylate (475 mg, 0.49 mmol, 1 eq) in DMF (5 mL) was added cesium fluoride (1.50 g, 9.86 mmol, 0.4 mL, 20 eq), and the reaction mixture was stirred a 25° C. for 12 h. The reaction mixture was filtered, and the filtrate was diluted with water (100 mL) and extracted with ethyl acetate (20 mL×3). The combined organic extract was washed with brine (100 mL), dried over anhydrous sodium sulfate, filtered, and concentrated. The residue was purified by prep-TLC (dichloromethane/methanol=8/1) to afford tert-butyl 3-[7-[8-ethyl-3-(methoxy methoxy)-1-naphthyl]-2-[[(3S,8R)-3-(hydroxymethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate (322 mg, 0.44 mmol, 90% yield) as a yellow solid. LC/MS (ESI) m/z: 725.4 [M+H]⁺.

Step 9: Preparation of [(3S,8R)-8-[[4-(3,8-diazabicyclo[3.2.1]octan-3-yl)-7-(8-ethyl-3-hydroxy-1-naphthyl)pyrido[4,3-d]pyrimidin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

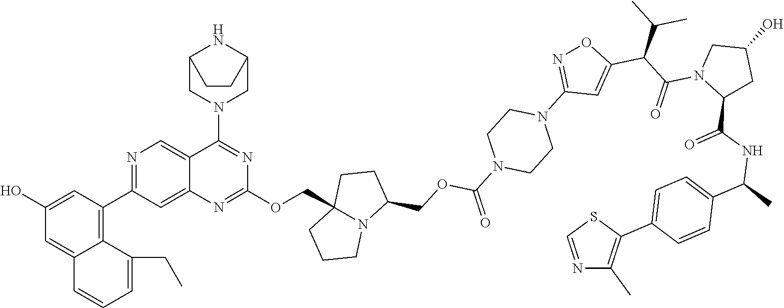 [SMILES: CCc1cccc2cc(O)cc(-c3cc4nc(OC[C@]56CCCN5[C@H](COC(=O)N5CCN(c7cc([C@H](C(=O)N8C[C@H](O)C[C@H]8C(=O)N[C@@H](C)c8ccc(-c9scnc9C)cc8)C(C)C)on7)CC5)CC6)nc(N5CC6CCC(C5)N6)c4cn3)c12]

The title compound was made in an analogous manner to [(3R,8S)-8-[[6-chloro-4-(3,8-diazabicyclo[3.2.1]octan-3-yl)-8-fluoro-7-(3-hydroxy-1-naphthyl) quinazolin-2-yl]oxymethyl]-1,2,3,5,6,7-hexahydropyrrolizin-3-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate starting from tert-butyl 3-[7-[8-ethyl-3-(methoxymethoxy)-1-naphthyl]-2-[[(3S,8R)-3-(hydroxylmethyl)-1,2,3,5,6,7-hexahydropyrrolizin-8-yl]methoxy]pyrido[4,3-d]pyrimidin-4-yl]-3,8-diazabicyclo[3.2.1]octane-8-carboxylate and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide. (formic acid salt, yellow solid). LC/MS (ESI) m/z: 1174.8 [M+H]⁺. ¹HNMR (400 MHz, DMSO-d₆) δ 9.94-9.69 (m, 1H), 9.19 (s, 1H), 8.98 (s, 1H), 8.40 (d, J=8.0 Hz, 1H), 8.21 (s, 1H), 7.63 (d, J=8.0 Hz, 1H), 7.46-7.40 (m, 3H), 7.39-7.31 (m, 3H), 7.22 (d, J=2.4 Hz, 1H), 7.11 (d, J=6.8 Hz, 1H), 6.94 (d, J=2.4 Hz, 1H), 6.17-6.04 (m, 1H), 4.96-4.85 (m, 1H), 4.44-4.26 (m, 4H), 4.09-3.84 (m, 4H), 3.75-3.67 (m, 1H), 3.63-3.54 (m, 6H), 3.47-3.45 (m, 3H), 3.16 (s, 5H), 3.00-2.89 (m, 3H), 2.45 (s, 3H), 2.26-2.14 (m, 3H), 2.07-1.90 (m, 4H), 1.84-1.75 (m, 4H), 1.69-1.57 (m, 6H), 1.44 (d, J=6.8 Hz, 1H), 1.37 (d, J=6.8 Hz, 3H), 1.27-1.20 (m, 1H), 0.98-0.92 (m, 3H), 0.83-0.76 (m, 6H).

Exemplary Synthesis of (2S,4R)-1-[(2R)-2-[3-[[(6R,8aR)-8a-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-2,3,5,6,7,8-hexahydro-1H-indolizin-6-yl]oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 318)

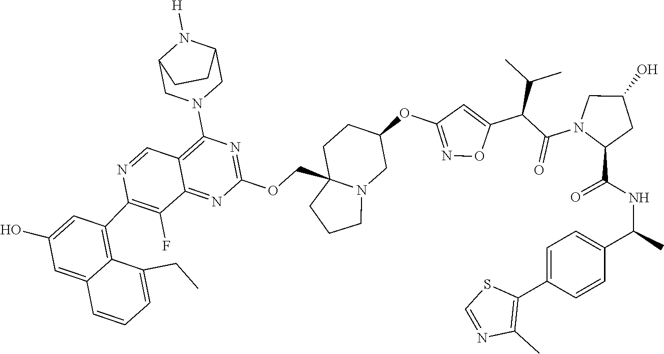 [SMILES: CCc1cccc2cc(O)cc(-c3ncc4c(N5CC6CCC(C5)N6)nc(OC[C@]56CCCN5C[C@H](Oc5cc([C@H](C(=O)N7C[C@H](O)C[C@H]7C(=O)N[C@@H](C)c7ccc(-c8scnc8C)cc7)C(C)C)on5)CC6)nc4c3F)c12]

The title compound was made in an analogous manner to (2S,4R)-1-[(2R)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from [(6R,8aR)-6-[5-(1-methoxycarbonyl-isobutyl)isoxazol-3-yl]oxy-2,3,5,6,7,8-hexahydro-1H-indolizin-8a-yl]methyl benzoate. (formic acid salt, white solid). LC/MS (ESI) m/z: 1079.9 [M+H]⁺. ¹H NMR (400 MHz, CD₃OD) δ 9.09 (s, 1H), 8.87 (s, 1H), 8.44 (s, 1H), 7.64 (d, J=7.6 Hz, 1H), 7.47-7.33 (m, 5H), 7.32-7.27 (m, 1H), 7.17 (d, J=6.8 Hz, 1H), 7.00 (dd, J=6.4, 2.4 Hz, 1H), 5.99-5.87 (m, 1H), 5.06-4.99 (m, 1H), 4.82-4.70 (m, 4H), 4.58-4.46 (m, 2H), 4.45-4.37 (m, 1H), 4.06 (d, J=10.0 Hz, 2H), 3.91 (d, J=13.2 Hz, 1H), 3.87-3.79 (m, 2H), 3.76-3.64 (m, 1H), 3.60 (d, J=10.8 Hz, 1H), 3.54-3.35 (m, 2H), 3.27-3.12 (m, 2H), 2.51-2.43 (m, 3H), 2.41-2.26 (m, 3H), 2.09-1.93 (m, 13H), 1.87-1.76 (m, 1H), 1.60-1.47 (m, 3H), 1.10-1.00 (m, 3H), 0.93-0.83 (m, 6H).

Exemplary Synthesis of (2S,4R)-1-[(2S)-2-[3-[[(6R,8aR)-8a-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-2,3,5,6,7,8-hexahydro-1H-indolizin-6-yl]oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 319)

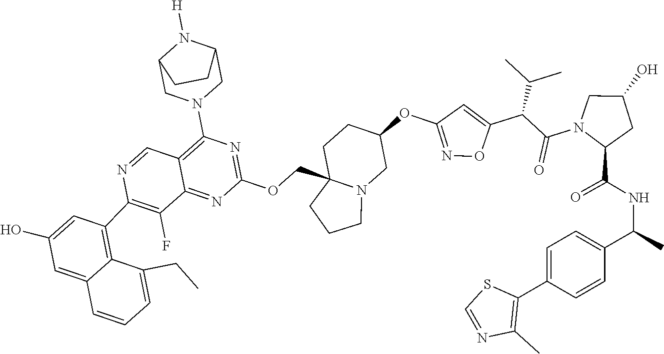 [SMILES: CCc1cccc2cc(O)cc(-c3ncc4c(N5CC6CCC(C5)N6)nc(OC[C@]56CCCN5C[C@H](Oc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was made in an analogous manner to (2S,4R)-1-[(2S)-2-[3-[[(3S,8R)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from [(6R,8aR)-6-[5-(1-methoxycarbonyl-isobutyl)isoxazol-3-yl]oxy-2,3,5,6,7,8-hexahydro-1H-indolizin-8a-yl]methyl benzoate. (formic acid salt, white solid). LC/MS (ESI) m/z: 1079.9 [M+H]⁺. ¹H NMR (400 MHz, CD₃OD) δ 9.07 (d, J=2.0 Hz, 1H), 8.90-8.80 (m, 1H), 8.47 (s, 1H), 7.62 (d, J=8.4 Hz, 1H), 7.48-7.27 (m, 6H), 7.15 (d, J=7.2 Hz, 1H), 7.02 (dd, J=2.4, 1.2 Hz, 1H), 6.04-5.94 (m, 1H), 5.01-4.95 (m, 1H), 4.81-4.67 (m, 4H), 4.56 (t, J=8.0 Hz, 1H), 4.52-4.45 (m, 1H), 4.42 (s, 1H), 4.01 (d, J=10.0 Hz, 2H), 3.91-3.74 (m, 3H), 3.74-3.61 (m, 2H), 3.38-3.32 (m, 1H), 3.26-3.08 (m, 3H), 2.50-2.42 (m, 3H), 2.39-2.19 (m, 4H), 2.14-1.81 (m, 12H), 1.79-1.69 (m, 1H), 1.60-1.42 (m, 3H), 1.08-0.93 (m, 3H), 0.93-0.81 (m, 6H).

Exemplary Synthesis of (2S,4R)-1-[(2R)-2-[3-[[1-[[(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 335)

Step 1: Preparation of tert-butyl 4-fluoro-4-[[5-(1-methoxy carbonyl-isobutyl)isoxazol-3-yl]oxymethyl]piperidine-1-carboxylate

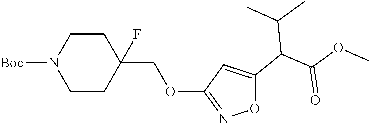 [SMILES: COC(=O)C(c1cc(OCC2(F)CCN(C(=O)OC(C)(C)C)CC2)no1)C(C)C]

To a mixture of methyl 2-(3-hydroxyisoxazol-5-yl)-isovalerate (500 mg, 2.51 mmol, 1.0 eq), tert-butyl 4-fluoro-4-(hydroxymethyl)piperidine-1-carboxylate (586 mg, 2.51 mmol, 1.0 eq), and PPh₃ (1.32 g, 5.02 mmol, 2.0 eq) in toluene (10 mL) at 0° C. was added DIAD (0.976 mL, 5.02 mmol, 2.0 eq), and the reaction mixture was stirred at 110° C. for 15 hours. The mixture was concentrated under reduced pressure, and the resulting residue was purified by flash chromatography on SiO₂ (gradient: 0-10% ethyl acetate in petroleum ether to afford tert-butyl 4-fluoro-4-[[5-(1-methoxycarbonyl-isobutyl)isoxazol-3-yl]oxymethyl]piperidine-1-carboxylate (928 mg, 1.93 mmol, 77% yield) as a yellow oil. LC/MS (ESI) m/z: 415.1 [M+H]⁺. ¹H NMR (400 MHz, CDCl₃) δ 5.95 (s, 1H), 4.25 (d, J=19.2 Hz, 2H), 4.09-3.87 (m, 2H), 3.74 (s, 3H), 3.50 (d, J=8.8 Hz, 1H), 3.21-3.06 (m, 2H), 2.36 (quint, J=8.8, 6.8 Hz, 1H), 2.01-1.90 (m, 2H), 1.78-1.61 (m, 2H), 1.47 (s, 9H), 1.01 (d, J=6.8 Hz, 3H), 0.93 (d, J=6.8 Hz, 3H).

Step 2: Preparation of methyl 2-[3-[(4-fluoro-4-piperidyl) meth oxy]isoxazol-5-yl]-isovalerate

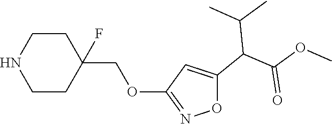 [SMILES: COC(=O)C(c1cc(OCC2(F)CCNCC2)no1)C(C)C]

To tert-butyl 4-fluoro-4-[[5-(1-methoxycarbonyl-isobutyl)isoxazol-3-yl]oxymethyl]piperidine-1-carboxylate (828 mg, 86% purity, 1.72 mmol) in CH₂Cl₂ (10 mL) was added 4 M HCl/dioxane (8.0 mL), and the reaction was stirred at 20° C. for 30 min. The mixture was concentrated under reduced pressure to remove CH₂Cl₂, and saturated aqueous NaHCO₃ was then added to adjust the pH (pH ˜8). The resulting mixture was extracted with 10/1 CH₂Cl₂/MeOH (3×30 mL), and the combined extracts were dried over anhydrous Na₂SO₄, filtered, and concentrated under reduced pressure to afford methyl 2-[3-[(4-fluoro-4-piperidyl)methoxy]isoxazol-5-yl]-isovalerate (658 mg, 1.61 mmol, 94% yield) as a colorless oil. LC/MS (ESI) m/z: 315.1 [M+H]⁺.

Step 3: Preparation of methyl 2-[3-[[1-[[(3S,8S)-8-[[tert-butyl(diphenyl)silyl]oxymethyl]-1,2,3,5,6,7-hexahydropyrrolizin-3-yl]methyl]-4-fluoro-4-piperidyl]methoxy]isoxazol-5-yl]-isovalerate

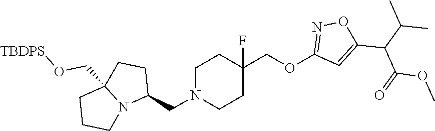

To a solution of (3S,8S)-8-[[tert-butyl(diphenyl)silyl]oxymethyl]-1,2,3,5,6,7-hexahydropyrrolizine-3-carbaldehyde (410 mg, 1.01 mmol, 1.12 eq) and methyl 2-[3-[(4-fluoro-4-piperidyl)methoxy]isoxazol-5-yl]-isovalerate (370 mg, 77% purity, 0.905 mmol, 1.0 eq) in CH₂Cl₂ (10 mL) was added NaBH(OAc)₃ (469 mg, 2.21 mmol, 2.44 eq), and the reaction mixture was stirred at 20° C. for 15 hours. Saturated aqueous NaHCO₃ solution (10 mL) was then added followed by with water (10 mL), and the resulting mixture was extracted with CH₂Cl₂ (3×20 mL). The combined organic extracts were dried over anhydrous Na₂SO₄, filtered, and concentrated under reduced pressure. The residue was purified by flash chromatography on SiO₂ (gradient: 0-10% CH₃OH in CH₂Cl₂) to afford methyl 2-[3-[[1-[[(3S,8S)-8-[[tert-butyl(diphenyl)silyl]oxymethyl]-1,2,3,5,6,7-hexahydropyrrolizin-3-yl]methyl]-4-fluoro-4-piperidyl]methoxy]isoxazol-5-yl]-isovalerate (703 mg, 0.706 mmol, 78% yield) as a yellow oil. LC/MS (ESI) m/z: 706.3 [M+H]⁺.

Step 4: Preparation of methyl 2-[3-[[4-fluoro-1-[[(3S,8S)-8-(hydroxymethyl)-1,2,3,5,6,7-hexahydropyrrolizin-3-yl]methyl]-4-piperidyl]methoxy]isoxazol-5-yl]-isovalerate

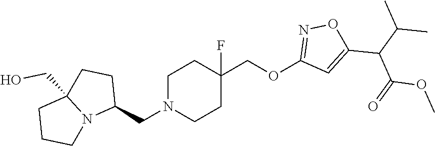 [SMILES: COC(=O)C(c1cc(OCC2(F)CCN(C[C@@H]3CC[C@]4(CO)CCCN34)CC2)no1)C(C)C]

To a solution of methyl 2-[3-[[1-[[(3S,8S)-8-[[tert-butyl(diphenyl)silyl]oxymethyl]-1,2,3,5,6,7-hexahydropyrrolizin-3-yl]methyl]-4-fluoro-4-piperidyl]methoxy]isoxazol-5-yl]-isovalerate (703 mg, 70.9% purity, 0.706 mmol, 1.0 eq) in CH₂Cl₂ (15 mL) was added trimethylamine trihydrofluoride (1.15 mL, 7.06 mmol, 10 eq), and the reaction mixture was stirred at 20° C. for 1 hour. Saturated aqueous NaHCO₃ solution was added until pH=8, and the resulting mixture was extracted with 10/2 CH₂Cl/CH₃OH (3×25 mL). The combined organic extracts were dried over anhydrous Na₂SO₄, filtered, and concentrated under reduced pressure. The crude product was purified by preparative HPLC {Column: Phenomenex C18 75*30 mm*3 um; Mobile phase: [0-40% CH₃CN in water (formic acid)]}. Pure fraction was lyophilized to afford methyl 2-[3-[[4-fluoro-1-[[(3S,8S)-8-(hydroxymethyl)-1,2,3,5,6,7-hexahydropyrrolizin-3-yl]methyl]-4-piperidyl]methoxy]isoxazol-5-yl]-isovalerate (278 mg, 0.476 mmol, 67% yield, formic acid salt) as a colorless oil. LC/MS (ESI) m/z: 468.3 [M+H]⁺.

Step 5: Preparation of (2S,4R)-1-[(2R)-2-[3-[[1-[[(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

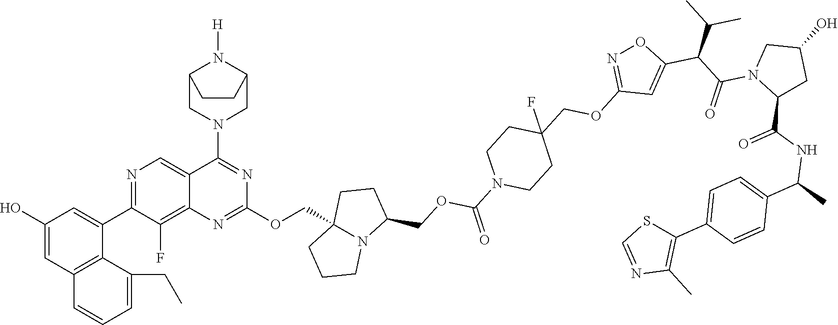 [SMILES: CCc1cccc2cc(O)cc(-c3ncc4c(N5CC6CCC(C5)N6)nc(OC[C@@]56CCCN5[C@H](COC(=O)N5CCC(F)(COc7cc([C@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was made in an analogous manner to (2S,4R)-1-[(2R)-2-[3-[7-[[(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from methyl 2-[3-[[4-fluoro-1-[[(3S,8S)-8-(hydroxymethyl)-1,2,3,5,6,7-hexahydropyrrolizin-3-yl]methyl]-4-piperidyl]methoxy]isoxazol-5-yl]-isovalerate. (formic acid salt, white solid). LC/MS (ESI) m/z: 1195.0 [M+H]⁺. ¹H NMR (400 MHz, DMSO-d₆) δ 9.09 (s, 1H), 9.01-8.82 (m, 1H), 8.43 (d, J=7.6 Hz, 1H), 7.66 (d, J=8.0 Hz, 1H), 7.49-7.24 (m, 6H), 7.11 (d, J=7.2 Hz, 1H), 6.97 (s, 1H), 6.19-5.95 (m, 1H), 4.90 (quin, J=6.8 Hz, 1H), 4.54-4.10 (m, 10H), 3.48-3.34 (m, 10H), 2.85-2.57 (m, 5H), 2.45 (s, 3H), 2.28-2.00 (m, 7H), 1.90-1.52 (m, 16H), 1.47-1.33 (m, 3H), 0.95 (d, J=6.0 Hz, 3H), 0.86-0.74 (m, 6H).

Exemplary Synthesis of (2S,4R)-1-[(2S)-2-[3-[[1-[[(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 336)

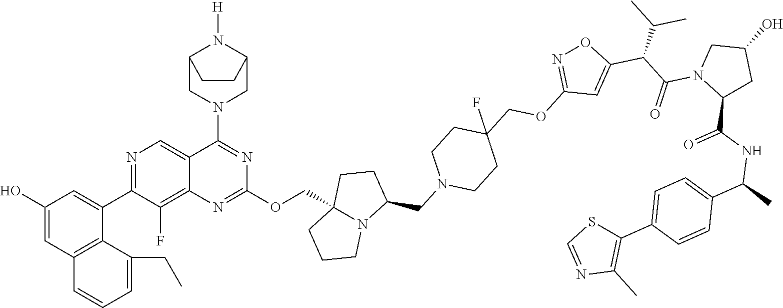 [SMILES: CCc1cccc2cc(O)cc(-c3ncc4c(N5CC6CCC(C5)N6)nc(OC[C@@]56CCCN5[C@H](CN5CCC(F)(COc7cc([C@@H](C(=O)N8C[C@H](O)C[C@H]8C(=O)N[C@@H](C)c8ccc(-c9scnc9C)cc8)C(C)C)on7)CC5)CC6)nc4c3F)c12]

The title compound was made in an analogous manner to (2S,4R)-1-[(2R)-2-[3-[7-[[(3S,8S)-8-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1195.0 [M+H]⁺. ¹H NMR (400 MHz, DMSO-d₆) δ 9.09 (d, J=1.6 Hz, 1H), 9.01-8.93 (m, 1H), 8.34 (d, J=7.6 Hz, 1H), 7.65 (d, J=8.4 Hz, 1H), 7.54-7.20 (m, 6H), 7.11 (d, J=7.2 Hz, 1H), 6.96 (d, J=2.4 Hz, 1H), 6.22-6.10 (m, 1H), 5.04-4.81 (m, 1H), 4.61-4.05 (m, 9H), 3.81-3.66 (m, 5H), 3.33 (d, J=9.6 Hz, 6H), 2.88-2.55 (m, 5H), 2.46-2.42 (m, 3H), 2.31-2.03 (m, 7H), 1.92-1.52 (m, 16H), 1.47-1.30 (m, 3H), 1.00-0.71 (m, 9H).

Exemplary Synthesis of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 320)

Step 1: Preparation of tert-butyl 4-[[1-[5-(1-methoxycarbonyl-2-methyl-propyl)isoxazol-3-yl]-4-piperidyl]methyl]piperidine-1-carboxylate

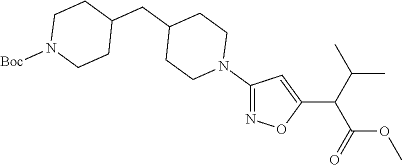 [SMILES: COC(=O)C(c1cc(N2CCC(CC3CCN(C(=O)OC(C)(C)C)CC3)CC2)no1)C(C)C]

To a solution of tert-butyl 4-(4-piperidylmethyl)piperidine-1-carboxylate (7.34 g, 25.97 mmol, 1 eq) in N,N-dimethylacetamide (80 mL) were added triethylamine (7.88 g, 77.92 mmol, 10.8 mL, 3 eq) and methyl 3-methyl-2-[3-(1,1,2,2,3,3,4,4,4-nonafluorobutylsulfonyloxy)isoxazol-5-yl]butanoate (15 g, 31.17 mmol, 1.2 eq), and the reaction mixture was stirred at 130° C. for 12 h. The mixture was diluted with water (300 mL) and extracted with ethyl acetate (100 mL×2). The organic extract was washed with brine (100 mL×3), dried over sodium sulfate, filtered, and concentrated. The residue was purified by silica gel chromatography (petroleum ether/ethyl acetate=10/1 to 4:1) to afford tert-butyl 4-[[1-[5-(1-methoxycarbonyl-2-methyl-propyl)isoxazol-3-yl]-4-piperidyl]methyl]piperidine-1-carboxylate (2.7 g, 5.82 mmol, 22% yield) as a yellow solid. LC/MS (ESI) m/z: 464.3 [M+H]⁺. ¹H NMR (400 MHz, DMSO-d₆) δ 6.21 (s, 1H), 3.97-3.82 (m, 2H), 3.68-3.50 (m, 6H), 2.78-2.58 (m, 4H), 2.35-2.16 (m, 1H), 1.70-1.56 (m, 4H), 1.55-1.43 (m, 2H), 1.38 (s, 9H), 1.15-1.03 (m, 4H), 0.95-0.79 (m, 8H).

Step 2: Preparation of 2-[3-[4-[(1-tert-butoxycarbonyl-4-piperidyl)methyl]-1-piperidyl]isoxazol-5-yl]-3-methyl-butanoic acid

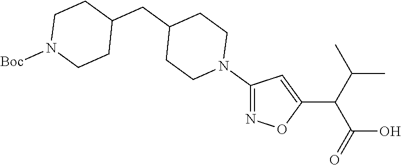 [SMILES: CC(C)C(C(=O)O)c1cc(N2CCC(CC3CCN(C(=O)OC(C)(C)C)CC3)CC2)no1]

To a solution of tert-butyl 4-[[1-[5-(1-methoxycarbonyl-2-methyl-propyl)isoxazol-3-yl]-4-piperidyl]methyl]piperidine-1-carboxylate (2.7 g, 5.82 mmol, 1 eq) in tetrahydrofuran (20 mL), water (20 mL), and methanol (20 mL) was added lithium hydroxide monohydrate (2.93 g, 69.89 mmol, 12 eq), and the reaction mixture was stirred at 25° C. for 1 h. The reaction mixture was adjust to pH=5 with 1 M hydrochloric acid and then extracted with ethyl acetate (100 mL×2). The combined organic extract was washed with brine (100 mL×2), dried over anhydrous sodium sulfate, filtered, and concentrated to afford 2-[3-[4-[(1-tert-butoxycarbonyl-4-piperidyl)methyl]-1-piperidyl]isoxazol-5-yl]-3-methyl-butanoic acid (2 g, crude) as a yellow solid. LC/MS (ESI) m/z: 450.4 [M+H]⁺. ¹H NMR (400 MHz, DMSO-d₆) δ: 6.16 (s, 1H), 3.97-3.84 (m, 2H), 3.67-3.57 (m, 2H), 3.42-3.37 (m, 1H), 2.80-2.58 (m, 4H), 2.30-2.16 (m, 1H), 1.68-1.44 (m, 6H), 1.38 (s, 9H), 1.16-1.04 (m, 4H), 1.00-0.79 (m, 8H).

Step 3: Preparation of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

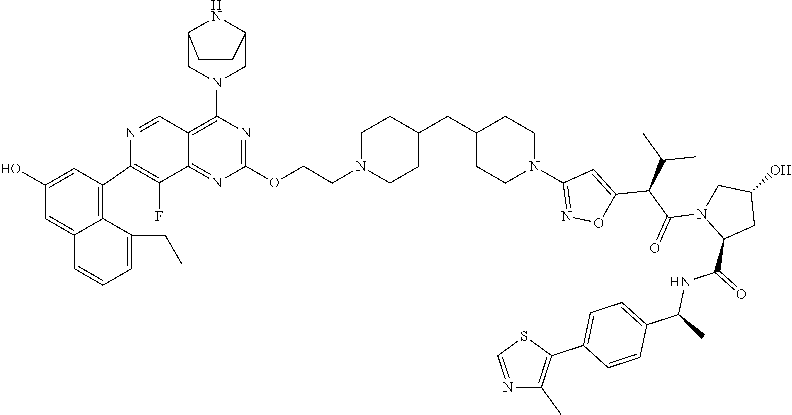 [SMILES: CCc1cccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from 2-[3-[4-[(1-tert-butoxycarbonyl-4-piperidyl)methyl]-1-piperidyl]isoxazol-5-yl]-3-methyl-butanoic acid (2 g, 4.45 mmol, 1 eq) and (2S,4R)-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1134.8 [M+H]⁺. ¹H NMR (400 MHz, DMSO-d₆) δ 9.13 (s, 1H), 8.98 (s, 1H), 8.84-8.35 (m, 1H), 8.21-8.11 (m, 1H), 7.72-7.63 (m, 1H), 7.49-7.25 (m, 6H), 7.17-7.10 (m, 1H), 7.00-6.92 (m, 1H), 6.15-5.92 (m, 1H), 5.22-5.00 (m, 1H), 4.98-4.84 (m, 1H), 4.63-4.45 (m, 4H), 4.40-4.21 (m, 2H), 4.00-3.92 (m, 2H), 3.86-3.66 (m, 4H), 3.62-3.51 (m, 4H), 3.08-2.99 (m, 2H), 2.89-2.80 (m, 2H), 2.76-2.64 (m, 3H), 2.45 (s, 3H), 2.28-2.14 (m, 5H), 2.07-1.96 (m, 1H), 1.92-1.76 (m, 5H), 1.69-1.59 (m, 4H), 1.53-1.34 (m, 5H), 1.17-1.03 (m, 6H), 1.01-0.91 (m, 3H), 0.85-0.74 (m, 6H).

Exemplary Synthesis of (2S,4R)-1-[(2S)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 321)

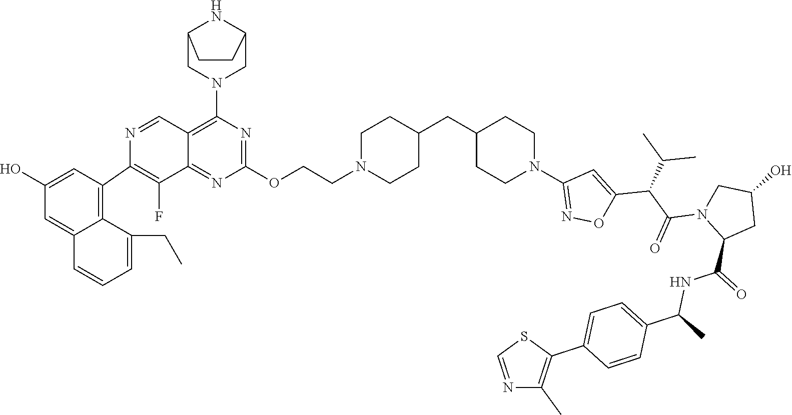 [SMILES: CCc1cccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1134.8 [M+H]⁺. ¹H NMR (400 MHz, DMSO-d₆) δ 9.12 (s, 1H), 9.03-8.97 (m, 1H), 8.95-8.10 (m, 1H), 7.73-7.61 (m, 1H), 7.50-7.27 (m, 6H), 7.18-7.08 (m, 1H), 7.02-6.91 (m, 1H), 6.12 (s, 1H), 5.18-4.81 (m, 2H), 4.60-4.47 (m, 4H), 4.44-4.35 (m, 1H), 4.32-4.22 (m, 1H), 3.98-3.86 (m, 2H), 3.78-3.54 (m, 8H), 3.06-2.96 (m, 2H), 2.86-2.61 (m, 5H), 2.44 (s, 3H), 2.30-2.04 (m, 6H), 1.90-1.74 (m, 5H), 1.69-1.56 (m, 4H), 1.48-1.31 (m, 5H), 1.14-0.94 (m, 8H), 0.89-0.71 (m, 7H).

Exemplary Synthesis of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 276)

Step 1: Preparation of tert-butyl 4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate

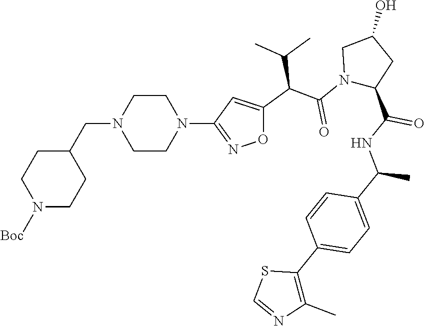 [SMILES: Cc1ncsc1-c1ccc([C@H](C)NC(=O)[C@@H]2C[C@@H](O)CN2C(=O)[C@@H](c2cc(N3CCN(CC4CCN(C(=O)OC(C)(C)C)CC4)CC3)no2)C(C)C)cc1]

To a solution of (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide (400 mg, 705.82 umol, 1.0 eq) and tert-butyl 4-formylpiperidine-1-carboxylate (301 mg, 1.41 mmol, 2.0 eq) in CH₂Cl₂ (10 mL) were added HOAc (212 mg, 3.53 mmol, 5.0 eq) and 2-picoline borane complex (377 mg, 3.53 mmol, 5.0 eq), and the reaction mixture was stirred at 25° C. for 2 hours. The mixture was concentrated, and the resulting residue was purified by flash chromatography on SiO₂ (gradient: 0˜7% MeOH in CH₂Cl₂) to afford tert-butyl 4-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (550 mg, 719.91 umol, 92% yield) as a white solid. LC/MS (ESI) m/z: 764.3 [M+H]⁺.

Step 2: Preparation of (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(4-methylthiazol-5-yl)phenyl]ethyl]pyrrolidine-2-carboxamide

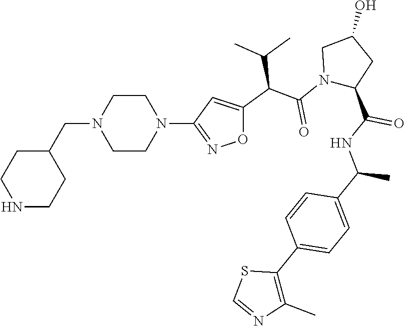 [SMILES: Cc1ncsc1-c1ccc([C@H](C)NC(=O)[C@@H]2C[C@@H](O)CN2C(=O)[C@@H](c2cc(N3CCN(CC4CCNCC4)CC3)no2)C(C)C)cc1]

To a solution of tert-butyl 4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (550 mg, 719.91 umol, 1.0 eq) in CH₂Cl₂ (8 mL) was added HCl/dioxane (4 M, 5 mL), and the reaction mixture was stirred at 25° C. for 1 hour. Petroleum ether (50 mL) was added, and the organic layer was discarded. The remaining material was diluted with THF (30 mL), treated with triethylamine, then concentrated under reduced pressure to afford (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(4-methylthiazol-5-yl)phenyl]ethyl]pyrrolidine-2-carboxamide (400 mg, crude) as a white solid. LC/MS (ESI) m/z: 664.3. [M+H]⁺.

Step 3: Preparation of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

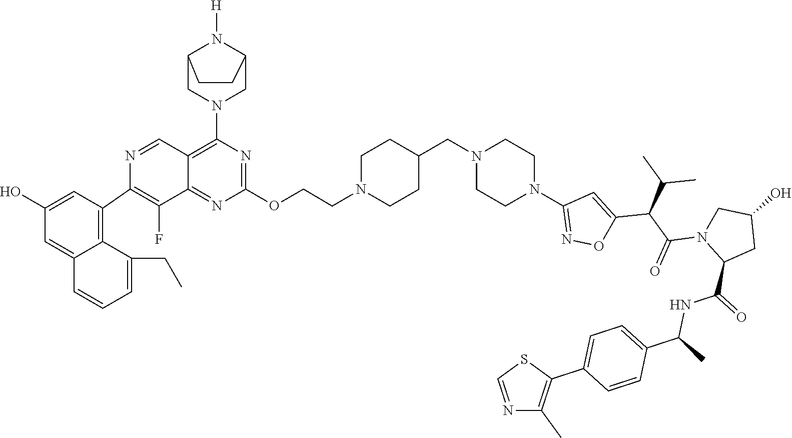 [SMILES: CCc1cccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1135.9 [M+H]⁺. ¹H NMR (400 MHz, CD₃OD) δ 9.12 (s, 1H), 8.87 (s, 1H), 7.64 (d, J=8.0 Hz, 1H), 7.45-7.35 (m, 5H), 7.30 (d, J=2.8 Hz, 1H), 7.17 (d, J=7.6 Hz, 1H), 7.01 (d, J=2.8 Hz, 1H), 6.11 (s, 1H), 5.03 (q, J=6.8 Hz, 1H), 4.79-4.74 (m, 3H), 4.51 (t, J=8.0 Hz, 1H), 4.44 (s, 1H), 4.12 (d, J=8.2 Hz, 1H), 3.97-3.83 (m, 3H), 3.67-3.63 (m, 3H), 3.60-3.47 (m, 3H), 3.25 (t, J=2.0 Hz, 4H), 2.99 (t, J=12.4 Hz, 2H), 2.55 (t, J=4.8 Hz, 4H), 2.48-2.47 (m, 3H), 2.40-1.89 (m, 15H), 1.59-1.44 (m, 5H), 1.05 (d, J=4.4 Hz, 3H), 0.92-0.87 (m, 6H).

Exemplary Synthesis of (2S,4R)-1-((R)-2-(3-(4-((1-(2-((4-((1R,5S)-3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl) piperidin-4-yl)methyl) piperidin-1-yl) isoxazol-5-yl)-3-methylbutanoyl)-4-hydroxy-N—((S)-1-(4-(4-methylthiazol-5-yl)phenyl)ethyl)pyrrolidine-2-carboxamide (Compound 307)

Step 1: Preparation of tert-butyl(1R,5S)-3-(8-fluoro-7-(7-fluoro-3-(methoxymethoxy)-8-((triisopropylsilyl) ethynyl)naphthalen-1-yl)-2-(2-(4-((1-(5-((R)-1-((2S,4R)-4-hydroxy-2-(((S)-1-(4-(4-methylthiazol-5-yl)phenyl)ethyl)carbamoyl) pyrrole din-1-yl)-3-methyl-1-oxobutan-2-yl)isoxazol-3-yl) piperidin-4-yl)methyl) piperidin-1-yl) ethoxy)pyrido[4,3-d]pyrimidin-4-yl)-3,8-diazabicyclo[3.2.1]octane-8-carboxylate

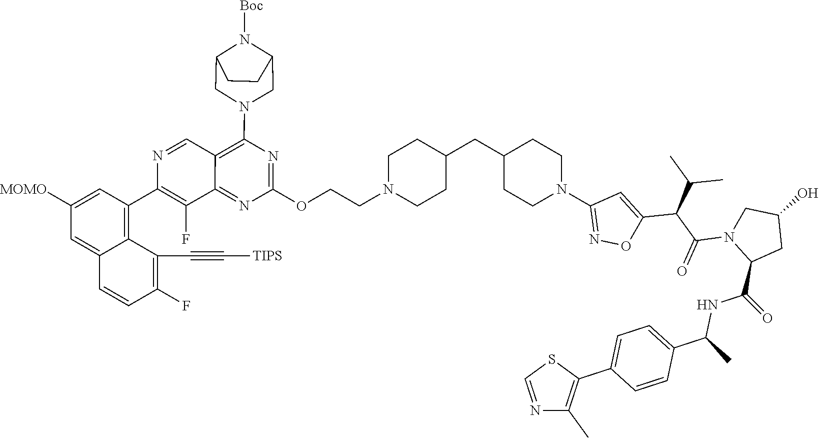

To a solution of (2S,4R)-4-hydroxy-1-[(2R)-3-methyl-2-[3-[4-(4-piperidylmethyl)-1-piperidyl]isoxazol-5-yl]butanoyl]-N-[(1S)-1-[4-(4-methylthiazol-5-yl)phenyl]ethyl]pyrrolidine-2-carboxamide (1.7 g, 2.19 mmol, 1 eq, trifluoroacetate) in dichloromethane (20 mL) and isopropanol (20 mL) was added N,N-diisopropylethylamine (1.41 g, 10.94 mmol, 1.9 mL, 5 eq), and the resulting mixture was stirred at 25° C. for 10 min. tert-Butyl (1R,5S)-3-(8-fluoro-7-(7-fluoro-3-(methoxymethoxy)-8-((triisopropylsilyl) ethynyl)naphthalen-1-yl)-2-(2-oxoethoxy)pyrido[4,3-d]pyrimidin-4-yl)-3,8-diazabicyclo[3.2.1]octane-8-carboxylate (2.11 g, 2.63 mmol, 1.2 eq) was then added, and the resulting mixture was stirred for 20 minutes. Sodium triacetoxyborohydride (1.39 g, 6.56 mmol, 3 eq) was then added, and the reaction mixture was stirred at 25° C. for 1 h. The mixture was diluted with water (30 mL) and extracted with ethyl acetate (30 mL×2). The combined organic extracts were washed with brine (30 mL×2), dried over anhydrous sodium sulfate, filtered, and concentrated. The resulting residue was purified by flash silica gel chromatography (gradient: 0˜100% ethyl acetate in petroleum ether to 0˜13% CH₃OH in CH₂Cl₂) to afford tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (3.7 g, crude) as a yellow solid. LC/MS (ESI) m/z: 1449.6 [M+H]⁺.

Step 2: Preparation of tert-butyl(1R,5S)-3-(7-(8-ethynyl-7-fluoro-3-(methoxymethoxy)naphthalen-1-yl)-8-fluoro-2-(2-(4-((1-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperidin-4-yl)methyl) piperidin-1-yl) ethoxy)pyrido[4,3-d]pyrimidin-4-yl)-3,8-diazabicyclo[3.2.1]octane-8-carboxylate

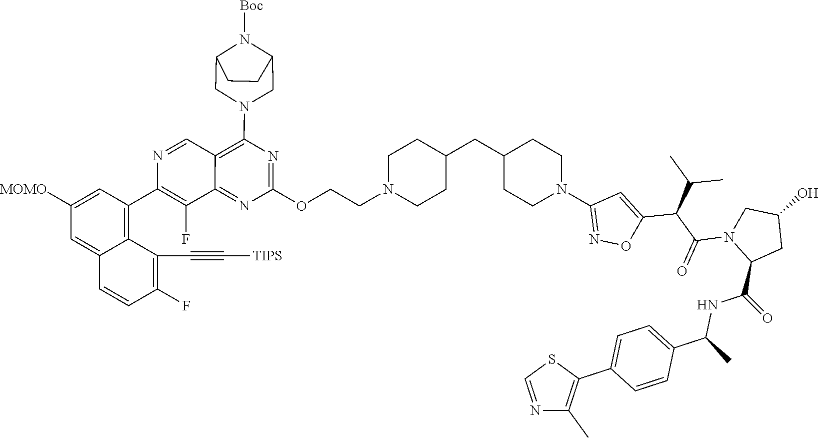

To a solution of tert-butyl(1R,5S)-3-(8-fluoro-7-(7-fluoro-3-(methoxymethoxy)-8-((triisopropylsilyl) ethynyl)naphthalen-1-yl)-2-(2-(4-((1-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperidin-4-yl)methyl) piperidin-1-yl) ethoxy)pyrido[4,3-d]pyrimidin-4-yl)-3,8-diazabicyclo[3.2.1]octane-8-carboxylate (3.7 g, 2.55 mmol, 1 eq) in DMF (40 mL) was added cesium fluoride (5.82 g, 38.31 mmol, 1.4 mL, 15 eq), and the reaction mixture was stirred at 25° C. for 12 h. The mixture was diluted with water (100 mL) and extracted with ethyl acetate (100 mL×2). The combined organic extracts were washed with brine (100 mL×2), dried over anhydrous sodium sulfate, filtered, and concentrated. The resulting residue was purified by prep-HPLC (column: UniSil 10-120 C18 70×250 mm; mobile phase: [46-76% CH₃CN in water (formic acid)]) to afford tert-butyl(1R,5S)-3-(7-(8-ethynyl-7-fluoro-3-(methoxymethoxy)naphthalen-1-yl)-8-fluoro-2-(2-(4-((1-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperidin-4-yl)methyl) piperidin-1-yl) ethoxy)pyrido[4,3-d]pyrimidin-4-yl)-3,8-diazabicyclo[3.2.1]octane-8-carboxylate (1.9 g, 1.47 mmol, 58% yield) as a yellow solid. LC/MS (ESI) m/z: 1293.6 [M+H]⁺.

Step 3: Preparation of (2S,4R)-1-((R)-2-(3-(4-((1-(2-((4-((1R,5S)-3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl) piperidin-4-yl)methyl) piperidin-1-yl) isoxazol-5-yl)-3-methylbutanoyl)-4-hydroxy-N—((S)-1-(4-(4-methylthiazol-5-yl)phenyl)ethyl)pyrrolidine-2-carboxamide

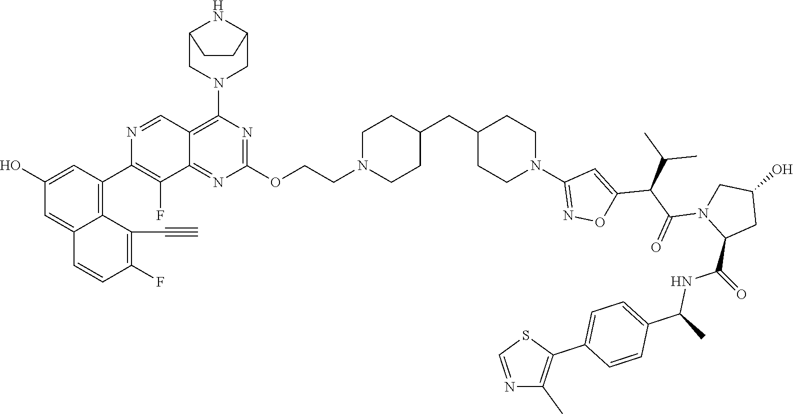 [SMILES: C#Cc1c(F)ccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To tert-butyl(1R,5S)-3-(7-(8-ethynyl-7-fluoro-3-(methoxymethoxy)naphthalen-1-yl)-8-fluoro-2-(2-(4-((1-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl) piperidin-4-yl)methyl) piperidin-1-yl) ethoxy)pyrido[4,3-d]pyrimidin-4-yl)-3,8-diazabicyclo[3.2.1]octane-8-carboxylate (1.9 g, 1.47 mmol, 1 eq) in dichloromethane (20 mL) was added hydrochloric acid/dioxane (4 M, 15 mL, 40.82 eq), and the reaction mixture was stirred at 25° C. for 15 minutes. The mixture was washed with petroleum ether (100 mL×2), and the remaining lower layers solid was taken up in tetrahydrofuran (50 mL). Triethylamine was then added to pH=7.00, and the suspension was filtered. The filtrate was concentrated, and the resulting residue was purified by prep-HPLC (column: Phenomenex luna C18 (250*70 mm, 10 um); mobile phase: [10-40% CH₃CN in water (formic acid)]) followed by prep-HPLC (column: Waters Xbridge 150*25 mm*5 um; mobile phase: [35-65% CH₃CN in water (NH₄OH)]). A third purification by prep-HPLC (column: Phenomenex luna C18 150*25 mm*10 um; mobile phase: [10-40% CH₃CN in water (formic acid)]) afforded (2S,4R)-1-((R)-2-(3-(4-((1-(2-((4-((1R,5S)-3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl) piperidin-4-yl)methyl) piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (385.6 mg, 0.36 mmol, 22% yield, formic acid salt) as a white solid. LC/MS (ESI) m/z: 1149.9 [M+H]⁺. ¹H NMR (400 MHz, CD₃OD) δ 9.07 (s, 1H), 8.93-8.83 (m, 1H), 8.50 (s, 1H), 7.87 (dd, J=6.0, 9.2 Hz, 1H), 7.47-7.40 (m, 4H), 7.39-7.35 (m, 2H), 7.34-7.30 (m, 1H), 7.21 (d, J=2.4 Hz, 1H), 6.10-6.00 (m, 1H), 5.10-4.98 (m, 1H), 4.82-4.73 (m, 3H), 4.68 (t, J=14.0 Hz, 2H), 4.51 (t, J=8.4 Hz, 1H), 4.47-4.38 (m, 1H), 3.92-3.85 (m, 2H), 3.84-3.73 (m, 3H), 3.69-3.58 (m, 4H), 3.58-3.42 (m, 3H), 3.41-3.33 (m, 3H), 2.88-2.73 (m, 4H), 2.50-2.45 (m, 3H), 2.41-2.29 (m, 1H), 2.24-2.13 (m, 1H), 2.05-1.84 (m, 7H), 1.77-1.62 (m, 3H), 1.61-1.49 (m, 4H), 1.48-1.34 (m, 2H), 1.28-1.15 (m, 4H), 1.05 (d, J=6.4 Hz, 3H), 0.94-0.84 (m, 3H).

Exemplary Synthesis of (2S,4R)-1-[(2S)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 308)

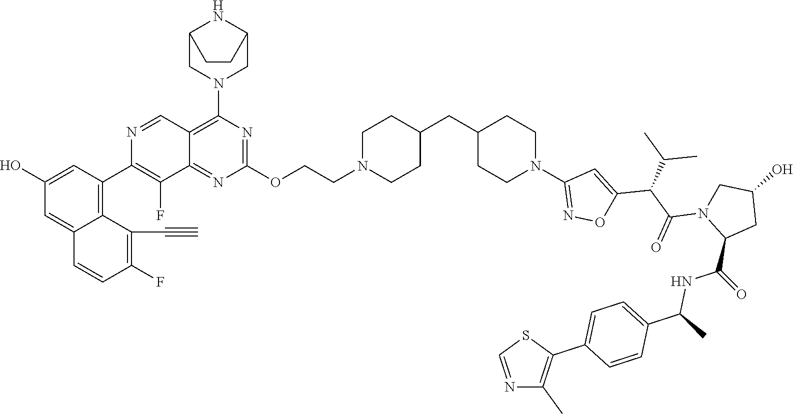 [SMILES: C#Cc1c(F)ccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-((R)-2-(3-(4-((1-(2-((4-((1R,5S)-3,8-diazabicyclo[3.2.1]octan-3-yl)-7-(8-ethynyl-7-fluoro-3-hydroxynaphthalen-1-pyrimidin-2-yl)oxy)ethyl) piperidin-4-yl)methyl) piperidin-1-yl)-8-fluoropyrido[4,3-d]yl)isoxazol-5-yl)-3-methylbutanoyl)-4-hydroxy-N—((S)-1-(4-(4-methylthiazol-5-yl)phenyl)ethyl)pyrrolidine-2-carboxamide starting from (2S,4R)-4-hydroxy-1-[(2S)-3-methyl-2-[3-[4-(4-piperidylmethyl)-1-piperidyl]isoxazol-5-yl]butanoyl]-N-[(1S)-1-[4-(4-methylthiazol-5-yl)phenyl]ethyl]pyrrolidine-2-carboxamide and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate. (yellow solid). LC/MS (ESI) m/z: 1148.2 [M+H]⁺. ¹H NMR (400 MHz, DMSO-d₆) δ 10.23-10.09 (m, 1H), 9.07-8.95 (m, 2H), 8.94-8.14 (m, 1H), 8.03-7.92 (m, 1H), 7.51-7.30 (m, 6H), 7.20-7.14 (m, 1H), 6.12 (s, 1H), 5.13-4.82 (m, 2H), 4.52-4.38 (m, 4H), 4.36-4.20 (m, 2H), 3.93 (s, 1H), 3.72-3.49 (m, 9H), 2.97-2.89 (m, 2H), 2.70-2.66 (m, 4H), 2.44 (s, 3H), 2.29-2.15 (m, 2H), 2.04-1.94 (m, 3H), 1.85-1.75 (m, 1H), 1.70-1.64 (m, 4H), 1.63-1.54 (m, 4H), 1.48-1.30 (m, 5H), 1.14-1.01 (m, 6H), 0.97-0.92 (m, 2H), 0.87-0.71 (m, 4H).

Exemplary Synthesis of (2S,4R)-1-[(2R)-2-[3-[[1-[2-[[4-(3,8-diazabicyclo[3.2.1]octan-3-yl)-7-(5-ethyl-4-isoquinolyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 304)

Step 1: Preparation of (5-chloro-4-isoquinolyl)-trimethyl-stannane

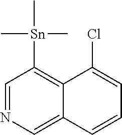

To a solution of 4-bromo-5-chloro-isoquinoline (900 mg, 3.71 mmol, 1 eq) in toluene (17 mL) were added trimethyl(trimethylstannyl) stannane (3.96 g, 12.10 mmol, 2.51 mL, 3.26 eq) and Pd(PPh₃)₄ (429 mg, 0.371 mmol, 0.1 eq) under N₂, and the reaction mixture was stirred at 100° C. for 48 hours. The mixture was filtered through celite pad under vacuum and washed with MTBE (20 mL×3). The filtrate was evaporated, and the resulting residue was purified by flash chromatography on SiO₂ (gradient: 0˜7% ethyl acetate in petroleum ether) to afford (5-chloro-4-isoquinolyl)-trimethyl-stannane (817 mg, 2.18 mmol, 59% yield) as a yellow oil (combined with yellow solid. LC/MS (ESI) m/z: 328.1 [M+H]⁺.

Step 2: Preparation of tert-butyl3-[7-(5-chloro-4-isoquinolyl)-2-(2,2-dimethoxyethoxy)-8-fluoro-pyrido[4,3-d]pyrimidin-4-yl]-3,8-diazabicyclo[3.2.1]octane-8-carboxylate

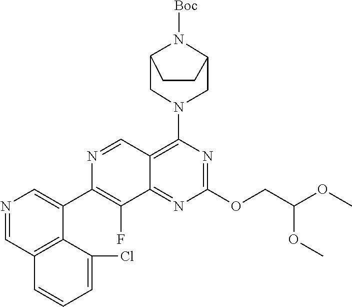 [SMILES: COC(COc1nc(N2CC3CCC(C2)N3C(=O)OC(C)(C)C)c2cnc(-c3cncc4cccc(Cl)c34)c(F)c2n1)OC]

To a solution of (5-chloro-4-isoquinolyl)-trimethyl-stannane (811 mg, 2.49 mmol, 2.25 eq) and tert-butyl 3-[7-chloro-2-(2,2-dimethoxyethoxy)-8-fluoro-pyrido[4,3-d]pyrimidin-4-yl]-3,8-diazabicyclo[3.2.1]octane-8-carboxylate (550 mg, 1.10 mmol, 1 eq) in toluene (13 mL) were added CuI (63 mg, 0.331 mmol, 0.3 eq), BINAP (138 mg, 0.221 mmol, 0.2 eq), and Pd(dppf)Cl₂ (81 mg, 0.110 mmol, 0.1 eq), and the reaction mixture was degassed and purged with nitrogen (3×), then stirred at 95° C. for 24 hours. The mixture was filtered, washing with EtOAc. The filtrate was concentrated, and the resulting residue was purified by flash chromatography on SiO₂ (gradient: 0˜6% methanol in dichloromethane) to afford tert-butyl3-[7-(5-chloro-4-isoquinolyl)-2-(2,2-dimethoxyethoxy)-8-fluoro-pyrido[4,3-d]pyrimidin-4-yl]-3,8-diazabicyclo[3.2.1]octane-8-carboxylate (499 mg, 0.599 mmol, 54% yield) as a yellow solid. LC/MS (ESI) m/z: 625.3 [M+H]⁺.

Step 3: Preparation of tert-butyl3-[2-(2,2-dimethoxyethoxy)-8-fluoro-7-(5-vinyl-4-isoquinolyl)pyrido[4,3-d]pyrimidin-4-yl]-3,8-diazabicyclo[3.2.1]octane-8-carboxylate

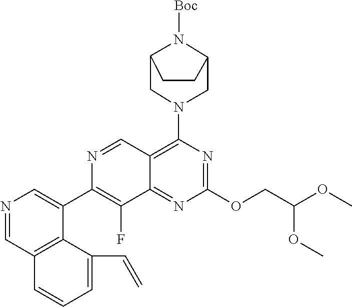 [SMILES: C=Cc1cccc2cncc(-c3ncc4c(N5CC6CCC(C5)N6C(=O)OC(C)(C)C)nc(OCC(OC)OC)nc4c3F)c12]

To a mixture of tert-butyl 3-[7-(5-chloro-4-isoquinolyl)-2-(2,2-dimethoxyethoxy)-8-fluoro-pyrido[4,3-d]pyrimidin-4-yl]-3,8-diazabicyclo[3.2.1]octane-8-carboxylate (376 mg, 0.271 mmol, 45% purity, 1 eq) and K₃PO₄ (172 mg, 0.812 mmol, 3.0 eq) in dioxane (6 mL) and H₂O (1.5 mL) were added potassium; ethenyl(trifluoro)boranuide (109 mg, 0.812 mmol, 3.0 eq) and [2-(2-aminophenyl)phenyl]palladium (1+); bis(1-adamantyl)-butyl-phosphane; methanesulfonate (30 mg, 0.040 mmol, 0.15 eq), and the reaction mixture was stirred at 95° C. for 16 hours under N₂. The mixture was partitioned between water (10 mL) and EtOAc (20 mL). The organic phase was separated, and the aqueous phase was further extracted with ethyl acetate (10 mL×3). The combined organic extract was washed with brine (20 mL), dried over anhydrous sodium sulfate, filtered, and concentrated. The resulting residue was purified by flash chromatography on SiO₂ (gradient: 0˜3% methanol in dichloromethane) to afford tert-butyl3-[2-(2,2-dimethoxyethoxy)-8-fluoro-7-(5-vinyl-4-isoquinolyl)pyrido[4,3-d]pyrimidin-4-yl]-3,8-diazabicyclo[3.2.1]octane-8-carboxylate (322 mg, crude) as a yellow solid. LC/MS (ESI) m/z: 617.2 [M+H]⁺.

Step 4: Preparation of tert-butyl3-[2-(2,2-dimethoxyethoxy)-7-(5-ethyl-4-isoquinolyl)-8-fluoro-pyrido[4,3-d]pyrimidin-4-yl]-3,8-diazabicyclo[3.2.1]octane-8-carboxylate

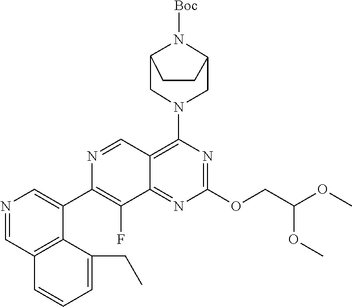 [SMILES: CCc1cccc2cncc(-c3ncc4c(N5CC6CCC(C5)N6C(=O)OC(C)(C)C)nc(OCC(OC)OC)nc4c3F)c12]

To a solution of tert-butyl 3-[2-(2,2-dimethoxyethoxy)-8-fluoro-7-(5-vinyl-4-isoquinolyl)pyrido[4,3-d]pyrimidin-4-yl]-3,8-diazabicyclo[3.2.1]octane-8-carboxylate (320 mg, 0.519 mmol, 1 eq) in EtOH (10 mL) was added dry Pd(OH)₂/C (64 mg, 0.104 mmol, 20% (wt %), 0.2 eq) under Ar, and the reaction mixture was degassed and purged with H₂ (3×), then stirred at 20° C. for 3 hours under H₂ (15 psi). The mixture was filtered through Celite pad under vacuum and rinsed with CH₃OH (20 mL×3). The filtrate was evaporated to afford tert-butyl3-[2-(2,2-dimethoxyethoxy)-7-(5-ethyl-4-isoquinolyl)-8-fluoro-pyrido[4,3-d]pyrimidin-4-yl]-3,8-diazabicyclo[3.2.1]octane-8-carboxylate (284 mg, 0.404 mmol, 78% yield) as a yellow solid. LC/MS (ESI) m/z: 619.2 [M+H]⁺.

Step 5: Preparation of tert-butyl3-[7-(5-ethyl-4-isoquinolyl)-8-fluoro-2-(2-oxoethoxy)pyrido[4,3-d]pyrimidin-4-yl]-3,8-diazabicyclo[3.2.1]octane-8-carboxylate

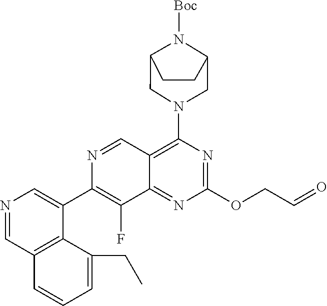 [SMILES: CCc1cccc2cncc(-c3ncc4c(N5CC6CCC(C5)N6C(=O)OC(C)(C)C)nc(OCC=O)nc4c3F)c12]

To a solution of tert-butyl 3-[2-(2,2-dimethoxyethoxy)-7-(5-ethyl-4-isoquinolyl)-8-fluoro-pyrido[4,3-d]pyrimidin-4-yl]-3,8-diazabicyclo[3.2.1]octane-8-carboxylate (284 mg, 0.459 mmol, 1 eq) in acetone (1.2 mL) was added aqueous HCl (12 M, 1.19 mL, 31 eq), and the reaction mixture was stirred at 20° C. for 10 minutes. The pH was adjusted to pH=8 by addition of NaHCO₃ (1.74 g, 20.66 mmol, 45 eq) in THF (8 mL) and H₂O (4 mL). After 10 minutes, Boc₂O (150 mg, 0.689 mmol, 1.5 eq) was added, and the reaction mixture was stirred at 20° C. for 1 hour. The mixture was extracted with ethyl acetate (30 mL×3), and the combined organic extract was washed with brine (30 mL), dried over anhydrous sodium sulfate, filtered, and concentrated. The resulting residue was purified by flash chromatography on SiO₂ (gradient: 10˜85% ethyl acetate in petroleum ether) to afford tert-butyl3-[7-(5-ethyl-4-isoquinolyl)-8-fluoro-2-(2-oxoethoxy)pyrido[4,3-d]pyrimidin-4-yl]-3,8-diazabicyclo[3.2.1]octane-8-carboxylate (270 mg, 0.472 mmol, 70% yield) as a yellow solid. LC/MS (ESI) m/z: 591.3 [M+H₂O+H]⁺.

Step 6: Preparation of (2S,4R)-1-[(2R)-2-[3-[[1-[2-[[4-(3,8-diazabicyclo[3.2.1]octan-3-yl)-7-(5-ethyl-4-isoquinolyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

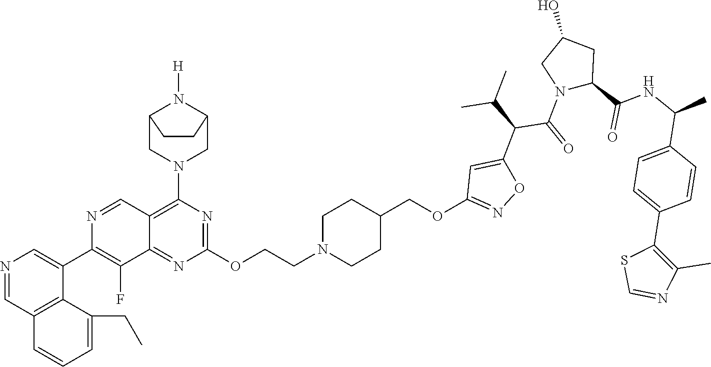 [SMILES: CCc1cccc2cncc(-c3ncc4c(N5CC6CCC(C5)N6)nc(OCCN5CCC(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

The title compound was made in an analogous manner to (2S,4R)-1-[(2R)-2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(5-ethyl-4-isoquinolyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide. (formic acid salt, yellow solid). LC/MS (ESI) m/z: 1052.9 [M+H]⁺. ¹H NMR (400 MHz, CD₃OD) δ 9.37 (s, 1H), 9.15 (s, 1H), 8.87 (s, 1H), 8.50 (s, 1H), 8.34 (s, 1H), 8.18-8.06 (m, 1H), 7.79-7.68 (m, 2H), 7.48-7.34 (m, 4H), 6.04-5.90 (m, 1H), 5.08-4.98 (m, 1H), 5.09-5.00 (m, 2H), 4.70-4.65 (m, 1H), 4.55-4.48 (m, 1H), 4.42-4.41 (m, 1H), 4.12-4.03 (m, 2H), 3.95-3.71 (m, 5H), 3.70-3.58 (m, 2H), 3.55-3.35 (m, 3H), 3.25-3.14 (m, 2H), 2.70-2.55 (m, 2H), 2.54-2.28 (m, 6H), 2.24-2.13 (m, 1H), 2.09-1.86 (m, 8H), 1.64-1.46 (m, 5H), 1.05 (d, J=6.4 Hz, 3H), 0.98-0.85 (m, 6H).

Exemplary Synthesis of (2S,4R)-1-[(2R)-2-[3-[[3-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 288)

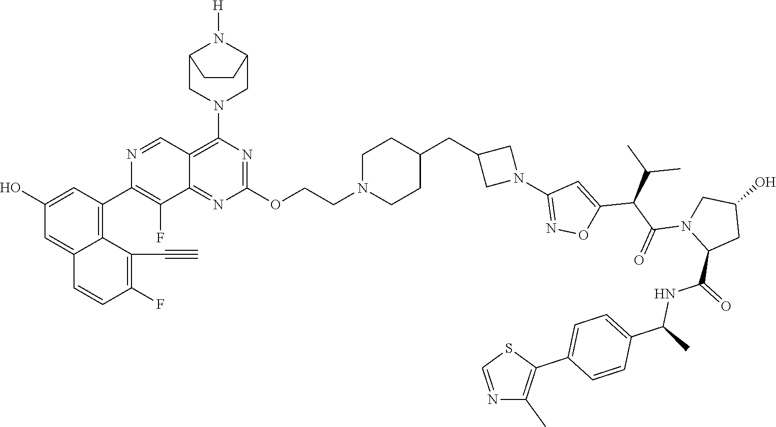 [SMILES: C#Cc1c(F)ccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was made in an analogous manner to (2S,4R)-1-((R)-2-(3-(4-((1-(2-((4-((1R,5S)-3,8-diazabicyclo[3.2.1]octan-3-yl)-7-(8-ethynyl-7-fluoro-3-hydroxynaphthalen-1-pyrimidin-2-yl)oxy)ethyl) piperidin-4-yl)methyl) piperidin-1-yl)-8-fluoropyrido[4,3-d]yl)isoxazol-5-yl)-3-methylbutanoyl)-4-hydroxy-N—((S)-1-(4-(4-methylthiazol-5-yl)phenyl)ethyl)pyrrolidine-2-carboxamide starting from tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-[[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide. (yellow solid). LC/MS (ESI) m/z: 1120.9. [M+H]⁺. ¹H NMR (400 MHz, CD₃OD) δ 9.01 (s, 1H), 8.87 (s, 1H), 7.87-7.84 (m, 1H), 7.48-7.30 (m, 6H), 7.21 (d, J=2.4 Hz, 1H), 5.83-5.79 (m, 1H), 5.03 (q, J=6.8 Hz, 1H), 4.50-4.35 (m, 2H), 4.04 (t, J=7.6 Hz, 2H), 3.85-3.81 (m, 1H), 3.74-3.53 (m, 9H), 3.13-3.10 (m, 2H), 2.92-2.87 (m, 3H), 2.48 (s, 3H), 2.40-2.28 (m, 1H), 2.21-2.16 (m, 3H), 1.99-1.77 (m, 6H), 1.70-1.57 (m, 5H), 1.52 (d, J=6.8 Hz, 3H), 1.36-1.26 (M, 5H), 1.04 (d, J=6.4 Hz, 3H), 0.91-0.87 (m, 3H).

Exemplary Synthesis of (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 275)

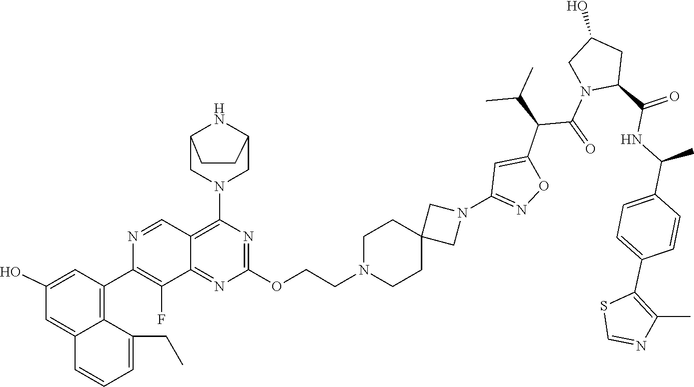 [SMILES: CCc1cccc2cc(O)cc(-c3ncc4c(N5CC6CCC(C5)N6)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12]

The title compound was made in an analogous manner to (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-(azetidin-3-yl)piperidine-1-carboxylate. (white solid). LC/MS (ESI) m/z: 1092.7 [M+H]⁺. ¹H NMR (400 MHz, CD₃OD) δ 9.04 (s, 1H), 8.88 (s, 1H), 7.69-7.58 (m, 1H), 7.51-7.42 (m, 3H), 7.41-7.32 (m, 2H), 7.28 (d, J=2.4 Hz, 1H), 7.16 (d, J=7.2 Hz, 1H), 7.06-6.95 (m, 1H), 5.90-5.77 (m, 1H), 4.83-4.72 (m, 1H), 4.66-4.58 (m, 6H), 4.50 (t, J=8.0 Hz, 1H), 4.43 (br s, 1H), 4.03-3.94 (m, 2H), 3.88-3.79 (m, 1H), 3.77-3.69 (m, 2H), 3.69-3.64 (m, 4H), 3.62-3.56 (m, 1H), 3.12 (br d, J=11.3 Hz, 2H), 2.92-2.83 (m, 2H), 2.60-2.51 (m, 1H), 2.48 (s, 3H), 2.40-2.25 (m, 3H), 2.23-2.14 (m, 3H), 2.05-1.92 (m, 1H), 1.91-1.83 (m, 2H), 1.83-1.77 (m, 2H), 1.76-1.67 (m, 2H), 1.60-1.50 (m, 4H), 1.33-1.20 (m, 2H), 1.04 (d, J=6.4 Hz, 3H), 0.93-0.83 (m, 6H).

Exemplary Synthesis of (2S,4R)-1-[(2S)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 277)

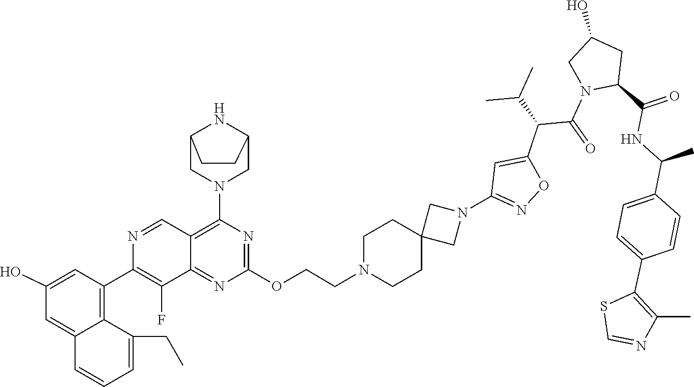 [SMILES: CCc1cccc2cc(O)cc(-c3ncc4c(N5CC6CCC(C5)N6)nc(OCCN5CCC6(CC5)CN(c5cc([C@@H](C(=O)N7C[C@H](O)C[C@H]7C(=O)N[C@@H](C)c7ccc(-c8scnc8C)cc7)C(C)C)on5)C6)nc4c3F)c12]

The title compound was made in an analogous manner to (2S,4R)-1-[(2S)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-(azetidin-3-yl)piperidine-1-carboxylate. (white solid). LC/MS (ESI) m/z: 1093.8 [M+H]⁺. ¹H NMR (400 MHz, CD₃OD) δ 9.04 (s, 1H), 8.94-8.82 (m, 1H), 7.62 (d, J=8.0 Hz, 1H), 7.50-7.42 (m, 1H), 7.41 (s, 1H), 7.38-7.32 (m, 3H), 7.28 (d, J=2.4 Hz, 1H), 7.18-7.11 (m, 1H), 7.01 (d, J=2.0 Hz, 1H), 5.91-5.79 (m, 1H), 4.66-4.56 (m, 8H), 4.43 (br s, 1H), 4.00-3.89 (m, 2H), 3.75-3.69 (m, 3H), 3.69-3.61 (m, 6H), 3.16-3.06 (m, 2H), 2.93-2.81 (m, 2H), 2.53-2.48 (m, 1H), 2.47 (s, 3H), 2.41-2.27 (m, 3H), 2.25-2.15 (m, 3H), 1.96 (ddd, J=4.8, 8.4, 13.2 Hz, 1H), 1.90-1.74 (m, 4H), 1.68 (br d, J=12.4 Hz, 2H), 1.58 (d, J=7.2 Hz, 1H), 1.49 (d, J=7.2 Hz, 3H), 1.28-1.13 (m, 2H), 1.04 (d, J=6.8 Hz, 3H), 0.94-0.87 (m, 6H).

Exemplary Synthesis of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 262)

Step 1: Preparation of tert-butyl 4-fluoro-4-formyl-piperidine-1-carboxylate

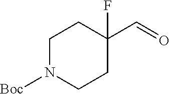 [SMILES: CC(C)(C)OC(=O)N1CCC(F)(C=O)CC1]

To a solution of DMSO (502 mg, 6.43 mmol, 3.0 eq) in anhydrous CH₂Cl₂ (5.0 mL) at −78° C. under N₂ was added a solution of (COCl)₂ (598 mg, 4.72 mmol, 2.2 eq) in anhydrous CH₂Cl₂ (2.0 mL) dropwise, and the resulting mixture was stirred at −78° C. for 20 minutes. A solution of tert-butyl 4-fluoro-4-(hydroxymethyl)piperidine-1-carboxylate (500 mg, 2.14 mmol, 1.0 eq) in anhydrous CH₂Cl₂ (5.0 mL) was then added dropwise, and the resulting mixture was stirred at −78° C. for 30 minutes. Triethylamine (2.17 g, 21.43 mmol, 10.0 eq). was then added dropwise, and the reaction mixture was stirred at −78° C. for 10 minutes. The mixture was diluted with water (10 mL) and extracted with dichloromethane (40 mL×3). The combined organic extract was washed with brine (15 mL), dried over anhydrous sodium sulfate, filtered, and concentrated to give tert-butyl 4-fluoro-4-formyl-piperidine-1-carboxylate (550 mg, crude) as a yellow oil. ¹H NMR (400 MHz, CDCl₃) δ 9.66 (d, J=5.2 Hz, 1H), 4.06-3.96 (m, 2H), 3.05-2.92 (m, 2H), 1.84-1.71 (m, 4H), 1.39 (s, 9H).

Step 2: Preparation of tert-butyl 4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate

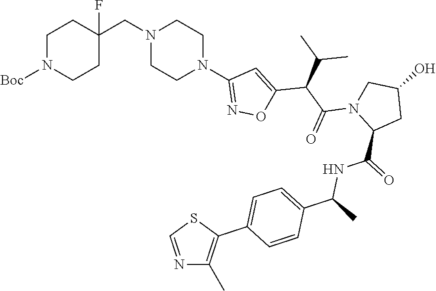 [SMILES: Cc1ncsc1-c1ccc([C@H](C)NC(=O)[C@@H]2C[C@@H](O)CN2C(=O)[C@@H](c2cc(N3CCN(CC4(F)CCN(C(=O)OC(C)(C)C)CC4)CC3)no2)C(C)C)cc1]

To a solution of tert-butyl 4-fluoro-4-formyl-piperidine-1-carboxylate (979 mg, 3.81 mmol theoretically, 7.2 eq) and (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide (300 mg, 0.529 mmol, 1.0 eq) in CH₂Cl₂ (20 mL) were added HOAc (159 mg, 2.65 mmol, 5.0 eq) and NaBH(OAc)₃ (561 mg, 2.65 mmol, 5.0 eq), and the reaction mixture was stirred at 25° C. for 15 hours. The mixture was concentrated, and the resulting residue was purified by flash chromatography on SiO₂ (gradient: 0˜7% CH₃OH in CH₂Cl₂) to afford tert-butyl 4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (260 mg, 0.293 mmol, 55% yield) as a light yellow solid. LC/MS (ESI) m/z: 782.5. [M+H]⁺.

Step 3: Preparation of (2S,4R)-1-[(2R)-2-[3-[4-[(4-fluoro-4-piperidyl)methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

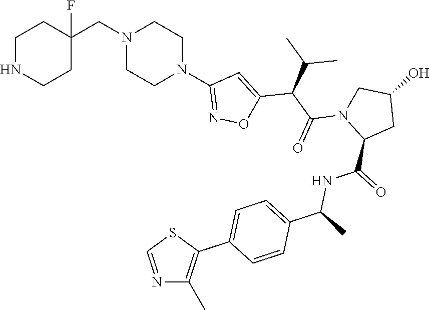 [SMILES: Cc1ncsc1-c1ccc([C@H](C)NC(=O)[C@@H]2C[C@@H](O)CN2C(=O)[C@@H](c2cc(N3CCN(CC4(F)CCNCC4)CC3)no2)C(C)C)cc1]

To a solution of tert-butyl 4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (260 mg, 88% purity, 0.293 mmol) in CH₂Cl₂ (5.0 mL) was added 4 M HCl/dioxane (3.0 mL), and the reaction mixture was stirred at 25° C. for 30 minutes. Petroleum ether (50 mL) was added, and the resulting precipitate was treated with saturated aqueous NaHCO₃ solution to pH ˜8. The resulting mixture was extracted with CH₂Cl₂ (30 mL×2), and the combined organic extracts were washed with brine (30 mL), dried over anhydrous Na₂SO₄, filtered, and concentrated to afford (2S,4R)-1-[(2R)-2-[3-[4-[(4-fluoro-4-piperidyl)methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (240 mg, crude) as a light yellow solid. LC/MS (ESI) m/z: 682.3. [M+H]⁺.

Step 4: Preparation of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

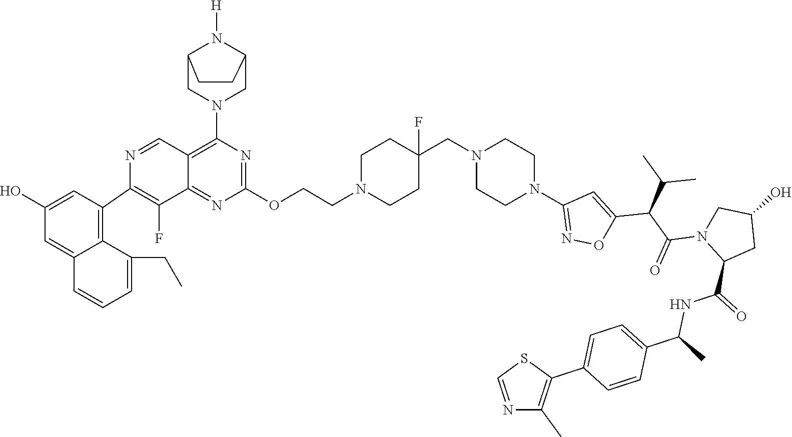 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-1-[(2R)-2-[3-[4-[(4-fluoro-4-piperidyl)methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). LC/MS (ESI) m/z: 1154.0. [M+H]⁺. ¹H NMR (400 MHz, CD₃OD) δ 9.04 (s, 1H), 8.87 (s, 1H), 7.62 (d, J=8.0 Hz, 1H), 7.46-7.34 (m, 5H), 7.28 (d, J=2.8 Hz, 1H), 7.16 (d, J=7.6 Hz, 1H), 7.01 (d, J=2.8 Hz, 1H), 6.09 (s, 1H), 5.49 (s, 2H), 5.11-4.97 (m, 1H), 4.66-4.60 (m, 6H), 4.51 (t, J=8.0 Hz, 1H), 4.44 (s, 1H), 3.86-3.82 (m, 1H), 3.75-3.69 (m, 4H), 3.64-3.56 (m, 2H), 3.27-3.18 (m, 4H), 2.95-2.84 (m, 4H), 2.63 (t, J=4.8 Hz, 4H), 2.59-2.50 (m, 3H), 2.48 (s, 3H), 2.36-2.22 (m, 3H), 1.97-1.80 (m, 7H), 1.52 (d, J=7.2 Hz, 3H), 1.04 (d, J=6.4 Hz, 3H), 0.92-0.87 (m, 6H).

Exemplary Synthesis of (2S,4R)-1-[(2R)-2-[3-[2-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 249)

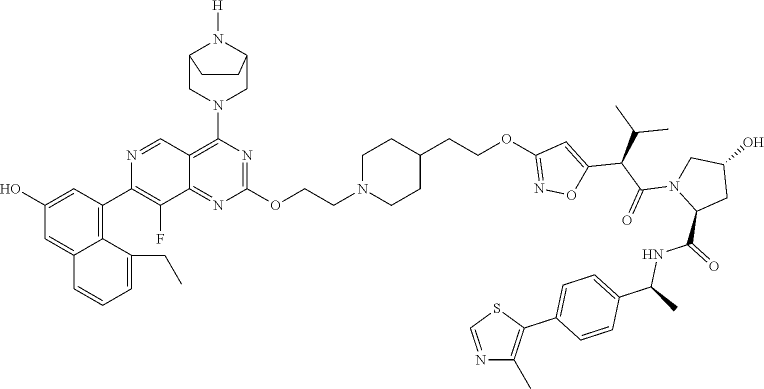 [SMILES: CCc1cccc2cc(O)cc(-c3ncc4c(N5CC6CCC(C5)N6)nc(OCCN5CCC(CCOc6cc([C@H](C(=O)N7C[C@H](O)C[C@H]7C(=O)N[C@@H](C)c7ccc(-c8scnc8C)cc7)C(C)C)on6)CC5)nc4c3F)c12]

The title compound was made in an analogous manner to (2S,4R)-1-[(2R)-2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-(2-hydroxyethyl)piperidine-1-carboxylate. (formic acid salt, white solid). LC/MS (ESI) m/z: 1081.9 [M+H]⁺. ¹H NMR (400 MHz, CD₃OD) δ 9.10 (s, 1H), 8.89-8.83 (m, 1H), 8.50 (s, 1H), 7.63 (d, J=8.0 Hz, 1H), 7.46-7.33 (m, 5H), 7.30 (d, J=2.4 Hz, 1H), 7.16 (d, J=6.8 Hz, 1H), 7.01 (d, J=2.4 Hz, 1H), 6.00-5.90 (m, 1H), 5.06-4.97 (m, 1H), 4.80-4.75 (m, 3H), 4.74-4.64 (m, 2H), 4.51 (t, J=8.4 Hz, 1H), 4.46-4.37 (m, 1H), 4.25 (t, J=5.6 Hz, 2H), 3.95 (d, J=6.8 Hz, 2H), 3.88-3.80 (m, 2H), 3.70-3.64 (m, 1H), 3.60 (d, J=10.8 Hz, 1H), 3.54-3.48 (m, 2H), 3.36 (d, J=4.4 Hz, 2H), 2.89-2.74 (m, 2H), 2.51-2.42 (m, 3H), 2.42-2.14 (m, 4H), 2.08-1.89 (m, 7H), 1.76 (s, 3H), 1.60-1.45 (m, 5H), 1.05 (d, J=6.4 Hz, 3H), 0.93-0.85 (m, 6H).

Exemplary Synthesis of (2S,4R)-1-[(2S)-2-[3-[2-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 248)

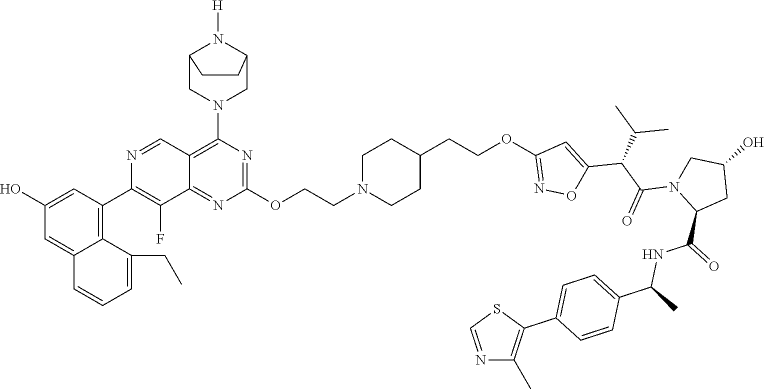 [SMILES: CCc1cccc2cc(O)cc(-c3ncc4c(N5CC6CCC(C5)N6)nc(OCCN5CCC(CCOc6cc([C@@H](C(=O)N7C[C@H](O)C[C@H]7C(=O)N[C@@H](C)c7ccc(-c8scnc8C)cc7)C(C)C)on6)CC5)nc4c3F)c12]

The title compound was made in an analogous manner to (2S,4R)-1-[(2S)-2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-(2-hydroxyethyl)piperidine-1-carboxylate. (formic acid salt, white solid). LC/MS (ESI) m/z: 1081.9 [M+H]⁺. ¹H NMR (400 MHz, CD₃OD) δ 9.09 (s, 1H), 8.918.83 (m, 1H), 8.51 (s, 1H), 7.63 (d, J=7.6 Hz, 1H), 7.497.34 (m, 5H), 7.30 (d, J=2.6 Hz, 1H), 7.16 (d, J=6.8 Hz, 1H), 7.01 (d, J=2.0 Hz, 1H), 6.03-5.94 (m, 1H), 4.99 (q, J=7.0 Hz, 1H), 4.79-4.73 (m, 3H), 4.68 (d, J=14.8 Hz, 2H), 4.57 (t, J=8.0 Hz, 1H), 4.43 (s, 1H), 4.29-4.18 (m, 2H), 3.90 (d, J=2.0 Hz, 2H), 3.83 (d, J=15.0 Hz, 2H), 3.79-3.72 (m, 2H), 3.72-3.62 (m, 2H), 3.46 (d, J=11.2 Hz, 2H), 2.73 (t, J=12.0 Hz, 2H), 2.50-2.44 (m, 3H), 2.40-2.32 (m, 2H), 2.32-2.19 (m, 2H), 2.00-1.87 (m, 7H), 1.77-1.68 (m, 3H), 1.59-1.44 (m, 5H), 1.07-0.94 (m, 3H), 0.92-0.83 (m, 6H).

Exemplary Synthesis of (2S,4R)-1-[(2R)-2-[3-[[1-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-3-yl]oxyisoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 344)

Step 1: Preparation of (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide

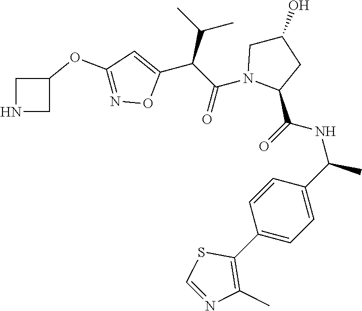 [SMILES: Cc1ncsc1-c1ccc([C@H](C)NC(=O)[C@@H]2C[C@@H](O)CN2C(=O)[C@@H](c2cc(OC3CNC3)no2)C(C)C)cc1]

The title compound was made in an analogous manner to (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide starting from tert-butyl 3-hydroxyazetidine-1-carboxylate. (white solid). LC/MS (ESI) m/z: 554.2 [M+H]⁺.

Step 2: Preparation of tert-butyl 4-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidin-1-yl]methyl]piperidine-1-carboxylate

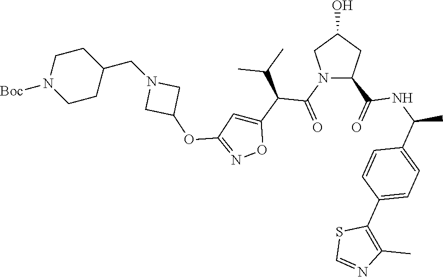 [SMILES: Cc1ncsc1-c1ccc([C@H](C)NC(=O)[C@@H]2C[C@@H](O)CN2C(=O)[C@@H](c2cc(OC3CN(CC4CCN(C(=O)OC(C)(C)C)CC4)C3)no2)C(C)C)cc1]

To a solution of tert-butyl 4-formylpiperidine-1-carboxylate (220 mg, 1.03 mmol, 1.5 eq) and (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide (380 mg, 0.686 mmol, 1.0 eq) in isopropanol (1.0 mL) and CH₂Cl₂ (5.0 mL) were added 2-methylpyridine borane (367 mg, 3.43 mmol, 5.0 eq) and AcOH (196 uL, 3.43 mmol, 5.0 eq), and the reaction mixture was stirred at 25° C. for 1 hour. The reaction mixture was concentrated, and the resulting residue was purified by flash chromatography on SiO₂ (gradient: 0˜9% methanol in dichloromethane) to afford tert-butyl 4-[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidin-1-yl]methyl]piperidine-1-carboxylate (365 mg, 0.486 mmol, 66% yield) as a white solid. LC/MS (ESI) m/z: 751.4 [M+H]⁺.

Step 4: Preparation of (2S,4R)-4-hydroxy-1-[(2R)-3-methyl-2-[3-[[1-(4-piperidylmethyl) azetidin-3-yl]oxyisoxazol-5-yl]butanoyl]-N-[(1S)-1-[4-(4-methylthiazol-5-yl)phenyl]ethyl]pyrrolidine-2-carboxamide

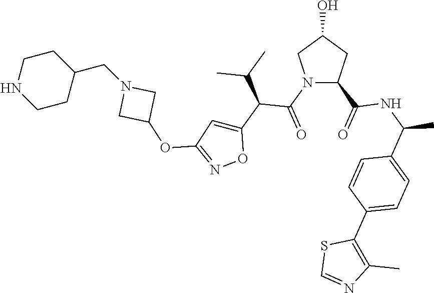 [SMILES: Cc1ncsc1-c1ccc([C@H](C)NC(=O)[C@@H]2C[C@@H](O)CN2C(=O)[C@@H](c2cc(OC3CN(CC4CCNCC4)C3)no2)C(C)C)cc1]

To a solution of tert-butyl 4-[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidin-1-yl]methyl]piperidine-1-carboxylate (360 mg, 0.479 mmol, 1.0 eq) in CH₂Cl₂ (3.0 mL) was added HCl/dioxane (3 mL, 4 M, 25 eq), and the reaction mixture was stirred at 25° C. for 30 minutes. The mixture was diluted with petroleum ether (50 mL) and filtered. The filter cake was dissolved in tetrahydrofuran (30 mL) and treated with triethylamine (3.0 mL). The mixture was filtered and concentrated to give (2S,4R)-4-hydroxy-1-[(2R)-3-methyl-2-[3-[[1-(4-piperidylmethyl) azetidin-3-yl]oxyisoxazol-5-yl]butanoyl]-N-[(1S)-1-[4-(4-methylthiazol-5-yl)phenyl]ethyl]pyrrolidine-2-carboxamide (260 mg, 0.400 mmol, 73% yield) as a white solid. LC/MS (ESI) m/z: 651.2 [M+H]⁺.

Step 5: Preparation of (2S,4R)-1-[(2R)-2-[3-[[1-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-3-yl]oxyisoxazol-5-yl]-3-methyl-butanoyl]-4-hydroxy-N-[(1S)-1-[4-(4-methylthiazol-5-yl)phenyl]ethyl]pyrrolidine-2-carboxamide

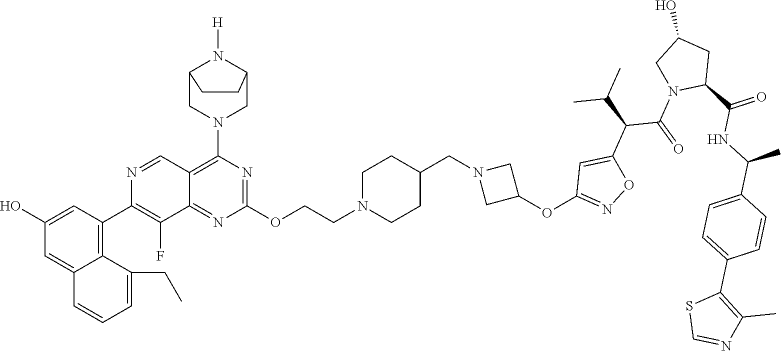 [SMILES: CCc1cccc2cc(O)cc(-c3ncc4c(N5CC6CCC(C5)N6)nc(OCCN5CCC(CN6CC(Oc7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was made in an analogous manner to (2S,4R)-1-[(2R)-2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-4-hydroxy-1-[(2R)-3-methyl-2-[3-[[1-(4-piperidylmethyl) azetidin-3-yl]oxyisoxazol-5-yl]butanoyl]-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate. (formic acid salt, white solid). LC/MS (ESI) m/z: 1122.9 [M+H]⁺. ¹H NMR (400 MHz, CD₃OD) δ 9.10 (s, 1H), 8.88 (s, 1H), 7.64 (d, J=8.4 Hz, 1H), 7.46-7.35 (m, 5H), 7.30 (d, J=2.4 Hz, 1H), 7.17 (d, J=7.2 Hz, 1H), 7.00 (d, J=2.4 Hz, 1H), 6.02 (s, 1H), 5.07-4.97 (m, 3H), 4.79-4.77 (m, 3H), 4.50 (t, J=8.4 Hz, 1H), 4.44 (s, 1H), 4.08-4.00 (m, 2H), 3.93-3.82 (m, 4H), 3.69 (d, J=10.0 Hz, 1H), 3.61 (d, J=11.2 Hz, 1H), 3.49-3.45 (m, 2H), 3.38-3.34 (m, 1H), 2.79 (t, J=11.2 Hz, 2H), 2.62 (d, J=7.2 Hz, 2H), 2.48 (s, 3H), 2.42-2.13 (m, 5H), 2.10-1.84 (m, 8H), 1.58 (d, J=7.2 Hz, 2H), 1.55-1.35 (m, 5H), 1.05 (d, J=6.5 Hz, 3H), 0.94-0.83 (m, 7H).

Exemplary Synthesis of (2S,4R)-1-[(2S)-2-[3-[[1-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-3-yl]oxyisoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 245)

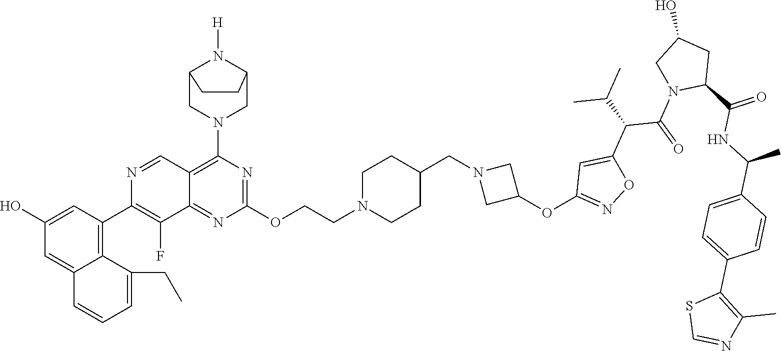 [SMILES: CCc1cccc2cc(O)cc(-c3ncc4c(N5CC6CCC(C5)N6)nc(OCCN5CCC(CN6CC(Oc7cc([C@@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was made in an analogous manner to (2S,4R)-1-[(2R)-2-[3-[[1-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-3-yl]oxyisoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-4-hydroxy-1-[(2S)-3-methyl-2-[3-[[1-(4-piperidylmethyl) azetidin-3-yl]oxyisoxazol-5-yl]butanoyl]-N-[(1S)-1-[4-(4-methylthiazol-5-yl)phenyl]ethyl]pyrrolidine-2-carboxamide (200 mg, 0.307 mmol, 1.0 eq) and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate. (formic acid salt, white solid). LC/MS (ESI) m/z: 1122.2 [M+H]⁺. ¹H NMR (400 MHz, CD₃OD) δ 9.09 (s, 1H), 8.87 (s, 1H), 7.63 (d, J=8.4 Hz, 1H), 7.47-7.28 (m, 6H), 7.17 (d, J=7.2 Hz, 1H), 7.00 (d, J=2.4 Hz, 1H), 6.03 (s, 1H), 5.02-4.97 (m, 3H), 4.77 (s, 3H), 4.59-4.55 (m, 1H), 4.43 (s, 1H), 3.99-3.93 (m, 2H), 3.89-3.83 (m, 3H), 3.77 (d, J=9.2 Hz, 1H), 3.71-3.62 (m, 2H), 3.47 (d, J=11.2 Hz, 2H), 3.38-3.37 (m, 1H), 2.73 (t, J=12.0 Hz, 2H), 2.56 (d, J=6.8 Hz, 2H), 2.47 (s, 3H), 2.42-2.18 (m, 5H), 2.07-1.84 (m, 8H), 1.70-1.55 (m, 2H), 1.52-1.37 (m, 5H), 1.05 (d, J=6.4 Hz, 3H), 0.96-0.82 (m, 7H).

Exemplary Synthesis of (2S,4R)-1-[(2R)-2-[3-[2-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-methyl-4-piperidyl]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 237)

Step 1: (2S,4R)-4-hydroxy-1-[(2R)-3-methyl-2-[3-[2-(4-methyl-4-piperidyl) ethoxy]isoxazol-5-yl]butanoyl]-N-[(1S)-1-[4-(4-methylthiazol-5-yl)phenyl]ethyl]pyrrolidine-2-carboxamide

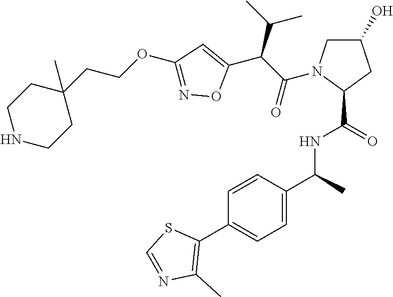 [SMILES: Cc1ncsc1-c1ccc([C@H](C)NC(=O)[C@@H]2C[C@@H](O)CN2C(=O)[C@@H](c2cc(OCCC3(C)CCNCC3)no2)C(C)C)cc1]

The title compound was made in an analogous manner to (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide starting from tert-butyl 4-(2-hydroxyethyl)-4-methyl-piperidine-1-carboxylate. (yellow solid). LC/MS (ESI) m/z: 624.0 [M+H]⁺.

Step 2: Preparation of WS-ARV-JM-099-A-E2, (2S,4R)-1-[(2R)-2-[3-[2-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-methyl-4-piperidyl]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

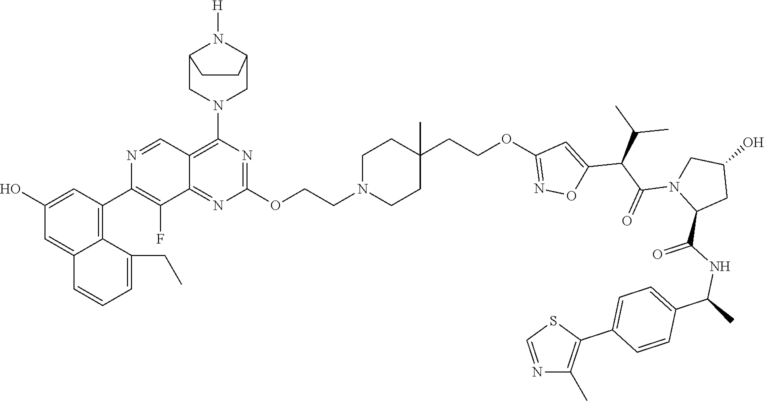 [SMILES: CCc1cccc2cc(O)cc(-c3ncc4c(N5CC6CCC(C5)N6)nc(OCCN5CCC(C)(CCOc6cc([C@H](C(=O)N7C[C@H](O)C[C@H]7C(=O)N[C@@H](C)c7ccc(-c8scnc8C)cc7)C(C)C)on6)CC5)nc4c3F)c12]

The title compound was made in an analogous manner to (2S,4R)-1-[(2R)-2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-[2-(4-methyl-4-piperidyl) ethoxy]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1095.9 [M+H]⁺. ¹H NMR (400 MHz, CD₃OD) δ 9.08 (s, 1H), 8.90-8.81 (m, 1H), 8.48 (s, 1H), 7.63 (d, J=8.4 Hz, 1H), 7.47-7.32 (m, 5H), 7.29 (d, J=2.4 Hz, 1H), 7.16 (d, J=6.8 Hz, 1H), 7.01 (t, J=2.0 Hz, 1H), 6.05-5.89 (m, 1H), 4.98 (d, J=7.2 Hz, 1H), 4.77 (d, J=4.4 Hz, 2H), 4.72-4.54 (m, 5H), 4.42 (d, J=2.4 Hz, 1H), 4.31-4.21 (m, 2H), 3.86 (s, 2H), 3.78 (d, J=7.4 Hz, 2H), 3.73-3.61 (m, 2H), 3.35 (s, 2H), 3.23-3.03 (m, 4H), 2.49-2.43 (m, 3H), 2.40-2.16 (m, 4H), 2.01-1.87 (m, 5H), 1.82 (t, J=6.4 Hz, 2H), 1.76-1.67 (m, 2H), 1.66-1.59 (m, 2H), 1.47 (d, J=7.2 Hz, 2H), 1.11-1.01 (m, 6H), 0.94-0.86 (m, 6H).

Exemplary Synthesis of (2S,4R)-1-[(2S)-2-[3-[2-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-methyl-4-piperidyl]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 238)

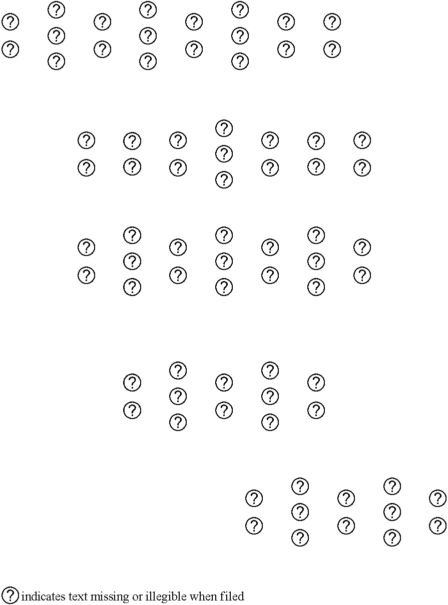

The title compound was made in an analogous manner to (2S,4R)-1-[(2R)-2-[3-[2-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-methyl-4-piperidyl]ethoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[2-(4-methyl-4-piperidyl) ethoxy]isoxazol-5-yl]butanoyl]-N-[(1S)-1-[4-(4-methylthiazol-5-yl)phenyl]ethyl]pyrrolidine-2-carboxamide. (formic acid salt, white solid). LC/MS (ESI) m/z: 1095.9 [M+H]⁺. ¹H NMR (400 MHz, CD₃OD) δ 9.12-9.06 (m, 1H), 8.91-8.84 (m, 1H), 8.49 (s, 1H), 7.63 (d, J=8.4 Hz, 1H), 7.46-7.33 (m, 5H), 7.30 (d, J=2.4 Hz, 1H), 7.17 (d, J=7.2 Hz, 1H), 7.01 (d, J=2.4 Hz, 1H), 6.00-5.89 (m, 1H), 5.04-5.00 (m, 1H), 4.73 (d, J=4.4 Hz, 1H), 4.70-4.57 (m, 5H), 4.50 (t, J=8.4 Hz, 1H), 4.45-4.37 (m, 1H), 4.30 (t, J=6.4 Hz, 2H), 3.88-3.74 (m, 5H), 3.67 (d, J=10.0 Hz, 1H), 3.61 (d, J=10.4 Hz, 1H), 3.54-3.43 (m, 1H), 3.20-3.00 (m, 4H), 2.49-2.45 (m, 3H), 2.40-2.17 (m, 4H), 1.99-1.82 (m, 7H), 1.79-1.70 (m, 2H), 1.68-1.60 (m, 2H), 1.51 (d, J=7.2 Hz, 2H), 1.11-1.07 (m, 3H), 1.04 (d, J=6.4 Hz, 3H), 0.93-0.85 (m, 6H).

Exemplary Synthesis of (2S,4R)-1-[(2R)-2-[3-[[7-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-7-azaspiro[3.5]nonan-2-yl]oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 231)

Step 1: Preparation of (2S,4R)-1-[(2R)-2-[3-(7-azaspiro[3.5]nonan-2-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide

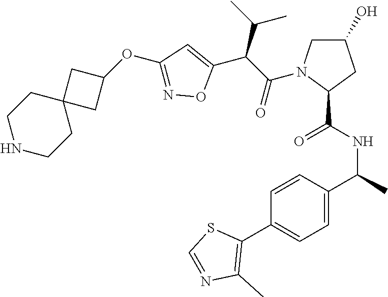 [SMILES: Cc1ncsc1-c1ccc([C@H](C)NC(=O)[C@@H]2C[C@@H](O)CN2C(=O)[C@@H](c2cc(OC3CC4(CCNCC4)C3)no2)C(C)C)cc1]

The title compound was made in an analogous manner to (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide starting from tert-butyl 2-hydroxy-7-azaspiro[3.5]nonane-7-carboxylate. (HCl salt, yellow solid). LC/MS (ESI) m/z: 622.3 [M+H]⁺.

Step 2: Preparation of (2S,4R)-1-[(2R)-2-[3-[[7-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-7-azaspiro[3.5]nonan-2-yl]oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

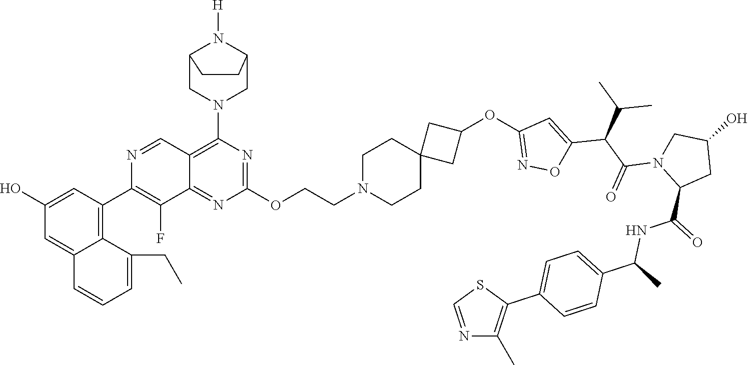 [SMILES: CCc1cccc2cc(O)cc(-c3ncc4c(N5CC6CCC(C5)N6)nc(OCCN5CCC6(CC5)CC(Oc5cc([C@H](C(=O)N7C[C@H](O)C[C@H]7C(=O)N[C@@H](C)c7ccc(-c8scnc8C)cc7)C(C)C)on5)C6)nc4c3F)c12]

The title compound was made in an analogous manner to (2S,4R)-1-[(2R)-2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-1-[(2R)-2-[3-(7-azaspiro[3.5]nonan-2-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate. (formic acid salt, white solid). LC/MS (ESI) m/z: 1093.9 [M+H]⁺. ¹H NMR (400 MHz, CD₃OD) δ 9.07 (s, 1H), 8.87 (s, 1H), 8.57-8.37 (m, 1H), 7.63 (d, J=8.0 Hz, 1H), 7.50-7.32 (m, 5H), 7.30 (s, 1H), 7.16 (d, J=7.2 Hz, 1H), 7.00 (s, 1H), 6.05-5.92 (m, 1H), 5.03-4.96 (m, 1H), 4.79-4.66 (m, 5H), 4.60-4.54 (m, 1H), 4.46-4.35 (m, 1H), 4.04-3.56 (m, 7H), 3.30-3.20 (m, 2H), 3.16-2.79 (m, 4H), 2.52-2.17 (m, 9H), 2.10-1.88 (m, 7H), 1.87-1.72 (m, 4H), 1.62-1.48 (m, 3H), 1.05 (d, J=6.4 Hz, 1H), 0.98-0.82 (m, 6H).

Exemplary Synthesis of (2S,4R)-1-[(2S)-2-[3-[[7-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-7-azaspiro[3.5]nonan-2-yl]oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 232)

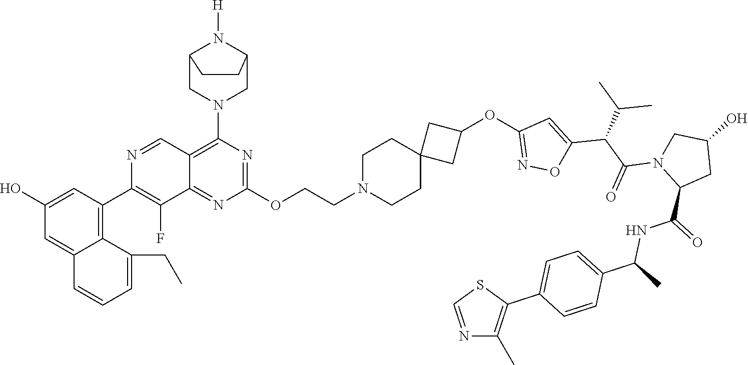 [SMILES: CCc1cccc2cc(O)cc(-c3ncc4c(N5CC6CCC(C5)N6)nc(OCCN5CCC6(CC5)CC(Oc5cc([C@@H](C(=O)N7C[C@H](O)C[C@H]7C(=O)N[C@@H](C)c7ccc(-c8scnc8C)cc7)C(C)C)on5)C6)nc4c3F)c12]

The title compound was made in an analogous manner to (2S,4R)-1-[(2R)-2-[3-[7-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-7-azaspiro[3.5]nonan-2-yl]oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-1-[(2S)-2-[3-(7-azaspiro[3.5]nonan-2-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate. (formic acid salt, white solid). LC/MS (ESI) m/z: 1093.9 [M+H]⁺. ¹H NMR (400 MHz, CD₃OD) δ 9.07 (s, 1H), 8.87 (s, 1H), 8.57-8.37 (m, 1H), 7.64 (d, J=8.0 Hz, 1H), 7.50-7.32 (m, 5H), 7.30 (d, J=2.4 Hz, 1H), 7.16 (d, J=7.2 Hz, 1H), 7.00 (d, J=2.4 Hz, 1H), 6.05-5.92 (m, 1H), 5.03-4.96 (m, 1H), 4.79-4.66 (m, 5H), 4.60-4.54 (m, 1H), 4.46-4.35 (m, 1H), 4.04-3.56 (m, 7H), 3.30-3.20 (m, 2H), 3.16-2.79 (m, 4H), 2.52-2.17 (m, 9H), 2.10-1.88 (m, 7H), 1.87-1.72 (m, 4H), 1.48 (d, J=6.8 Hz, 3H), 1.06 (d, J=6.4 Hz, 1H), 0.98-0.82 (m, 6H).

Exemplary Synthesis of (2S,4R)-1-[(2R)-2-[3-[4-[[(3S)-1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-3-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 228)

Step 1: Preparation of tert-butyl(3S)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate

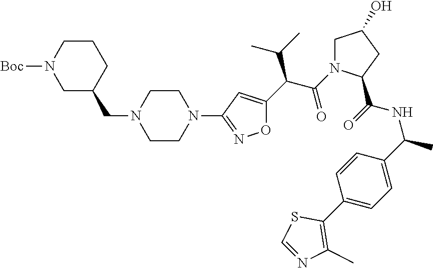 [SMILES: Cc1ncsc1-c1ccc([C@H](C)NC(=O)[C@@H]2C[C@@H](O)CN2C(=O)[C@@H](c2cc(N3CCN(C[C@@H]4CCCN(C(=O)OC(C)(C)C)C4)CC3)no2)C(C)C)cc1]

To a mixture of (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide (400 mg, 0.706 mmol, 1.0 eq) and tert-butyl(3R)-3-formylpiperidine-1-carboxylate (301 mg, 1.41 mmol, 2.0 eq) in CH₂Cl₂ (6 mL) and isopropanol (6 mL) were added AcOH (161 uL,d=1.05 g/mL, 2.82 mmol, 4.0 eq) and 2-methylpyridine borane (377 mg, 3.53 mmol, 5.0 eq) in one portion at 25° C., and the reaction mixture was stirred at 25° C. for 1 hour. The mixture was concentrated, and the pH adjusted to pH=10 by addition of triethylamine. The mixture was purified by flash chromatography on SiO₂ (gradient: 0˜10% methanol (1N NH₃) in dichloromethane) to afford tert-butyl(3S)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (460 mg, crude) as a white solid. LC/MS (ESI) m/z: 764.4 [M+1]⁺. Further purification by chiral SFC [column: DAICEL CHIRALCEL OX (250 mm*30 mm, 10 um); mobile phase: [50% CH₃CN in CH₃OH (0.1% NH₄OH)]]afforded tert-butyl(3S)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (249 mg, 0.280 mmol, 47% yield) as a white solid. LC/MS (ESI) m/z: 764.5 [M+H]⁺.

Step 2: Preparation of (2S,4R)-4-hydroxy-1-[(2R)-3-methyl-2-[3-[4-[[(3R)-3-piperidyl]methyl]piperazin-1-yl]isoxazol-5-yl]butanoyl]-N-[(1S)-1-[4-(4-methylthiazol-5-yl)phenyl]ethyl]pyrrolidine-2-carboxamide

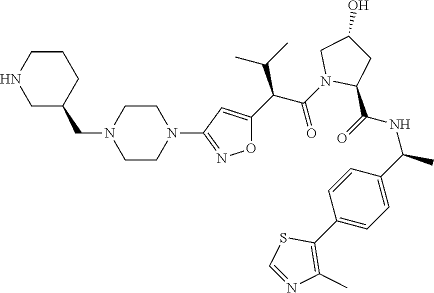 [SMILES: Cc1ncsc1-c1ccc([C@H](C)NC(=O)[C@@H]2C[C@@H](O)CN2C(=O)[C@@H](c2cc(N3CCN(C[C@@H]4CCCNC4)CC3)no2)C(C)C)cc1]

To tert-butyl(3S)-3-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (249 mg, 86% purity, 0.280 mmol, 1.0 eq) in CH₂Cl₂ (3 mL) was added 4 M HCl/dioxane (1.5 mL) in one portion at 25° C., and the reaction mixture was stirred at 25° C. for 30 minutes. The mixture was diluted with petroleum ether (15 mL), and the organic phase was separated. The oily solid was concentrated in vacuum, dissolved in tetrahydrofuran, and treated with triethylamine to adjust the pH (pH=10). The mixture was filtered, and the filtrate was concentrated to afford (2S,4R)-4-hydroxy-1-[(2R)-3-methyl-2-[3-[4-[[(3R)-3-piperidyl]methyl]piperazin-1-yl]isoxazol-5-yl]butanoyl]-N-[(1S)-1-[4-(4-methylthiazol-5-yl)phenyl]ethyl]pyrrolidine-2-carboxamide (217 mg, crude) as a white solid. LC/MS (ESI) m/z: 664.1 [M+H]⁺.

Step 3: Preparation of WS-ARV-JM-098-G-E2, (2S,4R)-1-[(2R)-2-[3-[4-[[(3S)-1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-3-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

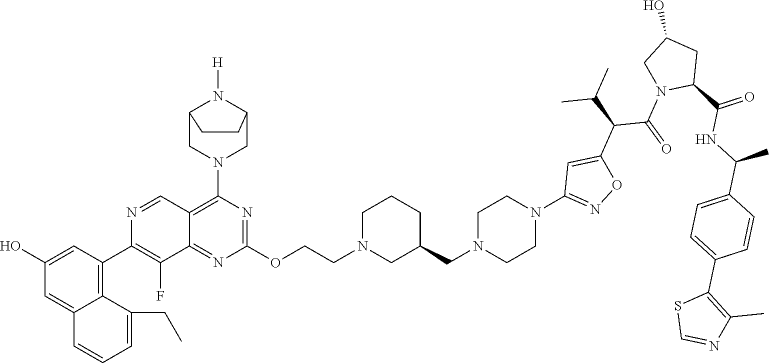 [SMILES: CCc1cccc2cc(O)cc(-c3ncc4c(N5CC6CCC(C5)N6)nc(OCCN5CCC[C@@H](CN6CCN(c7cc([C@H](C(=O)N8C[C@H](O)C[C@H]8C(=O)N[C@@H](C)c8ccc(-c9scnc9C)cc8)C(C)C)on7)CC6)C5)nc4c3F)c12]

The title compound was made in an analogous manner to (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-4-hydroxy-1-[(2R)-3-methyl-2-[3-[4-[[(3R)-3-piperidyl]methyl]piperazin-1-yl]isoxazol-5-yl]butanoyl]-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate. (formic acid salt, white solid). LC/MS (ESI) m/z: 1136.0 [M+H]⁺. ¹H NMR (400 MHz, CD₃OD) δ 9.13-9.04 (m, 1H), 8.93-8.79 (m, 1H), 8.48 (s, 1H), 7.64 (d, J=8.0 Hz, 1H), 7.47-7.34 (m, 4H), 7.34-7.23 (m, 2H), 7.17 (d, J=7.2 Hz, 1H), 7.08-6.99 (m, 1H), 6.12-6.03 (m, 1H), 5.03 (q, J=6.8 Hz, 1H), 4.83-4.67 (m, 5H), 4.57-4.46 (m, 1H), 4.41 (dd, J=1.8, 9.2 Hz, 1H), 4.01 (d, J=6.8 Hz, 2H), 3.92-3.76 (m, 3H), 3.66-3.59 (m, 2H), 3.58-3.48 (m, 2H), 3.43 (s, 2H), 3.17-3.10 (m, 3H), 2.92-2.74 (m, 1H), 2.55 (d, J=6.0 Hz, 3H), 2.48 (s, 2H), 2.46-2.27 (m, 8H), 2.20-2.07 (m, 2H), 2.05-1.80 (m, 8H), 1.55-1.42 (m, 3H), 1.25-1.11 (m, 1H), 1.09-1.01 (m, 3H), 0.93-0.85 (m, 6H).

Exemplary Synthesis of (2S,4R)-1-[(2R)-2-[3-[4-[(3R)-1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-3-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 243)

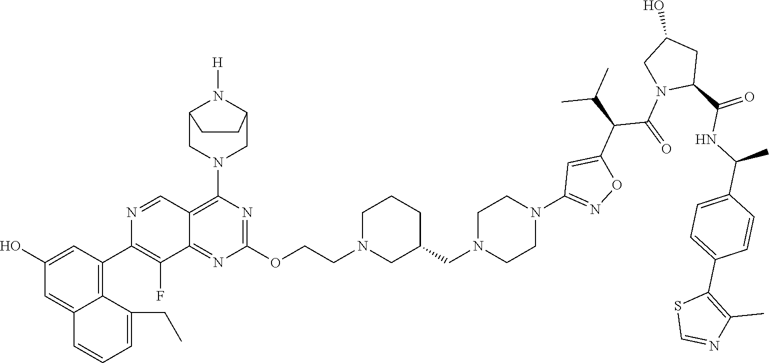 [SMILES: CCc1cccc2cc(O)cc(-c3ncc4c(N5CC6CCC(C5)N6)nc(OCCN5CCC[C@H](CN6CCN(c7cc([C@H](C(=O)N8C[C@H](O)C[C@H]8C(=O)N[C@@H](C)c8ccc(-c9scnc9C)cc8)C(C)C)on7)CC6)C5)nc4c3F)c12]

The title compound was made in an analogous manner to (2S,4R)-1-[(2R)-2-[3-[4-[[(3S)-1-[2-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-3-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from 2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide and tert-butyl(3S)-3-formylpiperidine-1-carboxylate. (white solid). LC/MS (ESI) m/z: 1135.9 [M+H]⁺. ¹H NMR (400 MHz, CD₃OD) δ 9.03 (d, J=2.4 Hz, 1H), 8.87 (s, 1H), 7.62 (d, J=8.0 Hz, 1H), 7.47-7.32 (m, 5H), 7.31-7.26 (m, 1H), 7.16 (d, J=6.8 Hz, 1H), 7.03 (dd, J=8.4, 2.4 Hz, 1H), 6.10-6.01 (m, 1H), 5.08-4.99 (m, 1H), 4.76-4.55 (m, 5H), 4.50 (t, J=8.0 Hz, 1H), 4.45-4.35 (m, 1H), 3.87-3.47 (m, 7H), 3.22-3.11 (m, 4H), 3.07-2.98 (m, 1H), 2.92-2.83 (m, 2H), 2.57-2.13 (m, 15H), 2.01-1.69 (m, 10H), 1.56-1.49 (m, 3H), 1.10-1.00 (m, 3H), 1.00-0.84 (m, 6H).

Exemplary Synthesis of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]azetidin-3-yl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 233)

Step 1: Preparation of tert-butyl 3-(4-piperidylmethyl)azetidine-1-carboxylate

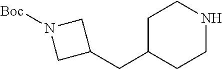 [SMILES: CC(C)(C)OC(=O)N1CC(CC2CCNCC2)C1]

To tert-butyl 3-(4-pyridylmethyl)azetidine-1-carboxylate (2 g, 97% purity, 7.81 mmol, 1.0 eq) in EtOH (40 mL) were added PtO₂ (532 mg, 2.34 mmol, 0.3 eq) and AcOH (894 uL, 15.63 mmol, 2.0 eq), and the reaction mixture was stirred at 70° C. for 20 hours under H₂ (50 psi). The mixture was filtered, and the filtrate was concentrated under reduced pressure. The residue was adjusted to pH ˜12 by adding saturated aqueous Na₂CO₃. The resulting mixture was lyophilized under reduced pressure. CH₂Cl₂ (50 mL) was then added, and the resulting mixture was filtered and concentrated under reduced pressure to afford tert-butyl 3-(4-piperidylmethyl)azetidine-1-carboxylate (1.99 g, crude) as a brown oil. LC/MS (ESI) m/z: 255.1 [M+H]⁺.

Step 2: Preparation of tert-butyl 3-[[1-[5-(1-methoxycarbonyl-2-methyl-propyl)isoxazol-3-yl]-4-piperidyl]methyl]azetidine-1-carboxylate

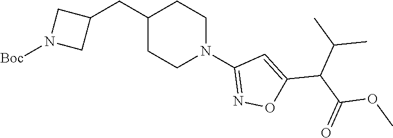 [SMILES: COC(=O)C(c1cc(N2CCC(CC3CN(C(=O)OC(C)(C)C)C3)CC2)no1)C(C)C]

To a mixture of tert-butyl 3-(4-piperidylmethyl)azetidine-1-carboxylate (1.99 g, 7.82 mmol, 1.0 eq), methyl 3-methyl-2-[3-(1,1,2,2,3,3,4,4,4-nonafluorobutylsulfonyloxy)isoxazol-5-yl]butanoate (3.39 g, 7.04 mmol, 0.9 eq), and 4 Å MS (6.5 g) in DMA (40 mL) was added triethylamine (3.3 mL, 23.47 mmol, 3.0 eq), and the reaction mixture was stirred at 130° C. for 3 hours. The reaction mixture was cooled to room temperature, then filtered, washing the cake with EtOAc (200 mL). The mixture was washed with water (50 mL×3), brine (50 mL×3), dried over Na₂SO₄, filtered, and concentrated. The residue was purified by flash chromatography on SiO₂ (gradient: 0-20% ethyl acetate in petroleum ether) to afford tert-butyl 3-[[1-[5-(1-methoxycarbonyl-2-methyl-propyl)isoxazol-3-yl]-4-piperidyl]methyl]azetidine-1-carboxylate (1.96 g, 4.27 mmol, 55% yield) as a yellow oil. LC/MS (ESI) m/z: 436.2 [M+H]⁺. ¹H NMR (400 MHz, CDCl₃) δ 5.92 (s, 1H), 4.02 (t, J=8.4 Hz, 2H), 3.75-3.65 (m, 5H), 3.55 (dd, J=5.6, 8.4 Hz, 2H), 3.48 (d, J=8.8 Hz, 1H), 2.82 (br t, J=12.0 Hz, 2H), 2.68-2.55 (m, 1H), 2.39-2.28 (m, 1H), 2.02-1.75 (m, 3H), 1.67 (br d, J=12.0 Hz, 2H), 1.59 (t, J=7.2 Hz, 2H), 1.44 (s, 9H), 1.00 (d, J=6.8 Hz, 3H), 0.92 (d, J=6.8 Hz, 3H).

Step 3: Preparation of (2S,4R)-1-[(2R)-2-[3-[4-(azetidin-3-ylmethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

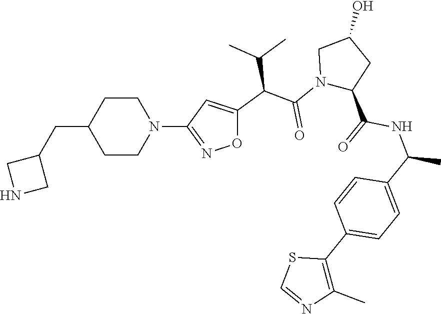 [SMILES: Cc1ncsc1-c1ccc([C@H](C)NC(=O)[C@@H]2C[C@@H](O)CN2C(=O)[C@@H](c2cc(N3CCC(CC4CNC4)CC3)no2)C(C)C)cc1]

The title compound was made in an analogous manner to (2S,4R)-4-hydroxy-1-[(2R)-3-methyl-2-[3-(4-piperidylmethoxy)isoxazol-5-yl]butanoyl]-N-[(1S)-1-[4-(4-methylthiazol-5-yl)phenyl]ethyl]pyrrolidine-2-carboxamide starting from tert-butyl 3-[[1-[5-(1-methoxycarbonyl-2-methyl-propyl)isoxazol-3-yl]-4-piperidyl]methyl]azetidine-1-carboxylate. (yellow solid). LC/MS (ESI) m/z: 635.3 [M+H]⁺.

Step 4: Preparation of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]azetidin-3-yl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

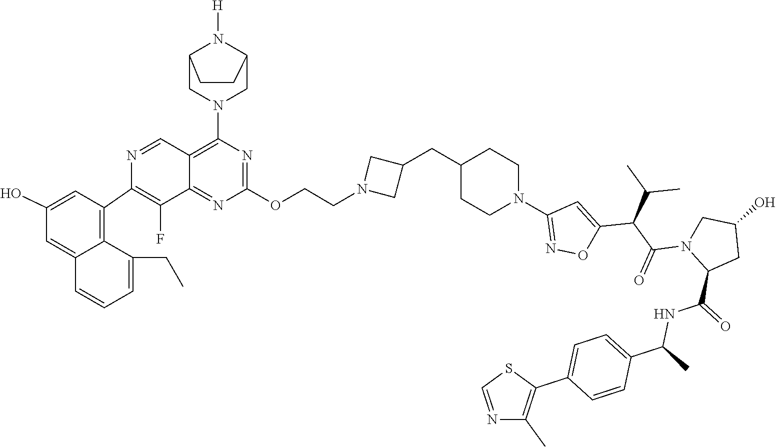 [SMILES: CCc1cccc2cc(O)cc(-c3ncc4c(N5CC6CCC(C5)N6)nc(OCCN5CC(CC6CCN(c7cc([C@H](C(=O)N8C[C@H](O)C[C@H]8C(=O)N[C@@H](C)c8ccc(-c9scnc9C)cc8)C(C)C)on7)CC6)C5)nc4c3F)c12]

The title compound was made in an analogous manner to (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-1-[(2R)-2-[3-[4-(azetidin-3-ylmethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). LC/MS (ESI) m/z: 1106.9 [M+H]⁺. ¹H NMR (400 MHz, DMSO-d₆) δ 10.14-9.71 (m, 1H), 9.07 (s, 1H), 8.98 (s, 1H), 8.48-8.23 (m, 1H), 7.66 (d, J=8.0 Hz, 1H), 7.47-7.40 (m, 2H), 7.40-7.31 (m, 3H), 7.28 (d, J=2.4 Hz, 1H), 7.12 (d, J=7.2 Hz, 1H), 6.97 (d, J=2.4 Hz, 1H), 6.13-5.94 (m, 1H), 5.21-5.00 (m, 1H), 4.90 (quin, J=7.2 Hz, 1H), 4.50-4.17 (m, 6H), 3.75-3.49 (m, 9H), 2.77-2.63 (m, 6H), 2.45 (s, 4H), 2.31-2.06 (m, 4H), 2.05-1.97 (m, 1H), 1.82-1.73 (m, 1H), 1.69-1.54 (m, 6H), 1.48-1.33 (m, 6H), 1.27-1.16 (m, 2H), 1.16-1.05 (m, 2H), 0.98-0.90 (m, 3H), 0.83-0.75 (m, 6H).

Exemplary Synthesis of (2S,4R)-1-[(2S)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]azetidin-3-yl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

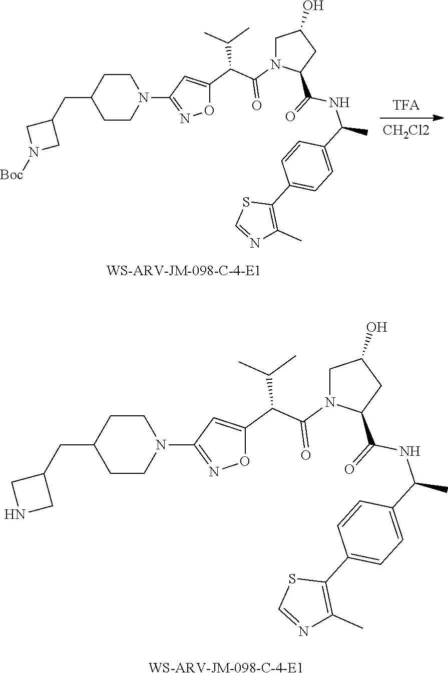 [SMILES: Cc1ncsc1-c1ccc([C@H](C)NC(=O)[C@@H]2C[C@@H](O)CN2C(=O)[C@H](c2cc(N3CCC(CC4CN(C(=O)OC(C)(C)C)C4)CC3)no2)C(C)C)cc1 ; Cc1ncsc1-c1ccc([C@H](C)NC(=O)[C@@H]2C[C@@H](O)CN2C(=O)[C@H](c2cc(N3CCC(CC4CNC4)CC3)no2)C(C)C)cc1]

The title compound was made in an analogous manner to (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]azetidin-3-yl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-1-[(2S)-2-[3-[4-(azetidin-3-ylmethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). LC/MS (ESI) m/z: 1106.9 [M+H]⁺. ¹H NMR (400 MHz, DMSO-d₆) δ 9.91 (br s, 1H), 9.07 (s, 1H), 9.01-8.90 (m, 1H), 8.20 (d, J=7.6 Hz, 1H), 7.66 (d, J=8.0 Hz, 1H), 7.51-7.25 (m, 6H), 7.11 (d, J=7.2 Hz, 1H), 6.97 (d, J=2.4 Hz, 1H), 6.11 (s, 1H), 5.15-4.81 (m, 2H), 4.54-4.35 (m, 3H), 4.32-4.22 (m, 3H), 3.70-3.49 (m, 8H), 3.45-3.37 (m, 3H), 3.26 (br d, J=9.6 Hz, 1H), 2.75-2.58 (m, 6H), 2.48-2.37 (m, 4H), 2.36-1.85 (m, 5H), 1.79 (ddd, J=5.2, 7.2, 12.8 Hz, 1H), 1.68-1.49 (m, 6H), 1.47-1.26 (m, 6H), 1.14-0.99 (m, 2H), 0.95 (d, J=6.4 Hz, 2H), 0.82-0.73 (m, 6H).

Exemplary Synthesis of (2S,4R)-1-[(2R)-2-[3-[6-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-2,6-diazaspiro[3.3]heptan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 235)

Step 1: Preparation of tert-butyl 4-[[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptan-6-yl]methyl]piperidine-1-carboxylate

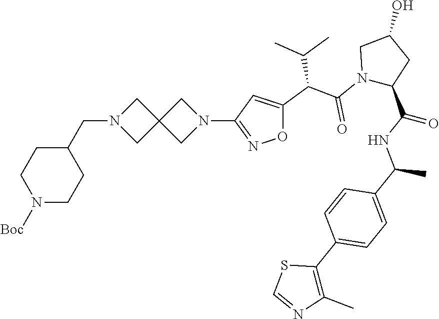 [SMILES: Cc1ncsc1-c1ccc([C@H](C)NC(=O)[C@@H]2C[C@@H](O)CN2C(=O)[C@H](c2cc(N3CC4(CN(CC5CCN(C(=O)OC(C)(C)C)CC5)C4)C3)no2)C(C)C)cc1]

To a solution of (2S,4R)-1-[(2R)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (510 mg, 0.74 mmol, 1.0 eq, TFA) and tert-butyl 4-formylpiperidine-1-carboxylate (236 mg, 1.10 mmol, 1.5 eq) in dichloromethane (5 mL) and isopropanol (5 mL) were added sodium acetate (1.33 g, 16.20 mmol, 22.0 eq) and 2-picoline borane complex (315 mg, 2.94 mmol, 4.0 eq), and the reaction mixture was stirred at 25° C. for 1 hour. The mixture was basified with triethylamine until pH=7 and concentrated under reduced pressure. The residue was purified by flash chromatography on SiO₂ (gradient: 0˜10% methanol in dichloromethane) to afford tert-butyl 4-[[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptan-6-yl]methyl]piperidine-1-carboxylate (650 mg, 0.71 mmol, 96% yield) as alight yellow oil. LC/MS (ESI) m/z: 776.4 [M+H]⁺.

Step 2: Preparation of (2S,4R)-4-hydroxy-1-[(2R)-3-methyl-2-[3-[6-(4-piperidylmethyl)-2,6-diazaspiro[3.3]heptan-2-yl]isoxazol-5-yl]butanoyl]-N-[(1S)-1-[4-(4-methylthiazol-5-yl)phenyl]ethyl]pyrrolidine-2-carboxamide

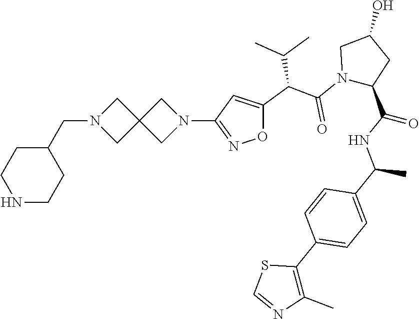 [SMILES: Cc1ncsc1-c1ccc([C@H](C)NC(=O)[C@@H]2C[C@@H](O)CN2C(=O)[C@H](c2cc(N3CC4(CN(CC5CCNCC5)C4)C3)no2)C(C)C)cc1]

To a solution of tert-butyl 4-[[2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2,6-diazaspiro[3.3]heptan-6-yl]methyl]piperidine-1-carboxylate (650 mg, 85% purity, 0.71 mmol, 1.0 eq) in dichloromethane (7.0 mL) was added trifluoroacetic acid (21.24 mmol, 1.6 mL, 30 eq), and the reaction mixture was stirred at 25° C. for 30 minutes. The mixture was concentrated to afford (2S,4R)-4-hydroxy-1-[(2R)-3-methyl-2-[3-[6-(4-piperidylmethyl)-2,6-diazaspiro[3.3]heptan-2-yl]isoxazol-5-yl]butanoyl]-N-[(1S)-1-[4-(4-methylthiazol-5-yl)phenyl]ethyl]pyrrolidine-2-carboxamide (550 mg, crude, TFA) as a light yellow oil. LC/MS (ESI) m/z: 676.3 [M+H]⁺.

Step 3: Preparation of (2S,4R)-1-[(2R)-2-[3-[6-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-2,6-diazaspiro[3.3]heptan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

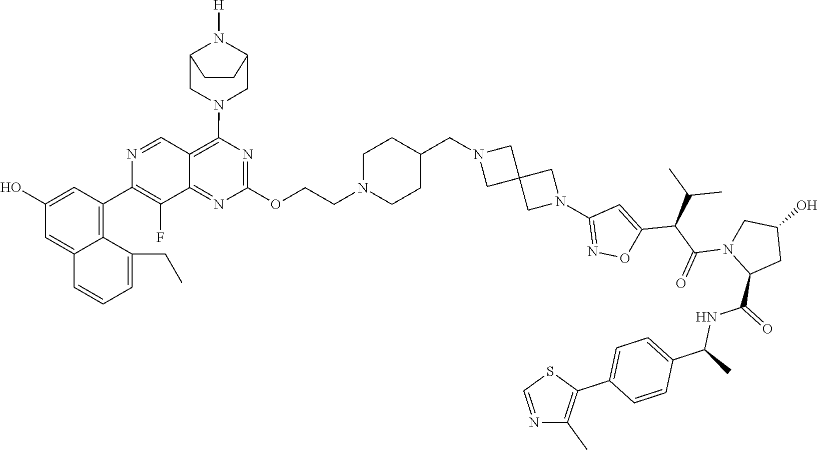 [SMILES: CCc1cccc2cc(O)cc(-c3ncc4c(N5CC6CCC(C5)N6)nc(OCCN5CCC(CN6CC7(C6)CN(c6cc([C@H](C(=O)N8C[C@H](O)C[C@H]8C(=O)N[C@@H](C)c8ccc(-c9scnc9C)cc8)C(C)C)on6)C7)CC5)nc4c3F)c12]

The title compound was made in an analogous manner to (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2R)-3-methyl-2-[3-[6-(4-piperidylmethyl)-2,6-diazaspiro[3.3]heptan-2-yl]isoxazol-5-yl]butanoyl]-N-[(1S)-1-[4-(4-methylthiazol-5-yl)phenyl]ethyl]pyrrolidine-2-carboxamide. (white solid). LC/MS (ESI) m/z: 1148.0 [M+H]⁺. ¹H NMR (400 MHz, CD₃OD) δ 9.04 (s, 1H), 8.88 (s, 1H), 7.64-7.61 (m, 1H), 7.46-7.34 (m, 5H), 7.29-7.28 (m, 1H), 7.17-7.15 (m, 1H), 7.02-7.00 (m, 1H), 5.86-5.81 (m, 1H), 5.04-5.00 (m, 1H), 4.65-4.58 (m, 5H), 4.52-4.43 (m, 2H), 4.01-3.99 (m, 4H), 3.74-3.57 (m, 6H), 3.41 (s, 4H), 3.09-3.06 (m, 2H), 2.87-2.84 (m, 2H), 2.48 (s, 3H), 2.38-2.24 (m, 5H), 2.19-2.13 (m, 3H), 1.98-1.71 (m, 7H), 1.58-1.51 (m, 3H), 1.42-1.37 (m, 1H), 1.29-1.24 (m, 2H), 1.05-1.03 (m, 3H), 0.92-0.86 (m, 6H).

Exemplary Synthesis of (2S,4R)-1-[(2S)-2-[3-[6-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-2,6-diazaspiro[3.3]heptan-2-yl]isoxazol-5-yl]-3-methyl-butanoyl]-4-(Compound 236)

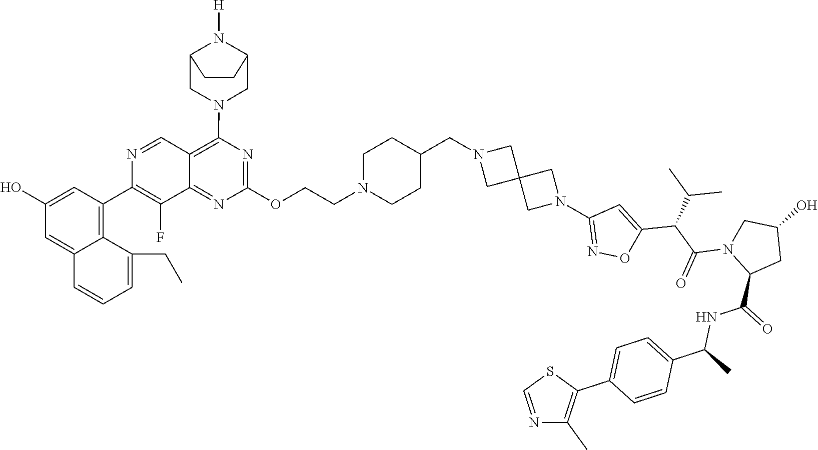 [SMILES: CCc1cccc2cc(O)cc(-c3ncc4c(N5CC6CCC(C5)N6)nc(OCCN5CCC(CN6CC7(C6)CN(c6cc([C@@H](C(=O)N8C[C@H](O)C[C@H]8C(=O)N[C@@H](C)c8ccc(-c9scnc9C)cc8)C(C)C)on6)C7)CC5)nc4c3F)c12]

The title compound was made in an analogous manner to (2S,4R)-1-[(2R)-2-[3-[6-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-2,6-diazaspiro[3.3]heptan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-1-[(2S)-2-[3-(2,6-diazaspiro[3.3]heptan-2-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). LC/MS (ESI) m/z: 1147.5 [M+H]⁺. ¹H NMR (400 MHz, CD₃OD) δ 9.08 (s, 1H), 8.88-8.85 (m, 1H), 7.64-7.62 (m, 1H), 7.47-7.41 (m, 2H), 7.38-7.34 (m, 3H), 7.30-7.29 (m, 1H), 7.17-7.15 (m, 1H), 7.01-7.00 (m, 1H), 5.91-5.85 (m, 1H), 5.01-4.96 (m, 1H), 4.76-4.67 (m, 5H), 4.59-4.53 (m, 1H), 4.42-4.36 (m, 1H), 4.07-4.01 (m, 3H), 3.94-3.93 (m, 2H), 3.86-3.79 (m, 5H), 3.74-3.68 (m, 2H), 3.65-3.60 (m, 1H), 3.51-3.44 (m, 1H), 3.37-3.33 (m, 2H), 3.18-3.15 (m, 2H), 2.70-2.66 (m, 2H), 2.59-2.53 (m, 2H), 2.49-2.46 (m, 3H), 2.39-2.19 (m, 4H), 2.01-1.91 (m, 5H), 1.84-1.81 (m, 2H), 1.66-1.57 (m, 1H), 1.50-1.48 (m, 3H), 1.43-1.31 (m, 2H), 1.05-0.94 (m, 3H), 0.92-0.82 (m, 6H).

Exemplary Synthesis of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 239)

Step 1: Preparation of tert-butyl 4-fluoro-4-(4-piperidylmethyl)piperidine-1-carboxylate

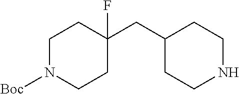 [SMILES: CC(C)(C)OC(=O)N1CCC(F)(CC2CCNCC2)CC1]

To a solution of tert-butyl 4-[(1-benzyloxycarbonyl-4-piperidyl)methyl]-4-fluoro-piperidine-1-carboxylate (2.5 g, 5.75 mmol, 1.0 eq) in EtOH (40 mL) was added Pd/C (0.5 g, 10% purity), and the resulting mixture was degassed under vacuum and purged with H₂ several times, then stirred at 25° C. under H₂ (15 psi) for 2 hours. The mixture was filtered through celite, and the filtrate was concentrated to give tert-butyl 4-fluoro-4-(4-piperidylmethyl)piperidine-1-carboxylate (1.7 g, 98% yield) as a colorless oil.

Step 2: Preparation of (2S,4R)-1-[(2R)-2-[3-[4-[(4-fluoro-4-piperidyl)methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

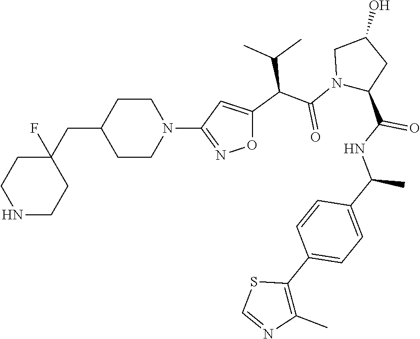 [SMILES: Cc1ncsc1-c1ccc([C@H](C)NC(=O)[C@@H]2C[C@@H](O)CN2C(=O)[C@@H](c2cc(N3CCC(CC4(F)CCNCC4)CC3)no2)C(C)C)cc1]

The title compound was made in an analogous manner to (2S,4R)-1-[(2R)-2-[3-[4-(azetidin-3-ylmethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-fluoro-4-(4-piperidylmethyl)piperidine-1-carboxylate. (light yellow solid). LC/MS (ESI) m/z: 681.5 [M+H]⁺.

Step 3: Preparation of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

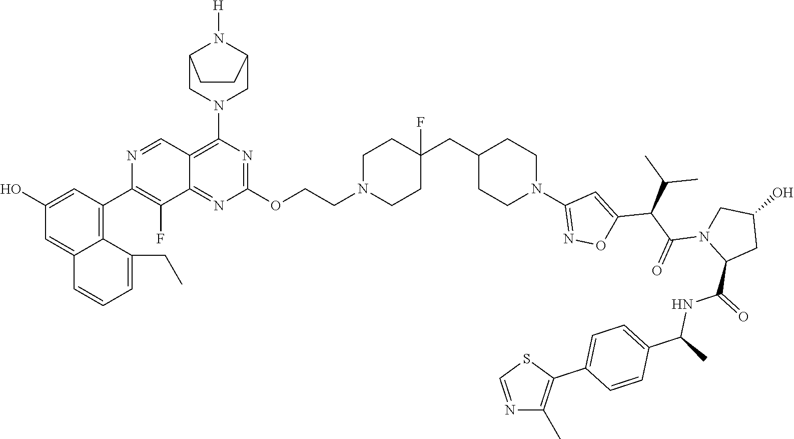 [SMILES: CCc1cccc2cc(O)cc(-c3ncc4c(N5CC6CCC(C5)N6)nc(OCCN5CCC(F)(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-1-[(2R)-2-[3-[4-[(4-fluoro-4-piperidyl)methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate. (formic acid salt, white solid). LC/MS (ESI) m/z: 1153.0 [M+H]⁺. ¹H NMR (400 MHz, CD₃OD) δ 9.08 (s, 1H), 8.93-8.86 (m, 1H), 8.52 (s, 1H), 7.65 (d, J=8.4 Hz, 1H), 7.51-7.28 (m, 6H), 7.18 (d, J=6.8 Hz, 1H), 7.03 (d, J=2.4 Hz, 1H), 6.15-6.01 (m, 1H), 5.10-4.99 (m, 2H), 4.76-4.63 (m, 6H), 4.53 (t, J=8.4 Hz, 1H), 4.46 (s, 1H), 3.96-3.72 (m, 5H), 3.69-3.52 (m, 4H), 3.03 (d, J=5.6 Hz, 4H), 2.85 (t, J=11.6 Hz, 2H), 2.68-2.56 (m, 2H), 2.55-2.47 (m, 3H), 2.42-2.17 (m, 4H), 2.04-1.89 (m, 7H), 1.87-1.66 (m, 5H), 1.60 (d, J=7.2 Hz, 1H), 1.58-1.53 (m, 3H), 1.39-1.26 (m, 2H), 1.07 (d, J=6.4 Hz, 3H), 0.97-0.85 (m, 6H).

Exemplary Synthesis of (2S,4R)-1-[(2S)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 240)

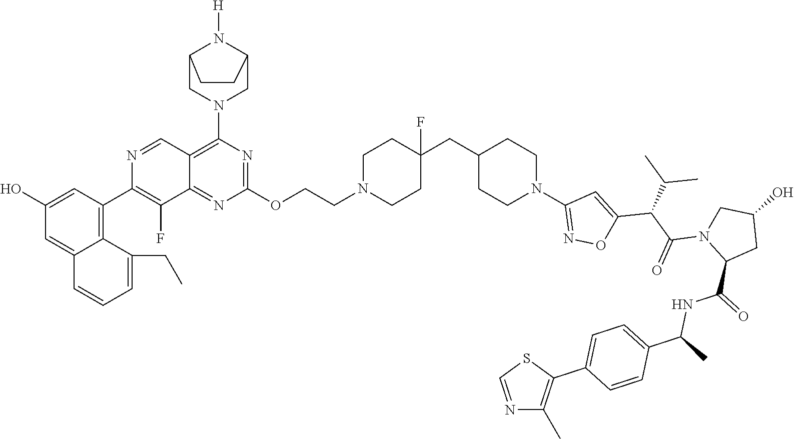 [SMILES: CCc1cccc2cc(O)cc(-c3ncc4c(N5CC6CCC(C5)N6)nc(OCCN5CCC(F)(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-1-[(2S)-2-[3-[4-[(4-fluoro-4-piperidyl)methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate. (formic acid salt, white solid). LC/MS (ESI) m/z: 1153.0 [M+H]⁺. ¹H NMR (400 MHz, CD₃OD) δ 9.06 (s, 1H), 8.90-8.84 (m, 1H), 8.52 (s, 1H), 7.62 (d, J=8.4 Hz, 1H), 7.48-7.28 (m, 6H), 7.16 (d, J=7.2 Hz, 1H), 7.01 (d, J=2.8 Hz, 1H), 6.16-6.02 (m, 1H), 5.04-4.95 (m, 2H), 4.78-4.61 (m, 5H), 4.58 (t, J=8.0 Hz, 1H), 4.43 (s, 1H), 3.92-3.66 (m, 7H), 3.58 (d, J=13.2 Hz, 2H), 3.08-2.93 (m, 4H), 2.77 (t, J=12.4 Hz, 2H), 2.69-2.55 (m, 2H), 2.51-2.43 (m, 3H), 2.39-2.18 (m, 4H), 2.03-2.02 (m, 1H), 2.00-1.86 (m, 7H), 1.82-1.64 (m, 5H), 1.59-1.54 (m, 1H), 1.50-1.46 (m, 3H), 1.24 (d, J=11.6 Hz, 2H), 1.05 (d, J=6.8 Hz, 3H), 0.94-0.85 (m, 6H).

Exemplary Synthesis of (2S,4R)-1-((R)-2-(3-(4-((1-(2-((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)ethyl)-4-methylpiperidin-4-yl)methyl) piperazin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 187)

Step 1: Preparation of tert-butyl 4-formyl-4-methylpiperidine-1-carboxylate

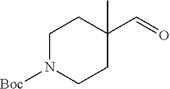 [SMILES: CC1(C=O)CCN(C(=O)OC(C)(C)C)CC1]

To a solution of tert-butyl 4-(hydroxymethyl)-4-methylpiperidine-1-carboxylate (20 g, 87.22 mmol, 1 eq) in dichloromethane (200 mL) at 0° C. was added sodium bicarbonate (14.65 g, 174.43 mmol, 6.8 mL, 2 eq) followed by then Dess-Martin (55.49 g, 130.82 mmol, 40.5 mL, 1.5 eq), and the reaction mixture was stirred at 25° C. for 1 h. The reaction mixture was diluted with a 1:1:1 mixture of water, sodium bicarbonate, and sodium sulfite (180 mL total). The organic phase was washed with sodium bicarbonate (20 mL×3), dried with anhydrous sodium sulfate, filtered, and concentrated. The residue was purified by flash silica gel chromatography (gradient: 0˜8% ethyl acetate in petroleum ether) to afford tert-butyl 4-formyl-4-methylpiperidine-1-carboxylate (15.46 g, 68.02 mmol, 78% yield) as a yellow oil. ¹H NMR (400 MHz, CDCl₃) δ 9.47 (s, 1H), 3.66 (dd, J=4.8, 13.6 Hz, 2H), 3.12 (dd, J=3.6, 13.6 Hz, 2H), 1.91 (dd, J=4.0, 13.6 Hz, 2H), 1.45 (s, 9H), 1.44-1.35 (m, 2H), 1.08 (s, 3H).

Step 2: Preparation of benzyl 4-((1-(tert-butoxycarbonyl)-4-methylpiperidin-4-yl)methyl) piperazine-1-carboxylate

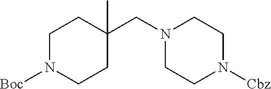 [SMILES: CC1(CN2CCN(C(=O)OCc3ccccc3)CC2)CCN(C(=O)OC(C)(C)C)CC1]

To a solution of benzyl piperazine-1-carboxylate (14.98 g, 68.02 mmol, 13.1 mL, 1 eq) and tert-butyl 4-formyl-4-methylpiperidine-1-carboxylate (15.46 g, 68.02 mmol, 1 eq) in methanol (150 mL) was added acetic acid (6.13 g, 102.02 mmol, 5.8 mL, 1.5 eq), and the resulting mixture was stirred at 70° C. for 12 h. Sodium borohydride acetate (28.83 g, 136.03 mmol, 2 eq) was then added, and the reaction mixture was stirred at 25° C. for 12 h. The mixture was diluted with water (100 mL) and extracted with ethyl acetate (100 mL×2). The combined organic extracts were washed with brine (100 mL×2), dried over anhydrous sodium sulfate, filtered, and concentrated under reduced pressure. The residue was purified by flash silica gel chromatography (gradient: 8˜20% ethyl acetate in petroleum ether) to afford benzyl 4-((1-(tert-butoxycarbonyl)-4-methylpiperidin-4-yl)methyl) piperazine-1-carboxylate (7.7 g, 17.84 mmol, 26% yield) as a colorless oil. LC/MS (ESI) m/z: 432.3 [M+H]⁺. ¹H NMR (400 MHz, CDCl₃) δ 7.40-7.28 (m, 5H), 5.13 (s, 2H), 3.68 (d, J=9.2 Hz, 3H), 3.50-3.42 (m, 4H), 3.21-3.00 (m, 3H), 2.48 (s, 4H), 2.15 (s, 2H), 1.46 (s, 9H), 1.44-1.38 (m, 2H), 0.94 (s, 3H).

Step 3: Preparation of tert-butyl 4-methyl-4-(piperazin-1-ylmethyl)piperidine-1-carboxylate

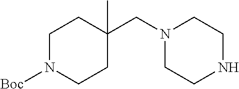 [SMILES: CC1(CN2CCNCC2)CCN(C(=O)OC(C)(C)C)CC1]

To a solution of benzyl 4-((1-(tert-butoxycarbonyl)-4-methylpiperidin-4-yl)methyl) piperazine-1-carboxylate (6.3 g, 14.60 mmol, 1 eq) in tetrahydrofuran (10 mL) and 2,2,2-trifluoroethanol (110 mL) were added palladium on activated carbon (600 mg, 10% purity) and palladium hydroxide on activated carbon (600 mg, 20% purity) under nitrogen, and the suspension was degassed under vacuum and purged with hydrogen several times. The reaction mixture was stirred under hydrogen (50 psi) at 25° C. for 12 hours. The mixture was filtered, washing with ethyl acetate (400 mL), and the filtrate was concentrated to afford tert-butyl 4-methyl-4-(piperazin-1-ylmethyl)piperidine-1-carboxylate (5.1 g, crude) as a colorless oil. ¹H NMR (400 MHz, CDCl₃) δ 3.77-3.58 (m, 2H), 3.14-2.94 (m, 6H), 2.70-2.58 (m, 4H), 2.17 (s, 2H), 1.46 (s, 9H), 1.43-1.33 (m, 2H), 1.26-1.15 (m, 2H), 0.93 (s, 3H).

Step 4: Preparation of (2S,4R)-4-hydroxy-1-((R)-3-methyl-2-(3-(4-((4-methylpiperidin-4-yl)methyl) piperazin-1-yl)isoxazol-5-yl)butanoyl)-N—((S)-1-(4-(4-methylthiazol-5-yl)phenyl)ethyl)pyrrolidine-2-carboxamide

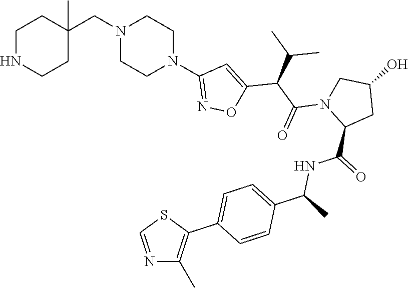 [SMILES: Cc1ncsc1-c1ccc([C@H](C)NC(=O)[C@@H]2C[C@@H](O)CN2C(=O)[C@@H](c2cc(N3CCN(CC4(C)CCNCC4)CC3)no2)C(C)C)cc1]

The title compound was made in an analogous manner to (2S,4R)-1-[(2R)-2-[3-[4-(azetidin-3-ylmethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-methyl-4-(piperazin-1-ylmethyl)piperidine-1-carboxylate. (TFA salt, yellow oil). LC/MS (ESI) m/z: 678.5 [M+H]⁺.

Step 5: Preparation of (2S,4R)-1-((R)-2-(3-(4-((1-(2-((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)ethyl)-4-methylpiperidin-4-yl)methyl) piperazin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide

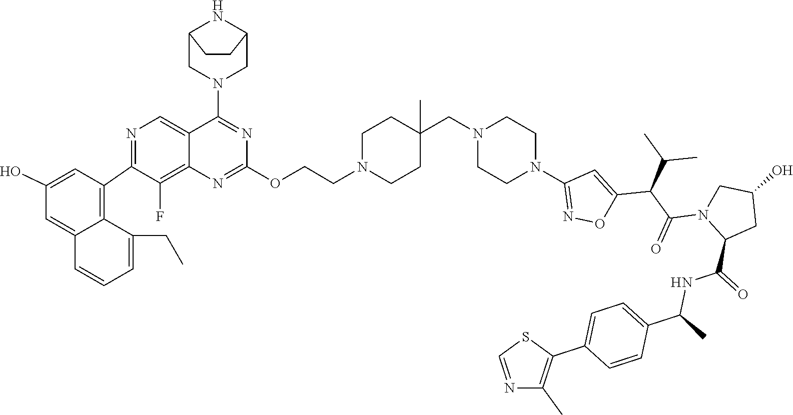 [SMILES: CCc1cccc2cc(O)cc(-c3ncc4c(N5CC6CCC(C5)N6)nc(OCCN5CCC(C)(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-4-hydroxy-1-((R)-3-methyl-2-(3-(4-((4-methyl piperidin-4-yl)methyl) piperazin-1-yl)isoxazol-5-yl)butanoyl)-N—((S)-1-(4-(4-methylthiazol-5-yl)phenyl)ethyl)pyrrolidine-2-carboxamide and tert-butyl(1R,5S)-3-(7-(8-ethyl-3-((tetrahydro-2H-pyran-2-yl)oxy)naphthalen-1-yl)-8-fluoro-2-(2-oxoethoxy)pyrido[4,3-d]pyrimidin-4-yl)-3,8-diazabicyclo[3.2.1]octane-8-carboxylate. (white solid). LC/MS (ESI) m/z: 1149.8 [M+H]⁺. ¹H NMR (400 MHz, CD₃OD) δ 9.04 (s, 1H), 8.87 (s, 1H), 7.63 (d, J=8.3 Hz, 1H), 7.48-7.39 (m, 4H), 7.39-7.33 (m, 1H), 7.29 (d, J=2.4 Hz, 1H), 7.16 (d, J=7.2 Hz, 1H), 7.01 (d, J=2.8 Hz, 1H), 6.10-5.99 (m, 1H), 5.03 (q, J=7.2 Hz, 1H), 4.69-4.63 (m, 4H), 4.51 (t, J=8.2 Hz, 1H), 4.43 (d, J=1.2 Hz, 1H), 3.88-3.81 (m, 1H), 3.78-3.70 (m, 2H), 3.69-3.63 (m, 3H), 3.62-3.57 (m, 1H), 3.20 (s, 4H), 2.90 (t, J=5.2 Hz, 2H), 2.77 (d, J=2.4 Hz, 2H), 2.66 (s, 1H), 2.61 (s, 4H), 2.58-2.50 (m, 2H), 2.49-2.46 (m, 3H), 2.41-2.25 (m, 3H), 2.24-2.19 (m, 2H), 2.19-2.14 (m, 1H), 2.07-1.91 (m, 1H), 1.91-1.82 (m, 2H), 1.82-1.75 (m, 2H), 1.73-1.62 (m, 2H), 1.60-1.49 (m, 3H), 1.42-1.28 (m, 3H), 1.05 (d, J=6.4 Hz, 3H), 0.96 (s, 3H), 0.94-0.86 (m, 6H).

Exemplary Synthesis of (2S,4R)-1-((S)-2-(3-(4-((1-(2-((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)ethyl)-4-methylpiperidin-4-yl)methyl) piperazin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Compound 188)

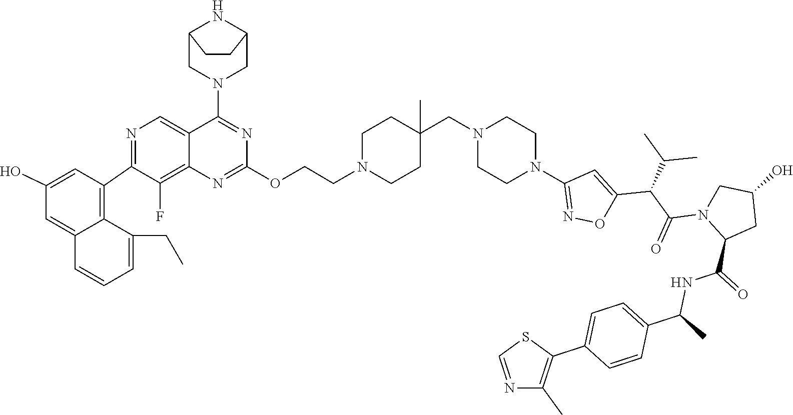 [SMILES: CCc1cccc2cc(O)cc(-c3ncc4c(N5CC6CCC(C5)N6)nc(OCCN5CCC(C)(CN6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-((R)-2-(3-(4-((1-(2-((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)ethyl)-4-methylpiperidin-4-yl)methyl) piperazin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide starting from (2S,4R)-4-hydroxy-1-((S)-3-methyl-2-(3-(4-((4-methyl piperidin-4-yl)methyl) piperazin-1-yl)isoxazol-5-yl)butanoyl)-N—((S)-1-(4-(4-methylthiazol-5-yl)phenyl)ethyl)pyrrolidine-2-carboxamide and tert-butyl(1R,5S)-3-(7-(8-ethyl-3-((tetrahydro-2H-pyran-2-yl)oxy)naphthalen-1-yl)-8-fluoro-2-(2-oxoethoxy)pyrido[4,3-d]pyrimidin-4-yl)-3,8-diazabicyclo[3.2.1]octane-8-carboxylate. (white solid). LC/MS (ESI) m/z: 1150.9 [M+H]⁺. ¹H NMR (400 MHz, CD₃OD) δ 9.04 (s, 1H), 8.93-8.81 (m, 1H), 7.62 (d, J=8.4 Hz, 1H), 7.51-7.43 (m, 1H), 7.43-7.39 (m, 2H), 7.39-7.32 (m, 3H), 7.28 (d, J=2.4 Hz, 1H), 7.16 (d, J=7.2 Hz, 1H), 7.01 (d, J=2.4 Hz, 1H), 6.15-6.00 (m, 1H), 5.03-4.97 (m, 2H), 4.69-4.63 (m, 5H), 4.43 (s, 1H), 3.77-3.70 (m, 3H), 3.70-3.62 (m, 4H), 3.24-3.15 (m, 4H), 2.89 (t, J=5.6 Hz, 2H), 2.76 (d, J=6.4 Hz, 2H), 2.64-2.59 (m, 1H), 2.59-2.50 (m, 5H), 2.49 (s, 1H), 2.47 (s, 2H), 2.43-2.21 (m, 5H), 2.19 (s, 2H), 1.97 (dd, J=4.8, 13.2 Hz, 1H), 1.91-1.82 (m, 2H), 1.82-1.75 (m, 2H), 1.64 (t, J=10.0 Hz, 2H), 1.60-1.46 (m, 3H), 1.39-1.28 (m, 2H), 1.05 (d, J=6.8 Hz, 3H), 0.97-0.94 (m, 3H), 0.93-0.83 (m, 6H).

Exemplary Synthesis of (2S,4R)-1-[(2R)-2-[3-[[3-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 185)

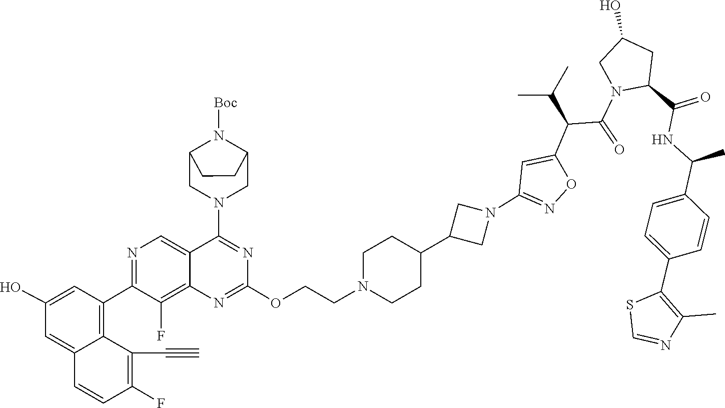 [SMILES: C#Cc1c(F)ccc2cc(O)cc(-c3ncc4c(N5CC6CCC(C5)N6C(=O)OC(C)(C)C)nc(OCCN5CCC(C6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was made in an analogous manner to (2S,4R)-1-[(2R)-2-[3-[7-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-4-hydroxy-1-[(2R)-3-methyl-2-[3-[[3-(4-piperidyl) azetidin-1-yl]isoxazol-5-yl]butanoyl]-N-[(1S)-1-[4-(4-methylthiazol-5-yl)phenyl]ethyl]pyrrolidine-2-carboxamide and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate. (yellow solid). LC/MS (ESI) m/z: 1107.8 [M+H]⁺. ¹H NMR (400 MHz, CD₃OD) δ 9.00 (s, 1H), 8.87 (s, 1H), 7.85 (dd, J=5.6, 9.2 Hz, 1H), 7.51-7.38 (m, 4H), 7.37-7.28 (m, 2H), 7.21 (d, J=2.4 Hz, 1H), 5.88-5.71 (m, 1H), 5.06-4.96 (m, 1H), 4.68-4.59 (m, 3H), 4.58 (s, 2H), 4.46-4.36 (m, 1H), 3.98 (t, J=7.6 Hz, 2H), 3.83 (dd, J=4.4, 10.8 Hz, 1H), 3.77-3.68 (m, 2H), 3.68-3.62 (m, 5H), 3.62-3.44 (m, 2H), 3.37 (s, 1H), 3.09 (s, 2H), 2.85 (t, J=5.6 Hz, 2H), 2.60-2.50 (m, 1H), 2.47 (s, 3H), 2.39-2.26 (m, 1H), 2.17 (t, J=11.2 Hz, 3H), 1.95 (dd, J=4.8, 8.8 Hz, 1H), 1.89-1.75 (m, 4H), 1.70 (d, J=12.0 Hz, 2H), 1.60-1.46 (m, 4H), 1.34-1.15 (m, 3H), 1.04 (d, J=6.8 Hz, 3H), 0.93-0.82 (m, 3H).

Exemplary Synthesis of (2S,4R)-1-[(2R)-2-[3-[[3-[2-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]ethyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 184)

Step 1: Preparation of benzyl 3-formylazetidine-1-carboxylate

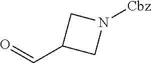 [SMILES: O=CC1CN(C(=O)OCc2ccccc2)C1]

To a mixture of benzyl 3-(hydroxymethyl)azetidine-1-carboxylate (10 g, 45.20 mmol, 1 eq) and sodium bicarbonate (7.5 g, 89.28 mmol, 1.98 eq) in dichloromethane (300 mL) at 0° C. was added Dess-Martin (24.92 g, 58.76 mmol, 1.3 eq) under nitrogen, and the reaction mixture was stirred at 25° C. for 1 h. The mixture was diluted with dichloromethane (100 mL), saturated sodium thiosulfate (50 mL), saturated sodium bicarbonate (50 mL), and water (50 mL), and the resulting mixture was stirred at 25° C. for 15 minutes. The aqueous phase was extracted with dichloromethane (100 mL×2), and the combined organic extract was washed with brine (200 mL×2), dried over anhydrous sodium sulfate, filtered, and concentrated to afford benzyl 3-formylazetidine-1-carboxylate (9.8 g, crude) as a yellow oil.

Step 2: Preparation of tert-butyl 4-[(E)-2-(1-benzyloxycarbonylazetidin-3-yl)vinyl]piperidine-1-carboxylate

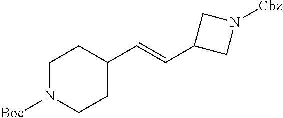 [SMILES: CC(C)(C)OC(=O)N1CCC(/C=C/C2CN(C(=O)OCc3ccccc3)C2)CC1]

To a solution of tert-butyl 4-[[iodo(triphenyl)-25-phosphanyl]methyl]piperidine-1-carboxylate (10.72 g, 18.25 mmol, 1 eq) in tetrahydrofuran (100 mL) at 0° C. was added lithium bis(trimethylsilyl)amide (1 M, 16 mL), and the mixture was stirred at 20° C. for 1 hour. Benzyl 3-formylazetidine-1-carboxylate (4.0 g, 18.25 mmol, 1 eq) in tetrahydrofuran (15 mL) was then added to the mixture at 0° C., and the reaction mixture was stirred at 0° C. for 1 h, then at 20° C. 10 h. Saturated aqueous solution of ammonium chloride (500 mL) was added into the mixture and stirred for 30 minutes. The aqueous phase was extracted with ethyl acetate (400 mL×3), and the combined organic extract was washed with brine (300 mL×3), dried over anhydrous sodium sulfate, filtered, and concentrated. The residue was purified by silica gel column chromatography (petroleum ether/ethyl acetate=50/1 to 10:1) to afford tert-butyl 4-[(E)-2-(1-benzyloxycarbonylazetidin-3-yl) vinyl]piperidine-1-carboxylate (3.1 g, 7.74 mmol, 42% yield) as a yellow oil. ¹H NMR (400 MHz, CDCl₃) δ 7.40-7.29 (m, 5H), 5.59 (t, J=10.1 Hz, 1H), 5.28 (s, 1H), 5.10 (s, 2H), 4.28-4.16 (m, 2H), 4.11-4.02 (m, 2H), 3.77 (dd, J=5.9, 8.4 Hz, 2H), 3.59-3.15 (m, 1H), 2.82-2.65 (m, 2H), 2.28 (br d, J=9.4 Hz, 1H), 1.54-1.41 (m, 11H), 1.33-1.21 (m, 3H).

Step 3: Preparation of WX-ARV-JM-044-H-3, tert-butyl 4-[2-(azetidin-3-yl)ethyl]piperidine-1-carboxylate

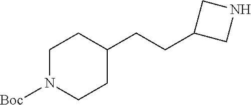 [SMILES: CC(C)(C)OC(=O)N1CCC(CCC2CNC2)CC1]

To a solution of tert-butyl 4-[(E)-2-(1-benzyloxycarbonylazetidin-3-yl) vinyl]piperidine-1-carboxylate (6 g, 14.98 mmol, 1 eq) in trifluoroethanol (60 mL) and tetrahydrofuran (60 mL) was added palladium on activated carbon catalyst (2 g, 14.98 mmol, 10% purity) under nitrogen, and the suspension was degassed under vacuum and purged with hydrogen several times. The reaction mixture was stirred under hydrogen (20 Psi) at 25° C. for 12 hours. The mixture was filtered and concentrated under reduced pressure at 45° C. to afford tert-butyl 4-[2-(azetidin-3-yl)ethyl]piperidine-1-carboxylate (4 g, crude) as a colorless oil.

Step 4: Preparation of (2S,4R)-4-hydroxy-1-[(2R)-3-methyl-2-[3-[[3-[2-(4-piperidyl)ethyl]azetidin-1-yl]isoxazol-5-yl]butanoyl]-N-[(1S)-1-[4-(4-methylthiazol-5-yl)phenyl]ethyl]pyrrolidine-2-carboxamide

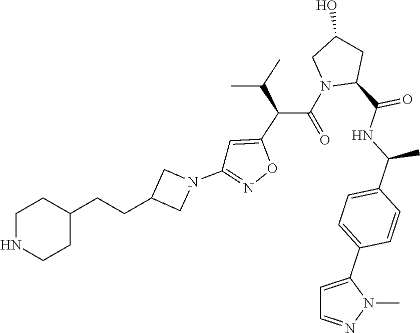 [SMILES: CC(C)[C@@H](C(=O)N1C[C@H](O)C[C@H]1C(=O)N[C@@H](C)c1ccc(-c2ccnn2C)cc1)c1cc(N2CC(CCC3CCNCC3)C2)no1]

The title compound was made in an analogous manner to (2S,4R)-1-[(2R)-2-[3-[4-(azetidin-3-ylmethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from tert-butyl 4-[2-(azetidin-3-yl)ethyl]piperidine-1-carboxylate. (TFA salt, light yellow gum). LC/MS (ESI) m/z: 649.4 [M+H]⁺.

Step 5: Preparation of (2S,4R)-1-[(2R)-2-[3-[[3-[2-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]ethyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

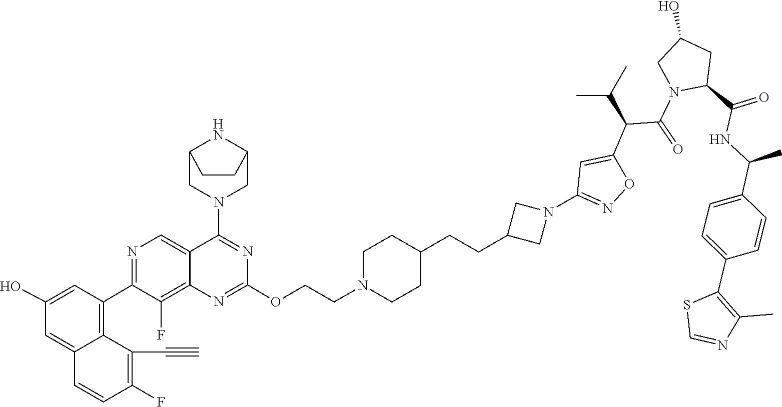 [SMILES: C#Cc1c(F)ccc2cc(O)cc(-c3ncc4c(N5CC6CCC(C5)N6)nc(OCCN5CCC(CCC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was made in an analogous manner to (2S,4R)-1-[(2R)-2-[3-[7-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-4-hydroxy-1-[(2R)-3-methyl-2-[3-[[3-[2-(4-piperidyl)ethyl]azetidin-1-yl]isoxazol-5-yl]butanoyl]-N-[(1S)-1-[4-(4-methylthiazol-5-yl)phenyl]ethyl]pyrrolidine-2-carboxamide and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate. (yellow solid). LC/MS (ESI) m/z: 1134.8 [M+H]⁺. ¹H NMR (400 MHz, CD₃OD) δ 9.01 (s, 1H), 8.87 (s, 1H), 7.85 (dd, J=5.6, 9.2 Hz, 1H), 7.49-7.39 (m, 4H), 7.36-7.27 (m, 2H), 7.21 (d, J=2.4 Hz, 1H), 5.90-5.73 (m, 1H), 5.10-4.95 (m, 1H), 4.71-4.56 (m, 4H), 4.51 (t, J=8.0 Hz, 1H), 4.46-4.37 (m, 1H), 4.02 (br t, J=7.6 Hz, 2H), 3.83 (dd, J=4.0, 10.4 Hz, 1H), 3.76-3.62 (m, 5H), 3.61 (s, 1H), 3.58-3.44 (m, 3H), 3.37 (s, 1H), 3.14-3.00 (m, 2H), 2.86 (br t, J=5.6 Hz, 2H), 2.77-2.67 (m, 1H), 2.48 (s, 3H), 2.41-2.27 (m, 1H), 2.24-2.11 (m, 3H), 2.05-1.91 (m, 1H), 1.89-1.70 (m, 6H), 1.69-1.62 (m, 2H), 1.60-1.48 (m, 3H), 1.39-1.14 (m, 6H), 1.04 (d, J=6.4 Hz, 3H), 0.96-0.83 (m, 3H).

Exemplary Synthesis of 5-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide (Compound 293)

Step 1: Preparation of methyl 5-[4-[(1-tert-butoxycarbonyl-4-piperidyl)methyl]piperazin-1-yl]pyrazine-2-carboxylate

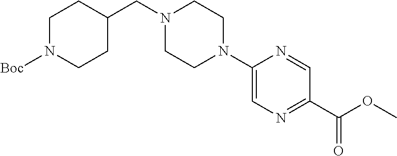 [SMILES: COC(=O)c1cnc(N2CCN(CC3CCN(C(=O)OC(C)(C)C)CC3)CC2)cn1]

To a solution of methyl 5-chloropyrazine-2-carboxylate (1 g, 5.79 mmol, 1.0 eq) in DMF (10 mL) were added tert-butyl 4-(piperazin-1-ylmethyl)piperidine-1-carboxylate (1.8 g, 6.37 mmol, 1.1 eq) and triethylamine (1.6 mL, 11.59 mmol, 2.0 eq), and the reaction mixture was stirred at 80° C. for 3 hours. The reaction mixture was diluted with water (50 mL) and extracted with CH₂Cl₂ (50 mL×3). The combined extracts were washed with brine (15 mL×3), dried over anhydrous Na₂SO₄, filtered, and concentrated under reduced pressure. The residue was purified by flash chromatography on SiO₂ (gradient: 0˜50% THF in petroleum ether) to afford methyl 5-[4-[(1-tert-butoxycarbonyl-4-piperidyl)methyl]piperazin-1-yl]pyrazine-2-carboxylate (2.2 g, 5.24 mmol, 91% yield) as a yellow solid. LC/MS (ESI) m/z: 420.0 [M+H]⁺.

Step 2: Preparation of 5-[4-[(1-tert-butoxycarbonyl-4-piperidyl)methyl]piperazin-1-yl]pyrazine-2-carboxylic acid

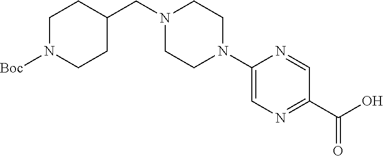 [SMILES: CC(C)(C)OC(=O)N1CCC(CN2CCN(c3cnc(C(=O)O)cn3)CC2)CC1]

To a solution of methyl 5-[4-[(1-tert-butoxycarbonyl-4-piperidyl)methyl]piperazin-1-yl]pyrazine-2-carboxylate (2.2 g, 5.24 mmol, 1.0 eq) in THF (20 mL) and H₂O (5 mL) was added LiOH·H₂O (1.10 g, 26.22 mmol, 5.0 eq), and the reaction mixture was stirred at 25° C. for 10 hours. The mixture was concentrated, diluted with water (20 mL), and acidified by addition of 2N HCl until pH ˜4. The resulting aqueous mixture was lyophilized to afford 5-[4-[(1-tert-butoxycarbonyl-4-piperidyl)methyl]piperazin-1-yl]pyrazine-2-carboxylic acid (2 g, crude) as a yellow solid. LC/MS (ESI) m/z: 406.0 [M+H]⁺.

Step 3: Preparation of tert-butyl 4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]piperidine-1-carboxylate

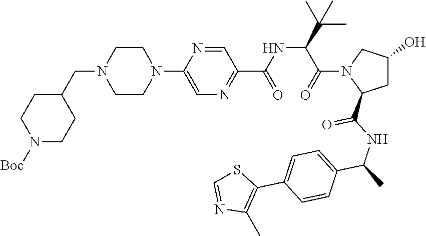 [SMILES: Cc1ncsc1-c1ccc([C@H](C)NC(=O)[C@@H]2C[C@@H](O)CN2C(=O)[C@@H](NC(=O)c2cnc(N3CCN(CC4CCN(C(=O)OC(C)(C)C)CC4)CC3)cn2)C(C)(C)C)cc1]

To a solution of 5-[4-[(1-tert-butoxycarbonyl-4-piperidyl)methyl]piperazin-1-yl]pyrazine-2-carboxylic acid (500 mg, 1.23 mmol, 1.0 eq) in CH₂Cl₂ (15 mL) were added DIEA (2.2 mL, 12.33 mmol, 10.0 eq), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (861 mg HCl salt, 70% purity, 1.36 mmol, 1.1 eq), and HATU (516 mg, 1.36 mmol, 1.1 eq), and the reaction mixture was stirred at 25° C. for 10 hours. The reaction mixture was diluted with water (50 mL) and extracted with CH₂Cl₂ (70 mL×3). The combined organic extracts were washed with brine (100 mL×2), dried over anhydrous Na₂SO₄, filtered, and concentrated under reduced pressure. The residue was purified by flash chromatography on SiO₂ (gradient: 0˜10% CH₃OH in CH₂Cl₂) to afford tert-butyl 4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]piperidine-1-carboxylate (374 mg, 0.449 mmol, 36% yield) as a yellow solid. LC/MS (ESI) m/z: 832.2 [M+H]⁺.

Step 4: Preparation of N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]-5-[4-(4-piperidylmethyl) piperazin-1-yl]pyrazine-2-carboxamide

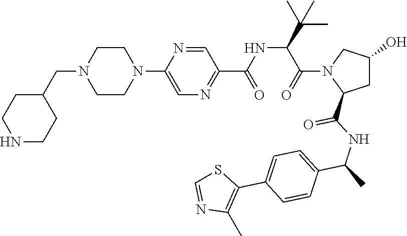 [SMILES: Cc1ncsc1-c1ccc([C@H](C)NC(=O)[C@@H]2C[C@@H](O)CN2C(=O)[C@@H](NC(=O)c2cnc(N3CCN(CC4CCNCC4)CC3)cn2)C(C)(C)C)cc1]

A solution of tert-butyl 4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]piperidine-1-carboxylate (374 mg, 0.449 mmol, 1.0 eq) in CH₂Cl₂ (3 mL) and 4 M HCl/dioxane (3 mL) was stirred at 25° C. for 1 hour. The mixture was concentrated, diluted with H₂O (10 mL), and basified with saturated aqueous NaHCO₃ solution until pH ˜8. The suspension was extracted with CH₂Cl₂/CH₃OH (40 mL×7, V/V=10/1), and the combined organic extracts were dried over anhydrous Na₂SO₄, filtered, and concentrated under reduced pressure to afford N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]-5-[4-(4-piperidylmethyl) piperazin-1-yl]pyrazine-2-carboxamide (331 mg, crude) as a yellow solid. LC/MS (ESI) m/z: 732.2 [M+H]⁺.

Step 5: Preparation of tert-butyl 3-[7-(8-ethyl-3-hydroxy-1-naphthyl)-8-fluoro-2-[2-[[4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]-1-piperidyl]ethoxy]pyrido[4,3-d]pyrimidin-4-yl]-3,8-diazabicyclo[3.2.1]octane-8-carboxylate

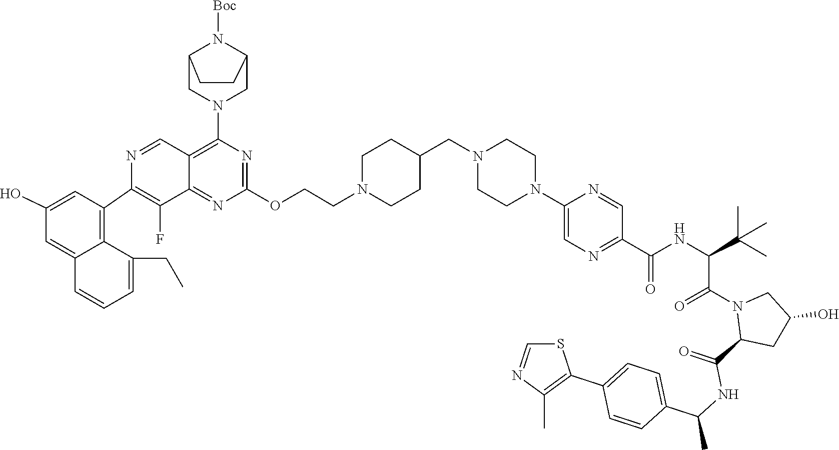 [SMILES: CCc1cccc2cc(O)cc(-c3ncc4c(N5CC6CCC(C5)N6C(=O)OC(C)(C)C)nc(OCCN5CCC(CN6CCN(c7cnc(C(=O)N[C@H](C(=O)N8C[C@H](O)C[C@H]8C(=O)N[C@@H](C)c8ccc(-c9scnc9C)cc8)C(C)(C)C)cn7)CC6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (265 mg, 0.451 mmol, 1.0 eq) and N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]-5-[4-(4-piperidylmethyl) piperazin-1-yl]pyrazine-2-carboxamide (330 mg, 0.451 mmol, 1.0 eq) in CH₂Cl₂ (5 mL) and isopropanol (5 mL) were added AcOH (0.129 mL, 2.25 mmol, 5.0 eq) and 2-methylpyridine borane (241 mg, 2.25 mmol, 5.0 eq), and the reaction mixture was stirred at 25° C. for 1 hour. The mixture was basified with triethylamine until pH ˜8 and concentrated under reduced pressure. The residue was purified by flash chromatography on SiO₂ (gradient: 0˜7% CH₃OH in CH₂Cl₂) to afford tert-butyl 3-[7-(8-ethyl-3-hydroxy-1-naphthyl)-8-fluoro-2-[2-[[4-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]-1-piperidyl]ethoxy]pyrido[4,3-d]pyrimidin-4-yl]-3,8-diazabicyclo[3.2.1]octane-8-carboxylate (390 mg, 0.299 mmol, 66% yield) as a yellow solid. LC/MS (ESI) m/z: 1303.5 [M+H]⁺.

Step 6: Preparation of 5-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide

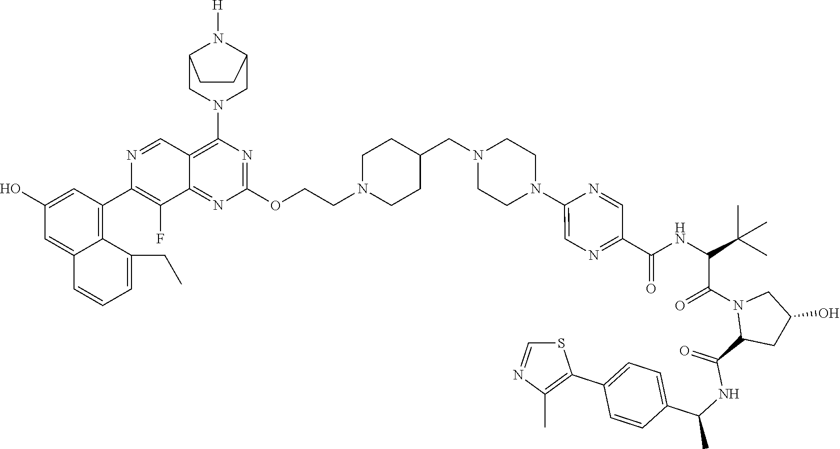

A solution of tert-butyl 3-[7-(8-ethyl-3-hydroxy-1-naphthyl)-8-fluoro-2-[2-[[4-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]-1-piperidyl]ethoxy]pyrido[4,3-d]pyrimidin-4-yl]-3,8-diazabicyclo[3.2.1]octane-8-carboxylate (390 mg, 0.299 mmol, 1.0 eq) in CH₂Cl₂ (3 mL) and 4 M HCl/dioxane (3 mL) was stirred at 25° C. for 1 hour. The mixture was diluted with petroleum ether (10 mL) and the precipitate was filtered. The solid was dissolved in THF (5 ml), basified with triethylamine (0.3 mL) and then filtered. The filtrate was concentrated under reduced pressure, and the resulting residue was purified by prep-HPLC {column: Phenomenex C18 75*30 mm*3 um; mobile phase: [5-45% CH₃CN in water (formic acid)]}. Pure fraction was lyophilized to afford 5-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide (201.2 mg, 0.161 mmol, 54% yield, formic acid salt) as a white solid. LC/MS (ESI) m/z: 1204.0 [M+H]⁺. ¹H NMR (400 MHz, CD₃OD) δ 9.11 (s, 1H), 8.90-8.85 (m, 1H), 8.70-8.65 (m, 1H), 8.45 (s, 2H), 8.24-8.18 (m, 1H), 7.64 (d, J=8.4 Hz, 1H), 7.47-7.42 (m, 3H), 7.42-7.34 (m, 2H), 7.30 (d, J=2.4 Hz, 1H), 7.18 (d, J=7.2 Hz, 1H), 7.01 (d, J=2.4 Hz, 1H), 5.02 (d, J=7.2 Hz, 1H), 4.87-4.79 (m, 6H), 4.77-4.70 (m, 2H), 4.58 (t, J=8.4 Hz, 1H), 4.46 (s, 1H), 4.06-3.98 (m, 2H), 3.97-3.80 (m, 4H), 3.76 (s, 4H), 3.62 (d, J=11.2 Hz, 2H), 3.47 (s, 2H), 2.93 (t, J=11.2 Hz, 2H), 2.56 (d, J=4.0 Hz, 4H), 2.50-2.46 (m, 3H), 2.36-2.18 (m, 5H), 2.08-1.96 (m, 7H), 1.52 (d, J=7.2 Hz, 3H), 1.11-1.06 (m, 9H), 0.90 (t, J=7.2 Hz, 3H).

Exemplary Synthesis of 5-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide (Compound 271)

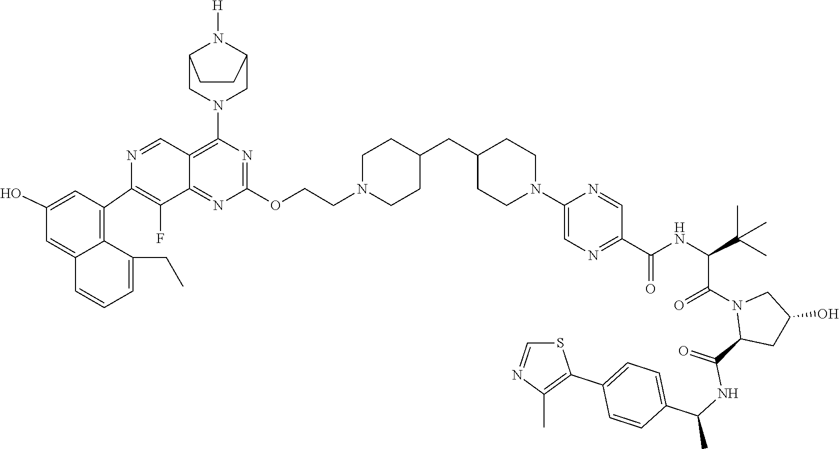 [SMILES: CCc1cccc2cc(O)cc(-c3ncc4c(N5CC6CCC(C5)N6)nc(OCCN5CCC(CC6CCN(c7cnc(C(=O)N[C@H](C(=O)N8C[C@H](O)C[C@H]8C(=O)N[C@@H](C)c8ccc(-c9scnc9C)cc8)C(C)(C)C)cn7)CC6)CC5)nc4c3F)c12]

The title compound was made in an analogous manner to 5-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide starting from tert-butyl 4-(4-piperidylmethyl)piperidine-1-carboxylate. (HCl salt, yellow solid). LC/MS (ESI) m/z: 1203.1. [M+H]⁺. ¹H NMR (400 MHz, CD₃OD) δ 9.07 (s, 1H), 8.89 (s, 1H), 8.66 (d, J=1.2 Hz, 1H), 8.22 (s, 1H), 7.65 (d, J=8.0 Hz, 1H), 7.48-7.36 (m, 5H), 7.30 (d, J=2.4 Hz, 1H), 7.18 (d, J=8.0 Hz, 1H), 7.03 (d, J=2.8 Hz, 1H), 5.03 (q, J=1.2 Hz, 1H), 4.69-4.55 (m, 10H), 3.95 (d, J=1.2 Hz, 1H), 3.84-3.70 (m, 5H), 3.19-3.15 (m, 2H), 3.05-2.96 (m, 4H), 2.50 (s, 1H), 2.40-2.20 (m, 4H), 1.93-1.75 (m, 9H), 1.65-1.50 (m, 4H), 1.35-1.18 (m, 7H), 1.11 (s, 9H), 0.92 (t, J=7.6 Hz, 1H).

Exemplary Synthesis of 5-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide (Compound 279)

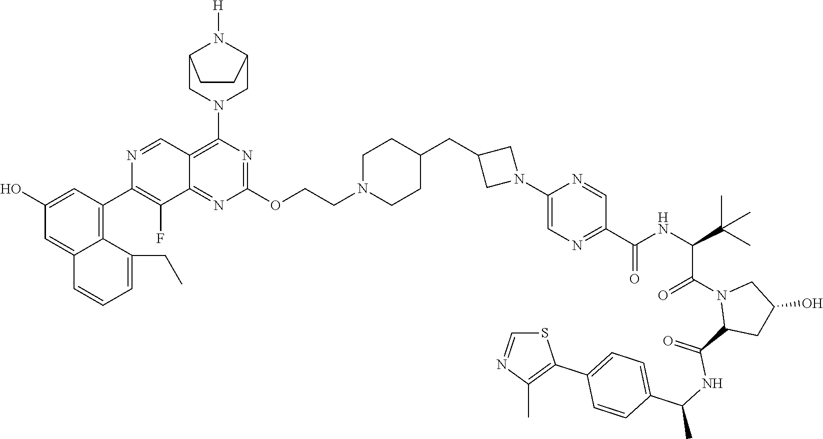

The title compound was made in an analogous manner to 5-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide starting from tert-butyl 4-(azetidin-3-ylmethyl)piperidine-1-carboxylate. (formic acid salt, yellow solid). LC/MS (ESI) m/z: 1175.0 [M+H]⁺. ¹H NMR (400 MHz, DMSO-d₆) δ 9.10 (s, 1H), 8.98 (s, 1H), 8.57 (s, 1H), 8.47 (d, J=7.6 Hz, 1H), 8.00 (d, J=9.6 Hz, 1H), 7.85-7.79 (m, 1H), 7.66 (d, J=8.0 Hz, 1H), 7.46-7.33 (m, 5H), 7.28 (d, J=2.4 Hz, 1H), 7.12 (d, J=7.2 Hz, 1H), 6.97 (d, J=2.4 Hz, 1H), 4.91 (quin, J=7.2 Hz, 1H), 4.66 (br d, J=9.8 Hz, 1H), 4.53-4.41 (m, 6H), 4.32-4.19 (m, 5H), 3.76-3.60 (m, 10H), 2.95 (br d, J=10.4 Hz, 2H), 2.70 (br t, J=5.6 Hz, 2H), 2.47-2.41 (m, 3H), 2.30-2.16 (m, 2H), 2.10-1.96 (m, 3H), 1.76-1.67 (m, 4H), 1.57 (br s, 4H), 1.38 (d, J=7.2 Hz, 3H), 1.24 (br dd, J=3.6, 6.4 Hz, 1H), 1.14 (q, J=10.4 Hz, 2H), 0.97 (s, 9H), 0.81 (t, J=7.2 Hz, 3H).

Exemplary Synthesis of (2S,4R)-1-[(2R)-2-[3-[7-[2-[7-(8-ethyl-3-hydroxy-1-naphthyl)-8-fluoro-4-(8-formyl-3,8-diazabicyclo[3.2.1]octan-3-yl)pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 290)

Step 1: Preparation of 3-(7-chloro-8-fluoro-2-methylsulfanyl-pyrido[4,3-d]pyrimidin-4-yl)-3,8-diazabicyclo[3.2.1]octane-8-carbaldehyde

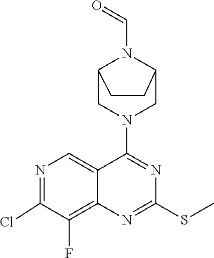 [SMILES: CSc1nc(N2CC3CCC(C2)N3C=O)c2cnc(Cl)c(F)c2n1]

To a solution of DCC (4.69 g, 22.7 mmol, 2 eq) in CHCl₃ (40 mL) at 0° C. was added a solution of HCOOH (2.18 g, 45.5 mmol, 4 eq) in CHCl₃ (10 mL) dropwise, and the resulting mixture was stirred for 5 minutes. A solution of 4-((1R,5S)-3,8-diazabicyclo[3.2.1]octan-3-yl)-7-chloro-8-fluoro-2-(methylthio)pyrido[4,3-d]pyrimidine (3.81 g, 11.3 mmol, FA salt) in pyridine (25 mL) was then added, and the reaction mixture was stirred at 25° C. for 16 hours. The mixture was concentrated, then diluted with EtOAc (50 mL). The resulting precipitate was removed by filtration and washed with EtOAc (20 mL). The combined organic extracts were evaporated to give a residue, which was purified by flash chromatography on SiO₂ (gradient: 0˜100% ethyl acetate in petroleum ether) to afford 955 mg of product as a yellow solid. The remaining solid from the filtration step was taken up in CH₂Cl₂ (200 mL), and the resulting suspension was stirred at 25° C. for 2 hours, then filtered. The filtrate was concentrated under reduced pressure to give 3-(7-chloro-8-fluoro-2-methylsulfanyl-pyrido[4,3-d]pyrimidin-4-yl)-3,8-diazabicyclo[3.2.1]octane-8-carbaldehyde (2.81 g, 7.61 mmol, 68% yield) as a yellow solid. LC/MS (ESI) m/z: 367.8 [M+H]⁺.

Step 2: Preparation of tert-butyl 3-[7-[8-ethyl-3-(methoxymethoxy)-1-naphthyl]-8-fluoro-2-[[(2S,5S)-5-[[3-[[5-[(1S)-1-[(2S,4R)-4-hydroxy-2-[[(1S)-1-[4-(4-methylthiazol-5-yl)phenyl]ethyl]carbamoyl

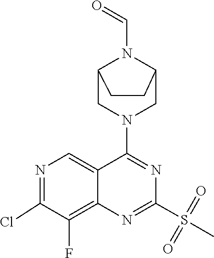 [SMILES: CS(=O)(=O)c1nc(N2CC3CCC(C2)N3C=O)c2cnc(Cl)c(F)c2n1]

To a solution of 3-(7-chloro-8-fluoro-2-methylsulfanyl-pyrido[4,3-d]pyrimidin-4-yl)-3,8-diazabicyclo[3.2.1]octane-8-carbaldehyde (2.81 g, 7.61 mmol, 1 eq) in DMF (30 mL) was added 4 Å MS (3.0 g), and the resulting mixture was stirred at 35° C. for 2 hours. Oxone (14.04 g, 22.84 mmol, 3 eq) was then added, and the reaction mixture was stirred at 35° C. for 16 hours. The reaction mixture was diluted with CH₂Cl₂ (50 mL) and then filtered. The filtrate was poured into water (60 mL) and extracted with CH₂Cl (50 mL×3). The combined organic extract was washed with water (80 mL×2), brine (80 mL×2), dried over anhydrous Na₂SO₄, filtered, and concentrated under reduced pressure. The crude product was triturated with ethyl acetate/petroleum ether (30 mL, V/V=1/2) at 20° C. for 15 minutes, and the resulting suspension was filtered and the cake was dried to give 3-(7-chloro-8-fluoro-2-methylsulfonyl-pyrido[4,3-d]pyrimidin-4-yl)-3,8-diazabicyclo[3.2.1]octane-8-carbaldehyde (1.60 g, crude) as a yellow solid. LC/MS (ESI) m/z: 399.8 [M+H]⁺.

Step 3: Preparation of 3-[7-chloro-2-(2,2-dimethoxyethoxy)-8-fluoro-pyrido[4,3-d]pyrimidin-4-yl]-3,8-diazabicyclo[3.2.1]octane-8-carbaldehyde

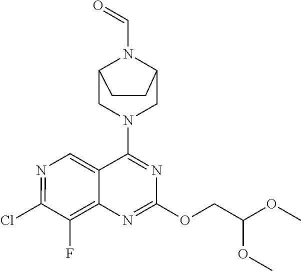 [SMILES: COC(COc1nc(N2CC3CCC(C2)N3C=O)c2cnc(Cl)c(F)c2n1)OC]

To a solution of 3-(7-chloro-8-fluoro-2-methylsulfonyl-pyrido[4,3-d]pyrimidin-4-yl)-3,8-diazabicyclo[3.2.1]octane-8-carbaldehyde (500 mg, 1.25 mmol, 1 eq) and 2,2-dimethoxyethanol (133 mg, 1.25 mmol, 1 eq) in toluene (7.5 mL) was added 4 Å MS (500 mg), and the resulting mixture was stirred at 20° C. for 10 minutes. t-BuONa (240 mg, 2.50 mmol, 2 eq) was then added at 0° C., and the reaction mixture was stirred at 0° C. for 0.5 hour. The reaction mixture was quenched by addition of water/2N aqueous HCl (10 mL/1 mL) and extracted with EtOAc (10 mL×3). The combined organic extract was washed with brine (20 mL), dried over anhydrous Na₂SO₄, filtered, and concentrated under reduced pressure. The crude product was purified by flash chromatography on SiO₂ (gradient: 0˜50% tetrahydrofuran in petroleum ether) to afford 3-[7-chloro-2-(2,2-dimethoxyethoxy)-8-fluoro-pyrido[4,3-d]pyrimidin-4-yl]-3,8-diazabicyclo[3.2.1]octane-8-carbaldehyde (1.37 g, 3.22 mmol, 86% yield) as a white solid. LC/MS (ESI) m/z: 425.9 [M+H]⁺.

Step 4: Preparation of 3-[2-(2,2-dimethoxyethoxy)-7-[8-ethyl-3-(methoxymethoxy)-1-naphthyl]-8-fluoro-pyrido[4,3-d]pyrimidin-4-yl]-3,8-diazabicyclo[3.2.1]octane-8-carbaldehyde

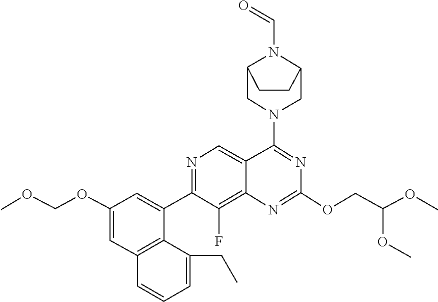

To a solution of 3-[7-chloro-2-(2,2-dimethoxyethoxy)-8-fluoro-pyrido[4,3-d]pyrimidin-4-yl]-3,8-diazabicyclo[3.2.1]octane-8-carbaldehyde (360 mg, 0.845 mmol, 1 eq) and 2-[8-ethyl-3-(methoxymethoxy)-1-naphthyl]-4,4,5,5-tetramethyl-1,3,2-dioxaborolane (318 mg, 0.930 mmol, 1.1 eq) in dioxane (8 mL) were added aq. K₃PO₄ (3 M, 0.845 mL, 3 eq) and CataCXium® A Pd G3 (62 mg, 0.085 mmol, 0.1 eq), and the reaction mixture was stirred at 85° C. under N₂ (degassed under vacuum and purged with N₂ several times) for 16 hours. The reaction mixture was quenched by addition of water (70 mL) and extracted with EtOAc (70 mL×3). The combined organic extract was washed with brine (70 mL), dried over anhydrous Na₂SO₄, filtered, and concentrated under reduced pressure. The crude product was purified by flash chromatography on SiO₂ (gradient: 0˜50% tetrahydrofuran in petroleum ether) to afford 3-[2-(2,2-dimethoxyethoxy)-7-[8-ethyl-3-(methoxymethoxy)-1-naphthyl]-8-fluoro-pyrido[4,3-d]pyrimidin-4-yl]-3,8-diazabicyclo[3.2.1]octane-8-carbaldehyde (368 mg, 0.608 mmol, 72% yield) as a yellow solid. LC/MS (ESI) m/z: 606.0 [M+H]⁺.

Step 5: Preparation of 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carbaldehyde

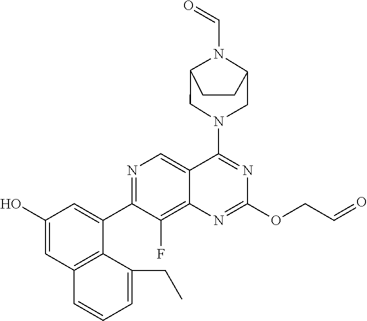 [SMILES: CCc1cccc2cc(O)cc(-c3ncc4c(N5CC6CCC(C5)N6C=O)nc(OCC=O)nc4c3F)c12]

To a stirred solution of 3-[2-(2,2-dimethoxyethoxy)-7-[8-ethyl-3-(methoxymethoxy)-1-naphthyl]-8-fluoro-pyrido[4,3-d]pyrimidin-4-yl]-3,8-diazabicyclo[3.2.1]octane-8-carbaldehyde (368 mg, 0.608 mmol, 1 eq) in acetone (1 mL) was added HCl (12 M, 1 mL), and the reaction mixture was stirred at 25° C. for 10 minutes. The mixture was poured into a solution of NaHCO₃ (1.53 g, 18.2 mmol, 30 eq) in water (20 mL)/THF (15 mL), and the resulting mixture was extracted with EtOAc (10 mL×3). The combined organic extract was dried over anhydrous Na₂SO₄, filtered, and concentrated under reduced pressure to give 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carbaldehyde (303 mg, crude) as a yellow solid. LC/MS (ESI) m/z: 516.0 [M+H]⁺.

Step 6: Preparation of (2S,4R)-1-[(2R)-2-[3-[7-[2-[7-(8-ethyl-3-hydroxy-1-naphthyl)-8-fluoro-4-(8-formyl-3,8-diazabicyclo[3.2.1]octan-3-yl)pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

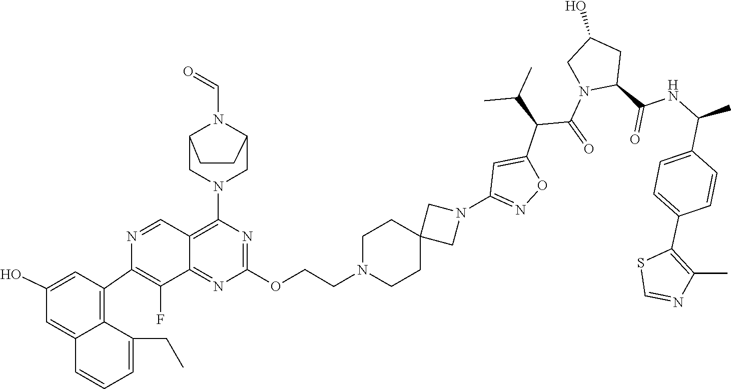 [SMILES: CCc1cccc2cc(O)cc(-c3ncc4c(N5CC6CCC(C5)N6C=O)nc(OCCN5CCC6(CC5)CN(c5cc([C@H](C(=O)N7C[C@H](O)C[C@H]7C(=O)N[C@@H](C)c7ccc(-c8scnc8C)cc7)C(C)C)on5)C6)nc4c3F)c12]

To a solution of 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carbaldehyde (153 mg, 0.297 mmol, 1.2 and (2S,4R)-1-[(2R)-2-[3-(2,7-diazaspiro[3.5]nonan-2-yl)isoxazol-5-yl]-3-methyl-eq) butanoyl]-4-hydroxy-N-[(1S)-1-[4-(4-methylthiazol-5-yl)phenyl]ethyl]pyrrolidine-2-carboxamide (150 mg, 0.247 mmol, 1 eq) in CH₂Cl₂ (5 mL)/isopropanol (5 mL) were added AcOH (59 mg, 0.99 mmol, 57 μL, 4 eq) and 2-picoline borane (106 mg, 0.989 mmol, 4 eq), and the reaction mixture was stirred at 25° C. under N₂ for 2 hours. The mixture was concentrated under reduced pressure, and the crude product was purified by flash chromatography on SiO₂ (gradient: 0˜10% methanol in dichloromethane) followed by prep-HPLC (column: Xtimate C18 150*40 mm*10 um; mobile phase: [0-40% CH₃CN in water (formic acid)]). Pure fractions were combined and concentrated under reduced pressure, then lyophilized to afford (2S,4R)-1-[(2R)-2-[3-[7-[2-[7-(8-ethyl-3-hydroxy-1-naphthyl)-8-fluoro-4-(8-formyl-3,8-diazabicyclo[3.2.1]octan-3-yl)pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (80.9 mg, 0.073 mmol, 29% yield, formic acid salt) as a white solid. LC/MS (ESI) m/z: 1106.2 [M+H]⁺. ¹H NMR (400 MHz, CD₃OD) δ 9.07 (s, 1H), 8.88 (s, 1H), 8.26 (s, 1H), 7.63 (d, J=8.0 Hz, 1H), 7.48-7.33 (m, 5H), 7.29 (d, J=2.4 Hz, 1H), 7.17 (d, J=7.2 Hz, 1H), 7.01 (d, J=2.8 Hz, 1H), 5.89-5.81 (m, 1H), 5.03 (q, J=6.8 Hz, 1H), 4.84-4.79 (m, 1H), 4.77-4.72 (m, 3H), 4.65-4.60 (m, 2H), 4.50 (t, J=8.4 Hz, 1H), 4.46-4.37 (m, 2H), 3.86-3.68 (m, 7H), 3.66-3.49 (m, 2H), 3.18-3.05 (m, 2H), 2.96-2.77 (m, 3H), 2.51-2.45 (m, 3H), 2.40-2.15 (m, 4H), 2.08-1.85 (m, 9H), 1.61-1.48 (m, 3H), 1.04 (d, J=6.4 Hz, 3H), 0.94-0.83 (m, 6H).

Exemplary Synthesis of Preparation of (2S,4R)-1-[(2R)-2-[3-[[3-[[1-[2-[7-(8-ethyl-3-hydroxy-1-naphthyl)-8-fluoro-4-(8-formyl-3,8-diazabicyclo[3.2.1]octan-3-yl)pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (Compound 246)

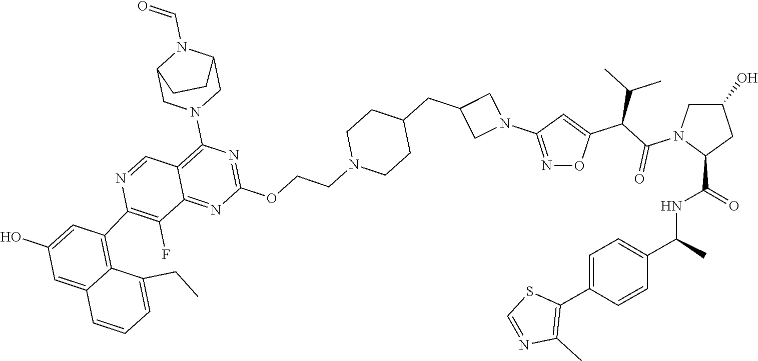 [SMILES: CCc1cccc2cc(O)cc(-c3ncc4c(N5CC6CCC(C5)N6C=O)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was made in an analogous manner to (2S,4R)-1-[(2R)-2-[3-[7-[2-[7-(8-ethyl-3-hydroxy-1-naphthyl)-8-fluoro-4-(8-formyl-3,8-diazabicyclo[3.2.1]octan-3-yl)pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide starting from (2S,4R)-4-hydroxy-1-[(2R)-3-methyl-2-[3-[[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide and 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carbaldehyde. LC/MS (ESI) m/z: 1134.9 [M+H]⁺. ¹H NMR (400 MHz, DMSO-d₆) δ 9.92 (s, 1H), 9.09 (s, 1H), 8.98 (s, 1H), 8.41 (d, J=7.6 Hz, 1H), 8.21 (s, 1H), 7.67 (d, J=8.4 Hz, 1H), 7.49-7.31 (m, 5H), 7.28 (d, J=2.4 Hz, 1H), 7.12 (d, J=7.2 Hz, 1H), 6.96 (d, J=2.4 Hz, 1H), 5.82-5.69 (m, 1H), 5.13-5.00 (m, 1H), 4.90 (quin, J=7.2 Hz, 1H), 4.69-4.52 (m, 3H), 4.51-4.43 (m, 2H), 4.38-4.25 (m, 2H), 3.98-3.89 (m, 2H), 3.76-3.50 (m, 4H), 3.47-3.38 (m, 5H), 2.91 (br d, J=9.6 Hz, 2H), 2.71-2.64 (m, 2H), 2.45 (s, 3H), 2.31-2.15 (m, 3H), 2.04-1.72 (m, 8H), 1.59-1.47 (m, 4H), 1.45-1.34 (m, 3H), 1.21-1.04 (m, 3H), 0.98-0.89 (m, 3H), 0.85-0.74 (m, 6H).

Exemplary Synthesis of (2S,4R)-1-[(2S)-2-[3-[[3-[[1-[2-[7-(8-ethyl-3-hydroxy-1-naphthyl)-8-fluoro-4-(8-formyl-3,8-diazabicyclo[3.2.1]octan-3-yl)pyrido[4,3-d]pyrimidin-2-yl]oxyethyl]-4-piperidyl]methyl]azetidin-1-yl]isoxazol-5-yl]-3-methyl-butanoyl]-4-(Compound 247)

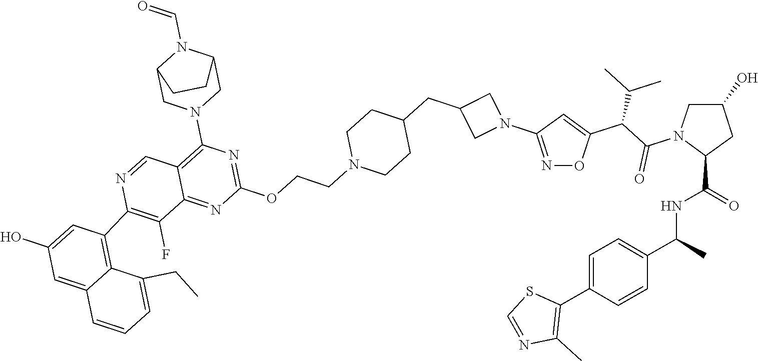 [SMILES: CCc1cccc2cc(O)cc(-c3ncc4c(N5CC6CCC(C5)N6C=O)nc(OCCN5CCC(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was made in an analogous manner to (2S,4R)-1-[(2R)-2-[3-[7-[2-[7-(8-ethyl-3-hydroxy-1-naphthyl)-8-fluoro-4-(8-formyl-3,8-diazabicyclo[3.2.1]octan-3-yl)pyrido[4,3-d]pyrimidin-2-yl]oxyethyl]-2,7-diazaspiro[3.5]nonan-2-yl]isoxazol-5-yl]-3-methyl-butanoyl]-4-hydroxy-N-[(1S)-1-[4-(4-methylthiazol-5-yl)phenyl]ethyl]pyrrolidine-2-starting from (2S,4R)-4-hydroxy-1-[(2S)-3-methyl-2-[3-[[3-(4-carboxamide piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide and 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carbaldehyde. LC/MS (ESI) m/z: 1134.9 [M+H]⁺. ¹H NMR (400 MHz, DMSO-d₆) δ 9.09 (s, 1H), 9.03-8.87 (m, 1H), 8.17 (s, 1H), 7.67 (d, J=8.0 Hz, 1H), 7.51-7.25 (m, 6H), 7.12 (d, J=7.2 Hz, 1H), 6.96 (d, J=2.4 Hz, 1H), 5.86-5.81 (m, 1H), 4.87 (quin, J=7.2 Hz, 1H), 4.70-4.53 (m, 3H), 4.50-4.44 (m, 2H), 4.44-4.35 (m, 2H), 4.30-4.22 (m, 1H), 3.95-3.85 (m, 2H), 3.77-3.47 (m, 12H), 2.96-2.89 (m, 2H), 2.71-2.65 (m, 2H), 2.47-2.42 (m, 3H), 2.31-2.15 (m, 3H), 2.09-1.69 (m, 8H), 1.61-1.38 (m, 5H), 1.34 (d, J=7.2 Hz, 2H), 1.21-1.01 (m, 3H), 0.94 (d, J=6.4 Hz, 2H), 0.83-0.72 (m, 6H).

Exemplary Synthesis of 5-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]piperazin-1-yl}-N-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]pyrazine-2-carboxamide (Compound 155)

Step 1: Preparation of methyl 5-(4-tert-butoxycarbonylpiperazin-1-yl) pyrazine-2-carboxylate

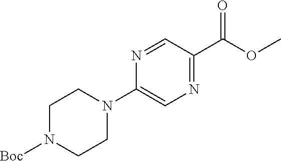 [SMILES: COC(=O)c1cnc(N2CCN(C(=O)OC(C)(C)C)CC2)cn1]

To a mixture of methyl 5-chloropyrazine-2-carboxylate (10 g, 58 mmol) and tert-butyl piperazine-1-carboxylate (11.9 g, 64 mmol) in N,N-dimethylformamide (100 mL) was added triethylamine (16.1 mL, 116 mmol) at 25° C. under nitrogen. The mixture was stirred at 80° C. for 3 h, poured into ice-water (w/w=1/1, 500 mL) and stirred for 5 min. The aqueous phase was extracted with ethyl acetate (200 mL×3). The combined organic phase was washed with brine (300 mL×2), dried with anhydrous sodium sulfate, filtered, and concentrated in vacuum. The crude product was triturated with t-butyl methyl ether (200 mL) at 30° C. for 15 min, filtered, the filter cake was collected to afford methyl 5-(4-tert-butoxycarbonylpiperazin-1-yl) pyrazine-2-carboxylate (16.8 g, 89%) as a yellow solid. MS (ESI) m/z: 323.1 [M+1]⁺; 1H NMR (400 MHz, DMSO-d₆) δ 8.66 (s, 1H), 8.36 (s, 1H), 3.81 (s, 3H), 3.76-3.69 (m, 4H), 3.50-3.41 (m, 4H), 1.42 (s, 9H).

Step 2: Preparation of 5-(4-tert-butoxycarbonylpiperazin-1-yl) pyrazine-2-carboxylic acid

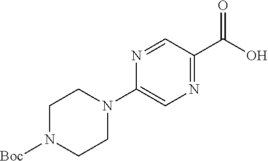 [SMILES: CC(C)(C)OC(=O)N1CCN(c2cnc(C(=O)O)cn2)CC1]

To a stirred suspension of methyl 5-(4-tert-butoxycarbonylpiperazin-1-yl) pyrazine-2-carboxylate (2 g, 6 mmol) in methanol (10 mL), tetrahydrofuran (10 mL) and water (20 mL) is added sodium hydroxide (1.24 g, 31 mmol) at 25° C. The mixture was stirred at 25° C. for 16 h, cooled to 0° C. and adjusted the pH to 3 with acetic acid. The aqueous phase was extracted with ethyl acetate (50 mL×3). The combined organic phase dried with anhydrous sodium sulfate, filtered, and concentrated in vacuum. The residue was purified by prep-HPLC (column: Phenomenex Luna C18 (250×70 mm, 10 um); mobile phase: [water (formic acid)-acetonitrile]; B %: 10%-40%, 20 min) to afford 5-(4-tert-butoxycarbonyl piperazin-1-yl) pyrazine-2-carboxylic acid (1.6 g, 84%) as a white solid. MS (ESI) m/z: 309.4 [M+1]⁺.

Step 3: Preparation of tert-butyl 4-[[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazine-1-carboxylate

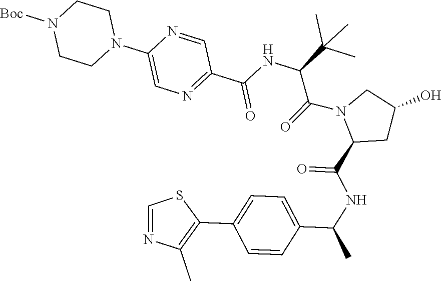 [SMILES: Cc1ncsc1-c1ccc([C@H](C)NC(=O)[C@@H]2C[C@@H](O)CN2C(=O)[C@@H](NC(=O)c2cnc(N3CCN(C(=O)OC(C)(C)C)CC3)cn2)C(C)(C)C)cc1]

To a mixture of 5-(4-tert-butoxycarbonylpiperazin-1-yl) pyrazine-2-carboxylic acid (1.6 g, 5.2 mmol) and (1R,4S)-2-[[(2S)-2-amino-3,3-dimethyl-butanoyl]-4-hydroxy-N-[(1S)-1-[4-(4-methylthiazol-5-yl)phenyl]ethyl]cyclopentanecarboxamide (2.3 g, 5.2 mmol) in N,N-dimethylformamide (70 mL) was added O-(7-Azabenzotriazol-1-yl)-N,N,N′,N′-tetramethyluronium Hexafluorophosphate (2.96 g, 7.8 mmol) and triethylamine (2.2 mL, 15.6 mmol) at 25° C. under nitrogen. The mixture was stirred at 25° C. for 1 h, then poured into ice-water (w/w=1/1, 500 mL) and stirred for 5 min. The aqueous phase was extracted with ethyl acetate (200 mL×3). The combined organic phase was washed with brine (200 mL×3), dried with anhydrous sodium sulfate, filtered, and concentrated in vacuum. The residue was purified by prep-HPLC (column: Welch Ultimate XB-SiOH 250×70, 10 um; mobile phase: [Hexane-ethanol]; B %: 15%-55%, 15 min) to afford tert-butyl 4-[[5-[(1S)-1-[(2S,4R)-4-hydroxy-2-thiazol-5-yl)phenyl]ethyl]carbamoyl]pyrrolidine-1-carbonyl]-2,2-[[(1S)-1-[4-(4-methyl dimethyl-propyl]carbamoyl]pyrazin-2-yl]piperazine-1-carboxylate (3.42 g, 89%) as a white solid. MS (ESI) m/z: 735.4 [M+1]⁺.

Step 4: Preparation of (2S,4R)-1-[(2R)-2-[3-[[3-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

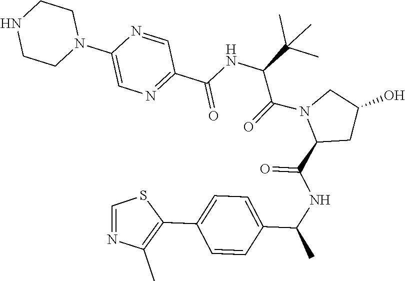 [SMILES: Cc1ncsc1-c1ccc([C@H](C)NC(=O)[C@@H]2C[C@@H](O)CN2C(=O)[C@@H](NC(=O)c2cnc(N3CCNCC3)cn2)C(C)(C)C)cc1]

To a solution of tert-butyl 4-[[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazine-1-carboxylate ((800 mg, 1.1 mmol) in dichloromethane (8 mL) was added trifluoroacetic acid (2 mL), the reaction solution was stirred at 25° C. for 1 h. The solution was concentrated under vacuum to afford N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]-5-piperazin-1-yl-pyrazine-2-carboxamide (820 mg, crude, trifluoroacetate) as a light yellow gum, which was used in the next step without further purification. MS (ESI) m/z: 635.2 [M+1]⁺.

Step 5: Preparation of tert-butyl 4-fluoro-4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]piperidine-1-carboxylate

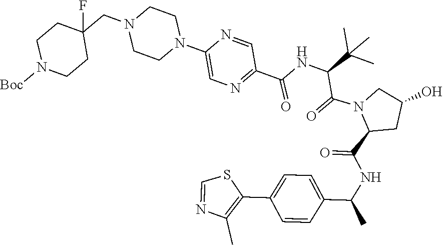 [SMILES: Cc1ncsc1-c1ccc([C@H](C)NC(=O)[C@@H]2C[C@@H](O)CN2C(=O)[C@@H](NC(=O)c2cnc(N3CCN(CC4(F)CCN(C(=O)OC(C)(C)C)CC4)CC3)cn2)C(C)(C)C)cc1]

To a solution of N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]-5-piperazin-1-yl-pyrazine-2-carboxamide (720 mg, 1 mmol, trifluoroacetate) in methanol (8 mL) was added triethylamine (0.3 mL, 2 mmol), then tert-butyl 4-fluoro-4-formyl-piperidine-1-carboxylate (1.11 g, 4.8 mmol) and acetic acid (58 mg, 0.1 mmol) was added. The solution was stirred at 25° C. for 0.5 h. To the solution was added sodium cyanoborohydride (181 mg, 2.9 mmol) and stirred at 25° C. for 4 h. To the reaction was added water (20 mL) and extracted with ethyl acetate (20 mL×2). The combined organic layer was washed with brine (10 mL×2). The organic layer was dried over anhydrous sodium sulfate, filtered, and concentrated under vacuum. The residue was purified by silica gel column chromatography (petroleum ether/ethyl acetate=1/1 to dichloromethane/methanol=10/1) to afford tert-butyl 4-fluoro-4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]piperidine-1-carboxylate (530 mg, 64%) as a yellow solid. MS (ESI) m/z: 850.3 [M+1]⁺.

Step 6: Preparation of 5-[4-[(4-fluoro-4-piperidyl)methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide

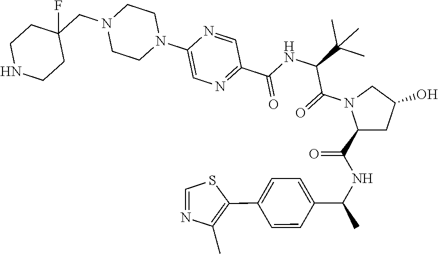 [SMILES: Cc1ncsc1-c1ccc([C@H](C)NC(=O)[C@@H]2C[C@@H](O)CN2C(=O)[C@@H](NC(=O)c2cnc(N3CCN(CC4(F)CCNCC4)CC3)cn2)C(C)(C)C)cc1]

To a solution of tert-butyl 4-fluoro-4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]piperidine-1-carboxylate (400 mg, 0.47 mmol) in dichloromethane (5 mL) was added trifluoroacetic acid (1.0 mL, 13 mmol), the reaction solution was stirred at 25° C. for 0.5 h. The solution was concentrated under vacuum to afford 5-[4-[(4-fluoro-4-piperidyl)methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide (400 mg, crude, trifluoroacetate) as a light yellow gum, which was used in the next step without further purification. MS (ESI) m/z: 750.4 [M+1]⁺.

Step 7: Preparation of tert-butyl 3-[7-[8-ethyl-3-(methoxymethoxy)-1-naphthyl]-8-fluoro-2-[2-[[4-fluoro-4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]-1-piperidyl]ethoxy]pyrido[4,3-d]pyrimidin-4-yl]-3,8-diazabicyclo[3.2.1]octane-8-carboxylate

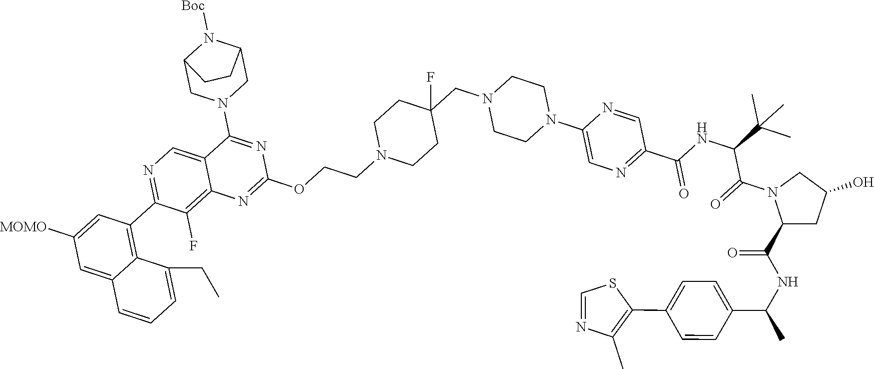

To a solution of 5-[4-[(4-fluoro-4-piperidyl)methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide (400 mg, 0.5 mmol, trifluoroacetate) in dichloromethane (4 mL) and isopropanol (4 mL) was added N,N-diisopropylethylamine (0.2 mL, 1 mmol), then tert-butyl 3-[7-[8-ethyl-3-(methoxymethoxy)-1-naphthyl]-8-fluoro-2-(2-oxoethoxy)pyrido[4,3-d]pyrimidin-4-yl]-3,8-diazabicyclo[3.2.1]octane-8-carboxylate (292 mg, 0.46 mmol) was added. The solution was stirred at 25° C. for 10 min. To the solution was added sodium triacetoxyborohydride (294 mg, 1.4 mmol), the reaction solution was stirred at 25° C. for 0.5 h, then diluted with water (30 mL) and extracted with dichloromethane (30 mL×3). The combined organic layer was dried over anhydrous sodium sulfate, filtered, and concentrated under vacuum. The residue was purified by prep-HPLC (column: Waters Xbridge Prep OBD C18 150×40 mm×10 um; mobile phase: [water (ammonium bicarbonate)-acetonitrile]; B %: 60%-90%, 10 min) to afford tert-butyl 3-[7-[8-ethyl-3-(methoxymethoxy)-1-naphthyl]-8-fluoro-2-[2-[[4-fluoro-4-[[4-[5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]-1-piperidyl]ethoxy]pyrido[4,3-d]pyrimidin-4-yl]-3,8-diazabicyclo[3.2.1]octane-8-carboxylate (470 mg, 74%) as a light yellow solid. MS (ESI) m/z: 1165.8 [M+1]⁺; ¹H NMR (400 MHz, DMSO-d₆) δ 9.15 (s, 1H), 8.99 (s, 1H), 8.62 (s, 1H), 8.47 (br d, J=7.6 Hz, 1H), 8.32 (s, 1H), 8.02 (br d, J=9.6 Hz, 1H), 7.80 (d, J=8.0 Hz, 1H), 7.62 (s, 1H), 7.51-7.41 (m, 3H), 7.40-7.33 (m, 2H), 7.24 (d, J=7.2 Hz, 1H), 7.16 (d, J=1.2 Hz, 1H), 5.35 (s, 2H), 5.15 (d, J=3.2 Hz, 1H), 4.92 (quin, J=6.8 Hz, 1H), 4.68 (br d, J=10.0 Hz, 1H), 4.62-4.41 (m, 5H), 4.31 (br s, 3H), 3.76-3.55 (m, 8H), 3.43 (s, 3H), 3.32 (s, 2H), 2.79-2.66 (m, 4H), 2.57 (br d, J=11.2 Hz, 5H), 2.47-2.42 (m, 3H), 2.40-2.17 (m, 4H), 2.12-2.02 (m, 1H), 1.95-1.78 (m, 5H), 1.76-1.58 (m, 4H), 1.47 (s, 9H), 1.39 (br d, J=6.8 Hz, 2H), 0.99 (s, 9H), 0.84 (t, J=7.2 Hz, 3H).

Step 8: Preparation of 5-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide

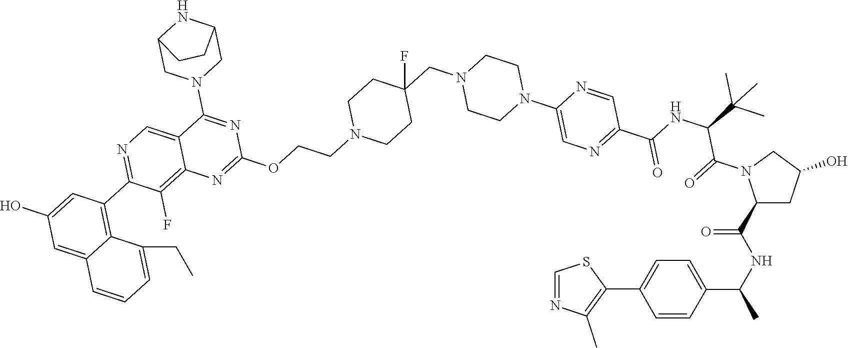 [SMILES: CCc1cccc2cc(O)cc(-c3ncc4c(N5CC6CCC(C5)N6)nc(OCCN5CCC(F)(CN6CCN(c7cnc(C(=O)N[C@H](C(=O)N8C[C@H](O)C[C@H]8C(=O)N[C@@H](C)c8ccc(-c9scnc9C)cc8)C(C)(C)C)cn7)CC6)CC5)nc4c3F)c12]

To a solution of tert-butyl 3-[7-[8-ethyl-3-(methoxymethoxy)-1-naphthyl]-8-fluoro-2-[2-[[4-fluoro-4-[[4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]pyrazin-2-yl]piperazin-1-yl]methyl]-1-piperidyl]ethoxy]pyrido[4,3-d]pyrimidin-4-yl]-3,8-diazabicyclo[3.2.1]octane-8-carboxylate (470 mg, 0.3 mmol) in dichloromethane (8 mL) was added hydrogen chloride/dioxane (4 M, 8 mL), the reaction mixture was stirred at 25° C. for 15 min. The mixture was diluted with petroleum ether (25 mL) and filtered. The filtered cake was for purification. The residue was purified by prep-HPLC (column: Waters Xbridge Prep OBD C18 150×40 mm×10 um; mobile phase: [water (NH₃H₂O)-acetonitrile]; B %: 40%-70%, 10 min) to afford 5-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]piperazin-1-yl]-N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]pyrazine-2-carboxamide (255 mg, 58%) as an off-white solid. MS (ESI) m/z: 1221.7 [M+1]⁺; 1H NMR (400 MHz, DMSO-d₆) δ 9.89 (br s, 1H), 9.09 (s, 1H), 9.03-8.94 (m, 1H), 8.61 (d, J=1.2 Hz, 1H), 8.46 (d, J=8.0 Hz, 1H), 8.32 (s, 1H), 8.08-7.90 (m, 1H), 7.66 (d, J=8.0 Hz, 1H), 7.47-7.42 (m, 2H), 7.41-7.32 (m, 3H), 7.28 (d, J=2.8 Hz, 1H), 7.12 (d, J=6.8 Hz, 1H), 6.97 (d, J=2.4 Hz, 1H), 5.14 (d, J=3.2 Hz, 1H), 5.03-4.82 (m, 1H), 4.75-4.63 (m, 1H), 4.56-4.35 (m, 5H), 4.33-4.21 (m, 1H), 3.77-3.59 (m, 7H), 3.58-3.40 (m, 3H), 2.78-2.65 (m, 4H), 2.65-2.52 (m, 7H), 2.46-2.43 (m, 3H), 2.37-2.29 (m, 2H), 2.28-2.14 (m, 2H), 2.06 (dd, J=7.2, 12.8 Hz, 1H), 1.87-1.70 (m, 4H), 1.69-1.56 (m, 5H), 1.38 (d, J=7.2 Hz, 3H), 0.98 (s, 9H), 0.81 (t, J=7.6 Hz, 3H).

Exemplary Synthesis of (2S,4R)-1-[(2R)-2-(3-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 156)

Step 1: Preparation of tert-butyl 4-[[1-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]piperidine-1-carboxylate

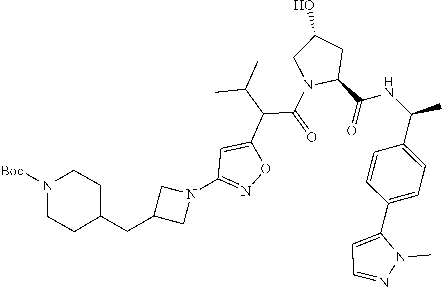 [SMILES: CC(C)C(C(=O)N1C[C@H](O)C[C@H]1C(=O)N[C@@H](C)c1ccc(-c2ccnn2C)cc1)c1cc(N2CC(CC3CCN(C(=O)OC(C)(C)C)CC3)C2)no1]

To a mixture of 2-[3-[[3-[(1-tert-butoxycarbonyl-4-piperidyl)methyl]azetidin-1-yl]isoxazol-5-yl]-3-methyl-butanoic acid (722 mg, 1.7 mmol) and (2S,4R)-4-hydroxy-N-[(1S)-1-[4-(2-methylpyrazol-3-yl)phenyl]ethyl]pyrrolidine-2-carboxamide hydrochloride (664 mg, 1.9 mmol) in DMF (10 mL) was added DIEA (1.5 mL, 8.6 mmol) and HATU (716 mg, 1.9 mmol), the resulting suspension was stirred at 25° C. for 15 h. The reaction mixture was diluted with water (20 mL) and ethyl acetate (20 mL). The organic layer was separated, and the aqueous layer was extracted with ethyl acetate (20 mL×3). The combined organic layer was washed with water (20 mL×2), brine (50 mL×2), dried over Na₂SO₄, filtered, and concentrated under reduced pressure. The residue was purified by column chromatography (0-4% methanol in dichloromethane) to afford tert-butyl 4-[[1-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]piperidine-1-carboxylate (616 mg, 46%) as brown solid. MS (ESI) m/z: 718.3 [M+H]⁺.

Step 2: Preparation of tert-butyl 4-[[1-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]piperidine-1-carboxylate and tert-butyl 4-[[1-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]piperidine-1-carboxylate

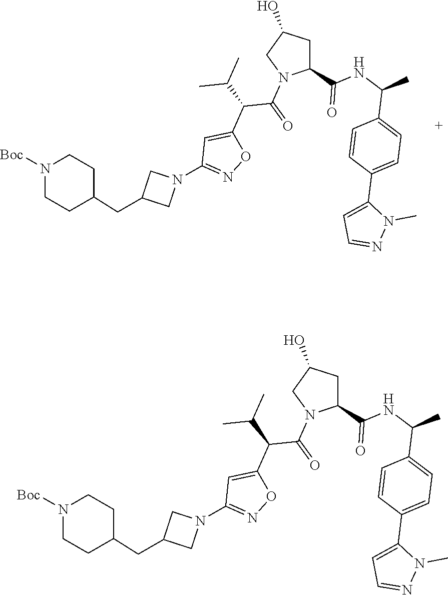 [SMILES: CC(C)[C@H](C(=O)N1C[C@H](O)C[C@H]1C(=O)N[C@@H](C)c1ccc(-c2ccnn2C)cc1)c1cc(N2CC(CC3CCN(C(=O)OC(C)(C)C)CC3)C2)no1 ; CC(C)[C@@H](C(=O)N1C[C@H](O)C[C@H]1C(=O)N[C@@H](C)c1ccc(-c2ccnn2C)cc1)c1cc(N2CC(CC3CCN(C(=O)OC(C)(C)C)CC3)C2)no1]

Racemic tert-butyl 4-[[1-[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]piperidine-1-carboxylate (616 mg) was separated by SFC (column: DAICEL CHIRALPAK AD (250 mm*50 mm, 10 um); mobile phase: [0.1% NH₃H₂O-IPA]; Begin B: 40% End B: 40%). Fraction I was obtained as tert-butyl 4-[[1-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]piperidine-1-carboxylate (309 mg, 51%) as an orange solid. Fraction II was obtained as tert-butyl 4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]piperidine-1-carboxylate (210 mg, 35%) as an orange solid. MS (ESI) m/z: 718.2 [M+H]⁺.

Step 3: Preparation of (2S,4R)-4-hydroxy-1-[(2R)-3-methyl-2-[3-[[3-(4-piperidylmethyl) azetidin-1-yl]isoxazol-5-yl]butanoyl]-N-[(1S)-1-[4-(2-methylpyrazol-3-yl)phenyl]ethyl]pyrrolidine-2-carboxamide

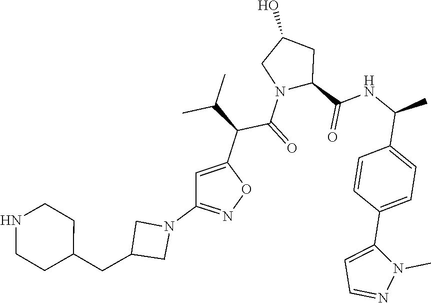 [SMILES: CC(C)[C@@H](C(=O)N1C[C@H](O)C[C@H]1C(=O)N[C@@H](C)c1ccc(-c2ccnn2C)cc1)c1cc(N2CC(CC3CCNCC3)C2)no1]

To a mixture of tert-butyl 4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]piperidine-1-carboxylate (210 mg, 0.3 mmol) in dichloromethane (3 mL) was added trifluoroacetic acid (1.5 mL), the reaction was stirred at 25° C. for 0.5 h, concentrated under reduced pressure. The pH of the residue was adjusted to 10 by saturated aqueous sodium carbonate and extracted with dichloromethane/methanol (30 mL×3, V:V=10:1). The combined organic layer was dried over Na₂SO₄, filtered, and concentrated under reduced pressure to afford (2S,4R)-4-hydroxy-1-[(2R)-3-methyl-2-[3-[[3-(4-piperidylmethyl) azetidin-1-yl]isoxazol-5-yl]butanoyl]-N-[(1S)-1-[4-(2-methylpyrazol-3-yl)phenyl]ethyl]pyrrolidine-2-carboxamide (169 mg, crude) as an orange solid, which was used in the next step directly. MS (ESI) m/z: 618.4 [M+H]⁺.

Step 4: Preparation of tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate

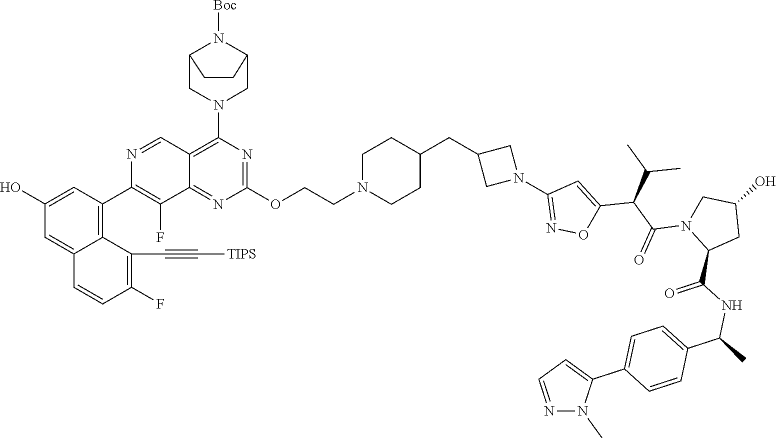

To a mixture of (2S,4R)-4-hydroxy-1-[(2R)-3-methyl-2-[3-[[3-(4-piperidylmethyl) azetidin-1-yl]isoxazol-5-yl]butanoyl]-N-[(1S)-1-[4-(2-methylpyrazol-3-yl)phenyl]ethyl]pyrrolidine-2-carboxamide (163 mg, 0.26 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (240 mg, 0.3 mmol) in CH₂Cl₂ (6 mL) and i-PrOH (1 mL) was added acetic acid (60 μL, 1 mmol) and 2-methylpyridine borane (141 mg, 1.3 mmol), the resulting suspension was stirred at 25° C. for 15 h, then concentrated under reduced pressure. The residue was purified by column chromatography (0-8% to 10% methanol in dichloromethane) to afford tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate (200 mg, 48%) as a yellow solid. MS (ESI) m/z: 1359.6 [M+H]⁺.

Step 5: Preparation of tert-butyl 3-[7-(8-ethynyl-7-fluoro-3-hydroxy-1-naphthyl)-8-fluoro-2-[2-[[4-[[1-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate

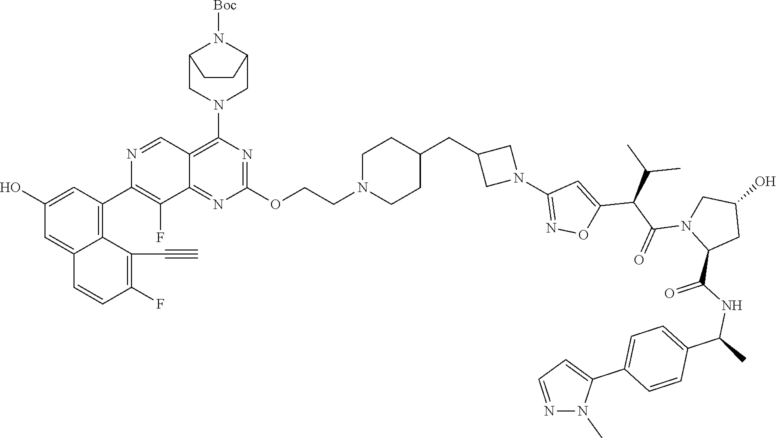 [SMILES: C#Cc1c(F)ccc2cc(O)cc(-c3ncc4c(N5CC6CCC(C5)N6C(=O)OC(C)(C)C)nc(OCCN5CCC(CC6CN(c7cc([C@H](C(=O)N8C[C@H](O)C[C@H]8C(=O)N[C@@H](C)c8ccc(-c9ccnn9C)cc8)C(C)C)on7)C6)CC5)nc4c3F)c12]

To a mixture of tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate (200 mg, 0.1 mmol) in THF (6 mL) was added TBAF (1.0 M, 252 uL), the resulting suspension was stirred at 25° C. for 4 h. The reaction mixture was diluted with ethyl acetate (50 mL) and washed with water (6 mL×3), brine (20 mL), dried over Na₂SO₄, filtered, and concentrated under reduced pressure to afford tert-butyl 3-[7-(8-ethynyl-7-fluoro-3-hydroxy-1-naphthyl)-8-fluoro-2-[2-[[4-[[1-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate (325 mg, crude) as a yellow solid, which was used in the next step directly. MS (ESI) m/z: 1203.4 [M+H]⁺.

Step 6: Preparation of (2S,4R)-1-[(2R)-2-[3-[[3-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

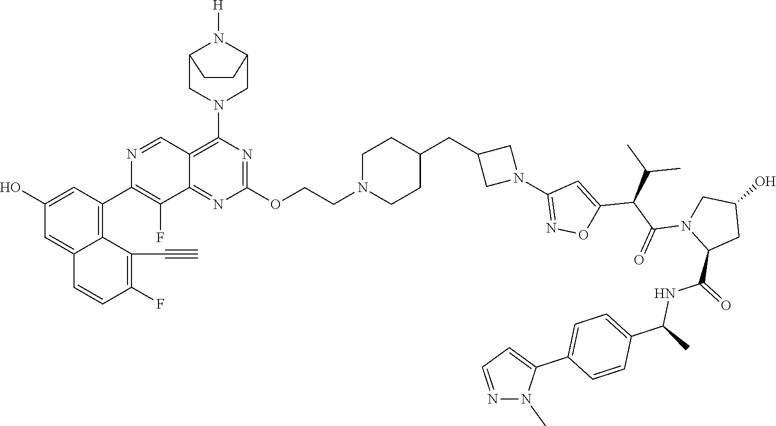 [SMILES: C#Cc1c(F)ccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9ccnn9C)cc8)C(C)C)on7)C6)CC5)nc4c3F)c12]

To a mixture of tert-butyl 3-[7-(8-ethynyl-7-fluoro-3-hydroxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate (151 mg, 0.1 mmol) in CH₂Cl₂ (4 mL) was added TFA (2.0 mL), the reaction was stirred at 25° C. for 0.5 h, then concentrated under reduced pressure. The residue was diluted with water (3 mL), adjusted the pH with sat. aq. Na₂CO₃ to 10 and extracted with dichloromethane/methanol (20 mL×3), dried over Na₂SO₄, filtered, and concentrated under reduced pressure. The crude product was purified by prep-HPLC (column: Xtimate C18 150*40 mm, 10 um; mobile phase: [water (ammonium bicarbonate)-acetonitrile]; B: 20-60; flow rate (ml/min): 25). The crude product was further purified by prep-HPLC (column: DAICEL CHIRALPAK IC 250 mm*30 mm, 10 um; mobile phase: [acetonitrile/ethanol (0.1% NH₃H₂O)]; flow rate (ml/min): 80) to afford (2S,4R)-1-[(2R)-2-[3-[[3-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (38.0 mg, 27%) as a light brown solid. MS (ESI) m/z: 1103.5 [M+H]⁺; ¹H NMR (400 MHz, CD₃OD) δ 9.01 (s, 1H), 7.86 (dd, J=5.6, 9.2 Hz, 1H), 7.50-7.38 (m, 5H), 7.37-7.28 (m, 2H), 7.21 (d, J=2.4 Hz, 1H), 6.34 (d, J=2.0 Hz, 1H), 5.85-5.78 (m, 1H), 5.08-4.99 (m, 1H), 4.66-4.54 (m, 6H), 4.51 (t, J=8.0 Hz, 1H), 4.46-4.39 (m, 1H), 4.03 (t, J=7.6 Hz, 2H), 3.85 (s, 3H), 3.74-3.52 (m, 8H), 3.37 (s, 1H), 3.08 (d, J=7.6 Hz, 2H), 2.87-2.81 (m, 2H), 2.40-2.29 (m, 1H), 2.23-2.10 (m, 3H), 1.95 (ddd, J=4.8, 8.4, 13.2 Hz, 1H), 1.90-1.76 (m, 4H), 1.68 (d, J=9.6 Hz, 2H), 1.62-1.58 (m, 2H), 1.52 (d, J=6.8 Hz, 3H), 1.33-1.25 (m, 3H), 1.06-1.02 (m, 3H), 0.93-0.86 (m, 3H).

Exemplary Synthesis of (2S,4R)-1-[(2S)-2-(2-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]-4-fluoropiperidin-1-yl}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide (Compound 157)

Step 1: Preparation of tert-butyl 3-[8-fluoro-2-[2-[[4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate

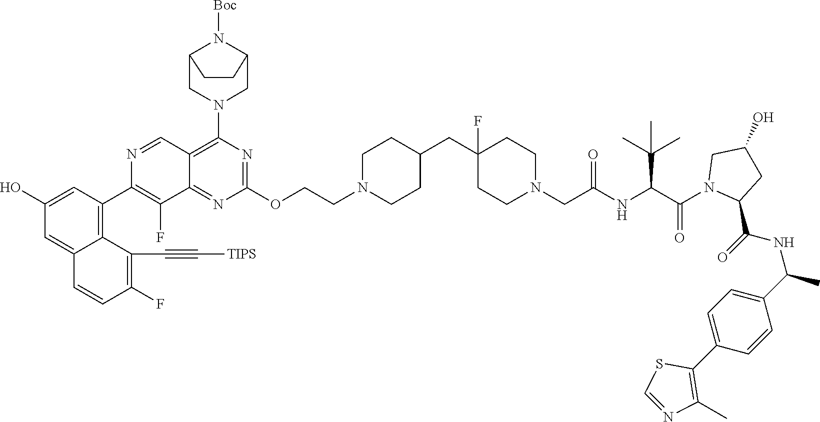

To a solution of (2S,4R)-1-[(2S)-2-[2-[[4-fluoro-4-(4-piperidylmethyl)-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (320 mg, 0.5 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (354 mg, 0.5 mmol) in dichloromethane (5.0 mL) and i-PrOH (5.0 mL) were added acetic acid (0.11 mL, 1.9 mmol) and 2-methylpyridine borane (200 mg, 1.9 mmol). The mixture was stirred at 25° C. for 1 h, then concentrated. The residue was purified by column chromatography (0-15% methanol in dichloromethane) to give tert-butyl 3-[8-fluoro-2-[2-[[4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (450 mg, 68%) as a yellow solid. MS (ESI) m/z: 1426.6 [M+1]⁺.

Step 2: Preparation of tert-butyl 3-[7-(8-ethynyl-7-fluoro-3-hydroxy-1-naphthyl)-8-fluoro-2-[2-[[4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate

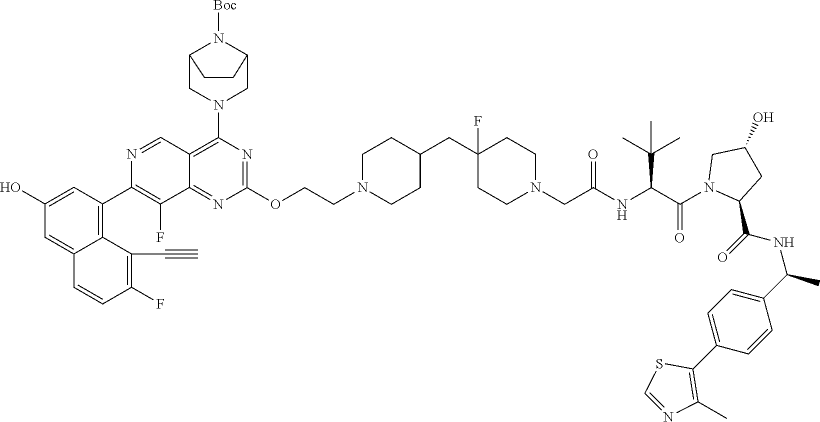 [SMILES: C#Cc1c(F)ccc2cc(O)cc(-c3ncc4c(N5CC6CCC(C5)N6C(=O)OC(C)(C)C)nc(OCCN5CCC(CC6(F)CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12]

To a solution of tert-butyl 3-[8-fluoro-2-[2-[[4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (450 mg, 0.3 mmol) in tetrahydrofuran (6.0 mL) was added TBAF (1 M, 1.3 mL), the mixture was stirred at 25° C. for 16 h. The reaction was diluted with ethyl acetate (50 mL) and washed with water (10 mL×10). The combined organic phase was dried over anhydrous sodium sulfate, filtered, and concentrated over vacuum to give tert-butyl 3-[7-(8-ethynyl-7-fluoro-3-hydroxy-1-naphthyl)-8-fluoro-2-[2-[[4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate (400 mg, 99.8%) as a yellow solid. MS (ESI) m/z: 1270.5 [M+1]⁺.

Step 3: Preparation of (2S,4R)-1-[(2S)-2-[[2-[[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]-4-fluoro-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

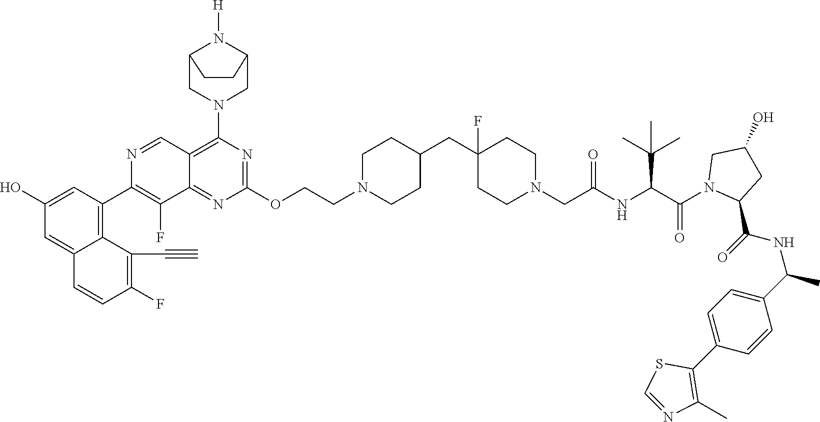 [SMILES: C#Cc1c(F)ccc2cc(O)cc(-c3ncc4c(N5CC6CCC(C5)N6)nc(OCCN5CCC(CC6(F)CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12]

To a solution of tert-butyl 3-[7-(8-ethynyl-7-fluoro-3-hydroxy-1-naphthyl)-8-fluoro-2-[2-[[4-[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate (400 mg, 0.3 mmol) in CH₂Cl₂ (3.0 mL) was added HCl/dioxane (4 M, 2.4 mL). The reaction mixture was stirred at 25° C. for 0.5 h, diluted with petroleum ether (40 mL) and filtered. The filter cake was dissolved with tetrahydrofuran (20 mL), adjusted the pH to 9. The mixture was filtered, and the filtrate was concentrated under reduced pressure. The crude product was purified by prep-HPLC (column: Xtimate C18 150*40 mm*10 um; mobile phase: [water (NH₄HCO₃)-ACN]; B %: 26%-66%, 25 min) to give (2S,4R)-1-[(2S)-2-[[2-[[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]-4-fluoro-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (140.2 mg, 38%) as a white solid. MS (ESI) m/z: 1170.3 [M+1]⁺; ¹H NMR (400 MHz, CD₃OD) δ 9.00 (s, 1H), 8.89-8.83 (m, 1H), 7.85 (J=5.6, 9.2 Hz, 1H), 7.47-7.37 (m, 4H), 7.36-7.28 (m, 2H), 7.21 (d, J=2.4 Hz, 1H), 5.03-4.97 (m, 1H), 4.66-4.54 (m, 6H), 4.43 (s, 1H), 3.90-3.81 (m, 1H), 3.77-3.63 (m, 5H), 3.37 (s, 1H), 3.13-3.02 (m, 4H), 2.89 (t, J=5.6 Hz, 2H), 2.73-2.61 (m, 2H), 2.52-2.42 (m, 5H), 2.30-2.16 (m, 3H), 1.98-1.75 (m, 11H), 1.67 (dd, J=4.4, 11.6 Hz, 2H), 1.61-1.57 (m, 1H), 1.53-1.47 (m, 3H), 1.35 (d, J=11.6 Hz, 2H), 1.07-0.99 (m, 9H).

Exemplary Synthesis of (2S,4R)-1-[(2S)-2-(3-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)oxy]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 158)

Step 1: Preparation of tert-butyl 3-[8-fluoro-7-[7-fluoro-3-hydroxy-8-(2-triisopropylsilylethynyl)-1-naphthyl]-2-[2-[[4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate

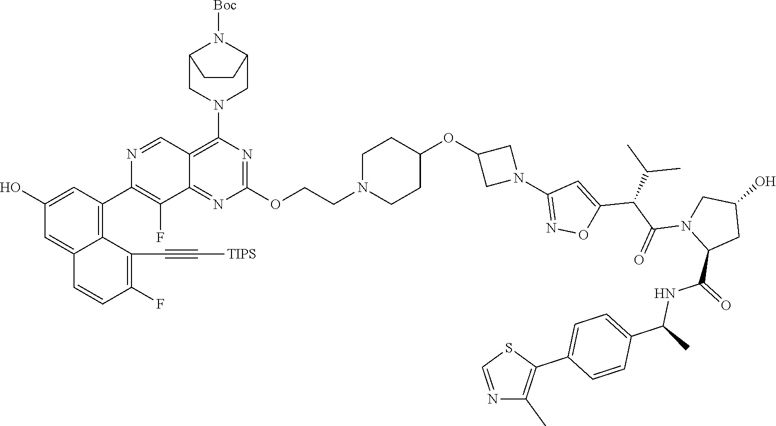

To a solution of (2S,4R)-4-hydroxy-1-[(2S)-3-methyl-2-[3-[[3-(4-piperidyloxy) azetidin-1-yl]isoxazol-5-yl]butanoyl]-N-[(1S)-1-[4-(4-methylthiazol-5-yl)phenyl]ethyl]pyrrolidine-2-carboxamide (300 mg, 0.5 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (357 mg, 0.5 mmol) in CH₂Cl₂ (5.0 mL) and i-PrOH (5.0 mL) were added acetic acid (0.1 mL, 2 mmol) and 2-methylpyridine borane (201 mg, 1.9 mmol). The mixture was stirred at 25° C. for 2 h, then concentrated. The crude product was purified by column chromatography (0-12% methanol in dichloromethane to give tert-butyl 3-[8-fluoro-7-[7-fluoro-3-hydroxy-8-(2-triisopropylsilylethynyl)-1-naphthyl]-2-[2-[[4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate (450 mg, 69%) as a yellow solid. MS (ESI) m/z: 1377.6 [M+1]⁺.

Step 2: Preparation of tert-butyl 3-[7-(8-ethynyl-7-fluoro-3-hydroxy-1-naphthyl)-8-fluoro-2-[2-[[4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate

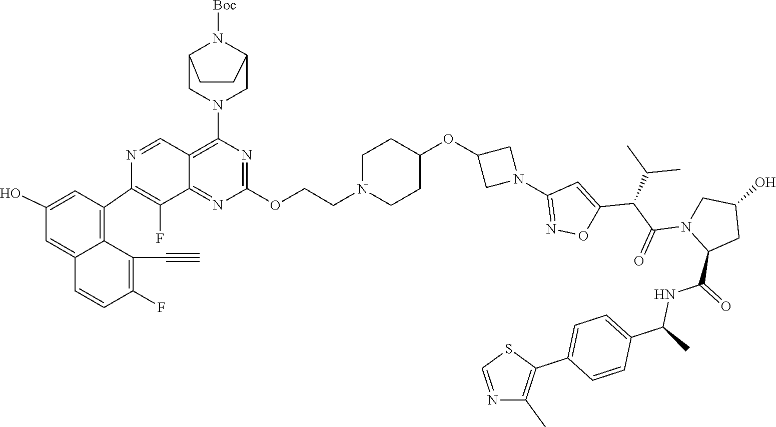 [SMILES: C#Cc1c(F)ccc2cc(O)cc(-c3ncc4c(N5CC6CCC(C5)N6C(=O)OC(C)(C)C)nc(OCCN5CCC(OC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12]

To a solution of tert-butyl 3-[8-fluoro-7-[7-fluoro-3-hydroxy-8-(2-triisopropylsilylethynyl)-1-naphthyl]-2-[2-[[4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate (450 mg, 0.3 mmol) in tetrahydrofuran (6.0 mL) was added TBAF (1 M, 1.3 mL), the reaction mixture was stirred at 25° C. for 16 h. The mixture was diluted with ethyl acetate (50 mL) and washed with water (10 mL×10). The combined organic phase was dried over anhydrous sodium sulfate, filtered, and concentrated in vacuum to give tert-butyl 3-[7-(8-ethynyl-7-fluoro-3-hydroxy-1-naphthyl)-8-fluoro-2-[2-[[4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate (390 mg, 98%) as a yellow solid. MS (ESI) m/z: 1222.5 [M+1]⁺.

Step 3: Preparation of (2S,4R)-1-[(2S)-2-[3-[[3-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]oxy]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

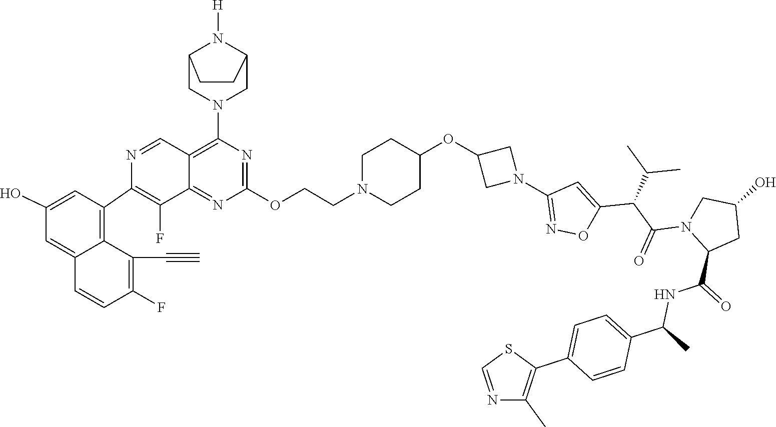 [SMILES: C#Cc1c(F)ccc2cc(O)cc(-c3ncc4c(N5CC6CCC(C5)N6)nc(OCCN5CCC(OC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12]

To a solution of tert-butyl 3-[7-(8-ethynyl-7-fluoro-3-hydroxy-1-naphthyl)-8-fluoro-2-[2-[[4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate (390 mg, 0.3 mmol) in CH₂Cl₂ (10.0 mL) was added TsOH·H₂O (607 mg, 3.2 mmol). The reaction mixture was stirred at 25° C. for 1 h, then concentrated under reduced pressure. The mixture was quenched with sat. aq. Na₂CO₃, adjusted the pH to 8, extracted with 10% methanol in dichloromethane (20 mL×3). The combined organic layer was washed with brine (5 mL), dried over anhydrous Na₂SO₄, filtered, and concentrated under reduced pressure. The crude product was purified by prep-HPLC (column: Xtimate C18 150*40 mm*10 um; mobile phase: [water (NH₄HCO₃)-ACN]; B %: 16%-56%, 25 min) to give (2S,4R)-1-[(2S)-2-[3-[[3-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]oxy]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (127.3 mg, 36%) as a white solid. MS (ESI) m/z: 1122.2 [M+1]⁺; 1H NMR (400 MHz, CD₃OD) δ 9.01 (s, 1H), 8.91-8.85 (m, 1H), 7.86 (dd, J=5.6, 9.2 Hz, 1H), 7.50-7.25 (m, 6H), 7.21 (d, J=2.0 Hz, 1H), 5.94-5.83 (m, 1H), 5.04-4.96 (m, 1H), 4.67-4.51 (m, 7H), 4.43 (s, 1H), 4.18-4.06 (m, 2H), 3.78-3.70 (m, 4H), 3.70-3.63 (m, 4H), 3.50-3.39 (m, 1H), 3.39-3.34 (m, 1H), 2.92 (d, J=4.8 Hz, 2H), 2.86 (t, J=5.6 Hz, 2H), 2.51-2.45 (m, 3H), 2.43-2.31 (m, 3H), 2.21 (d, J=3.2 Hz, 1H), 1.96 (td, J=4.0, 13.2 Hz, 1H), 1.91-1.75 (m, 6H), 1.66-1.58 (m, 2H), 1.58-1.46 (m, 3H), 1.05 (d, J=6.8 Hz, 3H), 0.92-0.82 (m, 3H).

Exemplary Synthesis of (2S,4R)-1-[(2S)-2-{3-[(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-7-azaspiro[3.5]nonan-2-yl)oxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 159)

Step 1: Preparation of (2S,4R)-1-[(2S)-2-[3-(7-azaspiro[3.5]nonan-2-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide

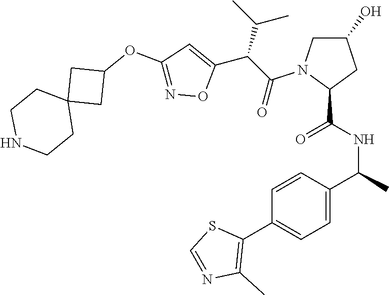 [SMILES: Cc1ncsc1-c1ccc([C@H](C)NC(=O)[C@@H]2C[C@@H](O)CN2C(=O)[C@H](c2cc(OC3CC4(CCNCC4)C3)no2)C(C)C)cc1]

To a solution of tert-butyl 2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy-7-azaspiro[3.5]nonane-7-carboxylate (391 mg, 0.5 mmol) in CH₂Cl₂ (5.0 mL) was added 4 M HCl in dioxane (5.0 mL). The mixture was stirred at 25° C. for 1 h. The mixture was concentrated under reduced pressure to give (2S,4R)-1-[(2S)-2-[3-(7-azaspiro[3.5]nonan-2-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide (501 mg, crude, hydrochloride salt) as a yellow solid, which was used directly in the next step. MS (ESI) m/z: 622.1 [M+H]⁺.

Step 2: Preparation of tert-butyl 3-[8-fluoro-7-[7-fluoro-3-hydroxy-8-(2-triisopropylsilylethynyl)-1-naphthyl]-2-[2-[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy-7-azaspiro[3.5]nonan-7-yl]ethoxy]pyrido[4,3-d]pyrimidin-4-yl]-3,8-diazabicyclo[3.2.1]octane-8-carboxylate

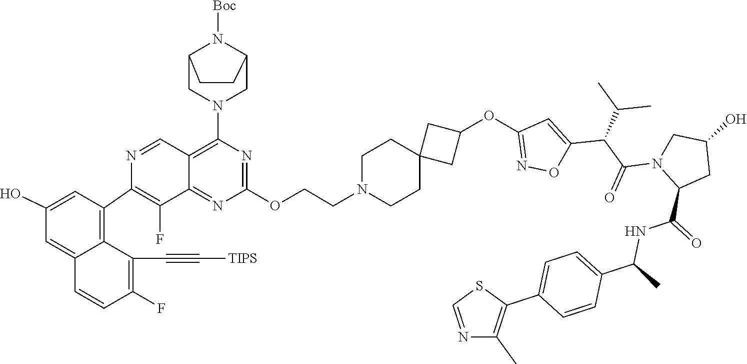

To a solution of (2S,4R)-1-[(2S)-2-[3-(7-azaspiro[3.5]nonan-2-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide hydrochloride (303 mg, 0.5 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (318 mg, 0.4 mmol) in CH₂Cl₂ (5.0 mL) and i-PrOH (5.0 mL) was added sodium acetate (344 mg, 4.2 mmol) and 2-methylpyridine borane (180 mg, 1.7 mmol). The mixture was stirred at 25° C. for 16 h. The reaction was filtered and concentrated under reduced pressure. The residue was purified by column chromatography (0-7% methanol (1N NH₃ as additive) in dichloromethane) to afford tert-butyl 3-[8-fluoro-7-[7-fluoro-3-hydroxy-8-(2-triisopropylsilylethynyl)-1-naphthyl]-2-[2-[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy-7-azaspiro[3.5]nonan-7-yl]ethoxy]pyrido[4,3-d]pyrimidin-4-yl]-3,8-diazabicyclo[3.2.1]octane-8-carboxylate (467 mg, 80%) as a yellow foam. MS (ESI) m/z: 1363.5 [M+H]⁺.

Step 3: Preparation of tert-butyl 3-[7-(8-ethynyl-7-fluoro-3-hydroxy-1-naphthyl)-8-fluoro-2-[2-[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy-7-azaspiro[3.5]nonan-7-yl]ethoxy]pyrido[4,3-d]pyrimidin-4-yl]-3,8-diazabicyclo[3.2.1]octane-8-carboxylate

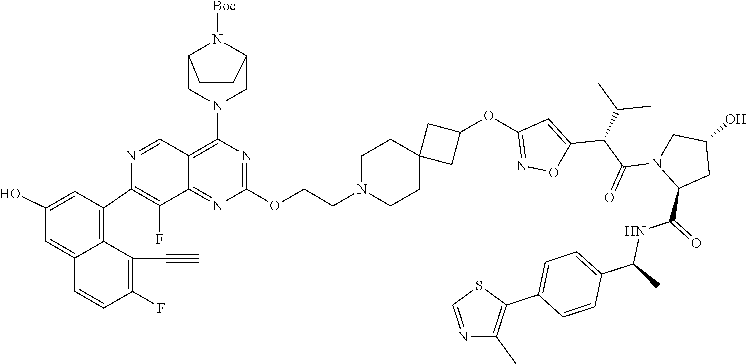 [SMILES: C#Cc1c(F)ccc2cc(O)cc(-c3ncc4c(N5CC6CCC(C5)N6C(=O)OC(C)(C)C)nc(OCCN5CCC6(CC5)CC(Oc5cc([C@@H](C(=O)N7C[C@H](O)C[C@H]7C(=O)N[C@@H](C)c7ccc(-c8scnc8C)cc7)C(C)C)on5)C6)nc4c3F)c12]

To a solution of tert-butyl 3-[8-fluoro-7-[7-fluoro-3-hydroxy-8-(2-triisopropylsilylethynyl)-1-naphthyl]-2-[2-[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy-7-azaspiro[3.5]nonan-7-yl]ethoxy]pyrido[4,3-d]pyrimidin-4-yl]-3,8-diazabicyclo[3.2.1]octane-8-carboxylate (467 mg, 0.3 mmol) in THF (5.0 mL) was added TBAF (0.67 mL, 1 M). The mixture was stirred at 25° C. for 12 h. The reaction mixture was diluted with ethyl acetate (40 mL) and washed with water (10 mL×10). The combined organic layers were washed brine (10 mL×2), dried over Na₂SO₄, filtered, and concentrated under reduced pressure to give tert-butyl 3-[7-(8-ethynyl-7-fluoro-3-hydroxy-1-naphthyl)-8-fluoro-2-[2-[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy-7-azaspiro[3.5]nonan-7-yl]ethoxy]pyrido[4,3-d]pyrimidin-4-yl]-3,8-diazabicyclo[3.2.1]octane-8-carboxylate (434 mg, crude) as a yellow solid, which was used directly in the next step. MS (ESI) m/z: 1207.5 [M+H]⁺.

Step 4: Preparation of (2S,4R)-1-[(2S)-2-[3-[[7-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-7-azaspiro[3.5]nonan-2-yl]oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

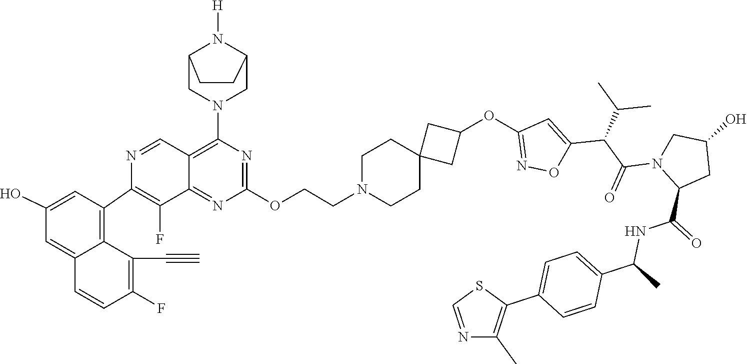 [SMILES: C#Cc1c(F)ccc2cc(O)cc(-c3ncc4c(N5CC6CCC(C5)N6)nc(OCCN5CCC6(CC5)CC(Oc5cc([C@@H](C(=O)N7C[C@H](O)C[C@H]7C(=O)N[C@@H](C)c7ccc(-c8scnc8C)cc7)C(C)C)on5)C6)nc4c3F)c12]

To a solution of tert-butyl 3-[7-(8-ethynyl-7-fluoro-3-hydroxy-1-naphthyl)-8-fluoro-2-[2-[2-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy-7-azaspiro[3.5]nonan-7-yl]ethoxy]pyrido[4,3-d]pyrimidin-4-yl]-3,8-diazabicyclo[3.2.1]octane-8-carboxylate (434 mg, 0.3 mmol) in CH₂Cl₂ (5 0 mL) was added 4 M HCl in dioxane (5.0 mL). The mixture was stirred at 25° C. for 30 min. The reaction mixture was diluted with methanol/dichloromethane (10/1 v/v, 110 mL). The organic layer was washed brine (20 mL), dried over Na₂SO₄, filtered, and concentrated under reduced pressure. The residue was purified by prep-HPLC (Column: Xtimate C18 150*40 mm*10 um; Eluent: 20%-60% acetonitrile in water (NH₃H₂O+NH₄HCO₃); Gradient time: 25 min; Hold time: 5 min; Flow rate: 25 mL/min) to afford (2S,4R)-1-[(2S)-2-[3-[7-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-7-azaspiro[3.5]nonan-2-yl]oxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (186.5 mg, 49%) as a yellow solid. MS (ESI) m/z: 1107.4 [M+H]⁺; ¹H NMR (400 MHz, CD₃OD) δ 8.99 (s, 1H), 8.88-8.85 (m, 1H), 7.84 (dd, J=5.6, 9.2 Hz, 1H), 7.46-7.39 (m, 2H), 7.36-7.33 (m, 3H), 7.32-7.27 (m, 1H), 7.20 (d, J=2.4 Hz, 1H), 5.98 (s, 1H), 4.99 (d, J=6.8 Hz, 1H), 4.61 (s, 4H), 4.58-4.53 (m, 3H), 4.42 (s, 1H), 3.78-3.71 (m, 2H), 3.69 (d, J=6.4 Hz, 2H), 3.66 (d, J=6.8 Hz, 3H), 3.37-3.35 (m, 1H), 2.83 (t, J=5.2 Hz, 2H), 2.66-2.48 (m, 4H), 2.48-2.46 (m, 3H), 2.37-2.33 (m, 2H), 2.25-2.17 (m, 1H), 1.95 (ddd, J=4.8, 8.4, 13.2 Hz, 1H), 1.88-1.77 (m, 6H), 1.69-1.62 (m, 4H), 1.47 (d, J=7.2 Hz, 3H), 1.04 (d, J=6.8 Hz, 3H), 0.95-0.88 (m, 3H).

Exemplary Synthesis of (2S,4R)-1-[(2S)-2-(3-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 160)

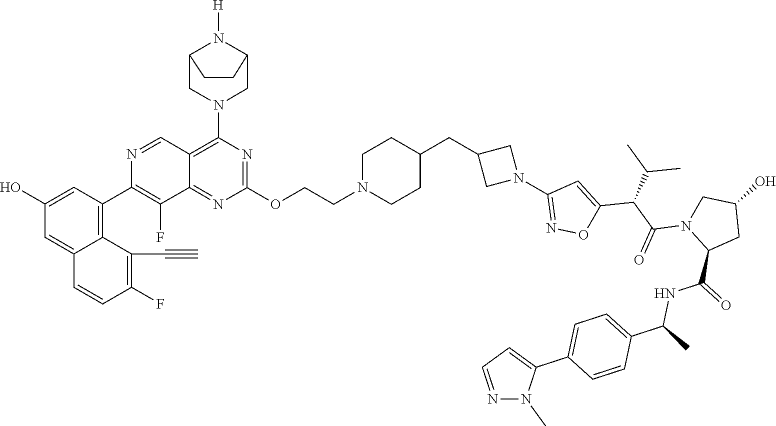 [SMILES: C#Cc1c(F)ccc2cc(O)cc(-c3ncc4c(N5CC6CCC(C5)N6)nc(OCCN5CCC(CC6CN(c7cc([C@@H](C(=O)N8C[C@H](O)C[C@H]8C(=O)N[C@@H](C)c8ccc(-c9ccnn9C)cc8)C(C)C)on7)C6)CC5)nc4c3F)c12]

The title compound was made analogously to (2S,4R)-1-[(2R)-2-(3-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide, starting from tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[[3-(4-piperidylmethyl) azetidin-1-yl]isoxazol-5-yl]butanoyl]-N-[(1S)-1-[4-(2-methylpyrazol-3-yl)phenyl]ethyl]pyrrolidine-2-carboxamide. (light brown solid). MS (ESI) m/z: 1103.6 [M+H]⁺; ¹H NMR (400 MHz, CD₃OD) δ 9.01 (s, 1H), 7.85 (dd, J=6.0, 9.2 Hz, 1H), 7.50-7.29 (m, 7H), 7.21 (d, J=2.4 Hz, 1H), 6.37-6.31 (m, 1H), 5.88-5.80 (m, 1H), 5.00 (q, J=6.8 Hz, 1H), 4.71-4.50 (m, 6H), 4.46-4.40 (m, 1H), 4.05-3.96 (m, 2H), 3.88-3.83 (m, 3H), 3.75-3.63 (m, 7H), 3.55-3.47 (m, 2H), 3.38 (s, 1H), 3.14-3.05 (m, 2H), 2.87 (t, J=5.6 Hz, 2H), 2.41-2.30 (m, 1H), 2.26-2.13 (m, 3H), 1.96 (ddd, J=4.8, 8.4, 13.2 Hz, 1H), 1.90-1.76 (m, 4H), 1.67 (d, J=8.4 Hz, 2H), 1.60-1.55 (m, 2H), 1.49 (d, J=7.2 Hz, 3H), 1.29 (s, 3H), 1.08-0.86 (m, 6H).

Exemplary Synthesis of (2S,4R)-1-[(2R)-2-{3-[(7-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-7-azaspiro[3.5]nonan-2-yl)oxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 161)

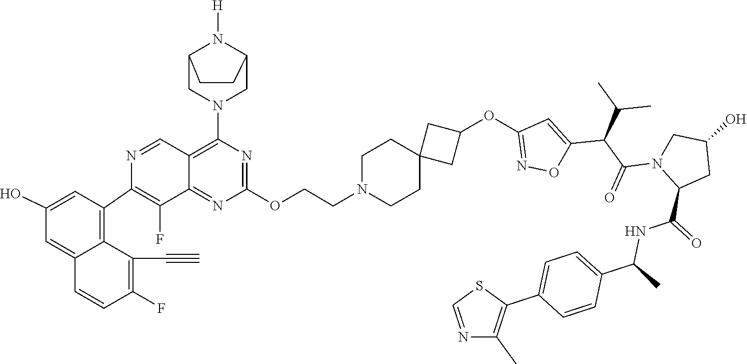 [SMILES: C#Cc1c(F)ccc2cc(O)cc(-c3ncc4c(N5CC6CCC(C5)N6)nc(OCCN5CCC6(CC5)CC(Oc5cc([C@H](C(=O)N7C[C@H](O)C[C@H]7C(=O)N[C@@H](C)c7ccc(-c8scnc8C)cc7)C(C)C)on5)C6)nc4c3F)c12]

The title compound was made analogously to (2S,4R)-1-[(2S)-2-(3-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)oxy]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide, starting from tert-butyl 2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxy-7-azaspiro[3.5]nonane-7-carboxylate and tert-butyl 3-[7-[3-tert-butoxycarbonyloxy-7-fluoro-8-(2-triisopropylsilylethynyl)-1-naphthyl]-8-fluoro-2-(2-oxoethoxy)pyrido[4,3-d]pyrimidin-4-yl]-3,8-diazabicyclo[3.2.1]octane-8-carboxylate. (yellow solid). MS (ESI) m/z: 1107.5 [M+H]⁺; ¹H NMR (400 MHz, CD₃OD) δ 9.02 (s, 1H), 8.89-8.87 (m, 1H), 7.87 (dd, J=5.6, 8.8 Hz, 1H), 7.47-7.39 (m, 4H), 7.37-7.30 (m, 2H), 7.22 (d, J=2.4 Hz, 1H), 5.98 (s, 1H), 5.04 (q, J=6.8 Hz, 1H), 4.63 (d, J=6.4 Hz, 4H), 4.57 (s, 1H), 4.51 (t, J=8.0 Hz, 1H), 4.44 (d, J=2.0 Hz, 1H), 3.84 (dd, J=4.0, 11.2 Hz, 1H), 3.76-3.71 (m, 2H), 3.70-3.65 (m, 3H), 3.65-3.58 (m, 1H), 3.38 (s, 1H), 2.87 (t, J=5.2 Hz, 2H), 2.69-2.51 (m, 4H), 2.50-2.48 (m, 3H), 2.44-2.38 (m, 2H), 2.23-2.15 (m, 1H), 2.00-1.84 (m, 6H), 1.84-1.79 (m, 2H), 1.71 (s, 4H), 1.60-1.51 (m, 3H), 1.06 (d, J=6.4 Hz, 3H), 0.89 (d, J=6.8 Hz, 3H).

Exemplary Synthesis of (2S,4R)-1-[(2R)-2-[3-(3-{[(3R)-3-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-4-methylpiperazin-1-yl]methyl}azetidin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 162)

Step 1: Preparation of tert-butyl(3R)-3-(hydroxymethyl)-4-methyl-piperazine-1-carboxylate

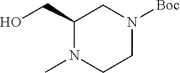 [SMILES: CN1CCN(C(=O)OC(C)(C)C)C[C@@H]1CO]

A solution of tert-butyl(3R)-3-(hydroxymethyl) piperazine-1-carboxylate (1.5 g, 7 mmol) and formaldehyde (382 uL, 14 mmol) in acetonitrile (21 mL) and water (4 mL) was stirred at 25° C. for 2 h, then NaBH(OAc)₃ (2.9 g, 14 mmol) was added. The mixture was stirred at 25° C. for 12 h. The pH of the mixture was adjusted to 8 with saturated sodium bicarbonate solution, diluted with water (20 mL) and extracted with CH₂Cl₂ (150 mL×3). The combined organic layers were washed with water (10 mL×5) and brine (15 mL×2), dried over anhydrous Na₂SO₄, filtered, and concentrated to afford tert-butyl(3R)-3-(hydroxymethyl)-4-methyl-piperazine-1-carboxylate (1.54 g, crude) as a yellow oil. MS (ESI) m/z: 231.1 [M+1]⁺.

Step 2: Preparation of [(2R)-1-methylpiperazin-2-yl]methanol

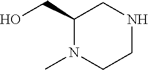

To a solution of tert-butyl(3R)-3-(hydroxymethyl)-4-methyl-piperazine-1-carboxylate (1.5 g, 6.5 mmol) in CH₂Cl₂ (6 mL) was added 4 M HCl in dioxane (3 mL). The mixture was stirred at 0° C. for 1 h. The reaction mixture was suspended in petroleum ether (30 mL), the precipitate was separated, dissolved in THF (30 mL) and adjusted pH to 8 with triethylamine. The mixture was filtered and concentrated under reduced pressure to afford [(2R)-1-methylpiperazin-2-yl]methanol (800 mg, crude) as a white solid, which was used directly in the next step.

Step 3: Preparation of 2-trimethylsilylethyl (3R)-3-(hydroxymethyl)-4-methyl-piperazine-1-carboxylate

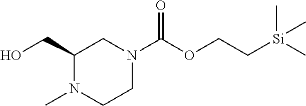 [SMILES: CN1CCN(C(=O)OCC[Si](C)(C)C)C[C@@H]1CO]

To a solution of [(2R)-1-methylpiperazin-2-yl]methanol (800 mg, 6.1 mmol) in H₂O (4 mL) and THF (20 mL) was added (2,5-dioxopyrrolidin-1-yl) 2-trimethylsilylethyl carbonate (1.8 g, 6.8 mmol) and NaHCO₃ (2.1 g, 24 mmol). The mixture was stirred at 20° C. for 15 h, then concentrated under reduced pressure. The residue was diluted with water (50 mL) and extracted with ethyl acetate (50 mL×3), the combined organic layer was dried over anhydrous Na₂SO₄, filtered and concentrated. The residue was purified by column chromatography (0-42% ethyl acetate in petroleum ether) to afford 2-trimethylsilylethyl (3R)-3-(hydroxymethyl)-4-methyl-piperazine-1-carboxylate (1.1 g, 62%) as a yellow liquid. MS (ESI) m/z: 275.17 [M+1]⁺.

Step 4: Preparation of tert-butyl 3-[7-[8-ethyl-3-(methoxymethoxy)-1-naphthyl]-8-fluoro-2-[[(2R)-1-methyl-4-(2-trimethylsilylethoxycarbonyl) piperazin-2-yl]methoxy]pyrido[4,3-d]pyrimidin-4-yl]-3,8-diazabicyclo[3.2.1]octane-8-carboxylate

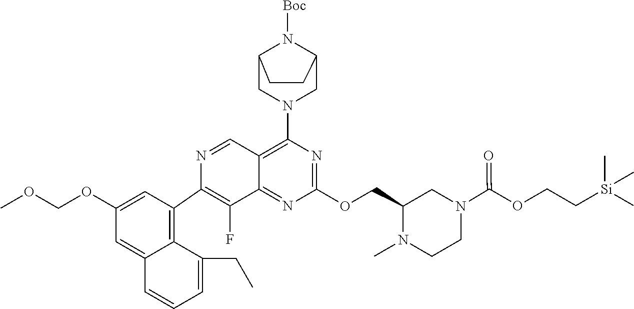

To a solution of 2-trimethylsilylethyl (3R)-3-(hydroxymethyl)-4-methyl-piperazine-1-carboxylate (602 mg, 2.2 mmol) and tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (1.1 g, 1.7 mmol) in toluene (4.0 mL) were added t-BuONa (324 mg, 3.4 mmol) and 4 A molecular sieves (147 mg). The mixture was stirred at 0° C. for 1 h, then quenched with aq. NH₄Cl until pH 6 and diluted with water (20 mL). The aqueous phase was extracted with ethyl acetate (40 mL×3). The combined organic phase was washed with brine (30 mL), dried over anhydrous Na₂SO₄, filtered and concentrated. The residue was purified by column chromatography (0-45% ethyl acetate in petroleum ether) to afford tert-butyl 3-[7-[8-ethyl-3-(methoxymethoxy)-1-naphthyl]-8-fluoro-2-[[(2R)-1-methyl-4-(2-trimethylsilylethoxycarbonyl) piperazin-2-yl]methoxy]pyrido[4,3-d]pyrimidin-4-yl]-3,8-diazabicyclo[3.2.1]octane-8-carboxylate (950 mg, 61%) as a yellow solid. MS (ESI) m/z: 846.0 [M+1]⁺.

Step 5: Preparation of tert-butyl 3-[7-[8-ethyl-3-(methoxymethoxy)-1-naphthyl]-8-fluoro-2-[[(2R)-1-methylpiperazin-2-yl]methoxy]pyrido[4,3-d]pyrimidin-4-yl]-3,8-diazabicyclo[3.2.1]octane-8-carboxylate

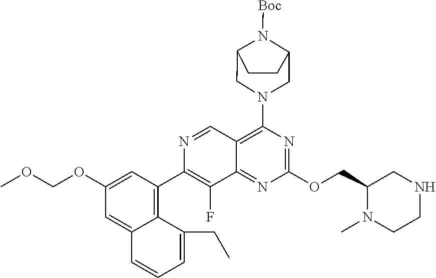 [SMILES: CCc1cccc2cc(OCOC)cc(-c3ncc4c(N5CC6CCC(C5)N6C(=O)OC(C)(C)C)nc(OC[C@H]5CNCCN5C)nc4c3F)c12]

To a solution of tert-butyl 3-[7-[8-ethyl-3-(methoxymethoxy)-1-naphthyl]-8-fluoro-2-[[(2R)-1-methyl-4-(2-trimethylsilylethoxycarbonyl) piperazin-2-yl]methoxy]pyrido[4,3-d]pyrimidin-4-yl]-3,8-diazabicyclo[3.2.1]octane-8-carboxylate (950 mg, 1.1 mmol) in DMF (8 mL) was added CsF (853 mg, 5.6 mmol). The mixture was stirred at 90° C. for 1 h, filtered, diluted with water (50 mL) and extracted with EtOAc (50 mL×3). The combined organic layer was dried over anhydrous Na₂SO₄, filtered, and concentrated under reduced pressure to afford tert-butyl 3-[7-[8-ethyl-3-(methoxymethoxy)-1-naphthyl]-8-fluoro-2-[[(2R)-1-methylpiperazin-2-yl]methoxy]pyrido[4,3-d]pyrimidin-4-yl]-3,8-diazabicyclo[3.2.1]octane-8-carboxylate (750 mg, crude) as a yellow solid, which was used directly in the next step. MS (ESI) m/z: 702.4 [M+1]⁺.

Step 6: Preparation of tert-butyl 3-[7-[8-ethyl-3-(methoxymethoxy)-1-naphthyl]-8-fluoro-2-[[(2R)-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-methyl-piperazin-2-yl]methoxy]pyrido[4,3-d]pyrimidin-4-yl]-3,8-diazabicyclo[3.2.1]octane-8-carboxylate

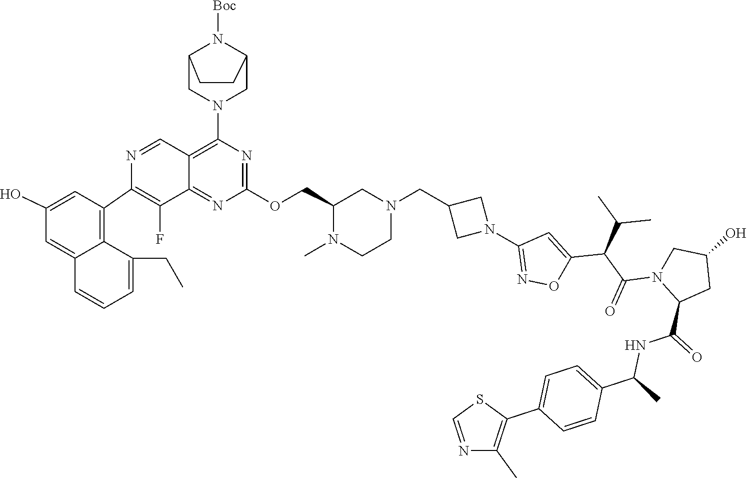 [SMILES: CCc1cccc2cc(O)cc(-c3ncc4c(N5CC6CCC(C5)N6C(=O)OC(C)(C)C)nc(OC[C@H]5CN(CC6CN(c7cc([C@H](C(=O)N8C[C@H](O)C[C@H]8C(=O)N[C@@H](C)c8ccc(-c9scnc9C)cc8)C(C)C)on7)C6)CCN5C)nc4c3F)c12]

To a solution of tert-butyl 3-[7-[8-ethyl-3-(methoxymethoxy)-1-naphthyl]-8-fluoro-2-[[(2R)-1-methylpiperazin-2-yl]methoxy]pyrido[4,3-d]pyrimidin-4-yl]-3,8-diazabicyclo[3.2.1]octane-8-carboxylate (400 mg, 0.6 mmol) and (2S,4R)-1-[(2R)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (316 mg, 0.5 mmol) in i-PrOH (1.0 mL) and CH₂Cl₂ (1.0 mL) were added acetic acid (109 uL, 1.9 mmol) and 2-methylpyridine borane (203 mg, 1.9 mmol). The mixture was stirred at 25° C. for 1 h, then concentrated in vacuum. The pH of the residue was adjusted to 10 by trimethylamine, purified by column chromatography (0-3% then up to 4% methanol (1N NH₃ as additive) in dichloromethane) to afford tert-butyl 3-[7-[8-ethyl-3-(methoxymethoxy)-1-naphthyl]-8-fluoro-2-[[(2R)-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-methyl-piperazin-2-yl]methoxy]pyrido[4,3-d]pyrimidin-4-yl]-3,8-diazabicyclo[3.2.1]octane-8-carboxylate (370 mg, 56%) as a white solid. MS (ESI) m/z: 1251.6 [M+1]⁺.

Step 7: Preparation of (2S,4R)-1-[(2R)-2-[3-[[3-[[(3R)-3-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-4-methyl-piperazin-1-yl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

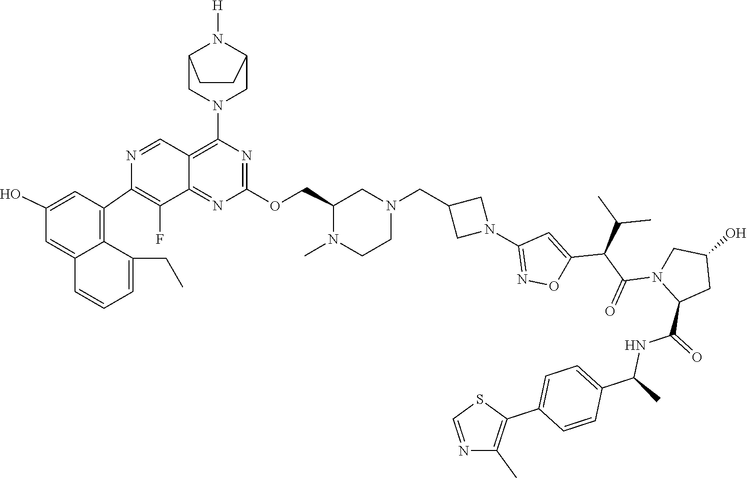 [SMILES: CCc1cccc2cc(O)cc(-c3ncc4c(N5CC6CCC(C5)N6)nc(OC[C@H]5CN(CC6CN(c7cc([C@H](C(=O)N8C[C@H](O)C[C@H]8C(=O)N[C@@H](C)c8ccc(-c9scnc9C)cc8)C(C)C)on7)C6)CCN5C)nc4c3F)c12]

To a solution of tert-butyl 3-[7-[8-ethyl-3-(methoxymethoxy)-1-naphthyl]-8-fluoro-2-[[(2R)-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-methyl-piperazin-2-yl]methoxy]pyrido[4,3-d]pyrimidin-4-yl]-3,8-diazabicyclo[3.2.1]octane-8-carboxylate (370 mg, 0.3 mmol) in CH₂Cl₂ (3.0 mL) was added TFA (1.5 mL). The mixture was stirred at 25° C. for 0.5 h, added saturated aqueous NaHCO₃ solution to adjust the pH to 8. The resulting mixture was extracted with dichloromethane/methanol (120 mL, 10:1, V/V). The combined organic phase was washed with brine (30 mL), dried with anhydrous Na₂SO₄, filtered and concentrated. The residue was purified by preparative HPLC (column: Xtimate C18 150*40 mm*10 um; mobile phase: [water (NH₃H₂O+NH₄HCO₃)-ACN]; gradient: 18%-58% B over 25 min) to afford (2S,4R)-1-[(2R)-2-[3-[[3-[[(3R)-3-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-4-methyl-piperazin-1-yl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (94.7 mg, 28%) as a white solid. MS (ESI) m/z: 1108.7 [M+1]⁺; 1H NMR (400 MHz, CD₃OD) δ 9.10 (s, 1H), 8.88 (s, 1H), 8.44 (s, 1H), 7.64 (d, J=7.6 Hz, 1H), 7.47-7.33 (m, 5H), 7.32-7.27 (m, 1H), 7.17 (d, J=6.8 Hz, 1H), 7.03 (d, J=4.0 Hz, 1H), 5.84 (d, J=4.0 Hz, 1H), 5.06-4.99 (m, 1H), 4.68-4.56 (m, 6H), 4.51-4.46 (m, 1H), 4.45 (s, 1H), 4.02 (m, 2H), 3.69 (m, 9H), 3.00 (m, 2H), 2.85 (d, J=12.0 Hz, 1H), 2.76 (d, J=8.0 Hz, 1H), 2.67-2.62 (m, 3H), 2.50-2.45 (m, 6H), 2.40-2.20 (m, 6H), 2.01-1.94 (m, 1H), 1.89-1.79 (m, 3H), 1.60-1.47 (m, 3H), 1.05 (d, J=4.0 Hz, 3H), 0.95-0.87 (m, 6H).

Exemplary Synthesis of (2S,4R)-1-[(2R)-2-[3-(3-{[(3S)-3-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-4-methylpiperazin-1-yl]methyl}azetidin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 163)

Step 1: Preparation of tert-butyl(3S)-3-(hydroxymethyl)-4-methyl-piperazine-1-carboxylate

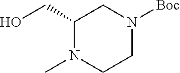 [SMILES: CN1CCN(C(=O)OC(C)(C)C)C[C@H]1CO]

A solution of tert-butyl(3S)-3-(hydroxymethyl) piperazine-1-carboxylate (1.0 g, 4.6 mmol) and formaldehyde (255 uL, 9 mmol) in acetonitrile (21 mL) and water (4 mL) was stirred at 25° C. for 2 h, then NaBH(OAc)₃ (1.96 g, 9 mmol) was added. The mixture was stirred at 25° C. for 2 h, saturated aqueous NaHCO₃ was added to adjust the pH to 8. The reaction mixture was diluted with H₂O (20 mL) and extracted with CH₂Cl₂ (150 mL×3). The combined organic layers were washed with water (10 mL×5), brine (15 mL×2), dried over anhydrous Na₂SO₄, filtered and concentrated to afford tert-butyl(3S)-3-(hydroxymethyl)-4-methyl-piperazine-1-carboxylate (1.05 g, crude) as a yellow oil, which was used directly in the next step. MS (ESI) m/z: 231.1 [M+1]⁺

Step 2: Preparation of [(2S)-1-methylpiperazin-2-yl]methanol

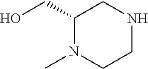

To a solution of tert-butyl(3S)-3-(hydroxymethyl)-4-methyl-piperazine-1-carboxylate (1.0 g, 4.3 mmol) in CH₂Cl₂ (6 mL) was added HCl in dioxane (4 M, 5.4 mL). The mixture was stirred at 0° C. for 1 h. The reaction was suspended in petroleum ether (30 mL), filtered and the solid was dissolved with THF (30 mL), adjusted the pH to 8 with triethylamine. The mixture was filtered and concentrated under reduced pressure to afford [(2S)-1-methylpiperazin-2-yl]methanol (580 mg, crude) as a white solid, which was used directly in the next step.

Step 3: Preparation of 2-trimethylsilylethyl (3S)-3-(hydroxymethyl)-4-methyl-piperazine-1-carboxylate

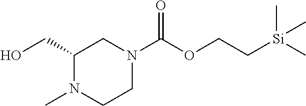 [SMILES: CN1CCN(C(=O)OCC[Si](C)(C)C)C[C@H]1CO]

To a solution of [(2S)-1-methylpiperazin-2-yl]methanol (580 mg, 4.5 mmol) in H₂O (4 mL) and THF (20 mL) was added (2,5-dioxopyrrolidin-1-yl) 2-trimethylsilylethyl carbonate (1.3 g, 4.9 mmol) and NaHCO₃ (1.5 g, 18 mmol). The mixture was stirred at 20° C. for 15 h, then concentrated under reduced pressure. The residue was diluted with water (50 mL) and extracted with EtOAc (50 mL×3), the combined organic layer was dried over anhydrous Na₂SO₄, filtered and concentrated under reduced pressure. The residue was purified by column chromatography (0-40% ethyl acetate in petroleum ether) to afford 2-trimethylsilylethyl (3S)-3-(hydroxymethyl)-4-methyl-piperazine-1-carboxylate (700 mg, 54%) as a yellow liquid. MS (ESI) m/z: 275.17 [M+1]⁺.

Step 4: Preparation of tert-butyl 3-[7-[8-ethyl-3-(methoxymethoxy)-1-naphthyl]-8-fluoro-2-[[(2S)-1-methyl-4-(2-trimethylsilylethoxycarbonyl) piperazin-2-yl]methoxy]pyrido[4,3-d]pyrimidin-4-yl]-3,8-diazabicyclo[3.2.1]octane-8-carboxylate

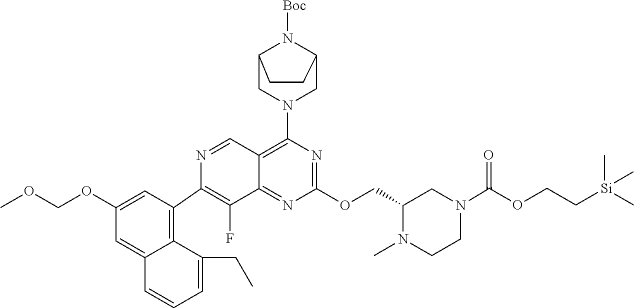

To a solution of 2-trimethylsilylethyl (3S)-3-(hydroxymethyl)-4-methyl-piperazine-1-carboxylate (700 mg, 2.4 mmol) and tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (1.4 g, 2.1 mmol) in toluene (4.0 mL) was added t-BuONa (409 mg, 4.2 mmol). The mixture was stirred at 0° C. for 1 h, saturated ammonium chloride solution was added to the reaction to adjust the PH to 7. The reaction mixture was diluted with ethyl acetate (200 mL) and filtered. The filtrate washed with water (60 mL×6), brine (60 mL), dried over anhydrous sodium sulfate, filtered, and concentrated. The residue was purified by column chromatography (0-45% ethyl acetate in petroleum ether) to afford tert-butyl 3-[7-[8-ethyl-3-(methoxymethoxy)-1-naphthyl]-8-fluoro-2-[[(2S)-1-methyl-4-(2-trimethylsilylethoxycarbonyl) piperazin-2-yl]methoxy]pyrido[4,3-d]pyrimidin-4-yl]-3,8-diazabicyclo[3.2.1]octane-8-carboxylate (1.07 g, 56%) as a yellow solid. MS (ESI) m/z: 846.5 [M+1]⁺.

Step 5: Preparation of tert-butyl 3-[7-[8-ethyl-3-(methoxymethoxy)-1-naphthyl]-8-fluoro-2-[[(2S)-1-methylpiperazin-2-yl]methoxy]pyrido[4,3-d]pyrimidin-4-yl]-3,8-diazabicyclo[3.2.1]octane-8-carboxylate

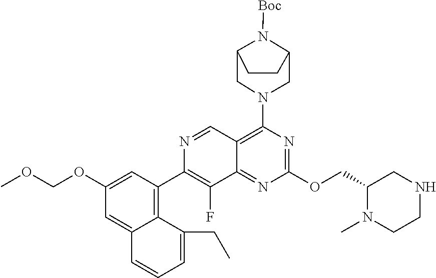 [SMILES: CCc1cccc2cc(OCOC)cc(-c3ncc4c(N5CC6CCC(C5)N6C(=O)OC(C)(C)C)nc(OC[C@@H]5CNCCN5C)nc4c3F)c12]

To a solution of tert-butyl 3-[7-[8-ethyl-3-(methoxymethoxy)-1-naphthyl]-8-fluoro-2-[[(2S)-1-methyl-4-(2-trimethylsilylethoxycarbonyl) piperazin-2-yl]methoxy]pyrido[4,3-d]pyrimidin-4-yl]-3,8-diazabicyclo[3.2.1]octane-8-carboxylate (1.07 g, 1.2 mmol) in DMF (8 mL) was added CsF (958 mg, 6.3 mmol). The reaction was stirred at 90° C. for 1 h. The mixture was filtered and diluted with water (50 mL), extracted with EtOAc (50 mL×3), the combined organic layer was dried over anhydrous Na₂SO₄, filtered, and concentrated under reduced pressure to afford tert-butyl 3-[7-[8-ethyl-3-(methoxymethoxy)-1-naphthyl]-8-fluoro-2-[[(2S)-1-methylpiperazin-2-yl]methoxy]pyrido[4,3-d]pyrimidin-4-yl]-3,8-diazabicyclo[3.2.1]octane-8-carboxylate (789 mg, 89%) as a yellow solid.

Step 6: Preparation of tert-butyl 3-[7-[8-ethyl-3-(methoxymethoxy)-1-naphthyl]-8-fluoro-2-[[(2S)-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-methyl-piperazin-2-yl]methoxy]pyrido[4,3-d]pyrimidin-4-yl]-3,8-diazabicyclo[3.2.1]octane-8-carboxylate

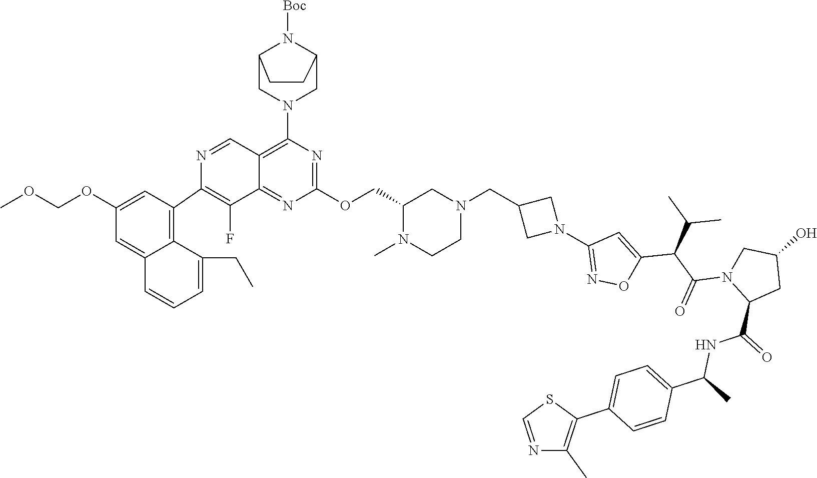 [SMILES: CCc1cccc2cc(OCOC)cc(-c3ncc4c(N5CC6CCC(C5)N6C(=O)OC(C)(C)C)nc(OC[C@@H]5CN(CC6CN(c7cc([C@H](C(=O)N8C[C@H](O)C[C@H]8C(=O)N[C@@H](C)c8ccc(-c9scnc9C)cc8)C(C)C)on7)C6)CCN5C)nc4c3F)c12]

To a solution of tert-butyl 3-[7-[8-ethyl-3-(methoxymethoxy)-1-naphthyl]-8-fluoro-2-[[(2S)-1-methylpiperazin-2-yl]methoxy]pyrido[4,3-d]pyrimidin-4-yl]-3,8-diazabicyclo[3.2.1]octane-8-carboxylate (138 mg, 0.2 mmol) and (2S,4R)-1-[(2R)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (157 mg, 0.2 mmol) in i-PrOH (1 mL) and CH₂Cl₂ (1 mL) were added acetic acid (45 uL, 0.8 mmol) and 2-methylpyridine borane (84 mg, 0.8 mmol). The mixture was stirred at 25° C. for 2 h, then concentrated in vacuum. The pH of the residue was adjusted to 10 by trimethylamine and purified by column chromatography (0-3% then up to 4% methanol (1N NH₃ as additive) in dichloromethane) to afford tert-butyl 3-[7-[8-ethyl-3-(methoxymethoxy)-1-naphthyl]-8-fluoro-2-[[(2S)-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-methyl-piperazin-2-yl]methoxy]pyrido[4,3-d]pyrimidin-4-yl]-3,8-diazabicyclo[3.2.1]octane-8-carboxylate (160 mg, 41%) as a white solid. MS (ESI) m/z: 1251.5 [M+1]⁺.

Step 7: Preparation of (2S,4R)-1-[(2R)-2-[3-[[3-[[(3S)-3-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-4-methyl-piperazin-1-yl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

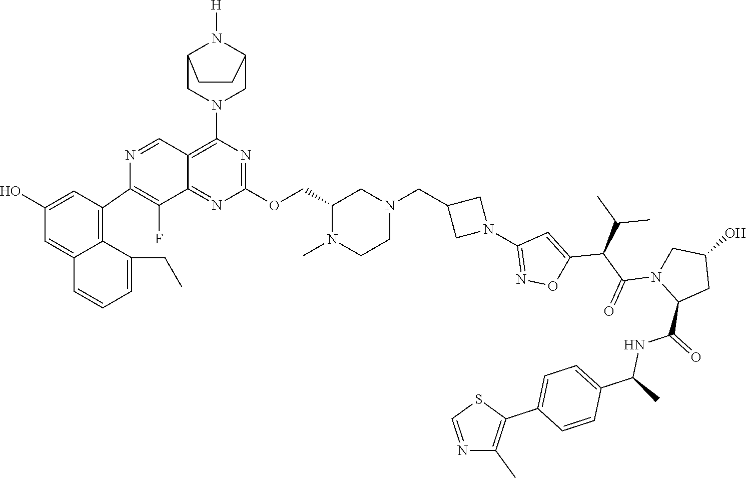 [SMILES: CCc1cccc2cc(O)cc(-c3ncc4c(N5CC6CCC(C5)N6)nc(OC[C@@H]5CN(CC6CN(c7cc([C@H](C(=O)N8C[C@H](O)C[C@H]8C(=O)N[C@@H](C)c8ccc(-c9scnc9C)cc8)C(C)C)on7)C6)CCN5C)nc4c3F)c12]

To a solution of tert-butyl 3-[7-[8-ethyl-3-(methoxymethoxy)-1-naphthyl]-8-fluoro-2-[[(2S)-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-methyl-piperazin-2-yl]methoxy]pyrido[4,3-d]pyrimidin-4-yl]-3,8-diazabicyclo[3.2.1]octane-8-carboxylate (160 mg, 0.1 mmol) in CH₂Cl₂ (3 mL) was added TFA (1.5 mL). The mixture was stirred at 25° C. for 0.5 h, saturated aqueous NaHCO₃ was added to adjust the pH to 8. The resulting mixture was extracted with dichloromethane/methanol (120 mL, 10:1, V/V). The combined organic phase was washed with brine (30 mL), dried with anhydrous Na₂SO₄, filtered and concentrated. The residue was purified by preparative HPLC (column: Welch Xtimate C18 150*30 mm*5 um; mobile phase: [water (NH₃H₂O+NH₄HCO₃)-acetonitrile]; gradient: 24%-64% B over 28 min) to afford (2S,4R)-1-[(2R)-2-[3-[[3-[[(3S)-3-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-4-methyl-piperazin-1-yl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (81.4 mg, 23%) as a white solid. MS (ESI) m/z: 1107.6 [M+1]⁺; 1H NMR (400 MHz, CD₃OD) δ 9.10 (d, J=6.0 Hz, 1H), 8.94-8.83 (m, 1H), 7.64 (d, J=8.2 Hz, 1H), 7.52-7.27 (m, 6H), 7.20-7.10 (m, 1H), 7.07-6.97 (m, 1H), 5.82 (d, J=14.0 Hz, 1H), 5.05 (s, 1H), 4.63-4.60 (m, 6H), 4.56-4.51 (m, 1H), 4.49-4.42 (m, 1H), 4.11-3.97 (m, 2H), 3.87-3.80 (m, 1H), 3.78-3.57 (m, 8H), 3.04 (d, J=8.2 Hz, 2H), 2.93-2.84 (m, 1H), 2.78-2.66 (m, 3H), 2.50-2.18 (m, 12H), 2.01-1.68 (m, 5H), 1.65-1.47 (m, 3H), 1.05 (d, J=6.0 Hz, 3H), 0.95-0.81 (m, 6H).

Exemplary Synthesis of (2S,4R)-1-[(2S)-2-[3-(3-{[(3S)-3-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-4-methylpiperazin-1-yl]methyl}azetidin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 164)

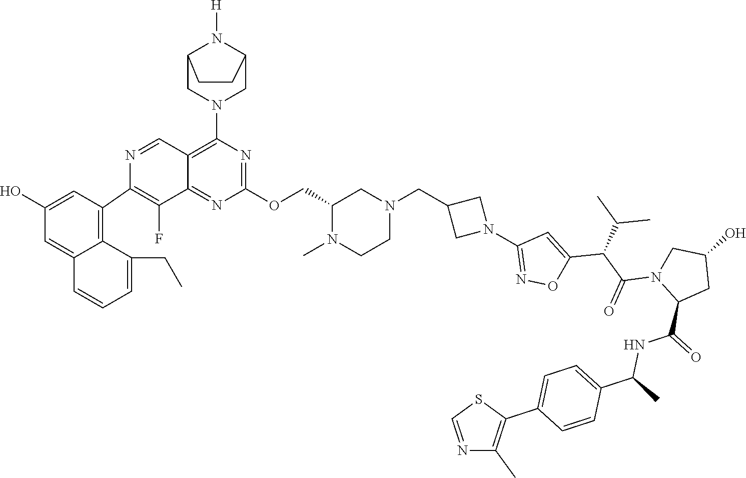 [SMILES: CCc1cccc2cc(O)cc(-c3ncc4c(N5CC6CCC(C5)N6)nc(OC[C@@H]5CN(CC6CN(c7cc([C@@H](C(=O)N8C[C@H](O)C[C@H]8C(=O)N[C@@H](C)c8ccc(-c9scnc9C)cc8)C(C)C)on7)C6)CCN5C)nc4c3F)c12]

The title compound was made in an analogous manner to (2S,4R)-1-[(2R)-2-[3-(3-{[(3S)-3-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-4-methylpiperazin-1-yl]methyl}azetidin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide starting from tert-butyl 3-[7-[8-ethyl-3-(methoxymethoxy)-1-naphthyl]-8-fluoro-2-[[(2S)-1-methylpiperazin-2-yl]methoxy]pyrido[4,3-d]pyrimidin-4-yl]-3,8-diazabicyclo[3.2.1]octane-8-carboxylate and (2S,4R)-1-[(2S)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). MS (ESI) m/z: 1107.7 [M+1]⁺; 1H NMR (400 MHz, CD₃OD) δ 9.06 (s, 1H), 8.88 (s, 1H), 8.44 (s, 1H), 7.63 (d, J=7.6 Hz, 1H), 7.47-7.33 (m, 5H), 7.32-7.27 (m, 1H), 7.17 (d, J=6.8 Hz, 1H), 7.03 (d, J=4.0 Hz, 1H), 5.88 (d, J=4.0 Hz, 1H), 5.06-4.99 (m, 1H), 4.68-4.56 (m, 6H), 4.51-4.46 (m, 1H), 4.45 (s, 1H), 4.02 (m, 2H), 3.69 (m, 9H), 3.00 (m, 2H), 2.85 (d, J=12.0 Hz, 1H), 2.76 (d, J=8.0 Hz, 1H), 2.67-2.62 (m, 3H), 2.50-2.45 (m, 6H), 2.40-2.20 (m, 6H), 2.01-1.94 (m, 1H), 1.89-1.79 (m, 3H), 1.60-1.47 (m, 3H), 1.05 (d, J=4.0 Hz, 3H), 0.95-0.86 (m, 6H).

Exemplary Synthesis of (2S,4R)-1-[(2R)-2-{3-[[3-({1-[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) azetidin-3-yl]piperidin-4-yl}methyl) azetidin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 165)

Step 1: Preparation of tert-butyl-3-[7-[8-ethyl-3-(methoxymethoxy)-1-naphthyl]-8-fluoro-2-(3-hydroxyazetidin-1-yl)pyrido[4,3-d]pyrimidin-4-yl]-3,8-diazabicyclo[3.2.1]octane-8-carboxylate

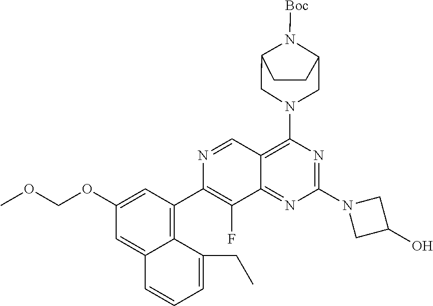 [SMILES: CCc1cccc2cc(OCOC)cc(-c3ncc4c(N5CC6CCC(C5)N6C(=O)OC(C)(C)C)nc(N5CC(O)C5)nc4c3F)c12]

To a solution of azetidin-3-ol; hydrochloride (403 mg, 3.7 mmol) in THF (10 mL) was added DIEA (1.3 mL, 8 mmol) and stirred at 45° C. for 20 min, then tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (1.0 g, 1.5 mmol) was added at 25° C. The mixture was stirred at 25° C. for 16 h, then concentrated under reduced pressure. The residue was purified by column chromatography (0-40% ethyl acetate in petroleum ether) to afford tert-butyl-3-[7-[8-ethyl-3-(methoxymethoxy)-1-naphthyl]-8-fluoro-2-(3-hydroxyazetidin-1-yl)pyrido[4,3-d]pyrimidin-4-yl]-3,8-diazabicyclo[3.2.1]octane-8-carboxylate (976 mg, 96%) as a white solid. MS (ESI) m/z: 645.3 [M+1]⁺.

Step 2: Preparation of tert-butyl-3-[7-[8-ethyl-3-(methoxymethoxy)-1-naphthyl]-8-fluoro-2-(3-oxoazetidin-1-yl)pyrido[4,3-d]pyrimidin-4-yl]-3,8-diazabicyclo[3.2.1]octane-8-carboxylate

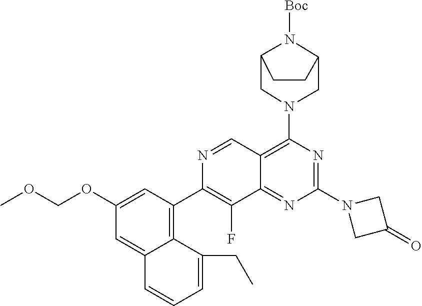 [SMILES: CCc1cccc2cc(OCOC)cc(-c3ncc4c(N5CC6CCC(C5)N6C(=O)OC(C)(C)C)nc(N5CC(=O)C5)nc4c3F)c12]

To a solution of tert-butyl 3-[7-[8-ethyl-3-(methoxymethoxy)-1-naphthyl]-8-fluoro-2-(3-hydroxyazetidin-1-yl)pyrido[4,3-d]pyrimidin-4-yl]-3,8-diazabicyclo[3.2.1]octane-8-carboxylate (976 mg, 1.5 mmol) in CH₂Cl₂ (20 mL) were added PhI(OAc)₂ (731 mg, 2.3 mmol) and TEMPO (238 mg, 1.5 mmol). The mixture was stirred at 25° C. for 16 h. The mixture was diluted with dichloromethane (80 mL), washed with brine (10 mL×2), dried over anhydrous sodium sulfate, filtered, and concentrated in vacuum. The residue was purified by column chromatography (10-33% ethyl acetate in petroleum ether) to afford tert-butyl-3-[7-[8-ethyl-3-(methoxymethoxy)-1-naphthyl]-8-fluoro-2-(3-oxoazetidin-1-yl)pyrido[4,3-d]pyrimidin-4-yl]-3,8-diazabicyclo[3.2.1]octane-8-carboxylate (857 mg, 88%) as a yellow solid. MS (ESI) m/z: 643.7 [M+1]⁺.

Step 3: Preparation of tert-butyl-3-[7-[8-ethyl-3-(methoxymethoxy)-1-naphthyl]-8-fluoro-2-[3-[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]azetidin-1-yl]pyrido[4,3-d]pyrimidin-4-yl]-3,8-diazabicyclo[3.2.1]octane-8-carboxylate

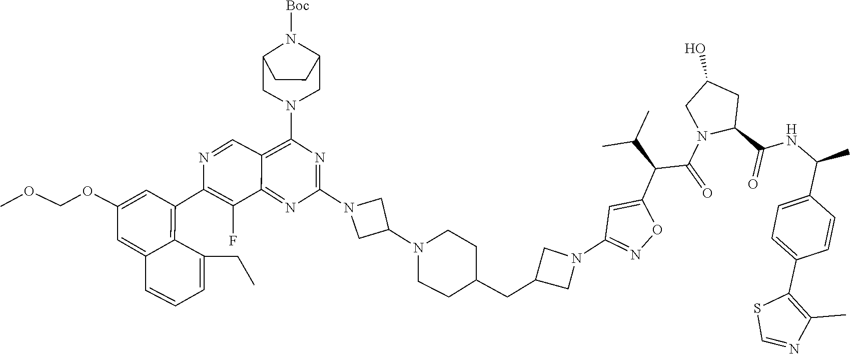 [SMILES: CCc1cccc2cc(OCOC)cc(-c3ncc4c(N5CC6CCC(C5)N6C(=O)OC(C)(C)C)nc(N5CC(N6CCC(CC7CN(c8cc([C@H](C(=O)N9C[C@H](O)C[C@H]9C(=O)N[C@@H](C)c9ccc(-c%10scnc%10C)cc9)C(C)C)on8)C7)CC6)C5)nc4c3F)c12]

To a solution of tert-butyl 3-[7-[8-ethyl-3-(methoxymethoxy)-1-naphthyl]-8-fluoro-2-(3-oxoazetidin-1-yl)pyrido[4,3-d]pyrimidin-4-yl]-3,8-diazabicyclo[3.2.1]octane-8-carboxylate and (2S,4R)-4-hydroxy-1-[(2R)-3-methyl-2-[3-[[3-(4-(410 mg, 0.6 mmol) piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide (405 mg, 0.6 mmol) in CH₂Cl₂ (5 mL) and i-PrOH (5 mL) were added HOAc (153 mg, 2.6 mmol) and 2-methylpyridine borane (341 mg, 3.2 mmol). The mixture was stirred at 25° C. for 16 h, then concentrated under reduced pressure. The residue was purified by column chromatography (0-8% then up to 10% methanol in dichloromethane) to afford tert-butyl-3-[7-[8-ethyl-3-(methoxymethoxy)-1-naphthyl]-8-fluoro-2-[3-[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]azetidin-1-yl]pyrido[4,3-d]pyrimidin-4-yl]-3,8-diazabicyclo[3.2.1]octane-8-carboxylate (620 mg, 76%) as a yellow solid. MS (ESI) m/z: 631.7 [M/2+1]⁺.

Step 4: Preparation of (2S,4R)-1-[(2R)-2-[3-[[3-[[1-[1-[4-(3,8-diazabicyclo[3.2.1]octan-3-yl)-7-(8-ethyl-3-hydroxy-1-naphthyl)-8-fluoro-pyrido[4,3-d]pyrimidin-2-yl]azetidin-3-yl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

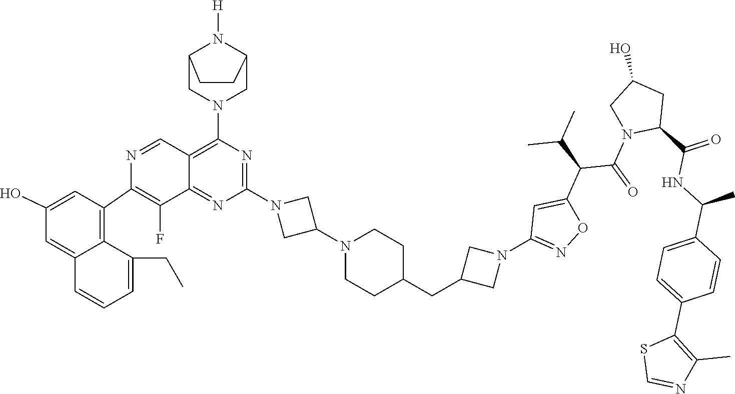 [SMILES: CCc1cccc2cc(O)cc(-c3ncc4c(N5CC6CCC(C5)N6)nc(N5CC(N6CCC(CC7CN(c8cc([C@H](C(=O)N9C[C@H](O)C[C@H]9C(=O)N[C@@H](C)c9ccc(-c%10scnc%10C)cc9)C(C)C)on8)C7)CC6)C5)nc4c3F)c12]

To a solution of tert-butyl 3-[7-[8-ethyl-3-(methoxymethoxy)-1-naphthyl]-8-fluoro-2-[3-[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]azetidin-1-yl]pyrido[4,3-d]pyrimidin-4-yl]-3,8-diazabicyclo[3.2.1]octane-8-carboxylate (400 mg, 0.3 mmol) in CH₂Cl₂ (10 mL) was added TsOH·H₂O (603 mg, 3 mmol). The mixture was stirred at 25° C. for 2 h, quenched by sat. NaHCO₃ (10 mL), extracted with dichloromethane/methanol (30 mL×3, 10/1). The combined organic phase was washed with brine (20 mL), dried over anhydrous sodium sulfate, filtered, and concentrated in vacuum. The residue was purified by prep-HPLC (column: Xtimate C18 150*40 mm*10 um; mobile phase: [water (formic acid)-acetonitrile]; B %: 0%-36%, 36 min) to afford (2S,4R)-1-[(2R)-2-[3-[[3-[[1-[1-[4-(3,8-diazabicyclo[3.2.1]octan-3-yl)-7-(8-ethyl-3-hydroxy-1-naphthyl)-8-fluoro-pyrido[4,3-d]pyrimidin-2-yl]azetidin-3-yl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (96.8 mg, formic acid salt, 26%) as a white solid. MS (ESI) m/z: 1117.7 [M+1]⁺; 1H NMR (400 MHz, CD₃OD) δ 8.88 (s, 1H), 8.81 (s, 1H), 8.52-8.39 (m, 1H), 7.62 (d, J=8.0 Hz, 1H), 7.49-7.32 (m, 5H), 7.28 (d, J=2.4 Hz, 1H), 7.16 (d, J=7.2 Hz, 1H), 6.99 (d, J=2.4 Hz, 1H), 5.91-5.75 (m, 1H), 5.09-4.97 (m, 1H), 4.72-4.56 (m, 3H), 4.51 (t, J=8.4 Hz, 1H), 4.47-4.38 (m, 1H), 4.33 (t, J=8.4 Hz, 2H), 4.19-3.97 (m, 6H), 3.88-3.68 (m, 3H), 3.66-3.45 (m, 4H), 3.03-2.83 (m, 3H), 2.48 (s, 3H), 2.42-2.26 (m, 3H), 2.23-1.91 (m, 8H), 1.81-1.70 (m, 2H), 1.64 (t, J=6.4 Hz, 2H), 1.59-1.48 (m, 3H), 1.46-1.21 (m, 3H), 1.04 (d, J=6.4 Hz, 3H), 0.96-0.83 (m, 6H).

Exemplary Synthesis of (2S,4R)-1-[(2R)-2-{3-[4-({4-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]piperidin-1-yl}methyl) piperidin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 166)

Step 1: Preparation of 2-trimethylsilylethyl 4-hydroxypiperidine-1-carboxylate

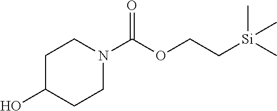 [SMILES: C[Si](C)(C)CCOC(=O)N1CCC(O)CC1]

To a solution of piperidin-4-ol (500 mg, 4.94 mmol, 1.0 eq) in CH₂Cl₂ (10 mL) was added Et₃N (2.1 mL, 14.83 mmol) stirred at 25° C. Then the (2,5-dioxopyrrolidin-1-yl) 2-trimethylsilylethyl carbonate (1.9 g, 7.41 mmol, 1.5 eq) was added and stirred at 20° C. for 16 hours. TLC (acidic silica gel, petroleum ether:ethyl acetate=1:1, Rf=0.50) indicated one major spot formed. The reaction mixture was diluted with water (20 mL) and extracted with CH₂Cl₂ (30 mL×3). The combined organic phase was washed with brine (20 mL), dried with anhydrous Na₂SO₄, filtered and concentrated. The residue was purified by column chromatography (0-25% ethyl acetate in petroleum ether) to afford 2-trimethylsilylethyl 4-hydroxypiperidine-1-carboxylate (1.3 g, crude) as a white oil. ¹H NMR (400 MHz, CDCl₃) δ 4.21-4.16 (m, 2H), 3.87 (dt, J=4.2, 8.4 Hz, 2H), 3.14-3.07 (m, 2H), 1.89-1.86 (m, 1H), 1.59 (s, 2H), 1.52 (d, J=4.0 Hz, 2H), 1.03-0.98 (m, 2H), 0.91-0.77 (m, 2H), 0.05 (s, 8H).

Step 2: Preparation of tert-butyl 3-[7-[8-ethyl-3-(methoxymethoxy)-1-naphthyl]-8-fluoro-2-[[1-(2-trimethylsilylethoxycarbonyl)-4-piperidyl]oxy]pyrido[4,3-d]pyrimidin-4-yl]-3,8-diazabicyclo[3.2.1]octane-8-carboxylate

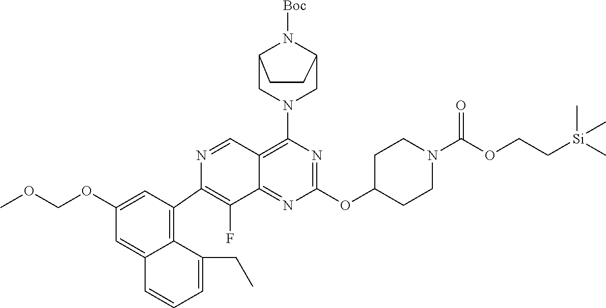

To a solution of 2-trimethylsilylethyl 4-hydroxypiperidine-1-carboxylate (249 mg, 1 mmol) and tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (550 mg, 0.8 mmol) in toluene (10.0 mL) was added 4 A molecular sieves (1.0 g). Then t-BuONa (203 mg, 2 mmol) was added at 0° C. The mixture was stirred at 0° C. for 1 h, aqueous NH₄Cl was added to adjust the pH to 7 and diluted with water (20 mL). The aqueous phase was extracted with ethyl acetate (30 mL×3). The combined organic phase was washed with brine (20 mL), dried over anhydrous Na₂SO₄, filtered and concentrated. The residue was purified column chromatography (0-28% ethyl acetate in petroleum ether) to afford tert-butyl 3-[7-[8-ethyl-3-(methoxymethoxy)-1-naphthyl]-8-fluoro-2-[[1-(2-trimethylsilylethoxycarbonyl)-4-piperidyl]oxy]pyrido[4,3-d]pyrimidin-4-yl]-3,8-diazabicyclo[3.2.1]octane-8-carboxylate (581 mg, 78%) as a white foam. MS (ESI) m/z: 817.8 [M+1]⁺.

Step 3: Preparation of tert-butyl 3-[7-[8-ethyl-3-(methoxymethoxy)-1-naphthyl]-8-fluoro-2-(4-piperidyloxy)pyrido[4,3-d]pyrimidin-4-yl]-3,8-diazabicyclo[3.2.1]octane-8-carboxylate

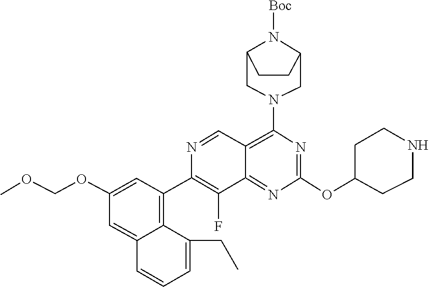 [SMILES: CCc1cccc2cc(OCOC)cc(-c3ncc4c(N5CC6CCC(C5)N6C(=O)OC(C)(C)C)nc(OC5CCNCC5)nc4c3F)c12]

To a solution of tert-butyl 3-[7-[8-ethyl-3-(methoxymethoxy)-1-naphthyl]-8-fluoro-2-[[1-(2-trimethylsilylethoxycarbonyl)-4-piperidyl]oxy]pyrido[4,3-d]pyrimidin-4-yl]-3,8-diazabicyclo[3.2.1]octane-8-carboxylate (581 mg, 0.7 mmol) in DMF (6 mL) was added CsF (539 mg, 3.6 mmol). The mixture was stirred at 90° C. for 1 h, then diluted with water (30 mL) and extracted with ethyl acetate (30 mL×3). The combined organic phase was washed with water (15 mL×5), brine (15 mL), dried over anhydrous sodium sulfate, filtered and concentrated in vacuum to give tert-butyl 3-[7-[8-ethyl-3-(methoxymethoxy)-1-naphthyl]-8-fluoro-2-(4-piperidyloxy)pyrido[4,3-d]pyrimidin-4-yl]-3,8-diazabicyclo[3.2.1]octane-8-carboxylate (440 mg, 92%) as a yellow solid. MS (ESI) m/z: 673.2 [M+1]⁺.

Step 4: Preparation of benzyl 4-(dimethoxymethyl)piperidine-1-carboxylate

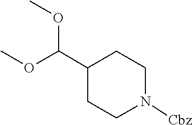 [SMILES: COC(OC)C1CCN(C(=O)OCc2ccccc2)CC1]

To a solution of benzyl 4-formylpiperidine-1-carboxylate (10.0 g, 40 mmol) in MeOH (80 mL) were added TsOH·H₂O (385 mg, 2 mmol) and trimethoxymethane (22.0 mL, 202 mmol). The mixture was stirred at 25° C. for 3 h. The reaction mixture was concentrated in vacuum, the residue was purified by column chromatography (0-14% ethyl acetate in petroleum ether) to afford benzyl 4-(dimethoxymethyl)piperidine-1-carboxylate (11.0 g, 93%) as a colorless oil. MS (ESI) m/z: 316.0 [M+23]⁺; ¹H NMR (400 MHz, CDCl₃) δ 7.41-7.28 (m, 5H), 5.13 (s, 2H), 4.31-4.12 (m, 2H), 4.03 (d, J=6.8 Hz, 1H), 3.35 (s, 6H), 2.75 (s, 2H), 1.80-1.71 (m, 3H), 1.30-1.18 (m, 2H)

Step 5: Preparation of 4-(dimethoxymethyl)piperidine

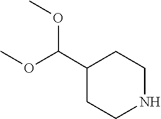

To a solution of benzyl 4-(dimethoxymethyl)piperidine-1-carboxylate (5.7 g, 19 mmol) in MeOH (60 mL) was added 10% Pd/C (700 mg) under N₂ atmosphere. The suspension was degassed and purged with H₂ for 3 times. The mixture was stirred under H₂ (15 Psi) at 25° C. for 16 h. The reaction mixture was filtered through Celite, concentrated in vacuum to give 4-(dimethoxymethyl)piperidine (3.0 g, 97%) as a white solid. ¹H NMR (400 MHz, CDCl₃) δ 4.43-4.33 (m, 1H), 4.03 (d, J=6.8 Hz, 1H), 3.33 (s, 6H), 3.23-3.09 (m, 2H), 2.62 (t, J=12.4 Hz, 2H), 1.81-1.69 (m, 3H), 1.38-1.24 (m, 2H).

Step 6: Preparation of methyl 2-[3-[4-(dimethoxymethyl)-1-piperidyl]isoxazol-5-yl]-3-methyl-butanoate

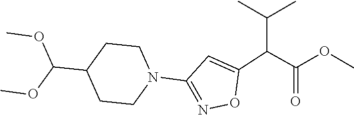

To a solution of 4-(dimethoxymethyl)piperidine (1.6 g, 10 mmol) and methyl 3-methyl-2-[3-(1,1,2,2,3,3,4,4,4-nonafluorobutylsulfonyloxy)isoxazol-5-yl]butanoate (4.0 g, 8 mmol) in DMA (90 mL) were added 4 A molecular sieves (6 g) and triethylamine (3.5 mL, 25 mmol). The mixture was stirred at 130° C. for 16 h, then cooled to room temperature and diluted with water (400 mL), filtered. The filtrate was extracted with ethyl acetate (200 mL×3). The combined organic phase was washed with water (50 mL×6), brine (50 mL×2), dried over anhydrous sodium sulfate, filtered, and concentrated over vacuum. The residue was purified by column chromatography (0-14% ethyl acetate in petroleum ether) to afford methyl 2-[3-[4-(dimethoxymethyl)-1-piperidyl]isoxazol-5-yl]-3-methyl-butanoate (1.4 g, 48%) as a yellow oil. MS (ESI) m/z: 341.1 [M+1]⁺; 1H NMR (400 MHz, CDCl₃) δ 5.91 (s, 1H), 4.06 (d, J=6.8 Hz, 1H), 3.75-3.69 (m, 5H), 3.47 (d, J=8.8 Hz, 1H), 3.37 (s, 6H), 2.85-2.76 (m, 2H), 2.38-2.28 (m, 1H), 1.84-1.74 (m, 3H), 1.44-1.32 (m, 2H), 1.00 (d, J=6.8 Hz, 3H), 0.92 (d, J=6.8 Hz, 3H).

Step 7: Preparation of 2-[3-[4-(dimethoxymethyl)-1-piperidyl]isoxazol-5-yl]-3-methyl-butanoic acid

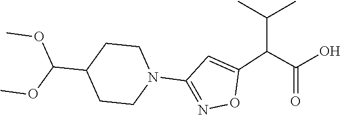 [SMILES: COC(OC)C1CCN(c2cc(C(C(=O)O)C(C)C)on2)CC1]

To a solution of methyl 2-[3-[4-(dimethoxymethyl)-1-piperidyl]isoxazol-5-yl]-3-methyl-butanoate (1.4 g, 4.1 mmol) in tetrahydrofuran (20 mL) and H₂O (10 mL) was added LiOH·H₂O (518 mg, 12 mmol). The mixture was stirred at 25° C. for 1 h, then aqueous HCl (2 M) was added to adjust the pH to 5-6, extracted with ethyl acetate (40 mL×3). The combined organic phase was washed with brine (30 mL), dried over anhydrous sodium sulfate, filtered, and concentrated in vacuum to give 2-[3-[4-(dimethoxymethyl)-1-piperidyl]isoxazol-5-yl]-3-methyl-butanoic acid (1.4 g, 98%) as a yellow oil. MS (ESI) m/z: 327.1 [M+1]⁺.

Step 8: Preparation of (2S,4R)-1-[2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

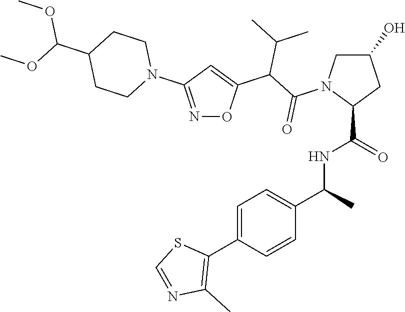 [SMILES: COC(OC)C1CCN(c2cc(C(C(=O)N3C[C@H](O)C[C@H]3C(=O)N[C@@H](C)c3ccc(-c4scnc4C)cc3)C(C)C)on2)CC1]

To a solution of 2-[3-[4-(dimethoxymethyl)-1-piperidyl]isoxazol-5-yl]-3-methyl-butanoic acid (1.3 g, 4 mmol) and (2S,4R)-4-hydroxy-N-[(1S)-1-[4-(4-methylthiazol-5-yl)phenyl]ethyl]pyrrolidine-2-carboxamide (1.9 g, 4 mmol) in DMF (30 mL) was added DIEA (3.5 mL, 20 mmol) and HATU (1.7 g, 4 mmol). The mixture was stirred at 25° C. for 1 h, then diluted with water (150 mL) and extracted with ethyl acetate (150 mL×3). The combined organic phase was washed with water (50 mL×5), brine (50 mL×2), dried over anhydrous sodium sulfate, filtered, and concentrated in vacuum. The residue was purified by column chromatography (0-6% methanol in dichloromethane) to afford (2S,4R)-1-[2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (2.3 g, 83%) as a yellow solid. MS (ESI) m/z: 640.4 [M+1]⁺.

Step 9: Preparation of (2S,4R)-1-[(2S)-2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide and (2S,4R)-1-[(2R)-2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

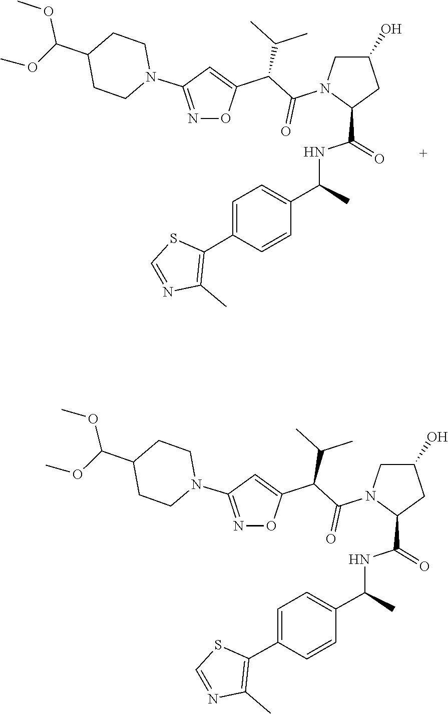 [SMILES: COC(OC)C1CCN(c2cc([C@@H](C(=O)N3C[C@H](O)C[C@H]3C(=O)N[C@@H](C)c3ccc(-c4scnc4C)cc3)C(C)C)on2)CC1 ; COC(OC)C1CCN(c2cc([C@H](C(=O)N3C[C@H](O)C[C@H]3C(=O)N[C@@H](C)c3ccc(-c4scnc4C)cc3)C(C)C)on2)CC1]

(2S,4R)-1-[2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (2.3 g) was separated by chiral SFC (column: DAICEL CHIRALPAK AD (250 mm*50 mm, 10 um); mobile phase: [0.1% NH₃H₂O IPA]; B %: 45%-45%). Fraction 1 was obtained as (2S,4R)-1-[(2S)-2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (940 mg, 41%) as a white solid. MS (ESI) m/z: 662.2 [M+23]⁺. Fraction 2 was obtained as (2S,4R)-1-[(2R)-2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (840 mg, 36%) as a white solid. MS (ESI) m/z: 662.2 [M+23]⁺.

Step 10: Preparation of (2S,4R)-1-[(2R)-2-[3-(4-formyl-1-piperidyl)isoxazol-5-yl]-3-methyl-butanoyl]-4-hydroxy-N-[(1S)-1-[4-(4-methylthiazol-5-yl)phenyl]ethyl]pyrrolidine-2-carboxamide

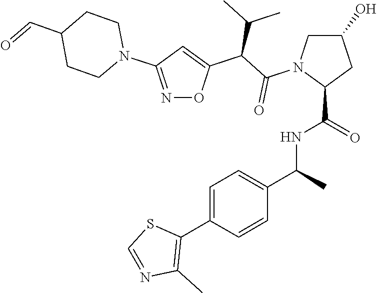 [SMILES: Cc1ncsc1-c1ccc([C@H](C)NC(=O)[C@@H]2C[C@@H](O)CN2C(=O)[C@@H](c2cc(N3CCC(C=O)CC3)no2)C(C)C)cc1]

To a solution of (2S,4R)-1-[(2R)-2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (400 mg, 0.6 mmol) in tetrahydrofuran (4.0 mL) was added HCl (4.0 mL, 2 M). The mixture was stirred at 25° C. for 30 h, then saturated aqueous NaHCO₃ was added to adjust the pH to 8 and extracted with ethyl acetate (20 mL×3). The combined organic phase was washed with brine (10 mL), dried over anhydrous sodium sulfate, filtered and concentrated in vacuum to give (2S,4R)-1-[(2R)-2-[3-(4-formyl-1-piperidyl)isoxazol-5-yl]-3-methyl-butanoyl]-4-hydroxy-N-[(1S)-1-[4-(4-methylthiazol-5-yl)phenyl]ethyl]pyrrolidine-2-carboxamide (380 mg, 97%) as a yellow solid. MS (ESI) m/z: 594.2 [M+1]⁺.

Step 11: Preparation of tert-butyl 3-[7-[8-ethyl-3-(methoxymethoxy)-1-naphthyl]-8-fluoro-2-[[1-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]-4-piperidyl]oxy]pyrido[4,3-d]pyrimidin-4-yl]-3,8-diazabicyclo[3.2.1]octane-8-carboxylate

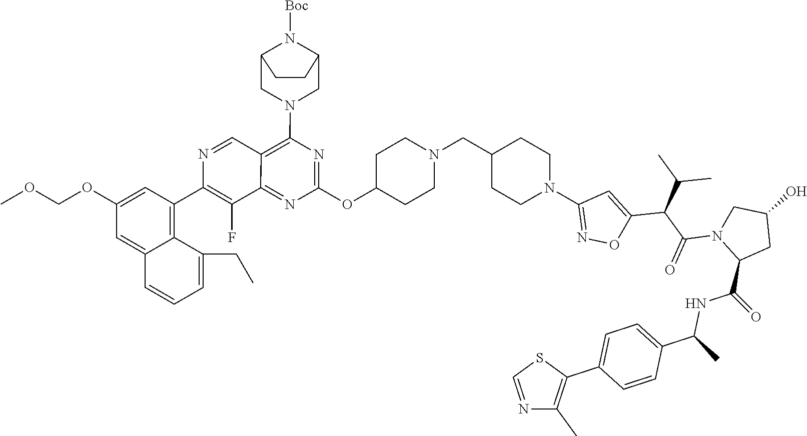 [SMILES: CCc1cccc2cc(OCOC)cc(-c3ncc4c(N5CC6CCC(C5)N6C(=O)OC(C)(C)C)nc(OC5CCN(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a solution of (2S,4R)-1-[(2R)-2-[3-(4-formyl-1-piperidyl)isoxazol-5-yl]-3-methyl-butanoyl]-4-hydroxy-N-[(1S)-1-[4-(4-methylthiazol-5-yl)phenyl]ethyl]pyrrolidine-2-carboxamide (233 mg, 0.4 mmol) and tert-butyl 3-[7-[8-ethyl-3-(methoxymethoxy)-1-naphthyl]-8-fluoro-2-(4-piperidyloxy)pyrido[4,3-d]pyrimidin-4-yl]-3,8-diazabicyclo[3.2.1]octane-8-carboxylate (220 mg, 0.3 mmol) in CH₂Cl₂ (4.0 mL) and i-PrOH (2.0 mL) was added 2-methylpyridine borane (175 mg, 1.6 mmol) and acetic acid (94 uL, 1.63 mmol). The mixture was stirred at 25° C. for 1 h, then concentrated. The residue was purified by column chromatography (0-7% methanol in dichloromethane) to afford tert-butyl 3-[7-[8-ethyl-3-(methoxymethoxy)-1-naphthyl]-8-fluoro-2-[[1-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]-4-piperidyl]oxy]pyrido[4,3-d]pyrimidin-4-yl]-3,8-diazabicyclo[3.2.1]octane-8-carboxylate (385 mg, 80%) as a yellow solid. MS (ESI) m/z: 626.1 [M/2+1]⁺.

Step 12: Preparation of (2S,4R)-1-[(2R)-2-[3-[4-[[4-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxy-1-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

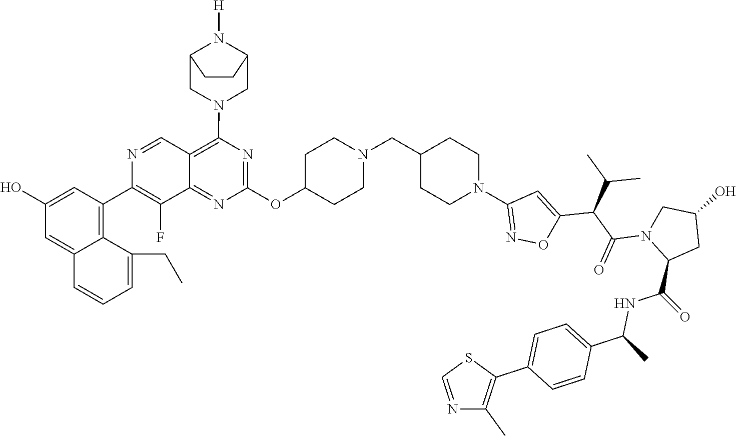 [SMILES: CCc1cccc2cc(O)cc(-c3ncc4c(N5CC6CCC(C5)N6)nc(OC5CCN(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a solution of tert-butyl 3-[7-[8-ethyl-3-(methoxymethoxy)-1-naphthyl]-8-fluoro-2-[[1-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]-4-piperidyl]oxy]pyrido[4,3-d]pyrimidin-4-yl]-3,8-diazabicyclo[3.2.1]octane-8-carboxylate (385 mg, 0.3 mmol) in CH₂Cl₂ (4.0 mL) was added 4 M HCl in dioxane (4.1 mL). The mixture was stirred at 25° C. for 30 min, suspended in petroleum ether (50 mL) and filtered. The filter cake was dissolved with tetrahydrofuran (30 mL), diluted with triethylamine (3 mL), filtered and concentrated. The residue was purified by prep-HPLC (column: Xtimate C18 150*40 mm*10 um; mobile phase: [water (FA)-ACN]; B %: 0%-36%, 36 min) to afford (2S,4R)-1-[(2R)-2-[3-[4-[[4-[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxy-1-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (123.3 mg, formic acid salt, 34%) as a white solid. MS (ESI) m/z: 1106.6 [M+1]⁺; 1H NMR (400 MHz, CD₃OD) δ 9.07 (s, 1H), 8.87 (s, 1H), 7.64 (d, J=8.0 Hz, 1H), 7.47-7.34 (m, 5H), 7.30 (d, J=2.4 Hz, 1H), 7.17 (d, J=6.8 Hz, 1H), 7.01 (d, J=2.4 Hz, 1H), 6.14-6.03 (m, 1H), 5.38 (s, 1H), 5.04 (d, J=6.8 Hz, 1H), 4.75-4.64 (m, 2H), 4.51 (t, J=8.0 Hz, 1H), 4.44 (s, 1H), 3.95-3.92 (m, 2H), 3.87-3.69 (m, 5H), 3.66-3.55 (m, 2H), 3.22-3.11 (m, 2H), 3.03-2.84 (m, 4H), 2.78-2.66 (m, 2H), 2.48 (s, 3H), 2.41-2.31 (m, 2H), 2.29-3.15 (m, 3H), 2.15-2.06 (m, 2H), 2.04-1.91 (m, 6H), 1.85 (d, J=12.0 Hz, 2H), 1.60-1.49 (m, 3H), 1.35-1.31 (m, 2H), 1.05 (d, J=6.4 Hz, 3H), 0.94-0.87 (m, 6H).

Exemplary Synthesis of (2S,4R)-1-[(2S)-2-{3-[4-({4-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]piperidin-1-yl}methyl) piperidin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 167)

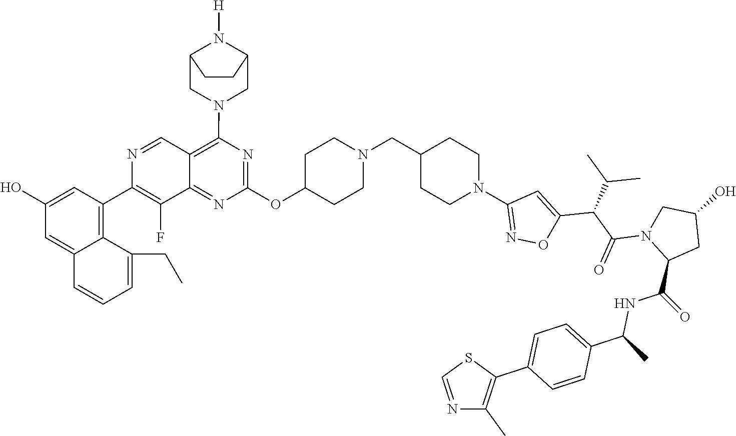 [SMILES: CCc1cccc2cc(O)cc(-c3ncc4c(N5CC6CCC(C5)N6)nc(OC5CCN(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-{3-[4-({4-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]piperidin-1-yl}methyl) piperidin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide, starting from (2S,4R)-1-[(2S)-2-[3-[4-(dimethoxymethyl)-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-[8-ethyl-3-(methoxymethoxy)-1-naphthyl]-8-fluoro-2-(4-piperidyloxy)pyrido[4,3-d]pyrimidin-4-yl]-3,8-diazabicyclo[3.2.1]octane-8-carboxylate. (formic acid salt, white solid). MS (ESI) m/z: 1106.6 [M+1]⁺; ¹H NMR (400 MHz, CD₃OD) δ 9.07 (s, 1H), 8.87 (s, 1H), 7.63 (d, J=8.4 Hz, 1H), 7.48-7.33 (m, 5H), 7.30 (d, J=2.4 Hz, 1H), 7.19-7.14 (m, 1H), 7.01 (d, J=2.4 Hz, 1H), 6.13 (s, 1H), 5.39 (s, 1H), 4.99 (d, J=7.2 Hz, 1H), 4.75-4.65 (m, 2H), 4.58 (t, J=8.0 Hz, 1H), 4.43 (s, 1H), 3.96-3.94 (m, 2H), 3.86-3.78 (m, 2H), 3.75-3.61 (m, 5H), 3.20-3.16 (m, 2H), 3.03-2.92 (m, 2H), 2.87-2.82 (m, 2H), 2.73 (d, J=6.4 Hz, 2H), 2.47 (s, 3H), 2.40-2.32 (m, 1H), 2.30-2.20 (m, 4H), 2.17-2.08 (m, 2H), 1.99-1.93 (m, 6H), 1.85-1.79 (m, 2H), 1.50 (d, J=7.2 Hz, 3H), 1.37-1.25 (m, 2H), 1.06 (d, J=6.4 Hz, 3H), 0.95-0.87 (m, 6H).

Exemplary Synthesis of (2S,4R)-1-[(2R)-2-(3-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]piperidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 168)

Step 1: Preparation of tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate

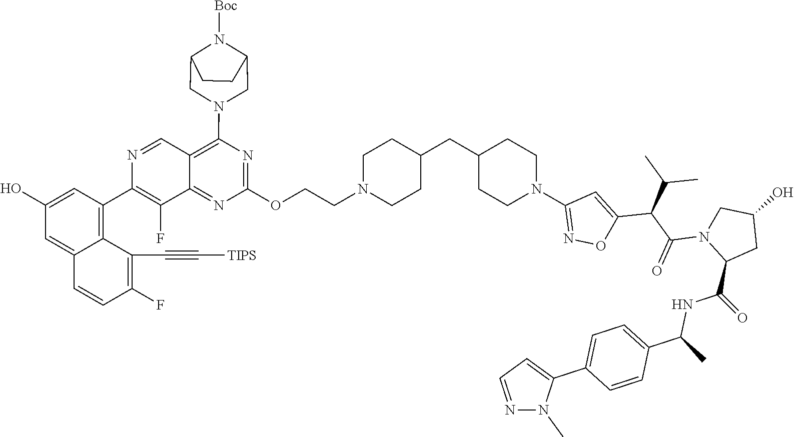

To a solution of tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (284 mg, 0.4 mmol) and (2S,4R)-4-hydroxy-1-[(2R)-3-methyl-2-[3-[4-(4-piperidylmethyl)-1-piperidyl]isoxazol-5-yl]butanoyl]-N-[(1S)-1-[4-(2-methylpyrazol-3-yl)phenyl]ethyl]pyrrolidine-2-carboxamide (220 mg, 0.3 mmol) in CH₂Cl₂ (4 mL) and i-PrOH (4 mL) were added acetic acid (0.08 mL, 1.4 mmol) and 2-picoline borane (146 mg, 1.4 mmol). The mixture was stirred at 25° C. for 1 h, then basified with triethylamine until pH 7 and concentrated under reduced pressure. The residue was purified by column chromatography (0-7% methanol in dichloromethane) to afford tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (458 mg, 93%) as light yellow oil. MS (ESI) m/z: 1387.7 [M+H]⁺.

Step 2: Preparation of tert-butyl 3-[7-(8-ethynyl-7-fluoro-3-hydroxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate

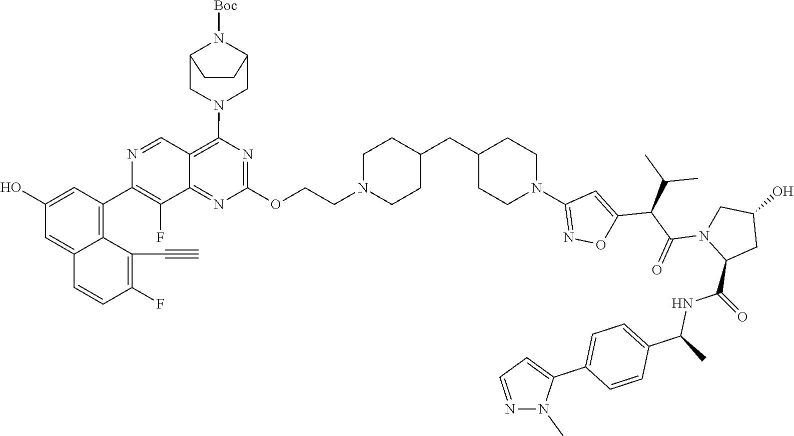 [SMILES: C#Cc1c(F)ccc2cc(O)cc(-c3ncc4c(N5CC6CCC(C5)N6C(=O)OC(C)(C)C)nc(OCCN5CCC(CC6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12]

To a solution of tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (458 mg, 0.32 mmol) in THF (6 mL) was added TBAF (1 M, 1.27 mL). The mixture was stirred at 25° C. for 16 h, then diluted with ethyl acetate (50 mL), and then washed with water (10 mL×5). The combined organic phase was dried over anhydrous sodium sulfate, filtered and concentrated under reduced pressure to afford tert-butyl 3-[7-(8-ethynyl-7-fluoro-3-hydroxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (321 mg, crude) as a light yellow solid, which was used directly in the next step. MS (ESI) m/z: 1231.6 [M+H]⁺.

Step 3: Preparation of (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

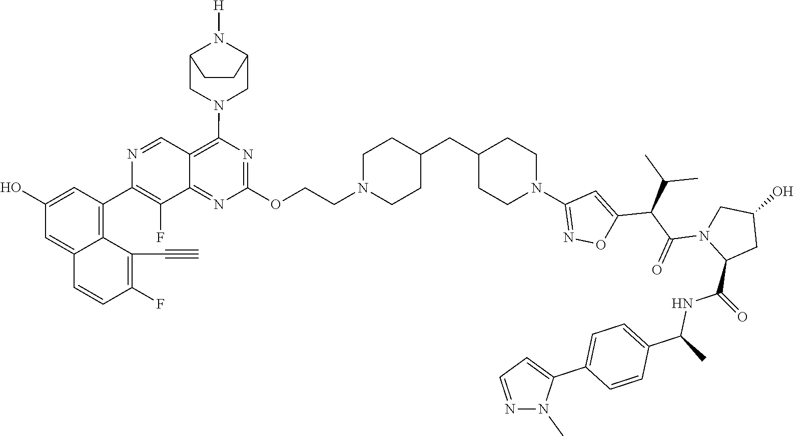 [SMILES: C#Cc1c(F)ccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12]

To a stirred solution of tert-butyl 3-[7-(8-ethynyl-7-fluoro-3-hydroxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (321 mg, 0.25 mmol) in formic acid (10 mL) was stirred at 25° C. for 1.5 h. The reaction mixture was concentrated under reduced pressure at 30° C., quenched by saturated NaHCO₃ solution (20 mL) and stirred for 30 min. The mixture was extracted with dichloromethane/methanol (20 mL×3, v/v=10/1). The combined organic layers were dried over Na₂SO₄, filtered, and concentrated under reduced pressure. The crude product was purified by prep-HPLC (column: Phenomenex C18 75*30 mm*3 um; mobile phase: [water (NH₄HCO₃)-MeCN]; B %: 30%-70%, Gradient Time: 25 min, Flow Rate: 25 mL/min) to afford (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (81.5 mg, 29%) as a light yellow solid. MS (ESI) m/z: 1131.7 [M+H]⁺; ¹H NMR (400 MHz, CD₃OD) δ 9.01 (s, 1H), 7.88-7.84 (m, 1H), 7.49-7.48 (m, 1H), 7.45-7.41 (m, 4H), 7.36-7.30 (m, 2H), 7.21-7.20 (m, 1H), 6.35-6.30 (m, 1H), 6.07 (s, 1H), 5.05-5.01 (m, 1H), 4.65-4.57 (m, 7H), 4.44 (s, 1H), 3.86 (s, 3H), 3.74-3.59 (m, 8H), 3.37 (s, 1H), 3.13-3.08 (m, 2H), 2.90-2.79 (m, 4H), 2.38-2.15 (m, 4H), 1.96-1.60 (m, 10H), 1.53-1.51 (m, 3H), 1.28-1.18 (m, 6H), 1.05-1.04 (m, 3H), 0.89-0.87 (m, 3H).

Exemplary Synthesis of (2S,4R)-1-[(2S)-2-(3-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]piperidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 169)

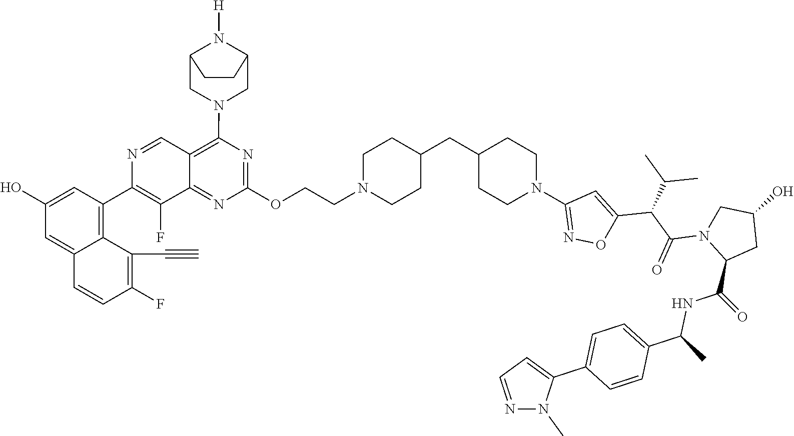 [SMILES: C#Cc1c(F)ccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12]

The title compound was made in an analogous manner to (2S,4R)-1-[(2R)-2-(3-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]piperidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide starting from tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[4-(4-piperidylmethyl)-1-piperidyl]isoxazol-5-yl]butanoyl]-N-[(1S)-1-[4-(2-methylpyrazol-3-yl)phenyl]ethyl]pyrrolidine-2-carboxamide. (light yellow solid). MS (ESI) m/z: 1131.7 [M+H]⁺; ¹H NMR (400 MHz, CD₃OD) δ 9.02 (s, 1H), 7.88-7.84 (m, 1H), 7.48-7.30 (m, 7H), 7.21-7.20 (m, 1H), 6.33-6.32 (m, 1H), 6.11 (s, 1H), 5.01-4.99 (m, 1H), 4.67-4.57 (m, 7H), 4.43 (s, 1H), 3.84 (s, 3H), 3.73-3.60 (m, 8H), 3.38 (s, 1H), 3.16-3.13 (m, 2H), 2.95-2.92 (m, 2H), 2.80-2.73 (m, 2H), 2.40-2.20 (m, 4H), 1.99-1.60 (m, 10H), 1.50-1.48 (m, 3H), 1.32-1.25 (m, 2H), 1.19-1.15 (m, 4H), 1.06-1.04 (m, 3H), 0.92-0.90 (m, 3H).

Exemplary Synthesis of (2S,4R)-1-[(2S)-2-(3-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]piperazin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 170)

Step 1: Preparation of tert-butyl 3-[8-fluoro-7-[7-fluoro-3-hydroxy-8-(2-triisopropylsilylethynyl)-1-naphthyl]-2-[2-[[4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate

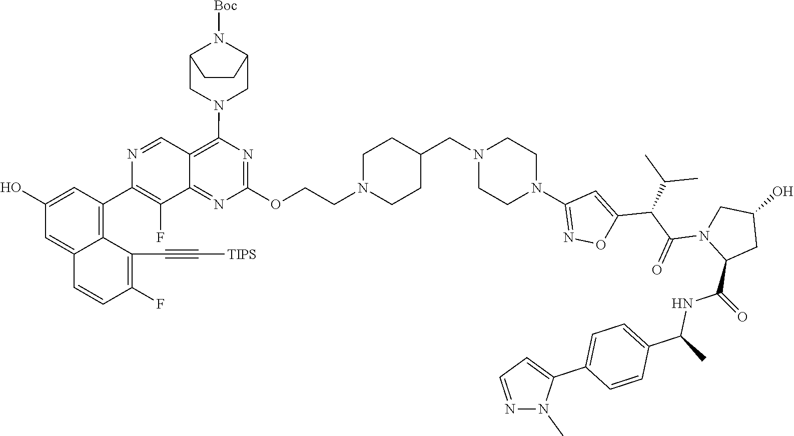

To a solution of tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (300 mg, 0.396 mmol, 1.0 eq) and (2S,4R)-4-hydroxy-1-[(2S)-3-methyl-2-[3-[4-(4-piperidylmethyl) piperazin-1-yl]isoxazol-5-yl]butanoyl]-N-[(1S)-1-[4-(2-methylpyrazol-3-yl)phenyl]ethyl]pyrrolidine-2-carboxamide (257 mg, 0.4 mmol) in CH₂Cl₂ (3 mL) and i-PrOH (3 mL) was added AcOH (0.11 mL, 2 mmol) and 2-methylpyridine borane (212 mg, 2 mmol). The mixture was stirred at 25° C. for 1 h, then trimethylamine was added until pH reached 8 and then concentrated under reduced pressure. The residue was purified by column chromatography (0-10% MeOH in CH₂Cl₂) to afford tert-butyl 3-[8-fluoro-7-[7-fluoro-3-hydroxy-8-(2-triisopropylsilylethynyl)-1-naphthyl]-2-[2-[[4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (420 mg, 76%) as a yellow solid. MS (ESI) m/z: 1389.6 [M+H]⁺.

Step 2: Preparation of tert-butyl 3-[7-(8-ethynyl-7-fluoro-3-hydroxy-1-naphthyl)-8-fluoro-2-[2-[[4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate

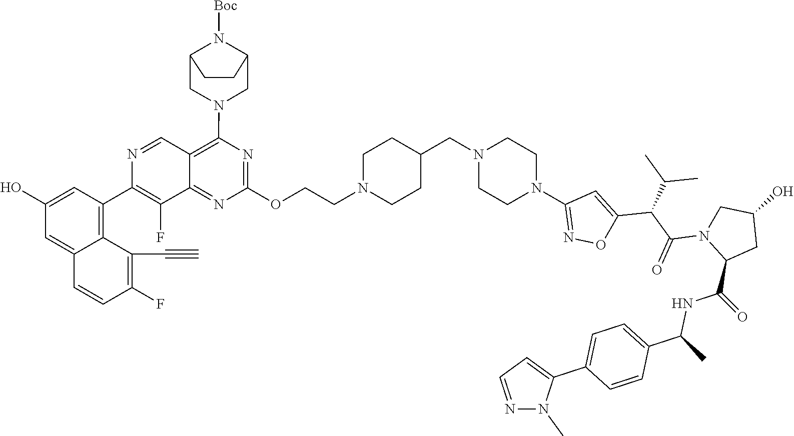 [SMILES: C#Cc1c(F)ccc2cc(O)cc(-c3ncc4c(N5CC6CCC(C5)N6C(=O)OC(C)(C)C)nc(OCCN5CCC(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12]

To a solution of tert-butyl 3-[8-fluoro-7-[7-fluoro-3-hydroxy-8-(2-triisopropylsilylethynyl)-1-naphthyl]-2-[2-[[4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (420 mg, 0.3 mmol) in THF (4 mL) was added TBAF (0.6 mL, 1 M). The mixture was stirred at 25° C. for 10 h. Then TBAF (0.6 mL, 1 M) was added and stirred at 25° C. for another 2 h. The reaction mixture was diluted with ethyl acetate (100 mL). The mixture was washed with water (50 mL×3), brine (100 mL×3), dried over anhydrous Na₂SO₄, filtered and concentrated under reduced pressure to afford tert-butyl 3-[7-(8-ethynyl-7-fluoro-3-hydroxy-1-naphthyl)-8-fluoro-2-[2-[[4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (357 mg) as a yellow solid, which was used directly in the next step. MS (ESI) m/z: 1232.8 [M+H]⁺.

Step 3: Preparation of (2S,4R)-1-[(2S)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

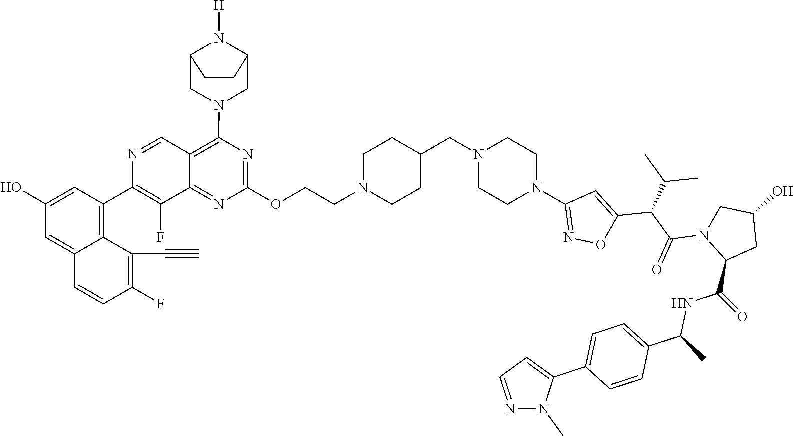 [SMILES: C#Cc1c(F)ccc2cc(O)cc(-c3ncc4c(N5CC6CCC(C5)N6)nc(OCCN5CCC(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12]

A solution of tert-butyl 3-[7-(8-ethynyl-7-fluoro-3-hydroxy-1-naphthyl)-8-fluoro-2-[2-[[4-[[4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (357 mg, 0.29 mmol) in HCOOH (5.0 mL) was stirred at 25° C. for 1 h. The mixture was concentrated, then basified with saturated Na₂CO₃ solution until pH 8 and extracted with CH₂Cl₂/MeOH (20 mL×5, v/v=10/1). The combined organic layers were washed with brine (15 mL×3), dried over anhydrous Na₂SO₄, filtered, and concentrated under reduced pressure. The residue was purification by preparative HPLC (column: Xtimate C18 150*40 mm*10 um; mobile phase: [water (FA)-ACN]; B %: 0%-26%, Gradient time: 36 min), the crude fraction were combined and basified with NaHCO₃ until pH 8, then the suspension was extracted with CH₂Cl₂/MeOH (50 mL×3, V/V=10/1). The combined organic layers were washed with brine (15 mL×3), dried over anhydrous Na₂SO₄, filtered, and concentrated under reduced pressure to afford (2S,4R)-1-[(2S)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (129.3 mg, 39%) as a yellow solid. MS (ESI) m/z: 1132.7 [M+H]⁺; ¹H NMR (400 MHz, CD₃OD) δ 9.02 (s, 1H), 7.88-7.84 (m, 1H), 7.50-7.29 (m, 7H), 7.21 (d, J=2.4 Hz, 1H), 6.34 (d, J=2.0 Hz, 1H), 6.13 (s, 1H), 5.02 (s, 1H), 4.67 (d, J=6.8 Hz, 8H), 4.43 (d, J=2.0 Hz, 1H), 3.90-3.83 (m, 3H), 3.79-3.64 (m, 7H), 3.38 (s, 1H), 3.26-3.16 (m, 6H), 3.04-2.90 (m, 2H), 2.47 (d, J=4.4 Hz, 7H), 2.22 (d, J=6.8 Hz, 3H), 2.00-1.77 (m, 7H), 1.60-1.47 (m, 3H), 1.06 (d, J=6.4 Hz, 3H), 0.92 (d, J=6.8 Hz, 3H).

Exemplary Synthesis of (2S,4R)-1-[(2S)-2-(3-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-4-fluoropiperidin-4-yl)methyl]piperazin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 171)

Step 1: Preparation of tert-butyl 3-[8-fluoro-2-[2-[[4-fluoro-4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate

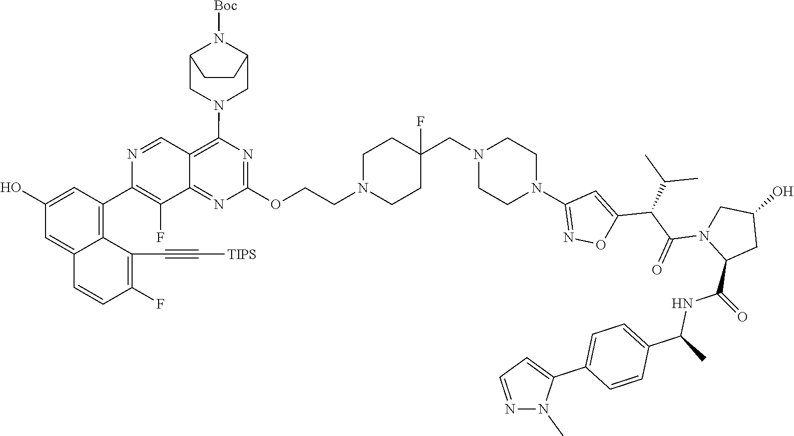

To a mixture of (2S,4R)-1-[(2S)-2-[3-[4-[(4-fluoro-4-piperidyl)methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (263 mg, 0.4 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (300 mg, 0.4 mmol) in CH₂Cl₂ (4 mL) and i-PrOH (4 mL) was added acetic acid (91 uL, 1.6 mmol) and 2-methylpyridine borane (212 mg, 2 mmol) at 25° C. The mixture was stirred at 25° C. for 1 h, then concentrated in vacuum. The pH of the residue was adjusted to 10 by triethylamine. The residue was purified by column chromatography (0-4% then up to 6% methanol (1N NH₃ as additive) in dichloromethane) to afford tert-butyl 3-[8-fluoro-2-[2-[[4-fluoro-4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (403 mg, 72%) as a yellow solid. MS (ESI) m/z: 704.3 [M/2+1]⁺.

Step 2: Preparation of tert-butyl 3-[7-(8-ethynyl-7-fluoro-3-hydroxy-1-naphthyl)-8-fluoro-2-[2-[[4-fluoro-4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate

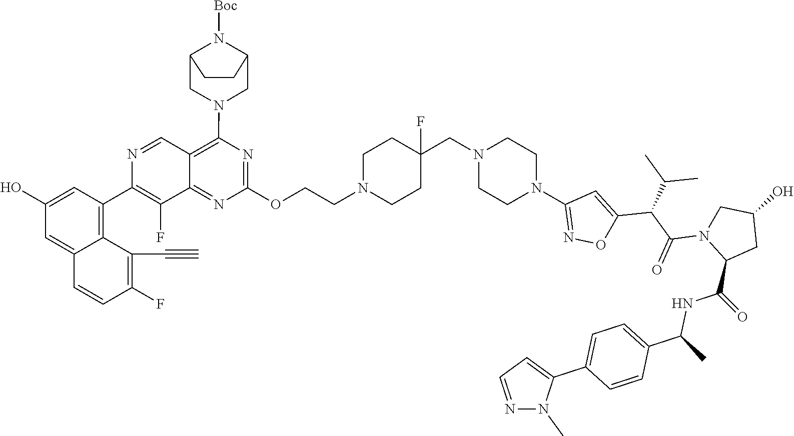 [SMILES: C#Cc1c(F)ccc2cc(O)cc(-c3ncc4c(N5CC6CCC(C5)N6C(=O)OC(C)(C)C)nc(OCCN5CCC(F)(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12]

To a solution of tert-butyl 3-[8-fluoro-2-[2-[[4-fluoro-4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (403 mg, 0.3 mmol) in THF (6 mL) was added TBAF (1 M, 573 uL) at 25° C. The mixture was stirred at 25° C. for 4 h, then concentrated in vacuum. The residue was re-dissolved in ethyl acetate (150 mL), washed with water (15 mL×4) and brine (20 mL), dried over anhydrous Na₂SO₄, filtered, and concentrated to afford tert-butyl 3-[7-(8-ethynyl-7-fluoro-3-hydroxy-1-naphthyl)-8-fluoro-2-[2-[[4-fluoro-4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (356 mg, crude) as a yellow solid. MS (ESI) m/z: 1250.3 [M+1]⁺.

Step 3: Preparation of (2S,4R)-1-[(2S)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-fluoro-4-piperidyl]methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

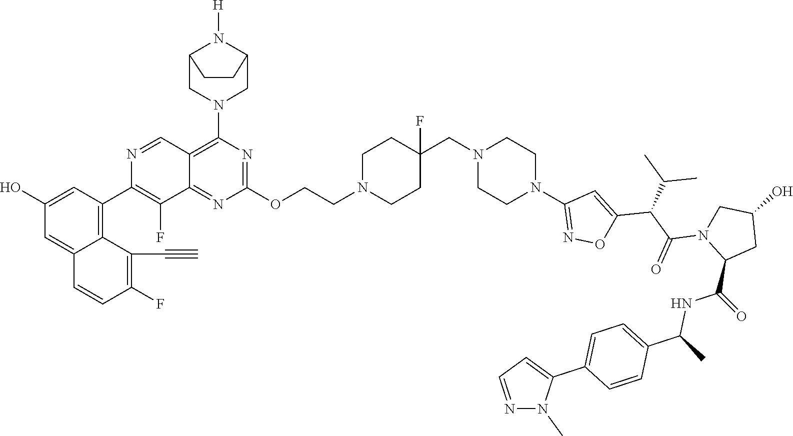 [SMILES: C#Cc1c(F)ccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12]

A mixture of tert-butyl 3-[7-(8-ethynyl-7-fluoro-3-hydroxy-1-naphthyl)-8-fluoro-2-[2-[[4-fluoro-4-[[4-[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (336 mg, 0.3 mmol) in HCOOH (10 mL) was stirred at 25° C. for 2 h, the reaction mixture was concentrated in vacuum. Saturated aqueous Na₂CO₃ was added to adjust the pH to 10. The resulting mixture was extracted with dichloromethane/methanol (120 mL, v/v=10/1). The combined organic phase was washed with brine (30 mL), dried with anhydrous Na₂SO₄, filtered and concentrated. The residue was purified by preparative HPLC (column: Xtimate C18 150*40 mm*10 um; mobile phase: [water (NH₄HCO₃)-ACN]; B %: 22%-62%, 36 min) to afford (2S,4R)-1-[(2S)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-fluoro-4-piperidyl]methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (154.2 mg, 48%) as a yellow solid. MS (ESI) m/z: 1150.5 [M+1]⁺; ¹H NMR (400 MHz, CD₃OD) δ 9.01 (s, 1H), 7.86 (dd, J=5.6, 8.8 Hz, 1H), 7.50-7.36 (m, 5H), 7.36-7.29 (m, 2H), 7.21 (d, J=2.6 Hz, 1H), 6.36-6.31 (m, 1H), 6.13-6.04 (m, 1H), 5.03-4.99 (m, 1H), 4.68-4.56 (m, 7H), 4.43 (s, 1H), 3.88-3.82 (m, 3H), 3.75-3.67 (m, 6H), 3.38 (s, 1H), 3.25-3.21 (m, 1H), 3.18 (s, 3H), 2.96-2.84 (m, 4H), 2.65-2.56 (m, 4H), 2.54-2.47 (m, 3H), 2.43-2.33 (m, 1H), 2.27-2.17 (m, 1H), 1.99-1.80 (m, 7H), 1.74-1.58 (m, 2H), 1.52-1.38 (m, 3H), 1.07-1.01 (m, 3H), 0.96-0.85 (m, 3H).

Exemplary Synthesis of (2S,4R)-1-[(2R)-2-(3-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-4-fluoropiperidin-4-yl)methyl]piperazin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 172)

Step 1: Preparation of tert-butyl 3-[8-fluoro-2-[2-[[4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate

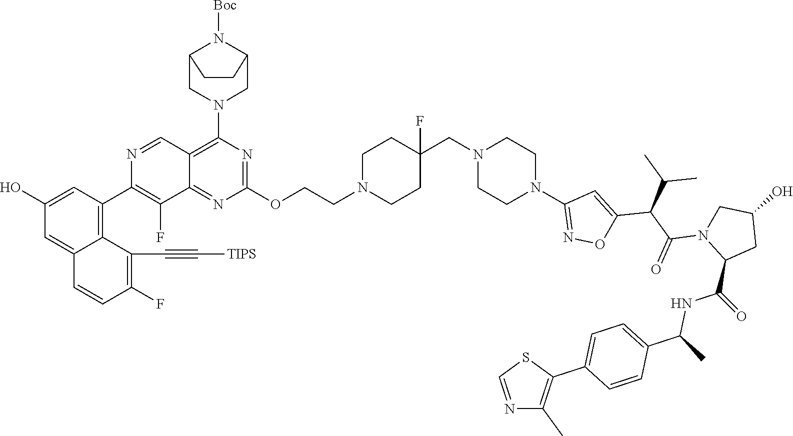

To a mixture of (2S,4R)-1-[(2R)-2-[3-[4-[(4-fluoro-4-piperidyl)methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (248 mg, 0.4 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (276 mg, 0.4 mmol) in CH₂Cl₂ (4 mL) and i-PrOH (4 mL) were added acetic acid (83 uL, 1.5 mmol) and 2-methylpyridine borane (195 mg, 1.8 mmol) at 25° C., the mixture was stirred at 25° C. for 1 h, then concentrated over vacuum at 25° C. The pH of the residue was adjusted to 10 by triethylamine. The residue was purified by column chromatography (0-5% then up to 9% methanol (1N NH₃ as additive) in dichloromethane) to afford tert-butyl 3-[8-fluoro-2-[2-[[4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (439 mg, 85%) as a yellow solid. MS (ESI) m/z: 712.6 [M/2+1]⁺.

Step 2: Preparation of tert-butyl 3-[7-(8-ethynyl-7-fluoro-3-hydroxy-1-naphthyl)-8-fluoro-2-[2-[[4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate

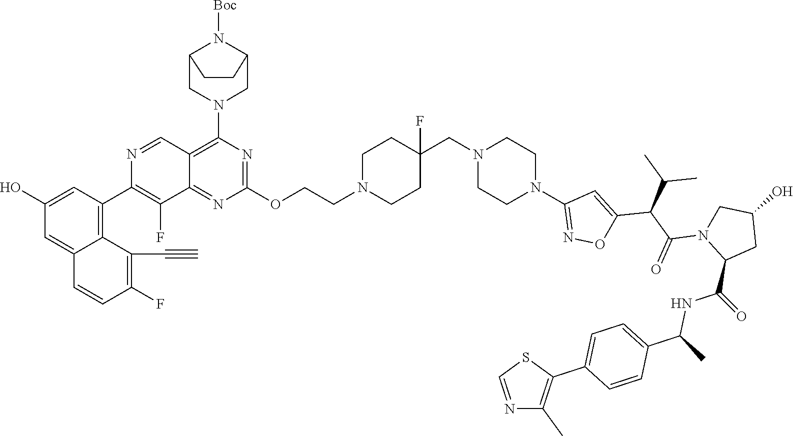 [SMILES: C#Cc1c(F)ccc2cc(O)cc(-c3ncc4c(N5CC6CCC(C5)N6C(=O)OC(C)(C)C)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a solution of tert-butyl 3-[8-fluoro-2-[2-[[4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (439 mg, 0.3 mmol) in THF (6 mL) was added TBAF (1 M, 617 uL) at 25° C. The mixture was stirred at 25° C. for 4 h. Then TBAF (1 M, 308 uL) was added and stirred at 25° C. for 16 h. The reaction mixture was concentrated in vacuum. The residue was re-dissolved in ethyl acetate (120 mL), washed with water (15 mL×5) and brine (20 mL), dried over anhydrous Na₂SO₄, filtered, and concentrated to afford tert-butyl 3-[7-(8-ethynyl-7-fluoro-3-hydroxy-1-naphthyl)-8-fluoro-2-[2-[[4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (339 mg, crude) as a yellow solid. MS (ESI) m/z: 1267.6 [M+1]⁺.

Step 3: Preparation of (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-fluoro-4-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

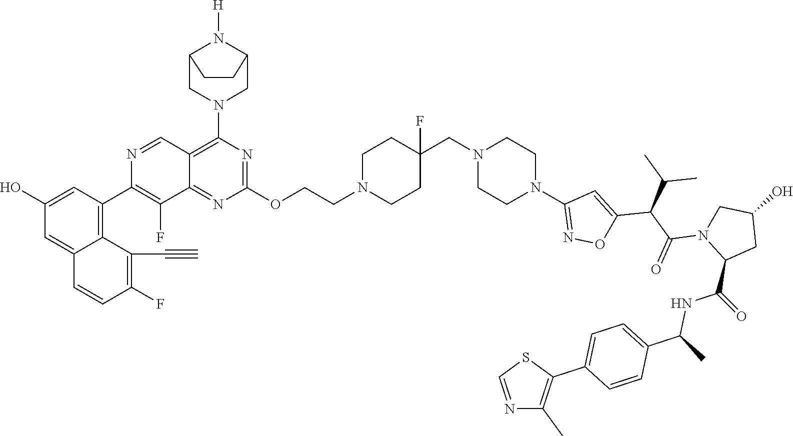 [SMILES: C#Cc1c(F)ccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

A mixture of tert-butyl 3-[7-(8-ethynyl-7-fluoro-3-hydroxy-1-naphthyl)-8-fluoro-2-[2-[[4-fluoro-4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (319 mg, 0.2 mmol) in HCOOH (10 mL) was stirred at 25° C. for 2 h, then concentrated in vacuum. Saturated aqueous Na₂CO₃ was added to adjust the pH to 10. The resulting mixture was extracted with dichloromethane/methanol (120 mL, v/v=10/1), washed with brine (30 mL), dried with anhydrous Na₂SO₄, filtered and concentrated. The residue was purified by preparative HPLC (column: Xtimate C18 150*40 mm*10 um; mobile phase: [water (NH₄HCO₃)-ACN]; B %: 26%-66%, 36 min) to afford (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-fluoro-4-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (158.1 mg, 52%) as a yellow solid. MS (ESI) m/z: 1167.5 [M+1]⁺; ¹H NMR (400 MHz, CD₃OD) δ 9.01 (s, 1H), 8.88 (s, 1H), 7.91-7.82 (m, 1H), 7.47-7.37 (m, 4H), 7.37-7.29 (m, 2H), 7.21 (d, J=2.4 Hz, 1H), 6.11-6.02 (m, 1H), 5.03 (q, J=7.2 Hz, 1H), 4.69-4.58 (m, 6H), 4.53 (s, 1H), 4.46-4.39 (m, 1H), 3.84 (dd, J=4.0, 10.8 Hz, 1H), 3.75-3.68 (m, 4H), 3.65-3.55 (m, 2H), 3.38 (s, 1H), 3.25-3.17 (m, 4H), 2.97-2.85 (m, 4H), 2.64-2.61 (m, 3H), 2.56 (s, 1H), 2.53-2.49 (m, 2H), 2.49-2.47 (m, 3H), 2.42-2.32 (m, 1H), 2.22-2.12 (m, 1H), 2.01-1.78 (m, 8H), 1.77-1.67 (m, 1H), 1.60-1.50 (m, 3H), 1.08-1.02 (m, 3H), 0.93-0.86 (m, 3H).

Exemplary Synthesis of (2S,4R)-1-[(2R)-2-[3-(1′-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-[4,4′-bipiperidin]-1-yl)-1,2-oxazol-5-yl]-3-methylbutanoyl]-4-hydroxy-N-(Compound 176)

Step 1: Preparation of tert-butyl 4-[[1-[5-(1-methoxycarbonyl-2-methyl-propyl)isoxazol-3-yl]-4-piperidyl]piperidine-1-carboxylate

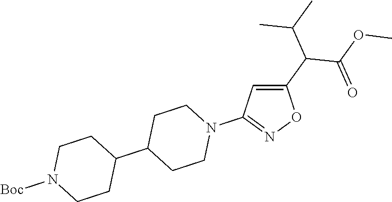 [SMILES: COC(=O)C(c1cc(N2CCC(C3CCN(C(=O)OC(C)(C)C)CC3)CC2)no1)C(C)C]

To a solution of tert-butyl 4-(4-piperidyl)piperidine-1-carboxylate (4 g, 15 mmol, 1 eq) in N,N-dimethylacetamide (40 mL) was dropwise added trimethylamine (4.52 g, 45 mmol, 6.2 mL, 3 eq) at 25° C., then warmed to 120° C. and methyl 3-methyl-2-[3-(1,1,2,2,3,3,4,4,4-nonafluorobutylsulfonyloxy)isoxazol-5-yl]butanoate (7.89 g, 16 mmol, 1.1 eq) in N,N-dimethylacetamide (5 mL) was added dropwise at 120° C. The mixture was stirred at 120° C. for 6.5 h, then cooled to 25° C. The residue was poured into water (300 mL) and stirred for 2 min. The aqueous phase was extracted with ethyl acetate (100 mL×3). The combined organic phase was washed with brine (150 mL×2), dried with anhydrous Sodium sulfate, filtered, and concentrated in vacuum. The residue was purified by prep-HPLC (column: Phenomenex luna C18 (250*70 mm, 10 um); mobile phase: [water (FA)-ACN]; B %: 51%-81%, 26 min) to afford tert-butyl 4-[[1-[5-(1-methoxy carbonyl-2-methyl-propyl)isoxazol-3-yl]-4-piperidyl]piperidine-1-carboxylate (2 g, 30%) as a yellow oil. MS (ESI) m/z: 450.5 [M+1]⁺.

Step 2: Preparation of 2-[3-[4-(1-tert-butoxycarbonyl-4-piperidyl)-1-piperidyl]isoxazol-5-yl]-3-methyl-butanoic acid

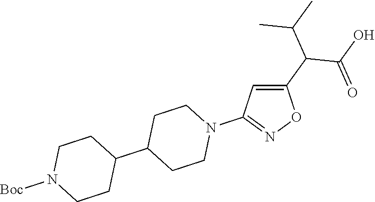 [SMILES: CC(C)C(C(=O)O)c1cc(N2CCC(C3CCN(C(=O)OC(C)(C)C)CC3)CC2)no1]

To a solution of tert-butyl 4-[[1-[5-(1-methoxycarbonyl-2-methyl-propyl)isoxazol-3-yl]-4-piperidyl]piperidine-1-carboxylate (2 g, 4 mmol) in methanol (7 mL) and tetrahydrofuran (7 mL) and water (7 mL) was added lithium hydroxide (533 mg, 22 mmol, 5 eq). The mixture was stirred at 25° C. for 1 h. Aqueous hydrogen chloride (1 M) was added to adjust the pH to 3. The residue was poured into water (20 mL) and stirred for 2 min. The aqueous phase was extracted with ethyl acetate (15 mL×3). The combined organic phase was washed with brine (15 mL×2), dried with anhydrous sodium sulfate, filtered, and concentrated in vacuum to afford 2-[3-[4-(1-tert-butoxycarbonyl-4-piperidyl)-1-piperidyl]isoxazol-5-yl]-3-methyl-butanoic acid (1.69 g, crude) as a white solid, which was used in the next step directly. MS (ESI) m/z: 436.3 [M+1]⁺.

Step 3: Preparation of tert-butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]piperidine-1-carboxylate

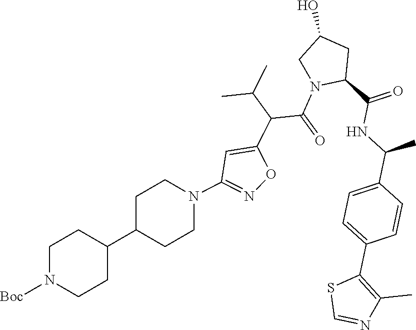 [SMILES: Cc1ncsc1-c1ccc([C@H](C)NC(=O)[C@@H]2C[C@@H](O)CN2C(=O)C(c2cc(N3CCC(C4CCN(C(=O)OC(C)(C)C)CC4)CC3)no2)C(C)C)cc1]

To a solution of 2-[3-[4-(1-tert-butoxycarbonyl-4-piperidyl)-1-piperidyl]isoxazol-5-yl]-3-methyl-butanoic acid (2.8 g, 6 mmol, 1 eq) and (2S,4R)-4-hydroxy-N-[(1S)-1-[4-(4-methylthiazol-5-yl)phenyl]ethyl]pyrrolidine-2-carboxamide hydrochloride (2.37 g, 6 mmol, 1 eq) in N,N-dimethylformamide (30 mL) was added N,N-diisopropylethylamine (4.15 g, 32 mmol, 5.6 mL, 5 eq) and O-(7-Azabenzotriazol-1-yl)-N,N,N′,N′-tetramethyluronium Hexafluorophosphate (2.93 g, 7.7 mmol, 1.2 eq). The mixture was stirred at 25° C. for 0.5 h. The residue was poured into water (200 mL) and stirred for 2 min. The aqueous phase was extracted with ethyl acetate (50 mL×3). The combined organic phase was washed with brine (50 mL×2), dried with anhydrous sodium sulfate, filtered, and concentrated in vacuum. The residue was purified by flash silica gel chromatography (0-100% ethyl acetate/petroleum ether) to afford tert-butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]piperidine-1-carboxylate (4.23 g, 82%) as a yellow solid. MS (ESI) m/z: 749.4 [M+1]⁺; 1H NMR (400 MHz, CDCl₃) δ 8.68 (s, 1H), 7.42-7.32 (m, 4H), 5.90 (d, J=4.6 Hz, 1H), 5.07 (quin, J=7.2 Hz, 1H), 4.97 (q, J=7.2 Hz, 1H), 5.14-4.91 (m, 1H), 4.78-4.54 (dd, J=4.4, 8.4 Hz, 2H), 4.11-4.07 (m, 1H), 3.77-3.49 (m, 5H), 2.80-2.70 (m, 2H), 2.69-2.58 (m, 2H), 2.53 (d, J=2.0 Hz, 3H), 2.51-2.36 (m, 2H), 2.02-1.92 (m, 1H), 1.89-1.56 (m, 8H), 1.50 (d, J=7.2 Hz, 2H), 1.45 (d, J=1.2 Hz, 9H), 1.38 (d, J=7.2 Hz, 1H), 1.31 (s, 1H), 1.24-1.19 (m, 2H), 1.18-1.08 (m, 2H), 1.04 (d, J=6.4 Hz, 3H), 0.93 (d, J=6.6 Hz, 3H).

Step 4: Preparation of tert-butyl 4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]piperidine-1-carboxylate

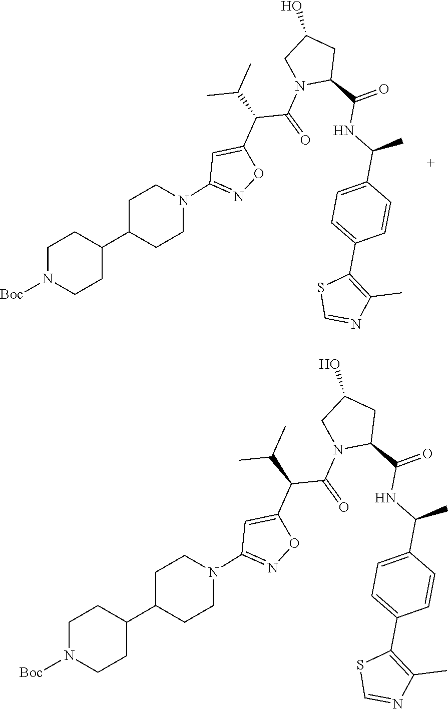

Tert-butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]piperidine-1-carboxylate (4.23 g) was purified by chiral SFC (column: DAICEL CHIRALCEL OD 250×30 mm, I.D., 10 um; mobile phase: isopropanol (0.1% NH₃H₂O) in CO₂ from 35% to 35%; flow rate: 70 mL/min). Fraction one was obtained as tert-butyl 4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]piperidine-1-carboxylate (1.79 g, 85%) as a white solid. ¹H NMR (400 MHz, CDCl₃) δ 8.68 (s, 1H), 7.42-7.37 (m, 2H), 7.36-7.30 (m, 3H), 5.90 (s, 1H), 4.98 (quin, J=7.2 Hz, 1H), 4.79 (dd, J=4.0, 8.4 Hz, 1H), 4.68-4.59 (m, 1H), 4.12 (d, J=1.6 Hz, 2H), 3.73 (dd, J=5.6, 10.4 Hz, 1H), 3.65 (d, J=12.0 Hz, 2H), 3.60-3.51 (m, 2H), 2.81-2.68 (m, 3H), 2.67-2.56 (m, 3H), 2.53 (s, 3H), 2.48-2.39 (m, 1H), 2.05-1.96 (m, 1H), 1.74-1.64 (m, 6H), 1.46 (s, 9H), 1.38 (d, J=7.2 Hz, 3H), 1.30-1.23 (m, 3H), 1.15 (dd, J=8.4, 11.9 Hz, 2H), 1.05 (d, J=6.6 Hz, 3H), 0.96-0.90 (m, 3H). Fraction two was obtained as tert-butyl 4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]piperidine-1-carboxylate (2.07 g, 98%) as a yellow solid. ¹H NMR (400 MHz, CDCl₃) δ 8.67 (s, 1H), 7.59 (d, J=7.6 Hz, 1H), 7.42-7.36 (m, 4H), 5.88 (s, 1H), 5.30 (s, 1H), 5.07 (quin, J=7.2 Hz, 1H), 4.64-4.52 (m, 2H), 4.12 (d, J=7.2 Hz, 2H), 3.79-3.72 (m, 1H), 3.68-3.59 (m, 3H), 3.50-3.46 (m, 1H), 2.74 (t, J=11.6 Hz, 2H), 2.69-2.57 (m, 2H), 2.53 (s, 3H), 2.47-2.37 (m, 2H), 1.96 (m, J=4.4, 8.0, 12.8 Hz, 1H), 1.74-1.63 (m, 4H), 1.50 (d, J=7.2 Hz, 3H), 1.45 (s, 9H), 1.31-1.23 (m, 4H), 1.15 (dd, J=8.4, 12.0 Hz, 2H), 1.04 (d, J=6.4 Hz, 3H), 0.92 (d, J=6.4 Hz, 3H).

Step 5: Preparation of (2S,4R)-4-hydroxy-1-[(2R)-3-methyl-2-[3-[4-(4-piperidyl)-1-piperidyl]isoxazol-5-yl]butanoyl]-N-[(1S)-1-[4-(4-methylthiazol-5-yl)phenyl]ethyl]pyrrolidine-2-carboxamide

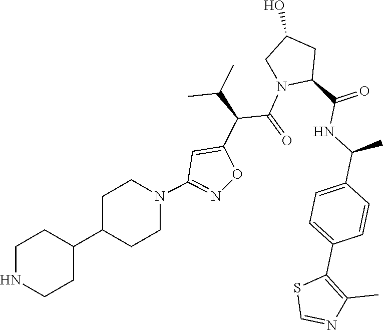 [SMILES: Cc1ncsc1-c1ccc([C@H](C)NC(=O)[C@@H]2C[C@@H](O)CN2C(=O)[C@@H](c2cc(N3CCC(C4CCNCC4)CC3)no2)C(C)C)cc1]

To a solution of tert-butyl 4-[[1-[5-[(1R)-1-[(2S,4R)-4-hydroxy-2-[[(1S)-1-[4-(4-methyl thiazol-5-yl)phenyl]ethyl]carbamoyl]pyrrolidine-1-carbonyl]-2-methyl-propyl]isoxazol-3-yl]-4-piperidyl]piperidine-1-carboxylate (400 mg, 0.5 mmol) in dichloromethane (4 mL) was added trifluoroacetic acid (2.00 g, 17.6 mmol, 1.3 mL). The mixture was stirred at 25° C. for 1 h, then concentrated under reduced pressure to afford (2S,4R)-4-hydroxy-1-[(2R)-3-methyl-2-[3-[4-(4-piperidyl)-1-piperidyl]isoxazol-5-yl]butanoyl]-N-[(1S)-1-[4-(4-methylthiazol-5-yl)phenyl]ethyl]pyrrolidine-2-carboxamide trifluoroacetate (346 mg, crude) as a colorless oil, which was used in the next step directly. MS (ESI) m/z: 649.3 [M+1]⁺.

Step 6: Preparation of tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]-1-piperidyl]ethoxy]pyrido[4,3-d]pyrimidin-4-yl]-3,8-diazabicyclo[3.2.1]octane-8-carboxylate

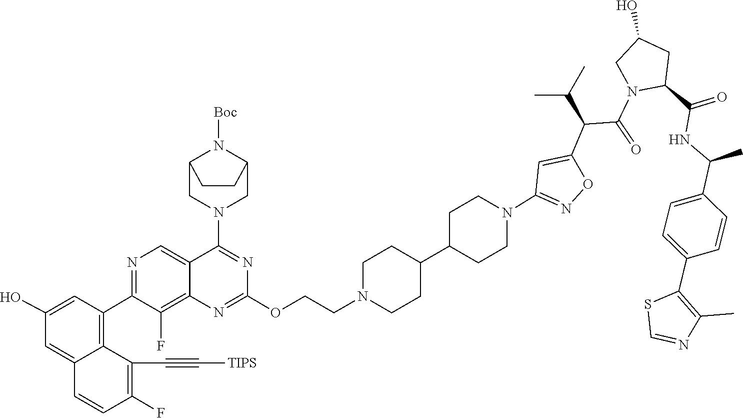

To a solution of (2S,4R)-4-hydroxy-1-[(2R)-3-methyl-2-[3-[4-(4-piperidyl)-1-piperidyl]isoxazol-5-yl]butanoyl]-N-[(1S)-1-[4-(4-methylthiazol-5-yl)phenyl]ethyl]pyrrolidine-2-carboxamide (346 mg, 0.5 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (485 mg, 0.6 mmol) in isopropanol (3 mL) and dichloromethane (3 mL) was added N,N-diisopropylethylamine (345 mg, 2.7 mmol, 0.5 mL) and stirred for 0.5 h. Sodium triacetoxyborohydride (226 mg, 1 mmol) was added and the mixture was stirred at 25° C. for 0.5 h. The reaction was poured into water (15 mL) and stirred for 1 min. The aqueous phase was extracted with ethyl acetate (10 mL×3). The combined organic phase was washed with brine (10 mL×2), dried with anhydrous Sodium sulfate, filtered and concentrated in vacuum. The residue was purified by flash silica gel chromatography (0-15% dichloromethane/methanol ether) to afford tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]-1-piperidyl]ethoxy]pyrido[4,3-d]pyrimidin-4-yl]-3,8-diazabicyclo[3.2.1]octane-8-carboxylate (684 mg, 92%) as a white solid. MS (ESI) m/z: 1390.8 [M+1]⁺.

Step 7: Preparation of tert-butyl 3-[7-(8-ethynyl-7-fluoro-3-hydroxy-1-naphthyl)-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]-1-piperidyl]ethoxy]pyrido[4,3-d]pyrimidin-4-yl]-3,8-diazabicyclo[3.2.1]octane-8-carboxylate

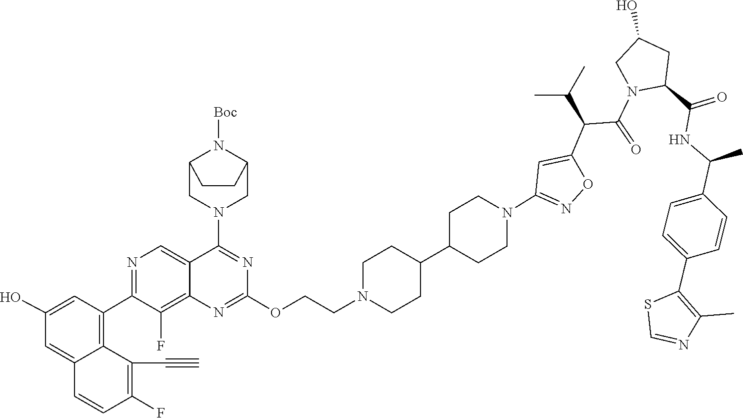 [SMILES: C#Cc1c(F)ccc2cc(O)cc(-c3ncc4c(N5CC6CCC(C5)N6C(=O)OC(C)(C)C)nc(OCCN5CCC(C6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a solution of tert-butyl 3-[8-fluoro-7-[7-fluoro-3-hydroxy-8-(2-triisopropylsilylethynyl)-1-naphthyl]-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]-1-piperidyl]ethoxy]pyrido[4,3-d]pyrimidin-4-yl]-3,8-diazabicyclo[3.2.1]octane-8-carboxylate (684 mg, 0.5 mmol) in N,N-dimethylformamide (7 mL) was added cesium fluoride (1.49 g, 9.8 mmol). The mixture was stirred at 25° C. for 2 h, then poured into water (50 mL) and stirred for 2 min. The aqueous phase was extracted with ethyl acetate (10 mL×3). The combined organic phase was washed with brine (10 mL×2), dried with anhydrous sodium sulfate, filtered and concentrated in vacuum. The residue was purified by prep-TLC (silicon dioxide, dichloromethane:methanol=10:1) to afford tert-butyl 3-[7-(8-ethynyl-7-fluoro-3-hydroxy-1-naphthyl)-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]-1-piperidyl]ethoxy]pyrido[4,3-d]pyrimidin-4-yl]-3,8-diazabicyclo[3.2.1]octane-8-carboxylate (500 mg, 82%) as a white solid. MS (ESI) m/z: 1234.7 [M+1]⁺.

Step 8: Preparation of (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

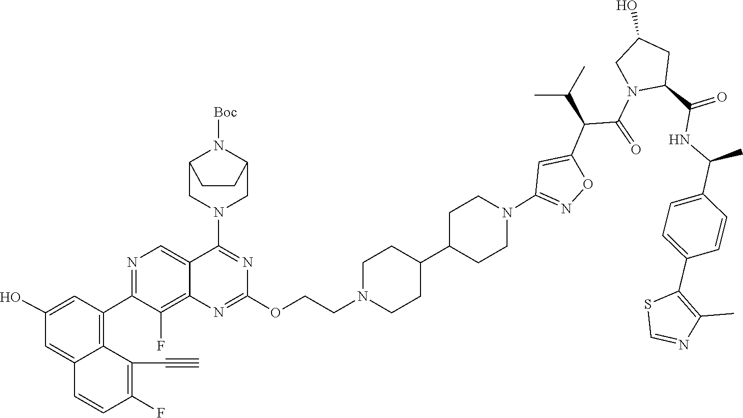 [SMILES: C#Cc1c(F)ccc2cc(O)cc(-c3ncc4c(N5CC6CCC(C5)N6C(=O)OC(C)(C)C)nc(OCCN5CCC(C6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a solution of tert-butyl 3-[7-(8-ethynyl-7-fluoro-3-hydroxy-1-naphthyl)-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]-1-piperidyl]ethoxy]pyrido[4,3-d]pyrimidin-4-yl]-3,8-diazabicyclo[3.2.1]octane-8-carboxylate (500 mg, 0.4 mmol) in dichloromethane (4.5 mL) was added trifluoroacetic acid (2.31 g, 20 mmol, 1.5 mL). The mixture was stirred at 25° C. for 1 h, then concentrated under reduced pressure. To the mixture was added N,N-diisopropylethylamine (1 mL) to adjust the pH to 8. The residue was purified by prep-HPLC (column: Waters Xbridge Prep OBD C18 150*40 mm*10 um; mobile phase: [water (NH₄HCO₃)-ACN]; B %: 23%-53%, 10 min) to afford (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (209.5 mg, 44%) as a yellow solid. MS (ESI) m/z: 568.0 [M/2+1]⁺; 1H NMR (400 MHz, DMSO-d₆) δ 10.19-10.11 (m, 1H), 9.03 (s, 1H), 8.99-8.97 (m, 1H), 8.39 (d, J=7.6 Hz, 1H), 7.97 (dd, J=6.0, 9.2 Hz, 1H), 7.49-7.34 (m, 8H), 7.17 (d, J=2.4 Hz, 1H), 6.15-5.94 (m, 1H), 5.10 (d, J=3.2 Hz, 1H), 4.93-4.88 (m, 1H), 4.48-4.41 (m, 3H), 4.38-4.31 (m, 2H), 4.28 (d, J=6.0 Hz, 2H), 3.93 (s, 1H), 3.69 (d, J=5.2 Hz, 1H), 3.63 (d, J=11.6 Hz, 3H), 3.58-3.52 (m, 5H), 3.02-2.91 (m, 3H), 2.45 (s, 3H), 2.23 (b s, 2H), 2.04-1.96 (m, 2H), 1.92 (d, J=13.5 Hz, 2H), 1.82-1.76 (m, 1H), 1.65 (s, 4H), 1.37 (d, J=7.0 Hz, 3H), 1.21-1.12 (m, 6H), 1.05 (t, J=6.8 Hz, 2H), 0.94 (d, J=6.4 Hz, 3H), 0.82 (d, J=6.4 Hz, 1H), 0.78 (d, J=6.8 Hz, 3H).

Exemplary Synthesis of (2S,4R)-1-[(2S)-2-[3-(1′-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-[4,4′-bipiperidin]-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 177)

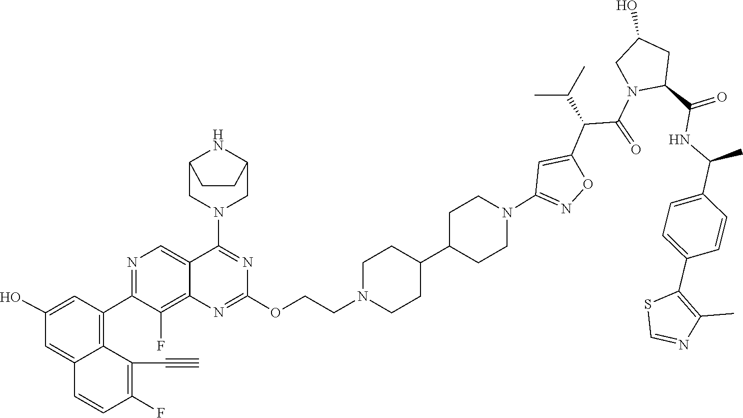 [SMILES: C#Cc1c(F)ccc2cc(O)cc(-c3ncc4c(N5CC6CCC(C5)N6)nc(OCCN5CCC(C6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was made in an analogous manner to (2S,4R)-1-[(2R)-2-[3-(1′-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-[4,4′-bipiperidin]-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide, starting from tert-butyl 4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]piperidine-1-carboxylate and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate. (yellow solid). MS (ESI) m/z: 1134.7 [M+1]⁺; 1H NMR (400 MHz, DMSO-d₆) δ 10.20-10.05 (m, 1H), 9.03 (s, 1H), 9.00-8.97 (m, 1H), 8.19-7.95 (m, 2H), 7.54-7.24 (m, 8H), 7.17 (d, J=2.4 Hz, 1H), 6.12 (s, 1H), 5.12-4.99 (m, 1H), 4.97-4.85 (m, 1H), 4.48-4.38 (m, 4H), 4.32-4.25 (m, 2H), 3.93 (s, 1H), 3.69-3.56 (m, 5H), 3.54 (s, 3H), 3.46-3.41 (m, 1H), 2.96 (dd, J=3.6, 5.4 Hz, 2H), 2.89 (s, 1H), 2.73 (s, 1H), 2.44 (s, 3H), 2.30-2.19 (m, 2H), 2.14 (s, 1H), 2.03 (s, 1H), 1.96-1.86 (m, 3H), 1.84-1.76 (m, 1H), 1.65 (s, 5H), 1.45 (d, J=6.8 Hz, 1H), 1.34 (d, J=7.2 Hz, 2H), 1.20-1.11 (m, 5H), 1.05 (t, J=7.2 Hz, 1H), 0.95 (d, J=6.8 Hz, 3H), 0.81 (d, J=6.8 Hz, 3H), 0.74 (d, J=6.8 Hz, 1H).

Exemplary Synthesis of (2S,4R)-1-[(2S)-2-{3-[[3-({1-[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) azetidin-3-yl]piperidin-4-yl}methyl) azetidin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 180)

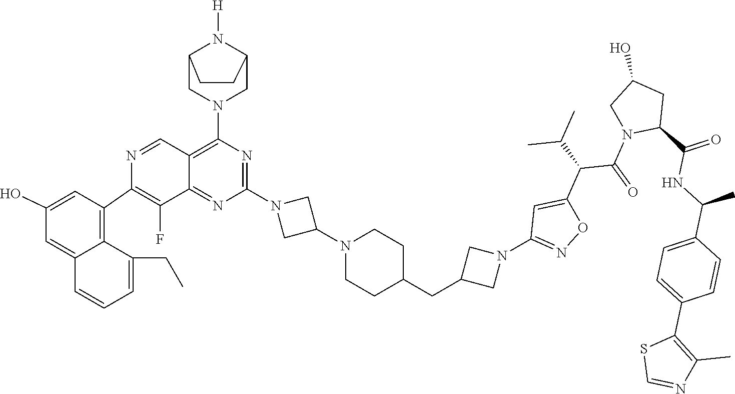 [SMILES: CCc1cccc2cc(O)cc(-c3ncc4c(N5CC6CCC(C5)N6)nc(N5CC(N6CCC(CC7CN(c8cc([C@@H](C(=O)N9C[C@H](O)C[C@H]9C(=O)N[C@@H](C)c9ccc(-c%10scnc%10C)cc9)C(C)C)on8)C7)CC6)C5)nc4c3F)c12]

The title compound was prepared in an analogous manner to (2S,4R)-1-[(2R)-2-{3-[[3-({1-[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) azetidin-3-yl]piperidin-4-yl}methyl) azetidin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide starting from tert-butyl 3-[7-[8-ethyl-3-(methoxymethoxy)-1-naphthyl]-8-fluoro-2-(3-oxoazetidin-1-yl)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). MS (ESI) m/z: 1117.7 [M+1]⁺; 1H NMR (400 MHz, CD₃OD) δ 8.93-8.85 (m, 1H), 8.81 (s, 1H), 8.47-8.34 (m, 1H), 7.62 (d, J=8.0 Hz, 1H), 7.50-7.32 (m, 5H), 7.28 (d, J=2.8 Hz, 1H), 7.16 (d, J=7.2 Hz, 1H), 6.99 (d, J=2.4 Hz, 1H), 5.92-5.77 (m, 1H), 5.05-4.95 (m, 1H), 4.72-4.53 (m, 4H), 4.47-4.40 (m, 1H), 4.39-4.27 (m, 2H), 4.17-3.97 (m, 6H), 3.84-3.50 (m, 7H), 3.03-2.80 (m, 3H), 2.51-2.46 (m, 3H), 2.43-2.28 (m, 4H), 2.17-1.91 (m, 7H), 1.78-1.68 (m, 2H), 1.66-1.57 (m, 2H), 1.49 (d, J=7.2 Hz, 3H), 1.42-1.19 (m, 3H), 1.05 (d, J=6.8 Hz, 3H), 0.97-0.82 (m, 6H).

Exemplary Synthesis of (2S,4R)-1-[(2S)-2-[3-(3-{[(3R)-3-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-4-methylpiperazin-1-yl]methyl}azetidin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (Compound 181)

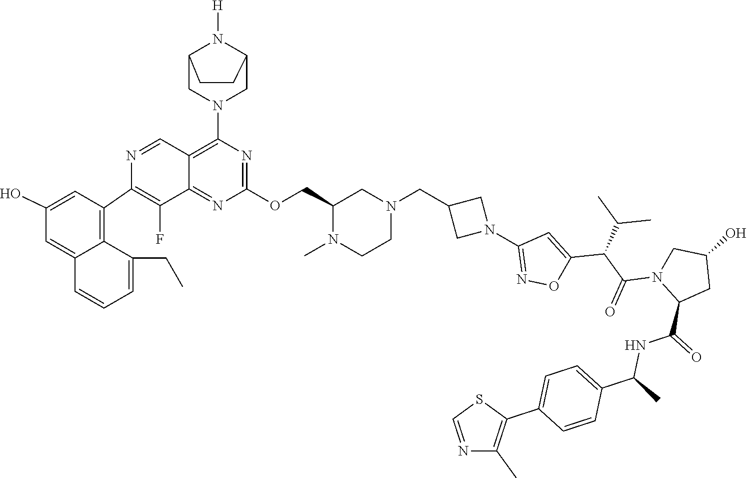 [SMILES: CCc1cccc2cc(O)cc(-c3ncc4c(N5CC6CCC(C5)N6)nc(OC[C@H]5CN(CC6CN(c7cc([C@@H](C(=O)N8C[C@H](O)C[C@H]8C(=O)N[C@@H](C)c8ccc(-c9scnc9C)cc8)C(C)C)on7)C6)CCN5C)nc4c3F)c12]

The title compound was made analogously to (2S,4R)-1-[(2R)-2-[3-(3-{[(3R)-3-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-4-methylpiperazin-1-yl]methyl}azetidin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide, starting from tert-butyl 3-[7-[8-ethyl-3-(methoxymethoxy)-1-naphthyl]-8-fluoro-2-[[(2R)-1-methylpiperazin-2-yl]methoxy]pyrido[4,3-d]pyrimidin-4-yl]-3,8-diazabicyclo[3.2.1]octane-8-carboxylate and (2S,4R)-1-[(2S)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide. (white solid). MS (ESI) m/z: 1107.6 [M+1]⁺; ¹H NMR (400 MHz, CD₃OD) δ 9.06-9.00 (m, 1H), 8.88-8.81 (m, 1H), 7.60 (d, J=8.4 Hz, 1H), 7.46-7.21 (m, 6H), 7.13 (d, J=6.8 Hz, 1H), 7.00 (t, J=2.4 Hz, 1H), 5.93-5.70 (m, 1H), 5.01-4.93 (m, 1H), 4.64-4.55 (m, 6H), 4.49-4.38 (m, 2H), 3.98 (d, J=8.0 Hz, 2H), 3.71-3.56 (m, 8H), 3.03-2.91 (m, 2H), 2.82 (d, J=12.0 Hz, 1H), 2.75-2.69 (m, 1H), 2.67-2.57 (m, 3H), 2.45-2.24 (m, 11H), 1.94 (ddd, J=4.8, 8.2, 13.2 Hz, 1H), 1.88-1.72 (m, 4H), 1.57-1.43 (m, 3H), 1.02 (d, J=6.4 Hz, 3H), 0.96-0.79 (m, 7H).

Exemplary Synthesis of (2S,4R)-1-[(2R)-2-(3-{3-[(1-{[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) azetidin-3-yl]methyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 182)

Step 1: Preparation of tert-butyl 3-[7-(8-ethyl-3-hydroxy-1-naphthyl)-8-fluoro-2-[3-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]methyl]azetidin-1-yl]pyrido[4,3-d]pyrimidin-4-yl]-3,8-diazabicyclo[3.2.1]octane-8-carboxylate

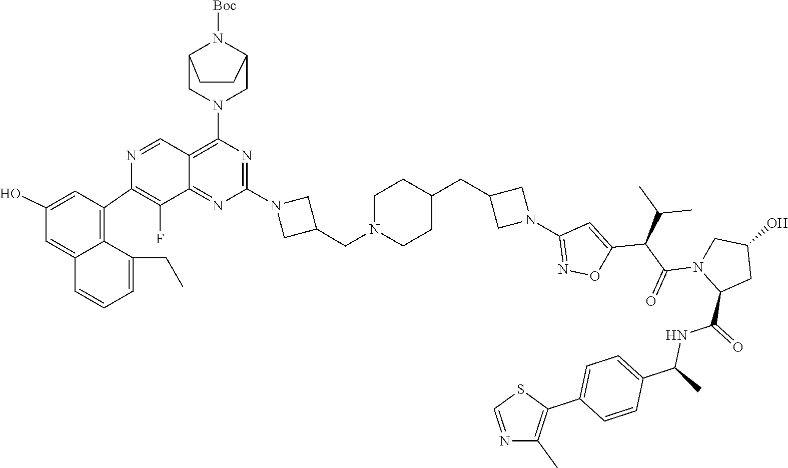 [SMILES: CCc1cccc2cc(O)cc(-c3ncc4c(N5CC6CCC(C5)N6C(=O)OC(C)(C)C)nc(N5CC(CN6CCC(CC7CN(c8cc([C@H](C(=O)N9C[C@H](O)C[C@H]9C(=O)N[C@@H](C)c9ccc(-c%10scnc%10C)cc9)C(C)C)on8)C7)CC6)C5)nc4c3F)c12]

To a solution of tert-butyl 3-[7-(8-ethyl-3-hydroxy-1-naphthyl)-8-fluoro-2-(3-formylazetidin-1-yl)pyrido[4,3-d]pyrimidin-4-yl]-3,8-diazabicyclo[3.2.1]octane-8-carboxylate (205 mg, 97.58% purity, 0.3 mmol) and (2S,4R)-4-hydroxy-1-[(2R)-3-methyl-2-[3-[[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide (230 mg, 0.3 mmol) in CH₂Cl₂ (3 mL) and i-PrOH (3 mL) were added 2-methylpyridine borane (175 mg, 1.6 mmol) and acetic acid (93 uL, 1.6 mmol). The mixture was stirred at 25° C. for 1 h. Triethylamine was added until pH 8, then concentrated under reduced pressure. The residue was purified by column chromatography (0-15% methanol in dichloromethane) to afford tert-butyl 3-[7-(8-ethyl-3-hydroxy-1-naphthyl)-8-fluoro-2-[3-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]methyl]azetidin-1-yl]pyrido[4,3-d]pyrimidin-4-yl]-3,8-diazabicyclo[3.2.1]octane-8-carboxylate (385 mg, 91%) as a yellow solid. MS (ESI) m/z: 1231.6 [M+1]⁺.

Step 2: Preparation of (2S,4R)-1-[(2R)-2-[3-[[3-[[1-[[1-[4-(3,8-diazabicyclo[3.2.1]octan-3-yl)-7-(8-ethyl-3-hydroxy-1-naphthyl)-8-fluoro-pyrido[4,3-d]pyrimidin-2-yl]azetidin-3-yl]methyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

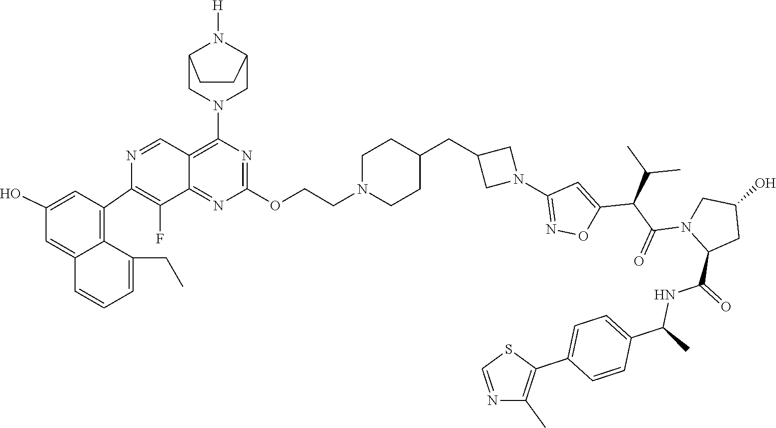 [SMILES: CCc1cccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

To a solution of tert-butyl 3-[7-(8-ethyl-3-hydroxy-1-naphthyl)-8-fluoro-2-[3-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]methyl]azetidin-1-yl]pyrido[4,3-d]pyrimidin-4-yl]-3,8-diazabicyclo[3.2.1]octane-8-carboxylate (385 mg, 0.3 mmol) in CH₂Cl₂ (6 mL) was added TFA (1.9 mL, 26 mmol). The mixture was stirred at 25° C. for 1 h. NaHCO₃ was added until pH reached 8, extracted with methanol:dichloromethane=1:10, concentrated in vacuum and purified by preparative HPLC (column: Xtimate C18 150*40 mm*10 um; mobile phase: [water (FA)-ACN]; B %: 0%-36%, 36 min) to afford (2S,4R)-1-[(2R)-2-[3-[[3-[[1-[1-[4-(3,8-diazabicyclo[3.2.1]octan-3-yl)-7-(8-ethyl-3-hydroxy-1-naphthyl)-8-fluoro-pyrido[4,3-d]pyrimidin-2-yl]azetidin-3-yl]methyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (116.3 mg, 34%) as a white solid. MS (ESI) m/z: 1131.7 [M+1]⁺; 1H NMR (400 MHz, CD₃OD) δ 8.88 (s, 1H), 8.81 (s, 1H), 8.51 (d, J=4.8 Hz, 1H), 7.63 (d, J=8.0 Hz, 1H), 7.47-7.34 (m, 5H), 7.28 (d, J=2.4 Hz, 1H), 7.16 (d, J=6.8 Hz, 1H), 6.99 (d, J=2.4 Hz, 1H), 5.89-5.78 (m, 1H), 5.07-5.01 (m, 1H), 4.64-4.48 (m, 5H), 4.46-4.39 (m, 3H), 4.06 (t, J=7.2 Hz, 2H), 4.02-3.92 (m, 4H), 3.87-3.81 (m, 1H), 3.77-3.55 (m, 6H), 3.26-3.18 (m, 2H), 3.16-3.07 (m, 3H), 2.95-2.85 (m, 1H), 2.59-2.50 (m, 2H), 2.48 (s, 3H), 2.41-2.29 (m, 3H), 2.23-2.15 (m, 1H), 2.06-1.98 (m, 4H), 1.83 (d, J=12.0 Hz, 2H), 1.68 (s, 2H), 1.61-1.51 (m, 3H), 1.46-1.36 (m, 2H), 1.05 (d, J=6.4 Hz, 3H), 0.94-0.86 (m, 6H).

Exemplary Synthesis of (2S,4R)-1-[(2R)-2-{3-[(3aR,7aR)-5-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-octahydro-1H-pyrrolo[3,4-c]pyridin-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 183)

Step 1: Preparation of tert-butyl 3-[2-[2-[(3aR,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridin-5-yl]ethoxy]-8-fluoro-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate

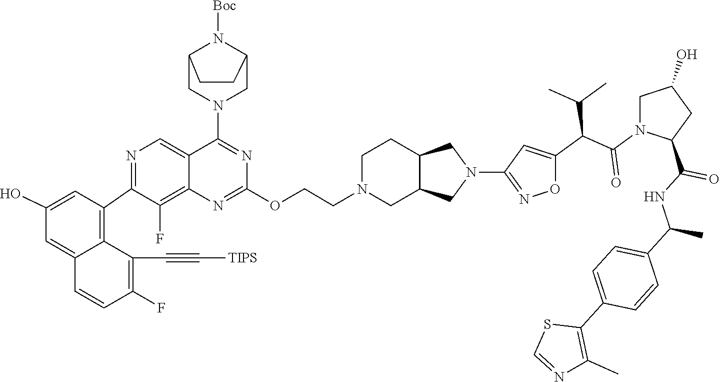

To a solution of (2S,4R)-1-[(2R)-2-[3-[(3aR,7aR)-1,3,3a,4,5,6,7,7a-octahydropyrrolo[3,4-c]pyridin-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide trifluoroacetate (440 mg, 0.7 mmol) in dichloromethane (4 mL) and isopropanol (4 mL) was added N,N-diisopropylethylamine (237 mg, 1.8 mmol, 0.3 mL), then tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (596 mg, 0.8 mmol) was added. The solution was stirred at 25° C. for 10 min. To the solution was added sodium triacetoxyborohydride (388 mg, 1.8 mmol), the reaction was stirred at 25° C. for 0.5 h. The solution was diluted with water (40 mL) and extracted with dichloromethane (30 mL×2). The combined organic layer was dried over anhydrous sodium sulfate, filtered, and concentrated in vacuum. The residue was purified by silica gel column chromatography (petroleum ether/ethyl acetate=1/1 to dichloromethane/methanol=10/1) to afford tert-butyl 3-[2-[2-[(3aR,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridin-5-yl]ethoxy]-8-fluoro-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (800 mg, 97%) as a light yellow solid. MS (ESI) m/z: 1348.9 [M+1]⁺.

Step 2: Preparation of tert-butyl 3-[2-[2-[(3aR,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridin-5-yl]ethoxy]-7-(8-ethynyl-7-fluoro-3-hydroxy-1-naphthyl)-8-fluoro-pyrido[4,3-d]pyrimidin-4-yl]-3,8-diazabicyclo[3.2.1]octane-8-carboxylate

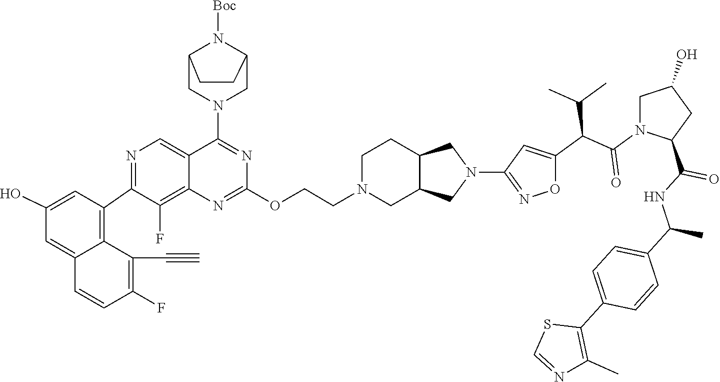 [SMILES: C#Cc1c(F)ccc2cc(O)cc(-c3ncc4c(N5CC6CCC(C5)N6C(=O)OC(C)(C)C)nc(OCCN5CC[C@H]6CN(c7cc([C@H](C(=O)N8C[C@H](O)C[C@H]8C(=O)N[C@@H](C)c8ccc(-c9scnc9C)cc8)C(C)C)on7)C[C@H]6C5)nc4c3F)c12]

To a solution of tert-butyl 3-[2-[2-[(3aR,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridin-5-yl]ethoxy]-8-fluoro-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (800 mg, 0.6 mmol) in N,N-dimethylformamide (12 mL) was added cesium fluoride (1.80 g, 12 mmol), the reaction mixture was stirred at 25° C. for 2 h. The solution was diluted with water (20 mL) and extracted with ethyl acetate (20 mL×3). The combined organic layer was washed with water (20 mL×2). The organic layer was dried over anhydrous sodium sulfate, filtered, and concentrated under vacuum. The residue was purified by prep-TLC (dichloromethane/methanol=8/1) to afford tert-butyl 3-[2-[2-[(3aR,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridin-5-yl]ethoxy]-7-(8-ethynyl-7-fluoro-3-hydroxy-1-naphthyl)-8-fluoro-pyrido[4,3-d]pyrimidin-4-yl]-3,8-diazabicyclo[3.2.1]octane-8-carboxylate (440 mg, 62%) as a light yellow solid. MS (ESI) m/z: 1192.8 [M+1]⁺; 1H NMR (400 MHz, DMSO-d₆) δ 10.16 (s, 1H), 9.16-9.03 (m, 1H), 8.98 (s, 1H), 8.40 (dd, J=2.8, 7.5 Hz, 1H), 8.01-7.90 (m, 1H), 7.57-7.31 (m, 6H), 7.19 (dd, J=2.6, 4.1 Hz, 1H), 5.89-5.70 (m, 1H), 5.16-4.98 (m, 1H), 4.91 (quin, J=7.0 Hz, 1H), 4.65-4.53 (m, 1H), 4.50-4.33 (m, 3H), 4.28 (s, 3H), 4.09 (q, J=5.2 Hz, 1H), 3.97-3.88 (m, 1H), 3.76-3.58 (m, 3H), 3.54 (dd, J=2.9, 9.9 Hz, 1H), 3.46-3.38 (m, 1H), 3.32-3.21 (m, 2H), 3.17 (d, J=5.0 Hz, 3H), 3.14-3.01 (m, 2H), 2.70 (t, J=11.8 Hz, 2H), 2.47-2.43 (m, 3H), 2.40-2.27 (m, 3H), 2.25-2.14 (m, 2H), 2.01 (t, J=9.8 Hz, 1H), 1.88-1.65 (m, 6H), 1.49-1.43 (m, 9H), 1.38 (d, J=7.0 Hz, 3H), 0.99-0.90 (m, 3H), 0.84-0.71 (m, 3H).

Step 3: Preparation of (2S,4R)-1-[(2R)-2-[3-[(3aR,7aR)-5-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-3,3a,4,6,7,7a-hexahydro-1H-pyrrolo[3,4-c]pyridin-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

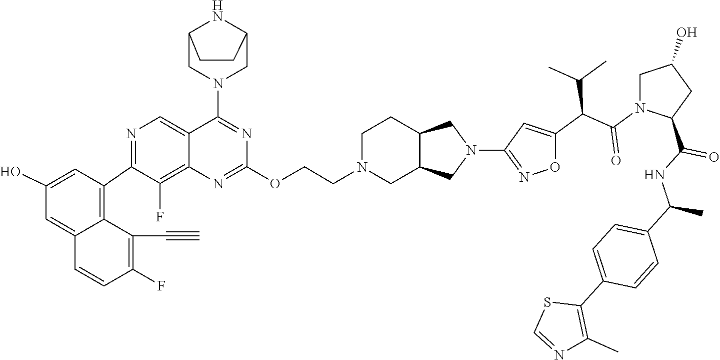 [SMILES: C#Cc1c(F)ccc2cc(O)cc(-c3ncc4c(N5CC6CCC(C5)N6)nc(OCCN5CC[C@H]6CN(c7cc([C@H](C(=O)N8C[C@H](O)C[C@H]8C(=O)N[C@@H](C)c8ccc(-c9scnc9C)cc8)C(C)C)on7)C[C@H]6C5)nc4c3F)c12]

To a solution of tert-butyl 3-[2-[2-[(3aR,7aR)-2-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3,3a,4,6,7,7a-hexahydro-1H-pyrrolo[3,4-c]pyridin-5-yl]ethoxy]-7-(8-ethynyl-7-fluoro-3-hydroxy-1-naphthyl)-8-fluoro-pyrido[4,3-d]pyrimidin-4-yl]-3,8-diazabicyclo[3.2.1]octane-8-carboxylate (440 mg, 0.3 mmol) in dichloromethane (5 mL) was added trifluoroacetic acid (1.54 g, 13 mmol, 1 mL), the reaction mixture was stirred at 25° C. for 0.5 h. The solution was concentrated under vacuum. The pH of the residue was adjusted to 7 by N,N-diisopropylethylamine and purified by prep-HPLC (column: Waters Xbridge 150*25 mm*5 um; mobile phase: [water (NH₄HCO₃)-ACN]; B %: 20%-50%, 10 min) to afford (2S,4R)-1-[(2R)-2-[3-[(3aR,7aR)-5-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-3,3a,4,6,7,7a-hexahydro-1H-pyrrolo[3,4-c]pyridin-2-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (257.6 mg, 60%) as a yellow solid. MS (ESI) m/z: 1092.7 [M+1]⁺; 1H NMR (400 MHz, MeOD-d₄) δ 9.01 (d, J=13.2 Hz, 1H), 8.91-8.78 (m, 1H), 7.89-7.80 (m, 1H), 7.46-7.37 (m, 4H), 7.36-7.30 (m, 2H), 7.30-7.25 (m, 1H), 7.21 (d, J=2.4 Hz, 1H), 5.96-5.72 (m, 1H), 5.15-4.95 (m, 1H), 4.69-4.54 (m, 5H), 4.50 (t, J=8.0 Hz, 1H), 4.45-4.33 (m, 1H), 3.87-3.74 (m, 1H), 3.74-3.63 (m, 4H), 3.62-3.47 (m, 2H), 3.40-3.34 (m, 2H), 3.22-3.12 (m, 2H), 2.88-2.77 (m, 2H), 2.74-2.57 (m, 3H), 2.53 (dd, J=5.6, 11.6 Hz, 1H), 2.47 (d, J=2.0 Hz, 3H), 2.45 (s, 1H), 2.41-2.26 (m, 3H), 2.21-2.09 (m, 1H), 1.96-1.75 (m, 6H), 1.68-1.55 (m, 2H), 1.52 (d, J=7.2 Hz, 3H), 1.03 (dd, J=4.0, 6.4 Hz, 3H), 0.94-0.85 (m, 3H).

Exemplary Synthesis of (2S,4R)-1-[(2S)-2-(3-{3-[(1-{[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) azetidin-3-yl]methyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 189)

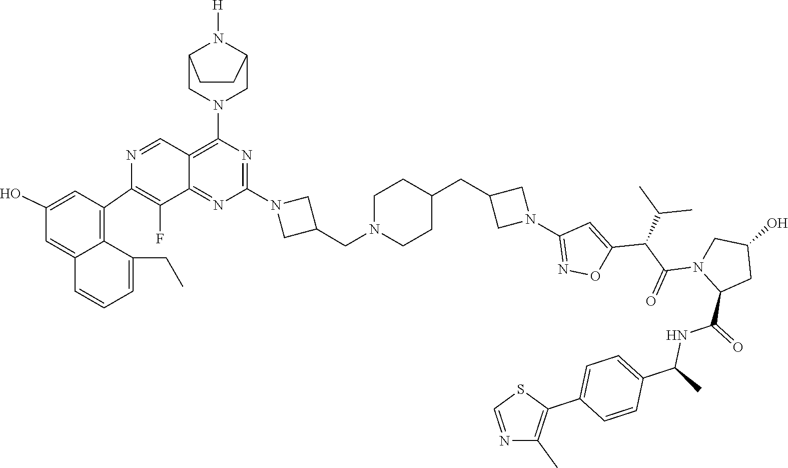 [SMILES: CCc1cccc2cc(O)cc(-c3ncc4c(N5CC6CCC(C5)N6)nc(N5CC(CN6CCC(CC7CN(c8cc([C@@H](C(=O)N9C[C@H](O)C[C@H]9C(=O)N[C@@H](C)c9ccc(-c%10scnc%10C)cc9)C(C)C)on8)C7)CC6)C5)nc4c3F)c12]

The title compound was made in an analogous manner to (2S,4R)-1-[(2R)-2-(3-{3-[(1-{[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) azetidin-3-yl]methyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide, starting from tert-butyl 3-[7-(8-ethyl-3-hydroxy-1-naphthyl)-8-fluoro-2-(3-formylazetidin-1-yl)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide. (white solid). MS (ESI) m/z: 1131.7 [M+1]⁺; 1H NMR (400 MHz, CD₃OD) δ 8.91-8.89 (m, 1H), 8.84 (s, 1H), 8.44-8.40 (m, 2H), 7.65 (d, J=8.0 Hz, 1H), 7.46-7.37 (m, 5H), 7.30 (d, J=2.4 Hz, 1H), 7.18 (d, J=6.8 Hz, 1H), 7.00 (d, J=2.4 Hz, 1H), 5.89 (s, 1H), 5.01 (d, J=7.0 Hz, 1H), 4.68-4.58 (m, 5H), 4.49-4.44 (m, 3H), 4.11-4.04 (m, 6H), 3.78-3.57 (m, 7H), 3.25-3.18 (m, 1H), 2.89-2.74 (m, 3H), 2.51-2.50 (m, 3H), 2.42-2.29 (m, 4H), 2.10 (s, 4H), 1.99-1.87 (m, 3H), 1.69-1.50 (m, 8H), 1.07 (d, J=6.4 Hz, 3H), 0.95-0.91 (m, 6H).

Exemplary Synthesis of (2S,4R)-1-[(2R)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]piperazin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 191)

Step 1: Preparation of tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate

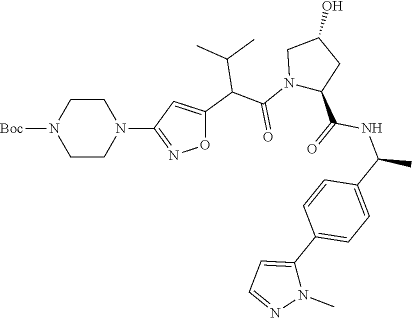 [SMILES: CC(C)C(C(=O)N1C[C@H](O)C[C@H]1C(=O)N[C@@H](C)c1ccc(-c2ccnn2C)cc1)c1cc(N2CCN(C(=O)OC(C)(C)C)CC2)no1]

To a solution of 2-[3-(4-tert-butoxycarbonylpiperazin-1-yl)isoxazol-5-yl]-3-methyl-butanoic acid (5.0 g, 14 mmol) in DMF (50 mL) were added DIEA (19.7 mL, 113 mmol), (2S,4R)-4-hydroxy-N-[(1S)-1-[4-(2-methylpyrazol-3-yl)phenyl]ethyl]pyrrolidine-2-carboxamide hydrochloride (5.5 g, 15 mmol) and HATU (5.9 g, 15 mmol). The mixture was stirred at 25° C. for 30 min, diluted with water (100 mL) and extracted with ethyl acetate (100 mL×3). The combined extracts were washed with brine (100 mL×2), dried over anhydrous Na₂SO₄, filtered, and concentrated under reduced pressure. The residue was purified by column chromatography (0-10% MeOH in CH₂Cl₂) to afford tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate (5.8 g, 63%) as a yellow solid. MS (ESI) m/z: 650.2 [M+H]⁺.

Step 2: Preparation of tert-butyl 4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate & tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate

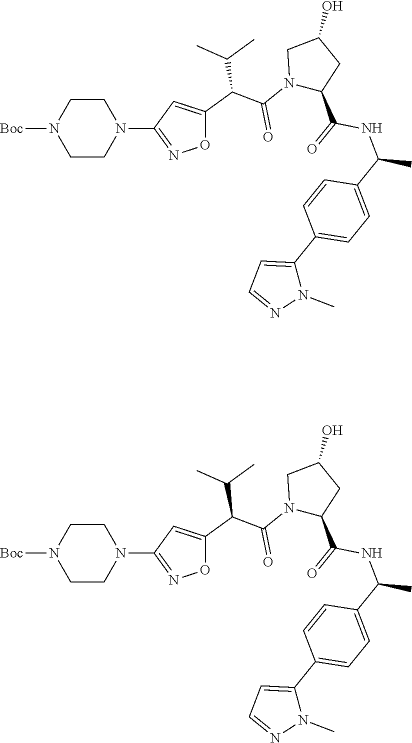 [SMILES: CC(C)[C@H](C(=O)N1C[C@H](O)C[C@H]1C(=O)N[C@@H](C)c1ccc(-c2ccnn2C)cc1)c1cc(N2CCN(C(=O)OC(C)(C)C)CC2)no1 ; CC(C)[C@@H](C(=O)N1C[C@H](O)C[C@H]1C(=O)N[C@@H](C)c1ccc(-c2ccnn2C)cc1)c1cc(N2CCN(C(=O)OC(C)(C)C)CC2)no1]

Racemic tert-butyl 4-[[5-[1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate (5.8 g) was separated by chiral SFC (column: Daicel ChiralPak IG (250*30 mm, 10 um); mobile phase: [0.1% NH₃H₂O EtOH]; B %: 40%-40%, Gradient time: 60 min). Fraction 1 was obtained as tert-butyl 4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate (2.8 g) as a yellow solid. MS (ESI) m/z: 650.3 [M+H]⁺. Fraction 2 was obtained as tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate (2.1 g) as a yellow solid. MS (ESI) m/z: 650.3 [M+H]⁺.

Step 3: Preparation of (2S,4R)-4-hydroxy-1-[(2R)-3-methyl-2-(3-piperazin-1-ylisoxazol-5-yl)butanoyl]-N-[(1S)-1-[4-(2-methylpyrazol-3-yl)phenyl]ethyl]pyrrolidine-2-carboxamide

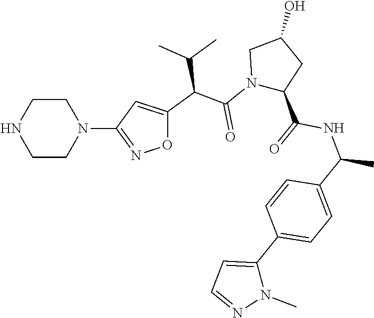 [SMILES: CC(C)[C@@H](C(=O)N1C[C@H](O)C[C@H]1C(=O)N[C@@H](C)c1ccc(-c2ccnn2C)cc1)c1cc(N2CCNCC2)no1]

A solution of tert-butyl 4-[[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate (2.1 g, 3.2 mmol) in CH₂Cl₂ (15 mL) and 4 M HCl in dioxane (15 mL) was stirred at 25° C. for 30 min. The mixture was concentrated at 25° C. The residue was basified with saturated Na₂CO₃ solution until pH 8 and extracted with CH₂Cl₂/MeOH (20 mL×5, v/v=10/1). The combined organic layers were washed with brine (15 mL×3), dried over anhydrous Na₂SO₄, filtered, and concentrated under reduced pressure to afford (2S,4R)-4-hydroxy-1-[(2R)-3-methyl-2-(3-piperazin-1-ylisoxazol-5-yl)butanoyl]-N-[(1S)-1-[4-(2-methylpyrazol-3-yl)phenyl]ethyl]pyrrolidine-2-carboxamide (1.9 g) as a yellow solid, which was used directly in the next step. MS (ESI) m/z: 550.1 [M+H]⁺.

Step 4: Preparation of tert-butyl 4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]piperidine-1-carboxylate

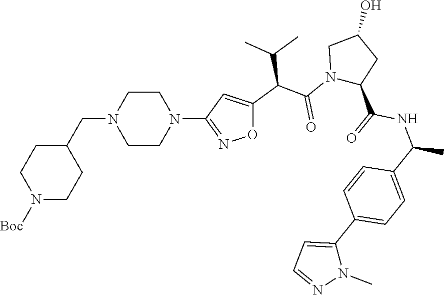 [SMILES: CC(C)[C@@H](C(=O)N1C[C@H](O)C[C@H]1C(=O)N[C@@H](C)c1ccc(-c2ccnn2C)cc1)c1cc(N2CCN(CC3CCN(C(=O)OC(C)(C)C)CC3)CC2)no1]

To a solution of (2S,4R)-4-hydroxy-1-[(2R)-3-methyl-2-(3-piperazin-1-ylisoxazol-5-yl)butanoyl]-N-[(1S)-1-[4-(2-methylpyrazol-3-yl)phenyl]ethyl]pyrrolidine-2-carboxamide (900 mg, 1.6 mmol) and tert-butyl 4-formylpiperidine-1-carboxylate (873 mg, 4.1 mmol) in CH₂Cl₂ (7 mL) and i-PrOH (7 mL), then acetic acid (0.5 mL, 8 mmol) and 2-methylpyridine borane (700 mg, 6 mmol) were added. The mixture was stirred at 25° C. for 1 h, then concentrated in vacuum. The residue was purified by column chromatography (0-3% methanol (1N NH₃ as additive) in dichloromethane) to afford tert-butyl 4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]piperidine-1-carboxylate (1.16 g, 89%) as a yellow solid. MS (ESI) m/z: 747.2 [M+H]⁺.

Step 5: Preparation of (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(2-methylpyrazol-3-yl)phenyl]ethyl]pyrrolidine-2-carboxamide

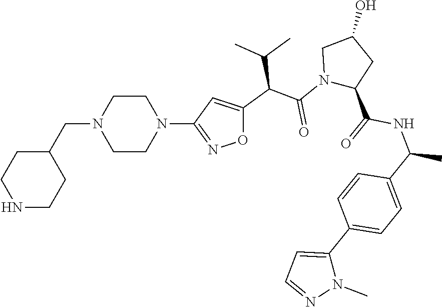 [SMILES: CC(C)[C@@H](C(=O)N1C[C@H](O)C[C@H]1C(=O)N[C@@H](C)c1ccc(-c2ccnn2C)cc1)c1cc(N2CCN(CC3CCNCC3)CC2)no1]

To a solution of tert-butyl 4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]piperidine-1-carboxylate (1.16 g, 1.5 mmol) in CH₂Cl₂ (10 mL) was added 4 M HCl in dioxane (4.0 mL). The mixture was stirred at 25° C. for 1 h. The reaction mixture was azeotroped with petroleum ether (30 mL×2) and concentrated. The residue was diluted with aqueous Na₂CO₃ (10 mL) to adjust the pH to 10, diluted with water (15 mL) and extracted with dichloromethane (20 mL×3). The combined organic layers were washed brine (20 mL), dried over Na₂SO₄, filtered and concentrated to afford (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(2-methylpyrazol-3-yl)phenyl]ethyl]pyrrolidine-2-carboxamide (763 mg, 67%) as a yellow foam. MS (ESI) m/z: 647.3 [M+H]⁺.

Step 6: Preparation of tert-butyl 3-[7-(8-ethyl-3-hydroxy-1-naphthyl)-8-fluoro-2-[2-[[4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate

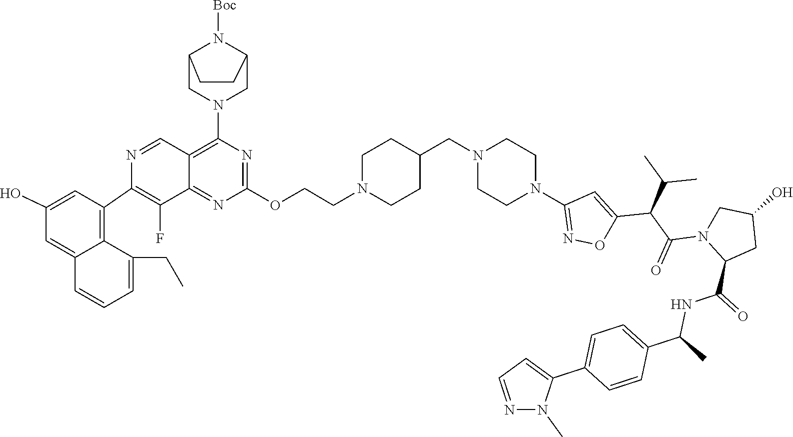 [SMILES: CCc1cccc2cc(O)cc(-c3ncc4c(N5CC6CCC(C5)N6C(=O)OC(C)(C)C)nc(OCCN5CCC(CN6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (230 mg, 0.4 mmol) and (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(2-methylpyrazol-3-yl)phenyl]ethyl]pyrrolidine-2-carboxamide (253 mg, 0.4 mmol) in CH₂Cl₂ (5 mL) and i-PrOH (5 mL) were added borane; 2-methylpyridine (167 mg, 1.6 mmol) and AcOH (0.12 mL, 2 mmol). The mixture was stirred at 25° C. for 1 h, then concentrated in vacuum. The residue was purified by column chromatography (0-10% methanol in dichloromethane) to afford tert-butyl 3-[7-(8-ethyl-3-hydroxy-1-naphthyl)-8-fluoro-2-[2-[[4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (350 mg, 69%) as a yellow foam. MS (ESI) m/z: 1219.8 [M+H]⁺.

Step 7: Preparation of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

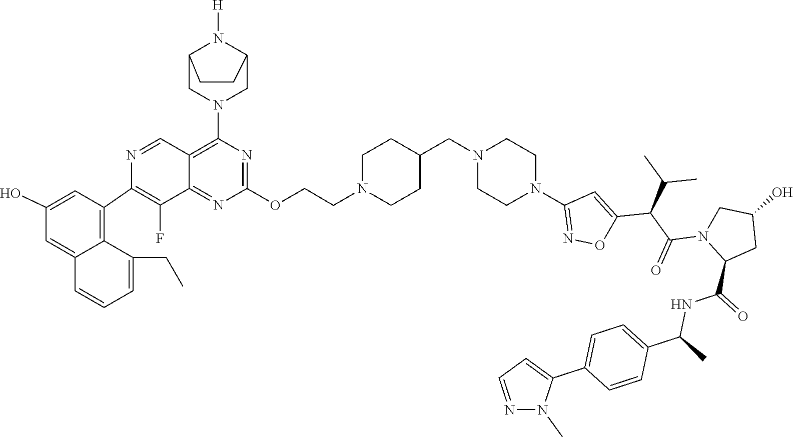 [SMILES: CCc1cccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12]

To a solution of tert-butyl 3-[7-(8-ethyl-3-hydroxy-1-naphthyl)-8-fluoro-2-[2-[[4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (350 mg, 0.27 mmol) in CH₂Cl₂ (3 mL) was added 4 M HCl in dioxane (3.0 mL). The mixture was stirred at 25° C. for 1 h. The reaction mixture was azeotroped with petroleum ether (20 mL×2) and concentrated. The pH of the residue was adjusted to 8 by progressively adding aqueous NaHCO₃. The mixture was extracted with dichloromethane:methanol (10:1=v:v, 55 mL). The combined organic layers were washed brine (10 mL), dried over Na₂SO₄, filtered, and concentrated under reduced pressure to give the crude product, which was purified by prep-HPLC [Column: Xtimate C18 150*40 mm*10 um; Eluent: gradient 0%-30% acetonitrile in water (FA); Gradient time: 36 min; Hold time: 3 min; Flow rate: 60 mL/min] to afford (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (163.2 mg, 52%, di-formic acid salt) as a yellow solid. MS (ESI) m/z: 1118.7 [M+H]⁺; ¹H NMR (400 MHz, CD₃OD) δ 9.11 (s, 1H), 8.45 (s, 2H), 7.64 (d, J=8.0 Hz, 1H), 7.49 (d, J=2.0 Hz, 1H), 7.45 (s, 3H), 7.38 (dd, J=8.2, 15.6 Hz, 2H), 7.30 (d, J=2.4 Hz, 1H), 7.17 (d, J=7.2 Hz, 1H), 7.01 (d, J=2.8 Hz, 1H), 6.34 (d, J=2.0 Hz, 1H), 6.10 (s, 1H), 5.08-5.01 (m, 1H), 4.82 (d, J=4.4 Hz, 3H), 4.78 (s, 1H), 4.76-4.70 (m, 2H), 4.51 (t, J=8.4 Hz, 1H), 4.44 (s, 1H), 4.01-3.96 (m, 2H), 3.89 (s, 1H), 3.85 (s, 3H), 3.66-3.57 (m, 4H), 3.45 (d, J=4.8 Hz, 2H), 3.24 (s, 4H), 2.90 (t, J=11.2 Hz, 2H), 2.54 (d, J=4.4 Hz, 4H), 2.39-2.32 (m, 2H), 2.30-2.26 (m, 3H), 2.05-1.99 (m, 5H), 1.98-1.90 (m, 3H), 1.59 (d, J=6.8 Hz, 1H), 1.52 (d, J=7.2 Hz, 3H), 1.48-1.39 (m, 2H), 1.05 (d, J=6.8 Hz, 3H), 0.92-0.88 (m, 6H).

Exemplary Synthesis of (2S,4R)-1-[(2R)-2-(3-{3-[(1-{[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) piperidin-4-yl]methyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 192)

Step 1: Preparation of tert-butyl 3-[2-[[4-(dimethoxymethyl)-1-piperidyl]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

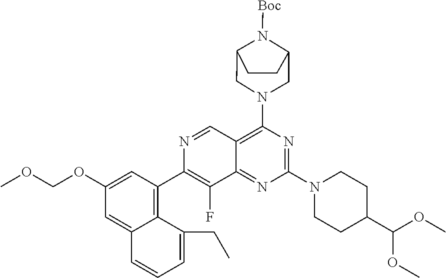

To a solution of tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (500 mg, 0.8 mmol) in tetrahydrofuran (10 mL) were added DIEA (535 uL, 3 mmol) and 4-(dimethoxymethyl)piperidine (244 mg, 1.5 mmol). The mixture was stirred at 25° C. for 3 h, then concentrated. The residue was purified by flash column chromatography (0-28% ethyl acetate in petroleum ether) to afford tert-butyl 3-[2-[[4-(dimethoxymethyl)-1-piperidyl]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (520 mg, 90%) as a white solid. MS (ESI) m/z: 731.7 [M+1]⁺.

Step 2: Preparation of tert-butyl 3-[7-(3-tert-butoxycarbonyloxy-8-ethyl-1-naphthyl)-8-fluoro-2-(4-formyl-1-piperidyl)pyrido[4,3-d]pyrimidin-4-yl]-3,8-diazabicyclo[3.2.1]octane-8-carboxylate

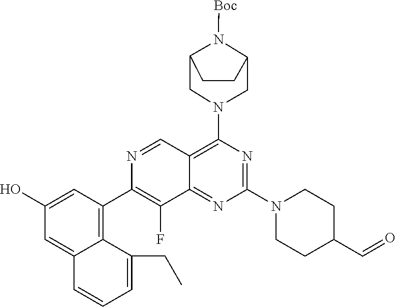 [SMILES: CCc1cccc2cc(O)cc(-c3ncc4c(N5CC6CCC(C5)N6C(=O)OC(C)(C)C)nc(N5CCC(C=O)CC5)nc4c3F)c12]

To a solution of tert-butyl 3-[2-[[4-(dimethoxymethyl)-1-piperidyl]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (500 mg, 0.7 mmol, 1.0 eq) in acetone (1.5 mL) was added HCl (1.5 mL, 12 M), and the mixture was stirred at 20° C. for 10 min. Then a solution of Boc₂O (314 uL, 1.4 mmol, 2.0 eq) in H₂O (3 mL) and tetrahydrofuran (3 mL) was added, followed by the addition of NaHCO₃ (2.0 g, 23.94 mmol, 35 eq). The mixture was stirred at 20° C. for 1 h, diluted with water (10 mL) and extracted with ethyl acetate (20 mL×3). The combined organic phase was washed with brine (10 mL), dried over anhydrous sodium sulfate, filtered, and concentrated over vacuum. The residue was purified by column chromatography (0-37% ethyl acetate in petroleum ether) to afford tert-butyl 3-[7-(3-tert-butoxycarbonyloxy-8-ethyl-1-naphthyl)-8-fluoro-2-(4-formyl-1-piperidyl)pyrido[4,3-d]pyrimidin-4-yl]-3,8-diazabicyclo[3.2.1]octane-8-carboxylate (310 mg, 54%) as a yellow solid. MS (ESI) m/z: 741.4 [M+1]⁺.

Step 3: Preparation of tert-butyl 3-[7-(8-ethyl-3-hydroxy-1-naphthyl)-8-fluoro-2-[[4-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]methyl]-1-piperidyl]pyrido[4,3-d]pyrimidin-4-yl]-3,8-diazabicyclo[3.2.1]octane-8-carboxylate

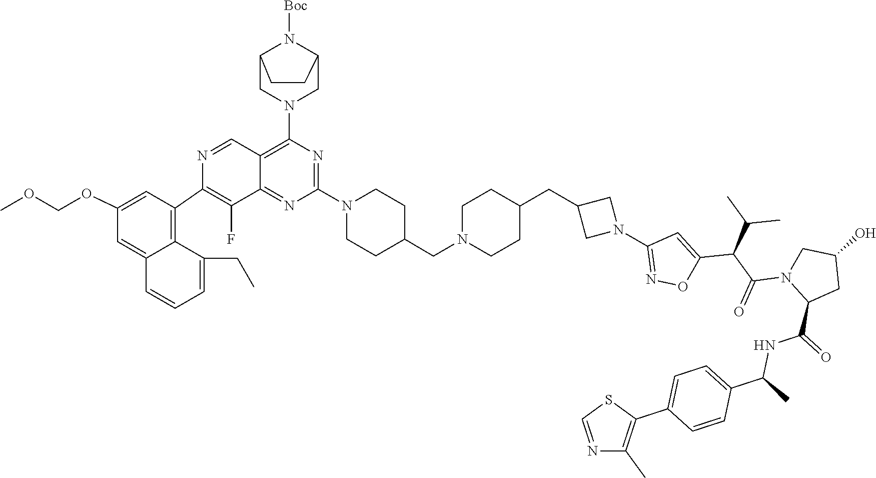 [SMILES: CCc1cccc2cc(OCOC)cc(-c3ncc4c(N5CC6CCC(C5)N6C(=O)OC(C)(C)C)nc(N5CCC(CN6CCC(CC7CN(c8cc([C@H](C(=O)N9C[C@H](O)C[C@H]9C(=O)N[C@@H](C)c9ccc(-c%10scnc%10C)cc9)C(C)C)on8)C7)CC6)CC5)nc4c3F)c12]

To a solution of tert-butyl 3-[7-(8-ethyl-3-hydroxy-1-naphthyl)-8-fluoro-2-(4-formyl-1-piperidyl)pyrido[4,3-d]pyrimidin-4-yl]-3,8-diazabicyclo[3.2.1]octane-8-carboxylate (230 mg, 0.4 mmol) and (2S,4R)-4-hydroxy-1-[(2R)-3-methyl-2-[3-[[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide (228 mg, 0.4 mmol) in i-PrOH (2 mL) and CH₂Cl₂ (4 mL) were added AcOH (103 uL, 1.8 mmol) and 2-methylpyridine borane (192 mg, 1.8 mmol). The mixture was stirred at 25° C. for 1 h. The reaction mixture was concentrated, the residue was purified by flash column (0-7% methanol in dichloromethane) to afford tert-butyl 3-[7-(8-ethyl-3-hydroxy-1-naphthyl)-8-fluoro-2-[[4-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]methyl]-1-piperidyl]pyrido[4,3-d]pyrimidin-4-yl]-3,8-diazabicyclo[3.2.1]octane-8-carboxylate (300 mg, 66%) as yellow solid. MS (ESI) m/z: 1259.6 [M+1]⁺.

Step 4: Preparation of (2S,4R)-1-[(2R)-2-[3-[[3-[[1-[1-[4-(3,8-diazabicyclo[3.2.1]octan-3-yl)-7-(8-ethyl-3-hydroxy-1-naphthyl)-8-fluoro-pyrido[4,3-d]pyrimidin-2-yl]-4-piperidyl]methyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

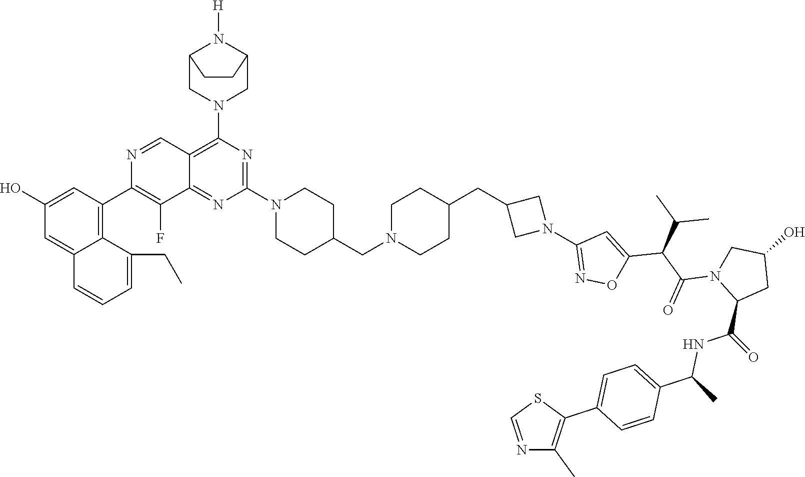 [SMILES: CCc1cccc2cc(O)cc(-c3ncc4c(N5CC6CCC(C5)N6)nc(N5CCC(CN6CCC(CC7CN(c8cc([C@H](C(=O)N9C[C@H](O)C[C@H]9C(=O)N[C@@H](C)c9ccc(-c%10scnc%10C)cc9)C(C)C)on8)C7)CC6)CC5)nc4c3F)c12]

To a solution of tert-butyl 3-[7-(8-ethyl-3-hydroxy-1-naphthyl)-8-fluoro-2-[[4-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-1-piperidyl]methyl]-1-piperidyl]pyrido[4,3-d]pyrimidin-4-yl]-3,8-diazabicyclo[3.2.1]octane-8-carboxylate (300 mg, 0.2 mmol, 1.0 eq) in CH₂Cl₂ (6 mL) was added TFA (1.4 mL, 19 mmol). The mixture was stirred at 25° C. for 30 min, then concentrated in vacuum. The pH of the residue was adjusted with saturated aqueous NaHCO₃ to 8 and extracted with CH₂Cl₂/MeOH (20 mL×4, v:v=10:1). The combined organic phase was washed with brine (10 mL), dried over anhydrous sodium sulfate, filtered and concentrated vacuum. The residue was purified by prep-HPLC (column: Xtimate C18 over 150*40 mm*10 um; mobile phase: [water (formic acid)-acetonitrile]; B %: 0%-38%, 36 min) to afford (2S,4R)-1-[(2R)-2-[3-[[3-[[1-[[1-[4-(3,8-diazabicyclo[3.2.1]octan-3-yl)-7-(8-ethyl-3-hydroxy-1-naphthyl)-8-fluoro-pyrido[4,3-d]pyrimidin-2-yl]-4-piperidyl]methyl]-4-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (151.6 mg, formic acid salt, 52%) as a white solid. MS (ESI) m/z: 1159.7 [M+1]⁺; 1H NMR (400 MHz, CD₃OD) δ 8.88 (s, 1H), 8.77 (s, 1H), 7.62 (d, J=7.6 Hz, 1H), 7.47-7.32 (m, 5H), 7.28 (d, J=2.4 Hz, 1H), 7.16 (d, J=6.8 Hz, 1H), 6.99 (d, J=2.4 Hz, 1H), 5.84 (s, 1H), 5.04-5.02 (m, 1H), 4.62 (s, 4H), 4.56-4.48 (m, 3H), 4.44 (s, 1H), 4.09-4.04 (m, 2H), 3.93 (s, 2H), 3.85-3.82 (m, 1H), 3.73-3.56 (m, 6H), 3.49-3.42 (m, 2H), 3.08-3.02 (m, 2H), 2.91-2.87 (m, 2H), 2.76-2.74 (m, 2H), 2.48 (s, 3H), 2.41-2.30 (m, 3H), 2.21-2.16 (m, 2H), 2.01 (s, 4H), 1.94-1.85 (m, 4H), 1.69 (t, J=6.8 Hz, 2H), 1.59 (d, J=7.2 Hz, 1H), 1.54-1.47 (m, 4H), 1.37-1.24 (m, 3H), 1.05 (d, J=6.4 Hz, 3H), 0.94-0.86 (m, 6H).

Exemplary Synthesis of (2S,4R)-1-[(2S)-2-(3-{3-[(1-{[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) piperidin-4-yl]methyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 193)

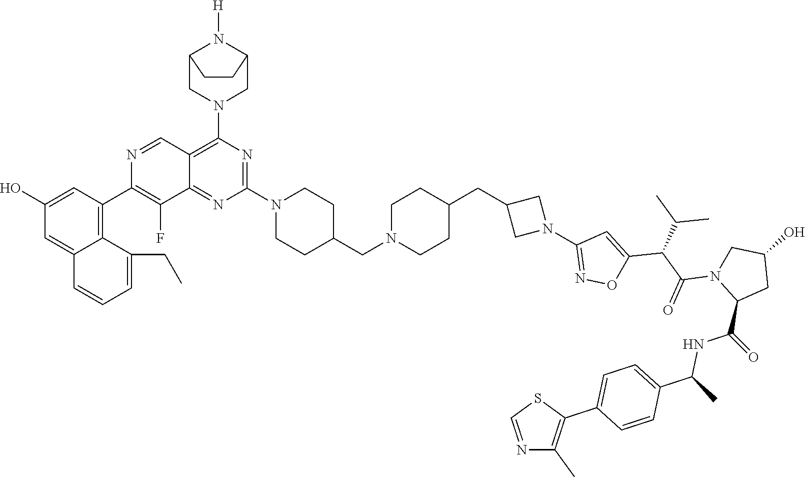 [SMILES: CCc1cccc2cc(O)cc(-c3ncc4c(N5CC6CCC(C5)N6)nc(N5CCC(CN6CCC(CC7CN(c8cc([C@@H](C(=O)N9C[C@H](O)C[C@H]9C(=O)N[C@@H](C)c9ccc(-c%10scnc%10C)cc9)C(C)C)on8)C7)CC6)CC5)nc4c3F)c12]

The title compound was prepared analogously to (2S,4R)-1-[(2R)-2-(3-{3-[(1-{[1-(4-{3,8-diazabicyclo[3.2.1]octan-3-yl}-7-(8-ethyl-3-hydroxynaphthalen-1-yl)-8-fluoropyrido[4,3-d]pyrimidin-2-yl) piperidin-4-yl]methyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide, starting from tert-butyl 3-[7-(3-tert-butoxycarbonyloxy-8-ethyl-1-naphthyl)-8-fluoro-2-(4-formyl-1-piperidyl)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). MS (ESI) m/z: 1159.7 [M+1]⁺; 1H NMR (400 MHz, CD₃OD) δ 8.88 (s, 1H), 8.78 (s, 1H), 7.62 (d, J=7.6 Hz, 1H), 7.49-7.33 (m, 5H), 7.28 (d, J=2.4 Hz, 1H), 7.16 (d, J=6.8 Hz, 1H), 6.99 (d, J=2.4 Hz, 1H), 5.86 (s, 1H), 5.02-4.98 (m, 1H), 4.74-4.49 (m, 7H), 4.43 (s, 1H), 4.07-3.96 (m, 4H), 3.75-3.64 (m, 5H), 3.59-3.53 (m, 2H), 3.50-3.44 (m, 2H), 3.06 (t, J=12.4 Hz, 2H), 2.91-2.90 (m, 2H), 2.87-2.79 (m, 2H), 2.48 (s, 3H), 2.40-2.31 (m, 3H), 2.22-2.20 (m, 2H), 2.03 (s, 3H), 1.96-1.85 (m, 5H), 1.66-1.63 (m, 2H), 1.51 (d, J=7.2 Hz, 5H), 1.37-1.25 (m, 3H), 1.05 (d, J=6.4 Hz, 3H), 0.95-0.89 (m, 6H).

Exemplary Synthesis of (2S,4R)-1-[(2R)-2-{3-[(2R)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-2-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 194)

Step 1: Preparation of tert-butyl 4-fluoro-4-[[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]piperidine-1-carboxylate

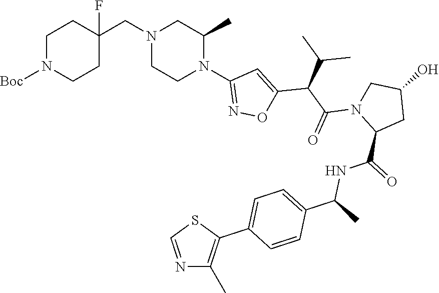 [SMILES: Cc1ncsc1-c1ccc([C@H](C)NC(=O)[C@@H]2C[C@@H](O)CN2C(=O)[C@@H](c2cc(N3CCN(CC4(F)CCN(C(=O)OC(C)(C)C)CC4)C[C@H]3C)no2)C(C)C)cc1]

To a solution of (2S,4R)-4-hydroxy-1-[(2R)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide (240 mg, 0.4 mmol, 1.0 eq) and tert-butyl 4-fluoro-4-formyl-piperidine-1-carboxylate (478 mg, 2 mmol, 5.0 eq) in CH₂Cl₂ (1 mL) and i-PrOH (1 mL) was added AcOH (118 uL, 2 mmol, 5.0 eq) and 2-methylpyridine borane (221 mg, 2 mmol, 5.0 eq). The mixture was stirred at 25° C. for 10 h. Triethylamine was added until pH 8 and concentrated under reduced pressure. The residue was purified by column chromatography (0-5% MeOH in CH₂Cl₂) to afford tert-butyl 4-fluoro-4-[[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]piperidine-1-carboxylate (280 mg, 85%) as a yellow solid. MS (ESI) m/z: 796.2 [M+H]⁺

Step 2: Preparation of (2S,4R)-1-[(2R)-2-[3-[(2R)-4-[(4-fluoro-4-piperidyl)methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

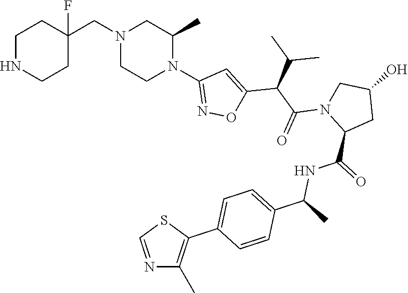 [SMILES: Cc1ncsc1-c1ccc([C@H](C)NC(=O)[C@@H]2C[C@@H](O)CN2C(=O)[C@@H](c2cc(N3CCN(CC4(F)CCNCC4)C[C@H]3C)no2)C(C)C)cc1]

To a solution of tert-butyl 4-fluoro-4-[[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]piperidine-1-carboxylate (280 mg, 0.3 mmol, 1.0 eq) in CH₂Cl₂ (1 mL) was added 4 M HCl in dioxane (4 mL). The mixture was stirred at 0° C. for 25 min, then concentrated at 25° C. The mixture was basified with saturated NaHCO₃ solution until pH 8 and extracted with CH₂Cl₂/MeOH (20 mL×3, v/v=10/1). The combined extracts were washed with brine (15 mL×3), dried over anhydrous Na₂SO₄, filtered and concentrated under reduced pressure to afford (2S,4R)-1-[(2R)-2-[3-[(2R)-4-[(4-fluoro-4-piperidyl)methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (210 mg) as a yellow solid, which was used directly in the next step. MS (ESI) m/z: 696.3 [M+H]⁺.

Step 3: Preparation of tert-butyl 3-[7-(8-ethyl-3-hydroxy-1-naphthyl)-8-fluoro-2-[2-[[4-fluoro-4-[[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]-1-piperidyl]ethoxy]pyrido[4,3-d]pyrimidin-4-yl]-3,8-diazabicyclo[3.2.1]octane-8-carboxylate

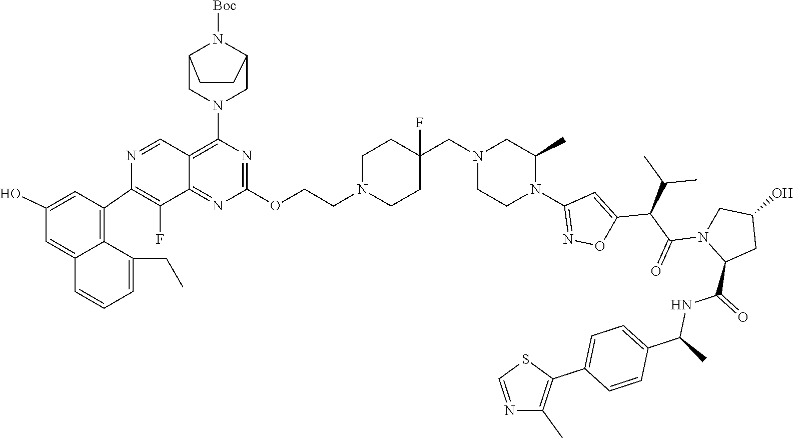 [SMILES: CCc1cccc2cc(O)cc(-c3ncc4c(N5CC6CCC(C5)N6C(=O)OC(C)(C)C)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)[C@H](C)C6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (213 mg, 0.3 mmol, 1.0 eq) and (2S,4R)-1-[(2R)-2-[3-[(2R)-4-[(4-fluoro-4-piperidyl)methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (210 mg, 0.3 mmol, 1.0 eq) in CH₂Cl₂ (3 mL) and i-PrOH (3 mL) were added AcOH (86 uL, 1.5 mmol, 5.0 eq) and 2-methylpyridine borane (161 mg, 1.5 mmol, 5.0 eq). The mixture was stirred at 25° C. for 2 h. Trimethylamine was added until pH 8 and then concentrated under reduced pressure. The residue was purified by column chromatography (0-5% MeOH in CH₂Cl₂) to afford tert-butyl 3-[7-(8-ethyl-3-hydroxy-1-naphthyl)-8-fluoro-2-[2-[[4-fluoro-4-[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]-1-piperidyl]ethoxy]pyrido[4,3-d]pyrimidin-4-yl]-3,8-diazabicyclo[3.2.1]octane-8-carboxylate (360 mg, 80%) as a yellow solid. MS (ESI) m/z: 1289.7 [M+Na]⁺.

Step 4: Preparation of (2S,4R)-1-[(2R)-2-[3-[(2R)-4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

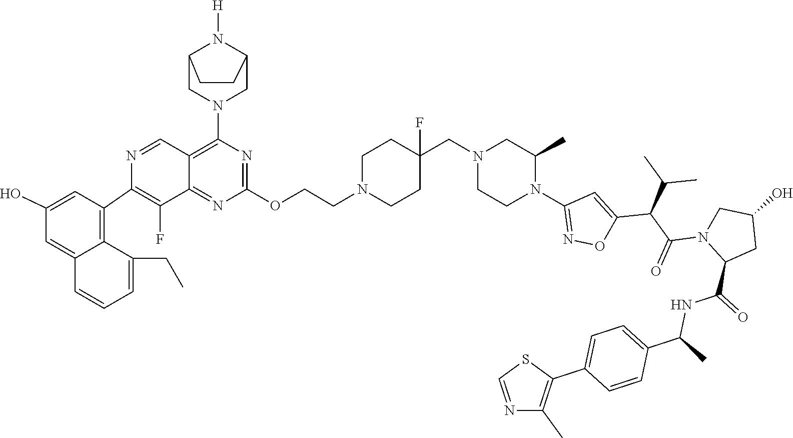 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)[C@H](C)C6)CC5)nc4c3F)c12]

A solution of tert-butyl 3-[7-(8-ethyl-3-hydroxy-1-naphthyl)-8-fluoro-2-[2-[[4-fluoro-4-[[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]-1-piperidyl]ethoxy]pyrido[4,3-d]pyrimidin-4-yl]-3,8-diazabicyclo[3.2.1]octane-8-carboxylate (360 mg, 0.2 mmol, 1.0 eq) in CH₂Cl₂ (3 mL) and 4 M HCl in dioxane (1 mL) was stirred at 25° C. for 60 min. The reaction mixture was suspended in petroleum ether (10 mL) and filtered, concentrated under reduced pressure. The residue was suspended in THF (5 mL) and triethylamine (0.3 mL) and filtered. The filtrate was concentrated reduced pressure and purified by prep-HPLC (column: Xtimate C18 150*40 mm*10 um; mobile phase: [water (FA)-ACN]; B %: 0%-38%, 36 min) to afford (2S,4R)-1-[(2R)-2-[3-[(2R)-4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (150.2 mg, 44%) as a white solid. MS (ESI) m/z: 1167.7 [M+H]⁺; ¹H NMR (400 MHz, CD₃OD) δ 9.07 (s, 1H), 8.87 (s, 1H), 7.65 (d, J=8.4 Hz, 1H), 7.47-7.32 (m, 5H), 7.29 (t, J=2.0 Hz, 1H), 7.16 (d, J=7.2 Hz, 1H), 7.01 (d, J=2.4 Hz, 1H), 6.06 (s, 1H), 5.02-4.96 (m, 1H), 4.69-4.56 (m, 8H), 4.53 (m, 1H), 4.43 (s, 1H), 3.77-3.66 (m, 8H), 2.95-2.85 (m, 5H), 2.57-2.50 (m, 2H), 2.49 (s, 1H), 2.47 (s, 3H), 2.40-2.21 (m, 6H), 2.02-1.75 (m, 9H), 1.48 (d, J=7.2 Hz, 3H), 1.19 (d, J=6.4 Hz, 3H), 1.04 (d, J=6.4 Hz, 3H), 0.93-0.86 (m, 6H).

Exemplary Synthesis of (2S,4R)-1-[(2S)-2-{3-[(3R)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-3-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 195)

Step 1: Preparation of tert-butyl 4-fluoro-4-[[(2R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]piperidine-1-carboxylate

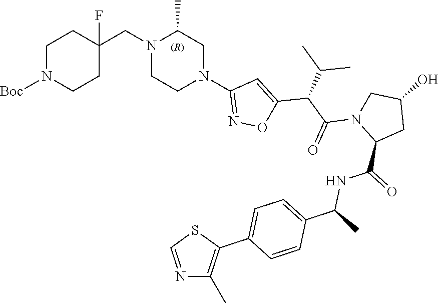

To a reaction mixture of tert-butyl 4-fluoro-4-formyl-piperidine-1-carboxylate (896 mg, 3.9 mmol, 5.0 eq) and (2S,4R)-4-hydroxy-1-[(2S)-3-methyl-2-[3-[(3R)-3-methylpiperazin-1-yl]isoxazol-5-yl]butanoyl]-N-[(1S)-1-[4-(4-methylthiazol-5-yl)phenyl]ethyl]pyrrolidine-2-carboxamide (450 mg, 0.8 mmol, 1.0 eq) in DCE (15 mL) and i-PrOH (3 mL) was added HOAc (178 uL, 3 mmol, 4.0 eq) and 2-methylpyridine borane (414 mg, 3.9 mmol, 5.0 eq), the resulting suspension was stirred at 50° C. for 63 h. The reaction mixture was concentrated under reduced pressure. The residue was purified by column chromatography (0-4% MeOH in CH₂Cl₂) to afford tert-butyl 4-fluoro-4-[[(2R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]piperidine-1-carboxylate (324 mg, 49%) as a white solid. MS (ESI) m/z: 796.2 [M+H]⁺.

Step 2: Preparation of (2S,4R)-1-[(2S)-2-[3-[(3R)-4-[(4-fluoro-4-piperidyl)methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

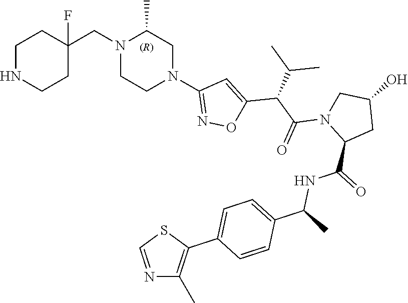

To a reaction mixture of tert-butyl 4-fluoro-4-[[(2R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]piperidine-1-carboxylate (324 mg, 93% purity, 0.4 mmol, 1.0 eq) in CH₂Cl₂ (3 mL) was added 4 M HCl in dioxane (3 mL), the resulting suspension was stirred at 25° C. for 15 min. The reaction mixture was suspended in petroleum ether (30 mL), the precipitate was separated, dissolved in THF (30 mL), adjusted with TEA to pH 8. The mixture was filtered and concentrated under reduced pressure to afford (2S,4R)-1-[(2S)-2-[3-[(3R)-4-[(4-fluoro-4-piperidyl)methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (263 mg, crude) as a white solid, which was used in the next step directly. MS (ESI) m/z: 696.4 [M+H]⁺.

Step 3: Preparation of tert-butyl 3-[7-(8-ethyl-3-hydroxy-1-naphthyl)-8-fluoro-2-[2-[[4-fluoro-4-[[(2R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]-1-piperidyl]ethoxy]pyrido[4,3-d]pyrimidin-4-yl]-3,8-diazabicyclo[3.2.1]octane-8-carboxylate

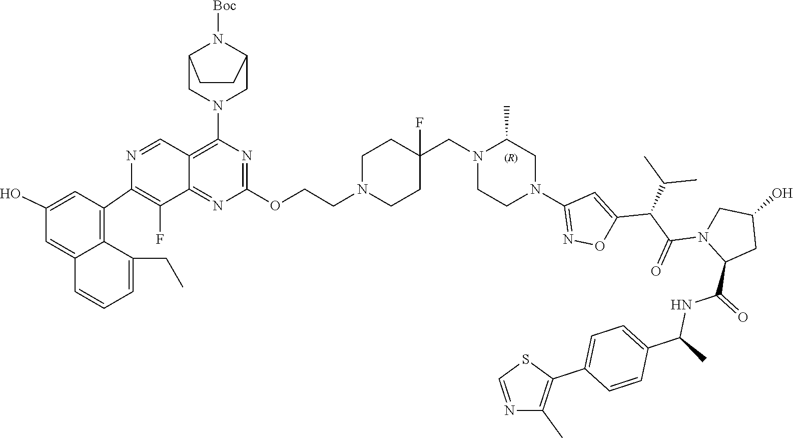

To a reaction mixture of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (249 mg, 0.4 mmol, 1.0 eq) and (2S,4R)-1-[(2S)-2-[3-[(3R)-4-[(4-fluoro-4-piperidyl)methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (263 mg, 0.4 mmol, 1.0 eq) in CH₂Cl₂ (6 mL) and i-PrOH (1 mL) was added HOAc (86 uL, 1.5 mmol, 4.0 eq) and 2-methylpyridine borane (202 mg, 1.9 mmol, 5.0 eq), the resulting suspension was stirred at 25° C. for 15 h. The reaction mixture was concentrated under reduced pressure, the residue was purified by column chromatography (0-6% then up to 8% MeOH in CH₂Cl₂) to afford tert-butyl 3-[7-(8-ethyl-3-hydroxy-1-naphthyl)-8-fluoro-2-[2-[[4-fluoro-4-[[(2R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]-1-piperidyl]ethoxy]pyrido[4,3-d]pyrimidin-4-yl]-3,8-diazabicyclo[3.2.1]octane-8-carboxylate (395 mg, 75%) as a yellow solid. MS (ESI) m/z: 1267.3 [M+H]⁺.

Step 4: Preparation of (2S,4R)-1-[(2S)-2-[3-[(3R)-4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

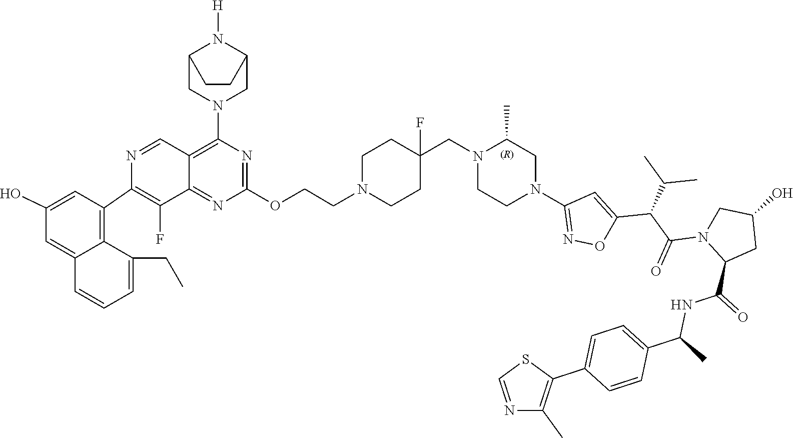

To a reaction mixture of tert-butyl 3-[7-(8-ethyl-3-hydroxy-1-naphthyl)-8-fluoro-2-[2-[[4-fluoro-4-[[(2R)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]-1-piperidyl]ethoxy]pyrido[4,3-d]pyrimidin-4-yl]-3,8-diazabicyclo[3.2.1]octane-8-carboxylate (395 mg, 0.3 mmol, 1.0 eq) in CH₂Cl₂ (4 mL) was added 4 M HCl in dioxane (4 mL), the resulting suspension was stirred at 25° C. for 0.25 h. The reaction mixture was suspended in petroleum ether (30 mL), the solid was separated, dissolved in THF (30 mL), adjusted the pH with triethylamine to 8. The mixture was filtered and concentrated under reduced pressure. The crude product was purified by prep-HPLC (column: Xtimate C18 150*40 mm*10 um; mobile phase: [water (NH₄HCO₃)-ACN]; Begin B: 32; End B: 72; Gradient Time (min): 36; 100% B Hold Time (min): 3; Flow Rate (ml/min): 60) to afford (2S,4R)-1-[(2S)-2-[3-[(3R)-4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (153.1 mg, 46%) as a white solid. MS (ESI) m/z: 1167.8 [M+H]⁺; ¹H NMR (400 MHz, DMSO-d₆) δ 9.92 (br s, 1H), 9.09 (s, 1H), 9.02-8.89 (m, 1H), 8.21 (d, J=7.6 Hz, 1H), 7.66 (d, J=8.4 Hz, 1H), 7.51-7.25 (m, 6H), 7.11 (d, J=7.2 Hz, 1H), 6.97 (d, J=2.4 Hz, 1H), 6.15 (s, 1H), 5.14-4.82 (m, 2H), 4.55-4.38 (m, 5H), 4.27 (br s, 1H), 3.71-3.52 (m, 7H), 3.29-3.18 (m, 3H), 2.99-2.87 (m, 2H), 2.78-2.58 (m, 6H), 2.48-2.40 (m, 4H), 2.35-2.00 (m, 8H), 1.89-1.52 (m, 9H), 1.48-1.31 (m, 3H), 1.00-0.73 (m, 12H).

Exemplary Synthesis of (2S,4R)-1-[(2S)-2-{3-[(3S)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-3-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 196)

Step 1: Preparation of tert-butyl 4-fluoro-4-[[(2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]piperidine-1-carboxylate

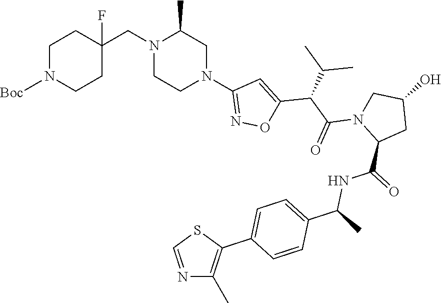 [SMILES: Cc1ncsc1-c1ccc([C@H](C)NC(=O)[C@@H]2C[C@@H](O)CN2C(=O)[C@H](c2cc(N3CCN(CC4(F)CCN(C(=O)OC(C)(C)C)CC4)[C@@H](C)C3)no2)C(C)C)cc1]

To a solution of tert-butyl 4-fluoro-4-formyl-piperidine-1-carboxylate (1.2 g, 5.2 mmol, 5.0 eq) and (2S,4R)-4-hydroxy-1-[(2S)-3-methyl-2-[3-[(3S)-3-methylpiperazin-1-yl]isoxazol-5-yl]butanoyl]-N-[(1S)-1-[4-(4-methylthiazol-5-yl)phenyl]ethyl]pyrrolidine-2-carboxamide (602 mg, 1 mmol, 1.0 eq) in CH₂Cl₂ (6 mL) and i-PrOH (6 mL) were added AcOH (0.24 mL, 4 mmol, 4.0 eq) and 2-methylpyridine borane (555 mg, 5 mmol, 5.0 eq). The mixture was stirred at 50° C. for 20 h, then concentrated in vacuum. The crude product was purified by column chomatography (0-7% methanol in dichloromethane) to afford tert-butyl 4-fluoro-4-[[(2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]piperidine-1-carboxylate (230 mg, 28%) as a yellow solid. MS (ESI) m/z: 796.3 [M+1]⁺.

Step 2: Preparation of (2S,4R)-1-[(2S)-2-[3-[(3S)-4-[(4-fluoro-4-piperidyl)methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

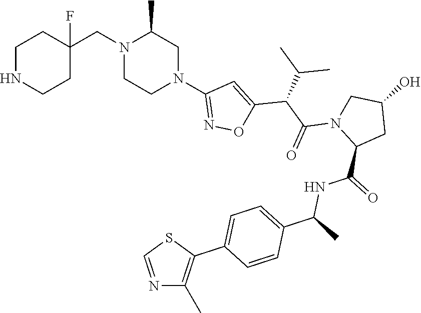 [SMILES: Cc1ncsc1-c1ccc([C@H](C)NC(=O)[C@@H]2C[C@@H](O)CN2C(=O)[C@H](c2cc(N3CCN(CC4(F)CCNCC4)[C@@H](C)C3)no2)C(C)C)cc1]

To a solution of tert-butyl 4-fluoro-4-[[(2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]piperidine-1-carboxylate (230 mg, 0.3 mmol, 1.0 eq) in CH₂Cl₂ (2 mL) was added 4 M HCl in dioxane (2.2 mL), and the reaction mixture was stirred at 25° C. for 0.5 h. The reaction mixture was suspended in petroleum ether (40 mL) and filtered, the filter cake was dissolved with tetrahydrofuran (20 mL) and then basified with triethylamine till pH 9. The mixture was filtered and the filtrate was concentrated under reduced pressure to give (2S,4R)-1-[(2S)-2-[3-[(3S)-4-[(4-fluoro-4-piperidyl)methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (198 mg, 98%) as a yellow solid. MS (ESI) m/z: 696.4 [M+1]⁺.

Step 3: Preparation of tert-butyl 3-[7-(8-ethyl-3-hydroxy-1-naphthyl)-8-fluoro-2-[2-[[4-fluoro-4-[[(2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]-1-piperidyl]ethoxy]pyrido[4,3-d]pyrimidin-4-yl]-3,8-diazabicyclo[3.2.1]octane-8-carboxylate

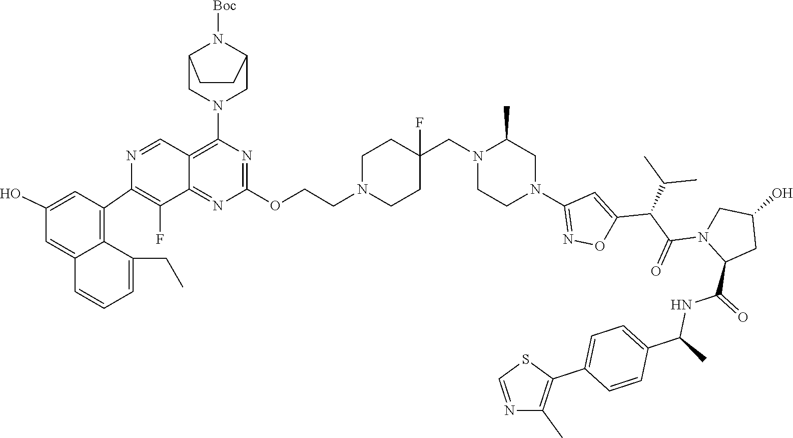 [SMILES: CCc1cccc2cc(O)cc(-c3ncc4c(N5CC6CCC(C5)N6C(=O)OC(C)(C)C)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)C[C@@H]6C)CC5)nc4c3F)c12]

To a solution of (2S,4R)-1-[(2S)-2-[3-[(3S)-4-[(4-fluoro-4-piperidyl)methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (198 mg, 0.28 mmol, 1.0 eq) and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (167 mg, 0.28 mmol, 1.0 eq) in CH₂Cl₂ (4 mL) and i-PrOH (4 mL) were added AcOH (650 uL, 1.14 mmol, 4.0 eq) and 2-methylpyridine borane (152 mg, 1.42 mmol, 5.0 eq). The mixture was stirred at 25° C. for 2 h, then concentrated in vacuum. The crude product was purified by column chromatography (0˜5% methanol in dichloromethane) to afford tert-butyl 3-[7-(8-ethyl-3-hydroxy-1-naphthyl)-8-fluoro-2-[2-[[4-fluoro-4-[(2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]-1-piperidyl]ethoxy]pyrido[4,3-d]pyrimidin-4-yl]-3,8-diazabicyclo[3.2.1]octane-8-carboxylate (270 mg, 75%) as a yellow solid. MS (ESI) m/z: 1267.4 [M+1]⁺.

Step 4: Preparation of (2S,4R)-1-[(2S)-2-[3-[(3S)-4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

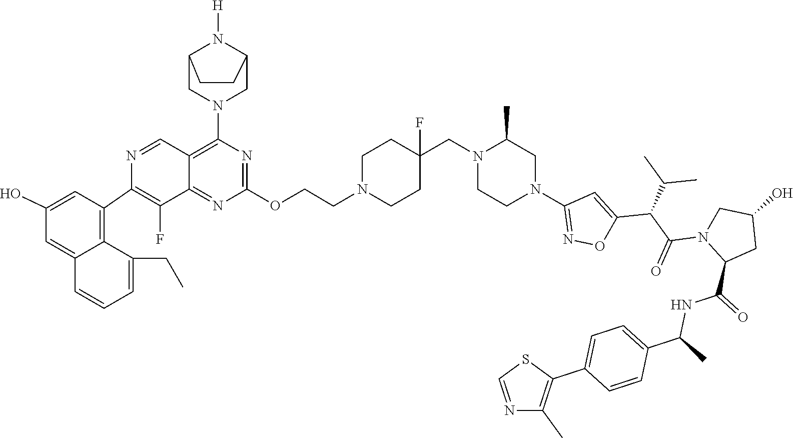 [SMILES: CCc1cccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)C[C@@H]6C)CC5)nc4c3F)c12]

To a reaction mixture of tert-butyl 3-[7-(8-ethyl-3-hydroxy-1-naphthyl)-8-fluoro-2-[2-[[4-fluoro-4-[[(2S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-2-methyl-piperazin-1-yl]methyl]-1-piperidyl]ethoxy]pyrido[4,3-d]pyrimidin-4-yl]-3,8-diazabicyclo[3.2.1]octane-8-carboxylate (270 mg, 0.21 mmol, 1.0 eq) in CH₂Cl₂ (2 mL) was added 4 M HCl/dioxane (1.6 mL, 30.0 eq). The resulting suspension was stirred at 25° C. for 0.5 h. The reaction mixture was diluted with petroleum ether (40 mL) and filtered. The filter cake was dissolved with THF (20 mL) and basified with triethylamine till pH 9. The mixture was filtered, and the filtrate was concentrated under reduced pressure. The crude product was purified by prep-HPLC (column: Xtimate C18 150*40 mm*10 um; mobile phase: [water (FA)-ACN]; B %: 0%-34%, 36 min) to give (2S,4R)-1-[(2S)-2-[3-[(3S)-4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (75.9 mg, formic aicd salt, 29%) as a white solid. MS (ESI) m/z: 1167.7 [M+1]⁺; ¹H NMR (400 MHz, CD₃OD) δ 9.08 (s, 1H), 8.90-8.81 (m, 1H), 8.46 (s, 1H), 7.62 (d, J=8.4 Hz, 1H), 7.41-7.26 (m, 6H), 7.16 (d, J=7.2 Hz, 1H), 7.00 (d, J=2.4 Hz, 1H), 6.17-6.03 (m, 1H), 4.98 (d, J=7.2 Hz, 1H), 4.82-4.68 (m, 5H), 4.57 (t, J=8.0 Hz, 1H), 4.43 (s, 1H), 4.07 (d, J=10.8 Hz, 2H), 3.9G4-3.80 (m, 2H), 3.76-3.57 (m, 3H), 3.39-3.32 (m, 1H), 3.26-3.17 (m, 4H), 3.09-2.93 (m, 2H), 2.90-2.69 (m, 4H), 2.57 (d, J=4.4 Hz, 1H), 2.50-2.39 (m, 5H), 2.38-2.18 (m, 4H), 2.14-1.72 (m, 9H), 1.60-1.44 (m, 3H), 1.08-0.94 (m, 6H), 0.93-0.82 (m, 6H).

Exemplary Synthesis of (2S,4R)-1-[(2R)-2-(3-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-4-fluoropiperidin-4-yl)methyl]piperazin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 198)

Step 1: Preparation of tert-butyl 3-[8-fluoro-2-[2-[[4-fluoro-4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate

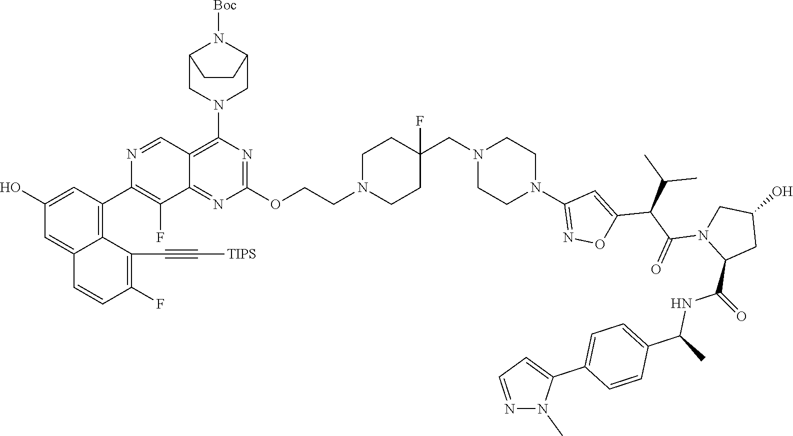

To a solution of tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (285 mg, 0.376 mmol, 1.0 eq) and (2S,4R)-1-[(2R)-2-[3-[4-[(4-fluoro-4-piperidyl)methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (250 mg, 0.376 mmol, 1.0 eq) in CH₂Cl₂ (20 mL) were added HOAc (113 mg, 1.88 mmol, 5.0 eq) and 2-picoline borane complex (201 mg, 1.88 mmol, 5.0 eq). The mixture was stirred at 25° C. for 1 h, then concentrated. The residue was purified by column chromatography (0-8% MeOH in CH₂Cl₂) to afford tert-butyl 3-[8-fluoro-2-[2-[[4-fluoro-4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (280 mg, 47%) as yellow solid. MS (ESI) m/z: 1406.5. [M+1]⁺.

Step 2: Preparation of tert-butyl 3-[7-(8-ethynyl-7-fluoro-3-hydroxy-1-naphthyl)-8-fluoro-2-[2-[[4-fluoro-4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate

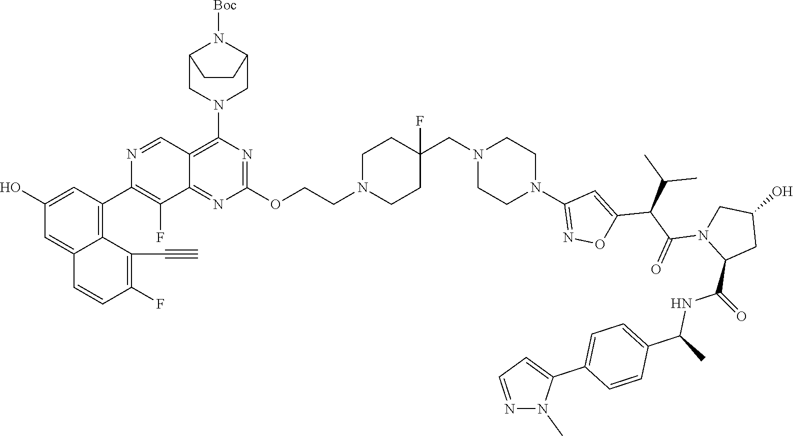 [SMILES: C#Cc1c(F)ccc2cc(O)cc(-c3ncc4c(N5CC6CCC(C5)N6C(=O)OC(C)(C)C)nc(OCCN5CCC(F)(CN6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12]

To a solution of tert-butyl 3-[8-fluoro-2-[2-[[4-fluoro-4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]-7-[7-fluoro-3-hydroxy-8-(2-triisopropylsilylethynyl)-1-naphthyl]pyrido[4,3-d]pyrimidin-4-yl]-3,8-diazabicyclo[3.2.1]octane-8-carboxylate (280 mg, 0.2 mmol, 1.0 eq) in THF (5 mL) was added TBAF (1 M, 400 μL, 2.0 eq). The mixture was stirred at 25° C. for 15 h, then diluted with ethyl acetate (50 mL) and washed with water (30 mL×2). The organic layer was washed with brine (30 mL), dried over Na₂SO₄, filtered, and concentrated under reduced pressure to afford tert-butyl 3-[7-(8-ethynyl-7-fluoro-3-hydroxy-1-naphthyl)-8-fluoro-2-[2-[[4-fluoro-4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (250 mg, crude) as yellow solid, which was used in the next step directly. MS (ESI) m/z: 1251.6. [M+1]⁺.

Step 3: Preparation of (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-fluoro-4-piperidyl]methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

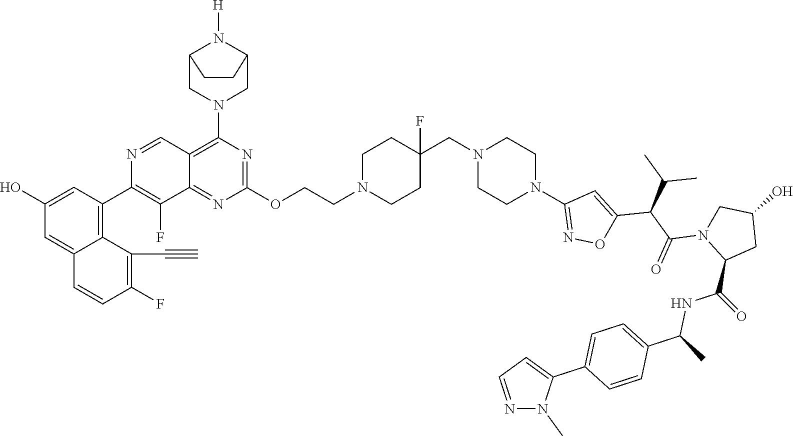 [SMILES: C#Cc1c(F)ccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12]

A solution of tert-butyl 3-[7-(8-ethynyl-7-fluoro-3-hydroxy-1-naphthyl)-8-fluoro-2-[2-[[4-fluoro-4-[[4-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (250 mg, 0.2 mmol, 1.0 eq) in HCOOH (3.0 mL) was stirred at 25° C. for 0.5 h. The mixture was suspended in petroleum ether (50 mL), filtered, and dissolved in MeOH (15 mL). Adjusted the pH with trimethylamine to 8, concentrated under reduced pressure to. The residue was purified by prep-HPLC (Eluent: 26˜66% acetonitrile in water; Gradient time: 36 min; Hold time: 3 min; Flow rate: 25 mL/min) to afford (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-fluoro-4-piperidyl]methyl]piperazin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (amount?). MS (ESI) m/z: 1150.7 [M+1]⁺; 1H NMR (400 MHz, CD₃OD) δ 9.01 (s, 1H), 7.88-7.85 (m, 1H), 7.48 (d, J=2.0 Hz, 1H), 7.45-7.39 (m, 4H), 7.36-7.30 (m, 2H), 7.20 (d, J=2.0 Hz, 1H), 6.34 (d, J=2.0 Hz, 1H), 6.08-6.03 (m, 1H), 5.04 (q, J=6.0 Hz, 1H), 4.71-4.62 (m, 5H), 4.51 (t, J=8.0 Hz, 1H), 4.44-4.43 (m, 1H), 3.85-3.82 (m, 4H), 3.75-3.72 (m, 4H), 3.64-3.59 (m, 2H), 3.37 (s, 1H), 3.21 (t, J=2.0 Hz, 4H), 2.96-2.89 (m, 4H), 2.63 (t, J=4.8 Hz, 4H), 2.56-2.42 (m, 4H), 2.41-2.30 (m, 1H), 2.20-2.15 (m, 1H), 1.99-1.81 (m, 8H), 1.59-1.52 (m, 3H), 1.05 (d, J=6.4 Hz, 3H), 0.92-0.88 (m, 3H).

Exemplary Synthesis of (2S,4R)-1-[(2R)-2-{3-[(3R)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-3-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 200)

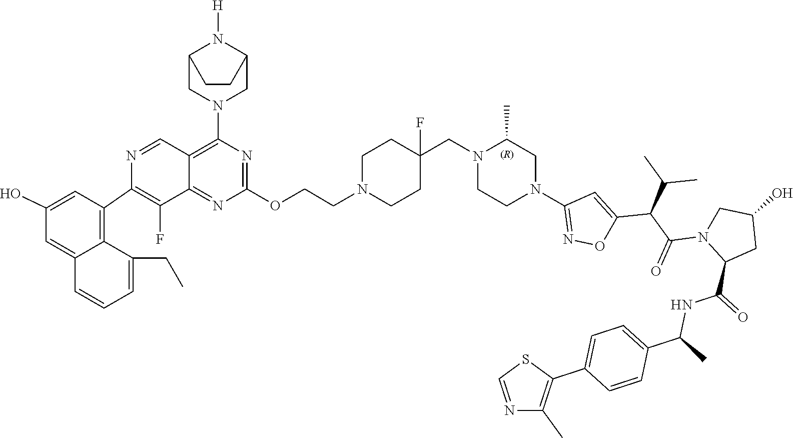

The title compound was prepared analogously to (2S,4R)-1-[(2S)-2-{3-[(3R)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-3-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-1-[(2R)-2-[3-[(3R)-4-[(4-fluoro-4-piperidyl)methyl]-3-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). MS (ESI) m/z: 1167.7 [M+H]⁺; ¹H NMR (400 MHz, DMSO-d₆) δ 9.10 (s, 1H), 8.98 (s, 1H), 8.41 (d, J=7.6 Hz, 1H), 7.66 (d, J=8.0 Hz, 1H), 7.47-7.41 (m, 2H), 7.39-7.33 (m, 3H), 7.28 (d, J=2.4 Hz, 1H), 7.12 (d, J=7.2 Hz, 1H), 6.96 (d, J=2.4 Hz, 1H), 6.15-6.00 (m, 1H), 4.91 (quin, J=7.2 Hz, 1H), 4.51-4.42 (m, 4H), 4.36 (t, J=8.0 Hz, 1H), 4.28 (br s, 1H), 3.73-3.64 (m, 6H), 3.47-3.25 (m, 6H), 2.99-2.90 (m, 2H), 2.78-2.63 (m, 6H), 2.45 (s, 3H), 2.42-2.12 (m, 8H), 2.06-1.97 (m, 1H), 1.91-1.58 (m, 9H), 1.47-1.33 (m, 3H), 0.96 (dd, J=6.4, 16.0 Hz, 6H), 0.85-0.76 (m, 6H).

Exemplary Synthesis of (2S,4R)-1-[(2S)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]piperazin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 201)

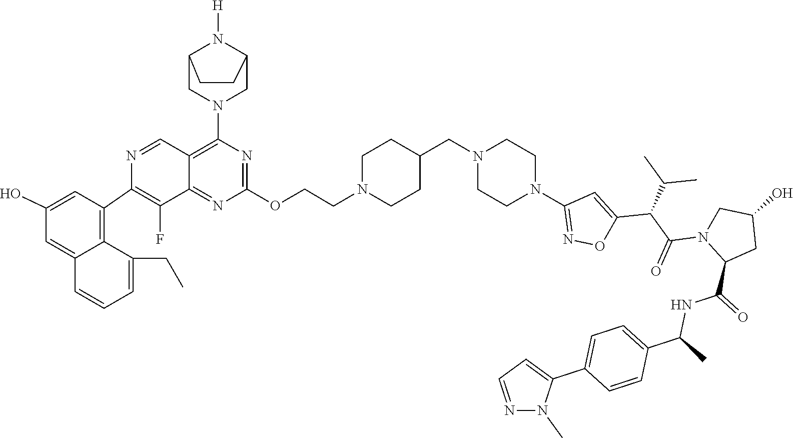 [SMILES: CCc1cccc2cc(O)cc(-c3ncc4c(N5CC6CCC(C5)N6)nc(OCCN5CCC(CN6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12]

The title compound was made in an analogous manner to (2S,4R)-1-[(2R)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]piperazin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide, starting from tert-butyl 4-[[5-[(1S)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]piperazine-1-carboxylate and (2S,4R)-4-hydroxy-1-[(2S)-3-methyl-2-[3-[4-(4-piperidylmethyl) piperazin-1-yl]isoxazol-5-yl]butanoyl]-N-[(1S)-1-[4-(2-methylpyrazol-3-yl)phenyl]ethyl]pyrrolidine-2-carboxamide. (white solid). MS (ESI) m/z: 1118.6 [M+H]⁺; ¹H NMR (400 MHz, CD₃OD) δ 9.04 (s, 1H), 7.62 (d, J=8.0 Hz, 1H), 7.50-7.32 (m, 6H), 7.28 (d, J=2.4 Hz, 1H), 7.16 (d, J=7.2 Hz, 1H), 7.01 (d, J=2.4 Hz, 1H), 6.38-6.28 (m, 1H), 6.16-6.02 (m, 1H), 5.02-4.97 (m, 1H), 4.67-4.55 (m, 7H), 4.42 (s, 1H), 3.89-3.81 (m, 3H), 3.77-3.60 (m, 7H), 3.23-3.19 (m, 3H), 3.17-3.10 (m, 2H), 2.93 (t, J=5.2 Hz, 2H), 2.53-2.43 (m, 4H), 2.40-2.18 (m, 8H), 1.98-1.95 (m, 1H), 1.90-1.75 (m, 6H), 1.49 (d, J=7.2 Hz, 3H), 1.33-1.23 (m, 2H), 1.05 (d, J=6.4 Hz, 3H), 0.93-0.83 (m, 6H).

Exemplary Synthesis of (2S,4R)-1-[(2R)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]piperidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 202)

Step 1: Preparation of tert-butyl 3-[7-(8-ethyl-3-hydroxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate

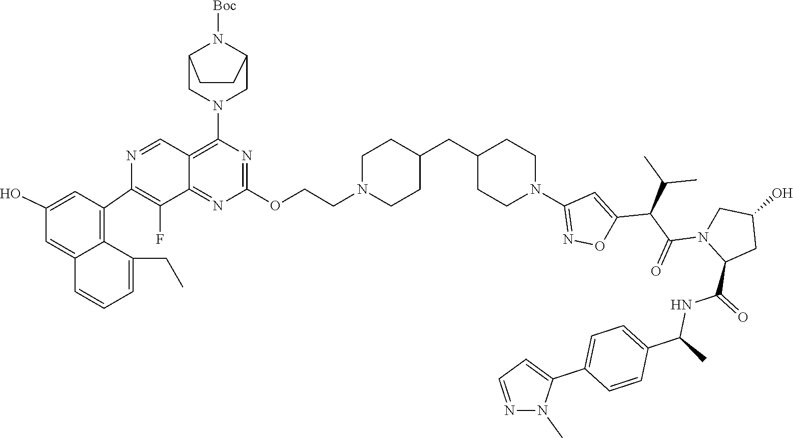 [SMILES: CCc1cccc2cc(O)cc(-c3ncc4c(N5CC6CCC(C5)N6C(=O)OC(C)(C)C)nc(OCCN5CCC(CC6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (325 mg, 387 umol) and (2S,4R)-4-hydroxy-1-[(2R)-3-methyl-2-[3-[4-(4-piperidylmethyl)-1-piperidyl]isoxazol-5-yl]butanoyl]-N-[(1S)-1-[4-(2-methylpyrazol-3-yl)phenyl]ethyl]pyrrolidine-2-carboxamide (250 mg, 387 umol) in CH₂Cl₂ (4 mL) and i-PrOH (4 mL) was added 2-methylpyridine borane (207.02 mg, 1.9 mmol) and AcOH (116 mg, 1.9 mmol, 110.7 uL). The mixture was stirred at 25° C. for 1 h. Triethylamine was added until pH 8 and then concentrated under reduced pressure. The residue was purified by flash column chromatography (0-10% methanol in dichloromethane) to afford tert-butyl 3-[7-(8-ethyl-3-hydroxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (380 mg, 75%) as a yellow solid. MS (ESI) m/z: 1217.4 [M+1]⁺.

Step 2: Preparation of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

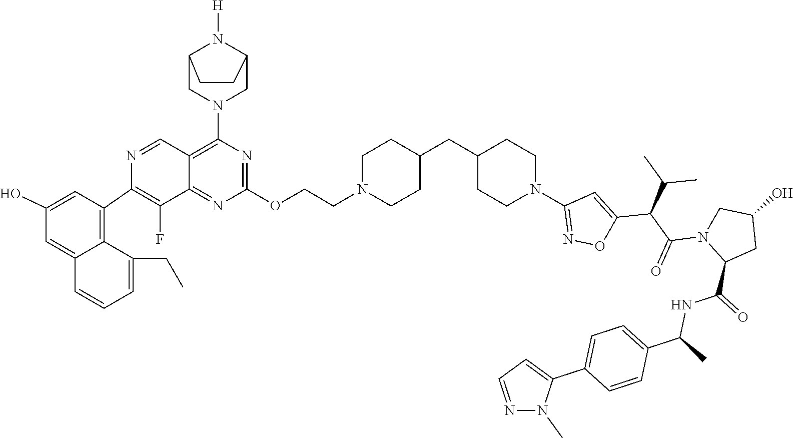 [SMILES: CCc1cccc2cc(O)cc(-c3ncc4c(N5CC6CCC(C5)N6)nc(OCCN5CCC(CC6CCN(c7cc([C@H](C(=O)N8C[C@H](O)C[C@H]8C(=O)N[C@@H](C)c8ccc(-c9ccnn9C)cc8)C(C)C)on7)CC6)CC5)nc4c3F)c12]

To a solution of tert-butyl 3-[7-(8-ethyl-3-hydroxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (380 mg, 312 umol) in CH₂Cl₂ (5 mL) was added HCl in dioxane (4 M, 4 mL). The mixture was stirred at 0° C. for 30 min, then suspended in petroleum ether (20 mL). The solid material was filtered and dissolved with THE, adjusted the pH with triethylamine until 8. The mixture was filtered, and the filtrate was concentrated in vacuum. The residue was purified by prep-HPLC (column: Xtimate C18 150*40 mm*10 um; mobile phase: [water (FA)-ACN]; B %: 0%-38%, 36 min) to afford (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (130.0 mg, 36%, di-formic acid salt) as a white solid. MS (ESI) m/z: 1118.0 [M+1]⁺; ¹H NMR (400 MHz, CD₃OD) δ 9.10 (s, 1H), 8.60-8.49 (m, 1H), 8.56-8.46 (m, 1H), 7.65 (d, J=8.0 Hz, 1H), 7.50-7.48 (m, 1H), 7.47-7.35 (m, 5H), 7.31 (d, J=3.6 Hz, 1H), 7.18 (d, J=7.6 Hz, 1H), 7.01 (d, J=4.0 Hz, 1H), 6.36-6.34 (m, 1H), 6.10-6.03 (m, 1H), 5.09-5.01 (m, 1H), 4.80-4.60 (m, 6H), 4.55-4.42 (m, 2H), 3.92-3.72 (m, 8H), 3.70-3.57 (m, 4H), 3.55-3.45 (m, 2H), 2.88-2.70 (m, 4H), 2.42-2.14 (m, 4H), 2.02-1.87 (m, 6H), 1.77-1.64 (m, 3H), 1.62-1.51 (m, 4H), 1.47-1.31 (m, 2H), 1.29-1.16 (m, 4H), 1.06 (d, J=8.0 Hz, 3H), 0.95-0.85 (m, 6H).

Exemplary Synthesis of (2S,4R)-1-[(2S)-2-{3-[(2S)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-2-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 203)

Step 1: Preparation of tert-butyl 4-fluoro-4-[[(3S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]piperidine-1-carboxylate

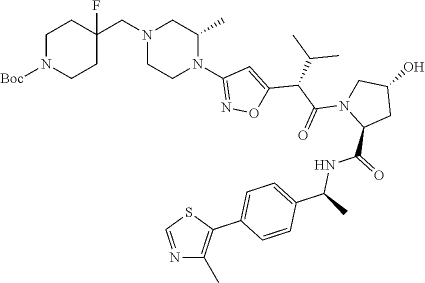 [SMILES: Cc1ncsc1-c1ccc([C@H](C)NC(=O)[C@@H]2C[C@@H](O)CN2C(=O)[C@H](c2cc(N3CCN(CC4(F)CCN(C(=O)OC(C)(C)C)CC4)C[C@@H]3C)no2)C(C)C)cc1]

To a reaction mixture of tert-butyl 4-fluoro-4-formyl-piperidine-1-carboxylate (424 mg, 1.8 mmol) and (2S,4R)-4-hydroxy-1-[(2S)-3-methyl-2-[3-[(2S)-2-methylpiperazin-1-yl]isoxazol-5-yl]butanoyl]-N-[(1S)-1-[4-(4-methylthiazol-5-yl)phenyl]ethyl]pyrrolidine-2-carboxamide (213 mg, 367 umol) in CH₂Cl₂ (10 mL) and i-PrOH (3 mL) was added HOAc (84 uL, 1.5 mmol) and 2-methylpyridine borane (196 mg, 1.8 mmol), the reaction was stirred at 25° C. for 15 h, then concentrated under reduced pressure. The residue was purified by flash column chromatography (0-4% then 6% MeOH in CH₂Cl₂) to afford tert-butyl 4-fluoro-4-[[(3S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]piperidine-1-carboxylate (232 mg, 51%) as a colorless oil. MS (ESI) m/z: 796.1 [M+H]⁺.

Step 2: Preparation of (2S,4R)-1-[(2S)-2-[3-[(2S)-4-[(4-fluoro-4-piperidyl)methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

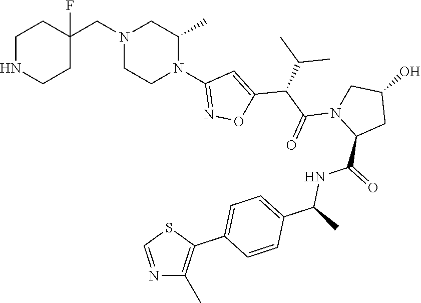 [SMILES: Cc1ncsc1-c1ccc([C@H](C)NC(=O)[C@@H]2C[C@@H](O)CN2C(=O)[C@H](c2cc(N3CCN(CC4(F)CCNCC4)C[C@@H]3C)no2)C(C)C)cc1]

To a reaction mixture of tert-butyl 4-fluoro-4-[[(3S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]piperidine-1-carboxylate (232 mg, 186 umol) in CH₂Cl₂ (2 mL) was added HCl in dioxane (4 M, 2 mL), the resulting suspension was stirred at 25° C. for 15 min. The reaction was diluted with petroleum ether (30 mL), the yellow precipitate was collected, dissolved with THF (30 mL), adjusted the pH with triethylamine to 8. The mixture was filtered and the solvent was concentrated under reduced pressure to afford (2S,4R)-1-[(2S)-2-[3-[(2S)-4-[(4-fluoro-4-piperidyl)methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (129.8 mg, crude) as a white solid, which was used in the next step directly. MS (ESI) m/z: 696.3 [M+H]⁺.

Step 3: Preparation of tert-butyl 3-[7-(8-ethyl-3-hydroxy-1-naphthyl)-8-fluoro-2-[2-[[4-fluoro-4-[[(3S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]-1-piperidyl]ethoxy]pyrido[4,3-d]pyrimidin-4-yl]-3,8-diazabicyclo[3.2.1]octane-8-carboxylate

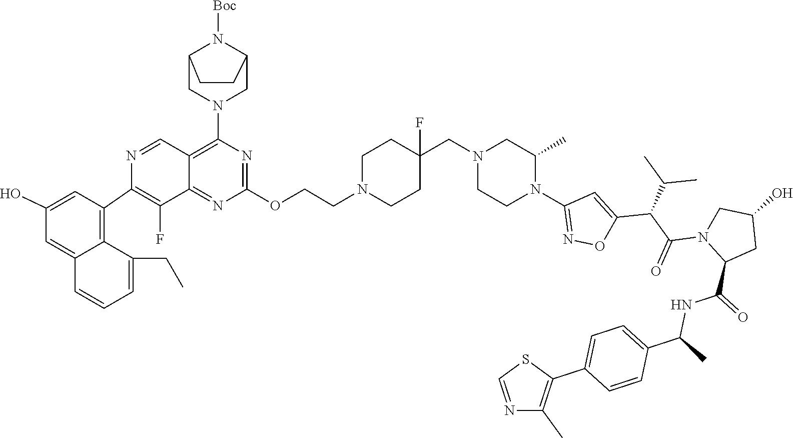 [SMILES: CCc1cccc2cc(O)cc(-c3ncc4c(N5CC6CCC(C5)N6C(=O)OC(C)(C)C)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)[C@@H](C)C6)CC5)nc4c3F)c12]

To a reaction mixture of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (235 mg, 280 umol) and (2S,4R)-1-[(2S)-2-[3-[(2S)-4-[(4-fluoro-4-piperidyl)methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (129.8 mg, 186 umol) in CH₂Cl₂ (6 mL) and i-PrOH (1 mL) was added HOAc (43.0 uL, 746 umol) and 2-methylpyridine borane (100 mg, 933 umol), the resulting suspension was stirred at 25° C. for 15 h, then concentrated under reduced pressure. The residue was purified by flash column chromatography (0-6% then up to 10% MeOH in CH₂Cl₂) to afford tert-butyl 3-[7-(8-ethyl-3-hydroxy-1-naphthyl)-8-fluoro-2-[2-[[4-fluoro-4-[[(3S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]-1-piperidyl]ethoxy]pyrido[4,3-d]pyrimidin-4-yl]-3,8-diazabicyclo[3.2.1]octane-8-carboxylate (319 mg, 94%) as a white solid. MS (ESI) m/z: 1267.6 [M+H]⁺.

Step 4: Preparation of (2S,4R)-1-[(2S)-2-[3-[(2S)-4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

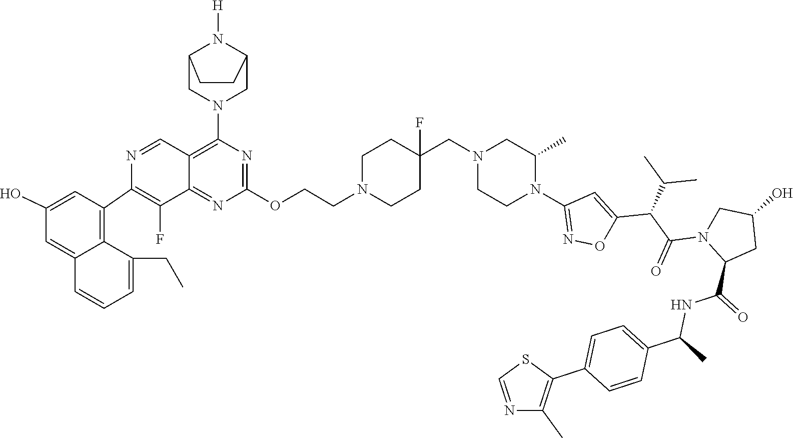 [SMILES: CCc1cccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)[C@@H](C)C6)CC5)nc4c3F)c12]

To a reaction mixture of tert-butyl 3-[7-(8-ethyl-3-hydroxy-1-naphthyl)-8-fluoro-2-[2-[[4-fluoro-4-[[(3S)-4-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazin-1-yl]methyl]-1-piperidyl]ethoxy]pyrido[4,3-d]pyrimidin-4-yl]-3,8-diazabicyclo[3.2.1]octane-8-carboxylate (319 mg, 176 umol) in CH₂Cl₂ (3 mL) was added HCl in dioxane (4 M, 3 mL), the resulting suspension was stirred at 25° C. for 0.25 h. The reaction mixture was diluted with petroleum ether (30 mL), the yellow precipitate was separated, dissolved in THF (30 mL), adjusted the pH with triethylamine to 8. The mixture was filtered, and the solvent was concentrated under reduced pressure. The crude product was purified by prep-HPLC (column: Xtimate C18 150*40 mm*10 um; mobile phase: [water (formic acid)-acetonitrile]; Begin B: 0; End B: 38; Gradient Time (min): 36; 100% B Hold Time (min): 3; Flow Rate (ml/min): 60), the crude product was further purified by SFC (column: DAICEL CHIRALPAK IE (250 mm*30 mm, 10 um); mobile phase: [acetonitrile/ethanol (0.1% NH₃H₂O)]; Begin B: 55 End B: 55; Gradient Time (min): 100% B Hold Time (min): Flow Rate (ml/min): 80) to afford (2S,4R)-1-[(2S)-2-[3-[(2S)-4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methyl]-2-methyl-piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (69.0 mg, 32%, formic acid salt) as a white solid. MS (ESI) m/z: 1167.7 [M+H]⁺; ¹H NMR (400 MHz, DMSO-d₆) δ 9.08 (s, 1H), 9.00-8.89 (m, 1H), 8.22 (d, J=7.6 Hz, 1H), 7.66 (d, J=8.4 Hz, 1H), 7.48-7.27 (m, 6H), 7.11 (d, J=7.2 Hz, 1H), 6.96 (d, J=2.4 Hz, 1H), 6.10-6.07 (m, 1H), 4.91-4.81 (m, 1H), 4.50-4.38 (m, 5H), 4.30-4.23 (m, 1H), 3.70-3.52 (m, 10H), 2.74-2.67 (m, 5H), 2.47-2.43 (m, 4H), 2.41-2.11 (m, 10H), 2.10-1.85 (m, 2H), 1.79 (ddd, J=4.8, 7.2, 12.4 Hz, 3H), 1.75-1.55 (m, 7H), 1.46-1.33 (m, 3H), 1.14-1.06 (m, 3H), 0.95 (d, J=6.4 Hz, 2H), 0.83-0.73 (m, 7H).

Exemplary Synthesis of (2S,4R)-1-[(2R)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-hydroxypiperidin-4-yl)methyl]piperidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 204)

Step 1: Preparation of tert-butyl 4-hydroxy-4-(4-piperidylmethyl)piperidine-1-carboxylate

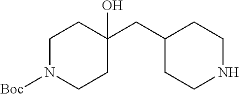 [SMILES: CC(C)(C)OC(=O)N1CCC(O)(CC2CCNCC2)CC1]

To a solution of tert-butyl 4-hydroxy-4-(4-pyridylmethyl)piperidine-1-carboxylate (3.0 g, 10 mmol) in ethyl alcohol (60 mL) was added acetic acid (1.76 mL, 31 mmol) and PtO₂ (0.70 g, 3 mmol). The mixture was degassed under vacuum and purged with H₂ several times and stirred at 70° C. under H₂ (50 psi) for 16 h. The reaction mixture was filtered with Celite, and the filtrate was concentrated in vacuum. The residue was dissolved with water (30 mL) and adjusted the pH to 10 by saturated aqueous sodium carbonate solution. The mixture was lyophilized to afford crude product, which was dissolved with dichloromethane (30 mL). The mixture was filtered and the filter cake was washed with dichloromethane (10 mL×3), the filtrate was concentrated in vacuum to give tert-butyl 4-hydroxy-4-(4-piperidylmethyl) piperidine-1-carboxylate (3.04 g, 99%) as a black brown oil, which was used directly in the next step. MS (ESI) m/z: 299.2 [M+1]⁺.

Step 2: Preparation of tert-butyl 4-hydroxy-4-[[1-[5-(1-methoxycarbonyl-2-methyl-propyl)isoxazol-3-yl]-4-piperidyl]methyl]piperidine-1-carboxylate

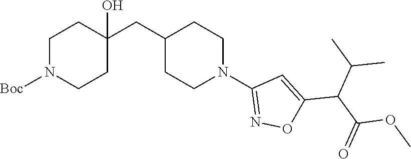 [SMILES: COC(=O)C(c1cc(N2CCC(CC3(O)CCN(C(=O)OC(C)(C)C)CC3)CC2)no1)C(C)C]

To a solution of tert-butyl 4-hydroxy-4-(4-piperidylmethyl)piperidine-1-carboxylate (1.5 g, 5 mmol) and 4 A MS (6.0 g) in DMA (20 mL) was added triethylamine (2.10 mL, 15 mmol) and methyl 3-methyl-2-[3-(1,1,2,2,3,3,4,4,4-nonafluorobutylsulfonyloxy)isoxazol-5-yl]butanoate (2.4 g, 5 mmol). The mixture was stirred at 130° C. for 3 h. The reaction mixture was diluted with ethyl acetate (40 mL) and filtered through Celite pad, the filter cake was rinsed with ethyl acetate (20 mL). The mixture was diluted with water (40 mL), the organic layer was separated, and the aqueous layer was extracted with ethyl acetate (20 mL×3). The combined organic layers were washed with water (20 mL×5), brine (40 mL×3), dried over Na₂SO₄, filtered, and concentrated under reduced pressure. The crude product was purified by flash column chromatography (0-30% ethyl acetate in petroleum ether) to afford tert-butyl 4-hydroxy-4-[[1-[5-(1-methoxycarbonyl-2-methyl-propyl)isoxazol-3-yl]-4-piperidyl]methyl]piperidine-1-carboxylate (1.3 g, 53%) as a yellow oil. MS (ESI) m/z: 480.2 [M+1]⁺.

Step 3: Preparation of 2-[3-[4-[(1-tert-butoxycarbonyl-4-hydroxy-4-piperidyl)methyl]-1-piperidyl]isoxazol-5-yl]-3-methyl-butanoic acid

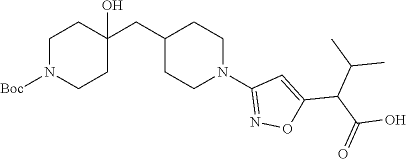 [SMILES: CC(C)C(C(=O)O)c1cc(N2CCC(CC3(O)CCN(C(=O)OC(C)(C)C)CC3)CC2)no1]

To a solution of tert-butyl 4-hydroxy-4-[[1-[5-(1-methoxycarbonyl-2-methyl-propyl)isoxazol-3-yl]-4-piperidyl]methyl]piperidine-1-carboxylate (1.3 g, 2.7 mmol) in tetrahydrofuran (80 mL) and water (20 mL) was added LiOH·H₂O (559 mg, 13 mmol). The reaction mixture was stirred at 25° C. for 16 h, then concentrated in vacuum. The residue was treated with 2 M hydrochloric acid solution till pH around 3-4. The mixture was extracted with dichloromethane (20 mL×3). The combined organic layers were washed with brine (15 mL), dried over anhydrous Na₂SO₄, filtered and concentrated under reduced pressure to give 2-[3-[4-[(1-tert-butoxycarbonyl-4-hydroxy-4-piperidyl)methyl]-1-piperidyl]isoxazol-5-yl]-3-methyl-butanoic acid (1.2 g, 96%) as a yellow solid, which was used directly in the next step. MS (ESI) m/z: 466.0 [M+1]⁺.

Step 4: Preparation of tert-butyl 4-hydroxy-4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]piperidine-1-carboxylate

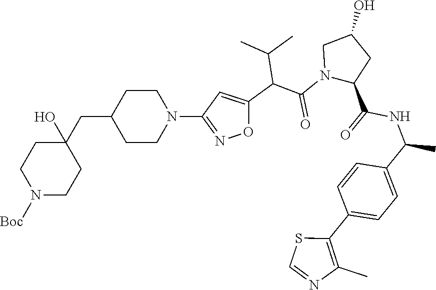 [SMILES: Cc1ncsc1-c1ccc([C@H](C)NC(=O)[C@@H]2C[C@@H](O)CN2C(=O)C(c2cc(N3CCC(CC4(O)CCN(C(=O)OC(C)(C)C)CC4)CC3)no2)C(C)C)cc1]

To a solution 2-[3-[4-[(1-tert-butoxycarbonyl-4-hydroxy-4-piperidyl)methyl]-1-piperidyl]isoxazol-5-yl]-3-methyl-butanoic acid (1.2 g, 2.6 mmol), (2S,4R)-4-hydroxy-N-[(1S)-1-[4-(4-methylthiazol-5-yl)phenyl]ethyl]pyrrolidine-2-carboxamide (948 mg, 2.9 mmol) and DIEA (4.49 mL, 26 mmol) in DMF (10 mL) was added HATU (1.1 g, 2.8 mmol). The mixture was stirred at 25° C. for 2 h, diluted with water (40 mL) and extracted with dichloromethane (30 mL×3). The combined organic phase was washed with brine (20 mL), dried over anhydrous sodium sulfate, filtered, and concentrated. The residue was purified by column chromatography (0-5% methanol in dichloromethane) to afford tert-butyl 4-hydroxy-4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]piperidine-1-carboxylate (1.3 g, 65%) as a light yellow solid. MS (ESI) m/z: 779.2 [M+1]⁺.

Step 5: Preparation of tert-butyl 4-hydroxy-4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]piperidine-1-carboxylate and tert-butyl 4-hydroxy-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]piperidine-1-carboxylate

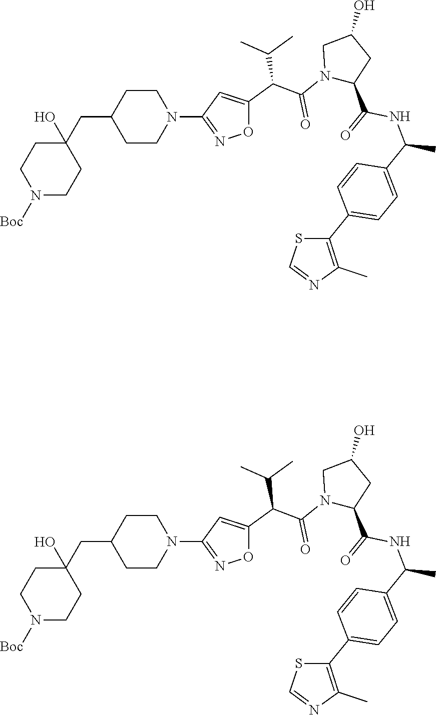 [SMILES: Cc1ncsc1-c1ccc([C@H](C)NC(=O)[C@@H]2C[C@@H](O)CN2C(=O)[C@H](c2cc(N3CCC(CC4(O)CCN(C(=O)OC(C)(C)C)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CC4(O)CCN(C(=O)OC(C)(C)C)CC4)CC3)no2)C(C)C)cc1]

Racemic tert-butyl 4-hydroxy-4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]piperidine-1-carboxylate (1.3 g) was separated by SFC (column: Daicel Chiral Pak IBN 250 mm*30 mm*10 um; mobile phase: [0.1% NH₃H₂O/ethanol]; B %: 45%-45%). Fraction 1 was obtained as tert-butyl 4-hydroxy-4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]piperidine-1-carboxylate (610 mg, 47%) as a light yellow solid. MS (ESI) m/z: 779.4 [M+1]⁺. Fraction 2 was obtained as tert-butyl 4-hydroxy-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]piperidine-1-carboxylate (510 mg, 39%) as a yellow solid. MS (ESI) m/z: 801.4 [M+Na]⁺.

Step 6: Preparation of (2S,4R)-4-hydroxy-1-[(2R)-2-[3-[4-[(4-hydroxy-4-piperidyl)methyl]-1-piperidyl]isoxazol-5-yl]-3-methyl-butanoyl]-N-[(1S)-1-[4-(4-methylthiazol-5-yl)phenyl]ethyl]pyrrolidine-2-carboxamide

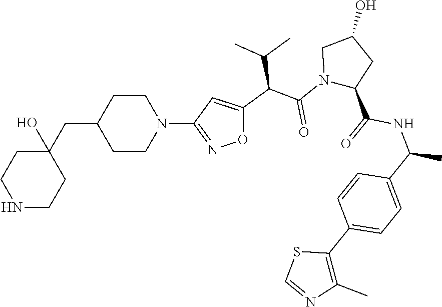 [SMILES: Cc1ncsc1-c1ccc([C@H](C)NC(=O)[C@@H]2C[C@@H](O)CN2C(=O)[C@@H](c2cc(N3CCC(CC4(O)CCNCC4)CC3)no2)C(C)C)cc1]

To a stirred solution of tert-butyl 4-hydroxy-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]piperidine-1-carboxylate (400 mg, 0.5 mmol) in dichloromethane (4 mL) was added 4 M HCl in dioxane (3.9 mL), and the reaction mixture was stirred at 25° C. for 0.5 h. The reaction mixture was diluted with petroleum ether (40 mL) and filtered. The filter cake was dissolved with tetrahydrofuran (20 ml) and then basified with triethylamine till pH 9. The mixture was filtered and the filtrate was concentrated under reduced pressure to give (2S,4R)-4-hydroxy-1-[(2R)-2-[3-[4-[(4-hydroxy-4-piperidyl)methyl]-1-piperidyl]isoxazol-5-yl]-3-methyl-butanoyl]-N-[(1S)-1-[4-(4-methylthiazol-5-yl)phenyl]ethyl]pyrrolidine-2-carboxamide (210 mg, 60%) as a yellow solid, which was used directly in the next step. MS (ESI) m/z: 679.1 [M+1]⁺.

Step 7: Preparation of tert-butyl 3-[7-(8-ethyl-3-hydroxy-1-naphthyl)-8-fluoro-2-[2-[[4-hydroxy-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate

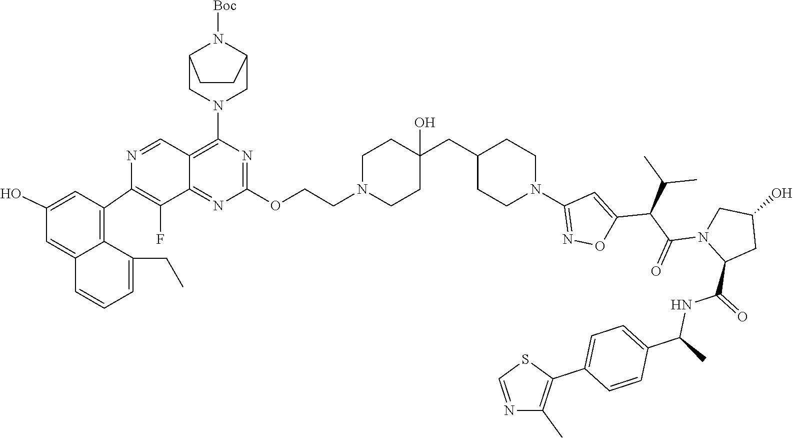 [SMILES: CCc1cccc2cc(O)cc(-c3ncc4c(N5CC6CCC(C5)N6C(=O)OC(C)(C)C)nc(OCCN5CCC(O)(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (180 mg, 0.3 mmol) and (2S,4R)-4-hydroxy-1-[(2R)-2-[3-[4-[(4-hydroxy-4-piperidyl)methyl]-1-piperidyl]isoxazol-5-yl]-3-methyl-butanoyl]-N-[(1S)-1-[4-(4-methylthiazol-5-yl)phenyl]ethyl]pyrrolidine-2-carboxamide (208 mg, 0.3 mmol) in dichloromethane (4 mL) and isopropyl alcohol (4 mL) were added acetic acid (0.07 mL, 1.2 mmol) and 2-methylpyridine borane (164 mg, 1.5 mmol). The mixture was stirred at 25° C. for 2 h, then concentrated in vacuum. The residue was purified by column chromatography (0-8% methanol in dichloromethane) to give tert-butyl 3-[7-(8-ethyl-3-hydroxy-1-naphthyl)-8-fluoro-2-[2-[[4-hydroxy-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (230 mg, 60%) as a yellow solid. MS (ESI) m/z: 1250.5 [M+1]⁺.

Step 8: Preparation of (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-hydroxy-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide

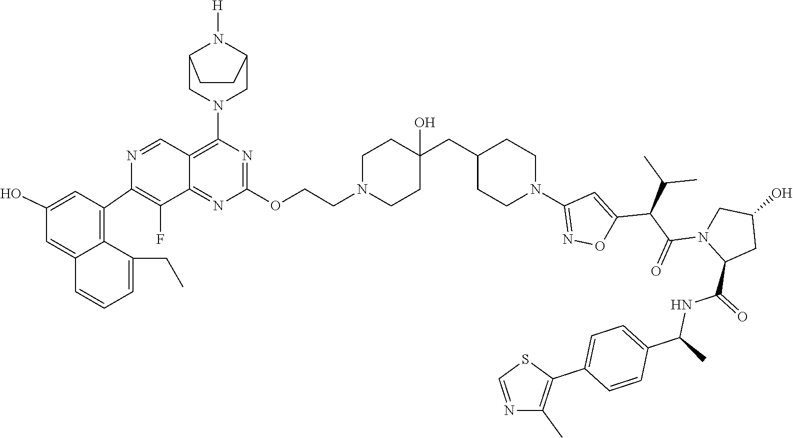 [SMILES: CCc1cccc2cc(O)cc(-c3ncc4c(N5CC6CCC(C5)N6)nc(OCCN5CCC(O)(CC6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a stirred solution of tert-butyl 3-[7-(8-ethyl-3-hydroxy-1-naphthyl)-8-fluoro-2-[2-[[4-hydroxy-4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-4-piperidyl]methyl]-1-piperidyl]ethoxy]pyrido[4,3-d]pyrimidin-4-yl]-3,8-diazabicyclo[3.2.1]octane-8-carboxylate (230 mg, 0.2 mmol) in dichloromethane (2 mL) was added 4 M HC in dioxane (1.4 mL), the reaction mixture was stirred at 25° C. for 0.5 h. The reaction was diluted with petroleum ether (40 mL) and filtered. The filter cake was dissolved in tetrahydrofuran (20 ml) and then basified with triethylamine till pH 9. The mixture was filtered, the filtrate was concentrated under reduced pressure. The crude product was purified by prep-HPLC (column: Xtimate C18 150*40 mm*10 um; mobile phase: [water (formic acid)-acetonitrle]; B %: 0%-36%, 36 min) to afford (2S,4R)-1-[(2R)-2-[3-[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-hydroxy-4-piperidyl]methyl]-1-piperidyl]isoxazol-5-yl]-3-methyl-butanoyl]-4-hydroxy-N-[(1S)-1-[4-(4-methylthiazol-5-yl)phenyl]ethyl]pyrrolidine-2-carboxamide (108.0 mg, formic acid salt, 49%) as a white solid. MS (ESI) m/z: 1151.0 [M+1]⁺; ¹H NMR (400 MHz, CD₃OD) δ 9.09 (s, 1H), 8.89-8.85 (m, 1H), 8.51 (s, 1H), 7.63 (d, J=8.4 Hz, 1H), 7.30 (d, J=2.4 Hz, 1H), 7.47-7.26 (m, 5H), 7.17 (d, J=7.2 Hz, 1H), 7.01 (d, J=2.8 Hz, 1H), 6.10-6.01 (m, 1H), 5.08-4.99 (m, 1H), 4.79-4.74 (m, 2H), 4.74-4.66 (m, 3H), 4.51 (t, J=8.4 Hz, 1H), 4.44 (d, J=1.6 Hz, 1H), 3.89-3.74 (m, 5H), 3.66-3.53 (m, 4H), 3.33 (s, 1H), 3.25 (d, J=10.4 Hz, 2H), 3.12-3.00 (m, 2H), 2.82 (t, J=12.4 Hz, 2H), 2.50-2.45 (m, 3H), 2.40-2.15 (m, 4H), 2.02-1.86 (m, 5H), 1.86-1.73 (m, 7H), 1.61-1.49 (m, 3H), 1.44 (d, J=5.2 Hz, 2H), 1.31-1.31 (m, 1H), 1.3-1.26 (m, 1H), 1.10-1.02 (m, 3H), 0.94-0.86 (m, 6H).

Exemplary Synthesis of (2S,4R)-1-[(2S)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-hydroxypiperidin-4-yl)methyl]piperidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 205)

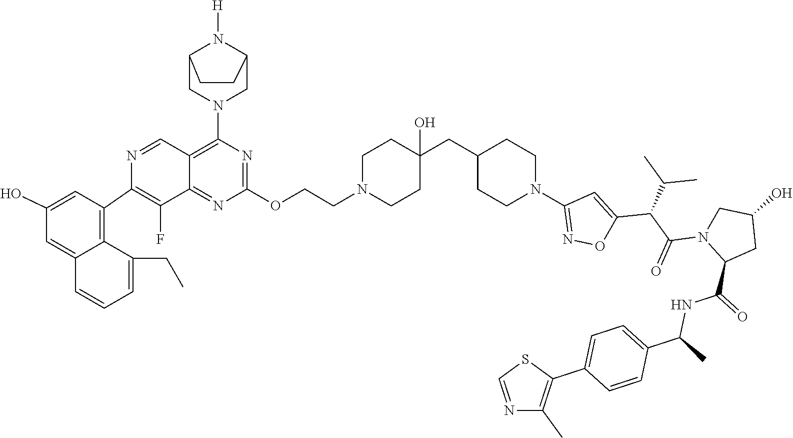 [SMILES: CCc1cccc2cc(O)cc(-c3ncc4c(N5CC6CCC(C5)N6)nc(OCCN5CCC(O)(CC6CCN(c7cc([C@@H](C(=O)N8C[C@H](O)C[C@H]8C(=O)N[C@@H](C)c8ccc(-c9scnc9C)cc8)C(C)C)on7)CC6)CC5)nc4c3F)c12]

The title compound was prepared analogously to (2S,4R)-1-[(2R)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-hydroxypiperidin-4-yl)methyl]piperidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide, starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2S)-2-[3-[4-[(4-hydroxy-4-piperidyl)methyl]-1-piperidyl]isoxazol-5-yl]-3-methyl-butanoyl]-N-[(1S)-1-[4-(4-methylthiazol-5-yl)phenyl]ethyl]pyrrolidine-2-carboxamide. (formic acid salt, white solid). MS (ESI) m/z: 1151.0 [M+1]⁺; 1H NMR (400 MHz, CD₃OD) δ 9.09 (s, 1H), 8.91-8.83 (m, 1H), 8.52 (s, 1H), 7.63 (d, J=8.2 Hz, 1H), 7.51-7.27 (m, 6H), 7.16 (d, J=6.8 Hz, 1H), 7.00 (d, J=2.4 Hz, 1H), 6.15-6.00 (m, 1H), 5.01-4.95 (m, 1H), 4.82-4.62 (m, 5H), 4.58 (t, J=8.0 Hz, 1H), 4.43 (s, 1H), 3.94-3.67 (m, 7H), 3.62-3.53 (m, 2H), 3.35 (s, 2H), 3.27 (s, 1H), 3.09 (d, J=7.2 Hz, 2H), 2.77 (t, J=12.4 Hz, 2H), 2.51-2.43 (m, 3H), 2.41-2.20 (m, 4H), 2.02-1.86 (m, 5H), 1.78-1.77 (m, 7H), 1.58-1.48 (d, J=7.2 Hz, 3H), 1.44-1.38 (m, 2H), 1.35-1.19 (m, 2H), 1.05 (d, J=6.8 Hz, 3H), 0.99-0.86 (m, 6H).

Exemplary Synthesis of (2S,4R)-1-[(2R)-2-(3-{4-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]piperazin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 206)

Step 1: Preparation of tert-butyl 4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate

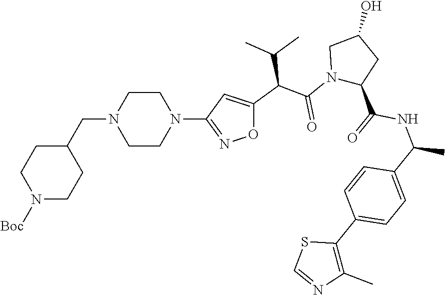 [SMILES: Cc1ncsc1-c1ccc([C@H](C)NC(=O)[C@@H]2C[C@@H](O)CN2C(=O)[C@@H](c2cc(N3CCN(CC4CCN(C(=O)OC(C)(C)C)CC4)CC3)no2)C(C)C)cc1]

To a mixture of (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide (350 mg, 0.6 mmol) and tert-butyl 4-formylpiperidine-1-carboxylate (263 mg, 1.2 mmol) in CH₂Cl₂ (4 mL) and i-PrOH (4 mL) were added AcOH (141 uL, 2.5 mmol) and 2-methylpyridine borane (330 mg, 3.1 mmol) at 25° C. The mixture was stirred at 25° C. for 1 h, then concentrated in vacuum. The pH of the residue was adjusted to 10 by trimethylamine and purified by column chromatography (0-3% then up to 5% methanol (1N NH₃ as additive) in dichloromethane) to afford tert-butyl 4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (536 mg, crude) as a yellow solid. MS (ESI) m/z: 764.1 [M+1]⁺.

Step 2: Preparation of (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(4-methylthiazol-5-yl)phenyl]ethyl]pyrrolidine-2-carboxamide

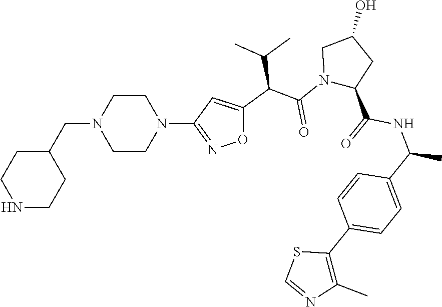 [SMILES: Cc1ncsc1-c1ccc([C@H](C)NC(=O)[C@@H]2C[C@@H](O)CN2C(=O)[C@@H](c2cc(N3CCN(CC4CCNCC4)CC3)no2)C(C)C)cc1]

To a mixture of 4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]piperidine-1-carboxylate (470 mg, 0.6 mmol) in CH₂Cl₂ (4 mL) was added 4 M HCl in dioxane (2 mL) at 25° C. The mixture was stirred at 25° C. for 30 min, then concentrated in vacuum. The residue was re-dissolved in tetrahydrofuran and adjusted the pH to 10 by triethylamine, filtered and concentrated to afford (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(4-methylthiazol-5-yl)phenyl]ethyl]pyrrolidine-2-carboxamide (349 mg, crude) as a yellow solid, which was used directly in the next step. MS (ESI) m/z: 664.4 [M+1]⁺.

Step 3: Preparation of tert-butyl 3-[8-fluoro-7-[7-fluoro-3-hydroxy-8-(2-triisopropylsilylethynyl)-1-naphthyl]-2-[2-[[4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate

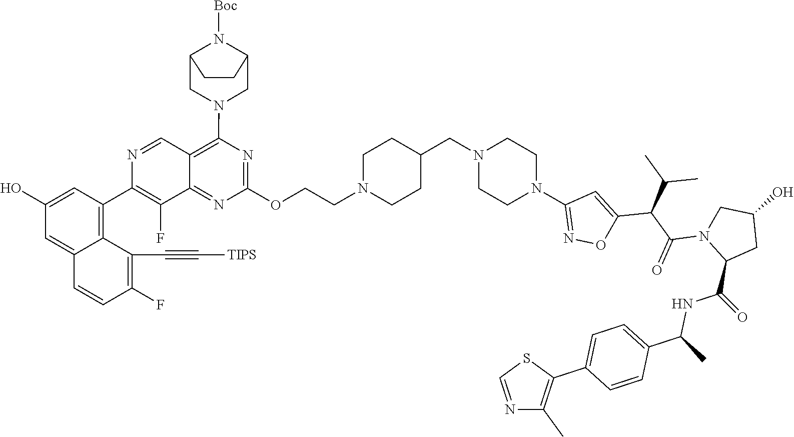

To a mixture of (2S,4R)-4-hydroxy-1-[(2R)-3-methyl-2-[3-[4-(4-piperidylmethyl) piperazin-1-yl]isoxazol-5-yl]butanoyl]-N-[(1S)-1-[4-(4-methylthiazol-5-yl)phenyl]ethyl]pyrrolidine-2-carboxamide (339 mg, 0.5 mmol) and tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate (352 mg, 0.5 mmol) in CH₂Cl₂ (4 mL) and i-PrOH (4 mL) were added acetic acid (106 uL, 1.9 mmol) and 2-methylpyridine borane (248 mg, 2.3 mmol) at 25° C. The mixture was stirred at 25° C. for h, then concentrated in vacuum. The pH of the residue was adjusted to 10 by trimethylamine and purified by column chromatography (0-5% then up to 10% methanol (1N NH₃ as additive) in dichloromethane) to afford tert-butyl 3-[8-fluoro-7-[7-fluoro-3-hydroxy-8-(2-triisopropylsilylethynyl)-1-naphthyl]-2-[2-[[4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (478 mg, 73%) as a yellow solid. MS (ESI) m/z: 1406.7 [M+1]⁺.

Step 4: Preparation of tert-butyl 3-[7-(8-ethynyl-7-fluoro-3-hydroxy-1-naphthyl)-8-fluoro-2-[2-[[4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate

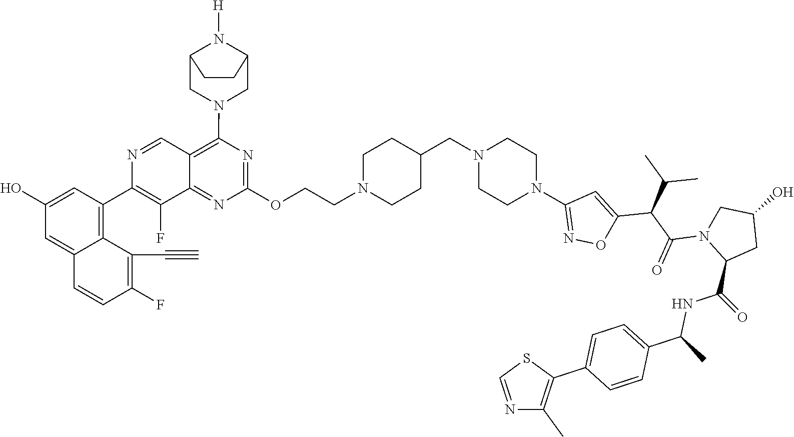 [SMILES: C#Cc1c(F)ccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a solution of tert-butyl 3-[8-fluoro-7-[7-fluoro-3-hydroxy-8-(2-triisopropylsilylethynyl)-1-naphthyl]-2-[2-[[4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (478 mg, 0.3 mmol) in THF (6 mL) was added TBAF (1 M, 1 mL) at 25° C. The mixture was stirred at 25° C. for 72 h, then concentrated in vacuum. The residue was dissolved with ethyl acetate (120 mL) and washed with water (10 mL×6), brine (20 mL), dried with anhydrous Na₂SO₄, filtered, and concentrated to afford tert-butyl 3-[7-(8-ethynyl-7-fluoro-3-hydroxy-1-naphthyl)-8-fluoro-2-[2-[[4-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (422 mg, crude) as a yellow solid, which was used directly in the next step. MS (ESI) m/z: 625.5 [M/2+1]⁺.

Step 5: Preparation of (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

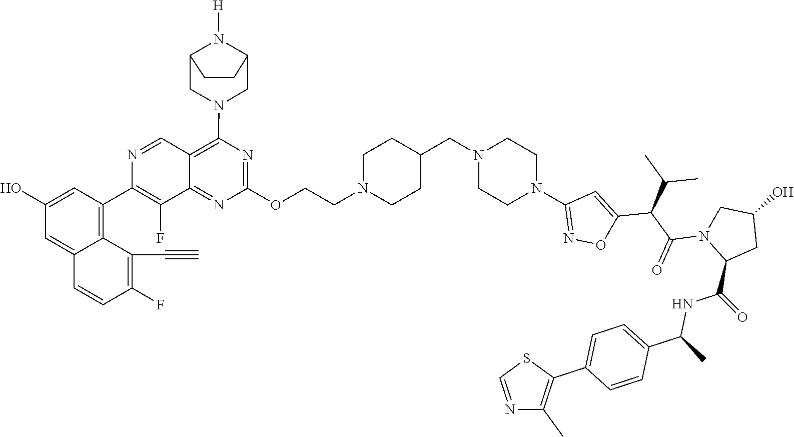 [SMILES: C#Cc1c(F)ccc2cc(O)cc(-c3ncc4c(N5CC6CCC(C5)N6)nc(OCCN5CCC(CN6CCN(c7cc([C@H](C(=O)N8C[C@H](O)C[C@H]8C(=O)N[C@@H](C)c8ccc(-c9scnc9C)cc8)C(C)C)on7)CC6)CC5)nc4c3F)c12]

To a mixture of tert-butyl 3-[7-(8-ethynyl-7-fluoro-3-hydroxy-1-naphthyl)-8-fluoro-2-[2-[[4-[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazin-1-yl]methyl]-1-piperidyl]ethoxy]pyrido[4,3-d]pyrimidin-4-yl]-3,8-diazabicyclo[3.2.1]octane-8-carboxylate (412 mg, 0.3 mmol) in CH₂Cl₂ (4 mL) was added 4 M HCl in dioxane (2 mL). The mixture was stirred at 25° C. for 30 min, then concentrated in vacuum. The pH of the residue was adjusted to 10, suspended in tetrahydrofuran and filtered, concentrated. The residue was purified by preparative HPLC (column: Phenomenex C18 75*30 mm*3 um; mobile phase: [water (NH₃H₂O+NH₄HCO₃)-acetonitrile]; B %: 28%-68%, 36 min) to afford (2S,4R)-1-[(2R)-2-[3-[4-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-piperidyl]methyl]piperazin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (124.5 mg, 31%) as a yellow solid. MS (ESI) m/z: 1149.6 [M+1]⁺; 1H NMR (400 MHz, CD₃OD) δ 9.01 (s, 1H), 8.88 (s, 1H), 7.86 (dd, J=5.6, 9.2 Hz, 1H), 7.46-7.38 (m, 4H), 7.36-7.29 (m, 2H), 7.21 (d, J=2.4 Hz, 1H), 6.12-6.02 (m, 1H), 5.03 (q, J=7.2 Hz, 1H), 4.68-4.55 (m, 5H), 4.51 (t, J=8.0 Hz, 1H), 4.46-4.39 (m, 1H), 3.84 (dd, J=4.2, 10.8 Hz, 1H), 3.74-3.58 (m, 6H), 3.38 (s, 1H), 3.26-3.21 (m, 4H), 3.14-3.07 (m, 2H), 2.88 (t, J=5.6 Hz, 2H), 2.52-2.46 (m, 7H), 2.40-2.31 (m, 1H), 2.25-2.17 (m, 4H), 1.96 (ddd, J=4.8, 8.4, 13.2 Hz, 1H), 1.89-1.77 (m, 6H), 1.68-1.60 (m, 1H), 1.59-1.51 (m, 3H), 1.32-1.24 (m, 2H), 1.05 (d, J=6.4 Hz, 3H), 0.93-0.86 (m, 3H).

Exemplary Synthesis of (2S,4R)-1-[(2S)-2-(3-{[(3S,7aR)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 207)

Step 1: Preparation of (2S,4R)-1-[2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-[tert-butyl(diphenyl)silyl]oxy-N-[(1S)-1-[4-(4-methylthiazol-5-yl)phenyl]ethyl]pyrrolidine-2-carboxamide

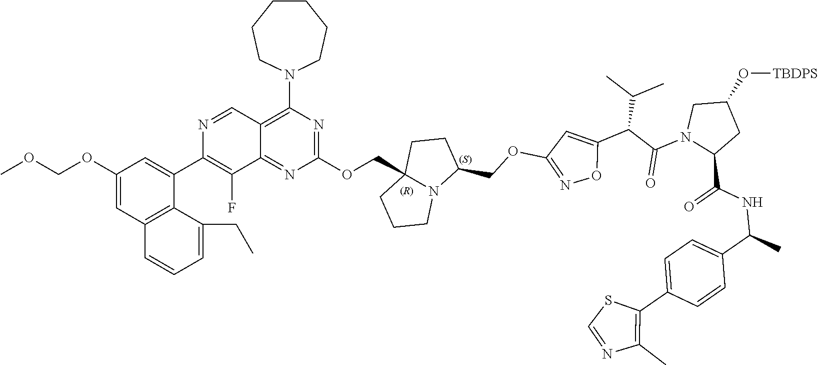

To a solution of (2S,4R)-4-[tert-butyl(diphenyl)silyl]oxy-1-[2-[3-[[(3S,8R)-8-(hydroxymethyl)-1,2,3,5,6,7-hexahydropyrrolizin-3-yl]methoxy]isoxazol-5-yl]-3-methyl-butanoyl]-N-[(1S)-1-[4-(4-methylthiazol-5-yl)phenyl]ethyl]pyrrolidine-2-carboxamide (600 mg, 0.7 mmol), 4 A molecular sieves (1.0 g) and 4-(azepan-1-yl)-7-[8-ethyl-3-(methoxymethoxy)-1-naphthyl]-8-fluoro-2-methylsulfonyl-pyrido[4,3-d]pyrimidine (363 mg, 0.7 mmol) in toluene (12 mL) was added t-BuONa (162 mg, 1.7 mmol) at 0° C. under N₂. The mixture was stirred at 0° C. for 1 h, then adjusted the pH to 8 with 2 M hydrochloric acid and diluted with water (30 mL). The aqueous phase was extracted with ethyl acetate (30 mL×3). The combined organic phase was washed with brine (20 mL), dried with anhydrous Na₂SO₄, filtered and concentrated. The residue was purified by flash column (0-3% then up to 5% methanol in dichloromethane) to afford (2S,4R)-1-[2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-[tert-butyl(diphenyl)silyl]oxy-N-[(1S)-1-[4-(4-methylthiazol-5-yl)phenyl]ethyl]pyrrolidine-2-carboxamide (739 mg, 81%) as a yellow solid. MS (ESI) m/z: 1348.3 [M+1]⁺.

Step 2: Preparation of (2S,4R)-1-[2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

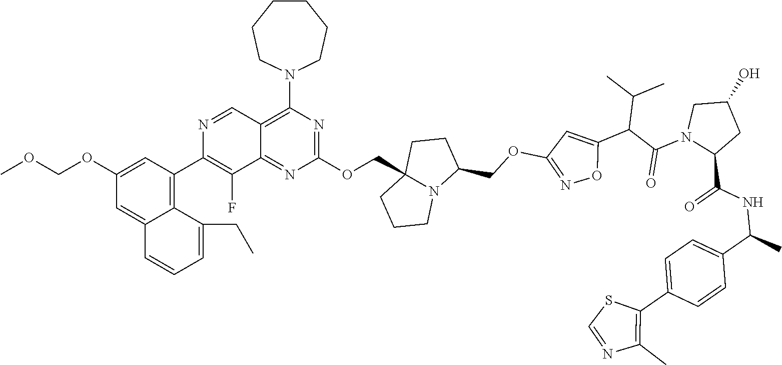 [SMILES: CCc1cccc2cc(OCOC)cc(-c3ncc4c(N5CCCCCC5)nc(OC[C@]56CCCN5[C@H](COc5cc(C(C(=O)N7C[C@H](O)C[C@H]7C(=O)N[C@@H](C)c7ccc(-c8scnc8C)cc7)C(C)C)on5)CC6)nc4c3F)c12]

To a solution of (2S,4R)-1-[2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-[tert-butyl(diphenyl)silyl]oxy-N-[(1S)-1-[4-(4-methylthiazol-5-yl)phenyl]ethyl]pyrrolidine-2-carboxamide (739 mg, 0.5 mmol) in DMF (10 mL) was added CsF (666 mg, 4.4 mmol) at 25° C. The mixture was stirred at 25° C. for 12 h. The reaction mixture was quenched by water (50 mL) and filtered through Celite pad. The filter cake was re-dissolved in ethyl acetate (40 mL), dried over anhydrous Na₂SO₄, filtered and concentrated. The residue was purified by flash column (0-5% then up to 7% methanol in dichloromethane) to afford (2S,4R)-1-[2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (529 mg, 87%) as a yellow solid. MS (ESI) m/z: 1110.4 [M+1]⁺.

Step 3: Preparation of (2S,4R)-1-[(2S)-2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide and (2S,4R)-1-[(2R)-2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

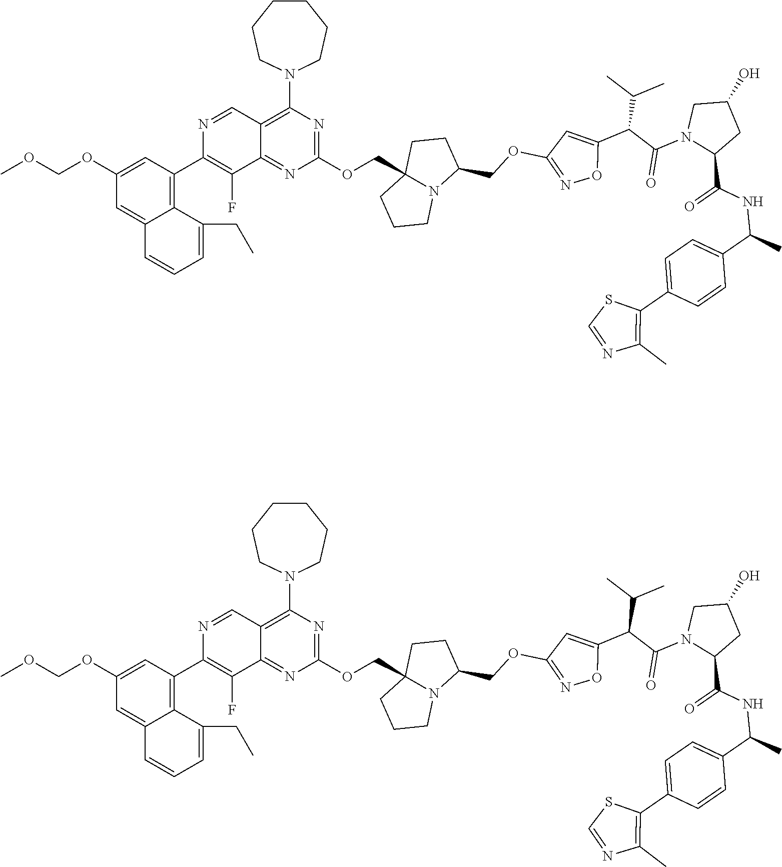

(2S,4R)-1-[2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (480 mg) was separated by chiral SFC (column: DAICEL CHIRALPAK IC (250 mm*30 mm, 10 um); mobile phase: [isopropanol-acetonitrile]; B %: 35%-35%, 15 min). Fraction 1 (t_{R}=5.859 min) was obtained as (2S,4R)-1-[(2S)-2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (260 mg, 54%) as a white solid. MS (ESI) m/z: 1110.3 [M+1]⁺. Fraction 2 (t_{R}=7.529 min) was obtained as (2S,4R)-1-[(2R)-2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (208 mg, 43%) as a white solid. MS (ESI) m/z: 555.8 [M/2+1]⁺.

Step 4: Preparation of (2S,4R)-1-[(2R)-2-[3-[[(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

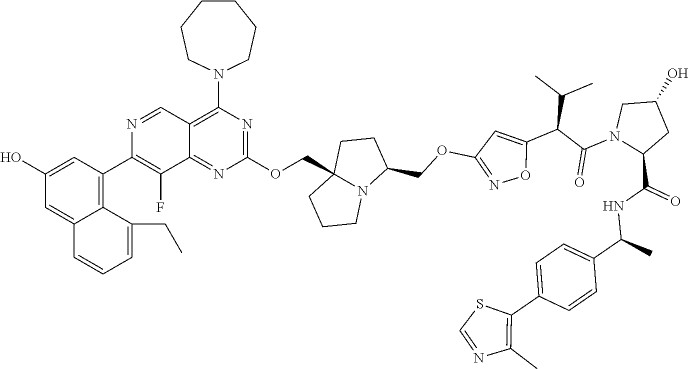 [SMILES: CCc1cccc2cc(O)cc(-c3ncc4c(N5CCCCCC5)nc(OC[C@]56CCCN5[C@H](COc5cc([C@H](C(=O)N7C[C@H](O)C[C@H]7C(=O)N[C@@H](C)c7ccc(-c8scnc8C)cc7)C(C)C)on5)CC6)nc4c3F)c12]

To a mixture of (2S,4R)-1-[(2R)-2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (208 mg, 0.2 mmol) in CH₂Cl₂ (4 mL) was added 4 M HCl in dioxane (2 mL) at 25° C. The mixture was stirred at 25° C. for 30 min, then concentrated in vacuum. The residue was re-dissolved with tetrahydrofuran and adjusted to pH 10 by triethylamine, then filtered and concentrated. The residue was purified by preparative HPLC (column: Xtimate C18 150*40 mm*10 um; mobile phase: [water (formic acid)-acetonitrile]; B %: 10%-50%, 36 min) to afford (2S,4R)-1-[(2R)-2-[3-[[(3S,8R)-8-[[4-(azepan-1-yl)-7-(8-ethyl-3-hydroxy-1-naphthyl)-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (106.8 mg, 51%, 0.38 formic acid salt) as a white solid. MS (ESI) m/z: 1066.3 [M+1]⁺; 1H NMR (400 MHz, CD₃OD) δ 9.12 (d, J=3.2 Hz, 1H), 8.91-8.78 (m, 1H), 8.48 (s, 1H), 7.62 (d, J=8.0 Hz, 1H), 7.48-7.31 (m, 5H), 7.28 (d, J=2.4 Hz, 1H), 7.14 (d, J=6.4 Hz, 1H), 7.07-6.97 (m, 1H), 6.11-5.93 (m, 1H), 4.96 (q, J=7.2 Hz, 1H), 4.71-4.52 (m, 2H), 4.51-4.37 (m, 3H), 4.37-4.25 (m, 2H), 4.09 (t, J=5.6 Hz, 4H), 3.81-3.72 (m, 1H), 3.72-3.65 (m, 1H), 3.65-3.58 (m, 1H), 3.58-3.44 (m, 1H), 3.24-3.09 (m, 1H), 2.50-2.44 (m, 3H), 2.39-2.14 (m, 6H), 2.03 (d, J=4.4 Hz, 8H), 1.97-1.84 (m, 3H), 1.69 (s, 4H), 1.59-1.42 (m, 3H), 1.08-0.93 (m, 3H), 0.92-0.79 (m, 6H).

Exemplary Synthesis of (2S,4R)-1-[(2R)-2-(3-{[(3S,7aR)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 208)

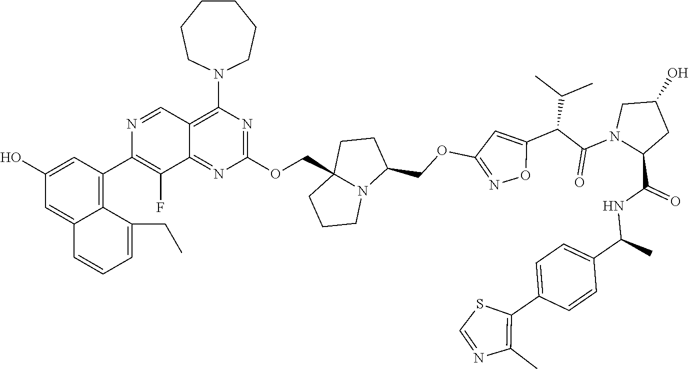 [SMILES: CCc1cccc2cc(O)cc(-c3ncc4c(N5CCCCCC5)nc(OC[C@]56CCCN5[C@H](COc5cc([C@@H](C(=O)N7C[C@H](O)C[C@H]7C(=O)N[C@@H](C)c7ccc(-c8scnc8C)cc7)C(C)C)on5)CC6)nc4c3F)c12]

The title compound was prepared analogously to (2S,4R)-1-[(2S)-2-(3-{[(3S,7aR)-7a-({[4-(azepan-1-yl)-7-(8-ethyl-3-hydroxynaphthalen-1-yl)-8-fluoropyrido[4,3-d]pyrimidin-2-yl]oxy}methyl)-hexahydro-1H-pyrrolizin-3-yl]methoxy}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide, starting from (2S,4R)-1-[(2S)-2-[3-[[(3S,8R)-8-[[4-(azepan-1-yl)-7-[8-ethyl-3-(methoxymethoxy)-1-naphthyl]-8-fluoro-pyrido[4,3-d]pyrimidin-2-yl]oxymethyl]-1,2,3,5,6,7-hexahydropyrrolizin-3-yl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide. (formic acid salt, white solid). MS (ESI) m/z: 1066.9 [M+1]⁺; 1H NMR (400 MHz, CD₃OD) δ 9.22-9.12 (m, 1H), 8.88 (s, 1H), 8.43 (s, 1H), 7.67-7.59 (m, 1H), 7.47-7.32 (m, 5H), 7.31-7.25 (m, 1H), 7.19-7.13 (m, 1H), 7.06-6.89 (m, 1H), 6.02-5.78 (m, 1H), 5.02 (dd, J=2.4, 6.8 Hz, 1H), 4.63-4.52 (m, 3H), 4.50-4.35 (m, 4H), 4.20-4.07 (m, 4H), 3.82-3.74 (m, 1H), 3.73-3.61 (m, 2H), 3.60-3.51 (m, 1H), 3.49-3.40 (m, 1H), 2.49-2.44 (m, 3H), 2.39-2.23 (m, 4H), 2.20-1.98 (m, 12H), 1.96-1.85 (m, 1H), 1.72 (s, 4H), 1.58-1.46 (m, 3H), 1.02 (t, J=6.4 Hz, 3H), 0.93-0.80 (m, 6H).

Exemplary Synthesis of (2S,4R)-1-[(2R)-2-{3-[(2S)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-2-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 209)

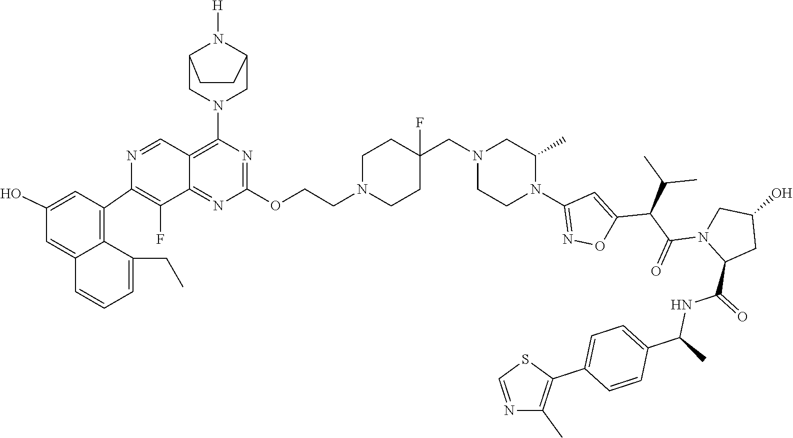 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)[C@@H](C)C6)CC5)nc4c3F)c12]

The title compound was prepared analogously to (2S,4R)-1-[(2S)-2-{3-[(2S)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-2-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide, starting from (2S,4R)-4-hydroxy-1-[(2R)-3-methyl-2-[3-[(2S)-2-methylpiperazin-1-yl]isoxazol-5-yl]butanoyl]-N-[(1S)-1-[4-(4-methylthiazol-5-yl)phenyl]ethyl]pyrrolidine-2-carboxamide and tert-butyl 4-fluoro-4-formyl-piperidine-1-carboxylate. (formic acid salt, white solid). MS (ESI) m/z: 1168.0 [M+H]⁺; ¹H NMR (400 MHz, CD₃OD) δ 9.06 (s, 1H), 8.88 (s, 1H), 8.48 (s, 1H), 7.64 (d, J=8.4 Hz, 1H), 7.48-7.33 (m, 5H), 7.30 (d, J=2.4 Hz, 1H), 7.17 (d, J=7.2 Hz, 1H), 7.01 (d, J=2.4 Hz, 1H), 6.07-5.96 (m, 1H), 5.04 (d, J=7.2 Hz, 1H), 4.79-4.65 (m, 5H), 4.63-4.39 (m, 4H), 3.93-3.72 (m, 6H), 3.67-3.55 (m, 2H), 3.25-3.17 (m, 1H), 3.03 (s, 4H), 2.91 (d, J=11.2 Hz, 1H), 2.81 (d, J=11.6 Hz, 1H), 2.68 (d, J=3.6 Hz, 1H), 2.64 (d, J=2.4 Hz, 1H), 2.56-2.41 (m, 6H), 2.40-2.17 (m, 5H), 2.07-1.90 (m, 7H), 1.60-1.48 (m, 3H), 1.28-1.19 (m, 3H), 1.05 (d, J=6.4 Hz, 3H), 0.95-0.85 (m, 6H).

Exemplary Synthesis of (2S,4R)-1-[(2S)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]piperidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 210)

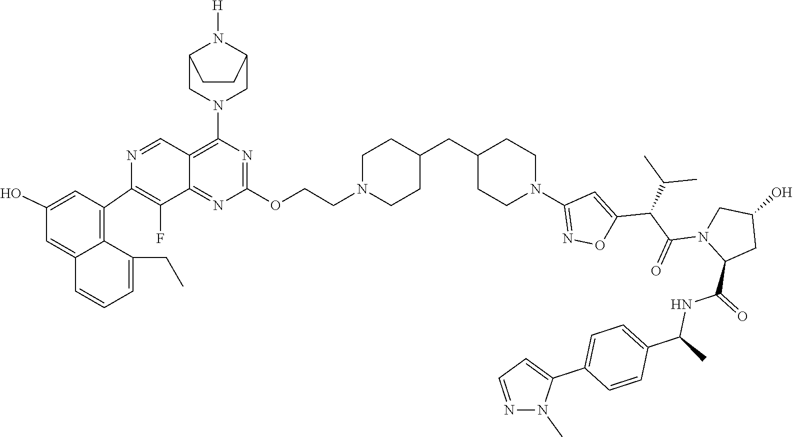 [SMILES: CCc1cccc2cc(O)cc(-c3ncc4c(N5CC6CCC(C5)N6)nc(OCCN5CCC(CC6CCN(c7cc([C@@H](C(=O)N8C[C@H](O)C[C@H]8C(=O)N[C@@H](C)c8ccc(-c9ccnn9C)cc8)C(C)C)on7)CC6)CC5)nc4c3F)c12]

The title compound was prepared analogously to (2S,4R)-1-[(2R)-2-(3-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]piperidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide, starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[4-(4-piperidylmethyl)-1-piperidyl]isoxazol-5-yl]butanoyl]-N-[(1S)-1-[4-(2-methylpyrazol-3-yl)phenyl]ethyl]pyrrolidine-2-carboxamide. (white solid). MS (ESI) m/z: 1118.0 [M+H]⁺; 1H NMR (400 MHz, CD₃OD) δ 9.04 (s, 1H), 7.62 (d, J=8.0 Hz, 1H), 7.49-7.32 (m, 6H), 7.28 (d, J=2.8 Hz, 1H), 7.16 (d, J=7.2 Hz, 1H), 7.01 (d, J=2.4 Hz, 1H), 6.33 (d, J=2.0 Hz, 1H), 6.11 (s, 1H), 5.00 (s, 1H), 4.68-4.53 (m, 6H), 4.43 (s, 1H), 4.18-4.18 (m, 1H), 3.88-3.83 (m, 3H), 3.76-3.58 (m, 10H), 3.10 (d, J=11.2 Hz, 2H), 2.88 (t, J=5.6 Hz, 2H), 2.82-2.72 (m, 2H), 2.38-2.14 (m, 6H), 1.90-1.78 (m, 4H), 1.76-1.66 (m, 3H), 1.61-1.45 (m, 5H), 1.23-1.12 (m, 5H), 1.05 (d, J=6.8 Hz, 3H), 0.95-0.87 (m, 6H).

Exemplary Synthesis of (2S,4R)-1-[(2R)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 212)

Step 1: Preparation of tert-butyl 3-[2-[2-[tert-butyl(dimethyl)silyl]oxyethoxy]-7-(8-ethyl-3-tetrahydropyran-2-yloxy-1-naphthyl)-8-fluoro-pyrido[4,3-d]pyrimidin-4-yl]-3,8-diaza bicyclo[3.2.1]octane-8-carboxylate

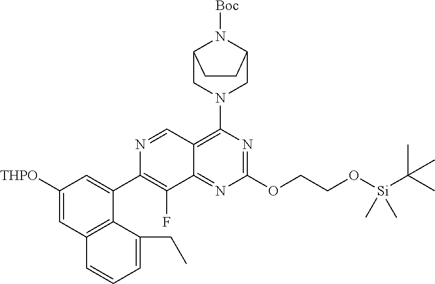

A mixture of tert-butyl 3-[2-[2-[tert-butyl(dimethyl)silyl]oxyethoxy]-7-chloro-8-fluoro-pyrido[4,3-d]pyrimidin-4-yl]-3,8-diazabicyclo[3.2.1]octane-8-carboxylate (14 g, 24.64 mmol, 1 eq), 2-(8-ethyl-3-tetrahydropyran-2-yloxy-1-naphthyl)-4,4,5,5-tetramethyl-1,3,2-dioxaborolane (10.36 g, 27 mmol), potassium phosphate (15.69 g, 74 mmol) and [2-(2-aminophenyl)phenyl]palladium (1+); bis(1-adamantyl)-butyl-phosphane; methanesulfonate (1.79 g, 2.5 mmol) in dioxane (150 mL) and water (20 mL) was degassed and purged with nitrogen for 3 times, the mixture was stirred at 90° C. for 12 h under nitrogen atmosphere. The reaction mixture was quenched with water (200 mL), extracted with ethyl acetate (200 mL×3). The combined organic phase was washed with brine (200 mL), dried with anhydrous sodium sulfate, filtered, and concentrated in vacuo. The residue was purified by silica gel column chromatography (petroleum ether/ethyl acetate=20/1 to 1/1) to afford tert-butyl 3-[2-[2-[tert-butyl(dimethyl)silyl]oxyethoxy]-7-(8-ethyl-3-tetrahydropyran-2-yloxy-1-naphthyl)-8-fluoro-pyrido[4,3-d]pyrimidin-4-yl]-3,8-diazabicyclo[3.2.1]octane-8-carboxylate (16 g, 82%) as a yellow solid. MS (ESI) m/z: 788.5 [M+1]⁺.

Step 2: Preparation of tert-butyl 3-[7-(8-ethyl-3-tetrahydropyran-2-yloxy-1-naphthyl)-8-fluoro-2-(2-hydroxyethoxy)pyrido[4,3-d]pyrimidin-4-yl]-3,8-diazabicyclo[3.2.1]octane-8-carboxylate

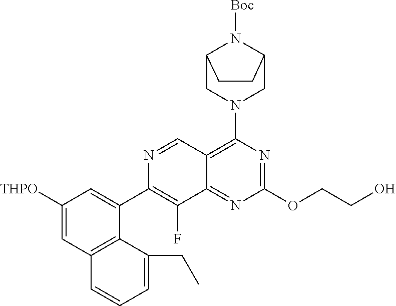

To a solution of tert-butyl 3-[2-[2-[tert-butyl(dimethyl)silyl]oxyethoxy]-7-(8-ethyl-3-tetrahydropyran-2-yloxy-1-naphthyl)-8-fluoro-pyrido[4,3-d]pyrimidin-4-yl]-3,8-diazabicyclo[3.2.1]octane-8-carboxylate (16 g, 20 mmol) in N,N-dimethylformamide (160 mL) was added cesium fluoride (77.1 g, 507 mmol), then the mixture was stirred for 12 h at 25° C. The reaction mixture was quenched with water (200 mL), extracted with ethyl acetate (200 mL×3). The combined organic phase was washed with brine (200 mL), dried with anhydrous sodium sulfate, filtered, and concentrated in vacuo. The residue was purified by prep-HPLC (column: UniSil 10-120 C18 70×250 mm; mobile phase: [water (formic acid)-acetonitrile]; B %: 45%-75%, 35 min) to afford tert-butyl 3-[7-(8-ethyl-3-tetrahydropyran-2-yloxy-1-naphthyl)-8-fluoro-2-(2-hydroxyethoxy)pyrido[4,3-d]pyrimidin-4-yl]-3,8-diazabicyclo[3.2.1]octane-8-carboxylate (9 g, 65%) as a yellow solid. MS (ESI) m/z: 674.4 [M+1]⁺.

Step 3: Preparation of tert-butyl 3-[7-(8-ethyl-3-tetrahydropyran-2-yloxy-1-naphthyl)-8-fluoro-2-(2-oxoethoxy)pyrido[4,3-d]pyrimidin-4-yl]-3,8-diazabicyclo[3.2.1]octane-8-carboxylate

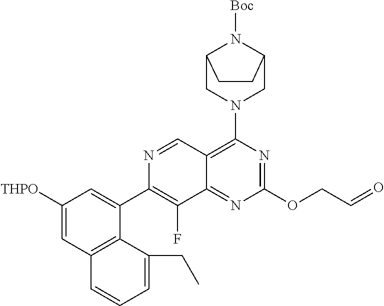

To a solution of dimethyl sulfoxide (1.45 g, 18 mmol, 1.5 mL) in dichloromethane (20 mL) was added dropwise to a solution of oxalyl chloride (1.41 g, 11 mmol, 1 mL) in dichloromethane (20 mL) at −78° C. The mixture was stirred at same temperature for 30 min, then a solution of tert-butyl 3-[7-(8-ethyl-3-tetrahydropyran-2-yloxy-1-naphthyl)-8-fluoro-2-(2-hydroxyethoxy)pyrido[4,3-d]pyrimidin-4-yl]-3,8-diazabicyclo[3.2.1]octane-8-carboxylate (5 g, 7 mmol) in dichloromethane (20 mL) was added dropwise. The mixture was stirred at −78° C. for 30 min before the addition of triethylamine (3.75 g, 37 mmol, 5.2 mL). The resulting mixture was stirred at the same temperature for 30 min and warmed to 25° C. for 1 h. The reaction mixture was quenched with water (50 mL), extracted with dichloromethane (50 mL×3). The combined organic phase was washed with brine (50 mL), dried with anhydrous sodium sulfate, filtered, and concentrated in vacuo. The residue was purified by silica gel column chromatography (petroleum ether/ethyl acetate=10/1 to 1/1) to afford tert-butyl 3-[7-(8-ethyl-3-tetrahydropyran-2-yloxy-1-naphthyl)-8-fluoro-2-(2-oxoethoxy)pyrido[4,3-d]pyrimidin-4-yl]-3,8-diazabicyclo[3.2.1]octane-8-carboxylate (4.5 g, 90%) as a yellow solid. MS (ESI) m/z: 672.3 [M+1]⁺.

Step 4: Preparation of tert-butyl 3-[7-(8-ethyl-3-tetrahydropyran-2-yloxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate

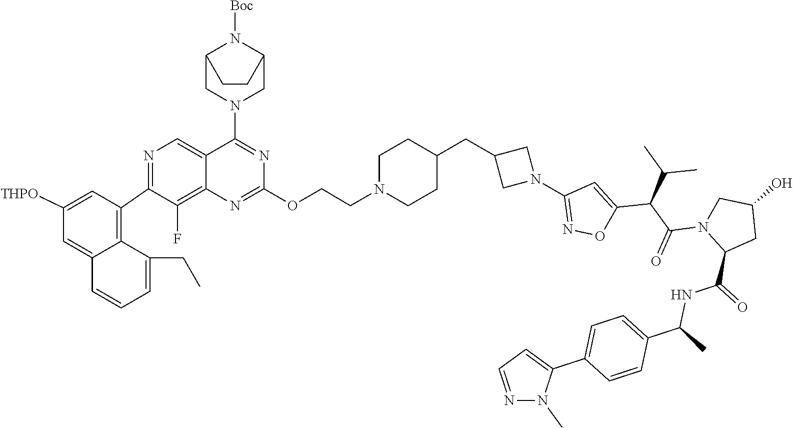

To a solution of (2S,4R)-4-hydroxy-1-[(2R)-3-methyl-2-[3-[[3-(4-piperidylmethyl) azetidin-1-yl]isoxazol-5-yl]butanoyl]-N-[(1S)-1-[4-(2-methylpyrazol-3-yl)phenyl]ethyl]pyrrolidine-2-carboxamide trifluoroacetate (1.7 g, 2 mmol) in dichloromethane (15 mL) and isopropanol (15 mL) was added N,N-diisopropylethylamine (300 mg, 2 mmol, 0.4 mL), then tert-butyl 3-[7-(8-ethyl-3-tetrahydropyran-2-yloxy-1-naphthyl)-8-fluoro-2-(2-oxoethoxy)pyrido[4,3-d]pyrimidin-4-yl]-3,8-diazabicyclo[3.2.1]octane-8-carboxylate (1.87 g, 2.8 mmol) was added. The solution was stirred at 25° C. for 10 min. To the solution was added sodium triacetoxyborohydride (1.48 g, 7 mmol), the reaction solution was stirred at 25° C. for 0.5 h, then concentrated in vacuum. The residue was purified by silica gel column chromatography (petroleum ether/ethyl acetate=1/1 to dichloromethane/methanol=10/1) to afford tert-butyl 3-[7-(8-ethyl-3-tetrahydropyran-2-yloxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate (2.17 g, 73%) as a white solid. MS (ESI) m/z: 1273.8 [M+1]⁺; 1H NMR (400 MHz, MeOD-d₄) δ 9.15 (s, 1H), 8.43 (d, J=7.6 Hz, 1H), 7.78 (d, J=8.0 Hz, 1H), 7.67-7.55 (m, 1H), 7.51-7.41 (m, 4H), 7.38 (d, J=8.4 Hz, 2H), 7.23 (d, J=7.2 Hz, 1H), 7.15 (dd, J=2.8, 4.8 Hz, 1H), 6.41-6.33 (m, 1H), 5.82 (s, 1H), 5.70-5.61 (m, 1H), 5.20-5.07 (m, 1H), 4.93 (t, J=7.2 Hz, 1H), 4.59 (s, 3H), 4.51-4.43 (m, 1H), 4.36 (t, J=7.6 Hz, 1H), 4.28 (s, 3H), 3.94 (t, J=6.8 Hz, 2H), 3.84 (s, 3H), 3.81-3.66 (m, 3H), 3.66-3.55 (m, 6H), 3.44 (dd, J=3.6, 6.0 Hz, 4H), 3.37-3.30 (m, 2H), 3.13-3.05 (m, 3H), 2.83-2.71 (m, 1H), 2.36-2.14 (m, 4H), 2.06-2.00 (m, 1H), 1.88-1.75 (m, 5H), 1.72-1.59 (m, 6H), 1.53 (s, 3H), 1.46 (s, 9H), 1.38 (d, J=7.2 Hz, 3H), 0.99-0.91 (m, 3H), 0.83 (t, J=7.6 Hz, 4H), 0.77 (s, 2H).

Step 5: Preparation of (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide

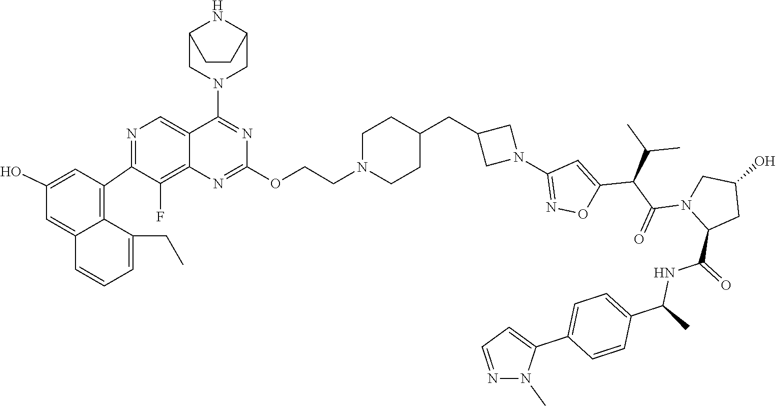 [SMILES: CCc1cccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9ccnn9C)cc8)C(C)C)on7)C6)CC5)nc4c3F)c12]

To a solution of tert-butyl 3-[7-(8-ethyl-3-tetrahydropyran-2-yloxy-1-naphthyl)-8-fluoro-2-[2-[[4-[[1-[5-[(1R)-1-[(2S,4R)-4-hydroxy-2-[[(1S)-1-[4-(2-methylpyrazol-3-yl)phenyl]ethyl]carbamoyl]pyrrolidine-1-carbonyl]-2-methyl-propyl]isoxazol-3-yl]azetidin-3-yl]methyl]-1-piperidyl]ethoxy]pyrido[4,3-d]pyrimidin-4-yl]-3,8-diazabicyclo[3.2.1]octane-8-carboxylate (2.2 g, 1.7 mmol) in dichloromethane (30 mL) was added trifluoroacetic acid (15.4 g, 135 mmol, 10 mL), the reaction solution was stirred at 25° C. for 20 min, then concentrated in vacuum. The residue was purified by prep-HPLC (column: Phenomenex luna C18 250*50 mm*10 um; mobile phase: [water (formic acid)-acetonitrile]; B %: 23%-53%, 10 min). The pH of the crude solution was adjusted to 8-9 by sodium bicarbonate solid. The aqueous layer was extracted with dichloromethane/isopropanol (v/v=10/1, 200 mL×3). The combined organic layer was washed with water (200 mL×3). The organic layer was dried over anhydrous sodium sulfate, filtered, and concentrated under vacuum. The residue was azeotroped with acetonitrile (200 mL) for three times. The product was dissolved with pure water (300 mL) and lyophilized to afford (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]isoxazol-5-yl]-3-methyl-butanoyl]-4-hydroxy-N-[(1S)-1-[4-(2-methylpyrazol-3-yl)phenyl]ethyl]pyrrolidine-2-carboxamide (1156.9 mg, 50%) as an off-white solid. MS (ESI) m/z: 1089.5 [M+1]⁺; 1H NMR (400 MHz, MeOD-d₄) δ 9.04 (s, 1H), 7.62 (d, J=8.4 Hz, 1H), 7.48 (d, J=2.0 Hz, 1H), 7.47-7.40 (m, 4H), 7.39-7.32 (m, 1H), 7.28 (d, J=2.8 Hz, 1H), 7.15 (d, J=7.2 Hz, 1H), 7.01 (d, J=2.8 Hz, 1H), 6.34 (d, J=2.0 Hz, 1H), 5.87-5.77 (m, 1H), 5.04 (d, J=7.2 Hz, 1H), 4.67-4.57 (m, 4H), 4.51 (t, J=8.4 Hz, 1H), 4.43 (s, 1H), 4.03 (t, J=7.6 Hz, 2H), 3.85 (s, 3H), 3.84-3.79 (m, 1H), 3.77-3.69 (m, 2H), 3.68-3.62 (m, 3H), 3.61 (s, 1H), 3.58-3.45 (m, 3H), 3.07 (d, J=11.6 Hz, 2H), 2.92-2.82 (m, 3H), 2.40-2.23 (m, 3H), 2.21-2.10 (m, 3H), 1.95 (s, 1H), 1.89-1.82 (m, 2H), 1.81-1.74 (m, 2H), 1.67 (d, J=10.0 Hz, 2H), 1.62-1.57 (m, 2H), 1.52 (d, J=7.2 Hz, 3H), 1.35-1.23 (m, 3H), 1.04 (d, J=6.4 Hz, 3H), 0.93-0.85 (m, 6H).

Exemplary Synthesis of (2S,4R)-1-[(2R)-2-{3-[(3S)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-3-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 214)

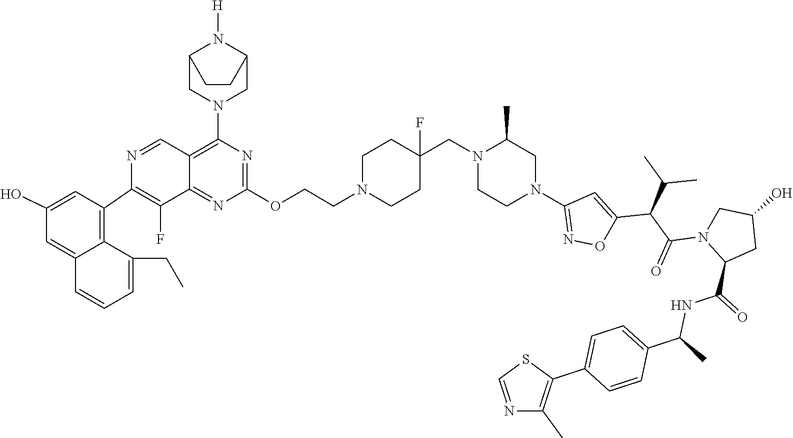 [SMILES: CCc1cccc2cc(O)cc(-c3ncc4c(N5CC6CCC(C5)N6)nc(OCCN5CCC(F)(CN6CCN(c7cc([C@H](C(=O)N8C[C@H](O)C[C@H]8C(=O)N[C@@H](C)c8ccc(-c9scnc9C)cc8)C(C)C)on7)C[C@@H]6C)CC5)nc4c3F)c12]

The title compound was prepared analogously to (2S,4R)-1-[(2S)-2-{3-[(3S)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-3-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide, starting from (2S,4R)-4-hydroxy-1-[(2R)-3-methyl-2-[3-[(3S)-3-methylpiperazin-1-yl]isoxazol-5-yl]butanoyl]-N-[(1S)-1-[4-(4-methylthiazol-5-yl)phenyl]ethyl]pyrrolidine-2-carboxamide and tert-butyl 4-fluoro-4-formyl-piperidine-1-carboxylate. (formic acid salt, white solid). MS (ESI) m/z: 1168.0 [M+1]⁺; 1H NMR (400 MHz, CD₃OD) δ 9.07 (s, 1H), 8.87 (s, 1H), 8.47 (s, 1H), 7.63 (d, J=8.0 Hz, 1H), 7.47-7.32 (m, 5H), 7.29 (d, J=2.4 Hz, 1H), 7.16 (d, J=7.2 Hz, 1H), 7.00 (d, J=2.8 Hz, 1H), 6.12-6.02 (m, 1H), 5.07-4.98 (m, 1H), 4.79-4.65 (m, 5H), 4.51 (t, J=8.4 Hz, 1H), 4.44 (d, J=1.6 Hz, 1H), 3.98 (d, J=9.2 Hz, 2H), 3.90-3.76 (m, 3H), 3.66-3.48 (m, 2H), 3.34 (d, J=1.6 Hz, 1H), 3.17-3.02 (m, 6H), 2.85-2.68 (m, 4H), 2.66-2.58 (m, 1H), 2.52-2.42 (m, 5H), 2.41-2.22 (m, 4H), 2.18-2.10 (m, 1H), 2.05-1.92 (m, 6H), 1.88-1.72 (m, 2H), 1.58 (d, J=7.2 Hz, 1H), 1.52 (d, J=7.2 Hz, 2H), 1.05 (dd, J=1.6, 6.4 Hz, 6H), 0.93-0.86 (m, 6H).

Exemplary Synthesis of (2S,4R)-1-[(2R)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)oxy]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 215)

Step 1: Preparation of tert-butyl 3-(4-pyridyloxy)azetidine-1-carboxylate

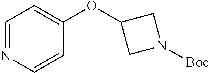 [SMILES: CC(C)(C)OC(=O)N1CC(Oc2ccncc2)C1]

To a solution of NaH (2.8 g, 69 mmol, 60%) in DMSO (70 mL) was added 4-fluoropyridine hydrochloride (3.1 g, 23 mmol) and the mixture was stirred 25° C. for 30 min. Then a solution of tert-butyl 3-hydroxyazetidine-1-carboxylate (4.0 g, 23 mmol) in DMSO (30 mL) was added dropwise. The mixture was stirred at 25° C. for 16 h. The mixture was quenched by saturated aqueous NH₄Cl (150 mL), extracted with ethyl acetate (120 mL×3). The combined organic phase was washed with brine (30 mL×2), dried over anhydrous sodium sulfate, filtered, and concentrated in vacuum. The residue was purified by flash column (0-74% ethyl acetate in petroleum ether) to afford tert-butyl 3-(4-pyridyloxy)azetidine-1-carboxylate (3.7 g, 59%) as a white solid. MS (ESI) m/z: 251.0 [M+1]⁺; 1H NMR (400 MHz, DMSO-d₆) δ 8.45-8.36 (m, 2H), 6.91-6.83 (m, 2H), 5.10-5.05 (m, 1H), 4.38-4.28 (m, 2H), 3.81-3.79 (m, 2H), 1.38 (s, 9H).

Step 2: Preparation of 4-(azetidin-3-yloxy)pyridine

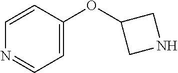 [SMILES: c1cc(OC2CNC2)ccn1]

To a solution of tert-butyl 3-(4-pyridyloxy)azetidine-1-carboxylate (4.1 g, 16 mmol) in CH₂Cl₂ (40 mL) was added TFA (13.0 mL, 175 mmol). The mixture was stirred at 25° C. for 1 h, then concentrated in vacuum to give 4-(azetidin-3-yloxy)pyridine (6.2 g, 97% yield, di-trifluoroacetate salt) as a yellow oil. MS (ESI) m/z: 151.0 [M+1]⁺; 1H NMR (400 MHz, DMSO-d₆) δ 8.83 (d, J=7.2 Hz, 2H), 7.54 (d, J=7.2 Hz, 2H), 5.60-5.30 (m, 1H), 4.55 (s, 2H), 4.16 (s, 2H).

Step 3: Preparation of 2-trimethylsilylethyl 3-(4-pyridyloxy)azetidine-1-carboxylate

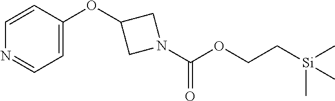 [SMILES: C[Si](C)(C)CCOC(=O)N1CC(Oc2ccncc2)C1]

To a solution of 4-(azetidin-3-yloxy)pyridine di-trifluoroacetate (6.2 g, 16 mmol) in CH₂Cl₂ (60 mL) was added triethylamine (23.0 mL, 164 mmol) and (2,5-dioxopyrrolidin-1-yl) 2-trimethylsilylethyl carbonate (6.4 g, 24 mmol). The mixture was stirred at 25° C. for 1 h. The reaction was diluted with water (40 mL), the organic layer was separated and the aqueous layer was extracted with CH₂Cl₂ (30 mL×2). The combined organic layer was washed with brine (30 mL×2), dried over Na₂SO₄, filtered, and concentrated under reduced pressure. The residue was purified by flash column (0-40% then up to 100% ethyl acetate in petroleum ether) to afford 2-trimethylsilylethyl 3-(4-pyridyloxy)azetidine-1-carboxylate (4.3 g, 88%) as a colorless oil. MS (ESI) m/z: 295.0 [M+1]⁺.

Step 4: Preparation of 2-trimethylsilylethyl 3-(4-piperidyloxy)azetidine-1-carboxylate

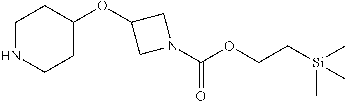 [SMILES: C[Si](C)(C)CCOC(=O)N1CC(OC2CCNCC2)C1]

To a solution of 2-trimethylsilylethyl 3-(4-pyridyloxy)azetidine-1-carboxylate (4.3 g, 15 mmol) in ethanol (150 mL) was added acetic acid (1.7 mL, 29 mmol) and PtO₂ (995 mg, 4 mmol). The mixture was stirred at 70° C. for 48 h under H₂. The reaction mixture was filtered and concentrated under reduced pressure to give 2-trimethylsilylethyl 3-(4-piperidyloxy)azetidine-1-carboxylate (4.3 g, 98%) as a black oil. MS (ESI) m/z: 301.1 [M+1]⁺; ¹H NMR (400 MHz, DMSO-d₆) δ 4.43-4.35 (m, 1H), 4.14-3.99 (m, 4H), 3.71-3.59 (m, 2H), 3.43-3.37 (m, 1H), 2.99-2.86 (m, 2H), 2.70-2.53 (m, 2H), 2.53-2.48 (m, 2H), 1.82-1.78 (m, 1H), 1.60-1.53 (m, 1H), 1.43-1.37 (m, 1H), 0.91 (t, J=16.4 Hz, 2H), 0.01 (s, 9H).

Step 5: Preparation of tert-butyl 4-[[1-(2-trimethylsilylethoxycarbonyl) azetidin-3-yl]oxypiperidine-1-carboxylate

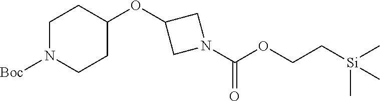 [SMILES: CC(C)(C)OC(=O)N1CCC(OC2CN(C(=O)OCC[Si](C)(C)C)C2)CC1]

To a solution of 2-trimethylsilylethyl 3-(4-piperidyloxy)azetidine-1-carboxylate (4.3 g, 14 mmol) in CH₂Cl₂ (50 mL) was added triethylamine (12 mL, 88 mmol) and Boc₂O (6.7 mL, 29 mmol). The mixture was stirred at 25° C. for 16 h, then concentrated in vacuum. The residue was purified by flash column (0-13% ethyl acetate in petroleum ether) to afford tert-butyl 4-[[1-(2-trimethylsilylethoxycarbonyl) azetidin-3-yl]oxypiperidine-1-carboxylate (4.1 g, 70%) as a yellow oil. MS (ESI) m/z: 423.1 [M+23]⁺; ¹H NMR (400 MHz, CDCl₃) δ 4.39-4.31 (m, 1H), 4.18-4.09 (m, 4H), 3.88-3.85 (m, 2H), 3.82-3.72 (m, 2H), 3.46-3.42 (m, 1H), 3.08-3.01 (m, 2H), 1.78-1.74 (m 2H), 1.55-1.47 (m, 2H), 1.45 (s, 9H), 0.99-0.93 (m, 2H), 0.03 (s, 9H).

Step 6: Preparation of tert-butyl 4-(azetidin-3-yloxy)piperidine-1-carboxylate

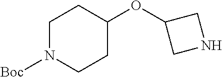 [SMILES: CC(C)(C)OC(=O)N1CCC(OC2CNC2)CC1]

To a solution of tert-butyl 4-[[1-(2-trimethylsilylethoxycarbonyl) azetidin-3-yl]oxypiperidine-1-carboxylate (4.0 g, 10 mmol) in DMA (40 mL) was added CsF (7.6 g, 50 mmol). The mixture was stirred at 90° C. for 1 h. The mixture was diluted with ethyl acetate (80 mL) and filtered. The filtrate was concentrated in vacuum to give tert-butyl 4-(azetidin-3-yloxy)piperidine-1-carboxylate (2.5 g, 98%) as a yellow oil. MS (ESI) m/z: 257.1 [M+1]⁺.

Step 7: Preparation of tert-butyl 4-[[1-[5-(1-methoxycarbonyl-2-methyl-propyl)isoxazol-3-yl]azetidin-3-yl]oxypiperidine-1-carboxylate

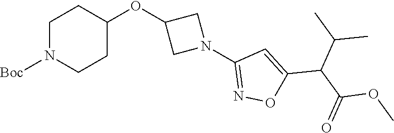 [SMILES: COC(=O)C(c1cc(N2CC(OC3CCN(C(=O)OC(C)(C)C)CC3)C2)no1)C(C)C]

To a solution of tert-butyl 4-(azetidin-3-yloxy)piperidine-1-carboxylate (2.5 g, 9.8 mmol) in DMA (100 mL) was added triethylamine (4.1 mL, 29 mmol) and 4 A MS (7.0 g). The resulting suspension was heated to 130° C., then a solution of methyl 3-methyl-2-[3-(1,1,2,2,3,3,4,4,4-nonafluorobutylsulfonyloxy)isoxazol-5-yl]butanoate (4.7 g, 9.8 mmol) in DMA (30 mL) was added dropwise in 3 h. The mixture was stirred at 130° C. for 16 h. The reaction mixture was diluted with ethyl acetate (400 mL) and filtered. The filtrate was washed with water (50 mL×5), brine (60 mL), dried over anhydrous sodium sulfate, filtered and concentrated in the residue was purified by column chromatography (0-13% then up to 15% tetrahydrofuran in petroleum ether) to afford tert-butyl 4-[[1-[5-(1-methoxycarbonyl-2-methyl-propyl)isoxazol-3-yl]azetidin-3-yl]oxypiperidine-1-carboxylate (2.1 g, 47%) as a yellow oil. MS (ESI) m/z: 438.2 [M+1]⁺; 1H NMR (400 MHz, DMSO-d₆) δ 5.95 (s, 1H), 4.58-4.51 (m, 1H), 4.12-4.05 (m, 2H), 3.69-3.66 (m, 2H), 3.65 (s, 3H), 3.54-3.46 (m, 1H), 2.78 (s, 4H), 2.28-2.25 (m, 1H), 1.80-1.70 (m, 2H), 1.38 (s, 9H), 1.36-1.25 (m, 3H), 0.92 (d, J=6.4 Hz, 3H), 0.83 (d, J=6.8 Hz, 3H).

Step 8: Preparation of 2-[3-[[3-[(1-tert-butoxycarbonyl-4-piperidyl)oxy]azetidin-1-yl]isoxazol-5-yl]-3-methyl-butanoic acid

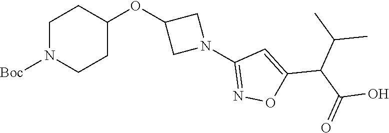 [SMILES: CC(C)C(C(=O)O)c1cc(N2CC(OC3CCN(C(=O)OC(C)(C)C)CC3)C2)no1]

To a solution of tert-butyl 4-[[1-[5-(1-methoxycarbonyl-2-methyl-propyl)isoxazol-3-yl]azetidin-3-yl]oxypiperidine-1-carboxylate (1.0 g, 2.3 mmol) in tetrahydrofuran (10 mL) was added a solution of LiOH·H₂O (336 mg, 8 mmol) in H₂O (5.0 mL). The mixture was stirred at 25° C. for 16 h. The mixture was diluted with saturated aqueous HCl (2 M) to pH 5-6 and extracted with ethyl acetate (30 mL×3). The combined organic phase was washed with brine (20 mL), dried over anhydrous sodium sulfate, filtered, and concentrated in vacuum to give 2-[3-[[3-[(1-tert-butoxycarbonyl-4-piperidyl)oxy]azetidin-1-yl]isoxazol-5-yl]-3-methyl-butanoic acid (800 mg, 78%) as a yellow oil. nMS (ESI) m/z: 424.1 [M+1]⁺.

Step 9: Preparation of tert-butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxypiperidine-1-carboxylate

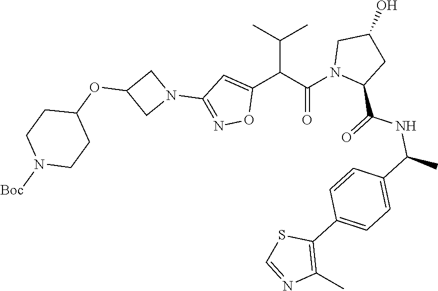 [SMILES: Cc1ncsc1-c1ccc([C@H](C)NC(=O)[C@@H]2C[C@@H](O)CN2C(=O)C(c2cc(N3CC(OC4CCN(C(=O)OC(C)(C)C)CC4)C3)no2)C(C)C)cc1]

To a solution of 2-[3-[[3-[(1-tert-butoxycarbonyl-4-piperidyl)oxy]azetidin-1-yl]isoxazol-5-yl]-3-methyl-butanoic acid (800 mg, 1.9 mmol) and (2S,4R)-4-hydroxy-N-[(1S)-1-[4-(4-methylthiazol-5-yl)phenyl]ethyl]pyrrolidine-2-carboxamide (894 mg, 1.9 mmol) in DMF (15 mL) was added DIEA (1.7 mL, 9.5 mmol) and HATU (790 mg, 2.1 mmol). The mixture was stirred at 25° C. for 1 h. The reaction mixture was diluted with ethyl acetate (70 mL) and washed with water (15 mL×5), brine (20 mL), dried over anhydrous sodium sulfate, filtered, and concentrated in vacuum. The residue was purified by flash column (0-3% methanol in dichloromethane) to afford tert-butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxypiperidine-1-carboxylate (1.0 g, 70%) as a yellow solid. MS (ESI) m/z: 737.4 [M+1]⁺.

Step 10: Preparation of tert-butyl 4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxypiperidine-1-carboxylate and tert-butyl 4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxypiperidine-1-carboxylate

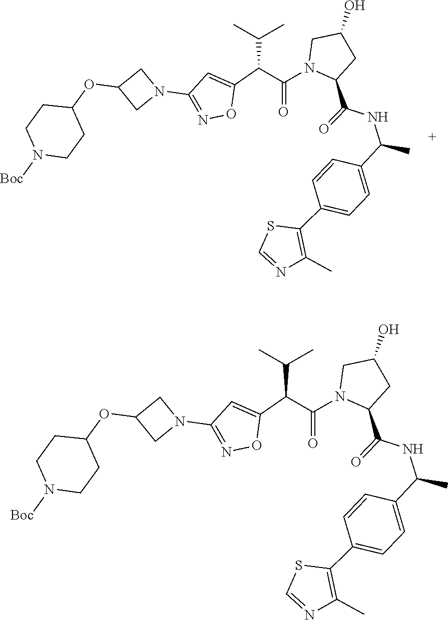 [SMILES: Cc1ncsc1-c1ccc([C@H](C)NC(=O)[C@@H]2C[C@@H](O)CN2C(=O)[C@H](c2cc(N3CC(OC4CCN(C(=O)OC(C)(C)C)CC4)C3)no2)C(C)C)cc1 ; Cc1ncsc1-c1ccc([C@H](C)NC(=O)[C@@H]2C[C@@H](O)CN2C(=O)[C@@H](c2cc(N3CC(OC4CCN(C(=O)OC(C)(C)C)CC4)C3)no2)C(C)C)cc1]

Tert-butyl 4-[[1-[5-[1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxypiperidine-1-carboxylate (1.0 g) was separated by SFC (column: DAICEL CHIRALCEL OD (250 mm*30 mm, 10 um); mobile phase: [0.1% NH₃H₂O/ethanol]; B %: 50%-50%) to afford tert-butyl 4-[[1-[5-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxypiperidine-1-carboxylate (410 mg, 39%) as a white solid. MS (ESI) m/z: 737.3 [M+1]⁺. Tert-butyl 4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxypiperidine-1-carboxylate (375 mg, 36%) was obtained as a white solid. MS (ESI) m/z: 737.4 [M+1]⁺.

Step 11: Preparation of (2S,4R)-4-hydroxy-1-[(2R)-3-methyl-2-[3-[[3-(4-piperidyloxy) azetidin-1-yl]isoxazol-5-yl]butanoyl]-N-[(1S)-1-[4-(4-methylthiazol-5-yl)phenyl]ethyl]pyrrolidine-2-carboxamide

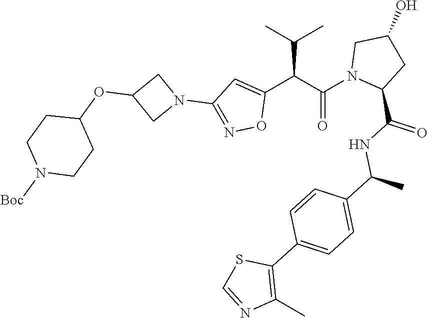 [SMILES: Cc1ncsc1-c1ccc([C@H](C)NC(=O)[C@@H]2C[C@@H](O)CN2C(=O)[C@@H](c2cc(N3CC(OC4CCN(C(=O)OC(C)(C)C)CC4)C3)no2)C(C)C)cc1]

To a solution of tert-butyl 4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxypiperidine-1-carboxylate (300 mg, 0.4 mmol) in CH₂Cl₂ (3.0 mL) was added TFA (1.5 mL, 20 mmol). The mixture was stirred at 25° C. for 30 min. The reaction mixture was concentrated in vacuum to give (2S,4R)-4-hydroxy-1-[(2R)-3-methyl-2-[3-[[3-(4-piperidyloxy) azetidin-1-yl]isoxazol-5-yl]butanoyl]-N-[(1S)-1-[4-(4-methylthiazol-5-yl)phenyl]ethyl]pyrrolidine-2-carboxamide (305 mg, crude, trifluoroacetate salt) as a yellow oil. MS (ESI) m/z: 637.2 [M+1]⁺.

Step 12: Preparation of tert-butyl 3-[7-(8-ethyl-3-hydroxy-1-naphthyl)-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate

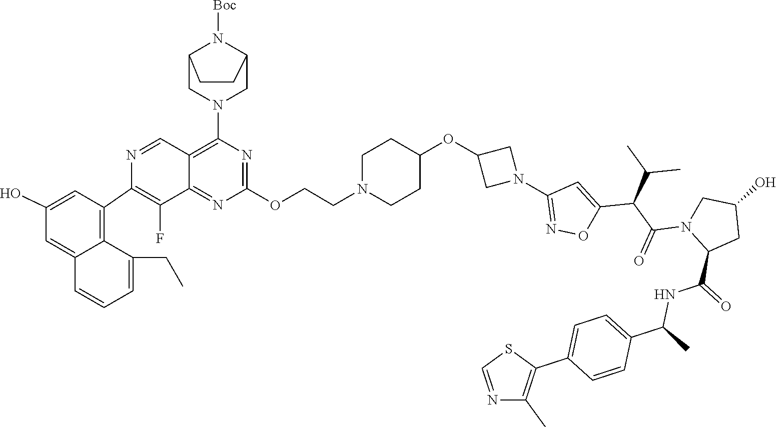 [SMILES: CCc1cccc2cc(O)cc(-c3ncc4c(N5CC6CCC(C5)N6C(=O)OC(C)(C)C)nc(OCCN5CCC(OC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (238 mg, 0.4 mmol) and (2S,4R)-4-hydroxy-1-[(2R)-3-methyl-2-[3-[[3-(4-piperidyloxy) azetidin-1-yl]isoxazol-5-yl]butanoyl]-N-[(1S)-1-[4-(4-methylthiazol-5-yl)phenyl]ethyl]pyrrolidine-2-carboxamide trifluoroacetate (304 mg, 0.4 mmol) in i-PrOH (3.0 mL) and CH₂Cl₂ (6.0 mL) was added acetic acid (116 uL, 2 mmol) and 2-methylpyridine borane (217 mg, 2 mmol). The mixture was stirred at 25° C. for 1 h, then concentrated. The residue was purified by flash column (0-6% (2.1 M NH₃ in methanol) methanol in dichloromethane) to afford tert-butyl 3-[7-(8-ethyl-3-hydroxy-1-naphthyl)-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate (300 mg, 60%) as a yellow solid. MS (ESI) m/z: 1208.5 [M+1]⁺.

Step 13: Preparation of (2S,4R)-1-[(2R)-2-[3-[[3-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]oxy]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

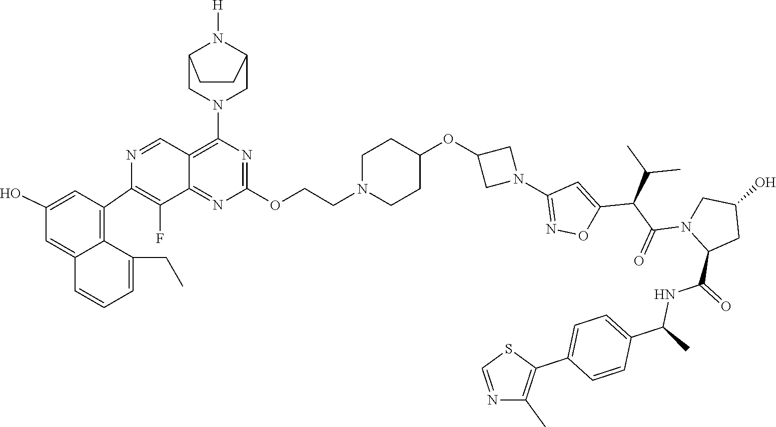 [SMILES: CCc1cccc2cc(O)cc(-c3ncc4c(N5CC6CCC(C5)N6)nc(OCCN5CCC(OC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

To a solution of tert-butyl 3-[7-(8-ethyl-3-hydroxy-1-naphthyl)-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]oxy-1-piperidyl]ethoxy]pyrido[4,3-d]pyrimidin-4-yl]-3,8-diazabicyclo[3.2.1]octane-8-carboxylate (300 mg, 0.2 mmol) in CH₂Cl₂ (3.0 mL) was added TFA (1 mL, 13 mmol). The mixture was stirred at 25° C. for 30 min, then concentrated in vacuum. The pH of the residue was adjusted to 8 with saturated aqueous NaHCO₃ solution and extracted with CH₂Cl₂/methanol (20 mL×4,V:V=10:1). The combined organic phase was washed with brine (10 mL), dried over anhydrous sodium sulfate, filtered, and concentrated in vacuum. The residue was purified by prep-HPLC (column: Xtimate C18 150*40 mm*10 um; mobile phase: [water (formic acid)-acetonitrile]; B %: 0%-36%, 36 min) to afford (2S,4R)-1-[(2R)-2-[3-[[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]oxy]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (140 mg, 48%, formic acid salt) as a white solid. MS (ESI) m/z: 1108.9 [M+1]⁺; ¹H NMR (400 MHz, CD₃OD) δ 9.07 (s, 1H), 8.87 (s, 1H), 7.63 (d, J=8.4 Hz, 1H), 7.46-7.28 (m, 6H), 7.19-7.13 (m, 1H), 7.01 (d, J=2.4 Hz, 1H), 5.88 (s, 1H), 5.06-5.00 (m, 1H), 4.75-4.66 (m, 4H), 4.60-4.48 (m, 2H), 4.43 (s, 1H), 4.15 (t, J=7.2 Hz, 2H), 3.96 (d, J=11.2 Hz, 2H), 3.89-3.73 (m, 5H), 3.66-3.48 (m, 3H), 3.14 (s, 4H), 2.76 (s, 2H), 2.47 (s, 3H), 2.39-2.15 (m, 4H), 2.04-1.90 (m, 7H), 1.73 (s, 2H), 1.51 (d, J=7.2 Hz, 3H), 1.04 (d, J=6.4 Hz, 3H), 0.93-0.84 (m, 6H).

Exemplary Synthesis of (2S,4R)-1-[(2S)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)oxy]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 216)

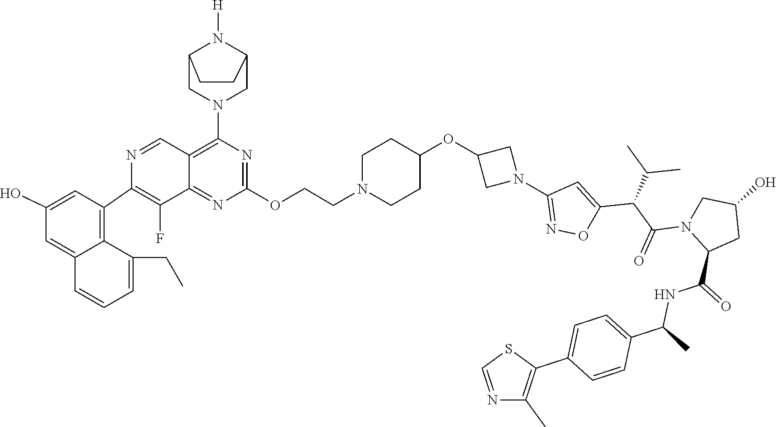 [SMILES: CCc1cccc2cc(O)cc(-c3ncc4c(N5CC6CCC(C5)N6)nc(OCCN5CCC(OC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was prepared analogously to (2S,4R)-1-[(2R)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)oxy]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide, starting from tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[[3-(4-piperidyloxy) azetidin-1-yl]isoxazol-5-yl]butanoyl]-N-[(1S)-1-[4-(4-methylthiazol-5-yl)phenyl]ethyl]pyrrolidine-2-carboxamide. (formic acid salt, white solid). MS (ESI) m/z: 1108.9 [M+1]⁺; ¹H NMR (400 MHz, CD₃OD) δ 9.07 (s, 1H), 8.86 (s, 1H), 7.63 (d, J=8.0 Hz, 1H), 7.49-7.25 (m, 6H), 7.16 (d, J=7.6 Hz, 1H), 7.01 (s, 1H), 5.89 (s, 1H), 5.04-4.98 (m, 1H), 4.75-4.66 (m, 4H), 4.60-4.53 (m, 2H), 4.42 (s, 1H), 4.18-4.07 (m, 2H), 3.96-3.94 (m, 2H), 3.87-3.63 (m, 7H), 3.5-3.54 (m, 1H), 3.14 (s, 4H), 2.77 (s, 2H), 2.46 (s, 3H), 2.40-2.19 (m, 4H), 2.04-1.87 (m, 7H), 1.73 (s, 2H), 1.5 (d, J=6.8 Hz, 3H), 1.04 (d, J=6.8 Hz, 3H), 0.93-0.83 (m, 6H).

Exemplary Synthesis of (2S,4R)-1-[(2S)-2-(3-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 217)

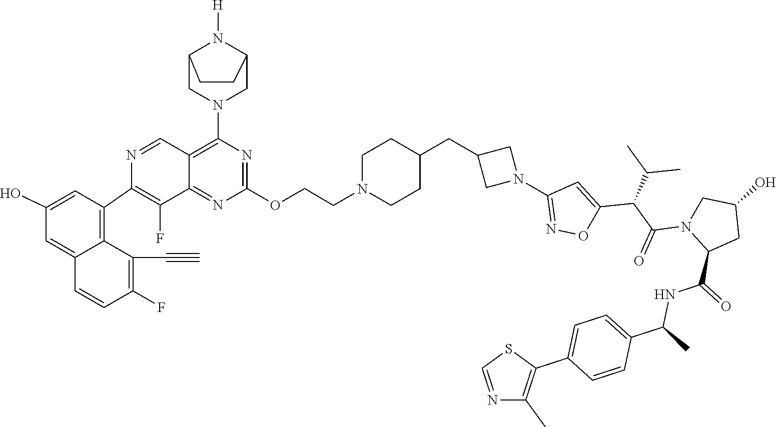 [SMILES: C#Cc1c(F)ccc2cc(O)cc(-c3ncc4c(N5CC6CCC(C5)N6)nc(OCCN5CCC(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was prepared analogously to (2S,4R)-1-((R)-2-(3-(4-((1-(2-((4-((1R,5S)-3,8-diazabicyclo[3.2.1]octan-3-yl)-7-(8-ethynyl-7-fluoro-3-hydroxynaphthalen-1-yl)-8-fluoropyrido[4,3-d]pyrimidin-2-yl)oxy)ethyl) piperidin-4-yl)methyl) piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide starting from tert-butyl 3-[8-fluoro-7-[7-fluoro-3-hydroxy-8-(2-triisopropylsilylethynyl)-1-naphthyl]-2-(2-oxoethoxy)pyrido[4,3-d]pyrimidin-4-yl]-3,8-diazabicyclo[3.2.1]octane-8-carboxylate and (2S,4R)-4-hydroxy-1-[(2S)-3-methyl-2-[3-[[3-(4-piperidylmethyl) azetidin-1-yl]isoxazol-5-yl]butanoyl]-N-[(1S)-1-[4-(4-methylthiazol-5-yl)phenyl]ethyl]pyrrolidine-2-carboxamide. (light yellow solid). MS (ESI) m/z: 1120.9 [M+H]⁺; ¹H NMR (400 MHz, CD₃OD) δ 9.00 (s, 1H), 8.87 (s, 1H), 7.87-7.83 (m, 1H), 7.47-7.31 (m, 6H), 7.21-7.20 (m, 1H), 5.86 (s, 1H), 5.01-4.96 (m, 1H), 4.65-4.55 (m, 5H), 4.43 (s, 1H), 4.02-3.97 (m, 2H), 3.73-3.65 (m, 7H), 3.54-3.49 (m, 2H), 3.37 (s, 1H), 3.08-3.06 (m, 2H), 2.86-2.83 (m, 3H), 2.47 (s, 3H), 2.39-2.31 (m, 1H), 2.25-2.12 (m, 3H), 1.99-1.93 (m, 1H), 1.88-1.78 (m, 4H), 1.66-1.55 (m, 4H), 1.49-1.47 (m, 3H), 1.29-1.25 (m, 3H), 1.05-1.03 (m, 3H), 0.91-0.89 (m, 3H).

Exemplary Synthesis of (2S,4R)-1-[(2R)-2-(3-{3-[(4-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-1-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 218)

Step 1: Preparation of 2-trimethylsilylethyl 4-(2-hydroxyethyl)piperidine-1-carboxylate

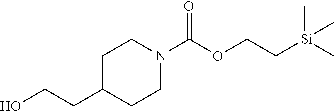 [SMILES: C[Si](C)(C)CCOC(=O)N1CCC(CCO)CC1]

To a solution of 2-(4-piperidyl) ethanol (500 mg, 4 mmol) in CH₂Cl₂ (10 mL) was added Et₃N (1.6 mL, 12 mmol) and (2,5-dioxopyrrolidin-1-yl) 2-trimethylsilylethyl carbonate (1.5 g, 5.8 mmol), the resulting suspension was stirred at 25° C. for 12 h. The reaction mixture was diluted with water (20 mL) and extracted with dichloromethane (30 mL×3). The combined organic phase was washed with brine (20 mL), dried with anhydrous Na₂SO₄, filtered, and concentrated in vacuum. The residue was purified by flash column (0-20% ethyl acetate in petroleum ether) to afford 2-trimethylsilylethyl 4-(2-hydroxyethyl)piperidine-1-carboxylate (961 mg, 91%) as a white oil. ¹H NMR (400 MHz, CHLOROFORM-d) δ 4.20-4.10 (m, 4H), 3.72 (t, J=6.4 Hz, 2H), 2.79-2.69 (m, 2H), 1.69 (br d, J=14.4 Hz, 2H), 1.57-1.52 (m, 4H), 1.20-1.08 (m, 2H), 1.06-0.93 (m, 2H), 0.04 (s, 8H).

Step 2: Preparation of tert-butyl 3-[7-[8-ethyl-3-(methoxymethoxy)-1-naphthyl]-8-fluoro-2-[2-[1-(2-trimethylsilylethoxycarbonyl)-4-piperidyl]ethoxy]pyrido[4,3-d]pyrimidin-4-yl]-3,8-diazabicyclo[3.2.1]octane-8-carboxylate

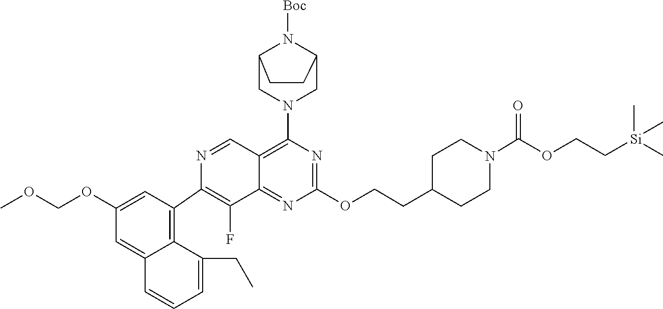

To a solution of 2-trimethylsilylethyl 4-(2-hydroxyethyl)piperidine-1-carboxylate (277 mg, 1 mmol) and tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (550 mg, 0.8 mmol) in toluene (10 mL) was added 4 A MS (1.0 g). Then the reaction mixture was cooled to 0° C., t-BuONa (203 mg, 2 mmol) was added and the resulting suspension was stirred at 0° C. for 1 h. The mixture was quenched with aq. NH₄Cl to pH 6 and diluted with water (20 mL). The aqueous phase was extracted with ethyl acetate (40 mL×3). The combined organic phase was washed with brine (30 mL), dried with anhydrous Na₂SO₄, filtered, and concentrated in vacuum. The crude product was purified by flash column (0-22% ethyl acetate in petroleum ether) to afford tert-butyl 3-[7-[8-ethyl-3-(methoxymethoxy)-1-naphthyl]-8-fluoro-2-[2-[1-(2-trimethylsilylethoxycarbonyl)-4-piperidyl]ethoxy]pyrido[4,3-d]pyrimidin-4-yl]-3,8-diazabicyclo[3.2.1]octane-8-carboxylate (445 mg, 56%) as a yellow foam. MS (ESI) m/z: 845.9 [M+H]⁺.

Step 3: Preparation of tert-butyl 3-[7-[8-ethyl-3-(methoxymethoxy)-1-naphthyl]-8-fluoro-2-[2-(4-piperidyl) ethoxy]pyrido[4,3-d]pyrimidin-4-yl]-3,8-diazabicyclo[3.2.1]octane-8-carboxylate

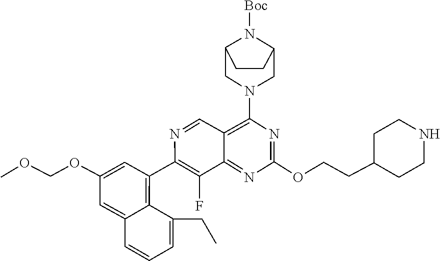 [SMILES: CCc1cccc2cc(OCOC)cc(-c3ncc4c(N5CC6CCC(C5)N6C(=O)OC(C)(C)C)nc(OCCC5CCNCC5)nc4c3F)c12]

To a solution of tert-butyl 3-[7-[8-ethyl-3-(methoxymethoxy)-1-naphthyl]-8-fluoro-2-[2-[1-(2-trimethylsilylethoxycarbonyl)-4-piperidyl]ethoxy]pyrido[4,3-d]pyrimidin-4-yl]-3,8-diazabicyclo[3.2.1]octane-8-carboxylate (445 mg, 0.5 mmol) in DMF (10 mL) was added CsF (505 mg, 3 mmol). The mixture was stirred at 90° C. for 1 h. The mixture was diluted with ethyl acetate (100 mL) and washed with water (15 mL×2). The combined organic layer was washed with brine (15 mL×2), dried over Na₂SO₄, filtered, and concentrated under reduced pressure to afford tert-butyl 3-[7-[8-ethyl-3-(methoxymethoxy)-1-naphthyl]-8-fluoro-2-[2-(4-piperidyl) ethoxy]pyrido[4,3-d]pyrimidin-4-yl]-3,8-diazabicyclo[3.2.1]octane-8-carboxylate (433.2 mg, crude) as a yellow oil, which was used in the next step without further purification. MS (ESI) m/z: 701.4 [M+H]⁺.

Step 4: Preparation of (2S,4R)-1-[(2R)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide

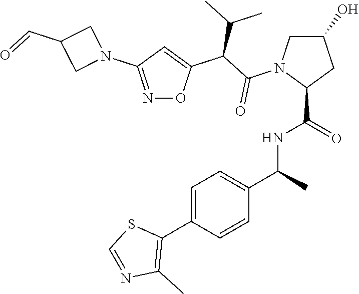 [SMILES: Cc1ncsc1-c1ccc([C@H](C)NC(=O)[C@@H]2C[C@@H](O)CN2C(=O)[C@@H](c2cc(N3CC(C=O)C3)no2)C(C)C)cc1]

To a solution of (2S,4R)-1-[(2R)-2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (220 mg, 0.4 mmol) in H₂O (0.6 mL) and acetone (3.0 mL) was added TsOH·H₂O (34 mg, 0.2 mmol). The mixture was stirred at 65° C. for 12 h, then concentrated in vacuum. The pH of the residue was adjusted to 8 by saturated aqueous NaHCO₃. The mixture was diluted with water (20 mL) and extracted with ethyl acetate (20 mL×3). The combined organic phase was washed with brine (20 mL), dried with anhydrous Na₂SO₄, filtered, and concentrated under reduced pressure to afford (2S,4R)-1-[(2R)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (206 mg, 71%) as a white solid, which was used in the next step without further purification. MS (ESI) m/z: 584.3 [M+H₂O].

Step 5: Preparation of tert-butyl 3-[7-[8-ethyl-3-(methoxymethoxy)-1-naphthyl]-8-fluoro-2-[2-[1-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-4-piperidyl]ethoxy]pyrido[4,3-d]pyrimidin-4-yl]-3,8-diazabicyclo[3.2.1]octane-8-carboxylate

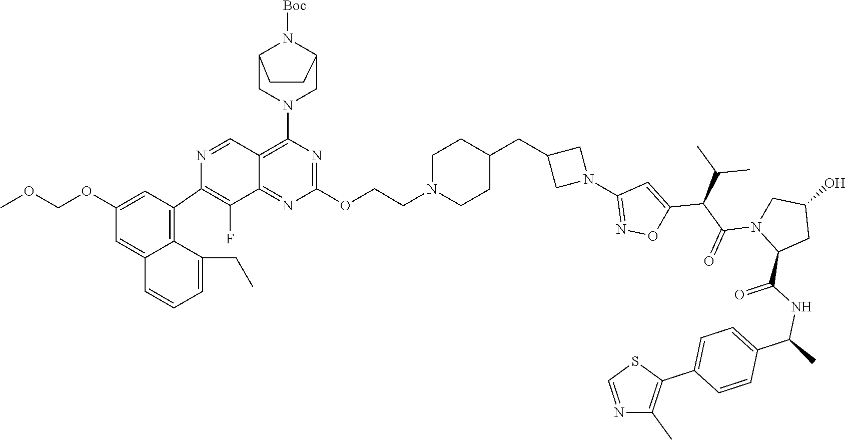 [SMILES: CCc1cccc2cc(OCOC)cc(-c3ncc4c(N5CC6CCC(C5)N6C(=O)OC(C)(C)C)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

To a solution of (2S,4R)-1-[(2R)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (206 mg, 0.2 mmol) and tert-butyl 3-[7-[8-ethyl-3-(methoxymethoxy)-1-naphthyl]-8-fluoro-2-[2-(4-piperidyl) ethoxy]pyrido[4,3-d]pyrimidin-4-yl]-3,8-diazabicyclo[3.2.1]octane-8-carboxylate (167 mg, 0.2 mmol) in CH₂Cl₂ (4.0 mL) and i-PrOH (4.0 mL) was added acetic acid (0.1 mL, 1.2 mmol) and 2-methylpyridine borane (101 mg, 0.9 mmol). The mixture was stirred at 25° C. for 1 h, then concentrated in vacuum. The residue was purified by flash column (0-8% methanol (NH₃·H₂O) in dichloromethane) to afford tert-butyl 3-[7-[8-ethyl-3-(methoxymethoxy)-1-naphthyl]-8-fluoro-2-[2-[1-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-4-piperidyl]ethoxy]pyrido[4,3-d]pyrimidin-4-yl]-3,8-diazabicyclo[3.2.1]octane-8-carboxylate (297 mg, 92%) as a yellow solid. MS (ESI) m/z: 1250.3 [M+H]⁺.

Step 6: Preparation of (2S,4R)-1-[(2R)-2-[3-[[3-[4-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-1-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

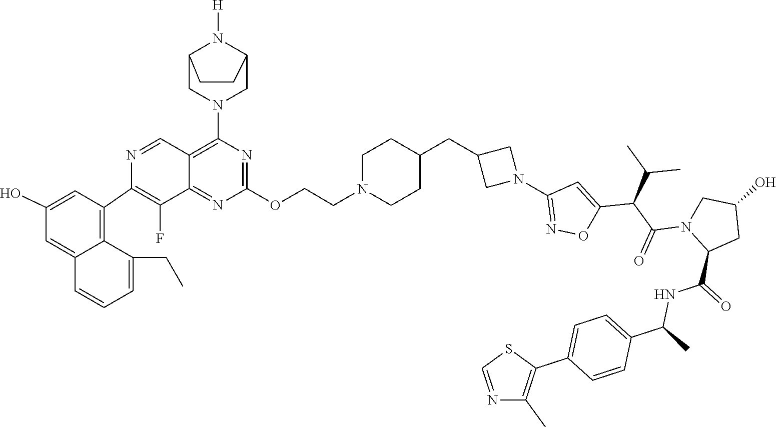 [SMILES: CCc1cccc2cc(O)cc(-c3ncc4c(N5CC6CCC(C5)N6)nc(OCCN5CCC(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

To a solution of tert-butyl 3-[7-[8-ethyl-3-(methoxymethoxy)-1-naphthyl]-8-fluoro-2-[2-[1-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]-4-piperidyl]ethoxy]pyrido[4,3-d]pyrimidin-4-yl]-3,8-diazabicyclo[3.2.1]octane-8-carboxylate (297 mg, 0.2 mmol) in CH₂Cl₂ (3.0 mL) was added TFA (1.5 mL, 20 mmol). The mixture was stirred at 25° C. for 1 h, then concentrated under reduced pressure. The pH of the residue was adjusted to 8 by saturated aqueous NaHCO₃. The resulting mixture was extracted with dichloromethane/methanol (110 mL, 10:1, V/V). The combined organic phase was washed with brine (30 mL), dried with anhydrous Na₂SO₄, filtered, and concentrated in vacuum. The crude product was purified by prep-HPLC (Column: Phenomenex luna C18 150*40 mm*10 um; Eluent: gradient 34%-74% acetonitrile in water (NH₄HCO₃); Gradient time: 36 min; Hold time: 3 min; Flow rate: 25 mL/min) to afford (2S,4R)-1-[(2R)-2-[3-[[3-[[4-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-1-piperidyl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (60.1 mg, 25%) as a white solid. MS (ESI) m/z: 1106.5 [M+H]⁺; ¹H NMR (400 MHz, METHANOL-d4) δ 9.03 (s, 1H), 8.88 (s, 1H), 7.63 (d, J=8.0 Hz, 1H), 7.46-7.41 (m, 3H), 7.41-7.32 (m, 2H), 7.29 (d, J=2.4 Hz, 1H), 7.16 (d, J=7.2 Hz, 1H), 7.01 (d, J=2.8 Hz, 1H), 5.87-5.80 (m, 1H), 5.03 (q, J=6.8 Hz, 1H), 4.59-4.46 (m, 4H), 4.43 (br s, 1H), 4.10-4.04 (m, 2H), 3.83 (dd, J=4.0, 10.8 Hz, 1H), 3.76-3.57 (m, 8H), 3.11-3.01 (m, 1H), 2.93 (br d, J=10.8 Hz, 2H), 2.76-2.69 (m, 2H), 2.48 (s, 3H), 2.39-2.25 (m, 3H), 2.22-2.08 (m, 3H), 2.00-1.92 (m, 1H), 1.91-1.76 (m, 8H), 1.59-1.50 (m, 3H), 1.41-1.27 (m, 3H), 1.04 (d, J=6.4 Hz, 3H), 0.92-0.85 (m, 6H).

Exemplary Synthesis of (2S,4R)-1-[(2S)-2-(3-{3-[(4-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-1-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 219)

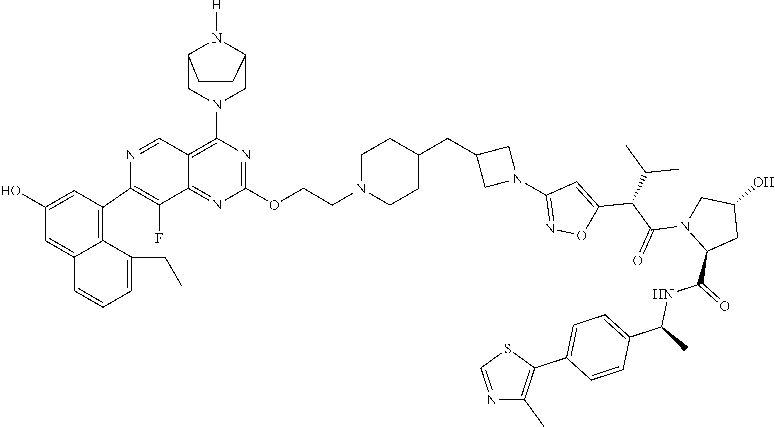 [SMILES: CCc1cccc2cc(O)cc(-c3ncc4c(N5CC6CCC(C5)N6)nc(OCCN5CCC(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was prepared in an analogous manner to (2S,4R)-1-[(2R)-2-(3-{3-[(4-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-1-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide, starting from (2S,4R)-1-[(2S)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-[8-ethyl-3-(methoxymethoxy)-1-naphthyl]-8-fluoro-2-[2-(4-piperidyl) ethoxy]pyrido[4,3-d]pyrimidin-4-yl]-3,8-diazabicyclo[3.2.1]octane-8-carboxylate. (white solid). MS (ESI) m/z: 1106.9 [M+H]⁺; ¹H NMR (400 MHz, MeOD-d₄) δ 9.03 (s, 1H), 8.89-8.86 (m, 1H), 7.62 (d, J=8.4 Hz, 1H), 7.48-7.40 (m, 2H), 7.39-7.32 (m, 3H), 7.28 (d, J=2.4 Hz, 1H), 7.16 (d, J=7.6 Hz, 1H), 7.01 (d, J=2.8 Hz, 1H), 5.87 (s, 1H), 4.99 (d, J=6.8 Hz, 1H), 4.57-4.51 (m, 4H), 4.43 (br s, 1H), 4.09-4.01 (m, 2H), 3.74-3.58 (m, 9H), 3.06-2.97 (m, 1H), 2.90 (br d, J=10.4 Hz, 2H), 2.72-2.64 (m, 2H), 2.49-2.46 (m, 3H), 2.38-2.18 (m, 4H), 2.13-2.03 (m, 2H), 1.96 (ddd, J=4.8, 8.4, 13.2 Hz, 1H), 1.90-1.82 (m, 3H), 1.82-1.75 (m, 5H), 1.49 (d, J=7.2 Hz, 3H), 1.41-1.27 (m, 4H), 1.05 (d, J=6.6 Hz, 3H), 0.92-0.87 (m, 6H).

Exemplary Synthesis of (2S,4R)-1-[(2R)-2-(3-{3-[(4-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperazin-1-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 220)

Step 1: Preparation of tert-butyl 3-[7-[8-ethyl-3-(methoxymethoxy)-1-naphthyl]-8-fluoro-2-[2-[[4-(2-trimethylsilylethoxycarbonyl) piperazin-1-yl]ethoxy]pyrido[4,3-d]pyrimidin-4-yl]-3,8-diazabicyclo[3.2.1]octane-8-carboxylate

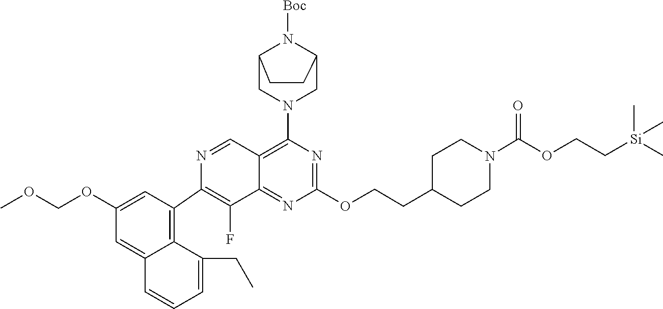

To a solution of 2-trimethylsilylethyl 4-(2-hydroxyethyl) piperazine-1-carboxylate (455 mg, 92% purity, 1.7 mmol), 4 A molecular sieves (1 g) and tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (900 mg, 1.4 mmol) in toluene (16 mL) was added t-BuONa (332 mg, 3.5 mmol) at 0° C. under N₂. The mixture was stirred at 0° C. for 40 min. The mixture was quenched with aq. NH₄Cl to pH 6 and diluted with water (20 mL). The aqueous phase was extracted with ethyl acetate (40 mL×3). The combined organic phase was washed with brine (30 mL), dried with anhydrous Na₂SO₄, filtered and concentrated. The residue was purified by column chromatography (0-50% ethyl acetate in petroleum ether) to afford tert-butyl 3-[7-[8-ethyl-3-(methoxymethoxy)-1-naphthyl]-8-fluoro-2-[2-[[4-(2-trimethylsilylethoxycarbonyl) piperazin-1-yl]ethoxy]pyrido[4,3-d]pyrimidin-4-yl]-3,8-diazabicyclo[3.2.1]octane-8-carboxylate (972 mg, 72%) as a yellow solid. MS (ESI) m/z: 846.4 [M+1]⁺.

Step 2: Preparation of tert-butyl 3-[7-[8-ethyl-3-(methoxymethoxy)-1-naphthyl]-8-fluoro-2-(2-piperazin-1-ylethoxy)pyrido[4,3-d]pyrimidin-4-yl]-3,8-diazabicyclo[3.2.1]octane-8-carboxylate

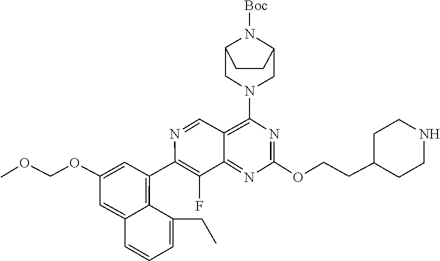 [SMILES: CCc1cccc2cc(OCOC)cc(-c3ncc4c(N5CC6CCC(C5)N6C(=O)OC(C)(C)C)nc(OCCC5CCNCC5)nc4c3F)c12]

A mixture of tert-butyl 3-[7-[8-ethyl-3-(methoxymethoxy)-1-naphthyl]-8-fluoro-2-[2-[[4-(2-trimethylsilylethoxycarbonyl) piperazin-1-yl]ethoxy]pyrido[4,3-d]pyrimidin-4-yl]-3,8-diazabicyclo[3.2.1]octane-8-carboxylate (300 mg, 0.4 mmol) and CsF (377 mg, 2.5 mmol) in DMF (3 mL) was stirred at 90° C. for 1 h. The reaction mixture was diluted with water (20 mL) and extracted with ethyl acetate (30 mL×3). The combined extracts were washed with water (10 mL×5) and brine (15 mL×2), dried over anhydrous Na₂SO₄, filtered, and concentrated to afford tert-butyl3-[7-[8-ethyl-3-(methoxymethoxy)-1-naphthyl]-8-fluoro-2-(2-piperazin-1-ylethoxy)pyrido[4,3-d]pyrimidin-4-yl]-3,8-diazabicyclo[3.2.1]octane-8-carboxylate (312 mg, crude) as a yellow gum, which was used directly in the next step. MS (ESI) m/z: 702.1 [M+1]⁺.

Step 3: Preparation of benzyl 3-(dimethoxymethyl)azetidine-1-carboxylate

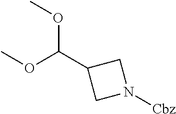 [SMILES: COC(OC)C1CN(C(=O)OCc2ccccc2)C1]

To a mixture of benzyl 3-formylazetidine-1-carboxylate (4.90 g, 22 mmol) in MeOH (80 mL) was added TsOH·H₂O (213 mg, 1 mmol) and trimethoxymethane (11.86 g, 112 mmol). The mixture was stirred at 25° C. for 12 h, then concentrated in vacuum. The residue was diluted with water (40 mL) and extracted with ethyl acetate (50 mL×3). The combined organic phase was washed with brine (30 mL), dried with anhydrous Na₂SO₄, filtered, and concentrated under reduced pressure. The residue was purified by column chromatography (0-28% then up to 35% ethyl acetate in petroleum ether) to afford benzyl 3-(dimethoxymethyl)azetidine-1-carboxylate (5.13 g, 87%) as a colorless oil. MS (ESI) m/z: 266.1 [M+1]⁺; 1H NMR (400 MHz, CDCl₃) δ 7.42-7.28 (m, 5H), 5.10 (s, 2H), 4.53 (d, J=7.2 Hz, 1H), 4.09-4.01 (m, 2H), 3.87 (dd, J=5.6, 8.8 Hz, 2H), 3.36 (s, 6H), 2.92-2.79 (m, 1H).

Step 4: Preparation of 3-(dimethoxymethyl)azetidine

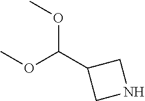

To a solution of benzyl 3-(dimethoxymethyl)azetidine-1-carboxylate (5.13 g, 19 mmol) in 2,2,2-trifluoroethanol (90 mL) was added Pd/C (1 g, 10%). The mixture was degassed under vacuum and purged with H₂ several times. The resulting suspension was stirred under H₂ (15 psi) at 45° C. for 16 h. The mixture was filtered through Celite pad and washed with ethyl acetate (100 mL). The filtrate was concentrated to give 3-(dimethoxymethyl)azetidine (2.47 g, crude) as a yellow oil, which was used directly in the next step. ¹H NMR (400 MHz, CDCl₃) δ 4.59 (d, J=6.8 Hz, 1H), 3.75-3.69 (m, 2H), 3.66-3.59 (m, 2H), 3.35 (s, 6H), 3.08-2.97 (m, 1H).

Step 5: Preparation of methyl 2-[3-[[3-(dimethoxymethyl) azetidin-1-yl]isoxazol-5-yl]-3-methyl-butanoate

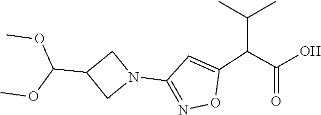 [SMILES: COC(OC)C1CN(c2cc(C(C(=O)O)C(C)C)on2)C1]

To a mixture of 3-(dimethoxymethyl)azetidine (2.47 g, 19 mmol) in DMA (90 mL) were added 4 A molecular sieves (10 g) and triethylamine (7.86 mL, 56 mmol) at 130° C. under N₂, the mixture was stirred at this temperature for 5 min, then methyl 3-methyl-2-[3-(1,1,2,2,3,3,4,4,4-nonafluorobutylsulfonyloxy)isoxazol-5-yl]butanoate (9.06 g, 19 mmol) in DMA (10 mL) was added dropwise at 130° C. in 3 h. The mixture was stirred at 130° C. for 12 h. the reaction was diluted with ethyl acetate (30 mL), filtered through celite pad under vacuum and washed with ethyl acetate (50 mL). The filtrate was diluted with water (50 mL) and extracted with ethyl acetate (80 mL×4). The combined organic layers were washed with water (25 mL×8) and brine (30 mL×2), dried with anhydrous Na₂SO₄, filtered and concentrated. The residue was purified column chromatography (0-24% ethyl acetate in petroleum ether) to afford methyl 2-[3-[[3-(dimethoxymethyl) azetidin-1-yl]isoxazol-5-yl]-3-methyl-butanoate (1.67 g, 28%) as a yellow oil. MS (ESI) m/z: 313.1 [M+1]⁺.

Step 6: Preparation of (2S,4R)-4-hydroxy-1-[(2S)-3-methyl-2-[3-[2-(4-piperidyl) ethoxy]isoxazol-5-yl]butanoyl]-N-[(1S)-1-[4-(4-methylthiazol-5-yl)phenyl]ethyl]pyrrolidine-2-carboxamide

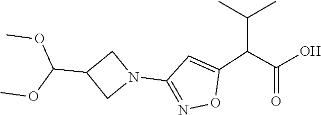 [SMILES: COC(OC)C1CN(c2cc(C(C(=O)O)C(C)C)on2)C1]

To a mixture of methyl 2-[3-[[3-(dimethoxymethyl) azetidin-1-yl]isoxazol-5-yl]-3-methyl-butanoate (1.57 g, 5 mmol) in THF (16 mL) and H₂O (8 mL) was added LiOH·H₂O (738 mg, 18 mmol) at 25° C. The mixture was stirred at 25° C. for 12 h, then concentrated in vacuum. The pH of the residue was adjusted to 3 with 2 M hydrochloric acid. The resulting mixture was extracted with dichloromethane (30 mL×3), the combined organic phase was washed with brine (20 mL), dried with anhydrous Na₂SO₄, filtered and concentrated to afford 2-[3-[[3-(dimethoxymethyl) azetidin-1-yl]isoxazol-5-yl]-3-methyl-butanoic acid (1.5 g, crude) as a yellow, which was used directly in the next step. MS (ESI) m/z: 298.9 [M+1]⁺.

Step 7: Preparation of (2S,4R)-1-[2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

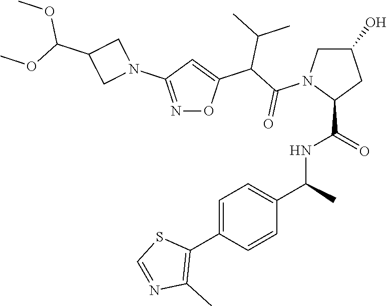 [SMILES: COC(OC)C1CN(c2cc(C(C(=O)N3C[C@H](O)C[C@H]3C(=O)N[C@@H](C)c3ccc(-c4scnc4C)cc3)C(C)C)on2)C1]

To a solution of 2-[3-[[3-(dimethoxymethyl) azetidin-1-yl]isoxazol-5-yl]-3-methyl-butanoic acid (1.5 g, 5 mmol) and (2S,4R)-4-hydroxy-N-[(1S)-1-[4-(4-methylthiazol-5-yl)phenyl]ethyl]pyrrolidine-2-carboxamide hydrochloride (2.71 g, 5 mmol) in CH₂Cl₂ (30 mL) was added DIEA (4.38 mL, 25 mmol) and HATU (2.10 g, 5 mmol). The mixture was stirred at 25° C. for 12 h. The reaction mixture was diluted with water (30 mL) and extracted with dichloromethane (40 mL×3). The combined extracts were washed with brine (30 mL), dried over anhydrous Na₂SO₄, filtered and concentrated. The residue was purified by column chromatography (0-100% ethyl acetate in petroleum ether) to afford (2S,4R)-1-[2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (2.66 g, 86%) as a yellow solid. MS (ESI) m/z: 612.1 [M+1]⁺.

Step 8: Preparation of (2S,4R)-1-[(2S)-2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide and (2S,4R)-1-[(2R)-2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

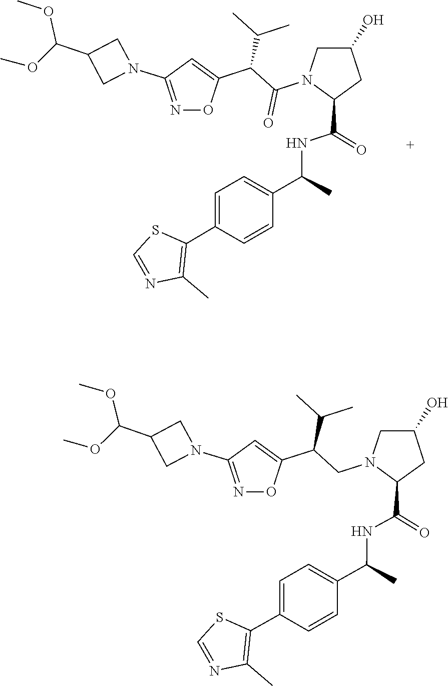 [SMILES: COC(OC)C1CN(c2cc([C@@H](C(=O)N3C[C@H](O)C[C@H]3C(=O)N[C@@H](C)c3ccc(-c4scnc4C)cc3)C(C)C)on2)C1 ; COC(OC)C1CN(c2cc([C@H](CN3C[C@H](O)C[C@H]3C(=O)N[C@@H](C)c3ccc(-c4scnc4C)cc3)C(C)C)on2)C1]

(2S,4R)-1-[2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (2.4 g) was separated by chiral SFC (column: DAICEL CHIRALPAK AD (250 mm*50 mm, 10 um); mobile phase: [0.1% NH₃H₂O-IPA]; B %: 50%-50%). Fraction 1 was obtained as (2S,4R)-1-[(2S)-2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (1.3 g, 54%) as a white solid. Fraction 2 was obtained as (2S,4R)-1-[(2R)-2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (880 mg, 37%) as a white solid. MS (ESI) m/z: 612.3 [M+1]⁺.

Step 9: Preparation of (2S,4R)-1-[(2R)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide

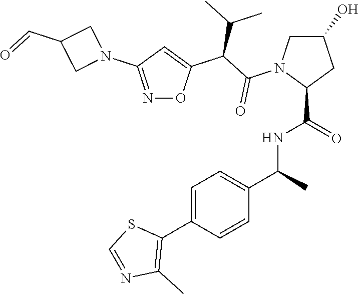 [SMILES: Cc1ncsc1-c1ccc([C@H](C)NC(=O)[C@@H]2C[C@@H](O)CN2C(=O)[C@@H](c2cc(N3CC(C=O)C3)no2)C(C)C)cc1]

To a solution of (2S,4R)-1-[(2R)-2-[3-[[3-(dimethoxymethyl) azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (220 mg, 0.4 mmol) in H₂O (0.8 mL) and acetone (4 mL) was added TsOH·H₂O (34 mg, 0.2 mmol). The mixture was stirred at 65° C. for 6 h, then concentrated in vacuum. The pH of the residue was adjusted to 8 by saturated aqueous NaHCO₃ solution. The mixture was diluted with water (10 mL) and extracted with ethyl acetate (30 mL×3). The combined organic phase was washed with brine (20 mL), dried with anhydrous Na₂SO₄, filtered and concentrated to afford (2S,4R)-1-[(2R)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (210 mg, crude) as a white solid, which was used directly in the next step. MS (ESI) m/z: 584.0 [M+18]⁺.

Step 10: Preparation of tert-butyl 3-[7-[8-ethyl-3-(methoxymethoxy)-1-naphthyl]-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperazin-1-yl]ethoxy]pyrido[4,3-d]pyrimidin-4-yl]-3,8-diazabicyclo[3.2.1]octane-8-carboxylate

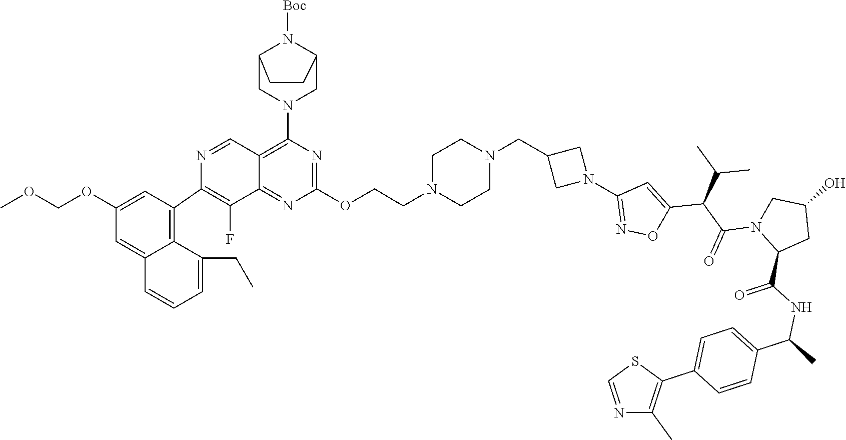 [SMILES: CCc1cccc2cc(OCOC)cc(-c3ncc4c(N5CC6CCC(C5)N6C(=O)OC(C)(C)C)nc(OCCN5CCN(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

To a mixture of (2S,4R)-1-[(2R)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide (210 mg, 0.4 mmol) and tert-butyl 3-[7-[8-ethyl-3-(methoxymethoxy)-1-naphthyl]-8-fluoro-2-(2-piperazin-1-ylethoxy)pyrido[4,3-d]pyrimidin-4-yl]-3,8-diazabicyclo[3.2.1]octane-8-carboxylate (300 mg, 0.4 mmol) in CH₂Cl₂ (4 mL) and i-PrOH (4 mL) were added AcOH (106 uL, 1.9 mmol) and 2-methylpyridine borane (159 mg, 1.5 mmol) at 25° C. The mixture was stirred at 25° C. for 1 h, then concentrated in vacuum. The pH of the residue was adjusted 10 by trimethylamine and purified by column chromatography (0-3% then up to 5% methanol (1N NH₃ as additive) in dichloromethane) to afford tert-butyl 3-[7-[8-ethyl-3-(methoxymethoxy)-1-naphthyl]-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperazin-1-yl]ethoxy]pyrido[4,3-d]pyrimidin-4-yl]-3,8-diazabicyclo[3.2.1]octane-8-carboxylate (372 mg, 80%) as a yellow solid. MS (ESI) m/z: 626.4 [M/2+1]⁺.

Step 11: Preparation of (2S,4R)-1-[(2R)-2-[3-[[3-[4-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]piperazin-1-yl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide

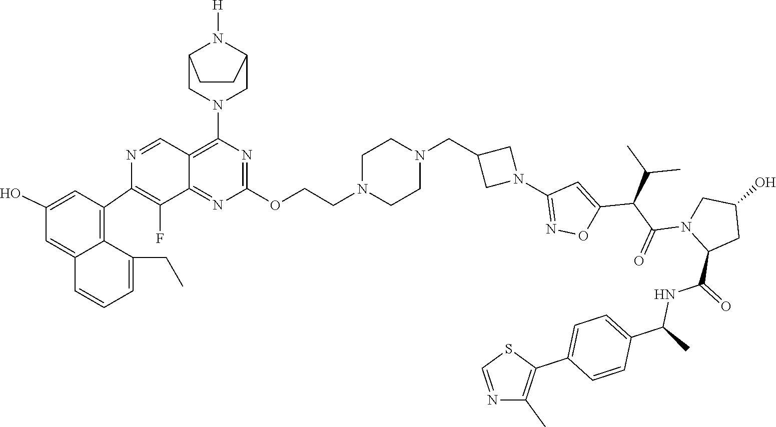 [SMILES: CCc1cccc2cc(O)cc(-c3ncc4c(N5CC6CCC(C5)N6)nc(OCCN5CCN(CC6CN(c7cc([C@H](C(=O)N8C[C@H](O)C[C@H]8C(=O)N[C@@H](C)c8ccc(-c9scnc9C)cc8)C(C)C)on7)C6)CC5)nc4c3F)c12]

To a mixture of tert-butyl 3-[7-[8-ethyl-3-(methoxymethoxy)-1-naphthyl]-8-fluoro-2-[2-[[4-[[1-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]azetidin-3-yl]methyl]piperazin-1-yl]ethoxy]pyrido[4,3-d]pyrimidin-4-yl]-3,8-diazabicyclo[3.2.1]octane-8-carboxylate (372 mg, 0.3 mmol) in CH₂Cl₂ (3 mL) was added TFA (1 mL). The mixture was stirred at 25° C. for 2 h. The pH of the mixture was adjusted to 8 by saturated aqueous NaHCO₃ solution. The resulting mixture was extracted with dichloromethane/methanol (100 mL, 10:1, V/V). The combined organic phase was washed with brine (30 mL), dried with anhydrous Na₂SO₄, filtered and concentrated. The residue was purified by preparative HPLC (column: Xtimate C18 150*40 mm*10 um; mobile phase: [water (formic acid)-acetonitrile]; B %: 0%-36%, 36 min), the crude product was further purified by SFC (column: ChiralPak IH, 250*30 mm, 10 um; mobile phase: [acetonitrile/ethanol (0.1% NH₃H₂O)]; B %: 25%-25%, 8 min). Pure fractions were combined and concentrated under reduced pressure, the residue was washed with n-hexane (8 mL×3), dissolved in water and acetonitrile, lyophilized to afford (2S,4R)-1-[(2R)-2-[3-[[3-[[4-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]piperazin-1-yl]methyl]azetidin-1-yl]isoxazol-5-yl]-3-methyl-butanoyl]-4-hydroxy-N-[(1S)-1-[4-(4-methylthiazol-5-yl)phenyl]ethyl]pyrrolidine-2-carboxamide (103.7 mg, 0.31 formic acid salt, 31%) as a white solid. MS (ESI) m/z: 1107.9 [M+1]⁺; 1H NMR (400 MHz, CD₃OD) δ 9.05 (s, 1H), 8.87 (s, 1H), 7.63 (d, J=8.2 Hz, 1H), 7.46-7.33 (m, 5H), 7.29 (d, J=2.4 Hz, 1H), 7.16 (d, J=7.2 Hz, 1H), 7.01 (d, J=2.4 Hz, 1H), 5.88-5.77 (m, 1H), 5.07-4.99 (m, 1H), 4.69-4.55 (m, 7H), 4.50 (t, J=8.0 Hz, 1H), 4.46-4.38 (m, 1H), 4.05 (t, J=7.6 Hz, 2H), 3.85-3.72 (m, 5H), 3.67-3.56 (m, 4H), 3.03 (td, J=6.8, 14.0 Hz, 1H), 2.90 (t, J=5.2 Hz, 2H), 2.70 (d, J=7.2 Hz, 4H), 2.59-2.53 (m, 2H), 2.48 (s, 3H), 2.42-2.13 (m, 5H), 1.99-1.86 (m, 5H), 1.60-1.50 (m, 3H), 1.04 (d, J=6.4 Hz, 3H), 0.92-0.86 (m, 6H).

Exemplary Synthesis of (2S,4R)-1-[(2S)-2-(3-{3-[(4-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperazin-1-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Compound 221)

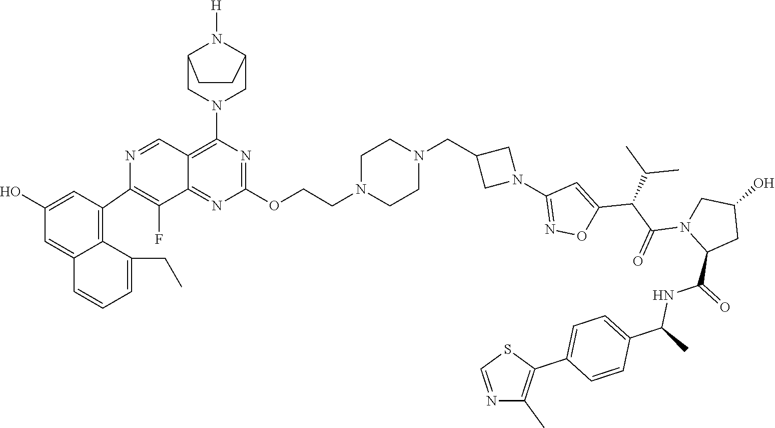 [SMILES: CCc1cccc2cc(O)cc(-c3ncc4c(N5CC6CCC(C5)N6)nc(OCCN5CCN(CC6CN(c7cc([C@@H](C(=O)N8C[C@H](O)C[C@H]8C(=O)N[C@@H](C)c8ccc(-c9scnc9C)cc8)C(C)C)on7)C6)CC5)nc4c3F)c12]

The title compound was prepared analogously to (2S,4R)-1-[(2R)-2-(3-{3-[(4-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperazin-1-yl)methyl]azetidin-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide, starting from 2S,4R)-1-[(2S)-2-[3-(3-formylazetidin-1-yl)isoxazol-5-yl]-3-methyl-butanoyl]-4-hydroxy-N-[(1S)-1-[4-(4-methylthiazol-5-yl)phenyl]ethyl]pyrrolidine-2-carboxamide and tert-butyl 3-[7-[8-ethyl-3-(methoxymethoxy)-1-naphthyl]-8-fluoro-2-(2-piperazin-1-ylethoxy)pyrido[4,3-d]pyrimidin-4-yl]-3,8-diazabicyclo[3.2.1]octane-8-carboxylate. (formic acid salt, white solid). MS (ESI) m/z: 1108.0 [M+1]⁺; 1H NMR (400 MHz, CD₃OD) δ 9.05 (s, 1H), 8.91-8.83 (m, 1H), 8.51 (s, 0.36H), 7.63 (d, J=7.6 Hz, 1H), 7.48-7.40 (m, 2H), 7.39-7.32 (m, 3H), 7.29 (d, J=2.4 Hz, 1H), 7.16 (d, J=7.0 Hz, 1H), 7.01 (d, J=2.4 Hz, 1H), 5.90-5.81 (m, 1H), 4.99 (q, J=6.8 Hz, 1H), 4.68-4.55 (m, 8H), 4.43 (s, 1H), 4.08-3.98 (m, 2H), 3.84-3.59 (m, 9H), 3.03-2.95 (m, 1H), 2.92-2.86 (m, 2H), 2.74-2.64 (m, 4H), 2.54 (d, J=3.6 Hz, 2H), 2.49-2.45 (m, 3H), 2.41-2.17 (m, 5H), 2.00-1.85 (m, 5H), 1.59-1.47 (m, 3H), 1.05 (d, J=6.4 Hz, 3H), 0.92-0.82 (m, 6H).

Exemplary Synthesis of [(2S,5S)-5-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-1-methylpyrrolidin-2-yl]methyl (3R)-4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}-3-methylpiperazine-1-carboxylate (Compound 222)

Step 1: Preparation of tert-butyl 3-[7-(8-ethyl-1-naphthyl)-8-fluoro-2-[[(2S,5S)-5-[[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate

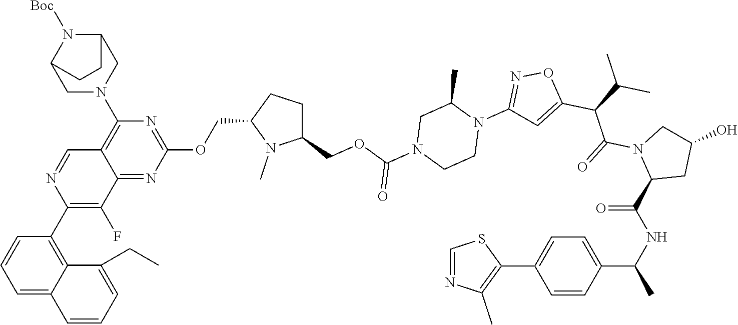 [SMILES: CCc1cccc2cccc(-c3ncc4c(N5CC6CCC(C5)N6C(=O)OC(C)(C)C)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)[C@H](C)C6)N5C)nc4c3F)c12]

To a mixture of tert-butyl 3-[7-(8-ethyl-1-naphthyl)-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (200 mg, 285 umol) in THF (10 mL) was added TEA (200 uL, 1.4 mmol), DMAP (6.9 mg, 57 umol) and (4-nitrophenyl) carbonochloridate (109 mg, 541 umol), the resulting suspension was stirred at 25° C. for 15 h. Then (2S,4R)-4-hydroxy-1-[(2R)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide (248 mg, 427 umol) was added, the resulting suspension was stirred at 25° C. for 2 h. The reaction mixture was filtered and concentrated under reduced pressure. The residue was purified by column chromatography (0-6% MeOH in CH₂Cl₂) to afford tert-butyl 3-[7-(8-ethyl-1-naphthyl)-8-fluoro-2-[[(2S,5S)-5-[[(3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate (388 mg, 87%) as a yellow solid. MS (ESI) m/z: 1263.3 [M+H]⁺.

Step 2: Preparation of [(2S,5S)-5-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1-methyl-pyrrolidin-2-yl]methyl (3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carboxylate

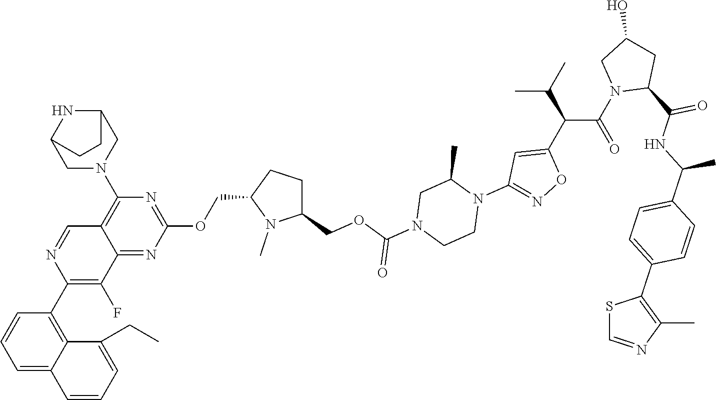 [SMILES: CCc1cccc2cccc(-c3ncc4c(N5CC6CCC(C5)N6)nc(OC[C@@H]5CC[C@@H](COC(=O)N6CCN(c7cc([C@H](C(=O)N8C[C@H](O)C[C@H]8C(=O)N[C@@H](C)c8ccc(-c9scnc9C)cc8)C(C)C)on7)[C@H](C)C6)N5C)nc4c3F)c12]

To a reaction mixture of tert-butyl 3-[7-(8-ethyl-1-naphthyl)-8-fluoro-2-[[(2S,5S)-5-[[(3R)-4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate (388 mg, 249 umol) in CH₂Cl₂ (4 mL) was added HCl in dioxane (4 M, 2 mL), the resulting suspension was stirred at 25° C. for 0.25 h. The reaction mixture was diluted with petroleum ether (30 mL), the precipitate was collected, dissolved with THF (50 mL), adjusted the pH to 8 with triethylamine. The mixture was filtered and concentrated under reduced pressure. The crude product was purified by prep-HPLC (column: Xtimate C18 150*40 mm*10 um; mobile phase: [water (formic acid)-acetonitrile]; Begin B: 0; End B: 38; Gradient Time (min): 36; 100% B Hold Time (min): 3; Flow Rate (ml/min): 60) to afford [(2S,5S)-5-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1-methyl-pyrrolidin-2-yl]methyl (3R)-4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]-3-methyl-piperazine-1-carboxylate (160.3 mg, 55%) as a white solid. MS (ESI) m/z: 1164.0 [M+H]⁺; 1H NMR (400 MHz, DMSO-d₆) δ 9.10 (s, 1H), 8.98 (s, 1H), 8.41 (d, J=7.6 Hz, 1H), 8.07 (d, J=8.0 Hz, 1H), 7.91 (d, J=8.0 Hz, 1H), 7.58 (t, J=7.6 Hz, 1H), 7.51 (t, J=7.6 Hz, 1H), 7.46-7.41 (m, 3H), 7.40-7.32 (m, 3H), 6.16-5.96 (m, 1H), 4.91 (quin, J=7.2 Hz, 1H), 4.51-4.34 (m, 4H), 4.31-4.21 (m, 2H), 4.13-3.63 (m, 10H), 3.41-2.90 (m, 10H), 2.49-2.43 (m, 6H), 2.40-2.18 (m, 3H), 2.06-1.91 (m, 3H), 1.78 (ddd, J=4.8, 8.0, 12.8 Hz, 1H), 1.73-1.55 (m, 6H), 1.47-1.33 (m, 3H), 1.03-0.90 (m, 6H), 0.89-0.74 (m, 6H).

Exemplary Synthesis of [(2S,5S)-5-{[(4-{3,8-diazabicyclo[3.2.1]octan-3-yl}-7-(8-ethylnaphthalen-1-yl)-8-fluoropyrido[4,3-d]pyrimidin-2-yl)oxy]methyl}-1-methylpyrrolidin-2-yl]methyl 3-({5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}oxy)azetidine-1-carboxylate (Compound 223)

Step 1: Preparation of tert-butyl 3-[2-[[(2S,5S)-5-[[tert-butyl(diphenyl)silyl]oxymethyl]-1-methyl-pyrrolidin-2-yl]methoxy]-7-(8-ethyl-1-naphthyl)-8-fluoro-pyrido[4,3-d]pyrimidin-4-yl]-3,8-diazabicyclo[3.2.1]octane-8-carboxylate

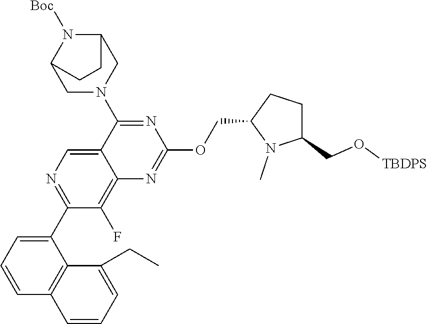

To a reaction mixture of tert-butyl 3-[7-(8-ethyl-1-naphthyl)-8-fluoro-2-methylsulfonyl-pyrido[4,3-d]pyrimidin-4-yl]-3,8-diazabicyclo[3.2.1]octane-8-carboxylate (800 mg, 1.3 mmol) and [(2S,5S)-5-[[tert-butyl(diphenyl)silyl]oxymethyl]-1-methyl-pyrrolidin-2-yl]methanol (567 mg, 1.5 mmol) in dioxane (16 mL) were added 4 A MS (2 g). Then reaction mixture was cooled to 0° C., t-BuONa (370 mg, 3.9 mmol) was added. The resulting suspension was stirred at 0° C. for 0.5 h. The reaction was quenched with HCl (2 M) to pH 6. Diluted with ethyl acetate (100 mL), washed with water (50 mL×2), brine (50 mL×2), dried over Na₂SO₄, filtered and concentrated under reduced pressure to afford tert-butyl 3-[2-[[(2S,5S)-5-[[tert-butyl(diphenyl)silyl]oxymethyl]-1-methyl-pyrrolidin-2-yl]methoxy]-7-(8-ethyl-1-naphthyl)-8-fluoro-pyrido[4,3-d]pyrimidin-4-yl]-3,8-diazabicyclo[3.2.1]octane-8-carboxylate (1.15 g, crude) as a yellow solid, which was used in the next step directly. MS (ESI) m/z: 895.5 [M+H]⁺.

Step 2: Preparation of tert-butyl 3-[7-(8-ethyl-1-naphthyl)-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate

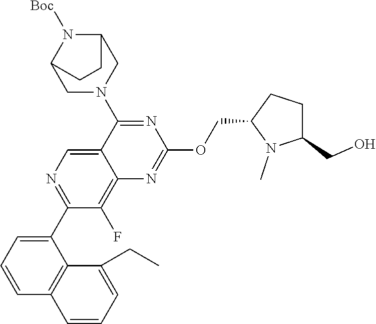 [SMILES: CCc1cccc2cccc(-c3ncc4c(N5CC6CCC(C5)N6C(=O)OC(C)(C)C)nc(OC[C@@H]5CC[C@@H](CO)N5C)nc4c3F)c12]

To a reaction mixture of tert-butyl 3-[2-[[(2S,5S)-5-[[tert-butyl(diphenyl)silyl]oxymethyl]-1-methyl-pyrrolidin-2-yl]methoxy]-7-(8-ethyl-1-naphthyl)-8-fluoro-pyrido[4,3-d]pyrimidin-4-yl]-3,8-diazabicyclo[3.2.1]octane-8-carboxylate (1.15 g, 1.3 mmol) in DMF (20 mL) was added CsF (976 mg, 6.4 mmol), the resulting suspension was stirred at 25° C. for 15 h. The reaction mixture was diluted with water (20 mL) and EtOAc (20 mL). The organic layer was separated, and the aqueous layer was extracted with EtOAc (20 mL×3). The combined organic layer was washed with water (20 mL×3), brine (20 mL×3), dried over Na₂SO₄, filtered, and concentrated under reduced pressure. The residue was purified by flash column chromatography (100% ethyl acetate then 0-8% MeOH in CH₂Cl₂) to afford tert-butyl 3-[7-(8-ethyl-1-naphthyl)-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (547 mg, 61%) as a yellow solid. MS (ESI) m/z: 657.1 [M+H]⁺.

Step 3: Preparation of tert-butyl 3-[7-(8-ethyl-1-naphthyl)-8-fluoro-2-[[(2S,5S)-5-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate

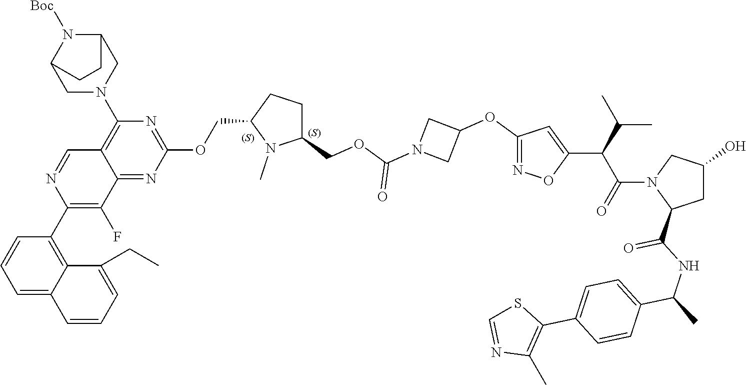

To a reaction mixture of tert-butyl 3-[7-(8-ethyl-1-naphthyl)-8-fluoro-2-[[(2S,5S)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (220 mg, 313 umol) in THF (10 mL) was added TEA (220 uL, 1.6 mmol), DMAP (7.6 mg, 63 umol) and (4-nitrophenyl) carbonochloridate (120 mg, 595 umol), the resulting suspension was stirred at 25° C. for 15 h. Then (2S,4R)-1-[(2R)-2-[3-(azetidin-3-yloxy)isoxazol-5-yl]-3-methyl-butanoyl]-4-hydroxy-N-[(1S)-1-[4-(4-methylthiazol-5-yl)phenyl]ethyl]pyrrolidine-2-carboxamide (260 mg, 469 umol) was added, the resulting suspension was stirred at 25° C. for 2 h. The reaction mixture was filtered and concentrated under reduced pressure. The residue was purified by column chromatography (0-6% MeOH in CH₂Cl₂) to afford tert-butyl 3-[7-(8-ethyl-1-naphthyl)-8-fluoro-2-[[(2S,5S)-5-[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate (546 mg, 99%) as a yellow oil. MS (ESI) m/z: 1236.4 [M+H]⁺.

Step 4: Preparation of [(2S,5S)-5-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1-methyl-pyrrolidin-2-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate

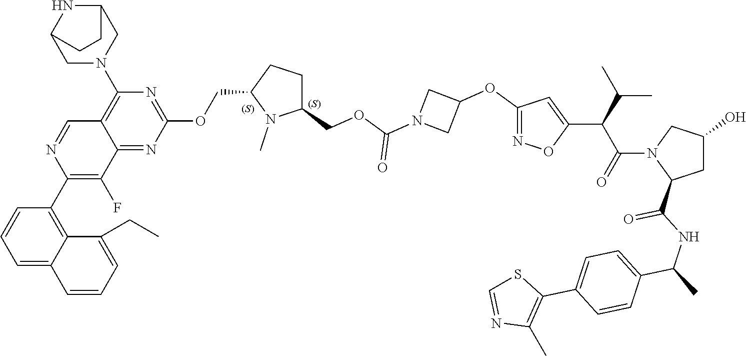

To a reaction mixture of tert-butyl 3-[7-(8-ethyl-1-naphthyl)-8-fluoro-2-[[(2S,5S)-5-[[3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate (546 mg, 309 umol) in CH₂Cl₂ (6 mL) was added TFA (3 mL, 40 mmol), the resulting suspension was stirred at 25° C. for 0.5 h, then concentrated under reduced pressure. The pH of the residue was adjusted to 8 by sat. aq. NaHCO₃. The mixture was filtered, washed with water (10 mL) and dried under reduced pressure. The crude product was purified by prep-HPLC (column: Xtimate C18 150*40 mm*10 um; mobile phase: [water (formic acid)-acetonitrile]; Begin B: 0; End B: 38; Gradient Time (min): 36; 100% B Hold Time (min): 3; Flow Rate (ml/min): 60) to afford [(2S,5S)-5-[[4-(3,8-diazabicyclo[3.2.1]octan-3-yl)-7-(8-ethyl-1-naphthyl)-8-fluoro-pyrido[4,3-d]pyrimidin-2-yl]oxymethyl]-1-methyl-pyrrolidin-2-yl]methyl 3-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxyazetidine-1-carboxylate (151.1 mg, 41%, formic acid salt) as a white solid. MS (ESI) m/z: 1136.5 [M+H]⁺; ¹H NMR (400 MHz, DMSO-d₆) δ 9.11-9.08 (m, 1H), 8.98 (s, 1H), 8.42 (d, J=7.6 Hz, 1H), 8.07 (d, J=8.0 Hz, 1H), 7.91 (d, J=8.0 Hz, 1H), 7.58 (t, J=7.6 Hz, 1H), 7.51 (t, J=7.6 Hz, 1H), 7.46-7.41 (m, 3H), 7.40-7.34 (m, 3H), 6.15 (s, 1H), 5.18-5.10 (m, 1H), 4.95-4.86 (m, 1H), 4.50-4.34 (m, 4H), 4.31-4.19 (m, 4H), 3.98 (br d, J=4.4 Hz, 2H), 3.90 (br d, J=5.2 Hz, 2H), 3.72-3.51 (m, 10H), 2.48-2.43 (m, 6H), 2.42-2.12 (m, 4H), 2.06-1.90 (m, 3H), 1.77 (ddd, J=4.8, 8.0, 12.8 Hz, 1H), 1.72-1.54 (m, 6H), 1.45-1.34 (m, 3H), 0.99-0.92 (m, 3H), 0.87-0.76 (m, 6H).

Exemplary Synthesis of (2S,4R)-1-[(2S)-2-{3-[(2R)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-2-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 224)

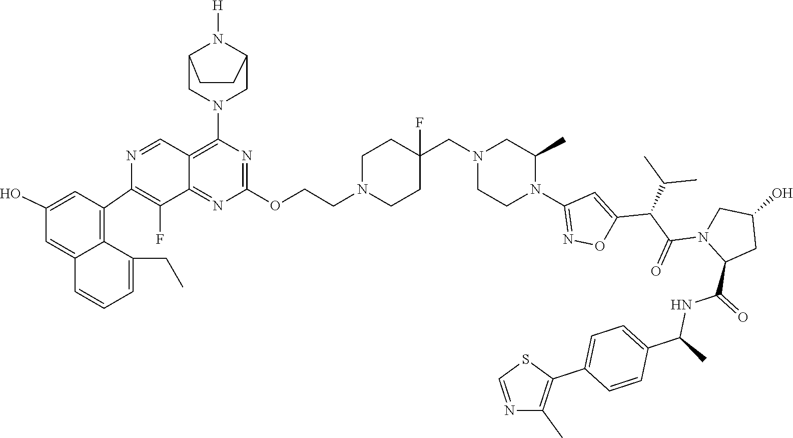 [SMILES: CCc1cccc2cc(O)cc(-c3ncc4c(N5CC6CCC(C5)N6)nc(OCCN5CCC(F)(CN6CCN(c7cc([C@@H](C(=O)N8C[C@H](O)C[C@H]8C(=O)N[C@@H](C)c8ccc(-c9scnc9C)cc8)C(C)C)on7)[C@H](C)C6)CC5)nc4c3F)c12]

The title compound was prepared analogously to (2S,4R)-1-[(2R)-2-{3-[(2R)-4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methyl]-2-methylpiperazin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide, starting from (2S,4R)-4-hydroxy-1-[(2S)-3-methyl-2-[3-[(2R)-2-methylpiperazin-1-yl]isoxazol-5-yl]butanoyl]-N-[(1S)-1-[4-(4-methylthiazol-5-yl)phenyl]ethyl]pyrrolidine-2-carboxamide and tert-butyl 4-fluoro-4-formyl-piperidine-1-carboxylate. (white solid). MS (ESI) m/z: 1168.3 [M+H]⁺; ¹H NMR (400 MHz, CD₃OD) δ 9.04 (s, 1H), 8.87 (s, 1H), 7.62 (d, J=8.4 Hz, 1H), 7.47-7.32 (m, 5H), 7.29 (t, J=2.0 Hz, 1H), 7.15 (d, J=7.2 Hz, 1H), 7.01 (d, J=2.4 Hz, 1H), 6.09 (s, 1H), 5.02-4.96 (m, 1H), 4.69-4.56 (m, 9H), 4.43 (s, 1H), 3.77-3.66 (m, 8H), 2.95-2.85 (m, 5H), 2.57-2.50 (m, 2H), 2.49 (s, 1H), 2.47 (s, 3H), 2.40-2.21 (m, 6H), 2.02-1.75 (m, 9H), 1.48 (d, J=7.2 Hz, 3H), 1.19 (d, J=6.4 Hz, 3H), 1.06 (d, J=6.4 Hz, 3H), 0.93-0.86 (m, 6H).

Exemplary Synthesis of [(2R,5S)-5-{[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]methyl}-1-methylpyrrolidin-2-yl]methyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazine-1-carboxylate (Compound 259)

Step 1: Preparation of tert-butyl(5S)-2-hydroxy-5-(hydroxymethyl)pyrrolidine-1-carboxylate

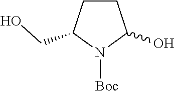 [SMILES: CC(C)(C)OC(=O)N1C(O)CC[C@H]1CO]

To a reaction mixture of O1-tert-butyl O2-methyl (2S)-5-oxopyrrolidine-1,2-dicarboxylate (36.0 g, 148 mmol) in THF (700 mL) was added diisobutylalumane (1.0 M, 500 mL) at −78° C., the resulting suspension was gradually warmed up to 0° C. and further stirred for 2 h. The reaction mixture was quenched with water (20 mL), 15% aqueous NaOH solution (20 mL) and water (50 mL) at 0° C. The mixture was warmed to 20° C. and stirred at 20° C. for 15 min, then it was dried over Na₂SO₄, filtered and concentrated under reduced pressure to afford tert-butyl (5S)-2-hydroxy-5-(hydroxymethyl)pyrrolidine-1-carboxylate (32.15 g, crude) as a colorless oil, which was used in the next step directly.

Step 2: Preparation of tert-butyl(2S)-2-(hydroxymethyl)-5-methoxy-pyrrolidine-1-carboxylate

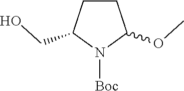 [SMILES: COC1CC[C@@H](CO)N1C(=O)OC(C)(C)C]

To a reaction mixture of tert-butyl(5S)-2-hydroxy-5-(hydroxymethyl)pyrrolidine-1-carboxylate (32.15 g, 148 mmol) in MeOH (400 mL) was added PPTS (3.72 g, 15 mmol), the resulting suspension was stirred at 20° C. for 15 h. The reaction mixture was diluted with EtOAc (1500 mL) and washed with aqueous HCl (0.1 M, 500 mL), sat. aq. NaHCO₃ (500 mL) and brine (500 mL). The organic layer was dried over Na₂SO₄, filtered, and concentrated under reduced pressure. The residue was purified by flash column (0-15% then up to 25% ethyl acetate in petroleum ether) to afford tert-butyl(2S)-2-(hydroxymethyl)-5-methoxy-pyrrolidine-1-carboxylate (25.25 g, 70%) as a colorless oil. ¹H NMR (400 MHz, CDCl₃-d) δ 5.33-5.10 (m, 1H), 4.09-3.95 (m, 1H), 3.76 (br d, J=10.8 Hz, 1H), 3.54 (br dd, J=6.4, 11.2 Hz, 1H), 3.44-3.25 (m, 3H), 2.07-2.02 (m, 1H), 1.93-1.75 (m, 3H), 1.50 (s, 9H).

Step 3: Preparation of tert-butyl(2S)-2-(benzyloxymethyl)-5-methoxy-pyrrolidine-1-carboxylate

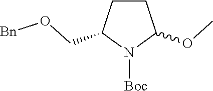 [SMILES: COC1CC[C@@H](COCc2ccccc2)N1C(=O)OC(C)(C)C]

To a mixture of tert-butyl(2S)-2-(hydroxymethyl)-5-methoxy-pyrrolidine-1-carboxylate (25.25 g, 104 mmol) in DMF (250 mL) was added NaH (4.99 g, 60%, 125 mmol) at 0° C. under N₂ atmosphere. After 30 min, BnBr (14.8 mL, 124 mmol) was added dropwise, the resulting suspension was stirred at 20° C. for 15 h. The reaction mixture was quenched with aqueous saturated NH₄Cl (250 mL) at 0° C. and diluted with EtOAc (500 mL). The organic layer was separated, the aqueous layer was extracted with EtOAc (500 mL×3). The combined organic layer was washed with water (500 mL×3), brine (500 mL×3), the solvent was dried over Na₂SO₄, filtered, and concentrated under reduced pressure. The residue was purified by flash column (0-10% ethyl acetate in petroleum ether) to afford tert-butyl(2S)-2-(benzyloxymethyl)-5-methoxy-pyrrolidine-1-carboxylate (26.74 g, 76%) as a colorless oil. ¹H NMR (400 MHz, CDCl₃-d) δ 7.38-7.27 (m, 5H), 5.56-4.82 (m, 1H), 4.62-4.48 (m, 2H), 4.11-3.73 (m, 2H), 3.67-3.47 (m, 1H), 3.40-3.26 (m, 3H), 2.24-2.10 (m, 1H), 2.01-1.71 (m, 3H), 1.51-1.41 (m, 9H).

Step 4: Preparation of tert-butyl(2S,5R)-2-(benzyloxymethyl)-5-cyano-pyrrolidine-1-carboxylate and tert-butyl(2S,5S)-2-(benzyloxymethyl)-5-cyano-pyrrolidine-1-carboxylate

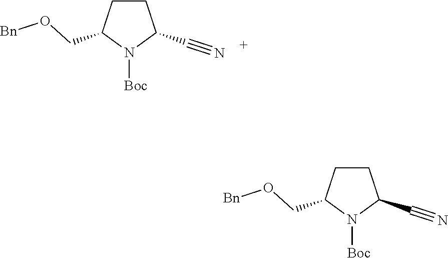 [SMILES: CC(C)(C)OC(=O)N1[C@H](COCc2ccccc2)CC[C@@H]1C#N ; CC(C)(C)OC(=O)N1[C@H](COCc2ccccc2)CC[C@H]1C#N]

To a reaction mixture of tert-butyl(2S)-2-(benzyloxymethyl)-5-methoxy-pyrrolidine-1-carboxylate (26.74 g, 79 mmol) in CH₂Cl₂ (500 mL) was added TMSCN (14.35 mL, 115 mmol) and BF₃·Et₂O (14.15 mL, 115 mmol) at −78° C. under N₂ atmosphere, the resulting suspension was stirred at −78° C. for 1 h. The reaction mixture was quenched with sat. aq. NaHCO₃ (250 mL), extracted with CH₂Cl₂ (250 mL×3). The combined organic layer was dried over Na₂SO₄, filtered, and concentrated under reduced pressure. The residue was purified by flash column (0-10% ethyl acetate in petroleum ether) to afford tert-butyl(2S,5S)-2-(benzyloxymethyl)-5-cyano-pyrrolidine-1-carboxylate (16.31 g, 62%) as a colorless oil. ¹H NMR (400 MHz, CDCl₃-d) δ 7.39-7.27 (m, 5H), 4.57-4.41 (m, 3H), 4.16-3.94 (m, 1H), 3.64-3.40 (m, 2H), 2.45-2.08 (m, 4H), 1.54-1.42 (m, 9H). Tert-butyl(2S,5R)-2-(benzyloxymethyl)-5-cyano-pyrrolidine-1-carboxylate (7.78 g, 30%) was obtained as a colorless oil. ¹H NMR (400 MHz, CDCl₃-d) δ 7.40-7.27 (m, 5H), 4.65-4.41 (m, 3H), 4.13-3.91 (m, 1H), 3.70-3.43 (m, 2H), 2.41-2.00 (m, 4H), 1.56-1.40 (m, 9H).

Step 5: Preparation of tert-butyl(2S,5R)-2-(benzyloxymethyl)-5-formyl-pyrrolidine-1-carboxylate

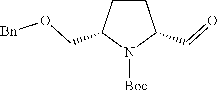 [SMILES: CC(C)(C)OC(=O)N1[C@H](COCc2ccccc2)CC[C@@H]1C=O]

To a reaction mixture of tert-butyl(2S,5R)-2-(benzyloxymethyl)-5-cyano-pyrrolidine-1-carboxylate (500 mg, 1.5 mmol) in toluene (5 mL) was added diisobutylalumane (1.0 M, 2.25 mL) at −78° C., the resulting suspension was stirred at −78° C. for 1 h. The reaction mixture was quenched with water (90 uL), 15% NaOH aqueous solution (90 uL) and water (0.25 mL) at −78° C. The mixture was warmed to 20° C. and stirred at 20° C. for 15 min, then it was dried over Na₂SO₄, filtered, and washed with EtOAc (30 mL). The filtrate was washed with water (20 mL×2), brine (20 mL), dried over Na₂SO₄, filtered, and concentrated under reduced pressure. The residue was purified by flash column (0-10% ethyl acetate in petroleum ether) to afford tert-butyl(2S,5R)-2-(benzyloxymethyl)-5-formyl-pyrrolidine-1-carboxylate (236 mg, 47%) as a colorless oil. MS (ESI) m/z: 220.1 [M+H−100]⁺.

Step 6: Preparation of tert-butyl(2S,5R)-2-(benzyloxymethyl)-5-(hydroxymethyl)pyrrolidine-1-carboxylate

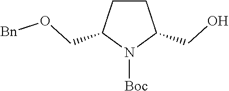 [SMILES: CC(C)(C)OC(=O)N1[C@@H](CO)CC[C@H]1COCc1ccccc1]

To a reaction mixture of tert-butyl(2S,5R)-2-(benzyloxymethyl)-5-formyl-pyrrolidine-1-carboxylate (420 mg, 1.3 mmol) in MeOH (10 mL) was added K₂CO₃ (864 mg, 6.3 mmol), the resulting suspension was stirred at 70° C. for 2 h. Then the reaction mixture was cooled to room temperature (20° C.), NaBH₄ (47.2 mg, 1.3 mmol) was added. The reaction mixture was stirred at 20° C. for 0.5 h. The pH of the reaction mixture was adjusted to 7 with HCl (2 M) and extracted with EtOAc (20 mL×3). The combined organic layer was washed with brine (20 mL×3) and dried over Na₂SO₄, filtered, and concentrated under reduced pressure. The residue was purified by flash column (0-10% ethyl acetate in petroleum ether) to afford tert-butyl(2S,5R)-2-(benzyloxymethyl)-5-(hydroxymethyl)pyrrolidine-1-carboxylate (255 mg, 63%) as a colorless oil. MS (ESI) m/z: 344.1 [M+Na]⁺.

Step 7: Preparation of [(2R,5S)-5-(benzyloxymethyl)-1-methyl-pyrrolidin-2-yl]methanol

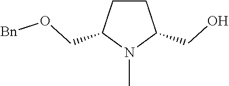 [SMILES: CN1[C@@H](CO)CC[C@H]1COCc1ccccc1]

To a mixture of LiAlH₄ (312 mg, 8 mmol) in THF (5 mL) was dropwise added a solution of tert-butyl(2S,5R)-2-(benzyloxymethyl)-5-(hydroxymethyl)pyrrolidine-1-carboxylate (660 mg, 2 mmol) in THF (10 mL) at 0° C., the resulting suspension was stirred at 70° C. for 15 h. The reaction mixture was cooled to 0° C. and quenched with water (312 uL), 15% NaOH solution (312 uL) and water (936 uL). The mixture was dried over Na₂SO₄, filtered, and concentrated under reduced pressure to afford [(2R,5S)-5-(benzyloxymethyl)-1-methyl-pyrrolidin-2-yl]methanol (483 mg, crude) as a colorless oil, which was used in the next step directly. MS (ESI) m/z: 236.1 [M+H]⁺.

Step 8: Preparation of [(2R,5S)-5-(benzyloxymethyl)-1-methyl-pyrrolidin-2-yl]methoxy-tert-butyl-diphenyl-silane

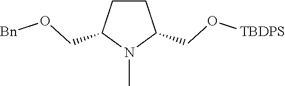

To a reaction mixture of [(2R,5S)-5-(benzyloxymethyl)-1-methyl-pyrrolidin-2-yl]methanol (483 mg, 2 mmol) in CH₂Cl₂ (10 mL) were added imidazole (168 mg, 2.5 mmol) and TBDPSCl (582 uL, 2 mmol) at 20° C., the resulting suspension was stirred at 20° C. for 15 h. The reaction mixture was filtered, the filtrate solution was washed with water (10 mL×3), brine (10 mL×3), dried over Na₂SO₄, filtered, and concentrated under reduced pressure. The residue was purified by flash column (0-5% ethyl acetate in petroleum ether) to afford [(2R,5S)-5-(benzyloxymethyl)-1-methyl-pyrrolidin-2-yl]methoxy-tert-butyl-diphenyl-silane (684 mg, 65%) as a colorless oil. MS (ESI) m/z: 474.0 [M+H]⁺.

Step 9: Preparation of [(2S,5R)-5-[[tert-butyl(diphenyl)silyl]oxymethyl]-1-methyl-pyrrolidin-2-yl]methanol

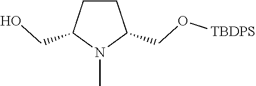

To a solution of [(2R,5S)-5-(benzyloxymethyl)-1-methyl-pyrrolidin-2-yl]methoxy-tert-butyl-diphenyl-silane (384 mg, 754 umol) in CH₂Cl₂ (6 mL) was added a solution of BBr₃ (145 uL, 1.5 mmol) under N₂ at −78° C. The resulting suspension was stirred under N₂ at −78° C. for 2 h. The reaction mixture was quenched with water (10 mL), concentrated under reduced pressure. The residue was extracted with MTBE (10 mL×3), the aqueous layer was adjusted with sat. NaHCO₃ to pH 8. The aqueous layer was extracted with CH₂Cl₂/MeOH (10 mL×3, V/V=10:1). The combined organic layer was washed with brine (10 mL×2), dried over Na₂SO₄, filtered, and concentrated under reduced pressure. The residue was purified by flash column (0-2% MeOH in CH₂Cl₂) to afford [(2S,5R)-5-[[tert-butyl(diphenyl)silyl]oxymethyl]-1-methyl-pyrrolidin-2-yl]methanol (105 mg, 34%) as a colorless oil. MS (ESI) m/z: 384.0 [M+H]⁺.

Step 10: Preparation of tert-butyl 3-[2-[[(2S,5R)-5-[[tert-butyl(diphenyl)silyl]oxymethyl]-1-methyl-pyrrolidin-2-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate

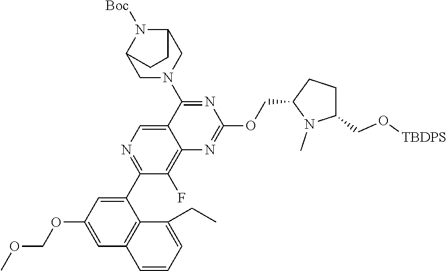

To a reaction mixture of tert-butyl 3-[7-[8-ethyl-3-(methoxymethoxy)-1-naphthyl]-8-fluoro-2-methylsulfonyl-pyrido[4,3-d]pyrimidin-4-yl]-3,8-diazabicyclo[3.2.1]octane-8-carboxylate (170 mg, 261 umol) and [(2S,5R)-5-[[tert-butyl(diphenyl)silyl]oxymethyl]-1-methyl-pyrrolidin-2-yl]methanol (105 mg, 260 umol) in dioxane (4 mL) were added 4 A MS (300 mg). Then cooled to 0° C., t-BuONa (75.20 mg, 782 umol) was added. The resulting suspension was stirred at 0° C. for 0.5 h. The reaction mixture was quenched with HCl (2 M) to pH 6. The mixture was dried over Na₂SO₄, filtered, and the cake was washed with EtOAc (10 mL). The filtrate was washed with water (10 mL×2), brine (10 mL×2), dried over Na₂SO₄, filtered, and concentrated under reduced pressure. The residue was purified by flash column (0-30% ethyl acetate in petroleum ether) to afford tert-butyl 3-[2-[[(2S,5R)-5-[[tert-butyl(diphenyl)silyl]oxymethyl]-1-methyl-pyrrolidin-2-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (158 mg, 59%) as a colorless oil. MS (ESI) m/z: 955.2 [M+H]⁺.

Step 11: Preparation of tert-butyl 3-[7-[8-ethyl-3-(methoxymethoxy)-1-naphthyl]-8-fluoro-2-[[(2S,5R)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate

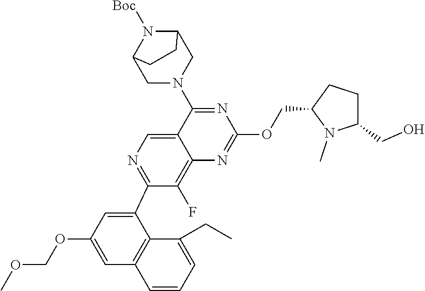 [SMILES: CCc1cccc2cc(OCOC)cc(-c3ncc4c(N5CC6CCC(C5)N6C(=O)OC(C)(C)C)nc(OC[C@@H]5CC[C@H](CO)N5C)nc4c3F)c12]

To a reaction mixture of tert-butyl 3-[2-[[(2S,5R)-5-[[tert-butyl(diphenyl)silyl]oxymethyl]-1-methyl-pyrrolidin-2-yl]methoxy]-7-[8-ethyl-3-(methoxymethoxy)-1-naphthyl]-8-fluoro-pyrido[4,3-d]pyrimidin-4-yl]-3,8-diazabicyclo[3.2.1]octane-8-carboxylate (158 mg, 155 umol) in THF (5 mL) was added TBAF (1.0 M, 310 uL), the resulting suspension was stirred at 25° C. for 1 h. The reaction mixture was concentrated under reduced pressure to afford tert-butyl 3-[7-[8-ethyl-3-(methoxymethoxy)-1-naphthyl]-8-fluoro-2-[[(2S,5R)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (111.2 mg, crude) as a yellow oil, which was used in the next step directly. MS (ESI) m/z: 717.1 [M+H]⁺.

Step 12: Preparation of tert-butyl 3-[7-[8-ethyl-3-(methoxymethoxy)-1-naphthyl]-8-fluoro-2-[[(2S,5R)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate

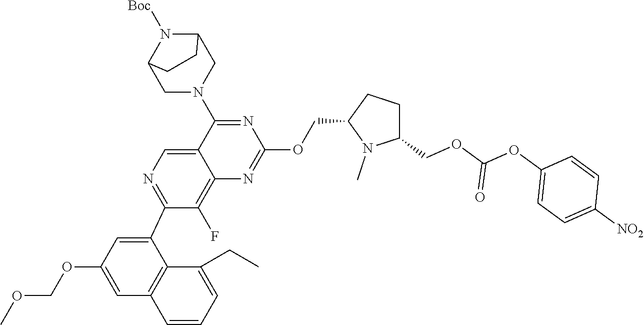 [SMILES: CCc1cccc2cc(OCOC)cc(-c3ncc4c(N5CC6CCC(C5)N6C(=O)OC(C)(C)C)nc(OC[C@@H]5CC[C@H](COC(=O)Oc6ccc([N+](=O)[O-])cc6)N5C)nc4c3F)c12]

To a reaction mixture of tert-butyl 3-[7-[8-ethyl-3-(methoxymethoxy)-1-naphthyl]-8-fluoro-2-[[(2S,5R)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (111 mg, 155 umol) in THF (10 mL) was added TEA (108 uL, 779 umol), DMAP (6 mg, 46 umol) and (4-nitrophenyl) carbonochloridate (59 mg, 294 umol), the resulting suspension was stirred at 25° C. for 15 h. The reaction mixture was filtered and concentrated under reduced pressure. The residue was purified by flash column (100% ethyl acetate to 0-4% MeOH in CH₂Cl₂) to afford tert-butyl 3-[7-[8-ethyl-3-(methoxymethoxy)-1-naphthyl]-8-fluoro-2-[[(2S,5R)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (100 mg, 72%) as a yellow oil. MS (ESI) m/z: 717.1 [M+H]⁺.

Step 13: Preparation of tert-butyl 3-[7-[8-ethyl-3-(methoxymethoxy)-1-naphthyl]-8-fluoro-2-[[(2S,5R)-5-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate

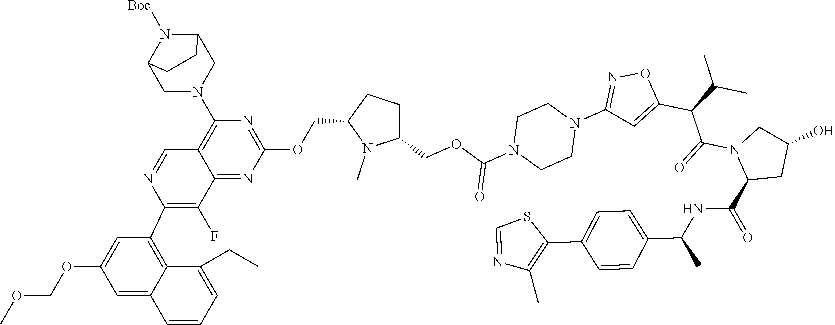 [SMILES: CCc1cccc2cc(OCOC)cc(-c3ncc4c(N5CC6CCC(C5)N6C(=O)OC(C)(C)C)nc(OC[C@@H]5CC[C@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12]

To a mixture of tert-butyl 3-[7-[8-ethyl-3-(methoxymethoxy)-1-naphthyl]-8-fluoro-2-[[(2S,5R)-5-(hydroxymethyl)-1-methyl-pyrrolidin-2-yl]methoxy]pyrido[4,3-d]pyrimidin-4-yl]-3,8-diazabicyclo[3.2.1]octane-8-carboxylate (100 mg, 112 umol) in THF (5 mL) was added TEA (78 uL, 560 umol), DMAP (4 mg, 33 umol) and (4-nitrophenyl) carbonochloridate (43 mg, 213 umol), the resulting suspension was stirred at 30° C. for 15 h. Then (2S,4R)-4-hydroxy-1-[(2R)-3-methyl-2-(3-piperazin-1-ylisoxazol-5-yl)butanoyl]-N-[(1S)-1-[4-(4-methylthiazol-5-yl)phenyl]ethyl]pyrrolidine-2-carboxamide (127 mg, 224 umol) was added, the resulting suspension was stirred at 30° C. for 2 h. The reaction mixture was filtered and concentrated under reduced pressure. The residue was purified by flash column (0-4% MeOH in CH₂Cl₂) to afford tert-butyl 3-[7-[8-ethyl-3-(methoxymethoxy)-1-naphthyl]-8-fluoro-2-[[(2S,5R)-5-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate (120 mg, 69%) as a yellow solid. MS (ESI) m/z: 1309.3 [M+H]⁺.

Step 14: Preparation of [(2R,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate

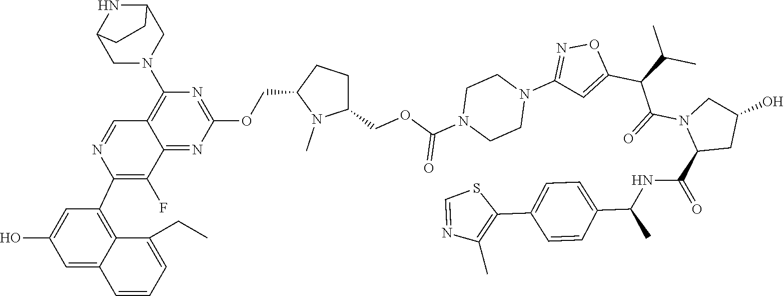 [SMILES: CCc1cccc2cc(O)cc(-c3ncc4c(N5CC6CCC(C5)N6)nc(OC[C@@H]5CC[C@H](COC(=O)N6CCN(c7cc([C@H](C(=O)N8C[C@H](O)C[C@H]8C(=O)N[C@@H](C)c8ccc(-c9scnc9C)cc8)C(C)C)on7)CC6)N5C)nc4c3F)c12]

To a reaction mixture of tert-butyl 3-[7-[8-ethyl-3-(methoxymethoxy)-1-naphthyl]-8-fluoro-2-[[(2S,5R)-5-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carbonyl]oxymethyl]-1-methyl-pyrrolidin-2-yl]methoxy]pyrido[4,3-d]pyrimidin-4-yl]-3,8-diazabicyclo[3.2.1]octane-8-carboxylate (120 mg, 77 umol) in CH₂Cl₂ (2 mL) was added HCl in dioxane (4.0 M, 1 mL), the resulting suspension was stirred at 20° C. for 0.25 h. The reaction mixture was diluted with petroleum ether (30 mL), the precipitate was collected, dissolved with THF (15 mL), adjusted the pH with triethylamine to 8. The mixture was filtered, concentrated under reduced pressure. The crude product was purified by prep-HPLC (column: Xtimate C18 150*40 mm*10 um; mobile phase: [water (formic acid)-acetonitrile]; Begin B: 0 End B: 36; Gradient Time (min): 36; 100% B Hold Time (min): 2; Flow Rate (ml/min): 60) to afford [(2R,5S)-5-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxymethyl]-1-methyl-pyrrolidin-2-yl]methyl 4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]piperazine-1-carboxylate (53.4 mg, 57%) as a white solid. MS (ESI) m/z: 1165.6 [M+H]; ¹H NMR (400 MHz, DMSO-d₆) δ 9.90 (br d, J=1.6 Hz, 1H), 9.08 (s, 1H), 8.98 (s, 1H), 8.40 (br d, J=7.6 Hz, 1H), 7.66 (d, J=8.4 Hz, 1H), 7.46-7.39 (m, 2H), 7.39-7.31 (m, 3H), 7.28 (d, J=2.4 Hz, 1H), 7.11 (d, J=6.4 Hz, 1H), 6.97 (d, J=1.6 Hz, 1H), 6.19-6.03 (m, 1H), 4.97-4.86 (m, 1H), 4.49-4.31 (m, 4H), 4.30-4.25 (m, 1H), 4.20-4.12 (m, 1H), 4.02-3.90 (m, 2H), 3.71 (br dd, J=4.0, 9.6 Hz, 1H), 3.67-3.49 (m, 6H), 3.45 (br s, 6H), 3.17 (br s, 4H), 2.87-2.81 (m, 1H), 2.72-2.65 (m, 1H), 2.47-2.42 (m, 6H), 2.28-2.14 (m, 3H), 2.06-1.97 (m, 1H), 1.96-1.84 (m, 2H), 1.82-1.73 (m, 1H), 1.71-1.53 (m, 6H), 1.47-1.33 (m, 3H), 0.94 (br d, J=6.4 Hz, 3H), 0.87-0.73 (m, 6H).

Exemplary Synthesis of (2S,4R)-1-[(2S)-2-(2-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]-4-fluoropiperidin-1-yl}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide (Compound 260)

Step 1: Preparation of 4-[(1-benzyloxycarbonyl-4-piperidyl)methyl]-4-hydroxy-piperidine-1-carboxylate

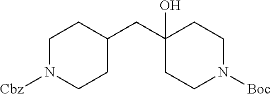 [SMILES: CC(C)(C)OC(=O)N1CCC(O)(CC2CCN(C(=O)OCc3ccccc3)CC2)CC1]

To a stirred solution of tert-butyl 4-hydroxy-4-(4-piperidylmethyl)piperidine-1-carboxylate (6.0 g, 20 mmol) in ethyl alcohol (100 mL) was added NaHCO₃ (10.13 g, 120 mmol) in H₂O (100 mL), and CbzCl (3.43 mL, 24 mmol) at 0° C., the reaction mixture was allowed to warmed to 25° C. and stirred for 16 h. The reaction mixture was extracted with ethyl acetate (250 mL×2), dried over Na₂SO₄, filtered, and concentrated under reduced pressure. The residue was purified by flash column (0-22% tetrahydrofuran in petroleum ether) to afford tert-butyl 4-[(1-benzyloxycarbonyl-4-piperidyl)methyl]-4-hydroxy-piperidine-1-carboxylate (7.25 g, 73%) as a yellow oil. MS (ESI) m/z: 455.2 [M+23]⁺.

Step 2: Preparation of tert-butyl 4-[(1-benzyloxycarbonyl-4-piperidyl)methyl]-4-fluoro-piperdine-1-carboxylate

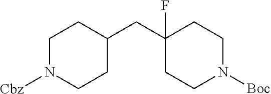 [SMILES: CC(C)(C)OC(=O)N1CCC(F)(CC2CCN(C(=O)OCc3ccccc3)CC2)CC1]

To a solution of tert-butyl 4-[(1-benzyloxycarbonyl-4-piperidyl)methyl]-4-hydroxy-piperidine-1-carboxylate (7.4 g, 17 mmol) in CH₂Cl₂ (250 mL) was added DAST (2.71 mL, 20 mmol) slowly at 0° C., the reaction mixture was allowed to warmed to 25° C. and stirred for 2 h. The mixture was concentrated, quenched with sat. NaHCO₃ to pH 8, extracted with dichloromethane (50 mL×3). The combined organic phase was washed with brine (20 mL), dried over anhydrous sodium sulfate, filtered, and concentrated. The residue was purified by flash column (0-11% tetrahydrofuran in petroleum ether) to afford tert-butyl 4-[(1-benzyloxycarbonyl-4-piperidyl)methyl]-4-fluoro-piperidine-1-carboxylate (4.6 g, 62%) as a colorless solid. MS (ESI) m/z: 457.2 [M+23]⁺.

Step 3: Preparation of benzyl 4-[(4-fluoro-4-piperidyl)methyl]piperidine-1-carboxylate

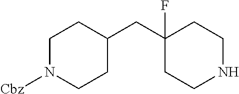 [SMILES: O=C(OCc1ccccc1)N1CCC(CC2(F)CCNCC2)CC1]

To a solution of tert-butyl 4-[(1-benzyloxycarbonyl-4-piperidyl)methyl]-4-fluoro-piperidine-1-carboxylate (1.0 g, 2 mmol) in dichloromethane (10 mL) was added HCl in dioxane (4 M, 8.1 mL). The mixture was stirred at 25° C. for 30 min. The mixture was concentrated, quenched with sat. NaHCO₃ to pH 8, extracted with dichloromethane (60 mL×3). The combined organic phase was washed with brine (20 mL), dried over anhydrous sodium sulfate, filtered, and concentrated to give benzyl 4-[(4-fluoro-4-piperidyl)methyl]piperidine-1-carboxylate (750 mg, 97%) as a colorless oil, which was used directly in the next step. MS (ESI) m/z: 335.2 [M+1]⁺.

Step 4: Preparation of benzyl 4-[[1-(2-ethoxy-2-oxo-ethyl)-4-fluoro-4-piperidyl]methyl]piperidine-1-carboxylate

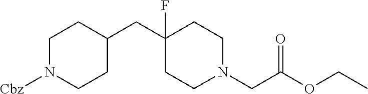 [SMILES: CCOC(=O)CN1CCC(F)(CC2CCN(C(=O)OCc3ccccc3)CC2)CC1]

To a solution of benzyl 4-[(4-fluoro-4-piperidyl)methyl]piperidine-1-carboxylate (1.2 g, 3.6 mmol) in tetrahydrofuran (20 mL) were added DIEA (1.3 mL, 7 mmol) and ethyl 2-bromoacetate (0.39 mL, 3.6 mmol). The mixture was stirred at 25° C. for 16 h. The reaction was quenched by water (25 mL) and extracted with ethyl acetate (50 mL×3). The combined organic layer was washed with brine (50 mL), dried over anhydrous Na₂SO₄, filtered, and concentrated under reduced pressure. The crude product was purified by flash column (0-35% tetrahydrofuran in petroleum ether) to give benzyl 4-[[1-(2-ethoxy-2-oxo-ethyl)-4-fluoro-4-piperidyl]methyl]piperidine-1-carboxylate (1.1 g, 74%) as a yellow oil. MS (ESI) m/z: 421.3 [M+1]⁺.

Step 5: Preparation of 2-[[4-[(1-benzyloxycarbonyl-4-piperidyl)methyl]-4-fluoro-1-piperidyl]acetic acid

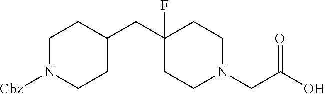 [SMILES: O=C(O)CN1CCC(F)(CC2CCN(C(=O)OCc3ccccc3)CC2)CC1]

To a solution of benzyl 4-[[1-(2-ethoxy-2-oxo-ethyl)-4-fluoro-4-piperidyl]methyl]piperidine-1-carboxylate (500 mg, 1.2 mmol) in tetrahydrofuran (20 mL) and H₂O (5 mL) was added LiOH. H₂O (249.5 mg, 6 mmol). The reaction mixture was stirred at 25° C. for 16 h. The mixture was diluted with H₂O (20 mL), adjusted the pH to 4 with 2 N hydrochloric acid solution. The mixture was concentrated and lyophilized to give 2-[[4-[(1-benzyloxycarbonyl-4-piperidyl)methyl]-4-fluoro-1-piperidyl]acetic acid (460 mg, 84%) as a white solid. MS (ESI) m/z: 393.2 [M+1]⁺.

Step 6: Preparation of 4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]piperidine-1-carboxylate

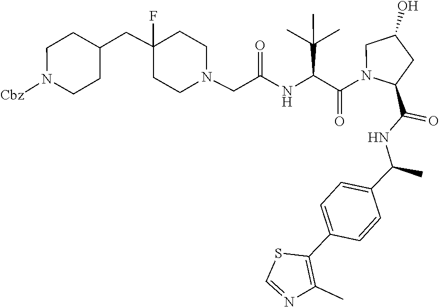 [SMILES: Cc1ncsc1-c1ccc([C@H](C)NC(=O)[C@@H]2C[C@@H](O)CN2C(=O)[C@@H](NC(=O)CN2CCC(F)(CC3CCN(C(=O)OCc4ccccc4)CC3)CC2)C(C)(C)C)cc1]

To a solution of 2-[[4-[(1-benzyloxycarbonyl-4-piperidyl)methyl]-4-fluoro-1-piperidyl]acetic acid (460 mg, 1.2 mmol), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (573 mg, 1.2 mmol) and DIEA (2.0 mL, 12 mmol) in dichloromethane (5.0 mL) was added HATU (490 mg, 1.3 mmol). The mixture was stirred at 25° C. for 3 h. The reaction mixture was diluted with water (40 mL), extracted with dichloromethane (30 mL×3), the combined organic phase was washed with brine (20 mL), dried over anhydrous sodium sulfate, filtered, and concentrated in vacuum. The residue was purified by flash column (0-6% methanol in dichloromethane) to afford benzyl 4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]piperidine-1-carboxylate (420 mg, 34%) as a yellow solid. MS (ESI) m/z: 819.4 [M+1]⁺.

Step 7: Preparation of (2S,4R)-1-[(2S)-2-[[2-[[4-fluoro-4-(4-piperidylmethyl)-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

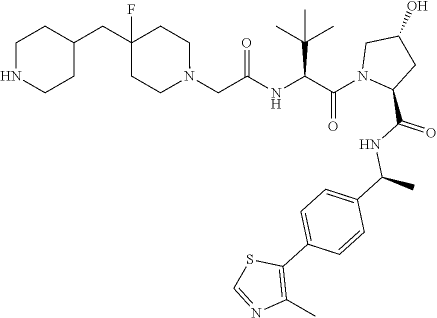 [SMILES: Cc1ncsc1-c1ccc([C@H](C)NC(=O)[C@@H]2C[C@@H](O)CN2C(=O)[C@@H](NC(=O)CN2CCC(F)(CC3CCNCC3)CC2)C(C)(C)C)cc1]

To a solution of benzyl 4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]piperidine-1-carboxylate (400 mg, 0.5 mmol) in TFA (7.2 mL, 98 mmol), and the reaction mixture was stirred at 70° C. for 2 h. The mixture was basified with saturated NaHCO₃ solution until pH 8-9 and extracted with dichloromethane/methyl alcohol (50 mL×3, v/v=10/1). The combined extracts were washed with brine (5 mL), dried over anhydrous Na₂SO₄, filtered and concentrated under reduced pressure to give (2S,4R)-1-[(2S)-2-[2-[[4-fluoro-4-(4-piperidylmethyl)-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (330 mg, 65%) as a yellow solid. MS (ESI) m/z: 685.5 [M+1]⁺.

Step 8: Preparation of tert-butyl 3-[7-(8-ethyl-3-hydroxy-1-naphthyl)-8-fluoro-2-[2-[[4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate

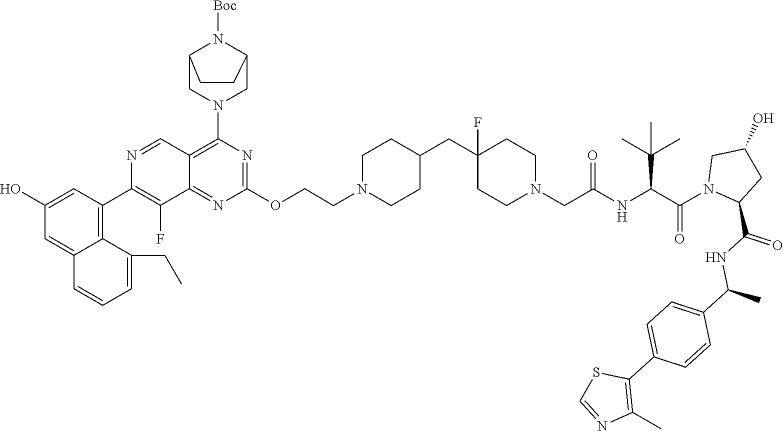 [SMILES: CCc1cccc2cc(O)cc(-c3ncc4c(N5CC6CCC(C5)N6C(=O)OC(C)(C)C)nc(OCCN5CCC(CC6(F)CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12]

To a solution of (2S,4R)-1-[(2S)-2-[2-[[4-fluoro-4-(4-piperidylmethyl)-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (230 mg, 0.3 mmol) and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (197 mg, 0.3 mmol) in dichloromethane (5.0 mL) and isopropyl alcohol (5.0 mL) was added acetic acid (0.08 mL, 1 mmol) and 2-methylpyridine borane (179 mg, 1.7 mmol). The mixture was stirred at 25° C. for 1 h, then concentrated. The crude product was purified by flash column (0-10% methanol in dichloromethane) to give tert-butyl 3-[7-(8-ethyl-3-hydroxy-1-naphthyl)-8-fluoro-2-[2-[[4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate (230 mg, 36%) as a yellow solid. MS (ESI) m/z: 629.1 [M/2+1]⁺.

Step 9: Preparation of (2S,4R)-1-[(2S)-2-[[2-[[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-4-fluoro-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

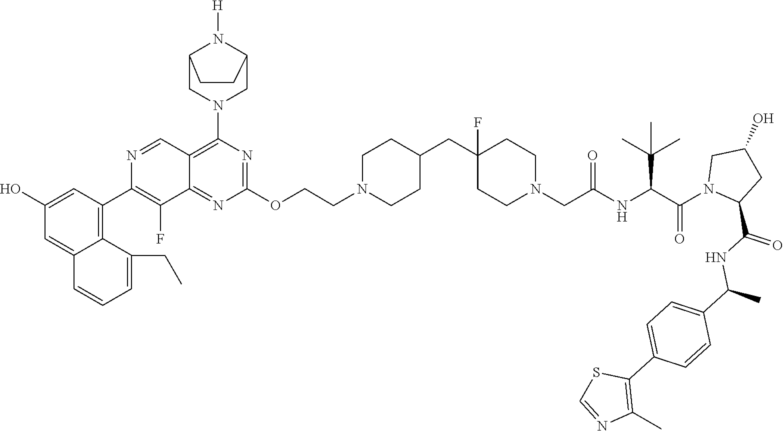 [SMILES: CCc1cccc2cc(O)cc(-c3ncc4c(N5CC6CCC(C5)N6)nc(OCCN5CCC(CC6(F)CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12]

To a solution of tert-butyl 3-[7-(8-ethyl-3-hydroxy-1-naphthyl)-8-fluoro-2-[2-[[4-[[4-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate (330 mg, 0.3 mmol) in CH₂Cl₂ (2.0 mL) was added HCl in dioxane (4 M, 2.0 mL), the reaction mixture was stirred at 25° C. for 0.5 h. The reaction mixture was diluted with petroleum ether (50 mL) and filtered. The filter cake was dissolved in tetrahydrofuran (20 ml), basified with triethylamine to pH 8, filtered and concentrated in vacuum. The crude product was purified by prep-HPLC (column: Xtimate C18 150*40 mm*10 um; mobile phase: [water (NH₄HCO₃)-acetonitrile]; B %: 32%-72%, 40 min) to give (2S,4R)-1-[(2S)-2-[[2-[[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-4-fluoro-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (85 mg, 28%) as a white solid. MS (ESI) m/z: 1157.0 [M+1]⁺; ¹H NMR (400 MHz, CD₃OD) δ 9.04 (s, 1H), 8.88 (s, 1H), 7.63 (d, J=8.4 Hz, 1H), 7.48-7.26 (m, 6H), 7.16 (d, J=6.8 Hz, 1H), 7.02 (d, J=2.8 Hz, 1H), 5.05-4.92 (m, 2H), 4.68-4.52 (m, 7H), 4.44 (s, 1H), 3.84 (s, 1H), 3.78-3.61 (m, 5H), 3.13-3.02 (m, 4H), 2.87 (d, J=5.6 Hz, 2H), 2.74-2.64 (m, 2H), 2.54-2.43 (m, 5H), 2.38-2.18 (m, 5H), 1.99-1.73 (m, 11H), 1.63-1.49 (m, 5H), 1.46-1.23 (m, 3H), 1.06-0.98 (m, 9H), 0.90 (t, J=7.6 Hz, 3H).

Exemplary Synthesis of (2S,4R)-1-[(2S)-2-(2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]-3-fluoroazetidin-1-yl}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide (Compound 263)

Step 1: Preparation of tert-butyl 3-hydroxy-3-(4-pyridylmethyl)azetidine-1-carboxylate

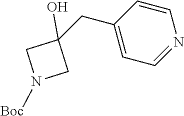 [SMILES: CC(C)(C)OC(=O)N1CC(O)(Cc2ccncc2)C1]

To a mixture of 4-methylpyridine (2.0 g, 2.11 mL, 21 mmol) in THF (20 mL) was added n-BuLi (2.5 M, 10.31 mL) dropwise at −70° C. under N₂. The mixture was stirred at −70° C. for 3 h. Then tert-butyl 3-oxoazetidine-1-carboxylate (3.68 g, 21 mmol) in THF (10 mL) was added dropwise, the solution was stirred at 20° C. for 2 h. The residue was poured into water (20 mL). The aqueous phase was extracted with ethyl acetate (20 mL×3). The combined organic phase was washed with brine (20 mL×2), dried with anhydrous Na₂SO₄, filtered, and concentrated in vacuum. The residue was purified by flash column (0-60% ethyl acetate in petroleum ether) to afford tert-butyl 3-hydroxy-3-(4-pyridylmethyl)azetidine-1-carboxylate (3.31 g, 58%) as a yellow gum. MS (ESI) m/z: 265.2 [M+1]⁺; ¹H NMR (400 MHz, CDCl₃) δ 8.55-8.49 (m, 2H), 7.27-7.24 (m, 2H), 3.97 (d, J=9.6 Hz, 2H), 3.83 (d, J=9.6 Hz, 2H), 3.08 (s, 2H), 1.46-1.44 (m, 9H).

Step 2: Preparation of tert-butyl 3-fluoro-3-(4-pyridylmethyl)azetidine-1-carboxylate

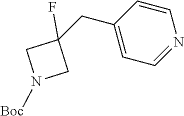 [SMILES: CC(C)(C)OC(=O)N1CC(F)(Cc2ccncc2)C1]

To a mixture of tert-butyl 3-hydroxy-3-(4-pyridylmethyl)azetidine-1-carboxylate (3.31 g, 12 mmol) in CH₂Cl₂ (100 mL) was added DAST (2.42 g, 1.99 mL, 15 mmol) dropwise at −40° C. under N₂. Then the reaction mixture was warmed to 20° C. and stirred for 30 min. The reaction was quenched with aqueous NaHCO₃ to pH 8 at 0° C. The aqueous phase was extracted with CH₂Cl₂ (10 mL×3). The combined organic layers were washed with brine (10 mL×2), dried over anhydrous sodium sulfate, filtered, and concentrated under reduced pressure. The residue was purified by flash column (0-18% ethyl acetate in petroleum ether) to afford tert-butyl 3-fluoro-3-(4-pyridylmethyl)azetidine-1-carboxylate (2.33 g, 70%) as a light-yellow solid. ¹H NMR (400 MHz, CDCl₃) δ 8.56-8.55 (m, 2H), 7.19 (d, J=5.0 Hz, 2H), 4.04-3.90 (m, 4H), 3.27-3.11 (m, 2H), 1.44 (s, 9H).

Step 3: Preparation of benzyl 4-[(1-tert-butoxycarbonyl-3-fluoro-azetidin-3-yl)methyl]piperidine-1-carboxylate

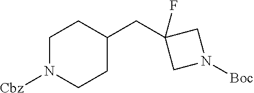 [SMILES: CC(C)(C)OC(=O)N1CC(F)(CC2CCN(C(=O)OCc3ccccc3)CC2)C1]

To a solution of tert-butyl 3-fluoro-3-(4-piperidylmethyl)azetidine-1-carboxylate (1.0 g, 4 mmol) in EtOAc (10 mL) and H₂O (10 mL) were added NaHCO₃ (1.54 g, 18 mmol) and CbzCl (0.52 mL, 3.7 mmol) at 0° C. The mixture was stirred at 25° C. for 10 h. The reaction mixture was diluted with water (20 mL) and extracted with CH₂Cl₂ (50 mL×3). The combined extracts were dried over anhydrous Na₂SO₄, filtered, and concentrated under reduced pressure. The residue was purified by flash column (0-15% THF in petroleum ether) to afford benzyl 4-[(1-tert-butoxycarbonyl-3-fluoro-azetidin-3-yl)methyl]piperidine-1-carboxylate (1.3 g, 87%) as a colorless oil. MS (ESI) m/z: 307.1 [M−100+H]⁺; ¹HNMR (400 MHz, CDCl₃) δ 7.41-7.31 (m, 5H), 5.13 (s, 2H), 4.17 (d, J=7.2 Hz, 2H), 4.12-4.02 (m, 2H), 3.90 (dd, J=10.0, 19.2 Hz, 2H), 2.78 (t, J=12.4 Hz, 2H), 1.87-1.84 (m, 1H), 1.82-1.78 (m, 1H), 1.70-1.65 (m, 3H), 1.46-1.43 (m, 9H), 1.29-1.16 (m, 2H).

Step 4: Preparation of benzyl 4-[(3-fluoroazetidin-3-yl)methyl]piperidine-1-carboxylate

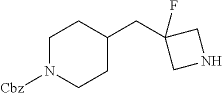 [SMILES: O=C(OCc1ccccc1)N1CCC(CC2(F)CNC2)CC1]

A solution of benzyl 4-[(1-tert-butoxycarbonyl-3-fluoro-azetidin-3-yl)methyl]piperidine-1-carboxylate (800 mg, 2 mmol) in CH₂Cl₂ (6 mL) and 4 M HCl/dioxane (3 mL) was stirred at 25° C. for 20 min. The mixture was concentrated. The residue was dissolved with H₂O (10 mL) and basified with saturated NaHCO₃ solution until pH 8, then the suspension was extracted with CH₂Cl₂/MeOH (40 mL×7, V/V=10/1). The combined extracts was dried over anhydrous Na₂SO₄, filtered, and concentrated under reduced pressure to afford benzyl 4-[(3-fluoroazetidin-3-yl)methyl]piperidine-1-carboxylate (602 mg, crude) as a yellow oil, which was used directly in the next step. MS (ESI) m/z: 307.1 [M+H]⁺.

Step 5: Preparation of benzyl 4-[[1-(2-ethoxy-2-oxo-ethyl)-3-fluoro-azetidin-3-yl]methyl]piperidine-1-carboxylate

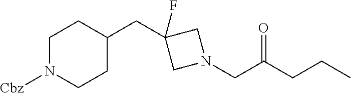 [SMILES: CCCC(=O)CN1CC(F)(CC2CCN(C(=O)OCc3ccccc3)CC2)C1]

To a solution of benzyl 4-[(3-fluoroazetidin-3-yl)methyl]piperidine-1-carboxylate (602 mg, 2 mmol) in THF (10 mL) were added DIEA (0.68 mL, 4 mmol) and ethyl 2-bromoacetate (0.22 mL, 2 mmol). The mixture was stirred at 25° C. for 10 h. The reaction mixture was diluted with water (20 mL) and extracted with CH₂Cl₂ (30 mL×3). The combined extracts were dried over anhydrous Na₂SO₄, filtered, and concentrated under reduced pressure. The residue was purified by flash column (0-40% THF in petroleum ether) to afford benzyl 4-[[1-(2-ethoxy-2-oxo-ethyl)-3-fluoro-azetidin-3-yl]methyl]piperidine-1-carboxylate (550 mg, 71%) as a yellow oil. MS (ESI) m/z: 393.2 [M+H]⁺; ¹H NMR (400 MHz, CDCl₃) δ 7.32-7.22 (m, 5H), 5.05 (s, 2H), 4.11 (q, J=7.2 Hz, 4H), 3.59 (dd, J=9.2, 15.2 Hz, 2H), 3.42-3.24 (m, 4H), 2.70 (s, 2H), 1.88-1.72 (m, 4H), 1.69-1.64 (m, 3H), 1.36 (s, 2H), 1.24-1.19 (m, 3H).

Step 6: Preparation of 2-[3-[(1-benzyloxycarbonyl-4-piperidyl)methyl]-3-fluoro-azetidin-1-yl]acetic acid

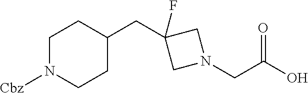 [SMILES: O=C(O)CN1CC(F)(CC2CCN(C(=O)OCc3ccccc3)CC2)C1]

To a solution of benzyl 4-[[1-(2-ethoxy-2-oxo-ethyl)-3-fluoro-azetidin-3-yl]methyl]piperidine-1-carboxylate (676 mg, 1.7 mmol) in THF (12 mL) and H₂O (3 mL) was added LiOH·H₂O (361.4 mg, 8.6 mmol). The reaction mixture was stirred at 25° C. for 10 h, then concentrated and diluted with water (15 mL). The pH of the mixture was adjusted to 4 with 2 N HCl solution, and then lyophilized to afford 2-[3-[(1-benzyloxycarbonyl-4-piperidyl)methyl]-3-fluoro-azetidin-1-yl]acetic acid (627 mg) as a yellow solid, which was used directly in the next step. MS (ESI) m/z: 365.4 [M+H]⁺.

Step 7: Preparation of benzyl 4-[[3-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]piperidine-1-carboxylate

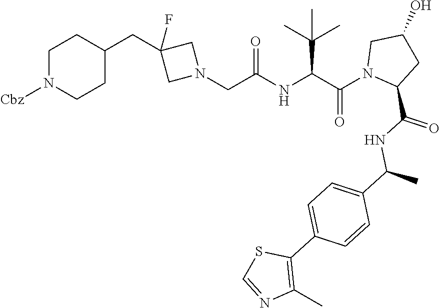 [SMILES: Cc1ncsc1-c1ccc([C@H](C)NC(=O)[C@@H]2C[C@@H](O)CN2C(=O)[C@@H](NC(=O)CN2CC(F)(CC3CCN(C(=O)OCc4ccccc4)CC3)C2)C(C)(C)C)cc1]

To a solution of 2-[3-[(1-benzyloxycarbonyl-4-piperidyl)methyl]-3-fluoro-azetidin-1-yl]acetic acid (627 mg, 1.7 mmol) in DMF (15 mL) were added DIEA (3.0 mL, 17 mmol), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide hydrochloride (827 mg, 1.7 mmol) and HATU (720 mg, 1.9 mmol). The mixture was stirred at 25° C. for 1 h. The reaction mixture was diluted with water (30 mL) and extracted with ethyl acetate (50 mL×3). The combined extracts were washed with brine (70 mL×3), dried over anhydrous Na₂SO₄, filtered, and concentrated under reduced pressure. The residue was purified by flash column (0-7% MeOH in CH₂Cl₂) to afford benzyl 4-[[3-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]piperidine-1-carboxylate (680 mg, 50%) as a yellow solid. MS (ESI) m/z: 791.1 [M+H]⁺.

Step 8: Preparation of (2S,4R)-1-[(2S)-2-[[2-[3-fluoro-3-(4-piperidylmethyl) azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

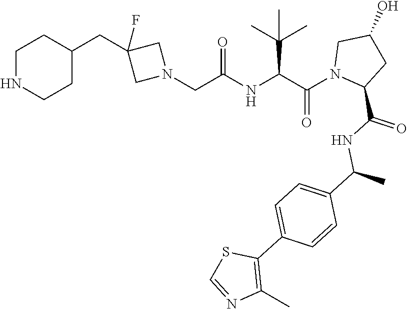 [SMILES: Cc1ncsc1-c1ccc([C@H](C)NC(=O)[C@@H]2C[C@@H](O)CN2C(=O)[C@@H](NC(=O)CN2CC(F)(CC3CCNCC3)C2)C(C)(C)C)cc1]

To a solution of benzyl 4-[[3-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]piperidine-1-carboxylate (400 mg, 0.5 mmol) in TfOH (40 uL) and CH₂Cl₂ (1.2 mL) was added TFA (0.80 mL, 11 mmol). The reaction mixture was stirred at 25° C. for 2 h. The mixture was diluted with saturated Na₂CO₃ solution (5 mL) and CH₂Cl₂ (3 mL), stirred for 30 min. The mixture was diluted with water (30 mL) and extracted with CH₂Cl₂ (50 mL×3). The combined extracts were dried over anhydrous Na₂SO₄, filtered and concentrated under reduced pressure to afford (2S,4R)-1-[(2S)-2-[[2-[3-fluoro-3-(4-piperidylmethyl) azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (332 mg) as a yellow solid, which was used directly in the next step. MS (ESI) m/z: 657.4 [M+H]⁺.

Step 9: Preparation of tert-butyl 3-[7-(8-ethyl-3-hydroxy-1-naphthyl)-8-fluoro-2-[2-[[4-[[3-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]-1-piperidyl]ethoxy]pyrido[4,3-d]pyrimidin-4-yl]-3,8-diazabicyclo[3.2.1]octane-8-carboxylate

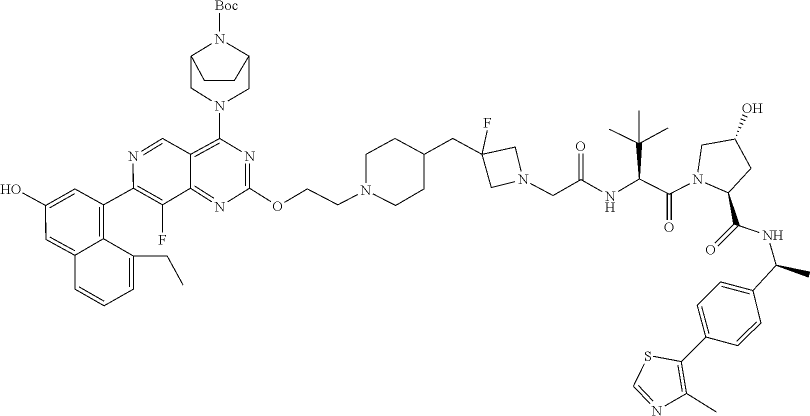 [SMILES: CCc1cccc2cc(O)cc(-c3ncc4c(N5CC6CCC(C5)N6C(=O)OC(C)(C)C)nc(OCCN5CCC(CC6(F)CN(CC(=O)N[C@H](C(=O)N7C[C@H](O)C[C@H]7C(=O)N[C@@H](C)c7ccc(-c8scnc8C)cc7)C(C)(C)C)C6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (250 mg, 0.4 mmol) and (2S,4R)-1-[(2S)-2-[[2-[3-fluoro-3-(4-piperidylmethyl) azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (279 mg, 0.4 mmol) in CH₂Cl₂ (5 mL) and i-PrOH (5 mL) were added AcOH (0.12 mL, 2 mmol) and 2-methylpyridine borane (228 mg, 2 mmol). The mixture was stirred at 25° C. for 1 h, adjusted the pH to 8 with triethylamine and concentrated. The residue was purified by flash column (0-8% methanol in dichloromethane) to afford tert-butyl 3-[7-(8-ethyl-3-hydroxy-1-naphthyl)-8-fluoro-2-[2-[[4-[3-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]-1-piperidyl]ethoxy]pyrido[4,3-d]pyrimidin-4-yl]-3,8-diazabicyclo[3.2.1]octane-8-carboxylate (460 mg, 71%) as a yellow solid. MS (ESI) m/z: 1228.4 [M+H]⁺.

Step 10: Preparation of (2S,4R)-1-[(2S)-2-[[2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-3-fluoro-azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

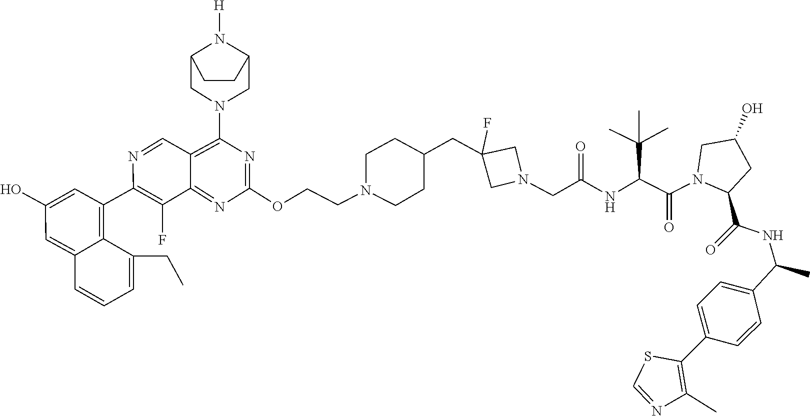 [SMILES: CCc1cccc2cc(O)cc(-c3ncc4c(N5CC6CCC(C5)N6)nc(OCCN5CCC(CC6(F)CN(CC(=O)N[C@H](C(=O)N7C[C@H](O)C[C@H]7C(=O)N[C@@H](C)c7ccc(-c8scnc8C)cc7)C(C)(C)C)C6)CC5)nc4c3F)c12]

A solution of tert-butyl 3-[7-(8-ethyl-3-hydroxy-1-naphthyl)-8-fluoro-2-[2-[[4-[3-fluoro-1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]-1-piperidyl]ethoxy]pyrido[4,3-d]pyrimidin-4-yl]-3,8-diazabicyclo[3.2.1]octane-8-carboxylate (460 mg, 0.3 mmol) in CH₂Cl₂ (6 mL) and TFA (3 mL) was stirred at 25° C. for 30 min. The mixture was basified with saturated NaHCO₃ solution until pH 8. The suspension was extracted with CH₂Cl₂:MeOH (20 mL×5, V:V=10:1). The combined extracts were dried over anhydrous Na₂SO₄, filtered and concentrated under reduced pressure. The residue was purified by prep-HPLC (column: Xtimate C18 150*40 mm*10 um; mobile phase: [water (formic acid)-acetonitrile]; B %: 0%-30%, Gradient time: 36 min) to afford (2S,4R)-1-[(2S)-2-[[2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-3-fluoro-azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (254.0 mg, formic acid salt, 56%) as a white solid. MS (ESI) m/z: 1128.2 [M+H]⁺; ¹H NMR (400 MHz, CD₃OD) δ 9.08 (s, 1H), 8.90-8.81 (m, 1H), 8.49 (s, 1H), 7.63 (d, J=8.4 Hz, 1H), 7.45-7.33 (m, 5H), 7.29 (d, J=2.4 Hz, 1H), 7.16 (d, J=7.2 Hz, 1H), 7.01 (d, J=2.4 Hz, 1H), 5.02-4.98 (m, 1H), 4.79-4.41 (m, 10H), 3.89-3.67 (m, 6H), 3.58-3.36 (m, 7H), 2.76-2.60 (m, 2H), 2.47 (s, 3H), 2.39-2.17 (m, 3H), 2.02-1.81 (m, 10H), 1.58-1.46 (m, 5H), 1.06-1.00 (m, 9H), 0.90 (t, J=7.2 Hz, 3H).

Exemplary Synthesis of (2S,4R)-1-[(2R*)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide (Compound 265)

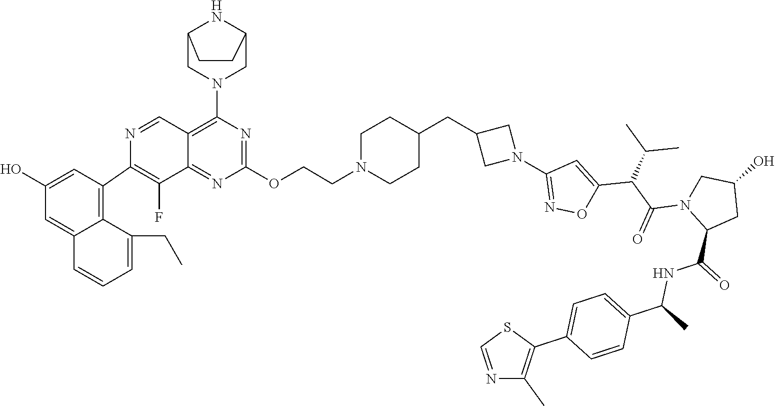

The title compound was prepared analogously to (2S,4R)-1-[(2R*)-2-(3-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}-1,2-oxazol-5-yl)-3-methylbutanoyl]-4-hydroxy-N-[(1S)-1-[4-(1-methyl-1H-pyrazol-5-yl)phenyl]ethyl]pyrrolidine-2-carboxamide, starting from (2S,4R)-4-hydroxy-1-[(2S)-3-methyl-2-[3-[[3-(4-piperidylmethyl) azetidin-1-yl]isoxazol-5-yl]butanoyl]-N-[(1S)-1-[4-(2-methylpyrazol-3-yl)phenyl]ethyl]pyrrolidine-2-carboxamide and tert-butyl 3-[7-[8-ethyl-3-(methoxymethoxy)-1-naphthyl]-8-fluoro-2-(2-oxoethoxy)pyrido[4,3-d]pyrimidin-4-yl]-3,8-diazabicyclo[3.2.1]octane-8-carboxylate. (off-white solid). MS (ESI) m/z: 1089.8 [M+1]⁺; 1H NMR (400 MHz, MeOD-d₄) δ 9.04 (s, 1H), 7.62 (d, J=8.0 Hz, 1H), 7.52-7.31 (m, 6H), 7.28 (d, J=2.4 Hz, 1H), 7.16 (d, J=7.2 Hz, 1H), 7.01 (d, J=2.4 Hz, 1H), 6.38-6.27 (m, 1H), 5.89-5.79 (m, 1H), 5.03-4.95 (m, 1H), 4.67-4.52 (m, 7H), 4.47-4.36 (m, 1H), 4.09-3.93 (m, 2H), 3.88-3.81 (m, 3H), 3.77-3.62 (m, 6H), 3.58-3.45 (m, 2H), 3.07 (br d, J=10.4 Hz, 2H), 2.91-2.76 (m, 3H), 2.44-2.10 (m, 6H), 2.05-1.74 (m, 5H), 1.72-1.61 (m, 2H), 1.57 (br t, J=6.8 Hz, 2H), 1.49 (d, J=7.2 Hz, 2H), 1.36-1.20 (m, 3H), 1.06-0.93 (m, 3H), 0.92-0.80 (m, 6H).

Exemplary Synthesis of (2S,4R)-1-[(2S)-2-(2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]azetidin-1-yl}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide (Compound 273)

Step 1: Preparation of tert-butyl 4-[[1-(2-ethoxy-2-oxo-ethyl) azetidin-3-yl]methyl]piperidine-1-carboxylate

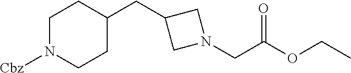 [SMILES: CCOC(=O)CN1CC(CC2CCN(C(=O)OCc3ccccc3)CC2)C1]

To a solution of tert-butyl 4-(azetidin-3-ylmethyl)piperidine-1-carboxylate (382 mg, 1.5 mmol) in DMF (5 mL) were added K₂CO₃ (415.0 mg, 3 mmol) and ethyl 2-bromoacetate (0.15 mL, 1.3 mmol). The mixture was stirred at 25° C. for 10 h. Then diluted with water (50 mL) and extracted with ethyl acetate (50 mL×3). The combined extracts were washed with brine (20 mL×3), dried over anhydrous Na₂SO₄, filtered, and concentrated under reduced pressure. The residue was purified by flash column (0-25% THF in petroleum ether) to afford tert-butyl 4-[[1-(2-ethoxy-2-oxo-ethyl) azetidin-3-yl]methyl]piperidine-1-carboxylate (380 mg, 74%) as a yellow solid. MS (ESI) m/z: 341.2 [M+H]⁺.

Step 2: Preparation of 2-[3-[(1-tert-butoxycarbonyl-4-piperidyl)methyl]azetidin-1-yl]acetic acid

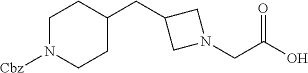 [SMILES: O=C(O)CN1CC(CC2CCN(C(=O)OCc3ccccc3)CC2)C1]

To a solution of tert-butyl 4-[[1-(2-ethoxy-2-oxo-ethyl) azetidin-3-yl]methyl]piperidine-1-carboxylate (380 mg, 1 mmol) in THF (20 mL) and H₂O (5 mL) was added LiOH·H₂O (234 mg, 5.6 mmol). The reaction mixture was stirred at 25° C. for 10 h, then concentrated and diluted with water (20 mL). The pH of the mixture was adjusted to 4 with 2N HCl, and then lyophilized to afford 2-[3-[(1-tert-butoxycarbonyl-4-piperidyl)methyl]azetidin-1-yl]acetic acid (348 mg, crude) as a white solid, which was used directly in the next step. MS (ESI) m/z: 313.2 [M+H]⁺.

Step 3: Preparation of tert-butyl 4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]piperidine-1-carboxylate

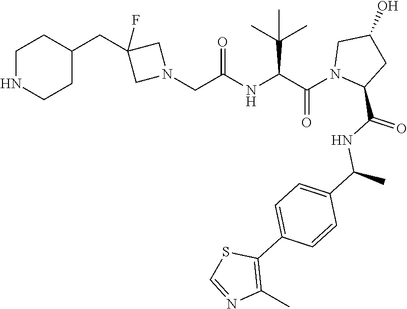 [SMILES: Cc1ncsc1-c1ccc([C@H](C)NC(=O)[C@@H]2C[C@@H](O)CN2C(=O)[C@@H](NC(=O)CN2CC(F)(CC3CCNCC3)C2)C(C)(C)C)cc1]

To a solution of 2-[3-[(1-tert-butoxycarbonyl-4-piperidyl)methyl]azetidin-1-yl]acetic acid (348 mg, 1 mmol) in CH₂Cl₂ (10 mL) were added DIEA (1.94 mL, 11 mmol), (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide hydrochloride (589 mg, 1.23 mmol) and HATU (466 mg, 1.23 mmol). The mixture was stirred at 25° C. for 3 h, then diluted with water (50 mL) and extracted with CH₂Cl₂ (70 mL×3). The combined extracts were washed with brine (100 mL×2), dried over anhydrous Na₂SO₄, filtered, and concentrated under reduced pressure. The residue was purified by flash column (0-10% MeOH in CH₂Cl₂) to afford tert-butyl 4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]piperidine-1-carboxylate (187 mg, 23%) as a yellow solid. MS (ESI) m/z: 739.4 [M+H]⁺.

Step 4: Preparation of (2S,4R)-1-[(2S)-3,3-dimethyl-2-[[2-[3-(4-piperidylmethyl) azetidin-1-yl]acetyl]amino]butanoyl]-4-hydroxy-N-[(1S)-1-[4-(4-methylthiazol-5-yl)phenyl]ethyl]pyrrolidine-2-carboxamide

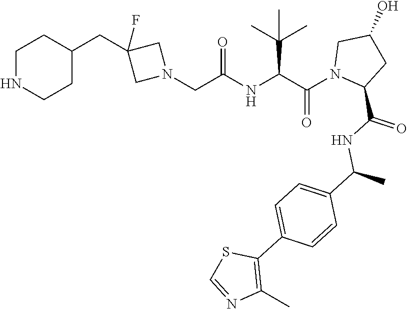 [SMILES: Cc1ncsc1-c1ccc([C@H](C)NC(=O)[C@@H]2C[C@@H](O)CN2C(=O)[C@@H](NC(=O)CN2CC(F)(CC3CCNCC3)C2)C(C)(C)C)cc1]

A solution of tert-butyl 4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]piperidine-1-carboxylate (187 mg, 0.2 mmol) in CH₂Cl₂ (6 mL) and TFA (2 mL) was stirred at 25° C. for 30 min. The reaction was concentrated, the residue was basified with saturated NaHCO₃ solution until pH 8, the suspension was extracted with CH₂Cl₂ (20 mL×3). The combined extracts were washed with brine (15 mL×3), dried over anhydrous Na₂SO₄, filtered and concentrated under reduced pressure to afford (2S,4R)-1-[(2S)-3,3-dimethyl-2-[[2-[3-(4-piperidylmethyl) azetidin-1-yl]acetyl]amino]butanoyl]-4-hydroxy-N-[(1S)-1-[4-(4-methylthiazol-5-yl)phenyl]ethyl]pyrrolidine-2-carboxamide (161 mg, crude) as a yellow oil, which was used directly in the next step. MS (ESI) m/z: 639.1 [M+H]⁺.

Step 5: Preparation of tert-butyl 3-[7-(8-ethyl-3-hydroxy-1-naphthyl)-8-fluoro-2-[2-[[4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]-1-piperidyl]ethoxy]pyrido[4,3-d]pyrimidin-4-yl]-3,8-diazabicyclo[3.2.1]octane-8-carboxylate

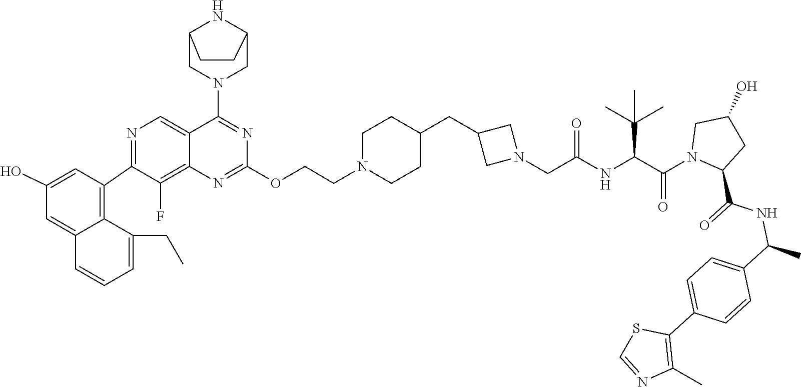 [SMILES: CCc1cccc2cc(O)cc(-c3ncc4c(N5CC6CCC(C5)N6)nc(OCCN5CCC(CC6CN(CC(=O)N[C@H](C(=O)N7C[C@H](O)C[C@H]7C(=O)N[C@@H](C)c7ccc(-c8scnc8C)cc7)C(C)(C)C)C6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (125 mg, 0.2 mmol) and (2S,4R)-1-[(2S)-3,3-dimethyl-2-[[2-[3-(4-piperidylmethyl) azetidin-1-yl]acetyl]amino]butanoyl]-4-hydroxy-N-[(1S)-1-[4-(4-methylthiazol-5-yl)phenyl]ethyl]pyrrolidine-2-carboxamide (157.0 mg, 0.2 mmol) in CH₂Cl₂ (5 mL) and i-PrOH (5 mL) were added AcOH (61 uL, 1 mmol) and 2-methylpyridine borane (114.0 mg, 1 mmol). The mixture was stirred at 25° C. for 1 h, adjusted the pH to 8 with triethylamine and concentrated under reduced pressure. The residue was purified by flash column (0-7% methanol in dichloromethane) to afford tert-butyl 3-[7-(8-ethyl-3-hydroxy-1-naphthyl)-8-fluoro-2-[2-[[4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]-1-piperidyl]ethoxy]pyrido[4,3-d]pyrimidin-4-yl]-3,8-diazabicyclo[3.2.1]octane-8-carboxylate (185 mg, 63%) as a yellow solid. MS (ESI) m/z: 1210.4 [M+H]⁺.

Step 6: Preparation of (2S,4R)-1-[(2S)-2-[[2-[3-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

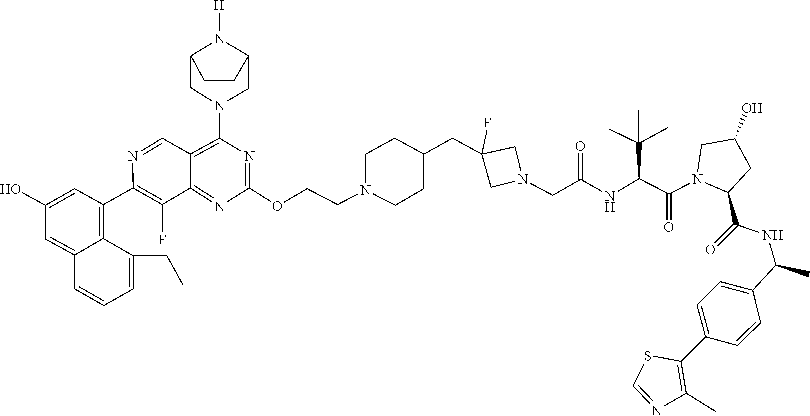 [SMILES: CCc1cccc2cc(O)cc(-c3ncc4c(N5CC6CCC(C5)N6)nc(OCCN5CCC(CC6(F)CN(CC(=O)N[C@H](C(=O)N7C[C@H](O)C[C@H]7C(=O)N[C@@H](C)c7ccc(-c8scnc8C)cc7)C(C)(C)C)C6)CC5)nc4c3F)c12]

A solution of tert-butyl 3-[7-(8-ethyl-3-hydroxy-1-naphthyl)-8-fluoro-2-[2-[[4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]azetidin-3-yl]methyl]-1-piperidyl]ethoxy]pyrido[4,3-d]pyrimidin-4-yl]-3,8-diazabicyclo[3.2.1]octane-8-carboxylate (185 mg, 0.1 mmol) in CH₂Cl₂ (6 mL) and TFA (3 mL) was stirred at 25° C. for 30 min. The mixture was dried under N₂, the residue was basified with saturated NaHCO₃ solution until pH 8, extracted with CH₂Cl₂:MeOH (20 mL×5, V:V=10:1). The combined extracts were dried over anhydrous Na₂SO₄, filtered, and concentrated under reduced pressure. The residue was purified by prep-HPLC (column: Xtimate C18 150*40 mm*10 um; mobile phase: [water (formic acid)-acetonitrile]; B %: 0%-30%, Gradient time: 36 min) to afford (2S,4R)-1-[(2S)-2-[[2-[3-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]azetidin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (80.1 mg, formic acid salt, 45%) as a white solid. MS (ESI) m/z: 1110.8 [M+1]⁺; ¹H NMR (400 MHz, MeOD-d₄) δ 9.13-9.08 (m, 1H), 8.90-8.82 (m, 1H), 8.52-8.43 (m, 2H), 7.64 (d, J=8.0 Hz, 1H), 7.47-7.34 (m, 5H), 7.30 (d, J=2.4 Hz, 1H), 7.17 (d, J=6.8 Hz, 1H), 7.01 (d, J=2.4 Hz, 1H), 5.00 (d, J=7.2 Hz, 1H), 4.81-4.70 (m, 6H), 4.63-4.53 (m, 2H), 4.46-4.38 (m, 1H), 4.06-3.99 (m, 2H), 3.95-3.83 (m, 4H), 3.77-3.68 (m, 1H), 3.63-3.49 (m, 4H), 3.37 (s, 3H), 2.88-2.69 (m, 3H), 2.53-2.44 (m, 3H), 2.41-2.18 (m, 3H), 2.07-1.80 (m, 7H), 1.68-1.41 (m, 8H), 1.09-0.99 (m, 9H), 0.94-0.85 (m, 3H).

Exemplary Synthesis of (2S,4R)-1-[(2S)-2-(2-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]piperidin-1-yl}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide (Compound 274)

Step 1: Preparation of tert-butyl 3-[7-(8-ethyl-3-hydroxy-1-naphthyl)-8-fluoro-2-[2-[[4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate

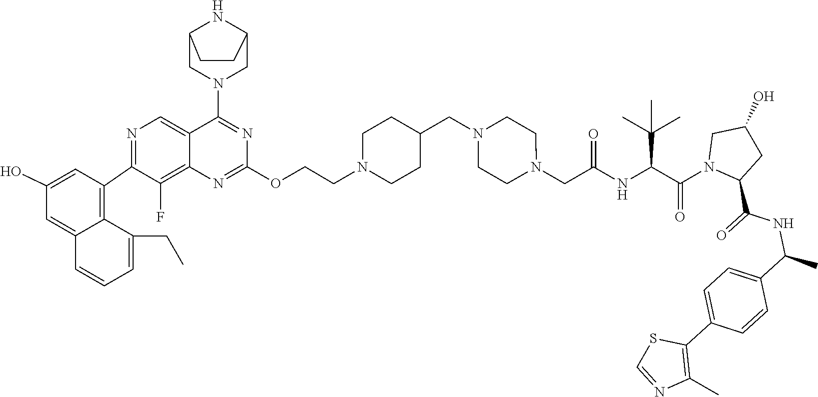 [SMILES: CCc1cccc2cc(O)cc(-c3ncc4c(N5CC6CCC(C5)N6)nc(OCCN5CCC(CN6CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12]

To a mixture of (2S,4R)-1-[(2S)-3,3-dimethyl-2-[2-[[4-(4-piperidylmethyl)-1-piperidyl]acetyl]amino]butanoyl]-4-hydroxy-N-[(1S)-1-[4-(4-methylthiazol-5-yl)phenyl]ethyl]pyrrolidine-2-carboxamide (230 mg, 0.3 mmol) and tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (223 mg, 0.4 mmol) in dichloromethane (5 mL) and i-PrOH (5 mL) were added AcOH (79 uL, 1.4 mmol) and 2-methylpyridine borane (184 mg, 1.7 mmol). The mixture was stirred at 25° C. for 1 h, then concentrated in vacuum. The pH of the residue was adjusted to 10 by triethylamine. The residue was purified by column chromatography (0˜5% then up to 10% methanol in dichloromethane) to afford tert-butyl 3-[7-(8-ethyl-3-hydroxy-1-naphthyl)-8-fluoro-2-[2-[[4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate (258 mg, 38%) as a yellow solid. MS (ESI) m/z: 1238.5 [M+H]⁺.

Step 2: Preparation of (2S,4R)-1-[(2S)-2-[[2-[[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

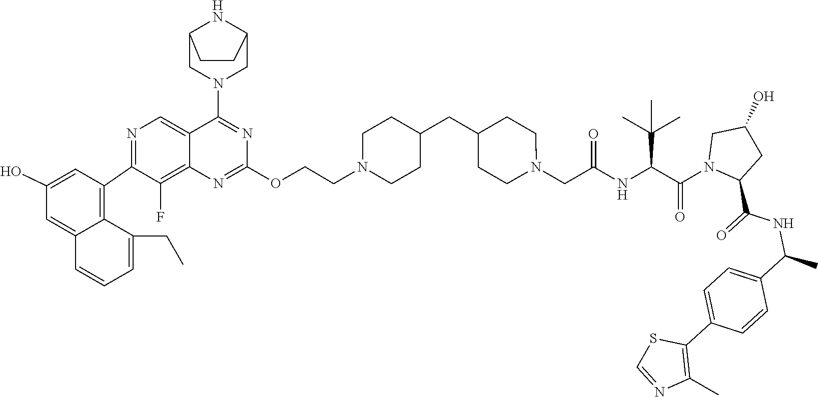 [SMILES: CCc1cccc2cc(O)cc(-c3ncc4c(N5CC6CCC(C5)N6)nc(OCCN5CCC(CC6CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12]

To a mixture of tert-butyl 3-[7-(8-ethyl-3-hydroxy-1-naphthyl)-8-fluoro-2-[2-[[4-[[1-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]-4-piperidyl]methyl]-1-piperidyl]ethoxy]pyrido[4,3-d]pyrimidin-4-yl]-3,8-diazabicyclo[3.2.1]octane-8-carboxylate (253 mg, 0.1 mmol) in dichloromethane (4 mL) was added HCl in dioxane (4 M, 2 mL). The mixture was stirred at 25° C. for 30 min, then was concentrated in vacuum. The residue was re-dissolved in tetrahydrofuran and adjusted the pH to 10 by triethylamine, then filtered and concentrated. The residue was purified by prep-HPLC (column: Xtimate C18 150*40 mm*10 um; mobile phase: [water (formic acid)-acetonitrile]; B %: 0%-38%, 36 min) to afford (2S,4R)-1-[(2S)-2-[2-[[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]-1-piperidyl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (56.6 mg, formic acid salt, 22%) as a white solid. MS (ESI) m/z: 1139.0 [M+H]⁺; ¹H NMR (400 MHz, CD₃OD) δ 9.11 (s, 1H), 8.88 (s, 1H), 8.47 (s, 2H), 7.64 (d, J=8.0 Hz, 1H), 7.46-7.35 (m, 5H), 7.31 (d, J=2.4 Hz, 1H), 7.21-7.14 (m, 1H), 7.01 (d, J=2.4 Hz, 1H), 5.07-5.02 (m, 1H), 4.65-4.36 (m, 9H), 3.98 (s, 2H), 3.92-3.80 (m, 3H), 3.75 (dd, J=3.2, 10.9 Hz, 1H), 3.63-3.54 (m, 2H), 3.45 (s, 2H), 3.19-3.09 (m, 2H), 3.00-2.86 (m, 4H), 2.48 (s, 3H), 2.38-2.20 (m, 5H), 2.04-1.91 (m, 6H), 1.78-1.66 (m, 3H), 1.51 (d, J=6.8. Hz, 3H), 1.43 (d, J=12.4 Hz, 2H), 1.39-1.29 (m, 2H), 1.28-1.22 (m, 2H), 1.07-1.01 (m, 9H), 0.90 (t, J=7.2 Hz, 3H).

Exemplary Synthesis of (2S,4R)-1-[(2S)-2-(2-{4-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methyl]piperazin-1-yl}acetamido)-3,3-dimethylbutanoyl]-4-hydroxy-N-[(1S)-1-[4-(4-methyl-1,3-thiazol-5-yl)phenyl]ethyl]pyrrolidine-2-carboxamide (Compound 281)

Step 1: Preparation of (2S,4R)-1-[(2S)-2-[[2-[[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

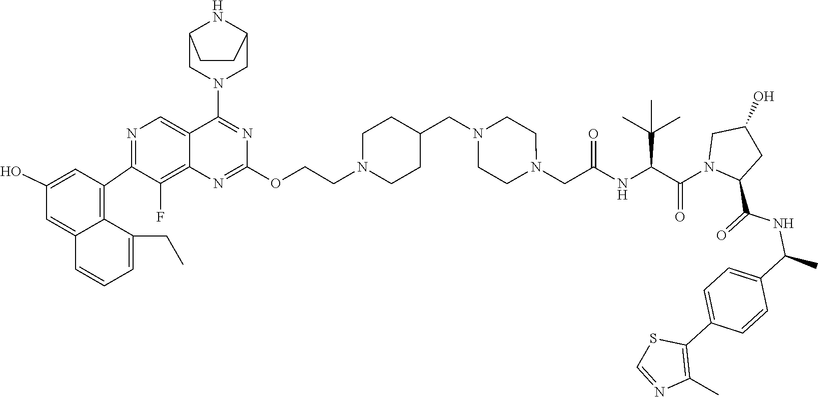 [SMILES: CCc1cccc2cc(O)cc(-c3ncc4c(N5CC6CCC(C5)N6)nc(OCCN5CCC(CN6CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (127 mg, 0.2 mmol), (2S,4R)-1-[(2S)-3,3-dimethyl-2-[[2-[[4-(4-piperidylmethyl) piperazin-1-yl]acetyl]amino]butanoyl]-4-hydroxy-N-[(1S)-1-[4-(4-methylthiazol-5-yl)phenyl]ethyl]pyrrolidine-2-carboxamide (120 mg, 0.2 mmol) in CH₂Cl₂ (10 mL) and i-PrOH (2 mL) was added acetic acid (51 uL, 0.9 mmol) and 2-methylpyridine borane (96 mg, 0.9 mmol). The mixture was stirred at 25° C. for 1 h, then concentrated in vacuum. The residue was purified by flash column (0-10% methanol in dichloromethane) to afford tert-butyl 3-[7-(8-ethyl-3-hydroxy-1-naphthyl)-8-fluoro-2-[2-[[4-[[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]piperazin-1-yl]methyl]-1-piperidyl]ethoxy]pyrido[4,3-d]pyrimidin-4-yl]-3,8-diazabicyclo[3.2.1]octane-8-carboxylate (130 mg, 52% yield) as a white solid. MS (ESI) m/z: 1239.5 [M+1]⁺.

Step 2: Preparation of (2S,4R)-1-[(2S)-2-[[2-[[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide

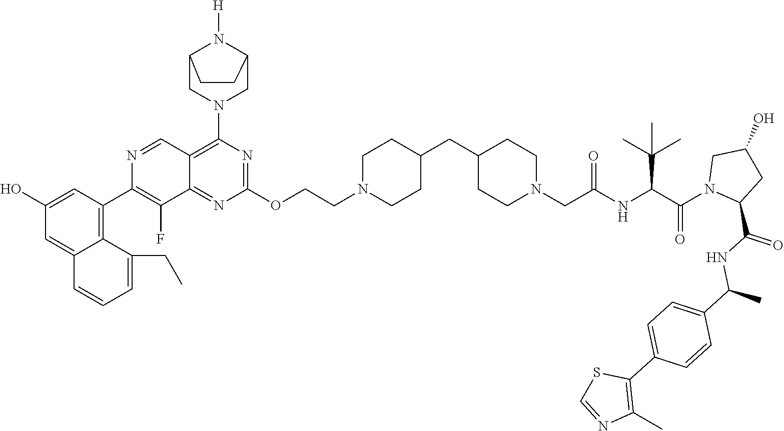 [SMILES: CCc1cccc2cc(O)cc(-c3ncc4c(N5CC6CCC(C5)N6)nc(OCCN5CCC(CC6CCN(CC(=O)N[C@H](C(=O)N7C[C@H](O)C[C@H]7C(=O)N[C@@H](C)c7ccc(-c8scnc8C)cc7)C(C)(C)C)CC6)CC5)nc4c3F)c12]

To a solution of tert-butyl 3-[7-(8-ethyl-3-hydroxy-1-naphthyl)-8-fluoro-2-[2-[[4-[[4-[2-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-2-oxo-ethyl]piperazin-1-yl]methyl]-1-piperidyl]ethoxy]pyrido[4,3-d]pyrimidin-4-yl]-3,8-diazabicyclo[3.2.1]octane-8-carboxylate (130 mg, 0.1 mmol) in CH₂Cl₂ (2.0 mL) was added HCl in dioxane (1.0 mL, 4 M). The mixture was stirred at 25° C. for 30 min. The mixture was diluted with petroleum ether (50 mL) and filtered. The filter cake was dissolved in tetrahydrofuran (30 mL), added triethylamine (0.2 mL), filtered and concentrated in vacuum. The residue was purified by prep-HPLC (column: Welch Xtimate C18 150*40 mm*10 um; mobile phase: [water (formic acid)-acetonitrile]; B %: 0%-28%, 25 min) to afford (2S,4R)-1-[(2S)-2-[2-[[4-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methyl]piperazin-1-yl]acetyl]amino]-3,3-dimethyl-butanoyl]-4-hydroxy-N-[(1S)-1-[4-(4-methylthiazol-5-yl)phenyl]ethyl]pyrrolidine-2-carboxamide (42 mg, 32%, di-formic acid) as a white solid. MS (ESI) m/z: 1140.0 [M+1]⁺; 1H NMR (400 MHz, CD₃OD) δ 9.11 (s, 1H), 8.87 (s, 1H), 8.46 (s, 2H), 7.64 (d, J=8.0 Hz, 1H), 7.46-7.30 (m, 6H), 7.17 (d, J=7.2 Hz, 1H), 7.01 (d, J=2.8 Hz, 1H), 5.01 (q, J=6.8 Hz, 2H), 4.82-4.72 (m, 5H), 4.63 (s, 1H), 4.56 (t, J=8.4 Hz, 1H), 4.48-4.43 (m, 1H), 4.11 (d, J=9.2 Hz, 2H), 3.97-3.83 (m, 3H), 3.77-3.72 (m, 1H), 3.61 (d, J=11.2 Hz, 2H), 3.49-3.41 (m, 2H), 3.13-3.07 (m, 2H), 2.98-2.87 (m, 2H), 2.65 (s, 8H), 2.47 (s, 3H), 2.42-2.19 (m, 5H), 2.10-1.94 (m, 7H), 1.54-1.46 (m, 4H), 1.04 (s, 9H), 0.90 (t, J=7.2 Hz, 3H).

Exemplary Synthesis of (2S,4R)-1-[(2S)-2-{2-[(1-{2-[(4-{3,8-diazabicyclo[3.2.1]octan-3-yl}-7-(8-ethyl-3-hydroxynaphthalen-1-yl)pyrimido[4,5-d][1,3]diazin-2-yl)oxy]ethyl}piperidin-4-yl)methoxy]acetamido}-3,3-dimethylbutanoyl]-N-[(1S)-1-[4-(4-ethyl-1,3-thiazol-5-yl)phenyl]ethyl]-4-hydroxypyrrolidine-2-carboxamide (Compound 295)

Step 1: Preparation of tert-butyl N-[(1S)-1-[4-(4-ethylthiazol-5-yl)phenyl]ethyl]carbamate

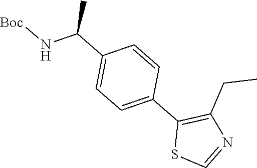 [SMILES: CCc1ncsc1-c1ccc([C@H](C)NC(=O)OC(C)(C)C)cc1]

A mixture of 4-ethylthiazole (1.1 g, 9.7 mmol), tert-butyl N-[(1S)-1-(4-bromophenyl)ethyl]carbamate (1.46 g, 4.9 mmol), potassium carbonate (1.34 g, 9.7 mmol), tricyclohexylphosphonium tetrafluoroborate (89 mg, 0.2 mmol), 2,2-dimethylpropanoic acid (149 mg, 1 mmol, 0.2 mL) and palladium acetate (109 mg, 0.5 mmol) in N,N-dimethyl acetamide (10 mL) was degassed and purged with nitrogen for 3 times. The mixture was stirred at 100° C. for 12 h under nitrogen atmosphere. The residue was poured into water (50 mL) and stirred for 5 min. The aqueous phase was extracted with ethyl acetate (20 mL×3). The combined organic phase was washed with brine (20 mL×3), dried with anhydrous sodium sulfate, filtered and concentrated in vacuum. The residue was purified by flash silica gel chromatography (0-25% ethyl acetate/petroleum ether) to afford tert-butyl N-[(1S)-1-[4-(4-ethylthiazol-5-yl)phenyl]ethyl]carbamate (1.4 g, 86%) as white solid. MS (ESI) m/z: 333.2 [M+1]⁺.

Step 2: Preparation of (1S)-1-[4-(4-ethylthiazol-5-yl)phenyl]ethanamine

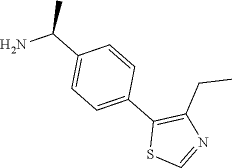 [SMILES: CCc1ncsc1-c1ccc([C@H](C)N)cc1]

To a solution of tert-butyl N-[(1S)-1-[4-(4-ethylthiazol-5-yl)phenyl]ethyl]carbamate (1.4 g, 4 mmol) in dichloromethane (10 mL) was added hydrochloric acid in dioxane (4 M, 5.3 mL). The mixture was stirred at 25° C. for 2 h, then concentrated in vacuum to afford (1S)-1-[4-(4-ethylthiazol-5-yl)phenyl]ethanamine hydrochloride (1.1 g, crude) as a white oil.

Step 3: Preparation of tert-butyl(2S,4R)-2-[[(1S)-1-[4-(4-ethylthiazol-5-yl)phenyl]ethyl]carbamoyl]-4-hydroxy-pyrrolidine-1-carboxylate

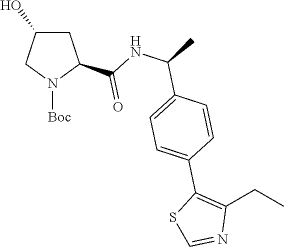 [SMILES: CCc1ncsc1-c1ccc([C@H](C)NC(=O)[C@@H]2C[C@@H](O)CN2C(=O)OC(C)(C)C)cc1]

A mixture of (1S)-1-[4-(4-ethylthiazol-5-yl)phenyl]ethanamine (1.1 g, 4 mmol, hydrochloride), N,N-diisopropylethylamine (1.59 g, 12 mmol, 2.1 mL) in N,N-dimethylformamide (5 mL), was added (2S,4R)-1-tert-butoxycarbonyl-4-hydroxy-pyrrolidine-2-carboxylic acid (851 mg, 3.7 mmol), degassed and purged with nitrogen for 3 times, then O-(7-Azabenzotriazol-1-yl)-N,N,N′,N′-tetramethyluronium Hexafluorophosphate (2.33 g, 6 mmol) was added into the mixture at 0° C. The mixture was stirred at 20° C. for 1 h under nitrogen atmosphere. The residue was poured into ice-water (30 mL) and stirred for 3 min. The aqueous phase was extracted with ethyl acetate (10 mL×3). The combined organic phase was washed with brine (10 mL×3), dried with anhydrous sodium sulfate, filtered, and concentrated in vacuum. The residue was purified by flash silica gel chromatography (0-100% ethyl acetate/petroleum ether) to afford tert-butyl(2S,4R)-2-[[(1S)-1-[4-(4-ethylthiazol-5-yl)phenyl]ethyl]carbamoyl]-4-hydroxy-pyrrolidine-1-carboxylate (620 mg, 33%) as a green oil. MS (ESI) m/z: 446.2 [M+1]⁺.

Step 4: Preparation of (2S,4R)—N-[(1S)-1-[4-(4-ethylthiazol-5-yl)phenyl]ethyl]-4-hydroxy-pyrrolidine-2-carboxamide

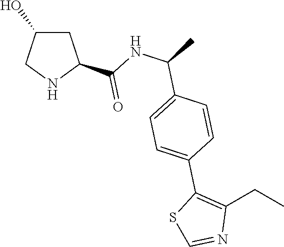 [SMILES: CCc1ncsc1-c1ccc([C@H](C)NC(=O)[C@@H]2C[C@@H](O)CN2)cc1]

To a solution of tert-butyl(2S,4R)-2-[[(1S)-1-[4-(4-ethylthiazol-5-yl)phenyl]ethyl]carbamoyl]-4-hydroxy-pyrrolidine-1-carboxylate (620 mg, 1.4 mmol) in dichloromethane (3 mL) was added hydrochloric in dioxane (4 M, 2 mL). The mixture was stirred at 25° C. for 2 h, then concentrated in vacuum to afford (2S,4R)—N-[(1S)-1-[4-(4-ethylthiazol-5-yl)phenyl]ethyl]-4-hydroxy-pyrrolidine-2-carboxamide hydrochloride (520 mg, crude) as a white solid.

Step 5: Preparation of tert-butyl N-[(1S)-1-[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]carbamate

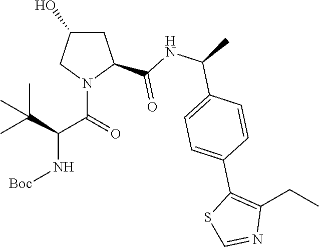 [SMILES: CCc1ncsc1-c1ccc([C@H](C)NC(=O)[C@@H]2C[C@@H](O)CN2C(=O)[C@@H](NC(=O)OC(C)(C)C)C(C)(C)C)cc1]

A mixture of (2S,4R)—N-[(1S)-1-[4-(4-ethylthiazol-5-yl)phenyl]ethyl]-4-hydroxy-pyrrolidine-2-carboxamide hydrochloride (520 mg, 1.4 mmol), N,N-diisopropylethylamine (528 mg, 4 mmol, 0.71 mL) in N,N-dimethyl formamide (3 mL) was added (2S)-2-(tert-butoxycarbonylamino)-3,3-dimethyl-butanoic acid (315 mg, 1.4 mmol), degassed and purged with nitrogen for 3 times, then O-(7-Azabenzotriazol-1-yl)-N,N,N′,N′-tetramethyluronium Hexafluorophosphate (776 mg, 2 mmol) was added to the mixture at 0° C. The mixture was stirred at 20° C. for 1 h under nitrogen atmosphere. The reaction was poured into ice-water (30 mL) and stirred for 3 min. The aqueous phase was extracted with ethyl acetate (10 mL×3). The combined organic phase was washed with brine (10 mL×3), dried with anhydrous sodium sulfate, filtered, and concentrated in vacuum. The residue was purified by prep-HPLC (column: Unisil 3-100 C18 Ultra 150*50 mm*3 um; mobile phase: [water (formic acid)-acetonitrile]; B %: 46%-76%, 7 min) to afford tert-butyl N-[(1S)-1-[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]carbamate (250 mg, 33%) as a white solid. MS (ESI) m/z: 559.3 [M+1]⁺.

Step 6: Preparation of (2S,4R)-1-[(2S)-2-amino-3,3-dimethyl-butanoyl]-N-[(1S)-1-[4-(4-ethylthiazol-5-yl)phenyl]ethyl]-4-hydroxy-pyrrolidine-2-carboxamide

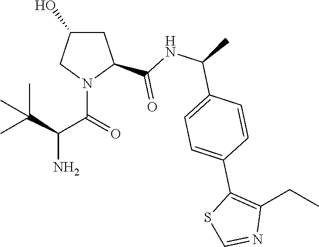 [SMILES: CCc1ncsc1-c1ccc([C@H](C)NC(=O)[C@@H]2C[C@@H](O)CN2C(=O)[C@@H](N)C(C)(C)C)cc1]

To a solution of tert-butyl N-[(1S)-1-[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]carbamate (250 mg, 0.45 mmol) in dichloromethane (10 mL) was added hydrochloric acid in dioxane (4 M, 1 mL). The mixture was stirred at 25° C. for 2 h, then concentrated in vacuum to afford (2S,4R)-1-[(2S)-2-amino-3,3-dimethyl-butanoyl]-N-[(1S)-1-[4-(4-ethylthiazol-5-yl)phenyl]ethyl]-4-hydroxy-pyrrolidine-2-carboxamide (205 mg, crude, hydrochloride salt) as a white solid. MS (ESI) m/z: 459.1 [M+1]⁺.

Step 7: Preparation of tert-butyl 4-[[2-[[(1S)-1-[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]amino]-2-oxo-ethoxy]methyl]piperidine-1-carboxylate

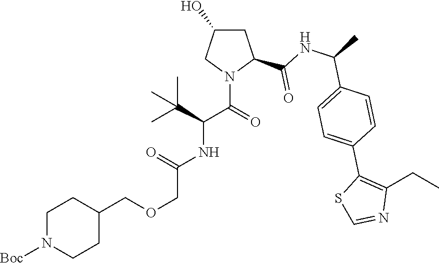 [SMILES: CCc1ncsc1-c1ccc([C@H](C)NC(=O)[C@@H]2C[C@@H](O)CN2C(=O)[C@@H](NC(=O)COCC2CCN(C(=O)OC(C)(C)C)CC2)C(C)(C)C)cc1]

A mixture of (2S,4R)-1-[(2S)-2-amino-3,3-dimethyl-butanoyl]-N-[(1S)-1-[4-(4-ethylthiazol-5-yl)phenyl]ethyl]-4-hydroxy-pyrrolidine-2-carboxamide (205 mg, 0.5 mmol), N,N-diisopropylethylamine (173 mg, 1 mmol, 0.2 mL) in N,N-dimethylformamide (3 mL) was added 2-[(1-tert-butoxycarbonyl-4-piperidyl)methoxy]acetic acid (122 mg, 0.5 mmol), degassed and purged with nitrogen for 3 times, then O-(7-Azabenzotriazol-1-yl)-N,N,N′,N′-tetramethyluronium Hexafluorophosphate (255 mg, 0.7 mmol) was added to the mixture at 0° C. The mixture was stirred at 20° C. for 1 h under nitrogen atmosphere. The reaction was poured into ice-water (30 mL) and stirred for 3 min. The aqueous phase was extracted with ethyl acetate (10 mL×3). The combined organic phase was washed with brine (10 mL×3), dried with anhydrous sodium sulfate, filtered and concentrated in vacuum. The residue was purified by prep-HPLC (column: Unisil 3-100 C18 Ultra 150*50 mm*3 um; mobile phase: [water (formic acid)-acetonitrile]; B %: 46%-76%, 7 min) to afford tert-butyl 4-[[2-[[(1S)-1-[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]amino]-2-oxo-ethoxy]methyl]piperidine-1-carboxylate (9 mg, 3%) as a white solid. MS (ESI) m/z: 714.4 [M+1]⁺.

Step 8: Preparation of (2S,4R)-1-[(2S)-3,3-dimethyl-2-[[2-(4-piperidyl methoxy)acetyl]amino]butanoyl]-N-[(1S)-1-[4-(4-ethylthiazol-5-yl)phenyl]ethyl]-4-hydroxy-pyrrolidine-2-carboxamide

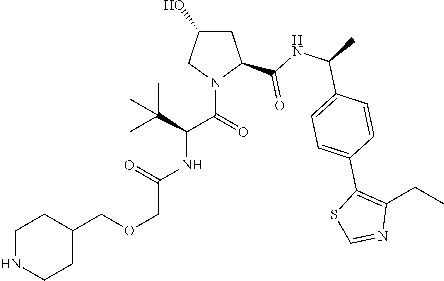 [SMILES: CCc1ncsc1-c1ccc([C@H](C)NC(=O)[C@@H]2C[C@@H](O)CN2C(=O)[C@@H](NC(=O)COCC2CCNCC2)C(C)(C)C)cc1]

To a solution of tert-butyl 4-[[2-[[(1S)-1-[[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]amino]-2-oxo-ethoxy]methyl]piperidine-1-carboxylate (9 mg, 0.01 mmol) in dichloromethane (2 mL) was added hydrochloric acid in dioxane (4 M, 0.03 mL). The mixture was stirred at 20° C. for 2 h, then concentrated in vacuum to afford (2S,4R)-1-[(2S)-3,3-dimethyl-2-[[2-(4-piperidylmethoxy)acetyl]amino]butanoyl]-N-[(1S)-1-[4-(4-ethylthiazol-5-yl)phenyl]ethyl]-4-hydroxy-pyrrolidine-2-carboxamide (8 mg, crude, hydrochloridesalt) as a white solid.

Step 9: Preparation of tert-butyl 3-[7-(8-ethyl-3-hydroxy-1-naphthyl)-2-[2-[[4-[[2-[[(1S)-1-[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]amino]-2-oxo-ethoxy]methyl]-1-piperidyl]ethoxy]-8-fluoro-pyrido[4,3-d]pyrimidin-4-yl]-3,8-diazabicyclo[3.2.1]octane-8-carboxylate

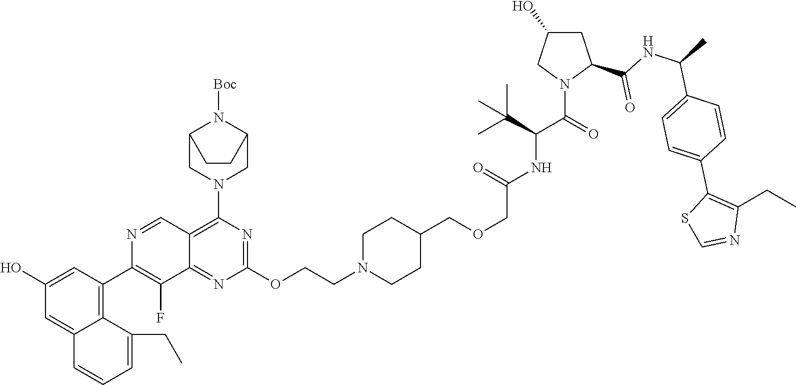 [SMILES: CCc1ncsc1-c1ccc([C@H](C)NC(=O)[C@@H]2C[C@@H](O)CN2C(=O)[C@@H](NC(=O)COCC2CCN(CCOc3nc(N4CC5CCC(C4)N5C(=O)OC(C)(C)C)c4cnc(-c5cc(O)cc6cccc(CC)c56)c(F)c4n3)CC2)C(C)(C)C)cc1]

To a solution of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (9 mg, 0.02 mmol) in dichloromethane (2 mL) was added N-methyl morpholine (6 mg, 0.06 mmol, 0.01 mL), the mixture was stirred at 25° C. (2S,4R)-1-[(2S)-3,3-dimethyl-2-[[2-(4-piperidylmethoxy)acetyl]amino]butanoyl]-N-[(1S)-1-[4-(4-ethylthiazol-5-yl)phenyl]ethyl]-4-hydroxy-pyrrolidine-2-carboxamide (8 mg, 0.01 mmol, hydrochloride) was added to the mixture, the mixture was stirred for 0.5 h. Then sodium triacetoxyborohydride (5.0 mg, 25 umol) was added, the reaction mixture was stirred at 25° C. for 1.5 h. The reaction mixture was diluted with water (20 mL) and extracted with dichloromethane (10 mL×3). The organic layer was washed with brine (20 mL×3), dried over anhydrous sodium sulfate, filtered, and concentrated in vacuum to afford tert-butyl 3-[7-(8-ethyl-3-hydroxy-1-naphthyl)-2-[2-[[4-[[2-[[(1S)-1-[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]amino]-2-oxo-ethoxy]methyl]-1-piperidyl]ethoxy]-8-fluoro-pyrido[4,3-d]pyrimidin-4-yl]-3,8-diazabicyclo[3.2.1]octane-8-carboxylate (11 mg, 75%) as a yellow solid. MS (ESI) m/z: 1185.9 [M+1]⁺.

Step 10: Preparation of (2S,4R)-1-[(2S)-2-[[2-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-N-[(1S)-1-[4-(4-ethylthiazol-5-yl)phenyl]ethyl]-4-hydroxy-pyrrolidine-2-carboxamide

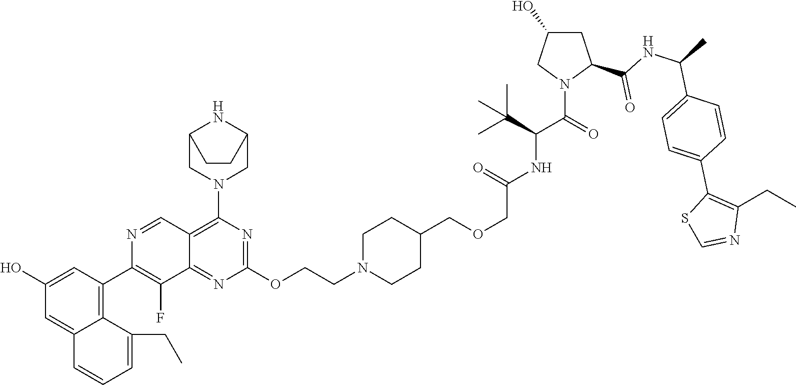 [SMILES: CCc1ncsc1-c1ccc([C@H](C)NC(=O)[C@@H]2C[C@@H](O)CN2C(=O)[C@@H](NC(=O)COCC2CCN(CCOc3nc(N4CC5CCC(C4)N5)c4cnc(-c5cc(O)cc6cccc(CC)c56)c(F)c4n3)CC2)C(C)(C)C)cc1]

To a solution of tert-butyl 3-[7-(8-ethyl-3-hydroxy-1-naphthyl)-2-[2-[[4-[2-[[(1S)-1-[(2S,4R)-2-[[(1S)-1-[4-(4-ethylthiazol-5-yl)phenyl]ethyl]carbamoyl]-4-hydroxy-pyrrolidine-1-carbonyl]-2,2-dimethyl-propyl]amino]-2-oxo-ethoxy]methyl]-1-piperidyl]ethoxy]-8-fluoro-pyrido[4,3-d]pyrimidin-4-yl]-3,8-diazabicyclo[3.2.1]octane-8-carboxylate (11 mg, 0.01 mmol) in dichloromethane (3 mL) was added formic acid (1.22 g, 26 mmol, 1 mL). The mixture was stirred at 25° C. for 1 h. The reaction mixture was diluted with water (40 mL) and extracted with ethyl acetate (50 mL×3). The organic layer was washed with brine (50 mL×3), dried over sodium sulfate, filtered, and concentrated in vacuum. The residue was purified by prep-HPLC (column: Unisil 3-100 C18 Ultra 150*50 mm*3 um; mobile phase: [water (formic acid)-acetonitrile]; B %: 13%-43%, 7 min) to afford (2S,4R)-1-[(2S)-2-[[2-[[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-piperidyl]methoxy]acetyl]amino]-3,3-dimethyl-butanoyl]-N-[(1S)-1-[4-(4-ethylthiazol-5-yl)phenyl]ethyl]-4-hydroxy-pyrrolidine-2-carboxamide (8 mg, 76%) as green solid. MS (ESI) m/z: 1085.4 [M+1]⁺; ¹H NMR (400 MHz, MeOD-d₄) δ 9.11 (s, 1H), 8.91-8.84 (m, 1H), 7.63 (d, J=8.0 Hz, 1H), 7.42-7.33 (m, 5H), 7.30 (d, J=2.4 Hz, 1H), 7.17 (d, J=7.2 Hz, 1H), 7.01 (d, J=2.4 Hz, 1H), 4.58 (s, 3H), 4.44 (s, 1H), 4.14-3.80 (m, 9H), 3.77-3.72 (m, 1H), 3.70-3.63 (m, 2H), 3.51-3.45 (m, 4H), 3.08-2.96 (m, 2H), 2.80 (q, J=7.6 Hz, 2H), 2.42-2.16 (m, 4H), 2.14-1.91 (m, 9H), 1.70-1.55 (m, 3H), 1.47 (dd, J=1.6, 6.8 Hz, 2H), 1.40-1.34 (m, 2H), 1.29-1.23 (m, 3H), 1.06-0.97 (m, 9H), 0.89 (t, J=7.2 Hz, 3H).

Exemplary Synthesis of (2S,4R)-1-[(2R)-2-{3-[(1-{2-[(4-{3,8-diazabicyclo[3.2.1]octan-3-yl}-7-(8-ethynyl-7-fluoro-3-hydroxynaphthalen-1-yl)-8-fluoropyrido[4,3-d]pyrimidin-2-yl)oxy]ethyl}-4-methylpiperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 313)

Step 1: Preparation of tert-butyl 3-[8-fluoro-7-[7-fluoro-3-hydr oxy-8-(2-triisopropylsilylethynyl)-1-naphthyl]-2-[2-[[4-[5-[(1R)-1-[(2S,4R)-4-hydroxy-2-[[(1S)-1-[4-(4-methylthiazol-5-yl)phenyl]ethyl]carbamoyl]pyrrolidine-1-car bonyl]-isobutyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate

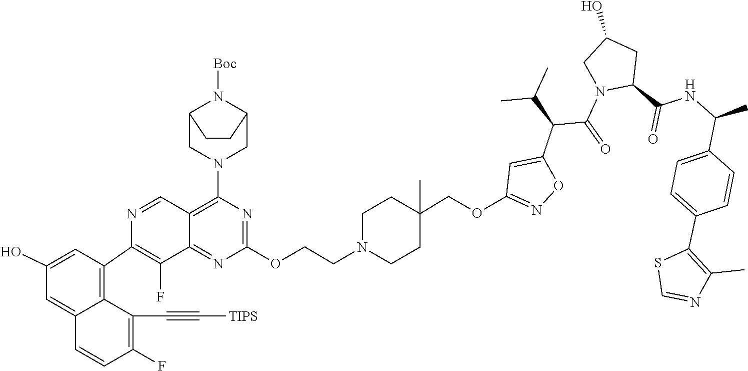

To a solution of tert-butyl 3-[8-fluoro-7-[7-fluoro-3-hydroxy-8-(2-triisopropylsilyl ethynyl)-1-naphthyl]-2-(2-oxoethoxy)pyrido[4,3-d]pyrimidin-4-yl]-3,8-diazabicyclo[3.2.1]octane-8-carboxylate (178 mg, 0.2 mmol) and (2S,4R)-4-hydroxy-1-[(2R)-3-methyl-2-[3-[(4-methyl-4-piperidyl)methoxy]isoxazol-5-yl]butanoyl]-N-[(1S)-1-[4-(4-methylthiazol-5-yl)phenyl]ethyl]pyrrolidine-2-carboxamide (143 mg, 0.2 mmol) in CH₂Cl₂ (5.0 mL) and i-PrOH (5.0 mL) were added acetic acid (0.05 mL, 0.9 mmol) and 2-picoline borane complex (75 mg, 0.7 mmol). The mixture was stirred at 25° C. for 1 h, then concentrated under reduced pressure. The residue was purified by flash column (0-10% methanol in dichloromethane) to afford tert-butyl 3-[8-fluoro-7-[7-fluoro-3-hydroxy-8-(2-triisopropylsilylethynyl)-1-naphthyl]-2-[2-[[4-[5-[(1R)-1-[(2S,4R)-4-hydroxy-2-[[(1S)-1-[4-(4-methylthiazol-5-yl)phenyl]ethyl]carbamoyl]pyr rolidine-1-carbonyl]-isobutyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate (200 mg, 75%) as yellow solid. MS (ESI) m/z: 1351.7 [M+1]⁺.

Step 2: Preparation of tert-butyl 3-[7-(8-ethynyl-7-fluoro-3-hydr oxy-1-naphthyl)-8-fluoro-2-[2-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate

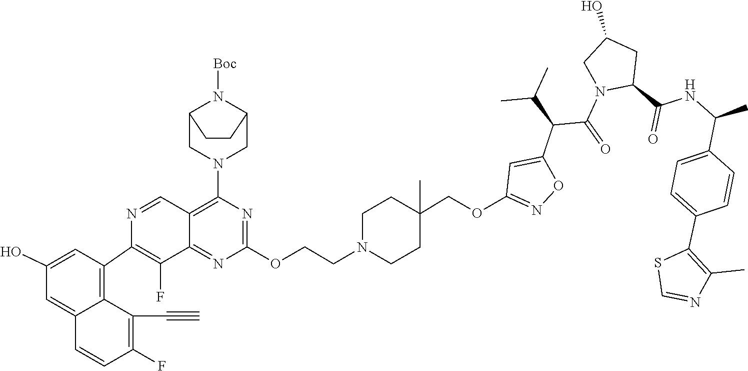 [SMILES: C#Cc1c(F)ccc2cc(O)cc(-c3ncc4c(N5CC6CCC(C5)N6C(=O)OC(C)(C)C)nc(OCCN5CCC(C)(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a solution of tert-butyl 3-[8-fluoro-7-[7-fluoro-3-hydroxy-8-(2-triisopropylsilyl ethynyl)-1-naphthyl]-2-[2-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate (200 mg, 0.1 mmol) in DMF (3.0 mL) was added CsF (112 mg, 0.7 mmol). The mixture was stirred at 25° C. for 4 h, then diluted with water (5.0 mL) and filtered. The filter cake was washed with CH₂Cl₂ (10 ml×3), the filtrate was concentrated to afford tert-butyl 3-[7-(8-ethynyl-7-fluoro-3-hydroxy-1-naphthyl)-8-fluoro-2-[2-[[4-[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate (176 mg) as yellow solid, which was used directly in the next step. MS (ESI) m/z: 1195.4 [M+1]⁺.

Step 3: Preparation of (2S,4R)-1-[(2R)-2-[3-[[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-methyl-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide

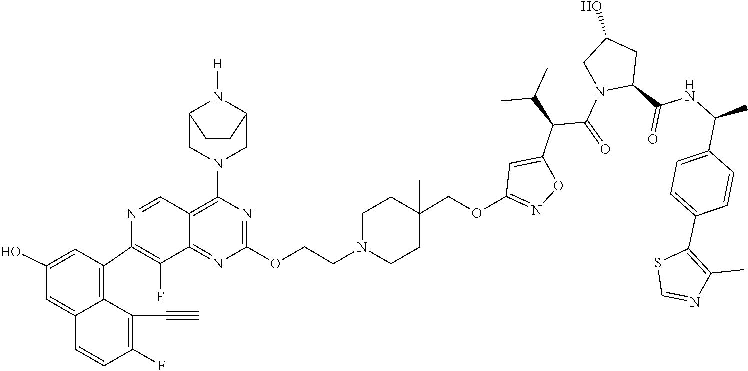 [SMILES: C#Cc1c(F)ccc2cc(O)cc(-c3ncc4c(N5CC6CCC(C5)N6)nc(OCCN5CCC(C)(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

A solution of tert-butyl 3-[7-(8-ethynyl-7-fluoro-3-hydroxy-1-naphthyl)-8-fluoro-2-[2-[[4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-2-methyl-propyl]isoxazol-3-yl]oxymethyl]-4-methyl-1-piperidyl]ethoxy]pyrido[4,3-d]pyrimidin-4-yl]-3,8-diazabicyclo[3.2.1]octane-8-carboxylate (176 mg, 0.1 mmol) in CH₂Cl₂ (3 mL) and 4 M HCl in dioxane (1.0 mL) was stirred at 25° C. for 20 min. The reaction was suspended in petroleum ether (5.0 mL). The precipitates were collected by filtration, re-dissolved in H₂O (5.0 ml), treated with triethylamine (0.1 mL) and extracted with CH₂Cl₂ (20 mL). The organic phase was dried over anhydrous Na₂SO₄, filtered, and concentrated under reduced pressure. The residue was purified by preparative HPLC (Column: ChiralPak IH, 250*30 mm, 10 um; mobile phase: [acetonitrile/ethanol (0.1% NH₃H₂O)]; B %: 25%; Gradient time: 6 min) to afford (2S,4R)-1-[(2R)-2-[3-[1-[2-[[4-(3,8-diazabicyclo[3.2.1]octan-3-yl)-7-(8-ethynyl-7-fluoro-3-hydroxy-1-naphthyl)-8-fluoro-pyrido[4,3-d]pyrimidin-2-yl]oxyethyl]-4-methyl-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (72.2 mg, 43%) as yellow solid. MS (ESI) m/z: 1095.2 [M+1]⁺; 1H NMR (400 MHz, CD₃OD) δ 9.01 (s, 1H), 8.90-8.85 (m, 1H), 7.87-7.84 (m, 1H), 7.47-7.37 (m, 4H), 7.36-7.29 (m, 2H), 7.24-7.19 (m, 1H), 6.01 (d, J=2.0 Hz, 1H), 5.06-5.00 (m, 1H), 4.63-4.40 (m, 8H), 3.97 (s, 2H), 3.83 (d, J=4.0, Hz, 1H), 3.77-3.60 (m, 6H), 3.37 (s, 1H), 2.94 (t, J=5.2 Hz, 2H), 2.84-2.74 (m, 2H), 2.69-2.57 (m, 2H), 2.502.46 (m, 3H), 2.40-2.17 (m, 2H), 1.94-1.70 (m, 7H), 1.52 (d, J=7.2 Hz, 3H), 1.08 (s, 3H), 1.05 (d, J=6.4 Hz, 3H), 0.91-0.87 (m, 3H).

Exemplary Synthesis of (2S,4R)—N-[(1S)-1-[3-chloro-4-(4-methyl-1,3-thiazol-5-yl)phenyl]ethyl]-1-[(2S)-2-{2-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}piperidin-4-yl)methoxy]acetamido}-3,3-dimethylbutanoyl]-4-hydroxypyrrolidine-2-carboxamide (Compound 332)

Step 1: Preparation of tert-butyl 4-((2-(((S)-1-((2S,4R)-2-(((S)-1-(3-chloro-4-(4-methylthiazol-5-yl)phenyl)ethyl)carbamoyl)-4-hydroxypyrrolidin-1-yl)-3,3-dimethyl-1-oxobutan-2-yl)amino)-2-oxoethoxy)methyl)piperidine-1-carboxylate

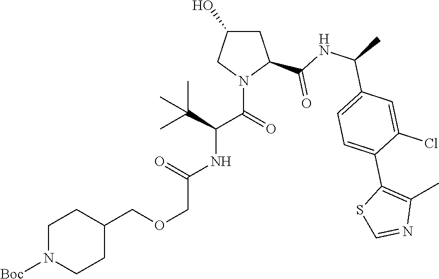 [SMILES: Cc1ncsc1-c1ccc([C@H](C)NC(=O)[C@@H]2C[C@@H](O)CN2C(=O)[C@@H](NC(=O)COCC2CCN(C(=O)OC(C)(C)C)CC2)C(C)(C)C)cc1Cl]

To a solution of (2S,4R)-1-((S)-2-amino-3,3-dimethylbutanoyl)-N—((S)-1-(3-chloro-4-(4-methylthiazol-5-yl)phenyl)ethyl)-4-hydroxypyrrolidine-2-carboxamide (350 mg, 0.7 mmol, hydrochloride) and N,N-diisopropylethylamine (878 mg, 7 mmol, 1.2 mL) in N,N-dimethylformamide (7 mL) was added 2-((1-(tert-butoxycarbonyl) piperidin-4-yl) methoxy) acetic acid (557 mg, 2 mmol), 1-hydroxybenzotriazole (275 mg, 2 mmol) and 1-(3-dimethylaminopropyl)-3-ethylcarbodiimide hydrochloride (390 mg, 2 mmol). The mixture was stirred at 25° C. for 12 h. The mixture was filtered. The residue was purified by semi-preparative reverse phase HPLC (column: Phenomenex luna C18 150*40 mm*15 um; mobile phase: [water (formic acid)-acetonitrile]; B %: 45%-75%, 10 min) to afford tert-butyl 4-((2-(((S)-1-((2S,4R)-2-(((S)-1-(3-chloro-4-(4-methylthiazol-5-yl)phenyl)ethyl)carbamoyl)-4-hydroxypyrrolidin-1-yl)-3,3-dimethyl-1-oxobutan-2-yl)amino)-2-oxoethoxy)methyl)piperidine-1-carboxylate (165 mg, 32%) as a colorless gum. MS (ESI) m/z: 734.1 [M+1]⁺.

Step 2: Preparation of (2S,4R)—N—((S)-1-(3-chloro-4-(4-methylthiazol-5-yl)phenyl)ethyl)-1-((S)-3,3-dimethyl-2-(2-(piperidin-4-ylmethoxy) acetamido)butanoyl)-4-hydroxypyrrolidine-2-carboxamide

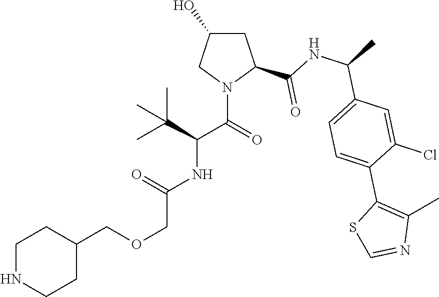 [SMILES: Cc1ncsc1-c1ccc([C@H](C)NC(=O)[C@@H]2C[C@@H](O)CN2C(=O)[C@@H](NC(=O)COCC2CCNCC2)C(C)(C)C)cc1Cl]

To a solution of tert-butyl 4-((2-(((S)-1-((2S,4R)-2-(((S)-1-(3-chloro-4-(4-methylthiazol-5-yl)phenyl)ethyl)carbamoyl)-4-hydroxypyrrolidin-1-yl)-3,3-dimethyl-1-oxobutan-2-yl)amino)-2-oxoethoxy)methyl)piperidine-1-carboxylate (190 mg, 0.3 mmol) in dichloromethane (3 mL) was added hydrochloric acid/dioxane (4 M, 3 mL). The mixture was stirred at 25° C. for 2 h, then concentrated under reduced pressure. The residue was purified by semi-preparative reverse phase HPLC (column: Unisil 3-100 C18 Ultra 150*50 mm*3 um; mobile phase: [water (formic acid)-acetonitrile]; B %: 12%-42%, 7 min) to afford (2S,4R)—N—((S)-1-(3-chloro-4-(4-methylthiazol-5-yl)phenyl)ethyl)-1-((S)-3,3-dimethyl-2-(2-(piperidin-4-ylmethoxy) acetamido)butanoyl)-4-hydroxypyrrolidine-2-carboxamide (130 mg, 74%, formic acid salt) as a white solid. MS (ESI) m/z: 634.1 [M+1]⁺.

Step 3: Preparation of tert-butyl(1R,5S)-3-(2-(2-(4-((2-(((S)-1-((2S,4R)-2-(((S)-1-(3-chloro-4-(4-methylthiazol-5-yl)phenyl)ethyl)carbamoyl)-4-hydroxypyrrolidin-1-yl)-3,3-dimethyl-1-oxobutan-2-yl)amino)-2-oxoethoxy)methyl) piperidin-1-yl) ethoxy)-7-(8-ethyl-3-hydroxynaphthalen-1-yl)-8-fluoropyrido[4,3-d]pyrimidin-4-yl)-3,8-diazabicyclo[3.2.1]octane-8-carboxylate

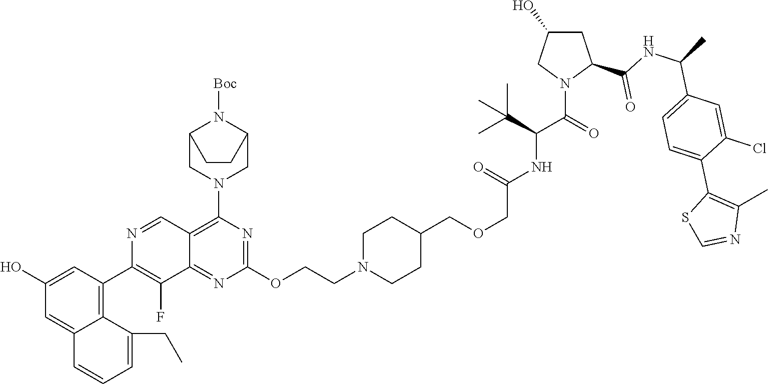 [SMILES: CCc1cccc2cc(O)cc(-c3ncc4c(N5CC6CCC(C5)N6C(=O)OC(C)(C)C)nc(OCCN5CCC(COCC(=O)N[C@H](C(=O)N6C[C@H](O)C[C@H]6C(=O)N[C@@H](C)c6ccc(-c7scnc7C)c(Cl)c6)C(C)(C)C)CC5)nc4c3F)c12]

To a solution of (2S,4R)—N—((S)-1-(3-chloro-4-(4-methylthiazol-5-yl)phenyl)ethyl)-1-((S)-3,3-dimethyl-2-(2-(piperidin-4-ylmethoxy) acetamido)butanoyl)-4-hydroxypyrrolidine-2-carboxamide formate (90 mg, 0.1 mmol) in dimethyl sulfoxide (1 mL) and 1,2-dichloroethane (1 mL) was added 4-methylmorpholine (40 mg, 0.4 mmol) and tert-butyl(1R,5S)-3-(7-(8-ethyl-3-hydroxynaphthalen-1-yl)-8-fluoro-2-(2-oxoethoxy)pyrido[4,3-d]pyrimidin-4-yl)-3,8-diazabicyclo[3.2.1]octane-8-carboxylate (78 mg, 0.1 mmol). The mixture was stirred at 25° C. for 0.5 h. Then sodium triacetoxyborohydride (84 mg, 0.4 mmol) was added and the mixture was stirred at 25° C. for another 3 h. The mixture was poured into water (50 mL). The aqueous phase was extracted with dichloromethane (50 mL×2). The combined organic phase was washed with brine (40 mL×2), dried with anhydrous sodium sulfate, filtered, and concentrated in vacuum. The residue was purified by prep-thin-layer chromatography (dichloromethane/methanol=10:1) to afford tert-butyl(1R,5S)-3-(2-(2-(4-((2-(((S)-1-((2S,4R)-2-(((S)-1-(3-chloro-4-(4-methylthiazol-5-yl)phenyl)ethyl)carbamoyl)-4-hydroxypyrrolidin-1-yl)-3,3-dimethyl-1-oxobutan-2-yl)amino)-2-oxoethoxy)methyl) piperidin-1-yl) ethoxy)-7-(8-ethyl-3-hydroxynaphthalen-1-yl)-8-fluoropyrido[4,3-d]pyrimidin-4-yl)-3,8-diazabicyclo[3.2.1]octane-8-carboxylate (125 mg, crude) as a white solid. MS (ESI) m/z: 1205.2 [M+1]⁺.

Step 4: Preparation of (2S,4R)-1-((S)-2-(2-((1-(2-((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)ethyl)piperidin-4-yl)methoxy) acetamido)-3,3-dimethylbutanoyl)-N—((S)-1-(3-chloro-4-(4-methylthiazol-5-yl)phenyl)ethyl)-4-hydroxypyrrolidine-2-carboxamide

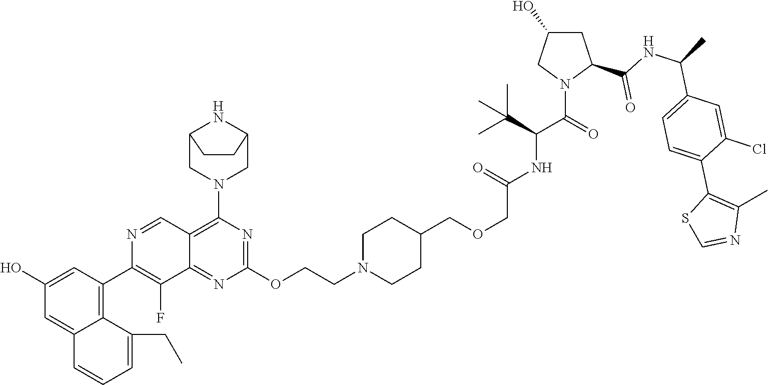 [SMILES: CCc1cccc2cc(O)cc(-c3ncc4c(N5CC6CCC(C5)N6)nc(OCCN5CCC(COCC(=O)N[C@H](C(=O)N6C[C@H](O)C[C@H]6C(=O)N[C@@H](C)c6ccc(-c7scnc7C)c(Cl)c6)C(C)(C)C)CC5)nc4c3F)c12]

To a solution of tert-butyl(1R,5S)-3-(2-(2-(4-((2-(((S)-1-((2S,4R)-2-(((S)-1-(3-chloro-4-(4-methylthiazol-5-yl)phenyl)ethyl)carbamoyl)-4-hydroxypyrrolidin-1-yl)-3,3-dimethyl-1-oxobutan-2-yl)amino)-2-oxoethoxy)methyl) piperidin-1-yl) ethoxy)-7-(8-ethyl-3-hydroxynaphthalen-1-yl)-8-fluoropyrido[4,3-d]pyrimidin-4-yl)-3,8-diazabicyclo[3.2.1]octane-8-carboxylate (115 mg, 0.1 mmol) in dichloromethane (5 mL) was added trifluoroacetic acid (3.08 g, 27 mmol, 2 mL). The mixture was stirred at 25° C. for 3 h, then concentrated under reduced pressure. The residue was purified by semi-preparative reverse phase HPLC (column: Unisil 3-100 C18 Ultra 150*50 mm*3 um; mobile phase: [water (formic acid)-acetonitrile]; B %: 13%-43%, 7 min) to afford (2S,4R)-1-((S)-2-(2-((1-(2-((4-((1R,5S)-3,8-diazabicyclo[3.2.1]octan-3-yl)-7-(8-ethyl-3-hydroxynaphthalen-1-yl)-8-fluoropyrido[4,3-d]pyrimidin-2-yl)oxy)ethyl) piperidin-4-yl)methoxy) acetamido)-3,3-dimethylbutanoyl)-N—((S)-1-(3-chloro-4-(4-methylthiazol-5-yl)phenyl)ethyl)-4-hydroxypyrrolidine-2-carboxamide (44.4 mg, 41%) as a white solid. MS (ESI) m/z: 1105.4 [M+1]⁺; 1H NMR (400 MHz, DMSO-d₆) δ 9.92 (br s, 1H), 9.13 (s, 1H), 9.09 (s, 1H), 8.49 (d, J=7.2 Hz, 1H), 8.15 (s, 1H), 7.67 (d, J=8.0 Hz, 1H), 7.50 (s, 1H), 7.42 (d, J=8.0 Hz, 1H), 7.37 (t, J=7.6 Hz, 1H), 7.33-7.26 (m, 3H), 7.13 (d, J=7.2 Hz, 1H), 6.97 (d, J=2.8 Hz, 1H), 5.14 (br s, 1H), 4.94-4.85 (m, 1H), 4.62-4.47 (m, 5H), 4.43 (br t, J=8.0 Hz, 1H), 4.29 (br s, 1H), 3.95-3.86 (m, 4H), 3.82-3.76 (m, 1H), 3.74-3.67 (m, 1H), 3.63-3.54 (m, 2H), 3.33 (br d, J=6.0 Hz, 2H), 3.06-2.97 (m, 2H), 2.77 (br d, J=5.2 Hz, 2H), 2.31-2.16 (m, 6H), 2.15-2.04 (m, 3H), 1.82 (br s, 3H), 1.77-1.58 (m, 4H), 1.36 (br d, J=7.2 Hz, 3H), 1.28-1.17 (m, 2H), 0.93 (s, 9H), 0.82 (t, J=7.2 Hz, 3H).

Exemplary Synthesis of (2S,4R)-1-[(2R)-2-{3-[(1-{2-[(4-{3,8-diazabicyclo[3.2.1]octan-3-yl}-7-(8-ethyl-3-hydroxynaphthalen-1-yl)-8-fluoropyrido[4,3-d]pyrimidin-2-yl)oxy]ethyl}-4-fluoropiperidin-4-yl)methoxy]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Compound 337)

Step 1: Preparation of tert-butyl 3-[7-(8-ethyl-3-hydroxy-1-naphthyl)-8-fluoro-2-[2-[[4-fluoro-4-[[5-[(1R)-1-[(2S,4R)-4-hydroxy-2-[[(1S)-1-[4-(4-methylthiazol-5-yl)phenyl]ethyl]carbamoyl]pyrrolidine-1-carbonyl]-isobutyl]isoxazol-3-yl]oxymethyl]-1-piperidyl]ethoxy]pyrido[4,3-d]pyrimidin-4-yl]-3,8-diazabicyclo[3.2.1]octane-8-carboxylate

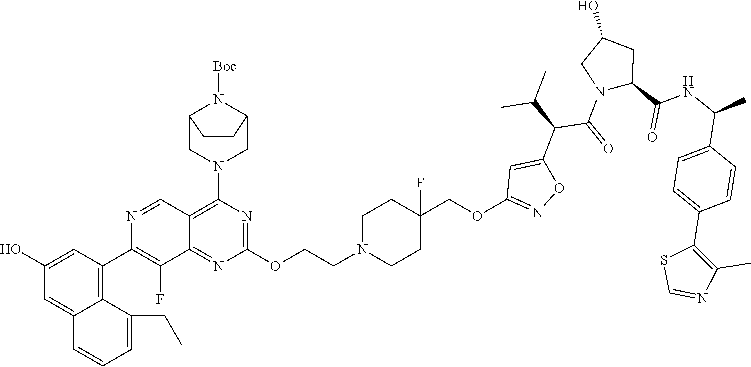 [SMILES: CCc1cccc2cc(O)cc(-c3ncc4c(N5CC6CCC(C5)N6C(=O)OC(C)(C)C)nc(OCCN5CCC(F)(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a reaction mixture of tert-butyl 3-[7-(8-ethyl-3-hydroxy-1-naphthyl)-8-fluoro-2-(2-oxoethoxy)pyrido[4,3-d]pyrimidin-4-yl]-3,8-diazabicyclo[3.2.1]octane-8-carboxylate (300 mg, 0.4 mmol) and (2S,4R)-1-[(2R)-2-[3-[(4-fluoro-4-piperidyl)methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (219 mg, 0.4 mmol) in CH₂Cl₂ (6.0 mL) and i-PrOH (1.0 mL) was added HOAc (86.4 mg, 1.4 mmol) and 2-picoline borane complex (190 mg, 1.8 mmol). The resulting suspension was stirred at 25° C. for 1 h, concentrated in vacuum and purified by column chromatography (0-6% then up to 8% methanol in dichloromethane) to afford tert-butyl 3-[7-(8-ethyl-3-hydroxy-1-naphthyl)-8-fluoro-2-[2-[[4-fluoro-4-[5-[(1R)-1-[(2S,4R)-4-hydroxy-2-[[(1S)-1-[4-(4-methylthiazol-5-yl)phenyl]ethyl]carbamoyl]pyrroledine-1-carbonyl]-isobutyl]isoxazol-3-yl]oxymethyl]-1-piperidyl]ethoxy]pyrido[4,3-d]pyrimidin-4-yl]-3,8-diazabicyclo[3.2.1]octane-8-carboxylate (310 mg, 70%) as yellow solid. MS (ESI) m/z: 1185.5 [M+H]⁺.

Step 2: Preparation of (2S,4R)-1-[(2R)-2-[3-[[1-[2-[[4-(3,8-diaza bicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrroledine-2-carboxamide

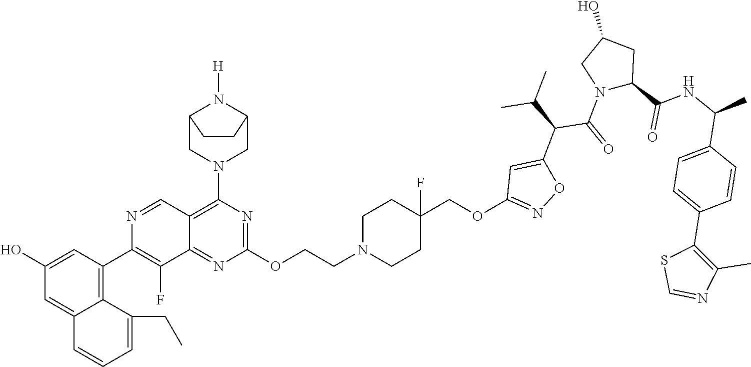 [SMILES: CCc1cccc2cc(O)cc(-c3ncc4c(N5CC6CCC(C5)N6)nc(OCCN5CCC(F)(COc6cc([C@H](C(=O)N7C[C@H](O)C[C@H]7C(=O)N[C@@H](C)c7ccc(-c8scnc8C)cc7)C(C)C)on6)CC5)nc4c3F)c12]

To a reaction mixture of tert-butyl 3-[7-(8-ethyl-3-hydroxy-1-naphthyl)-8-fluoro-2-[2-[4-fluoro-4-[[5-[(1R)-1-[(2S,4R)-4-hydroxy-2-[[(1S)-1-[4-(4-methylthiazol-5-yl) phen yl]ethyl]carbamoyl]pyrrolidine-1-carbonyl]-isobutyl]isoxazol-3-yl]oxymethyl]-1-piperidyl]ethoxy]pyrido[4,3-d]pyrimidin-4-yl]-3,8-diazabicyclo[3.2.1]octane-8-carboxylate (310 mg, 0.2 mmol) in dichloromethane (3.0 mL) was added 4 M HCl in dioxane (3.0 mL), the resulting suspension was stirred at 20° C. for 10 min. the mixture was suspended in petroleum ether (30 mL), the solid was collected by filtration, re-dissolved in THF (30 mL), adjusted the pH to 8 with triethylamine and filtered again. The filtrate was concentrated under reduced pressure and the residue was purified by preparative HPLC (Column: Phenomenex C18 75*30 mm*3 um; Mobile phase: [water (formic acid)-acetonitrile]; B %: 8-38; Gradient Time: 25 min; 100% B Hold Time: 3 min; Flow rate: 25 mL/min) to afford (2S,4R)-1-[(2R)-2-[3-[1-[2-[[4-(3,8-diazabicyclo[3.2.1]octan-3-yl)-7-(8-ethyl-3-hydroxy-1-naphthyl)-8-fluoro-pyrido[4,3-d]pyrimidin-2-yl]oxyethyl]-4-fluoro-4-piperidyl]methoxy]isoxazol-5-yl]-3-methyl-butanoyl]-4-hydroxy-N-[(1S)-1-[4-(4-methylthiazol-5-yl)phenyl]ethyl]pyrrolidine-2-carboxamide (193.3 mg, formic acid salt, 68%) as white solid. MS (ESI) m/z: 1085.9 [M+H]⁺; ¹H NMR (400 MHz, DMSO-d₆) δ 9.10 (s, 1H), 9.01-8.80 (m, 1H), 8.43 (d, J=7.6 Hz, 1H), 7.66 (d, J=8.0 Hz, 1H), 7.49-7.40 (m, 2H), 7.39-7.32 (m, 3H), 7.28 (d, J=2.4 Hz, 1H), 7.11 (d, J=7.2 Hz, 1H), 6.97 (d, J=2.4 Hz, 1H), 6.19-5.96 (m, 1H), 4.91 (quint, J=7.2 Hz, 1H), 4.58-4.41 (m, 5H), 4.37 (t, J=7.6 Hz, 1H), 4.31-4.15 (m, 5H), 3.77-3.60 (m, 7H), 2.84-2.72 (m, 4H), 2.45 (s, 3H), 2.36-2.28 (m, 2H), 2.28-2.15 (m, 3H), 2.07-1.98 (m, 1H), 1.90-1.66 (m, 9H), 1.46-1.35 (m, 3H), 1.00-0.92 (m, 3H), 0.84-0.76 (m, 6H).

Example 3: Biological Assays

Reagents. SW1573 (CRL-2170), AsPC-1 (CRL-1682), and SW620 (CCL-227) cells were purchased from ATCC. SW1573 cells are homozygous for the G12C mutation in KRAS, AsPC-1 cells are homozygous for the G12D mutation, and SW620 cells are homozygous for the G12V mutation. SW1573 cells were cultured in DMEM+F12+Glutamax (+Sodium Bicarbonate, +sodium pyruvate, ThermoFisher scientific, #10565) supplemented with 10% FBS (ThermoFisher scientific, #2060357) and 1% Antibiotic-Antimycotic (ThermoFisher scientific, #15240096). AsPC-1 cells were cultured in GIBCO RPMI-1640 (ThermoFisher scientific, catalog #61870-036 with Glutamax, No HEPES, No Na Pyruvate) supplemented with 10% FBS (ThermoFisher scientific, catalog #2060357) and 1× Penicillin-Streptomycin (ThermoFisher scientific, catalog #15140122, 10000 U/ml, 100×). SW620 cells were cultured in DMEM Glutamax (ThermoFisher scientific, catalog #10566016) supplemented with 10% FBS (ThermoFisher scientific, catalog #2060357) and 1× Penicillin-Streptomycin (ThermoFisher scientific, catalog #15140122, 10000 U/ml, 100×). For degradation assays, 384 well plates were used. Alamar blue was purchased from ThermoFisher scientific (A50101) and used according to manufacturer instructions. The Nano-Glo® HiBiT Lytic Detection System and Nano-Glo® HiBiT Blotting System were purchased from Promega (#N3050 and #N2410) and used according to manufacturer instructions.

HiBiT-tagged cell line generation. To create the SW1573 HiBiT-KRASG12C cell line, the nucleotide sequence encoding the 11 amino acid HiBiT tag from Promegal was appended to the N-terminus of the KRASG12C coding sequence. This synthesized DNA was cloned into a modified pcDNA3.1 vector under the control of the thymidine kinase (TK) promoter. This HiBiT-KRASG12C expressing plasmid was transfected into parental SW1573 cells and colonies were selected using 400 μg/mL zeocin. The resulting clonal lines were screened for expression of HiBiT-KRASG12C protein using Nano-Glo® HiBiT Blotting System. A luminescent band of the correct size for HiBiT-KRASG12C was detected in several clones. Clone E6 was selected for further studies.

To create the AsPC-1 HiBiT-KRASG12D cell line, CRISPR-Cas9 was used to insert the HiBiT tag onto the N-terminus of one or more of the endogenous KRAS alleles. See Schwinn, M. K. et al. (2018) CRISPR-mediated tagging of endogenous proteins with a luminescent peptide. ACS Chem. Biol. 13 (2), 467-474. After knock-in of the tag, individual clones were isolated by single cell dilution and screened for HiBiT insertion by PCR. A clone was identified that contained the desired HiBiT-KRASG12D sequence at one allele while the remaining two KRASG12D alleles were untagged. This clone was used in further studies.

HiBiT degradation assays (G12C and G12D). Either SW1573 HiBiT-KRasG12C or AsPC-1 HiBiT-KRasG12D cells were plated at 9000 or 15,000 cells/well in a 384 well plate (Corning #3770). Compounds of the present disclosure and positive controls were diluted in the appropriate cell media and applied to the plated cells resulting in a final concentration titration of 10 μM to 508 pM in 0.5% DMSO. Gambogic acid was used as a positive control for cytotoxicity and titrated from 10 μM to 78 nM. All assays were performed in triplicate. Cells were treated with compounds for 24 hours at 37° C. in an incubator containing 5% CO₂. Following treatment, alamar blue was used to determine if any compounds led to a loss in cell viability after 24 hours. The 10× alamar blue reagent was added to a final concentration of 1× as recommended by the manufacturer and cells were returned to the incubator for 4 hours. Plates were then allowed to equilibrate to room temperature and fluorescence was measured using a Perkin-Elmer EnVision. After completion of the fluorescence read, the liquid from each well was removed and the cells were washed with PBS. The Nano-Glo® HiBiT detection reagent was prepared according to the manufacturer's instructions and added to each well after the PBS wash was removed. Following incubation at room temperature for 45 minutes, the luminescence was read using the ultra-sensitive luminescence aperture on the Envision instrument.

Data analysis. Fluorescence values from the alamar blue treatment were normalized to the DMSO-only control for each compound titration. Without wishing to be bound by theory, it was hypothesized that compounds that led to a ≥50% loss in cell viability of the KRAS-independent SW1573 HiBiT-KRasG12C cells were likely generally cytotoxic and loss of HiBiT-KRASG12C signal from these compounds may be due to lower cell number and not specific KRASG12C degradation. These points were therefore excluded from the data analysis for degradation. HiBiT luminescence was normalized to the DMSO-only control and the fractional HiBiT signal was plotted versus the log of the PROTAC concentration and fit to a 4-parameter dose-response model to obtain the concentration of the compound that leads to half maximal degradation (DC₅₀) as well as the maximum degradation observed (Dₘₐₓ, conventionally expressed as a percentage of control).

The results of these assays are shown below in Table 1

TABLE 1
HiBiT Degradation Assay Results

| Compound | AsPC-1 HiBiT-KRASG12D | | SW1573 HiBiT-KRASG12C | |
|---|---|---|---|---|
| No. | DC₅₀ (nM)^{a} | D_{Max}^{b} | DC₅₀ (nM)^{c} | D_{Max}^{d} |
| 1 | B | A | D | C |
| 2 | A | A | C | C |
| 3 | D | — | D | — |
| 4 | A | C | D | — |
| 5 | A | A | A | B |
| 6 | C | D | D | — |
| 7 | A | A | A | B |
| 8 | C | C | D | — |
| 9 | A | A | D | — |
| 10 | D | — | D | — |
| 11 | C | B | D | — |
| 12 | C | A | D | — |
| 13 | B | A | D | — |
| 14 | D | — | D | — |
| 15 | C | C | D | — |
| 16 | A | A | D | — |
| 17 | D | — | C | C |
| 18 | D | — | D | — |
| 19 | D | — | C | C |
| 20 | A | A | D | — |
| 21 | A | A | D | — |
| 22 | B | C | C/D* | — |
| 23 | C | C | D | — |
| 24 | A | A | — | — |
| 25 | B | C | — | — |
| 26 | A | A | — | — |
| 27 | A | A | — | — |
| 28 | D | — | — | — |
| 29 | A | A | — | — |
| 30 | A | C | — | — |
| 31 | D | — | — | — |
| 32 | A | A | — | — |
| 33 | A | A | — | — |
| 34 | A | A | D | — |
| 35 | A | A | D | — |
| 36 | A | A | D | — |
| 37 | A | A | D | — |
| 38 | A | A | C | C |
| 39 | A | A | C | C |
| 40 | A | A | D | — |
| 41 | A | C | D | C |
| 42 | A | A | A | A |
| 43 | A | A | D | C |
| 44 | A | D | D | — |
| 45 | A | A | A | B |
| 46 | D | — | D | — |
| 47 | D | — | D | — |
| 48 | A | A | D | — |
| 49 | D | — | — | — |
| 50 | A | A | — | — |
| 51 | D | — | — | — |
| 52 | A | A | — | — |
| 53 | A | A | — | — |
| 54 | A | A | — | — |
| 55 | D | — | — | — |
| 56 | A | A | — | — |
| 57 | A | B | — | — |
| 58 | A | A | — | — |
| 59 | A | A | — | — |
| 60 | A | B | — | — |
| 61 | A | A | — | — |
| 62 | D | — | — | — |
| 63 | A | C | — | — |
| 64 | A | C | — | — |
| 65 | C | D | — | — |
| 66 | A | B | — | — |
| 67 | D | — | — | — |
| 68 | A | A | — | — |
| 69 | A | A | — | — |
| 70 | D | — | — | — |
| 71 | A | A | — | — |
| 72 | D | D | — | — |
| 73 | A | A | — | — |
| 74 | A | A | — | — |
| 75 | C | B | — | — |
| 76 | C | C | — | — |
| 77 | A | A | — | — |
| 78 | C | C | — | — |
| 79 | A | A | — | — |
| 80 | A | A | — | — |
| 81 | A | A | — | — |
| 82 | B | C | — | — |
| 83 | A | A | — | — |
| 84 | C | B | — | — |
| 85 | A | A | — | — |
| 86 | C | A | — | — |
| 87 | B | C | — | — |
| 88 | A | A | — | — |
| 89 | C | D | — | — |
| 90 | A | A | — | — |
| 91 | C | C | — | — |
| 92 | A | A | — | — |
| 93 | C | C | — | — |
| 94 | A | A | — | — |
| 95 | D | — | — | — |
| 96 | A | A | — | — |
| 97 | B | C | — | — |
| 98 | A | A | — | — |
| 99 | D | C | — | — |
| 100 | C | C | — | — |
| 101 | A | B | — | — |
| 102 | A | A | — | — |
| 103 | C | C | — | — |
| 104 | A | A | — | — |
| 105 | A | A | — | — |
| 106 | A | A | — | — |
| 107 | A | A | — | — |
| 108 | A | A | — | — |
| 109 | A | A | — | — |
| 110 | A | A | — | — |
| 111 | A | A | — | — |
| 112 | A | A | — | — |
| 113 | A | C | — | — |
| 114 | A | A | — | — |
| 115 | B | C | — | — |
| 116 | A | A | — | — |
| 117 | A | A | — | — |
| 118 | B | C | — | — |
| 119 | A | C | — | — |
| 120 | A | A | — | — |
| 121 | A | A | — | — |
| 122 | B | C | — | — |
| 123 | A | A | — | — |
| 124 | A | D | — | — |
| 125 | B | C | — | — |
| 126 | A | A | — | — |
| 127 | B | D | — | — |
| 128 | A | A | — | — |
| 129 | A | B | — | — |
| 130 | A | A | — | — |
| 131 | A | D | — | — |
| 132 | A | A | — | — |
| 133 | A | D | — | — |
| 134 | C | C | — | — |
| 135 | A | A | — | — |
| 136 | D | — | — | — |
| 137 | C | C | — | — |
| 138 | A | A | — | — |
| 139 | C | D | — | — |
| 140 | A | A | — | — |
| 141 | C | C | — | — |
| 142 | D | — | — | — |
| 143 | A | A | — | — |
| 144 | B | C | — | — |
| 145 | — | — | — | — |
| 146 | — | — | — | — |
| 147 | — | — | — | — |
| 148 | — | — | — | — |
| 149 | — | — | — | — |
| 150 | — | — | — | — |
| 151 | — | — | — | — |
| 152 | — | — | — | — |
| 153 | A | A | — | — |
| 154 | A | A | — | — |
| 155 | A | B | — | — |
| 156 | A | A | — | — |
| 157 | A | A | — | — |
| 158 | A | A | — | — |
| 159 | A | B | — | — |
| 160 | D | C | — | — |
| 161 | A | A | — | — |
| 162 | A | C | — | — |
| 163 | A | B | — | — |
| 164 | D | C | — | — |
| 165 | A | C | — | — |
| 166 | A | C | — | — |
| 167 | C | C | — | — |
| 168 | A | A | — | — |
| 169 | B | C | — | — |
| 170 | C | C | — | — |
| 171 | C | C | — | — |
| 172 | A | A | — | — |
| 173 | A | A | — | — |
| 174 | A | A | — | — |
| 175 | A | A | — | — |
| 176 | A | A | — | — |
| 177 | A | B | — | — |
| 178 | A | A | — | — |
| 179 | A | A | — | — |
| 180 | D | — | — | — |
| 181 | D | — | — | — |
| 182 | A | C | — | — |
| 183 | A | A | — | — |
| 184 | A | A | — | — |
| 185 | A | A | — | — |
| 186 | A | A | — | — |
| 187 | A | A | — | — |
| 188 | C | C | — | — |
| 189 | D | — | — | — |
| 190 | A | A | — | — |
| 191 | A | A | — | — |
| 192 | D | — | — | — |
| 193 | D | — | — | — |
| 194 | A | A | — | — |
| 195 | C | D | — | — |
| 196 | B | C | — | — |
| 197 | C | D | — | — |
| 198 | A | A | — | — |
| 199 | A | A | — | — |
| 200 | A | B | — | — |
| 201 | D | — | — | — |
| 202 | A | A | — | — |
| 203 | C | C | — | — |
| 204 | A | A | — | — |
| 205 | C | C | — | — |
| 206 | A | A | — | — |
| 207 | D | — | — | — |
| 208 | A | A | — | — |
| 209 | A | A | — | — |
| 210 | B | C | — | — |
| 211 | A | A | — | — |
| 212 | A | A | — | — |
| 213 | A | A | — | — |
| 214 | A | A | — | — |
| 215 | A | A | — | — |
| 216 | C | C | — | — |
| 217 | C | C | — | — |
| 218 | A | A | — | — |
| 219 | C | C | — | — |
| 220 | A | A | — | — |
| 221 | D | — | — | — |
| 222 | A | B | — | — |
| 223 | A | A | — | — |
| 224 | B | C | — | — |
| 225 | A | A | — | — |
| 226 | A | A | — | — |
| 227 | A | A | — | — |
| 228 | A | A | — | — |
| 229 | A | A | — | — |
| 230 | A | B | — | — |
| 231 | A | A | — | — |
| 232 | C | C | — | — |
| 233 | A | A | — | — |
| 234 | C | C | — | — |
| 235 | A | A | — | — |
| 236 | D | — | — | — |
| 237 | D | — | — | — |
| 238 | A | A | — | — |
| 239 | A | A | — | — |
| 240 | D | — | — | — |
| 241 | A | B | — | — |
| 242 | D | — | — | — |
| 243 | A | A | — | — |
| 244 | A | A | — | — |
| 245 | D | — | — | — |
| 246 | D | — | — | — |
| 247 | D | — | — | — |
| 248 | D | — | — | — |
| 249 | A | A | — | — |
| 250 | A | A | — | — |
| 251 | A | A | — | — |
| 252 | A | A | — | — |
| 253 | C | C | — | — |
| 254 | B | C | — | — |
| 255 | B | C | — | — |
| 256 | A | A | — | — |
| 257 | A | A | — | — |
| 258 | A | A | — | — |
| 259 | A | A | — | — |
| 260 | A | A | — | — |
| 261 | A | A | — | — |
| 262 | A | A | — | — |
| 263 | A | A | — | — |
| 264 | A | A | — | — |
| 265 | C | B | — | — |
| 266 | A | A | — | — |
| 267 | A | A | — | — |
| 268 | A | A | — | — |
| 269 | A | B | — | — |
| 270 | A | A | — | — |
| 27 | A | C | — | — |
| 272 | A | B | — | — |
| 273 | A | A | — | — |
| 274 | A | B | — | — |
| 275 | A | A | — | — |
| 276 | A | A | — | — |
| 277 | C | C | — | — |
| 278 | A | A | — | — |
| 279 | A | B | — | — |
| 280 | B | C | — | — |
| 281 | A | A | — | — |
| 282 | B | D | — | — |
| 283 | A | A | — | — |
| 284 | D | — | — | — |
| 285 | A | A | — | — |
| 286 | A | A | — | — |
| 287 | C | D | — | — |
| 288 | A | A | — | — |
| 289 | C | C | — | — |
| 290 | D | — | — | — |
| 291 | A | D | — | — |
| 292 | A | A | — | — |
| 293 | A | B | — | — |
| 294 | A | A | — | — |
| 295 | A | A | — | — |
| 296 | A | A | — | — |
| 297 | A | D | — | — |
| 298 | D | — | — | — |
| 299 | A | A | — | — |
| 300 | A | A | — | — |
| 301 | A | A | — | — |
| 302 | C | C | — | — |
| 303 | A | B | — | — |
| 304 | D | — | — | — |
| 305 | B | C | — | — |
| 306 | A | A | — | — |
| 307 | A | A | — | — |
| 308 | A | B | — | — |
| 309 | A | D | — | — |
| 310 | A | A | — | — |
| 311 | A | A | — | — |
| 312 | C | C | — | — |
| 313 | A | A | — | — |
| 314 | A | A | — | — |
| 315 | D | — | — | — |
| 316 | A | C | — | — |
| 317 | A | A | — | — |
| 318 | C | C | — | — |
| 319 | C | B | — | — |
| 320 | A | A | — | — |
| 321 | B | C | — | — |
| 322 | B | B | — | — |
| 323 | A | B | — | — |
| 324 | A | A | — | — |
| 325 | A | A | — | — |
| 326 | A | D | — | — |
| 327 | A | A | — | — |
| 328 | A | A | — | — |
| 329 | A | A | — | — |
| 330 | A | B | — | — |
| 331 | D | — | — | — |
| 332 | B | A | — | — |
| 333 | A | A | — | — |
| 334 | A | B | — | — |
| 335 | A | A | — | — |
| 336 | C | C | — | — |
| 337 | A | A | — | — |
| 338 | A | A | — | — |
| 339 | D | — | — | — |
| 340 | A | A | — | — |
| 341 | D | — | — | — |
| 342 | A | A | — | — |
| 343 | A | A | — | — |

*= data were truncated due to anomalous effects at concentrations between C and D.
— = not calculated
^{a}A ≤ 600 nM; 600 nM < B ≤ 1000 nM; 1000 nM < C ≤ 3000 nM; D > 3000 nM
^{b}80% < A ≤ 100%; 60% < B ≤ 80%; 20% <C ≤ 60%; D ≤ 20%
^{c}A ≤ 600 nM; 600 nM < B ≤ 1000 nM; 1000 nM < C ≤ 3000 nM; D > 3000 nM
^{d}80% < A ≤ 100%; 60% < B ≤ 80%; 20% < C ≤ 60%; D ≤ 20%

EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

The methods of the disclosure have been described herein by reference to certain preferred embodiments. However, as particular variations thereon will become apparent to those skilled in the art, based on the disclosure set forth herein, the disclosure is not to be considered as limited thereto.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise.

It is to be understood that at least some of the descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

Further, to the extent that a method does not rely on the particular order of steps set forth herein, the particular order of the steps recited in a claim should not be construed as a limitation on that claim.

All patents, patent applications, references and publications cited herein are fully and completely incorporated by reference as if set forth in their entirety. Such documents are not admitted to be prior art to the present disclosure.

1. A bifunctional compound having the structure of Formula (I): 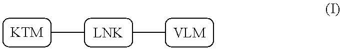
or a pharmaceutically acceptable salt thereof,
wherein:
(a) KTM has the structure of formula KTM-I: 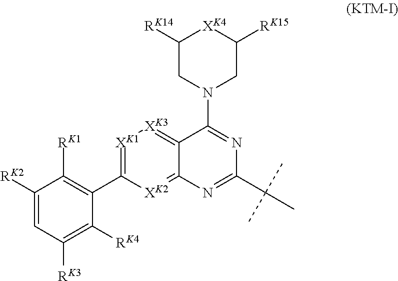
wherein:
X^{K1} is N or CR^{K5};
X^{K2} is N or CR^{K6};
X^{K3} is N or CR^{K7};
X^{K4} is NR^{K8} or C₁-C₃ alkylene, wherein the alkylene is optionally substituted with one or more R^{K9}
R^{K1} and R^{K2} are each independently selected from H, OH, Cl, F, Br, I, C₁-C₆ alkyl, C₁-C₆ haloalkyl, O—(C₁-C₆ alkyl), and O—(C₁-C₆ haloalkyl);
R^{K3} and R^{K4} are each independently selected from H, OH, Cl, F, Br, I, C₁-C₆ alkyl, C₁-C₆ haloalkyl, C₃-C₁₀ cycloalkyl, 3- to 10-membered heterocycle, O—(C₁-C₆ alkyl), and O—(C₁-C₆ haloalkyl); or, alternatively,
R^{K3} and R^{K4}, together with the carbons to which they are bonded, form C₆-C₁₀ aryl or 5- to 6-membered heteroaryl, wherein the aryl or heteroaryl is optionally substituted with one, two, three, four, or five R^{K11};
R^{K5}, R^{K6}, and R^{K7} are each independently selected from H, Cl, F, Br, I, NR^{K12}R^{K13}, C₁-C₆ alkyl, and C₁-C₆ haloalkyl;
R^{K8} and R^{K9} are each independently selected from H, C₁-C₆ alkyl, and C₁-C₆ haloalkyl;
each R^{K11} is independently selected from H, OH, CN, CI, F, Br, I, NR^{K12}R^{K13}, C₁-C₆ alkyl, C₂-C₆ alkenyl, C₂-C₆ alkynyl, and C₁-C₆ haloalkyl;
R^{K12} and R^{K13} are each independently selected from H, C₁-C₆ alkyl, and C₁-C₆ haloalkyl;
R^{K14} and R^{K15} are each independently selected from H, C₁-C₆ alkyl, and C₁-C₆ haloalkyl; or alternatively,
R^{K14} and R^{K15}, together with X^{K4} and the carbons to which they are bonded, form a C₄-C₇cycloalkyl or 4- to 7-membered heterocycle; and [symbol]
represents the attachment point between KTM and LNK;
(b) LNK is a chemical linking moiety that covalently couples the KTM to the VLM, having the structure L-I: 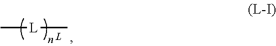
wherein:
each L is independently selected from 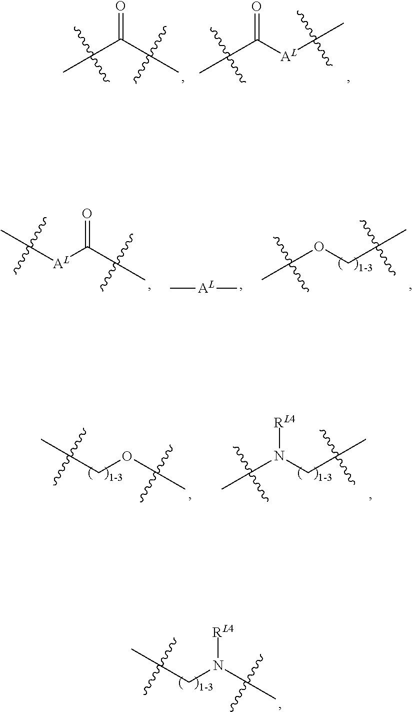
C₂-C₆ alkylene, C₂-C₆ alkenylene, C₂-C₆ alkynylene, monocyclic C₄-C₁₀ cycloalkylene, fused bicyclic C₄-C₁₂ cycloalkylene, bridged bicyclic C₆-C₁₀ cycloalkylene, or spiro-fused bicyclic C₅-C₁₂ cycloalkylene, monocyclic 4-10 membered heterocycloalkylene, fused bicyclic 4-10 membered heterocycloalkylene, bridged bicyclic 6-10 membered heterocycloalkylene, spiro-fused 5-12 membered heterocycloalkylene, C₆-C₁₀ arylene, and 5-6 membered heteroarylene, wherein each cycloalkylene, heterocycloalkylene, arylene, and heteroarylene is optionally substituted with one, two, three, four, or five R^{L5};
wherein each A^{L} is independently selected from CR^{L1}R^{L2}, NR^{L3}, and O;
each R^{L1} and R^{L2} is independently selected from H, C₁-C₆ alkyl, O—(C₁-C₆ alkyl), and C₁-C₆ haloalkyl, wherein the alkyl is optionally substituted with Cl, F, OH, NH₂, CN, or CF₃;
each R^{L3} is independently selected from H, C₁-C₆ alkyl, O—(C₁-C₆ alkyl), and C₁-C₆ haloalkyl wherein the alkyl is optionally substituted with Cl, F, OH, NH₂, CN, or CF₃;
each R^{L4} is independently selected from C₁-C₆ alkyl, O—(C₁-C₆ alkyl), C₁-C₆ haloalkyl, NH, CN, CF₃, Cl, F, Br, I, and OH wherein the alkyl is optionally substituted with Cl, F, OH, NH₂, CN, or CF₃;
each R^{L5} is independently selected from Cl, F, Br, I, C₁-C₆ alkyl, O—(C₁-C₆ alkyl), C₁-C₆ haloalkyl, NH₂, CN, CF₃, and OH wherein the alkyl is optionally substituted with Cl, F, OH, NH₂, CN, or CF₃;
n^{L} is 2, 3, 4, 5, or 6;
(c) VLM has the structure VLM-I: 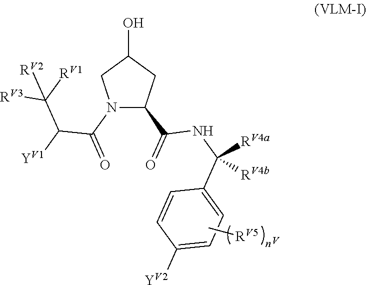
wherein:
Y^{V1} is 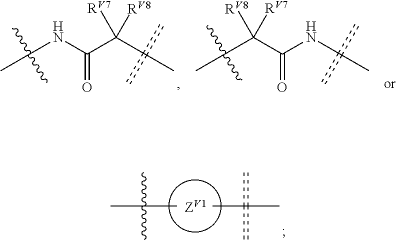
Y^{V2} is CN or 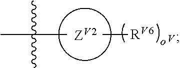 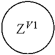
is phenyl or 5- to 6-membered heteroarylene; 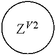
is 5-membered heteroaryl with one or two heteroatoms independently selected from N, S, and O;
R^{V1}, R^{V2}, and R^{V3} are each independently selected from H, C₁-C₆ alkyl, and C₁-C₆ haloalkyl; or, alternatively
R^{V1} and R^{V2}, together with the carbon to which they are bonded, form C₃-C₁₀ cycloalkyl or 5- to 6-membered heterocycle; and R^{V3} is selected from H, C₁-C₆ alkyl, and C₁-C₆ haloalkyl;
R^{V4a} and R^{V4b} are each independently selected from H, C₁-C₆ alkyl, and C₁-C₆ haloalkyl;
each R^{V5} and R^{V6} is independently selected from H and C₁-C₆ alkyl;
R^{V7} and R^{V8} are each independently selected from H, C₁-C₆ alkyl, and C₁-C₆ haloalkyl; or, alternatively,
R^{V7} and R^{V8}, together with the atom to the carbon to which they are bonded, form C₃-C₁₀ cycloalkyl or 5- to 6-membered heterocycle;
n^{V} is 0, 1, 2, 3, or 4;
o^{V} is 0, 1, 2, or 3; and
wherein [symbol] represents the attachment point between VLM and LNK.

2. The bifunctional compound of claim 1, wherein the KTM has the structure of formula (KTM-Ia), (KTM-Ib), (KTM-Ic), (KTM-Id), or (KTM-Ie),

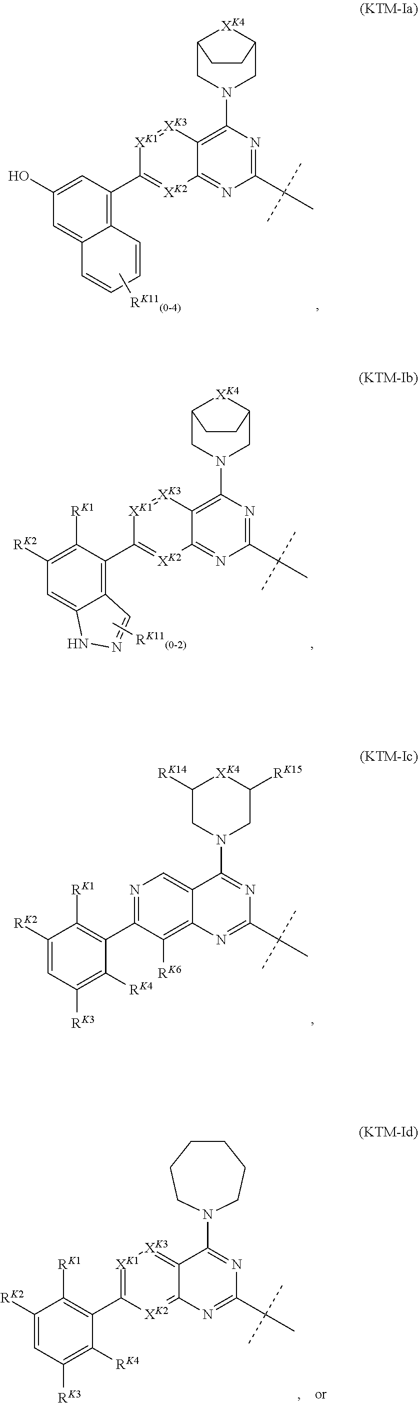

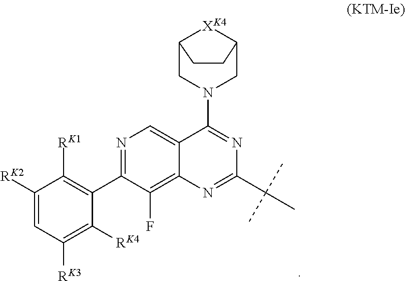

3. The bifunctional compound of claim 1, wherein X^{K2} is CR^{K6} and R^{K6} is F.

4. The bifunctional compound of claim 1, wherein X^{K4} is NH.

5. The bifunctional compound of claim 1, wherein X^{K4} is CH₂.

6. The bifunctional compound of claim 1, wherein X^{K4} is CH₂CH₂.

7. The bifunctional compound of claim 1, wherein one of R^{K3} and R^{K4} is selected from CF₃ and O—CF₃ and the other of R^{K3} and R^{K4} is H.

8. The bifunctional compound of claim 1, wherein KTM has a structure selected from (KTM-1), (KTM-2), (KTM-3), (KTM-4), (KTM-5), (KTM-6), and (KTM-7):

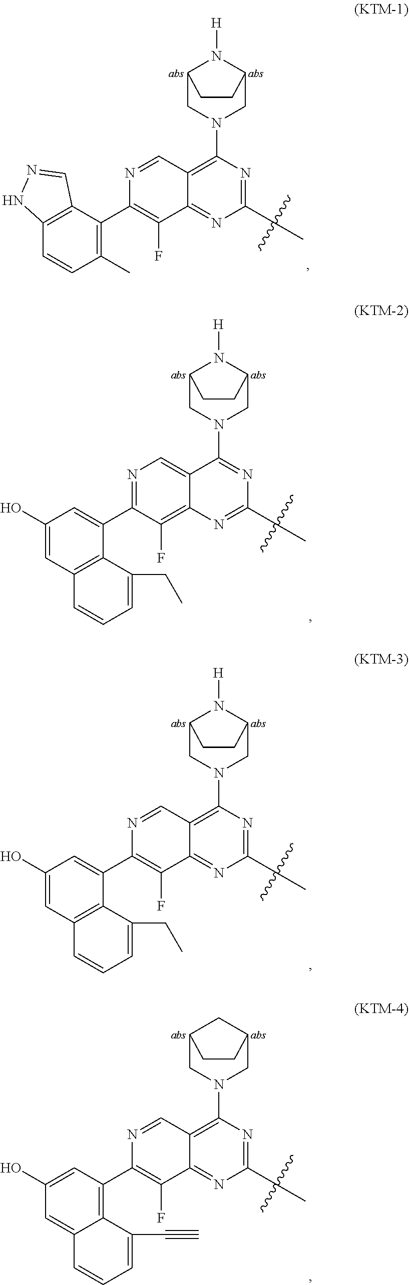 [SMILES: *c1nc(N2CC3CCC(C2)N3)c2cnc(-c3c(C)ccc4[nH]ncc34)c(F)c2n1 ; *c1nc(N2CC3CCC(C2)N3)c2cnc(-c3cc(O)cc4cccc(CC)c34)c(F)c2n1 ; *c1nc(N2CC3CCC(C2)N3)c2cnc(-c3cc(O)cc4cccc(CC)c34)c(F)c2n1 ; *c1nc(N2CC3CCC(C3)C2)c2cnc(-c3cc(O)cc4cccc(C#C)c34)c(F)c2n1]

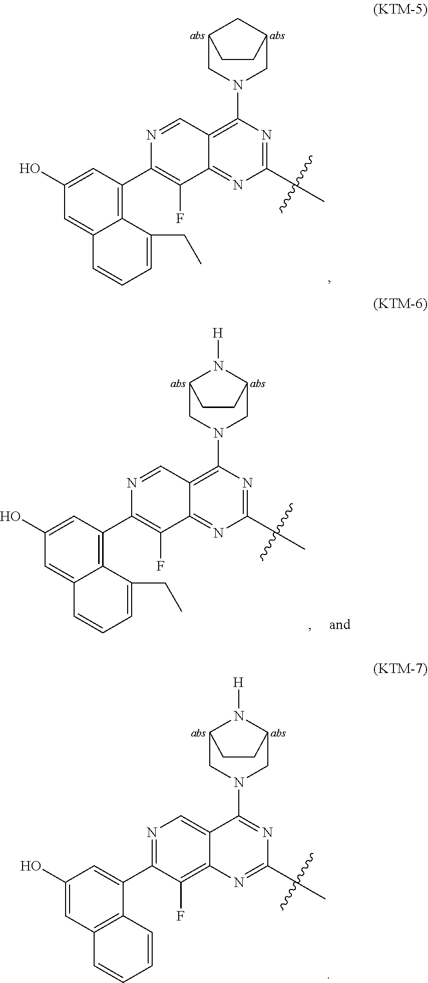 [SMILES: *c1nc(N2CC3CCC(C3)C2)c2cnc(-c3cc(O)cc4cccc(CC)c34)c(F)c2n1 ; *c1nc(N2CC3CCC(C2)N3)c2cnc(-c3cc(O)cc4cccc(CC)c34)c(F)c2n1 ; *c1nc(N2CC3CCC(C2)N3)c2cnc(-c3cc(O)cc4ccccc34)c(F)c2n1]

9. The bifunctional compound of claim 1, wherein LNK has the structure (L-Ia), (L-Ib), (L-Ic), (L-Id), (L-Ie), or (L-If):

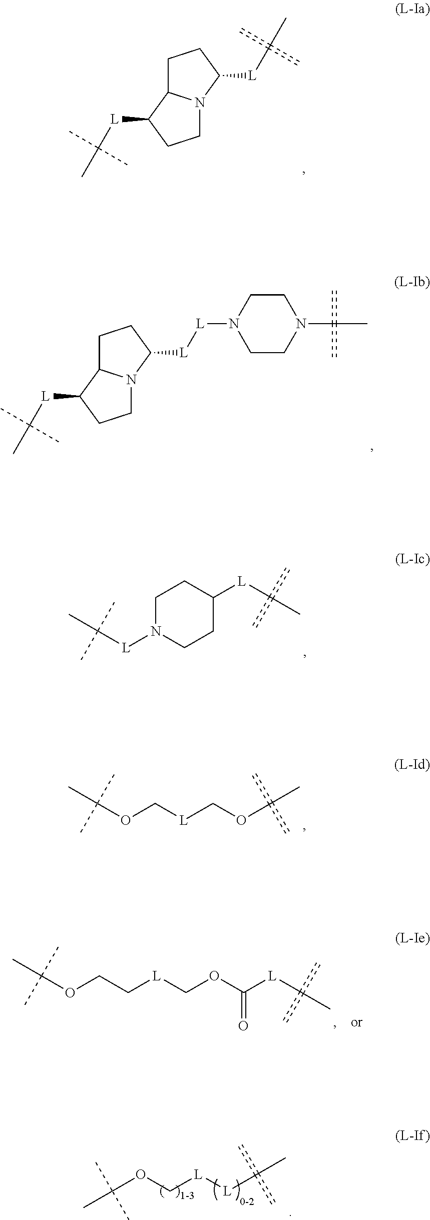

10. The bifunctional compound of claim 9, wherein the LNK has the structure (L-Ia), (L-Ib), or (L-Ic).

11. The bifunctional compound of claim 1, wherein LNK has a structure selected from (LNK-1), (LNK-2), (LNK-3), (LNK-4), (LNK-5), (LNK-6), (LNK-7), (LNK-8), (LNK-9), and (LNK-10):

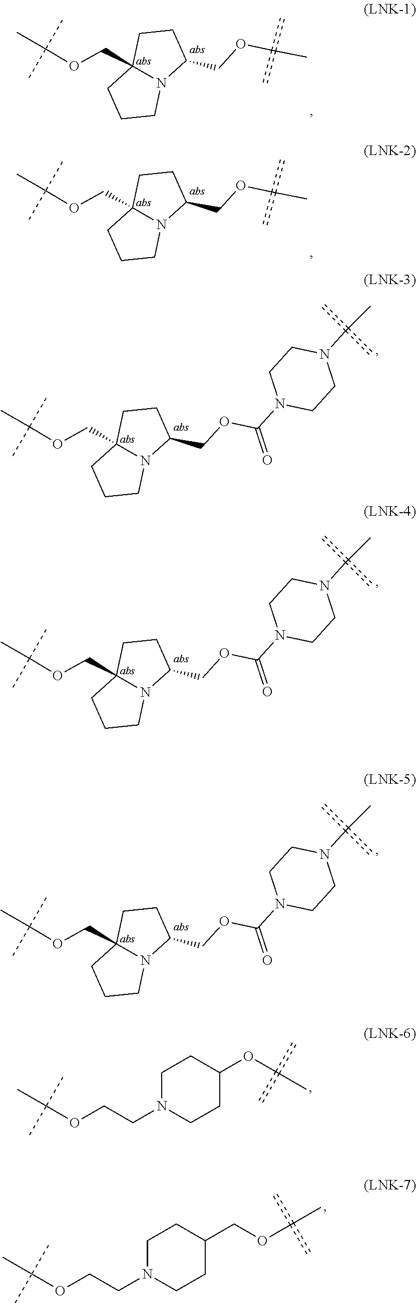

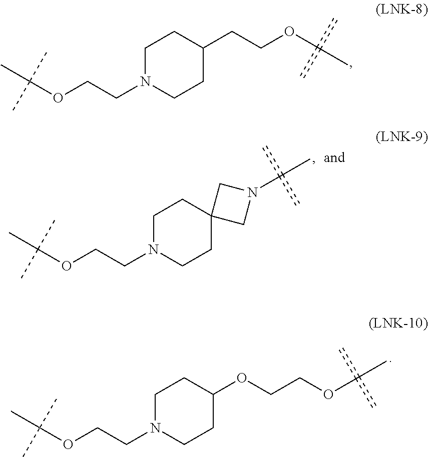

12. The bifunctional compound of claim 1, wherein VLM has the structure (VLM-Ia), (VLM-Ib), (VLM-Ic), or (VLM-Id):

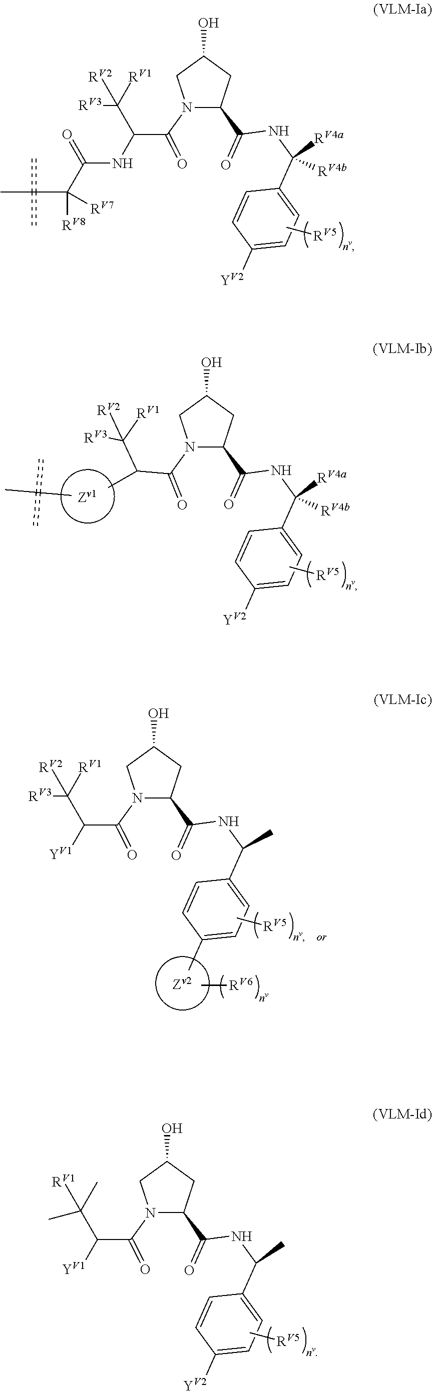

13. The bifunctional compound of claim 1, wherein Y^{V2} is

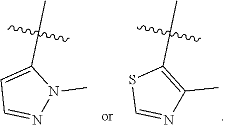

14. The bifunctional compound of claim 1, wherein VLM has a structure selected from (VLM-1), (VLM-2), (VLM-3), (VLM-4), (VLM-5), (VLM-6), (VLM-7), (VLM-8), (VLM-9), and (VLM-10):

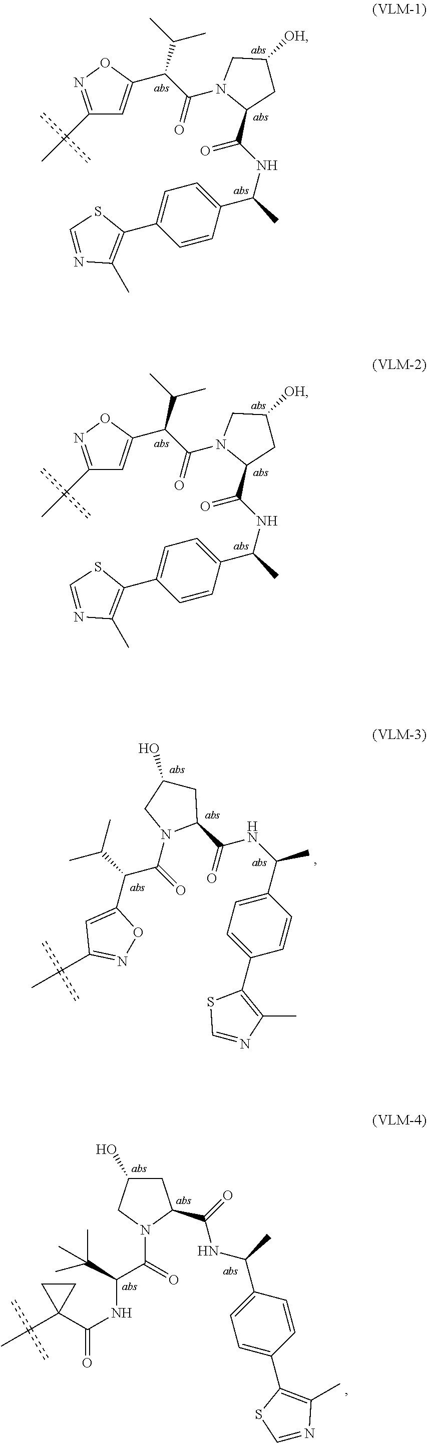

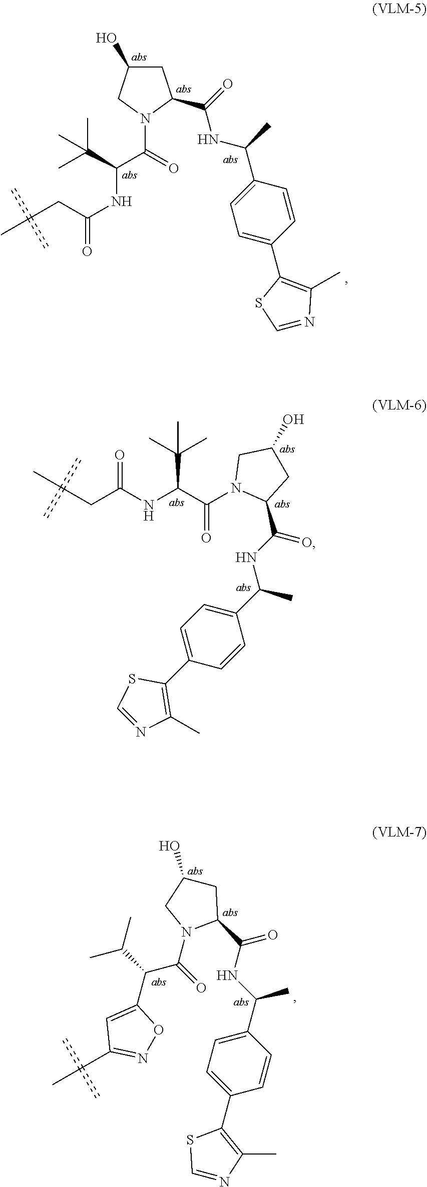

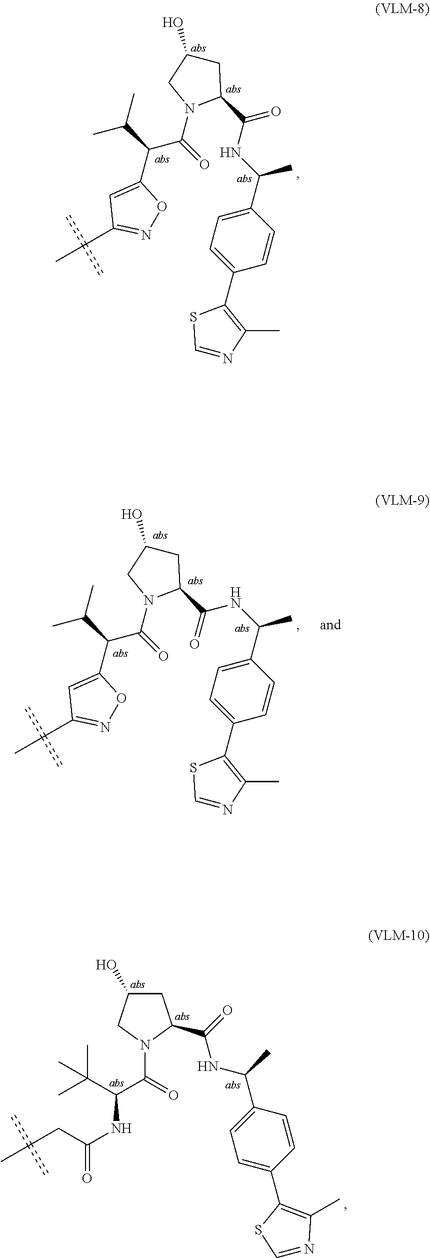

15. The bifunctional compound of claim 1, wherein the compound is selected from Compounds 1-71, or a pharmaceutically acceptable salt thereof.

16. The bifunctional compound of claim 1, wherein the KTM binds to KRAS reversibly.

17. The bifunctional compound of claim 1 wherein KRAS contains a mutation relative to wild type, wherein the mutation is G12C, G12D or G12V.

18. (canceled)

19. (canceled)

20. A pharmaceutical composition comprising the bifunctional compound of claim 1 and one or more pharmaceutically acceptable excipients.

21. A method of treating a disease or disorder in a subject, the method comprising administering to the subject in need thereof a therapeutically effective amount of a bifunctional compound of claim 1, wherein the disease or disorder is pancreatic cancer, colon cancer, colorectal cancer, lung cancer, non-small cell lung cancer, endometrial cancer, cervical cancer, bladder cancer, liver cancer, myeloid leukemia, ovarian cancer or breast cancer.

22. (canceled)

23. (canceled)

24. (canceled)

25. A method of treating a disease or disorder in a subject, the method comprising administering to the subject in need thereof a therapeutically effective amount of the pharmaceutical composition of claim 20, wherein the disease or disorder is pancreatic cancer, colon cancer, colorectal cancer, lung cancer, non-small cell lung cancer, endometrial cancer, cervical cancer, bladder cancer, liver cancer, myeloid leukemia, ovarian cancer or breast cancer.
